(19) United States
(12) Patent Application Publication
(10) Pub. No.: US 2006/0014165 A1
(43) Pub. Date: Jan. 19, 2006

(54) Methods of diagnosis and treatment for asthma and other respiratory diseases based on haplotype association

(75) Inventors: Hakon Hakonarson, Reykjavik (IS); Mark E. Gurney, East Grand Rapids, MI (US); Eva Halapi, Reykjavik (IS)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: deCODE genetics ehf., Reykjavik (IS)

(21) Appl. No.: 11/043,752

(22) Filed: Jan. 26, 2005

Related U.S. Application Data
(63) Continuation-in-part of application No. PCT/US04/22446, filed on Jul. 14, 2004.
(60) Provisional application No. 60/487,072, filed on Jul. 14, 2003.
(60) Provisional application No. 60/559,611, filed on Apr. 5, 2004.

(51) Int. Cl. C12Q 1/68; A61K 31/47

(52) U.S. Cl. 435/6; 514/313

(57) ABSTRACT

Methods for diagnosis of asthma or a susceptibility to asthma based on detection of at-risk haplotypes associated with MAP3K9 are disclosed. Also methods for treatment of asthma or a susceptibility to asthma based on detection of at-risk haplotypes associated with MAP3K9 are disclosed. In particular, pathway targeting for treating individuals who are at-risk of developing asmtha are described. In certain aspects, MLK1 inhibitors are used in treatment methods.

### RELATED APPLICATIONS

This application is a continuation-in-part of International Application No. PCT/US2004/022446, which designated the United States and was filed on Jul. 14, 2004 and claims the benefit of U.S. Provisional Application No. 60/487,072, filed on Jul. 14, 2003 and U.S. Provisional Application No. 60/559,611, filed on Apr. 5, 2004. The entire teachings of the above applications are incorporated herein by reference.

### BACKGROUND OF INVENTION

Bronchial asthma [Morbidity Number (MIM) 600807], the most common chronic disease affecting children and young adults, is a complex genetic disorder with several overlapping phenotypes (Cookson and Moffatt 2000; Weiss 2001). There is strong evidence for a genetic component in asthma (Bleecker et al., 1997; Kauffmann et al., 2002). Multiple environmental factors are also known to modulate the clinical expression of asthma as well as the asthma-associated phenotypes: bronchial hyperresponsiveness, atopy and elevated IgE (Koppelman et al., 1999; Cookson 1999; Holloway et al., 1999). It is a commonly held view that asthma is caused by multiple interacting genes some having a protective effect and others contributing to the disease pathogenesis, with each gene having its own tendency to be influenced by the environment (Koppelman et al. and Postma, 1999; Cookson, 1999; Holloway et al., 1999). Thus, the complex nature of the asthma phenotype, together with substantial locus heterogeneity and environmental influence, has made it difficult to uncover genetic factors that underlie asthma.

Numerous loci and candidate genes have been reported showing linkage and association to asthma and atopy. While some studies reporting these observations are compelling, no asthma gene conferring high risk has been mapped such that it meets stringent criteria for genome-wide significance.

### SUMMARY OF THE INVENTION

As described herein, a gene on chromosome 14q24 has been identified that plays a major role i][asthma. The gene MAP3K9 (the asthma gene) encodes a kinase that is part of the Mixed Lineage Kinase (MLK) family. The protein encoded by the MAP3K9 gene is designated as MAP3K9 (herein) or more commonly as MLK-1. This locus has been designated asthma locus one (hereinafter referred to as “AS 1”). The present invention relates to methods of treatment using inhibitors to the asthma gene products.

The invention pertains to methods of treatment (prophylactic and/or therapeutic) for certain diseases and conditions (e.g., asthma and other respiratory diseases) associated with MAP3K9 or with other members of the JNK pathway, for example, members of the MLK family kinases (e.g., MLK1, MLK2, MLK3(SPRK, PTK1), MLK4, LZK, DLK (ZPK, MUK) and MLK6), in particular, MLK1; and/or with other members of the JNK pathway (as shown in FIG. 1), receptors and/or binding agents of the enzymes; the transcription factor AP-1 and its individual components, c-jun and v-fos, and receptors for the MLK family kinases. The methods include the following: methods of treatment for asthma or susceptibility to asthma; and methods of treatment for respiratory diseases associated with MAP3K9 or with other members of the MLK family.

In the methods of the invention, a MLK kinase family inhibitor is administered to an individual in a therapeutically effective amount. The MLK kinase family inhibitor can be an agent that inhibits or antagonizes a member of the JKN pathway, in particular the MLK family kinase pathway (e.g., MLK1, MLK2, MLK3) that are members of a subset of the JNK pathway, and the transcription factor AP-1 and its individual components, c-jun and v-fos. For example, the MLK kinase family inhibitor synthesis inhibitor can be an agent that inhibits or antagonizes MAP3K9 polypeptide (MLK1) activity (e.g., a MAP3K9 inhibitor, for example, a compound (1), CEP-1347, or a compound of Formula IV) and/or MAP3K9 nucleic acid expression, as described herein (e.g., a MAP3K9 nucleic acid antagonist). In one aspect, the agent alters activity and/or nucleic acid expression of MAP3K9. In another aspect, the agents used in the methods are represented by formula I and further described in Tables A and B, and their optically pure stereoisomers, mixtures of stereoisomers, salts, chemical derivatives, and analogues. In other aspects, the agent used in the methods is CEP-1347 as shown in Formula III, its optically pure stereoisomers, mixtures of stereoisomers, salts, chemical derivatives, and analogues or a compound of Structural Formula IV, its optically pure stereoisomers, mixtures of stereoisomers, salts, chemical derivatives, and analogues. In another aspect, the agent alters metabolism or inhibits activity of an MLK1 protein (e.g., MLK1 kinase), or an MLK kinase family member.

In certain aspects of the invention, the individual is an individual who has at least one risk factor, such as an at-risk haplotype for asthma; an at-risk haplotype in the MAP3K9 gene; a polymorphism in a MAP3K9 nucleic acid; dysregulation of MAP3K9 mRNA expression; dysregulation of a MAP3K9 mRNA isoform; increased MLK1 protein expression; increased MLK1 biochemical activity; and increased MKL1 protein isoform expression.

The invention further pertains to methods of assessing response to treatment with a MLK kinase family protein, for example MLK1, by assessing a level of a MLK kinase family protein in the individual before treatment, and comparing the level to a level of the MLK kinase family protein assessed during or after treatment. A level that is significantly lower during or after treatment, than before treatment, is indicative of efficacy of the treatment with the MLK kinase family protein. The level of the MLK kinase family protein can be measured using a biochemical assay of enzyme activity, or using methods that allow direct quantitation of the amount of MLK kinase protein, e.g., by enzyme-linked immunosorbent assay (EIA). The invention additionally pertains to methods of assessing response to treatment with a MLK kinase family protein, by stimulating production of a MLK kinase family protein or a MLK kinase family protein in a first test sample from the individual (e.g., a sample comprising leukocytes) before treatment, and comparing the level of the MLK kinase family protein with a level of production of the MLK kinase family protein in a second test sample from the individual, during or after treatment. A level of production of the MLK kinase family protein or MLK kinase family protein in the second test sample that is significantly lower than the level in the first test sample is indicative of efficacy of the treatment. Similarly, the invention encompasses methods of assessing response to treatment with a MLK kinase family inhibitor, by assessing a level of an inflammatory marker (e.g., IL-2 and TNFα) in the individual before treatment, and during or after treatment. A level of the inflammatory protein marker during or after treatment, that is significantly lower than the level of inflammatory marker before treatment, is indicative of efficacy of the treatment. The first sample can also be a “control level” of the MLK kinase family protein that has been determined by large sampling of individual without any incidence of asthma.

The present invention also relates to isolated nucleic acid molecules comprising the asthma gene located within AS1 locus. It has also been discovered that particular combinations of genetic markers (“haplotypes”), are present at a higher than expected frequency in patients with phenotypes associated with asthma and a susceptibility to asthma. The markers that are included in the haplotypes described herein are associated with the genomic region that directs expression of MAP3K9 kinase.

In one embodiment, the invention is directed to a method of diagnosing asthma or a susceptibility to asthma in an individual, comprising detecting the presence or absence of an at-risk haplotype, comprising a haplotype selected from the group consisting of: haplotype 1, haplotype 2, haplotype 3, haplotype 4, haplotype 5, haplotype 6, haplotype 7, haplotype 8, haplotype 9, and haplotype 10 and combinations thereof, wherein the presence of the haplotype is indicative of asthma or a susceptibility to asthma. In a particular embodiment, the invention is directed to assaying for the presence of a first nucleic acid molecule in a sample, comprising contacting said sample with a second nucleic acid molecule comprising the one or more haplotypes described herein. In one embodiment, determining the presence or absence of the haplotype comprises enzymatic amplification of nucleic acid from the individual. In a particular embodiment, determining the presence or absence of the haplotype further comprises electrophoretic analysis. For example, in one embodiment, determining the presence or absence of the haplotype comprises restriction fragment length polymorphism analysis. In another embodiment, determining the presence or absence of the haplotype comprises sequence analysis.

In a particular embodiment, determining the presence or absence of the haplotype further comprises electrophoretic analysis. For example, in one embodiment, determining the presence or absence of the haplotype comprises restriction fragment length polymorphism analysis. In another embodiment, determining the presence or absence of the haplotype comprises sequence analysis.

In another embodiment, the invention is directed to a method of diagnosing asthma or a susceptibility to asthma in an individual, comprising detecting the presence or absence of an at-risk haplotype comprising haplotype 10 (shown in Table 1), wherein the presence of the haplotype is indicative of asthma or a susceptibility to asthma. In a particular embodiment, determining the presence or absence of the haplotype comprises enzymatic amplification of nucleic acid from the individual. In a particular embodiment, determining the presence or absence of the haplotype further comprises electrophoretic analysis. For example, in one embodiment, determining the presence or absence of the haplotype comprises restriction fragment length polymorphism analysis. In another embodiment, determining the presence or absence of the haplotype comprises sequence analysis.

In another embodiment, the invention is directed to a kit for assaying a sample for the presence of a haplotype associated with asthma, wherein the haplotype comprises two or more specific alleles, and wherein the kit comprises one or more nucleic acids capable of detecting the presence or absence of two or more of the specific alleles, thereby indicating the presence or absence of the haplotype in the sample. In a particular embodiment, the nucleic acid comprises a contiguous nucleotide sequence that is completely complementary to a region comprising specific allele of the haplotype.

In another embodiment, the invention is directed to a reagent kit for assaying a sample for the presence of a haplotype associated with asthma, wherein the haplotype comprises two or more specific alleles, comprising in separate containers: a) one or more labeled nucleic acids capable of detecting one or more specific alleles of the haplotype; and b) reagents for detection of said label. In a particular embodiment, the labeled nucleic acid comprises a contiguous nucleotide sequence that is completely complementary to a region comprising specific allele of the haplotype.

In yet another embodiment, the invention is directed to a reagent kit for assaying a sample for the presence of a haplotype associated with asthma, wherein the haplotype comprises two or more specific alleles, wherein the kit comprises one or more nucleic acids comprising a nucleotide sequence that is at least partially complementary to a part of the nucleotide sequence of the MAP3K9 gene, and wherein the nucleic acid is capable of acting as a primer for a primer extension reaction capable of detecting one or more of the specific alleles of the haplotype.

In another embodiment, the invention is directed to a method for the diagnosis and identification of susceptibility to asthma in an individual, comprising: screening in a sample from the individual to be diagnosed for an at-risk haplotype associated with MAP3K9 that is more frequently present in an individual susceptible to asthma compared to an individual who is not susceptible to asthma wherein the at-risk haplotype increases the risk significantly. In a particular embodiment, the significant increase is at least about 20%. In another embodiment, the significant increase is identified as an odds ratio of at least about 1.2.

The invention further pertains to a method of diagnosing asthma or a susceptibility to asthma in an individual, comprising detecting in a sample from the individual to be diagnosed the presence or absence of at least one marker of an at-risk haplotype associated with the MAP3K9 gene selected from the group consisting of: DG14S205, DG14S428, D14S1002, DG14S4399, DG14S404, D14S251, DG14S1300, DG14S266, DG14DG14S448, DG14S1879, DG14S417, SG14S89, SG14S152, SG14S174 and SG14S184, wherein the presence of one or more markers is indicative of asthma or a susceptibility to asthma.

In another embodiment, the invention is directed to a method for diagnosing a susceptibility to asthma in an individual, comprising determining in a sample from the individual to be diagnosed the presence or absence in the individual of a haplotype, comprising two or more alleles selected from the group consisting of one or a combination of the markers that comprise the haplotypes set forth in Table 1, wherein the presence of the haplotype is indicative of susceptibility to asthma. In a particular embodiment, determining the presence or absence of the haplotype further comprises electrophoretic analysis. For example, in one embodiment, determining the presence or absence of the haplotype comprises restriction fragment length polymorphism analysis. In another embodiment, determining the presence or absence of the haplotype comprises sequence analysis.

In yet another embodiment, the invention is directed to a method for diagnosing a susceptibility to asthma in an individual, comprising obtaining a nucleic acid sample from the individual; and analyzing the nucleic acid sample for the presence or absence of a haplotype comprising two or more alleles selected from the group consisting of one or a combination of the markers that comprise the haplotypes set forth in Table 1, wherein the presence of the haplotype is indicative of susceptibility to asthma.

The present invention relates to isolated nucleic acid molecules comprising the asthma gene located within AS1 locus. In one embodiment, the isolated nucleic acid molecule comprises a nucleotide sequence of SEQ ID NO: 1 or the complement thereof; wherein the nucleic acid molecule can optionally comprise one ore more of the SNPs set forth in the Examples. The invention further relates to a nucleic acid molecule that hybridizes under high stringency conditions to a nucleotide sequence of SEQ ID NO: 1 and the complement thereof. The invention additionally relates to isolated nucleic acid molecules (e.g., cDNA molecules) encoding a MAP3K9 polypeptide (e.g., encoding a polypeptide of SEQ ID NO: 2).

Also contemplated by the invention is a method of assaying for the presence of a first nucleic acid molecule in a sample, comprising contacting said sample with a second nucleic acid molecule, where the second nucleic acid molecule comprises at least one (or more) nucleic acid sequence(s) selected from the sequences described herein, wherein the nucleic acid sequence hybridizes to the first nucleic acid under high stringency conditions. In certain embodiments, the second nucleic acid molecule contains one or more polymorphism(s), described herein.

The invention also relates to a vector comprising an isolated nucleic acid molecule of the invention, optionally including one or more of the polymorphisms described herein, operably linked to a regulatory sequence, as well as to a recombinant host cell comprising the vector. The invention also provides a method for producing a polypeptide encoded by an isolated nucleic acid molecule having a polymorphism, comprising culturing the recombinant host cell under conditions suitable for expression of the nucleic acid molecule.

Also contemplated by the invention is a method of assaying for the presence of a polypeptide encoded by an isolated nucleic acid molecule of the invention in a sample, the method comprising contacting the sample with an antibody that specifically binds to the encoded polypeptide.

The invention further pertains to a method of identifying an agent that alters expression of a MAP3K9 nucleic acid, comprising: contacting a solution containing a nucleic acid comprising the promoter region of the MAP3K9 gene operably linked to a reporter gene, with an agent to be tested; assessing the level of expression of the reporter gene in the presence of the agent; and comparing the level of expression of the reporter gene in the presence of the agent with a level of expression of the reporter gene in the absence of the agent; wherein if the level of expression of the reporter gene in the presence of the agent differs, by an amount that is statistically significant, from the level of expression in the absence of the agent, then the agent is an agent that alters expression of the MAP3K9 gene or nucleic acid. An agent identified by this method is also contemplated.

The invention additionally comprises a method of identifying an agent that alters expression of a MAP3K9 nucleic acid, comprising contacting a solution containing a nucleic acid of the invention or a derivative or fragment thereof, with an agent to be tested; comparing expression of the nucleic acid, derivative or fragment in the presence of the agent with expression of the nucleic acid, derivative or fragment in the absence of the agent; wherein if expression of the nucleic acid, derivative or fragment in the presence of the agent differs, by an amount that is statistically significant, from the expression in the absence of the agent, then the agent is an agent that alters expression of the MAP3K9 nucleic acid. In certain embodiments, the expression of the nucleic acid, derivative or fragment in the presence of the agent comprises expression of one or more splicing variants(s) that differ in kind or in quantity from the expression of one or more splicing variant(s) the absence of the agent. Agents identified by this method are also contemplated.

Representative agents that alter expression of a MAP3K9 nucleic acid contemplated by the invention include, for example, antisense nucleic acids to a MAP3K9 gene or nucleic acid; a MAP3K9 gene or nucleic acid; a MAP3K9 polypeptide; a MAP3K9 gene or nucleic acid receptor, or other receptor; a MAP3K9 binding agent; a peptidomimetic; a fusion protein; a prodrug thereof; an antibody; and a ribozyme. A method of altering expression of a MAP3K9 nucleic acid, comprising contacting a cell containing a nucleic acid with such an agent is also contemplated.

The invention further pertains to a method of identifying a polypeptide which interacts with a MAP3K9 polypeptide (e.g., a MAP3K9 polypeptide encoded by a nucleic acid of the invention, such as a nucleic acid comprising one or more polymorphism(s) described herein), comprising employing a yeast two-hybrid system using a first vector which comprises a nucleic acid encoding a DNA binding domain and a MAP3K9 polypeptide, splicing variant, or a fragment or derivative thereof, and a second vector which comprises a nucleic acid encoding a transcription activation domain and a nucleic acid encoding a test polypeptide. If transcriptional activation occurs in the yeast two-hybrid system, the test polypeptide is a polypeptide which interacts with a MAP3K9 polypeptide.

In certain methods of the invention, an asthma therapeutic agent is used. The asthma therapeutic agent can be an agent that alters (e.g., enhances or inhibits) MAP3K9 polypeptide activity and/or MAP3K9 nucleic acid expression, as described herein (e.g., a nucleic acid agonist or antagonist).

Asthma therapeutic agents can alter polypeptide activity or nucleic acid expression of a MAP3K9 nucleic acid by a variety of means, such as, for example, by providing additional polypeptide or upregulating the transcription or translation of the nucleic acid encoding the MAP3K9 polypeptide; by altering posttranslational processing of the MAP3K9 polypeptide; by altering transcription of splicing variants; or by interfering with polypeptide activity (e.g., by binding to the MAP3K9 polypeptide, or by binding to another polypeptide that interacts with MAP3K9, such as a MAP3K9 binding agent as described herein), by altering (e.g., downregulating) the expression, transcription or translation of a nucleic acid encoding MAP3K9; or by altering interaction among MAP3K9 and a MAP3K9 binding agent.

In a further embodiment, the invention relates to asthma therapeutic agent, such as an agent selected from the group consisting of: a MAP3K9 nucleic acid or fragment or derivative thereof; a polypeptide encoded by a MAP3K9 nucleic acid (e.g., encoded by a MAP3K9 nucleic acid having one or more polymorphism(s) such as those described herein); a MAP3K9 receptor; a MAP3K9 binding agent; a peptidomimetic; a fusion protein; a prodrug; an antibody; an agent that alters MAP3K9 gene or nucleic acid expression; an agent that alters activity of a polypeptide encoded by a MAP3K9 gene or nucleic acid; an agent that alters posttranscriptional processing of a polypeptide encoded by a MAP3K9 gene or nucleic acid; an agent that alters interaction of a MAP3K9 polypeptide with a MAP3K9 binding agent or receptor; an agent that alters transcription of splicing variants encoded by a MAP3K9 gene or nucleic acid; and ribozymes. The invention also relates to pharmaceutical compositions comprising at least one asthma therapeutic agent as described herein.

The present invention pertains to methods of diagnosing a susceptibility to asthma in an individual, comprising detecting a polymorphism in a MAP3K9 nucleic acid, wherein the presence of the polymorphism in the nucleic acid is indicative of a susceptibility to asthma. The invention additionally pertains to methods of diagnosing asthma in an individual, comprising detecting a polymorphism in a MAP3K9 nucleic acid, wherein the presence of the polymorphism in the nucleic acid is indicative of asthma. In one embodiment, in diagnosing asthma or susceptibility to asthma by detecting the presence of a polymorphism in a MAP3K9 nucleic acid, the presence of the polymorphism in the MAP3K9 nucleic acid can be indicated, for example, by the presence of one or more of the polymorphisms indicated in the Example Section.

In other embodiments, the invention relates to methods of diagnosing a susceptibility to asthma in an individual, comprising detecting an alteration in the expression or composition of a polypeptide encoded by a MAP3K9 nucleic acid in a test sample, in comparison with the expression or composition of a polypeptide encoded by a MAP3K9 nucleic acid in a control sample, wherein the presence of an alteration in expression or composition of the polypeptide in the test sample is indicative of a susceptibility to asthma. The invention additionally relates to a method of diagnosing asthma in an individual, comprising detecting an alteration in the expression or composition of a polypeptide encoded by a MAP3K9 nucleic acid in a test sample, in comparison with the expression or composition of a polypeptide encoded by MAP3K9 nucleic acid in a control sample, wherein the presence of an alteration in expression or composition of the polypeptide in the test sample is indicative of asthma.

The invention also pertains to a method of treating a disease or condition associated with a MAP3K9 polypeptide (e.g., asthma) in an individual, comprising administering a asthma therapeutic agent to the individual, in a therapeutically effective amount. In certain embodiments, the asthma therapeutic agent is a MAP3K9 agonist; in other embodiments, the asthma therapeutic agent is a MAP3K9 antagonist.

A transgenic animal comprising a nucleic acid selected from the group consisting of: an exogenous MAP3K9 gene or nucleic acid and a nucleic acid encoding a MAP3K9 polypeptide, is further contemplated by the invention.

In yet another embodiment, the invention relates to a method for assaying a sample for the presence of a MAP3K9 nucleic acid, comprising contacting the sample with a nucleic acid comprising a contiguous nucleotide sequence which is at least partially complementary to a part of the sequence of said MAP3K9 nucleic acid under conditions appropriate for hybridization, and assessing whether hybridization has occurred between a MAP3K9 nucleic acid and said nucleic acid comprising a contiguous nucleotide sequence which is at least partially complementary to a part of the sequence of said MAP3K9 nucleic acid; wherein if hybridization has occurred, a MAP3K9 nucleic acid is present in sample. In certain embodiments, the contiguous nucleotide sequence is completely complementary to a part of the sequence of said MAP3K9 nucleic acid. If desired, amplification of at least part of said MAP3K9 nucleic acid can be performed.

In certain other embodiments, the contiguous nucleotide sequence is 100 or fewer nucleotides in length and is either at least 80% identical to a contiguous sequence of nucleotides, at least 80% identical to the complement of a contiguous sequence of nucleotides; or capable of selectively hybridizing to said MAP3K9 nucleic acid.

In other embodiments, the invention relates to a reagent for assaying a sample for the presence of a MAP3K9 gene or nucleic acid, the reagent comprising a contiguous nucleotide sequence which is at least partially complementary to a part of the nucleic acid sequence of said MAP3K9 gene or nucleic acid; or comprising a contiguous nucleotide sequence which is completely complementary to a part of the nucleic acid sequence of said MAP3K9 gene or nucleic acid. Also contemplated by the invention is a reagent kit, e.g., for assaying a sample for the presence of a MAP3K9 nucleic acid, comprising (e.g., in separate containers) one or more labeled nucleic acids comprising a contiguous nucleotide sequence which is at least partially complementary to a part of the nucleic acid sequence of the MAP3K9 nucleic acid, and reagents for detection of said label. In certain embodiments, the labeled nucleic acid comprises a contiguous nucleotide sequence that is completely complementary to a part of the nucleotide sequence of said MAP3K9 gene or nucleic acid. In other embodiments, the labeled nucleic acid can comprise a contiguous nucleotide sequence which is at least partially complementary to a part of the nucleotide sequence of said MAP3K9 gene or nucleic acid, and which is capable of acting as a primer for said MAP3K9 nucleic acid when maintained under conditions for primer extension.

The invention also provides for the use of a nucleic acid which is 100 or fewer nucleotides in length and which is either: a) at least 80% identical to a contiguous sequence of nucleotides; b) at least 80% identical to the complement of a contiguous sequence of nucleotides; or c) capable of selectively hybridizing to said MAP3K9 nucleic acid, for assaying a sample for the presence of a MAP3K9 nucleic acid.

In yet another embodiment, the use of a first nucleic acid which is 100 or fewer nucleotides in length and which is either: a) at least 80% identical to a contiguous sequence of nucleotides; b) at least 80% identical to the complement of a contiguous sequence of nucleotides; or c) capable of selectively hybridizing to said MAP3K9 nucleic acid; for assaying a sample for the presence of a MAP3K9 gene or nucleic acid that has at least one nucleotide difference from the first nucleic acid (e.g., a SNP as set forth herein), such as for diagnosing a susceptibility to a disease or condition associated with a MAP3K9.

The invention also pertains to the use of a MLK family kinase inhibitor for the manufacture of a medicament for treatment for asthma in an individual, wherein the individual has at least one risk factor selected from the group consisting of: an at-risk haplotype for asthma; an at-risk haplotype in the MAP3K9 gene; a polymorphism in a MAP3K9 nucleic acid; dysregulation of MAP3K9 mRNA expression, dysregulation of a MAP3K9 mRNA isoform; increased MLK1 protein expression; increased MLK1 biochemical activity; and increased MKL1 protein isoform expression. In certain embodiments, the MLK family kinase inhibitor is a MLK1 inhibitor, for example CEP-1347 (Formula III) and its optically pure stereoisomers, mixtures of stereoisomers and salts or an indolocarbazole derivative and its optically pure stereoisomers, mixtures of stereoisomers and salts.

Also contemplated by the present invention is use of a first nucleic acid molecule for diagnosing asthma or a susceptibility to asthma in a sample from an individual to be diagnosed, comprising detecting in the sample the presence or absence of a second nucleic acid molecule of at least one marker of an at-risk haplotype associated with the MAP3K9 gene selected from the group consisting of: haplotype 1, 2, 3, 4, 5, 6, 7, 8, 9 and 10 of Table 1 and combinations thereof by contact with the first nucleic acid, wherein the presence of one or more markers is indicative of asthma or a susceptibility to asthma. The presence or absence of the marker can be accomplished by enzymatic amplification of nucleic acids, electrophoretic analysis, restriction fragment length polymorphism analysis or sequence analysis.

### BRIEF DESCRIPTION OF THE DRAWINGS

The foregoing and other objects, features and advantages of the invention will be apparent from the following more particular description of preferred aspects of the invention, as illustrated in the accompanying drawings.

The patent or application file contains at least one drawing executed in color. Copies of this patent or patent application publication with color drawings will be provided by the Office upon request and payment of the necessary fee.

FIG. 1 is an illustration of the JNK signaling cascade. 3

FIG. 2 shows examples of asthma pedigrees used in the linkage analysis. Unaffected siblings of patients are not shown and sex indicators have been shuffled for some individuals in the top two generations to protect privacy. The darkened squares and circles represent affected men and women, respectively. The slashed symbols represent deceased individuals.

FIG. 3 shows multipoint allele-sharing lod score of chromosome 14. A framework genome scan is shown by a dotted line. A fine mapping lod score of 4.00 was detected within the peak region after adding 34 microsatellite markers to obtain a marker density of less than 0.2 cM, using deCODE's high-density genetic map to determine genetic distances. The multipoint lod score is on the y-axis and centimorgan distance from the p-terminus of the chromosome is on the x-axis.

FIG. 4 shows mean age, gender, smoking history, % patients with positive skin tests, mean total IgE level, asthma severity level and lung function values expressed as % predicted forced expiratory volume in one second (% FEV1), forced expiratory volume in one second/forced vital capacity ratio (FEV1/FVC) and % of patients requiring ≦2.0 mg/ml and ≦8 mg/ml of methacholine, respectively, to produce 20% drop in FEVL (PC₂₀) for the asthma study population.

FIG. 5 shows a map of the MAP3K9 gene with SNPs and microsatellites.

FIGS. 6.A and 6.B show the linkage disequilibrium (LD) plot for chr 14q24.2-3 region with all markers present. Mapping of the linkage disequilibrium blocks using multiple microsatellite and SNP markers (x- and y-axis), covering the one lod drop on chromosome 14q24.2-3 (FIG. 6.A) and the 3 LD blocks (FIG. 6.B) that show the strongest association to asthma and include the MAP3K9gene. The d-dimer plot is shown above the transverse line (i.e., LD of any two markers next to each other) and the corresponding p-values are shown below the line. The significance of the d-dimer and the p values is reflected in the difference in black and white intensities (vertical axis to the right). FIG. 6.B shows a narrowly focused section of the linkage disequilibrium plot of FIG. 6.A in the region where the markers comprising the haplotypes reside.

FIGS. 7A to 7T show the nucleic acid sequence for MAP3K9 with coding regions (SEQ ID NO: 1). The upper case letters indicate the coding regions (exons).

FIG. 8 is the amino acid sequence of MAP3K9 (SEQ ID NO: 2).

FIG. 9 is a graph showing the MAP3K9 expression in asthma airway tissue using RT-PCR. OLD: Surgical resection of non-cancer tissue from individuals; OLD-1 and OLD-2, with obstructive lung disease. CO: Control Lung (surgical resection of non-cancer tissue from 2 individuals; CO-1 and CO-2). The results show a markedly enhance expression of Map3K9 expression in asthma compared to control airway tissue.

FIG. 10 is a graph showing MAP3K9 expression in PBM cells from asthma patients vs. control subjects. The results indicate a significant enhanced expression in PBM cells from asthma patients for variant b comparable to lung data.

FIGS. 11A-11O shows the mRNA and amino acid sequences for the splice variants a-e.

FIGS. 12A to 12Z43 are a table of microsatellite and SNP markers with the forward and reverse primer sequences. Also provided are the amplimer sequences and the nucleic acid start and end positions.

### DETAILED DESCRIPTION OF THE INVENTION

Extensive genealogical information for a population with population-based lists of patients has been combined with powerful genome sharing methods to map the first major locus in asthma with genome-wide significance. A genome-wide scan on patients, related within 6 meiotic events, diagnosed with asthma and their unaffected relatives has been completed. Locus AS1 on chromosome 14q24 has been identified through linkage studies to be associated with asthma. This locus does not correspond to known susceptibility loci for asthma, and represents the first mapping of a gene for asthma on chromosome 14q24. Until now there have been no known linkage studies of asthma in humans showing connection to this region of the chromosome. Based on the linkage studies conducted, Applicants have discovered a direct relationship between the genome wide significant (GWS) AS1 locus and asthma.

Linkage and association studies have identified a gene MAP3K9, that is located on 14q24.2-3 and is in the middle of a region where there is a significant 4-marker haplotype that overlays the MAP3K9 gene and is present in up to 12.8% of patients and 5% in controls (Relative Risk 2.81). This haplotype is the most common of ten overlapping microsatellite haplotypes that extend over a 170 kb. After typing additional SNPs, a 46 kb long haplotype was uncovered consisting of four SNPs, extending over exons 1 and 2 and the promoter region of MAP3K9. This haplotype is found in 15.7% of patients vs 6.8% of controls and confers a relative risk of 2.56. See Tables 1 and 2 in the Examples Section. All the patients studied have physician-diagnosed asthma (⅔ of who also have atopy as determined by positive skin tests and/or elevated IgE). All ten genetic marker haplotypes are shown in Table 1 and their p-values (p-val) and relative risk ratios (r) are shown in Table 2. Initially the MAP3K9 gene was isolated from a human epithelial tumor cell line. Expression of MAP3K9 has been found in lung tumor cell and different cell lines from the immune system as well as in smooth muscle cells.

MAP3K9 is a part of Mitogen-Activated Protein Kinase (MAPK) signal transduction pathways, which are among the most widespread mechanisms of eukaryotic cell regulation. In all eukaryotic cells there are multiple MAPK pathways, each reacting to different stimuli. The regulation of MAP3Ks represents an entry point into the MAPK pathways and is therefore complex.

Several kinases have been targeted therapeutically in various diseases and their function shown to be effectively modulated using small molecules. An example is the development of new small molecule inhibitors of p38 kinase that are being considered a potential new therapy for asthma. The MAP3K9 kinase is a gene that overlays the center of a haplotype that is almost three times more common in patients than in controls (i.e., RR 2.8); no asthma gene has been isolated today that carries a higher risk than this gene. The gene is in the pathway of cell signaling that involves mitogenic and second messenger activities (including IP3 regulation). Thus, this gene is a strong therapeutic target candidate for the development of new small molecule therapy for patients with asthma.

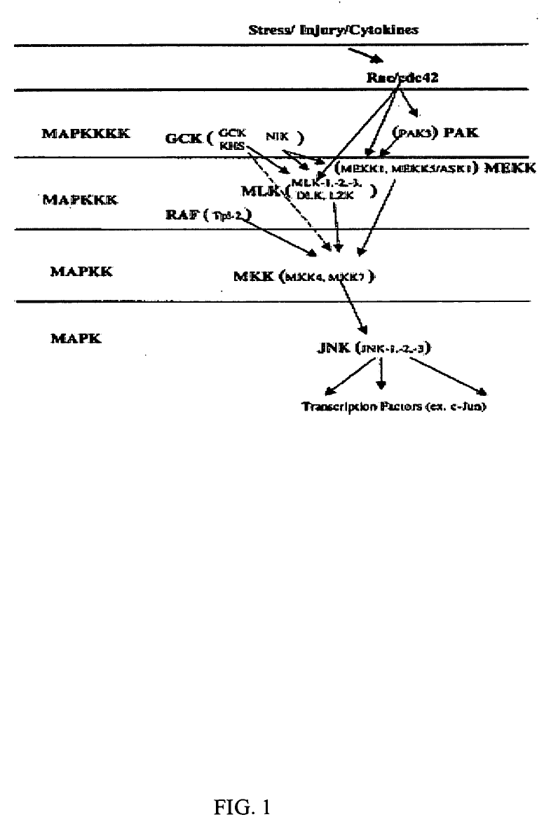

MAP3K9 is a member of the Mixed Lineage Kinase (MLK) family. The kinase domain of MLK family kinases has amino acid sequence similarity to both the tyrosine-specific and the serine/threonine-specific kinase classes although MAP3K9 is a serine/threonine kinase. Known serine/threonine phosphorylation substrates of MLK family members are the kinases MKK7 or MKK4. MLK family kinases, MKK7 and MKK4 are all known members of the JNK signalling cascade (see FIG. 1). Within the JNK signalling cascade there are three tiers of kinases linking stimuli such as cellular stress, injury or cytokines through the JNKs to transcriptional regulation via phosphorylation of c-Jun and related transcription factors (JunB & JunD).

The JNKs and a substrate of the JNKs, c-Jun have been implicated in the positive regulatory control of cell death or apoptosis. This could relate to asthma through two fundamental processes: 1) dysregulation of immune function through inappropriate cell death or lack thereof; and 2) inappropriate vascular and airway smooth muscle cell hyperplasia and consequent airway thickening increasing the risk or severity of asthma. While the MAP3K9 gene itself has never been associated with asthma, the JNK pathway has been implicated in the following processes that relate to various aspects of asthma:
1. Nitric oxide-induced AP-1 activation in human bronchial epithelial cells T-helper type 2 cell differentiation and production of the pro-inflammatory cytokines IFN-gamma and TGF-beta;
2. Allergen-induced airway inflammation in mice (this is reduced in Jun-B deficient mice); and
3. LPS induced IL-10 and IL-13 production by mast cells relating to airway inflammation in asthma.

The MLK-1 & the JNK pathway have been shown to regulate TNFα & IL-1b secretion, both of which are important cytokines in asthma. Moreover, the TH2-type cytokinese, IL10, IL13 and IL5, all of which are effective modulators of airway smooth muscle (ASM) contractility and relaxation, exert their effects on airway hyperresponsiveness, at least in part, through the induced expression and autocrine action of IL1beta (Hakonarson and Grunstein, Respir Physiol Neurobiol. September 16; 137(2-3):263-76 (2003); Nakae S et al., Int Immunol. April ;15(4):483-90 (2003)). Apart from regulation of IL-1b and TNFα secretion, the MLK-1 signaling pathway, through JNK and c-jun, is involved in the regulation of various other pro-inflammatory cytokines, including IL6, IL8 as well as various chemokines and TH1-type cytokines (IL2, IL12, IFNg) through its regulation of AP-1 transcription factor activity. IL-1b and TNFα have been shown to be critically involved in the local regulation of airway inflammation (Wuyts et al., Respir Med. July;97(7):811-7 (2003)). In addition, IL1b and to a lesser extend, TNFα are key regulators of ASM contractility and relaxation, two of the cardinal phenotypic features of asthma (Hakonarson et al. Mechanism of cytokine-induced modulation of beta-adrenoceptor responsiveness in airway smooth muscle. (Hakonarson et al., J Clin Invest. June 1;97(11):2593-600 (1996); Autocrine role of interleukin 1beta in altered responsiveness of atopic asthmatic sensitized airway smooth muscle. (J Clin Invest. January 1 ;99(1): 117-24 (1997)). Both IL1b and TNFα are potent mitogens in ASM and mucus glands (Page et al., Front Biosci. 5: 258-267, (2000); Stylianou et al., Int. J. Biochem Cell Biol. October;30(10):1075-9 (1998)) and thereby account for, at least in part, the increased ASM muscle mass and mucus hypersecretion, the other principal features of the asthma phenotype. Therefore, an inhibitor of MLK-1 such as the compound, CEP-1347, that has been shown to potently inhibit the release of TNF & IL-1 secretion in various cellular systems, would be anticipated to block asthma.

Chemical inhibitors of MLK family kinases have been described (Maroney et al., JBC, 276(27): 25302-25308 (2001)). For example, CEP-1347 (formula III) directly inhibits MLK family kinases including MLK1, MLK2 and MLK3. Inhibitory potency defined as the concentration of CEP-1347 needed to inhibit MLK kinase activity in a standardized biochemical assay is 38 nM±17 nM, 51 nM±9 nM and 23 nM±0.1 nM for MLK1, MLK2 and MLK3 respectively. CEP-1347 also effectively inhibits the activity of MLK kinases within intact cells with inhibitory potencies of 61 nM±11 nM, 82 nM±10 nM and 39 nM±3 nM for MLK1, MLK2 and MLK3, respectively. The kinetics of CEP-1347 inhibition of MLK kinases is consistent with a mode of action competitive with the binding of adenosine triphosphate in the MLK active site.

In addition, CEP-1347 has been shown to inhibit the production of TNFα and IL-1β by cultured cells under standard in vitro pharmacological testing procedures (see WO 97/49406). Also in animals, CEP-1347 reduces production of TNFα and IL-1β by mice after challenge with lipopolysaccharide (LPS) and provides protection from LPS-induced death. Since IL1b and TNFα are key regulators of ASM contractility (i.e., bronchial hyperresponsiveness) and relaxation, two of the cardinal phenotypic features of asthma and are the principal cytokines responsible for ASM and epithelial gland hypertrophy and hyperplacia as well as having profound autocrine effects that promote local airway inflammation, inhibition of IL-1b and TNFα would be anticipated to benefit asthma. Thus, CEP-1347 possesses pharmaceutic properties indicative of its potential benefit for the treatment of human diseases including asthma resulting from MAP3K9 gene dysregulation and consequent dysregulated production of MLK1.

Target At-Risk Populations

Target populations for the methods described herein, include individuals having an at-risk factor in a MAP3K9 gene haplotype or a polymorphism in the MAP3K9 gene. These at-risk individuals with the MAP3K9 DNA risk haplotype are a subset of all patients with asthma. Risk populations also include individuals with dysregulation of MAP3K9 gene transcription and dysregulation of a MAP3K9 mRNA isoform for example, an increase in RNA transcripts of MLK1 protein or an isoform of the protein. At-risk populations can have increased MLK1 biochemical activity, increased levels of MLK1 protein or a particular MLK1 protein isoform level can be increased. Thus, at-risk populations with MAP3K9 gene associated asthma can have differences with DNA sequences, RNA regulation and protein expression. These underlying genetic and protein differences can be manipulated in the type and extent of treatment provided.

Isolation and identification of target populations for treatment of individuals are advantageous for many reasons. For example, it can be possible to identify individuals in a specific at-risk population that respond to a specific treatment, such as treatment with one of the compounds disclosed herein. Samples from individuals with asthma can be tested in a diagnostic assay such as those described herein to assist in identifying the underlying genetic cause of the disease.

Direct efficient treatment of the subset of the population with an underlying genetic cause of asthma is possible utilizing the diagnositic and treatments described in the instant application. Knowledge of the underlying cause is useful in identifying an individual likely to be a responder to a particular treatment designed for the underlying cause from an individual that is a non-responder to this treatment. For example, if an asthmatic individual is diagnosed as a carrier of a DNA based haplotype or variant of the isoform of MAP3K9 protein, this individual would be more likely to repond to one of the compounds described herein that interfere with the perturbed JNK pathway. Thus, an individual identified by diagnosis as a target at-risk population can have a treatment tailored for the specific diagnosis, thereby reducing possible side effects or other deleterious reactions an individual can have with conventional treatment.

Generally, conventional treatments typically correct only the symptoms associated with the disease and do not prevent, delay or arrest the progression of the disease. Therefore, specific diagnosis of the target at-risk population and subsequent treatments as described herein, allows the patient to be treated to not only reduce the symptoms associated with the disease but also hold the progression of the disease by remodling the underlying genetic problem.

Nucleic Acid Therapeutic Agents

In another aspect, a nucleic acid of the invention; a nucleic acid complementary to a nucleic acid of the invention; or a portion of such a nucleic acid (e.g., an oligonucleotide as described below); or a nucleic acid encoding a member of the MLK pathway (e.g., MAP3K9), can be used in “antisense” therapy, in which a nucleic acid (e.g., an oligonucleotide) which specifically hybridizes to the mRNA and/or genomic DNA of a nucleic acid is administered or generated in situ. The antisense nucleic acid that specifically hybridizes to the mRNA and/or DNA inhibits expression of the polypeptide encoded by that mRNA and/or DNA, e.g., by inhibiting translation and/or transcription. Binding of the antisense nucleic acid can be by conventional base pair complementarity, or, for example, in the case of binding to DNA duplexes, through specific interaction in the major groove of the double helix.

An antisense construct can be delivered, for example, as an expression plasmid as described above. When the plasmid is transcribed in the cell, it produces RNA that is complementary to a portion of the mRNA and/or DNA that encodes the polypeptide for the member of the MLK pathway (e.g., MAP3K9). Alternatively, the antisense construct can be an oligonucleotide probe that is generated ex vivo and introduced into cells; it then inhibits expression by hybridizing with the mRNA and/or genomic DNA of the polypeptide. In one aspect, the oligonucleotide probes are modified oligonucleotides that are resistant to endogenous nucleases, e.g., exonucleases and/or endonucleases, thereby rendering them stable in vivo. Exemplary nucleic acid molecules for use as antisense oligonucleotides are phosphoramidate, phosphothioate and methylphosphonate analogs of DNA (see also U.S. Pat. Nos. 5,176,996, 5,264,564 and 5,256,775). Additionally, general approaches to constructing oligomers useful in antisense therapy are also described, for example, by Van der Krol et al. (Biotechniques 6:958-976 (1988)); and Stein et al. (Cancer Res. 48:2659-2668 (1988)). With respect to antisense DNA, oligodeoxyribonucleotides derived from the translation initiation site are preferred.

To perform antisense therapy, oligonucleotides (mRNA, cDNA or DNA) are designed that are complementary to mRNA encoding the polypeptide. The antisense oligonucleotides bind to mRNA transcripts and prevent translation. Absolute complementarity, although preferred, is not required. A sequence “complementary” to a portion of an RNA, as referred to herein, indicates that a sequence has sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid, as described in detail above. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures.

The oligonucleotides used in antisense therapy can be DNA, RNA, or chimeric mixtures or derivatives or modified versions thereof, single-stranded or double-stranded. The oligonucleotides can be modified at the base moiety, sugar moiety, or phosphate backbone, for example, to improve stability of the molecule, hybridization, etc. The oligonucleotides can include other appended groups such as peptides (e.g. for targeting host cell receptors in vivo), or agents facilitating transport across the cell membrane (see, e.g., Letsinger et al., Proc. Natl. Acad. Sci. USA 86:6553-6556 (1989); Lemaitre et al., Proc. Natl. Acad. Sci. USA 84:648-652 (1987); PCT International Publication No. WO 88/09810) or the blood-brain barrier (see, e.g., PCT International Publication No. WO 89/10134), or hybridization-triggered cleavage agents (see, e.g., Krol et al., BioTechniques 6:958-976 (1988)) or intercalating agents. (See, e.g., Zon, Pharm. Res. 5: 539-549 (1988)). To this end, the oligonucleotide may be conjugated to another molecule (e.g., a peptide, hybridization triggered cross-linking agent, transport agent, hybridization-triggered cleavage agent).

The antisense molecules are delivered to cells that express the member of the MLK pathway in vivo. A number of methods can be used for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically. Alternatively, in a preferred aspect, a recombinant DNA construct is utilized in which the antisense oligonucleotide is placed under the control of a strong promoter (e.g., pol III or pol II). The use of such a construct to transfect target cells in the patient results in the transcription of sufficient amounts of single stranded RNAs that will form complementary base pairs with the endogenous transcripts and thereby prevent translation of the mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art and described above. For example, a plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systemically).

In another aspect of the invention, small double-stranded interfering RNA (RNA interference (RNAi)) can be used. RNAI is a post-transcription process, in which double-stranded RNA is introduced, and sequence-specific gene silencing results, though catalytic degradation of the targeted mRNA. See, e.g., Elbashir, S. M. et al., Nature 411:494-498 (2001); Lee, N. S., Nature Biotech. 19:500-505 (2002); Lee, S-K. et al., Nature Medicine 8(7):681-686 (2002) the entire teachings of these references are incorporated herein by reference.

RNAi is used routinely to investigate gene function in a high throughput fashion or to modulate gene expression in human diseases (Chi et al., PNAS,100 (1 1):6343-6346 (2003)).

Introduction of long double standed RNA leads to sequence-specific degradation of homologous gene transcripts. The long double stranded RNA is metabolized to small 21-23 nucleotide siRNA (small interfering RNA). The siRNA then binds to protein complex RISC (RNA-induced silencing complex) with dual function helicase. The helicase has RNAas activity and is able to unwind the RNA. The unwound si RNA allows an antisense strand to bind to a target. This results in sequence dependent degradation of cognate mRNA. Aside from endogenous RNAi, exogenous RNAi, chemically synthesized or recombinantly produced can also be used.

Using non-intronic portions of the MAP3K9 gene such as corresponding mRNA portions of SEQ ID NO: 1, target regions of the MAP3K9 gene that are accessible for RNAi are targeted and silenced. With this technique it is possible to conduct a RNAi gene walk of the nucleic acids of MAP3K9 and determine the amount of inhibition of the protein product. Thus, it is possible to design gene-specific therapeutics by directly targeting the mRNAs of Asthma-related MAP3K9 gene.

Endogenous expression of a member of the MLK pathway (e.g., MAP3K9) can also be reduced by inactivating or “knocking out” the gene or its promoter using targeted homologous recombination (e.g., see Smithies et al., Nature 317:230-234 (1985); Thomas & Capecchi, Cell 51:503-512 (1987); Thompson et al., Cell 5:313-321 (1989)). For example, an altered, non-functional gene of a member of the MLK pathway (or a completely unrelated DNA sequence) flanked by DNA homologous to the endogenous gene (either the coding regions or regulatory regions of the gene) can be used, with or without a selectable marker and/or a negative selectable marker, to transfect cells that express the gene in vivo. Insertion of the DNA construct, via targeted homologous recombination, results in inactivation of the gene. The recombinant DNA constructs can be directly administered or targeted to the required site in vivo using appropriate vectors, as described above. Alternatively, expression of non-altered genes can be increased using a similar method: targeted homologous recombination can be used to insert a DNA construct comprising a non-altered functional gene, or the complement thereof, or a portion thereof, in place of a gene in the cell, as described above. In another aspect, targeted homologous recombination can be used to insert a DNA construct comprising a nucleic acid that encodes a polypeptide variant that differs from that present in the cell.

Alternatively, endogenous expression of a member of the MLK pathway can be reduced by targeting deoxyribonucleotide sequences complementary to the regulatory region of the member of the MLK pathway (i.e., the promoter and/or enhancers) to form triple helical structures that prevent transcription of the gene in target cells in the body. (See generally, Helene, C., Anticancer Drug Des., 6(6):569-84 (1991); Helene, C. et al., Ann. N.Y Acad. Sci. 660:27-36 (1992); and Maher, L. J., Bioassays 14(12):807-15 (1992)). Likewise, the antisense constructs described herein, by antagonizing the normal biological activity of one of the members of the MLK pathway, can be used in the manipulation of tissue, e.g., tissue differentiation, both in vivo and for ex vivo tissue cultures. Furthermore, the anti-sense techniques (e.g., microinjection of antisense molecules, or transfection with plasmids whose transcripts are anti-sense with regard to a nucleic acid RNA or nucleic acid sequence) can be used to investigate the role of one or more members of the MLK pathway in the development of disease-related conditions. Such techniques can be utilized in cell culture, but can also be used in the creation of transgenic animals.

The therapeutic agents as described herein can be delivered in a composition, as described above, or by themselves. They can be administered systemically, or can be targeted to a particular tissue. The therapeutic agents can be produced by a variety of means, including chemical family kinase; recombinant production; in vivo production (e.g., a transgenic animal, such as U.S. Pat. No. 4,873,316 to Meade et al.), for example, and can be isolated using standard means such as those described herein. In addition, a combination of any of the above methods of treatment (e.g., administration of non-altered polypeptide in conjunction with antisense therapy targeting altered mRNA for a member of the MLK pathway; administration of a first splicing variant in conjunction with antisense therapy targeting a second splicing variant) can also be used.

The invention additionally pertains to use of such therapeutic agents, as described herein, for the manufacture of a medicament for the treatment of asthma and other MAPK39 gene linked respiratory diseases, e.g., using the methods described herein.

Methods of Therapy

As a result of these discoveries, methods are now available for the treatment of asthma, and other respiratory diseases including but not limited to: chronic obstructive pulmonary disease, chronic bronchitis and other MAP3K9 gene linked respiratory diseases and potentially also other inflammatory diseases (such as rheumatoid arthritis, psoriasis, multiple sclerosis and inflammatory bowel disease) with the use of MLK1 inhibitors, such as agents that inhibit MLK1 kinase activity and thus decrease cellular production of cytokines and other inflammatory mediators as a consequence of cell stimulation. The term “treatment” as used herein, refers not only to ameliorating symptoms associated with the disease or condition, but also preventing or delaying the onset of the disease or condition; preventing or delaying the occurrence of a second episode of the disease or condition; lessening the severity or frequency of symptoms of the disease or condition; and/or also lessening the need for concomitant therapy with other drugs that ameliorate symptoms associated with the disease or condition, e.g., corticosteroids. Methods are additionally available for assessing an individual's risk for developing asthma and/or other respiratory diseases. In one aspect, the individual to be treated is an individual who is susceptible (at an increased risk) for asthma, or for whom the severity of the disease or condition is associated with DNA at-risk haplotypes in the MAP3K9 gene, dysregulation of MAP3K9 mRNA expression, or increased amount of MLK1 protein and/or biochemical activity and/or an increased amount of a particular protein isoform or MLK1.

Methods of Treatment

The present invention encompasses methods of treatment (prophylactic and/or therapeutic, as described above) for asthma and other respiratory diseases in individuals, such as individuals in the target populations described above, as well as for other diseases and conditions associated with MAP3K9 or with other members of the MLK family kinase. Members of the “JNK pathway,” in particular members of the MLK family kinases as used herein, include other polypeptides (e.g., enzymes, receptors) and other molecules that are associated with JNK pathway signaling, including the transcription factors, c-jun, v-fos and AP-1, or production of an MLK protein, such as transcription of the MAP3K9 gene and production of MLK-1 protein and or the stability of MLK-1 protein. In particular, the invention relates to methods of treatment for asthma or a susceptibility to asthma, using an asthma therapeutic agent. An “asthma therapeutic agent” is an agent that alters (e.g., enhances or inhibits) MAP3K9 polypeptide activity and/or MAP3K9 nucleic acid expression, as described herein (e.g., an asthma nucleic acid antagonist). In certain aspects, the asthma therapeutic agent alters activity and/or nucleic acid expression of MAP3K9.

Asthma therapeutic agents can alter MAP3K9 polypeptide activity or nucleic acid expression by a variety of means, such as, for example, by decreasing MAP3K9 polypeptide or by downregulating the transcription or translation of the MAP3K9 nucleic acid; by altering posttranslational processing of the MAP3K9 polypeptide; by altering transcription of MAP3K9 splicing variants; or by interfering with MAP3K9 polypeptide activity (e.g., by binding to a MAP3K9 polypeptide), or by binding to another polypeptide that interacts with MAP3K9, by altering (e.g., downregulating) the expression, transcription or translation of a MAP3K9 nucleic acid, or by altering (e.g., agonizing or antagonizing) activity.

In particular, the invention relates to methods of treatment for asthma or susceptibility to asthma, for example: for individuals in an at-risk population such as those described; as well as methods of treatment for asthma or other respiratory diseases; methods for reducing risk of asthma; and/or for decreasing cellular cytokines through the use of agents that inhibit MLK kinase activity, for example CEP-1347, or compounds as encompassed by formula I and Tables A and B. The invention additionally pertains to use of one or more MKL inhibitors, as described herein, for the manufacture of a medicament for the treatment asthma and other respiratory diseases, e.g., using the methods described herein.

In the methods of the invention, the “asthma therapeutic agent” is a “MLK family inhibitor”. In one aspect, a “MLK family inhibitor” is an agent that inhibits MAP3K9 polypeptide activity and/or MAP3K9 nucleic acid expression, as described herein (e.g., a nucleic acid antagonist). In another aspect, a MLK family inhibitor is an agent that inhibits polypeptide activity and/or nucleic acid expression of multiple members of the MLK family kinases in the JNK pathway. In still another aspect, a MLK family inhibitor is an agent that alters activity or metabolism of a MLK kinase (e.g., an antagonist of a MLK kinase; an antagonist of a MLK kinase activator). In certain aspects, the MLK inhibitor alters activity and/or nucleic acid expression of MAP3K9.

MLK family kinase inhibitors can alter polypeptide activity or nucleic acid expression of a member of the JNK pathway, in a variety of means, such as, for example, by catalytically degrading, downregulating or interfering with the expression, transcription or translation of a nucleic acid encoding the member of the JNK pathway; by altering posttranslational processing of the polypeptide; by altering transcription of splicing variants; or by interfering with polypeptide activity (e.g., by binding to the polypeptide, or by binding to another polypeptide that interacts with that member of the JNK pathway, such as a MAP3K9 or MLK1 binding agent as described herein or some other binding agent of a member of the pathway); by altering interaction among two or more members of the MLK family kinases in the JNK pathway; or by antagonizing activity of a member of the JNK pathway.

Representative MLK family kinase inhibitors include the following:

agents that inhibit activity of a member of the MLK signalling pathway (e.g., MAP3K9 proteins, MLK1) for example, CEP-1347, and compounds represented by formula I and Tables A and B; agents that inhibit activity of activators of members of the MLK pathway, such as MLK1 activators, MLK2 activators, and MLK3 activators, or agents that bind to a MLK family kinases or otherwise affect the activity of the MLK signaling pathway (for example inhibitors of RAC1/Cdc42, MKK4 and MKK7), other agents that alter (e.g., inhibit or antagonize) expression of a member of the JNK pathway, such as MAP3K9 or MLK family kinase nucleic acid expression or polypeptide activity, or that regulate transcription of MAP3K9 splicing variants or (e.g., agents that affect which splicing variants are expressed, or that affect the amount of each splicing variant that is expressed);

polypeptides described herein and/or splicing variants encoded by the MAP3K9 nucleic acid or fragments or derivatives thereof;

other polypeptides (e.g., MAP3K9 activators); MAP3K9 binding agents; or agents that affect (e.g., increase) activity or decrease MAP3K9 polypeptide stability;

antibodies to MLKs, such as an antibody to an altered MAP3K9 polypeptide, or an antibody to a non-altered MAP3K9 polypeptide, or an antibody to a particular splicing variant encoded by a MAP3K9 nucleic acid as described above; for example MLK3 (A-20):SC 15068, Santa Cruz Biotechnology, Inc., Santa Cruz, Calif.) that can be delivered intracellularly to decrease activity or amount of MLK-1;

antisense nucleic acids or small double-stranded interfering RNA to nucleic acids encoding MAP3K9 or a MLK family kinase or other member of the JNK pathway, or fragments or derivatives thereof, including antisense nucleic acids to nucleic acids encoding the MAP3K9 or other MLK family kinase polypeptides, and vectors comprising such antisense nucleic acids (e.g., nucleic acid, cDNA, and/or mRNA, double-stranded interfering RNA, or a nucleic acid encoding an active fragment or derivative thereof, or an oligonucleotide; for example, the complement of one of SEQ ID Nos: 1, or a nucleic acid complementary to the nucleic acid encoding SEQ ID NO: 2, or fragments or derivatives thereof); other agents that alter (e.g., inhibit or antagonize) expression of a member of the JNK pathway, such as MAP3K9 or MLK family kinase nucleic acid expression or polypeptide activity, or that regulate transcription of MAP3K9 splicing variants or (e.g., agents that affect which splicing variants are expressed, or that affect the amount of each splicing variant that is expressed).

More than one MLK family kinase inhibitor can be used concurrently, if desired.

The therapy is designed to alter activity of a MAP3K9 polypeptide, a MLK family kinase or another member of the JNK pathway in an individual, such as by inhibiting or antagonizing activity. For example, a MLK family kinase inhibitor can be administered in order to decrease family kinase of MLKs within the individual, or to downregulate or decrease the expression or availability of the MAP3K9 nucleic acid or specific splicing variants of the MAP3K9 nucleic acid. Downregulation or decreasing expression or availability of a native MAP3K9 nucleic acid or of a particular splicing variant could minimize the expression or activity of a defective nucleic acid or the particular splicing variant and thereby minimize the impact of the defective nucleic acid or the particular splicing variant.

The MLK family kinase inhibitor(s) are administered in a therapeutically effective amount, i.e., an amount that is sufficient to treat the disease or condition, such as by ameliorating symptoms associated with the disease or condition, preventing or delaying the onset of the disease or condition, and/or also lessening the severity or frequency of symptoms of the disease or condition. The amount which will be therapeutically effective in the treatment of a particular individual's disease or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

In certain aspects of the invention, the MLK family kinase inhibitor agent is an agent that inhibits activity of MAP3K9. In certain methods of the invention, the agents set forth in Formula I, Tables A and B and Formula III (CEP-1347) can be used for prophylactic and/or therapeutic treatment for diseases and conditions associated with MAP3K9 or with other members of MLK family kinases or other members of the JNK pathway, or with increased MLK family kinase activity. In particular, they can be used for treatment for asthma or susceptibility to asthma, such as for individuals in an at-risk population as described above, (e.g., based on identified risk factors) and individual requirement treatment.

In one aspect of the invention, the MLK family kinase inhibitor is an inhibitor of MLK1 such as CEP-1347 (also known as KT7515, Cephalon, Inc., W. Chester, Pa.) its optically pure stereoisomers, mixtures of stereoisomers, salts, chemical derivatives, analogues, or other compounds inhibiting MAP3K9 that effectively decrease MLK family kinase when administered to humans.

The compounds contemplated as MLK family kinase inhibitors in the methods described herein can be represented by the following formula: Formula I 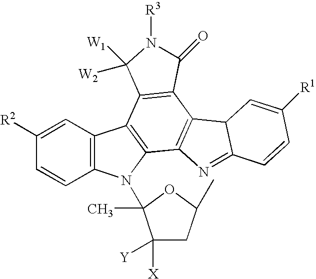
or a pharmaceutically acceptable salt thereof, wherein:
one of R¹ and R² is selected from the group consisting of:
a) —CO(CH₂)ⱼ R⁴, wherein j is 1 to 6, and R⁴ is selected from the group consisting of:
1) hydrogen and a halogen;
2) —NR⁵ R⁶, wherein R⁵ and R⁶ independently are hydrogen, substituted lower alkyl, unsubstituted lower alkyl, substituted aryl, unsubstituted aryl, substituted heteroaryl, unsubstituted heteroaryl, substituted aralkyl, unsubstituted aralkyl, lower alkylaminocarbonyl, or lower alkoxycarbonyl; or R⁵ and R⁶ are combined with a nitrogen atom to form a heterocyclic group;
3) N₃;
4) —SR²⁷, wherein R²⁷ is selected from the group consisting of:
i) hydrogen;
ii) substituted lower alkyl;
iii) unsubstituted lower alkyl;
iv) substituted aryl;
v) unsubstituted aryl;
vi) substituted heteroaryl;
vii) unsubstituted heteroaryl;
viii) substituted aralkyl;
ix) unsubstituted aralkyl;
x) thiazolinyl;
xi) —(CH₂)ₐ CO₂ R²⁸, wherein a is 1 or 2, and R²⁸ is selected from the group consisting of: hydrogen and lower alkyl; and
xii) —(CH₂)ₐ CONR⁵ R⁶; and
5) OR₂₉ (wherein R₂₉ is hydrogen, substituted lower alkyl, unsubstituted lower alkyl, or COR³⁰ (wherein R³⁰ is hydrogen, lower alkyl, substituted aryl, unsubstituted aryl, substituted heteroaryl, or unsubstituted heteroaryl));
b) —CH(OH)(CH₂)_{b} R^{4A}, wherein b is 1 to 6 and R^{4A} is hydrogen or the same as R⁴;
c) —(CH₂)_{d} CHR³¹ CO₂ R³² wherein d is O to 5, R31 is hydrogen, —CONR⁵ R⁶, or —CO₂ R³³ (wherein R³³ is hydrogen or lower alkyl), and R³² is hydrogen or lower alkyl;
d) —(CH2)_{d} CHR³¹ CONR⁵ R⁶;
e) —(CH₂)ₖ R⁷ wherein k is 2 to 6, and R⁷is halogen, CO₂R⁸ (wherein R⁸ is hydrogen, lower alkyl, substituted aryl, unsubstituted aryl, substituted heteroaryl, or unsubstituted heteroaryl), CONR⁵ R⁶, substituted aryl, unsubstituted aryl, substituted heteroaryl, unsubstituted heteroaryl, OR⁹ (wherein R⁹ is hydrogen, substituted lower alkyl, unsubstituted lower alkyl, acyl, substituted aryl, or unsubstituted aryl), SR^{27B} (wherein R^{27B} is the same as R²⁷), NR¹⁰ R¹¹ (wherein R¹⁰ and R¹¹ are the same as R⁵ and R⁶) or N₃;
f) —CH═CH(CH₂)ₘ R¹² wherein m is 0 to 4, and R¹² is hydrogen, lower alkyl, CO₂R^{8A} (wherein R^{8A} is the same as R⁸), —CONR⁵ R⁶, substituted aryl, unsubstituted aryl, substituted heteroaryl, unsubstituted heteroaryl, OR^{9A} (wherein R^{9A} is the same as R⁹), or NR^{10A} R^{11A} (wherein R^{10A} and R^{11A} are the same as R⁵ and R⁶);
g) —CH=C(CO₂R^{33A})₂, wherein R^{33A} is the same as R³³;
h) —C≡C(CH₂)ₙ R¹³, wherein n is 0 to 4, and R¹³ is the same as R¹²;
i) —CH₂OR⁴⁴, wherein R⁴⁴ is substituted lower alkyl;
and the other of R¹ or R² is selected from the group consisting of:
j) hydrogen, lower alkyl, halogen, acyl, nitro, NR¹⁴ R¹⁵ (wherein R¹⁴ or R¹⁵ is hydrogen or lower alkyl, and the other is hydrogen, lower alkyl, acyl, carbamoyl, lower alkylaminocarbonyl, substituted arylaminocarbonyl or unsubstituted arylaminocarbonyl);
k) —CH(SR³⁴)₂, wherein R³⁴ is lower alkyl or alkylene;
l) —CH₂R³⁵, wherein R³⁵ is OR³⁶ (wherein R³⁶ is tri-lower alkyl silyl in which the three lower alkyl groups are the same or different, or is the same as R²⁹), or SR³⁷ (wherein R³⁷ is the same as R²⁷);
m) —CO(CH2)_{q} R¹⁶, wherein q is 1 to 6, and R¹⁶ is the same as R⁴;
n) —CH(OH)(CH₂)ₑ R³⁸, wherein e is 1 to 6, and R³⁸ is the same as R^{4A};
o) —(CH₂)_{f}CHR³⁹ CO₂R⁴⁰, wherein f is 0 to 5, R³⁹ is the same as R³¹ and R⁴⁰ is the same as R³²;
p) —(CH₂)ᵣ R¹⁷, wherein r is 2 to 6, and R¹⁷ is the same as R⁷;
q) —CH═CH(CH₂)ₜR¹⁸, wherein t is 0 to 4, and R¹⁸ is the same as R¹²;
r) —CH═C(CO₂ R^{33B})₂, wherein R^{33B} is the same as R³³;
s) —C≡C(CH₂), R¹⁹, wherein u is 0 to 4, and R¹⁹ is the same as R¹³);
R³ is hydrogen, acyl, or lower alkyl;
X is selected from the group consisting of:
a) hydrogen;
b) formyl;
c) lower alkoxycarbonyl;
d) —CONR²⁰ R²¹, wherein:
R²⁰ and R²¹ independently are:
hydrogen;
lower alkyl;
—CH₂R²², wherein R²² is hydroxy, or
—NR²³ R²⁴ (wherein R²³ or R²⁴ is hydrogen or lower alkyl, and the other is hydrogen, lower alky, or the residue of an α-amino acid in which the hydroxy group of the carboxyl group is excluded, or R²³ and R²⁴ are combined with a nitrogen atom to form a heterocyclic group); and
e) —CH═N—R²⁵, wherein R²⁵ is hydroxy, lower alkoxy, amino, guanidino, or imidazolylamino;
Y is hydroxy, lower alkoxy, aralkyloxy, or acyloxy; or
X and Y combined represent, —X—Y—, ═O, —CH₂O(C═O)O—, —CH₂OC(═S)O—, —CH₂NR²⁶ C(═O)-(wherein R²⁶ is hydrogen or lower alkyl), —CH.₂ NHC(═S)O—, —CH₂OS(═O)O—, or —CH₂OC(CH₃)₂ O—; and
W¹ and W² are hydrogen, or W¹ and W² together represent oxygen.

The compounds represented by formula (I) are hereinafter referred to as Compound (I), and the same applies to the compounds of other formula numbers.

In the definitions of the groups in the formulas, lower alkyl means a straight-chain or branched alkyl group having 1 to 6 carbon atoms, such as methyl, ethyl, propyl, isopropyl, butyl, isobutyl, sec-butyl, tert-butyl, pentyl, isoamyl, neopentyl, 1-ethylpropyl and hexyl. The lower allyl moiety of lower alkoxy, lower alkoxycarbonyl, lower alkylaminocarbonyl and tri-lower alkylsilyl has the same meaning as lower alkyl defined above. The acyl moiety of the acyl and the acyloxy groups means a straight-chain or branched alkanoyl group having 1 to 6 carbon atoms, such as formyl, acetyl, propanoyl, butyryl, valeryl, pivaloyl and hexanoyl, an arylcarbonyl group described below, or a heteroarylcarbonyl group described below. The aryl moiety of the aryl, the arylcarbonyl and the arylaminocarbonyl groups means a group having 6 to 12 carbon atoms such as phenyl, biphenyl and naphthyl. The heteroaryl moiety of the heteroaryl and the heteroarylcarbonyl groups contain at least one hetero atom selected from O, S, and N, and include pyridyl, pyrimidyl, pyrrolyl, furyl thienyl, imidazolyl triazolyl, tetrazolyl, quinolyl, isoquinolyl benzoimidazolyl thiazolyl and benzothiazolyl. The aralkyl moiety of the aralkyl and the aralkyloxy groups means an aralkyl group having 7 to 15 carbon atoms, such as benzyl, phenethyl, benzhydryl and naphthylmethyl. The substituted lower alkyl group has 1 to 3 independently-selected substituents, such as hydroxy, lower alkoxy, carboxyl, lower alkoxycarbonyl, nitro, amino, mono- or di-lower alkylamino, dioxolane, dioxane, dithiolane, and dithione. The lower alkyl moiety of the substituted lower alkyl, and the lower allyl moiety of the lower alkoxy, the lower alkoxycarbonyl, and the mono- or di-lower alkylamino in the substituents of the substituted lower alkyl group have the same meaning as lower alkyl defined above. The substituted aryl, the substituted heteroaryl and the substituted aralkyl groups each has 1 to 3 independently-selected substituents, such as lower alkyl, hydroxy, lower alkoxy, carboxy, lower alkoxycarbonyl, nitro, amino, mono- or di-lower alkylamino, and halogen. The lower alkyl moiety of the lower alkyl the lower alkoxy, the lower alkoxycarbonyl, and the mono- or di-lower alkylamino groups among the substituents has the same meaning as lower alkyl defined above. The heterocyclic group formed with a nitrogen atom includes pyrrolidinyl, piperidinyl, piperidino, morpholinyl, morpholino, thiomorpholino, N-methylpiperazinyl, indolyl, and isoindolyl. The alpha.-amino acid groups include glycine, alanine, proline, glutamic acid and lysine, which may be in the L-form, the D-form or in the form of a racemate. Halogen includes fluorine, chlorine, bromine and iodine.

Preferably, one of R¹ and R² is selected from the group consisting of —CH₂)ₖ R⁷, —CH═CH(CH₂)ₘ R¹², —C≡C(CH₂)ₙ R¹³, —CO(CH₂)ⱼ SR²⁷, and —CH₂ OR⁴⁴ wherein R⁴⁴ is methoxymethyl, ethoxymethyl, or methoxyethyl; and the other of R1 and R² is selected from the group consisting of —(CH₂)ᵣ R¹⁷, —CH═CH(CH₂)ₜ R¹⁸, —C≡C(CH₂)ᵤ R¹⁹, NR¹⁴ R¹⁵, hydrogen, halogen, nitro, —CH₂O— (substituted or unsubstituted) lower alkyl, —CO(CH₂)_{q} SR²⁷, —CH₂R³⁵, —CH₂OH, and —CH₂SR³⁷ wherein R³⁷ is selected from the group consisting of lower alkyl, pyridyl, and benzimidazole.

Preferably, R³⁵ is OR³⁶ wherein R³⁶, preferably, is selected from the group consisting of methoxymethyl, ethoxymethyl, and methoxyethyl.

Preferably, R²⁷ is selected from the group consisting of substituted or unsubstituted lower alkyl, substituted or unsubstituted phenyl, pyridyl, pyrimidinyl, thiazole, and tetrazole.

Preferably, k and r, independently, are each 2, 3, or 4.

Preferably, j and q, independently, are 1 or 2.

Preferably, R⁷ and R¹⁷, independently, are selected from the group consisting of (1) CO₂ R⁸ and CO₂R^{8A}, where R⁸ and R^{8A}, independently, are hydrogen, methyl, ethyl, or phenyl; (2) phenyl, pyridyl, imidazolyl, thiazolyl, or tetrazolyl;(3) OR⁹ and OR^{9A} where R⁹ and R^{9A}, independently, are hydrogen, methyl, ethyl, phenyl, or acyl; (4) SR^{27B} where R^{27B} is selected from the group consisting of unsubstituted lower alkyl, 2-thiazoline, and pyridyl; and (5) NR¹⁰ R¹¹ and NR¹⁴ R¹⁵, where R¹⁰, R¹¹, R¹⁴, and R¹⁵, independently, are selected from the group consisting of hydrogen, methyl, ethyl, phenyl, carbamoyl, and lower alkylaminocarbonyl.

Preferably, m, n, t and u, independently, are 0 or 1.

Preferably, R¹², R¹³, R¹⁴, and R¹⁹, independently, are selected from the group consisting of hydrogen, methyl, ethyl, phenyl, pyridyl, imidazole, thiazole, tetrazole, CO₂R⁸, OR⁹, and NR¹⁰ R¹¹ where R⁸, R⁹, R¹⁰, and R¹¹ have the preferred vales s above.

Preferably, R³ is hydrogen or acetyl, most preferably hydrogen.

Preferably, X is hydroxymethyl or lower alkoxycarbonyl with methoxycarbonyl being particularly preferred.

Preferably, Y is hydroxy or acetyloxy, most preferably hydroxy.

Preferably, each W¹ and W² is hydrogen.

Most preferred are the actual substituent values shown on the compounds in Table 1, with Compounds 1-157 being especially preferred.

Examples of Compound (I) are shown in Table A and the intermediates are shown in Table B.

TABLE A

| Com- | | | | |
|---|---|---|---|---|
| pound | R¹ | R² | R³ | Y |
| 1 | CH═CHCO₂Me | CH═CHCO₂Me | H | OH |
| 2 | CH═CHCO₂Et | H | H | OH |
| 3 | CH═CHCO₂Et | CH═CHCO₂Et | H | OH |
| 4 | CH═CHCO₂Me | H | H | OH |
| 5 | CH═CH—C₆H₅ | H | H | OH |
| 6 | CH═CH—C₆H₅ | CH═CH—C₆H₅ | H | QH |
| 7 | CH═CH-2-Pyr | H | H | QH |
| 8 | CH═CH-2-Pyr | CH═CH-2-Pyr | H | OH |
| 9 | CH₂CH₂—C₆H₅ | CH₂CH₂—C₆H₅ | H | OH |
| 10 | CH₂CH₂—C₆H₅ | H | H | OH |
| 11 | CH₂CH₂-2-Pyr | CH₂CH₂-2-Pyr | H | OH |
| 12 | H | CH═CHCO₂Et | H | OH |
| 13 | H | CH═CH-2-Pyr | H | OH |
| 14 | H | CH₂CH₂-2-Pyr | H | OH |
| 15 | NO₂ | CH═CH-2-Pyr | Ac | OAc |
| 16 | NO₂ | CH═CH-2-Pyr | H | OH |
| 17 | NH₂ | CH₂CH₂-2-Pyr | Ac | OAc |
| 18 | NH₂ | CH₂CH₂-2-Pyr | H | OH |
| 19 | NHCONHEt | CH₂CH₂-2-Pyr | H | OH |
| 20 | C≡CCH₂NMe₂ | C≡CCH₂NMe₂ | H | OH |
| 21 | C≡CCH₂OMe | I | H | OH |
| 22 | C≡CCH₂OMe | C≡CCH₂OMe | H | OH |
| 23 | C≡CCH₂OH | C≡CCH₂OH | H | OH |
| 24 | COCH₂Cl | COCH₂Cl | Ac | OAc |
| 25 | COCH₂-1-Pip | COCH₂-1-Pip | H | OH |
| 26 | COCH₂CH₂Cl | H | Ac | OAc |
| 27 | COCH₂CH₂Cl | COCH₂CH₂Cl | Ac | OAc |
| 28 | COCH₂CH₂-1-Pip | H | H | OH |
| 29 | COCH₂CH₂-1-Pip | COCH₂CH₂-1-Pip | H | OH |
| 30 | COCH₂CH₂-1-Morph | COCH₂CH₂-1-Morph | H | OH |
| 31 | COCH₂-1-Morph | COCH₂-1-Morph | H | OH |
| 32 | COCH₂NMe₂ | COCH₂NMe₂ | H | OH |
| 33a | COCH₂Cl | H | Ac | OAc |
| 33b | H | COCH₂Cl | Ac | OAc |
| 34 | COCH₂NMe₂ | H | H | OH |
| 35 | COCH₂-1-THP | H | H | OH |
| 36a | COCH₂-1-Morph | H | Ac | OAc |
| 36b | H | COCH₂-1-Morph | Ac | OAc |
| 37a | COCH₂-1-Morph | H | H | OH |
| 37b | H | COCH₂-1-Morph | H | OH |
| 38 | COCH₂-1-THP | COCH₂-1-THP | H | OH |
| 39 | COCH₂-1-Pipz(4-Me) | COCH₂-1-Pipz(4-Me) | Ac | OAc |
| 40 | COCH₂-1-Pipz(4-Me) | COCH₂-1-Pipz(4-Me) | H | OH |
| 41 | H | COCH₂SEt | H | OH |
| 42a | COCH₂S-4-Pyr | H | H | OH |
| 42b | H | COCH₂S-4-Pyr | H | OH |
| 43 | COCH₂SMe | COCH₂SMe | H | OH |
| 44 | COCH₂SEt | COCH₂SEt | H | OH |
| 45 | COCH₂SCH₂Et | COCH₂SCH₂Et | H | OH |
| 46 | COCH₂S(CH₂)2 OH | COCH₂S(CH₂)2 OH | H | OH |
| 47 | COCH₂S-4-Pyr | COCH₂S-4-Pyr | H | OH |
| 48 | COCH₂S-2-Pyr | COCH₂S-2-Pyr | H | OH |
| 49 | COCH₂S-2-Pyrm | COCH₂S-2-Pyrm | H | OH |
| 50 | COCH₂S—C₆H₄(4- | COCH₂S—C₆H₄(4-OH) | H | OH |
| | OH) | | | |
| 51 | COCH₂S-2-Thiazl | COCH₂S-2-Thiazl | H | OH |
| 52 | COCH₂S-5-Tet(1-Me) | COCH₂S-5-Tet(1-Me) | H | OH |
| 53 | CO(CH₂)₂SMe | CO(CH₂)₂SMe | H | OH |
| 54 | CO(CH₂)₂OMe | CO(CH₂)₂OMe | H | OH |
| 55 | Br | CO(CH₂)₃H | H | OH |
| 56 | CO(CH₂)₄H | CO(CH₂)₄H | Ac | OAc |
| 57 | COCH₂Br | COCH₂Br | AC | OAc |
| 58 | CH(OH)Me | H | Ac | OAc |
| 59 | CH(OH)(CH₂)₂Cl | CH(OH)(CH₂)₂Cl | H | OH |
| 60 | CH(OH)CH₂-1-Pipz(4- | CH(OH)CH₂-1-Pipz(4-Me) | H | OH |
| | Me) | | | |
| 61 | C≡CCH₂NMe₂ | H | H | OH |
| 62 | Br | C≡CCH₂NMeBn | H | OH |
| 63 | CH═CHCH₂NMe₂ | CH═CHCH₂NMe₂ | Ac | OAc |
| 64 | CH═CHCH₂NMe₂ | CH═CHCH₂NMe₂ | H | OH |
| 65 | CH═CHEt | H | Ac | OAc |
| 66 | CH═CHEt | H | H | OH |
| 67 | CH═CHEt | I | Ac | OAc |
| 68 | CH═CHEt | CH═CHEt | H | OH |
| 69 | (CH₂)₂Cl | (CH₂)₂Cl | H | OH |
| 70a | (CH₂)₂I | (CH₂)₂I | H | OH |
| 70b | (CH₂)₂OCOH | (CH₂)₂OCOH | H | OH |
| 70c | (CH₂)₂OH | (CH₂)₂OH | H | OH |
| 71 | (CH₂)₂OCO-4-Pyr | (CH₂)₂OCO-4-Pyr | H | OH |
| 72a | CH₂CO₂Me | H | H | OH |
| 72b | CH₂CO₂Me | CH₂CO₂Me | H | OH |
| 73a | (CH₂)₃I | (CH₂)₃I | H | OH |
| 73b | (CH₂)₃OCOH | (CH₂)₃OCOH | H | OH |
| 73c | (CH₂)₃OH | (CH₂)₃OH | H | OH |
| 74 | (CH₂)₃OMe | (CH₂)₃OMe | H | OH |
| 75 | (CH₂)₂-1-Pip | (CH₂)₂-1-Pip | H | OH |
| 76 | (CH₂)₂-1-Morph | (CH₂)₂-1-Morph | H | OH |
| 77 | (CH₂)₂NEt₂ | (CH₂)₂NEt₂ | H | OH |
| 78 | (CH₂)₂NMe(CH₂)₂OH | (CH₂)₂NMe(CH₂)₂OH | H | OH |
| 79 | (CH₂)₂NHMe | (CH₂)₂NHMe | H | OH |
| 80 | (CH₂)₂NHCH₂C₆H₄(4- | (CH₂)₂NHCH₂C₆H₄(4- | H | OH |
| | MeO) | MeO) | | |
| 81 | (CH₂)₂N₃ | (CH₂)₂N₃ | H | OH |
| 82 | (CH₂)₃-1-Pip | (CH₂)₃-1-Pip | H | OH |
| 88 | (CH₂)₃-1-Morph | (CH₂)₃-1-Morph | H | OH |
| 84 | (CH₂)₃NEt₂ | (CH₂)₃NEt₂ | H | OH |
| 85 | (CH₂)₃NHCONHEt | (CH₂)₃NHCONHEt | H | OH |
| 86 | (CH₂)₃NHCO₂t-Bu | CH₂)₃NHCO₂t-Bu | H | OH |
| 87 | (CH₂)₂SMe | (CH₂)₂SMe | H | OH |
| 88 | (CH₂)₂SEt | (CH₂)₂SEt | H | OH |
| 89 | (CH₂)₂SCH₂CO₂Me | (CH₂)₂SCH₂CO₂Me | H | OH |
| 90 | (CH₂)₂S(CH₂)₂CO₂Et | (CH₂)₂S(CH₂)₂CO₂Et | H | OH |
| 91 | (CH₂)₂S—C₆H₄(4- | (CH₂)₂S—C₆H₄(4-OH) | H | OH |
| | OH) | | | |
| 92 | (CH₂)₂S-2-Thiazl | (CH₂)₂S-2-Thiazl | H | OH |
| 93 | (CH₂)₂S-4-Pyr | (CH₂)₂S-4-Pyr | H | OH |
| 94 | (CH₂)₂S-2-Pyr | (CH₂)₂S-2-Pyr | H | OH |
| 95 | (CH₂)₃SMe | (CH₂)₃SMe | H | OH |
| 96 | (CH₂)₃S-2- | (CH₂)₃S-2- | H | OH |
| | (Benz)Thiazole | (Benz)Thiazole | | |
| 97 | CH═CH-2-Pyr | CHO | Ac | OAc |
| 98 | CH═CH-2-Pyr | CH₂OH | Ac | OAc |
| 99 | CH═CH-2-Pyr | CH₂OH | H | OH |
| 100 | CH═CH-2-Pyr | CH₂OSiMe₂t-Bu | Ac | OAc |
| 101 | CH═CH-2-Pyr | CH₂OSiMe₂t-Bu | H | OH |
| 102 | CH═CH-2-Pyr | CH₂OMe | H | OH |
| 103 | CH═CH-2-Pyr | CH₂OEt | H | OH |
| 104 | CH═CH-2-Pyr | CH₂O(CH₂)₂NMe₂ | H | OH |
| 105 | CH═CH-2-Pyr | CH₂SEt | H | OH |
| 106 | CH═CH-2-Pyr | CH₂S(CH₂)₂NMe₂ | H | OH |
| 107 | CH═CH-2-Pyr | CH₂S-2-(Benz)Imid | H | OH |
| 108 | CH═CH-2-Pyr | CH₂S-2-Pyr | H | OH |
| 109 | CH═CH-2-Pyr | CH(SEt)₂ | Ac | OAc |
| 110 | CH═CH-2-Pyr | CH(SEt)₂ | H | OH |
| 111 | CHO | CH═CH-2-Pyr | Ac | OAc |
| 112 | CH₂OH | CH═CH-2-Pyr | Ac | OAc |
| 113 | CH₂OH | CH═CH-2-Pyr | H | OH |
| 114 | CH₂OSiMe₂t-Bu | CH═CH-2-Pyr | Ac | OAc |
| 115 | CH₂OSiMe₂t-Bu | CH═CH-2-Pyr | H | OH |
| 116 | CH₂OMe | CH═CH-2-Pyr | H | OH |
| 117 | CH₂OEt | CH═CH-2-Pyr | H | OH |
| 118 | CH₂SEt | CH═CH-2-Pyr | H | OH |
| 119 | CH₂S-2-Pyr | CH═CH-2-Pyr | H | OH |
| 120 | CH₂S-2-(Benz)Imid | CH═CH-2-Pyr | H | OH |
| 121 | CH═CHEt | CH═CH-2-Pyr | Ac | OAc |
| 122 | CH═CHEt | CH═CH-2-Pyr | H | OH |
| 123 | (CH₂)₂-2-Pyr | CH₂OSiMe₂t-Bu | Ac | OAc |
| 124 | (CH₂)₂-2-Pyr | CH₂OSiMe₂t-Bu | H | OH |
| 125a | (CH₂)₂-2-Pyr | CH₂OMe | Ac | OAc |
| 125b | (CH₂)₂-2-Pyr | CH₂OMe | H | OAc |
| 126 | (CH₂)₂-2-Pyr | CH₂OMe | H | OH |
| 127a | (CH₂)₂-2-Pyr | CH₂OEt | H | OH |
| 127b | (CH₂)₂-2-Pyr | CH₂OH | H | OH |
| 128 | (CH₂)₂-2-Pyr | CH₂S-2-Pyr | Ac | OAc |
| 129 | (CH₂)₂-2-Pyr | CH₂S-2-Pyr | H | OH |
| 130 | CH₂OSiMe₂t-Bu | (CH₂)₂-2-Pyr | Ac | OAc |
| 131 | CH₂OSiMe₂t-Bu | (CH₂)₂-2-Pyr | H | OH |
| 132 | CH₂OMe | (CH₂)₂-2-Pyr | H | OH |
| 133 | CH₂OEt | (CH₂)₂-2-Pyr | H | OH |
| 134 | CH₂SEt | (CH₂)₂-2-Pyr | H | OH |
| 135 | CH₂S(CH₂)₂NMe₂ | (CH₂)₂-2-Pyr | H | OH |
| 136 | CH₂S-2-Pyr | (CH₂)₂-2-Pyr | Ac | OAc |
| 137 | CH₂S-2-Pyr | (CH₂)₂-2-Pyr | H | OH |
| *138 | C≡CCH₂OMe | C≡CCH₂OMe | H | OH |
| 139 | CH₂CH₂CO₂Me | CH₂CH₂CO₂Me | H | OH |
| 140 | CH₂CH₂CO₂Et | CH₂CH₂CO₂Et | H | OH |
| 141 | Br | CH═CH-2-Pyr | Ac | OAc |
| 142 | Br | CH═CH-2-Pyr | H | OH |
| 143 | Br | CH₂CH₂-2-Pyr | H | OH |
| 144 | CH═CH-4-Pyr | CH═CH-4-Pyr | Ac | OAc |
| 145 | CH═CH-4-Pyr | CH═CH-4-Pyr | H | OH |
| 146 | CH₂CH₂-4-Pyr | CH₂CH₂-4-Pyr | H | OH |
| 147 | CH═CH-2-Imid | H | Ac | OAc |
| 148 | CH═CH-2-Imid | H | H | OH |
| 149 | CH₂CH₂-2-Imid | H | H | OH |
| 150 | CH═C(CO₂Me)₂ | CH═C(CO₂Me)₂ | Ac | OAc |
| 151 | CH₂CH(CO₂Me)₂ | CH₂CH(CO₂Me)₂ | Ac | OAc |
| 152 | CH₂CH(CO₂Me)₂ | CH₂CH(CO₂Me)₂ | H | OH |
| 153 | n-C₄H₉ | (CH₂)₂-2-Pyr | H | OH |
| 154 | CH₂OCH₂OMe | H | H | OH |
| 155 | CH₂OCH₂OMe | CH₂OCH₂OMe | H | OH |
| 156 | CH₂OCH₂OEt | CH₂OCH₂OEt | H | OH |
| 157 | CH₂O(CH₂)₂OMe | CH₂O(CH₂)₂OMe | H | OH |

Pyr = Pyridyl
Pip = Piperidine
Morph = Morpholine
THP = Tetrahydropyrrole
Pipz = Piperazine
Pyrm = Pyrimidine
Thiazl = Thiazoline
Tet = Tetrazole
Imid = Imidazole
(Benz)Thiazole = Benzothiazole
(Benz)Imid = Benzimidazole
*The CO₂CH₃ group is replaced with CH₂OH.

TABLE B

| Compound | R1 | R2 | R3 | Y |
|---|---|---|---|---|
| A | Br | H | Ac | OAc |
| B | Br | CHO | Ac | OAc |
| C | H | CHO | Ac | OAc |
| D | NO2 | H | Ac | OAc |
| E | NO2 | CHO | Ac | OAc |
| F | I | I | Ac | OAc |
| G | I | I | H | OH |
| H | I | H | Ac | OAc |
| I | Br | I | Ac | OAc |
| J | CHO | I | Ac | OAc |
| K | CH2OH | I | Ac | OAc |

See U.S. Pat. No. 6,306,849 and WO97/49406, incorporated herein by reference in their entirety.

The compounds are derivatives of the compound K-252a, represented by the following structure: Formula II. 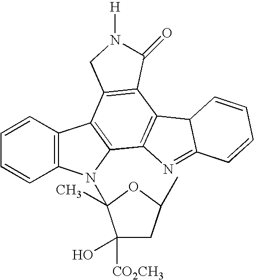
K-252a has an indolocarbazole skeleton as described in U.S. Pat. No. 4,555,402 and Japanese Published Unexamined Patent Application No. 41489/85. K-252a is a natural product indolocarbazole of the bacterium Nocardiosis species. The activity of these compounds can be demonstrated using the cultured pinal cord choline acetyltransferase (CHAT) assay.

Formula III represents, an ethylthiomethyl analog of K-252a, CEP-1347, that exhibited greater efficacy (250% of control) and potency (EC50=50 nM) than K-252a in spinal cord ChAT assays (Kaneko et al., J. Med. Chem. Jun 6;40(12):1863-9 (1997)). In certain aspects, the methods of the invention utilize CEP-1347 as the MLK1 inhibitor.

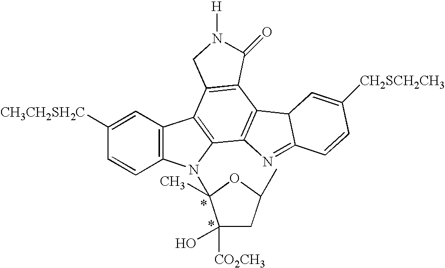

Formula III (CEP-1347) (* denotes chiral center)

Some disclosed compounds contain a chiral center. For example, in Formula III, the * denotes chiral centers. The presence of chiral centers in a molecule gives rise to stereoisomers. For example, a pair of optical isomers, referred to as “enantiomers”, exist for every chiral center in a molecule; and a pair of diastereomers exist for every chiral center in a compound having two or more chiral centers. Where the structural formulas do not explicitly depict stereochemistry, it is to be understood that these formulas encompass enantiomers free from the corresponding optical isomer, racemic mixtures, mixtures enriched in one enantiomer relative to its corresponding optical isomer, a diastereomer free of other diastereomers, a pair of diastereomers free from other diasteromeric pairs, mixtures of diasteromers, mixtures of diasteromeric pairs, mixtures of diasteromers in which one diastereomer is enriched relative to the other diastereomer(s) and mixtures of diasteromeric pairs in which one diastereomeric pair is enriched relative to the other diastereomeric pair(s).

Also compounds of Structural Formula IV (as described in WO 03/064428AI) are useful in the methods described herein as the MLK family kinase inhibitor. Structural Formula IV: 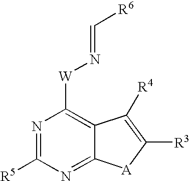
wherein A represents O or S;
W represents O, NH, NR¹;
R⁴ and R⁵ are independently selected from the group represented by hydrogen, halogen, cyano, nitro, C₁₋₆-alk(en/yn)yl, C₁₋₆-alk(en/yn)yloxy, C₁₋₆-alk(en/yn)yloxy-C₁₋₆-alk(en/yn)yl, C₁₋₆-alk(en/yn)ylsulfanyl, hydroxy, hydroxy-C₁₋₆-alk(en/yn)yl, halo-C₁₋₆-alk(en/yn)yl, halo-C₁₋₆-alk(en/yn)yloxy, C₃₋₈-cycloalk(en)yl, C₃₋₈-cycloalk(en)yl-C₁₋₆-alk(en/yn)yl, acyl, C₁₋₆-alk(en/yn)yloxycarbonyl, C₁₋₆-alk(en/yn)ylsulfonyl, —NR⁷R⁸ and R⁷R⁸N—C₁₋₆-alk(en/yn)yl-;
R³ represents hydrogen, halogen, C₁₋₆-alk(en/yn)yl, C₃₋₈-cycloalk(en/yn)yl, aryl, a heterocycle, hydroxy, hydroxy-C₁₋₆-alk(en/yn)yl, C₁₋₆-alk(en/yn)yloxy, C₁₋₆-alk(en/yn)yloxy-C₁₋₆-alk(en/yn)yl, C₃₋₈-cycloalk(en/yn)oxy, C₁₋₆-alk(en/yn)ylsulfanyl, acyl, R⁷R⁸N—C₁₋₆-alk(en/yn)yl or —NR⁷R⁸;
or R³ represents a group of the formula
—R⁹—Ar²
wherein R⁹ represents O, NH, NR¹, S, —CONR^{1′}—, —CO— or C₁₋₆-alkyl, C₂₋₆-alkenyl, which may optionally be substituted by OH, halogen, C₁₋₆-alkoxy or C₃₋₈-cycloalkyl;
R represents C₁₋₆-alk(en/yn)yl, C₃₋₈-cycloalk(en/yn)yl, C₃₋₈-cycloalk(en)yl-C₁₋₆-alk(en/yn)yl or Ar¹;
Ar¹ and Ar² are independently selected from the group represented by aryl, a heterocycle or a carbocycle all of which may be substituted one or more times by halogen, cyano, nitro, C₁₋₆-alk(en/yn)yl, C₁₋₆-alk(en/yn)yloxy, C₁₋₆-alk(en/yn)yloxy-C₁₋₆-alk(en/yn)yl, C₁₋₆-alk(en/yn)yloxy-C₁₋₆-alk(en/yn)yloxy-C₁₋₆-alk(en/yn)yl aryloxy-, aryl-C₁₋₆-alk(en/yn) yloxy, halo-C₁₋₆-alk(en/yn)yloxy, C₁₋₆-alk(en/yn)yl-sulfanyl, hydroxy, hydroxy-C₁₋₆-alk(en/yn)yl, halo-C₁₋₆-alk(en/yn)yl, cyano-C₁₋₆-alk(en/yn)yl, NR⁷R⁸, NR⁷R⁸-C₁₋₆-alk(en/yn)yl, C₃₋₈-cycloalk(en)yl, C₃₋₈-cycloalk(en)yl-C₁₋₆-alk(en/yn)yl, C₁₋₆-alk(en/yn)ylsulfonyl, aryl, acyl, C₁₋₆-alk(en/yn)yloxycarbonyl, C₁₋₆-alk(en/yn)yl-CONR^{1′}-C₁₋₆-alk(en/yn)yl, C₁₋₆-alk(en/yn)yl-CONR^{1′}—, —CONR⁷R⁸ or R⁷R⁸NCO—C₁₋₆-alk(en/yn)yl;
R⁷ and R⁸ are independently selected from the group represented by hydrogen and C₁₋₆-alk(en/yn)yl which may be further substituted by hydroxy, halogen, C₁₋₆-alkoxy, cyano, nitro, C₃₋₈-cycloalk(en)yl, C₃₋₈-cycloalk(en)yl-C₁₋₆-alk(en/yn)yl, aryl or a heterocycle; or
R⁷ and R⁸ together with the nitrogen to which they are attached form a 3-7-membered ring which optionally contains one or more further heteroatoms and may optionally be substituted by halogen, C₁₋₆-alk(en/yn)yl, hydroxy, hydroxy-C₁₋₆-alk(en/yn)yl or acyl;
the aryls may be further substituted by halogen, cyano, nitro, C₁₋₆-alk(en/yn)yl, C₁₋₆-alk(en/yn)yloxy, C₁₋₆-alk(en/yn)ylsulfanyl, hydroxy, hydroxy-C₁₋₆-alk(en/yn)yl, halo-C₁₋₆-alk(en/yn)yl, halo-C₁₋₆-alk(en/yn)yloxy, C₃₋₈-cycloalk(en)yl, C₃₋₈-cycloalk(en)yl-C₁₋₆-alk(en/yn)yl, acyl, C₁₋₆-alk(en/yn)yloxycarbonyl, C₁₋₆-alk(en/yn)ylsulfonyl, or —NR^{7′}R^{8′} wherein —NR^{7′}R^{8′} is as defined for —NR⁷R⁸ above provided that any aryl substituent on —NR^{7′}R^{8′} is not further substituted;
and R¹ and R^{1′} are independently selected from the group represented by C₁₋₆-alk(en/yn)yl, C₃₋₈-cycloalk(en)yl, aryl, hydroxy-C₁₋₆-alk(en/yn)yl, C₃₋₈-cycloalk(en)yl-C₁₋₆-alk(en/yn)yl and acyl;
or a pharmaceutically acceptable salt thereof.

The term “alkyl” refers to a monovalent group derived from a straight or branched chain saturated hydrocarbon by the removal of a single hydrogen atom. Alkyl groups are exemplified by methyl, ethyl, n- and iso-propyl, n-, sec-, iso- and tert-butyl, and the like. The term “hydroxyalkyl” represents an alkyl group, as defined above, substituted by one to three hydroxyl groups with the proviso that no more than one hydroxy group may be attached to a single carbon atom of the alkyl group. The term “alkylamino” refers to a group having the structure —NHR′ wherein R′ is alkyl, as previously defined, examples of alkylamino include methylamino, ethylamino, iso-propylamino and the like. The term “alkanoyl” represents an alkyl group, as defined above, attached to the parent molecular moiety through a carbonyl group. Alkanoyl groups are exemplified by formyl, acetyl, propionyl, butanoyl and the like. The term “alkanoylamino” refers to an alkanoyl group, as previously defined, attached to the parent molecular moiety through a nitrogen atom. Examples of alkanoylamino include formamido, acetamido, and the like. The term “N-alkanoyl-N-alkylamino” refers to an alkanoyl group, as previously defined, attached to the parent molecular moiety through an aminoalkyl group. Examples of N-alkanoyl-N-alkylamino include N-methylformamido, N-methyl-acetamido, and the like. The terms “alkoxy” or “alkoxyl” denote an alkyl group, as defined above, attached to the parent molecular moiety through an oxygen atom. Representative alkoxy groups include methoxyl, ethoxyl, propoxyl, butoxyl, and the like. The term “alkoxyalkoxyl” refers to an alkyl group, as defined above, attached through an oxygen to an alkyl group, as defined above, attached in turn through an oxygen to the parent molecular moiety. Examples of alkoxyalkoxyl include methoxymethoxyl, methoxyethyoxyl, ethoxyethoxyl and the like. The term “alkoxyalkyl” refers to an alkoxy group, as defined above, attached through an alkylene group to the parent molecular moiety. The term “alkoxycarbonyl” represents an ester group; i.e., an alkoxy group, attached to the parent molecular moiety through a carbonyl group such as methoxycarbonyl, ethoxycarbonyl, and the like. The term “alkenyl” denotes a monovalent group derived from a hydrocarbon containing at least one carbon-carbon double bond by the removal of a single hydrogen atom. Alkenyl groups include, for example, ethenyl, propenyl, butenyl, 1-methyl-2-buten-1-yl and the like. The term “alkylene” denotes a divalent group derived from a straight or branched chain saturated hydrocarbon by the removal of two hydrogen atoms, for example methylene, 1,2-ethylene, 1,1-ethylene, 1,3-propylene, 2,2-dimethylpropylene, and the like. The term “alkenylene” denotes a divalent group derived from a straight or branched chain hydrocarbon containing at least one carbon-carbon double bond. Examples of alkenylene include —CH═CH—, —CH₂CH═CH—, —C(CH₃)═CH—, —CH₂CH═CHCH₂—, and the like. The term “cycloalkylene” refers to a divalent group derived from a saturated carbocyclic hydrocarbon by the removal of two hydrogen atoms, for example cyclopentylene, cyclohexylene, and the like. The term “cycloalkyl” denotes a monovalent group derived from a monocyclic or bicyclic saturated carbocyclic ring compound by the removal of a single hydrogen atom. Examples include cyclopropyl, cyclobutyl, cyclopentyl, cyclohexyl, bicyclo[2.2.1]heptanyl, and bicyclo[2.2.2]octanyl. The term “alkynylene” refers to a divalent group derived by the removal of two hydrogen atoms from a straight or branched chain acyclic hydrocarbon group containing a carbon-carbon triple bond. Examples of alkynylene include —CH s═CH—, —CH≡CH—CH₂—, —CH≡CH—CH(CH₃)—, and the like. The term “carbocyclic aryl” denotes a monovalent carbocyclic ring group derived by the removal of a single hydrogen atom from a monocyclic or bicyclic fused or non-fused ring system obeying the “4n+2 p electron” or Huckel aromaticity rule. Examples of carbocyclic aryl groups include phenyl, 1- and 2-naphthyl, biphenylyl, fluorenyl, and the like. The term “(carbocyclic aryl)alkyl” refers to a carbocyclic aryl ring group as defined above, attached to the parent molecular moiety through an alkylene group. Representative (carbocyclic aryl)alkyl groups include phenylmethyl, phenylethyl, phenylpropyl, 1-naphthylmethyl, and the like. The term “halo or halogen” denotes fluorine, chlorine, bromine or iodine. The term “haloalkyl” denotes an alkyl group, as defined above, having one, two, or three halogen atoms attached thereto and is exemplified by such groups as chloromethyl, bromoethyl, trifluoromethyl, and the like. The term “hydroxyalkyl” represents an alkyl group, as defined above, substituted by one to three hydroxyl groups with the proviso that no more than one hydroxy group may be attached to a single carbon atom of the alkyl group. The term “phenoxy” refers to a phenyl group attached to the parent molecular moiety through an oxygen atom. The term “phenylthio” refers to a phenyl group attached to the parent molecular moiety through a sulfur atom. The term “pyridyloxy” refers to a pyridyl group attached to the parent molecular moiety through an oxygen atom. The terms “heteroaryl” or “heterocyclic aryl” as used herein refers to substituted or unsubstituted 5- or 6-membered ring aromatic groups containing one oxygen atom, one, two, three, or four nitrogen atoms, one nitrogen and one sulfur atom, or one nitrogen and one oxygen atom. The term heteroaryl also includes bi-or tricyclic groups in which the aromatic heterocyclic ring is fused to one or two benzene rings. Representative heteroaryl groups are pyridyl, thienyl, indolyl, pyrazinyl, isoquinolyl, pyrrolyl, pyrimidyl, benzothienyl, furyl, benzo[b]furyl, imidazolyl, thiazolyl, carbazolyl, and the like. The term “heteroarylalkyl” denotes a heteroaryl group, as defined above, attached to the parent molecular moiety through an alkylene group. The term “heteroaryloxy” denotes a heteroaryl group, as defined above, attached to the parent molecular moiety through an oxygen atom. The term “heteroarylalkoxy” denotes a heteroarylalkyl group, as defined above, attached to the parent molecular moiety through an oxygen atom.

The expression C₁₋₆-alk (en/yn)yl means a C₁₋₆-alkyl, C₂₋₆-alkenyl or a C₂₋₆-alkynyl group. The expression C₃₋₈-cycloank(en)yl means a C₃₋₈-cycloalkyl- or cycloalkenyl group.

The term C₁₋₆-alkyl refers to a branched or unbranched alkyl group having from one to six carbon atoms inclusive, including but not limited to methyl, ethyl, 1-propyl, 2-propyl, 1-butyl, 2-butyl, 2-methyl-2-propyl and 2-methyl-1-propyl.

Similarly, C₂₋₆alkenyl and C₂₋₆alkynyl, respectively, designate such groups having from two to six carbon atoms, including one double bond and one triple bond respectively, including but not limited to ethenyl, propenyl, butenyl, ethynyl, propynyl and butynyl.

The term C₃₋₈-cycloalkyl designates a monocyclic or bicyclic carbocycle having three to eight C-atoms, including but not limited to cyclopropyl, cyclopentyl, cyclohexyl, etc.

The term C₃₋₈-cycloalkenyl designates a monocyclic or bicyclic carbocycle having three to eight C-atoms and including one double bond.

In the term C₃₋₈-cycloalkyl(en)yl-C₁₋₆-alk(en/yn)yl, C₃₋₈-cycloalk(en)yl and C₁₋₆-alk(en/yn)yl are as defined above.

The terms C₁₋₆-alk(en/yn)yloxy, C₁₋₆-alk(en/yn)yloxy-C₁₋₆-alk(en/yn)yl, C₁₋₆-alk(en/yn)ylsulfanyl, hydroxy-C₁₋₆-alk(en/yn)yl, halo-C₁₋₆-alk(en/yn)yl, halo-C₁₋₆-alk(en/yn)yloxy, C₁₋₆-alk(en/yn)ylsulfonyl, cyano-C₁₋₆-alk(en/yn)yl, hydroxy-C₁₋₆-alk en/yn)yl, NR^{x}R^{y}—C₁₋₆-alk(en/yn)yl, NR^{1′}CO—C₁₋₆ (en/yn) yl etc. designate such groups in which the C₁₋₆ (en/yn) yl is as defined above. The terms halo-, hydroxy-, cyano-etc. are to be understood as the C₁₋₆ (en/yn) yl-part can be substituted one or more times with such substituent. The term halo-designates halogen as defined above.

As used herein, the term C₁₋₆ (en/yn) yloxycarbonyl refers to groups of the formula —COO—C₁₋₆ (en/yn)yl, wherein C₁₋₆ (en/yn)yl are as defined above.

As used herein, the term acyl refers to formyl, C₁₋₆-alk(en/yn)ylcarbonyl, arylcarbonyl, aryl-C₁₋₆ alk(en/yn)ylcarbonyl, C₃₋₈-cycloalk(en)ylcarbonyl or a C₃₋₈-cycloalk(en)yl-C₁₋₆-alk(en/yn)yl-carbonyl group.

The term heterocycle designates rings such as 5-membered monocyclic rings such as 3H-1,2,3-oxathiazole, 1,3,2-oxathiazole, 1,2,3-dioxazole, 3H-1,2,3-dithiazole, 1,2,3-dithiazole, 1,2,3-oxadiazole, 1,2,3-thiadiazole, 1H-1,]2,3-triazole, isoxazole, oxazol, isothiazole, thiazole, 1H-imidazole, 1H-pyrazole, 1H-pyrrole, furan or thiophene and 6-membered monocyclic rings such as 1,2,3-oxathiazine, 1,2,4-oxathiazine, 1,2,5-oxathiazine, 1,4,2-oxathiazine, 1,4,3-oxathiazine, 1,2,3-dioxazine, 1,2,4-dioxazine, 4H-1,3,2-dioxazine, 1,4,2-dioxazine, 2H-1, 5,2-dioxazine, 1,2,3-dithiazine, 1,2,4-dithiazine, 4H-1,3,2-dithiazine, 1,4,2-dithiazine, 2H-1,5,2-dithiazine, 2H 1,2,3-oxadiazine, 2H-1,2,4-oxadiazine, 2H-1,2,5-oxadiazine, 2H-1,2,6-oxadiazine, 2H-1,3,4-oxadiazine, 2H-1,2,3-thia-diazine, 2H-1,2,4-thiadiazine, 2H-1,2,5-thiadiazine, 2H-1,2,6-thiadiazine, 2H-1,3,4-thiadiazine, 1,2,3-triazine, 1,2,4-triazine, 2H-1,2-oxazine, 2H-1,3-oxazine, 2H-1,4-oxazine, 2H-1,2-thiazine, 2H-1, 3-thiazine, 2H-1,4-thiazine, pyrazine, pyridazine, pyrimidine, 4H-1,3-oxathiin, 1,4-oxathiin, 4H-1,3-dioxin, 1,4-dioxin, 4H-1,3-dithiin, 1,4-dithiin, pyridine, 2H-pyran or 2H-thiin, bicyclic compounds wherein the above rings are fused to a benzene ring, such as indole, benzofuran, isobenzofuran, benzothiophen, benzimidazol, quinoline, isoquinoline, dihydroquinoline, or completely saturated rings such as morpholin, piperidin, azepin, piperazin, homopiperazin, and ring systems fused to a benzene ring, such as benzodioxan, benzodithiodioxan, benzo [[1,3] dioxol, dihydroindol, dihydrobenzofuran or dihydrobenzothiophen.

The term aryl refers to carbocyclic, aromatic systems such as phenyl, naphtyl, anthracene and phenantrene.

The terms aryloxy and aryl-C₁₋₆-alk(en/yn)yloxy refer to aryl as defined and C₁₋₆-alk(en/yn) yloxy as defined above.

The term carbocyclic refers to partly or completely saturated systems such as cyclohexen, indan or flurene.

The term heteroatom refers to atoms different from carbon and hydrogen, such as nitrogen, oxygen and sulphur.

Exemplary of organic acid addition salts according to the invention are those with maleic, fumaric, benzoic, ascorbic, succinic, oxalic, bis-methylenesalicylic, methanesulfonic, ethanedisulfonic, acetic, propionic, tartaric, salicylic, citric, gluconic, lactic, malic, mandelic, cinnamic, citraconic, aspartic, stearic, palmitic, itaconic, glycolic, p-aminobenzoic, glutamic, benzenesulfonic and theophylline acetic acids, as well as the 8-halotheophyllines, for example 8-bromotheophylline. Exemplary of inorganic acid addition salts according to the invention are those with hydrochloric, hydrobromic, sulfuric, sulfamic, phosphoric and nitric acids. The acid addition salts of the invention are preferably pharmaceutically acceptable salts formed with non-toxic acids.

Furthermore, the compounds used in the methods of this invention may exist in unsolvated as well as in solvated forms with pharmaceutically acceptable solvents such as water, ethanol and the like. In general, the solvated forms are considered equivalent to the unsolvated forms for the purposes of this invention.

Some of the compounds of the present invention contain chiral centres and such compounds exist in the form of isomers (e.g.. enantiomers). The invention includes all such isomers and any mixtures thereof including racemic mixtures.

Racemic forms can be resolved into the optical antipodes by known methods, for example, by chromatography on an optically active matrix. The compounds of the present invention may also be resolved by the formation of diastereomeric derivatives.

Methods standard in the art can be utilized for determining compounds that modulate the MLK family kinase protein activity, in particular MLK1. For example, MLK family kinase protein activity can be determined by measuring the activity of a substrate of the MLK family kinase. Such substrates are well known to those in the art. The substrate is preferably a member of the mitogen activated kinase family or substrates further down the pathway (e.g., JNK1, JNK2, JNK3, ERK1, ERK2, p38α, p38β, p38 γ, p38δ, MEK1, MEK2, MKK3, MKK4(SEK1), MEK5, MKK6, MKK7, jun AFT2 and ELK1, or other members of the pathway described in FIG. 1). Additionally, general substrates of Ser/Thr protein kinases such a myelin basic protein (MBP) can also be used. Reagents and methods for measuring the activity of the substrates are also known to those skilled in the art. The presence of MLK can also be determined by measuring the amount of MLK protein or mRNA encoding the MLK protein, such as the methods described below.

Assessment for At-Risk Haplotypes

A “haplotype,” as described herein, refers to a combination of genetic markers (“alleles”), such as those set forth in Tables 1 and 2. In a certain aspect, the haplotype can comprise one or more alleles, two or more alleles, three or more alleles, four or more alleles, or five or more alleles. The genetic markers are particular “alleles” at “polymorphic sites” associated with MAPK9. A nucleotide position at which more than one sequence is possible in a population (either a natural population or a synthetic population, e.g., a library of synthetic molecules) is referred to herein as a “polymorphic site”. Where a polymorphic site is a single nucleotide in length, the site is referred to as a single nucleotide polymorphism (“SNP”). For example, if at a particular chromosomal location, one member of a population has an adenine and another member of the population has a thymine at the same position, then this position is a polymorphic site, and, more specifically, the polymorphic site is a SNP. Polymorphic sites can allow for differences in sequences based on substitutions, insertions or deletions. Each version of the sequence with respect to the polymorphic site is referred to herein as an “allele” of the polymorphic site. Thus, in the previous example, the SNP allows for both an adenine allele and a thymine allele.

Typically, a reference sequence is referred to for a particular sequence. Alleles that differ from the reference are referred to as “variant” alleles. For example, the reference MAP3K9 sequence is described herein by SEQ ID NO: 1. The term, “variant MAP3K9”, as used herein, refers to a sequence that differs from SEQ ID NO: 1, but is otherwise substantially similar. The genetic markers that make up the haplotypes described herein are MAP3K9 variants.

Additional variants can include changes that affect a polypeptide, e.g., the MAP3K9 polypeptide. These sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by a MAP3K9 nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with a susceptibility to asthma can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of the polypeptide. The polypeptide encoded by the reference nucleotide sequence is the “reference” polypeptide with a particular reference amino acid sequence, and polypeptides encoded by variant alleles are referred to as “variant” polypeptides with variant amino acid sequences.

According to NCBI (National Center for Biotechnology Information), AceView, MAP3K9 is expressed at high levels. The sequence of this gene is supported by 48 sequences from 40 cDNA clones and produces, by alternative splicing, 5 different transcripts aDec03 (variant a), bDec03(variant b), cDec03 (variant c), dDec03, (variant d), and eDec03 (variant e), altogether encoding 5 different protein isoforms. As indicated in Table C.

TABLE C

| mRNA | |
|---|---|
| variant | NCBI Ace view site data |
| aDec03 | This complete mRNA is 7028 bp long. We annotate here the |
| 7028bp | sequence derived from the genome, although the best path through |
| AM_5541bp | the available clones differs from it in 1 position. It has 13 exons. It |
| | has a very long 3′ UTR. The premessenger covers 82.46 kb on the |
| | NCBI build 34, August 2003 genome. The protein (1071 aa, 118.9 kDa, |
| | pI 6.1) contains one SH3 domain motif, one protein kinase |
| | motif. It also contains a coiled coil stretch [Psort2]. Taxblast |
| | (threshold 10\hat{ }−3) tracks ancestors down to Viruses and Eukaryota. |
| bDec03 | This complete CDS mRNA is 5096 bp long. Its sequence exactly |
| AM_5096bp | matches the genome. It has 11 exons. It has a very long 3′ UTR. |
| | The premessenger covers 55.33 kb on the NCBI build 34, August |
| | 2003 genome. The protein (869 aa, 96.5 kDa, pI 6.9) contains one |
| | protein kinase motif. It also contains a coiled coil stretch [Psort2]. |
| | Taxblast (threshold 10\hat{ }−3) tracks ancestors down to Viruses and |
| | Eukaryota. |
| cDec03 | This mRNA is 1829 bp long. It. It has 3 exons. It may be |
| | incomplete at the 5′ end. The premessenger covers 9.56 kb on the |
| | NCBI build 34, August 2003 genome. The protein contains one |
| | SH3 domain motif, one protein kinase motif. Taxblast (threshold |
| | 10\hat{ }−3) tracks ancestors down to Viruses and Bacteria and |
| | Eukaryota. |
| dDec03 | This complete CDS mRNA is 572 bp long. It. It has a single exon. |
| 572bp | The premessenger covers 0.57 kb on the NCBI build 34, August |
| AM_569bp | 2003 genome. The protein (85 aa, 9.2 kDa, pI 7.7) contains no |
| | Pfam motif. It is predicted to localise in the cytoplasm [Psort2]. |
| eDec03 | This partial mRNA, 3′ incomplete is 411 bp long. It. It has a single |
| 411bp | exon. It is partial, truncated at the 3′ end. The premessenger covers |
| AM_411bp | 0.41 kb on the NCBI build 34, August 2003 genome. The partial |
| | protein (80 aa, 9.5 kDa, pI 5.3) contains no Pfam motif. It contains |
| | an ER membrane domain [Psort2]. Taxblast (threshold 10⁻³) tracks |
| | ancestors down to Bilateria. |

Haplotypes are a combination of genetic markers, e.g., particular alleles at polymorphic sites. The haplotypes described herein, e.g., having markers such as those shown in Table 1 are found more frequently in individuals with asthma than in individuals without asthma. Therefore, these haplotypes have predictive value for detecting a susceptibility to asthma in an individual. The haplotypes described herein are in some cases a combination of various genetic markers, e.g., SNPs and microsatellites. Therefore, detecting haplotypes can be accomplished by methods known in the art for detecting sequences at polymorphic sites, such as the methods described above.

In certain methods described herein, an individual who is at-risk for asthma is an individual in whom an at-risk haplotype is identified. In one aspect, the at-risk haplotype is one that confers a significant risk of asthma. In one aspect, significance associated with a haplotype is measured by an odds ratio. In a further aspect, the significance is measured by a percentage. In one aspect, a significant risk is measured as an odds ratio of at least about 1.2, including by not limited to: 1.2, 1.3, 1.4, 1.5, 1.6, 1.7, 1.8, and 1.9. In a further aspect, an odds ratio of at least 1.2 is significant. In a further aspect, an odds ratio of at least about 1.5 is significant. In a further aspect, a significant increase in risk is at least about 1.7 is significant. In a further aspect, a significant increase in risk is at least about 20%, including but not limited to about 25%, 30%, 35%, 40%, 45%, 50%, 55%, 60%, 65%, 70%, 75%, 80%, 85%, 90%, 95%, and 98%. In a further aspect, a significant increase in risk is at least about 50%. It is understood however, that identifying whether a risk is medically significant may also depend on a variety of factors, including the specific disease, the haplotype, and often, environmental factors.

An at-risk haplotype in, or comprising portions of, the MAP3K9 gene, in one where the haplotype is more frequently present in an individual at risk for asthma (affected), compared to the frequency of its presence in a healthy individual (control), and wherein the presence of the haplotype is indicative of susceptibility to asthma. As an example of a simple test for correlation would be a Fisher-exact test on a two by two table. Given a cohort of chromosomes the two by two table is constructed out of the number of chromosomes that include both of the haplotypes, one of the haplotype but not the other and neither of the haplotypes.

In certain aspects of the invention, at-risk haplotype is an at-risk haplotype within or near MAP3K9 that significantly correlates with a haplotype such as a halotype shown in Table 1. In other aspects, an at-risk haplotype comprises an at-risk haplotype within or near MAP3K9 that significantly correlates with susceptibility to asthma. In one aspect, the at-risk haplotype is characterized by the following microsatellite markers: DG14S1266 and DG14S205, wherein the presence of a 0,4 haplotype is diagnostic of asthma or a susceptibility to asthma. In another aspect, the at-risk haplotype is characterized by the following microsatellite markers: DG14S420 and DG14S399, wherein the presence of a 2,-l1 haplotype is diagnostic of asthma or a susceptibility to asthma. In another aspect, the at-risk haplotype is characterized by the following SNP markers: SG14S89, SG14S152, GS14S174 and SG14S184, wherein the presence of a 3,3,3,3 haplotype is diagnostic of asthma or a susceptibility to asthma. Other haplotype aspects are shown in Table 1.

Standard techniques for genotyping for the presence of SNPs and/or microsatellite markers can be used, such as fluorescent based techniques (Chen, et al., Genome Res. 9, 492 (1999)), PCR, LCR, Nested PCR and other techniques for nucleic acid amplification. In a preferred aspect, the method comprises assessing in an individual the presence or frequency of SNPs and/or microsatellites in, comprising portions of, the MAP3K9 gene, wherein an excess or higher frequency of the SNPs and/or microsatellites compared to a healthy control individual is indicative that the individual is susceptible to asthma. See, for example, Table 3 (below) and Table 5 for SNPs and markers that can form haplotypes that can be used as screening tools. These markers and SNPs can be identified in at-risk haploptypes. For example, an at-risk haplotype can include microsatellite markers and/or SNPs such as those set forth in Table 1. The presence of the haplotype is indicative of a susceptibility to asthma, and therefore is indicative of an individual who falls within a target population for the treatment methods described herein.

Haplotype analysis involves defining a candidate susceptibility locus using LOD scores. The defined regions are then ultra-fine mapped with microsatellite markers with an average spacing between markers of less than 100 Kb. All usable microsatellite markers that found in public databases and mapped within that region can be used. In addition, microsatellite markers identified within the deCODE genetics sequence assembly of the human genome can be used. The frequencies of haplotypes in the patient and the control groups using an expectation-maximization algorithm can be estimated (Dempster A. et al., 1977. J. R. Stat. Soc. B, 39:1-389). An implementation of this algorithm that can handle missing genotypes and uncertainty with the phase can be used. Under the null hypothesis, the patients and the controls are assumed to have identical frequencies. Using a likelihood approach, an alternative hypothesis where a candidate at-risk-haplotype, which can include the markers described herein, is allowed to have a higher frequency in patients than controls, while the ratios of the frequencies of other haplotypes are assumed to be the same in both groups is tested. Likelihoods are maximized separately under both hypotheses and a corresponding 1-df likelihood ratio statistic is used to evaluate the statistic significance.

To look for at-risk-haplotypes in the 1-lod drop, for example, association of all possible combinations of genotyped markers is studied, provided those markers span a practical region. The combined patient and control groups can be randomly divided into two sets, equal in size to the original group of patients and controls. The haplotype analysis is then repeated and the most significant p-value registered is determined. This randomization scheme can be repeated, for example, over 100 times to construct an empirical distribution of p-values. In a preferred aspect, a p-value of <0.05 is indicative of an at-risk haplotype.

A detailed discussion of haplotype analysis follows.

Haplotype Analysis

Our general approach to haplotype analysis involves using likelihood-based inference applied to NEsted MOdels. The method is implemented in our program NEMO, which allows for many polymorphic markers, SNPs and microsatellites. The method and software are specifically designed for case-control studies where the purpose is to identify haplotype groups that confer different risks. It is also a tool for studying LD structures.

When investigating haplotypes constructed from many markers, apart from looking at each haplotype individually, meaningful summaries often require putting haplotypes into groups. A particular partition of the haplotype space is a model that assumes haplotypes within a group have the same risk, while haplotypes in different groups can have different risks. Two models/partitions are nested when one, the alternative model, is a finer partition compared to the other, the null model, i.e, the alternative model allows some haplotypes assumed to have the same risk in the null model to have different risks. The models are nested in the classical sense that the null model is a special case of the alternative model. Hence traditional generalized likelihood ratio tests can be used to test the null model against the alternative model. Note that, with a multiplicative model, if haplotypes hᵢ and hⱼ are assumed to have the same risk, it corresponds to assuming that fᵢ/pᵢ=fⱼ/pⱼ where f and p denote haplotype frequencies in the affected population and the control population respectively.

One common way to handle uncertainty in phase and missing genotypes is a two-step method of first estimating haplotype counts and then treating the estimated counts as the exact counts, a method that can sometimes be problematic (e.g., see the information measure section below) and may require randomization to properly evaluate statistical significance. In NEMO, maximum likelihood estimates, likelihood ratios and p-values are calculated directly, with the aid of the EM algorithm, for the observed data treating it as a missing-data problem.

NEMO allows complete flexibility for partitions. For example, the first haplotype problem described in the Methods section on Statistical analysis considers testing whether h₁ has the same risk as the other haplotypes h₂, . . . , hₖ. Here the alternative grouping is [h₁], [h₂, . . . , hₖ] and the null grouping is [h₁, . . . , hₖ]. The second haplotype problem in the same section involves three haplotypes h₁=G0, h₂=GX and h₃+AX, and the focus is on comparing h₁ and h₂. The alternative grouping is [h₁], [h₂], [h₃] and the null grouping is [h₁, h₂], [h₃]. If composite alleles exist, one could collapse these alleles into one at the data processing stage, and performed the test as described. This is a perfectly valid approach, and indeed, whether we collapse or not makes no difference if there were no missing information regarding phase. But, with the actual data, if each of the alleles making up a composite correlates differently with the SNP alleles, this will provide some partial information on phase. Collapsing at the data processing stage will unnecessarily increase the amount of missing information. A nested-models/partition framework can be used in this scenario. Let h₂ be split into h₂ₐ, h_{2b}, h₂ₑ, and h₃ be split into h₃ₐ, h_{3b}, . . . , h₃ₑ. Then the alternative grouping is [h₁], [h₂ₐ, h_{2b}, . . . , h₂ₑ], [h₃ₐ, h_{3b}, . . , h₃ₑ] and the null grouping is [h₁, h₂ₐ, h_{2b}, . . . , h₂ₑ], [h₃ₐ, h_{3b}, . . . , h₃ₑ]. The same method can be used to handle composite where collapsing at the data processing stage is not even an option since L_{C} represents multiple haplotypes constructed from multiple SNPs. Alternatively, a 3-way test with the alternative grouping of [h₁], [h₂ₐ, h_{2b}, . . . , h₂ₑ], [h₃ₐ, h_{3b}, . . . , h₃ₑ] versus the null grouping of [h₁, h₂ₐ, h_{2b}, . . . , h₂ₑ, h₃ₐ, h_{3b}, . . . , h₃ₑ] could also be performed. Note that the generalized likelihood ratio test-statistic would have two degrees of freedom instead of one.

Measuring Information

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. Interestingly, one can measure information loss by considering a two-step procedure to evaluating statistical significance that appears natural but happens to be systematically anti-conservative. Suppose we calculate the maximum likelihood estimates for the population haplotype frequencies calculated under the alternative hypothesis that there are differences between the affected population and control population, and use these frequency estimates as estimates of the observed frequencies of haplotype counts in the affected sample and in the control sample. Suppose we then perform a likelihood ratio test treating these estimated haplotype counts as though they are the actual counts. We could also perform a Fisher's exact test, but we would then need to round off these estimated counts since they are in general non-integers. This test will in general be anti-conservative because treating the estimated counts as if they were exact counts ignores the uncertainty with the counts, overestimates the effective sample size and underestimates the sampling variation. It means that the chi-square likelihood-ratio test statistic calculated this way, denoted by Λ*, will in general be bigger than Λ, the likelihood-ratio test-statistic calculated directly from the observed data as described in methods. But Λ* is useful because the ratio Λ/Λ* happens to be a good measure of information, or 1−(Λ/Λ*) is a measure of the fraction of information lost due to missing information. This information measure for haplotype analysis is described in Nicolae and Kong, Technical Report 537, Department of Statistics, University of Statistics, University of Chicago, Revised for Biometrics (2003) as a natural extension of information measures defined for linkage analysis, and is implemented in NEMO.

Statistical Analysis.

For single marker association to the disease, the Fisher exact test can be used to calculate two-sided p-values for each individual allele. All p-values are presented unadjusted for multiple comparisons unless specifically indicated. The presented frequencies (for microsatellites, SNPs and haplotypes) are allelic frequencies as opposed to carrier frequencies. To minimize any bias due the relatedness of the patients who were recruited as families for the linkage analysis, first and second-degree relatives can be eliminated from the patient list. Furthermore, the test can be repeated for association correcting for any remaining relatedness among the patients, by extending a variance adjustment procedure described in Risch, N. & Teng, J. (Genome Res., 8:1278-1288 (1998)). The relative power of family-based and case-control designs for linkage disequilibrium studies of complex human diseases I. DNA pooling. (ibid) for sibships so that it can be applied to general familial relationships, and present both adjusted and unadjusted p-values for comparison. The differences are in general very small as expected. To assess the significance of single-marker association corrected for multiple testing we carried out a randomisation test using the same genotype data. Cohorts of patients and controls can be randomized and the association analysis redone multiple times (e.g., up to 500,000 times) and the p-value is the fraction of replications that produced a p-value for some marker allele that is lower than or equal to the p-value we observed using the original patient and control cohorts.

For both single-marker and haplotype analyses, relative risk (RR) and the population attributable risk (PAR) can be calculated assuming a multiplicative model (haplotype relative risk model), (Terwilliger, J. D. & Ott, J., Hum Hered, 42, 337-46 (1992) and Falk, C. T. & Rubinstein, P, Ann Hum Genet 51 (Pt 3), 227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes hᵢ and hⱼ, risk(hᵢ)/risk(hⱼ)=(fᵢ/pᵢ)/(fⱼ/pⱼ), where f and p denote respectively frequencies in the affected population and in the control population. While there is some power loss if the true model is not multiplicative, the loss tends to be mild except for extreme cases. Most importantly, p-values are always valid since they are computed with respect to null hypothesis.

In general, haplotype frequencies are estimated by maximum likelihood and tests of differences between cases and controls are performed using a generalized likelihood ratio test (Rice, J. A. Mathematical Statistics and Data Analysis, 602 (International Thomson Publishing, (1995)). deCODE's haplotype analysis program called NEMO, which stands for NEsted MOdels, can be used to calculate all the haplotype results. To handle uncertainties with phase and missing genotypes, it is emphasized that we do not use a common two-step approach to association tests, where haplotype counts are first estimated, possibly with the use of the EM algorithm, Dempster, (A. P., Laird, N. M. & Rubin, D. B., Journal of the Royal Statistical Society B. 39, 1-38 (1971)) and then tests are performed treating the estimated counts as though they are true counts, a method that can sometimes be problematic and may require randomisation to properly evaluate statistical significance. Instead, with NEMO, maximum likelihood estimates, likelihood ratios and p-values are computed with the aid of the EM-algorithm directly for the observed data, and hence the loss of information due to uncertainty with phase and missing genotypes is automatically captured by the likelihood ratios. Even so, it is of interest to know how much information is retained, or lost, due to incomplete information. Described herein is such a measure that is natural under the likelihood framework. For a fixed set of markers, the simplest tests performed compare one selected haplotype against all the others. Call the selected haplotype hᵢ and the others h₂, . . . , hₖ. Let p₁, . . . , pₖ denote the population frequencies of the haplotypes in the controls, and f₁, . . . , fₖ denote the population frequencies of the haplotypes in the affecteds. Under the null hypothesis, fᵢ=pᵢ for all i. The alternative model we use for the test assumes h₂, . . . , hₖ to have the same risk while h₁ is allowed to have a different risk. This implies that while pi can be different from f₁, fᵢ/(f₂+ . . . +fₖ)=pᵢ/(p₂+ . . . +pₖ)=βᵢ for i=2, . . . , k. Denoting f₁/p₁ by r, and noting that β₂+ . . . +βₖ=1, the test statistic based on generalized likelihood ratios is
Λ=2[l(\hat{r}, \hat{p}₁, \hat{β}₂, . . . , \hat{β}ₖ₋₁)−l(1, \tilde{p}₁, \tilde{β}₂, . . . , \tilde{β}ₖ₋₁)]
where l denotes logₑlikelihood and ~and ˆdenote maximum likelihood estimates under the null hypothesis and alternative hypothesis respectively. A has asymptotically a chi-square distribution with 1-df, under the null hypothesis. Slightly more complicated null and alternative hypotheses can also be used. For example, let h₁ be G0, h₂ be GX and h₃ be AX. When comparing G0 against GX, i.e., this is the test which gives estimated RR of 1.46 and p-value=0.0002, the null assumes G0 and GX have the same risk but AX is allowed to have a different risk. The alternative hypothesis allows, for example, three haplotype groups to have different risks. This implies that, under the null hypothesis, there is a constraint that f₁/p₁=f₂/p₂, or w=[f₁/p₁]/[f₂/p₂]=1. The test statistic based on generalized likelihood ratios is
Λ=2[l(\hat{p}₁, \hat{f}₁, \hat{p}₂, ŵ)−l(\tilde{p}₁, \tilde{f}₁, \tilde{p}₂, 1)]
that again has asymptotically a chi-square distribution with 1-df under the null hypothesis. If there are composite haplotypes (for example, h₂ and h₃), that is handled in a natural manner under the nested models framework.

LD between pairs of SNPs can be calculated using the standard definition of D′ and R² (Lewontin, R., Genetics 49, 49-67 (1964) and Hill, W. G. & Robertson, A. Theor. Appl. Genet. 22, 226-231 (1968)). Using NEMO, frequencies of the two marker allele combinations are estimated by maximum likelihood and deviation from linkage equilibrium is evaluated by a likelihood ratio test. The definitions of D′ and R² are extended to include microsatellites by averaging over the values for all possible allele combination of the two markers weighted by the marginal allele probabilities. When plotting all marker combination to elucidate the LD structure in a particular region, we plot D′ in the upper left corner and the p-value in the lower right corner. In the LD plots the markers can be plotted equidistant rather than according to their physical location, if desired.

Statistical Methods for Linkage Analysis

Multipoint, affected-only allele-sharing methods can be used in the analyses to assess evidence for linkage. Results, both the LOD-score and the non-parametric linkage (NPL) score, can be obtained using the program Allegro (Gudbjartsson et al., Nat. Genet. 25:12-3, 2000). Our baseline linkage analysis uses the Spairs scoring function (Whittemore, A. S., Halpern, J. (1994), Biometrics 50:118-27; Kruglyak L, et al. (1996), Am J Hum Genet 58:1347-63), the exponential allele-sharing model (Kong, A. and Cox, N. J. (1997), Am J Hum Genet 61:1179-88) and a family weighting scheme that is halfway, on the log-scale, between weighting each affected pair equally and weighting each family equally. The information measure we use is part of the Allegro program output and the information value equals zero if the marker genotypes are completely uninformative and equals one if the genotypes determine the exact amount of allele sharing by decent among the affected relatives (Gretarsdottir et al., Am. J. Hom. Genet, 70:593-603, (2002)). We computed the P-values two different ways and here report the less significant result. The first P-value can be computed on the basis of large sample theory; the distribution of Zₗᵣ=√(2[logₑ(10)LOD]) approximates a standard normal variable under the null hypothesis of no linkage (Kong, A. and Cox, N. J. (1997), Am J Hum Genet 61:1179-88). The second P-value can be calculated by comparing the observed LOD-score with its complete data sampling distribution under the null hypothesis (e.g., Gudbjartsson et al., Nat. Genet. 25:12-3, 2000). When the data consist of more than a few families, these two P-values tend to be very similar.

Methods for Deriving mRNA Expression Data:

Real Time (RT)-PCR is used to examine RNA levels of MLK-1 in blood cells and lung tissue of asthma patients and controls. Total RNA is extracted using Trizol and purified with Qia RNaeasy spin columns (Qiagen Inc. Valencia, Calif.). Two μg of total RNA is treated with DNaseI and the RNA was reverse transcribed using the TaqMan Reverse Transcription Reagents kit (N808-0234) and random hexamers. Five ABI SYBR green assays are constructed for estimation of MLK-1 transcripts (variant A-E, Table A). PCR reactions are carried out on a 384 well plate in a total volume of 10 μl on the Applied Biosystems PRISM 7900HT Sequence Detection System (95° C. for 10 minutes followed by 40 cycles of 95° C. for 15 seconds, 60° C. for 1 minute; with a subsequent dissociation step; 95° C. for 15 seconds, 60° C. for 15 seconds, 95° C. for 15 seconds which identifies melting temperatures of PCR products, thus assuring it specificity). The reaction consisted of 1 μl of cDNA, 1× SYBR Green PCR Master Mix (part number 4309155) and 900 nM primers. All reactions were run in quadruplicates for both the five MLK-1 isoforms and the housekeeping gene (Beta actin). The RNA levels (copy numbers) are determined using sequence specific probes that hybridize to PCR products of MLK kinases (e.g., MLK1) by employing the 5′→3′ exonuclease activity of Taq DNA polymerase on RNA samples that are isolated from cells that have been exposed to specific cytokine activators that activate the JNK pathway (such as IL1b and TNFα) vs vehicle alone (i.e., no activation). The TaqMan probe consists of a site-specific sequence labeled with a fluorescent reporter dye and a fluorescent quencher dye. During PCR the TaqMan probe hybridizes to its complementary single strand DNA sequence within the PCR target. When amplification occurs the TaqMan probe is degraded due to the 5′→3′ exonuclease activity of Taq DNA polymerase, thereby separating the quencher from the reporter during extension. Due to the release of the quenching effect on the reporter, the fluorescence intensity of the reporter dye increases. During the entire amplification process this light emission increases exponentially, the final level being measured by spectrophotometry after termination of the PCR. In addition to the MLK kinase (e.g., MLK1) sequence-specific TaqMan probes, SYBR Green are also used as a fluorescent dye. This dye fluoresces only when bound to double-stranded DNA, i.e., when MLK kinase (e.g., MLK1) unique primers bind and successfully allow for Taq DNA polymerase extension of DNA fragment representing the MLK kinase (e.g., MLK1) gene. The use of primers located in unique exons will ensure that Taq DNA polymerase DNA fragments represent the mature RNA structure, furthermore the use of MLK kinase (e.g., MLK1) sequence-specific TaqMan probes allows for discrimation between different RNA splice variants. Three calibrators are used to correct the quantity of the repeated samples for plate-to-plate variation. All values are subsequently normalized to standard corrected housekeeping gene values.

Assessment of MAP3K9 Gene Dysregulation

In one aspect, the invention relates to methods of measuring RNA levels of the MLK kinases (e.g., MLK1) using Real-Time Quantitative PCR assay in which oligonucleotides specific for members of the MLK kinase family (e.g., MLK1) are used to amplify reverse transcribed RNA (c-DNA) on RNA samples that are isolated from blood leukocytes or other tissue samples. The method includes obtaining a sample of cells from the patient, and determining RNA levels using sequence specific probes that hybridize to PCR products of MLK kinases (e.g., MLK1) by employing the 5′→3′ exonuclease activity of Taq DNA polymerase on RNA samples that are isolated from cells that have been exposed to specific cytokine activators that activate the JNK pathway (such as IL1b or TNFα) vs vehicle alone (i.e., no activation). In one aspect, the TaqMan probe consists of a site-specific sequence labeled with a fluorescent reporter dye and a fluorescent quencher dye wherein, during the PCR reaction, the TaqMan probe hybridizes to its complementary single strand DNA sequence within the PCR target, the final level being measured by spectrophotometry after termination of the PCR. In another aspect, MLK kinase expression (e.g., MLK1) is determined using SYBR Green as a fluorescent dye. This dye fluoresces only when bound to double-stranded DNA, i.e. when MLK kinase (e.g., MLK1) uniquely designed primers bind and allow for successesful Taq DNA polymerase extension of DNA fragment representing the MLK kinase (e.g., MLK1) gene. As such, the use of MLK kinase (e.g., MLK1) sequence-specific TaqMan probes allows for discrimation between different RNA splice variants. In yet another aspect, the invention is directed at methods that determine the role of MAP3k9 or its pathway-related genes, by obtaining a sample of cells from patients with asthma or other respiratory or inflammatory disorders, determining RNA levels of MAP3k9 or its pathway related genes in cells exposed to pathway specific activators (such as IL1b or TNFα) or vehicle alone (no activation), and comparing them with reference RNA levels of the gene in cells isolated from subjects without asthma or other inflammatory/respiratory disorders. In another aspect, the invention relates to methods for predicting efficacy of an inhibitor drug, including obtaining a sample of cells from patients with asthma or another respiratory/inflammatory disorder, determining RNA levels of MAP3k9 or its pathway related genes in cells isolated from patients who are taking the drug compared to those who are not taking the drug. In another aspect, the invention relates to methods for predicting efficacy of an inhibitor drug, including obtaining a sample of cells from patients with asthma or other respiratory/inflammatory disorders, determining RNA levels of MAP3k9 or its pathway related genes after exposure of the cells to the inhibitor drug in vitro.

Monitoring Progress of Treatment

The current invention also pertains to methods of monitoring the response of an individual, such as an individual in one of the target populations described above, to treatment with a MLK family kinase inhibitor.

Because the level of inflammatory markers can be elevated in individuals who are in the target populations described above, an assessment of the level of inflammatory markers of the individual both before, and during, treatment with the MLK family kinase inhibitor may indicate whether the treatment has successfully decreased production of MLKs in the airway wall (such as in ASM cells) or in bone-marrow derived inflammatory cells (such as peripheral blood mononuclear (PBM cells). For example, in one aspect of the invention, an individual who is a member of a target population as described above (e.g., an individual at risk for asthma, such as an individual who is at risk due to a MAP3K9 haplotype) can be assessed for response to treatment with a MLK family kinase inhibitor, by examining the individuals MLK kinase levels in different cells and body fluids. Blood, serum, plasma or urinary MLKs kinases (e.g., MLK1), or ex vivo production of MLK kinases (e.g., MLK1), can be measured before, and during or after treatment with the MLK family kinase inhibitor. The MLK or MLK family kinase level before treatment is compared with the MLK family kinase level during or after treatment. The efficacy of treatment is indicated by a decrease in MLK production: a level of MLK family kinase during or after treatment that is significantly lower than the level of MLK family kinase before treatment, is indicative of efficacy. A level that is lower during or after treatment can be shown, for example, by decreased serum or urinary MLKs, or decreased ex vivo production of MLK family kinases. A level that is “significantly lower”, as used herein, is a level that is less than the amount that is typically found in control individual(s), or is less in a comparison of disease risk in a population associated with the other bands of measurement (e.g., the mean or median, the highest quartile or the highest quintile) compared to lower bands of measurement (e.g., the mean or median, the other quartiles; the other quintiles).

For example, in one aspect of the invention, the level of a MLK family kinase is assessed in an individual before treatment with a MLK family kinase inhibitor; and during or after treatment with the MLK family kinase inhibitor, and the levels are compared. A level of the MLK family kinase during or after treatment that is significantly lower than the level of the MLK family kinase before treatment, is indicative of efficacy of treatment with the MLK family kinase inhibitor. In another aspect, production of a MLK family kinase is analyzed in a first test sample from the individual, and is also determined in a second test sample from the individual, during or after treatment with the MLK family kinase inhibitor, and the level of production in the first test sample is compared with the level of production of the MLK family kinase in the second test sample. A level of the MLK family kinase in the second test sample that is significantly lower than the level of the MLK family kinase in the first test sample is indicative of efficacy of treatment with the MLK family kinase inhibitor.

In another aspect of the invention, an individual who is a member of a target population of individuals at risk for asthma (e.g., an individual in a target population described above, can be assessed for response to treatment with a MLK family kinase inhibitor, by examining levels of inflammatory markers in the individual. For example, levels of an inflammatory marker in an appropriate test sample (e.g., serum, plasma or urine) can be measured before, and during or after treatment with the MLK family kinase inhibitor. The level of the inflammatory marker before treatment is compared with the level of the inflammatory marker during or after treatment. The efficacy of treatment is indicated by a decrease in the level of the inflammatory marker, that is, a level of the inflammatory marker during or after treatment that is significantly different (e.g., significantly lower), than the level of inflammatory marker before treatment, is indicative of efficacy. Representative inflammatory markers include plasma IL-2, IL-6, IL-1β and TNF-α levels and exhaled nitric oxide (NO).

Pharmaceutical Compositions

The present invention also pertains to pharmaceutical compositions comprising agents described herein, for example, an agent that is a MLK family kinase inhibitor as described herein. For instance, a MLK family kinase inhibitor can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc. Nebulized formulation for inhalation can include sodium chloride, sodium saccharine or sorbitani trioleas, whereas inhalation via compressed carbonated formulation in a puffer can include 1,1,1,2-tetrafluoroethanum, monofluorotrichloromethanum tetrafluorodichloroaethanum or diflurodichloromethanum.

Methods of introduction of these compositions include, but are not limited to, intradermal, intramuscular, intraperitoneal, intraocular, intravenous, subcutaneous, topical, oral, inhaled and intranasal. Other suitable methods of introduction can also include gene therapy (as described below), rechargeable or biodegradable devices, particle acceleration devices (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration. Administration by inhalation includes a mixture of the active drug and the above mentioned ingredients.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium,. ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms, and should be decided according to the judgment of a practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together-in any combination, present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

Nucleic Acids of the Invention

MAP3K9 Nucleic Acids, Portions and Variants

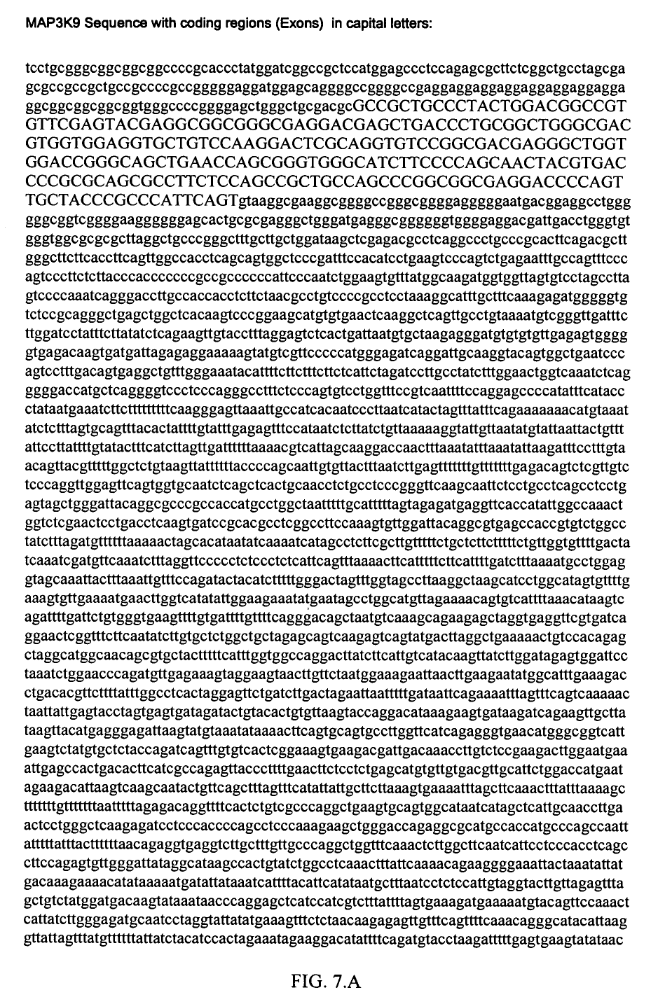

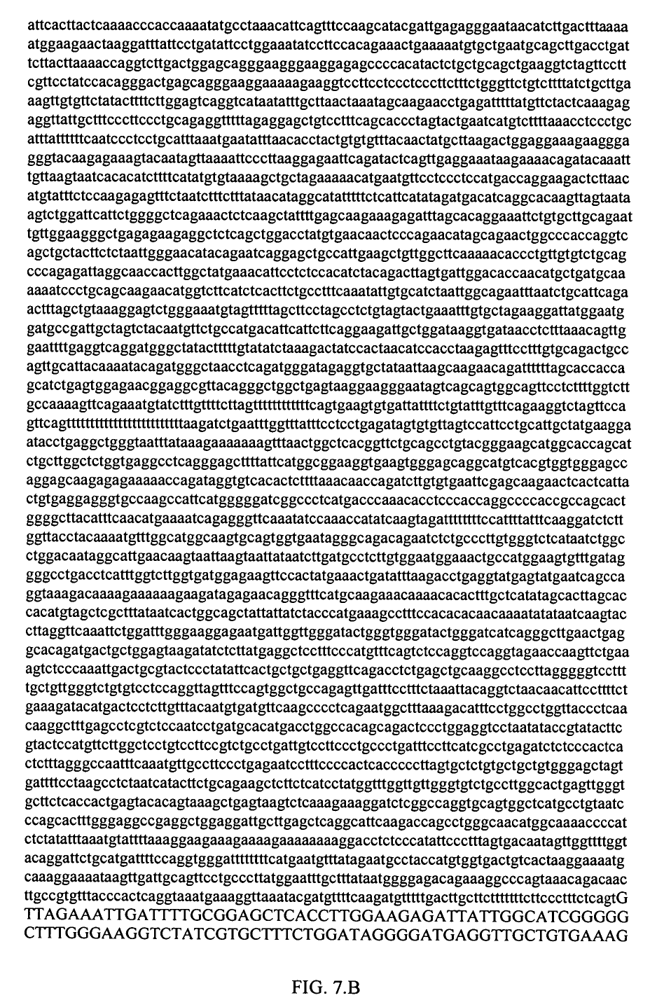

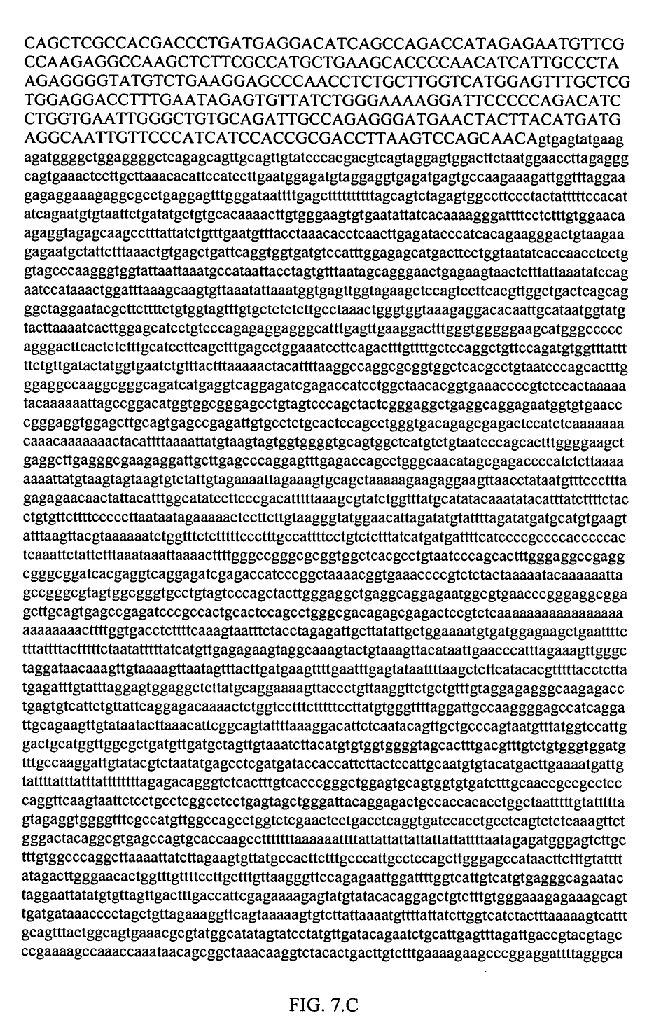

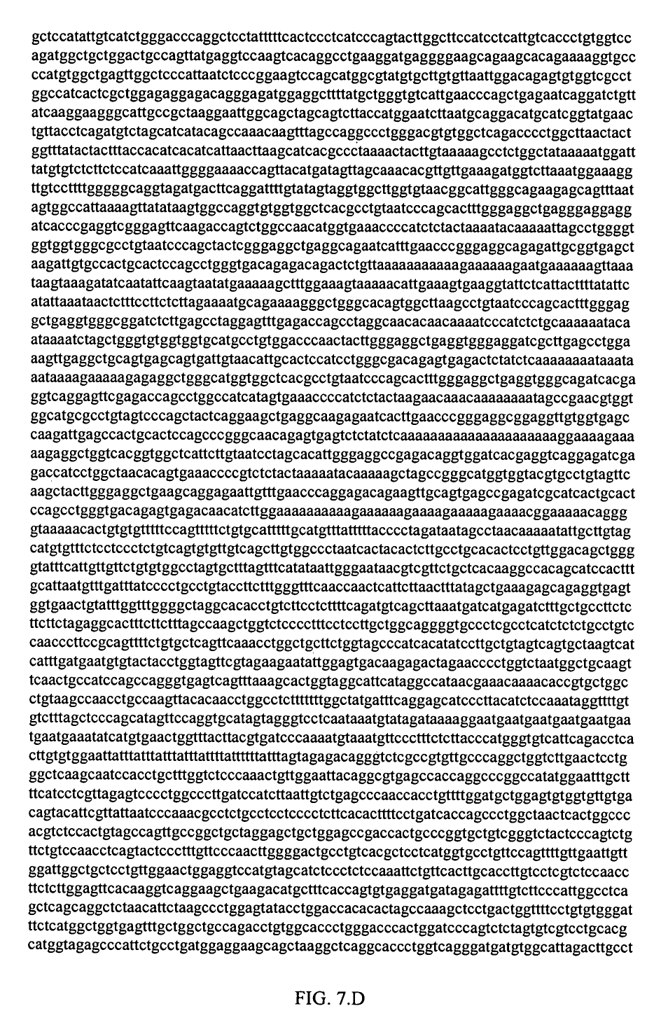

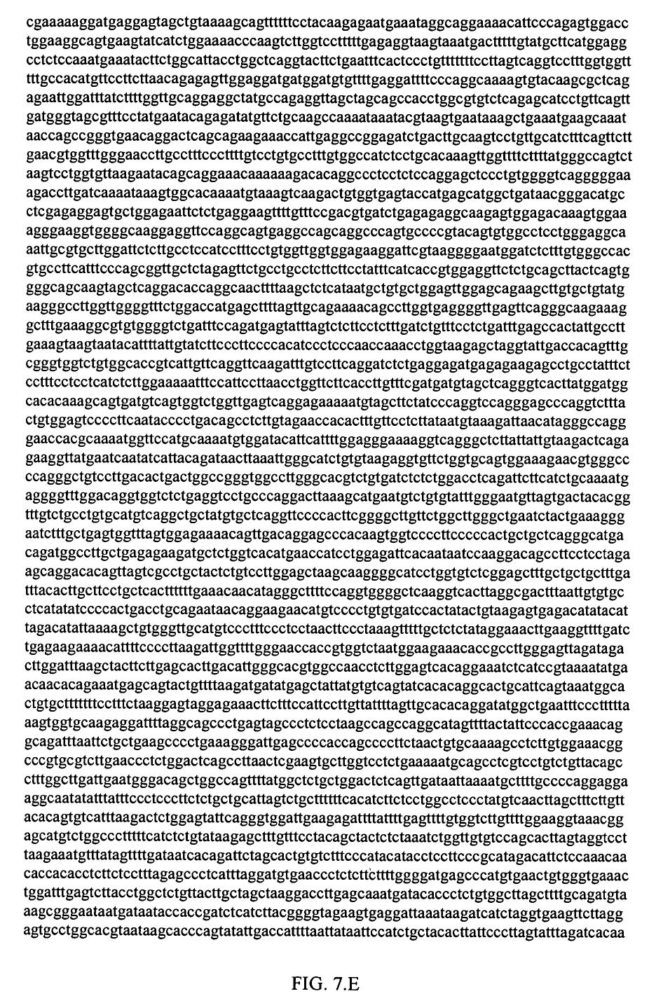

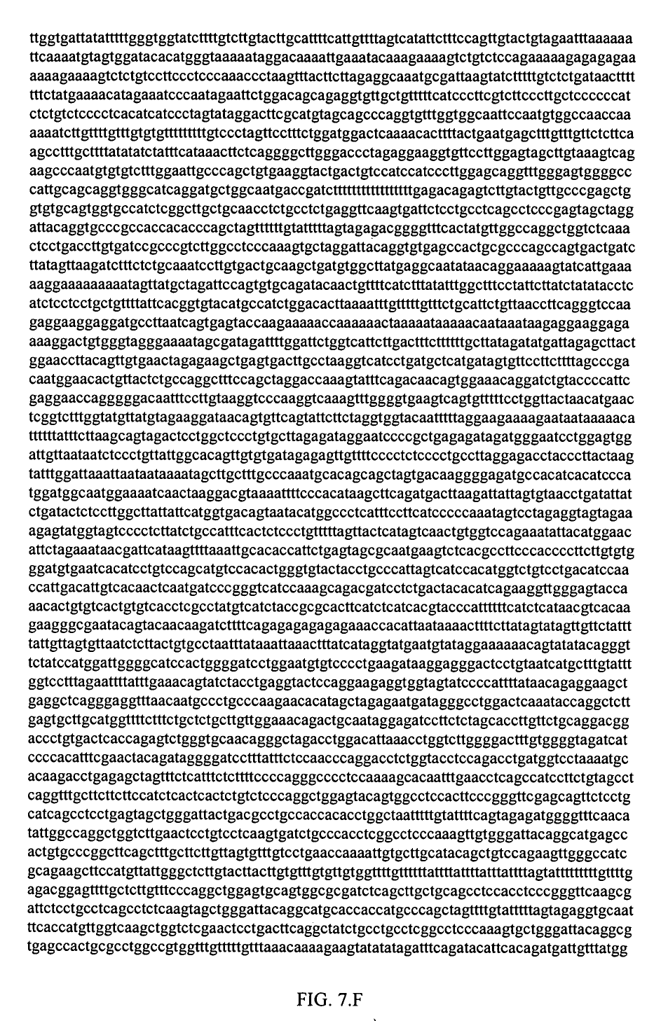

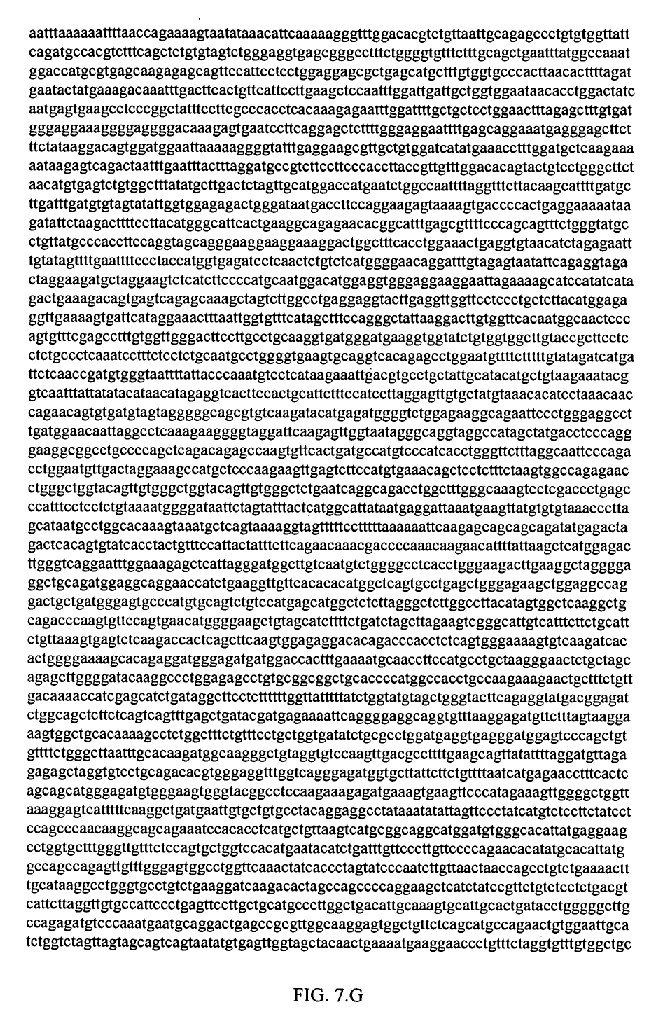

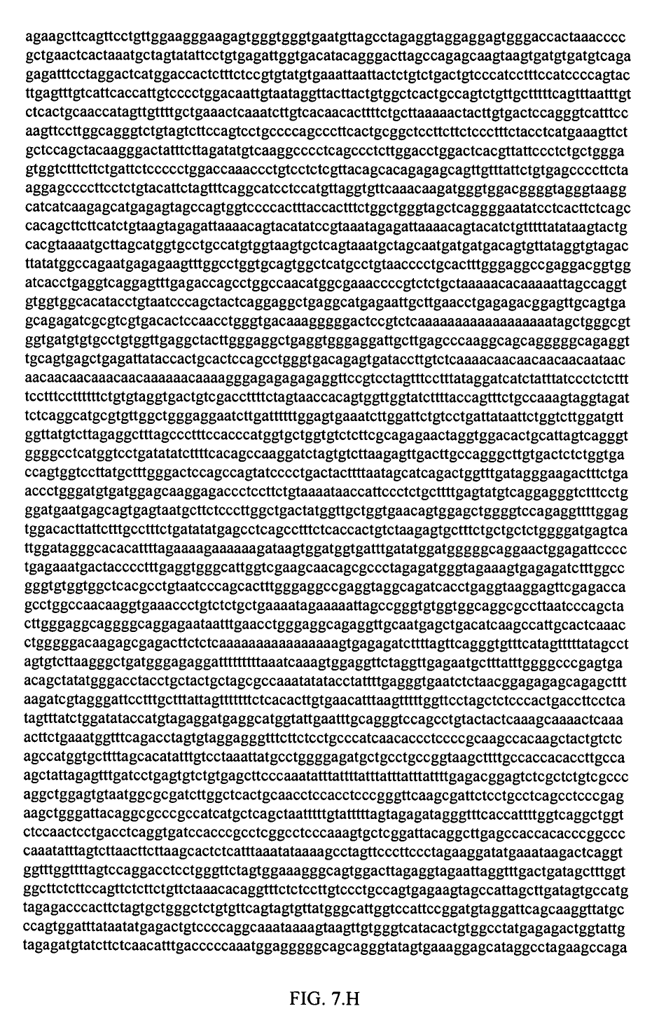

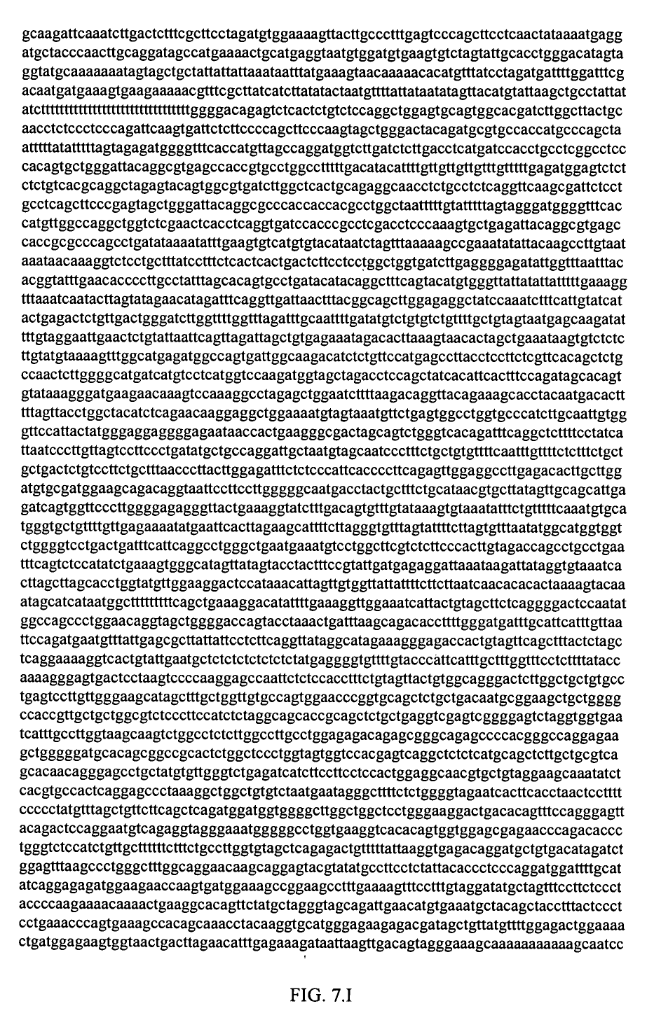

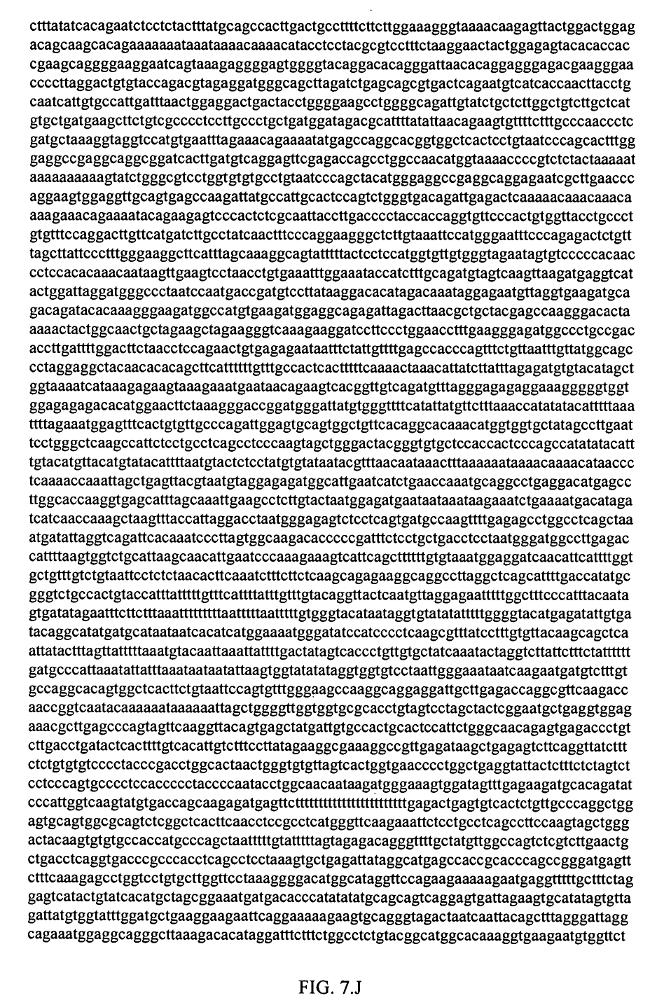

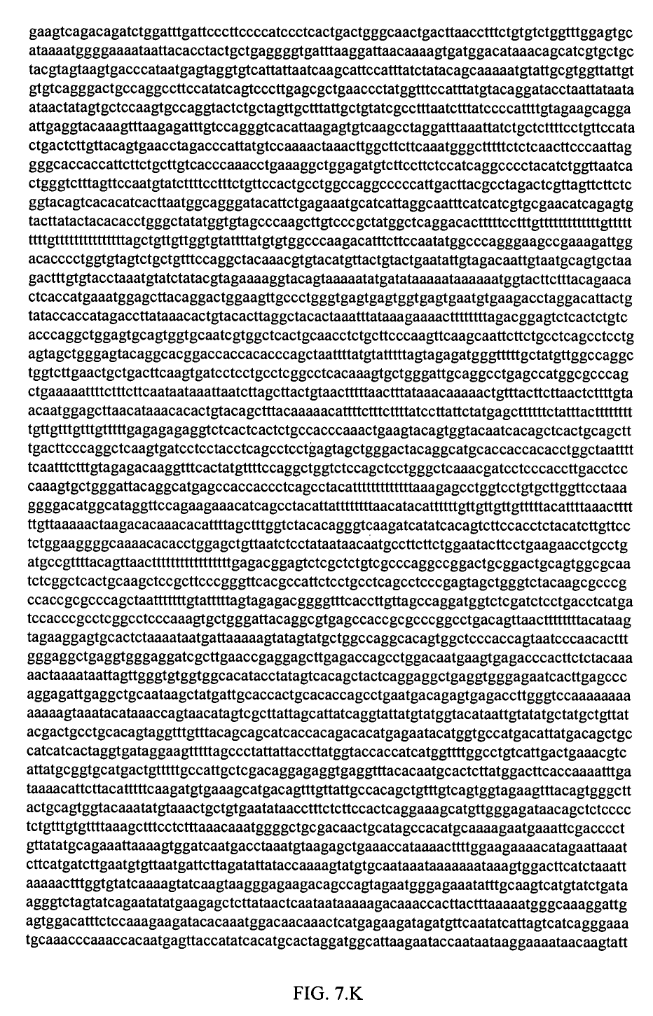

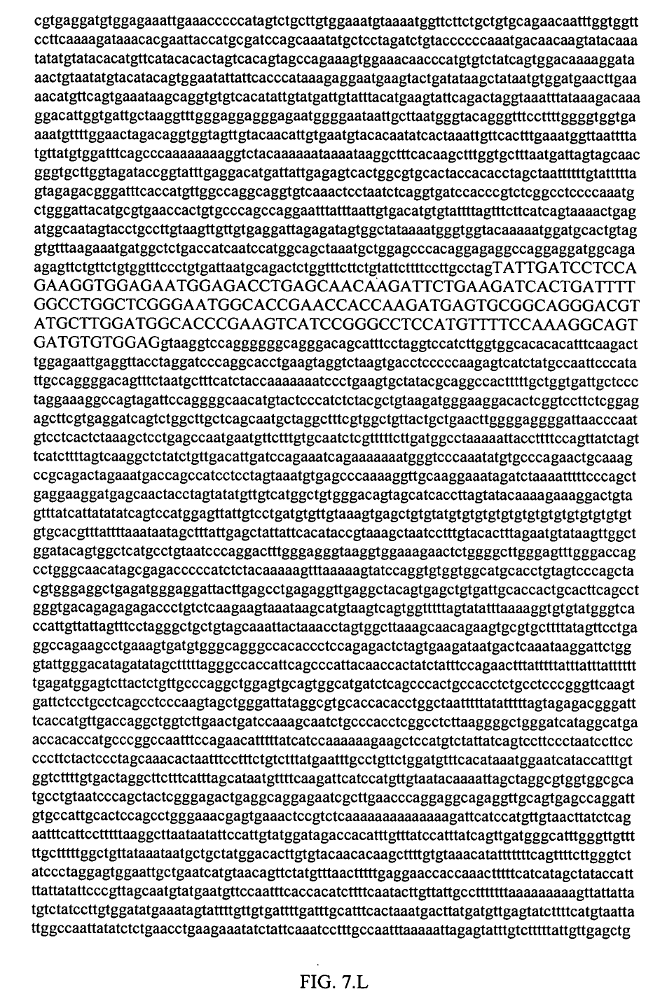

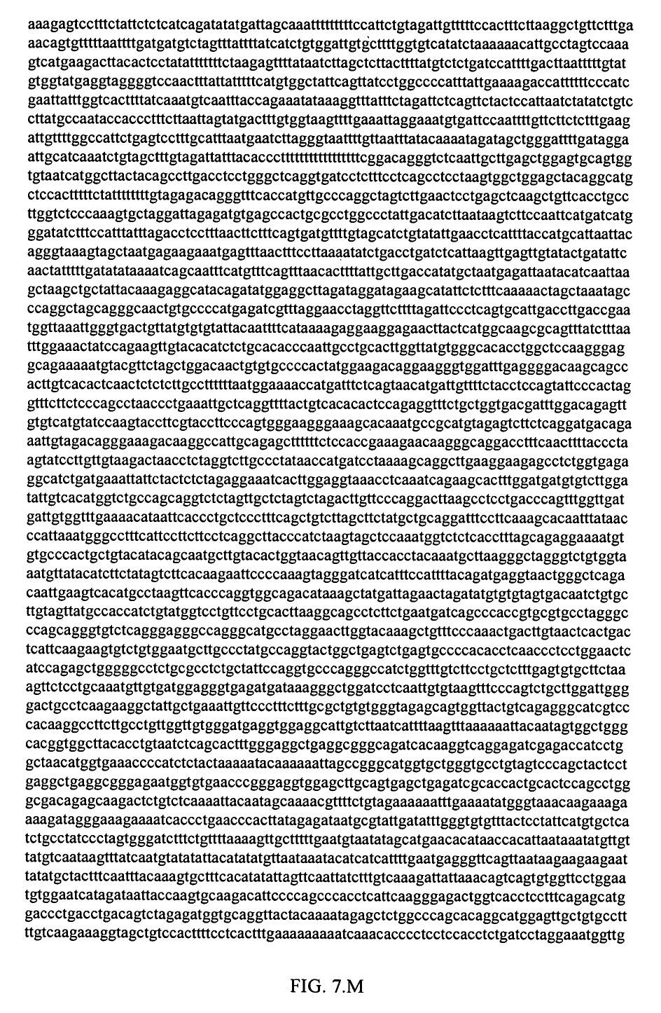

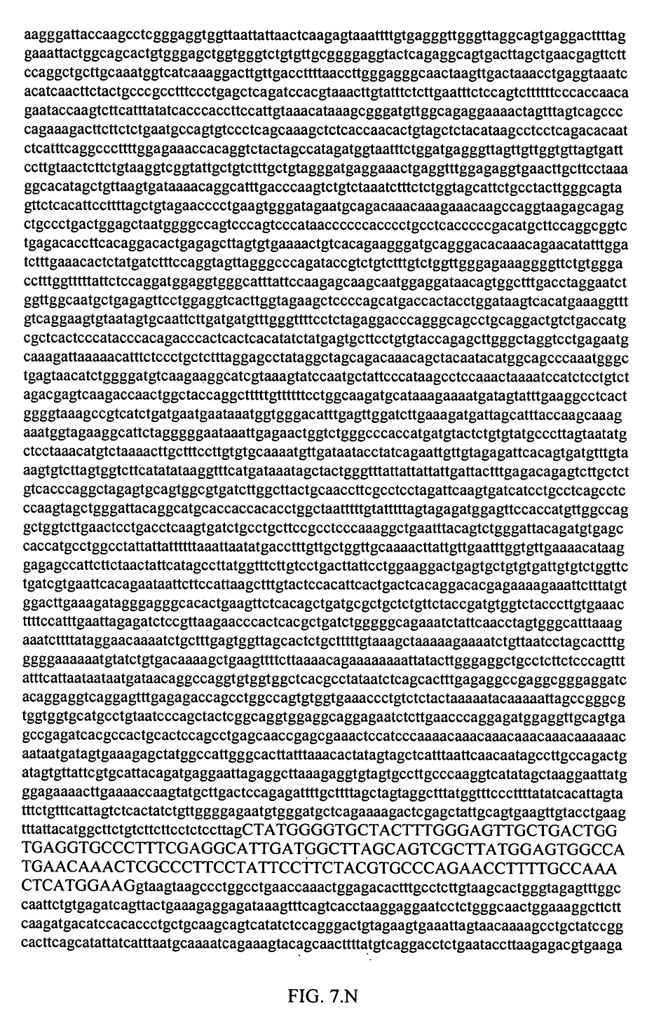

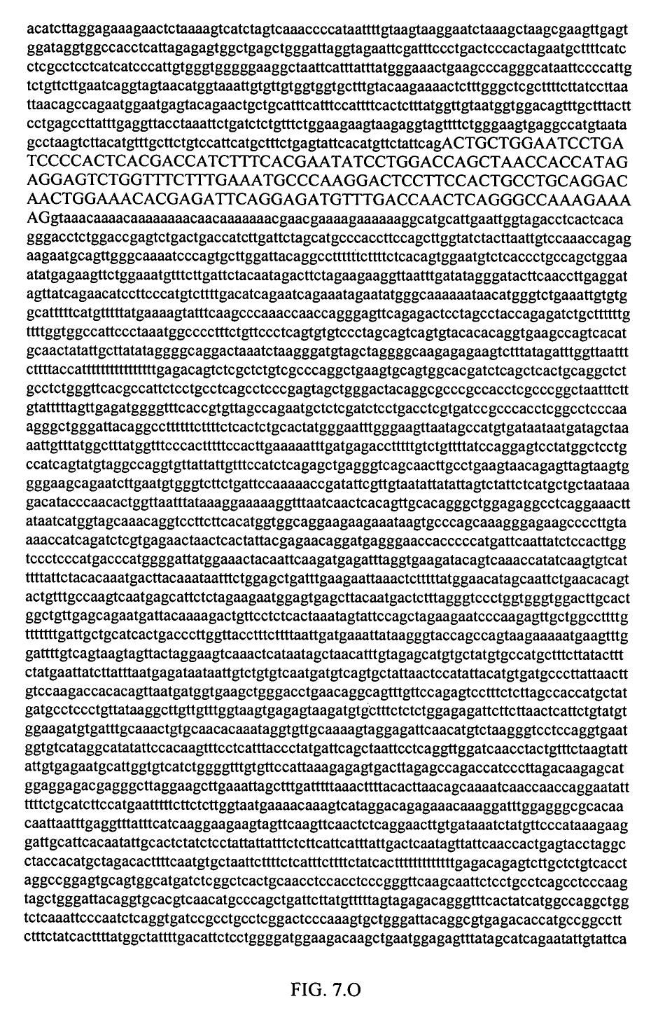

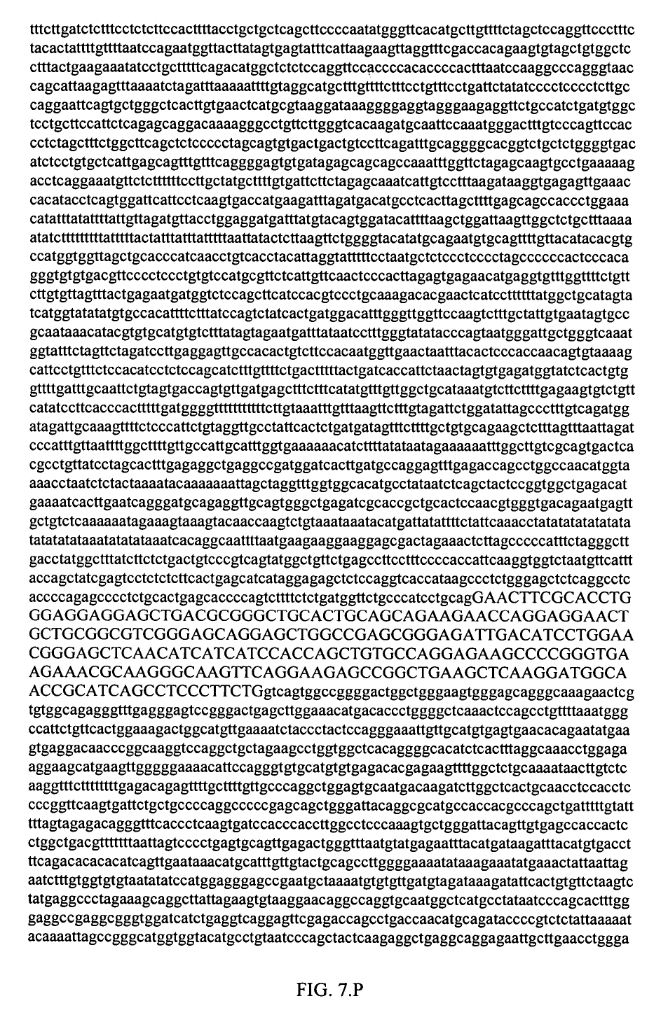

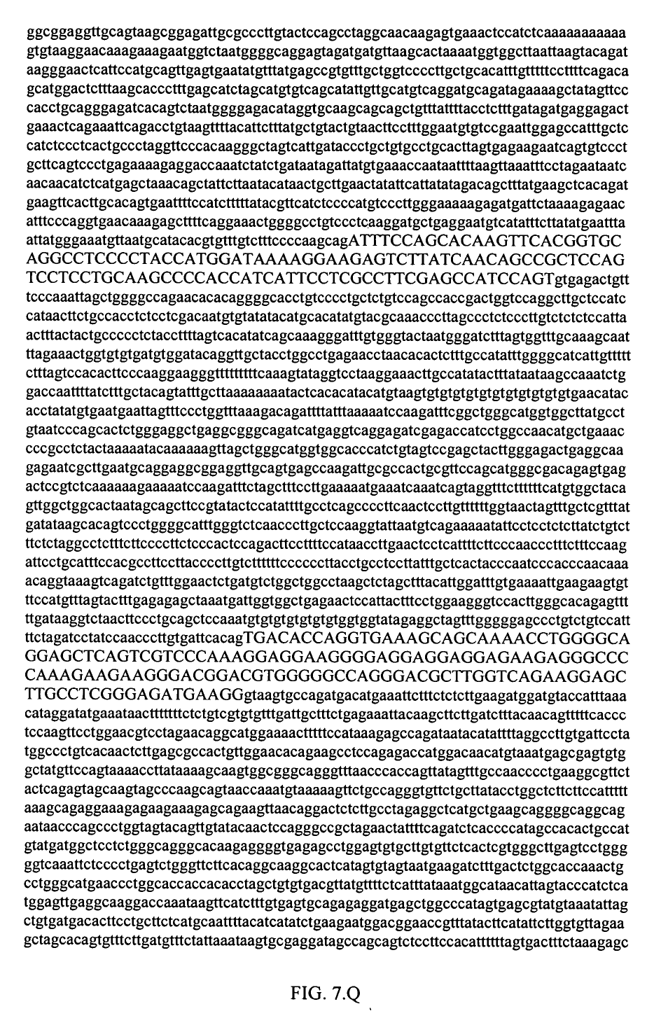

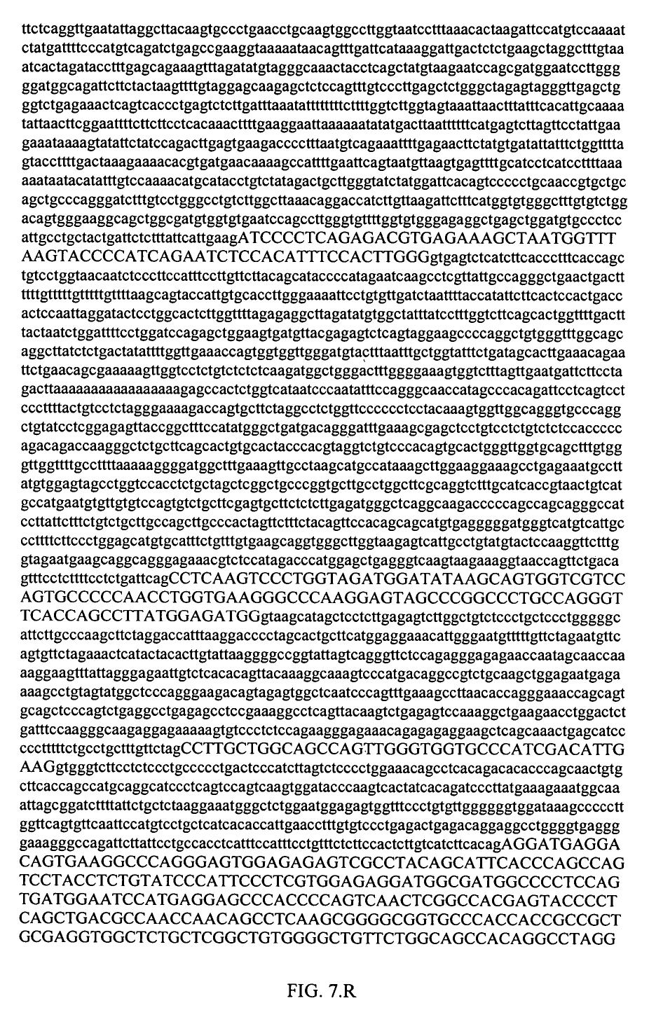

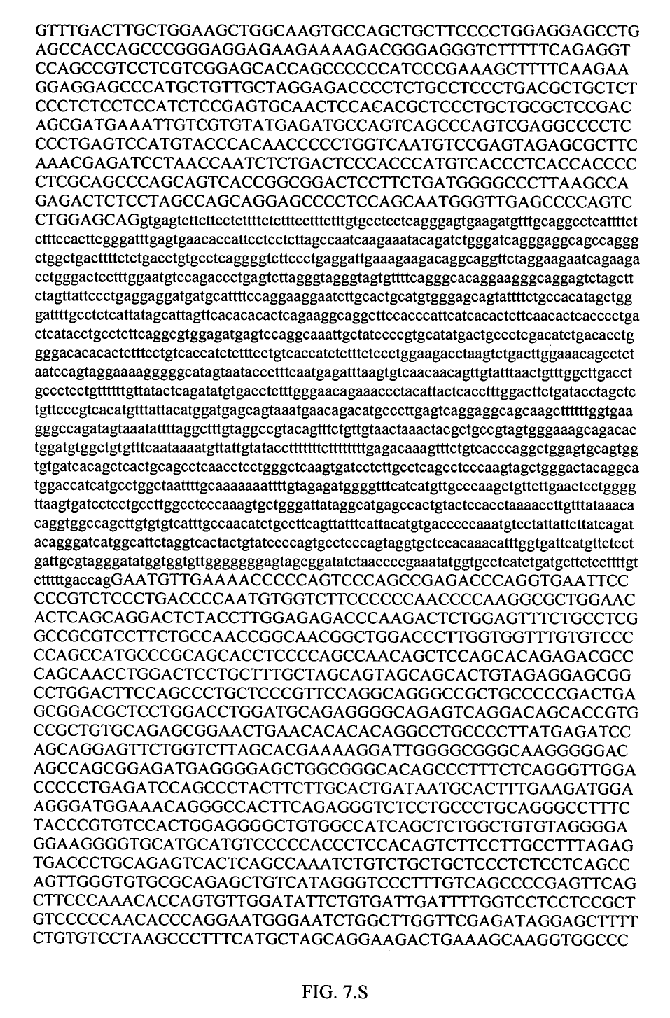

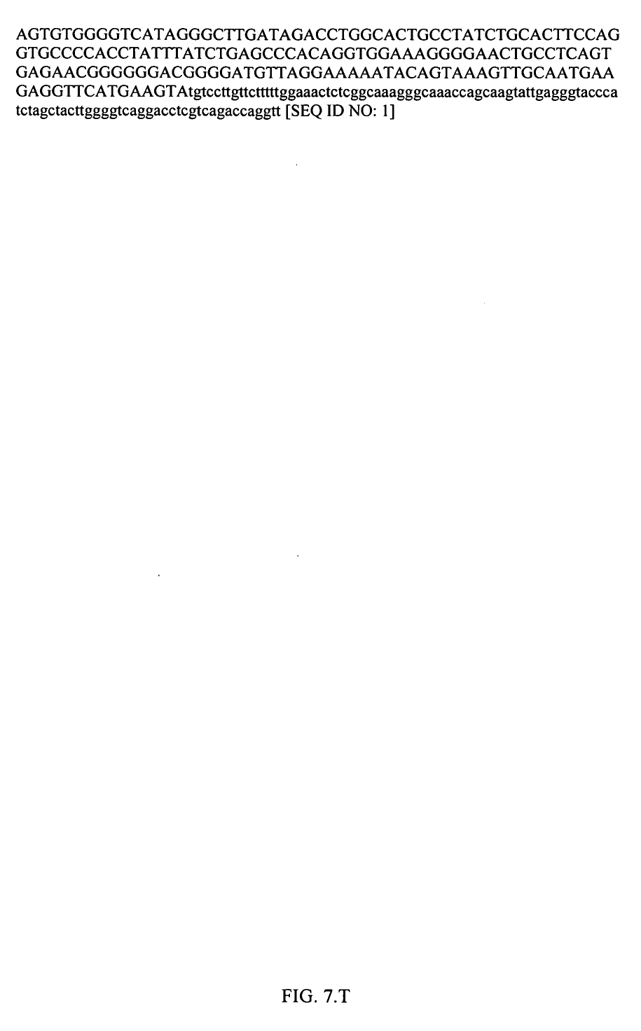

The full sequence of the MAP3K9 gene is shown in SEQ ID NO: 1 and FIGS. 7A to 7T. Additional single nucleotide polymorphisms are reported in Table 5 and may or may not be shown in SEQ ID NO: 1. It should be understood that the nucleic acids and their gene products embraced by the invention include the nucleotide sequence set forth in SEQ ID NO: 1 and may further comprise at least one polymorphism as shown in Table 5. Four novel SNPs have been identified.

Accordingly, the invention pertains to isolated nucleic acid molecules comprising human MAP3K9 nucleic acid. The term, “MAP3K9 nucleic acid,” as used herein, refers to an isolated nucleic acid molecule encoding a MAP3K9 polypeptide (e.g., a MAP3K9 gene, such as shown in SEQ ID NO: 1). The MAP3K9 nucleic acid molecules of the present invention can be RNA, for example, mRNA, or DNA, such as cDNA and genomic DNA. DNA molecules can be double-stranded or single-stranded; single stranded RNA or DNA can be the coding, or sense, strand or the non-coding, or antisense strand. The nucleic acid molecule can include all or a portion of the coding sequence of the gene and can further comprise additional non-coding sequences such as introns and non-coding 3′ and 5′ sequences (including regulatory sequences, for example).

For example, the MAP3K9 nucleic acid can be the genomic sequence shown in FIGS. 7A to 7T, or a portion or fragment of the isolated nucleic acid molecule (e.g., cDNA or the gene) that encodes MAP3K9 polypeptide.

Additionally, nucleic acid molecules of the invention can be fused to a marker sequence, for example, a sequence that encodes a polypeptide to assist in isolation or purification of the polypeptide. Such sequences include, but are not limited to, those that encode a glutathione-S-transferase (GST) fusion protein and those that encode a hemagglutinin A (HA) polypeptide marker from influenza.

An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention may be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material may be purified to essential homogeneity, for example as determined by PAGE or column chromatography such as HPLC. Preferably, an isolated nucleic acid molecule comprises at least about 50, 80 or 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 5 kb but not limited to 4 kb, 3 kb, 2 kb, 1 kb, 0.5 kb or 0.1 kb of nucleotides which flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells, as well as partially or substantially purified DNA molecules in solution. “Isolated” nucleic acid molecules also encompass in vivo and in vitro RNA transcripts of the DNA molecules of the present invention. An isolated nucleic acid molecule can include a nucleic acid molecule or nucleic acid sequence that is synthesized chemically or by recombinant means. Therefore, recombinant DNA contained in a vector is included in the definition of “isolated” as used herein. Also, isolated nucleic acid molecules include recombinant DNA molecules in heterologous organisms, as well as partially or substantially purified DNA molecules in solution. In vivo and in vitro RNA transcripts of the DNA molecules of the present invention are also encompassed by “isolated” nucleic acid sequences. Such isolated nucleic acid molecules are useful in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern or Southern blot analysis.

The present invention also pertains to nucleic acid molecules which are not necessarily found in nature but which encode a MAP3K9 polypeptide, or another splicing variant of a MAP3K9 polypeptide or polymorphic variant thereof. Thus, for example, the invention pertains to DNA molecules comprising a sequence that is different from the naturally occurring nucleotide sequence but which, due to the degeneracy of the genetic code, encode a MAP3K9 polypeptide of the present invention. The invention also encompasses nucleic acid molecules encoding portions (fragments), or encoding variant polypeptides such as analogues or derivatives of a MAP3K9 polypeptide. Such variants can be naturally occurring, such as in the case of allelic variation or single nucleotide polymorphisms, or non-naturally-occurring, such as those induced by various mutagens and mutagenic processes. Intended variations include, but are not limited to, addition, deletion and substitution of one or more nucleotides that can result in conservative or non-conservative amino acid changes, including additions and deletions. Preferably the nucleotide (and/or resultant amino acid) changes are silent or conserved; that is, they do not alter the characteristics or activity of a MAP3K9 polypeptide. In one aspect, the nucleic acid sequences are fragments that comprise one or more polymorphic microsatellite markers. In another aspect, the nucleotide sequences are fragments that comprise one or more single nucleotide polymorphisms in a MAP3K9 gene.

Other alterations of the nucleic acid molecules of the invention can include, for example, labeling, methylation, internucleotide modifications such as uncharged linkages (e.g., methyl phosphonates, phosphotriesters, phosphoamidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Also included are synthetic molecules that mimic nucleic acid molecules in the ability to bind to a designated sequence via hydrogen bonding and other chemical interactions. Such molecules include, for example, those in which peptide linkages substitute for phosphate linkages in the backbone of the molecule.

The invention also pertains to nucleic acid molecules that hybridize under high stringency hybridization conditions, such as for selective hybridization, to a nucleotide sequence described herein (e.g., nucleic acid molecules which specifically hybridize to a nucleotide sequence encoding polypeptides described herein, and, optionally, have an activity of the polypeptide). In one aspect, the invention includes variants described herein that hybridize under high stringency hybridization conditions (e.g., for selective hybridization) to a nucleotide sequence encoding an amino acid sequence or a polymorphic variant thereof. In another aspect, the variant that hybridizes under high stringency hybridizations has an activity of a MAP3K9 polypeptide.

Such nucleic acid molecules can be detected and/or isolated by specific hybridization (e.g., under high stringency conditions). “Specific hybridization,” as used herein, refers to the ability of a first nucleic acid to hybridize to a second nucleic acid in a manner such that the first nucleic acid does not hybridize to any nucleic acid other than to the second nucleic acid (e.g., when the first nucleic acid has a higher similarity to the second nucleic acid than to any other nucleic acid in a sample wherein the hybridization is to be performed). “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 70%, 75%, 85%, 90%, 95%). For example, certain high stringency conditions can be used which distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. M. et al., “Current Protocols in Molecular Biology”, John Wiley & Sons, (2001)), the entire teachings of which are incorporated by reference herein). The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90%, or at least about 95% or more identical to each other remain hybridized to one another. By varying hybridization conditions from a level of stringency at which no hybridization occurs to a level at which hybridization is first observed, conditions which will allow a given sequence to hybridize (e.g., selectively) with the most similar sequences in the sample can be determined.

Exemplary conditions are described in Krause, M. H. and S. A. Aaronson, Methods in Enzymology 200:546-556 (1991), and in, Ausubel, et al., “Current Protocols in Molecular Biology”, John Wiley & Sons, (2001), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in Tₘ of −17° C. Using these guidelines, the washing temperature can be determined empirically for high, moderate or low stringency, depending on the level of mismatch sought.

For example, a low stringency wash can comprise washing in a solution containing 0.2×SSC/0.1% SDS for 10 minutes at room temperature; a moderate stringency wash can comprise washing in a pre-warmed solution (42° C.) solution containing 0.2×SSC/0.1% SDS for 15 minutes at 42° C.; and a high stringency wash can comprise washing in pre-warmed (68° C.) solution containing 0.1×SSC/0.1% SDS for 15 minutes at 68° C. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid molecule and the primer or probe used.

The percent homology or identity of two nucleotide or amino acid sequences can be determined by aligning the sequences for optimal comparison purposes (e.g., gaps can be introduced in the sequence of a first sequence for optimal alignment). The nucleotides or amino acids at corresponding positions are then compared, and the percent identity between the two sequences is a function of the number of identical positions shared by the sequences (i.e., % identity=# of identical positions/total # of positions×100). When a position in one sequence is occupied by the same nucleotide or amino acid residue as the corresponding position in the other sequence, then the molecules are homologous at that position. As used herein, nucleic acid or amino acid “homology” is equivalent to nucleic acid or amino acid “identity”. In certain aspects, the length of a sequence aligned for comparison purposes is at least 30%, for example, at least 40%, in certain aspects at least 60%, and in other aspects at least 70%, 80%, 90% or 95% of the length of the reference sequence. The actual comparison of the two sequences can be accomplished by well-known methods, for example, using a mathematical algorithm. A preferred, non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one aspect, parameters for sequence comparison can be set at score=100, wordlength=12, or can be varied (e.g., W=5 or W=20).

Another preferred non-limiting example of a mathematical algorithm utilized for the comparison of sequences is the algorithm of Myers and Miller, CABIOS 4(1): 11-17 (1988). Such an algorithm is incorporated into the ALIGN program (version 2.0) which is part of the GCG sequence alignment software package (Accelrys, Cambridge, UK). When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, Comput. Appl. Biosci. 10:3-5 (1994); and FASTA described in Pearson and Lipman, Proc. Natl. Acad. Sci. USA 85:2444-8 (1988).

In another aspect, the percent identity between two amino acid sequences can be accomplished using the GAP program in the GCG software package using either a BLOSUM63 matrix or a PAM250 matrix, and a gap weight of 12, 10, 8, 6, or 4 and a length weight of 2, 3, or 4. In yet another aspect, the percent identity between two nucleic acid sequences can be accomplished using the GAP program in the GCG software package using a gap weight of 50 and a length weight of 3.

The present invention also provides isolated nucleic acid molecules that contain a fragment or portion that hybridizes under highly stringent conditions to a nucleotide sequence of SEQ ID NO: 1 or the complement of such a sequence, and also provides isolated nucleic acid molecules that contain a fragment or portion that hybridizes under highly stringent conditions to a nucleotide sequence encoding an amino acid sequence or polymorphic variant thereof. The nucleic acid fragments of the invention are at least about 15, preferably at least about 18, 20, 23 or 25 nucleotides, and can be 30, 40, 50, 100, 200 or more nucleotides in length. Longer fragments, for example, 30 or more nucleotides in length, that encode antigenic polypeptides described herein are particularly useful, such as for the generation of antibodies as described below.

Probes and Primers

In a related aspect, the nucleic acid fragments of the invention are used as probes or primers in assays such as those described herein. “Probes” or “primers” are oligonucleotides that hybridize in a base-specific manner to a complementary strand of nucleic acid molecules. Such probes and primers include polypeptide nucleic acids, as described in Nielsen et al., Science 254:1497-1500 (1991).

A probe or primer comprises a region of nucleotide sequence that hybridizes to at least about 15, for example about 20-25, and in certain aspects about 40, 50 or 75, consecutive nucleotides of a nucleic acid molecule comprising a contiguous nucleotide sequence of SEQ ID NO: 1 or polymorphic variant thereof. In other aspects, a probe or primer comprises 100 or fewer nucleotides, in certain aspects from 6 to 50 nucleotides, for example from 12 to 30 nucleotides. In other aspects, the probe or primer is at least 70% identical to the contiguous nucleotide sequence or to the complement of the contiguous nucleotide sequence, for example at least 80% identical, in certain aspects at least 90% identical, and in other aspects at least 95% identical, or even capable of selectively hybridizing to the contiguous nucleotide sequence or to the complement of the contiguous nucleotide sequence. Often, the probe or primer further comprises a label, e.g., radioisotope, fluorescent compound, enzyme, or enzyme co-factor.

The nucleic acid molecules of the invention such as those described above can be identified and isolated using standard molecular biology techniques and the sequence information provided herein. For example, nucleic acid molecules can be amplified and isolated by the polymerase chain reaction using synthetic oligonucleotide primers designed based on the sequence of SEQ ID NO: 1 or the complement of such a sequence, or designed based on nucleotides based on sequences encoding one or more of the amino acid sequences provided herein. See generally PCR Technology: Principles and Applications for DNA Amplification (ed. H. A. Erlich, Freeman Press, NY, N.Y., 1992); PCR Protocols: A Guide to Methods and Applications (Eds. Innis et al., Academic Press, San Diego, Calif., 1990); Mattila et al., Nuc. Acids Res. 19: 4967 (1991); Eckert et al., PCR Methods and Applications 1:17 (1991); PCR (eds. McPherson et al., IRL Press, Oxford); and U.S. Pat. No. 4,683,202. The nucleic acid molecules can be amplified using cDNA, mRNA or genomic DNA as a template, cloned into an appropriate vector and characterized by DNA sequence analysis.

Other suitable amplification methods include the ligase chain reaction (LCR) (see Wu and Wallace, Genomics 4:560 (1989), Landegren et al., Science 241:1077 (1988), transcription amplification (Kwoh et al., Proc. Natl. Acad. Sci. USA 86:1173 (1989)), and self-sustained sequence replication (Guatelli et al., Proc. Nat. Acad. Sci. USA 87:1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The amplified DNA can be labeled, for example, radiolabeled, and used as a probe for screening a cDNA library derived from human cells, mRNA in zap express, ZIPLOX or other suitable vector. Corresponding clones can be isolated, DNA can obtained following in vivo excision, and the cloned insert can be sequenced in either or both orientations by art recognized methods to identify the correct reading frame encoding a polypeptide of the appropriate molecular weight. For example, the direct analysis of the nucleotide sequence of nucleic acid molecules of the present invention can be accomplished using well-known methods that are commercially available. See, for example, Sambrook et al., Molecular Cloning, A Laboratory Manual (2nd Ed., CSHP, New York 1989); Zyskind et al., Recombinant DNA Laboratory Manual, (Acad. Press, 1988)). Additionally, fluorescence methods are also available for analyzing nucleic acids (Chen et al., Genome Res. 9, 492 (1999)) and polypeptides. Using these or similar methods, the polypeptide and the DNA encoding the polypeptide can be isolated, sequenced and further characterized.

Antisense nucleic acid molecules of the invention can be designed using the nucleotide sequence of SEQ ID NO: 1 and/or the complement or a portion, and constructed using chemical synthesis and enzymatic ligation reactions using procedures known in the art. For example, an antisense nucleic acid molecule (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid molecule can be produced biologically using an expression vector into which a nucleic acid molecule has been subcloned in an antisense orientation (i.e., RNA transcribed from the inserted nucleic acid molecule will be of an antisense orientation to a target nucleic acid of interest).

The nucleic acid sequences can also be used to compare with endogenous DNA sequences in patients to identify one or more of the disorders described above, and as probes, such as to hybridize and discover related DNA sequences or to subtract out known sequences from a sample. The nucleic acid sequences can further be used to derive primers for genetic fingerprinting, to raise anti-polypeptide antibodies using DNA immunization techniques, and as an antigen to raise anti-DNA antibodies or elicit immune responses. Portions or fragments of the nucleotide sequences identified herein (and the corresponding complete gene sequences) can be used in numerous ways, such as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Additionally, the nucleotide sequences of the invention can be used to identify and express recombinant polypeptides for analysis, characterization or therapeutic use, or as markers for tissues in which the corresponding polypeptide is expressed, either constitutively, during tissue differentiation, or in diseased states. The nucleic acid sequences can additionally be used as reagents in the screening and/or diagnostic assays described herein, and can also be included as components of kits (e.g., reagent kits) for use in the screening and/or diagnostic assays described herein.

Kits (e.g., reagent kits) useful in the methods of diagnosis comprise components useful in any of the methods described herein, including for example, hybridization probes or primers as described herein (e.g., labeled probes or primers), reagents for detection of labeled molecules, restriction enzymes (e.g., for RFLP analysis), allele-specific oligonucleotides, antibodies which bind to altered or to non-altered (native) MAP3K9 polypeptide, means for amplification of nucleic acids comprising a MAP3K9 nucleic acid, or means for analyzing the nucleic acid sequence of a MAP3K9 nucleic acid or for analyzing the amino acid sequence of a MAP3K9 polypeptide as described herein, etc. In one aspect, the kit for diagnosing a asthma or a susceptibility to asthma can comprise primers for nucleic acid amplification of a region in the MAP3K9 nucleic acid comprising an at-risk haplotype that is more frequently present in an individual having asthma or who is susceptible to asthma. The primers can be designed using portions of the nucleic acids flanking SNPs that are indicative of asthma. In a certain aspect, the primers are designed to amplify regions of the MAP3K9 gene associated with an at-risk haplotype for asthma, as shown in Table 1.

Vectors and Host Cells

Another aspect of the invention pertains to nucleic acid constructs containing a nucleic acid molecules described herein and the complements thereof (or a portion thereof). The constructs comprise a vector (e.g., an expression vector) into which a sequence of the invention has been inserted in a sense or antisense orientation. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Expression vectors are capable of directing the expression of genes to which they are operably linked. In general, expression vectors of utility in recombinant DNA techniques are often in the form of plasmids. However, the invention is intended to include such other forms of expression vectors, such as viral vectors (e.g., replication defective retroviruses, adenoviruses and adeno-associated viruses) that serve equivalent functions.

In certain aspects, recombinant expression vectors of the invention comprise a nucleic acid molecule of the invention in a form suitable for expression of the nucleic acid molecule in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” or “operatively linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, “Gene Expression Technology”, Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed and the level of expression of polypeptide desired. The expression vectors of the invention can be introduced into host cells to thereby produce polypeptides, including fusion polypeptides, encoded by nucleic acid molecules as described herein.

The recombinant expression vectors of the invention can be designed for expression of a polypeptide of the invention in prokaryotic or eukaryotic cells, e.g., bacterial cells such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example using T7 promoter regulatory sequences and T7 polymerase.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a nucleic acid molecule of the invention can be expressed in bacterial cells (e.g., E. coli), insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing a foreign nucleic acid molecule (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al., (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those that confer resistance to drugs, such as G418, hygromycin and methotrexate. Nucleic acid molecules encoding a selectable marker can be introduced into a host cell on the same vector as the nucleic acid molecule of the invention or can be introduced on a separate vector. Cells stably transfected with the introduced nucleic acid molecule can be identified by drug selection (e.g., cells that have incorporated the selectable marker gene will survive, while the other cells die).

A host cell of the invention, such as a prokaryotic or eukaryotic host cell in culture can be used to produce (i.e., express) a polypeptide of the invention. Accordingly, the invention further provides methods for producing a polypeptide using the host cells of the invention. In one aspect, the method comprises culturing the host cell of invention (into which a recombinant expression vector encoding a polypeptide of the invention has been introduced) in a suitable medium such that the polypeptide is produced. In another aspect, the method further comprises isolating the polypeptide from the medium or the host cell.

The host cells of the invention can also be used to produce nonhuman transgenic animals. For example, in one aspect, a host cell of the invention is a fertilized oocyte or an embryonic stem cell into which a nucleic acid molecule of the invention has been introduced (e.g., an exogenous MAP3K9 gene, or an exogenous nucleic acid encoding a MAP3K9 polypeptide). Such host cells can then be used to create non-human transgenic animals in which exogenous nucleotide sequences have been introduced into the genome or homologous recombinant animals in which endogenous nucleotide sequences have been altered. Such animals are useful for studying the function and/or activity of the nucleotide sequence and polypeptide encoded by the sequence and for identifying and/or evaluating modulators of their activity. As used herein, a “transgenic animal” is a non-human animal, preferably a mammal, more preferably a rodent such as a rat or mouse, in which one or more of the cells of the animal include a transgene. Other examples of transgenic animals include non-human primates, sheep, dogs, cows, goats, chickens and amphibians. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo (Cold Spring Harbor Laboratory Press, Cold Spring Harbor, N.Y., 1986). Methods for constructing homologous recombination vectors and homologous recombinant animals are described further in Bradley, Current Opinion in BioTechnology 2:823-829 (1991) and in PCT Publication Nos. WO 90/11354, WO 91/01140, WO 92/0968, and WO 93/04169. Clones of the non-human transgenic animals described herein can also be produced according to the methods described in Wilmut et al., Nature 385:810-813 (1997) and PCT Publication Nos. WO 97/07668 and WO 97/07669.

Ribonucleic Acid (RNA) of the Invention:

In one aspect, the invention relates to methods of measuring RNA levels of the MLK kinases (e.g., MLK1) using Real-Time Quantitative PCR assay in which oligo nucleotides specific for members of the MLK kinase family (e.g., MLK1) are used to amplify reverse transcribed RNA (c-DNA) on RNA samples that are isolated from blood leukocytes or other tissue samples. The method includes obtaining a sample of cells from the patient, and determining RNA levels using sequence specific probes that hybridize to PCR products of MLK kinases (e.g., MLK1) by employing the 5′→3′ exonuclease activity of Taq DNA polymerase on RNA samples that are isolated from cells that have been exposed to specific cytokine activators that activate the JNK pathway (such as IL1b or TNFα) vs vehicle alone (i.e., no activation). In one aspect, the TaqMan probe consists of a site-specific sequence labeled with a fluorescent reporter dye and a fluorescent quencher dye wherein, during the PCR reaction, the TaqMan probe hybridizes to its complementary single strand DNA sequence within the PCR target, the final level being measured by spectrophotometry after termination of the PCR. In another aspect, MLK kinase expression (e.g., MLK1) is determined using SYBR Green as a fluorescent dye. This dye fluoresces only when bound to double-stranded DNA, i.e., when MLK kinase (e.g., MLK1) uniquely designed primers bind and allow for discrimation between different RNA splice variants. In yet another aspect the invention is directed at methods that determine the role of MAP3k9 or its pathway-related genes, by obtaining a sample of cells from patients with asthma or other respiratory or inflammatory disorders, determining RNA levels of MAP3k9 or its pathway related genes in cells exposed to pathway specific activators (such as IL1b or TNFα) or vehicle alone (no activation), and comparing them with reference RNA levels of the gene in cells isolated from subjects without asthma or other inflammatory/respiratory disorders. In another aspect, the invention relates to methods for predicting efficacy of an inhibitor drug, including obtaining a sample of cells from patients with asthma or another respiratory/inflammatory disorder, determining RNA levels of MAP3k9 or its pathway related genes in cells isolated from patients who are taking the drug compared to those who are not taking the drug. In another aspect, the invention relates to methods for predicting efficacy of an inhibitor drug, including obtaining a sample of cells from patients with asthma or other respiratory/inflammatory disorders, determining RNA levels of MAP3k9 or its pathway related genes after exposure of the cells to the inhibitor drug in vitro.

Polypeptides of the Invention

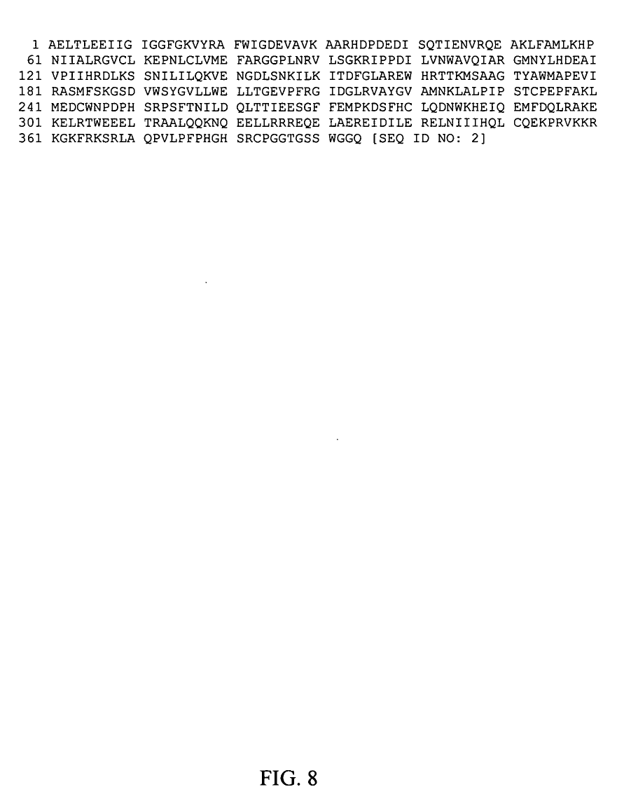

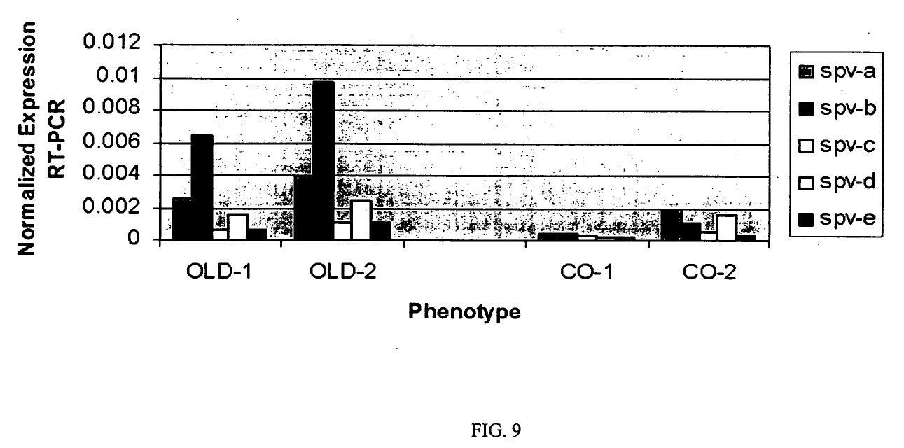

The present invention also pertains to isolated polypeptides encoded by MAP3K9 nucleic acids (“MAP3K9 polypeptides,” or “MAP3K9 proteins,” such as the protein shown in SEQ ID NO: 2, FIG. 8, FIG. 9 and NCBI accession number XM_{—}027237; (mRNA); the entire sequence being incorporated herein by reference) and fragments and variants thereof, as well as polypeptides encoded by nucleotide sequences described herein (e.g., other splicing variants). The term “polypeptide” refers to a polymer of amino acids, and not to a specific length; thus, peptides, oligopeptides and proteins are included within the definition of a polypeptide. As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell (e.g., in a “fusion protein”) and still be “isolated” or “purified.”

The polypeptides of the invention can be purified to homogeneity. It is understood, however, that preparations in which the polypeptide is not purified to homogeneity are useful. The critical feature is that the preparation allows for the desired function of the polypeptide, even in the presence of considerable amounts of other components. Thus, the invention encompasses various degrees of purity. In one aspect, the language “substantially free of cellular material” includes preparations of the polypeptide having less than about 30% (by dry weight) other proteins (i.e., contaminating protein), less than about 20% other proteins, less than about 10% other proteins, or less than about 5% other proteins.

When a polypeptide is recombinantly produced, it can also be substantially free of culture medium, i.e., culture medium represents less than about 20%, less than about 10%, or less than about 5% of the volume of the polypeptide preparation. The language “substantially free of chemical precursors or other chemicals” includes preparations of the polypeptide in which it is separated from chemical precursors or other chemicals that are involved in its synthesis. In one aspect, the language “substantially free of chemical precursors or other chemicals” includes preparations of the polypeptide having less than about 30% (by dry weight) chemical precursors or other chemicals, less than about 20% chemical precursors or other chemicals, less than about 10% chemical precursors or other chemicals, or less than about 5% chemical precursors or other chemicals.

In one aspect, a polypeptide of the invention comprises an amino acid sequence encoded by a nucleic acid molecule comprising a nucleotide sequence of SEQ ID NO: 1, or the complement of such a nucleic acid, or portions thereof, or a portion or polymorphic variant thereof. However, the polypeptides of the invention also encompass fragment and sequence variants. Variants include a substantially homologous polypeptide encoded by the same genetic locus in an organism, i.e., an allelic variant, as well as other splicing variants. Variants also encompass polypeptides derived from other genetic loci in an organism, but having substantial homology to a polypeptide encoded by a nucleic acid molecule comprising a nucleotide of SEQ ID NO: 1 or a complement of such a sequence, or portions thereof or polymorphic variants thereof. Variants also include polypeptides substantially homologous or identical to these polypeptides but derived from another organism, i.e., an ortholog. Variants also include polypeptides that are substantially homologous or identical to these polypeptides that are produced by chemical synthesis. Variants also include polypeptides that are substantially homologous or identical to these polypeptides that are produced by recombinant methods.

As used herein, two polypeptides (or a region of the polypeptides) are substantially homologous or identical when the amino acid sequences are at least about 45-55%, in certain aspects at least about 70-75%, and in other aspects at least about 80-85%, and in other aspects greater than about 90% or more homologous or identical. A substantially homologous amino acid sequence, according to the present invention, will be encoded by a nucleic acid molecule hybridizing to a nucleic acid of the invention or portion thereof or polymorphic variant thereof, under stringent conditions as more particularly described above.

The invention also encompasses polypeptides having a lower degree of identity but having sufficient similarity so as to perform one or more of the same functions performed by a polypeptide encoded by a nucleic acid molecule of the invention.

Similarity is determined by conserved amino acid substitution where a given amino acid in a polypeptide is substituted by another amino acid of like characteristics. Conservative substitutions are likely to be phenotypically silent. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr. Guidance concerning which amino acid changes are likely to be phenotypically silent are found in Bowie et al., Science 247:1306-1310 (1990).

A variant polypeptide can differ in amino acid sequence by one or more substitutions, deletions, insertions, inversions, fusions, and truncations or a combination of any of these. Further, variant polypeptides can be fully functional or can lack function in one or more activities. Fully functional variants typically contain only conservative variation or variation in non-critical residues or in non-critical regions. Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncation or a substitution, insertion, inversion, or deletion in a critical residue or critical region.

Amino acids that are essential for function can be identified by methods known in the art, such as site-directed mutagenesis or alanine-scanning mutagenesis (Cunningham et al., Science 244:1082-1185 (1989)). The latter procedure introduces single alanine mutations at every residue in the molecule. The resulting mutant molecules are then tested for biological activity in vitro, or in vitro proliferative activity. Sites that are critical for polypeptide activity can also be determined by structural analysis such as crystallization, nuclear magnetic resonance or photoaffinity labeling (Smith et al., J. Mol. Biol. 224:899-904 (1992); de Vos et al., Science 255:306-312 (1992)).

The invention also includes polypeptide fragments of the polypeptides of the invention. Fragments can be derived from a polypeptide encoded by a nucleic acid molecule comprising SEQ ID NO: 1 or a complement of such a nucleic acid or other variants. However, the invention also encompasses fragments of the variants of the polypeptides described herein. As used herein, a fragment comprises at least 6 contiguous amino acids. Useful fragments include those that retain one or more of the biological activities of the polypeptide as well as fragments that can be used as an immunogen to generate polypeptide-specific antibodies.

Biologically active fragments (peptides which are, for example, 6, 9, 12, 15, 16, 20, 30, 35, 36, 37, 38, 39, 40, 50, 100 or more amino acids in length) can comprise a domain, segment, or motif that has been identified by analysis of the polypeptide sequence using well-known methods, e.g., signal peptides, extracellular domains, one or more transmembrane segments or loops, ligand binding regions, zinc finger domains, DNA binding domains, acylation sites, glycosylation sites, or phosphorylation sites.

Fragments can be discrete (not fused to other amino acids or polypeptides) or can be within a larger polypeptide. Further, several fragments can be comprised within a single larger polypeptide. In one aspect a fragment designed for expression in a host can have heterologous pre- and pro-polypeptide regions fused to the amino terminus of the polypeptide fragment and an additional region fused to the carboxyl terminus of the fragment.

The invention thus provides chimeric or fusion polypeptides. These comprise a polypeptide of the invention operatively linked to a heterologous protein or polypeptide having an amino acid sequence not substantially homologous to the polypeptide.

“Operatively linked” indicates that the polypeptide and the heterologous protein are fused in-frame. The heterologous protein can be fused to the N-terminus or C-terminus of the polypeptide. In one aspect the fusion polypeptide does not affect function of the polypeptide per se. For example, the fusion polypeptide can be a GST-fusion polypeptide in which the polypeptide sequences are fused to the C-terminus of the GST sequences. Other types of fusion polypeptides include, but are not limited to, enzymatic fusion polypeptides, for example beta-galactosidase fusions, yeast two-hybrid hybrid GAL fusions, poly-His fusions and Ig fusions. Such fusion polypeptides, particularly poly-His fusions, can facilitate the purification of recombinant polypeptide. In certain host cells (e.g., mammalian host cells), expression and/or secretion of a polypeptide can be increased using a heterologous signal sequence. Therefore, in another aspect, the fusion polypeptide contains a heterologous signal sequence at its N-terminus.

EP-A-O 464 533 discloses fusion proteins comprising various portions of immunoglobulin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists. Bennett et al., Journal of Molecular Recognition, 8:52-58 (1995) and Johanson et al., The Journal of Biological Chemistry, 270,16:9459-9471 (1995). Thus, this invention also encompasses soluble fusion polypeptides containing a polypeptide of the invention and various portions of the constant regions of heavy or light chains of immunoglobulins of various subclasses (IgG, IgM, IgA, IgE).

A chimeric or fusion polypeptide can be produced by standard recombinant DNA techniques. For example, DNA fragments coding for the different polypeptide sequences are ligated together in-frame in accordance with conventional techniques. In another aspect, the fusion gene can be synthesized by conventional techniques including automated DNA synthesizers. Alternatively, PCR amplification of nucleic acid fragments can be carried out using anchor primers which give rise to complementary overhangs between two consecutive nucleic acid fragments which can subsequently be annealed and re-amplified to generate a chimeric nucleic acid sequence (see Ausubel et al., Current Protocols in Molecular Biology, 1992).

Moreover, many expression vectors are commercially available that already encode a fusion moiety (e.g., a GST protein). A nucleic acid molecule encoding a polypeptide of the invention can be cloned into such an expression vector such that the fusion moiety is linked in-frame to the polypeptide.

The isolated polypeptide can be purified from cells that naturally express it, can be purified from cells that have been altered to express it (recombinant), or synthesized using known protein synthesis methods. In one aspect, the polypeptide is produced by recombinant DNA techniques. For example, a nucleic acid molecule encoding the polypeptide is cloned into an expression vector, the expression vector introduced into a host cell and the polypeptide expressed in the host cell. The polypeptide can then be isolated from the cells by an appropriate purification scheme using standard protein purification techniques.

The polypeptides of the present invention can be used to raise antibodies or to elicit an immune response. The polypeptides can also be used as a reagent, e.g., a labeled reagent, in assays to quantitatively determine levels of the polypeptide or a molecule to which it binds (e.g., a ligand) in biological fluids. The polypeptides can also be used as markers for cells or tissues in which the corresponding polypeptide is preferentially expressed, either constitutively, during tissue differentiation, or in a diseased state. The polypeptides can be used to isolate a corresponding binding agent, e.g., ligand or receptor, such as, for example, in an interaction trap assay, and to screen for peptide or small molecule antagonists or agonists of the binding interaction.

### ANTIBODIES OF THE INVENTION

Polyclonal antibodies and/or monoclonal antibodies that specifically bind one form of the gene product but not to the other form of the gene product are also provided. Antibodies are also provided which bind a portion of either the variant or the reference gene product that contains the polymorphic site or sites. The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4: 72 (1983)), the EBV-hybridoma technique (Cole et al., Monoclonal Antibodies and Cancer Therapy, Alan R. Liss, 1985, Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9: 1370-1372 (1991); Hay et al., Hum. Antibod. Hybridomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Diagnostic Assays

The nucleic acids, probes, primers, polypeptides and antibodies described herein can be used in methods of diagnosis of asthma; of a susceptibility to asthma; or of a condition associated with a MAP3K9 gene, as well as in kits (e.g., useful for diagnosis of asthma; a susceptibility to asthma; or a condition associated with a MAP3K9 gene). In one aspect, the kit comprises primers that can be used to amplify the markers of interest.

In one aspect of the invention, diagnosis of a disease or condition associated with a MAP3K9 gene (e.g., diagnosis of asthma, or of a susceptibility to asthma) is made by detecting a polymorphism in a MAP3K9 nucleic acid as described herein. The polymorphism can be a change in a MAP3K9 nucleic acid, such as the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of the gene; duplication of all or a part of the gene; transposition of all or a part of the gene; or rearrangement of all or a part of the gene. More than one such change may be present in a single gene. Such sequence changes cause a difference in the polypeptide encoded by a MAP3K9 nucleic acid. For example, if the difference is a frame shift change, the frame shift can result in a change in the encoded amino acids, and/or can result in the generation of a premature stop codon, causing generation of a truncated polypeptide. Alternatively, a polymorphism associated with a disease or condition or a susceptibility to a disease or condition associated with a MAP3K9 nucleic acid can be a synonymous alteration in one or more nucleotides (i.e., an alteration that does not result in a change in the polypeptide encoded by a MAP3K9 nucleic acid). Such a polymorphism may alter splicing sites, affect the stability or transport of mRNA, or otherwise affect the transcription or translation of the gene. A MAP3K9 nucleic acid that has any of the changes or alterations described above is referred to herein as an “altered nucleic acid.”

In a first method of diagnosing asthma or a susceptibility to asthma, or another disease or condition associated with a MAP3K9 gene, hybridization methods, such as Southern analysis, Northern analysis, or in situ hybridizations, can be used (see Current Protocols in Molecular Biology, Ausubel, F. et al., eds, John Wiley & Sons, including all supplements through 1999). For example, a biological sample (a “test sample”) from a test subject (the “test individual”) of genomic DNA, RNA, or cDNA, is obtained from an individual, such as an individual suspected of having, being susceptible to or predisposed for, or carrying a defect for, the disease or condition, or the susceptibility to the disease or condition, associated with a MAP3K9 gene (e.g., asthma). The individual can be an adult, child, or fetus. The test sample can be from any source which contains genomic DNA, such as a blood sample, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined to determine whether a polymorphism in a MAP3K9 nucleic acid is present, and/or to determine which splicing variant(s) encoded by the MAP3K9 is present. The presence of the polymorphism or splicing variant(s) can be indicated by hybridization of the gene in the genomic DNA, RNA, or cDNA to a nucleic acid probe. A “nucleic acid probe”, as used herein, can be a DNA probe or an RNA probe; the nucleic acid probe can contain, for example, at least one polymorphism in a MAP3K9 nucleic acid (e.g., as set forth in Table 2) and/or contain a nucleic acid encoding a particular splicing variant of a MAP3K9 nucleic acid. The probe can be any of the nucleic acid molecules described above (e.g., the gene or nucleic acid, a fragment, a vector comprising the gene or nucleic acid, a probe or primer, etc.).

To diagnose asthma, or a susceptibility to asthma, or another condition associated with a MAP3K9 gene, a hybridization sample is formed by contacting the test sample containing a MAP3K9 nucleic acid with at least one nucleic acid probe. A preferred probe for detecting mRNA or genomic DNA is a labeled nucleic acid probe capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate mRNA or genomic DNA. For example, the nucleic acid probe can be all or a portion of one of SEQ ID NOs: 4-109 or the complement thereof, or a portion thereof. Other suitable probes for use in the diagnostic assays of the invention are described above (see e.g., probes and primers discussed under the heading, “Nucleic Acids of the Invention”).

The hybridization sample is maintained under conditions that are sufficient to allow specific hybridization of the nucleic acid probe to a MAP3K9 nucleic acid. “Specific hybridization”, as used herein, indicates exact hybridization (e.g., with no mismatches). Specific hybridization can be performed under high stringency conditions or moderate stringency conditions, for example, as described above. In a particularly preferred aspect, the hybridization conditions for specific hybridization are high stringency.

Specific hybridization, if present, is then detected using standard methods. If specific hybridization occurs between the nucleic acid probe and MAP3K9 nucleic acid in the test sample, then the MAP3K9 has the polymorphism, or is the splicing variant, that is present in the nucleic acid probe. More than one nucleic acid probe can also be used concurrently in this method. Specific hybridization of any one of the nucleic acid probes is indicative of a polymorphism in the MAP3K9 nucleic acid, or of the presence of a particular splicing variant encoding the MAP3K9 nucleic acid and is therefore diagnostic for a susceptibility to a disease or condition associated with a MAP3K9 nucleic acid (e.g., asthma).

In Northern analysis (see Current Protocols in Molecular Biology, Ausubel, F. et al., eds., John Wiley & Sons, supra) the hybridization methods described above are used to identify the presence of a polymorphism or a particular splicing variant, associated with a susceptibility to a disease or condition associated with a MAP3K9 gene (e.g., asthma). For Northern analysis, a test sample of RNA is obtained from the individual by appropriate means. Specific hybridization of a nucleic acid probe, as described above, to RNA from the individual is indicative of a polymorphism in a MAP3K9 nucleic acid, or of the presence of a particular splicing variant encoded by a MAP3K9 nucleic acid and is therefore diagnostic for asthma or a susceptibility to asthma or a condition associated with a MAP3K9 nucleic acid (e.g., asthma).

For representative examples of use of nucleic acid probes, see, for example, U.S. Pat. Nos. 5,288,611 and 4,851,330.

Alternatively, a peptide nucleic acid (PNA) probe can be used instead of a nucleic acid probe in the hybridization methods described above. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P. E. et al., Bioconjugate Chemistry 5, American Chemical Society, p. 1 (1994). The PNA probe can be designed to specifically hybridize to a gene having a polymorphism associated with a susceptibility to a disease or condition associated with a MAP3K9 nucleic acid (e.g., asthma). Hybridization of the PNA probe to a MAP3K9 gene is diagnostic for asthma or a susceptibility to asthma or a condition associated with a MAP3K9 nucleic acid.

In another method of the invention, alteration analysis by restriction digestion can be used to detect an altered gene, or genes containing a polymorphism(s), if the alteration (mutation) or polymorphism in the gene results in the creation or elimination of a restriction site. A test sample containing genomic DNA is obtained from the individual. Polymerase chain reaction (PCR) can be used to amplify a MAP3K9 nucleic acid (and, if necessary, the flanking sequences) in the test sample of genomic DNA from the test individual. RFLP analysis is conducted as described (see Current Protocols in Molecular Biology, supra). The digestion pattern of the relevant DNA fragment indicates the presence or absence of the alteration or polymorphism in the MAP3K9 nucleic acid, and therefore indicates the presence or absence of asthma or the susceptibility to a disease or condition associated with a MAP3K9 nucleic acid.

Sequence analysis can also be used to detect specific polymorphisms in a MAP3K9 nucleic acid. A test sample of DNA or RNA is obtained from the test individual. PCR or other appropriate methods can be used to amplify the gene or nucleic acid, and/or its flanking sequences, if desired. The sequence of a MAP3K9 nucleic acid, or a fragment of the nucleic acid, or cDNA, or fragment of the cDNA, or mRNA, or fragment of the mRNA, is determined, using standard methods. The sequence of the nucleic acid, nucleic acid fragment, cDNA, cDNA fragment, mRNA, or mRNA fragment is compared with the known nucleic acid sequence of the gene or cDNA or mRNA, as appropriate. The presence of a polymorphism in the MAP3K9 indicates that the individual has asthma or a susceptibility to asthma.

Allele-specific oligonucleotides can also be used to detect the presence of a polymorphism in a MAP3K9 nucleic acid, through the use of dot-blot hybridization of amplified oligonucleotides with allele-specific oligonucleotide (ASO) probes (see, for example, Saiki, R. et al., Nature 324:163-166 (1986)). An “allele-specific oligonucleotide” (also referred to herein as an “allele-specific oligonucleotide probe”) is an oligonucleotide of approximately 10-50 base pairs, preferably approximately 15-30 base pairs, that specifically hybridizes to a MAP3K9 nucleic acid, and that contains a polymorphism associated with a susceptibility to a disease or condition associated with a MAP3K9 nucleic acid. An allele-specific oligonucleotide probe that is specific for particular polymorphisms in a MAP3K9 nucleic acid can be prepared, using standard methods (see Current Protocols in Molecular Biology, supra). To identify polymorphisms in the gene that are associated with a disease or condition associated with a MAP3K9 nucleic acid or a susceptibility to a disease or condition associated with a MAP3K9 nucleic acid a test sample of DNA is obtained from the individual. PCR can be used to amplify all or a fragment of a MAP3K9 nucleic acid and its flanking sequences. The DNA containing the amplified MAP3K9 nucleic acid (or fragment of the gene or nucleic acid) is dot-blotted, using standard methods (see Current Protocols in Molecular Biology, supra), and the blot is contacted with the oligonucleotide probe. The presence of specific hybridization of the probe to the amplified MAP3K9 nucleic acid is then detected. Hybridization of an allele-specific oligonucleotide probe to DNA from the individual is indicative of a polymorphism in the MAP3K9 nucleic acid, and is therefore indicative of a disease or condition associated with a MAP3K9 nucleic acid or susceptibility to a disease or condition associated with a MAP3K9 nucleic acid (e.g., asthma).

The invention further provides allele-specific oligonucleotides that hybridize to the reference or variant allele of a gene or nucleic acid comprising a single nucleotide polymorphism or to the complement thereof. These oligonucleotides can be probes or primers.

An allele-specific primer hybridizes to a site on target DNA overlapping a polymorphism and only primes amplification of an allelic form to which the primer exhibits perfect complementarity. See Gibbs, Nucleic Acid Res. 17, 2427-2448 (1989). This primer is used in conjunction with a second primer, which hybridizes at a distal site. Amplification proceeds from the two primers, resulting in a detectable product, which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

With the addition of such analogs as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures of 64° C. and 74° C. when in complex with complementary DNA or RNA, respectively, as oposed to 28° C. for both DNA and RNA for the corresponding DNA nonamer. Substantial increases in Tₘ are also obtained when LNA monomers are used in combination with standard DNA or RNA monomers. For primers and probes, depending on where the LNA monomers are included (e.g., the 3′ end, the 5′end, or in the middle), the Tₘ could be increased considerably.

In another aspect, arrays of oligonucleotide probes that are complementary to target nucleic acid sequence segments from an individual, can be used to identify polymorphisms in a MAP3K9 nucleic acid. For example, in one aspect, an oligonucleotide array can be used. Oligonucleotide arrays typically comprise a plurality of different oligonucleotide probes that are coupled to a surface of a substrate in different known locations. These oligonucleotide arrays, also described as “Genechips™,” have been generally described in the art, for example, U.S. Pat. No. 5,143,854 and PCT patent publication Nos. WO 90/15070 and 92/10092. These arrays can generally be produced using mechanical synthesis methods or light directed synthesis methods that incorporate a combination of photolithographic methods and solid phase oligonucleotide synthesis methods. See Fodor et al., Science 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186, the entire teachings of each of which are incorporated by reference herein. Techniques for the synthesis of these arrays using mechanical synthesis methods are described in, e.g., U.S. Pat. No. 5,384,261; the entire teachings of which are incorporated by reference herein. In another example, linear arrays can be utilized.

Once an oligonucleotide array is prepared, a nucleic acid of interest is hybridized with the array and scanned for polymorphisms. Hybridization and scanning are generally carried out by methods described herein and also in, e.g., published PCT Application Nos. WO 92/10092 and WO 95/11995, and U.S. Pat. No. 5,424,186, the entire teachings of which are incorporated by reference herein. In brief, a target nucleic acid sequence that includes one or more previously identified polymorphic markers is amplified by well-known amplification techniques, e.g., PCR. Typically, this involves the use of primer sequences that are complementary to the two strands of the target sequence both upstream and downstream from the polymorphism. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detecting a single polymorphism, arrays can include multiple detection blocks, and thus be capable of analyzing multiple, specific polymorphisms. In alternative aspects,it will generally be understood that detection blocks may be grouped within a single array or in multiple, separate arrays so that varying, optimal conditions may be used during the hybridization of the target to the array. For example, it may often be desirable to provide for the detection of those polymorphisms that fall within G-C rich stretches of a genomic sequence, separately from those falling in A-T rich segments. This allows for the separate optimization of hybridization conditions for each situation.

Additional uses of oligonucleotide arrays for polymorphism detection can be found, for example, in U.S. Pat. Nos. 5,858,659 and 5,837,832, the entire teachings of which are incorporated by reference herein. Other methods of nucleic acid analysis can be used to detect polymorphisms in a asthma gene or variants encoding by a asthma gene. Representative methods include direct manual sequencing (Church and Gilbert, Proc. Natl. Acad. Sci. USA 81:1991-1995 (1988); Sanger, F. et al., Proc. Natl. Acad. Sci. USA 74:5463-5467 (1977); Beavis et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V. C. et al., Proc. Natl. Acad. Sci. USA 86:232-236 (1989)), mobility shift analysis (Orita, M. et al., Proc. Natl. Acad. Sci. USA 86:2766-2770 (1989)), restriction enzyme analysis (Flavell et al., Cell 15:25 (1978); Geever, et al., Proc. Natl. Acad. Sci. USA 78:5081 (1981)); heteroduplex analysis; chemical mismatch cleavage (CMC) (Cotton et al., Proc. Natl. Acad. Sci. USA 85:4397-4401 (1985)); RNase protection assays (Myers, R. M. et al., Science 230:1242 (1985)); use of polypeptides which recognize nucleotide mismatches, such as E. coli mutS protein; allele-specific PCR, for example.

In one aspect of the invention, diagnosis of a disease or condition associated with a MAP3K9 nucleic acid (e.g., asthma) or a susceptibility to a disease or condition associated with a MAP3K9 nucleic acid (e.g., asthma) can also be made by expression analysis by quantitative PCR (kinetic thermal cycling). This technique, utilizing TaqMan® assays, can assess the presence of an alteration in the expression or composition of the polypeptide encoded by a MAP3K9 nucleic acid or splicing variants encoded by a MAP3K9 nucleic acid. TaqMan® probes can also be used to allow the identification of polymorphisms and whether a patient is homozygous or heterozygous. Further, the expression of the variants can be quantified as physically or functionally different.

In another aspect of the invention, diagnosis of asthma or a susceptibility to asthma or a condition associated with a MAP3K9 gene) can be made by examining expression and/or composition of a MAP3K9 polypeptide, by a variety of methods, including enzyme linked immunosorbent assays (ELISAs), Western blots, immunoprecipitations and immunofluorescence. A test sample from an individual is assessed for the presence of an alteration in the expression and/or an alteration in composition of the polypeptide encoded by a MAP3K9 nucleic acid, or for the presence of a particular variant encoded by a MAP3K9 nucleic acid. An alteration in expression of a polypeptide encoded by a MAP3K9 nucleic acid can be, for example, an alteration in the quantitative polypeptide expression (i.e., the amount of polypeptide produced); an alteration in the composition of a polypeptide encoded by a MAP3K9 nucleic acid is an alteration in the qualitative polypeptide expression (e.g., expression of an altered MAP3K9 polypeptide or of a different splicing variant). In a preferred aspect, diagnosis of the disease or condition associated with MAP3K9 nucleic acid or a susceptibility to a disease or condition associated with a MAP3K9 nucleic acid is made by detecting a particular splicing variant encoded by that MAP3K9 nucleic acid, or a particular pattern of splicing variants.

Both such alterations (quantitative and qualitative) can also be present. The term “alteration” in the polypeptide expression or composition, as used herein, refers to an alteration in expression or composition in a test sample, as compared with the expression or composition of polypeptide by a MAP3K9 nucleic acid in a control sample. A control sample is a sample that corresponds to the test sample (e.g., is from the same type of cells), and is from an individual who is not affected by a susceptibility to a disease or condition associated with a MAP3K9 nucleic acid. An alteration in the expression or composition of the polypeptide in the test sample, as compared with the control sample, is indicative of a susceptibility to a disease or condition associated with a MAP3K9 nucleic acid. Similarly, the presence of one or more different splicing variants in the test sample, or the presence of significantly different amounts of different splicing variants in the test sample, as compared with the control sample, is indicative of a disease or condition associated with a MAP3K9 nucleic acid or a susceptibility to a disease or condition associated with a MAP3K9 nucleic acid. Various means of examining expression or composition of the polypeptide encoded by a MAP3K9 nucleic acid can be used, including: spectroscopy, colorimetry, electrophoresis, isoelectric focusing, and immunoassays (e.g., David et al., U.S. Pat. No. 4,376,110) such as immunoblotting (see also Current Protocols in Molecular Biology, particularly Chapter 10). For example, in one aspect, an antibody capable of binding to the polypeptide (e.g., as described above), preferably an antibody with a detectable label, can be used. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling a DNA probe with biotin such that it can be detected with fluorescently labeled streptavidin.

Western blotting analysis, using an antibody as described above that specifically binds to a polypeptide encoded by an altered MAP3K9 nucleic acid or an antibody that specifically binds to a polypeptide encoded by a non-altered nucleic acid, or an antibody that specifically binds to a particular splicing variant encoded by a nucleic acid, can be used to identify the presence in a test sample of a particular splicing variant or of a polypeptide encoded by a polymorphic or altered MAP3K9 nucleic acid, or the absence in a test sample of a particular splicing variant or of a polypeptide encoded by a non-polymorphic or non-altered nucleic acid. The presence of a polypeptide encoded by a polymorphic or altered nucleic acid, or the absence of a polypeptide encoded by a non-polymorphic or non-altered nucleic acid, is diagnostic for a disease or condition associated with a MAP3K9 nucleic acid or a susceptibility to a disease or condition associated with a MAP3K9 nucleic acid (e.g., asthma), as is the presence (or absence) of particular splicing variants encoded by the MAP3K9 nucleic acid.

In one aspect of this method, the level or amount of polypeptide encoded by a MAP3K9 nucleic acid in a test sample is compared with the level or amount of the polypeptide encoded by the MAP3K9 in a control sample. A level or amount of the polypeptide in the test sample that is higher or lower than the level or amount of the polypeptide in the control sample, such that the difference is statistically significant, is indicative of an alteration in the expression of the polypeptide encoded by the MAP3K9 nucleic acid, and is diagnostic for a disease or condition associated with a MAP3K9 nucleic acid or a susceptibility to a disease or condition associated with that MAP3K9 nucleic acid (e.g., asthma). Alternatively, the composition of the polypeptide encoded by a MAP3K9 nucleic acid in a test sample is compared with the composition of the polypeptide encoded by the MAP3K9 nucleic acid in a control sample (e.g., the presence of different splicing variants). A difference in the composition of the polypeptide in the test sample, as compared with the composition of the polypeptide in the control sample, is diagnostic for a disease or condition associated with a MAP3K9 nucleic acid or a susceptibility to a disease or condition associated with that MAP3K9 nucleic acid (e.g., asthma). In another aspect, both the level or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of a disease or condition associated with a MAP3K9 nucleic acid or a susceptibility to a disease or condition associated with that MAP3K9 nucleic acid.

The invention further pertains to a method for the diagnosis or identification of a susceptibility to asthma in an individual, by identifying an at-risk haplotype (e.g., a haplotype comprising a MAP3K9 nucleic acid). The MAP3K9-associated haplotypes, e.g., those described in the Example section, describe a set of genetic markers (“alleles”). In a certain aspect, the haplotype can comprise one or more alleles, two or more alleles, three or more alleles, four or more alleles, or five or more alleles. The genetic markers are particular “alleles” at “polymorphic sites” associated with MAP3K9. A nucleotide position at which more than one sequence is possible in a population (either a natural population or a synthetic population, e.g., a library of synthetic molecules), is referred to herein as a “polymorphic site”. Where a polymorphic site is a single nucleotide in length, the site is referred to as a single nucleotide polymorphism (“SNP”). For example, if at a particular chromosomal location, one member of a population has an adenine and another member of the population has a thymine at the same position, then this position is a polymorphic site, and, more specifically, the polymorphic site is a SNP. Polymorphic sites can allow for differences in sequences based on substitutions, insertions or deletions. Each version of the sequence with respect to the polymorphic site is referred to herein as an “allele” of the polymorphic site. Thus, in the previous example, the SNP allows for both an adenine allele and a thymine allele.

Typically, a reference sequence is referred to for a particular sequence. Alleles that differ from the reference are referred to as “variant” alleles. For example, the reference MAP3K9 sequence is described herein by SEQ ID NO: 1. The term, “variant MAP3K9”, as used herein, refers to a sequence that differs from SEQ ID NO: 1 but is otherwise substantially similar. The genetic markers that make up the haplotypes described herein are MAP3K9 variants. The variants of MAP3K9 that are used to determine the haplotypes disclosed herein of the present invention are associated with asthma or a susceptibility to asthma.

Additional variants can include changes that affect a polypeptide, e.g., the MAP3K9 polypeptide. These sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by a MAP3K9 nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with asthma or a susceptibility to asthma can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of the polypeptide. The polypeptide encoded by the reference nucleotide sequence is the “reference” polypeptide with a particular reference amino acid sequence, and polypeptides encoded by variant alleles are referred to as “variant” polypeptides with variant amino acid sequences.

Haplotypes are a combination of genetic markers, e.g., particular alleles at polymorphic sites. The haplotypes described herein, e.g., having markers such as those shown herein, are found more frequently in individuals with asthma than in individuals without asthma. Therefore, these haplotypes have predictive value for detecting asthma or a susceptibility to asthma in an individual. The haplotypes described herein are a combination of various genetic markers, e.g., SNPs and microsatellites. Therefore, detecting haplotypes can be accomplished by methods known in the art for detecting sequences at polymorphic sites, such as the methods described above.

RNA Expression Levels:

In one aspect, the invention relates to methods of measuring RNA levels of the MLK kinases (e.g., MLK1) using Real-Time Quantitative PCR. The method includes obtaining a sample of cells from the patient, and determining RNA expression levels using sequence specific probes that hybridize to PCR products of MLK kinases (e.g., MLK1) on RNA samples that are isolated from cells that have been exposed to specific cytokine activators that activate the JNK pathway (such as IL1b or TNFα) vs vehicle alone (i.e., no activation). In another aspect the invention is directed at methods that determine the role of MAP3k9 or its pathway-related genes, by obtaining a sample of cells from patients with asthma or other respiratory or inflammatory disorders, determining RNA levels of MAP3k9 or its pathway related genes in cells exposed to pathway specific activators (such as IL1b or TNFα) or vehicle alone (no activation), and comparing them with reference RNA levels of the gene in cells isolated from subjects without asthma or other inflammatory/respiratory disorders. In another aspect, the invention relates to methods for predicting efficacy of an inhibitor drug, including obtaining a sample of cells from patients with asthma or another respiratory/inflammatory disorder, determining RNA levels of MAP3k9 or its pathway related genes in cells isolated from patients who are taking the drug compared to those who are not taking the drug. In another aspect, the invention relates to methods for predicting efficacy of an inhibitor drug, including obtaining a sample of cells from patients with asthma or other respiratory/inflammatory disorders, determining RNA levels of MAP3k9 or its pathway related genes after exposure of the cells to the inhibitor drug in vitro.

Screening Assays and Agents Identified Thereby

The invention provides methods (also referred to herein as “screening assays”) for identifying the presence of a nucleotide that hybridizes to a nucleic acid of the invention, as well as for identifying the presence of a polypeptide encoded by a nucleic acid of the invention. In one aspect, the presence (or absence) of a nucleic acid molecule of interest (e.g., a nucleic acid that has significant homology with a nucleic acid of the invention) in a sample can be assessed by contacting the sample with a nucleic acid comprising a nucleic acid of the invention under stringent conditions as described above, and then assessing the sample for the presence (or absence) of hybridization. In one aspect, high stringency conditions are conditions appropriate for selective hybridization. In another aspect, a sample containing the nucleic acid molecule of interest is contacted with a nucleic acid containing a contiguous nucleotide sequence (e.g., a primer or a probe as described above) that is at least partially complementary to a part of the nucleic acid molecule of interest (e.g., a MAP3K9 nucleic acid), and the contacted sample is assessed for the presence or absence of hybridization. In another aspect, the nucleic acid containing a contiguous nucleotide sequence is completely complementary to a part of the nucleic acid molecule of interest.

In any of these aspects, all or a portion of the nucleic acid of interest can be subjected to amplification prior to performing the hybridization.

In another aspect, the presence (or absence) of a polypeptide of interest, such as a polypeptide of the invention or a fragment or variant thereof, in a sample can be assessed by contacting the sample with an antibody that specifically hybridizes to the polypeptide of interest (e.g., an antibody such as those described above), and then assessing the sample for the presence (or absence) of binding of the antibody to the polypeptide of interest.

In another aspect, the invention provides methods for identifying agents (e.g., fusion proteins, polypeptides, peptidomimetics, prodrugs, receptors, binding agents, antibodies, small molecules or other drugs, or ribozymes) that alter (e.g., increase or decrease) the activity of the polypeptides described herein, or which otherwise interact with the polypeptides herein. For example, such agents can be agents which bind to polypeptides described herein (e.g., MAP3K9 binding agents); which have a stimulatory or inhibitory effect on, for example, activity of polypeptides of the invention; or which change (e.g., enhance or inhibit) the ability of the polypeptides of the invention to interact with MAP3K9 binding agents (e.g., receptors or other binding agents); or which alter posttranslational processing of the MAP3K9 polypeptide (e.g., agents that alter proteolytic processing to direct the polypeptide from where it is normally synthesized to another location in the cell, such as the cell surface; agents that alter proteolytic processing such that more polypeptide is released from the cell, etc.

In one aspect, the invention provides assays for screening candidate or test agents that bind to or modulate the activity of polypeptides described herein (or biologically active portion(s) thereof), as well as agents identifiable by the assays. Test agents can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam, K. S., Anticancer Drug Des. 12:145 (1997)).

In one aspect, to identify agents which alter the activity of a MAP3K9 polypeptide, a cell, cell lysate, or solution containing or expressing a MAP3K9 polypeptide, or another splicing variant encoded by a MAP3K9 gene or a fragment or derivative thereof (as described above), can be contacted with an agent to be tested; alternatively, the polypeptide can be contacted directly with the agent to be tested. The level (amount) of MAP3K9 activity is assessed (e.g., the level (amount) of MAP3K9 activity is measured, either directly or indirectly), and is compared with the level of activity in a control (i.e., the level of activity of the MAP3K9 polypeptide or active fragment or derivative thereof in the absence of the agent to be tested). If the level of the activity in the presence of the agent differs, by an amount that is statistically significant, from the level of the activity in the absence of the agent, then the agent is an agent that alters the activity of a MAP3K9 polypeptide. An increase in the level of MAP3K9 activity relative to a control sample indicates that the agent is an agent that enhances (is an agonist of) MAP3K9 activity. Similarly, a decrease in the level of MAP3K9 activity relative to a control indicates that the agent is an agent that inhibits (is an antagonist of) MAP3K9 activity. In another aspect, the level of activity of a MAP3K9 polypeptide or derivative or fragment thereof in the presence of the agent to be tested, is compared with a control level that has previously been established. A level of the activity in the presence of the agent that differs from the control level by an amount that is statistically significant indicates that the agent alters MAP3K9 activity.

The present invention also relates to an assay for identifying agents which alter the expression of a MAP3K9 nucleic acid (e.g., antisense nucleic acids, fusion proteins, polypeptides, peptidomimetics, prodrugs, receptors, binding agents, antibodies, small molecules or other drugs, or ribozymes) which alter (e.g., increase or decrease) expression (e.g., transcription or translation) of the gene or which otherwise interact with the nucleic acids described herein, as well as agents identifiable by the assays. For example, a solution containing a nucleic acid encoding a MAP3K9 polypeptide (e.g., a MAP3K9 gene or nucleic acid) can be contacted with an agent to be tested. The solution can comprise, for example, cells containing the nucleic acid or cell lysate containing the nucleic acid; alternatively, the solution can be another solution that comprises elements necessary for transcription/translation of the nucleic acid. Cells not suspended in solution can also be employed, if desired. The level and/or pattern of MAP3K9 expression (e.g., the level and/or pattern of mRNA or of protein expressed, such as the level and/or pattern of different splicing variants) is assessed, and is compared with the level and/or pattern of expression in a control (i.e., the level and/or pattern of the MAP3K9 expression in the absence of the agent to be tested). If the level and/or pattern in the presence of the agent differs by an amount or in a manner that is statistically significant, from the level and/or pattern in the absence of the agent, then the agent is an agent that alters the expression of a asthma gene. Enhancement of MAP3K9 expression indicates that the agent is an agonist of MAP3K9 activity. Similarly, inhibition of MAP3K9 expression indicates that the agent is an antagonist of MAP3K9 activity. In another aspect, the level and/or pattern of MAP3K9 polypeptide(s) (e.g., different splicing variants) in the presence of the agent to be tested, is compared with a control level and/or pattern that have previously been established. A level and/or pattern in the presence of the agent that differs from the control level and/or pattern by an amount or in a manner that is statistically significant indicates that the agent alters MAP3K9 expression.

In another aspect of the invention, agents which alter the expression of a MAP3K9 nucleic acid or which otherwise interact with the nucleic acids described herein, can be identified using a cell, cell lysate, or solution containing a nucleic acid encoding the promoter region of the MAP3K9 gene or nucleic acid operably linked to a reporter gene. After contact with an agent to be tested, the level of expression of the reporter gene (e.g., the level of mRNA or of protein expressed) is assessed, and is compared with the level of expression in a control (i.e., the level of the expression of the reporter gene in the absence of the agent to be tested). If the level in the presence of the agent differs, by an amount or in a manner that is statistically significant, from the level in the absence of the agent, then the agent is an agent that alters the expression of the MAP3K9, as indicated by its ability to alter expression of a gene that is operably linked to the MAP3K9 gene promoter. Enhancement of the expression of the reporter indicates that the agent is an agonist of MAP3K9 activity. Similarly, inhibition of the expression of the reporter indicates that the agent is an antagonist of MAP3K9 activity. In another aspect, the level of expression of the reporter in the presence of the agent to be tested is compared with a control level that has previously been established. A level in the presence of the agent that differs from the control level by an amount or in a manner that is statistically significant indicates that the agent alters expression.

Agents which alter the amounts of different splicing variants encoded by a MAP3K9 nucleic acid (e.g., an agent which enhances activity of a first splicing variant, and which inhibits activity of a second splicing variant), as well as agents which are agonists of activity of a first splicing variant and antagonists of activity of a second splicing variant, can easily be identified using these methods described above.

In other aspects of the invention, assays can be used to assess the impact of a test agent on the activity of a polypeptide in relation to a MAP3K9 binding agent. For example, a cell that expresses a compound that interacts with a MAP3K9 polypeptide (herein referred to as a “MAP3K9 binding agent”, which can be a polypeptide or other molecule that interacts with a MAP3K9 polypeptide, such as a receptor) is contacted with a MAP3K9 in the presence of a test agent, and the ability of the test agent to alter the interaction between the MAP3K9 and the MAP3K9 binding agent is determined. Alternatively, a cell lysate or a solution containing the MAP3K9 binding agent, can be used. An agent that binds to the MAP3K9 or the MAP3K9 binding agent can alter the interaction by interfering with, or enhancing the ability of the MAP3K9 to bind to, associate with, or otherwise interact with the MAP3K9 binding agent. Determining the ability of the test agent to bind to a MAP3K9 nucleic acid or a MAP3K9 binding agent can be accomplished, for example, by coupling the test agent with a radioisotope or enzymatic label such that binding of the test agent to the polypeptide can be determined by detecting the labeled with ¹²⁵I, ³⁵S, ¹⁴C or ³H, either directly or indirectly, and the radioisotope detected by direct counting of radioemmission or by scintillation counting. Alternatively, test agents can be enzymatically labeled with, for example, horseradish peroxidase, alkaline phosphatase, or luciferase, and the enzymatic label detected by determination of conversion of an appropriate substrate to product. It is also within the scope of this invention to determine the ability of a test agent to interact with the polypeptide without the labeling of any of the interactants. For example, a microphysiometer can be used to detect the interaction of a test agent with a MAP3K9 polypeptide or a MAP3K9 binding agent without the labeling of either the test agent, MAP3K9 polypeptide, or the MAP3K9 binding agent. McConnell, H. M. et al., Science 257:1906-1912 (1992). As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between ligand and polypeptide.

Thus, these receptors can be used to screen for compounds that are agonists or antagonists, for use in treating a susceptibility to a disease or condition associated with a MAP3K9 gene or nucleic acid, or for studying a susceptibility to a disease or condition associated with a MAP3K9 (e.g., asthma). Drugs could be designed to regulate MAP3K9 activation that in turn can be used to regulate signaling pathways and transcription events of genes downstream.

In another aspect of the invention, assays can be used to identify polypeptides that interact with one or more MAP3K9 polypeptides, as described herein. For example, a yeast two-hybrid system such as that described by Fields and Song (Fields, S. and Song, O., Nature 340:245-246 (1989)) can be used to identify polypeptides that interact with one or more MAP3K9 polypeptides. In such a yeast two-hybrid system, vectors are constructed based on the flexibility of a transcription factor that has two functional domains (a DNA binding domain and a transcription activation domain). If the two domains are separated but fused to two different proteins that interact with one another, transcriptional activation can be achieved, and transcription of specific markers (e.g., nutritional markers such as His and Ade, or color markers such as lacZ) can be used to identify the presence of interaction and transcriptional activation. For example, in the methods of the invention, a first vector is used which includes a nucleic acid encoding a DNA binding domain and also a MAP3K9 polypeptide, splicing variant, or fragment or derivative thereof, and a second vector is used which includes a nucleic acid encoding a transcription activation domain and also a nucleic acid encoding a polypeptide which potentially may interact with the MAP3K9 polypeptide, splicing variant, or fragment or derivative thereof (e.g., a MAP3K9 polypeptide binding agent or receptor). Incubation of yeast containing the first vector and the second vector under appropriate conditions (e.g., mating conditions such as used in the Matchmaker™ system from Clontech (Palo Alto, Calif., USA)) allows identification of colonies that express the markers of interest. These colonies can be examined to identify the polypeptide(s) that interact with the MAP3K9 polypeptide or fragment or derivative thereof. Such polypeptides may be useful as agents that alter the activity of expression of a MAP3K9 polypeptide, as described above.

In more than one aspect of the above assay methods of the present invention, it may be desirable to immobilize either the MAP3K9 gene or nucleic acid, the MAP3K9 polypeptide, the MAP3K9 binding agent, or other components of the assay on a solid support, in order to facilitate separation of complexed from uncomplexed forms of one or both of the polypeptides, as well as to accommodate automation of the assay. Binding of a test agent to the polypeptide, or interaction of the polypeptide with a binding agent in the presence and absence of a test agent, can be accomplished in any vessel suitable for containing the reactants. Examples of such vessels include microtitre plates, test tubes, and micro-centrifuge tubes. In one aspect, a fusion protein (e.g., a glutathione-S-transferase fusion protein) can be provided which adds a domain that allows a MAP3K9 nucleic acid, MAP3K9 polypeptide, or a MAP3K9 binding agent to be bound to a matrix or other solid support.

In another aspect, modulators of expression of nucleic acid molecules of the invention are identified in a method wherein a cell, cell lysate, or solution containing a MAP3K9 nucleic acid is contacted with a test agent and the expression of appropriate mRNA or polypeptide (e.g., splicing variant(s)) in the cell, cell lysate, or solution, is determined. The level of expression of appropriate mRNA or polypeptide(s) in the presence of the test agent is compared to the level of expression of mRNA or polypeptide(s) in the absence of the test agent. The test agent can then be identified as a modulator of expression based on this comparison. For example, when expression of mRNA or polypeptide is greater (statistically significantly greater) in the presence of the test agent than in its absence, the test agent is identified as a stimulator or enhancer of the mRNA or polypeptide expression. Alternatively, when expression of the mRNA or polypeptide is less (statistically significantly less) in the presence of the test agent than in its absence, the test agent is identified as an inhibitor of the mRNA or polypeptide expression. The level of mRNA or polypeptide expression in the cells can be determined by methods described herein for detecting mRNA or polypeptide.

This invention further pertains to novel agents identified by the above-described screening assays. Accordingly, it is within the scope of this invention to further use an agent identified as described herein in an appropriate animal model. For example, an agent identified as described herein (e.g., a test agent that is a modulating agent, an antisense nucleic acid molecule, a specific antibody, or a polypeptide-binding agent) can be used in an animal model to determine the efficacy, toxicity, or side effects of treatment with such an agent. Alternatively, an agent identified as described herein can be used in an animal model to determine the mechanism of action of such an agent.

Furthermore, this invention pertains to uses of novel agents identified by the above-described screening assays for treatments as described herein. In addition, an agent identified as described herein can be used to alter activity of a polypeptide encoded by a MAP3K9 nucleic acid, or to alter expression of a MAP3K9 nucleic acid, by contacting the polypeptide or the nucleic acid (or contacting a cell comprising the polypeptide or the nucleic acid) with the agent identified as described herein.

Nucleic Acid Pharmaceutical Compositions

The present invention also pertains to pharmaceutical compositions comprising nucleic acids described herein, particularly nucleotides encoding the polypeptides described herein (e.g., a MAP3K9 polypeptide); comprising polypeptides described herein and/or comprising other splicing variants encoded by a MAP3K9 nucleic acid; and/or an agent that alters (e.g., enhances or inhibits) MAP3K9 nucleic acid expression or MAP3K9 polypeptide activity as described herein. For instance, a polypeptide, protein (e.g., a MAP3K9 nucleic acid receptor), an agent that alters MAP3K9 nucleic acid expression, or a MAP3K9 binding agent or binding partner, fragment, fusion protein or pro-drug thereof, or a nucleotide or nucleic acid construct (vector) comprising a nucleotide of the present invention, or an agent that alters MAP3K9 polypeptide activity, can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms, and should be decided according to the judgment of a practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

The present invention is now illustrated by the following Exemplification, which is not intended to be limiting in any way. All references cited herein are incorporated by reference in their entirety.

### EXAMPLES

### Example 1

Patient Population

The original patient list contained the names of over 7,000 patients who attended the private clinics or outpatient clinics of allergists practicing at the Allergy/Pulmonary Divisions of the National University Hospital of Iceland during the years 1977 to 2001 (ECRHSG; 1997). For this study, patients were selected with physician-diagnosed asthma who were being treated with asthma drugs and who were related to at least one other patient within and including 6 meiotic events (6 meiotic events separate 2^{nd} cousins) as revealed by a computerized genealogy database. Ages ranged from 12-70 years (mean 39.3 yrs) and 62% were females. Information regarding the age at diagnosis, medications, hospital admissions, and family history of atopy and asthma were gathered. The diagnosis of asthma and atopy were clinically re-confirmed in the study by a new physical examination, measurements of skin test reactivity to 12 aeroallergens (including birch, grass, Rumex crispus, cat, dog, horse, Cladosporium, Mucor, Alternaria, Dermatophagoides pteronyssinus, D. farinae, and Lepidoglyphus destructor), total IgE levels, and pulmonary function tests (PFT). Unless a baseline forced expiratory volume in 1 s (FEV1) was ≦70% of predicted value (based on sex, height, and race), a methacholine (MCh)-challenge test was performed. The phenotype assessments, PFTs, and methacholine tests were performed according to ATS guidelines (Cockcroft, et al., 1977; Palmquist, et al., 1988). Patients were considered as being atopic if their skin prick test reaction was positive (i.e., ≧3 mm or ≧50% of the histamine positive control response). The diagnosis of asthma in Iceland is based on the diagnostic criteria outlined by the NHLB and the American Thoracic Society (National Institutes of Health 1997; American Thoracic Society 1995) and includes any of the following measures:

Patient having recurrent symptoms of cough and wheezing for more than 2 years and demonstrating clinical response to bronchodilator therapy (as measured by >15% increase in FEV1 following bronchodilator treatment).

Patient having reduced FEV1 (FEV1<80%) at baseline prior to bronchodilator therapy and showing >15% improvement in FEV1 following bronchodilator therapy.

Patient having recurrent symptoms of cough and wheezing and on methacholine challenge test, performed in accordance to ATS guidelines (American Thoracic Society 1995), has >20% drop in FEV1 at methacholine concentrations ≦8 mg/L.

Severity of asthma was determined by the combination of signs and symptoms, PFTs, MCh values, and requirements for therapy. On the basis of these criteria, approximately 90% of the patients were scored as having mild to moderate asthma and 10% with severe asthma. FEV1>80% predicted was considered normal. Before blood samples were obtained, all patients included in the study were re-examined by the same two allergists who confirmed the asthma phenotype and severity level and supervised the measurements of total IgE levels, spirometry and skin testing prior to obtaining blood samples. In this study, a reduction in FEV1 of 20% or more at a MCh concentration of 8 mg/L or less is considered positive challenge test.

The patient participation rate for the study exceeded 90%. All patients signed informed consent, donated blood samples, and completed a detailed medical questionnaire and all tests necessary for proper phenotyping. The study was approved by the Icelandic Data Protection Commission and the National Bioethics Committee. Personal identities of the patients and their family members were subsequently encrypted by the Data Protection Commission of Iceland (Gulcher et al., 2000). All blood and DNA samples were also coded in the same way. All participants were asked, by questionnaire, whether they had been diagnosed with asthma and/or atopy and whether they were receiving drugs to treat if they were being treated. The names of the drugs were recorded and confirmed to be anti-asthma and/or allergy medications. Blood was also collected from close relatives of the index cases to increase the information available for the linkage analysis. All participants had their lung function measured at the time the blood was drawn for the study.

Pedigrees

deCODE has built a computerized genealogy database with over 650,000 names that includes all 285,000 living Icelanders and most of their ancestors (Gulcher and Stefansson 1998). The database has a connectivity of over 95% in the 20^{th} century and 86% in the 19^{th} century. Its maternal connections are 99.3% accurate as measured by mitochondrial polymorphisms of maternally linked individuals (Helgason et al., 2000). The genealogy database was used to cluster the patients in pedigrees. The genealogy database is reversibly encrypted by the Data Protection Commission of Iceland before it is used in our laboratory (Gulcher and Stefansson 1998). Recursive algorithms are used with the encrypted personal identifiers to find all ancestors in the database who are related to any member on the patient list within a given number of generations back. The cluster function then identifies ancestors who are common to any two or more members of the patient list.

Genotyping

Five hundred ninety-six patients were genotyped with asthma belonging to 175 families, in which each patient was related to at least one other patient within and including 6 meiotic events. DNA samples from all 596 patients and 538 relatives were successfully genotyped using 976 specific fluorescently labelled primers with an initial average spacing of 3-4 cM genome-wide. A microsatellite screening set was developed based in part on the ABI Linkage Marker (v2) screening set and the ABI Linkage Marker (v2) intercalating set in combination with over 500 custom-made markers. All markers were extensively tested for multiplex PCR reactions. The PCR amplifications were set up and pooled using Cyberlab robots. The reaction volume used was 5 μl and for each PCR reaction 20 ng of genomic DNA was amplified in the presence of 2 pmol of each primer, 0.25 U AmpliTaq Gold, 0.2 mmol/L dNTPs and 2.5 mmol/L MgCl₂. The PCR conditions used were 95° C. for 10 minutes, then 37 cycles of 15 s at 94° C., 30 s at 55° C. and 1 min at 72° C. The PCR products were supplemented with the internal size standard and the pools were separated and detected on Applied Biosystems model 3700 Sequencer using Genescan v3.0 peak calling software. Alleles were called automatically with the DAC program (Fjalldal et al., 2001), and the program, DecodeGT, was used to fractionate according to quality and edit the called genotypes (Palsson et al., 1999). In regions demonstrating linkage using the framework marker set, marker density was further increased (i.e., fine mapping of locus) by additional microsatellite markers to obtain coverage of 0.2 cM on the average in these regions.

Statistical Analyses

A genome-wide linkage scan was performed using a framework map of 976 microsatellite markers. The data was analyzed using the Allegro program (Gudbjartsson et al., 2000) and determined statistical significance by applying affecteds-only allele-sharing methods (not specifying any particular inheritance model). The Allegro program, a linkage program developed at deCODE genetics, calculates LOD scores based on multipoint calculations (Gudbjartsson et al., 2000; Kruglyak et al., 1996; Kong and Cox 1997) and is available for free for non-commercial use by sending e-mail to allegro@decode.is. The linkage analysis approach uses the Sₚₐᵢᵣₛ scoring function (Kruglyak et al., 1996; Whittemore and Halpern 1994), the exponential allele-sharing model (Kong and Cox 1997), and a family weighting scheme that is halfway, on the log scale, between weighting each affected pair equally and weighting each family equally. All genotyped individuals who are unaffected are treated as “unknown”. P values are calculated based on the large sample theory; Zlr=√(2 logₑ(10)LOD) is approximately distributed as a standard normal distribution under the null hypothesis of no linkage (Kong and Cox 1997) and the observed LOD score is compared to its complete data sampling distribution under the null hypothesis (Gudbjartsson et al., 2000); The information measure we use is part of the Allegro program output (Nicolae 1999) and closely related to a classical measure (Dempster et al., 1977). Information equals zero if the marker genotypes are completely uninformative and equals one if the genotypes determine the exact amount of allele sharing by descent among the affected relatives. The marker order and positions for the framework mapping set were obtained using a high-density genetic map developed at deCODE. Data from 146 Icelandic nuclear families (sibships with genotypes for two to seven siblings and both parents) providing 1257 meioses were analyzed to estimate the genetic distances. By comparison, distances in the Marshfield genetic map were estimated based on 188 meioses. Inter-marker distances in the peak region after enrichment with 4 markers were estimated using an adaptation of the EM algorithm (Dempster 1977) within Allegro.

Linkage Analysis

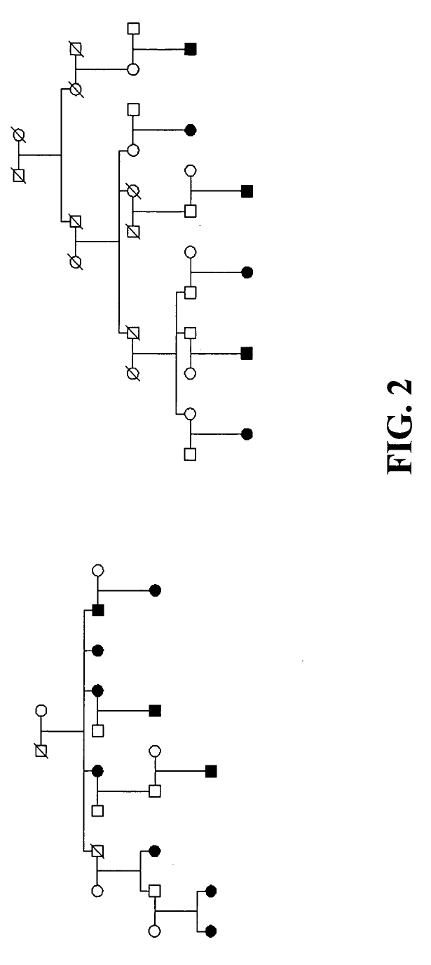

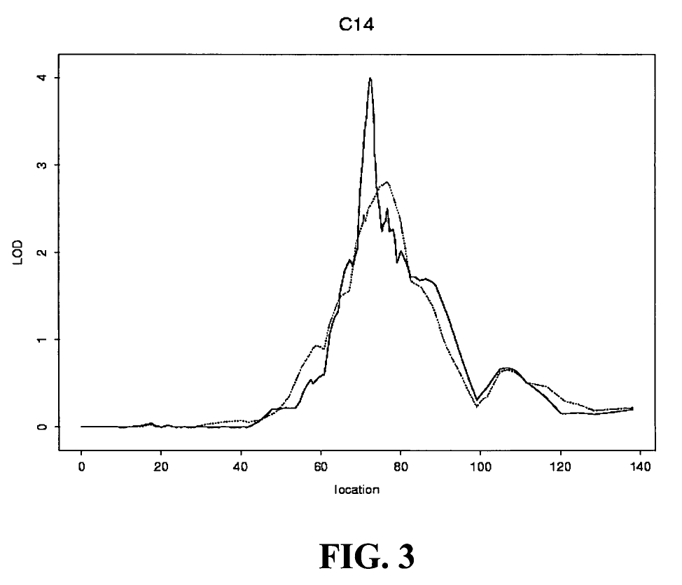

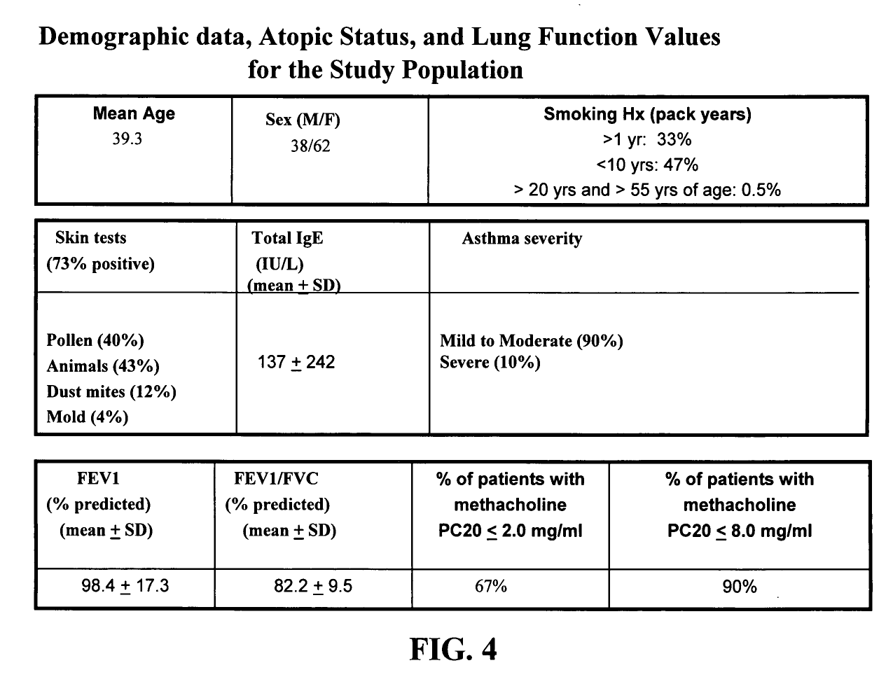

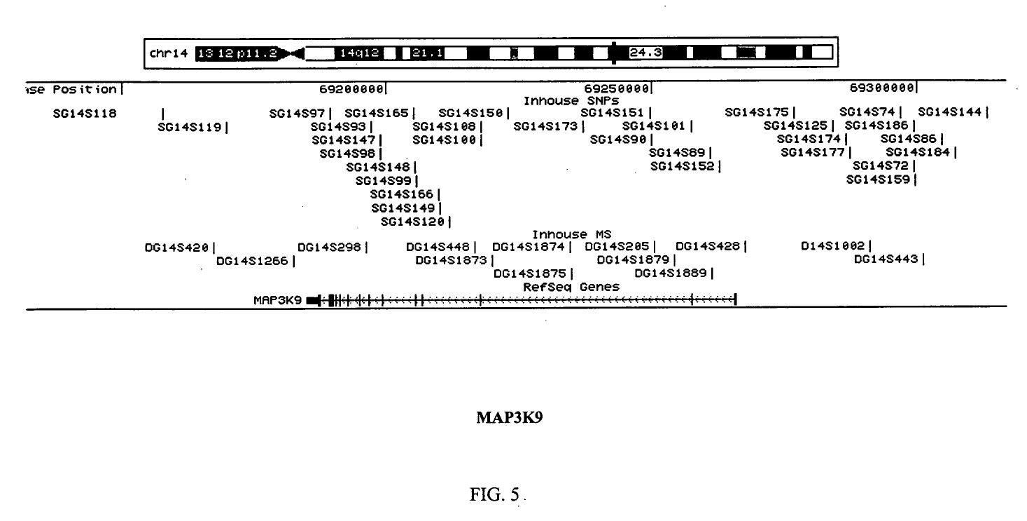

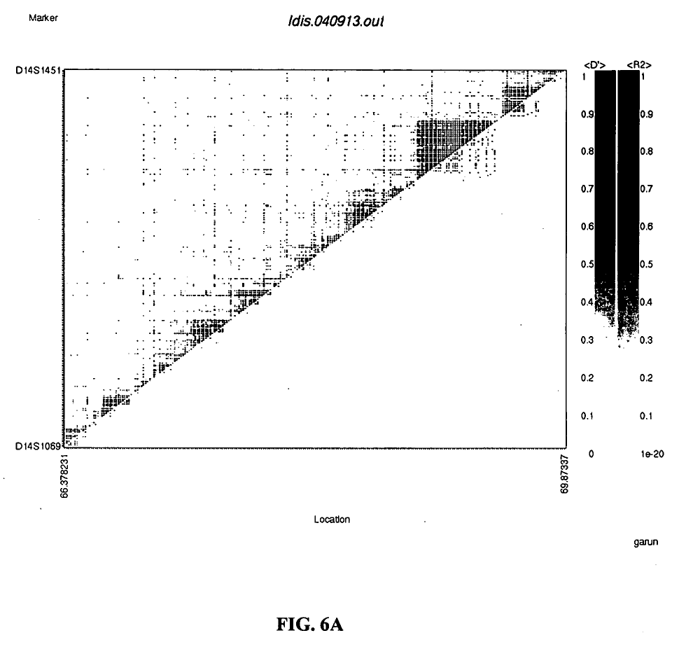

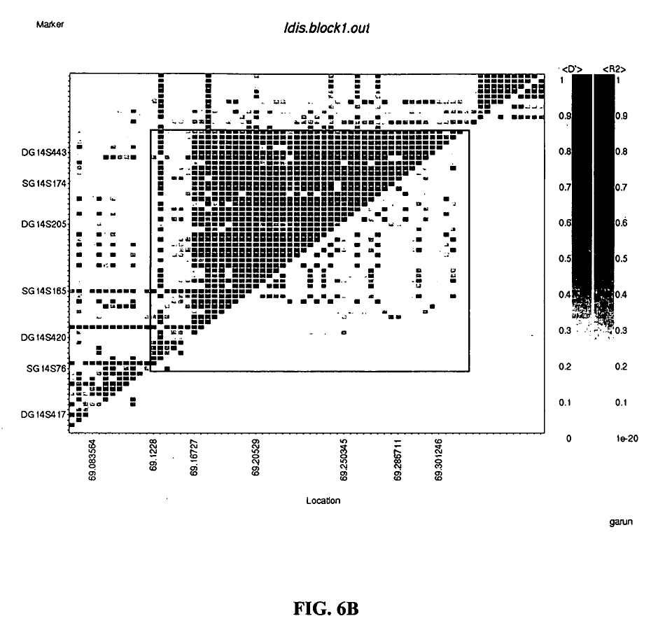

A list of over 7,000 patients from the National University Hospital of Iceland were cross-matched with the genealogy database. In the present study, patients with physician-diagnosed asthma who were related by 6 or fewer meiotic events to other patients with asthma (6 meiotic events separate 2^{nd} cousins) were included. These were 596 patients in 175 families with asthma. In the present study, over thirty families had at least 6 affected members each. Two of these families used in the analysis are displayed in FIG. 2. The patients' demographic data, geometric mean IgE values, lung function tests (including % predicted FEV1, and FEV1/FVC ratio), methacholine challenge test and skin test results to the most common aeroallergens in Iceland are shown in FIG. 4. Seventy-three percent of the patients were atopic as defined by a positive skin test reaction.

More than 400 of the study patients were tested for airway reactivity using MCh challenge. As shown in FIG. 4, although 67% of the patients tested had more than 20% drop in FEV1 at a MCh concentration of less than 2 mg/L, over 90% of the patients tested positive at a MCh concentration of 8 mg/L or less. This would be consistent with moderate to severe airway hyperresponsiveness in ⅔ of our asthma study population. The spirometric values reported in FIG. 4 are those obtained during the study at which time the majority of the patients had stable asthma and were on full therapy; however, all these patients have previous spirometric values with FEV1<80% predicted at one or more earlier time points in their medical charts (data not shown). Bronchodilator reversibility was tested in selective cases including those patients who had negative results of a MCh challenge and from whom clinician determined the test to be necessary to support the asthma phenotype.

Thirty-three percent of the patients gave a history of having smoked for more than 1 pack-year. Of those, 47 percent had smoked for fewer than 10 pack-years. The possibility that some or few of the study participants who are smokers had mild co-existing COPD cannot be excluded; however, only 0.5% of study the patients who were 55 years or older had smoked for more than 20 pack years (FIG. 4). In contrast, all study subjects have asthma as defined by the ATS criteria (American Thoracic Society; 1995); which is the phenotype used for this study thereby minimizing the potential of confounding effects from COPD.

Five hundred ninety-six patients and 538 of their unaffected (i.e., non-asthmatic) relatives were genotyped using 976 microsatellite markers in a genome-wide linkage scan. The disease status of relatives was obtained by a questionnaire. The data was analyzed and determined statistical significance by applying affecteds-only, allele-sharing methods (which do not specify any particular inheritance model) (Gulcher et al., 2001). Since the linkage scan was performed on affected status only, there is no possible confounding effect from the relatives even if an affected relative in the group was not identified. The genomic region that showed the most evidence for linkage to the asthma phenotype was chromosome 14q24 with a lod score of 2.66 (single test p=2.16×10⁻⁴). Given that the information content on identity by descent sharing in the region was less than 85% (i.e., 0.77), which is less than preferred, another 34 microsatellite markers under this peak were added to ensure that the results are a true reflection of the information contained in the material. This increased the information content at the peak to over 95% and produced a lod score of 4.00 (single test p=8.70×10⁻⁶). This locus is significant on a genome-wide basis, even allowing for the multiple marker testing, and it corresponds to a genome-wide adjusted P-value of less than 0.05 (Krugylak and Lander 1996). This locus was designated as asthma locus one (AS1). The locus peak is centered on markers D14S588 and D14S603, which are spaced 84 kb apart. The locus, defined by a drop of approximately 1.0 in the LOD score, is between markers D14S1069 and D14S289 centromeric, and telomeric respectively. The segment with a 1-LOD drop is around 3.9 centimorgans and is estimated to correspond to around 3.0 million bases. The genome scan shows for the first time significant linkage of asthma to markers on chromosome 14q24. It should be noted that a suggestive linkage to this locus has been previously demonstrated by investigators in the United Kingdom (Mansur 1999) and the United States (The Collaborative Study of the Genetics of Asthma 1997). Chromosome 14q24 contains many genes that could contribute to the susceptibility to asthma, including genes that encode for the EGF-response factor 1, phosphatidylinositol glycan class H, secreted modular calcium-binding protein 1, a disintegrin and metalloproteinase domain-20 and -21, and RNA polymerase II transcriptional regulation mediator, to name a few.

### Example 2

Assessment of MAP3K9 Expression in Human Airway Tissue:

Lung tissue from 2 asthma patients and 2 controls (all 4 are smokers who developed lung cancer and needed resection) were studied. Airway tissue from a non-cancerous (healthy) part of their small airways was isolated and examined for expression of MAP3K9 using RT-PCR. A 10 fold increased expression of the b-isoform of MAP3K9 was observed in these 2 patients (see FIG. 9) compared to controls.

### Example 3

Assessment of MAP3K9 Expression in Peripheral Blood Mononuclear (PBM) Cells:

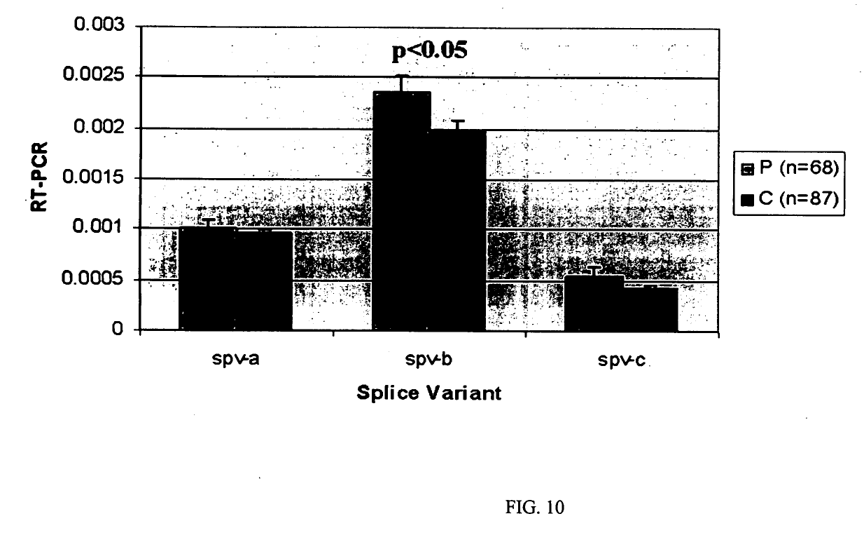

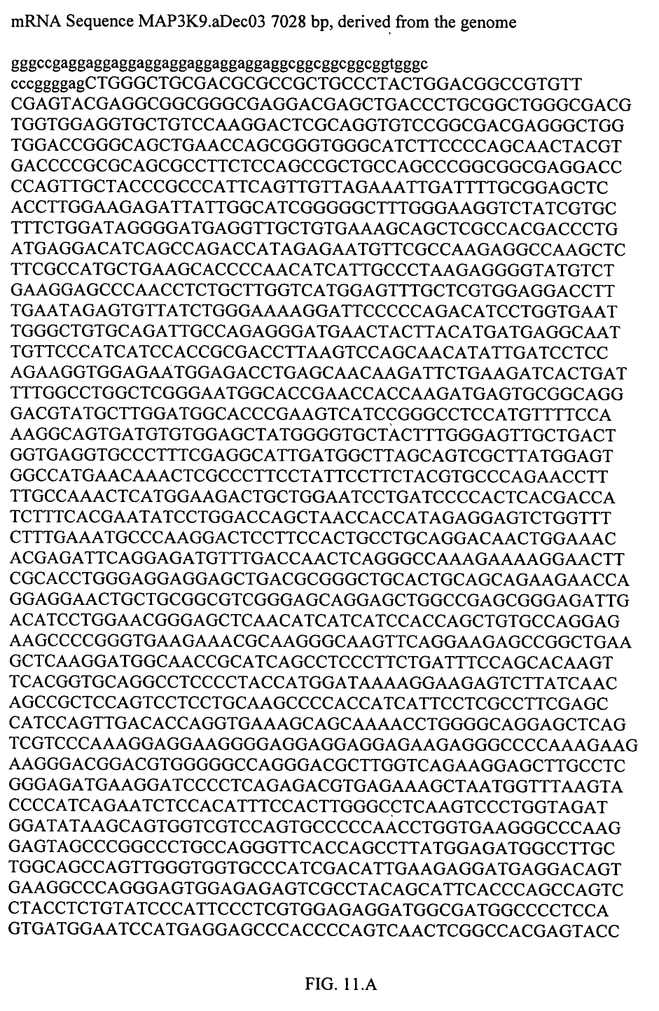

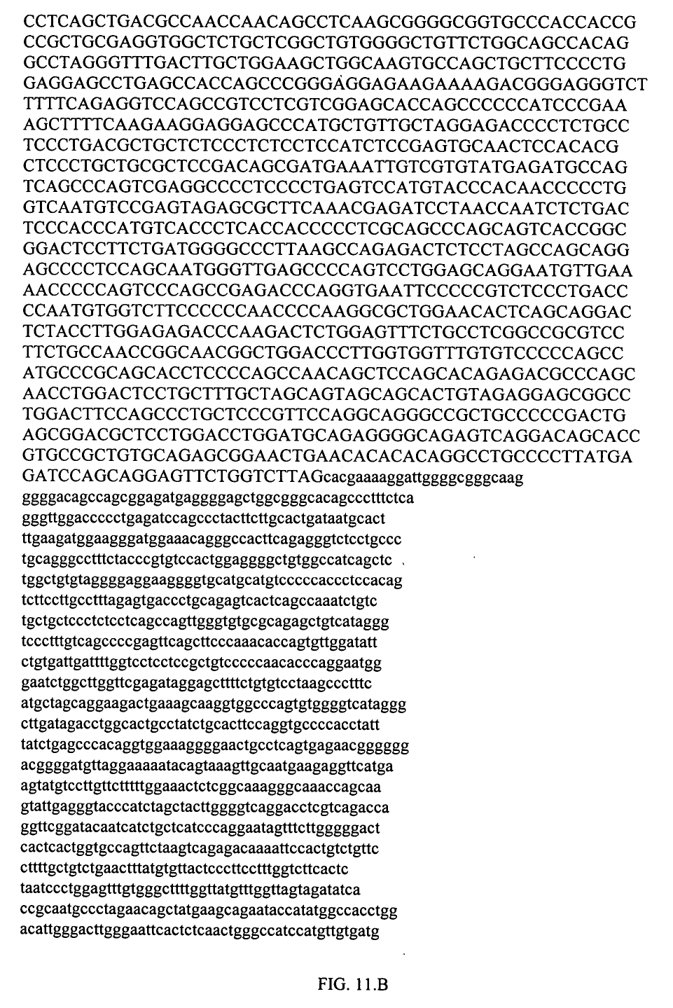

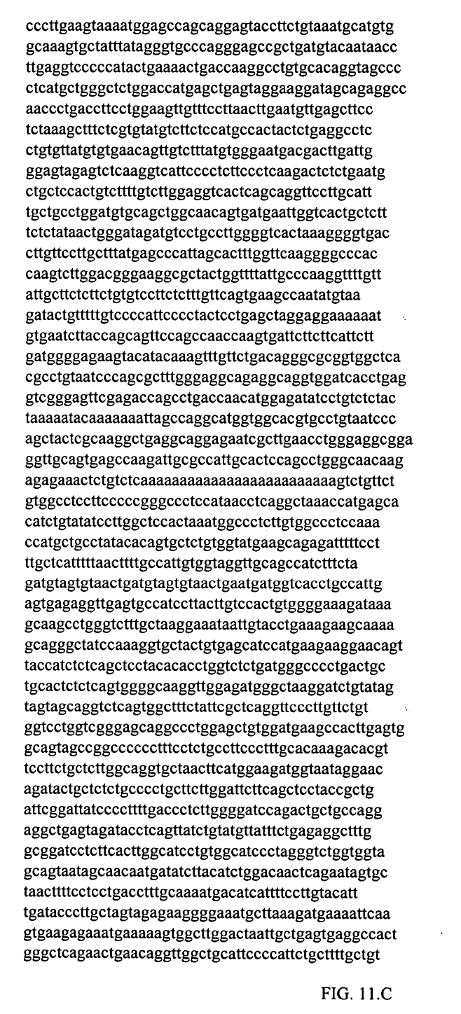

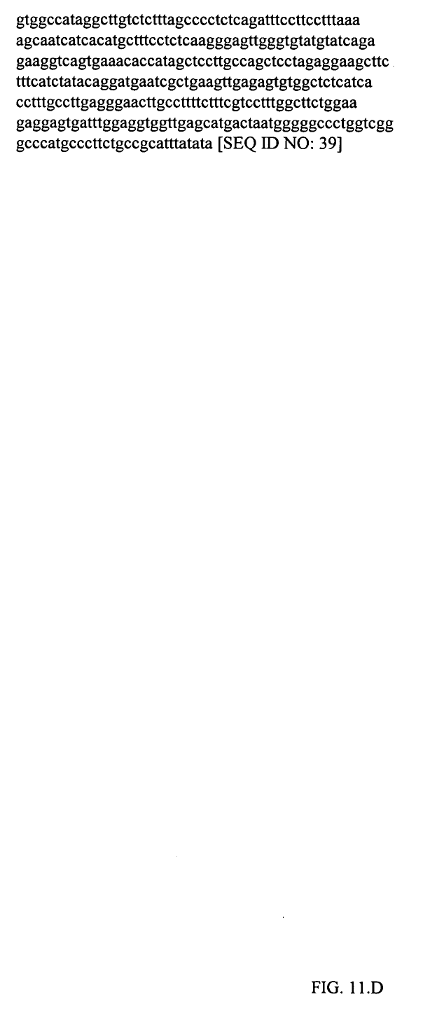

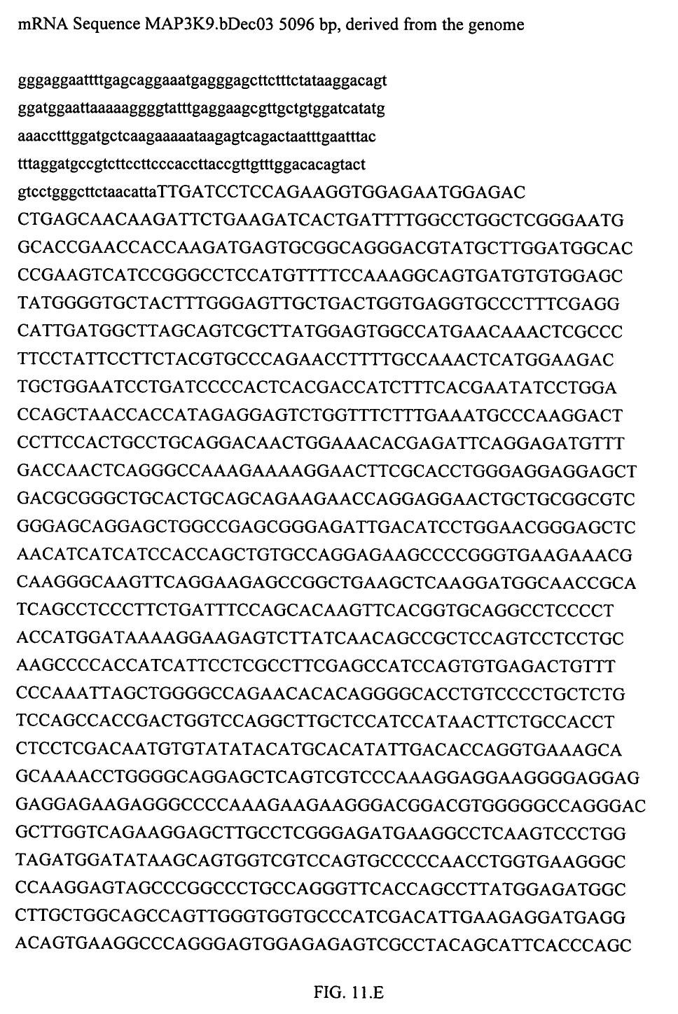

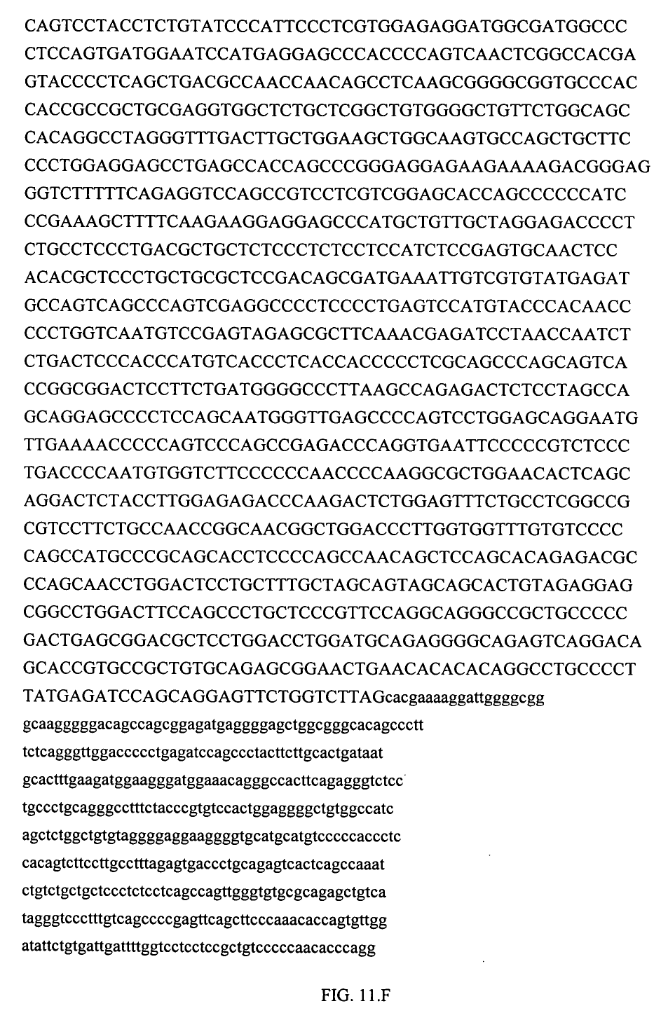

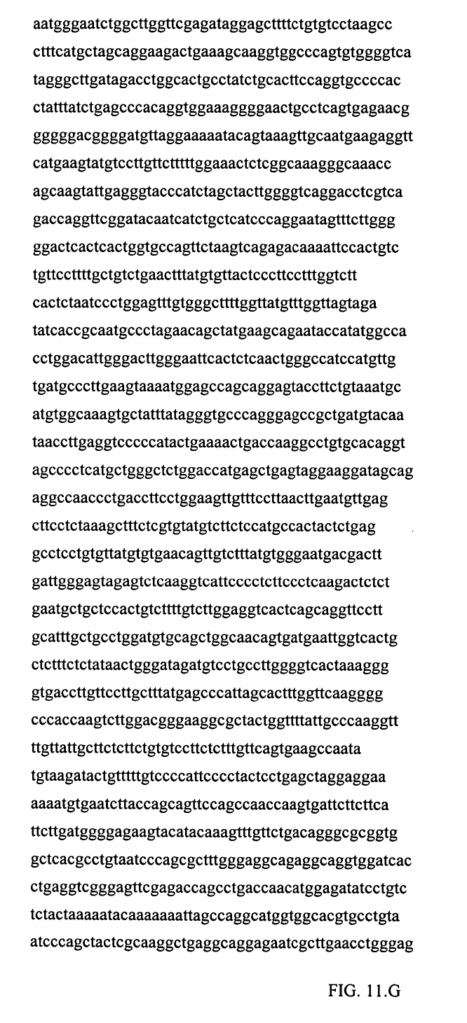

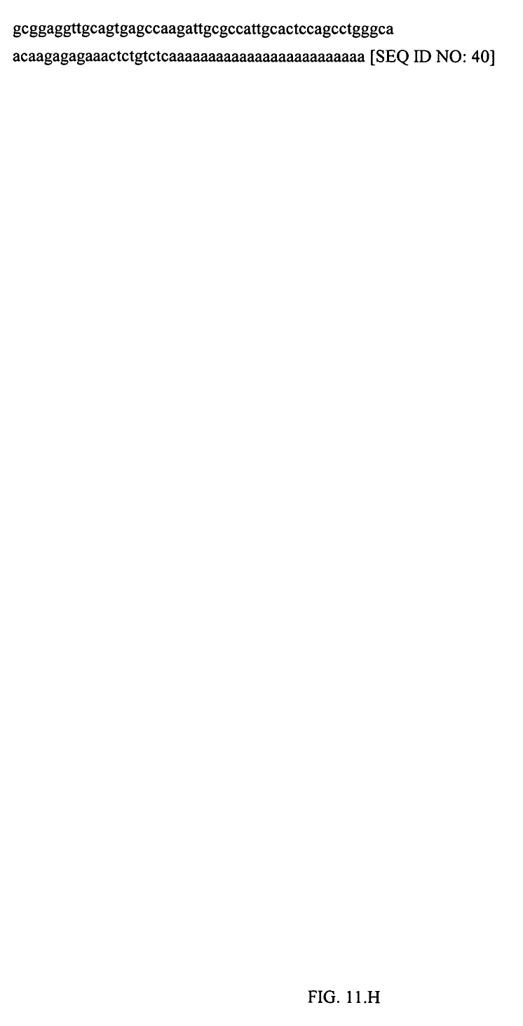

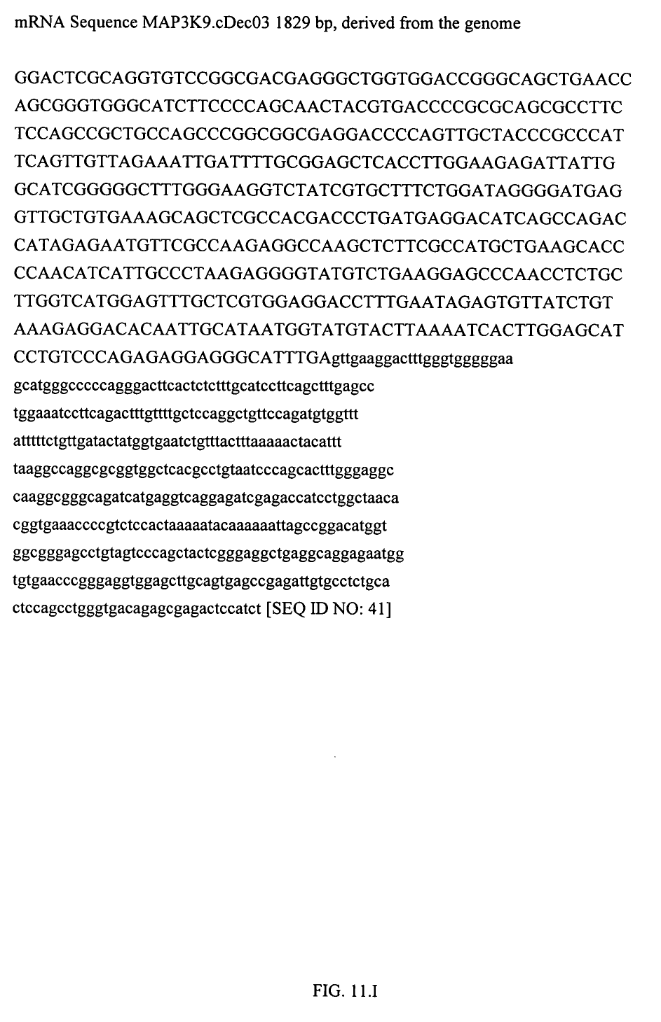

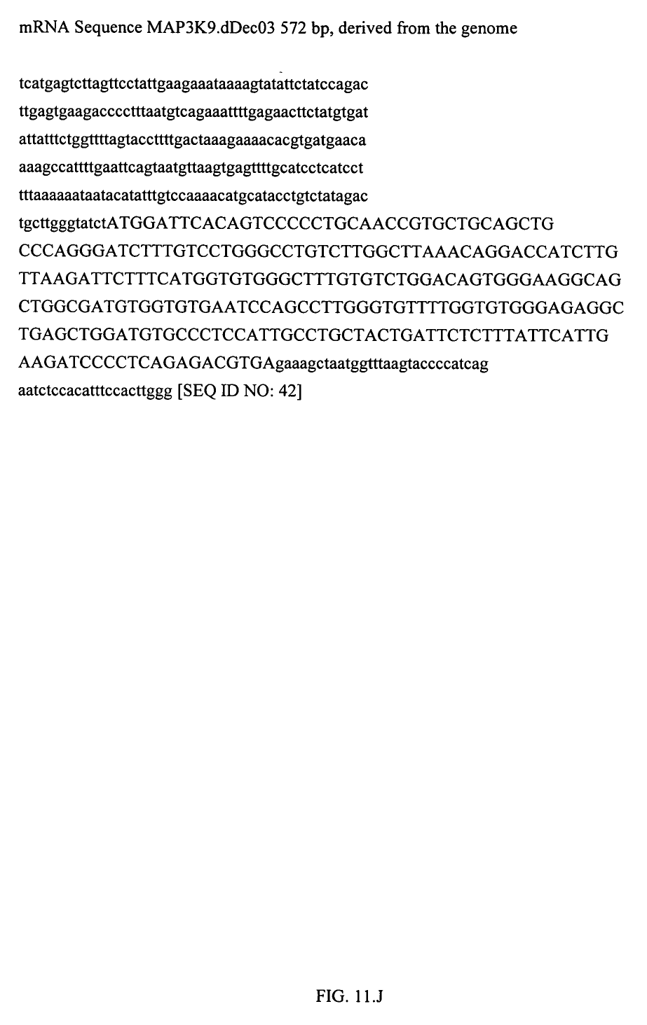

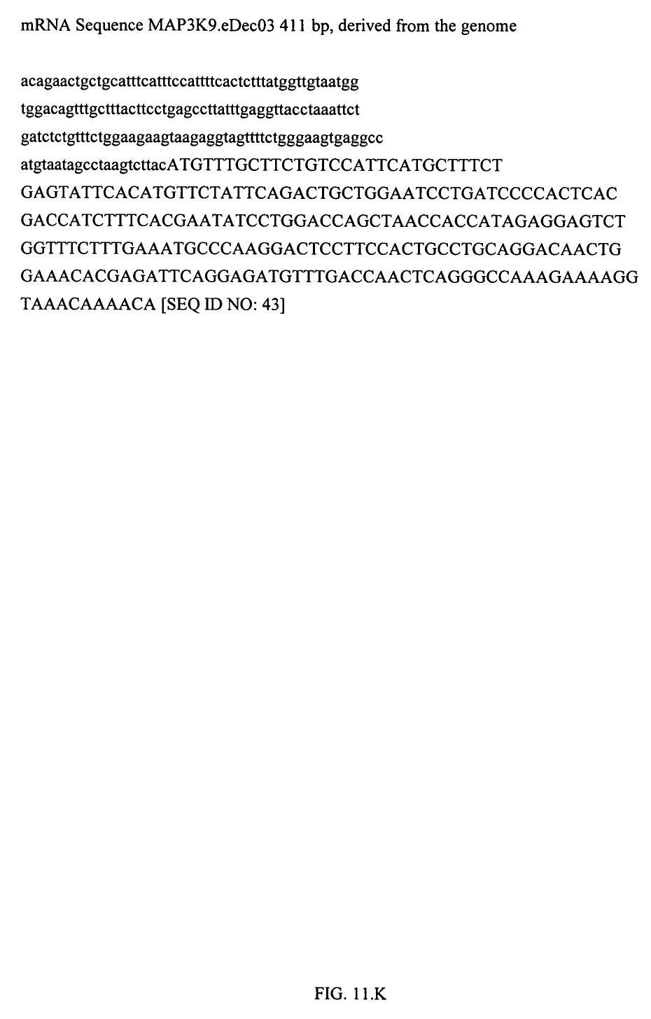

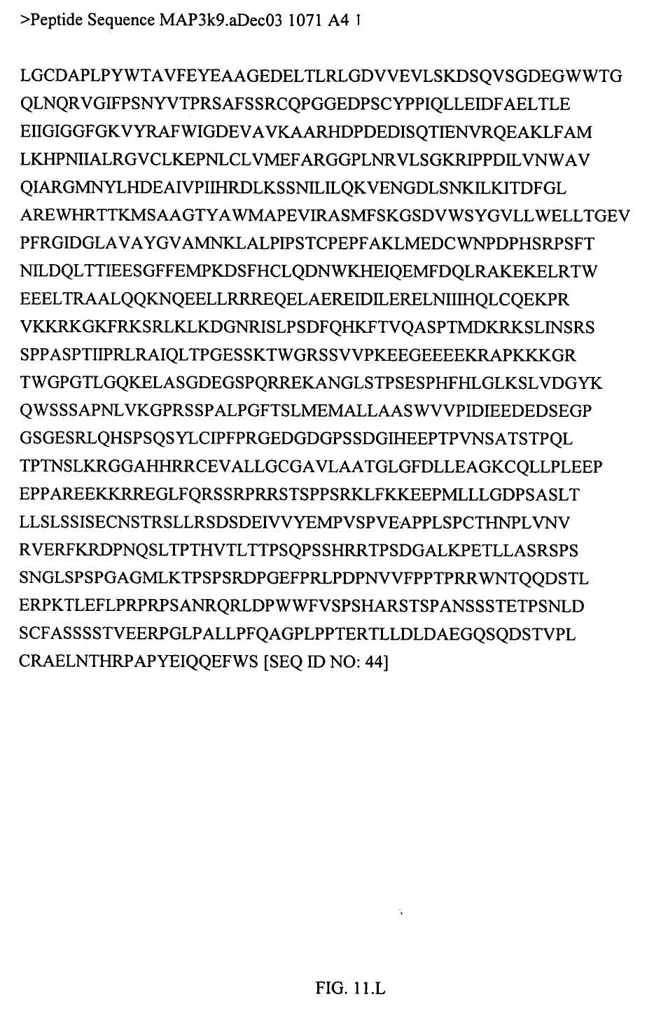

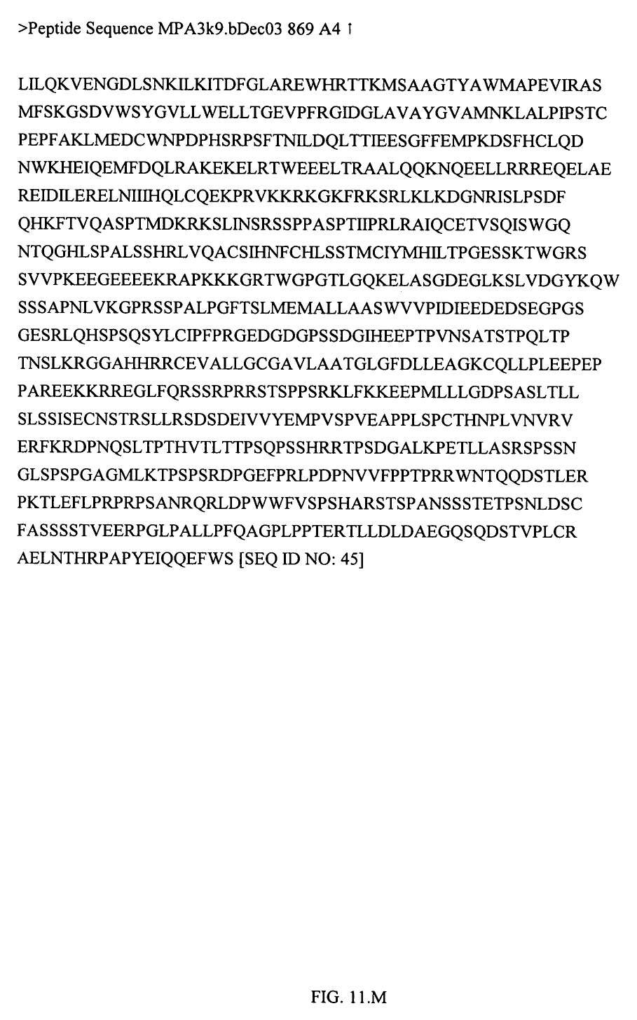

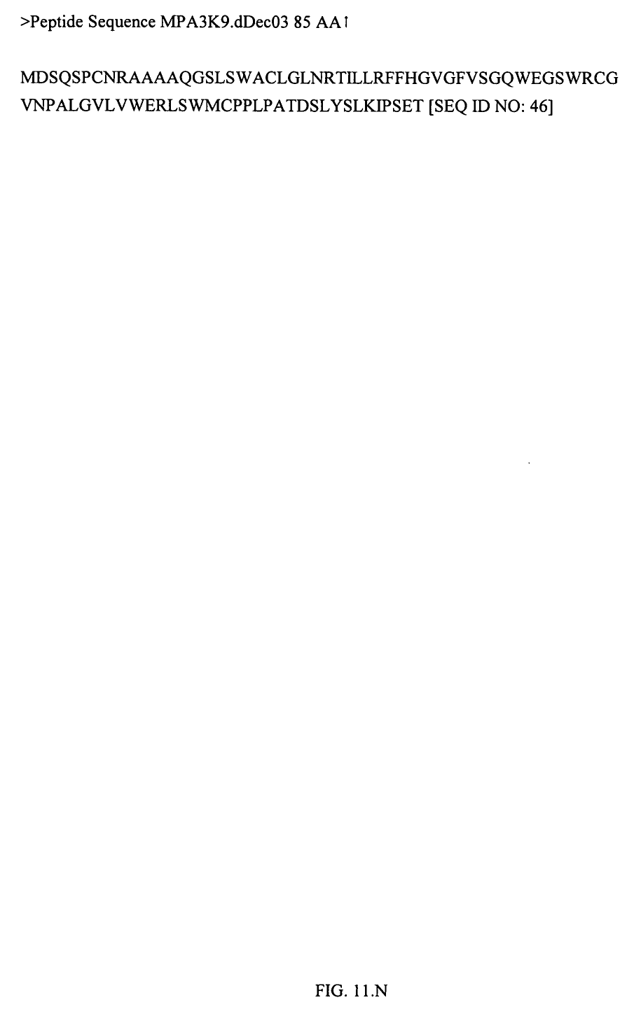

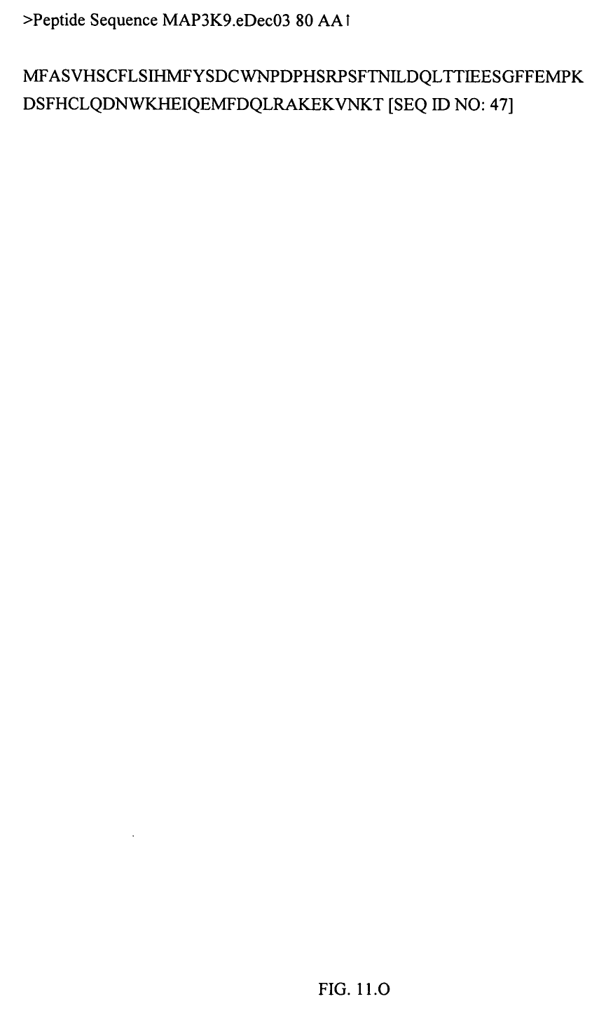

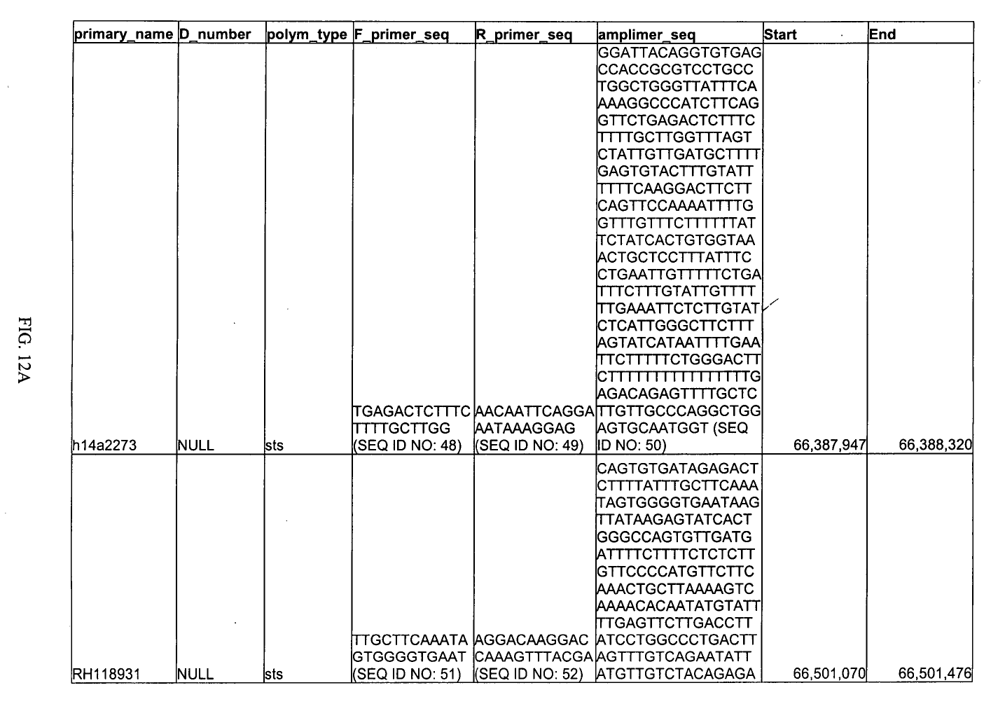

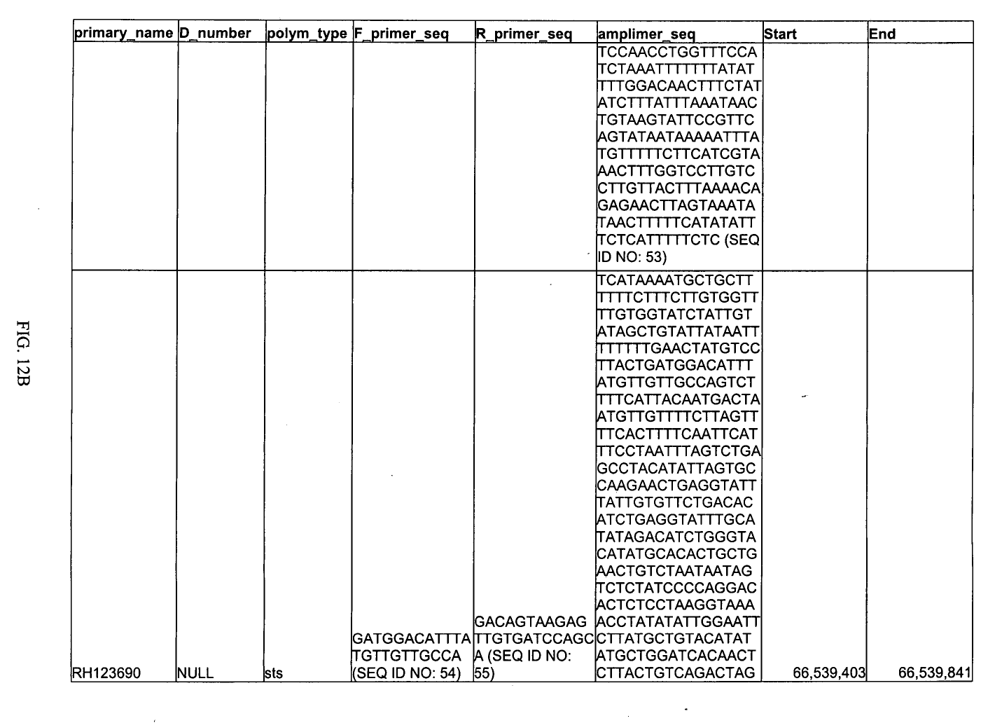

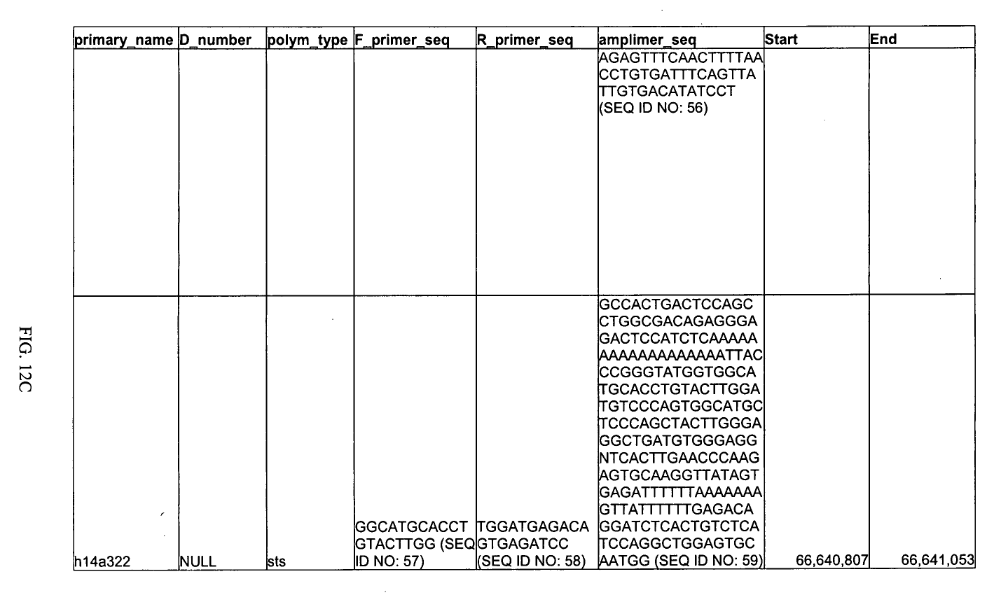

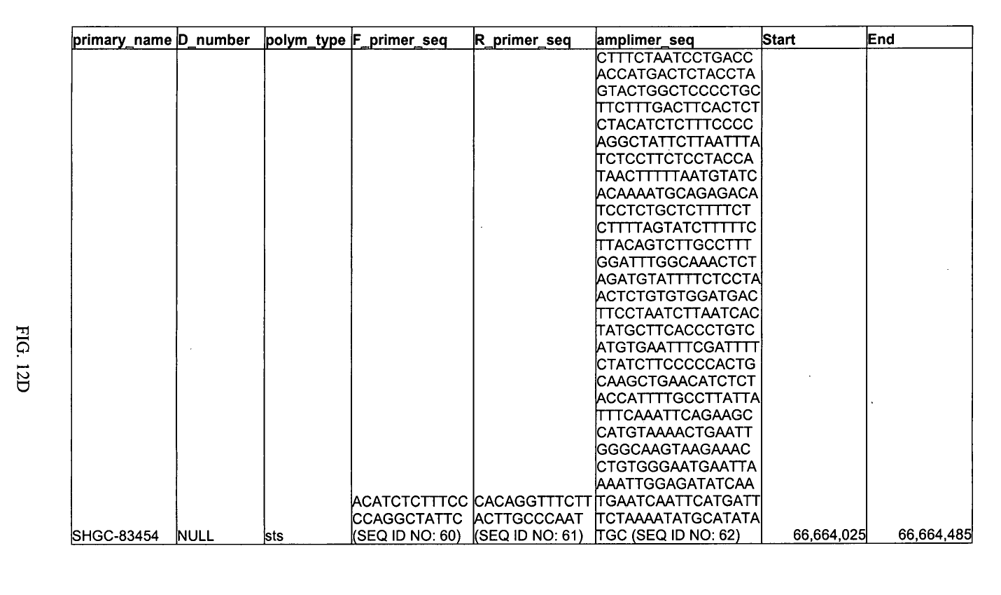

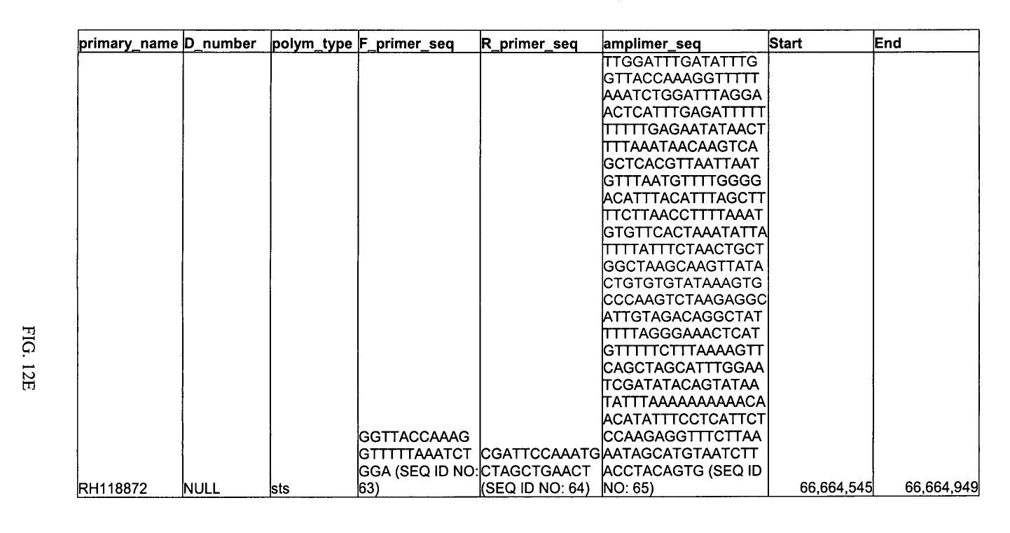

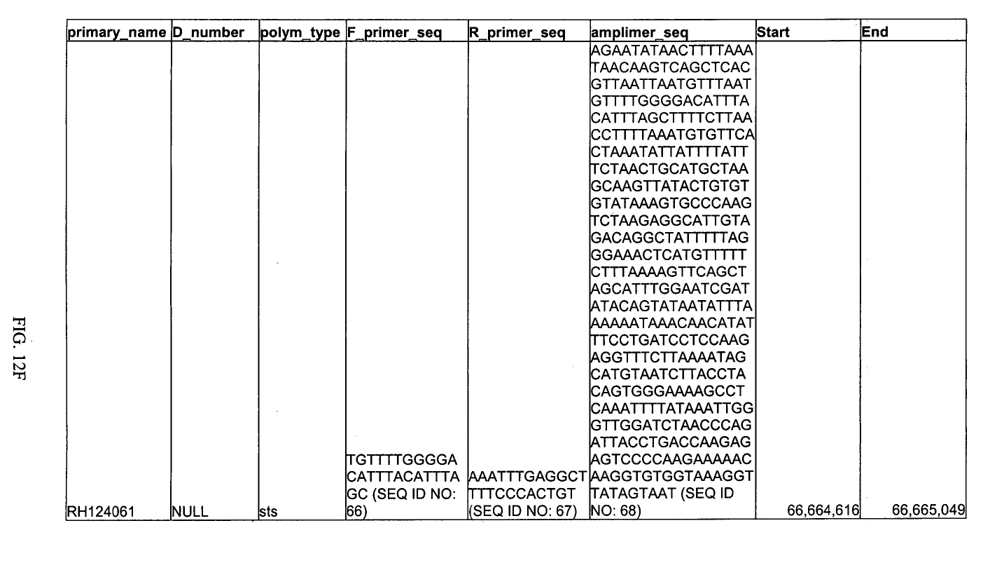

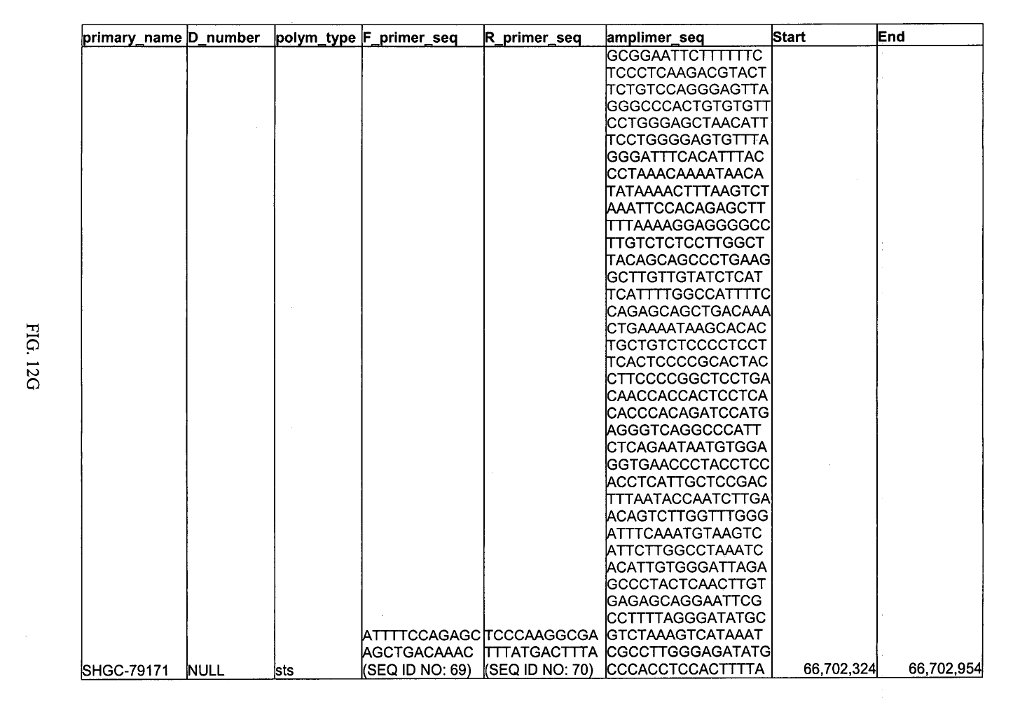

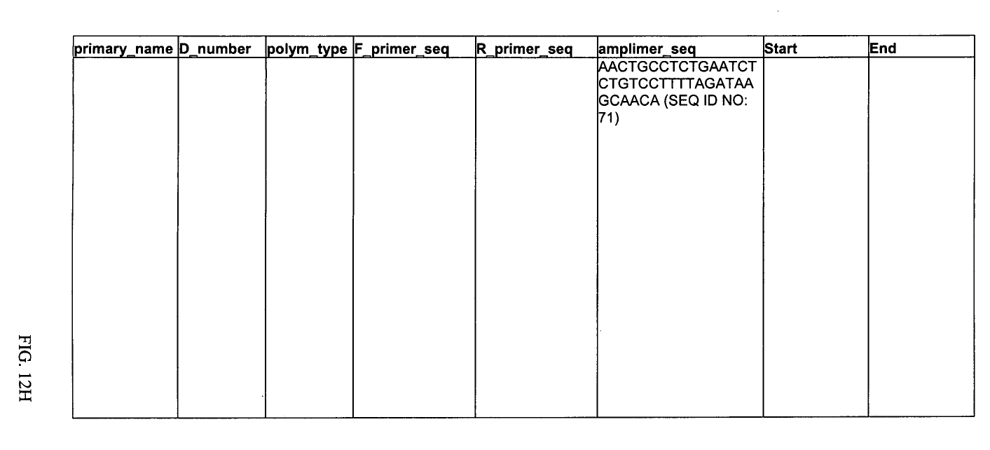

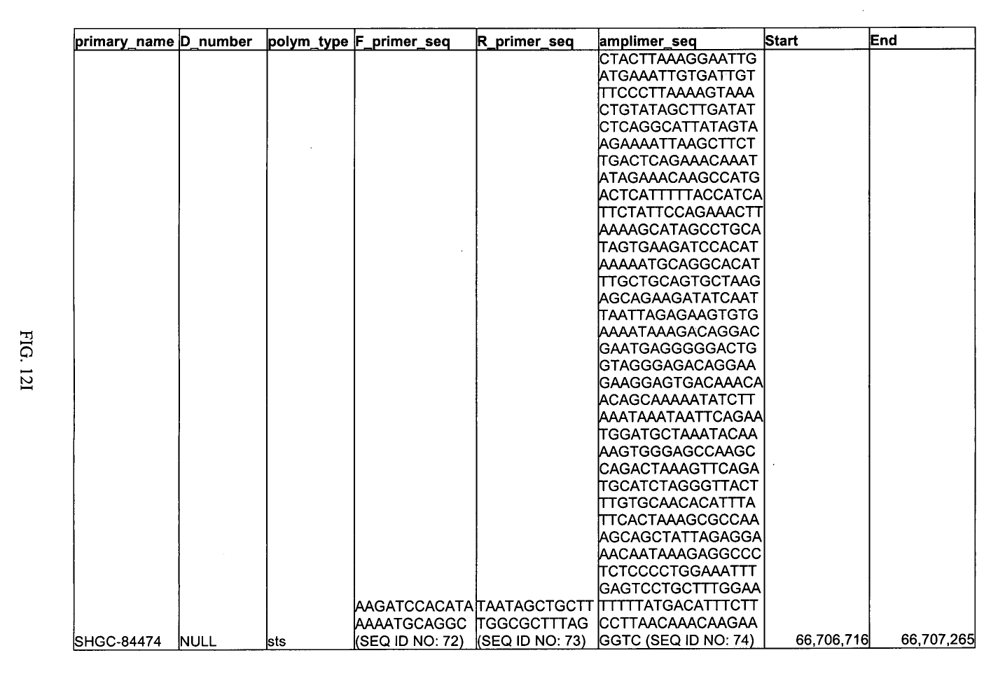

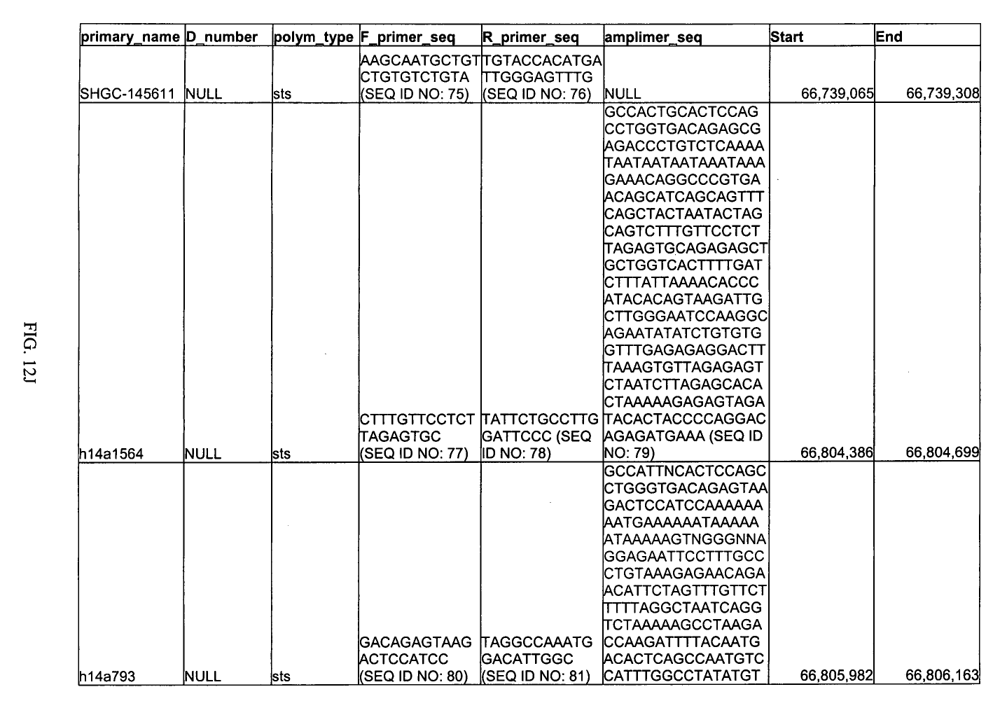

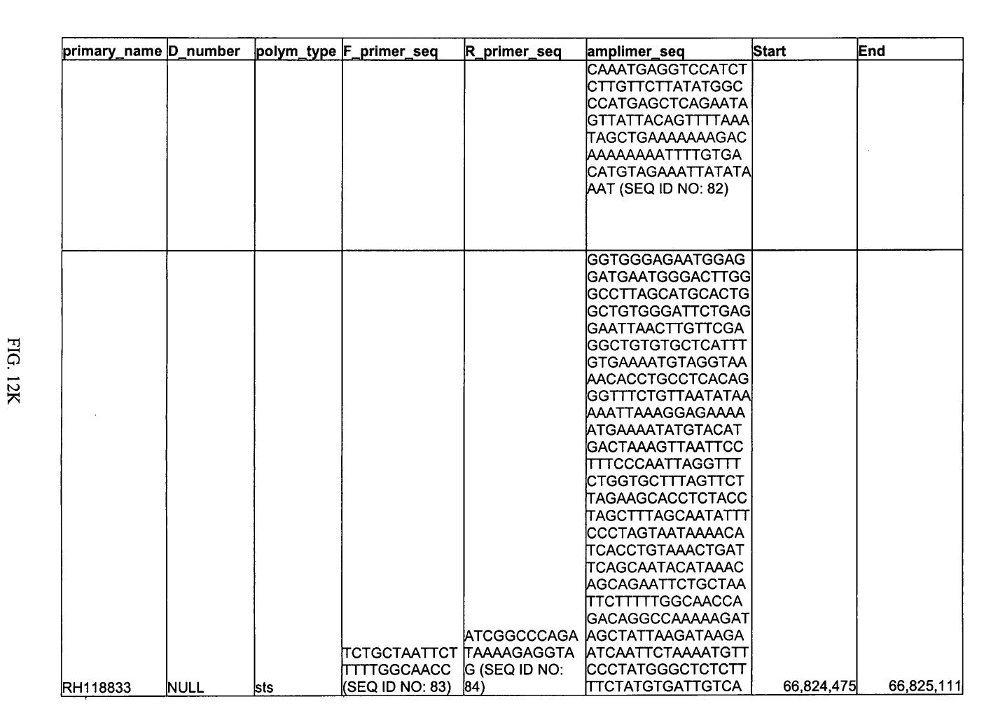

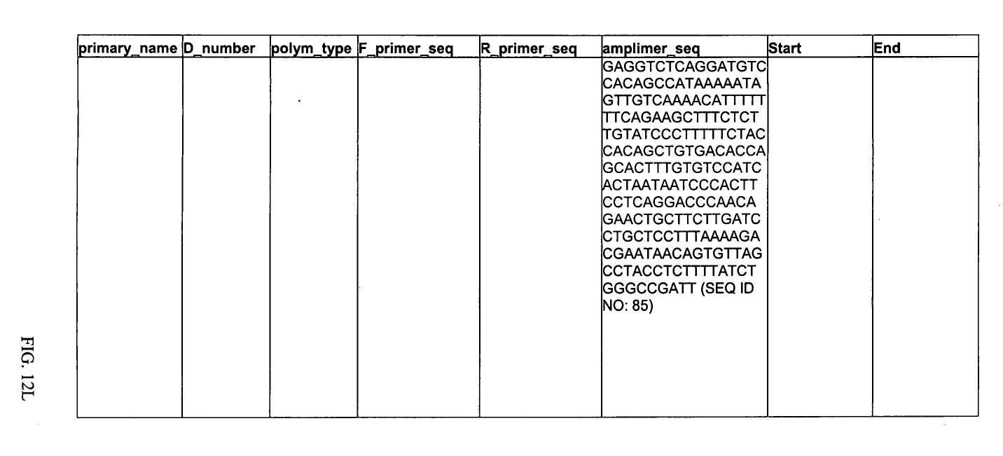

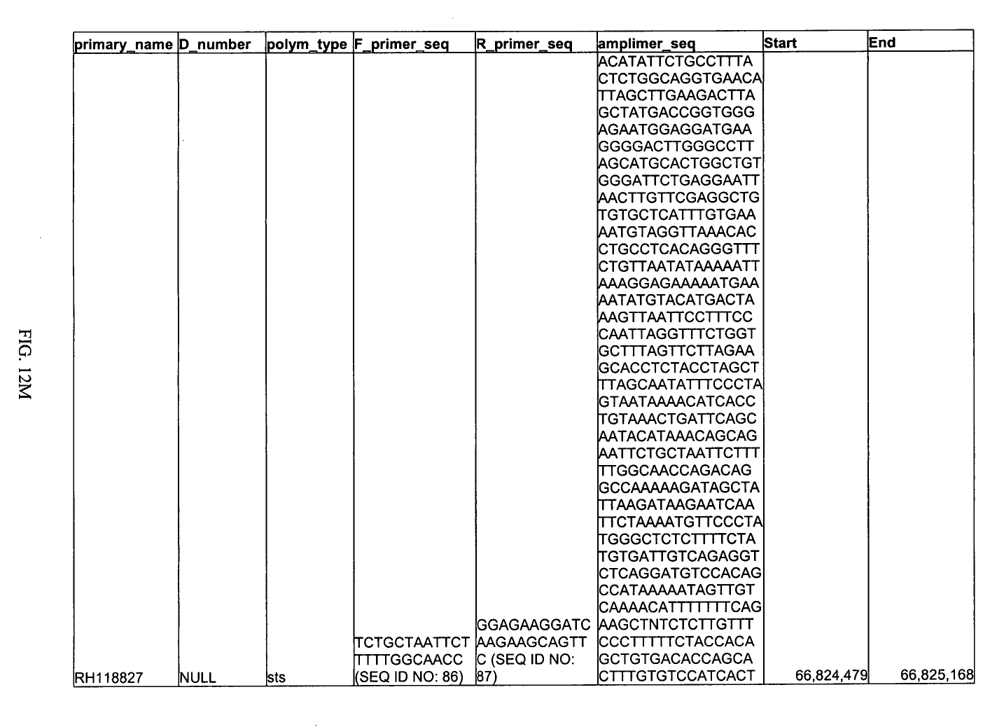

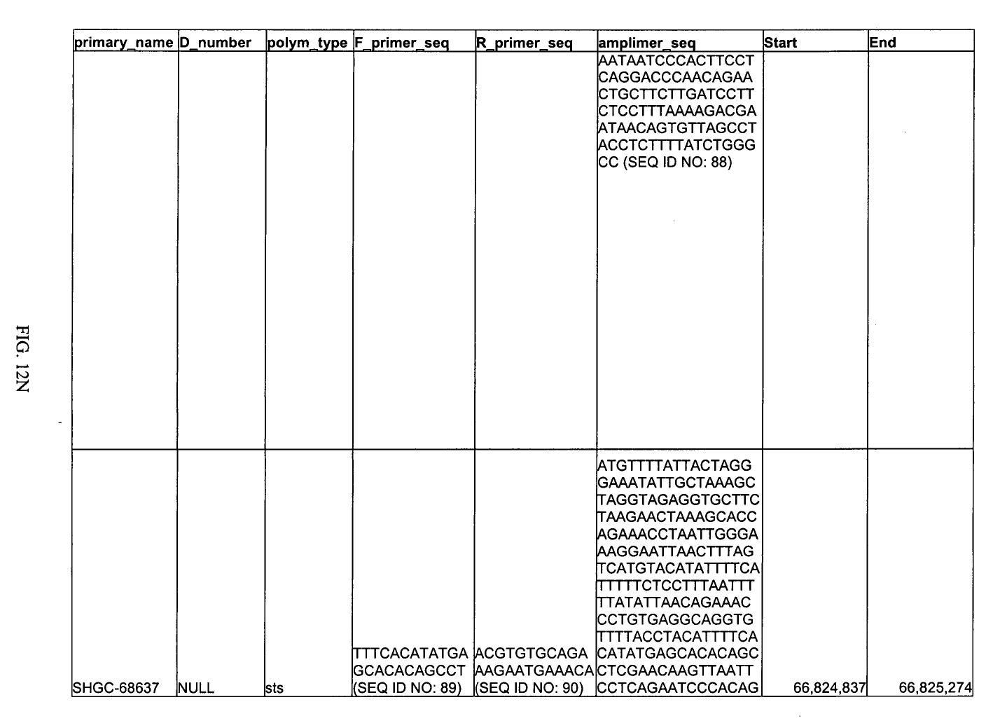

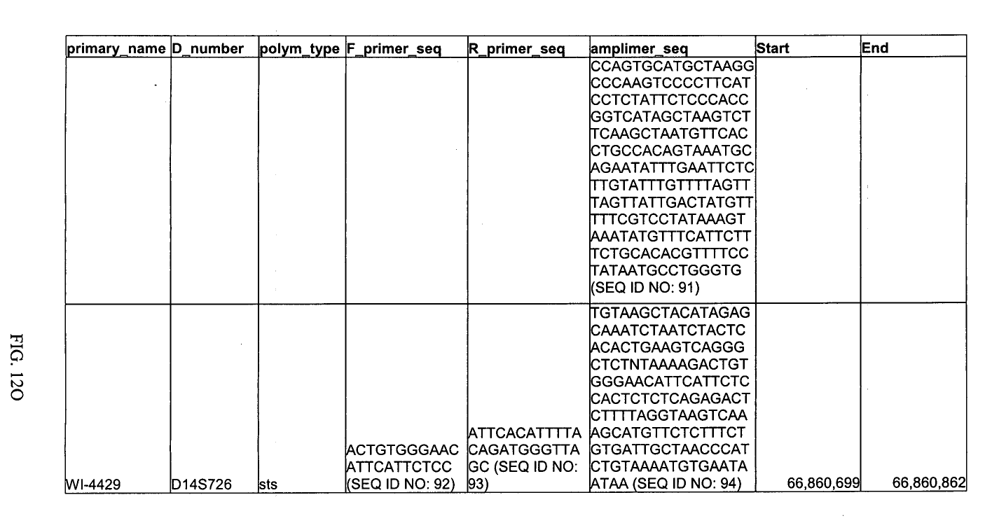

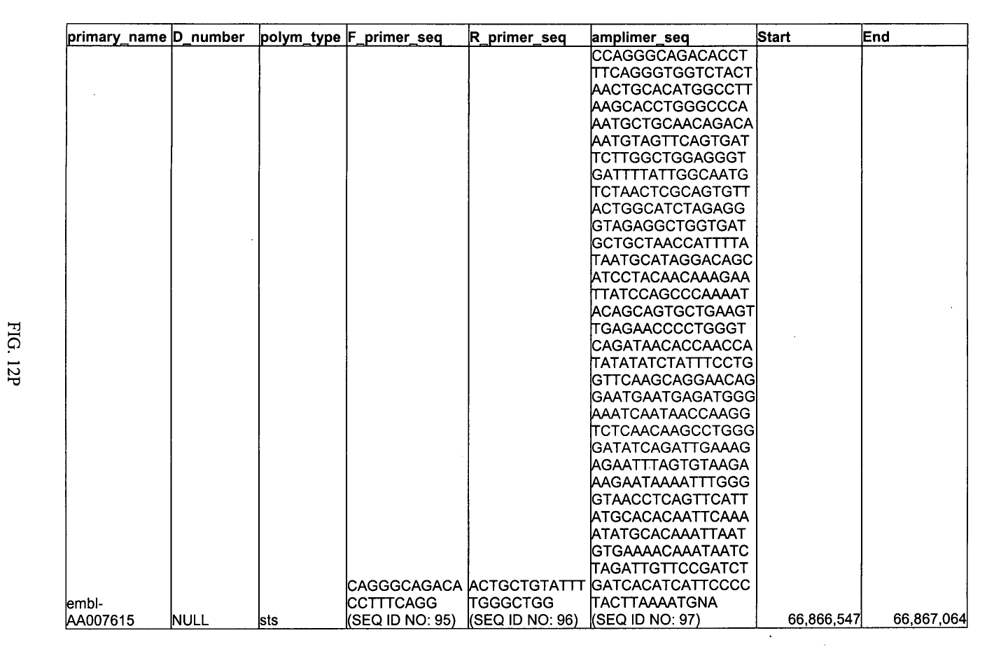

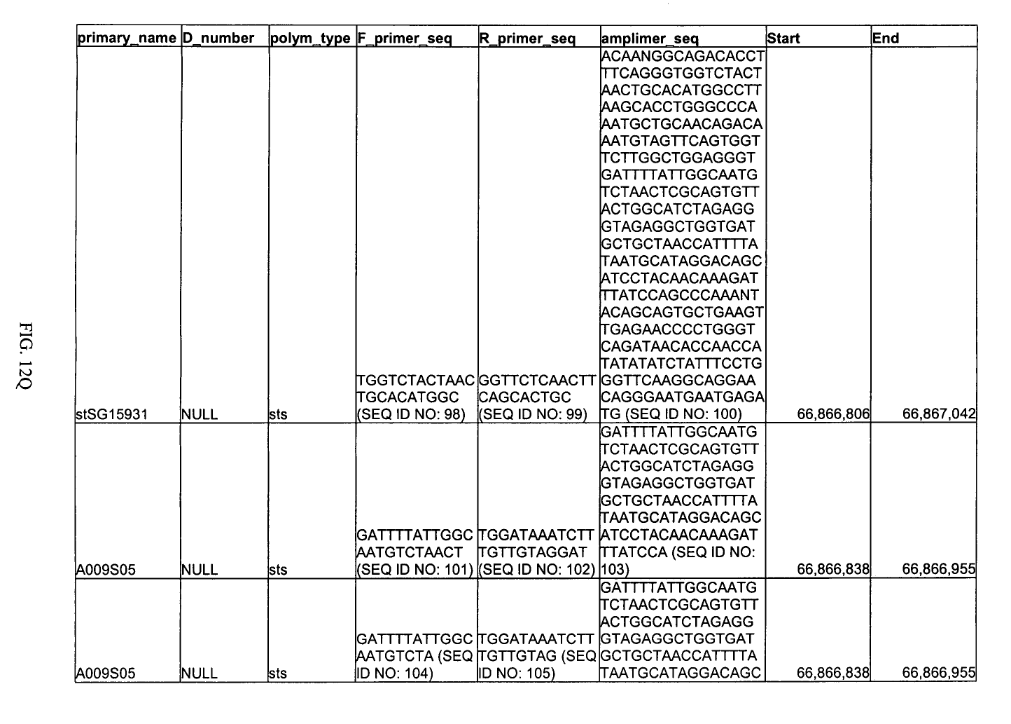

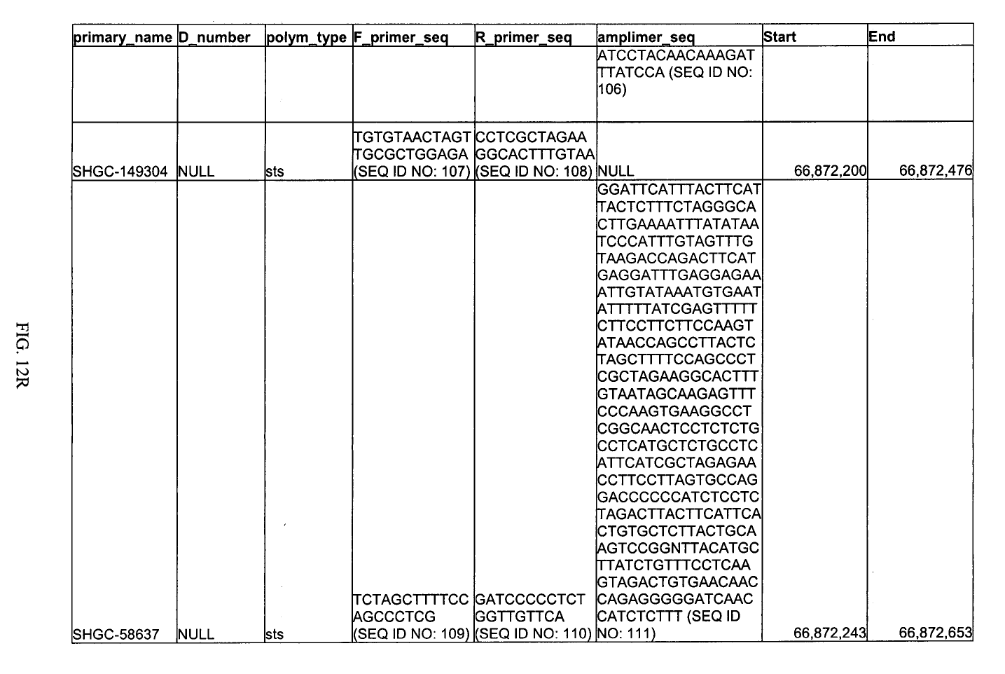

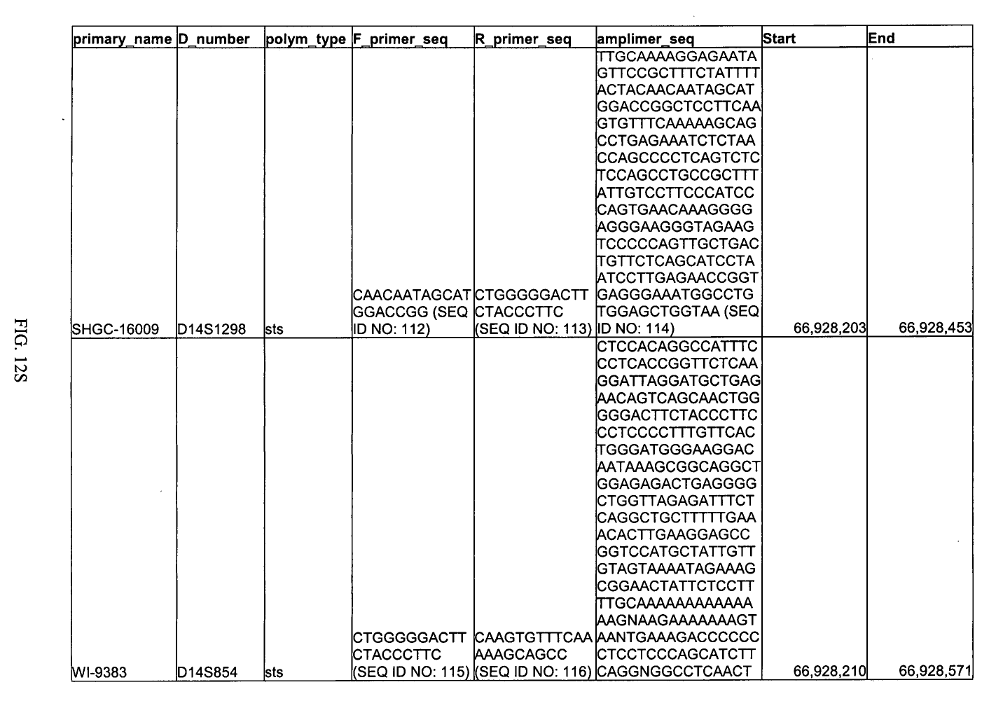

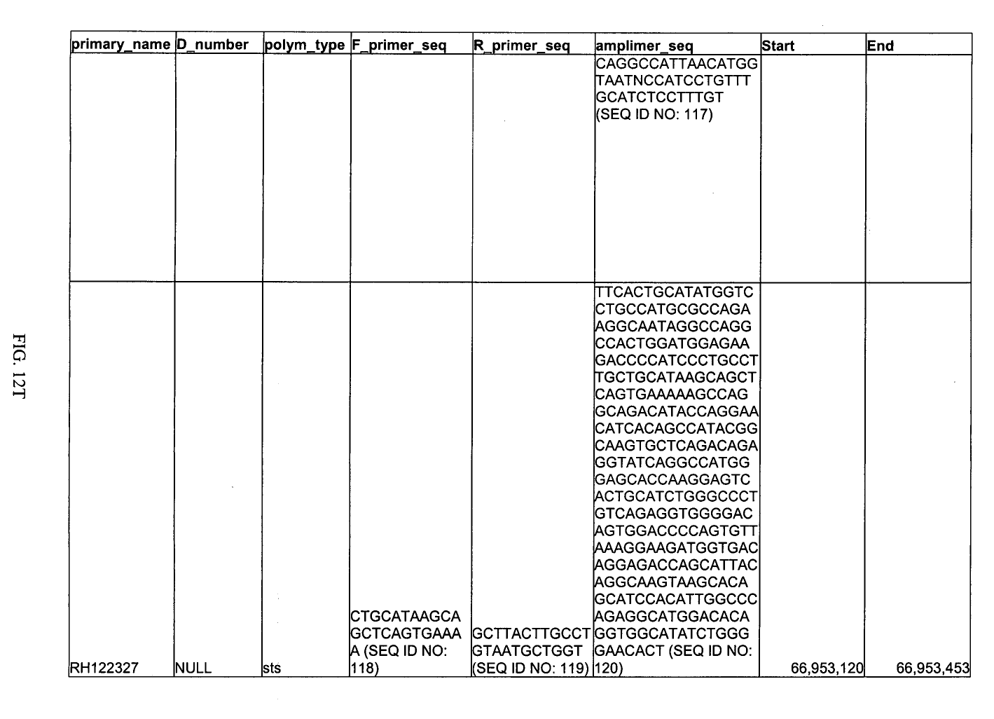

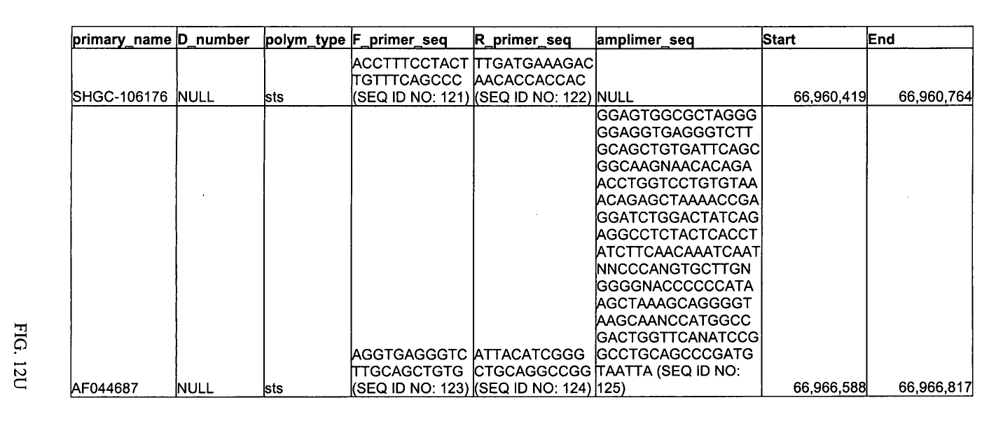

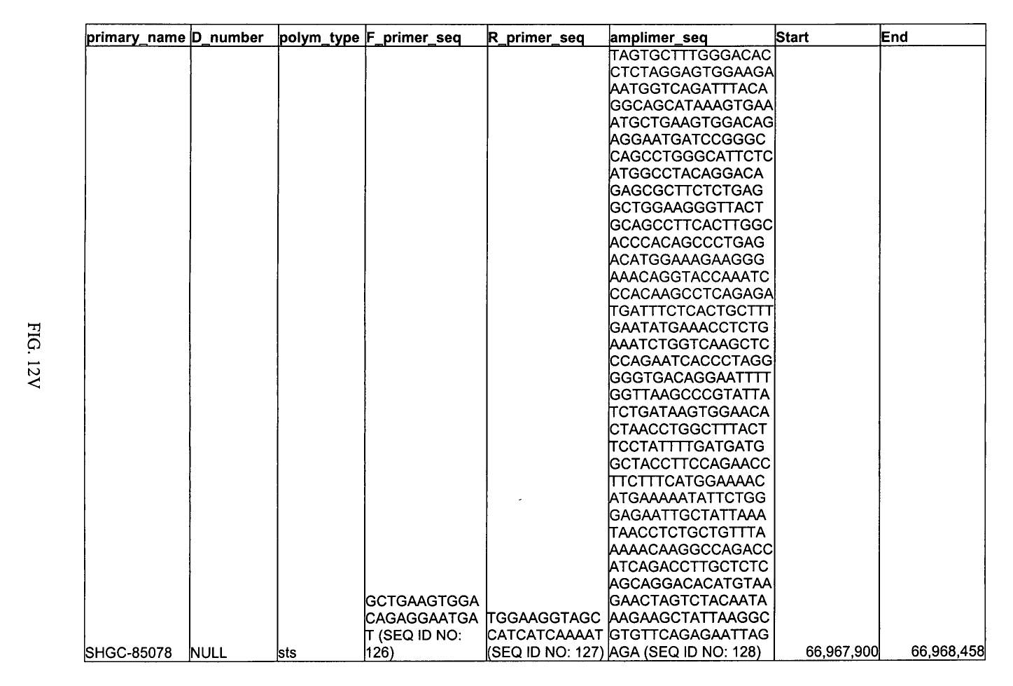

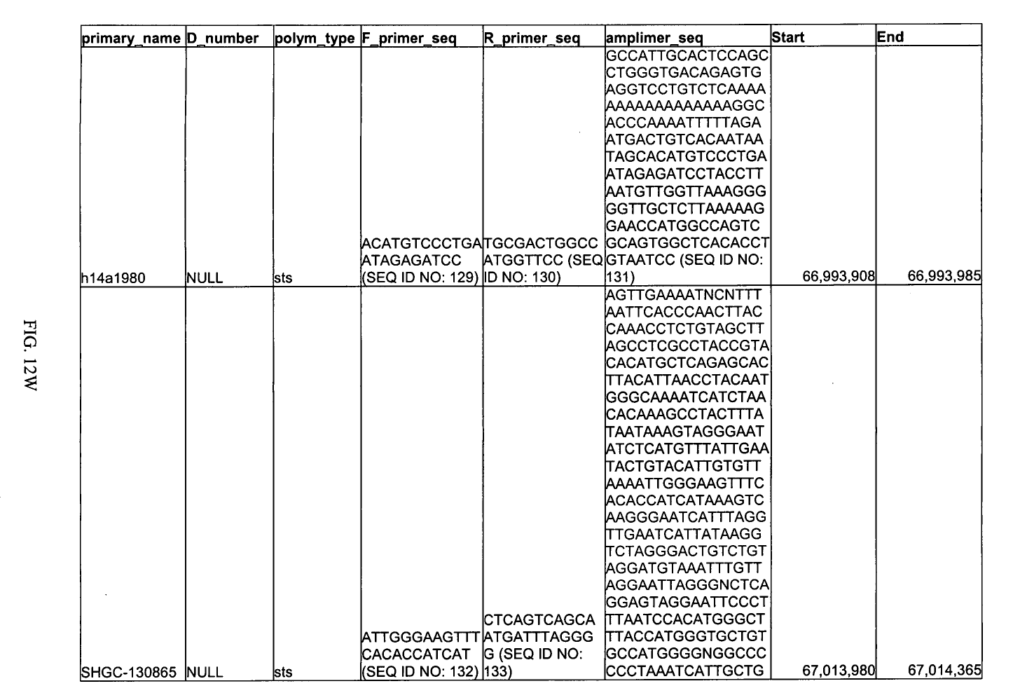

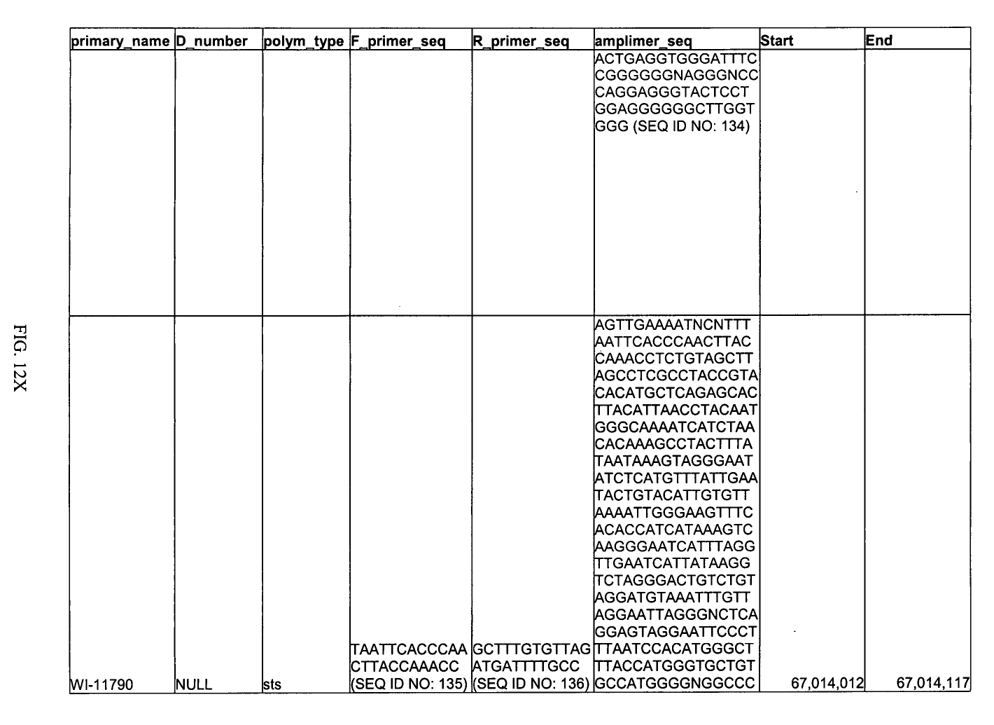

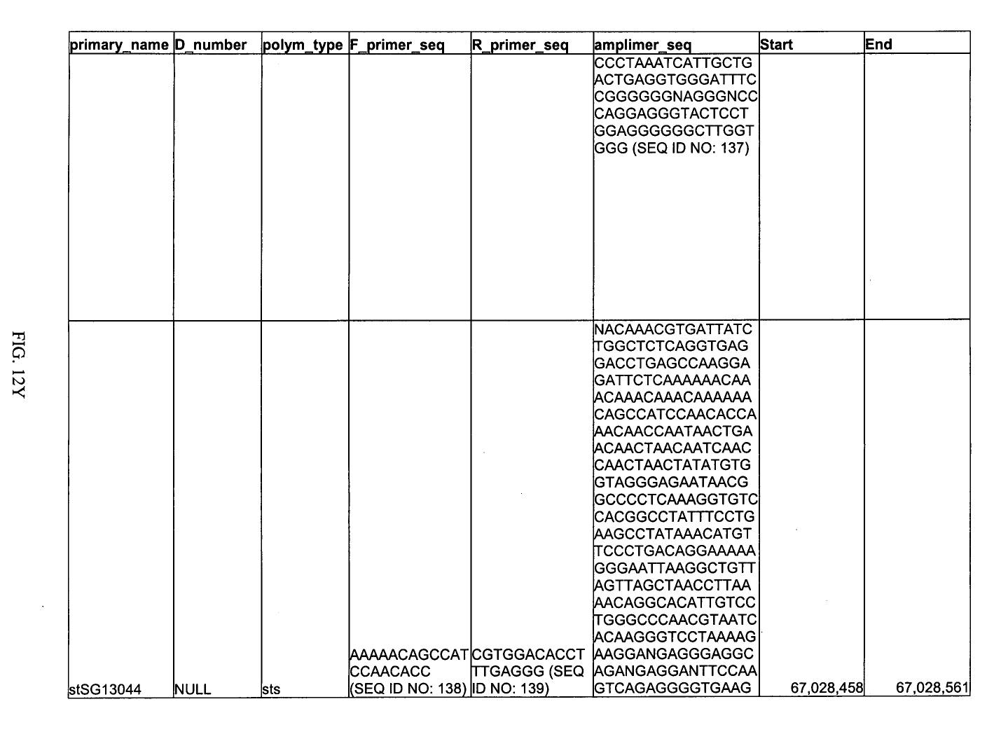

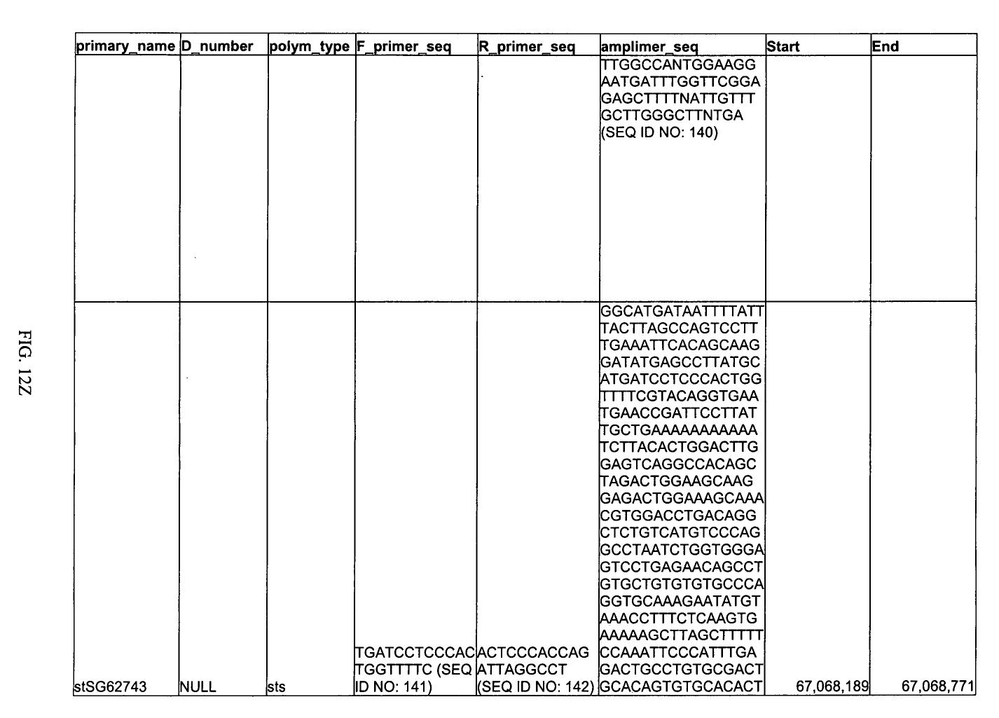

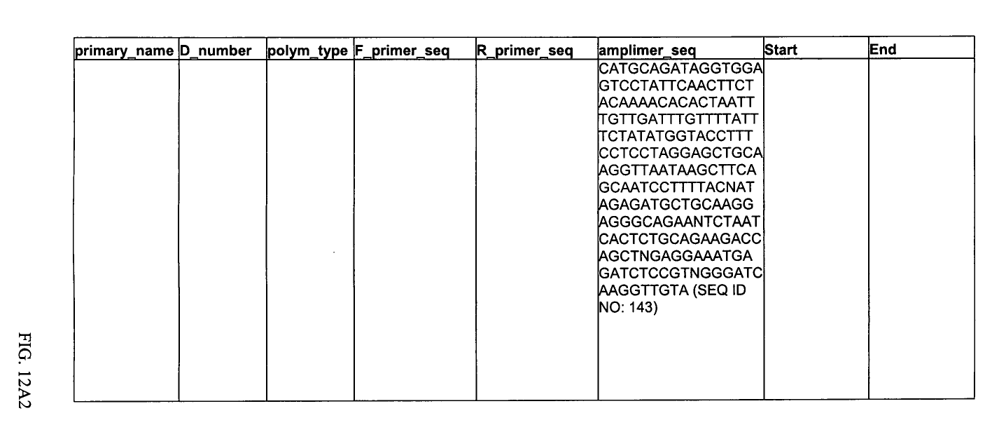

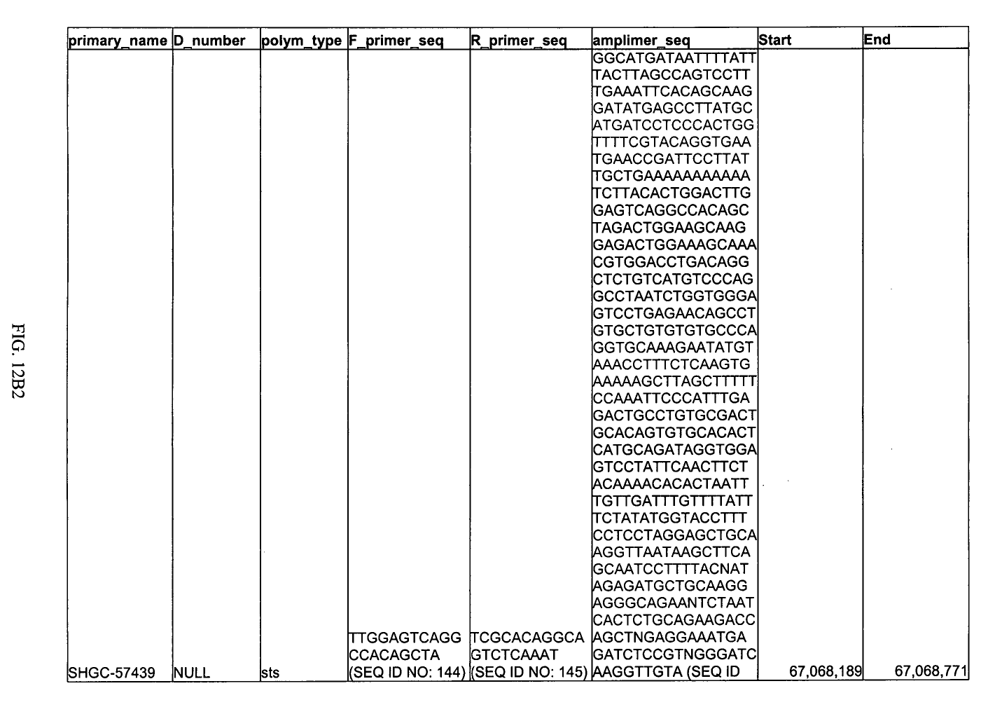

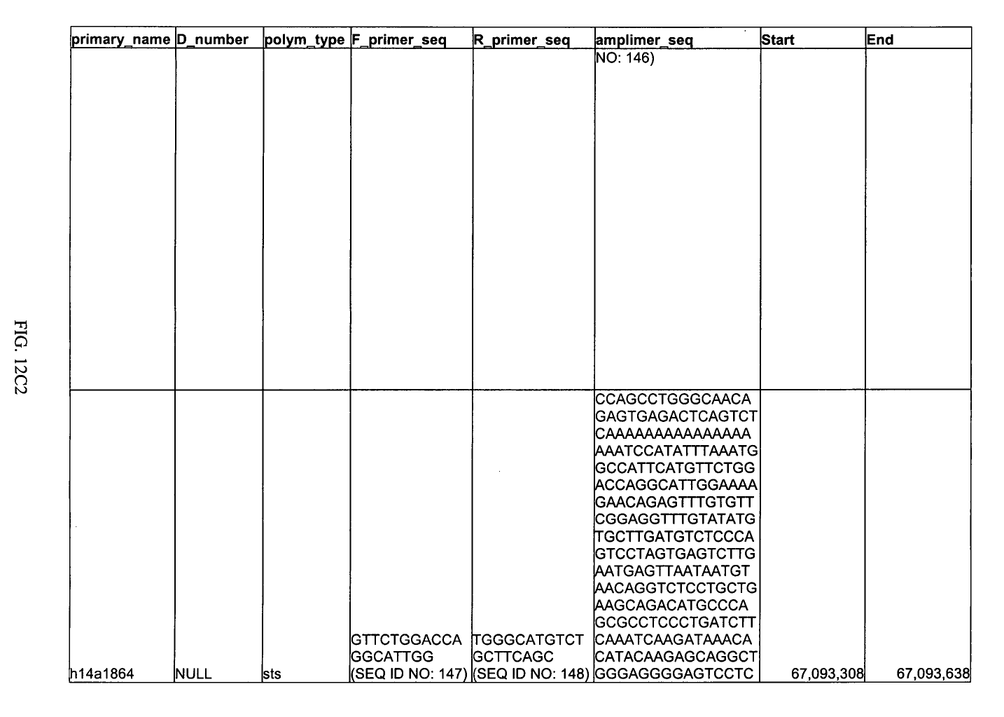

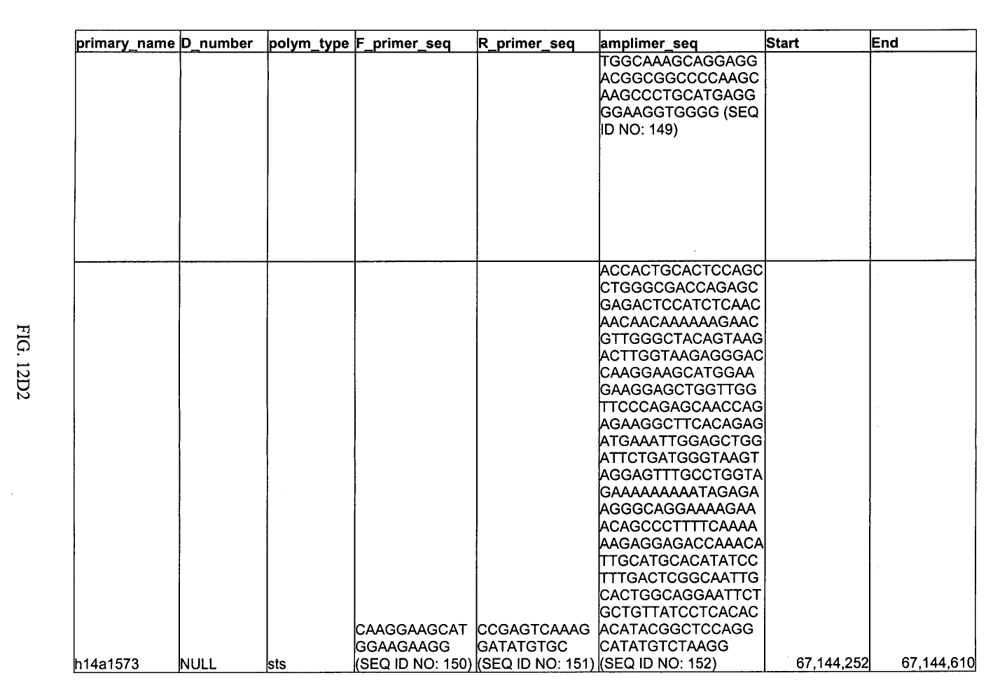

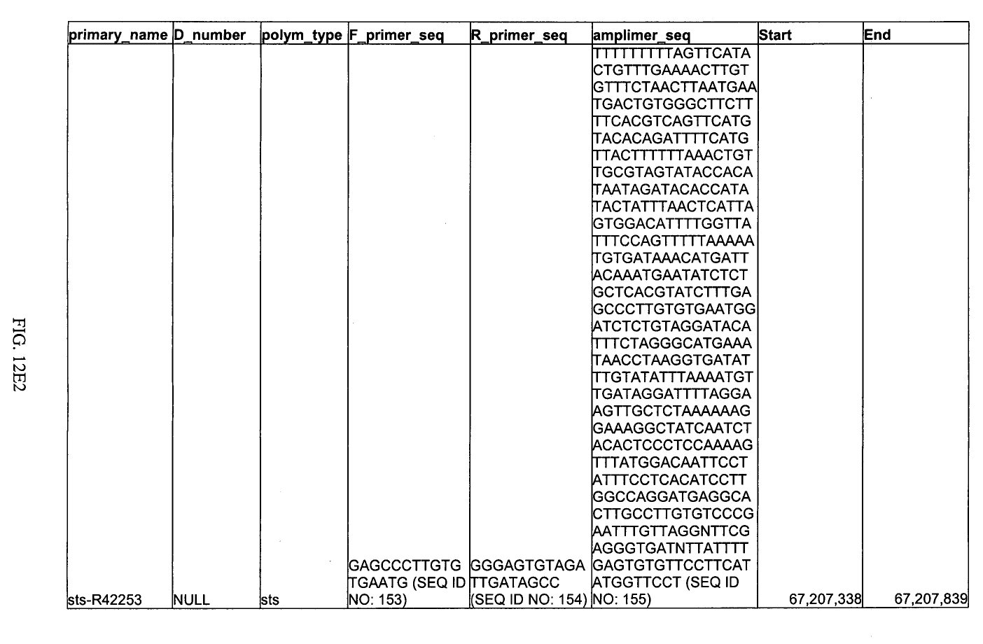

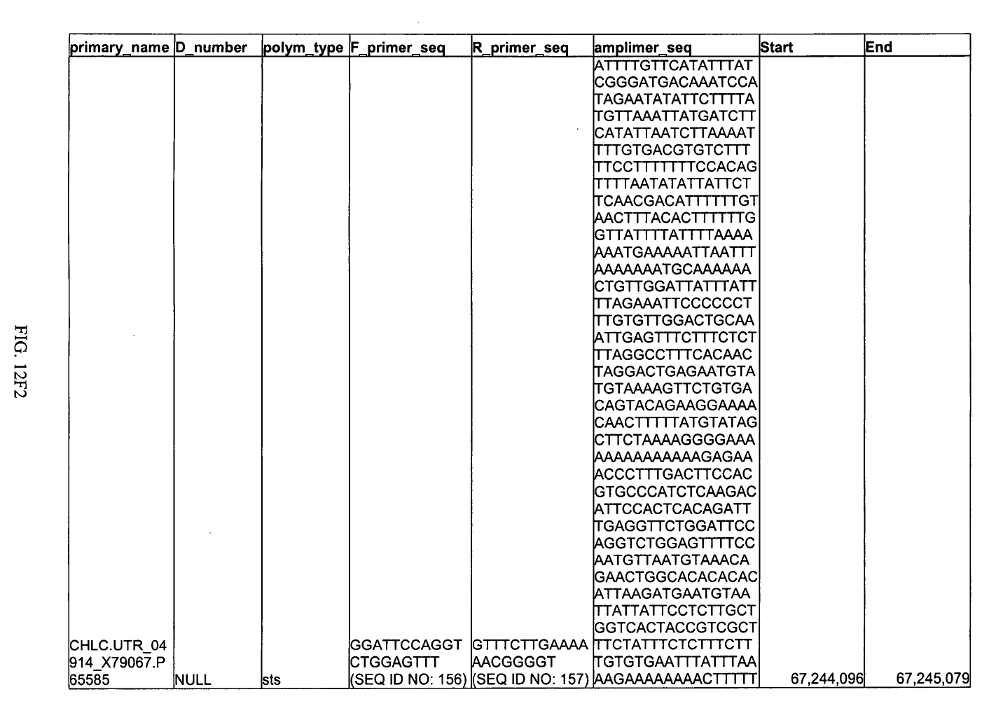

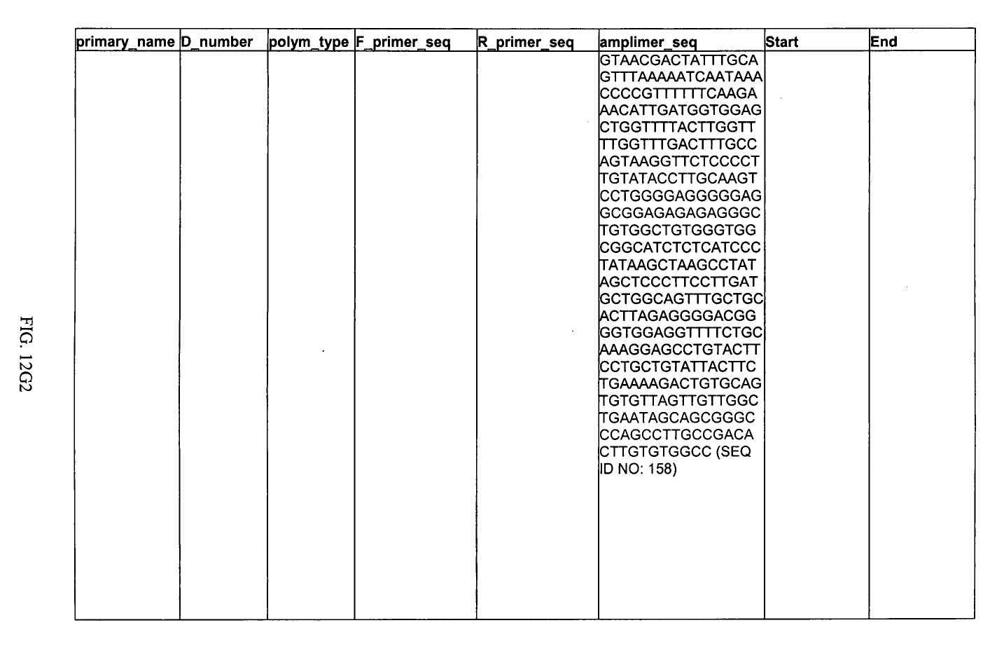

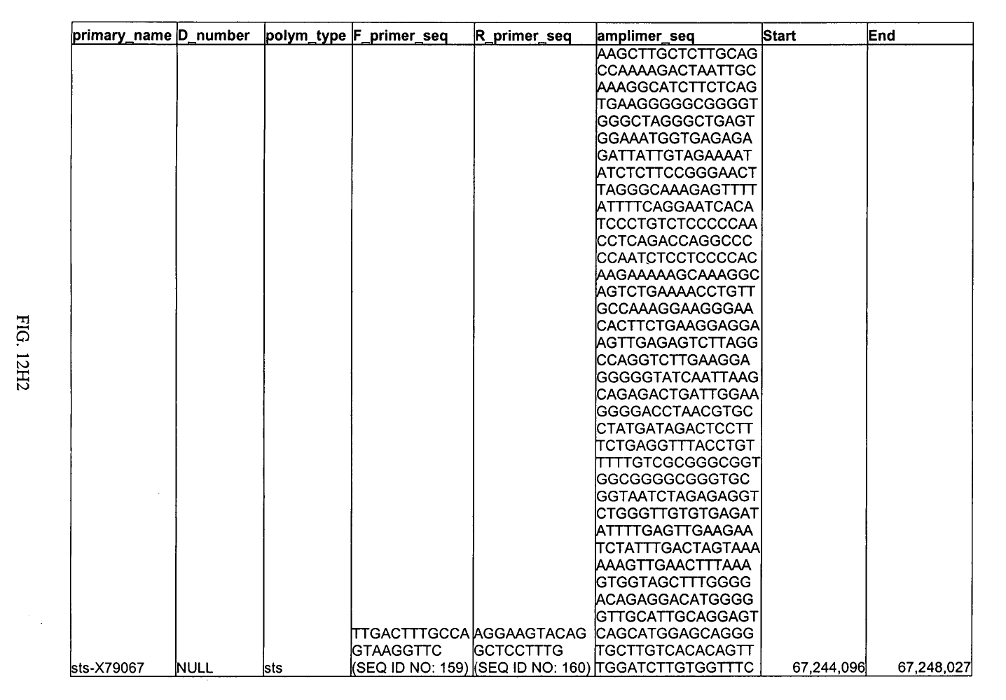

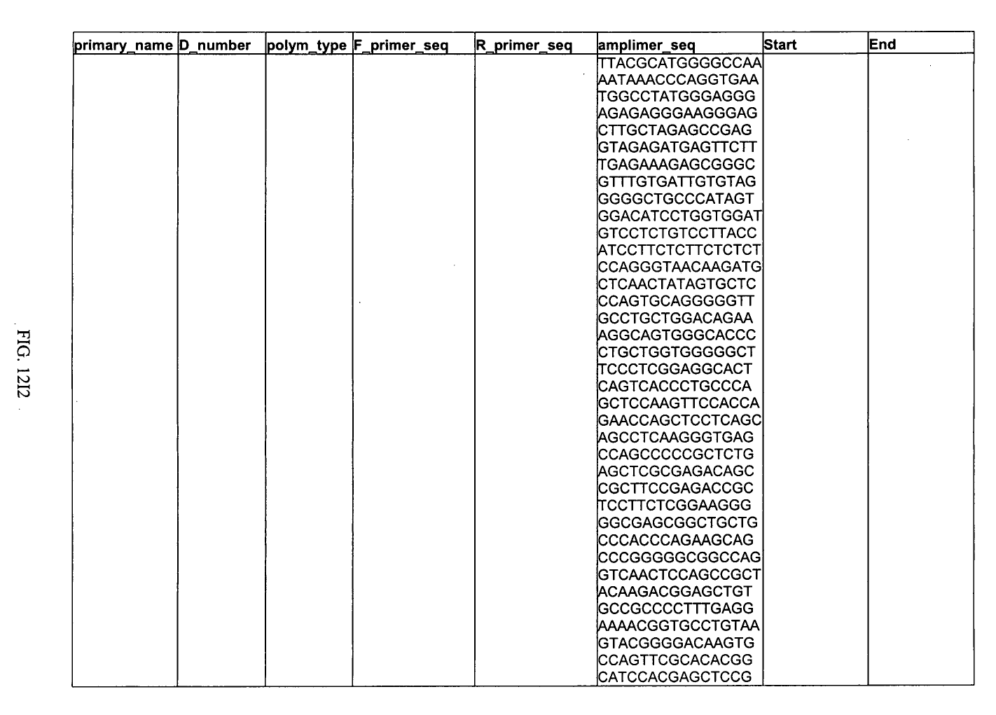

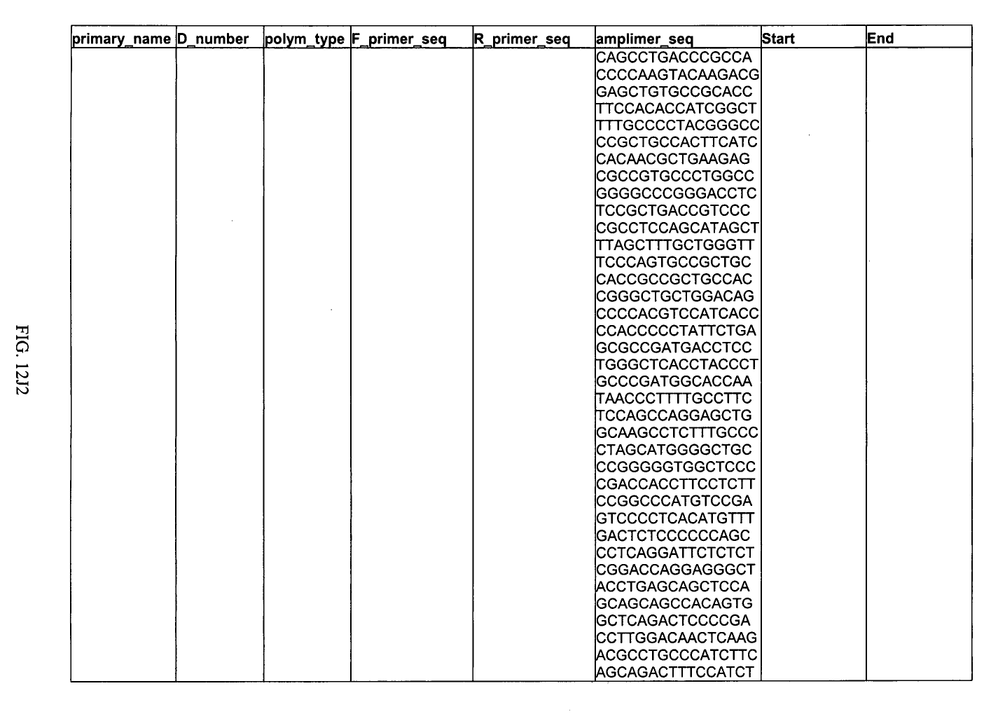

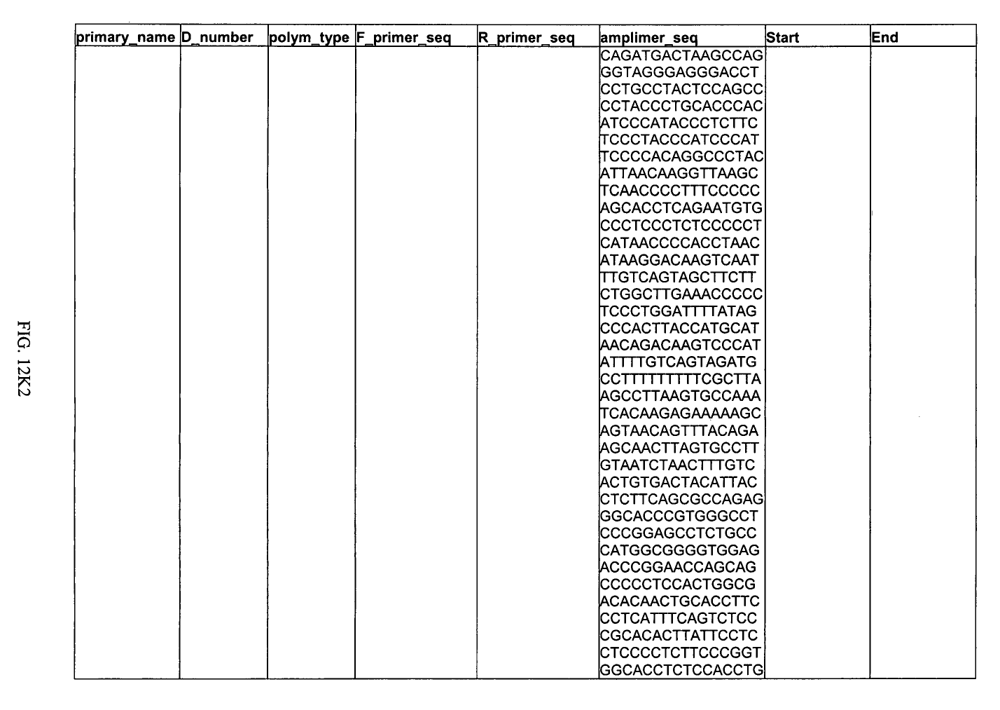

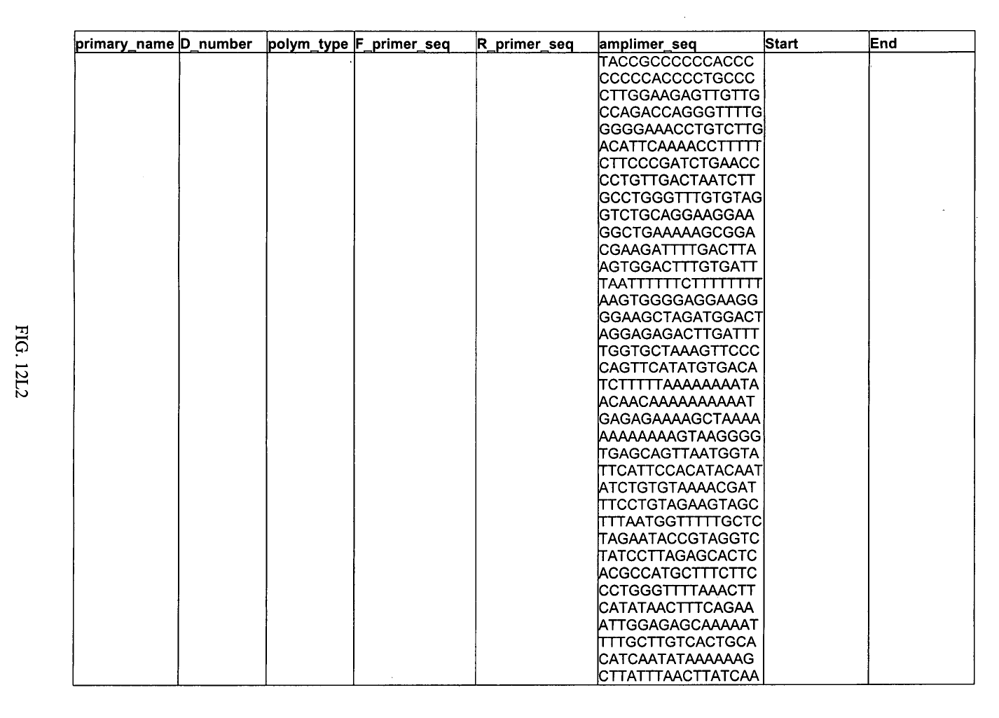

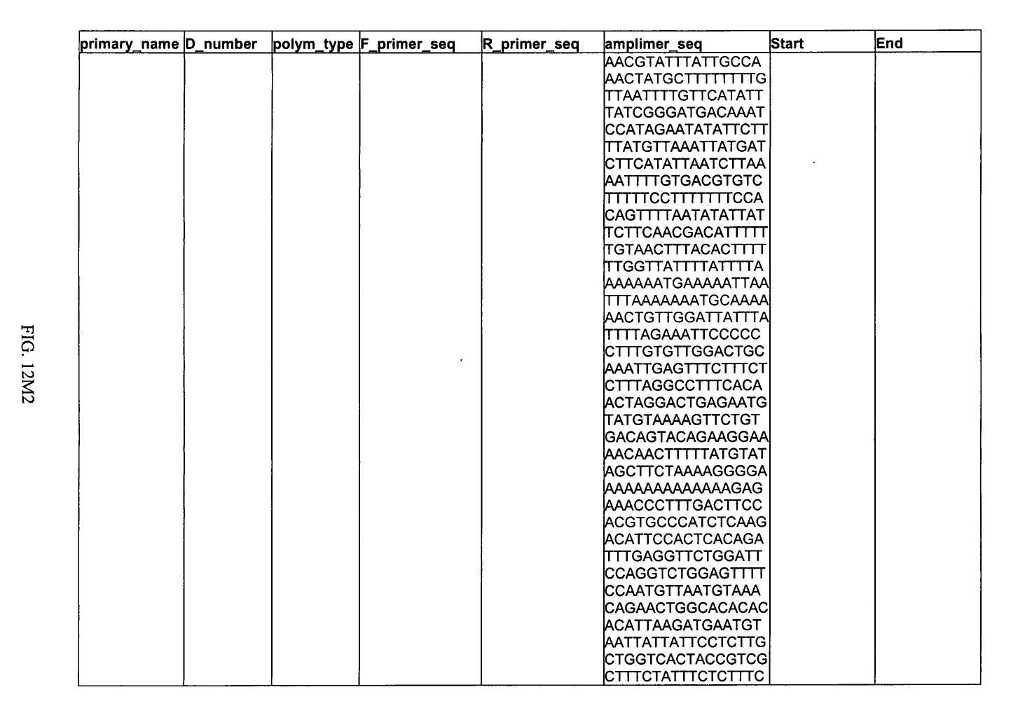

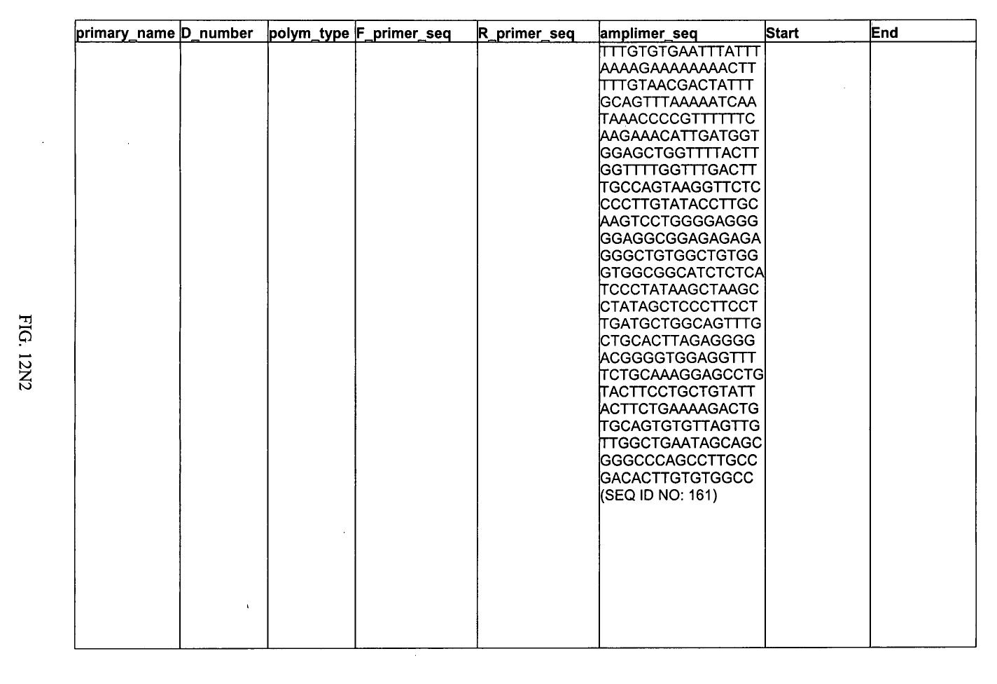

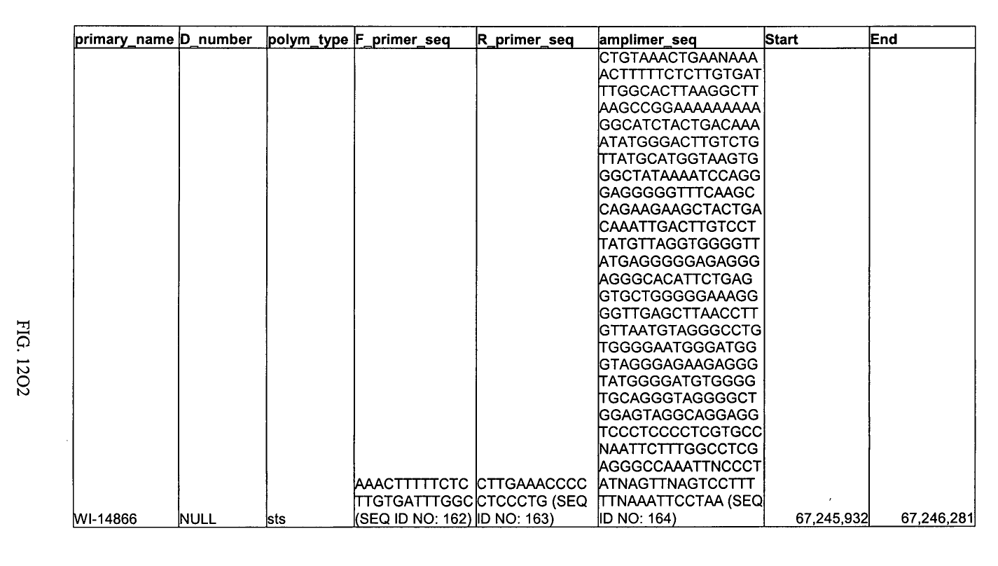

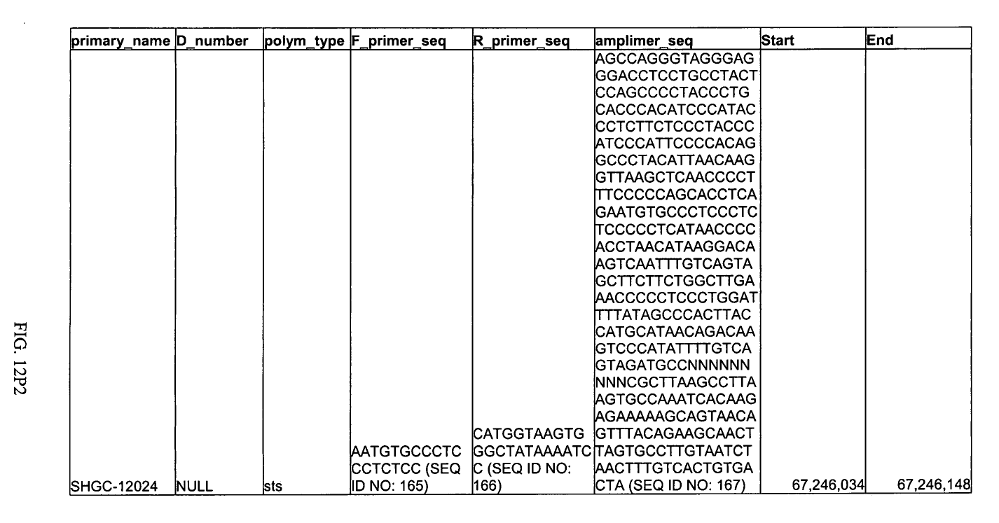

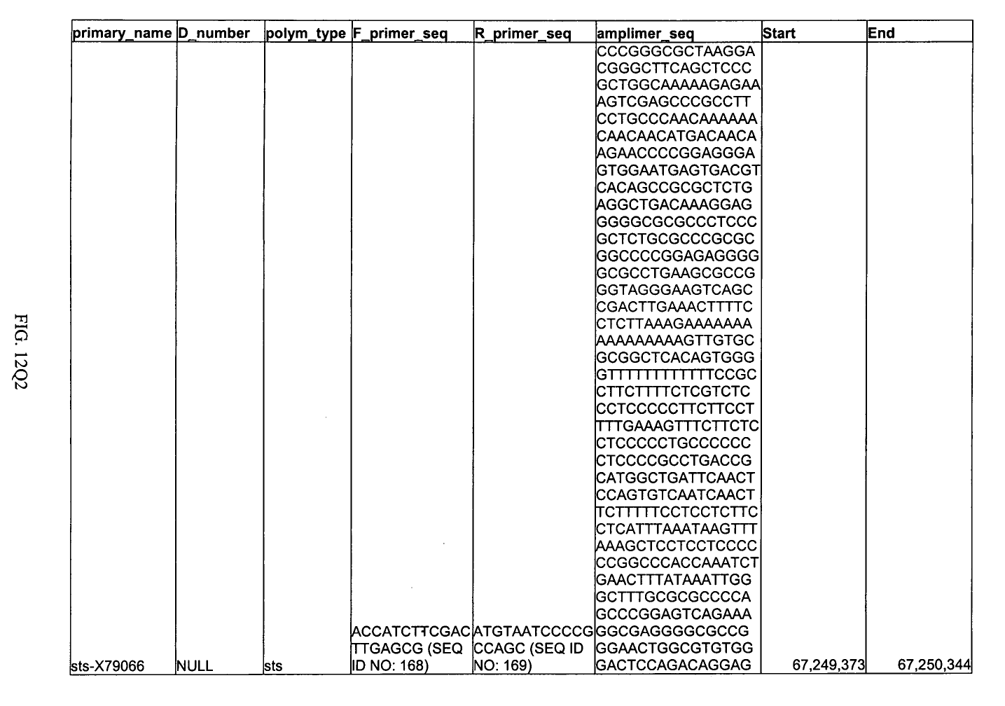

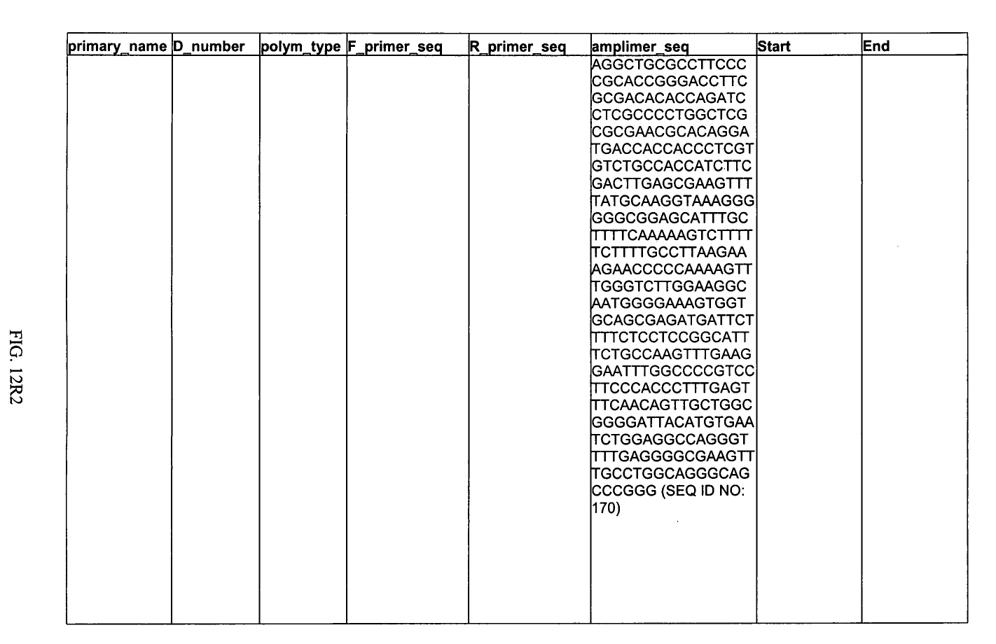

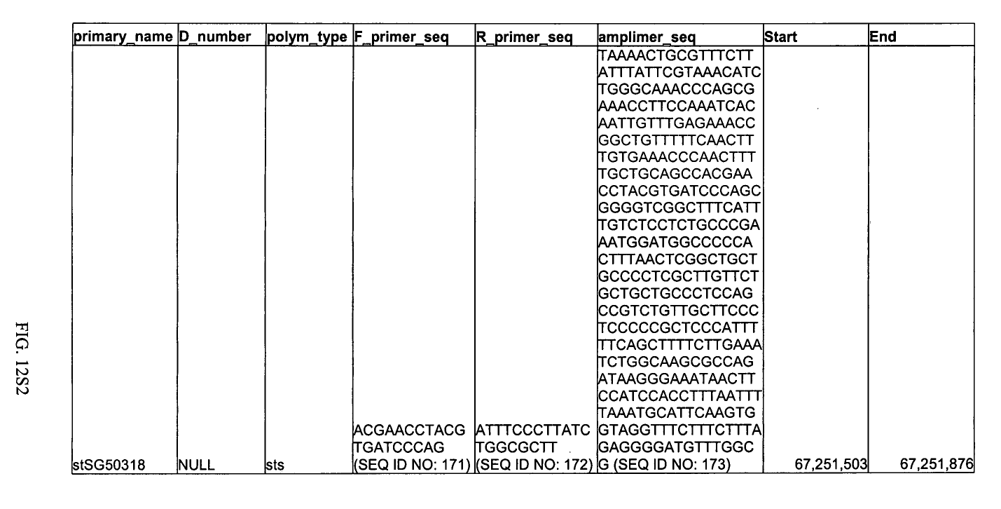

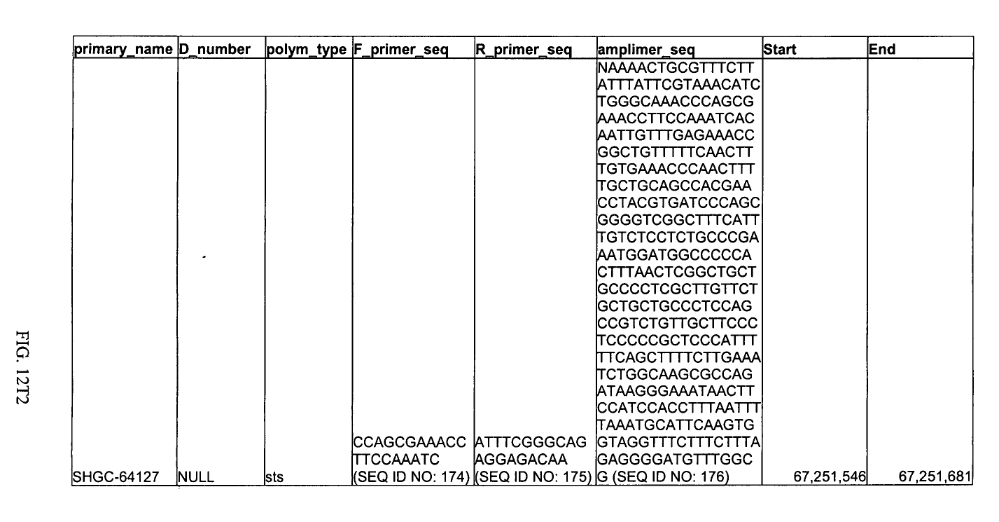

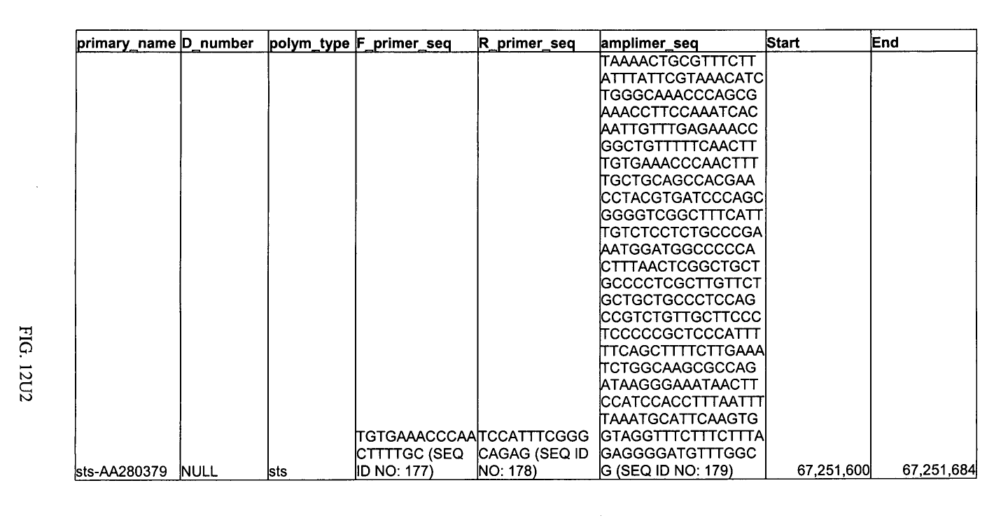

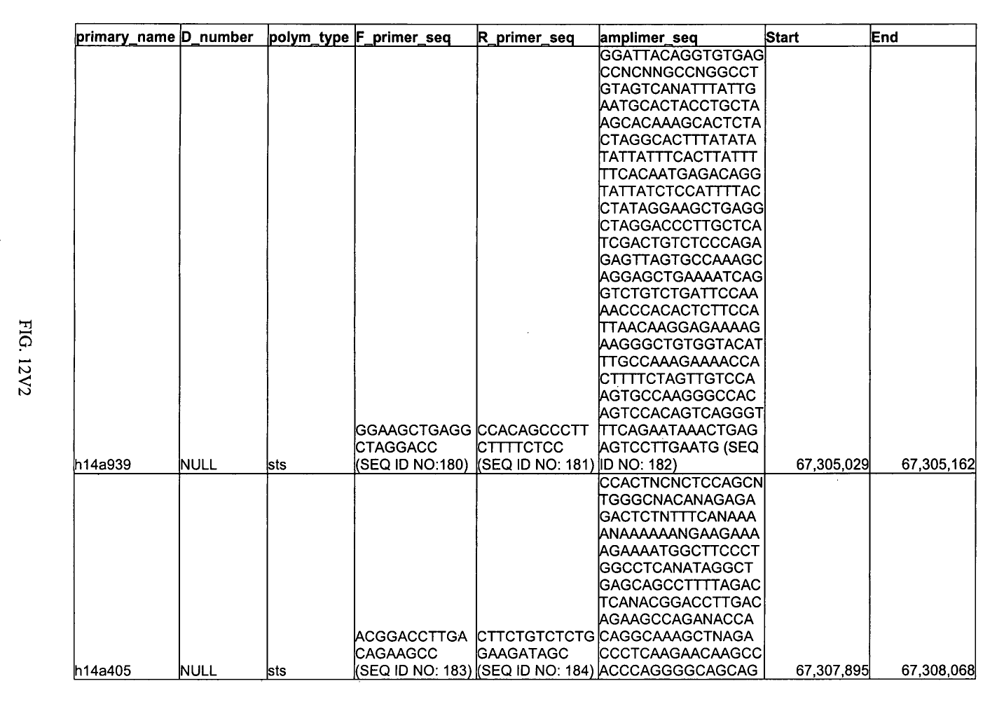

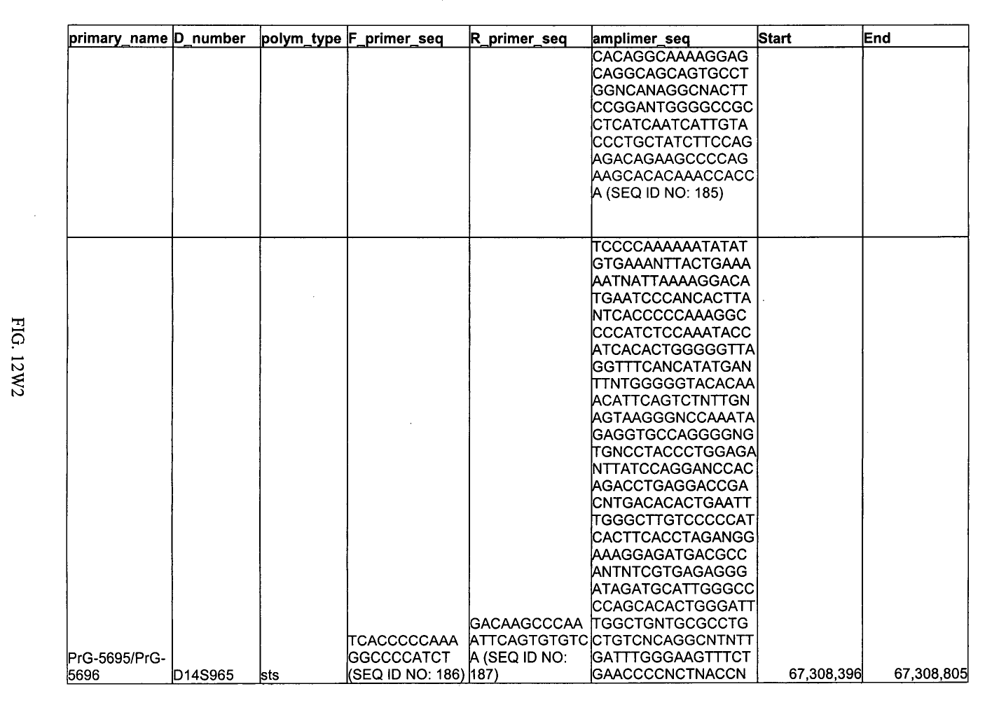

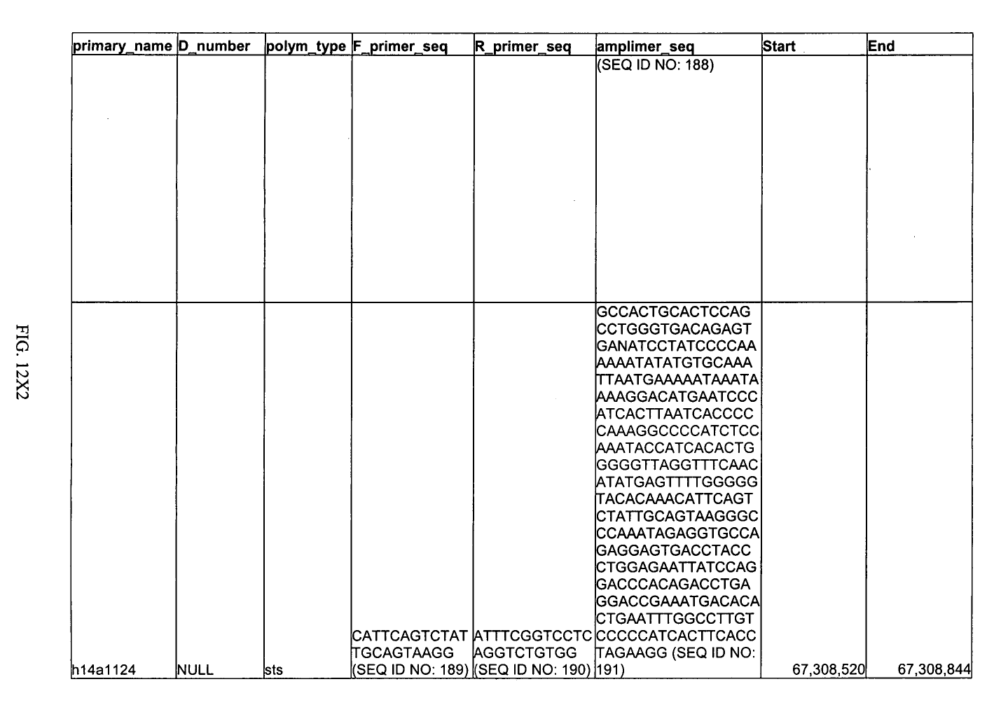

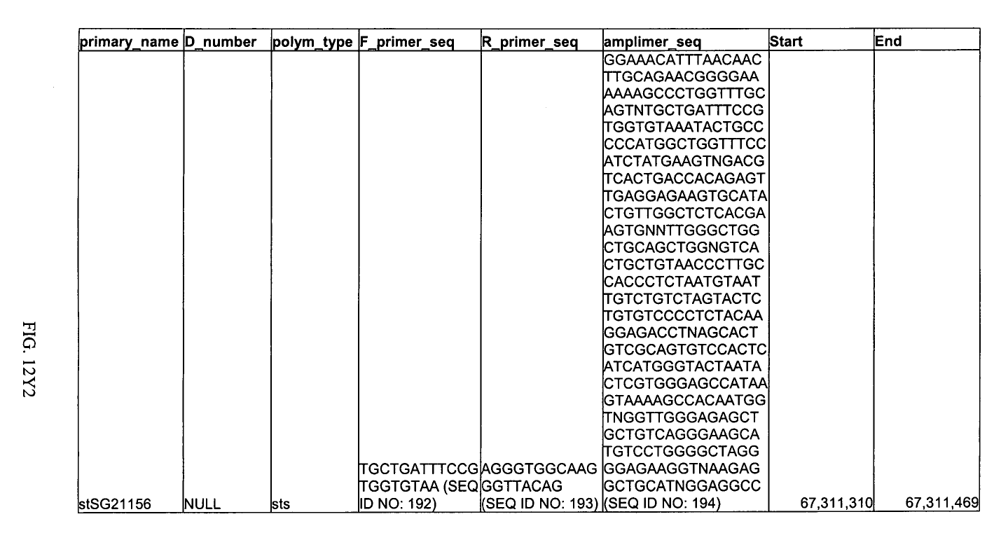

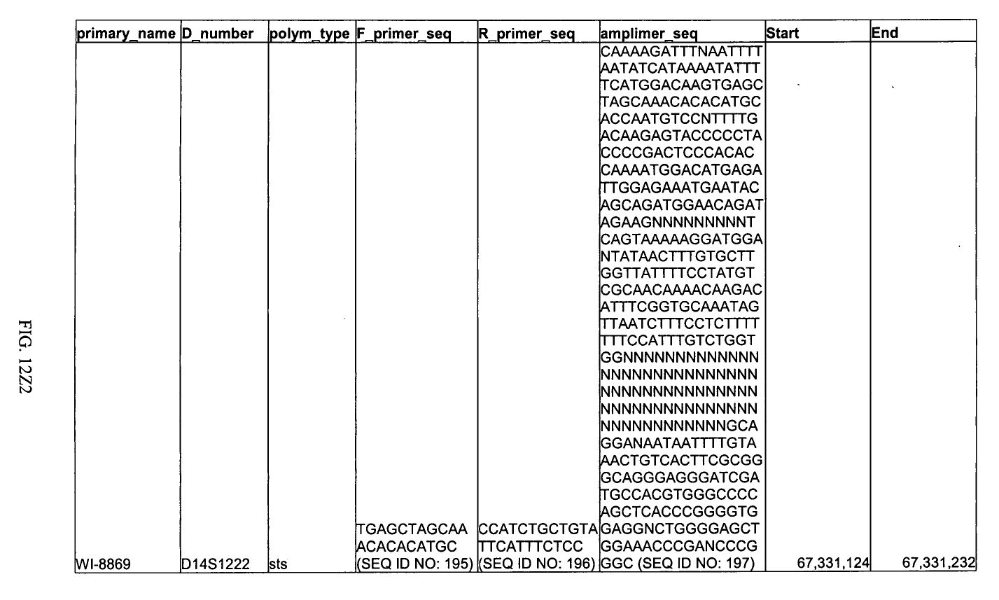

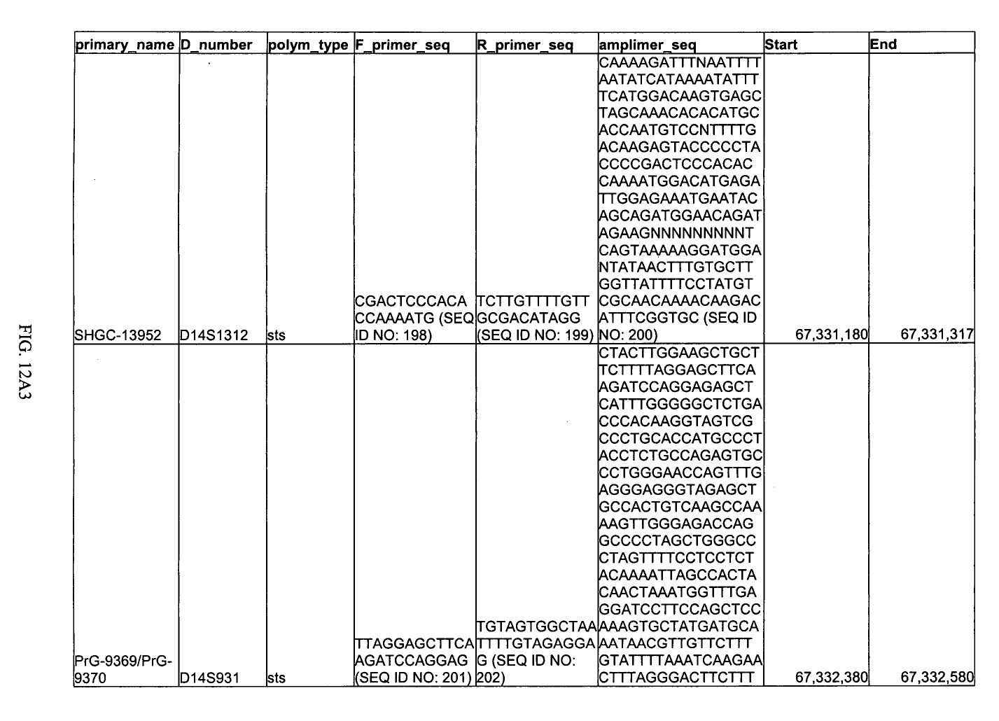

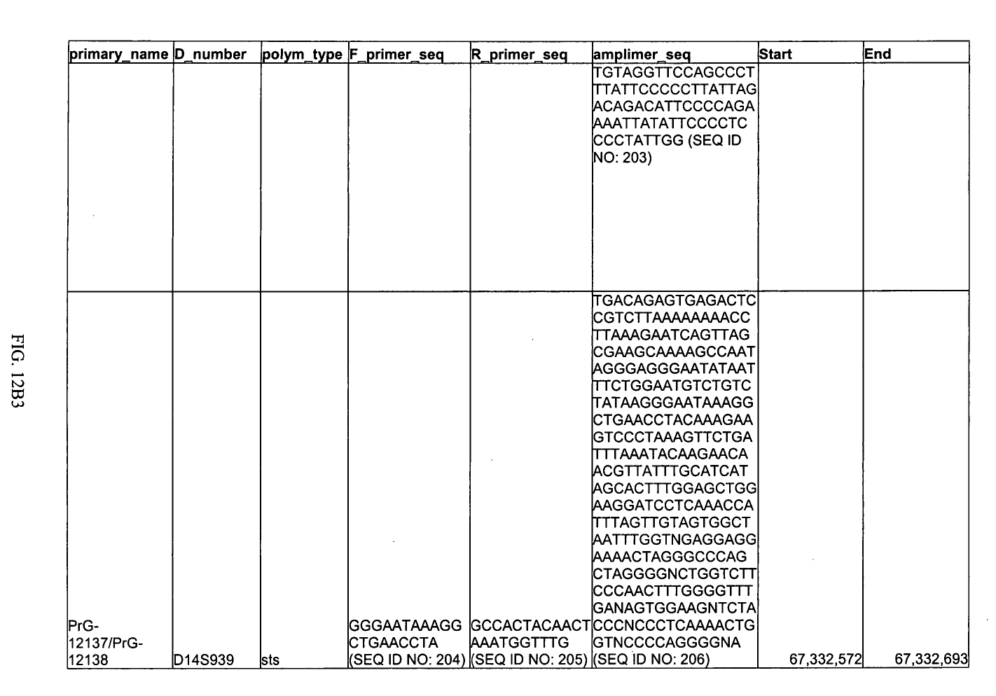

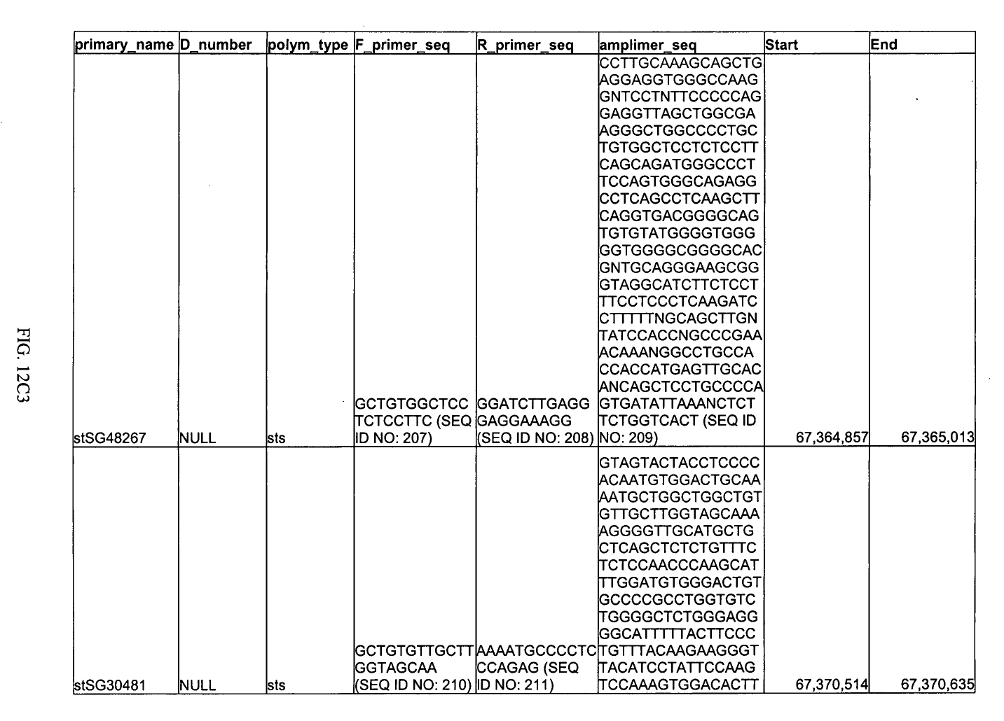

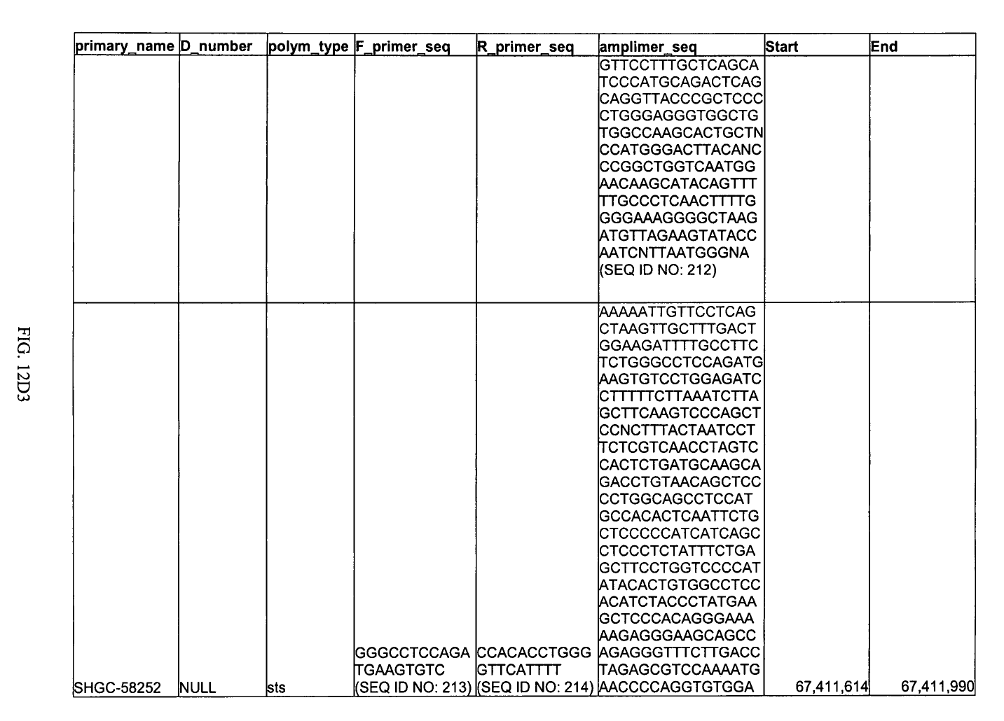

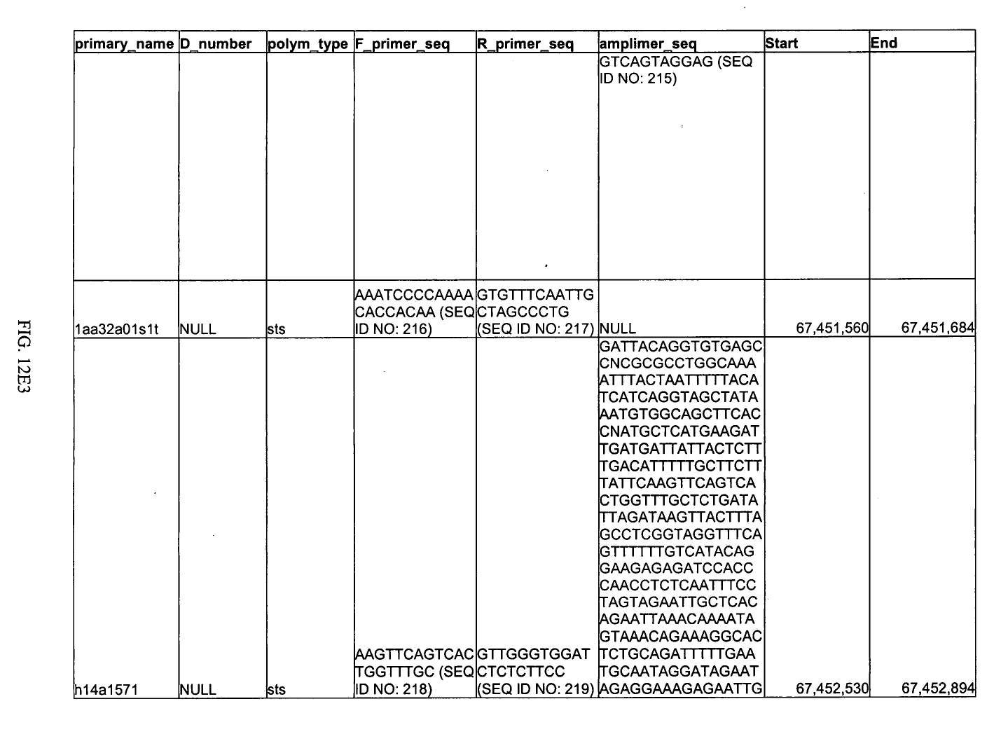

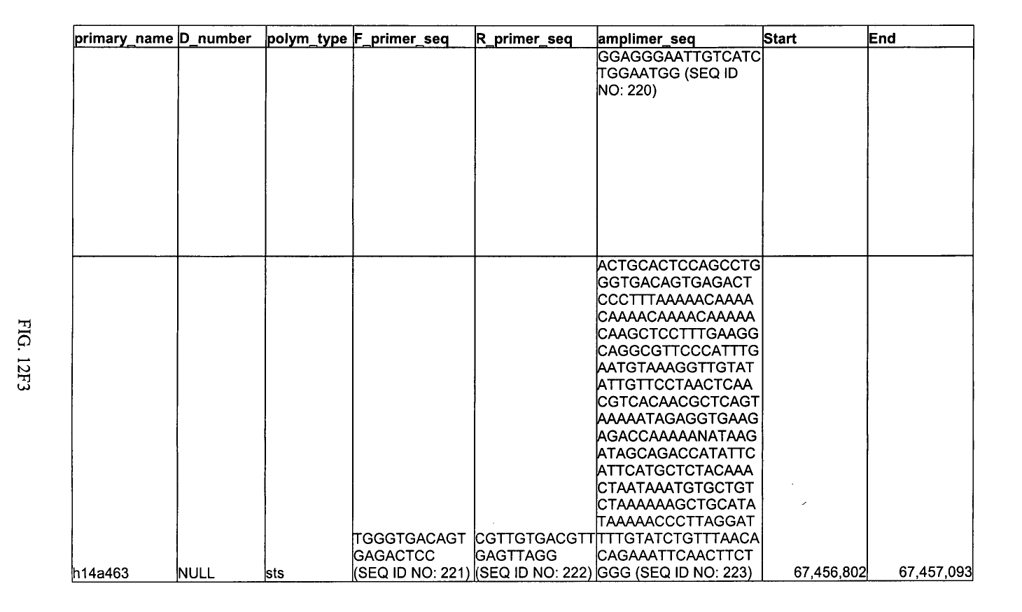

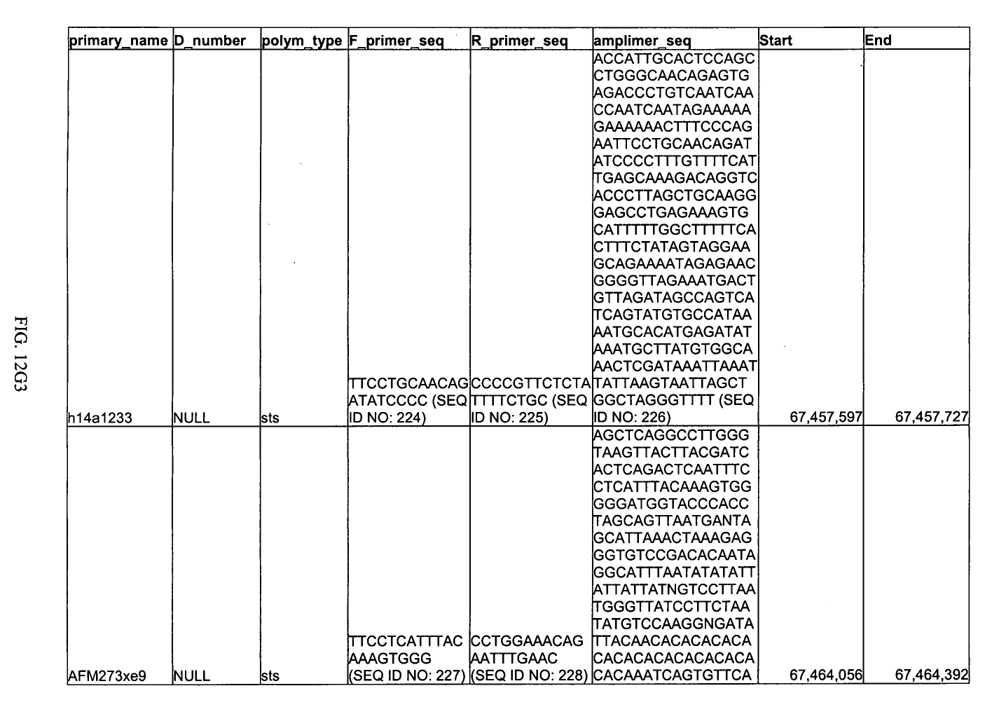

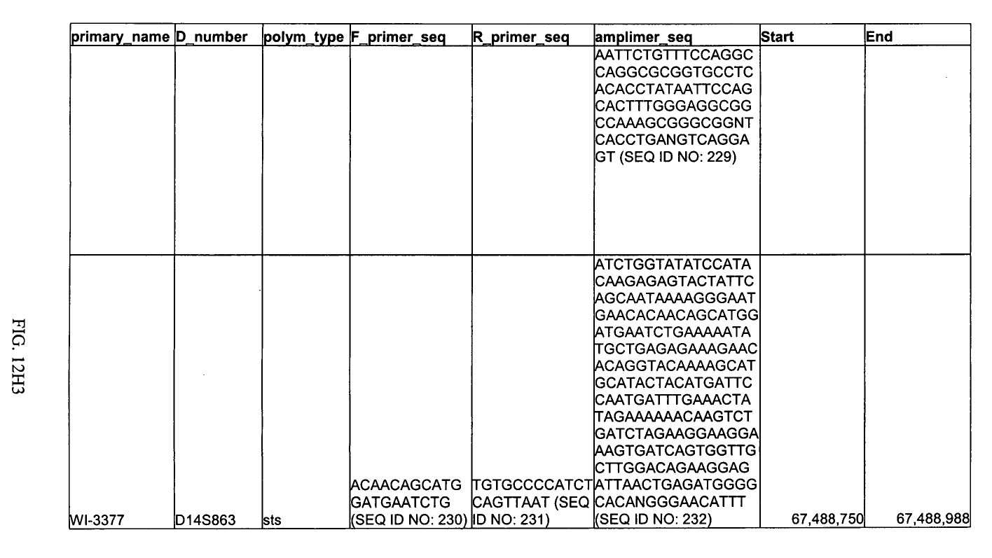

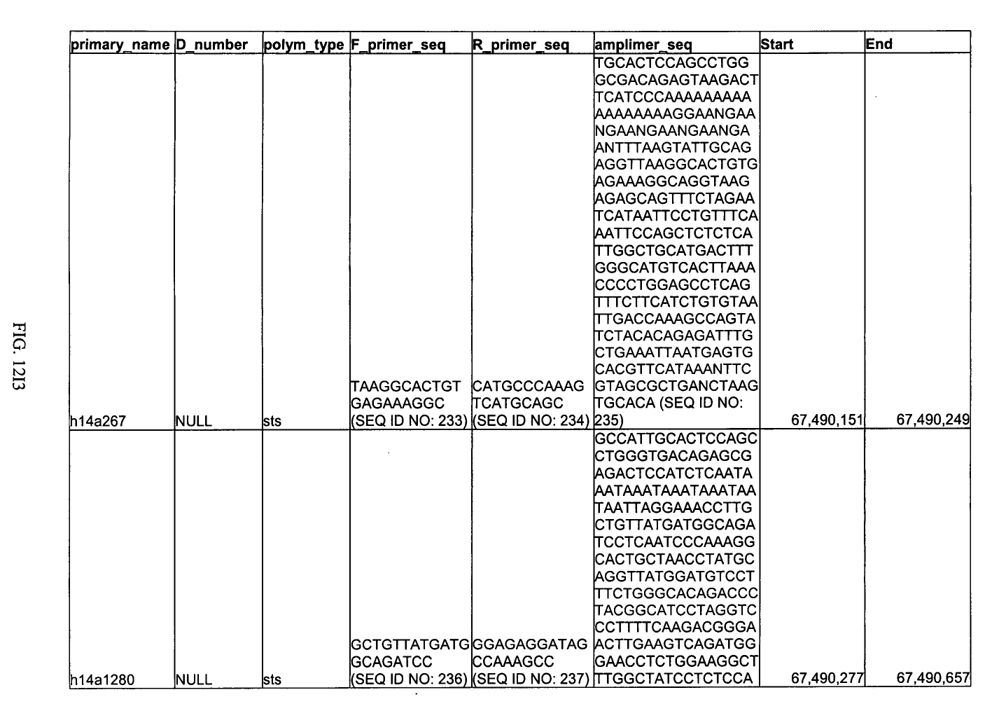

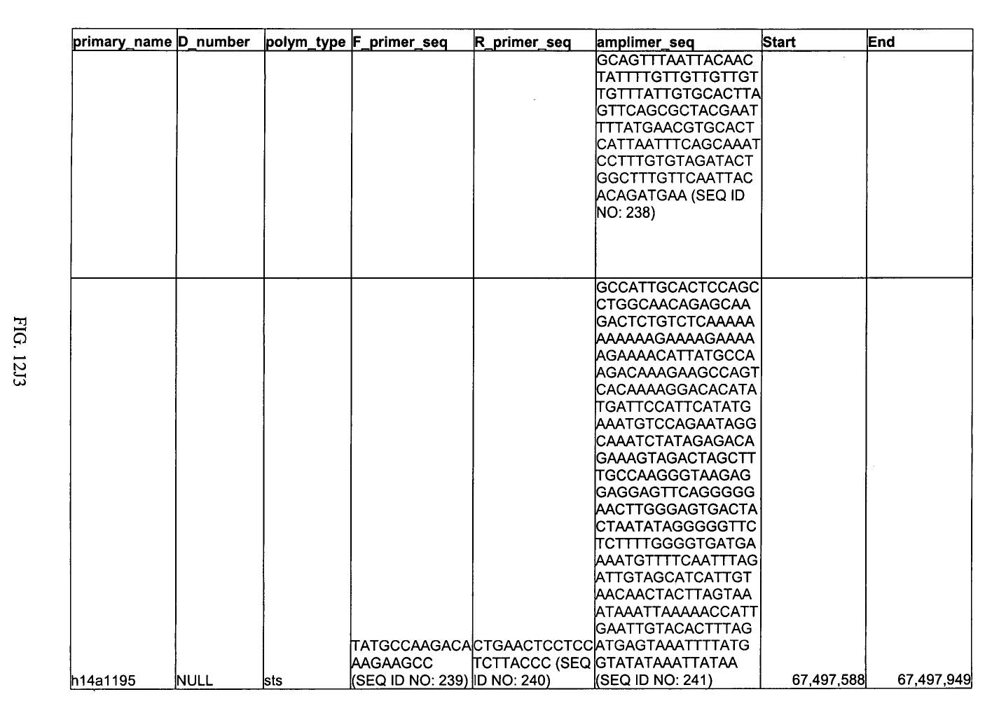

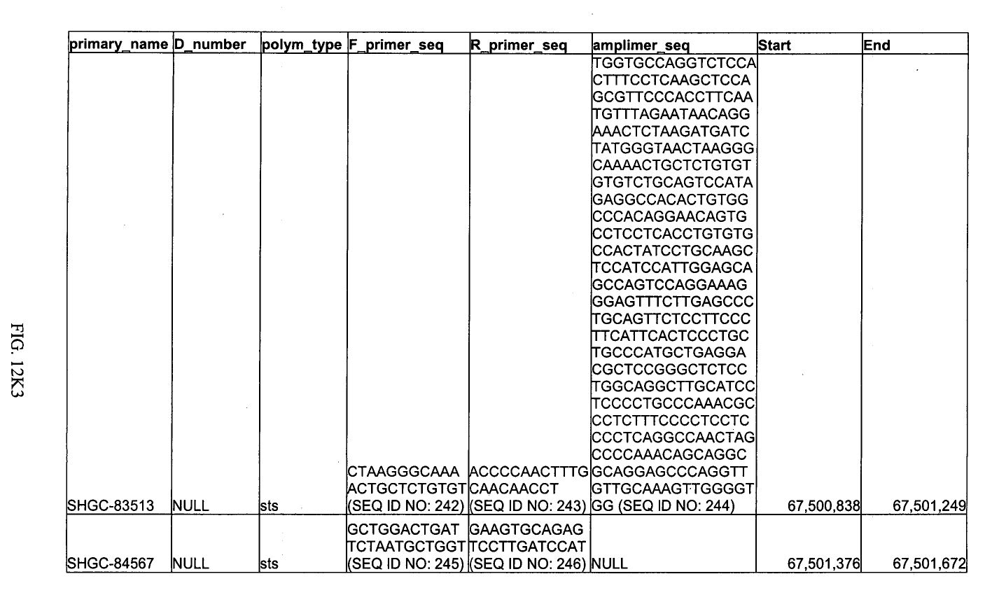

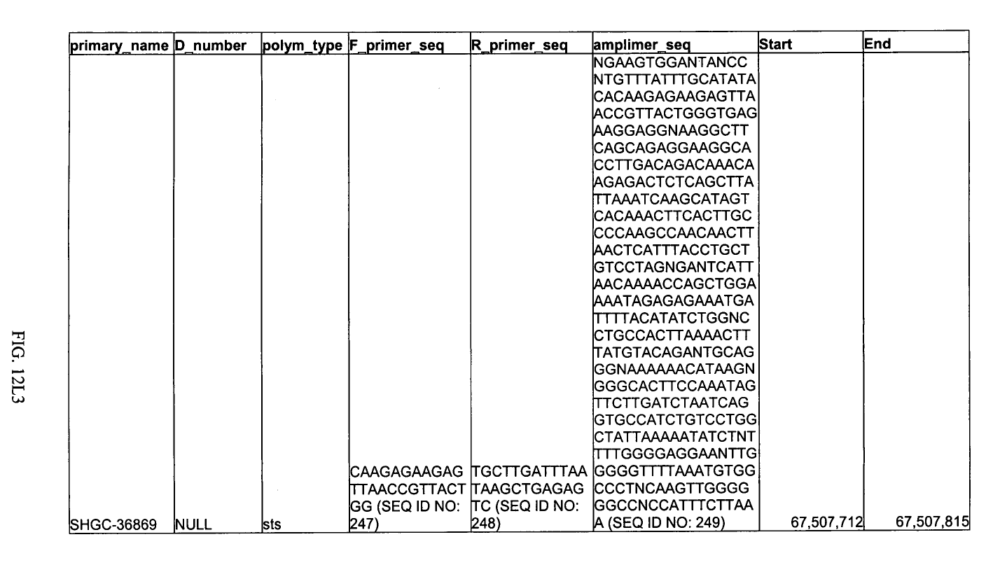

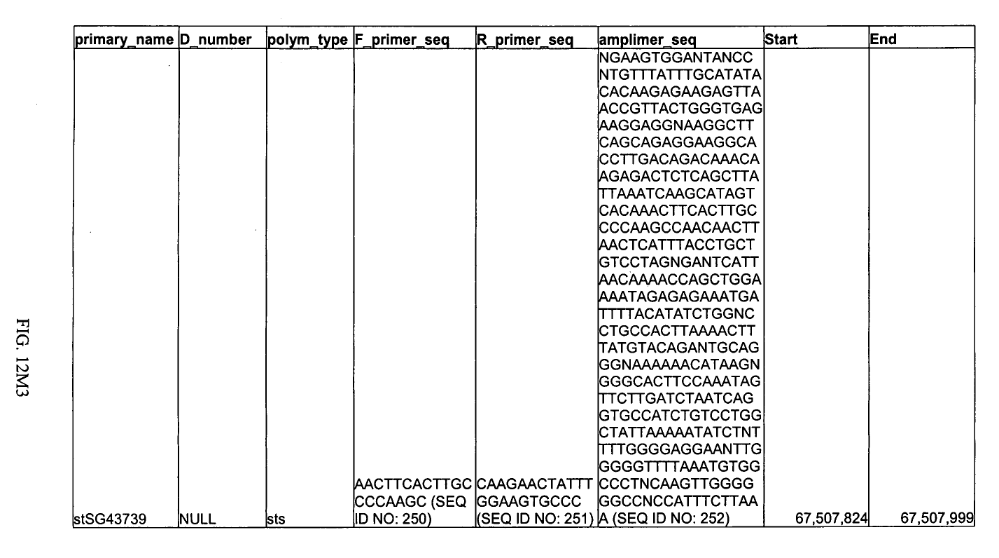

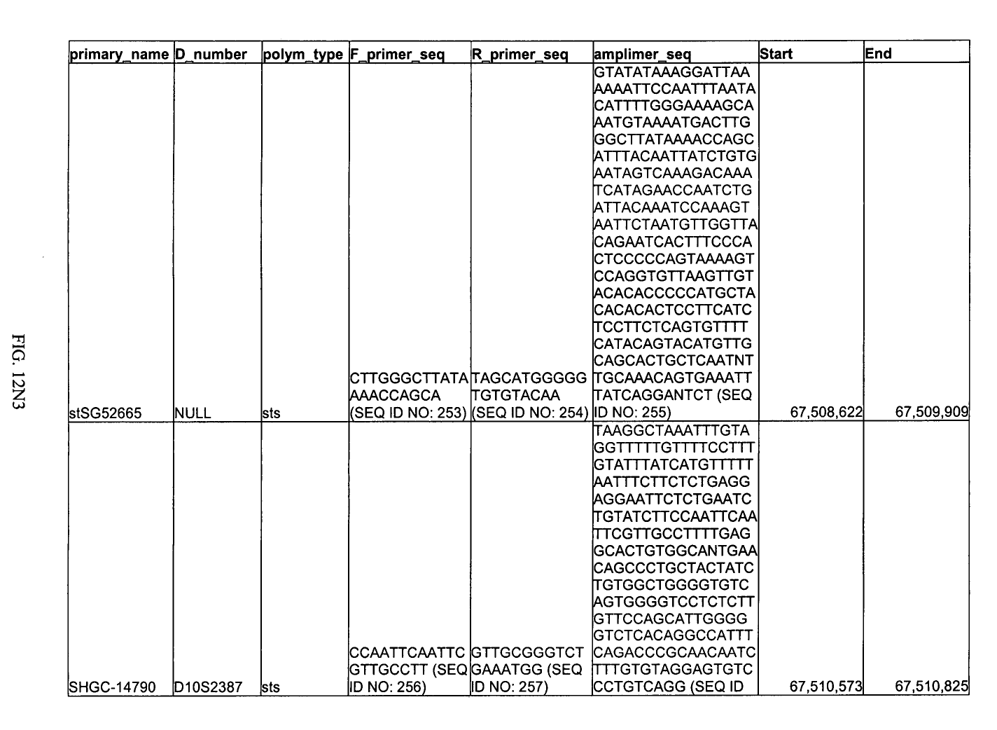

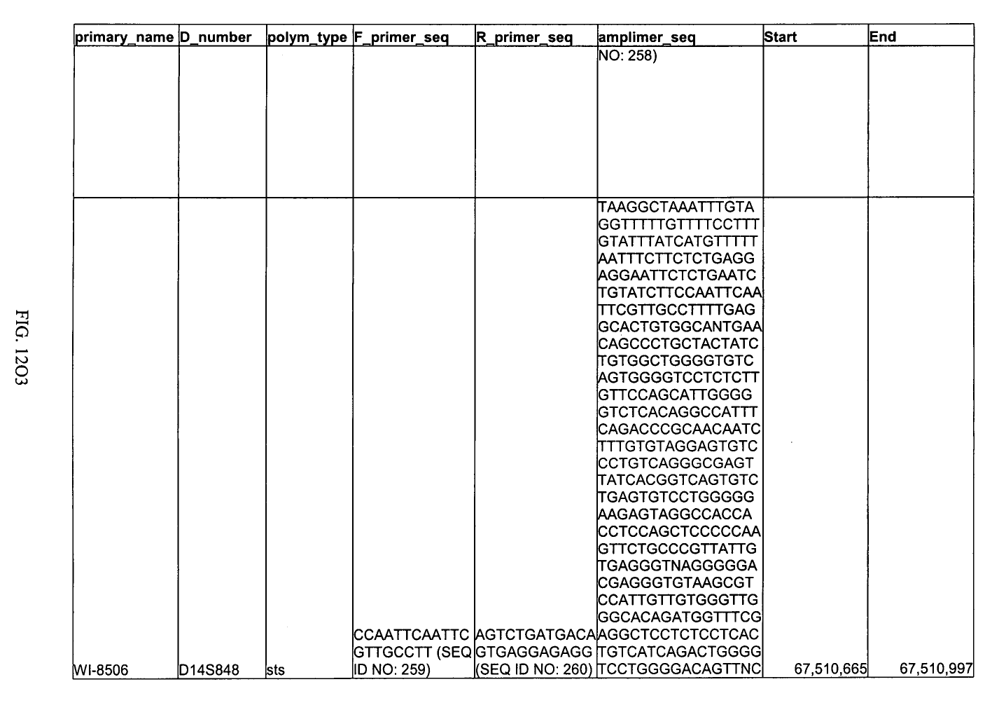

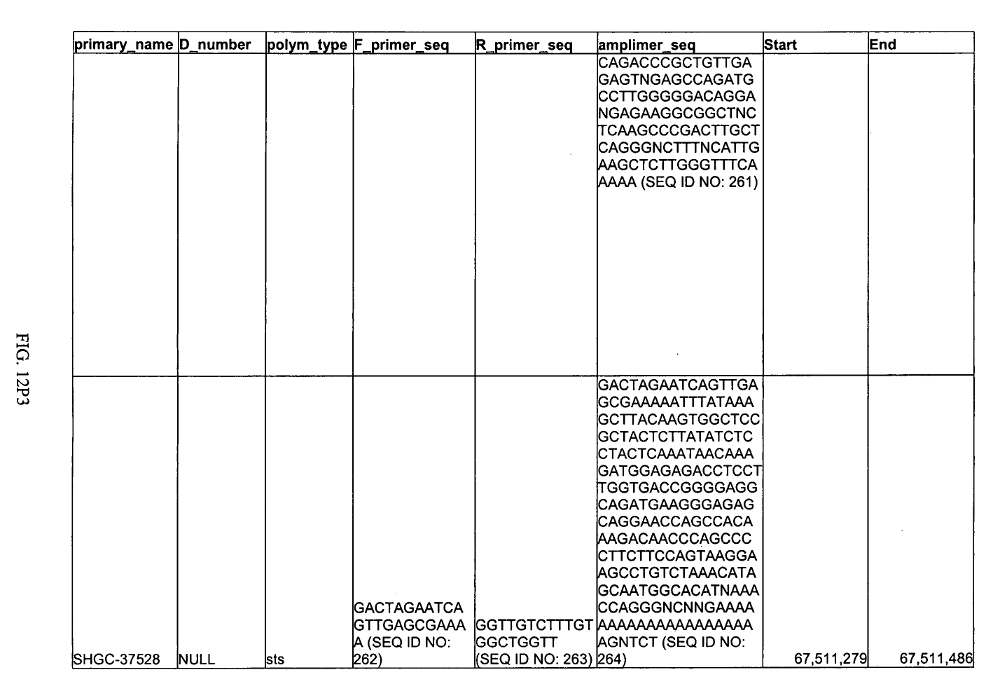

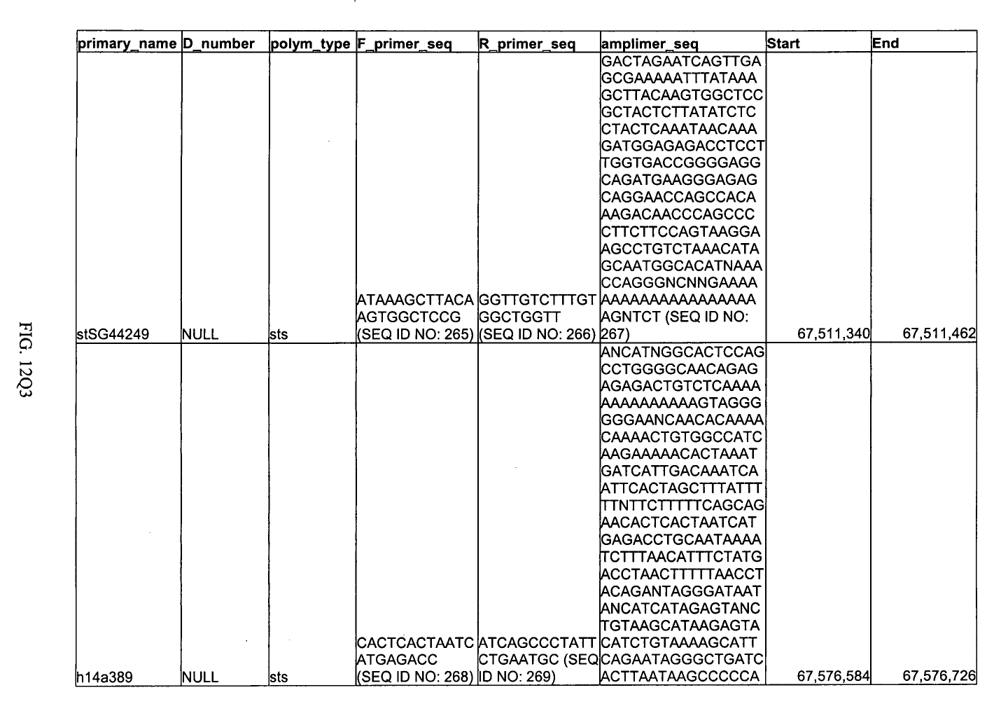

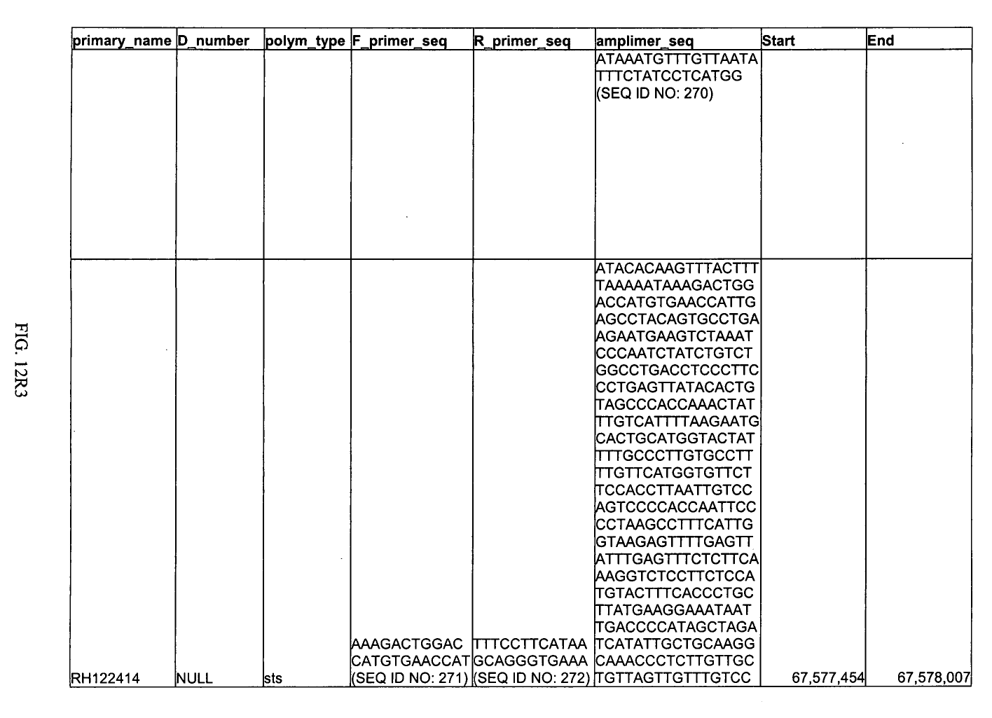

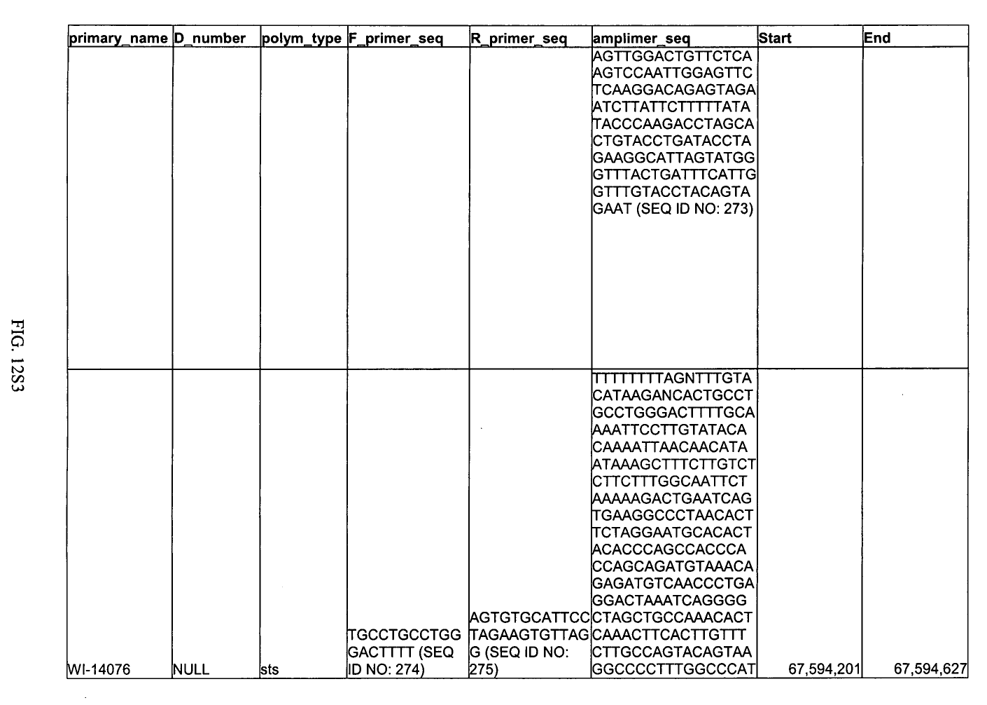

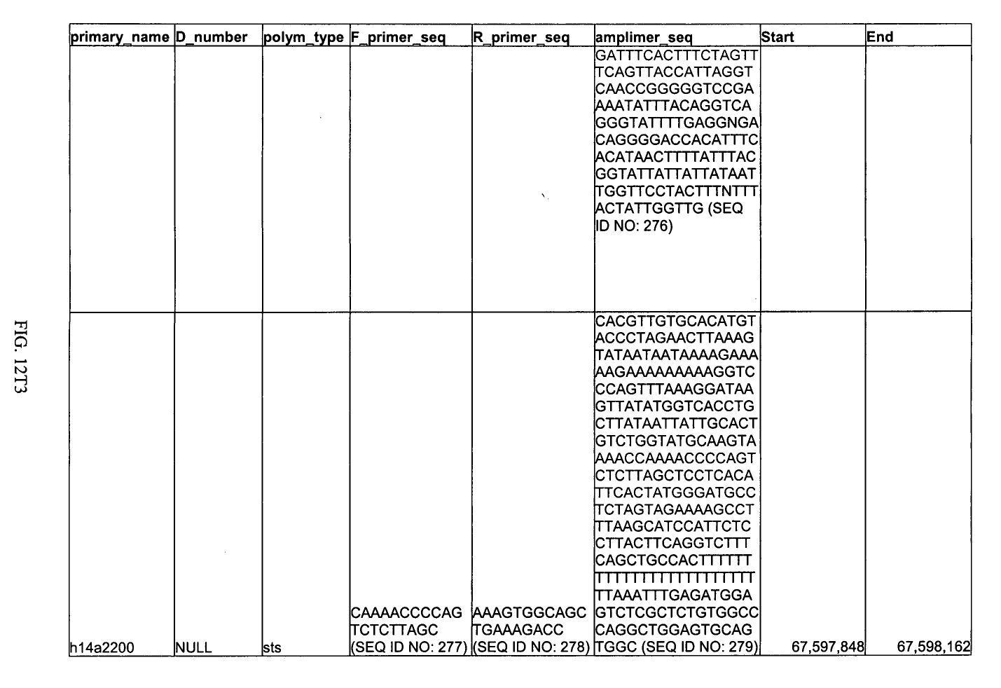

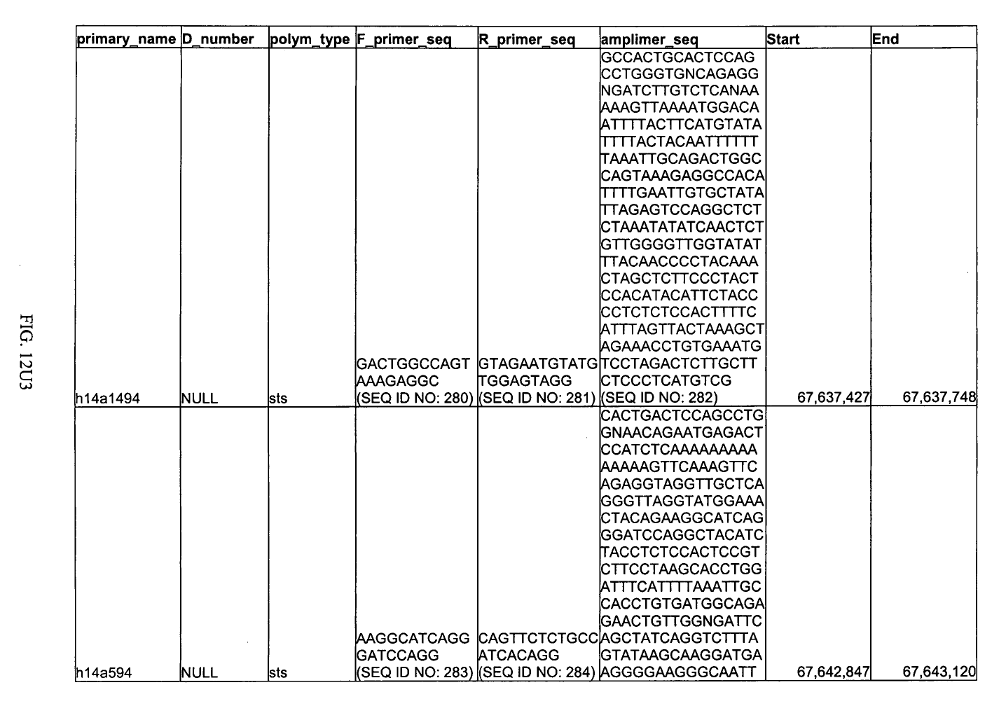

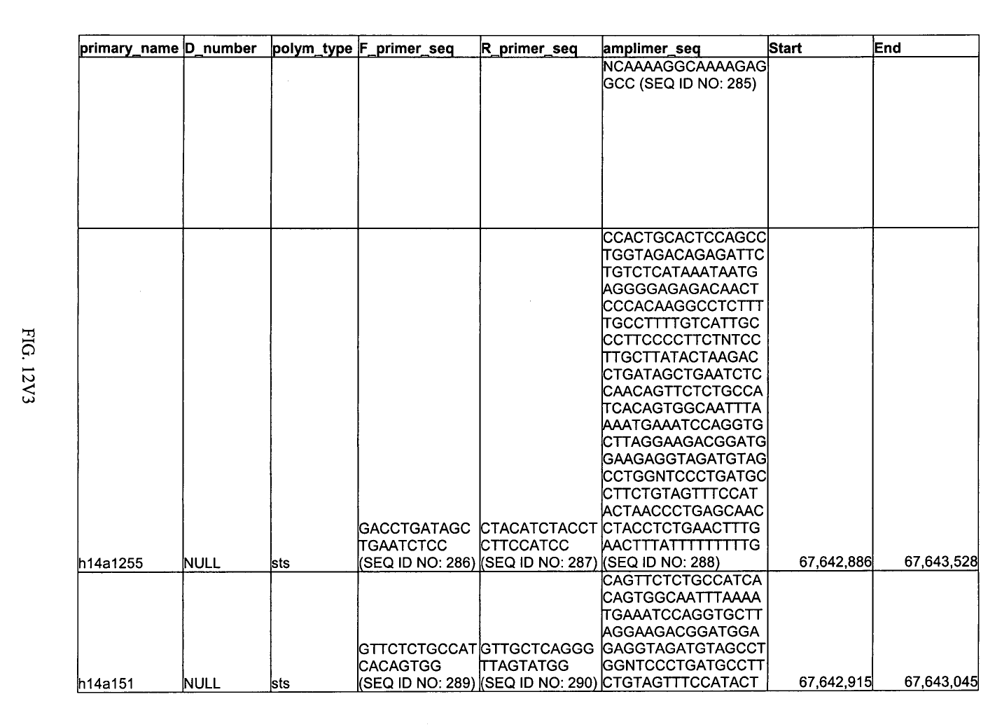

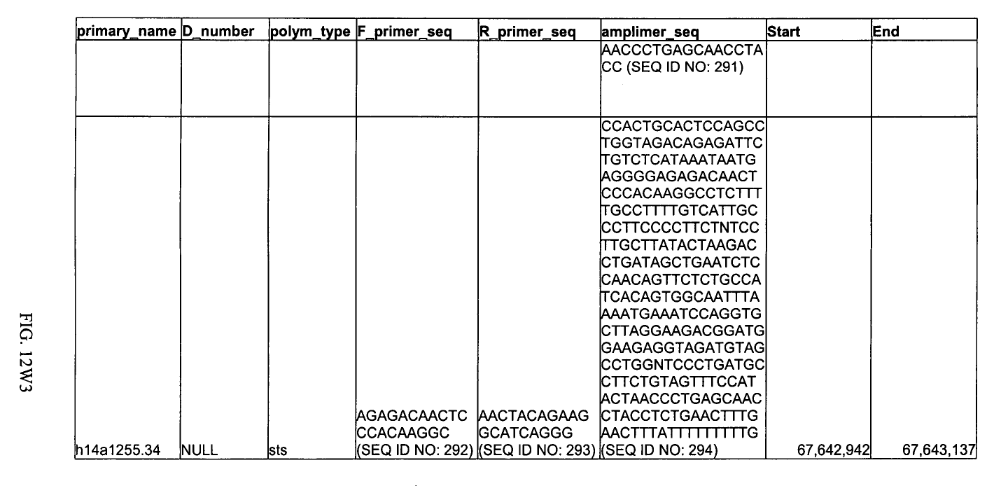

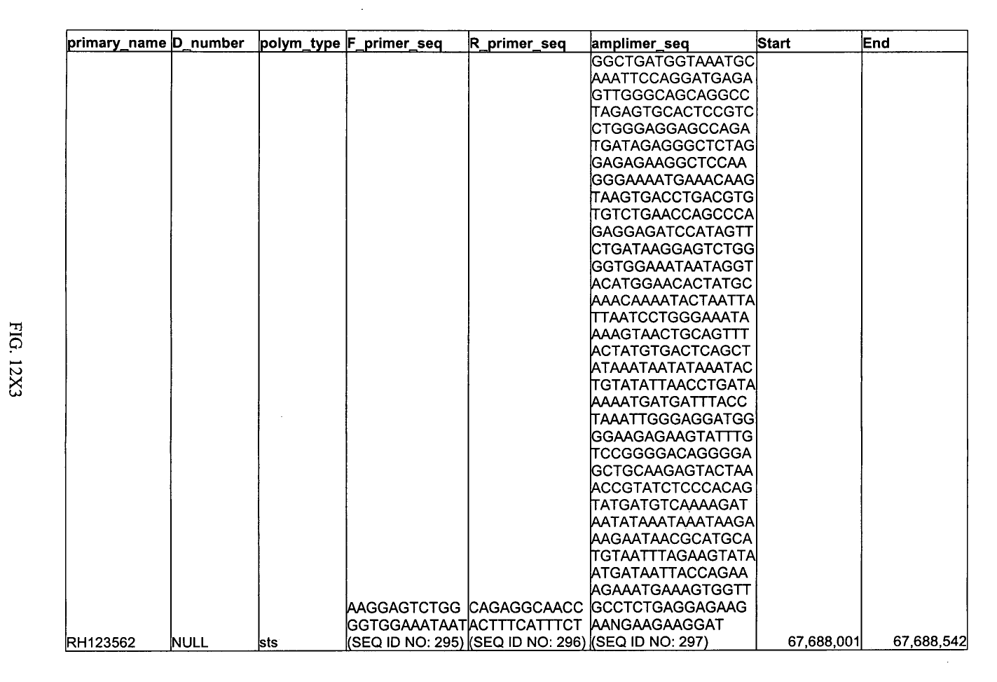

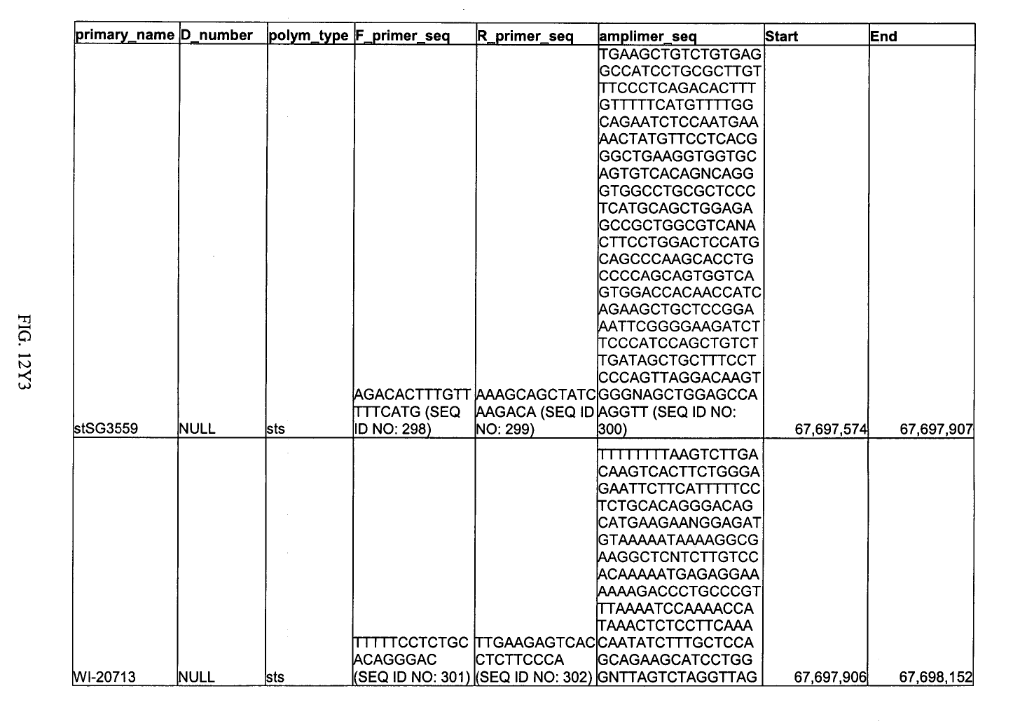

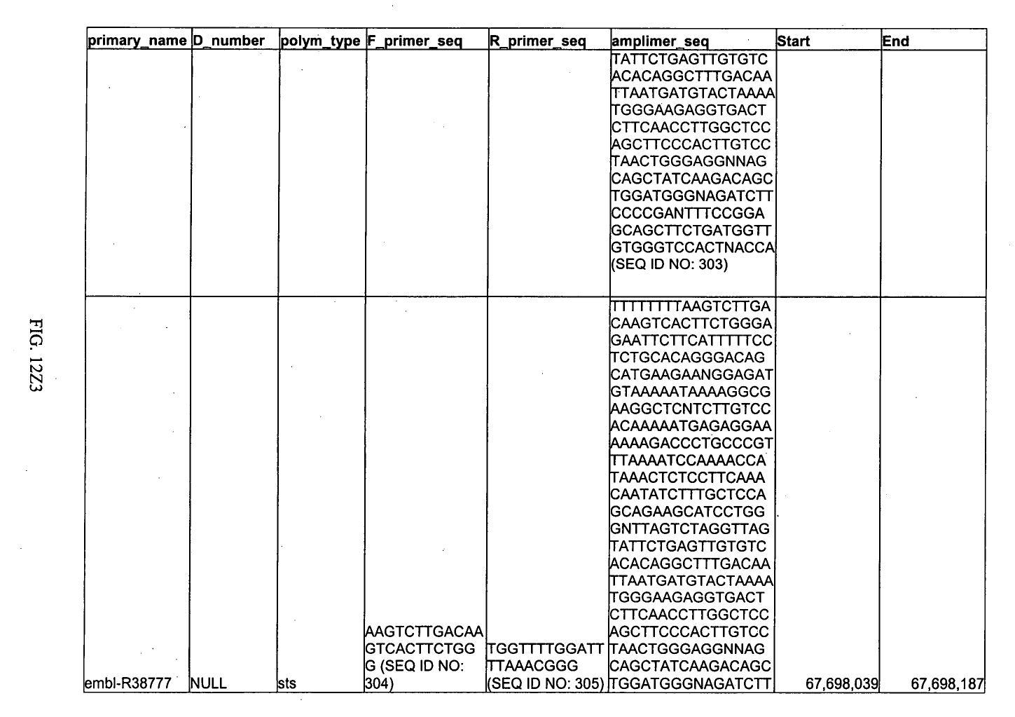

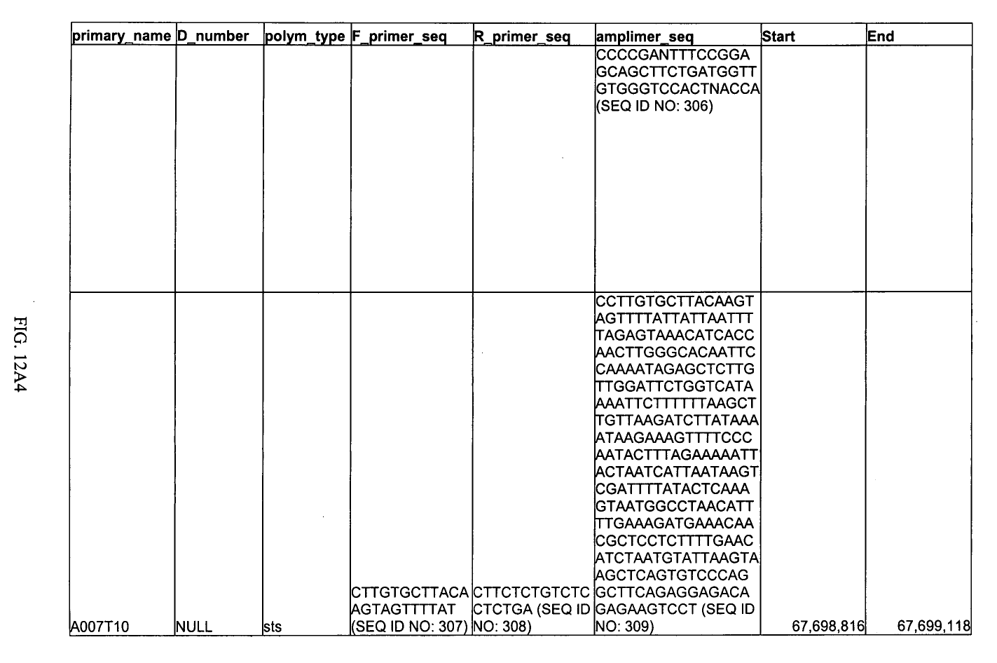

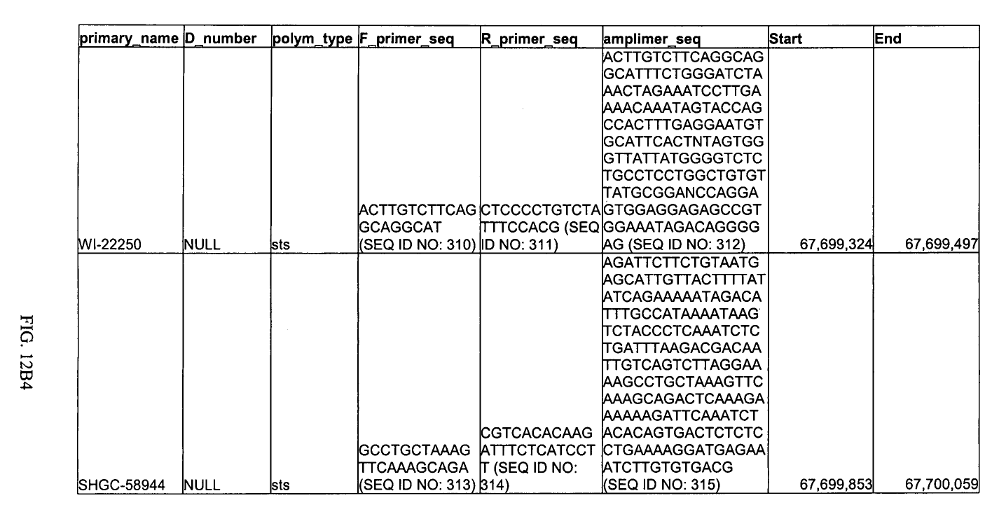

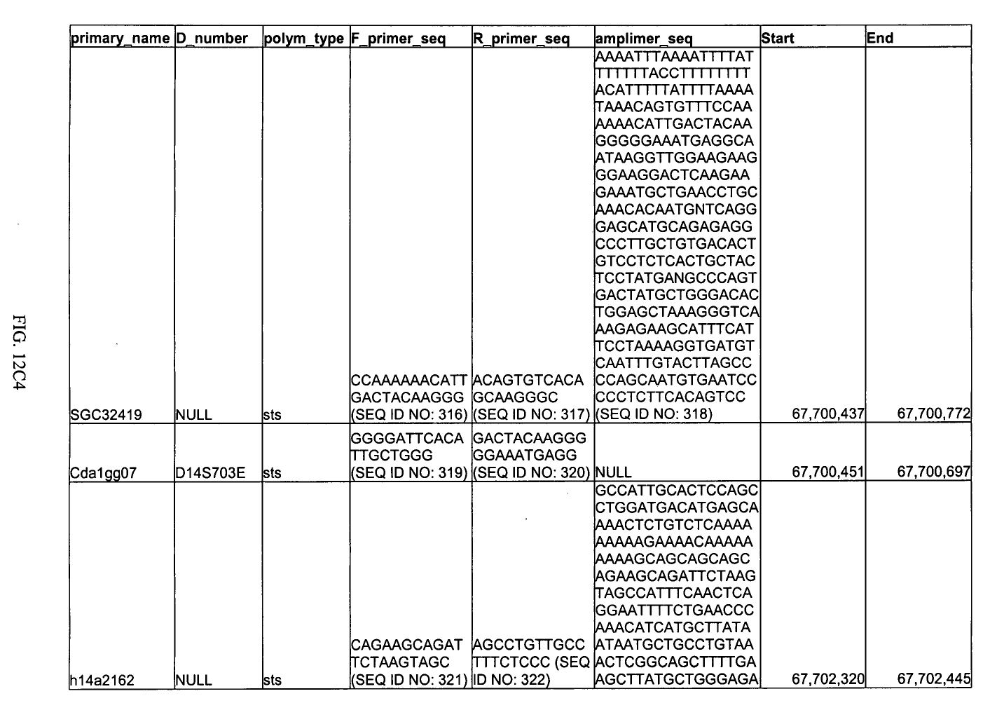

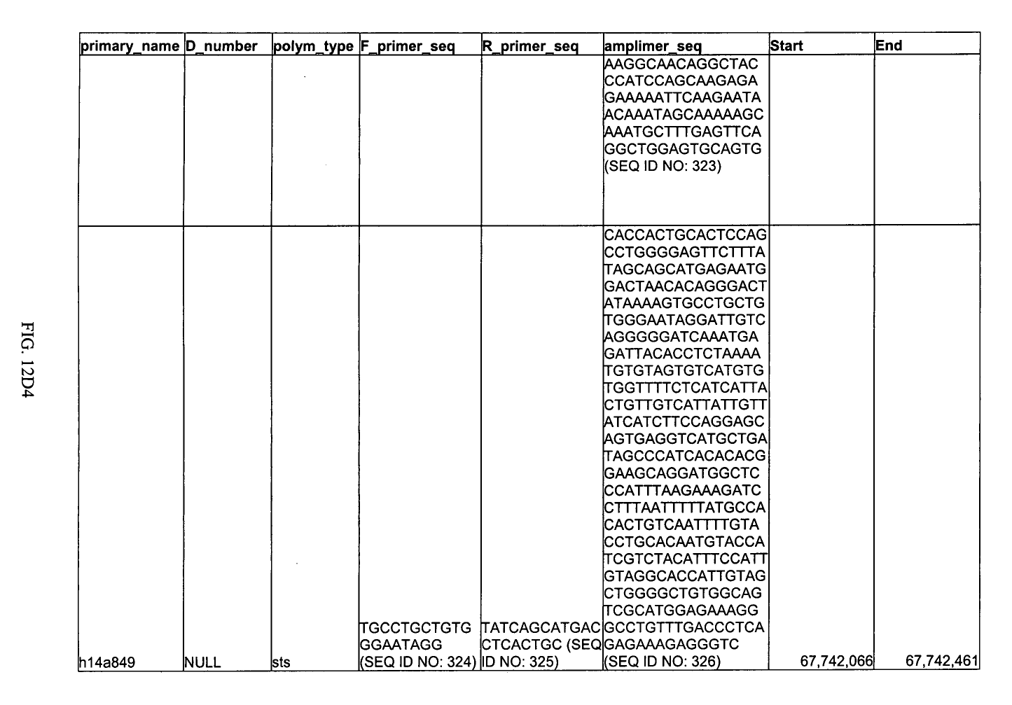

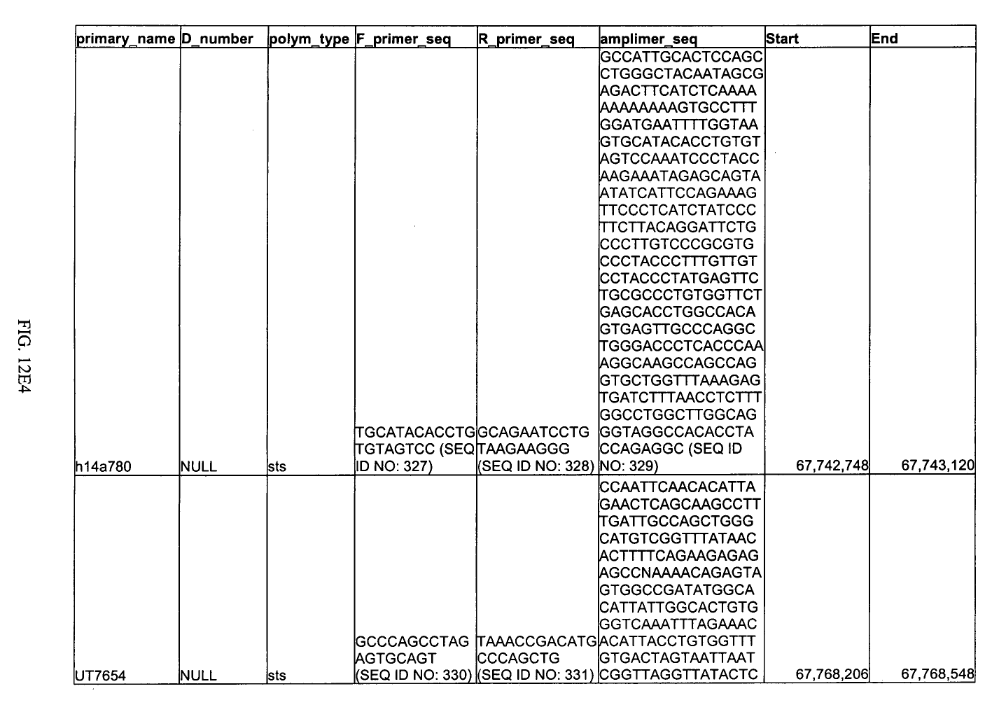

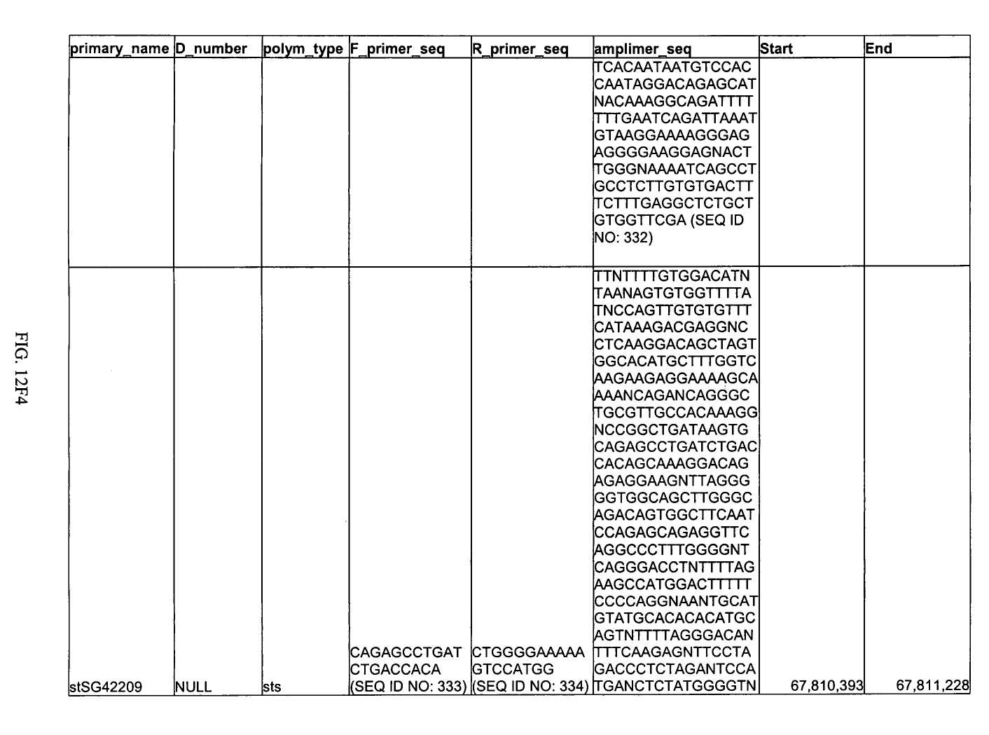

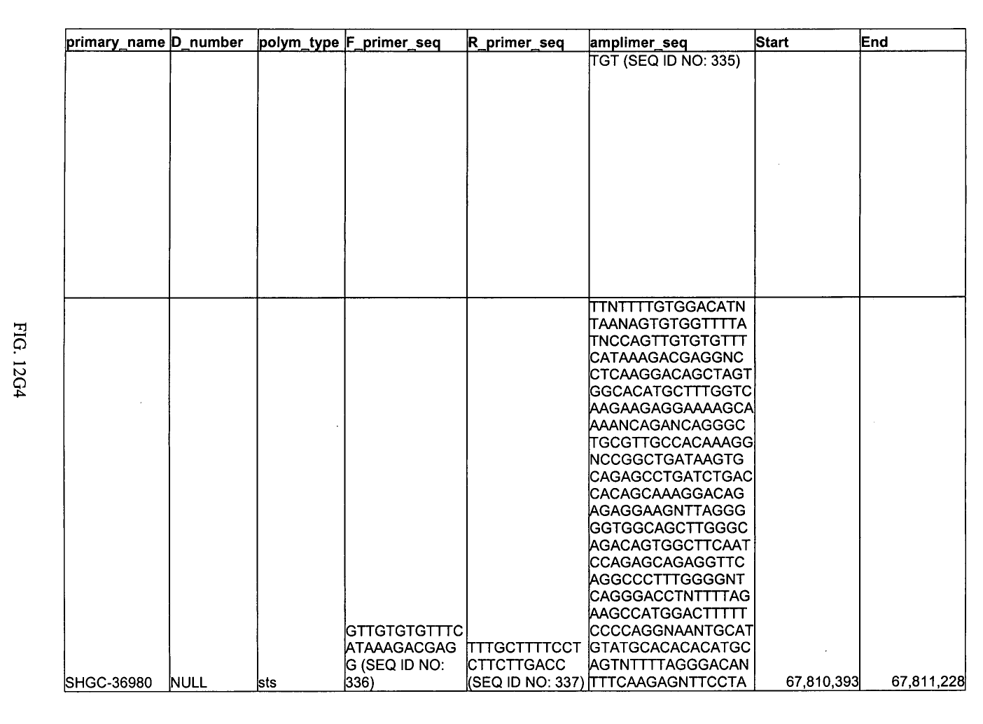

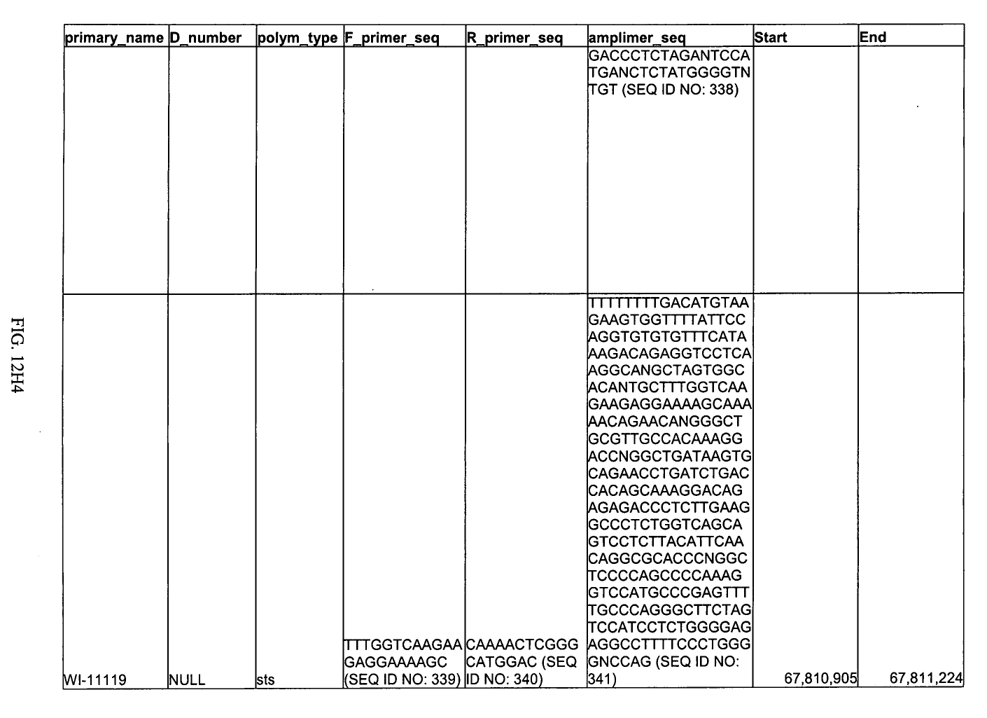

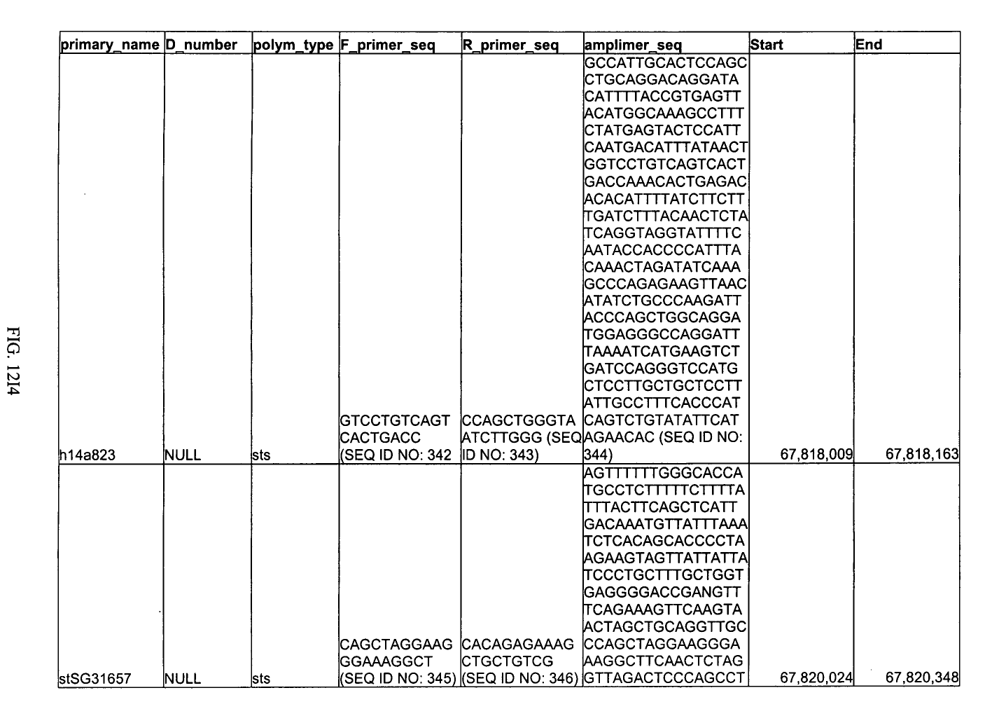

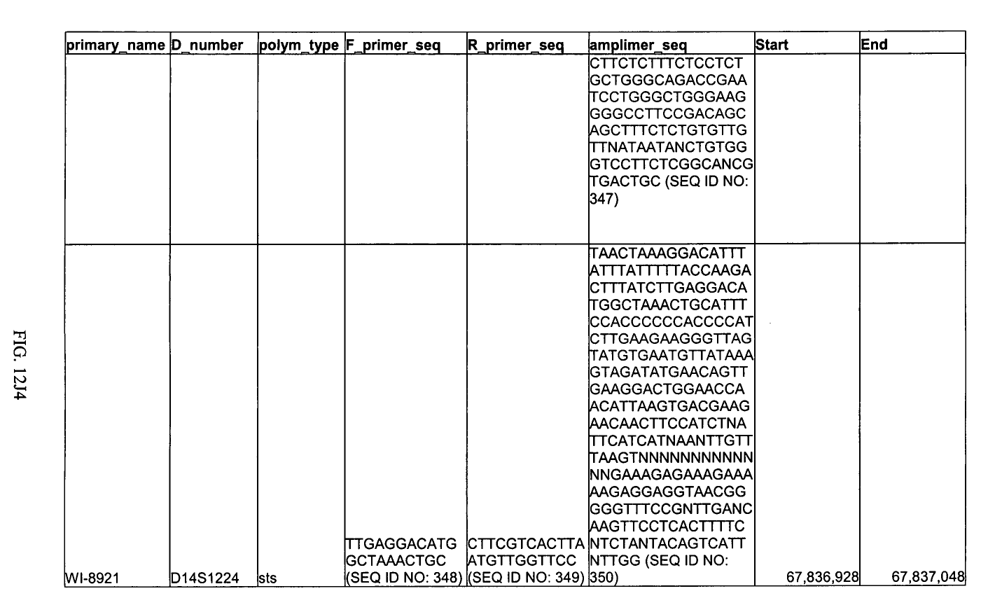

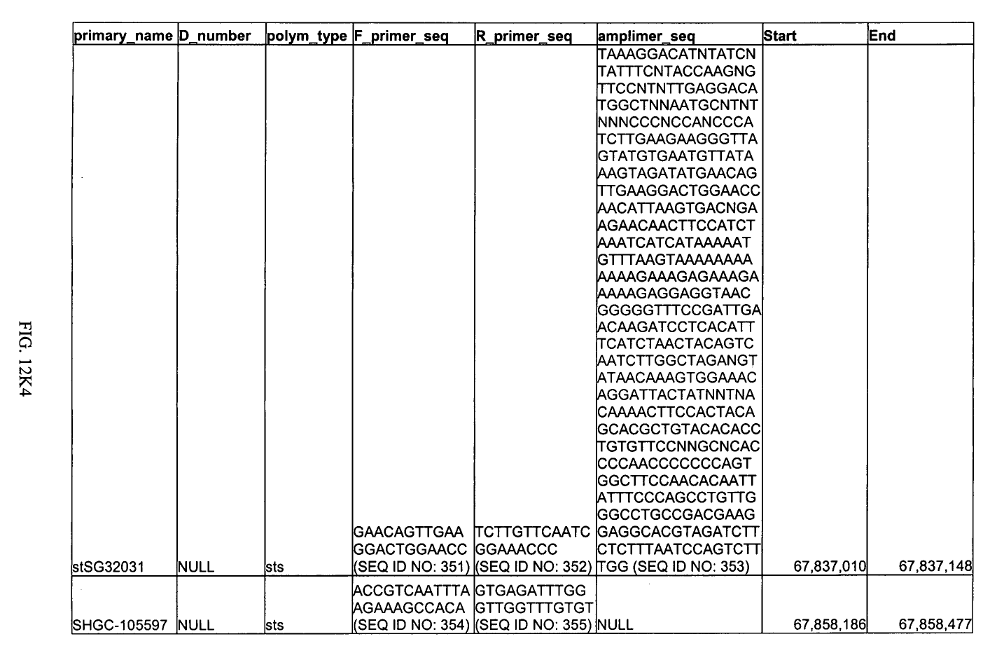

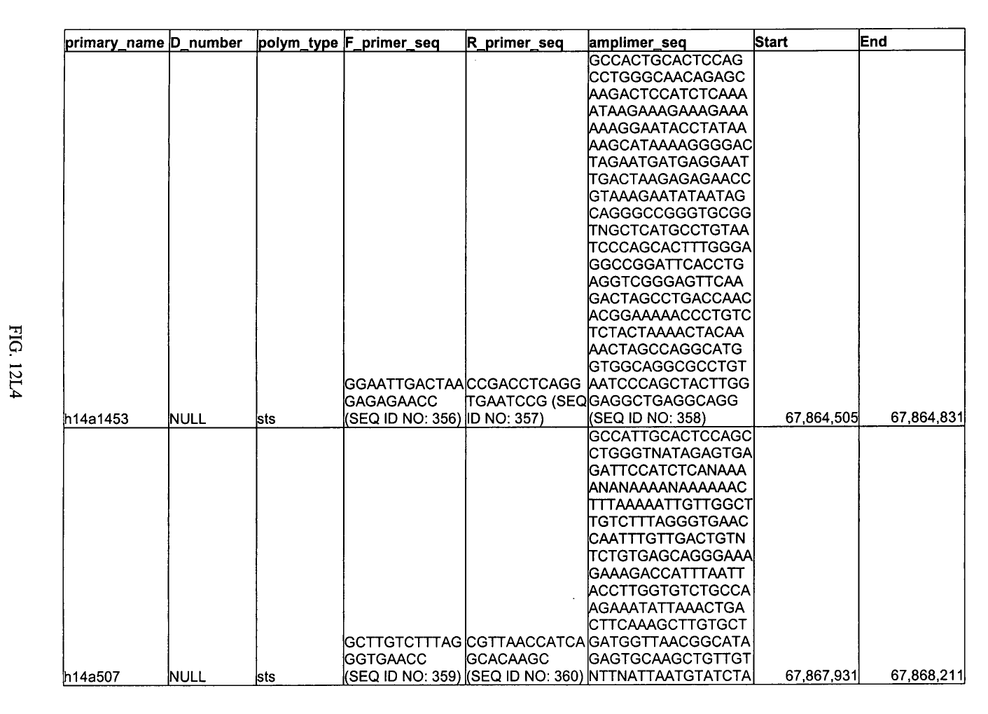

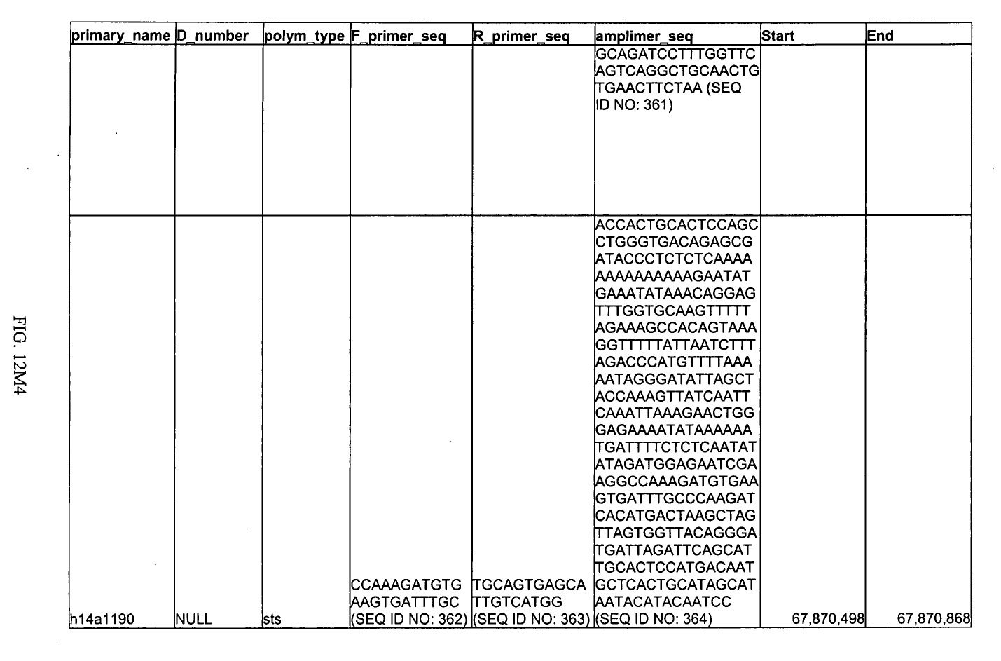

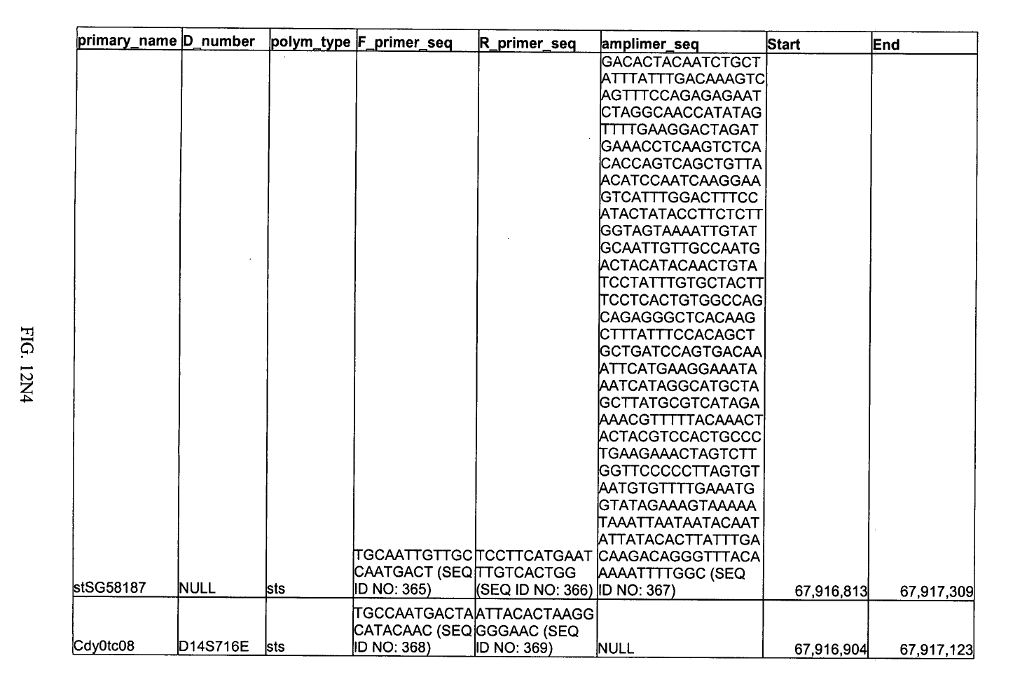

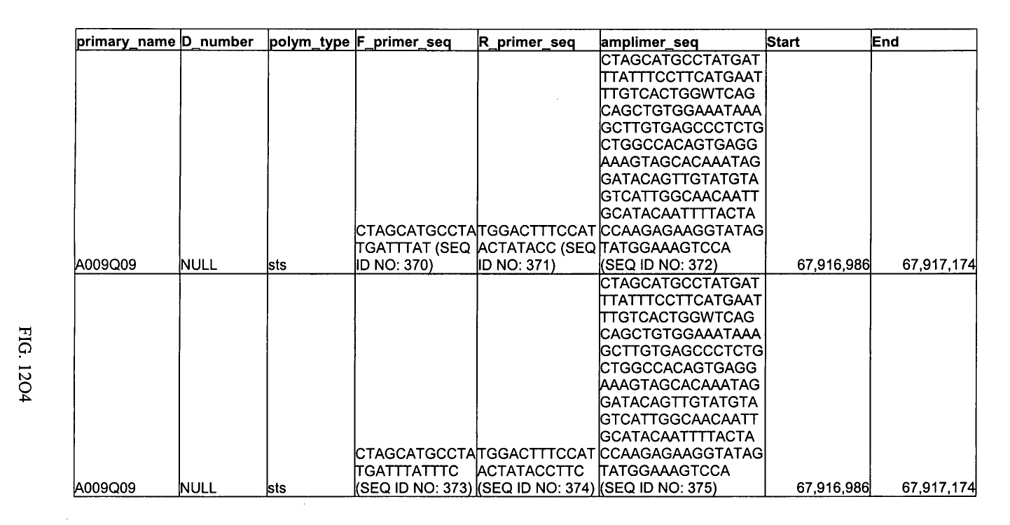

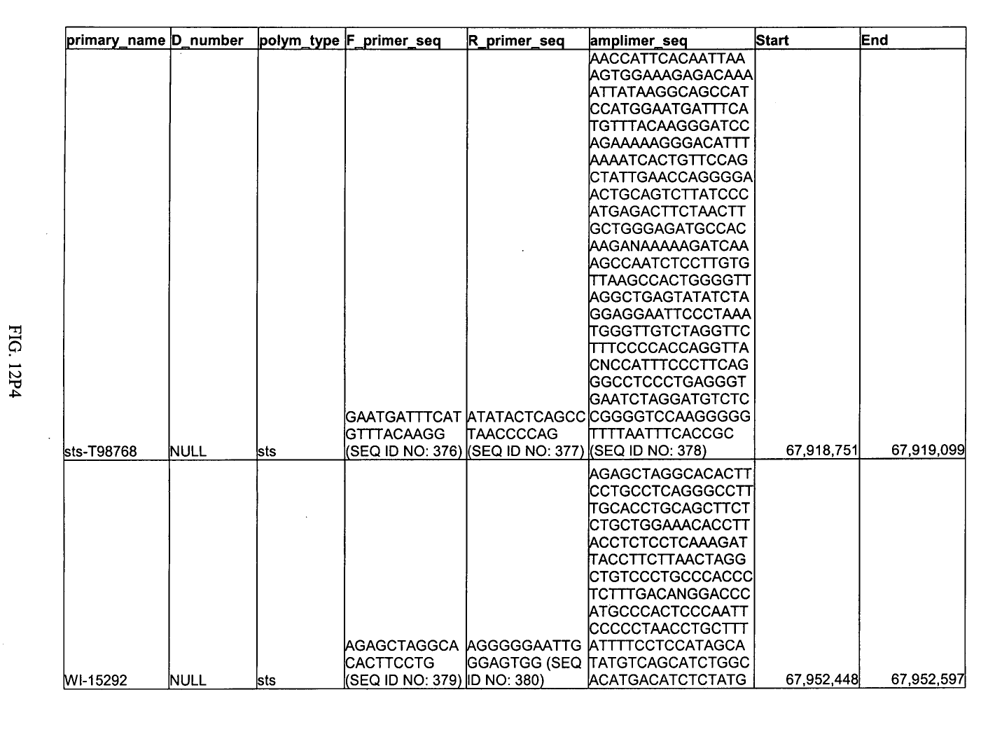

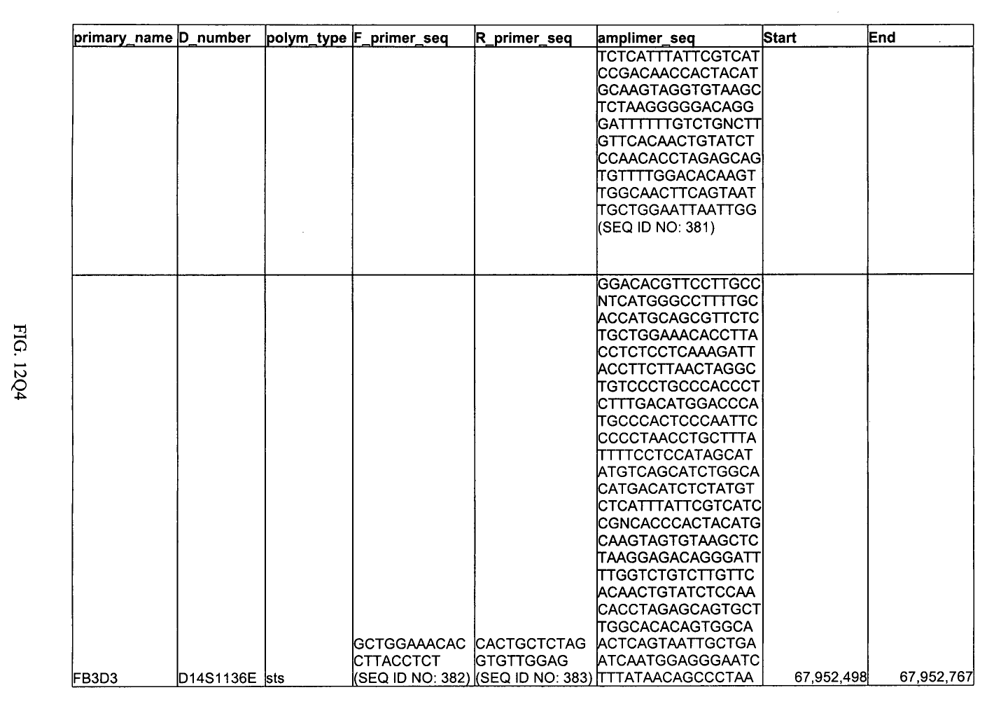

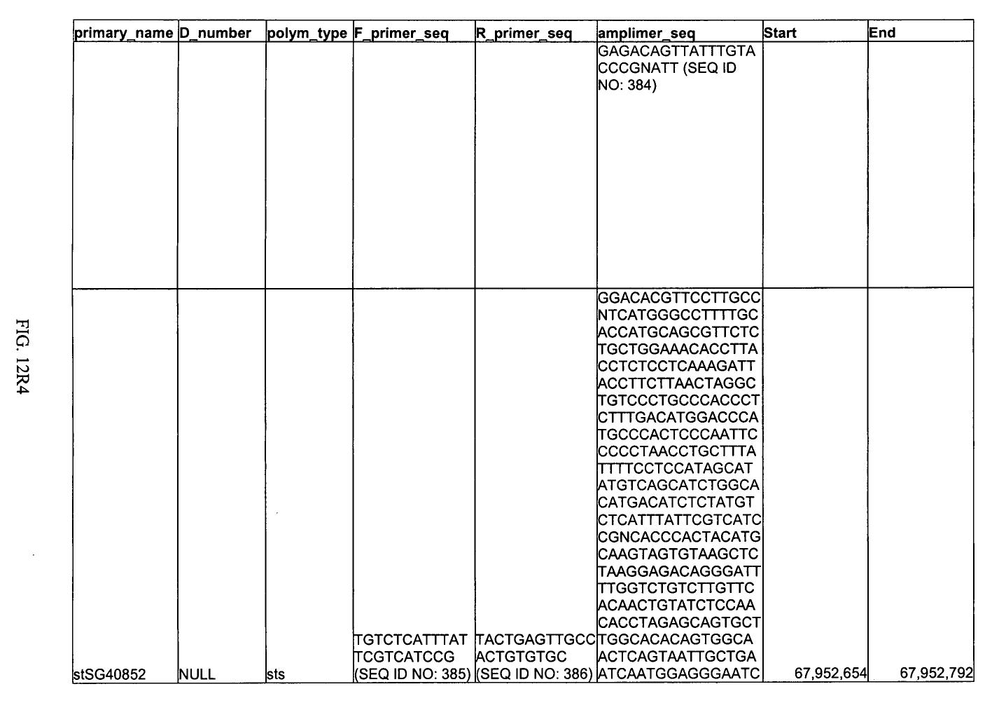

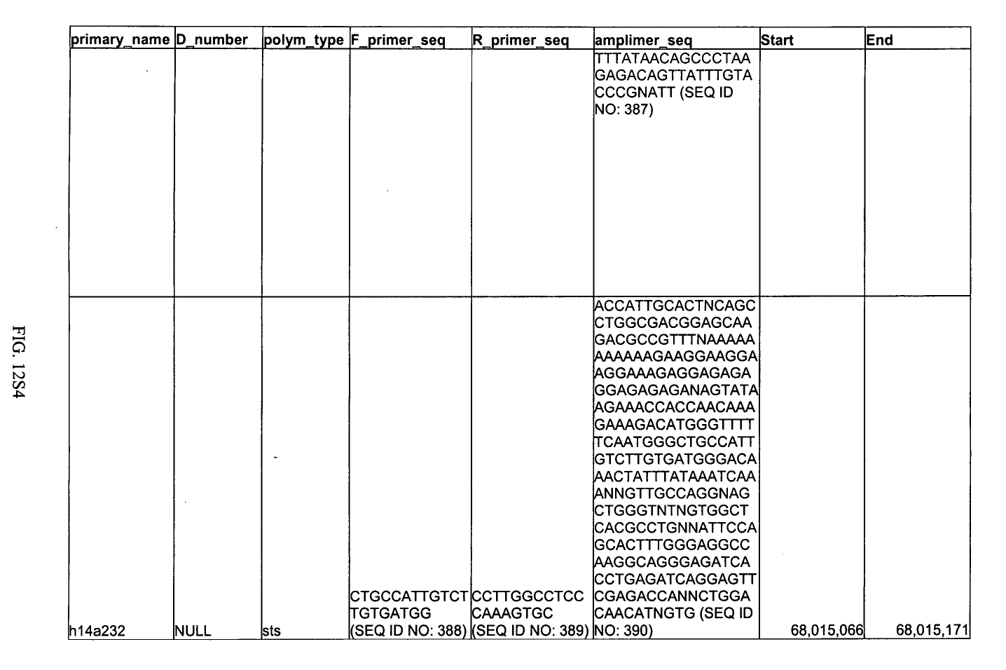

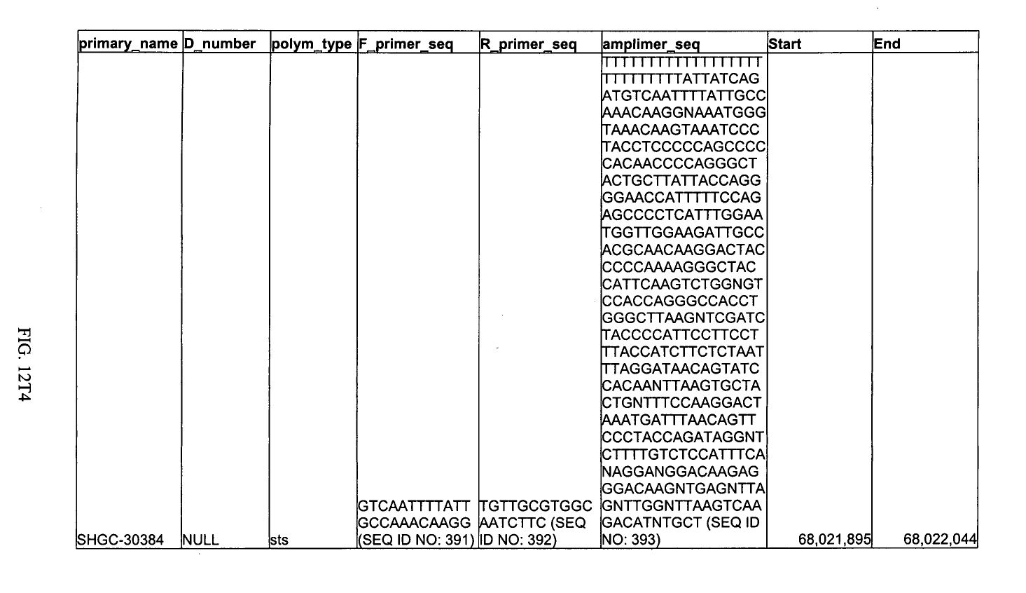

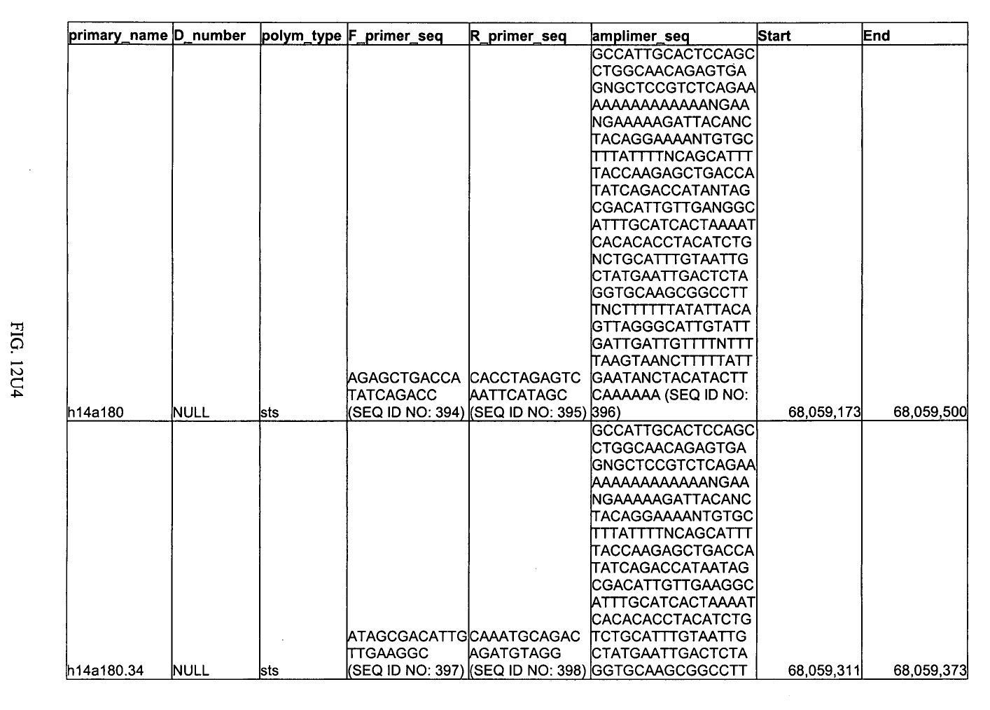

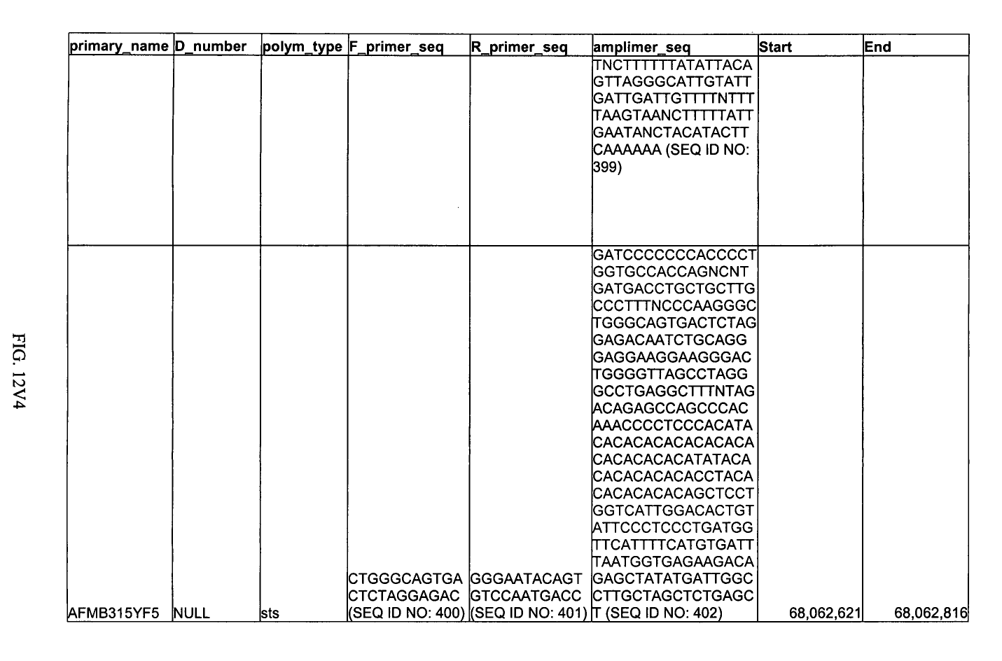

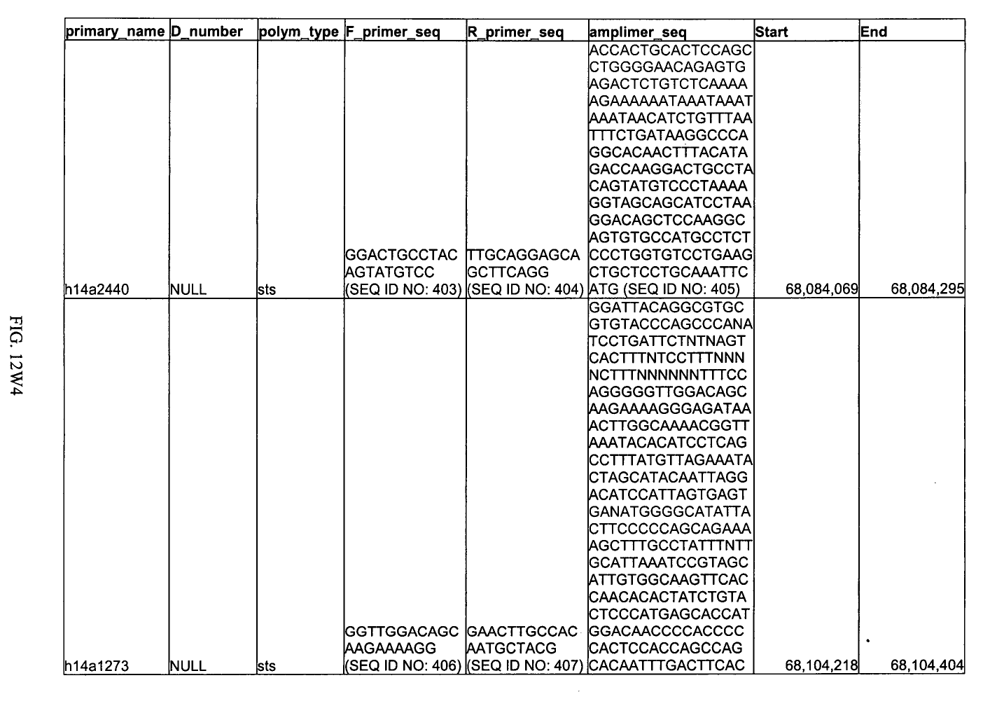

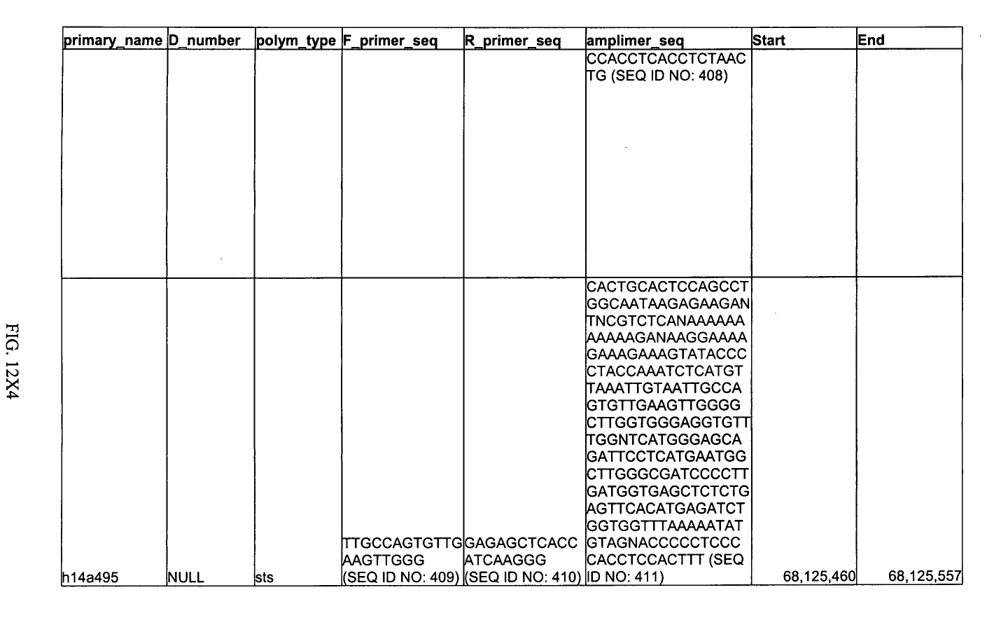

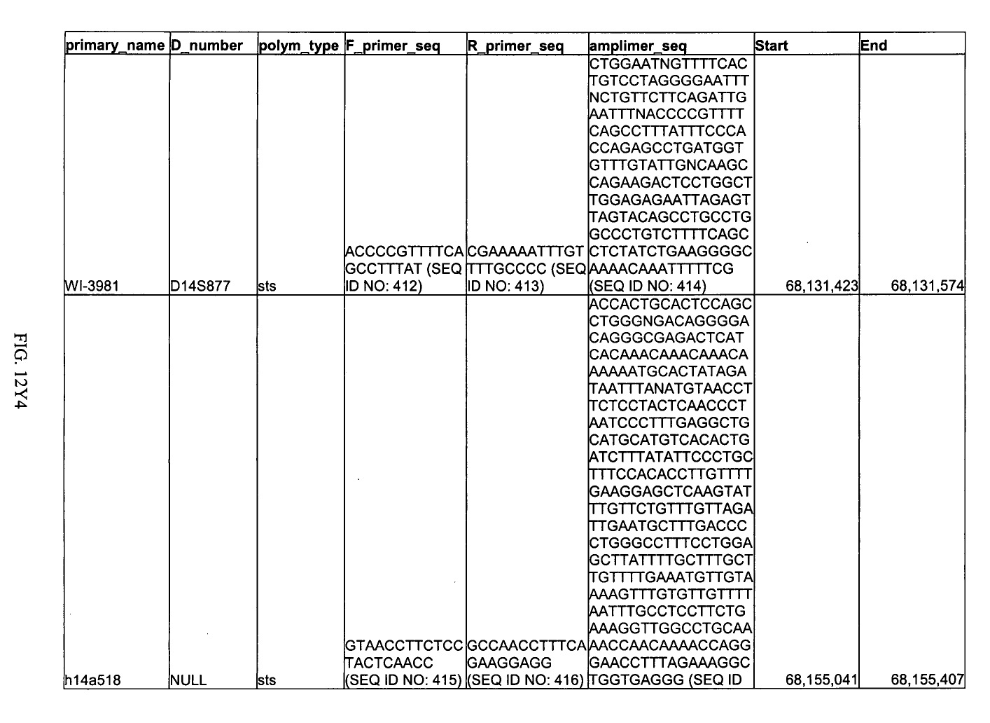

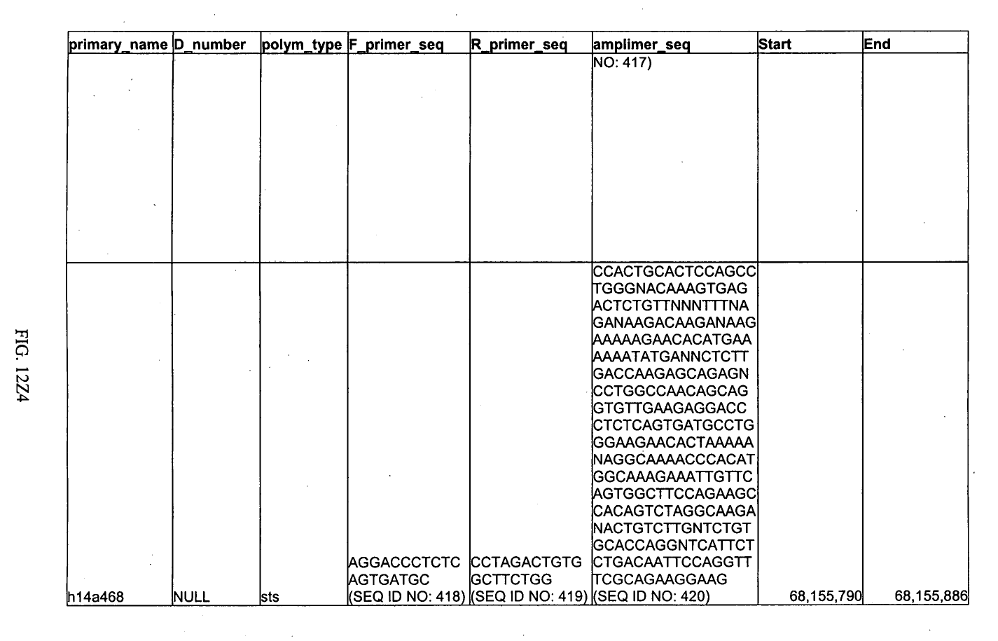

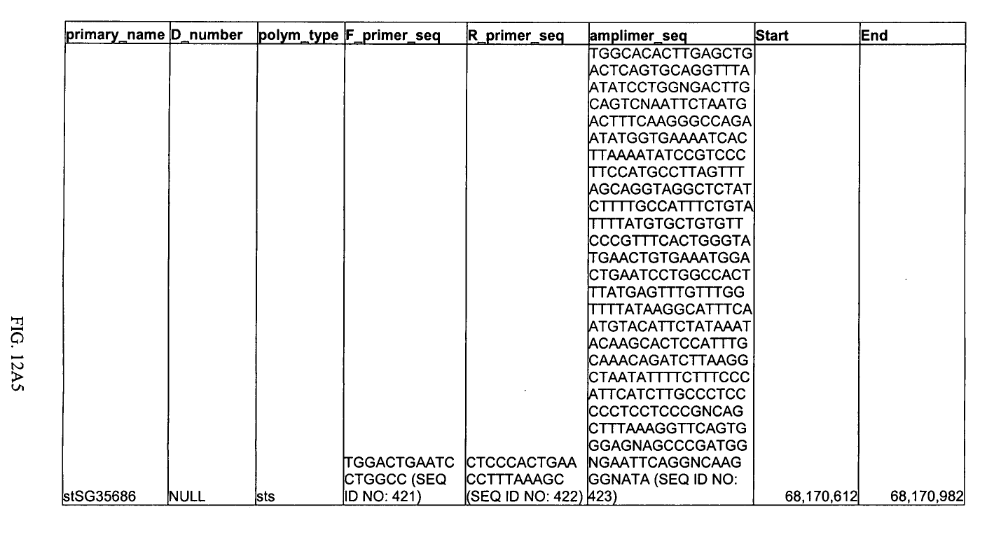

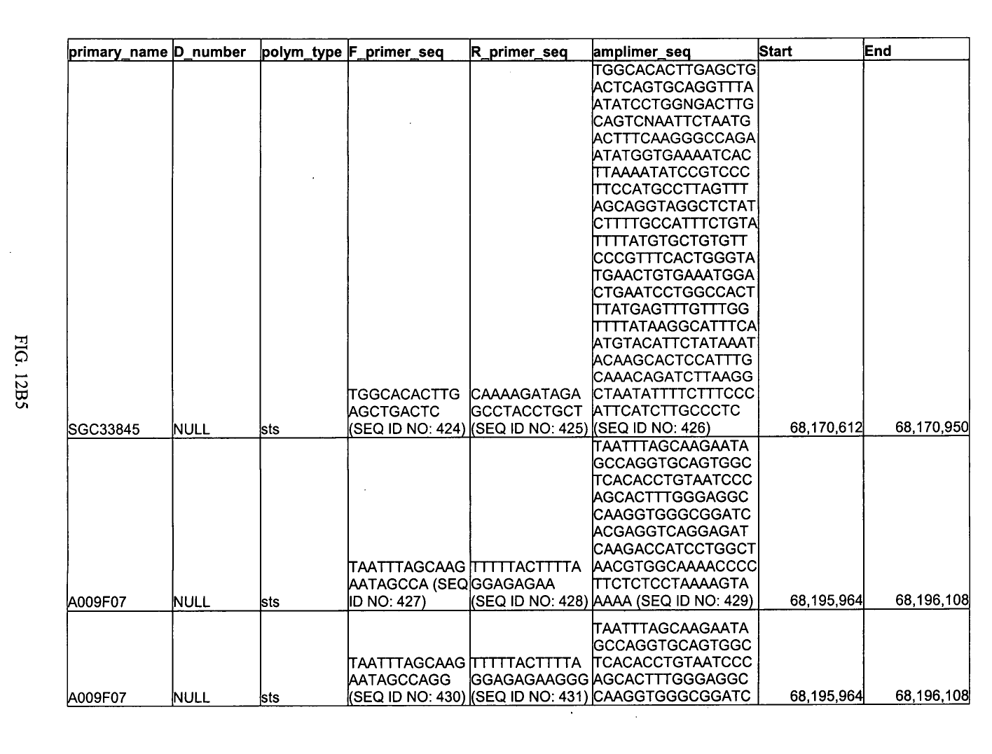

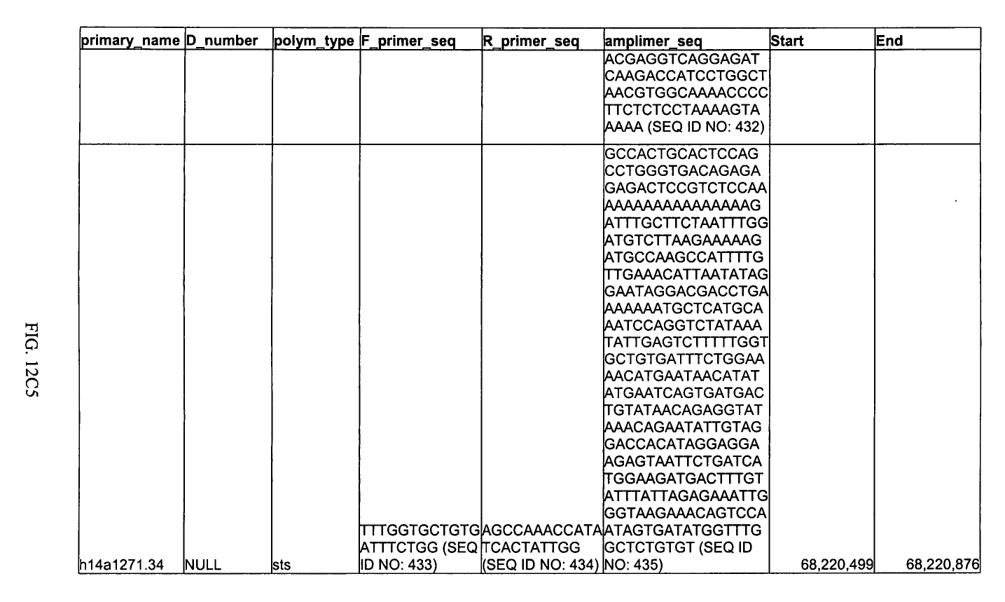

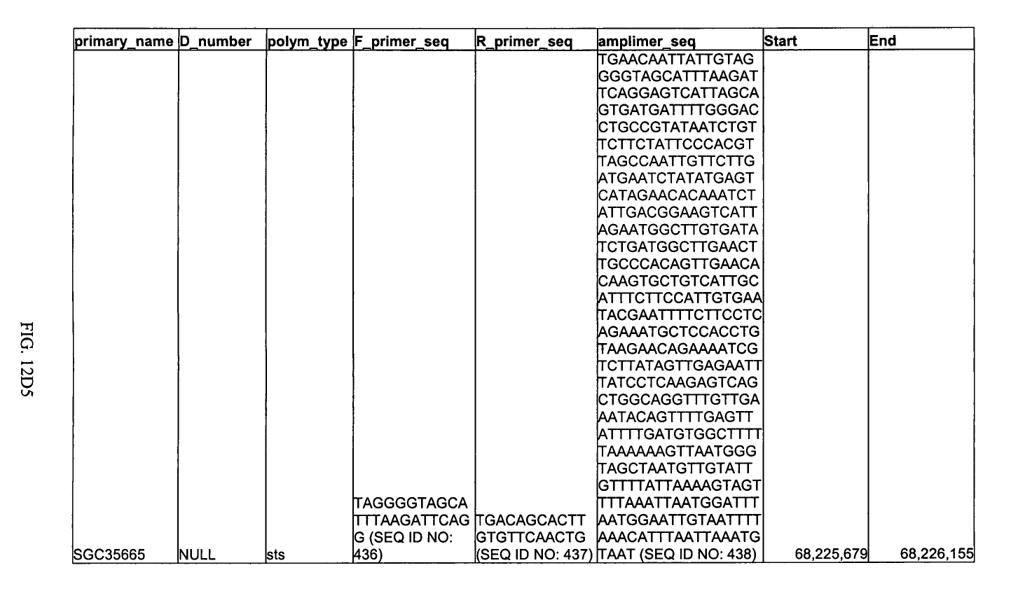

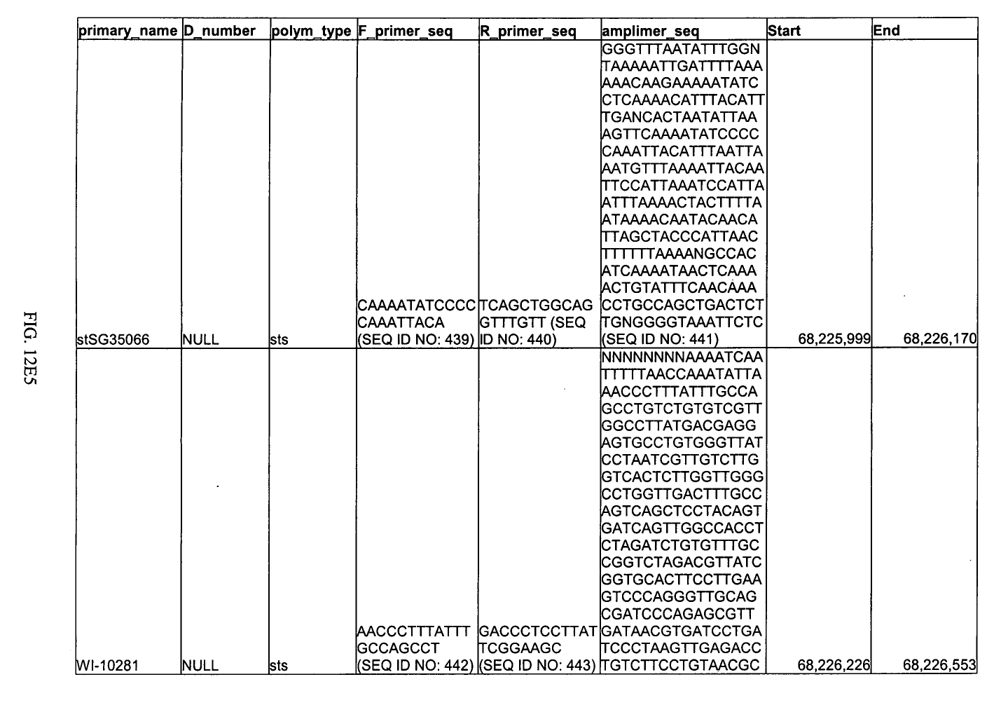

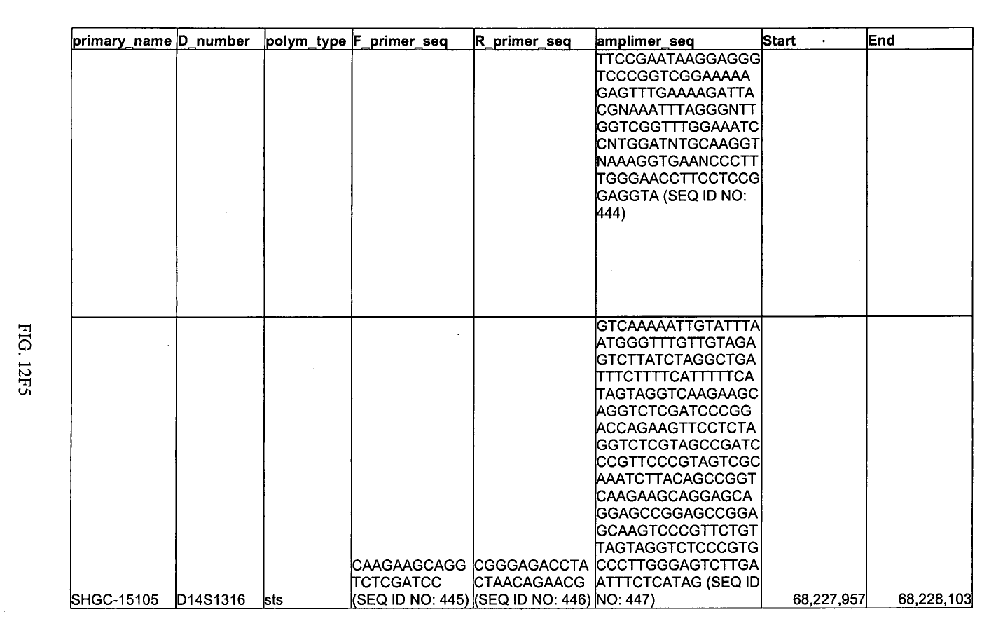

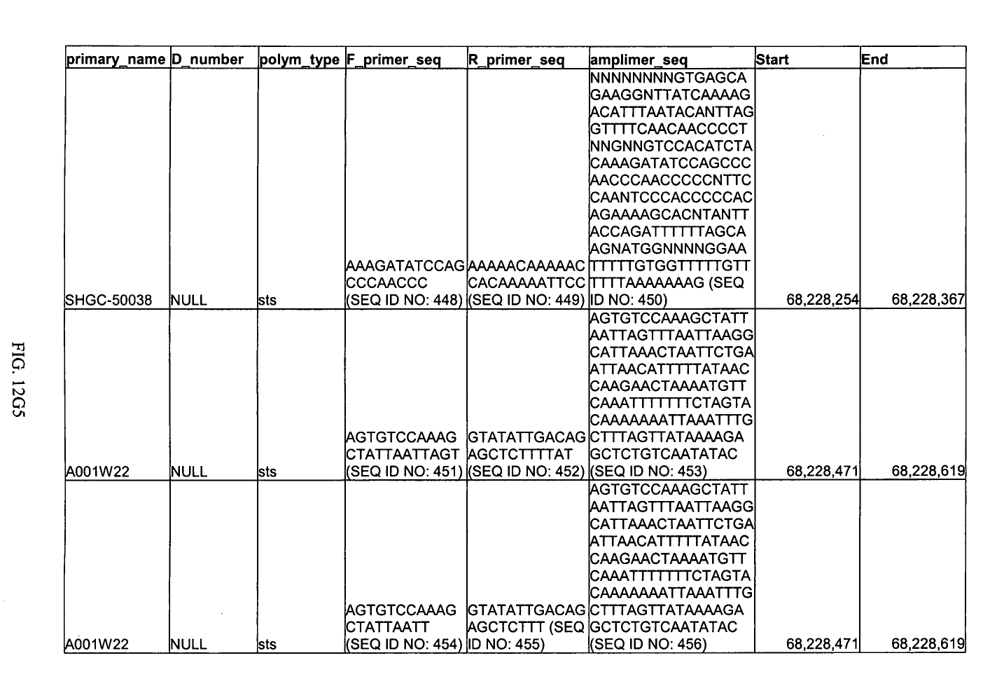

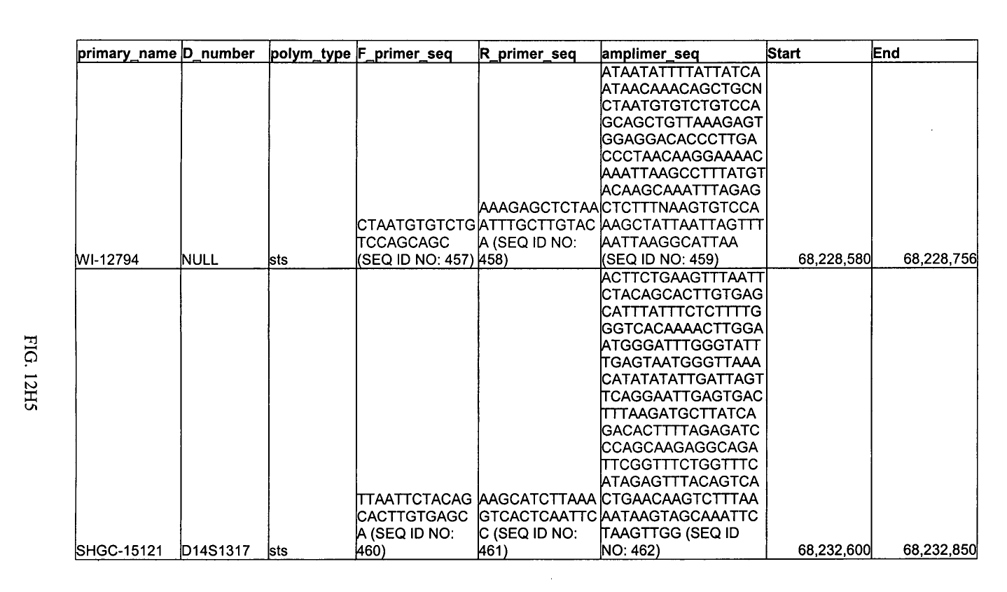

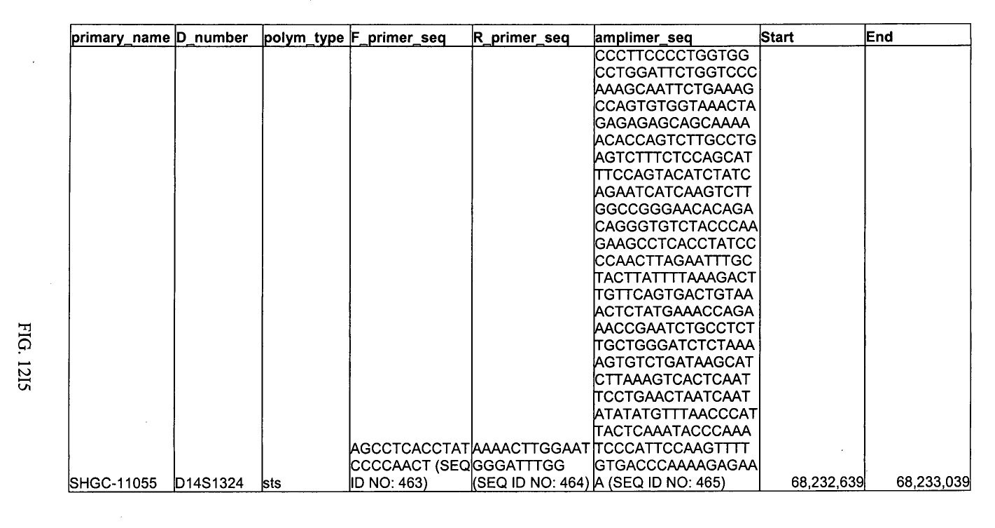

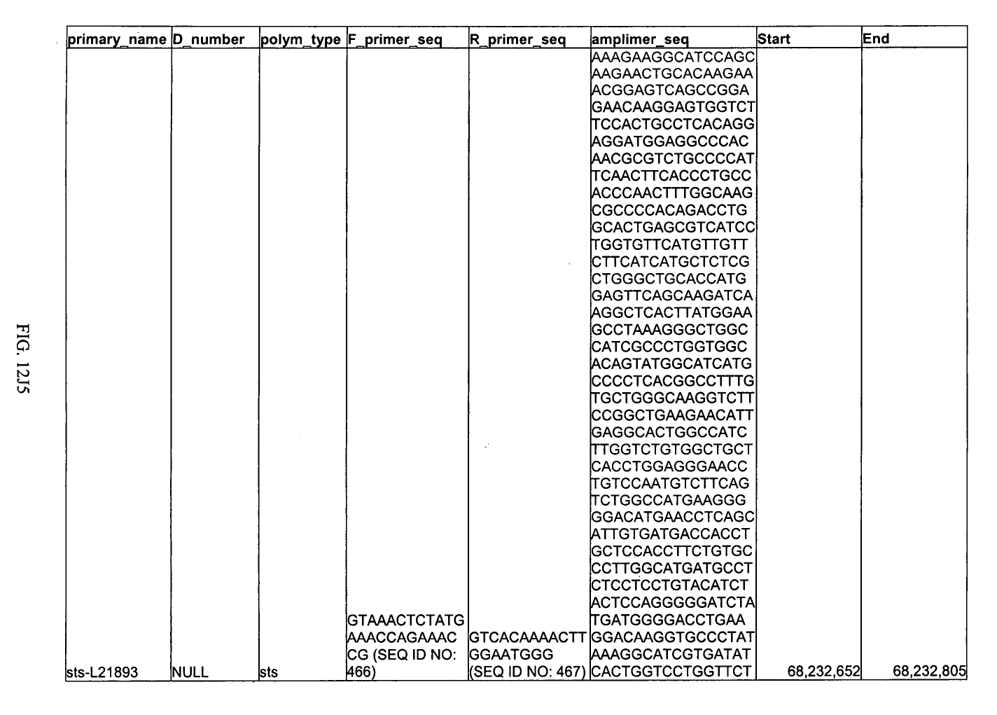

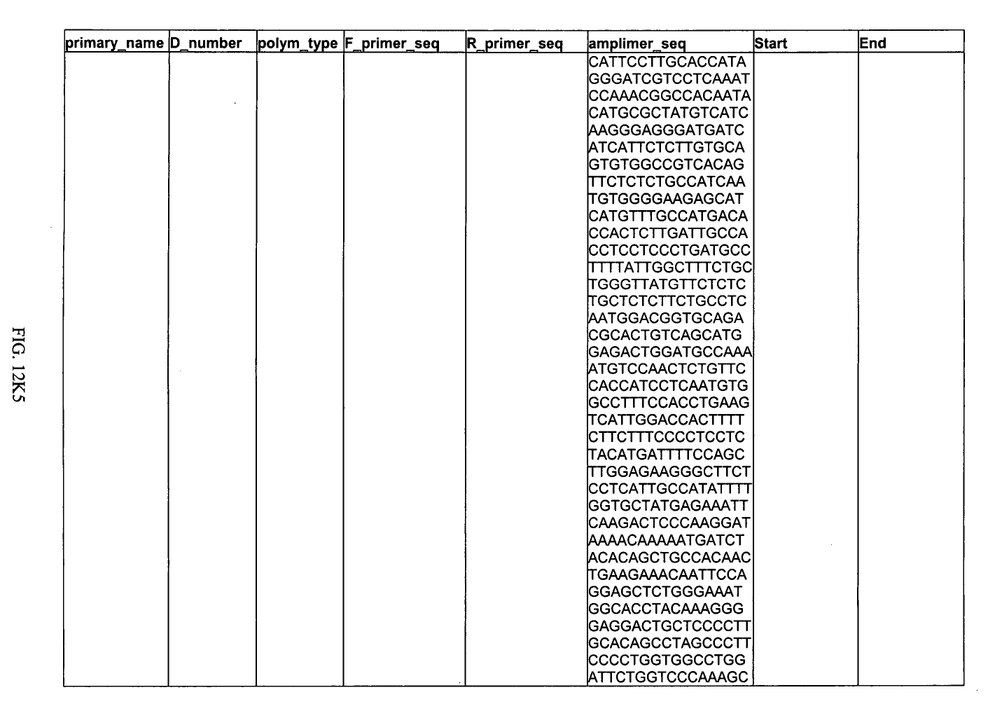

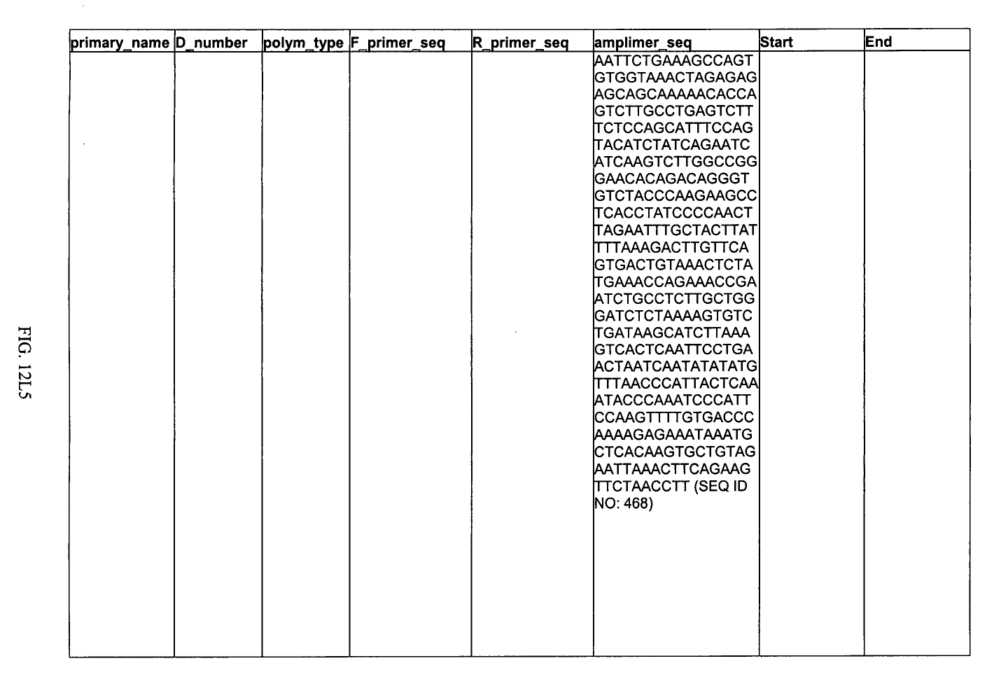

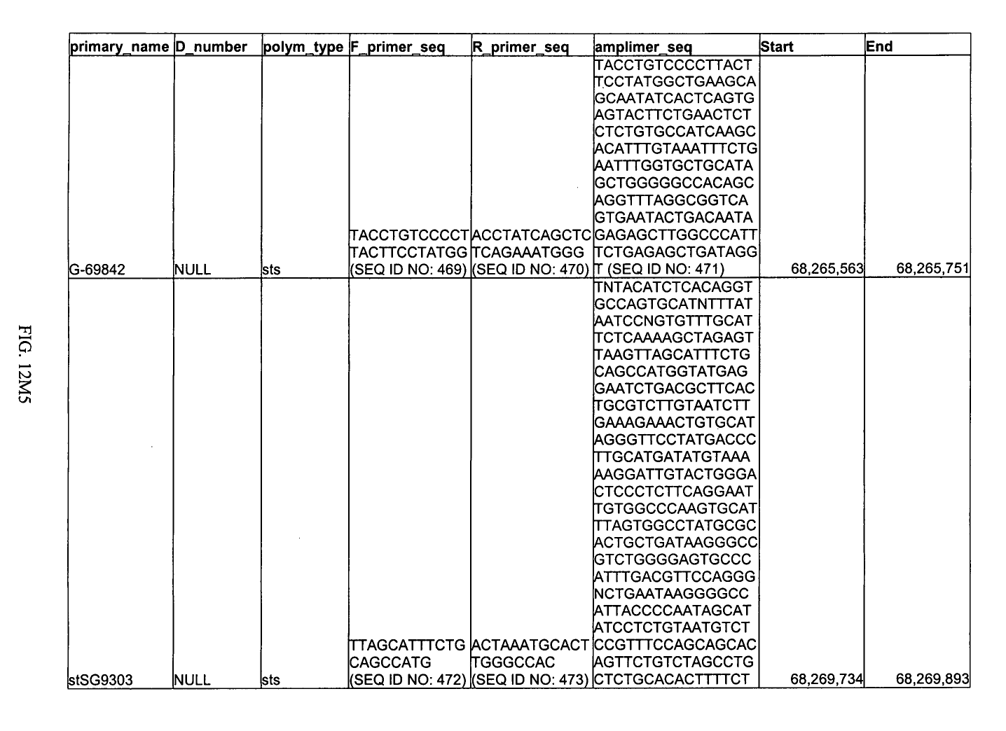

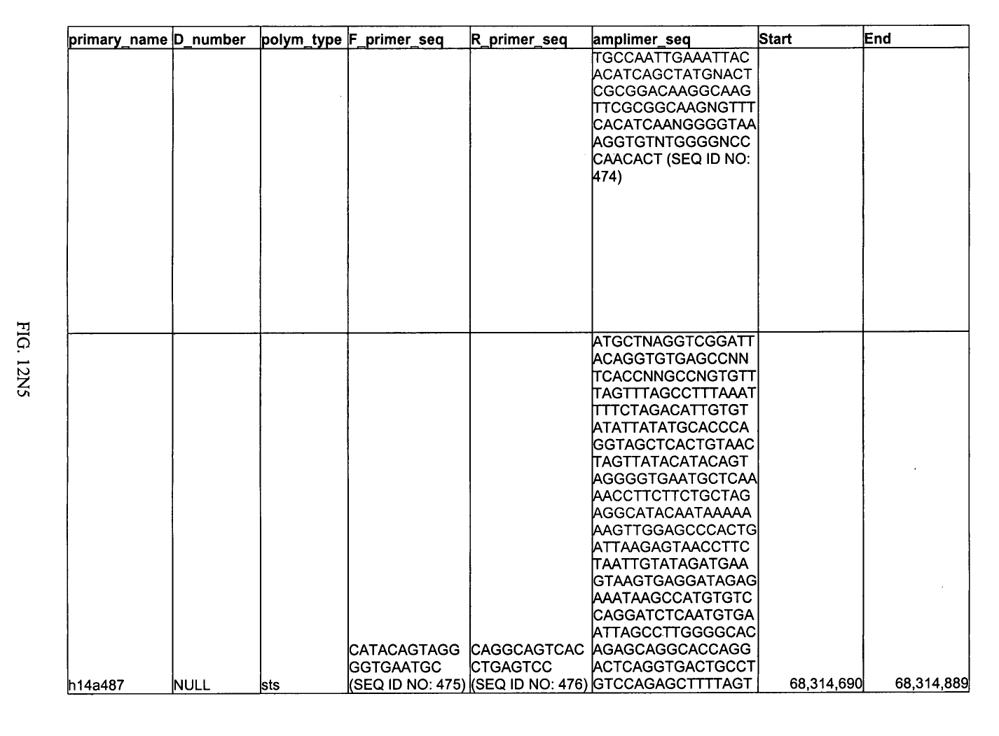

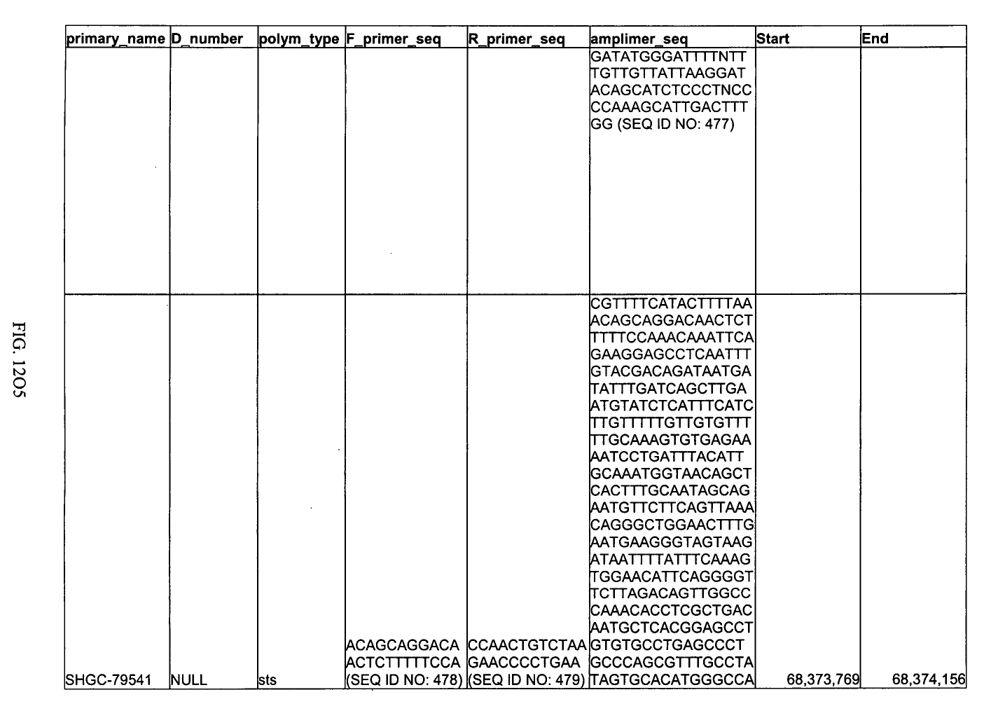

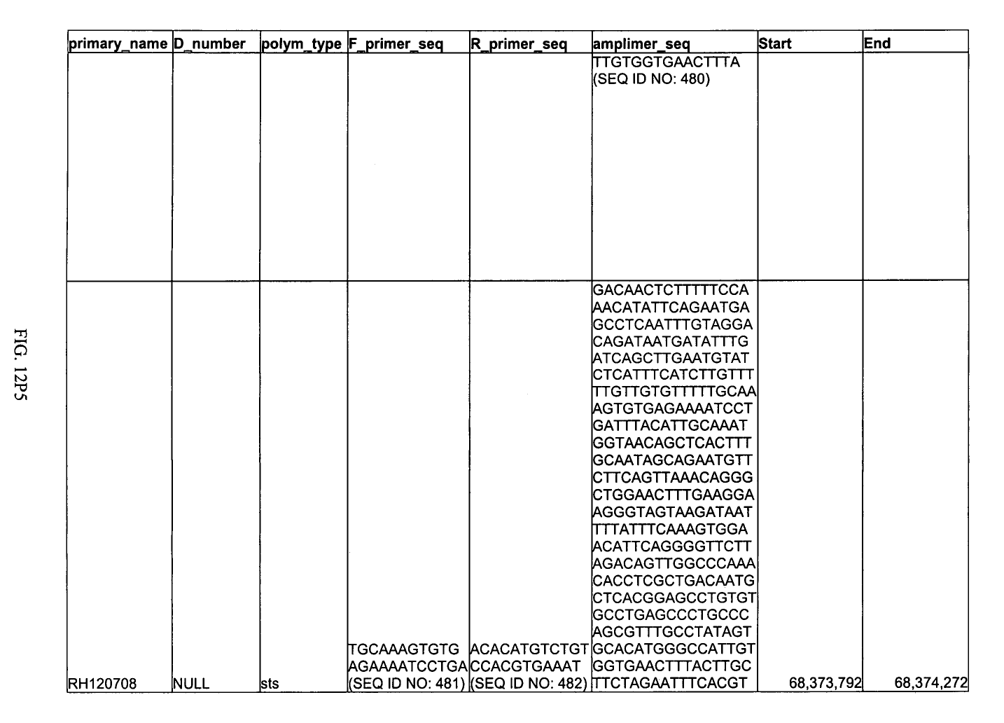

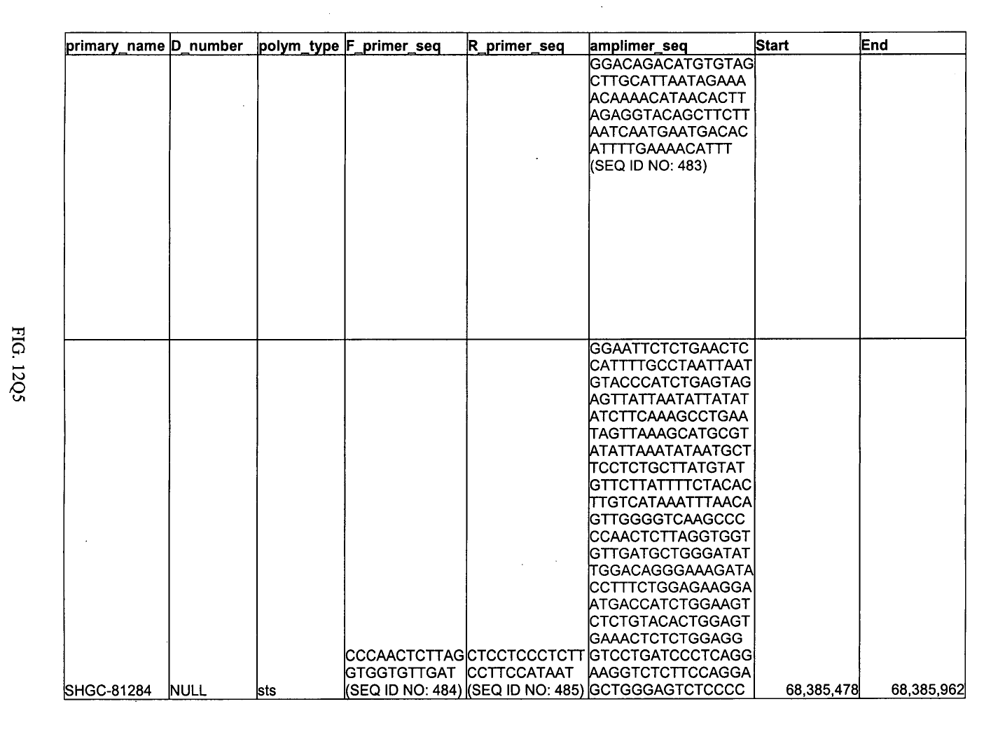

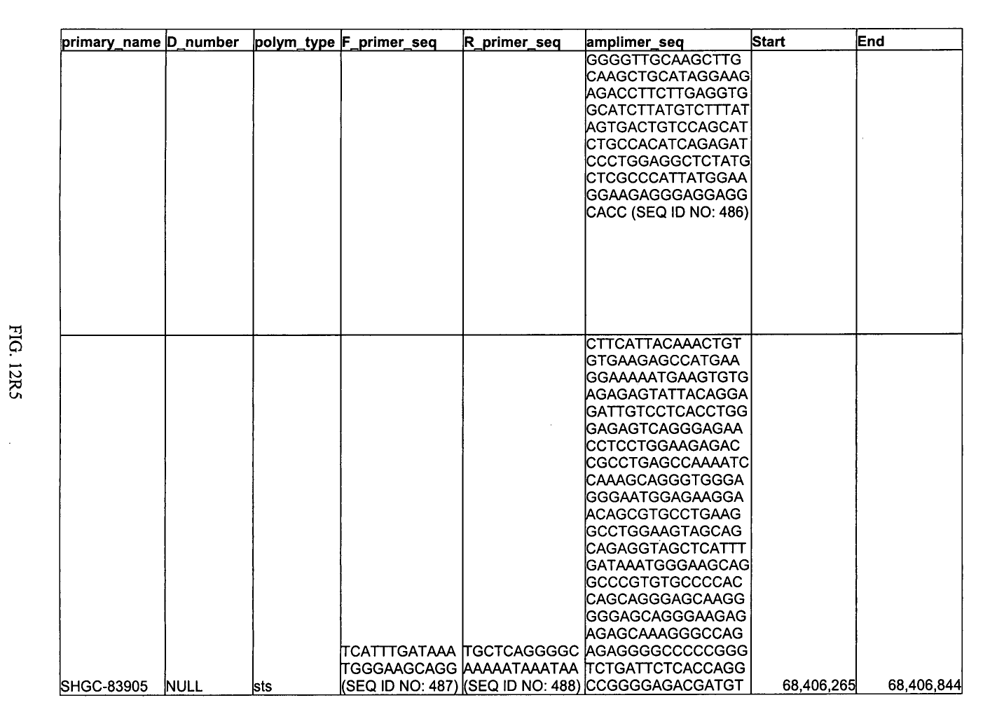

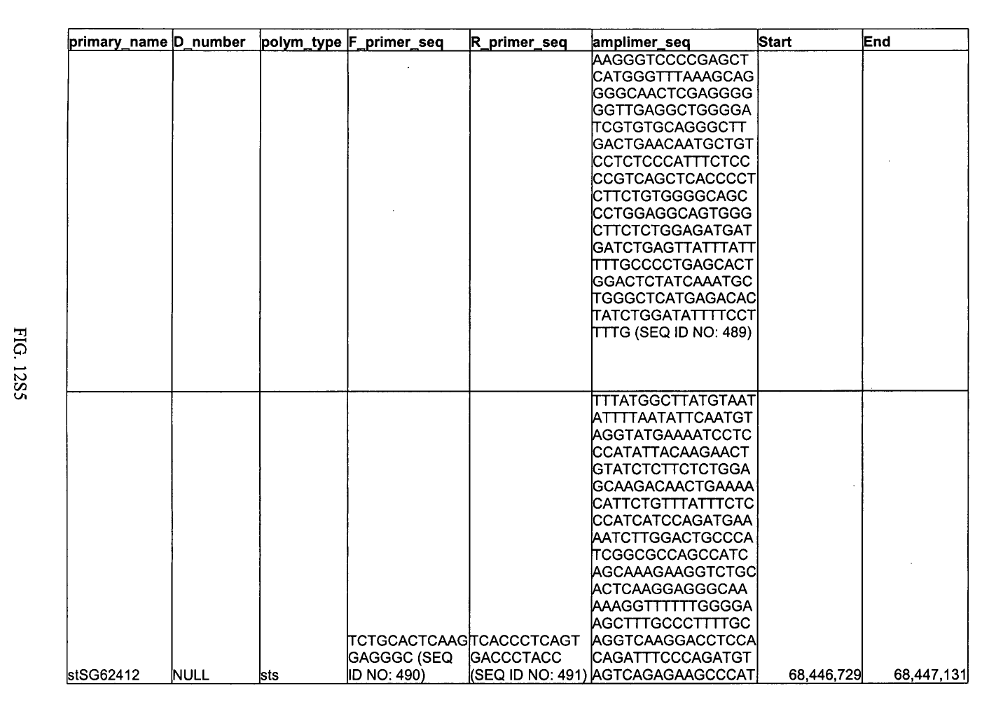

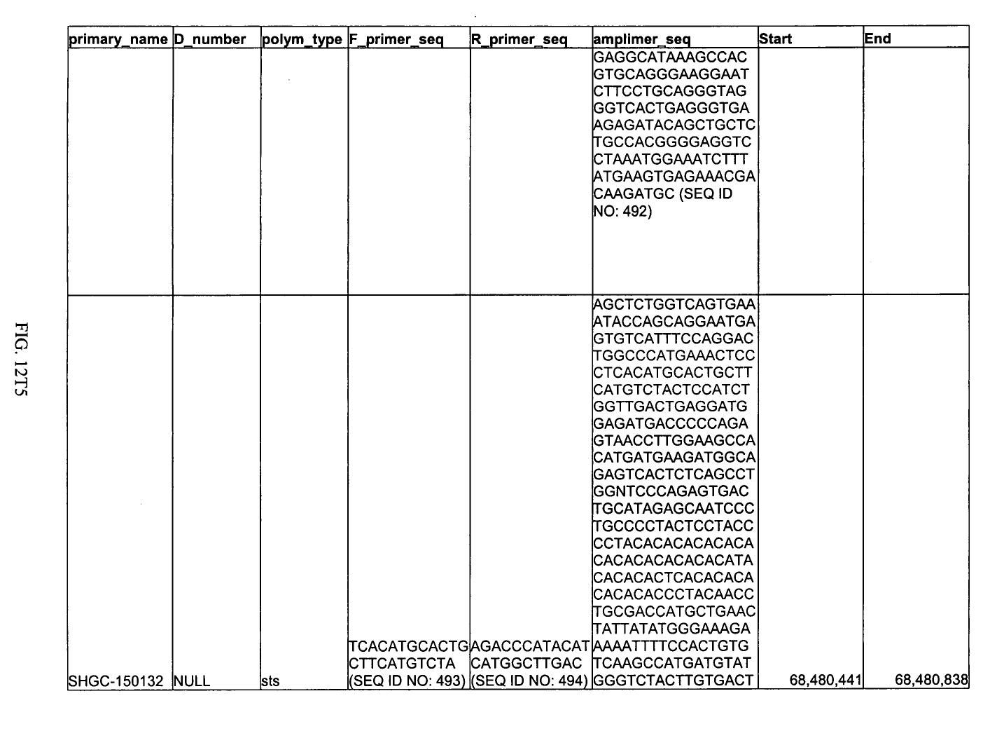

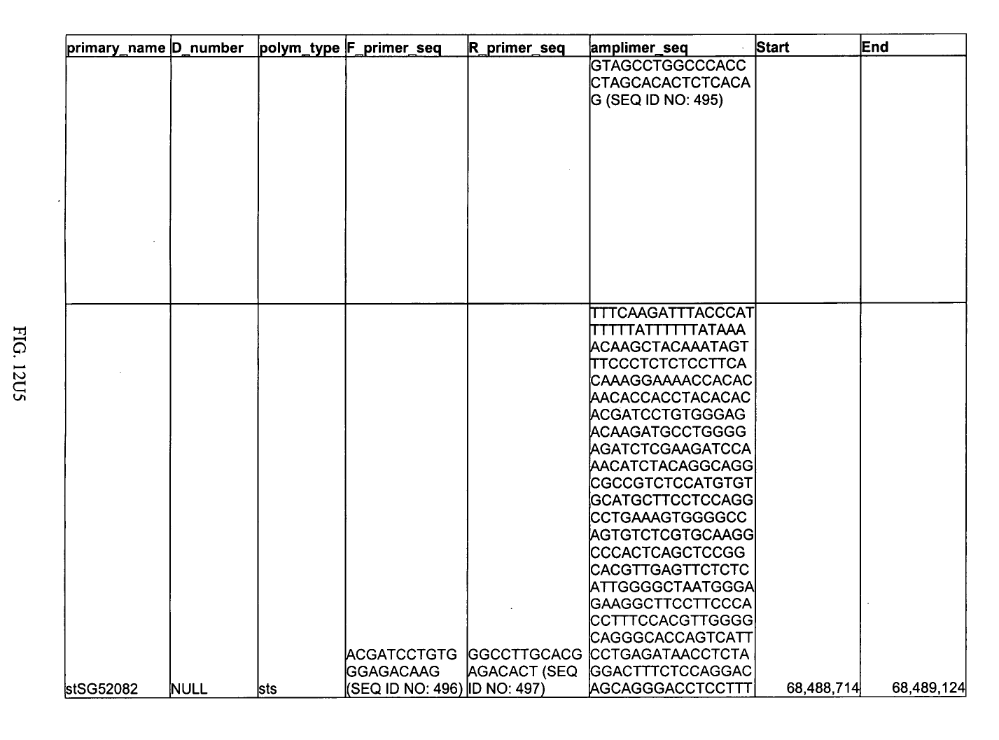

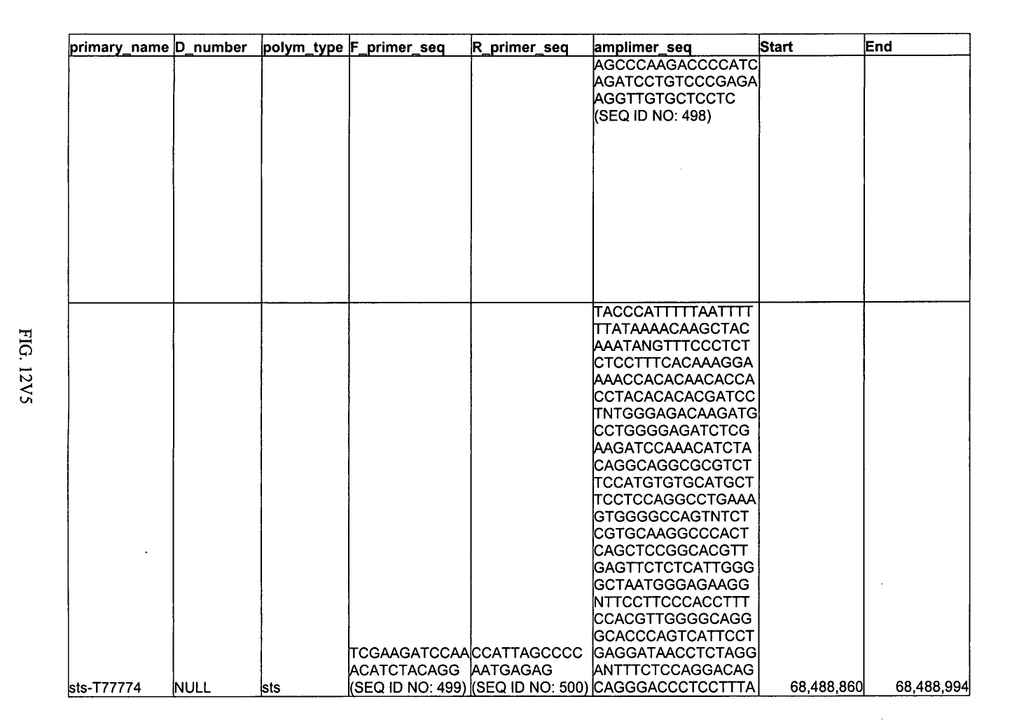

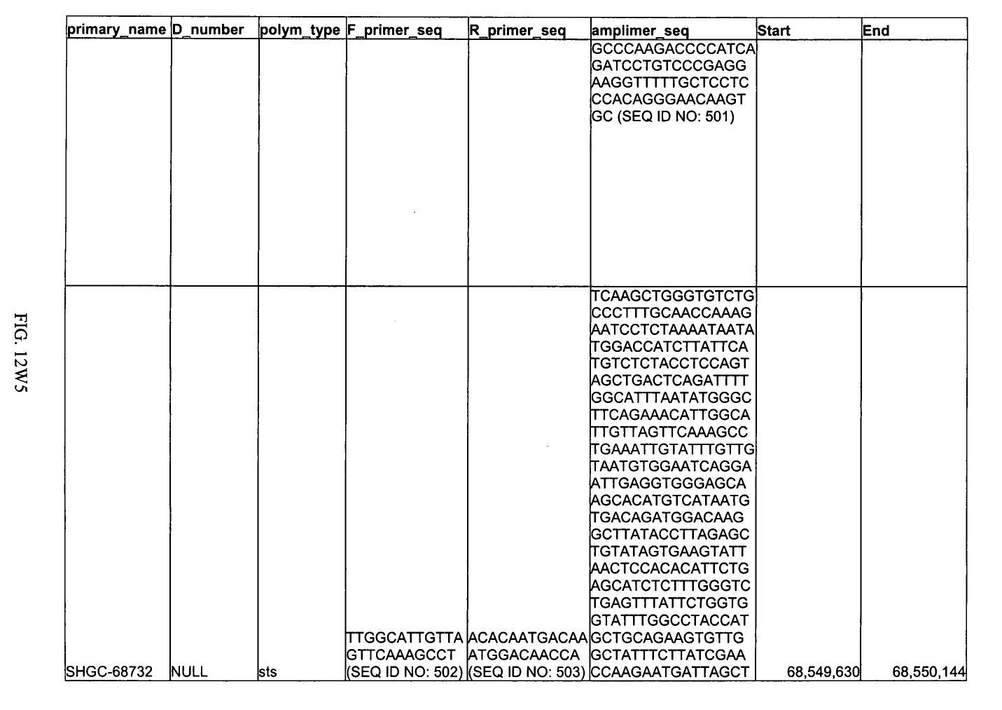

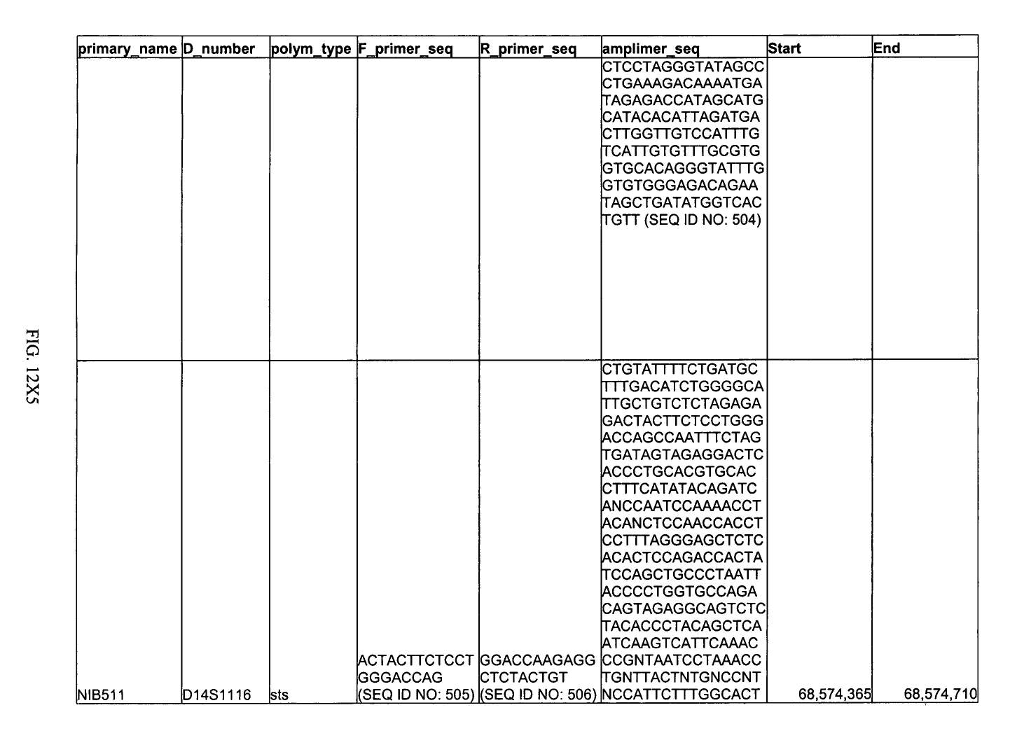

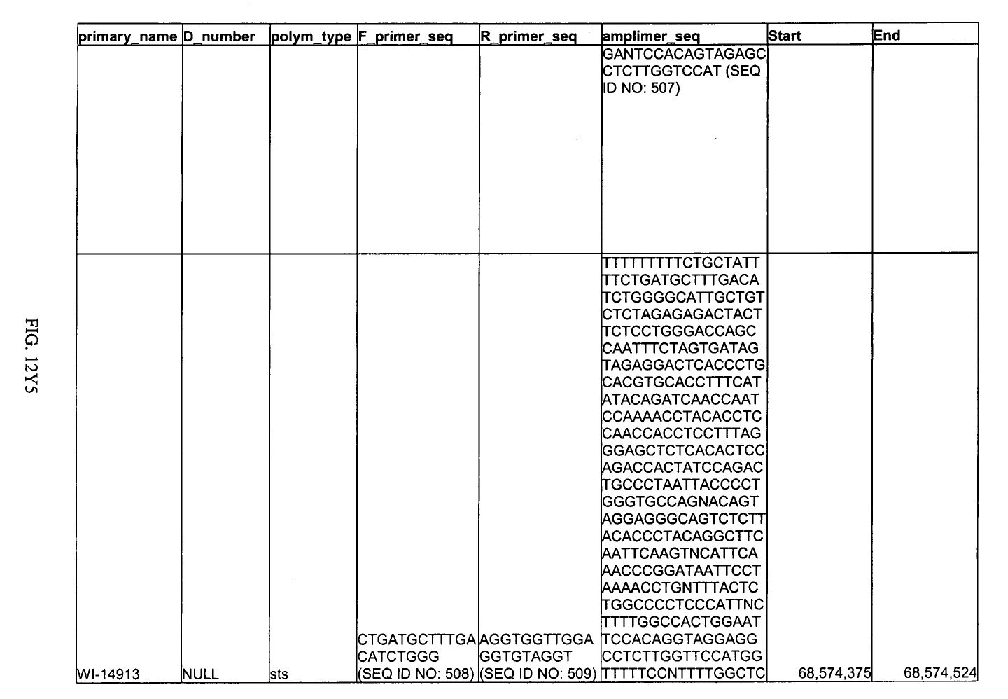

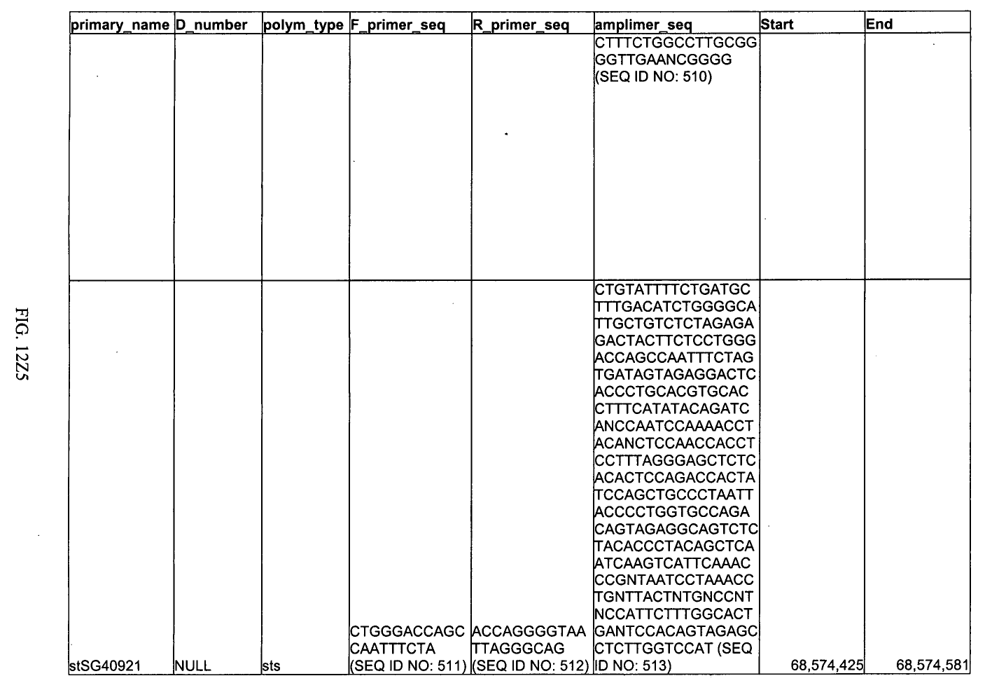

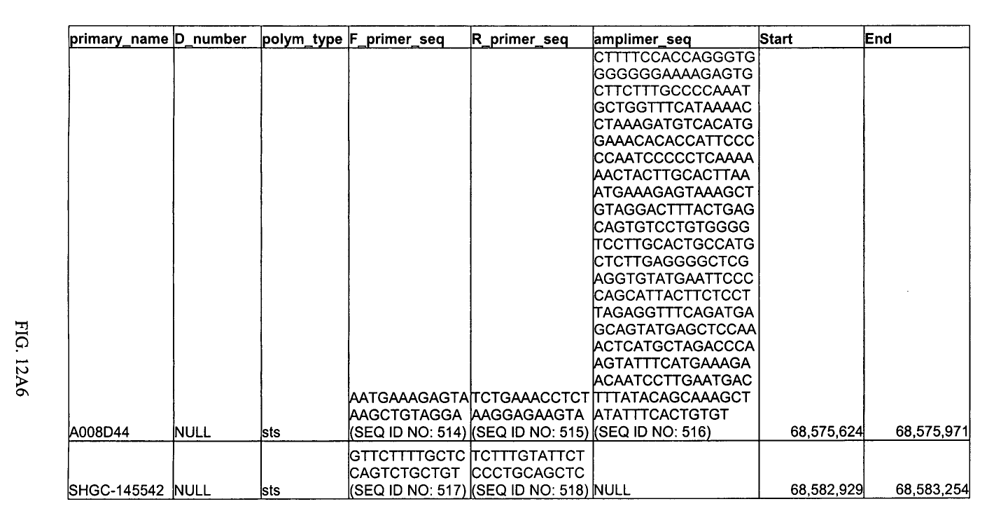

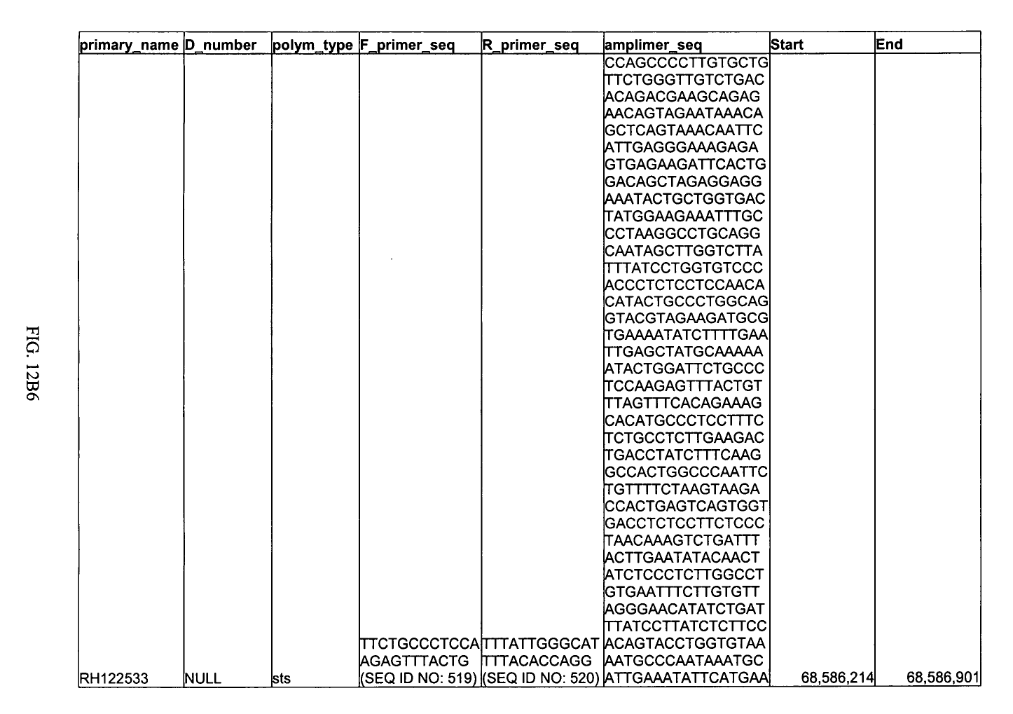

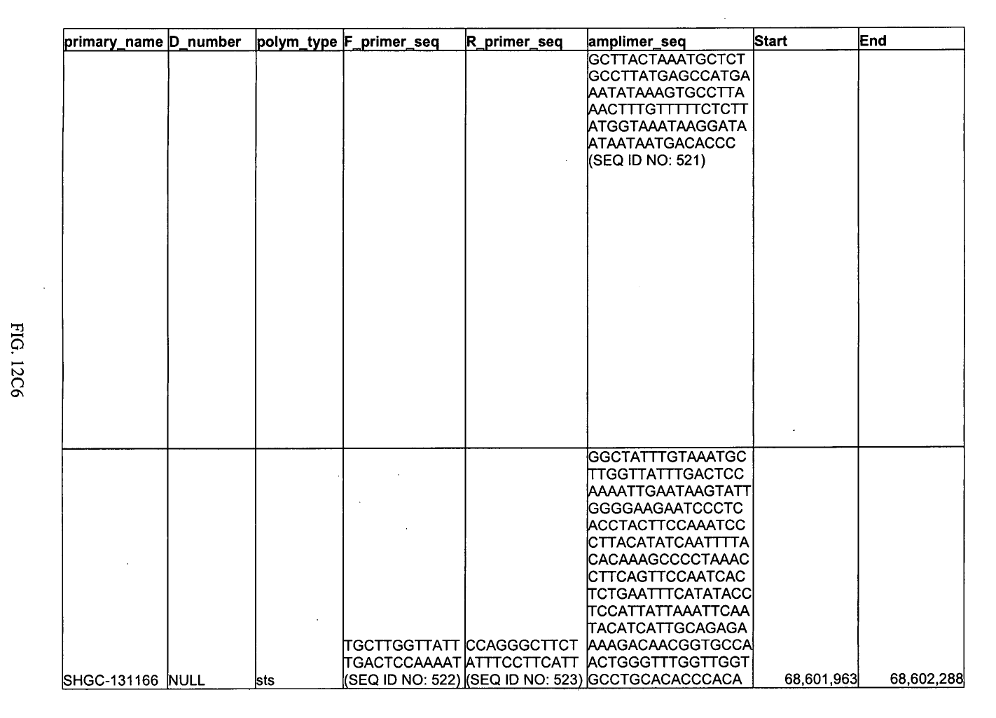

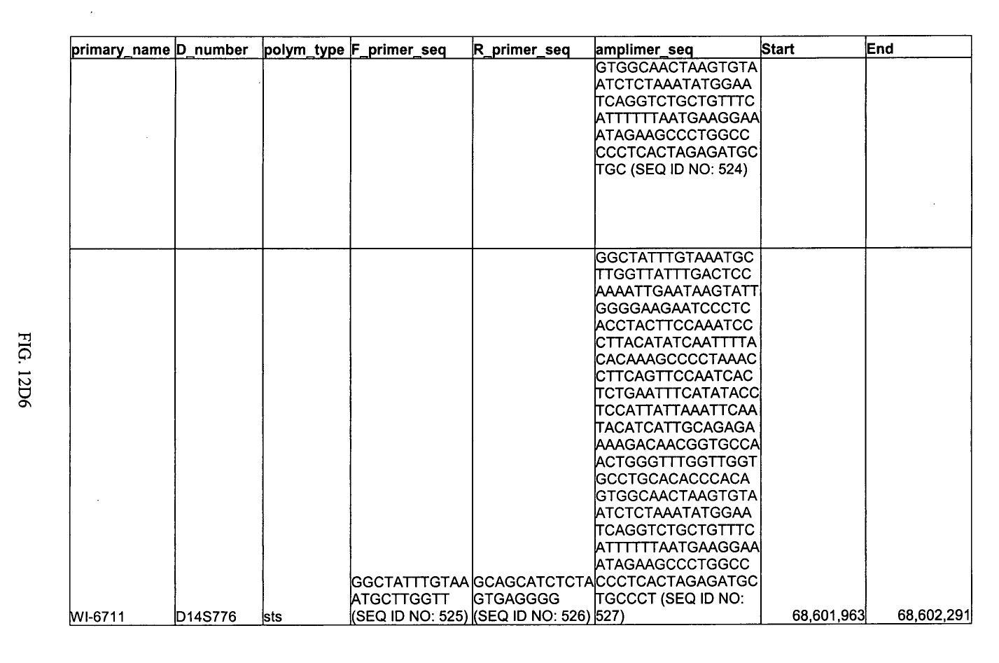

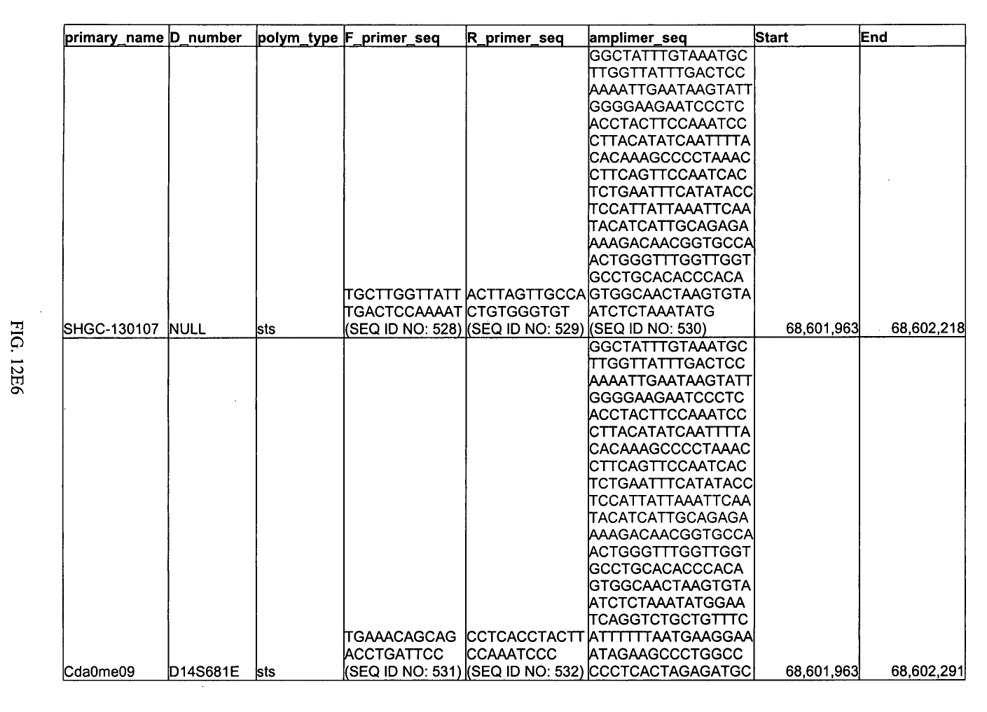

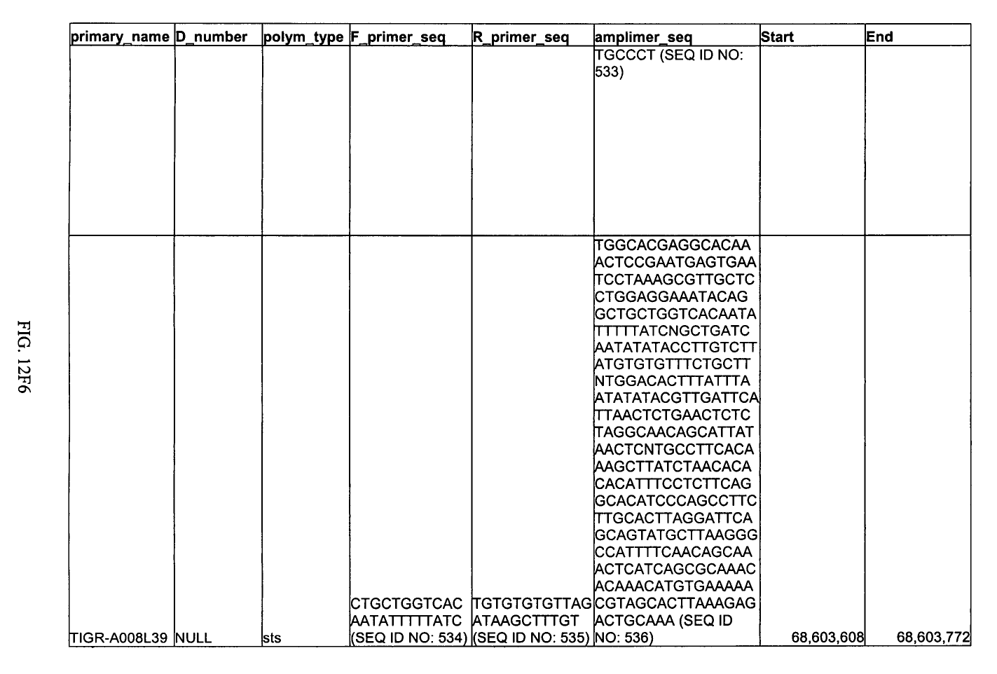

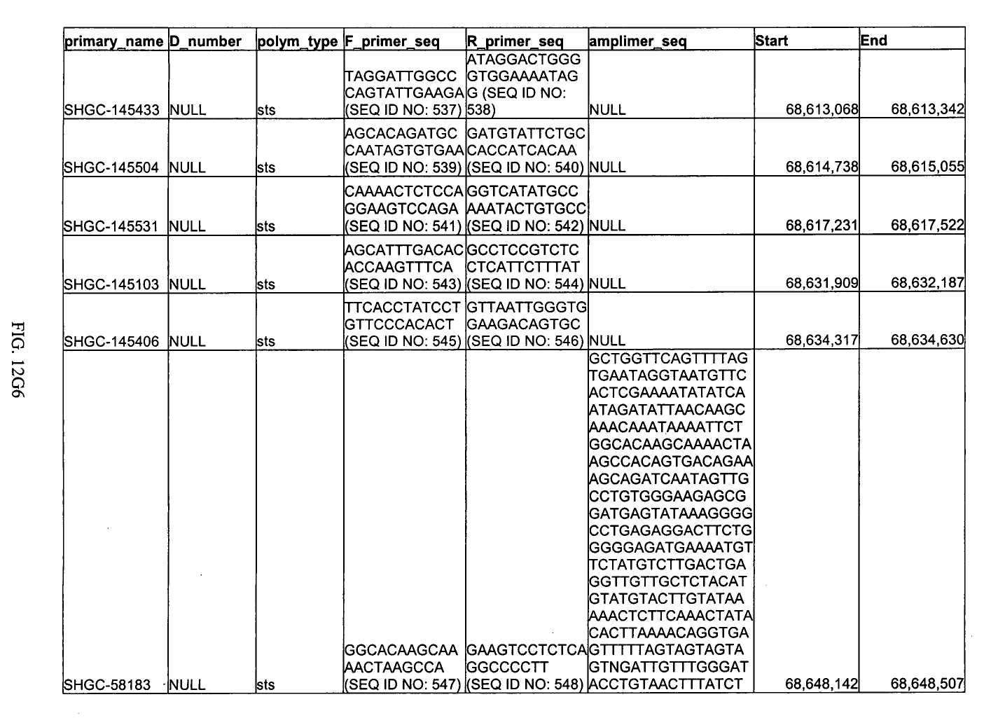

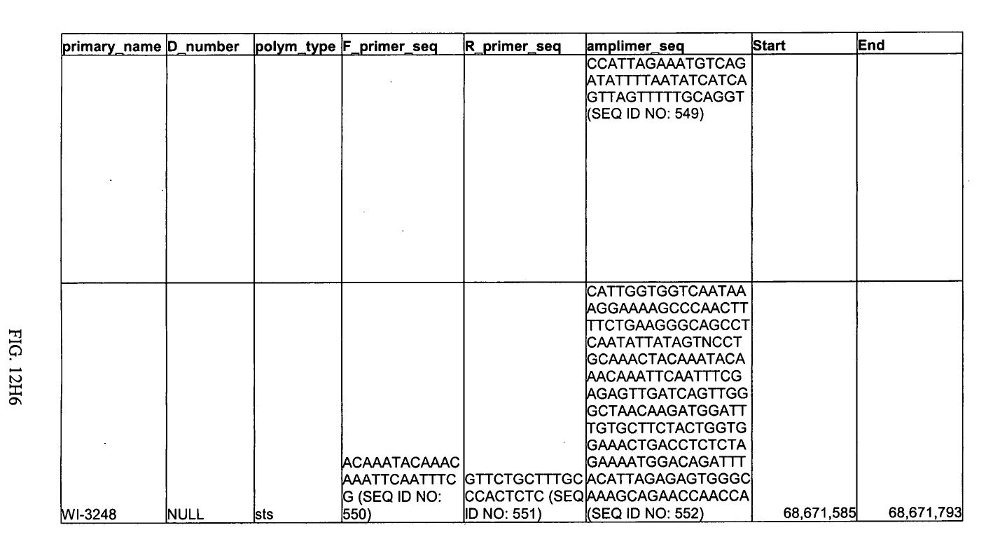

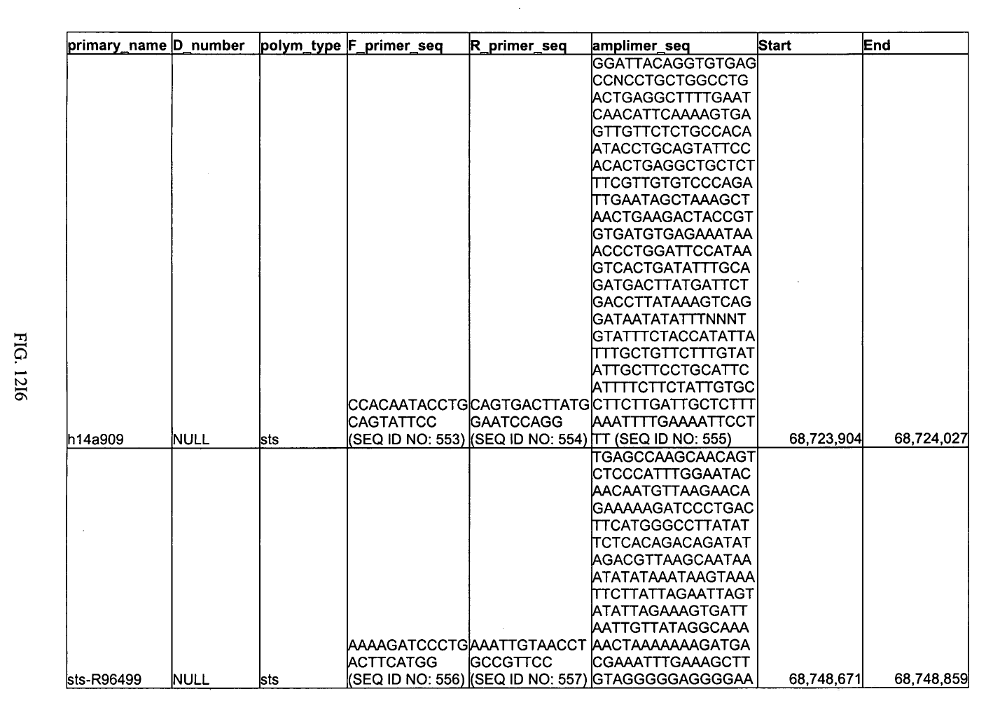

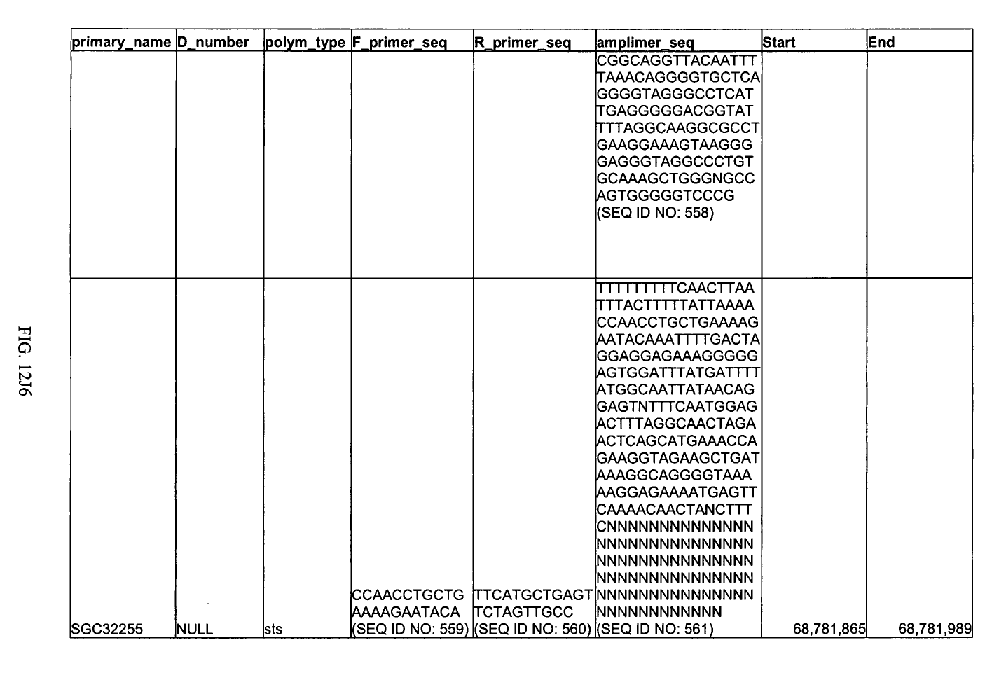

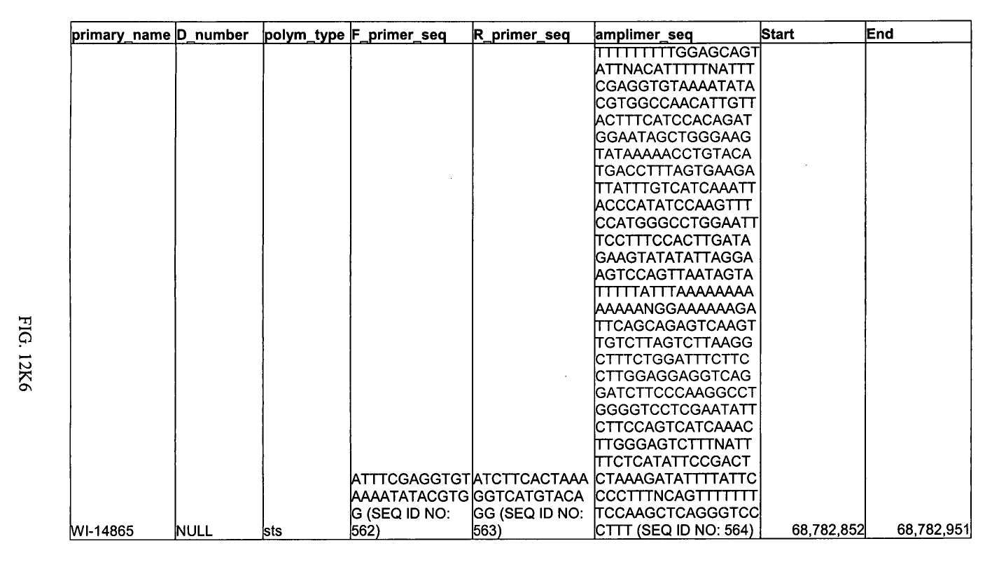

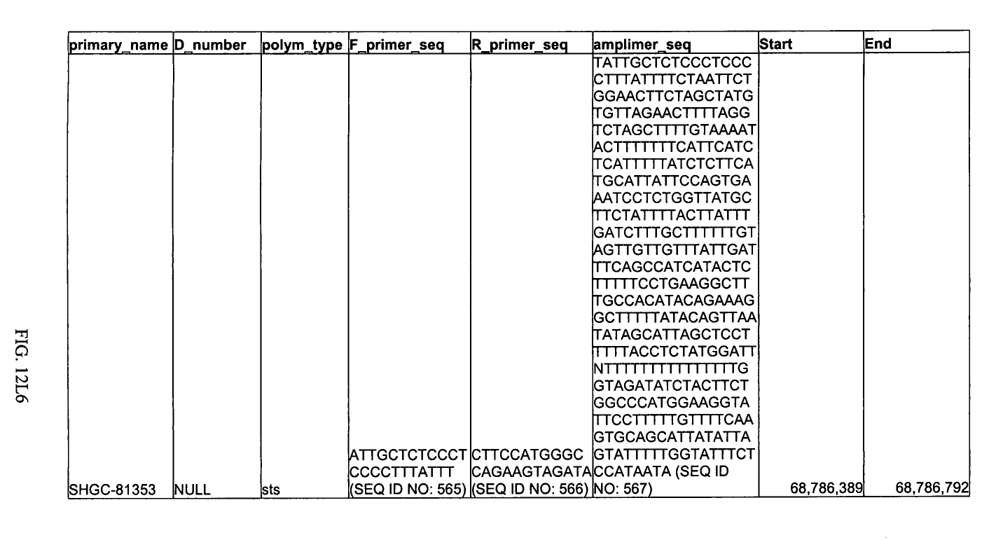

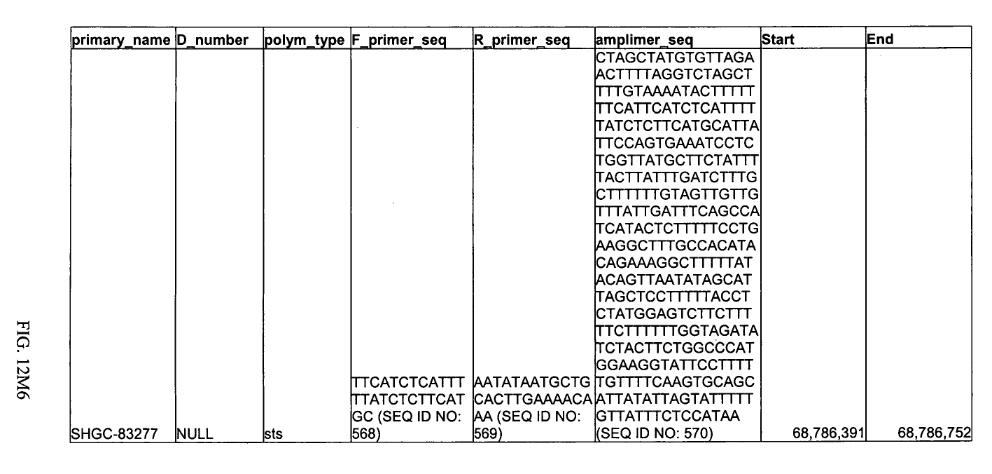

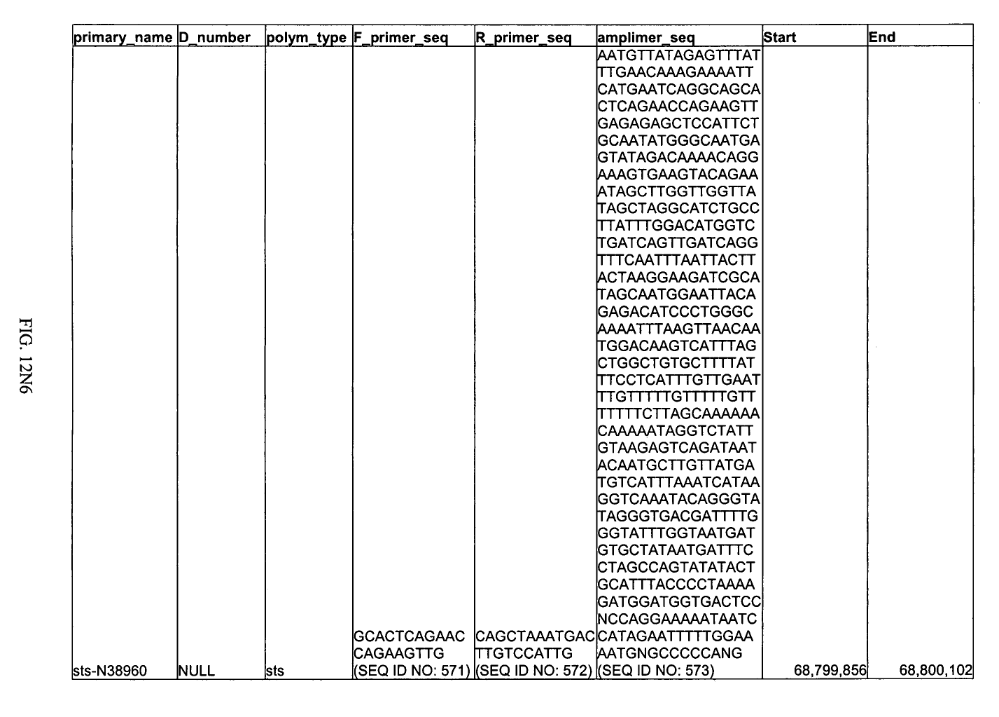

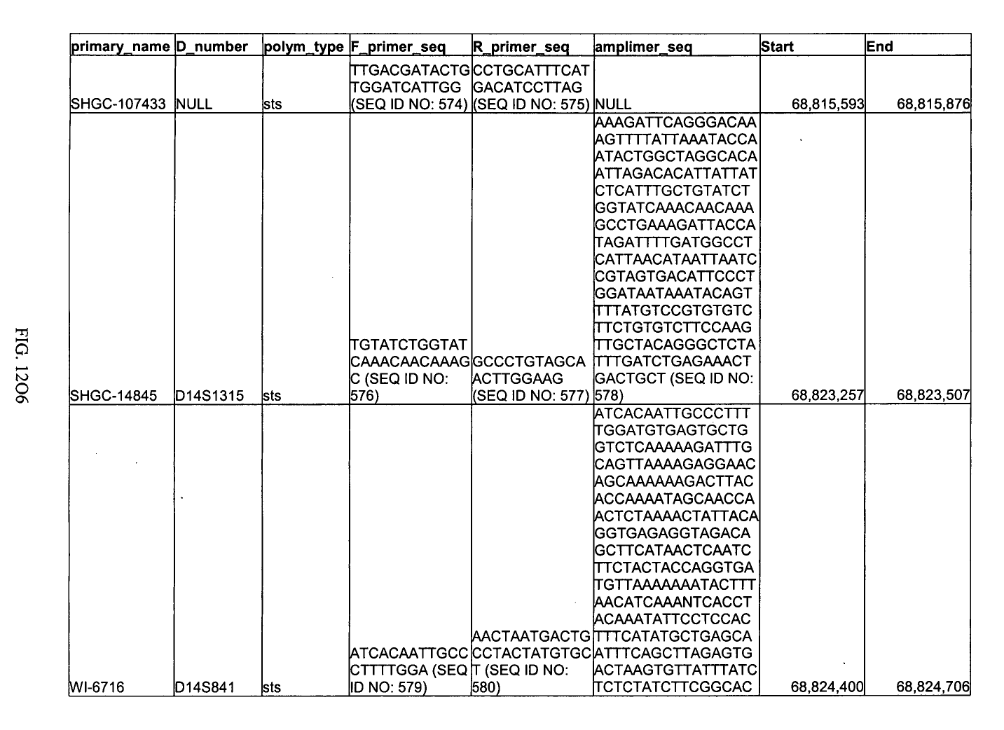

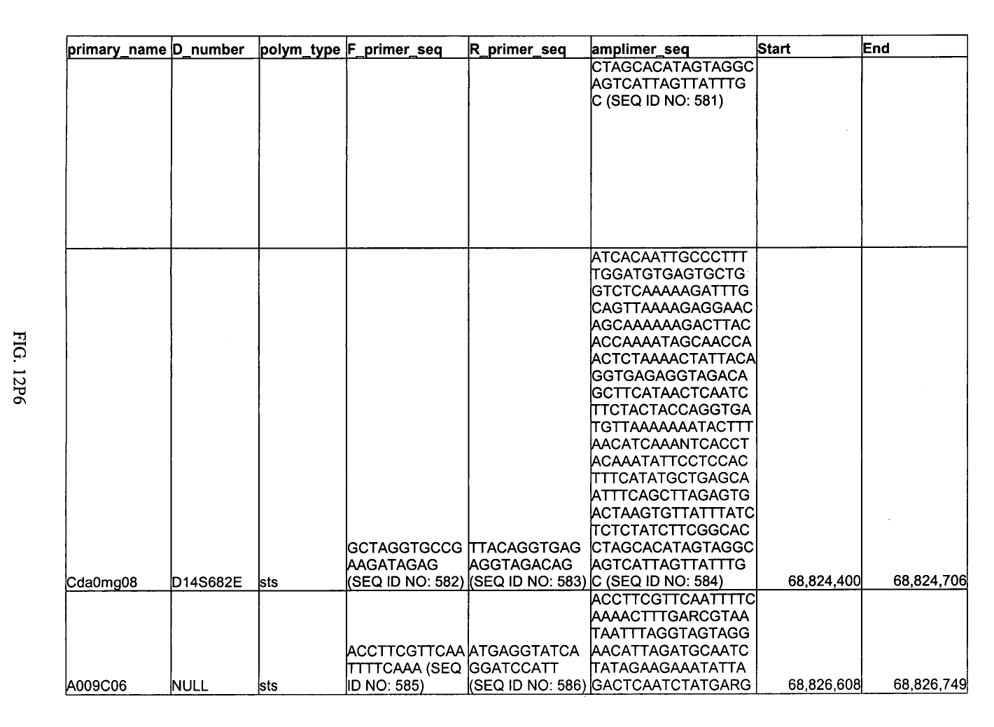

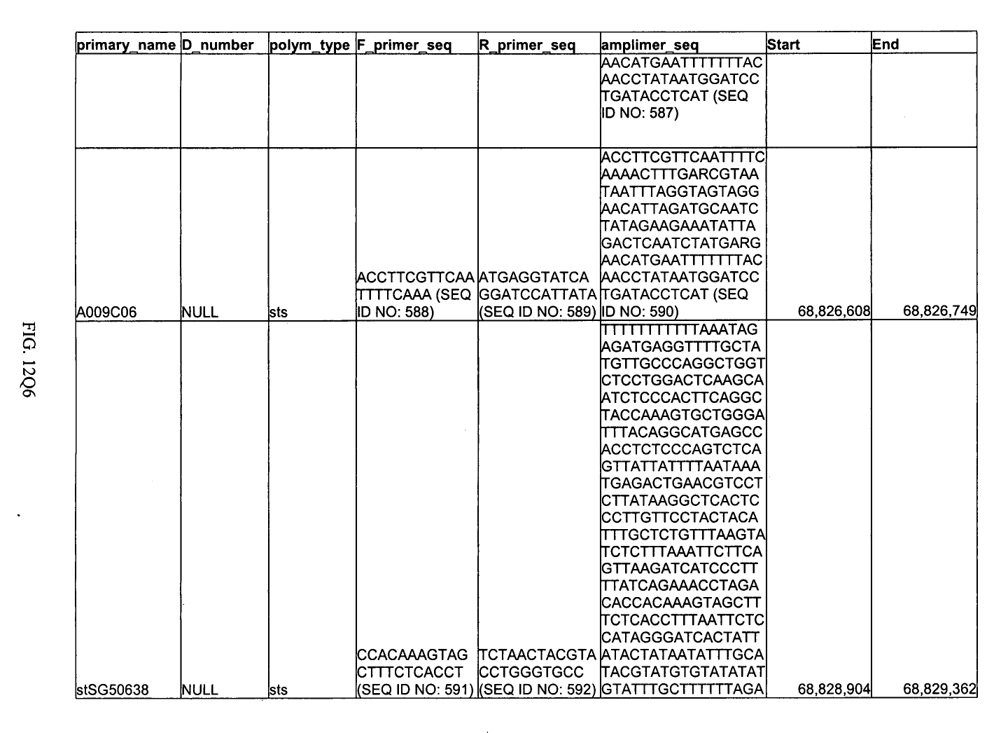

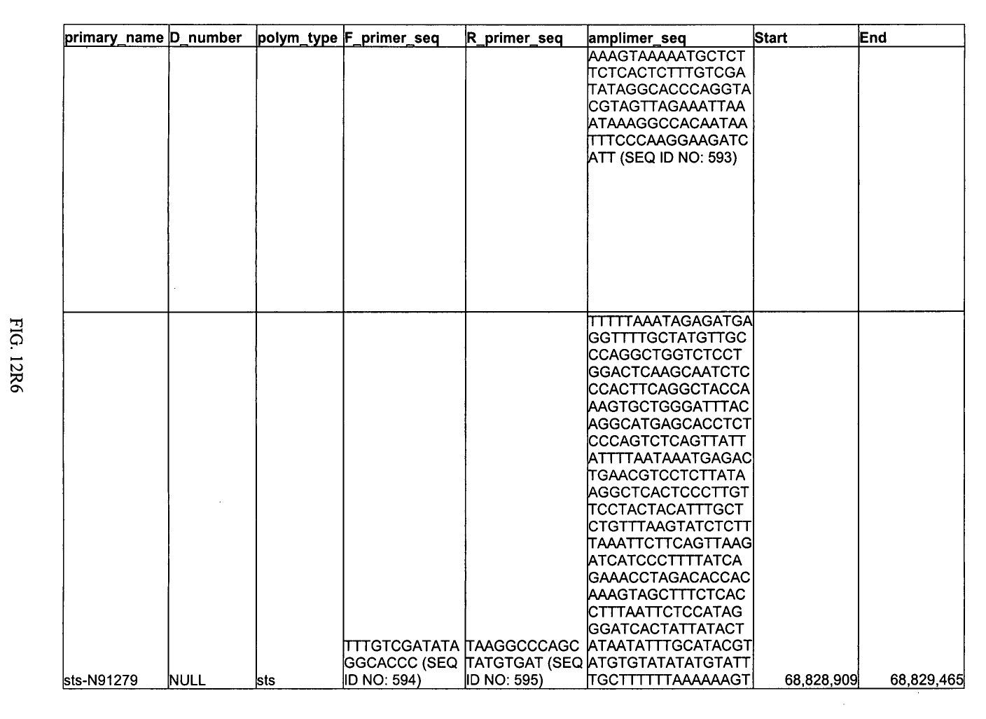

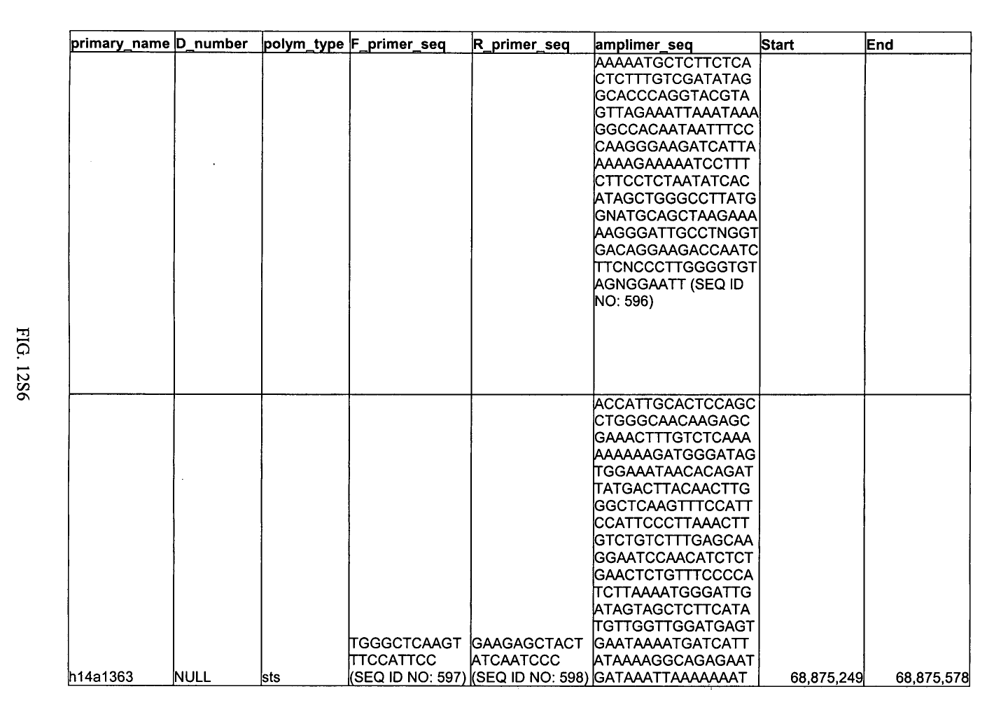

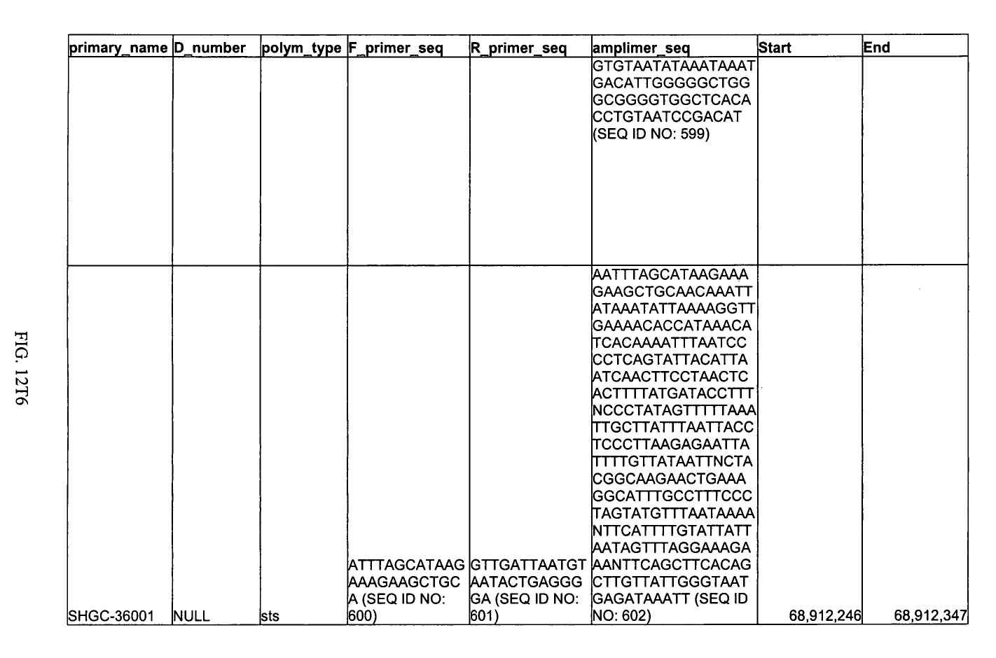

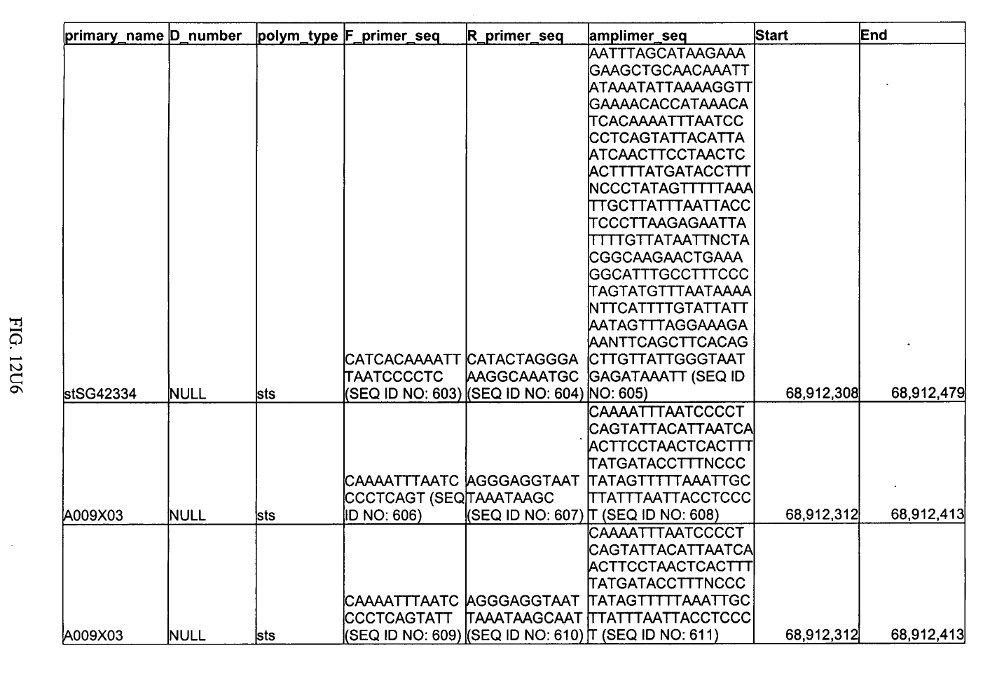

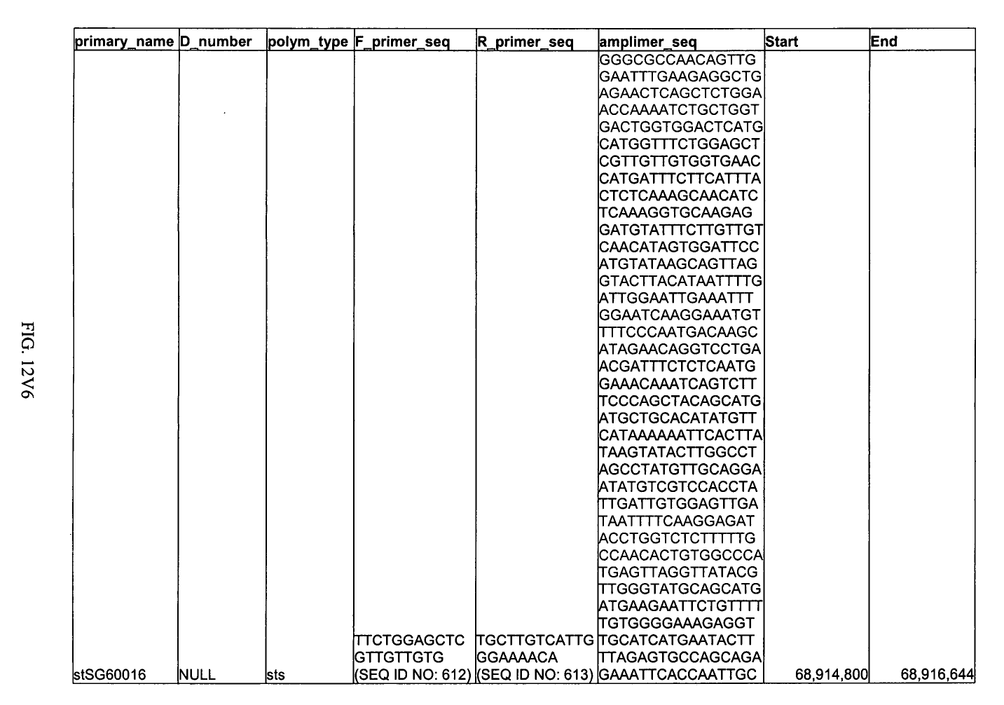

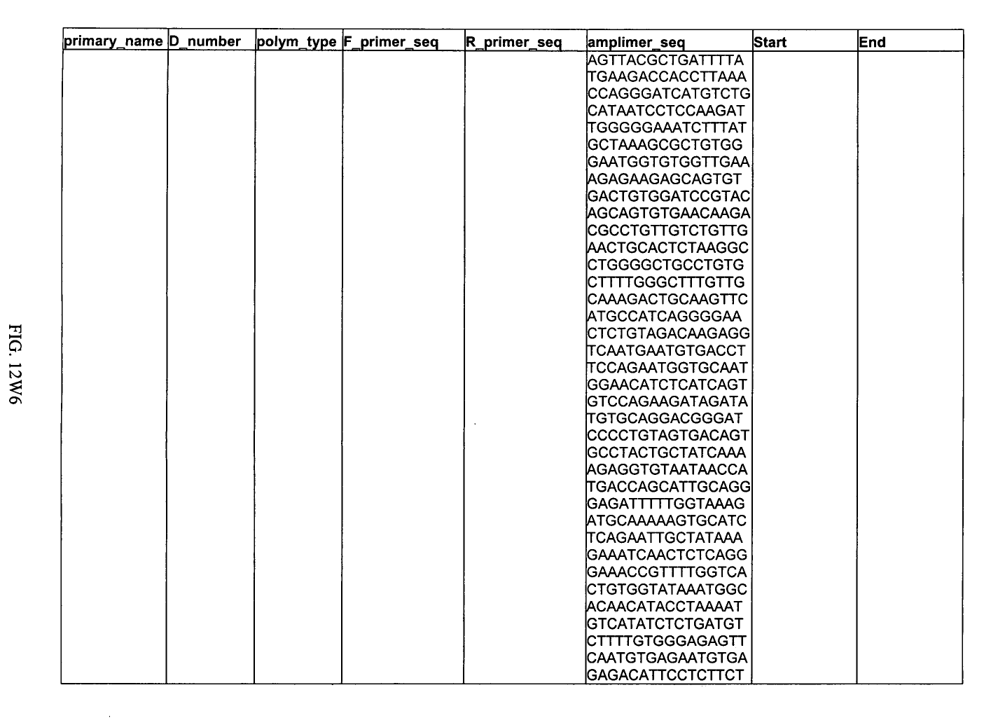

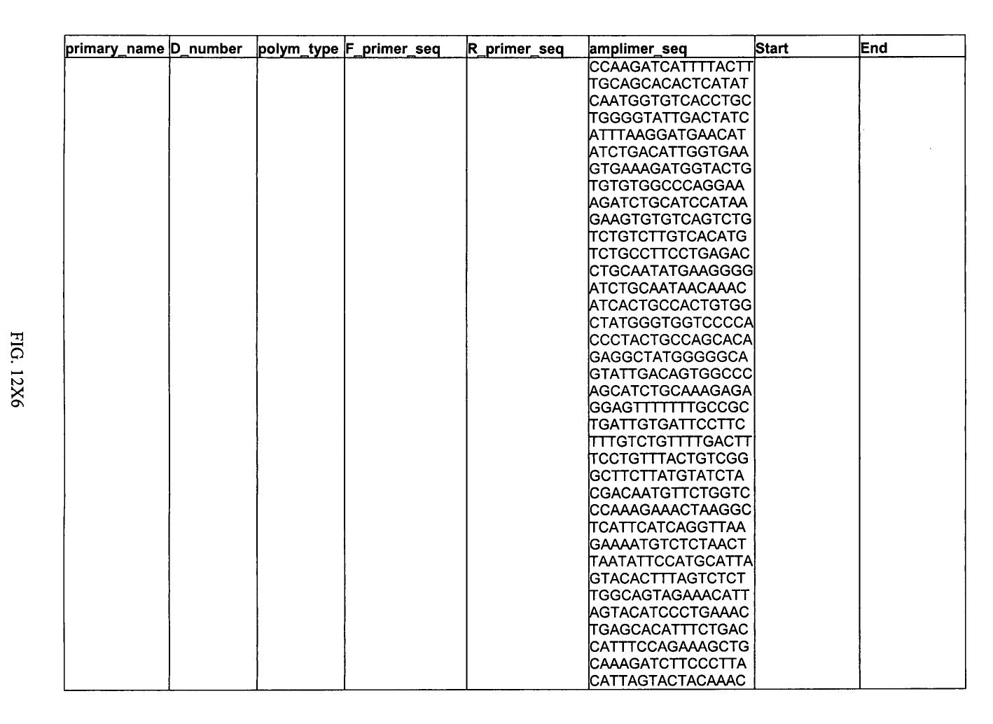

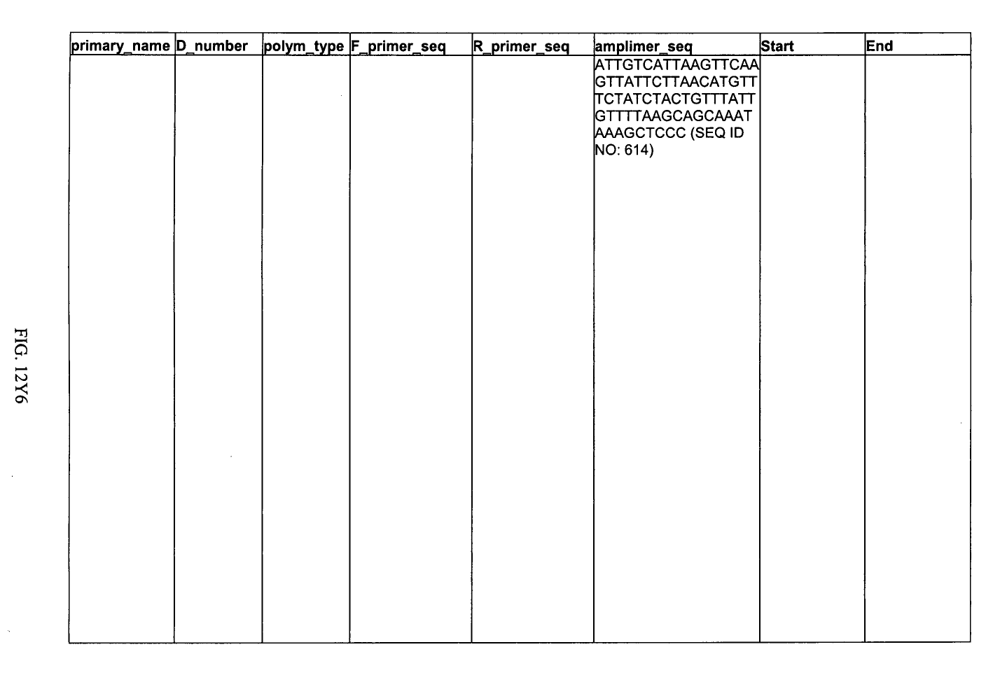

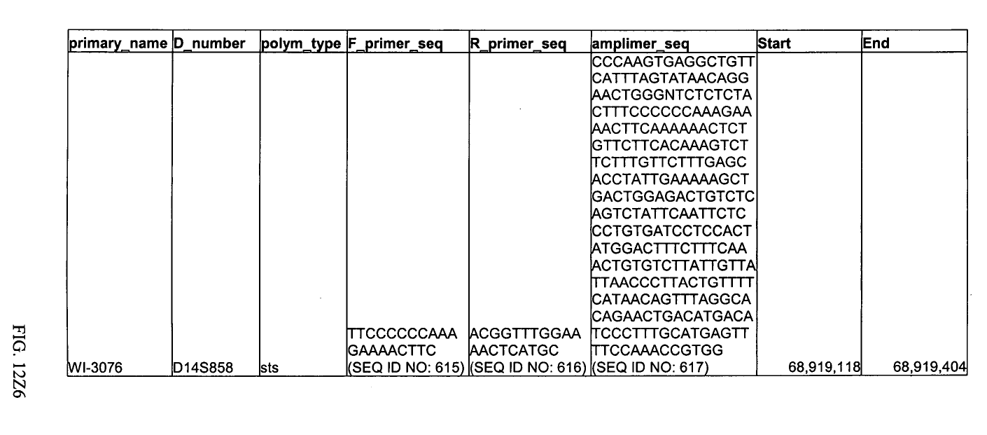

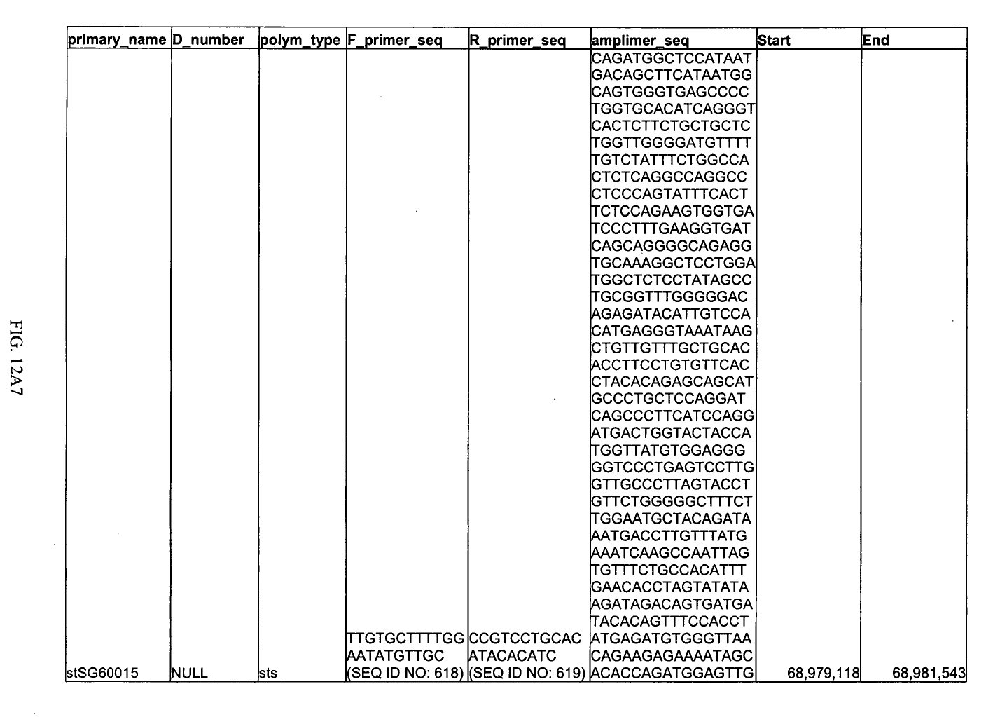

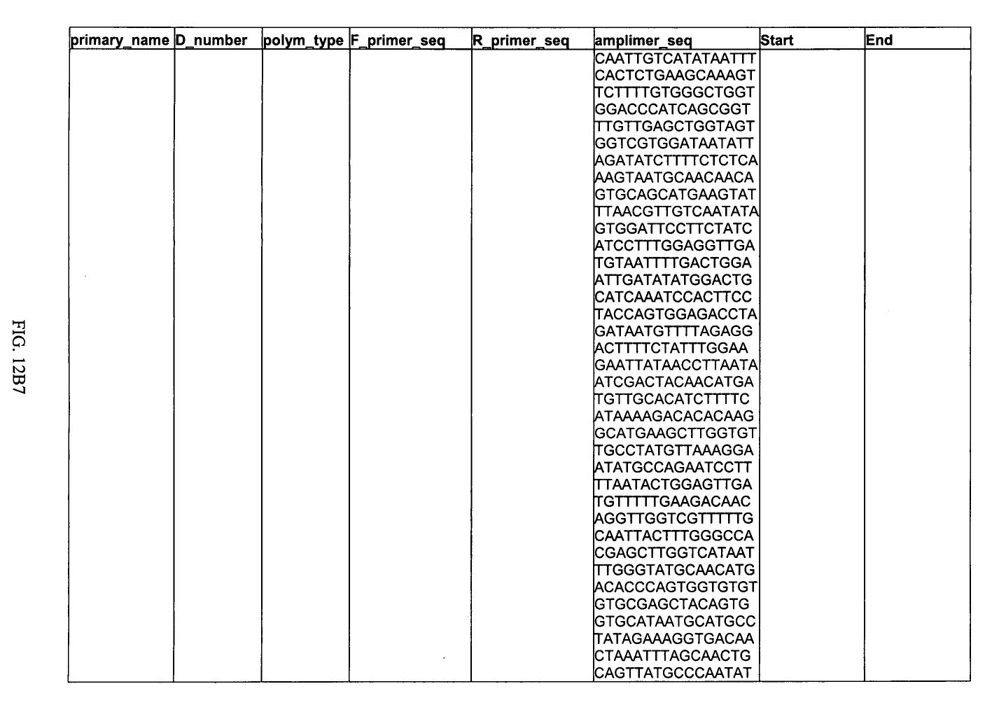

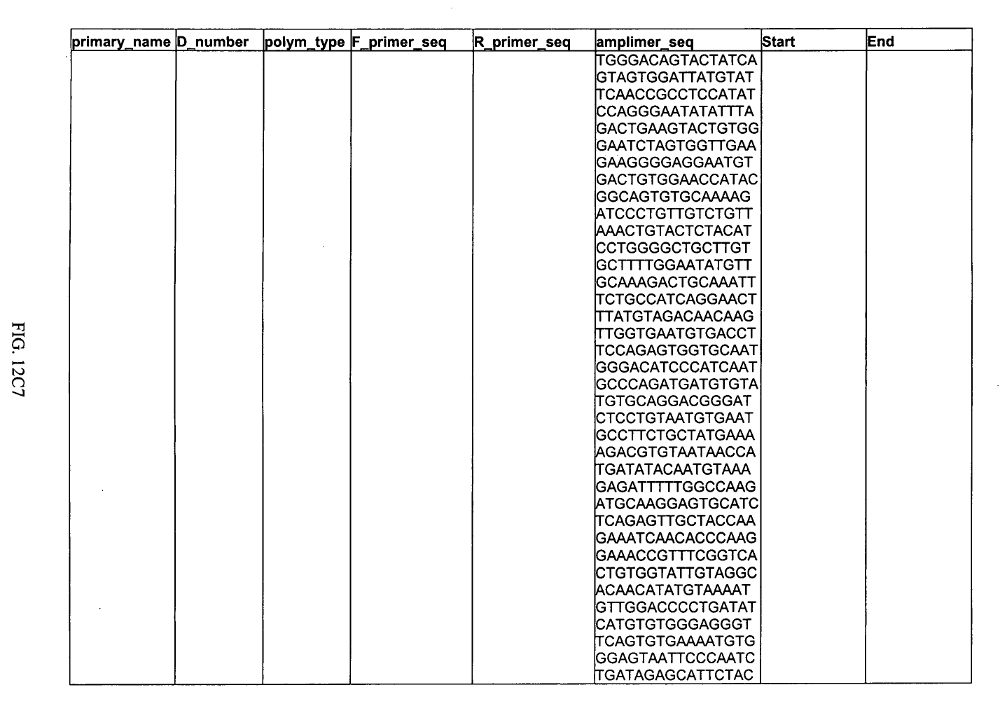

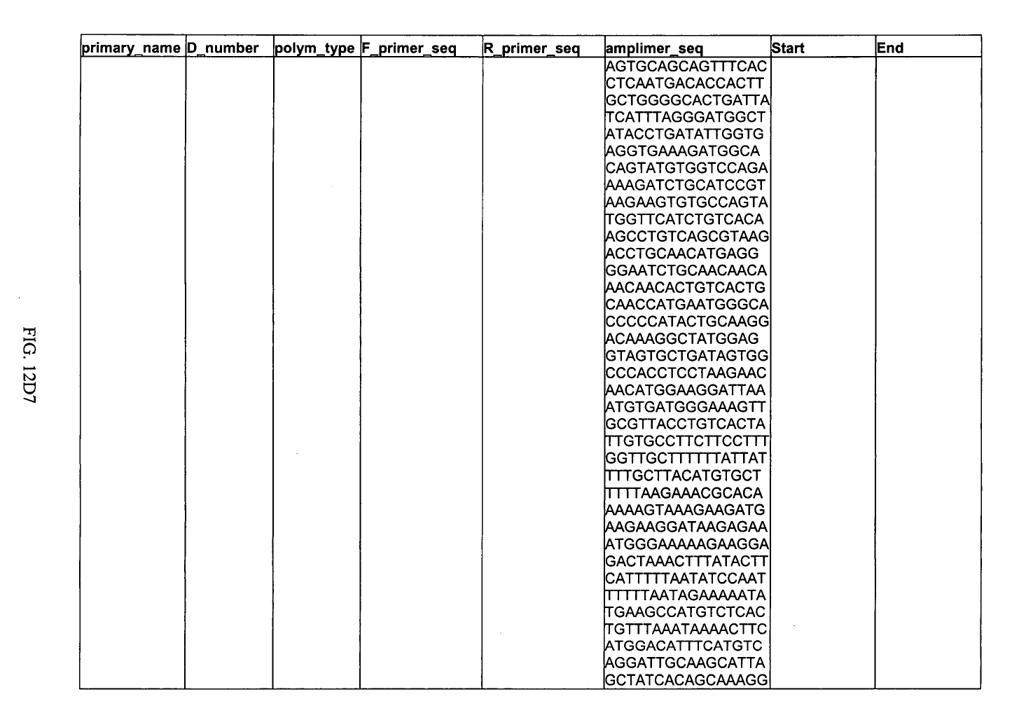

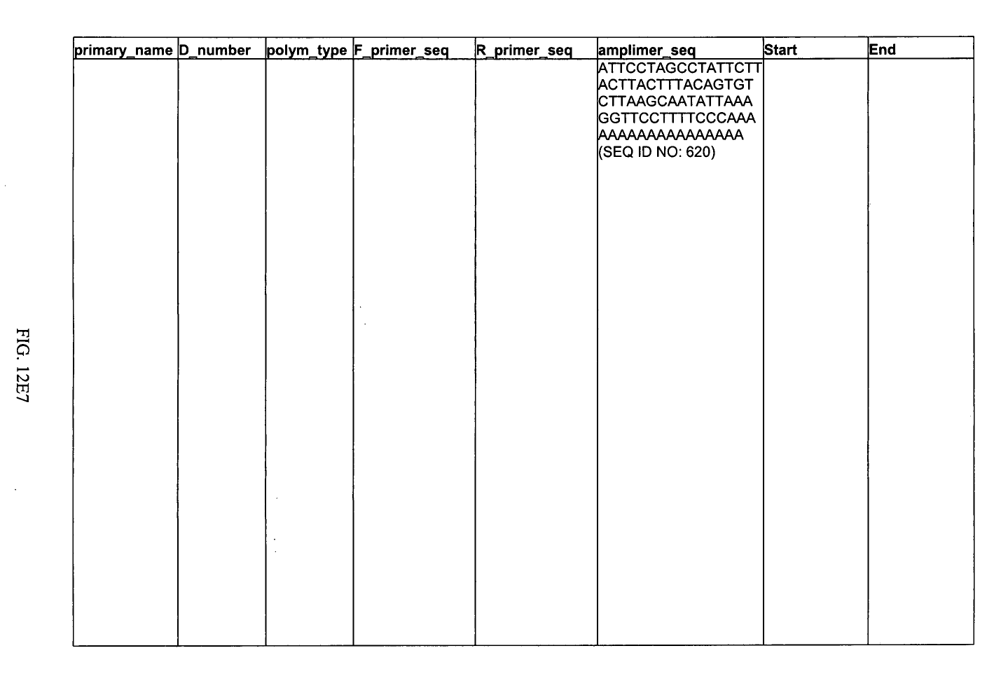

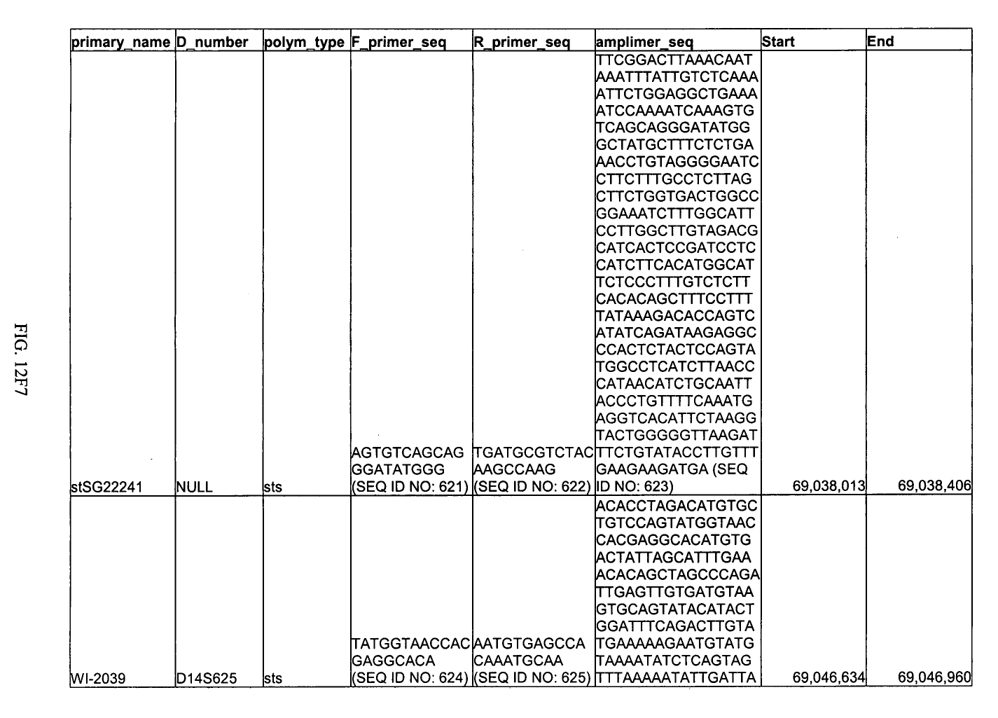

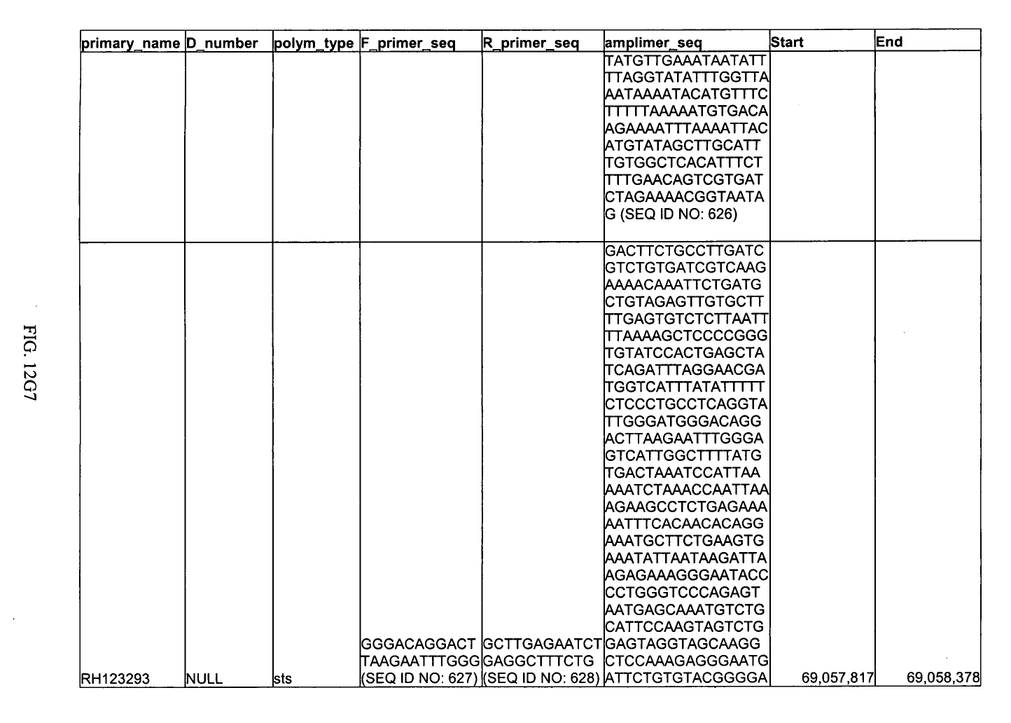

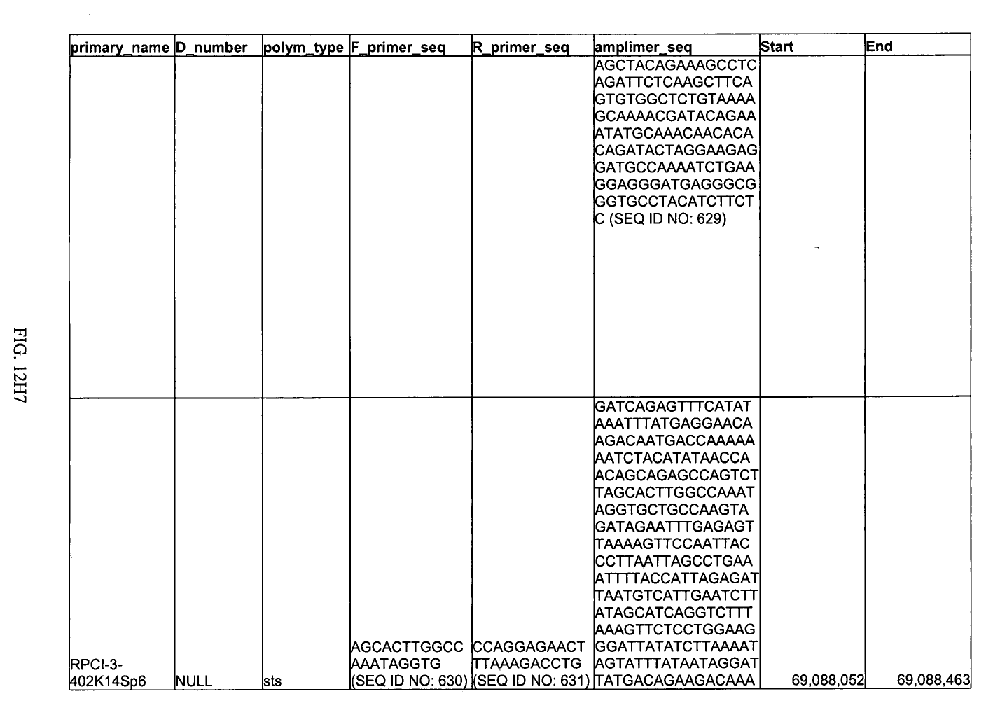

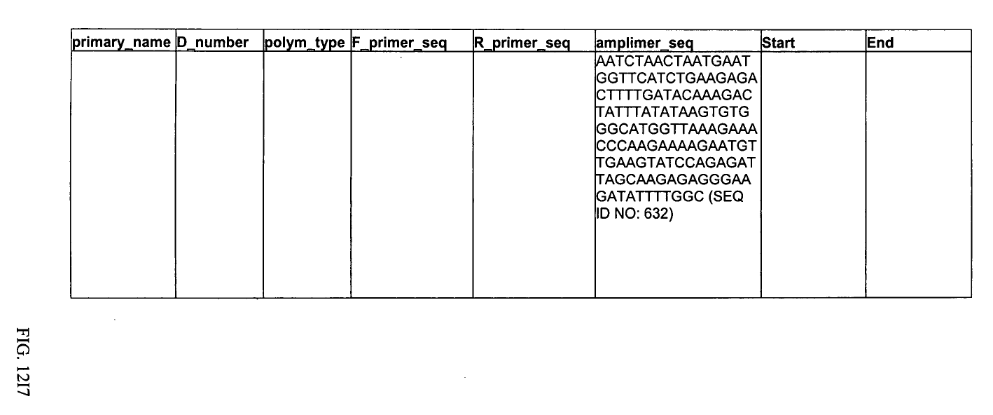

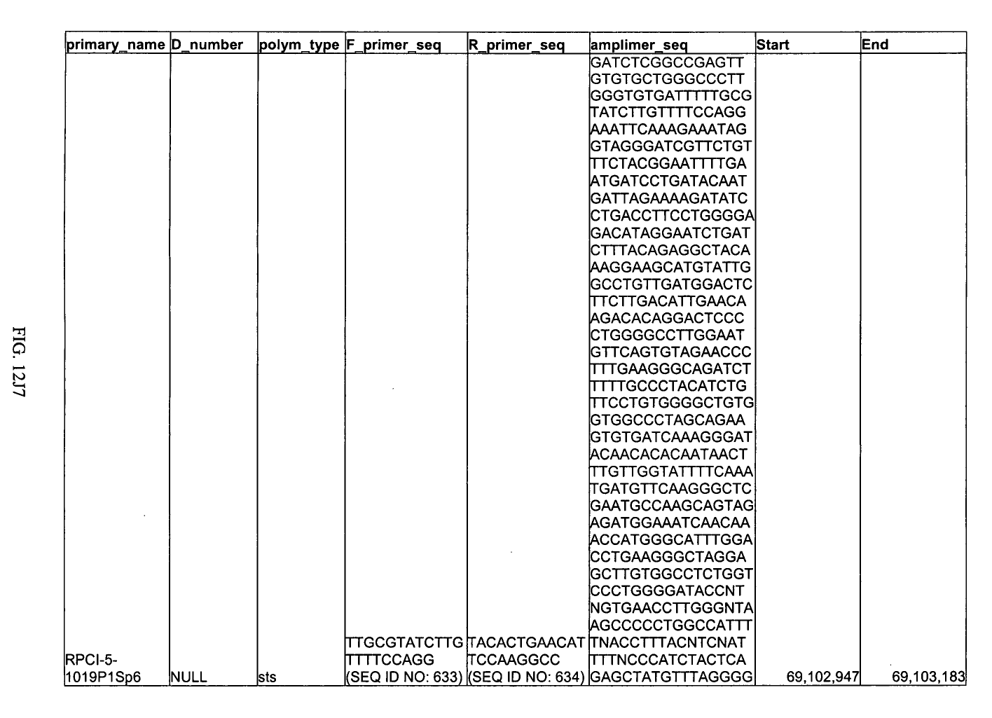

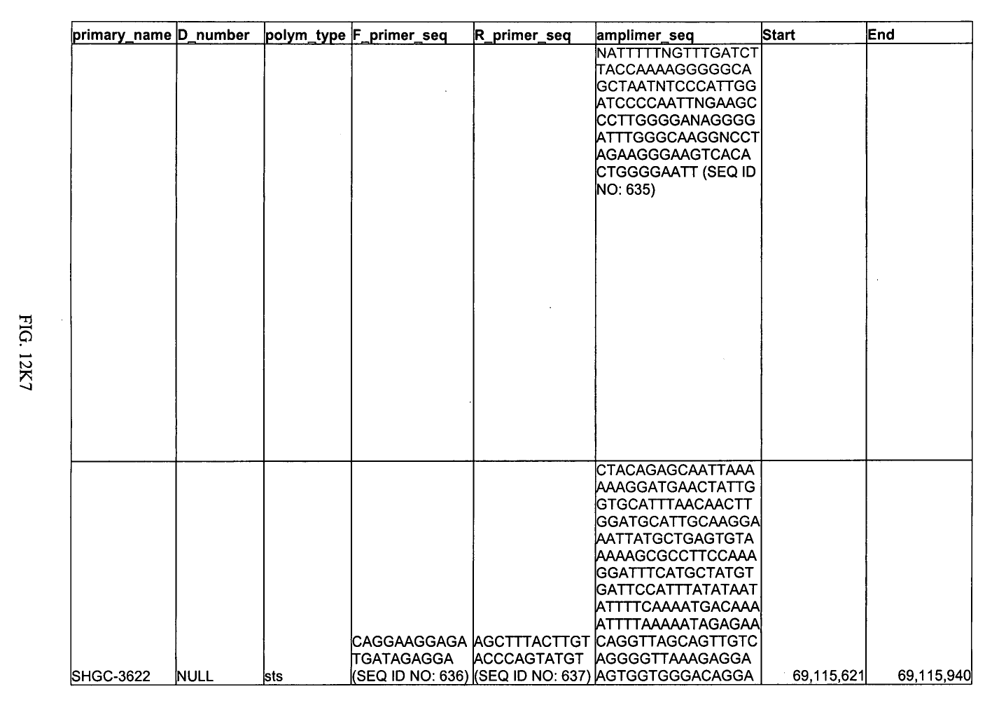

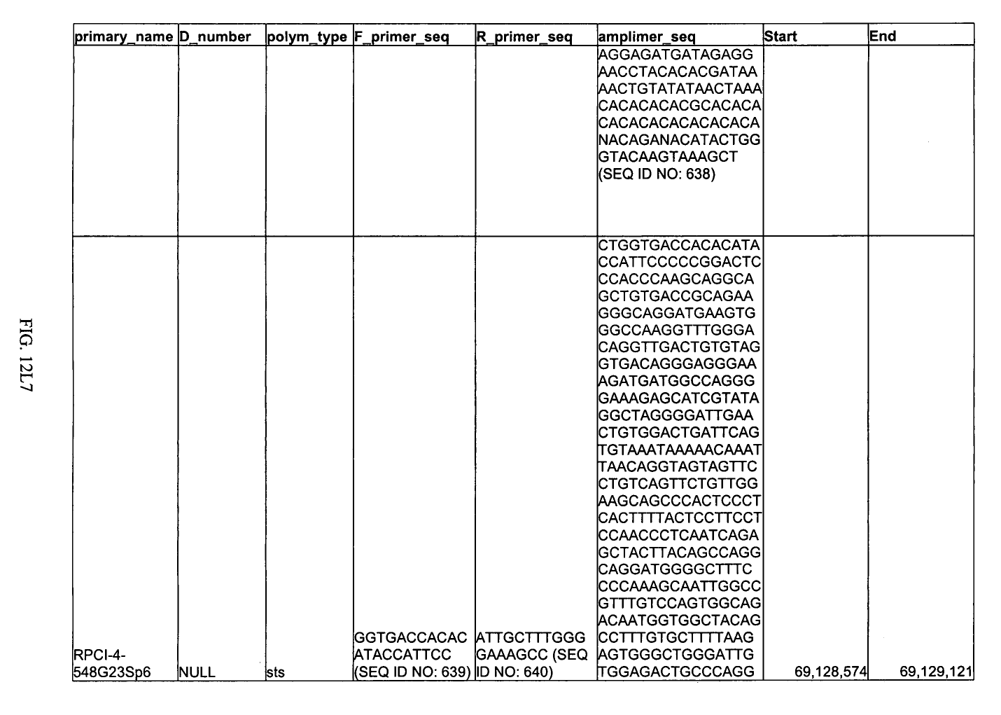

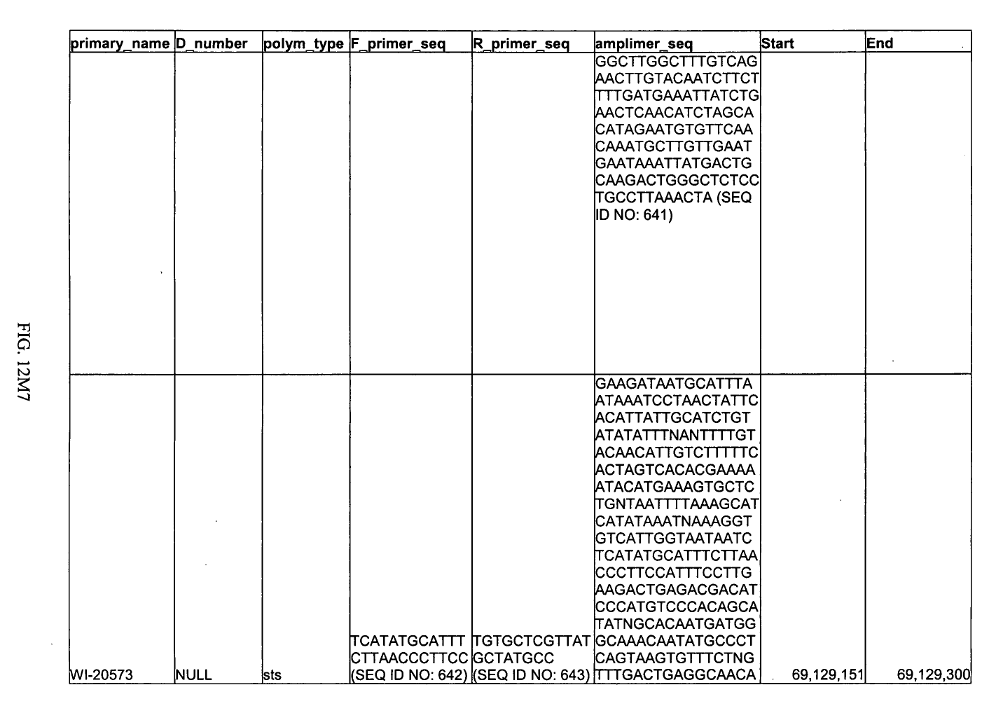

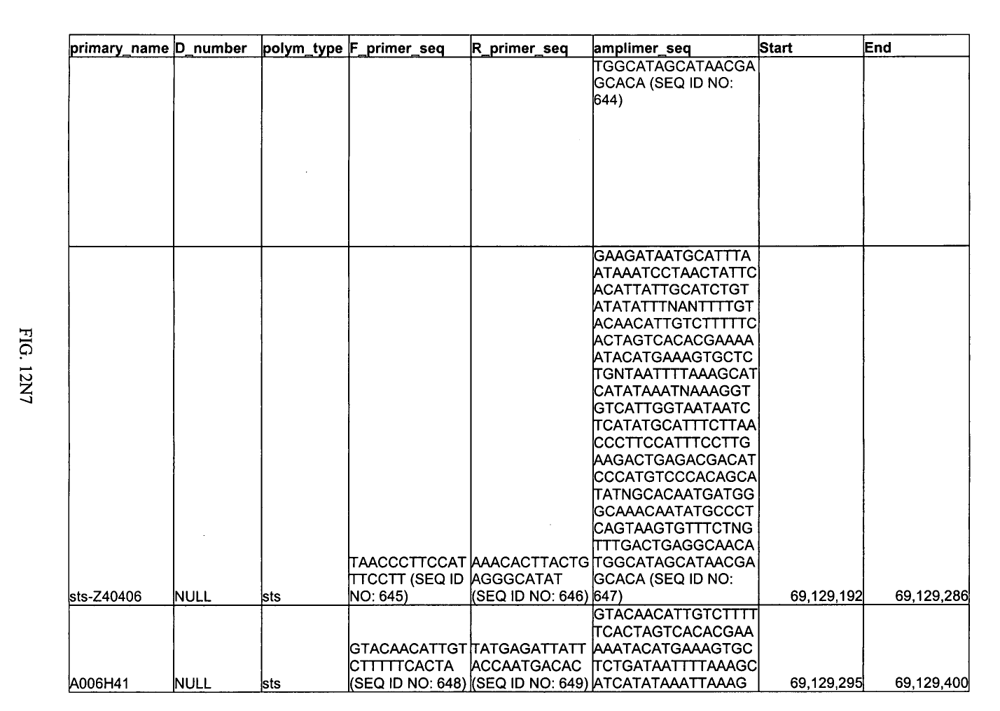

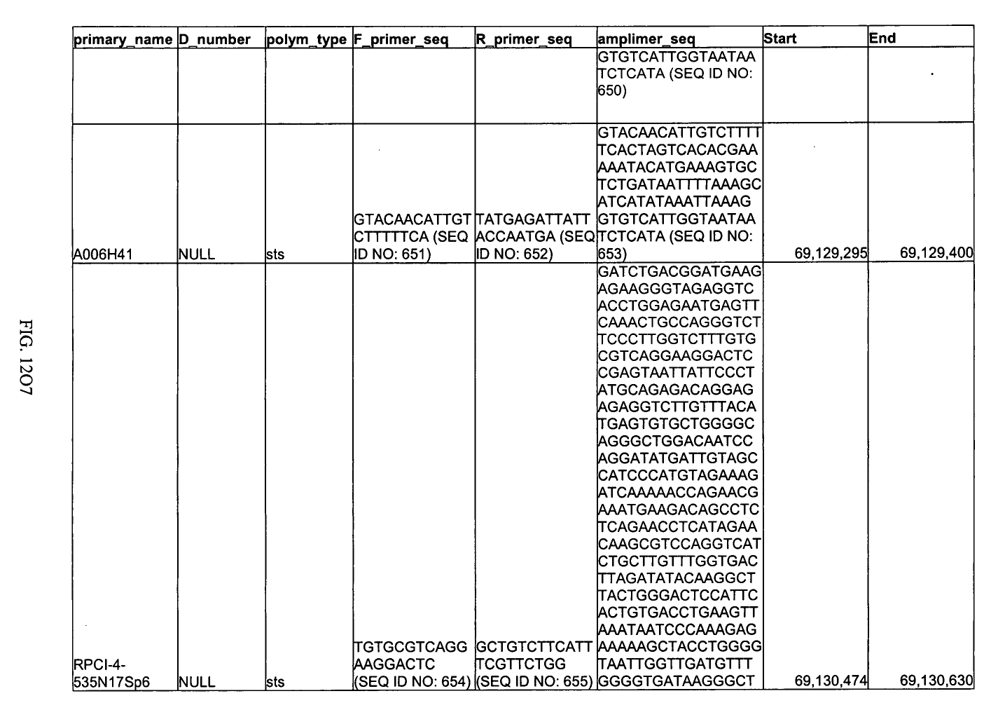

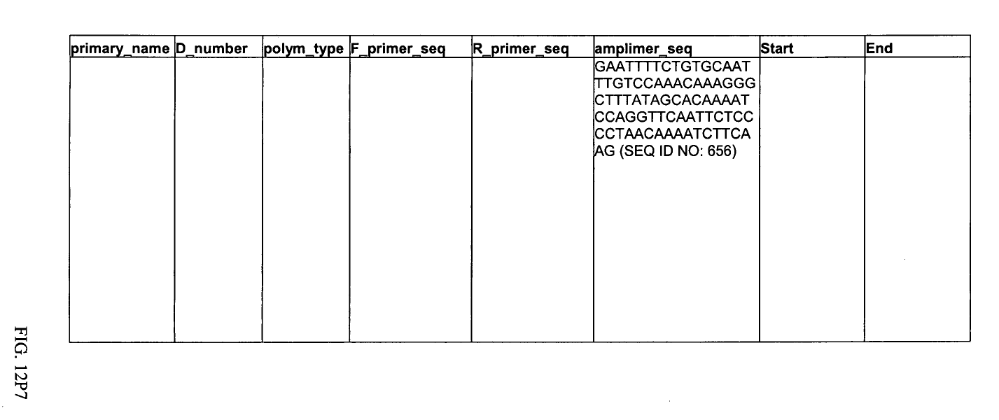

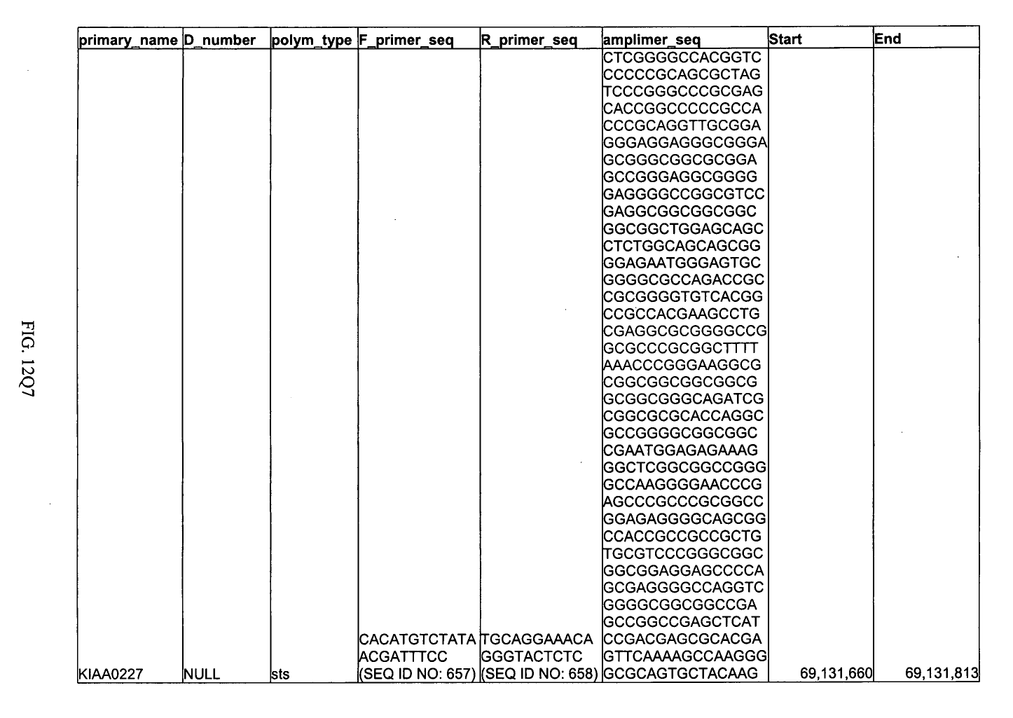

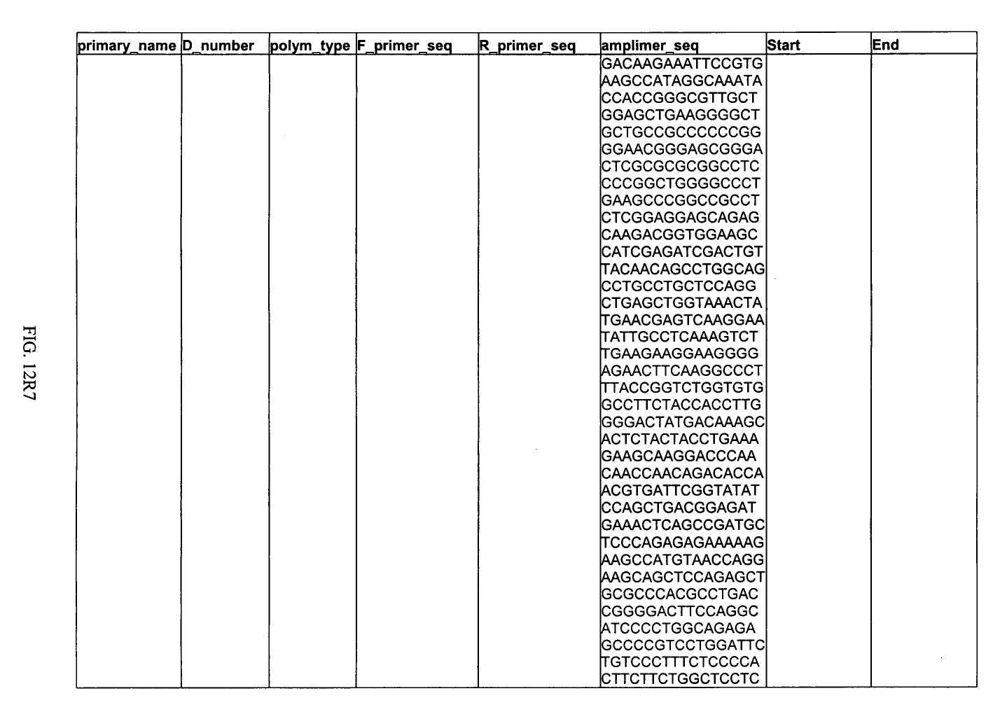

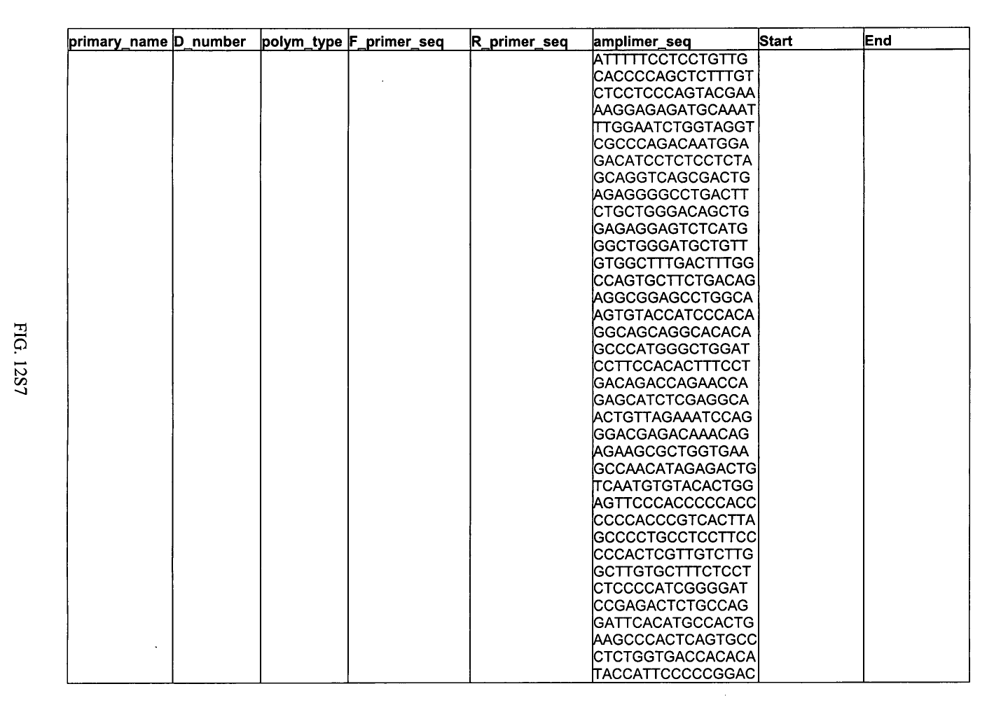

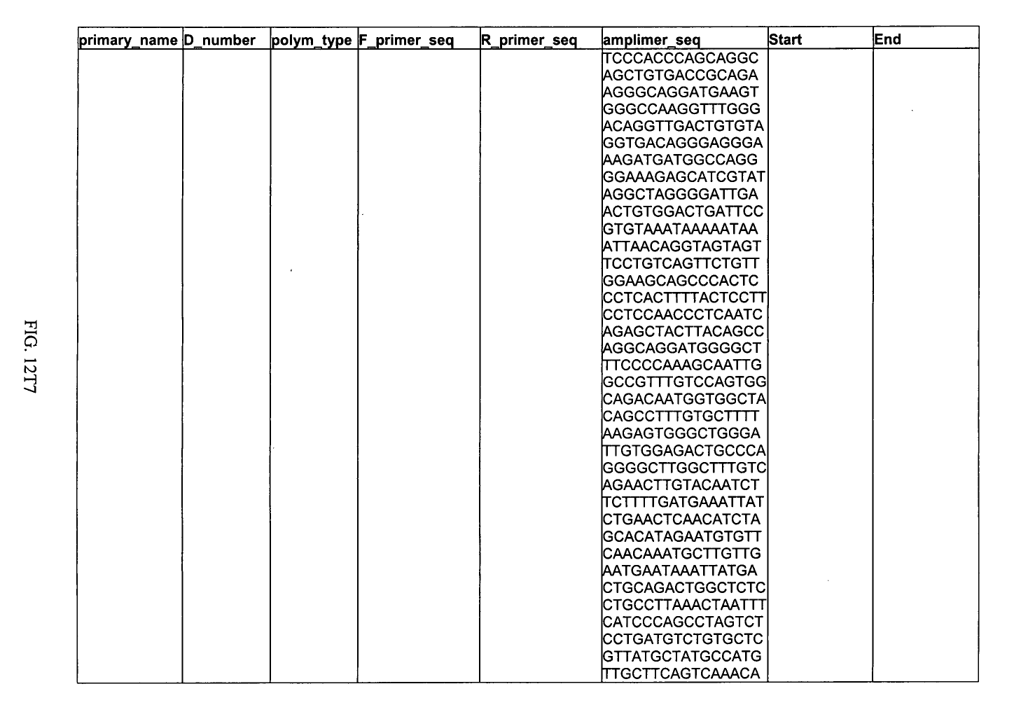

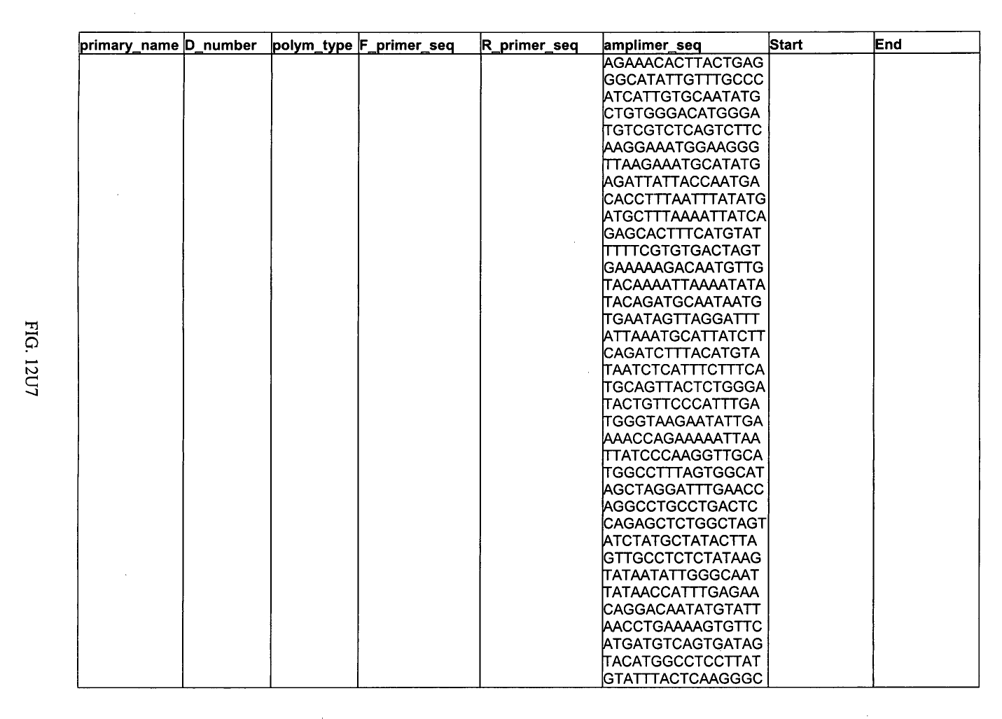

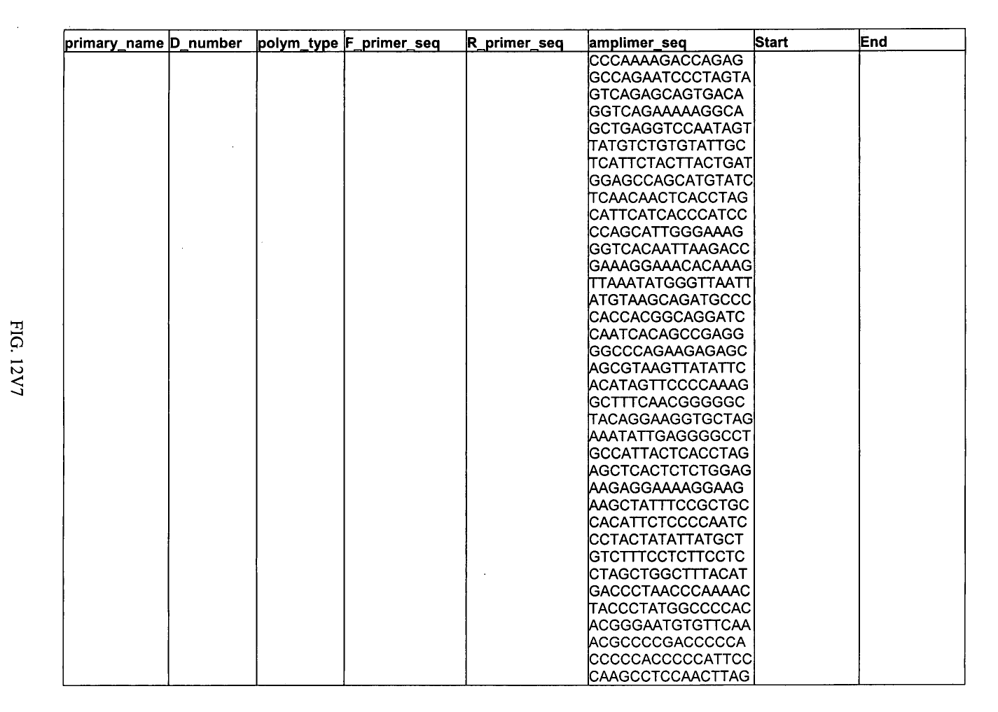

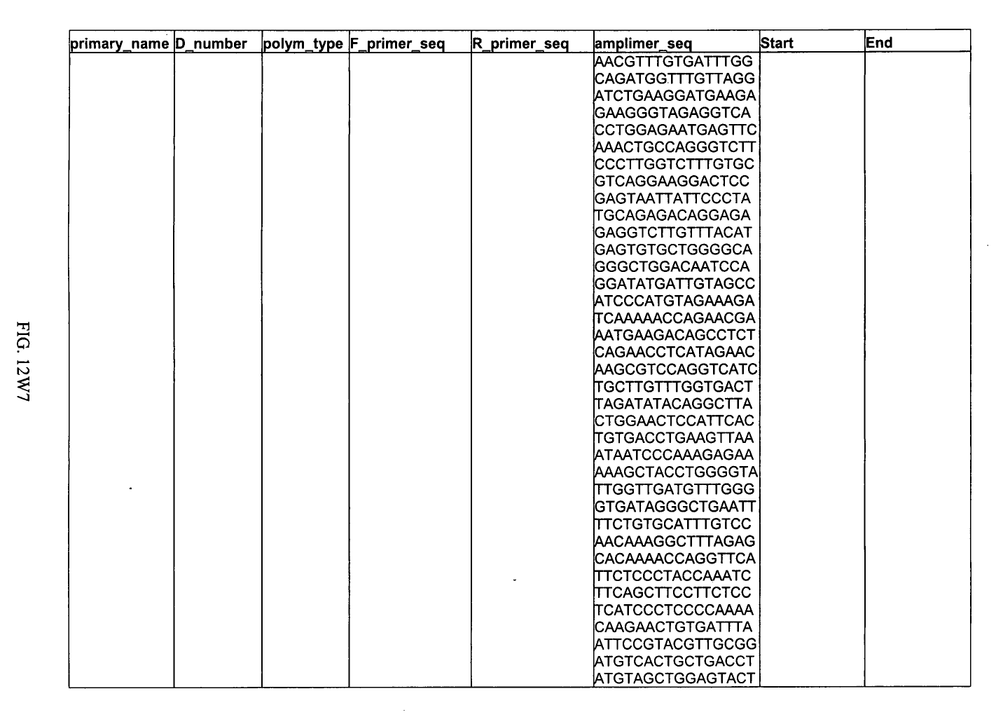

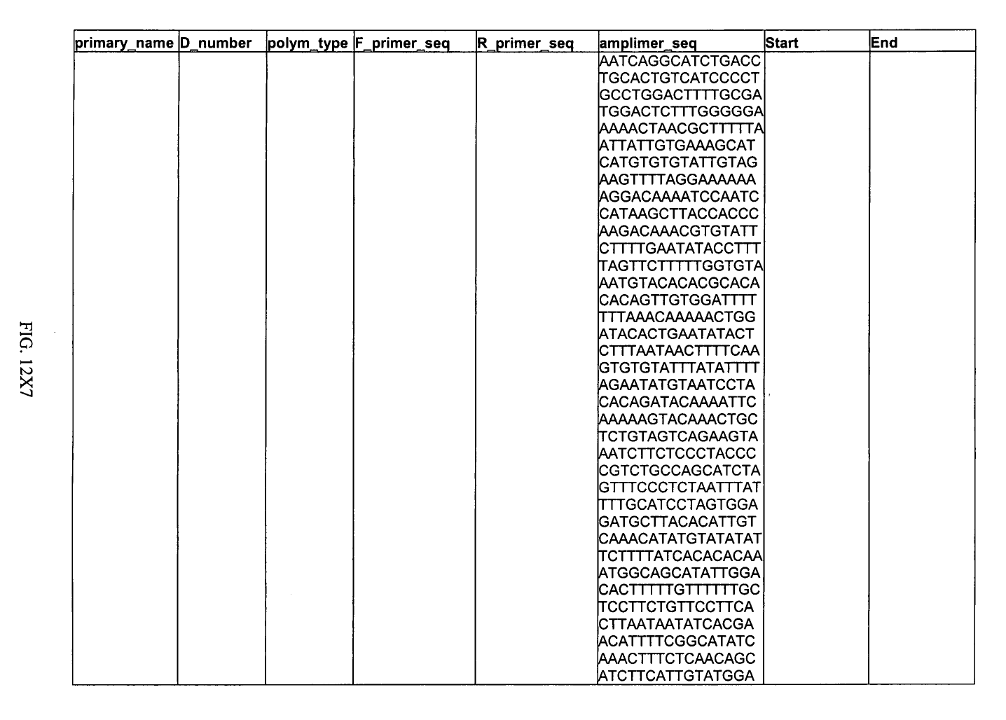

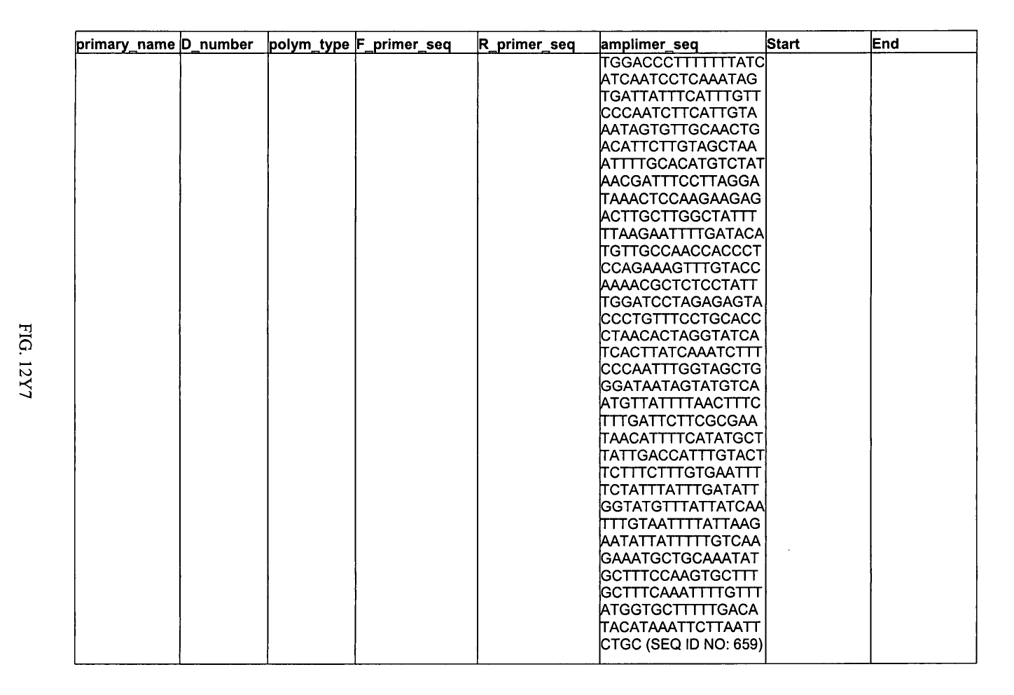

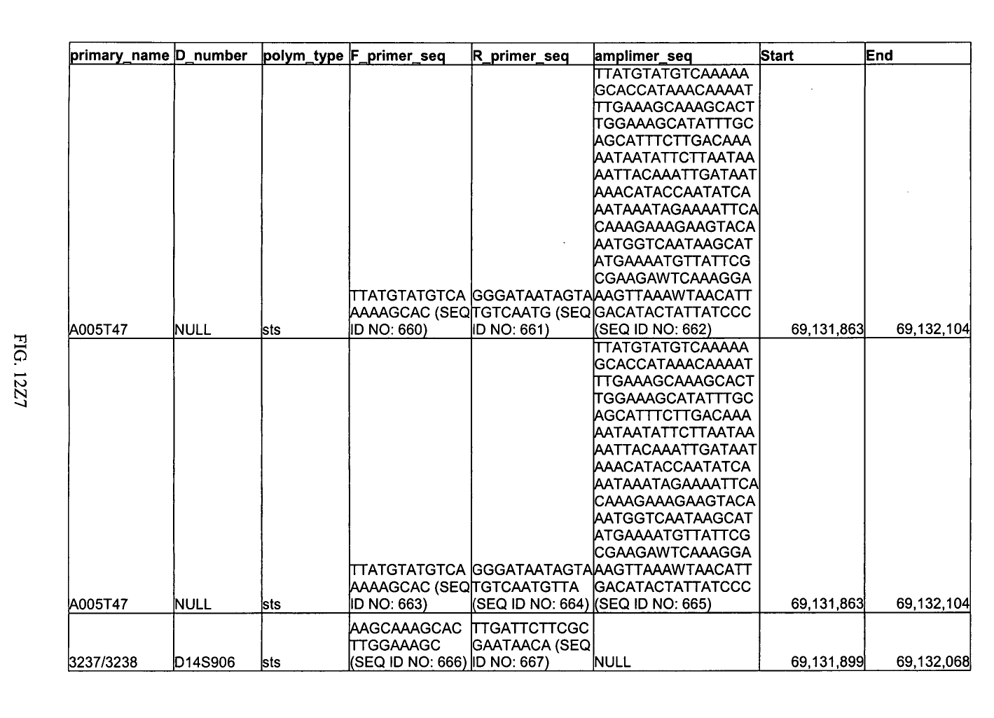

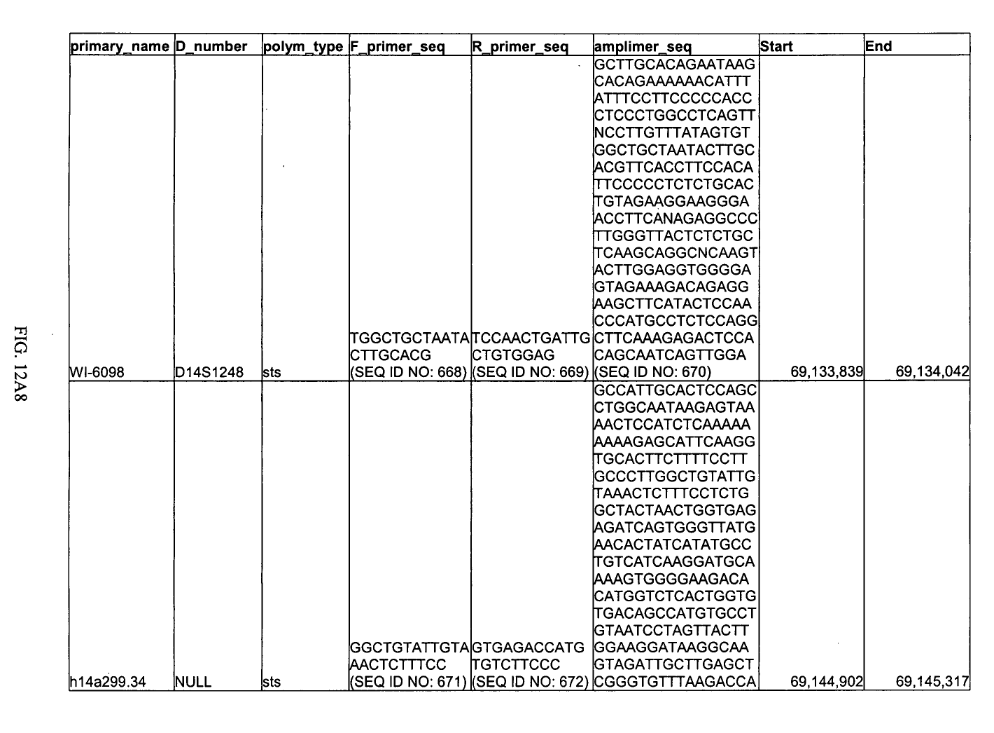

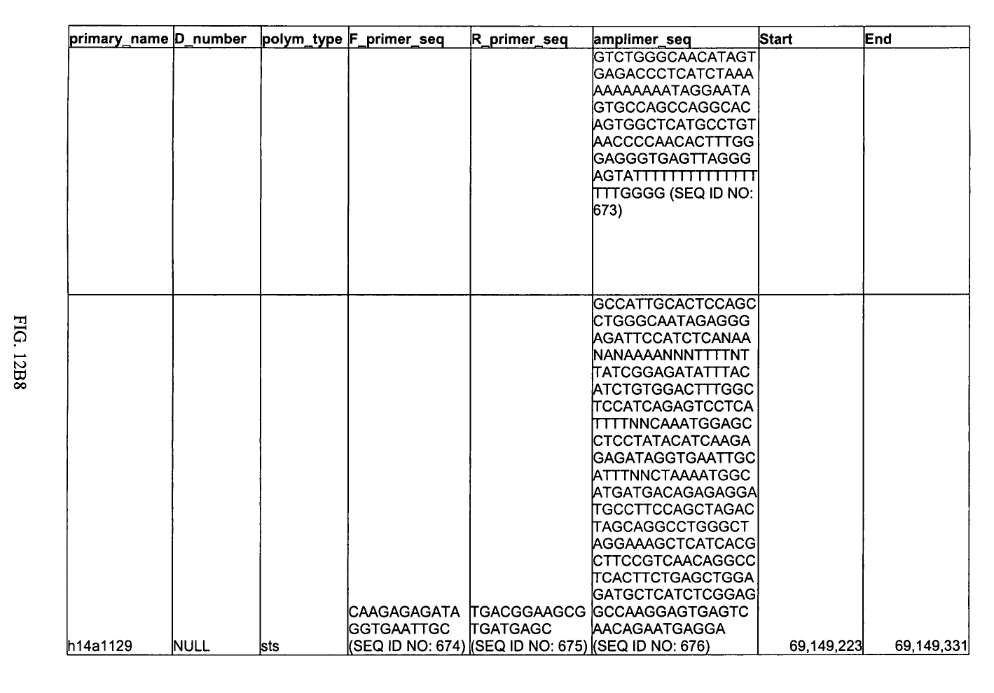

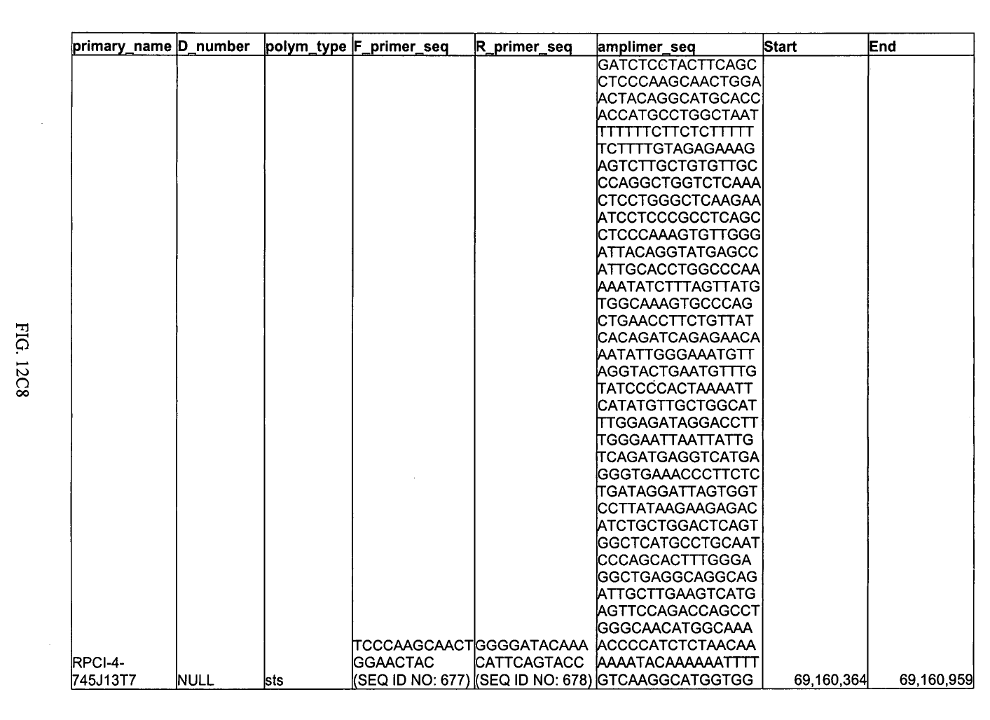

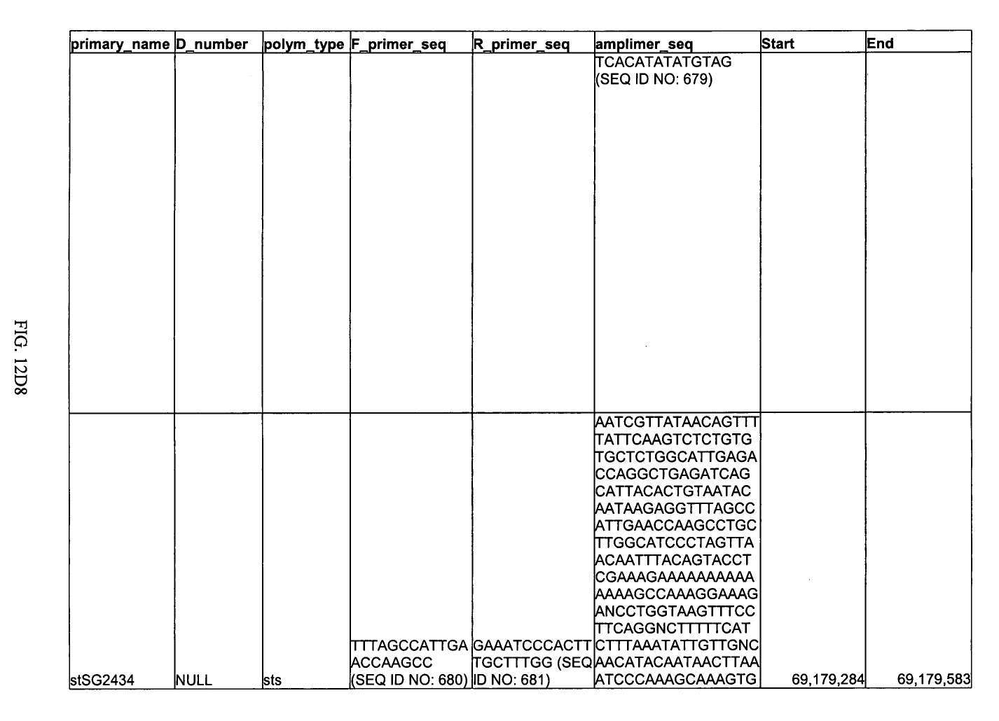

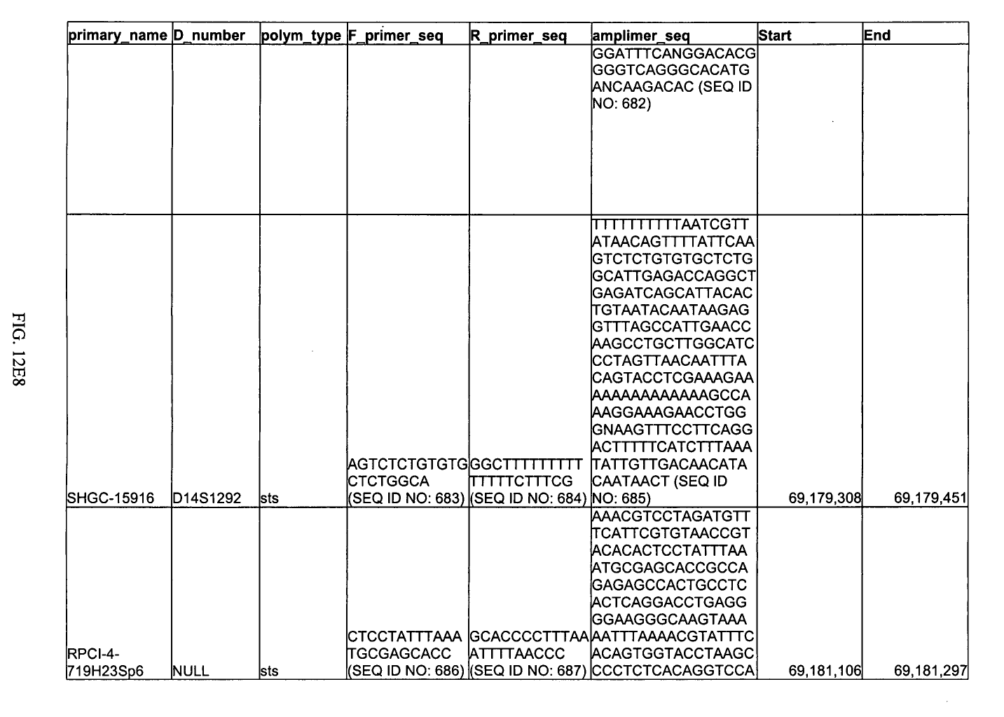

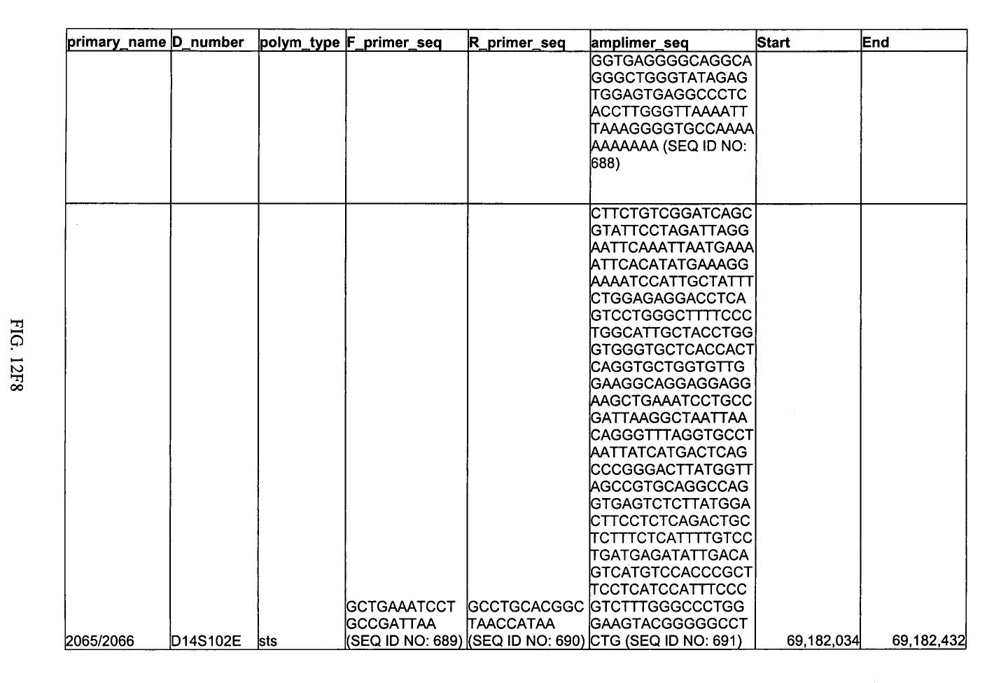

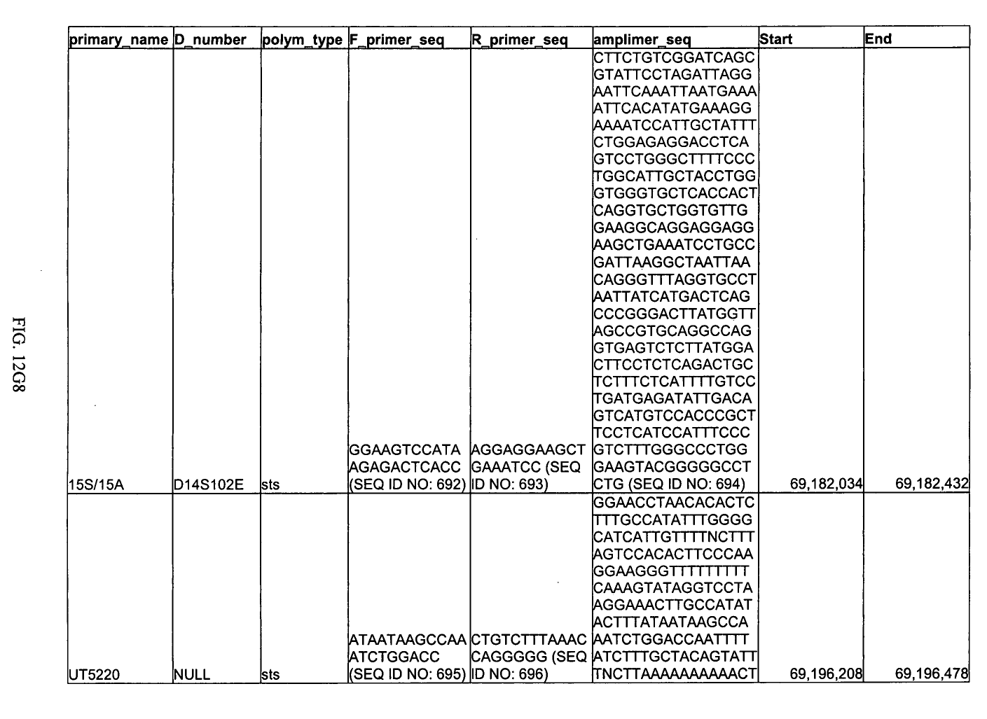

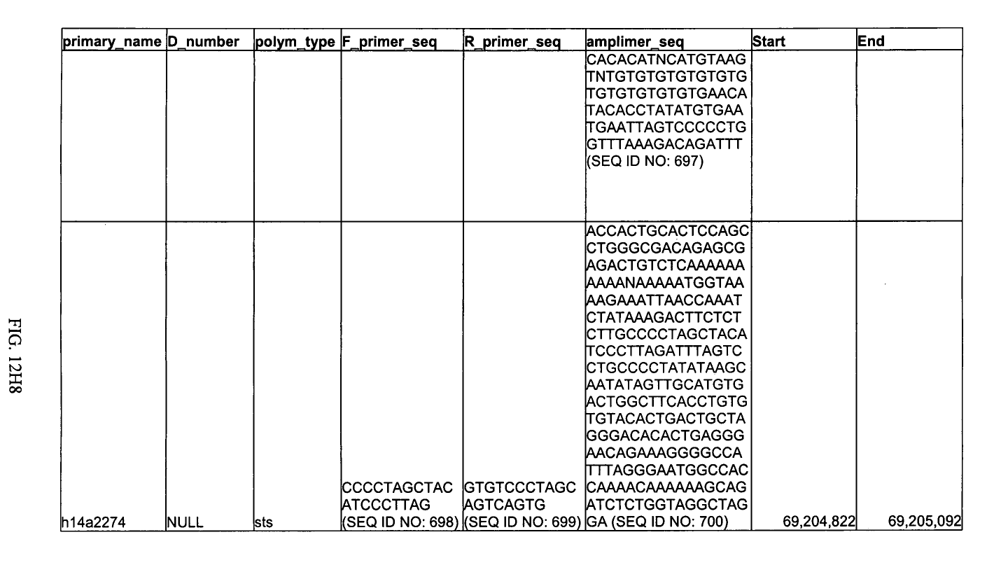

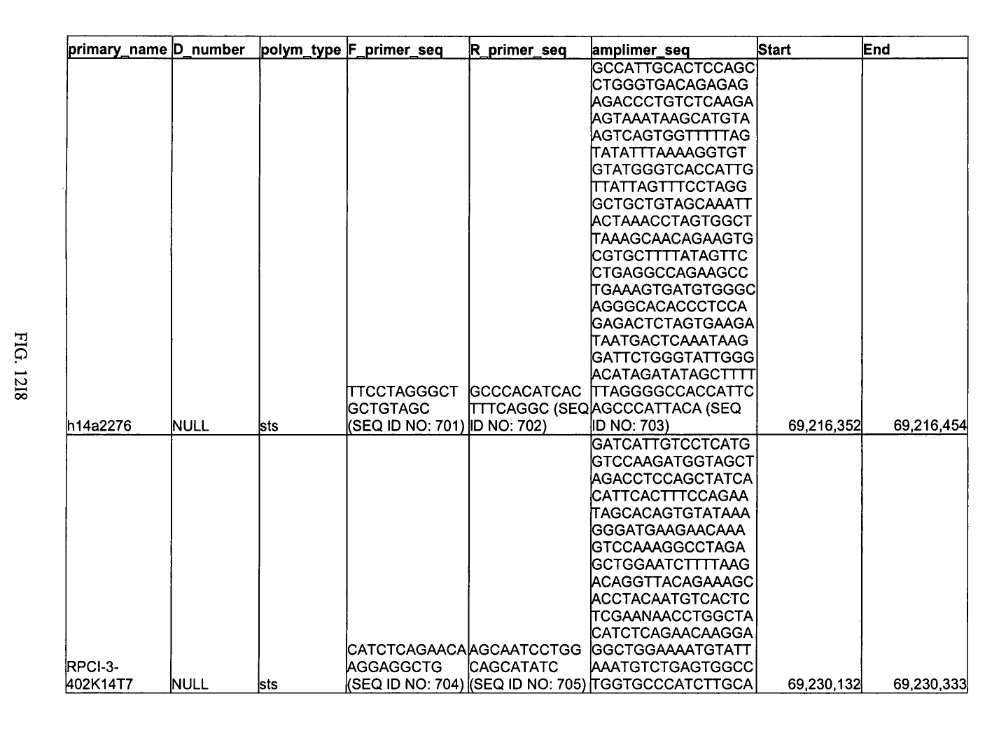

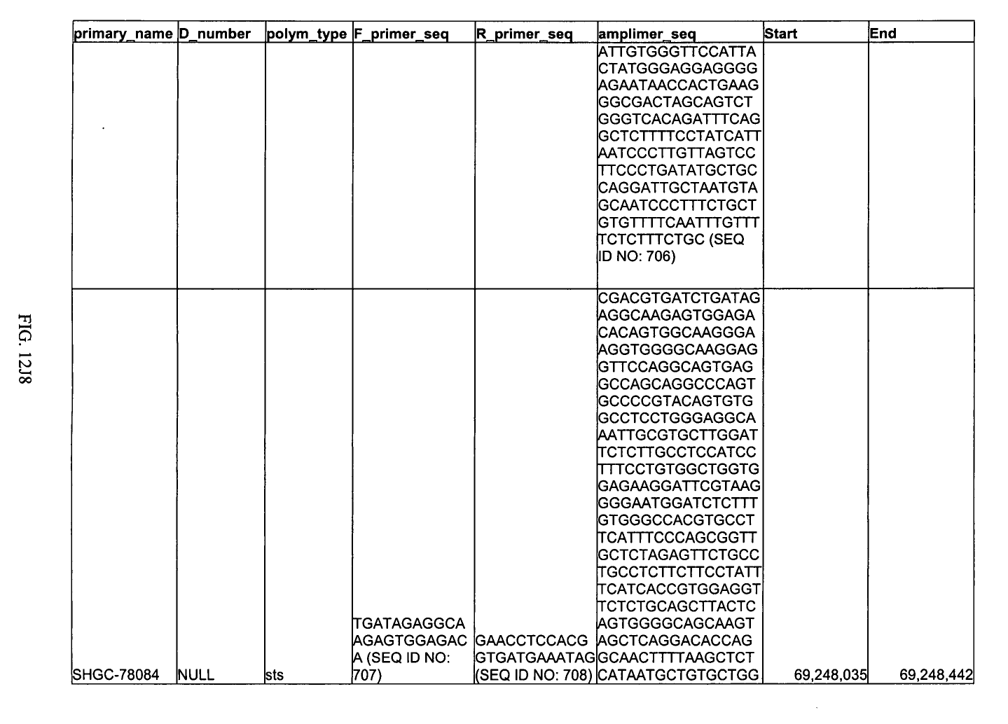

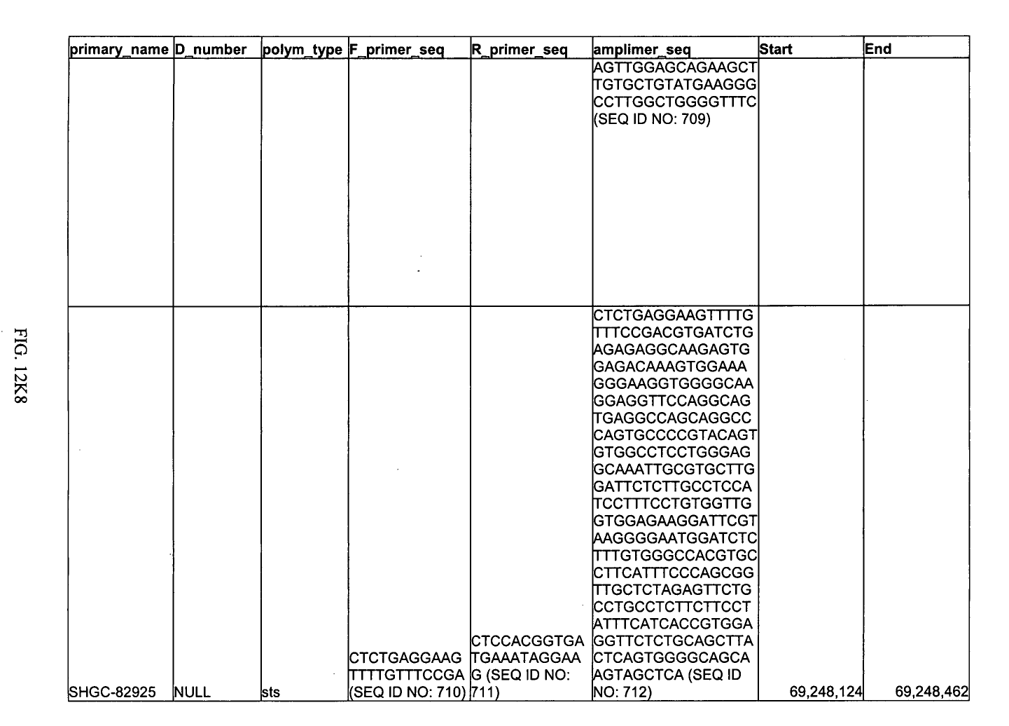

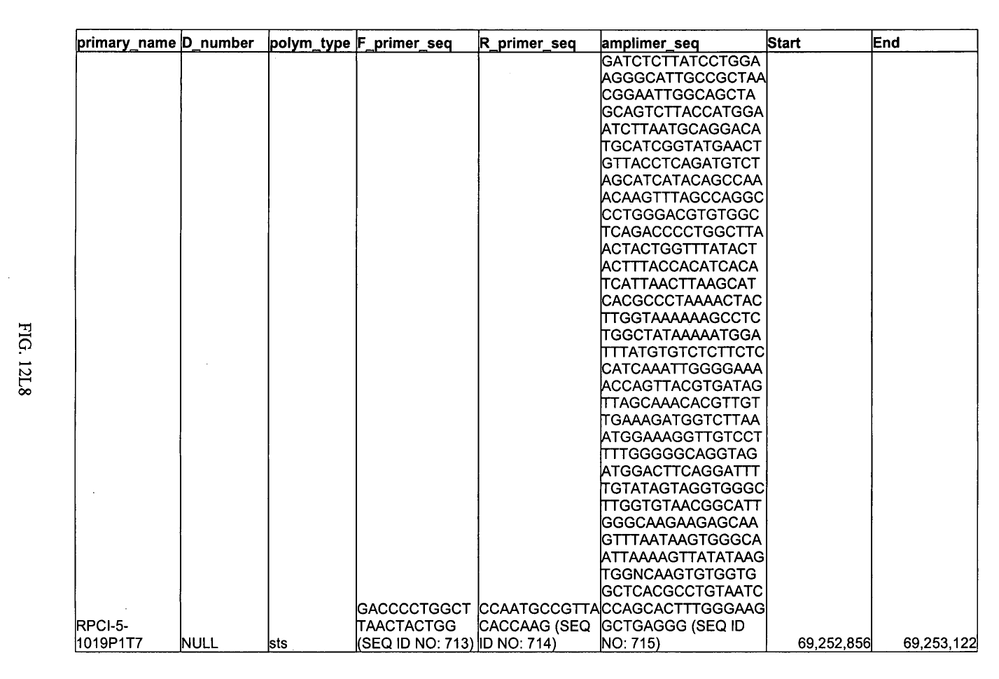

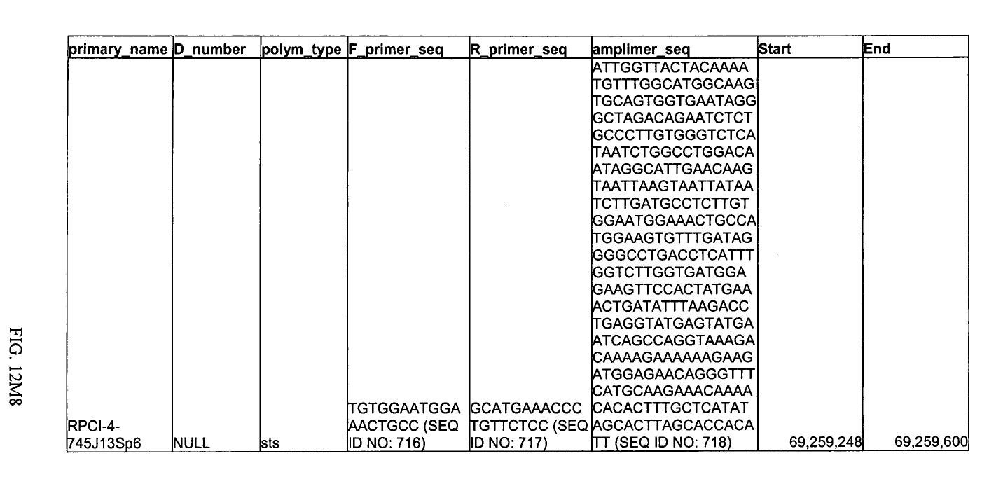

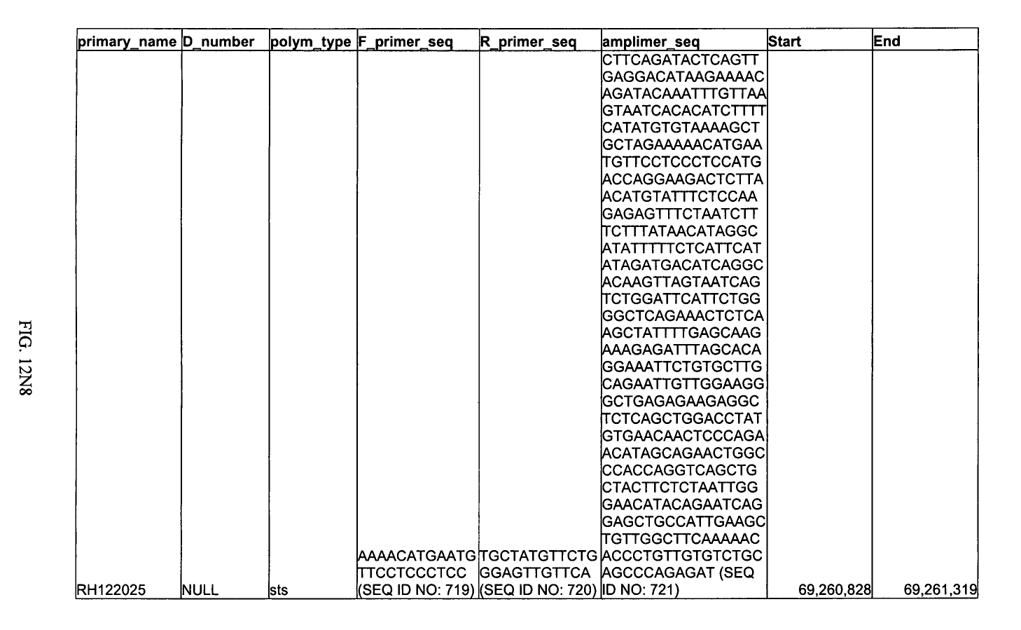

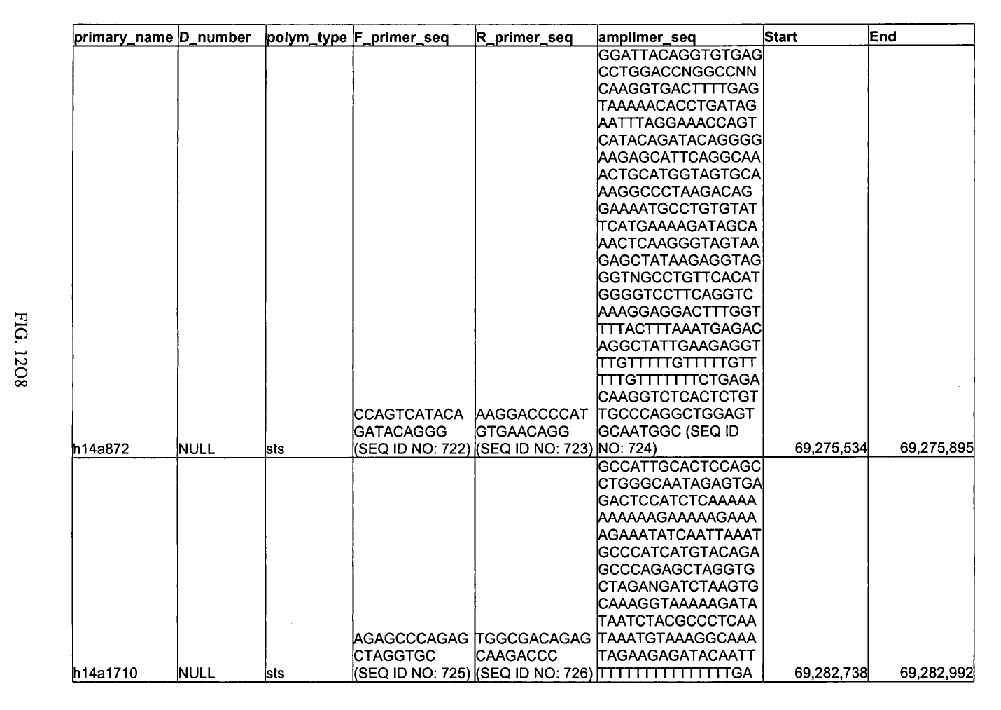

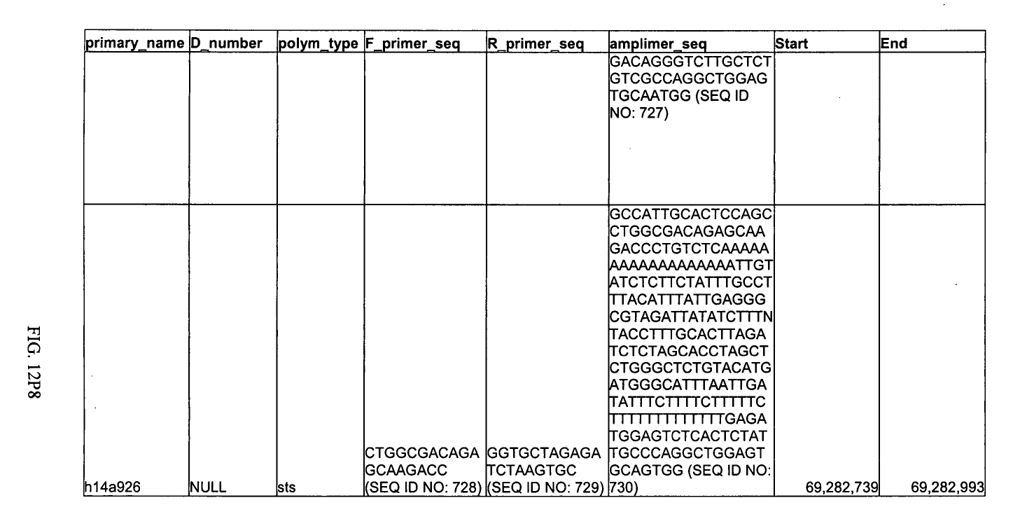

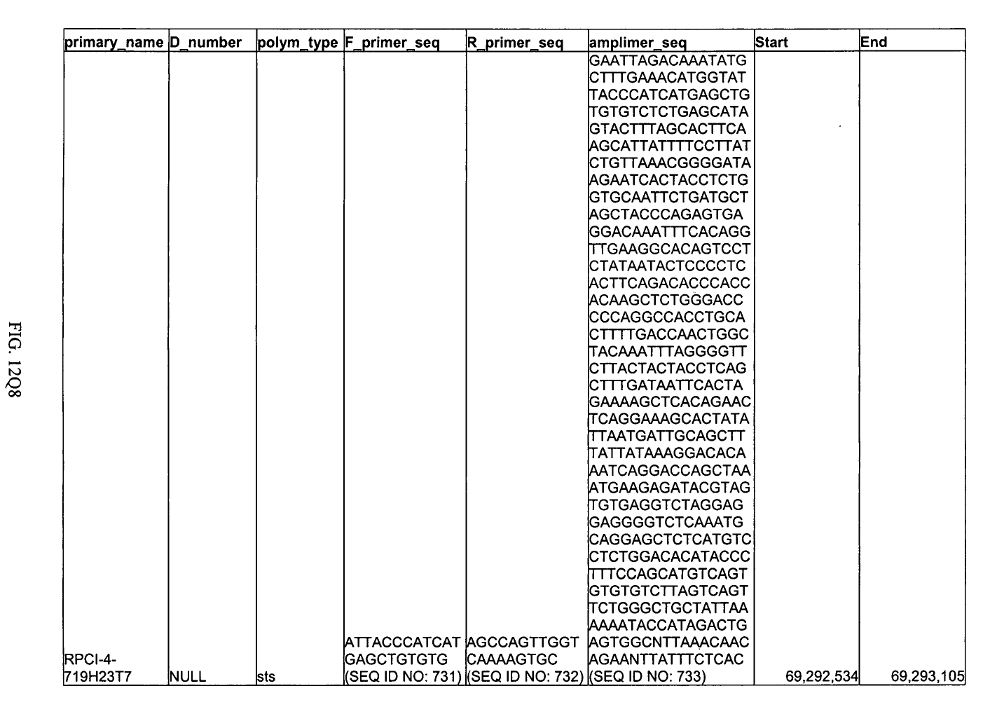

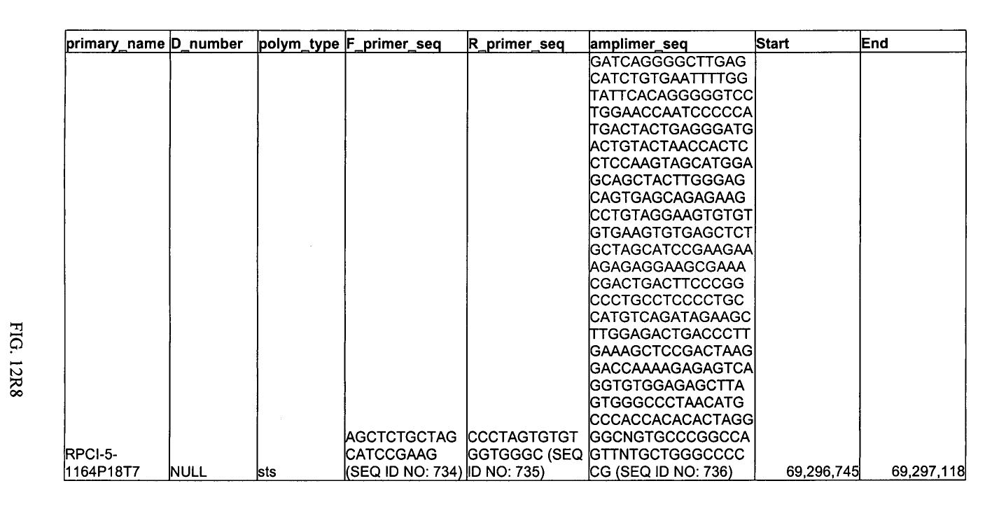

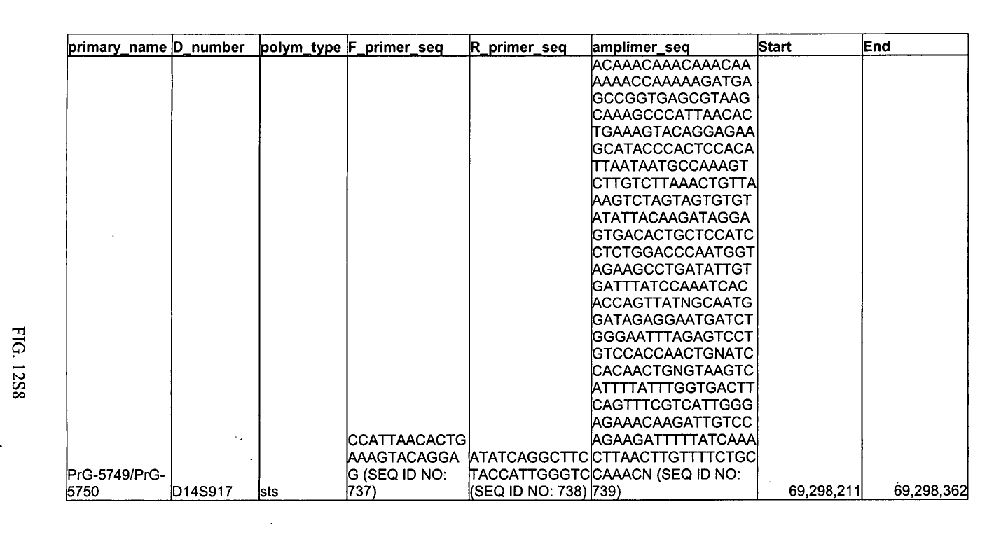

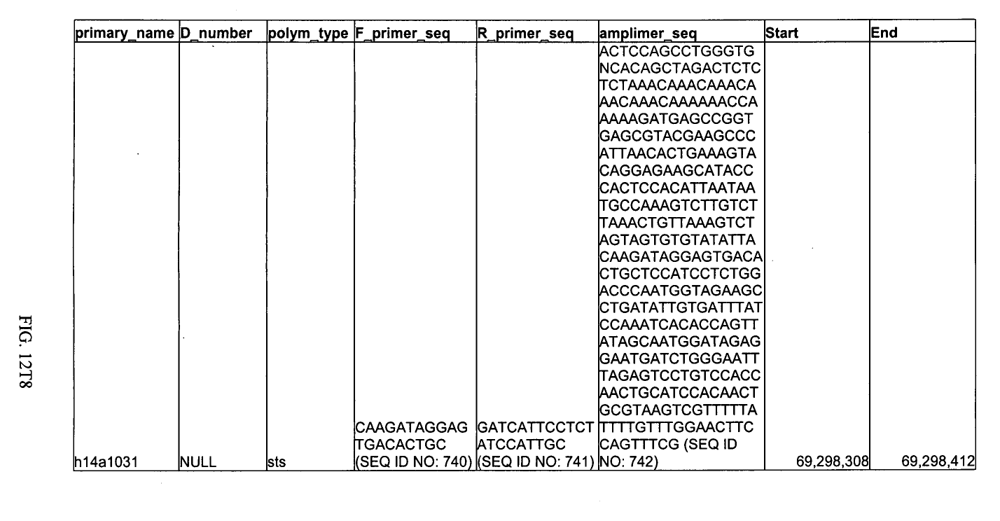

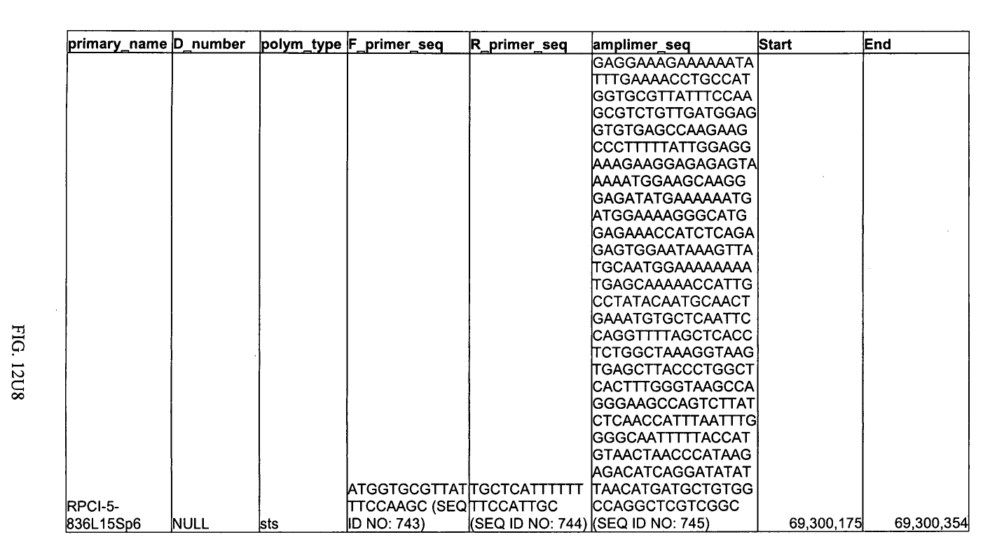

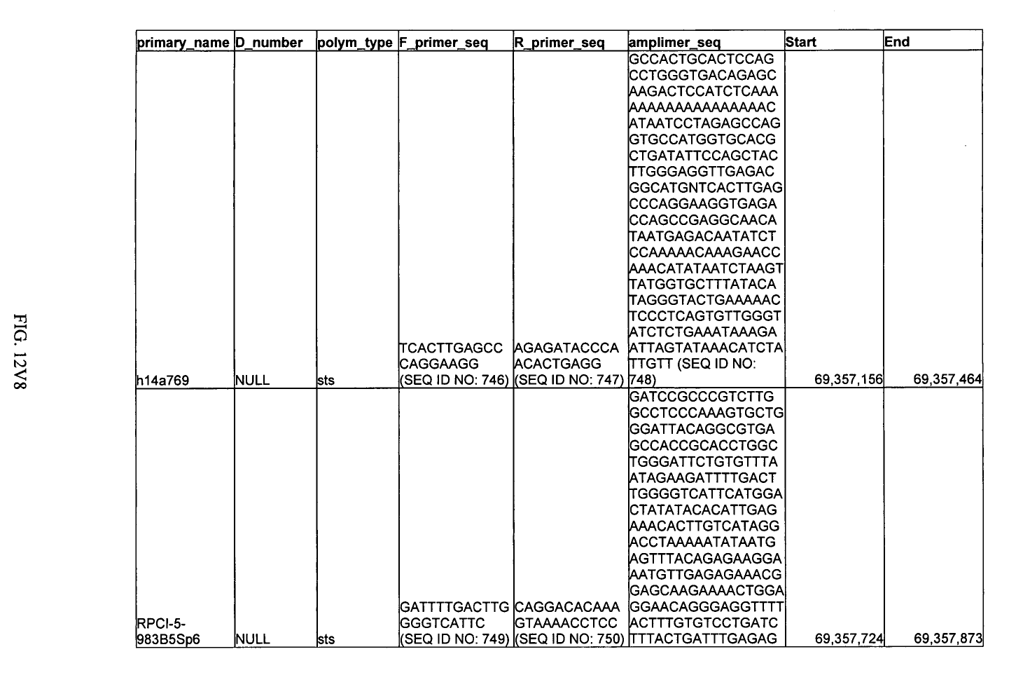

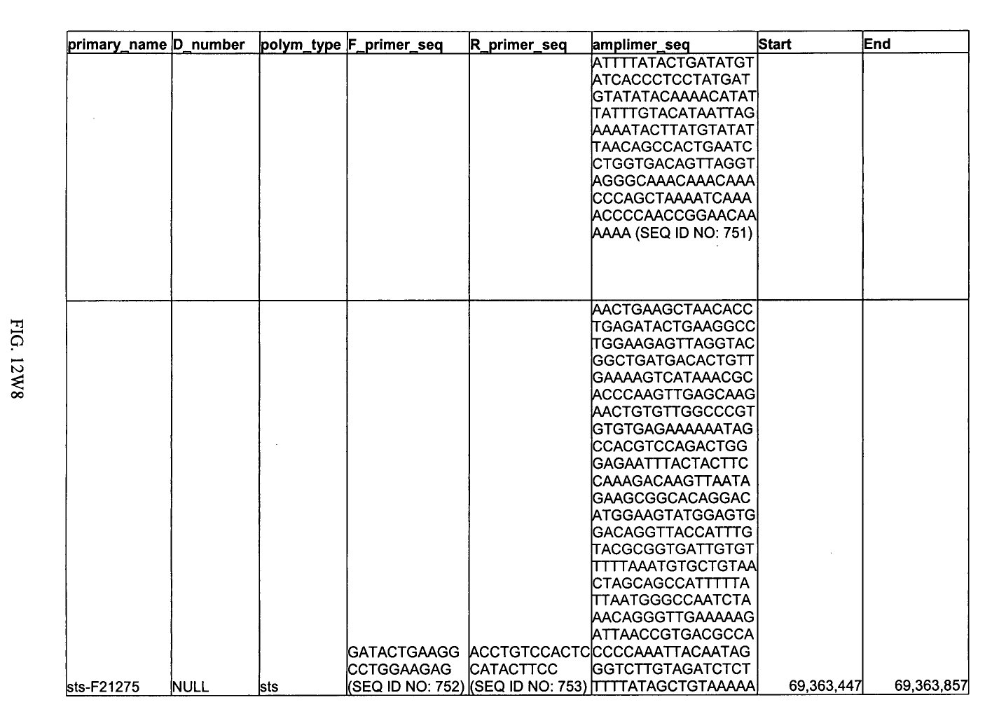

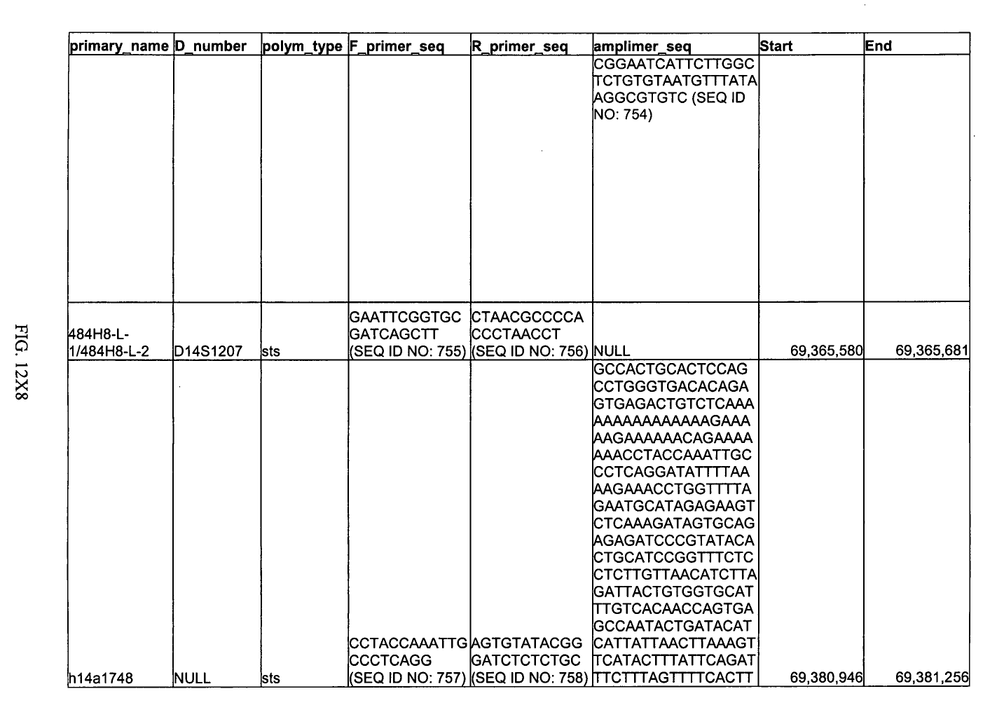

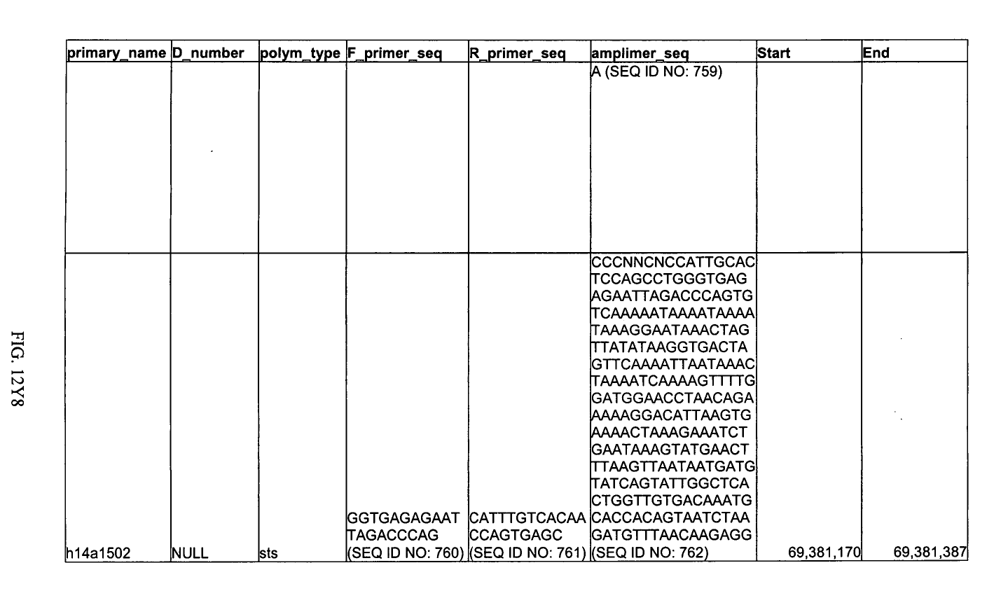

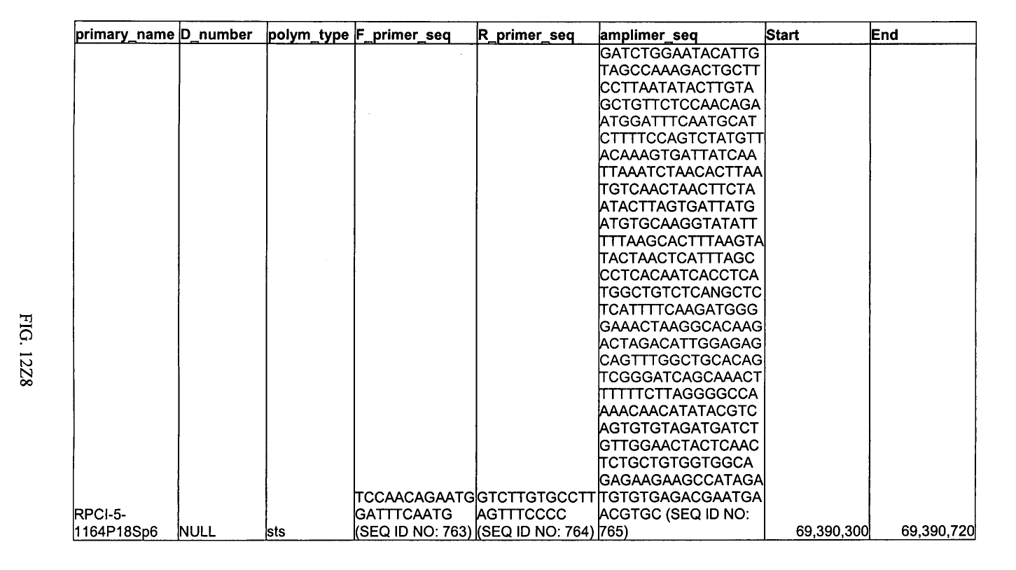

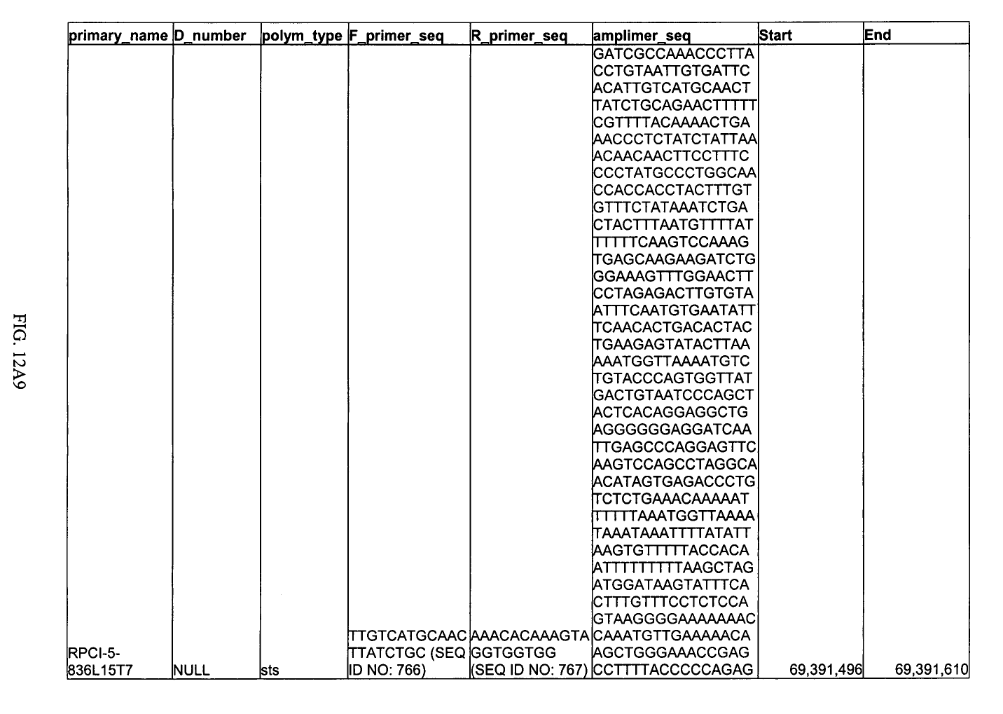

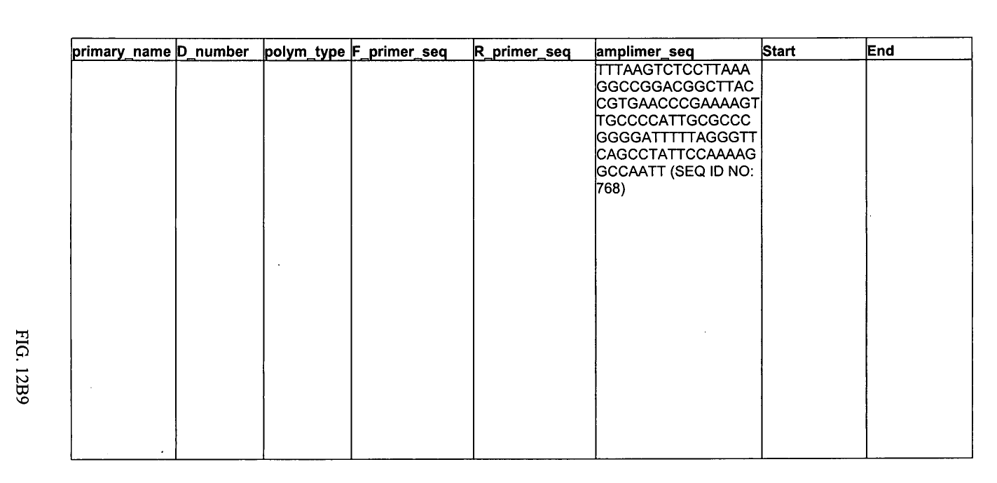

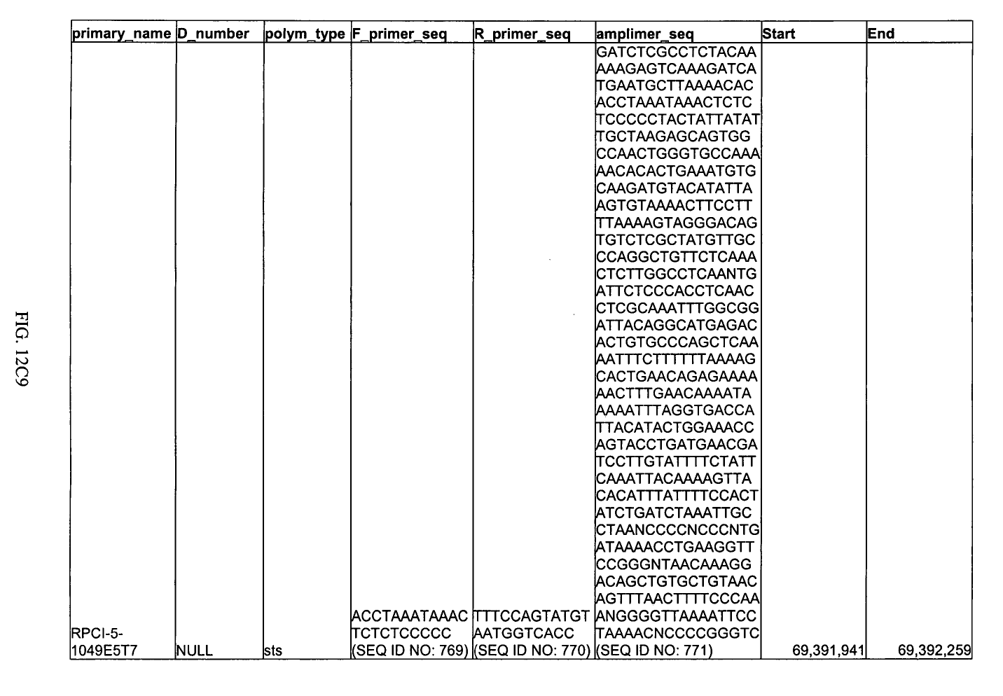

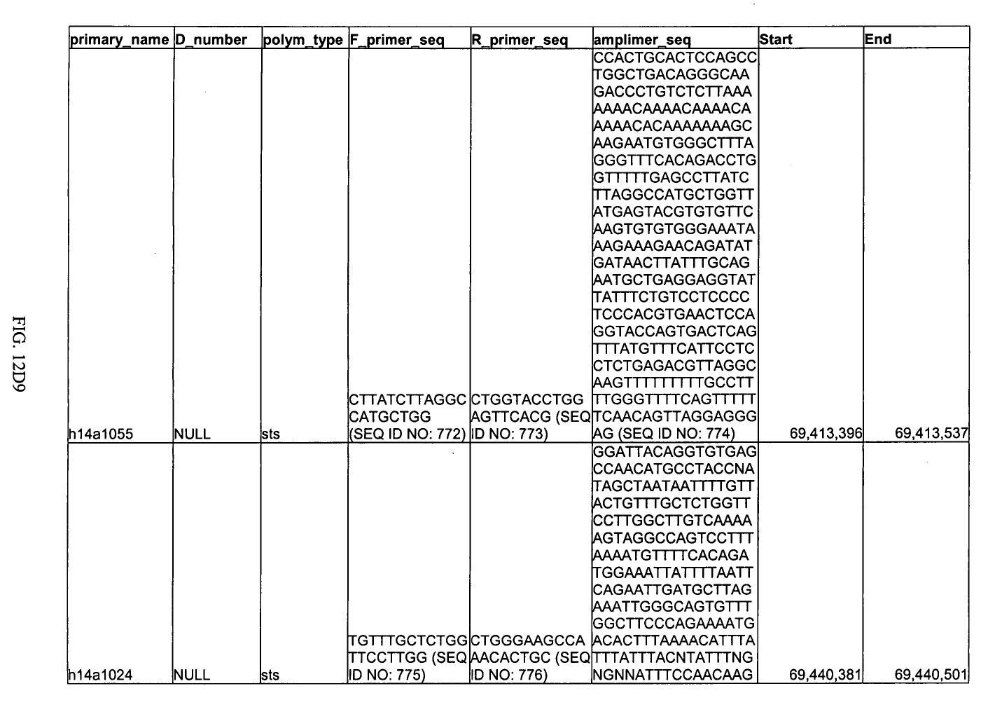

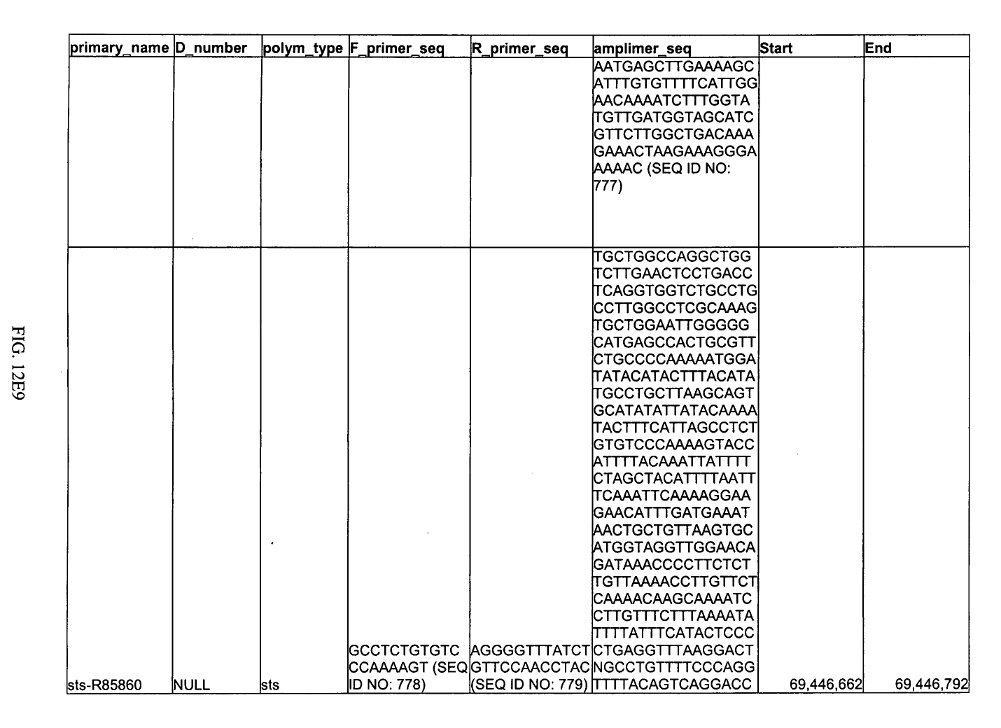

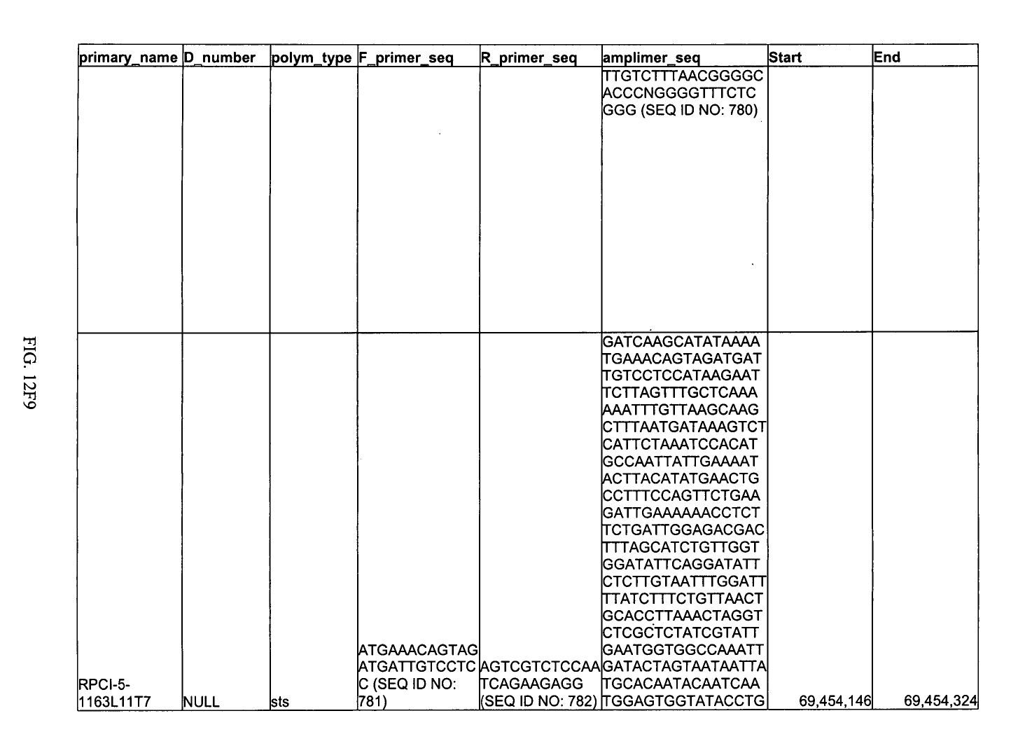

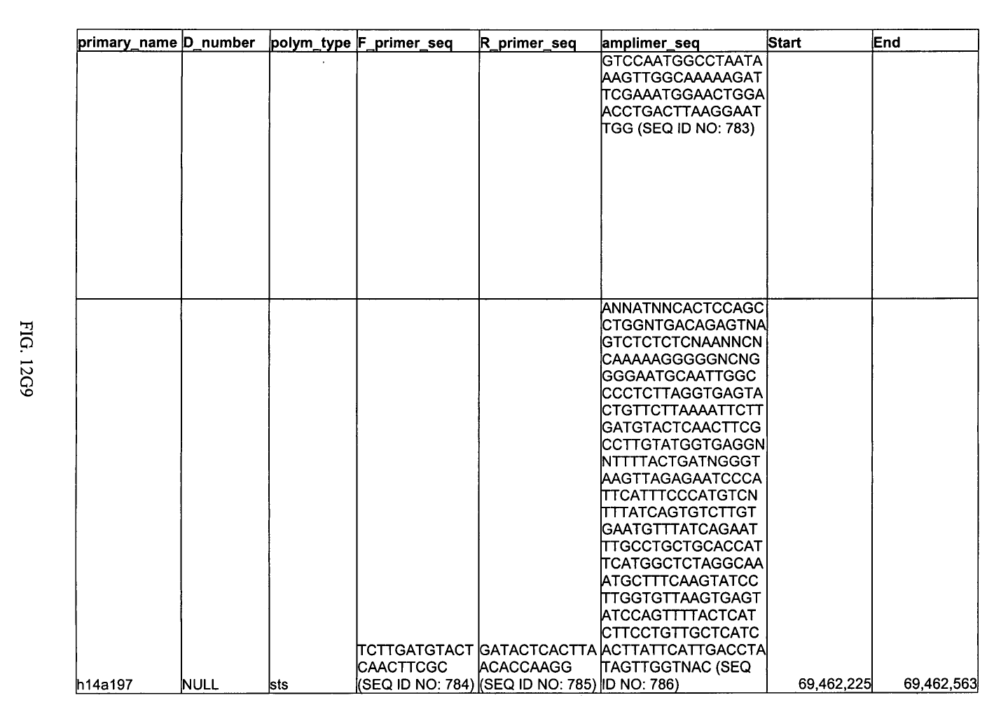

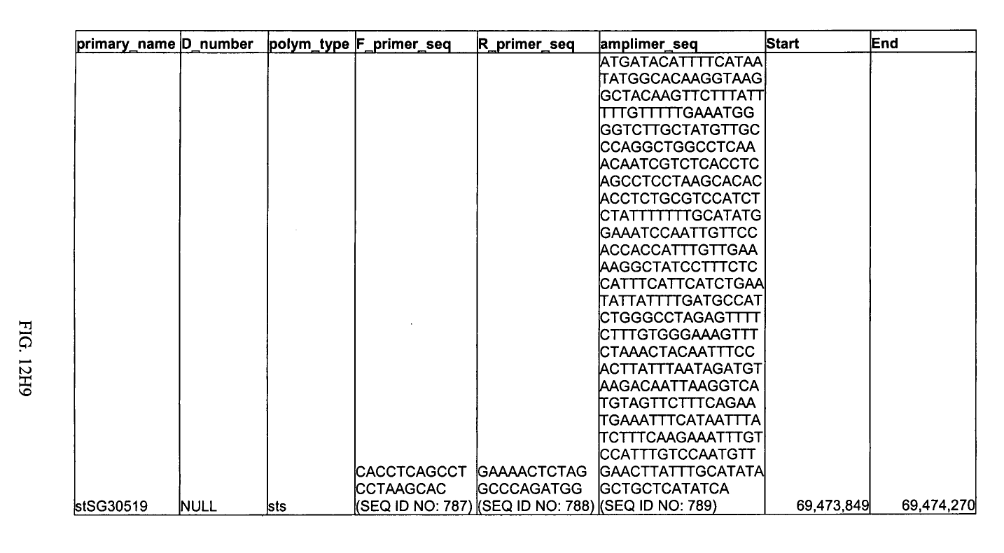

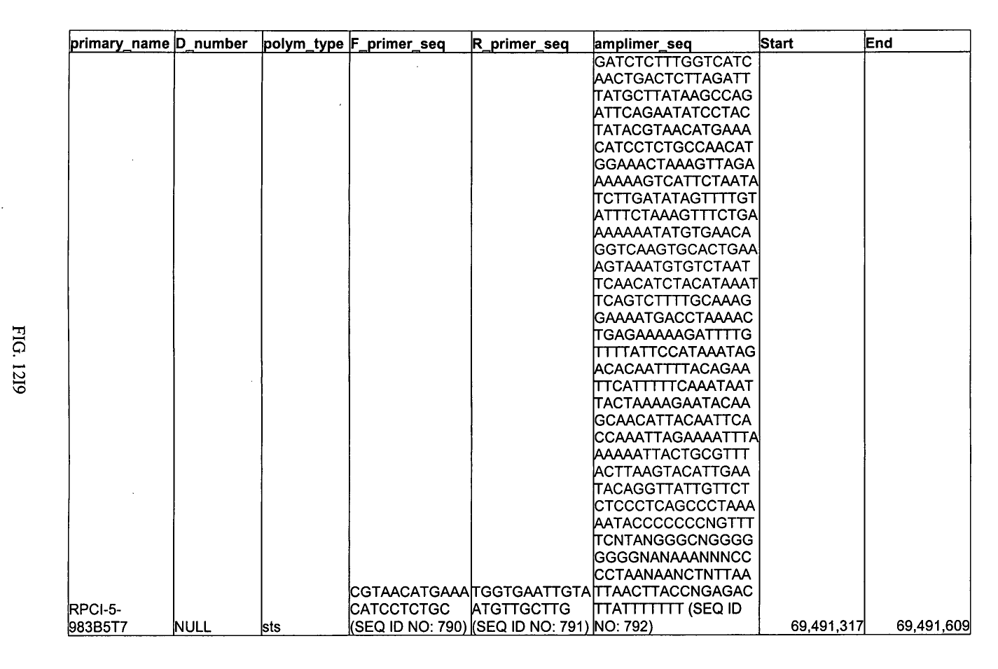

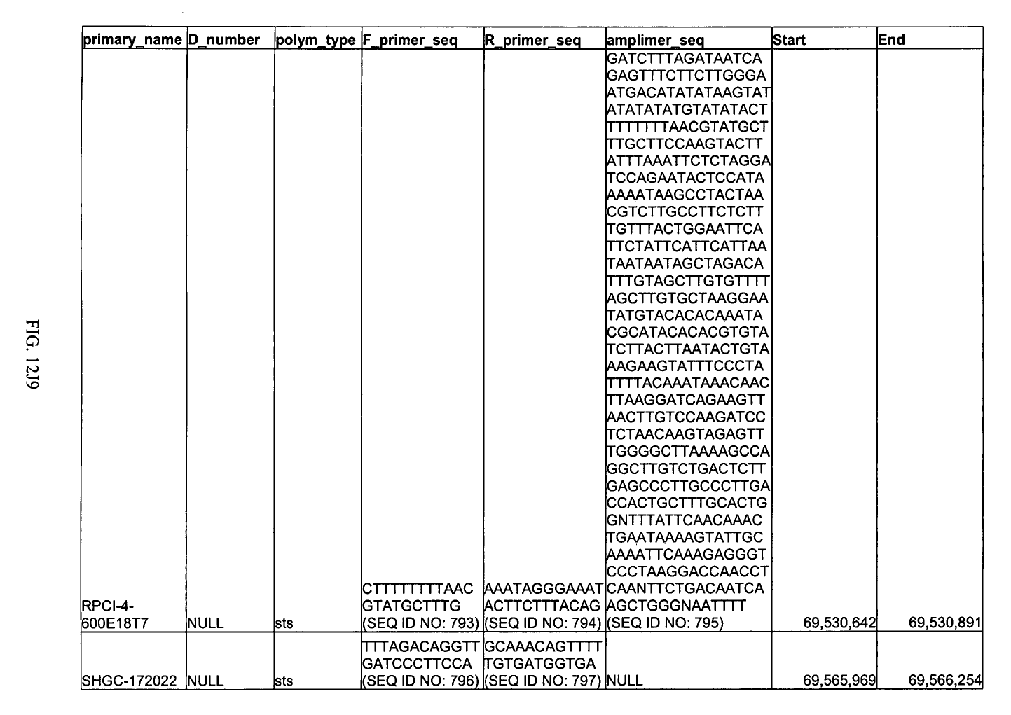

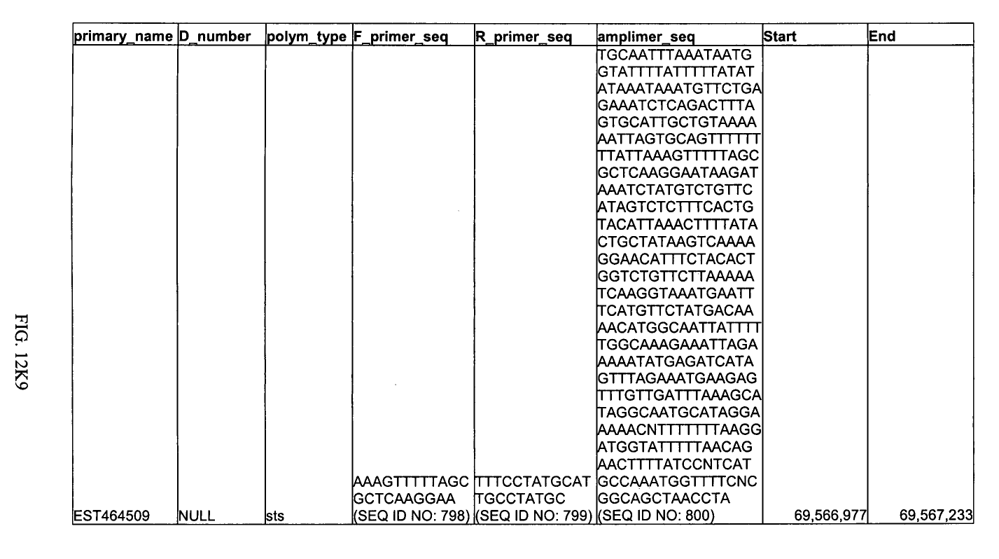

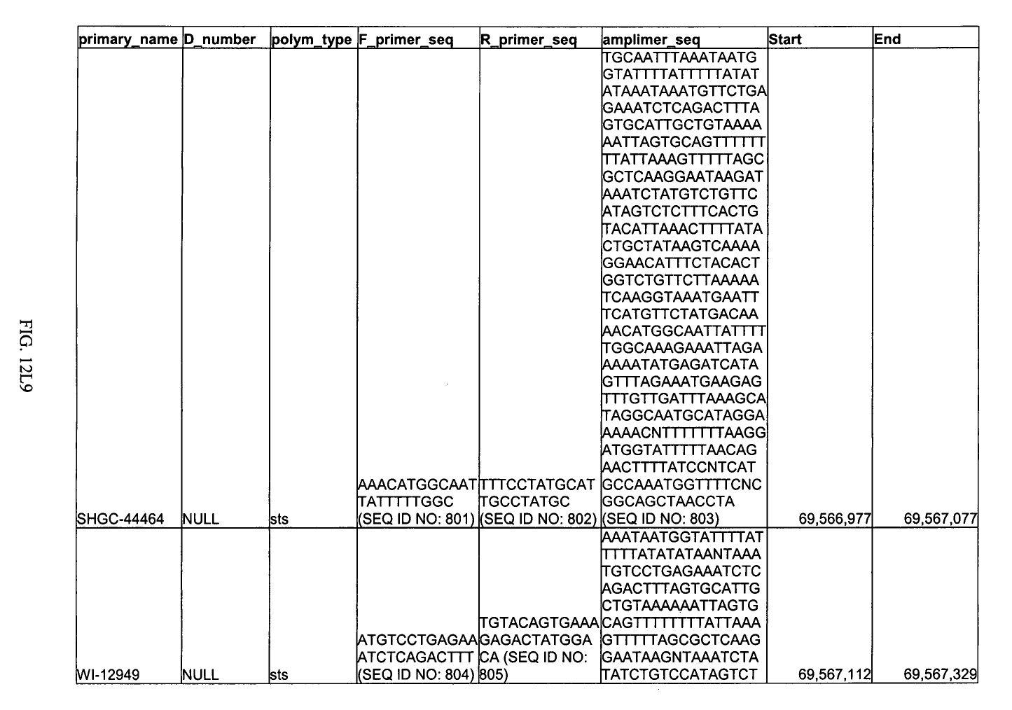

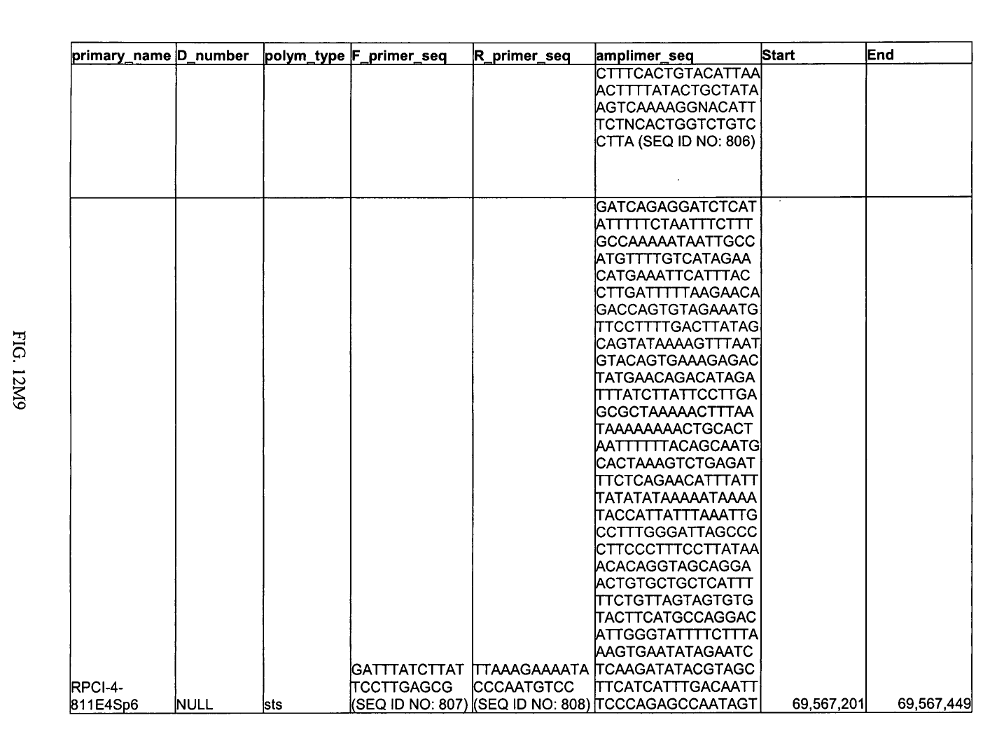

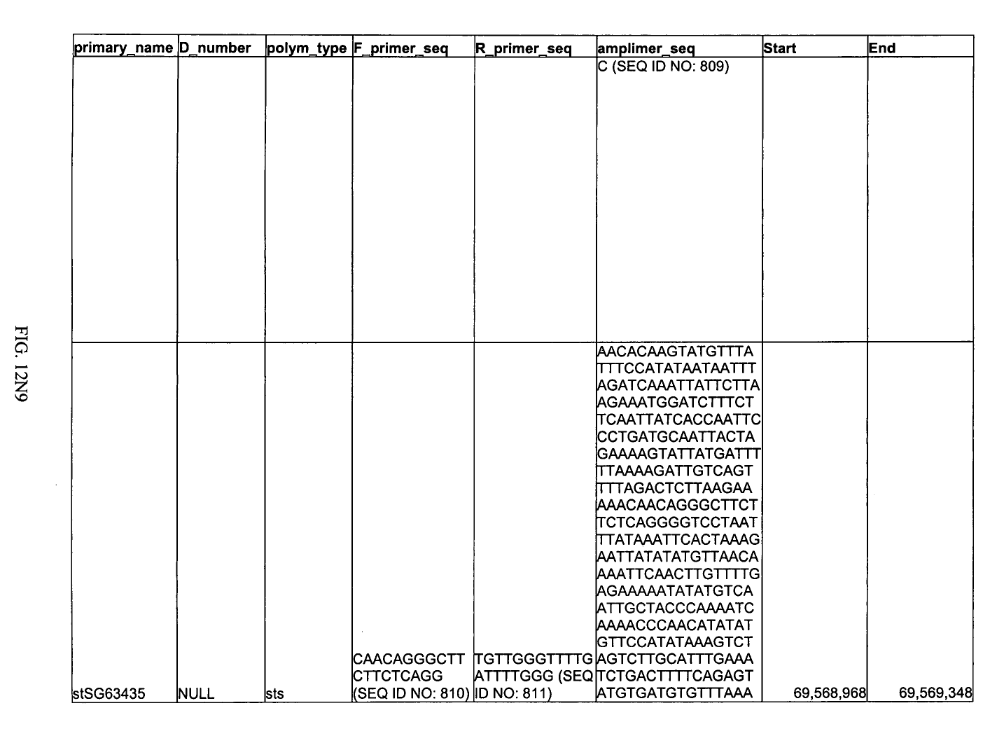

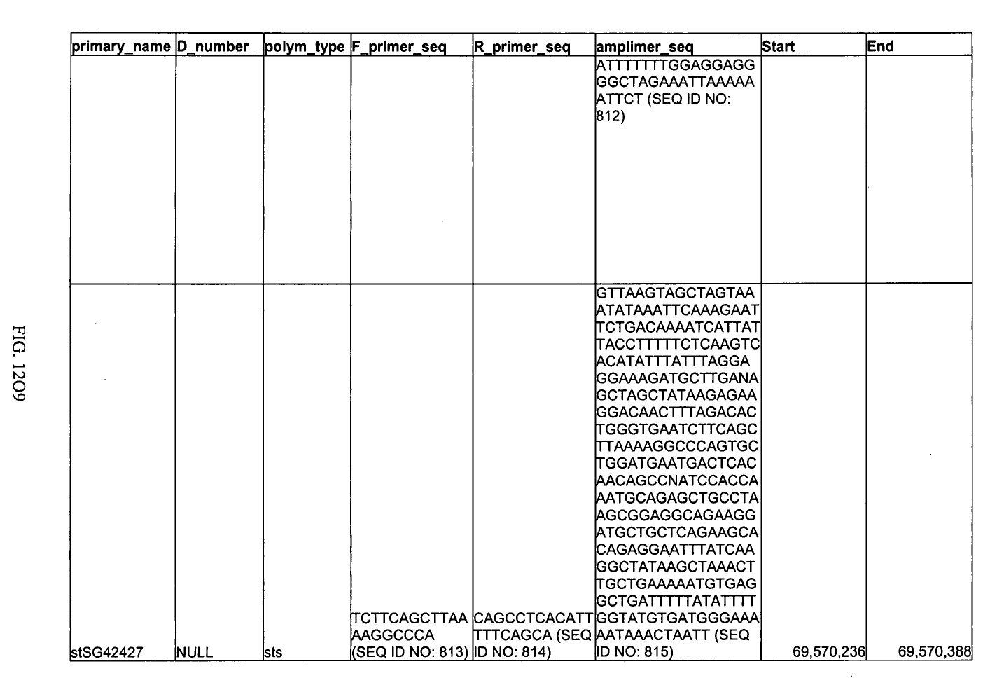

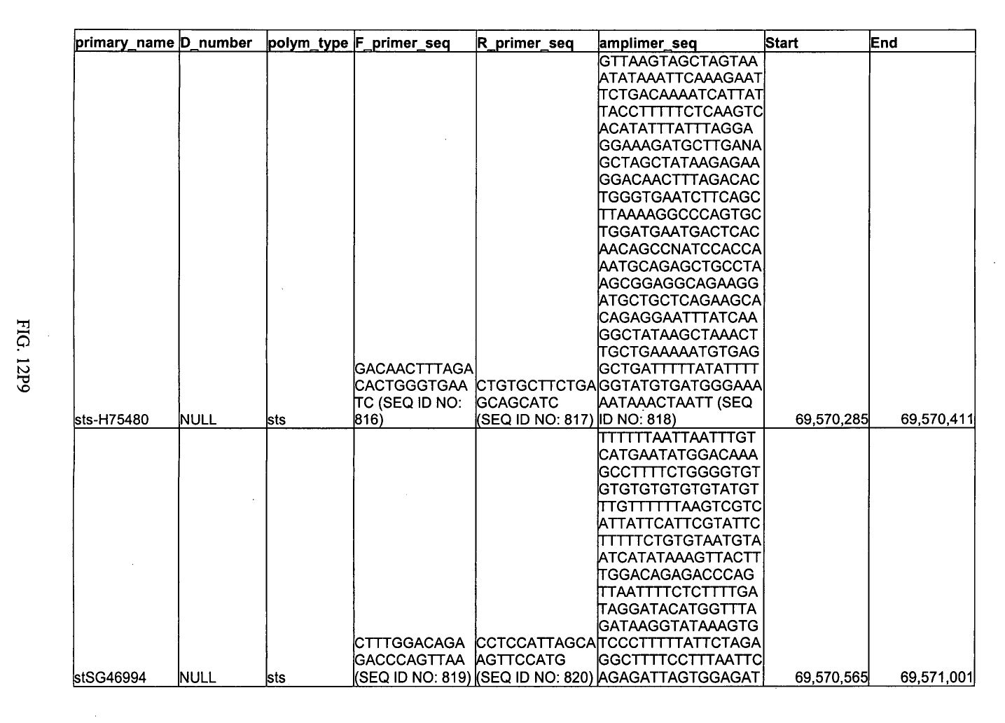

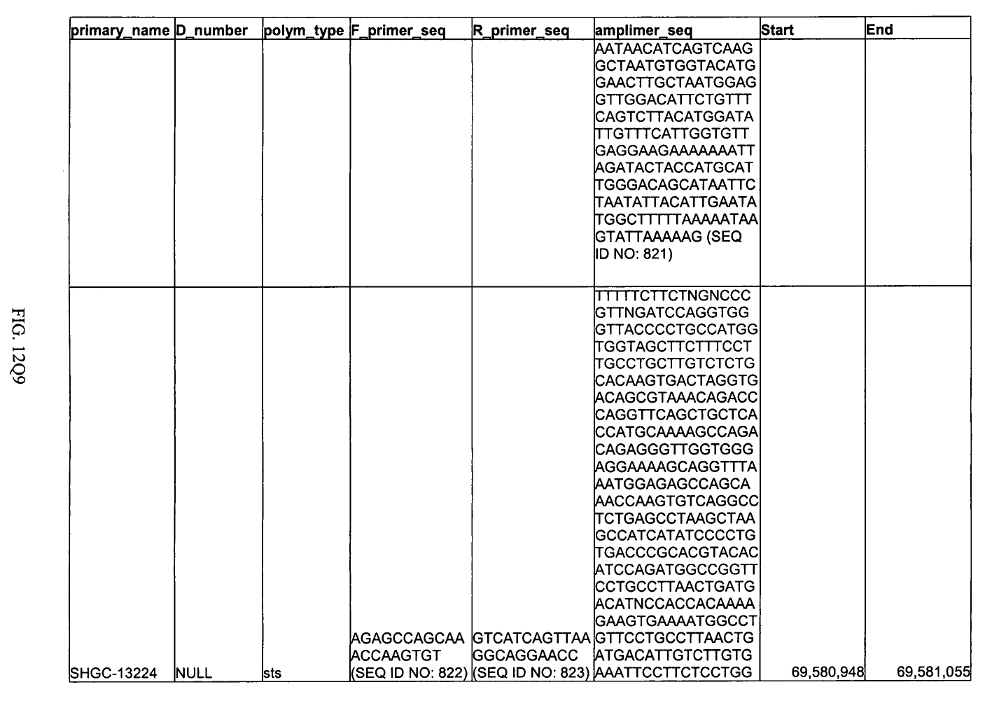

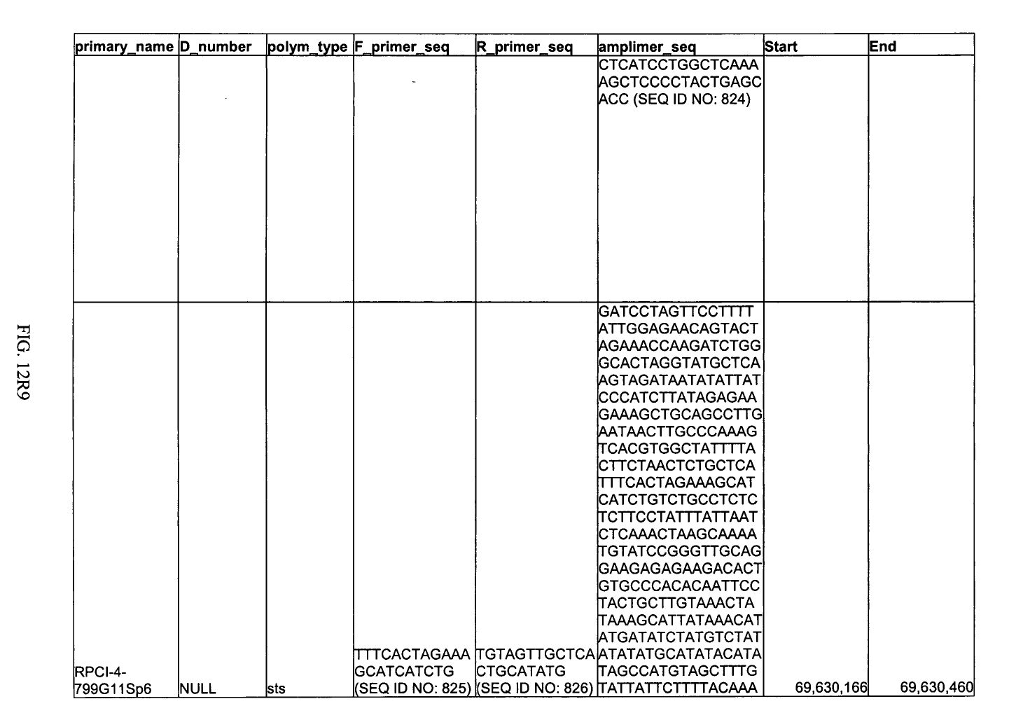

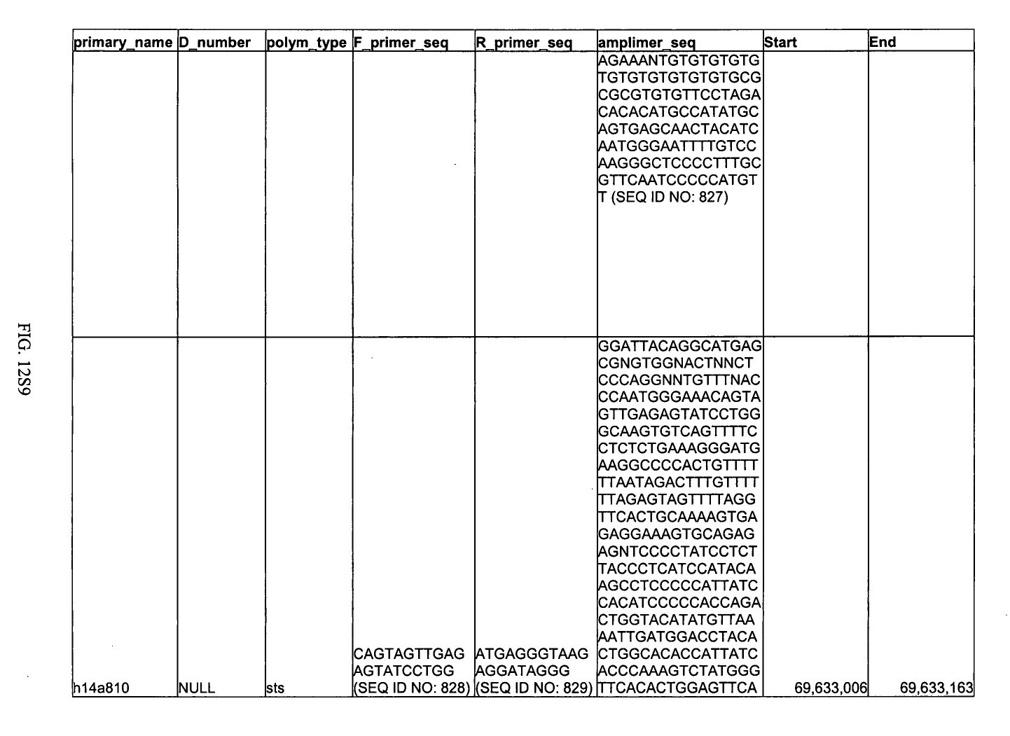

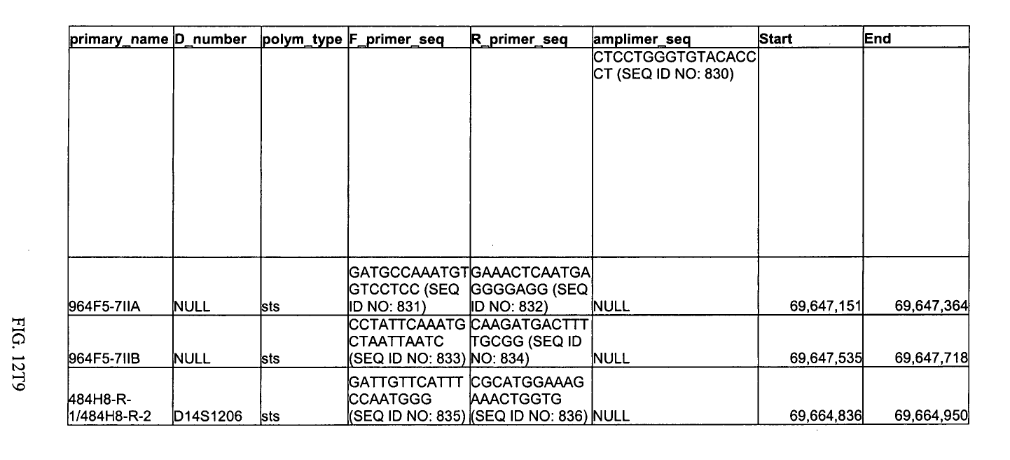

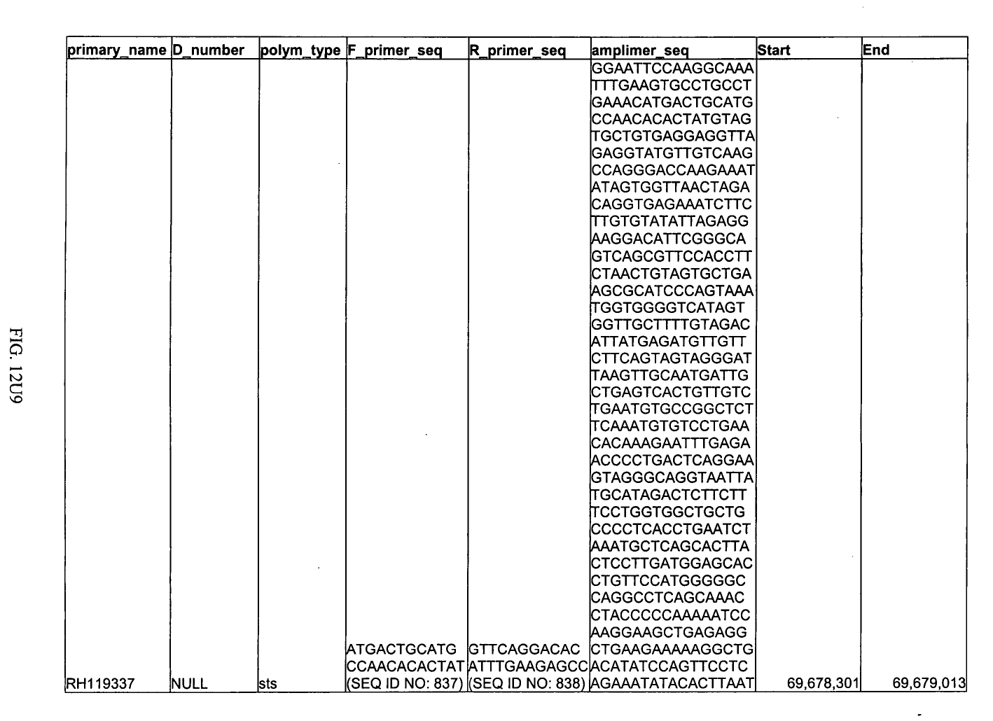

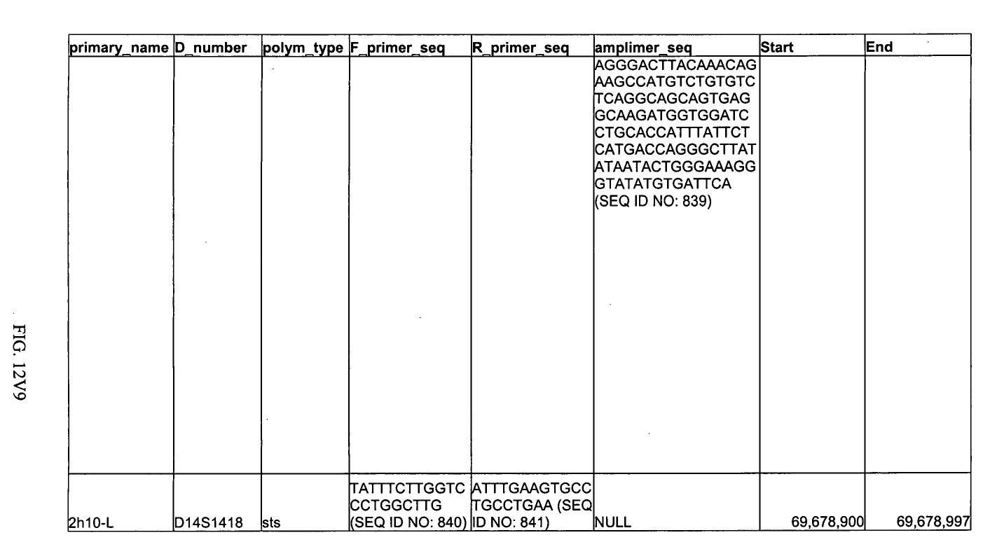

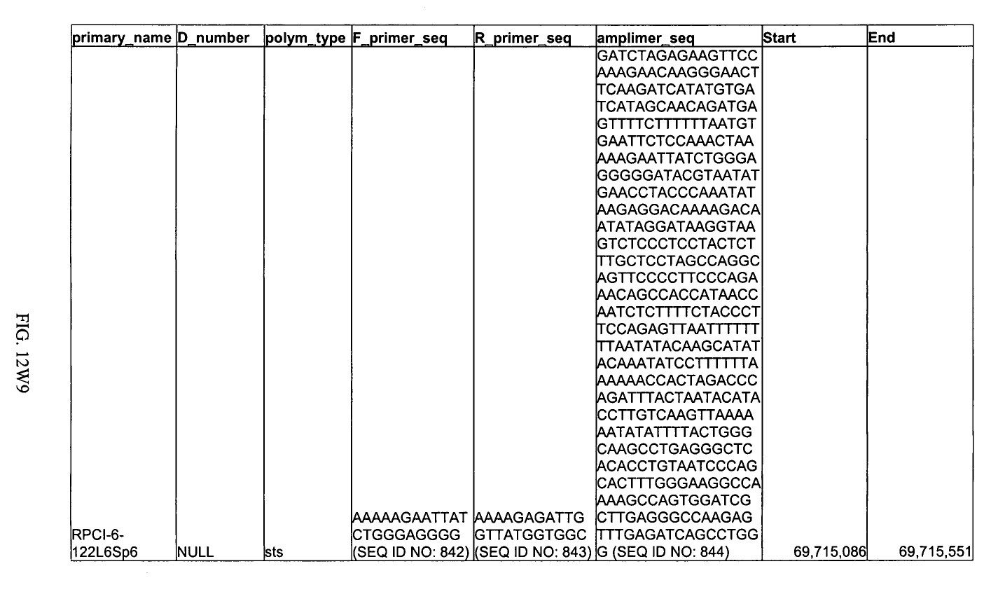

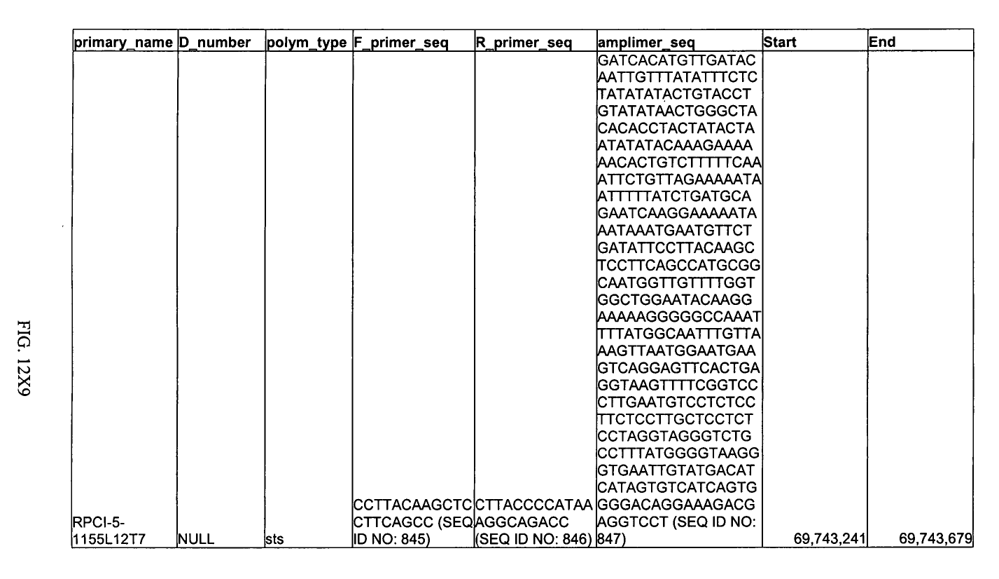

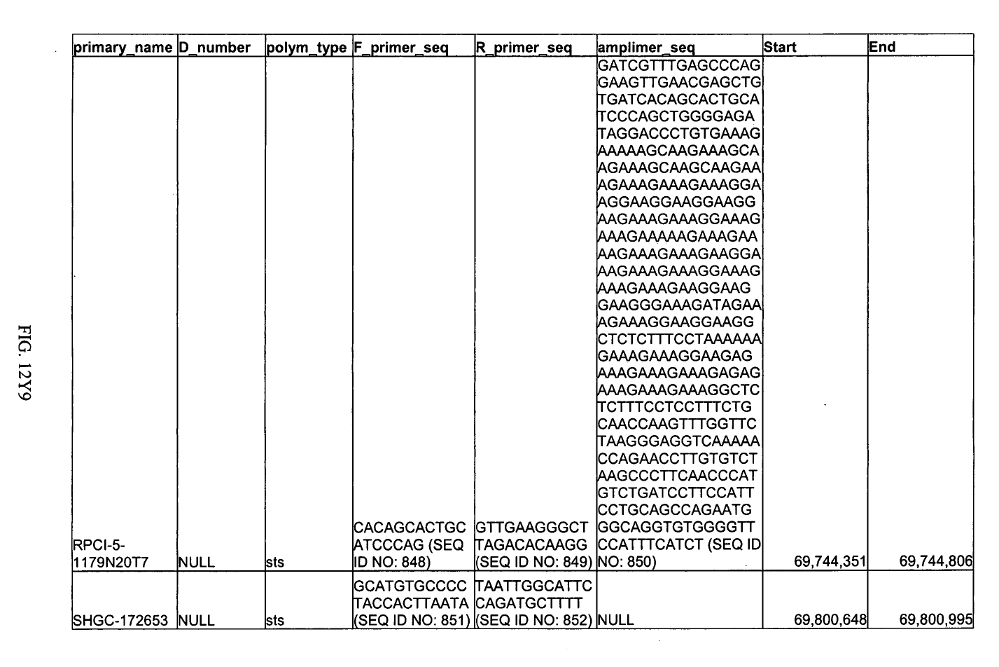

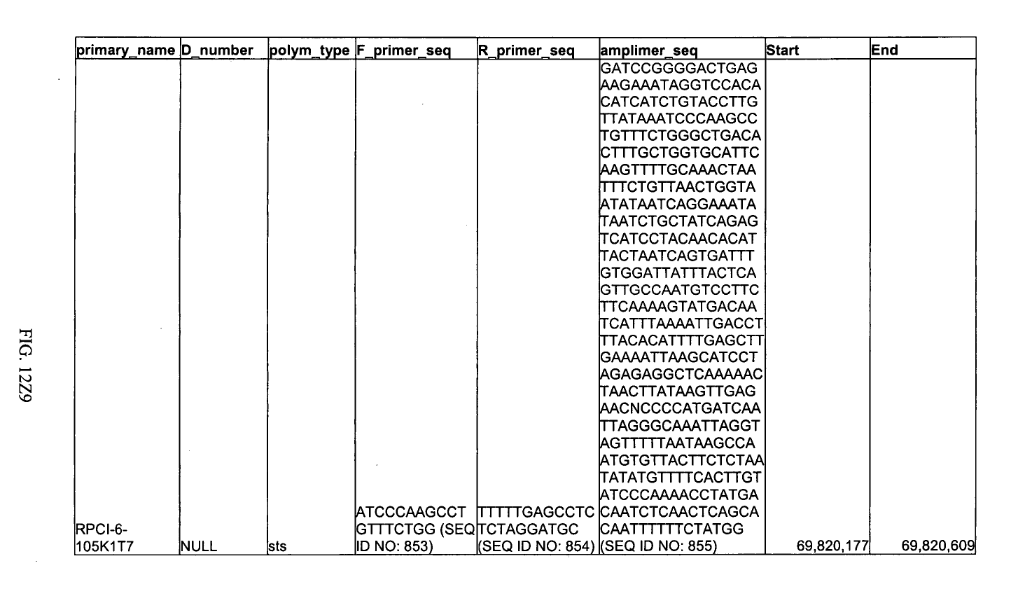

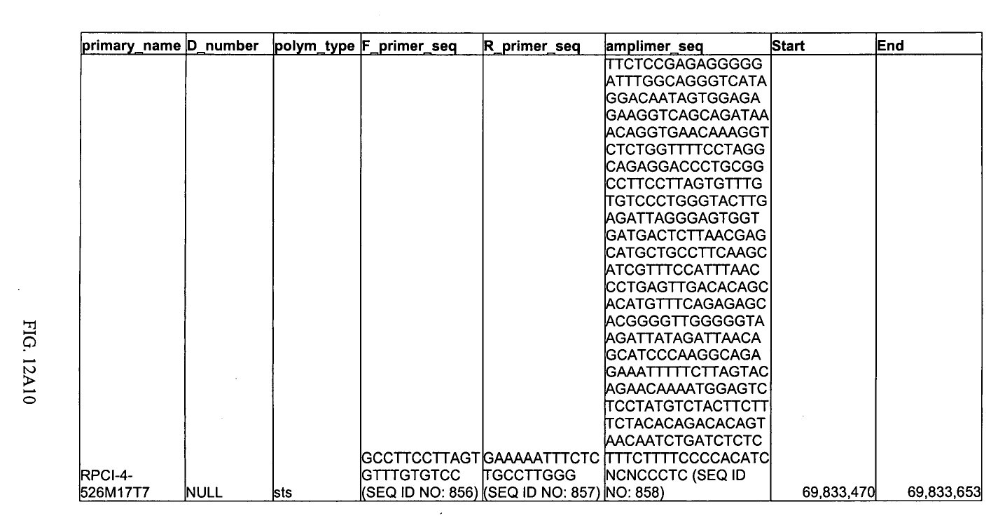

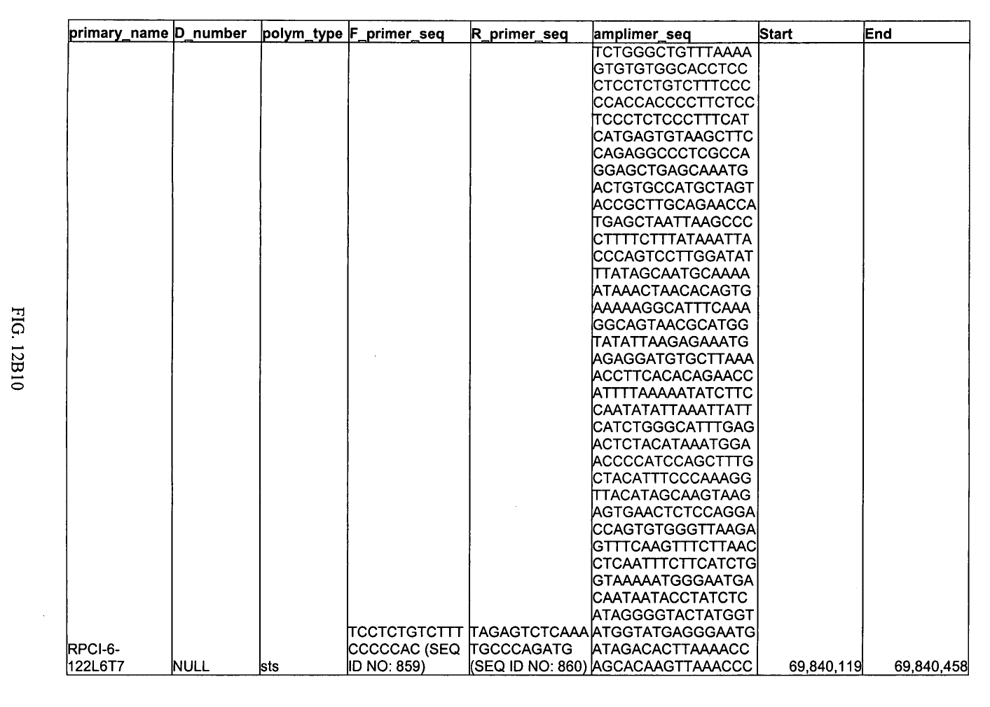

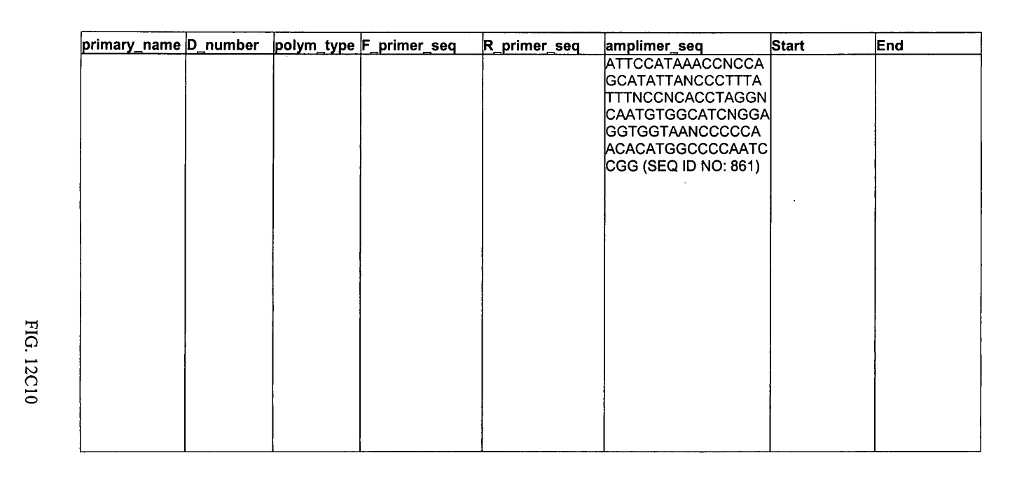

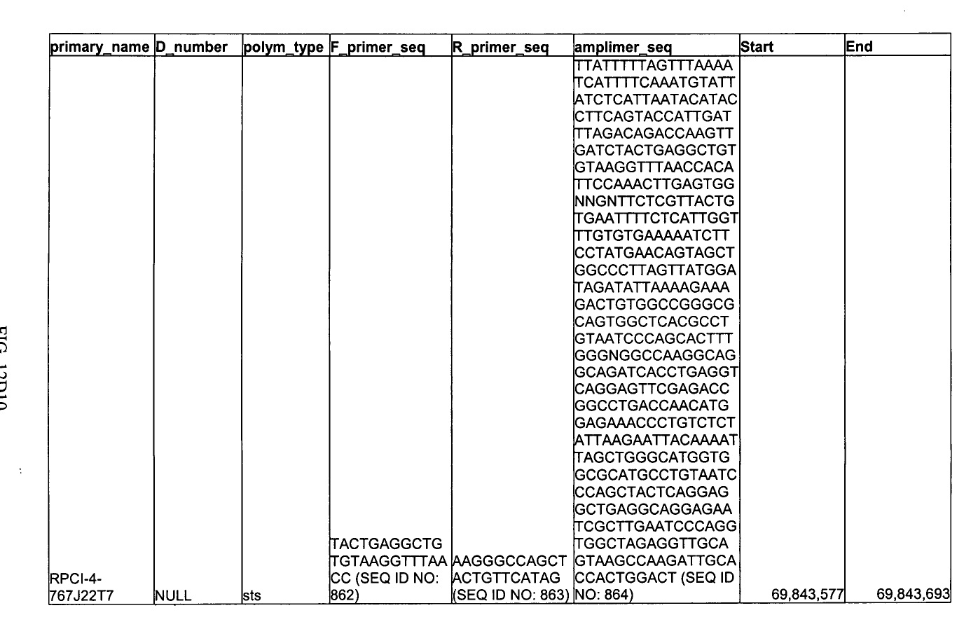

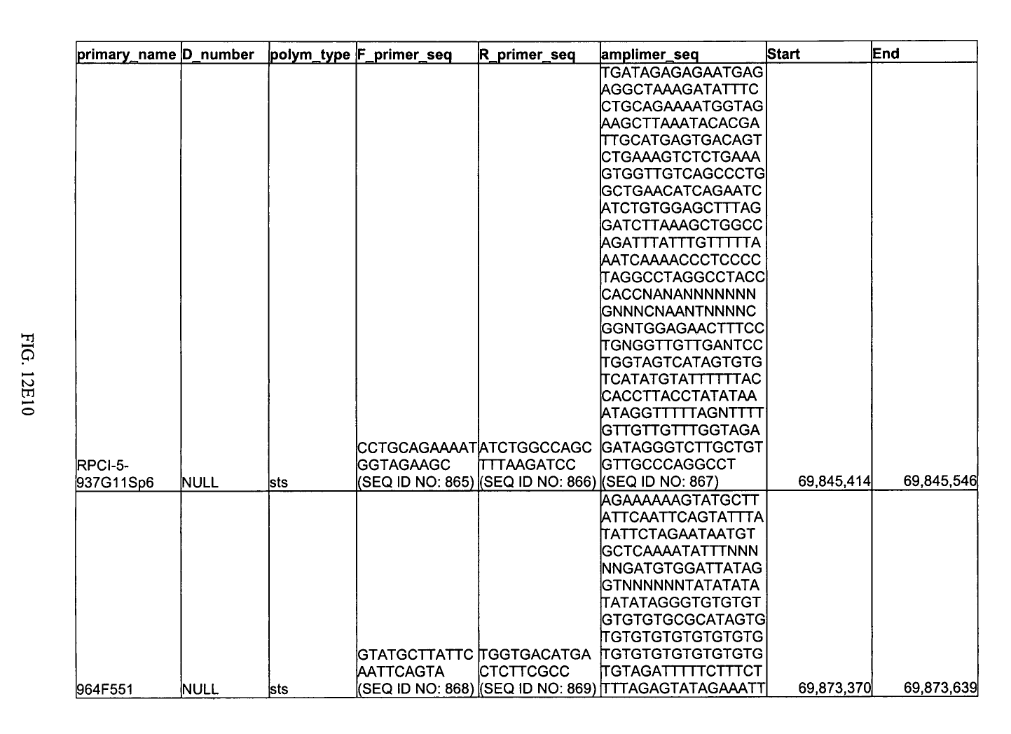

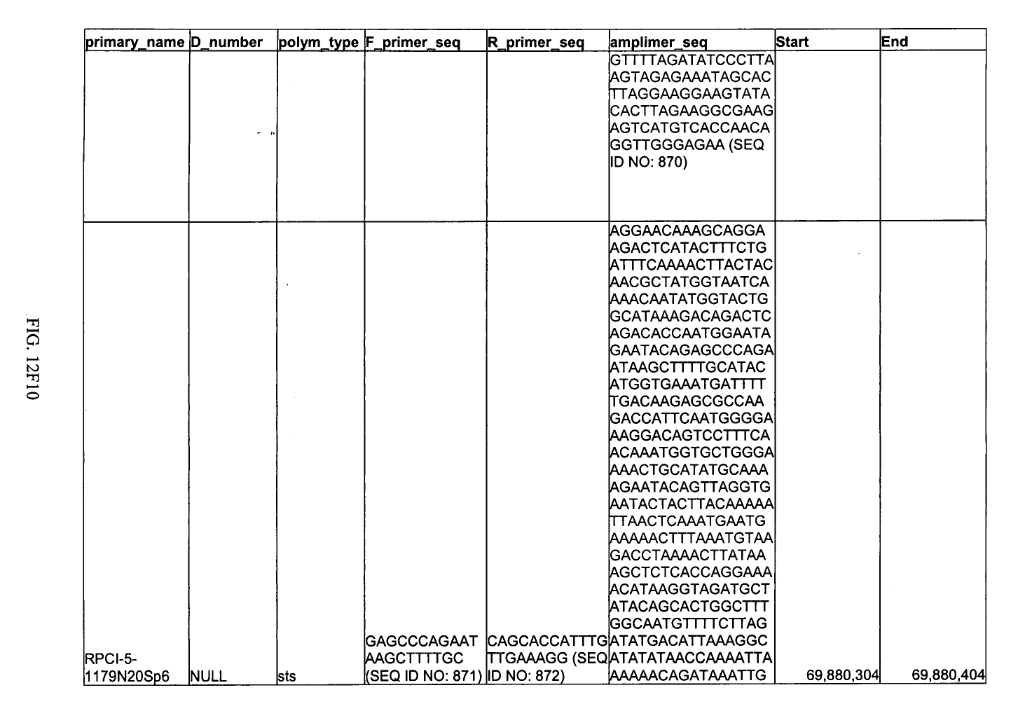

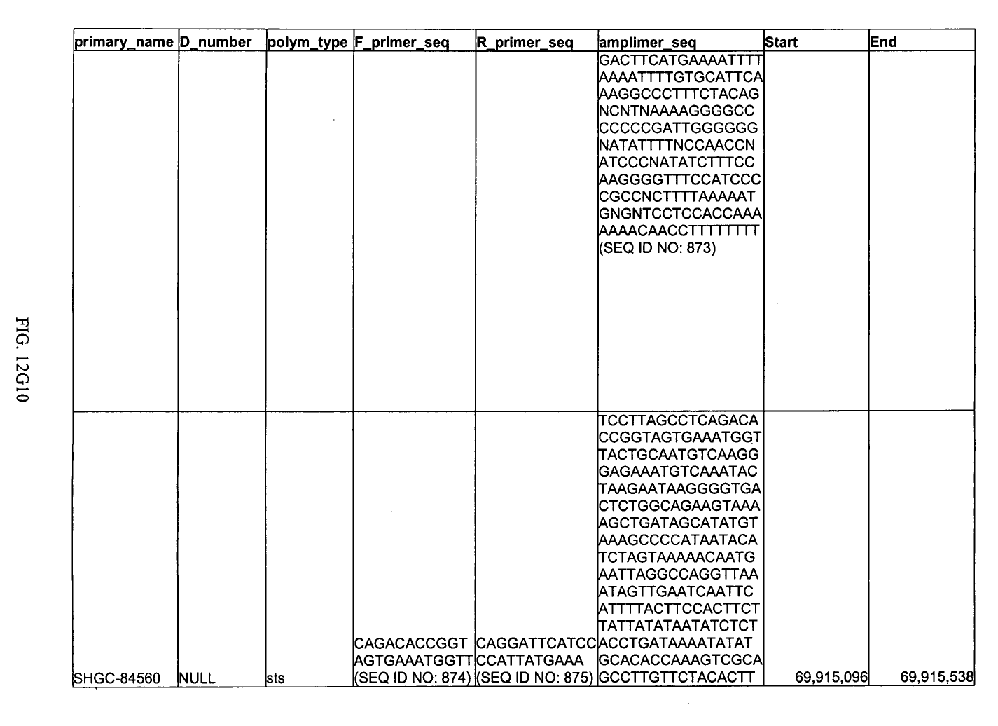

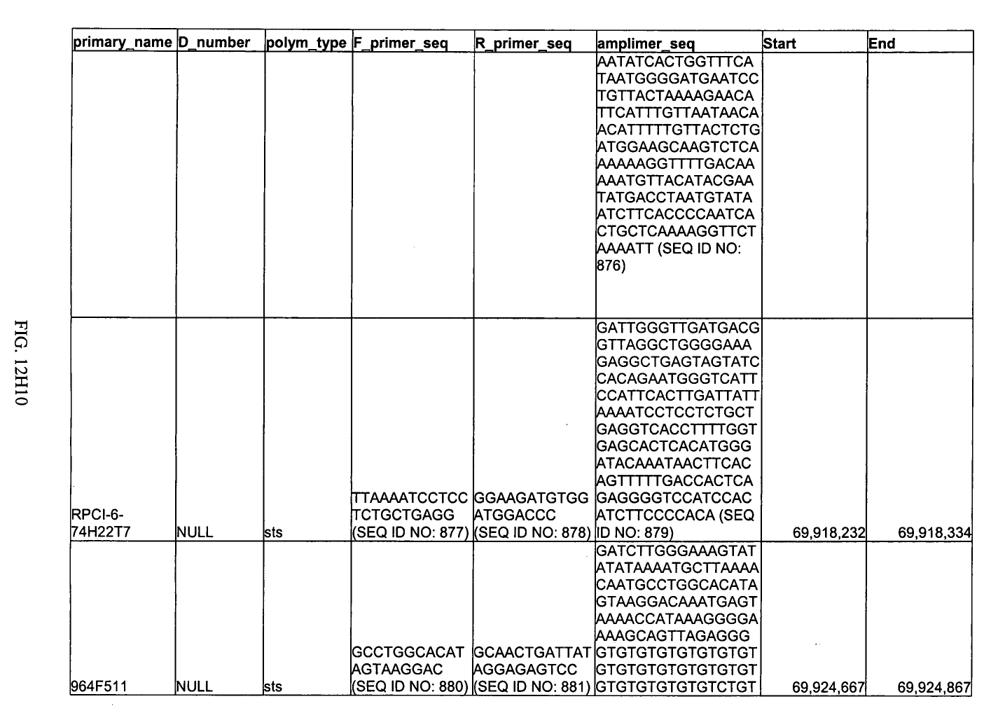

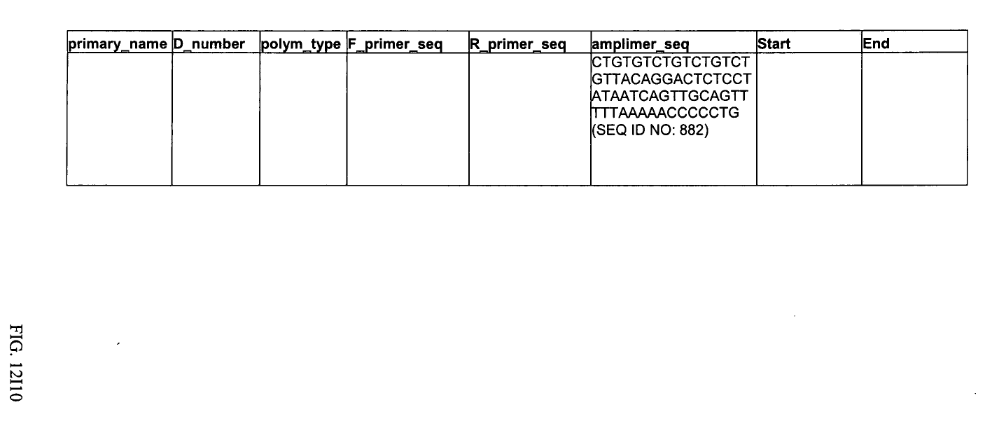

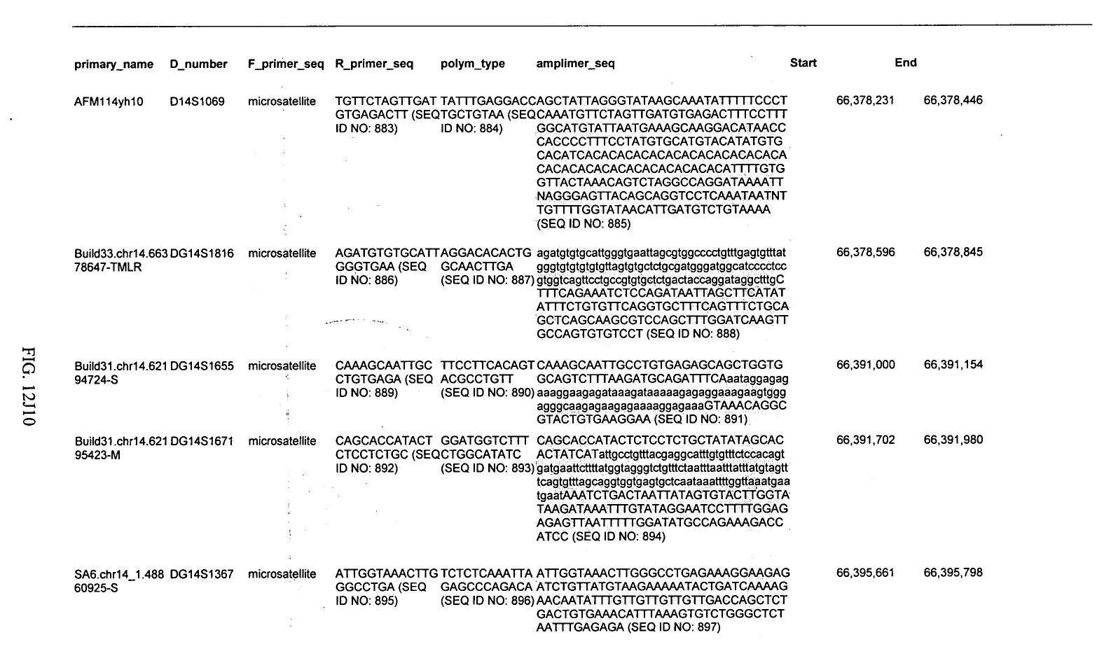

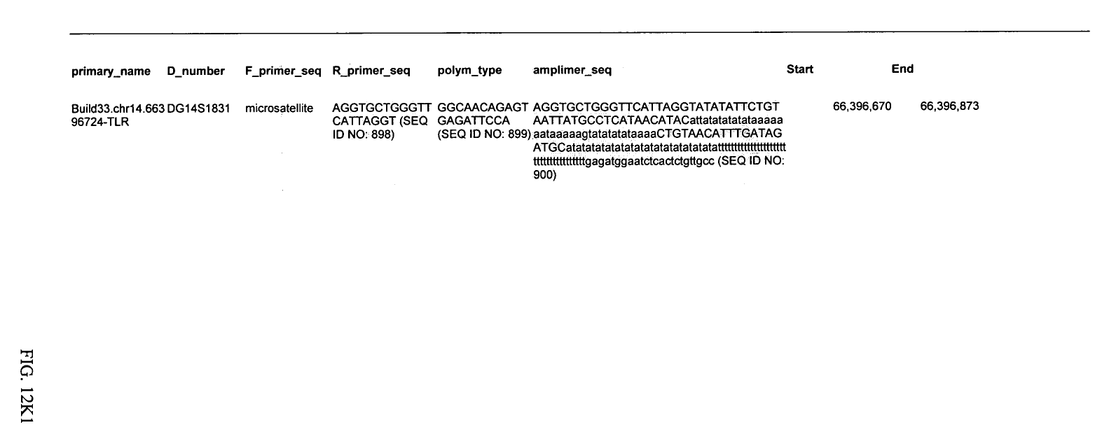

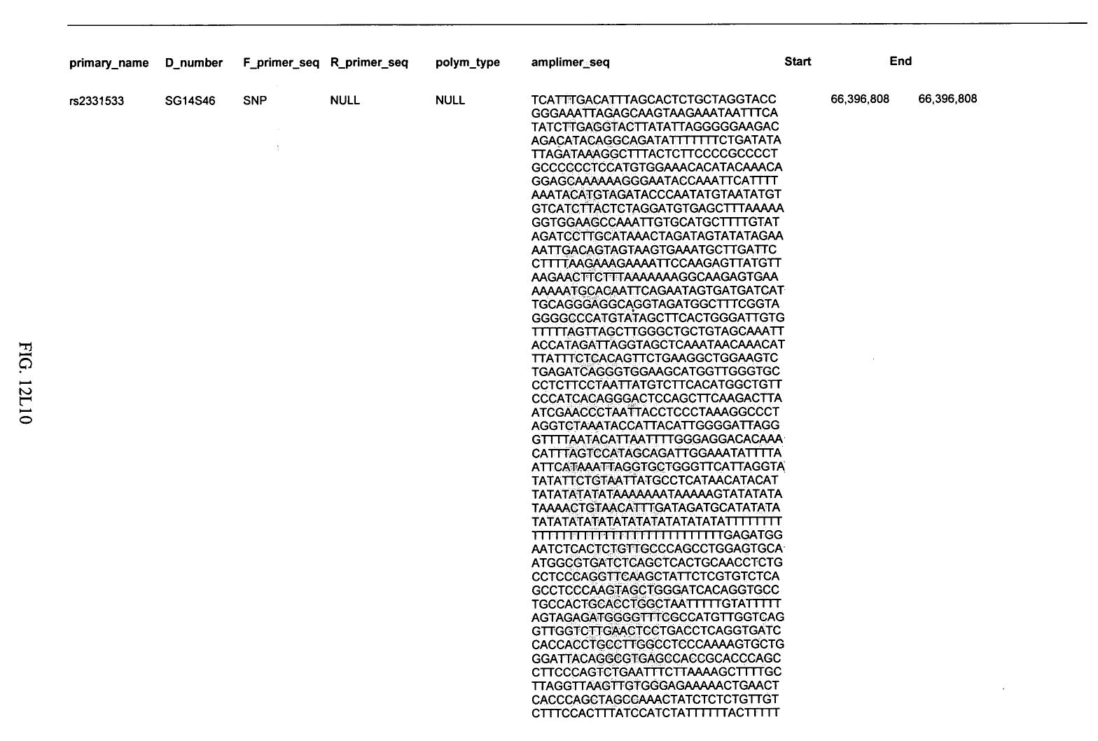

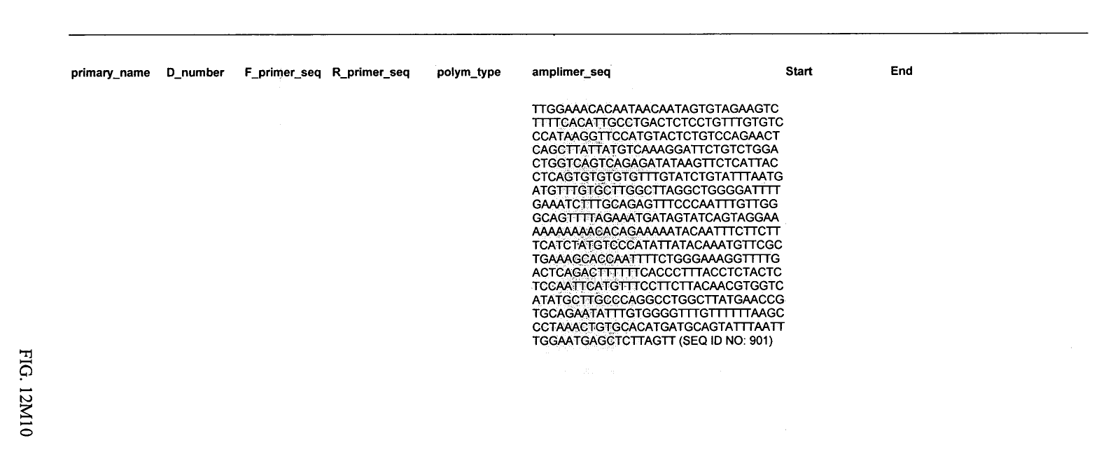

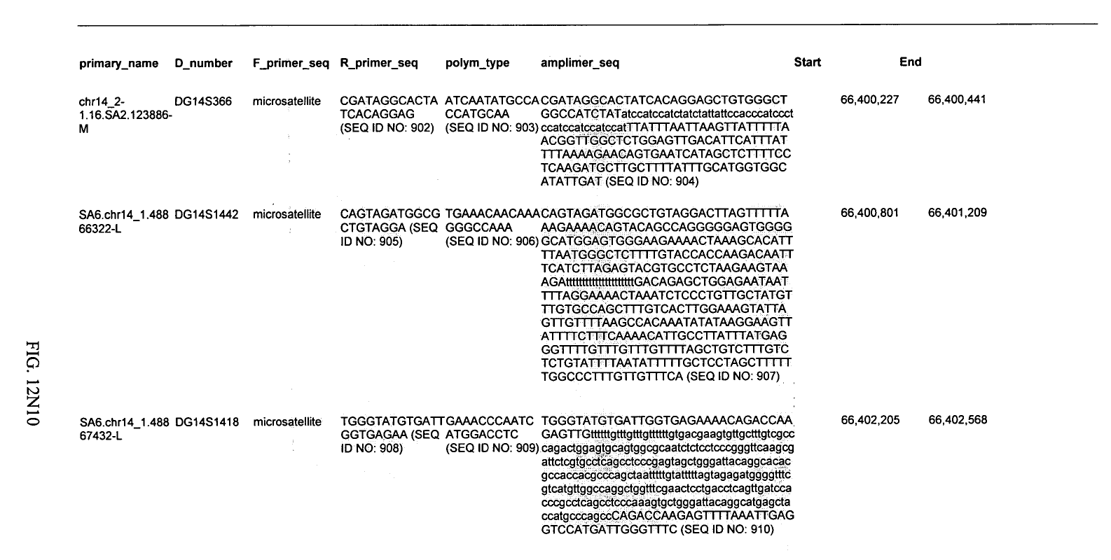

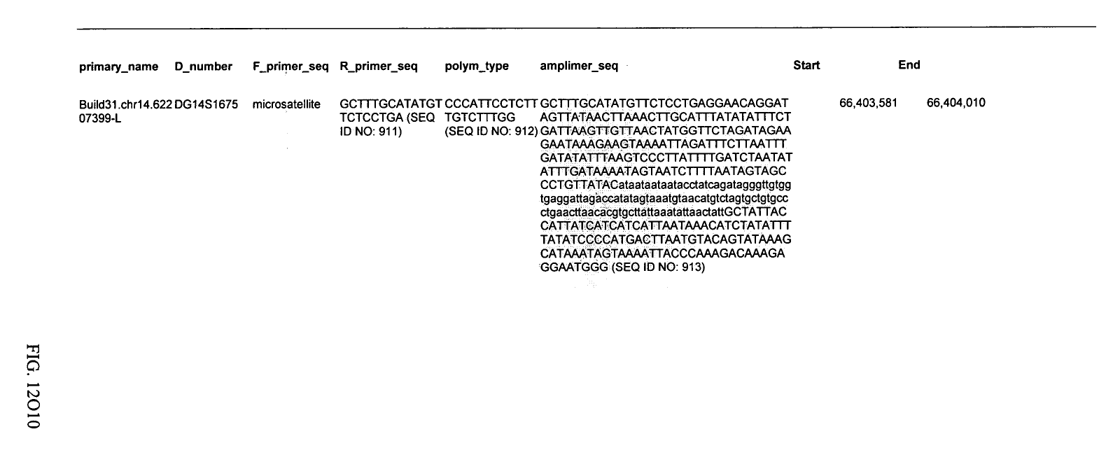

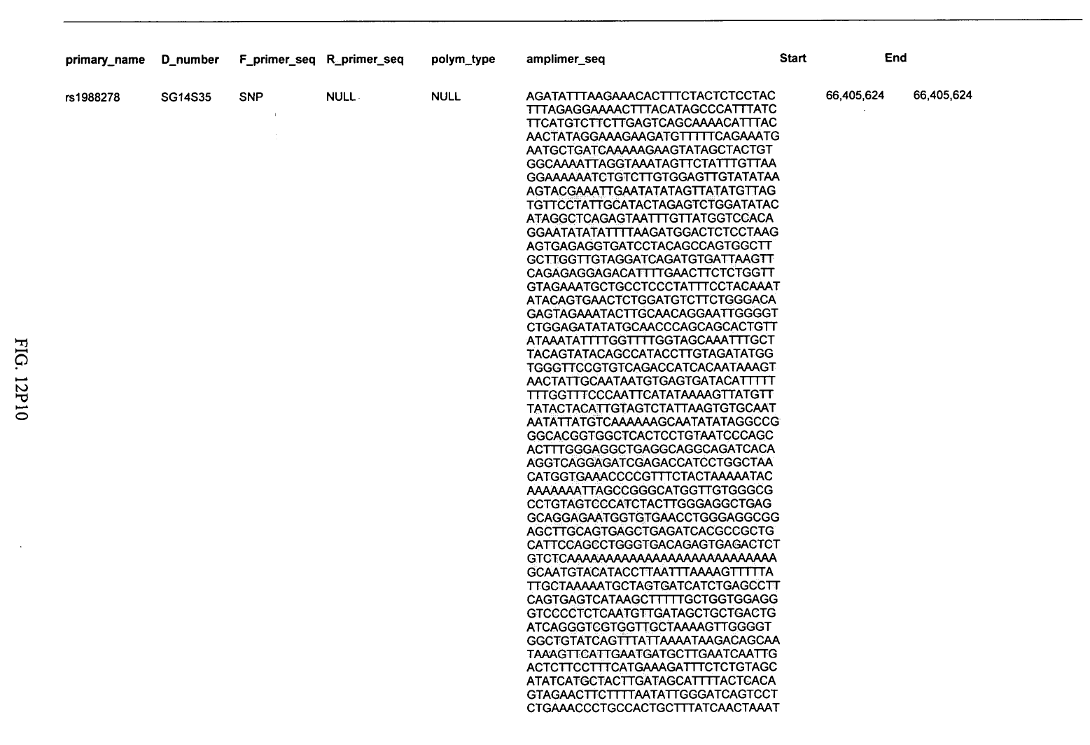

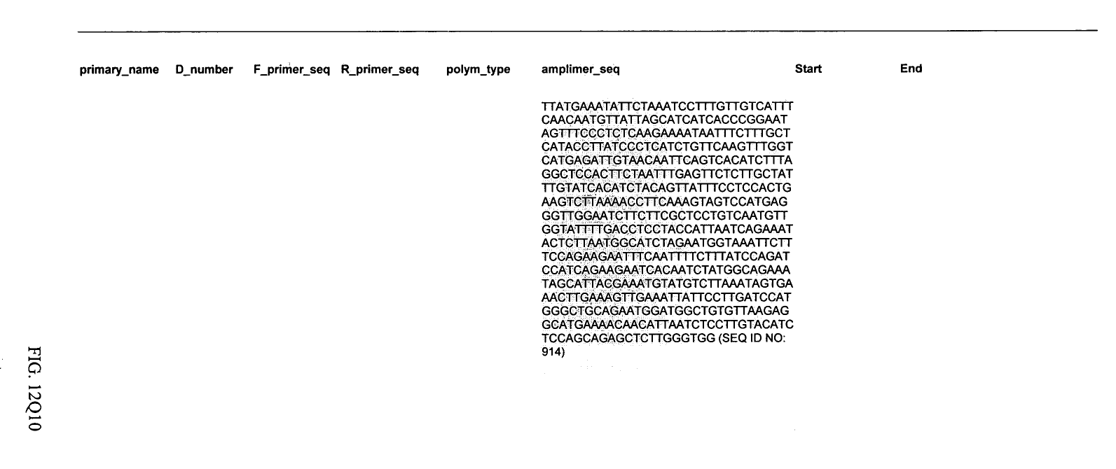

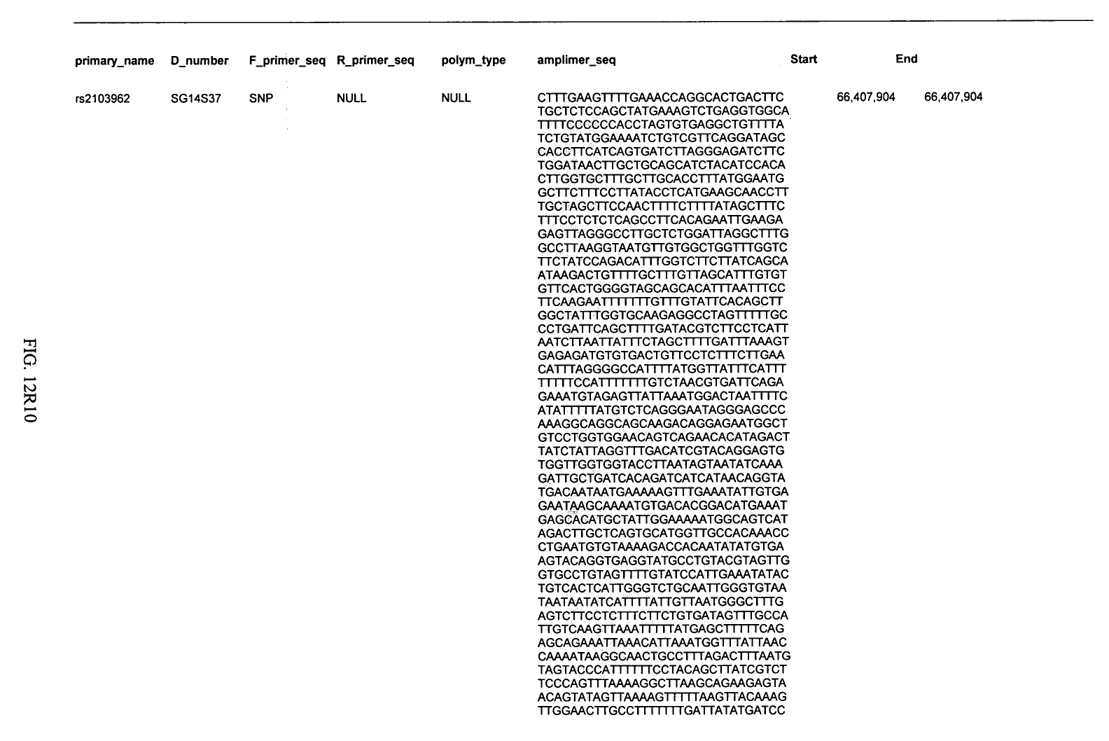

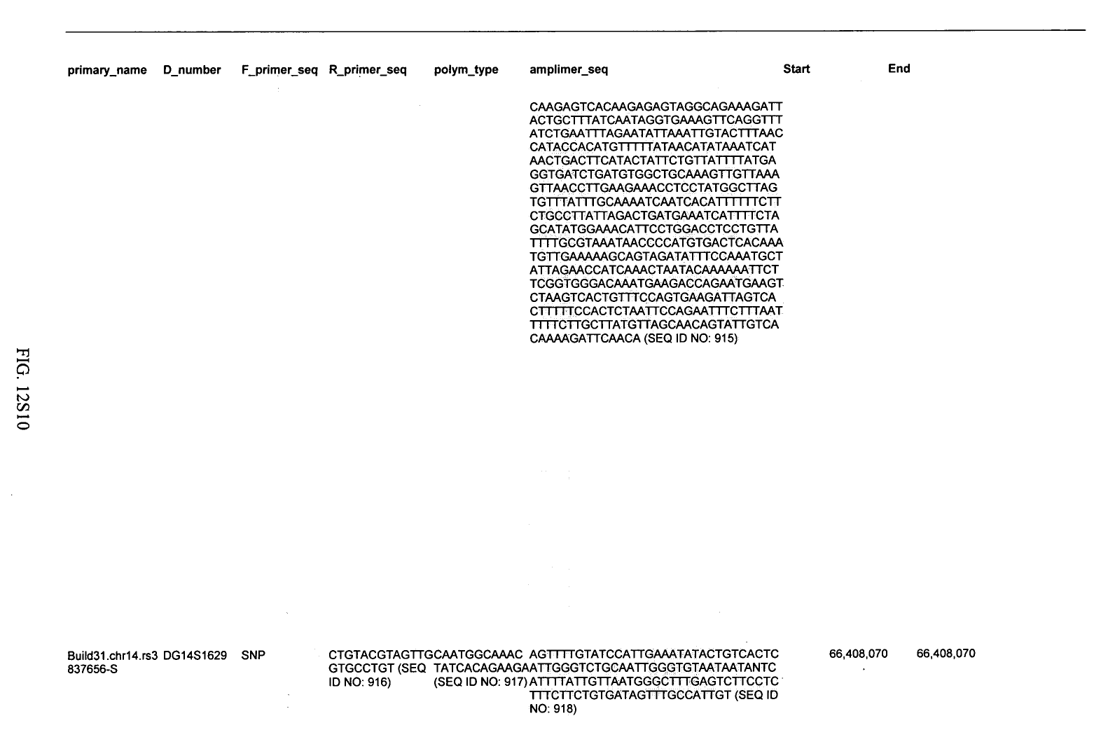

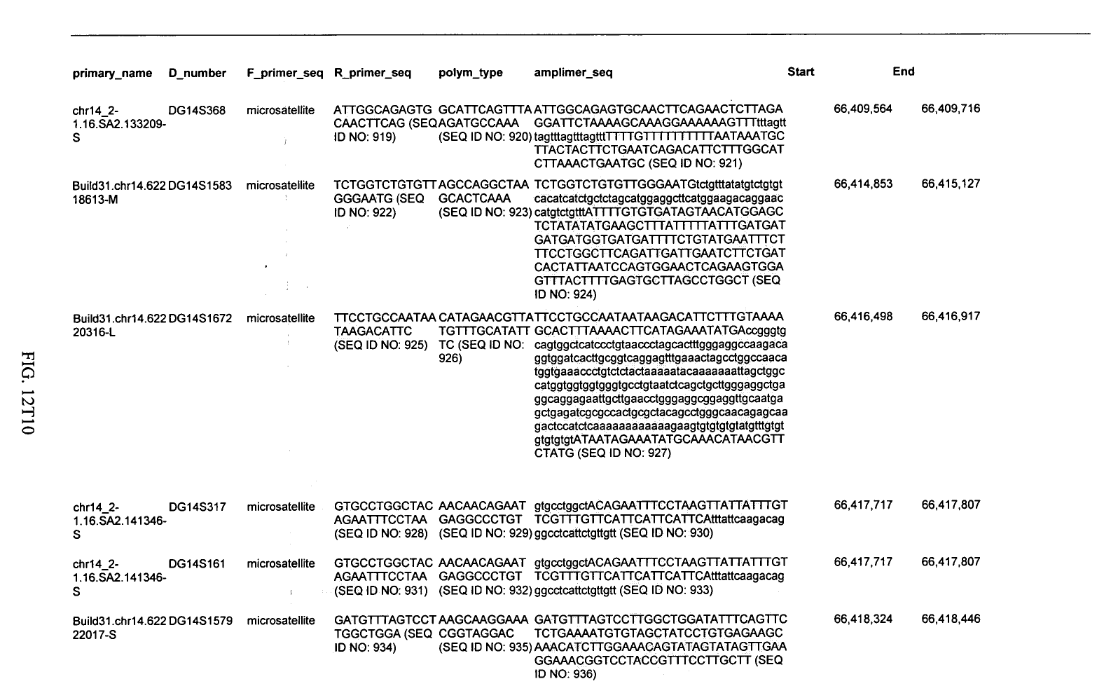

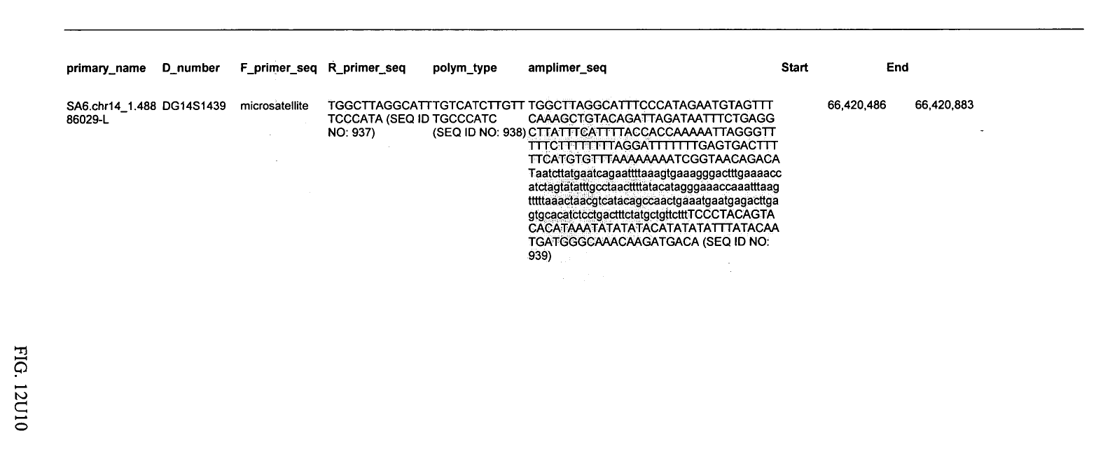

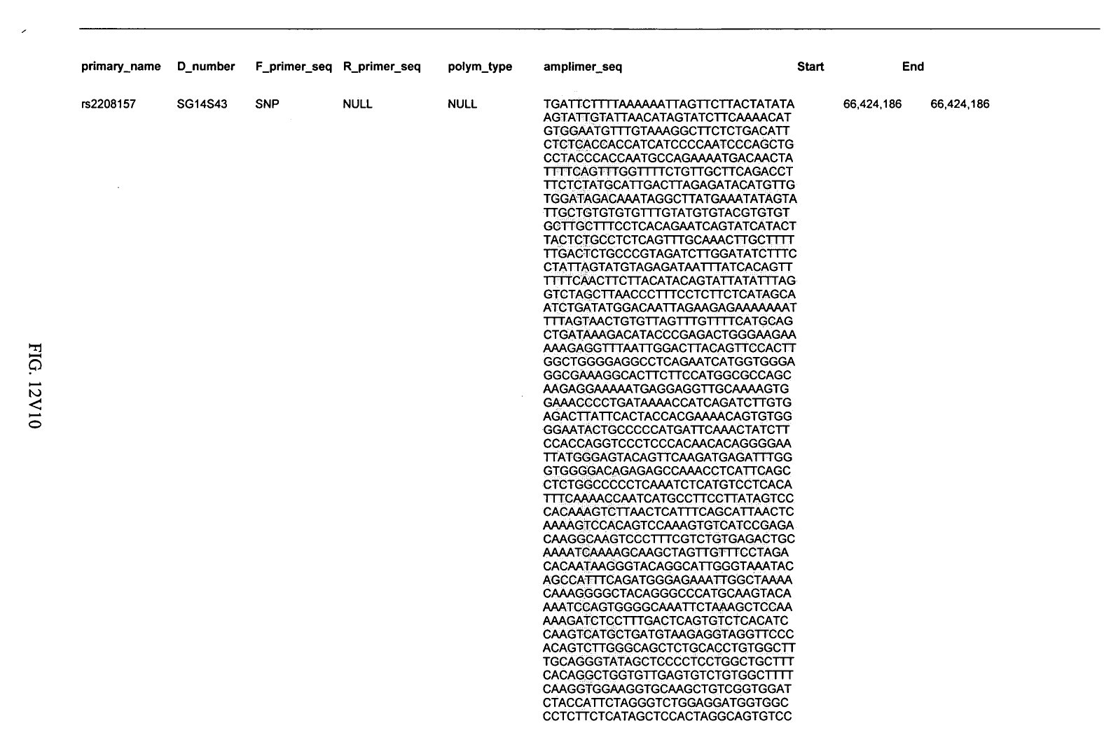

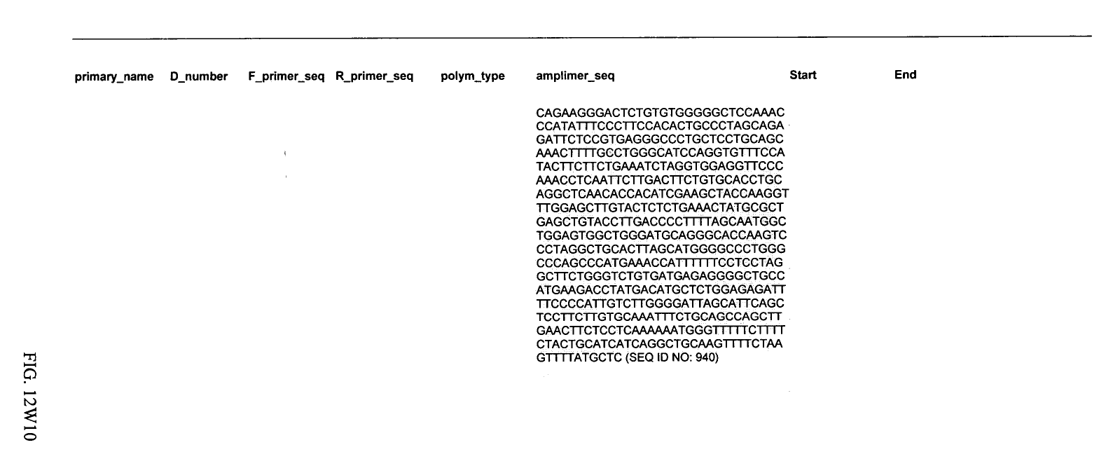

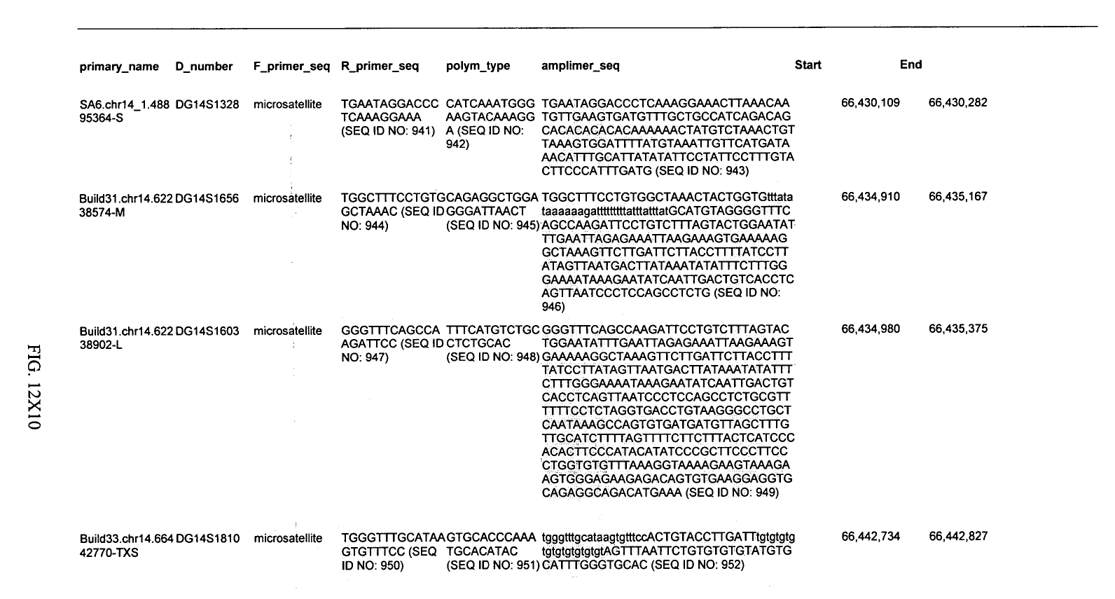

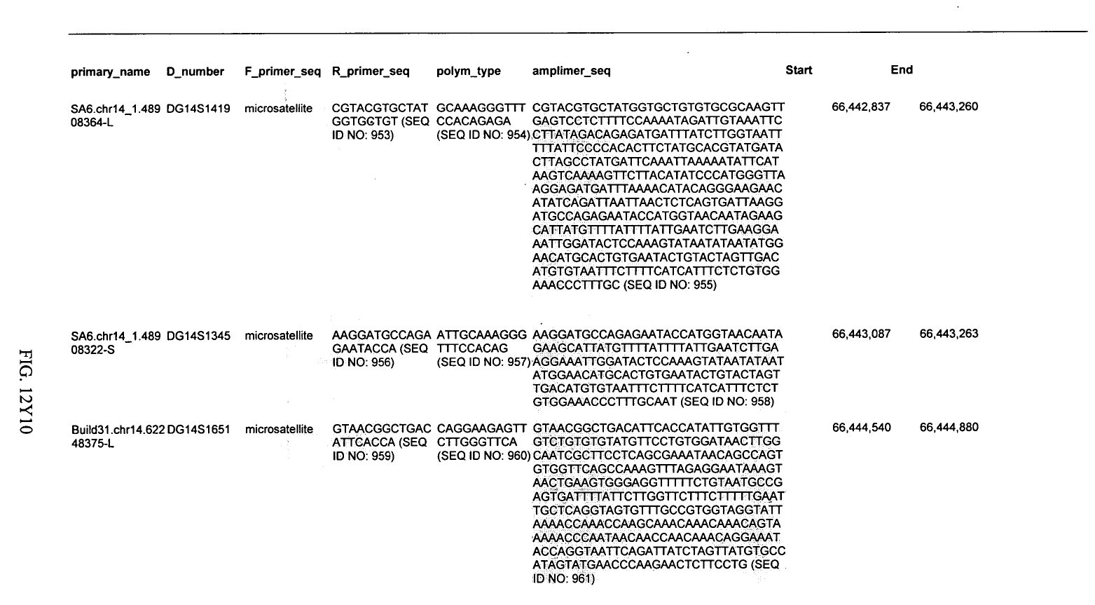

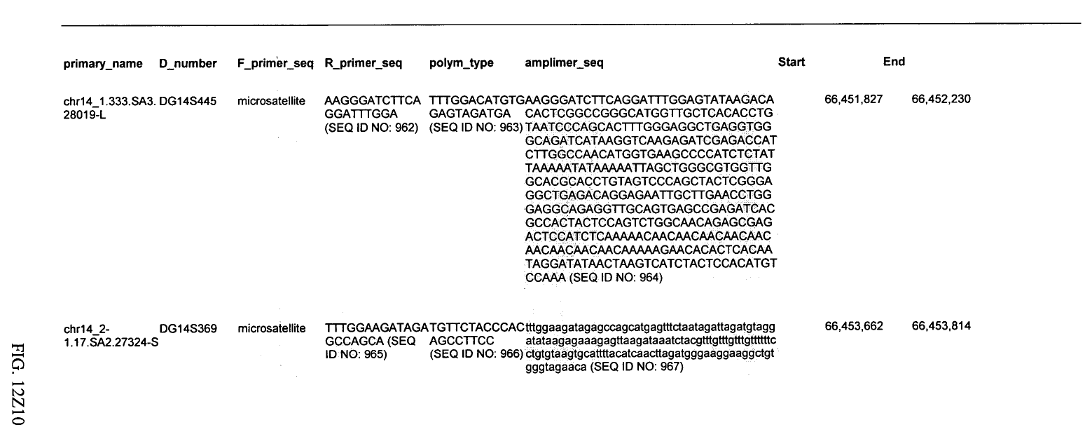

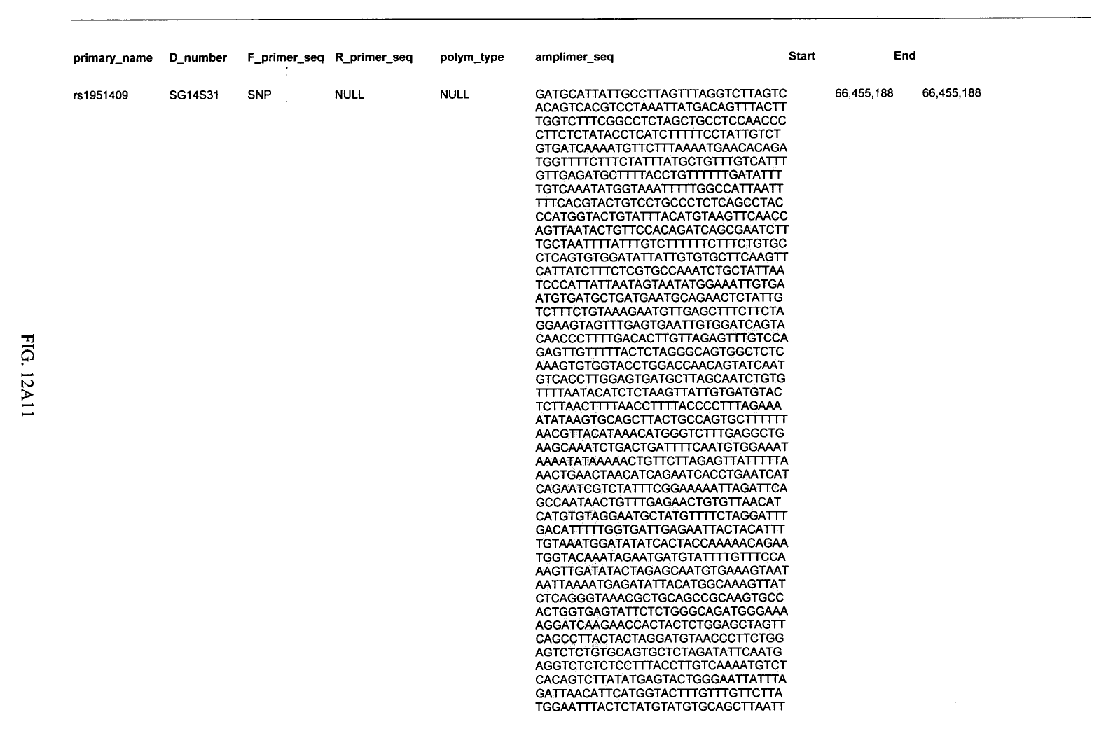

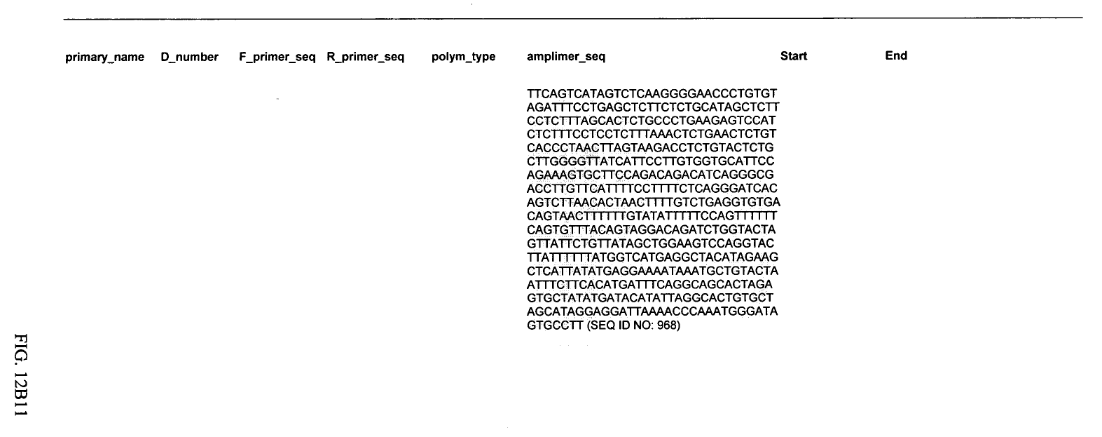

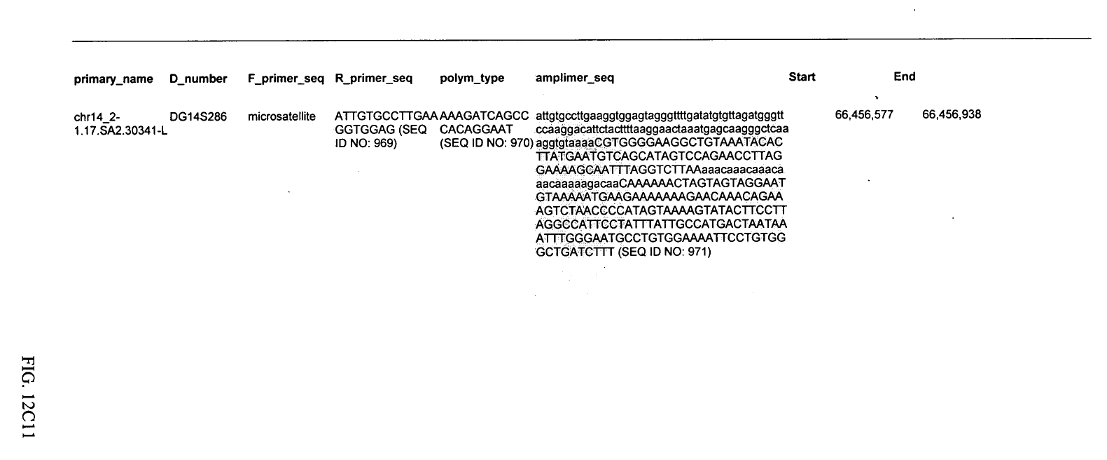

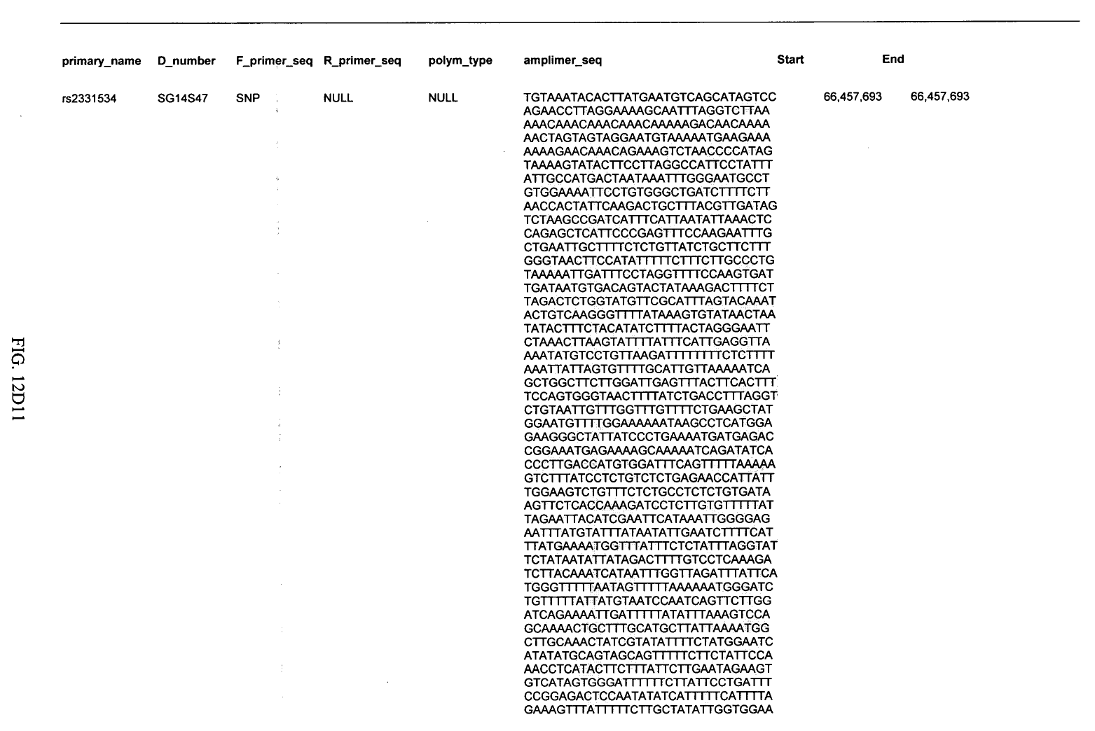

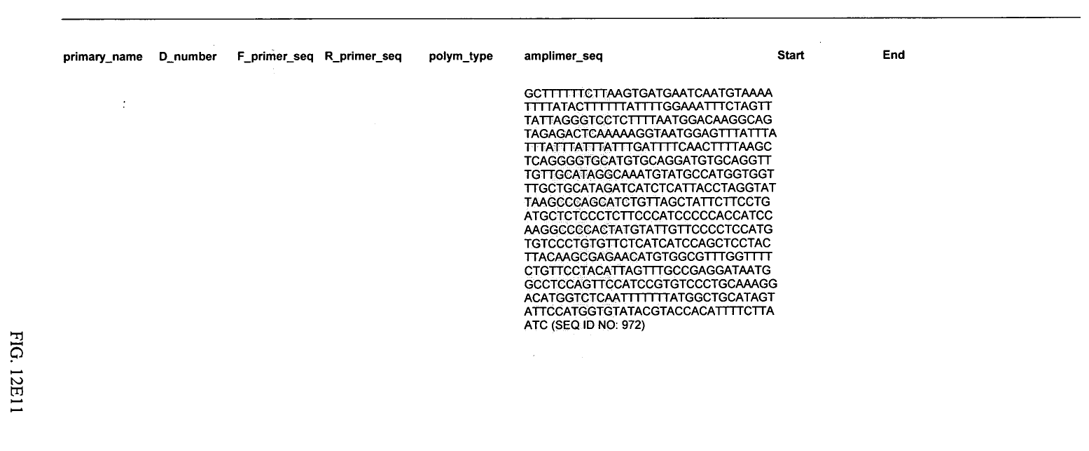

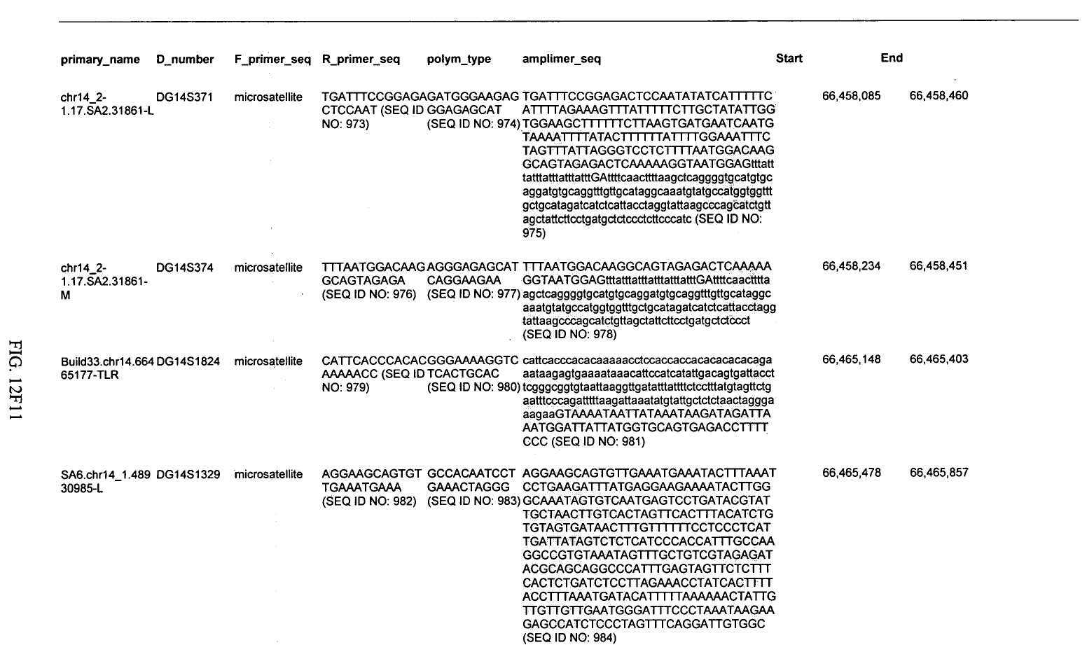

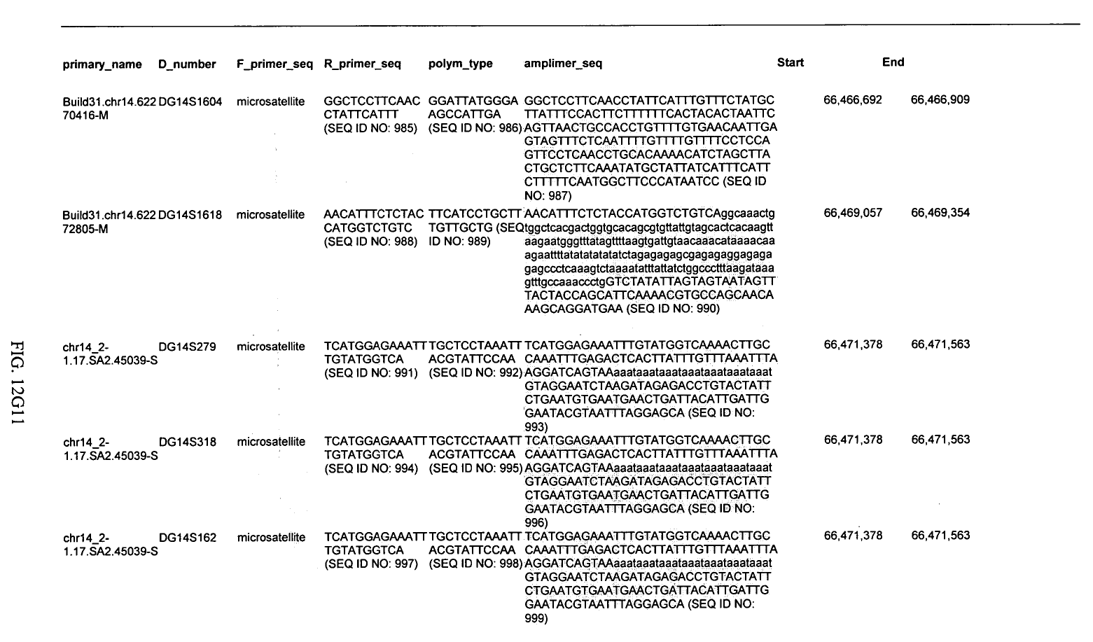

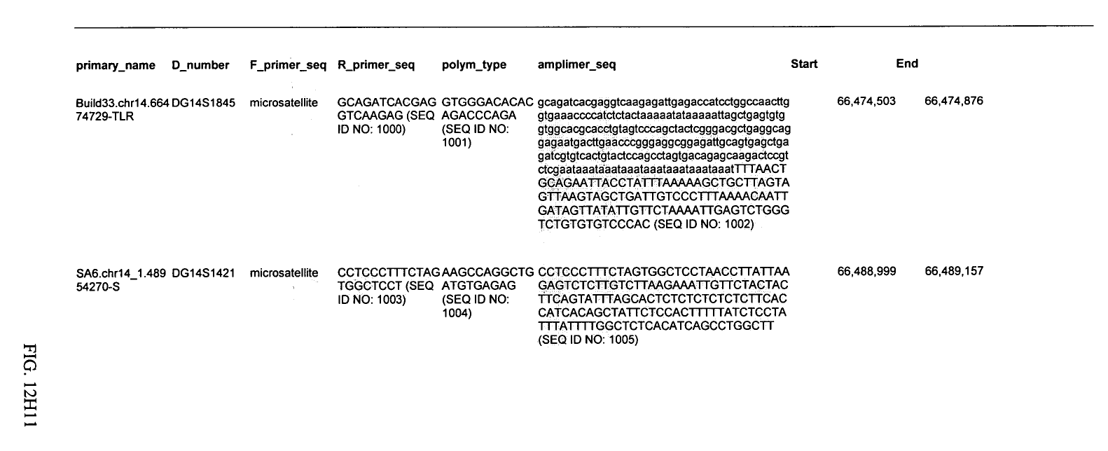

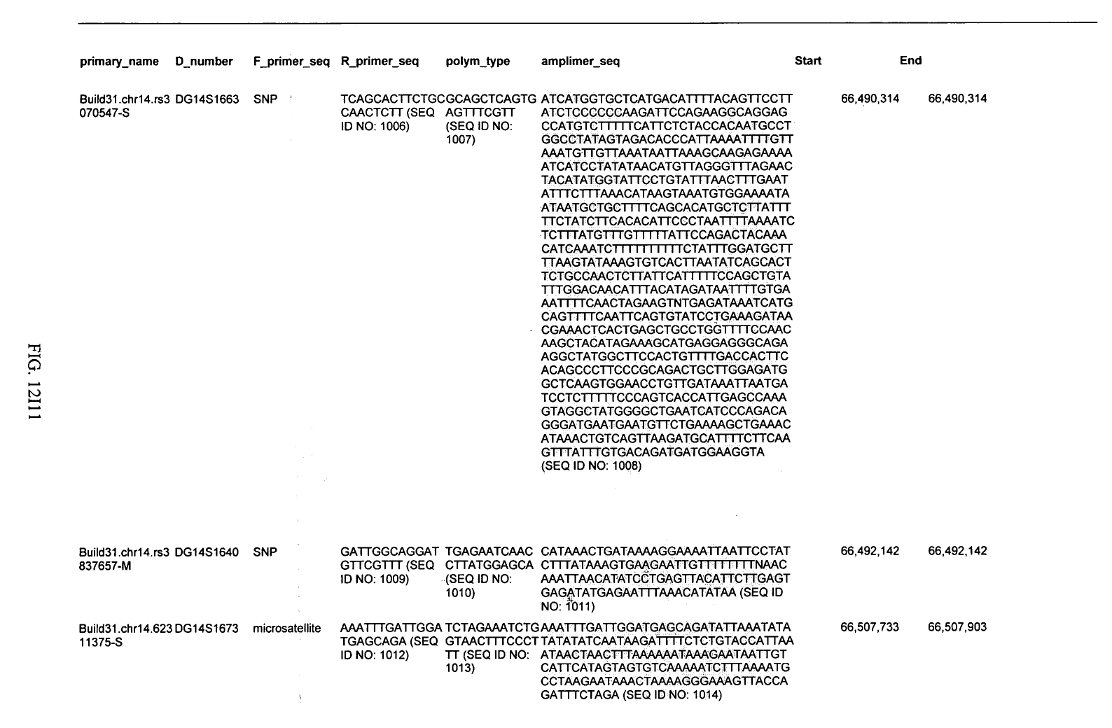

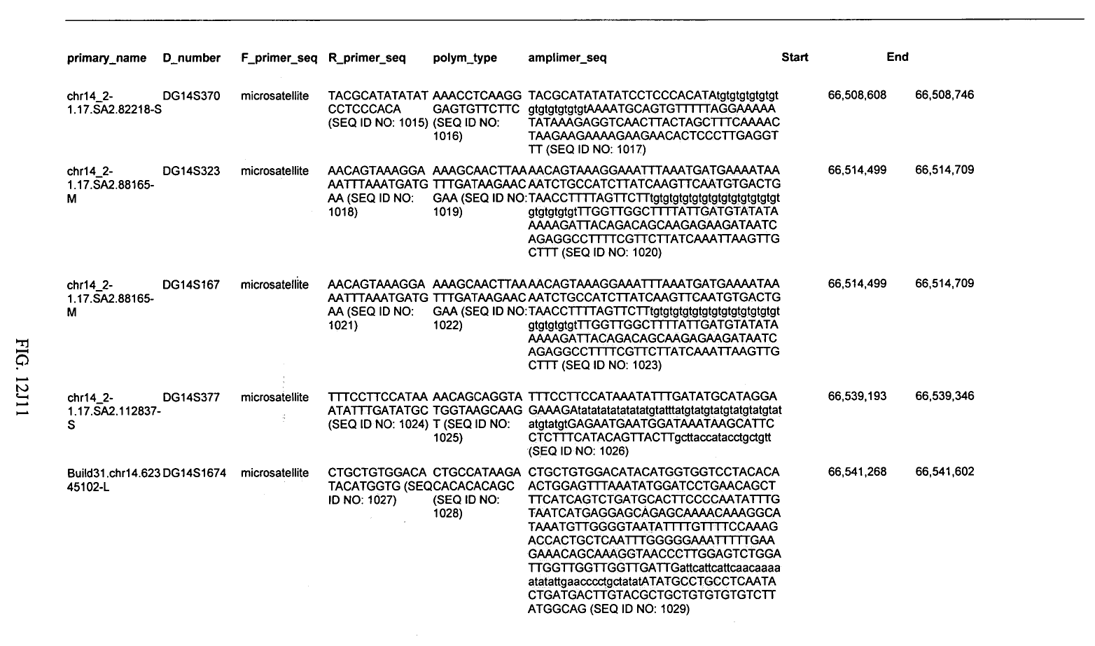

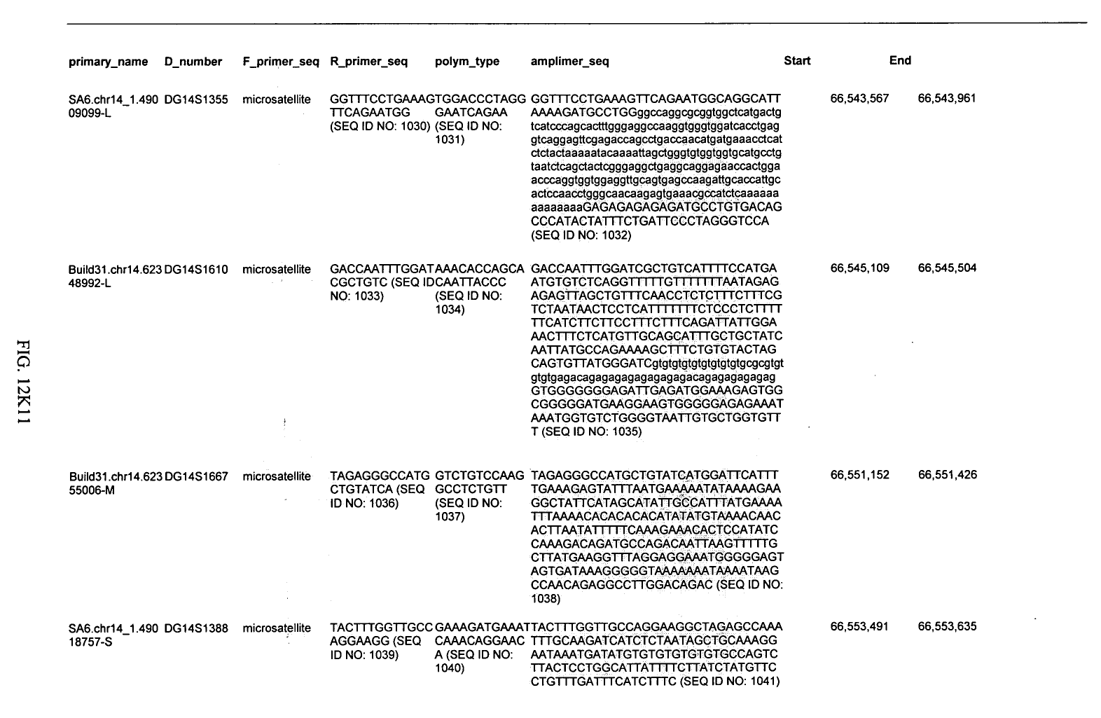

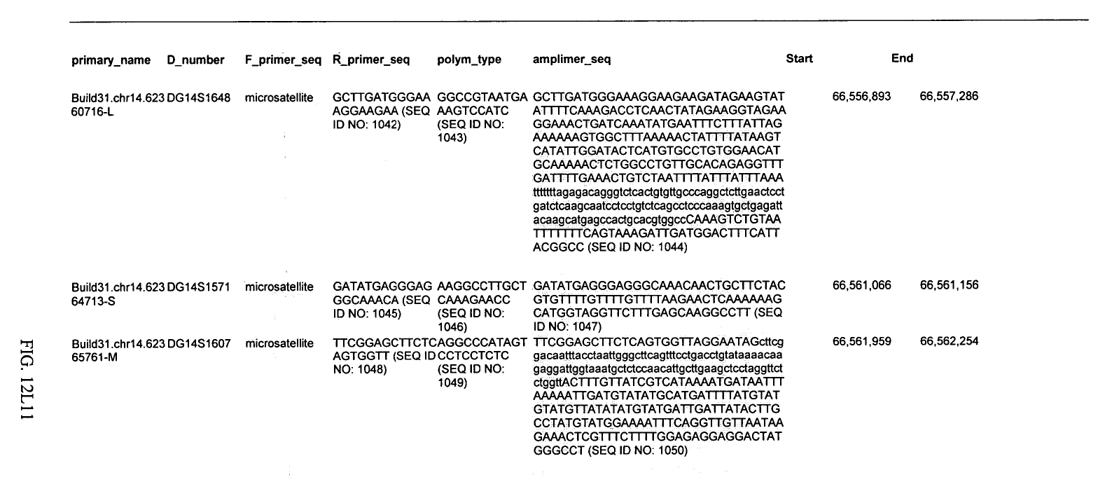

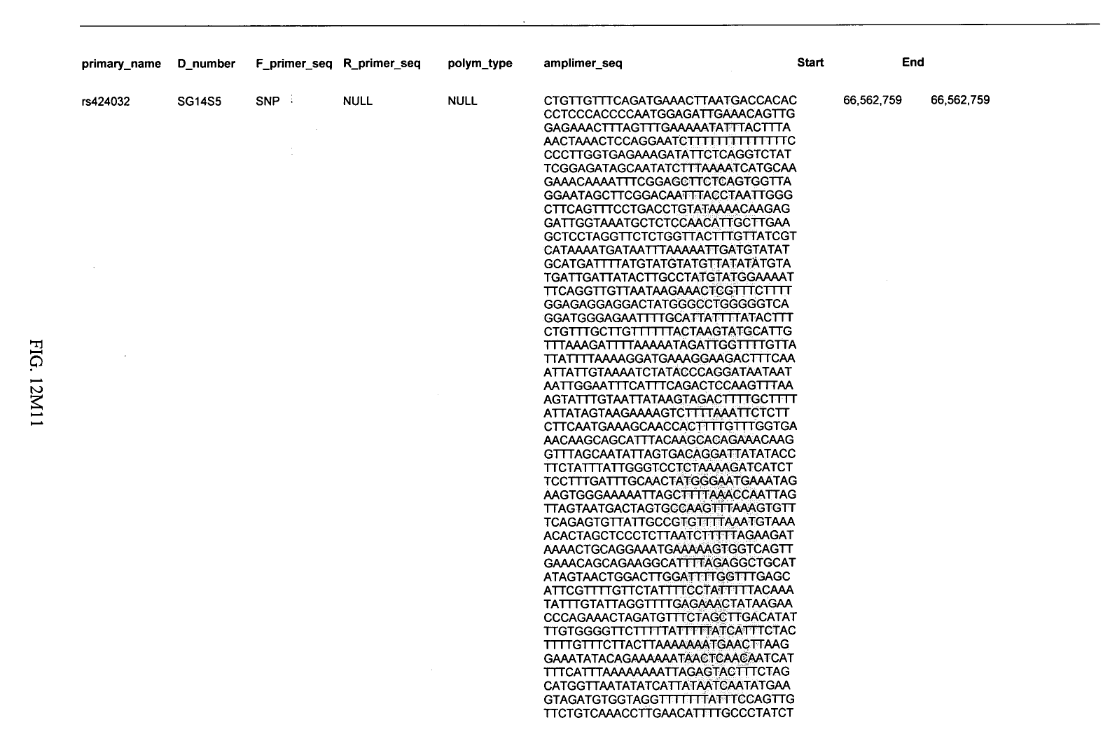

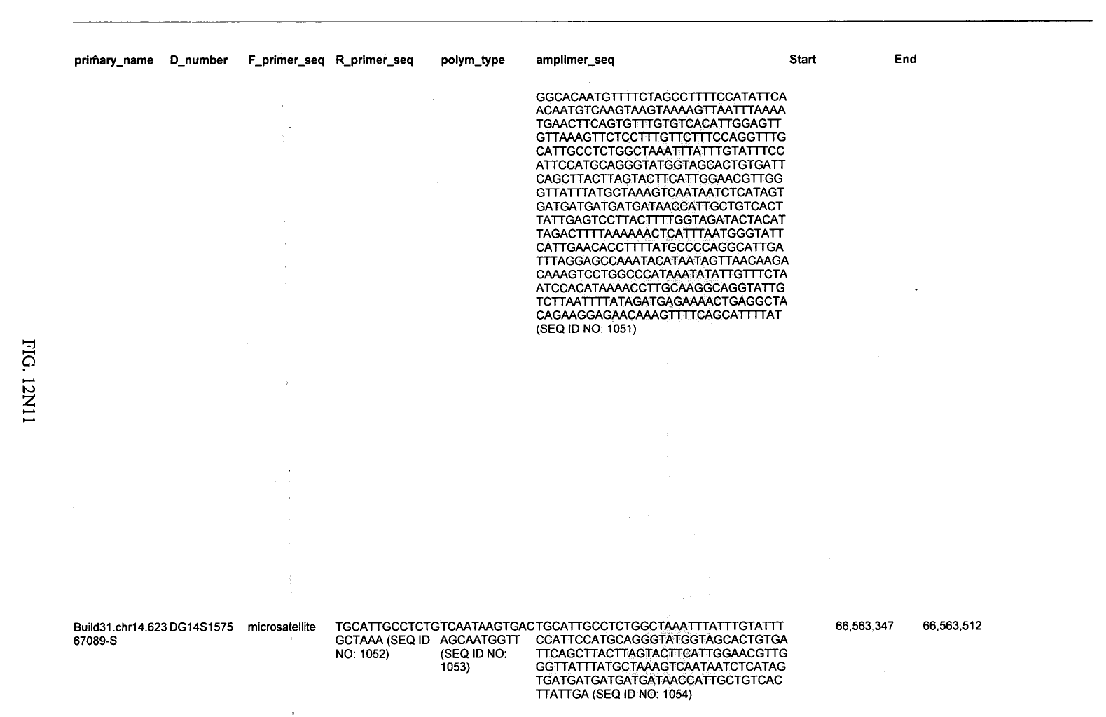

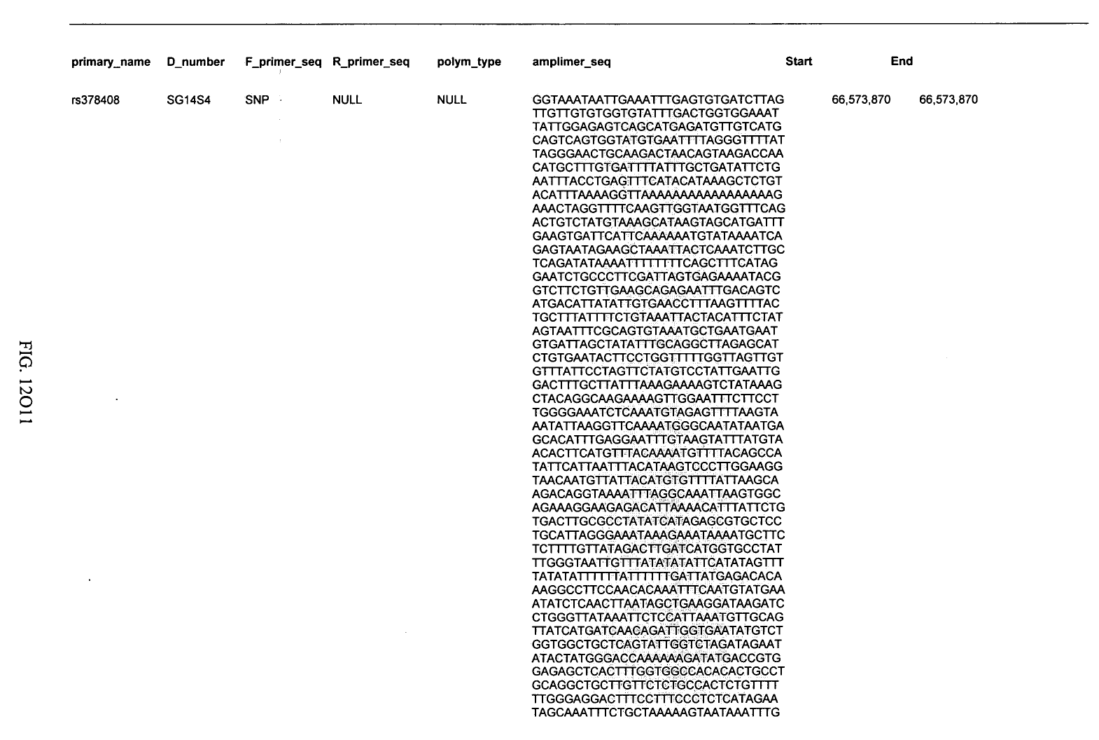

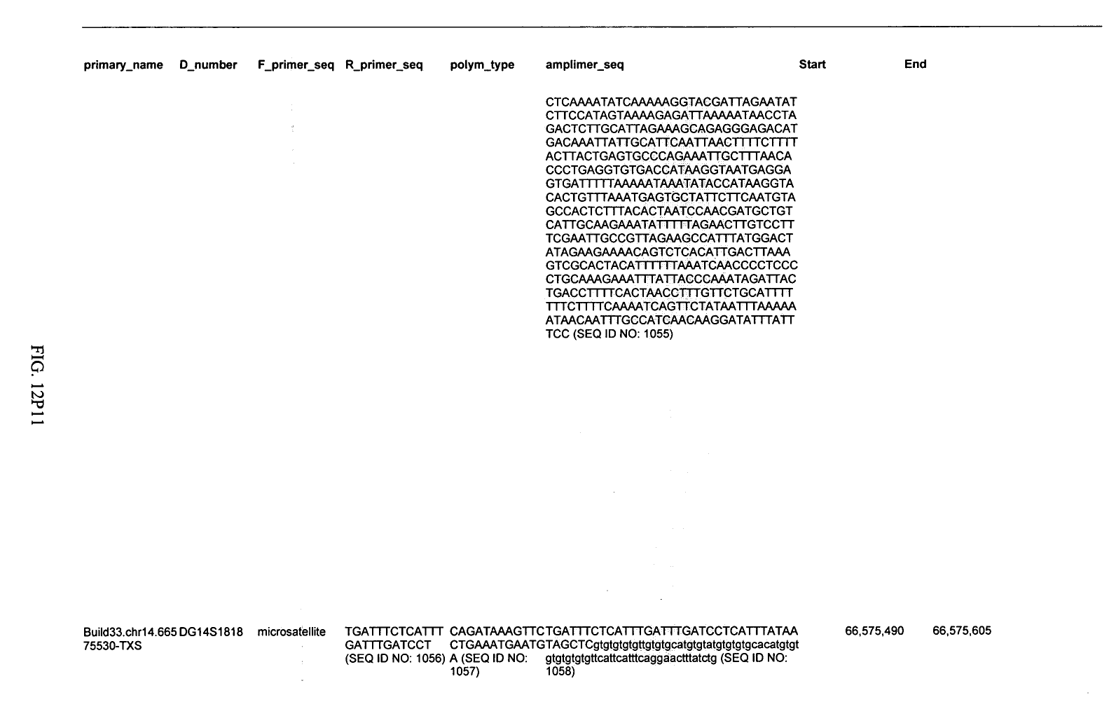

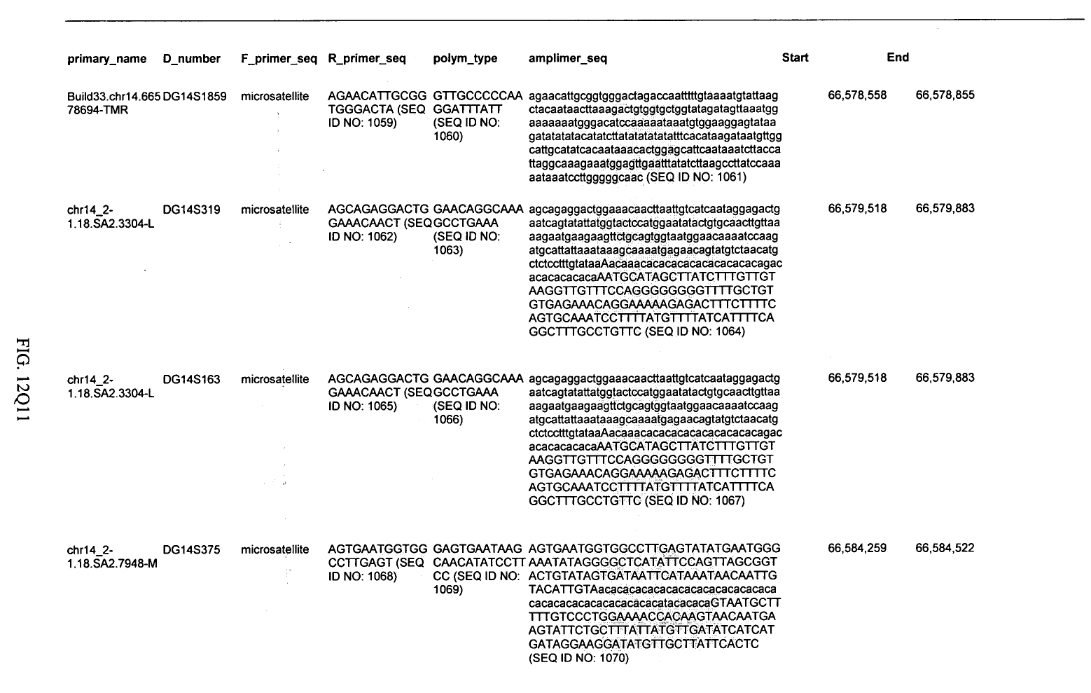

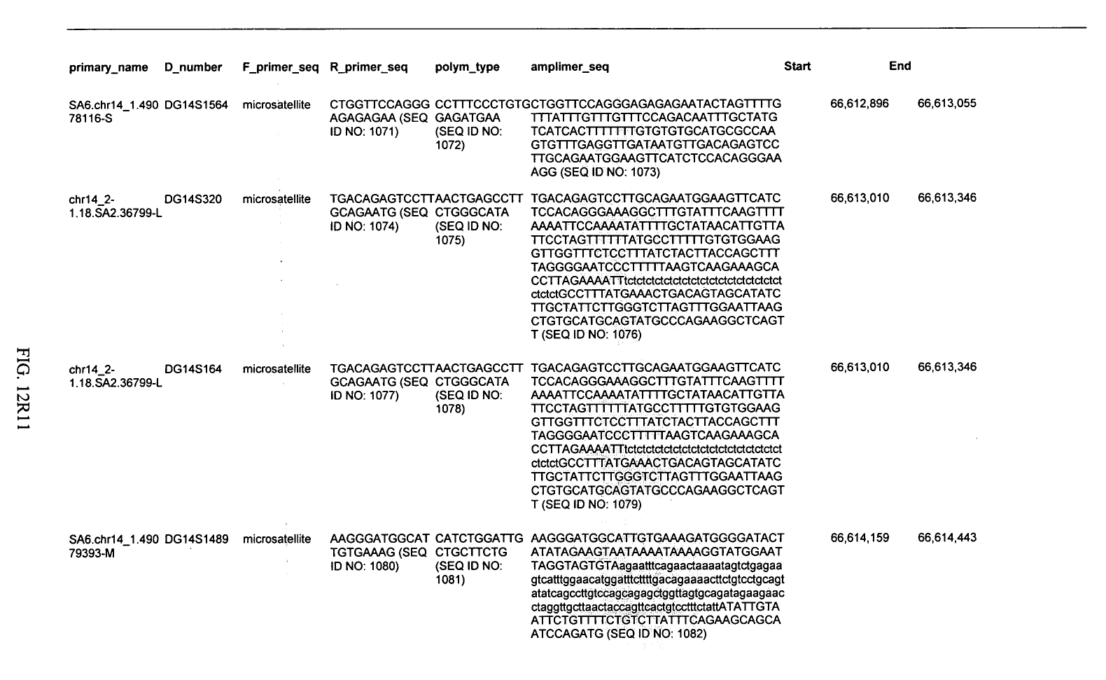

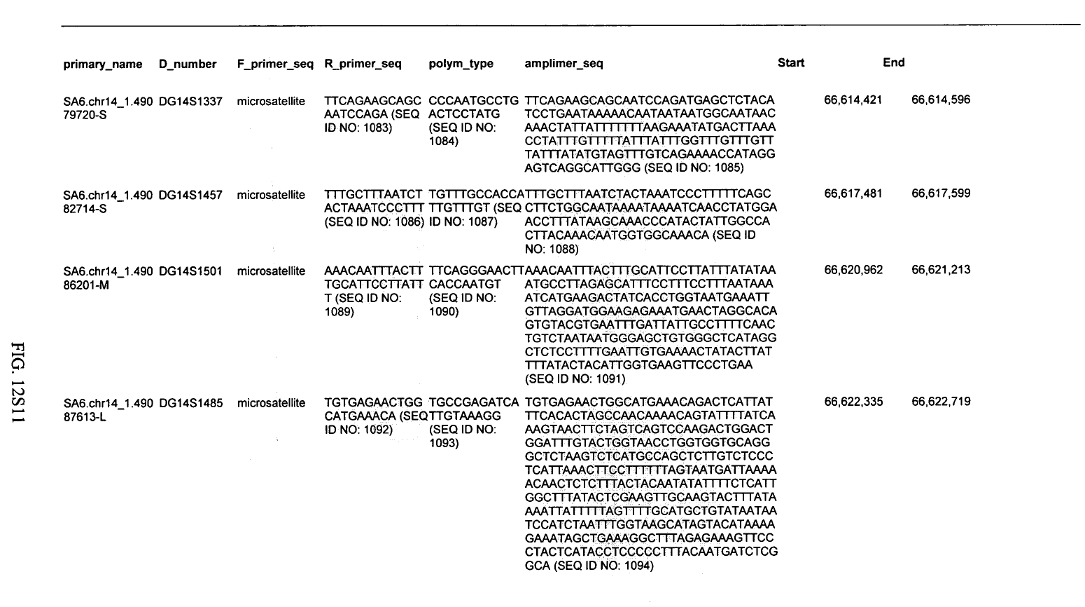

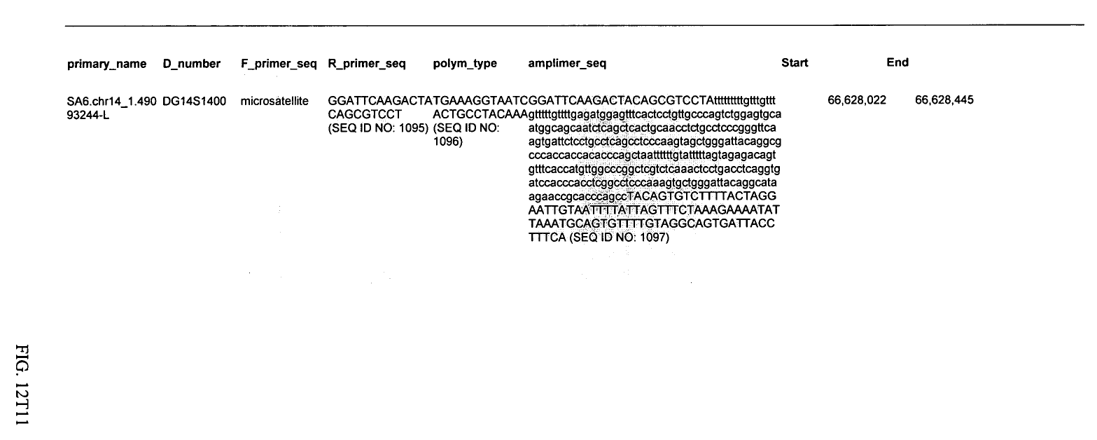

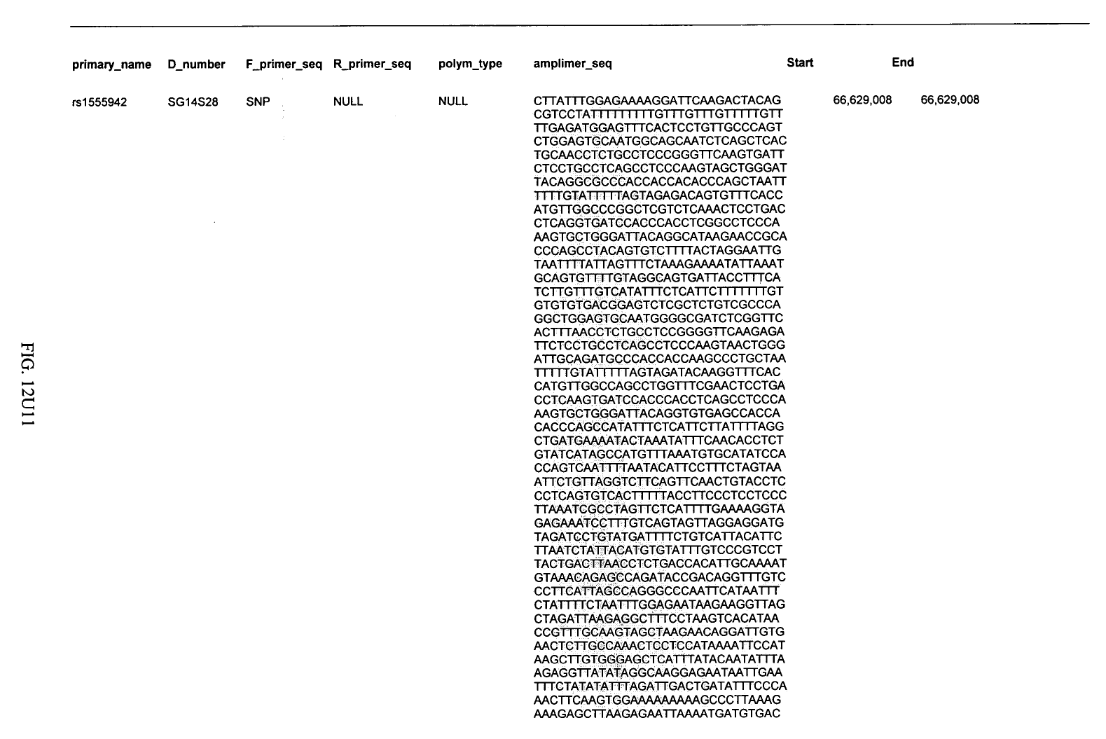

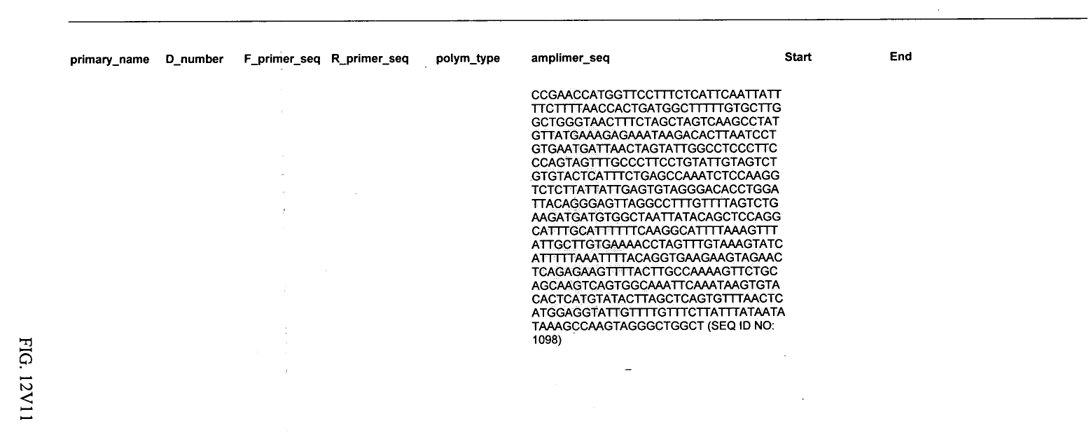

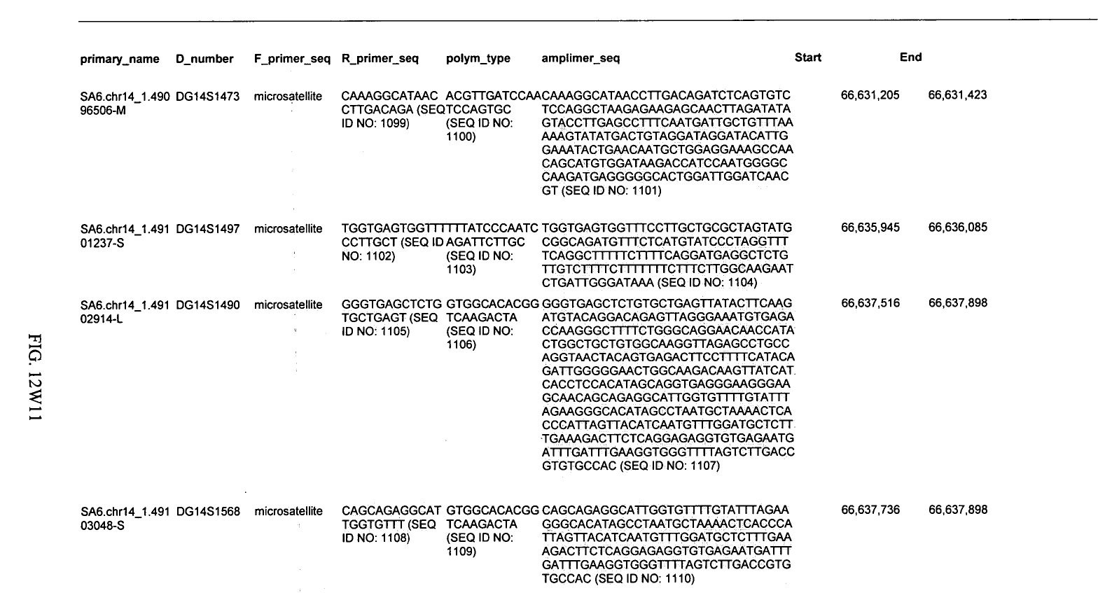

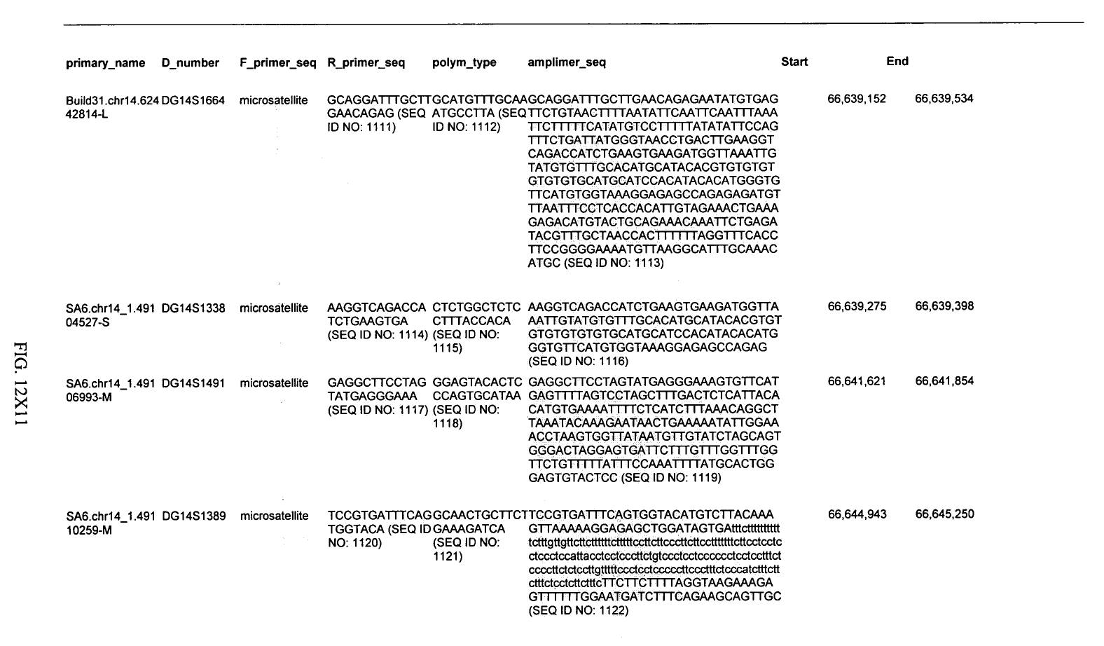

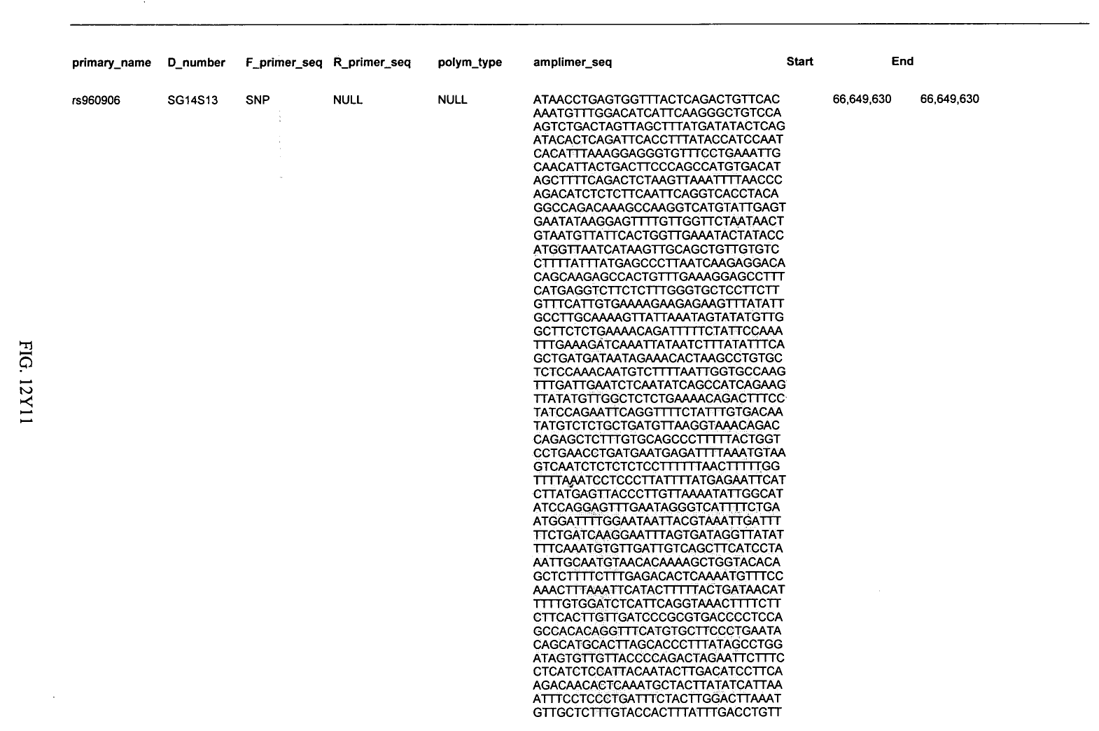

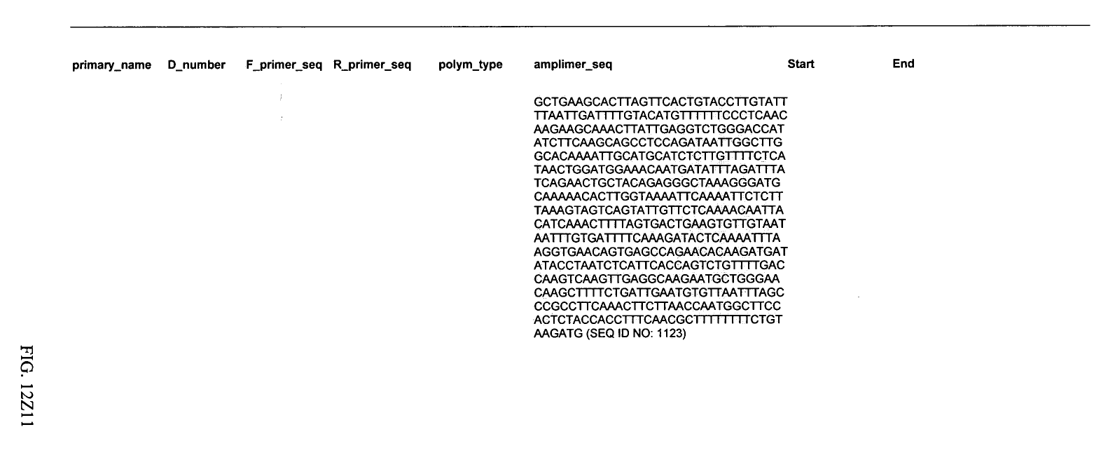

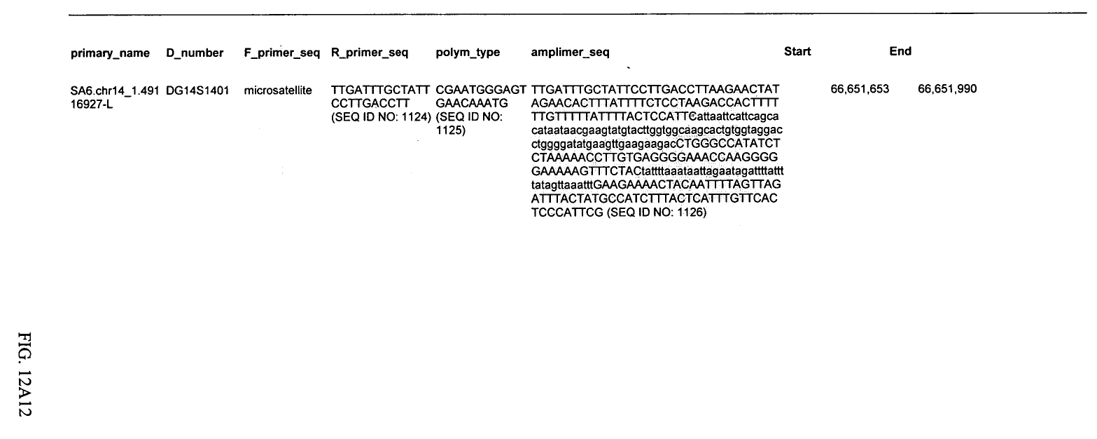

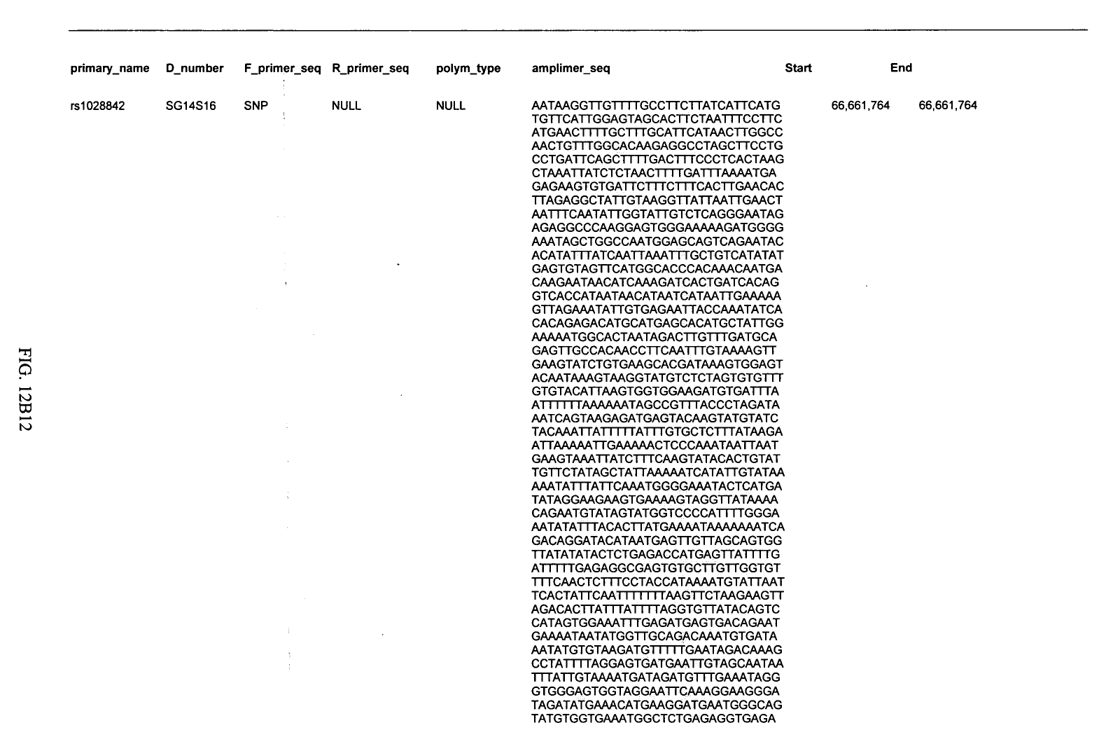

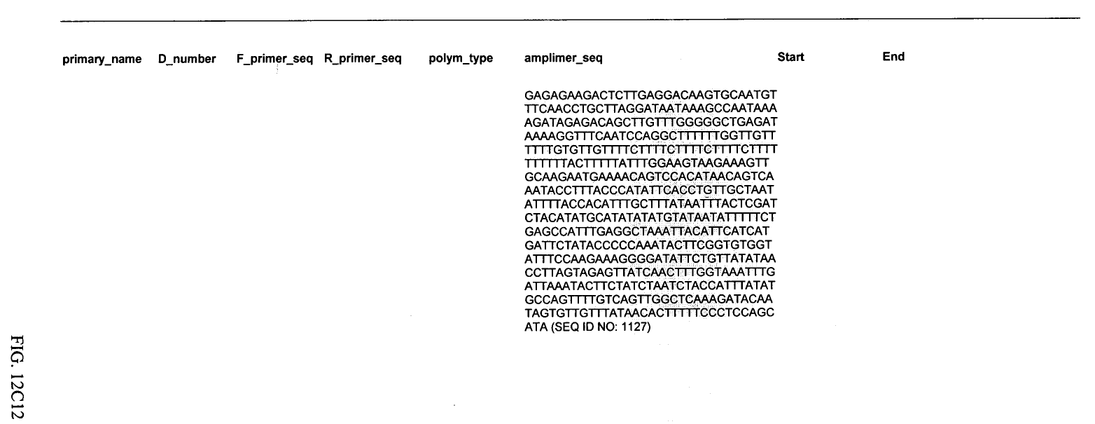

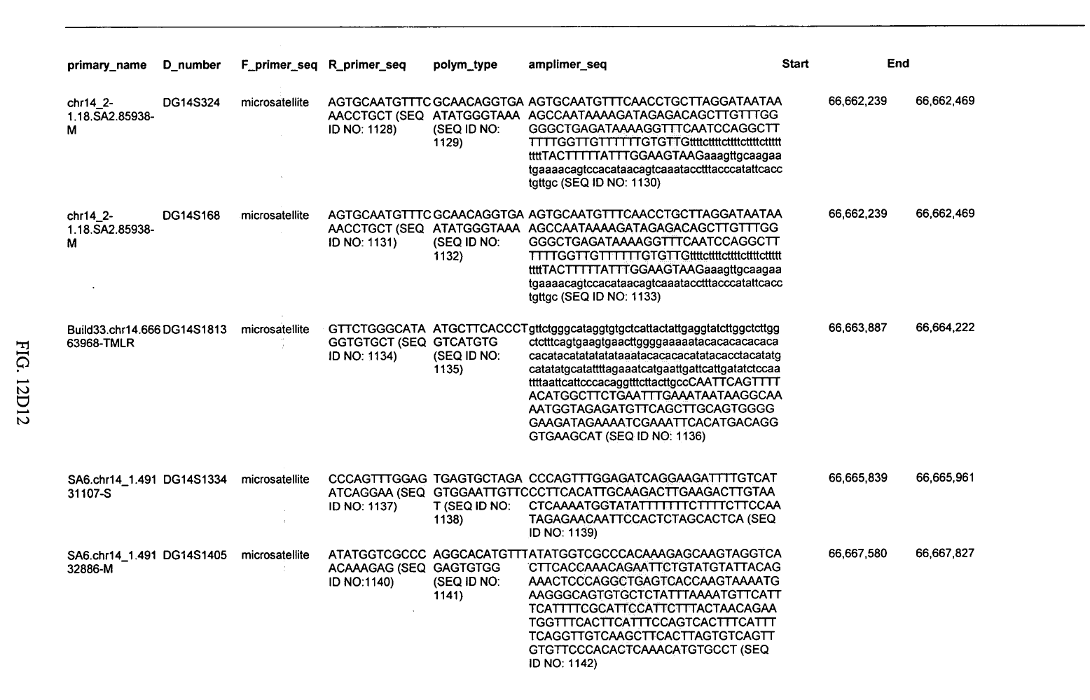

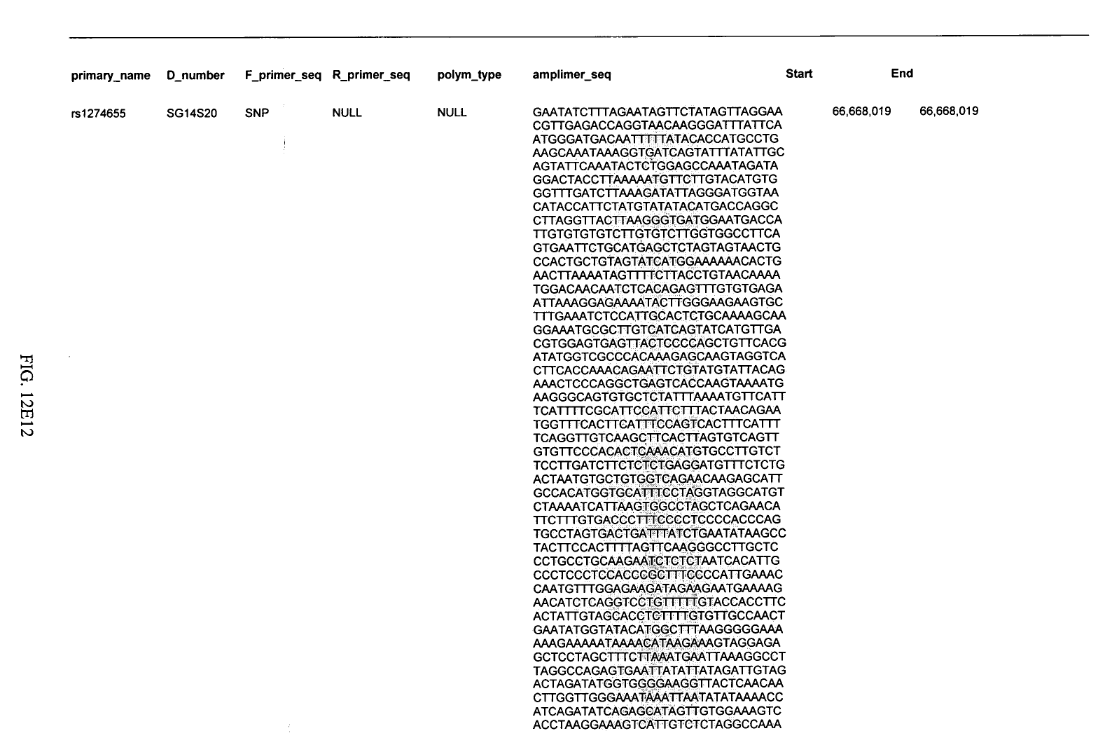

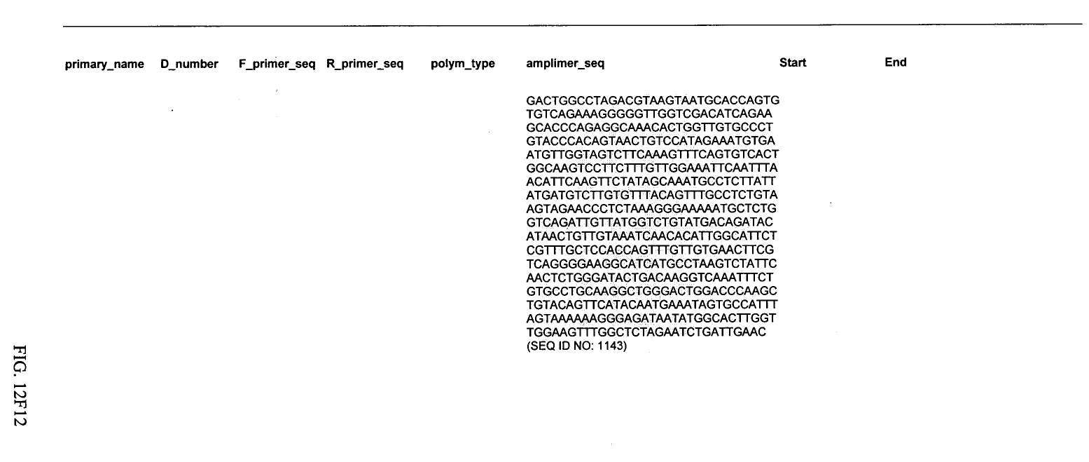

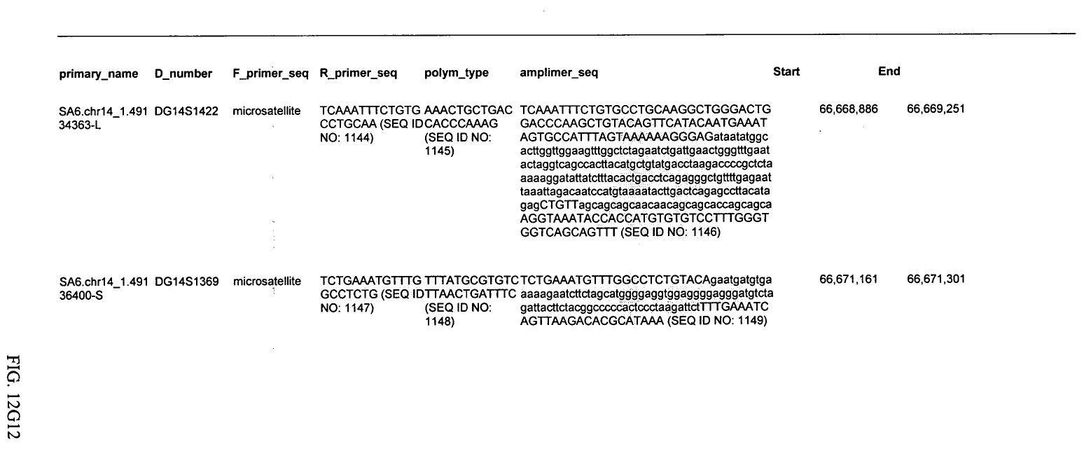

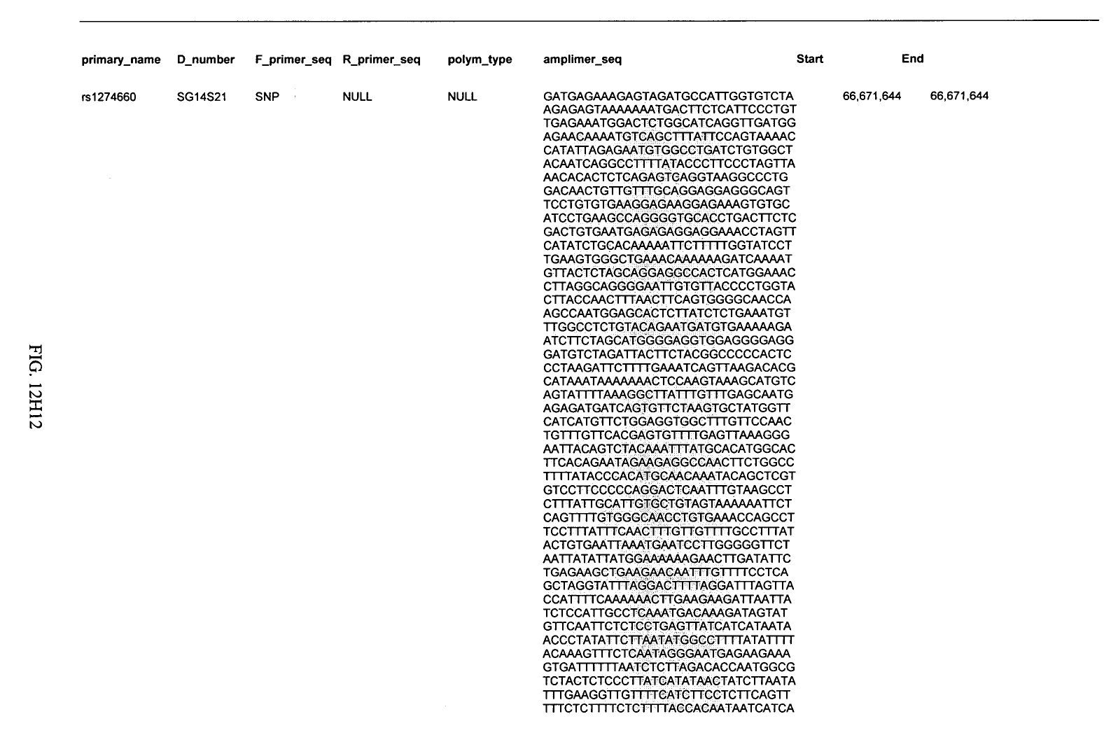

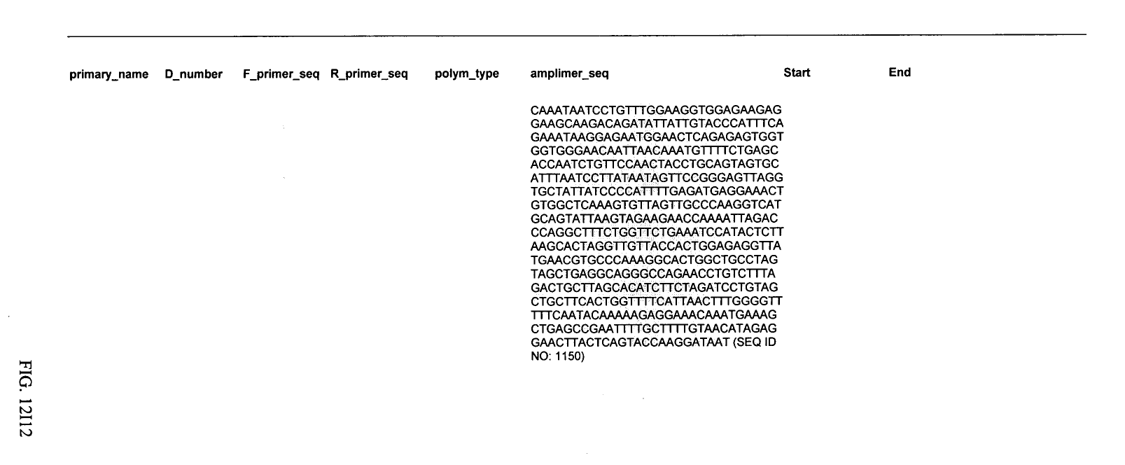

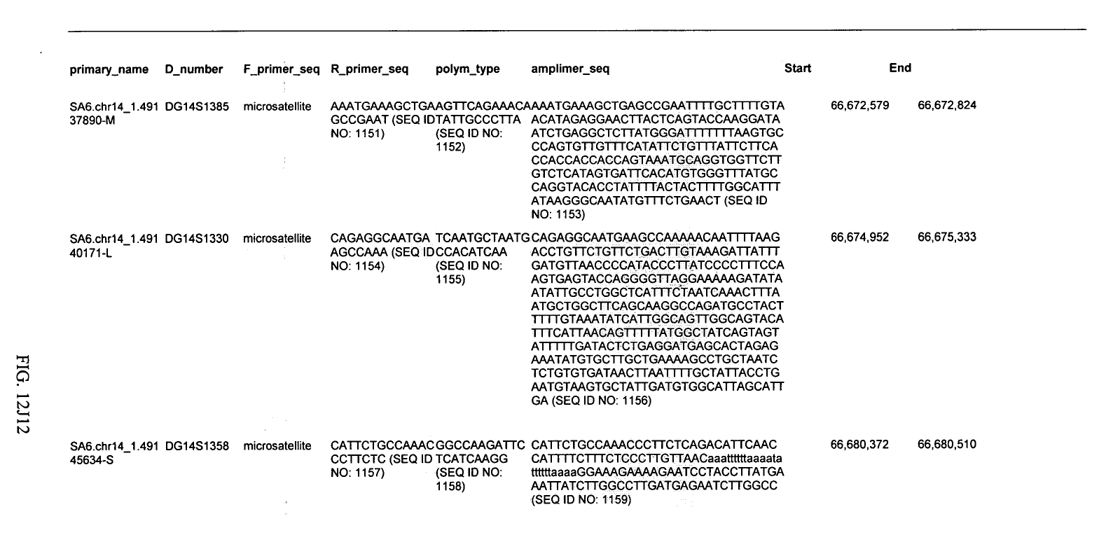

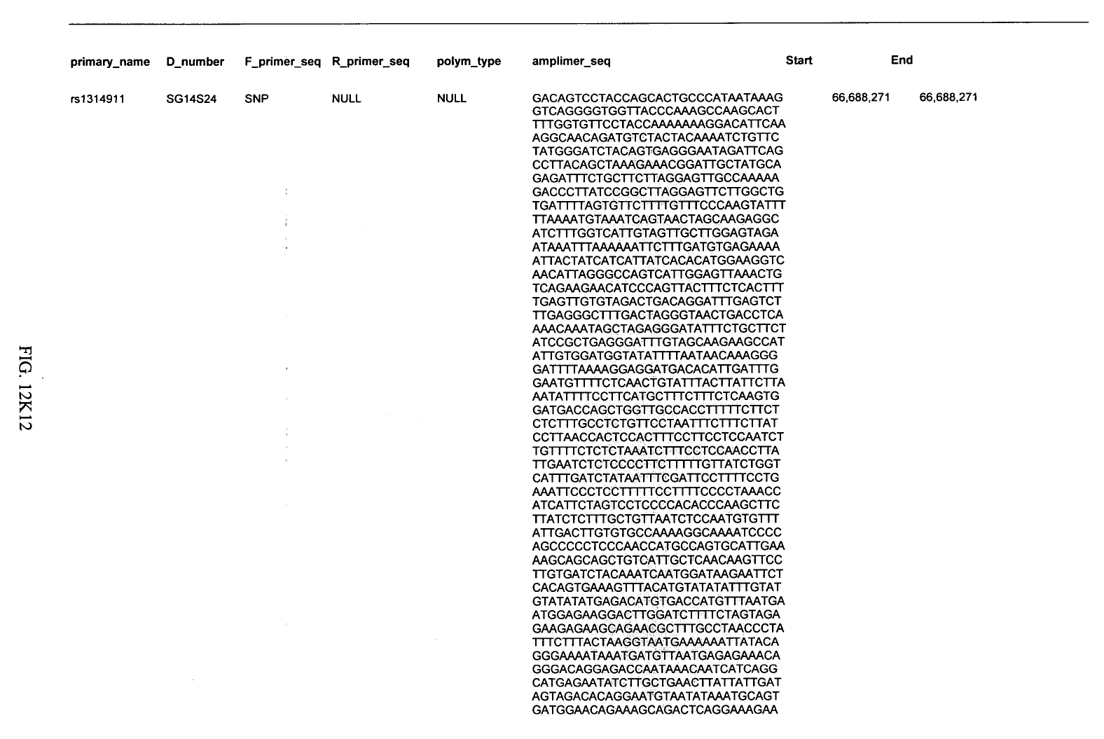

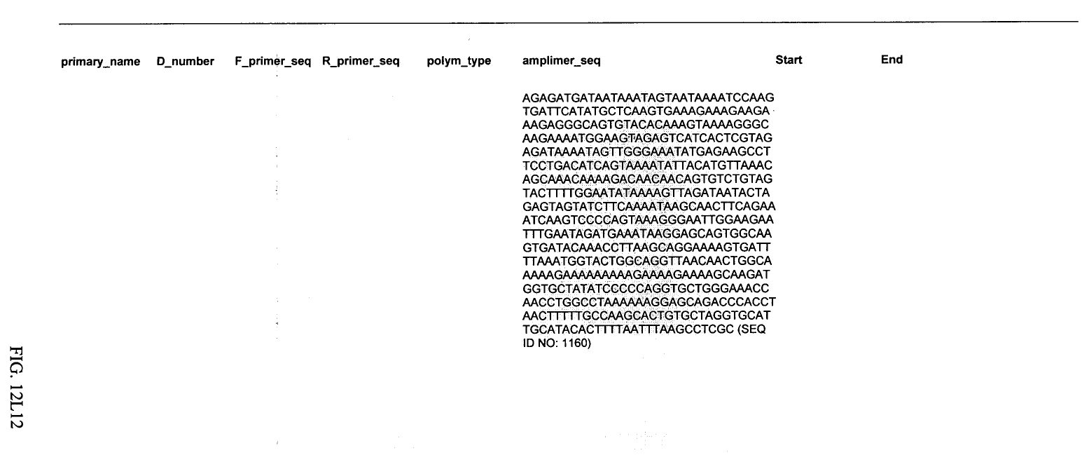

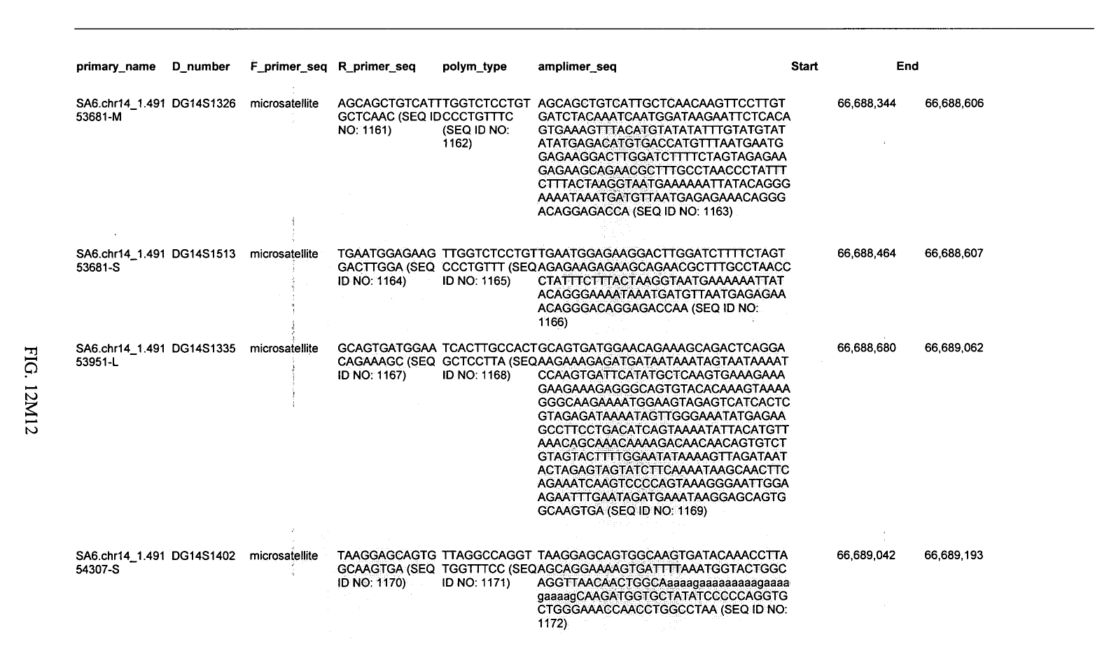

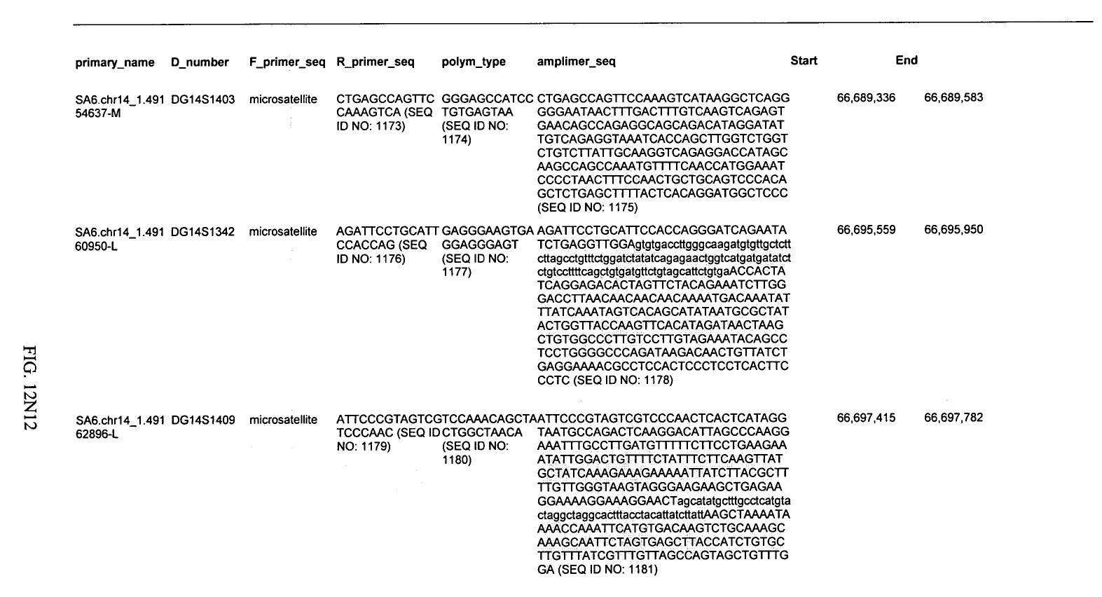

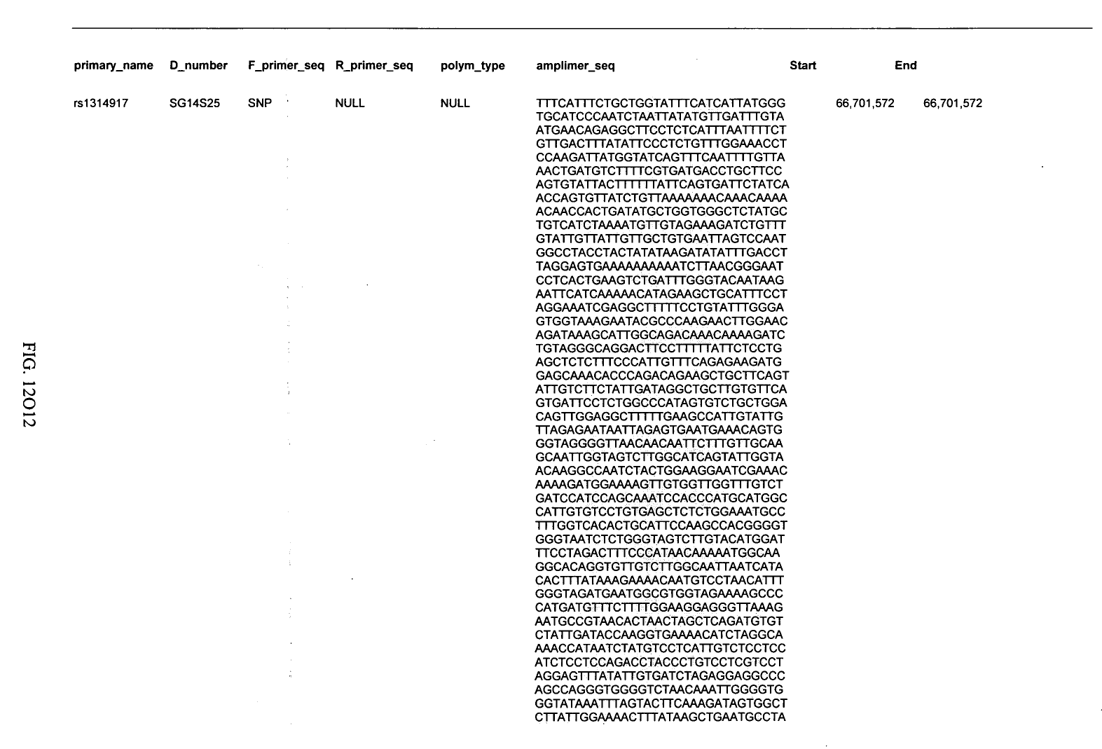

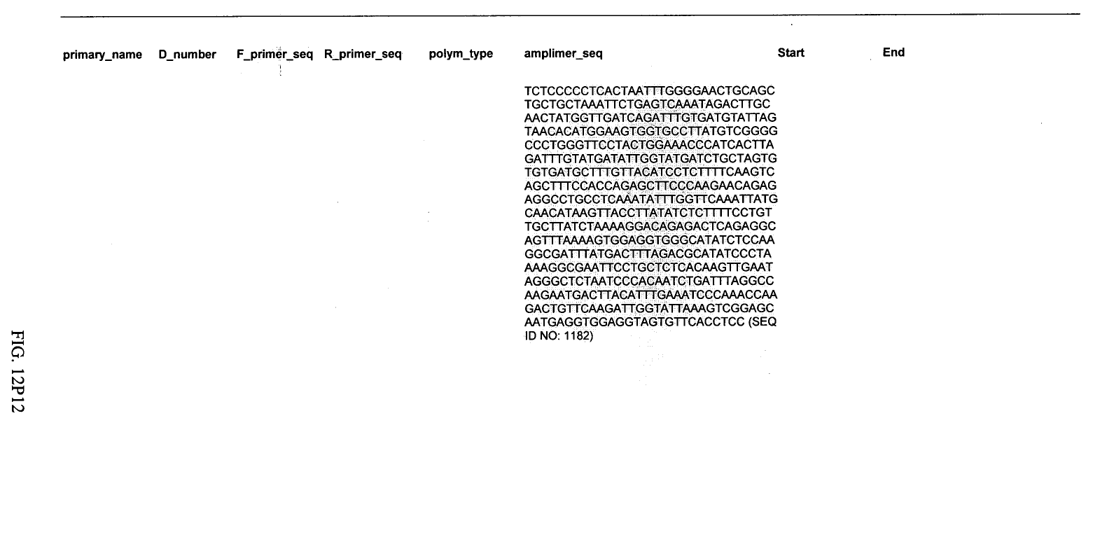

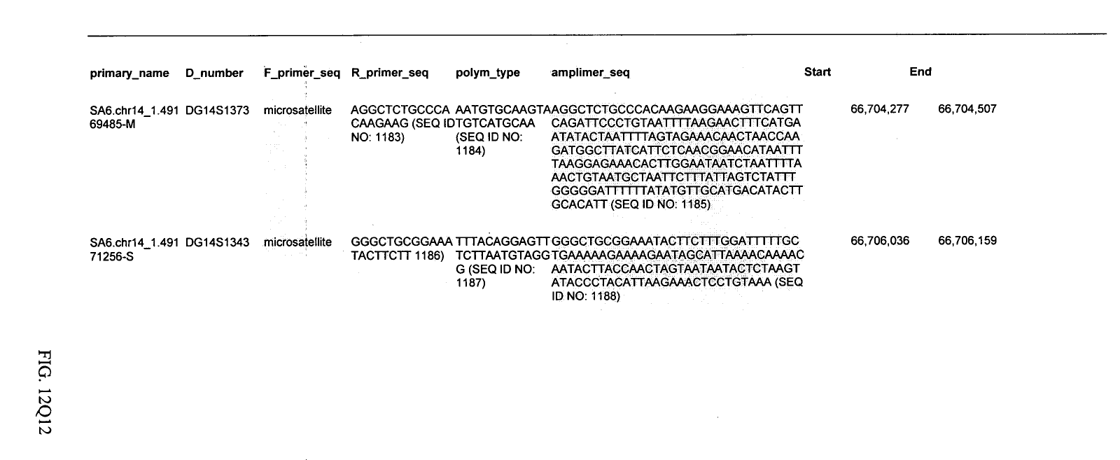

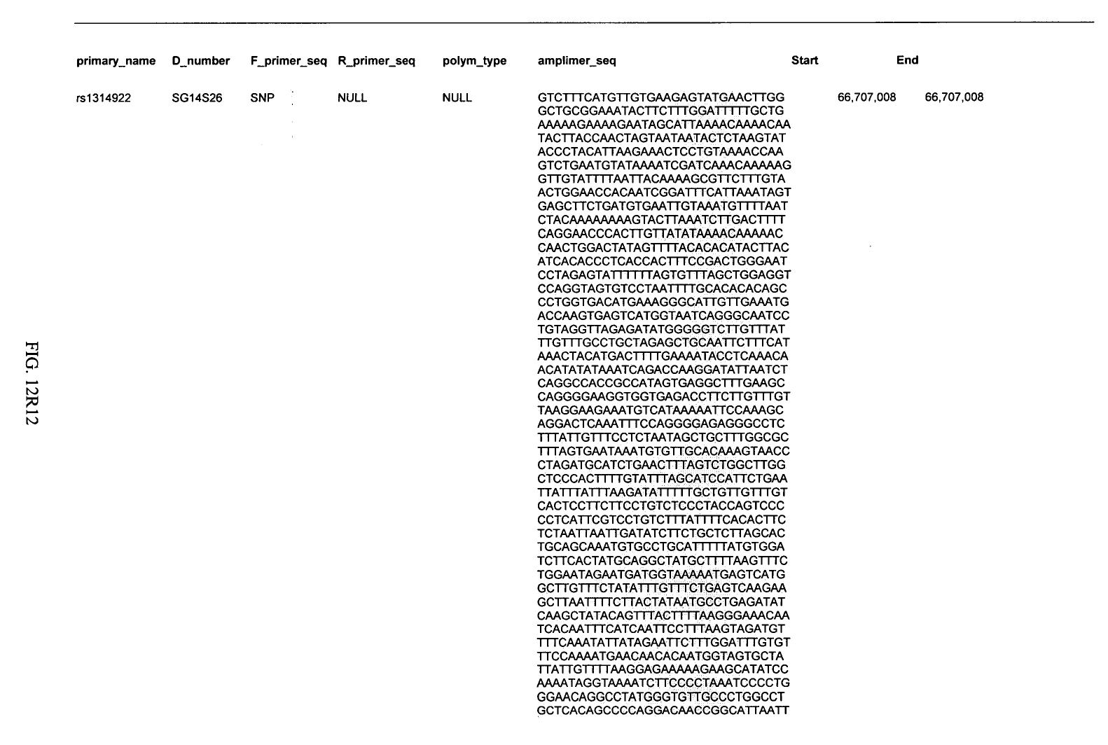

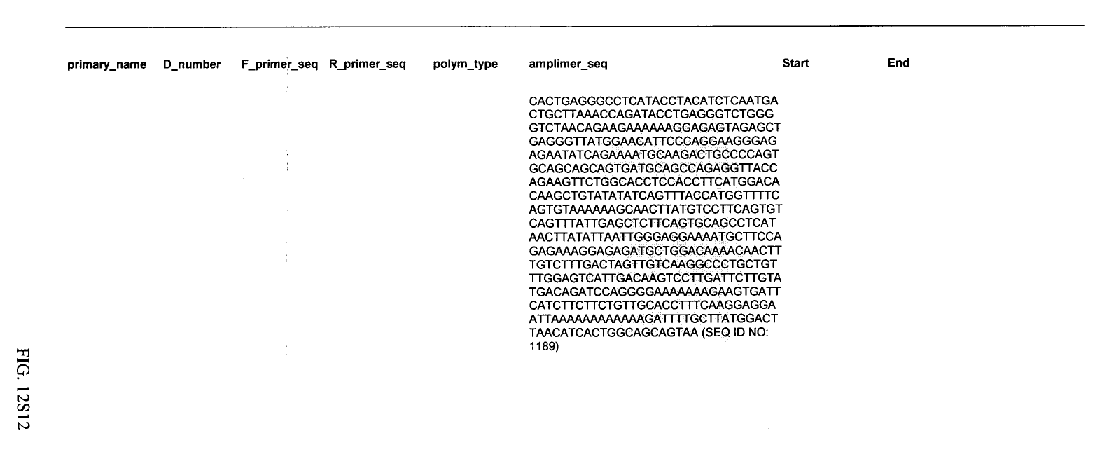

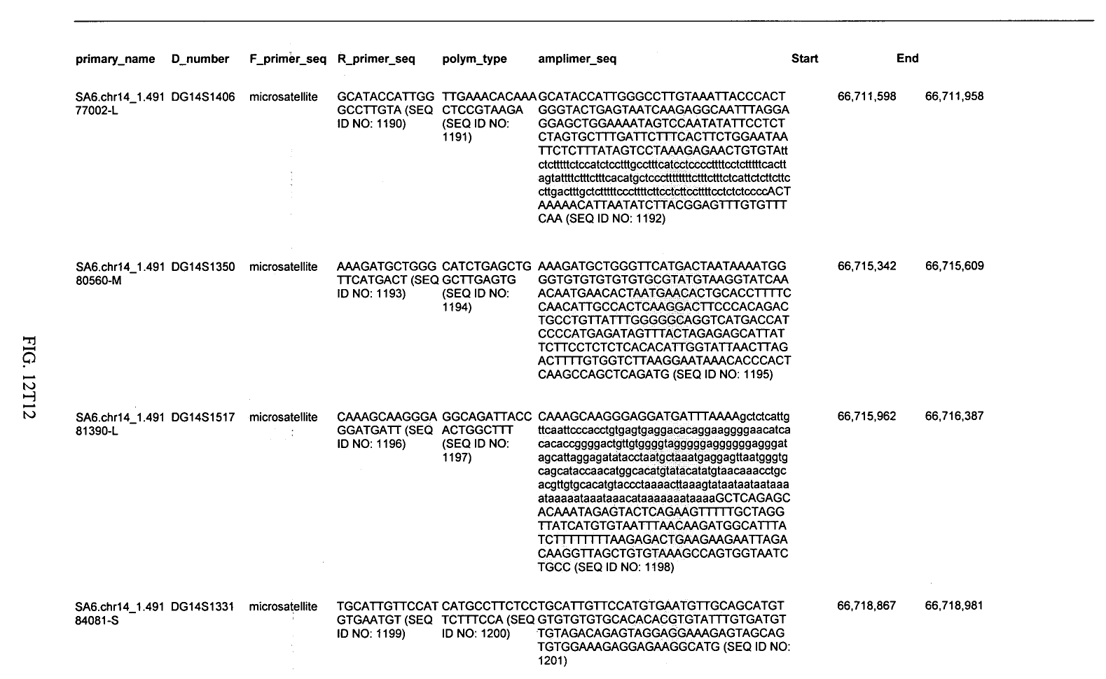

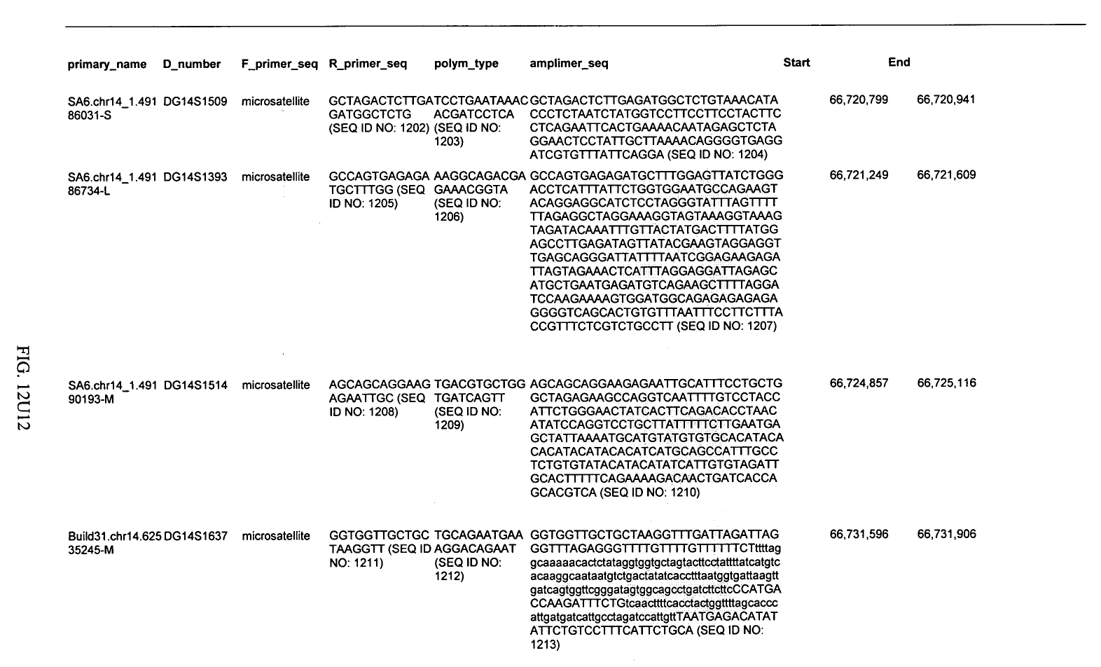

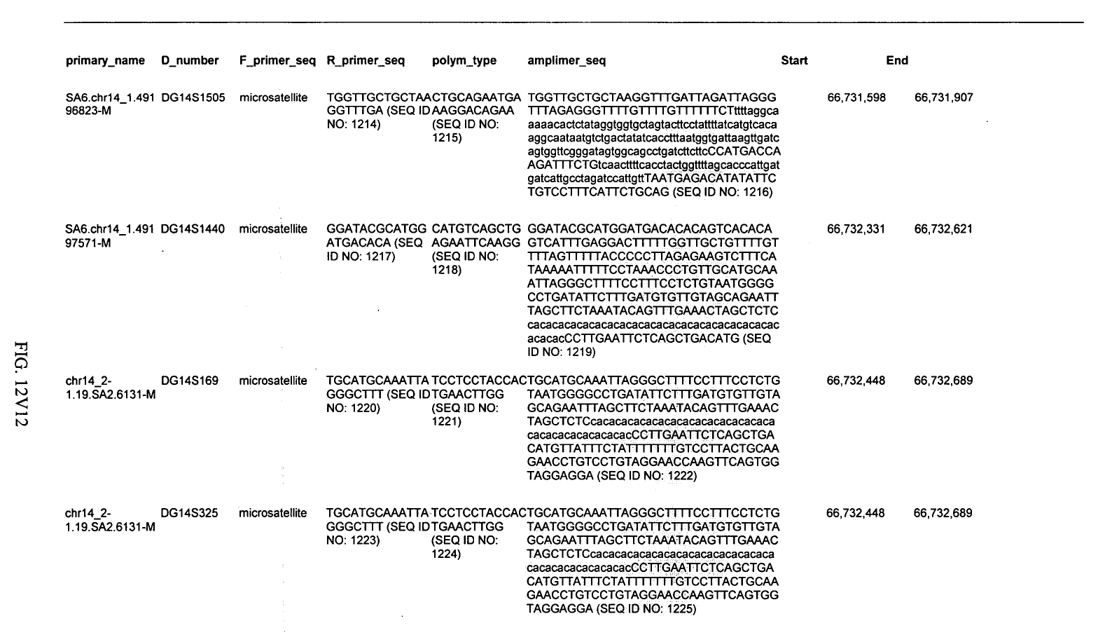

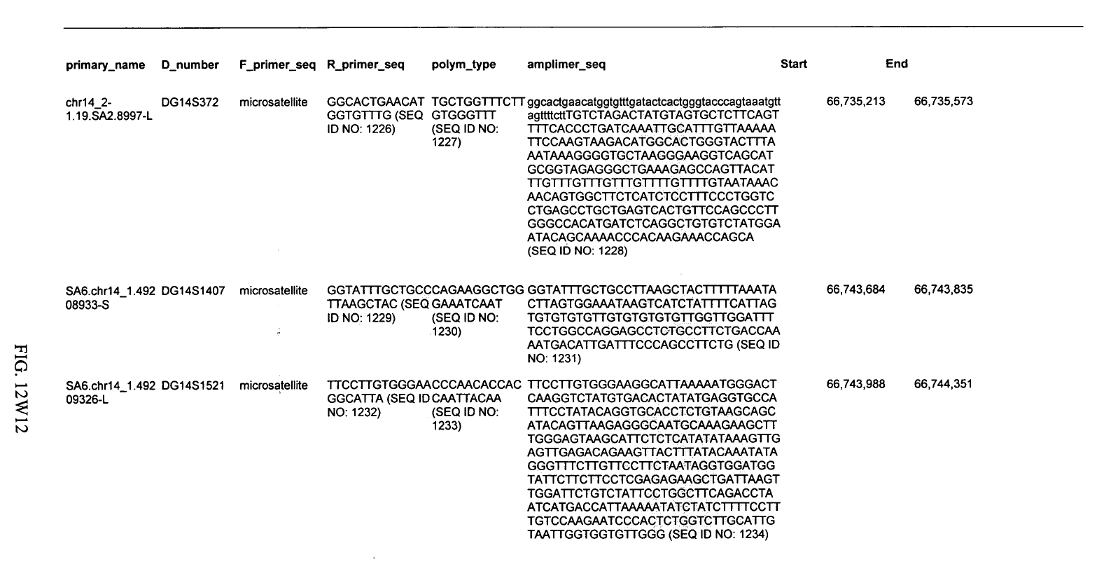

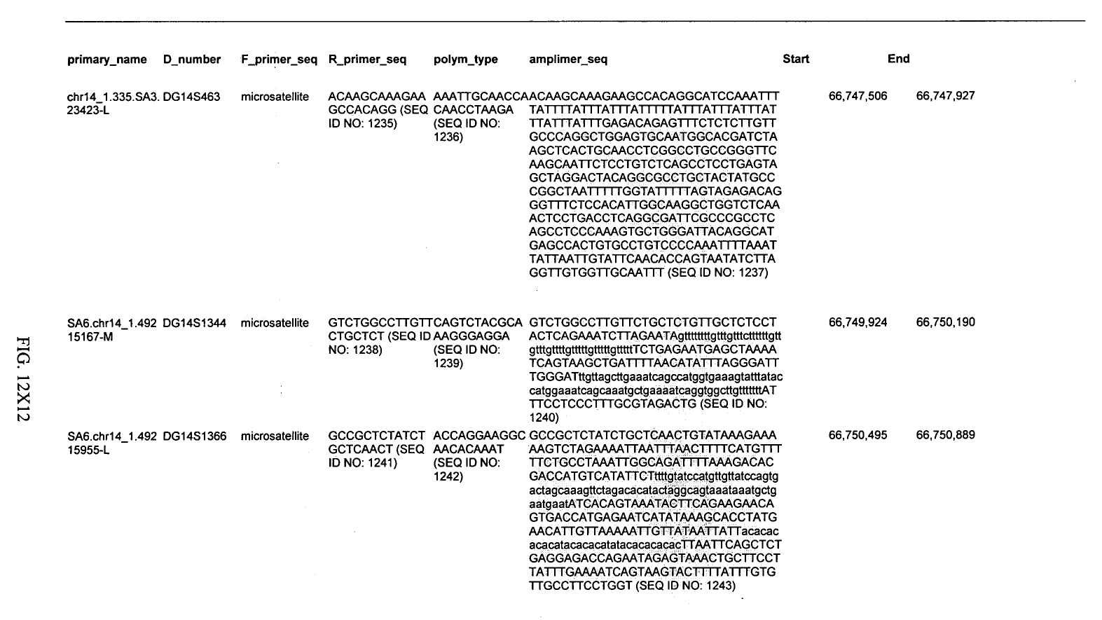

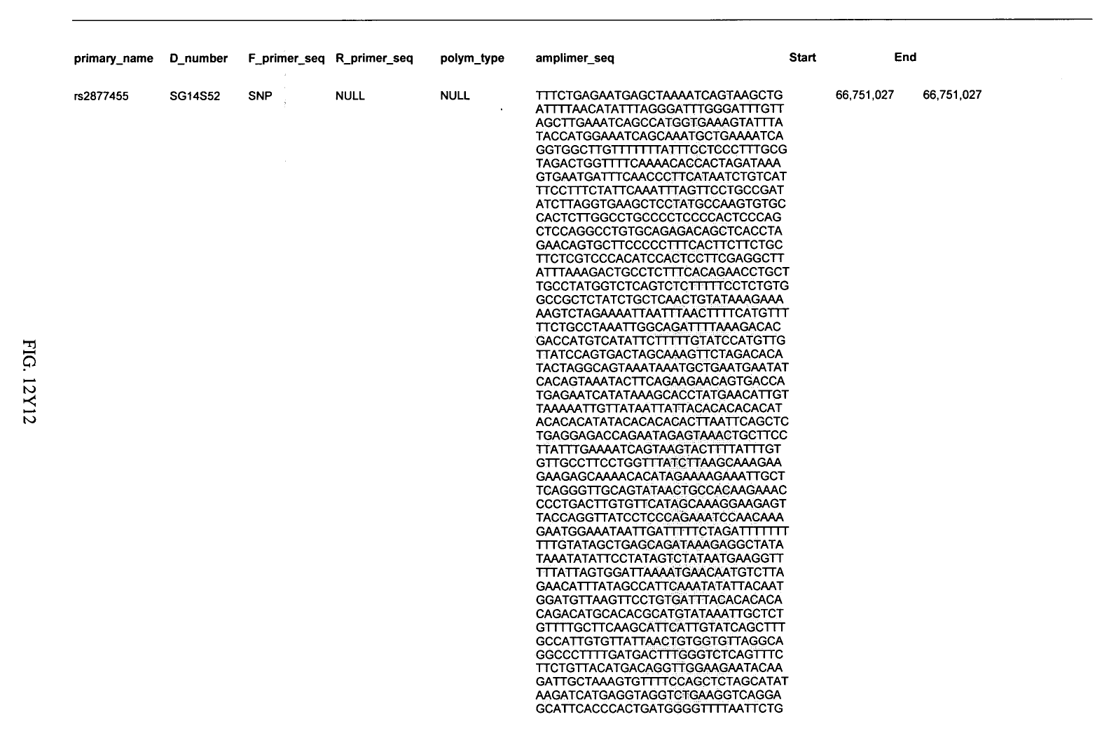

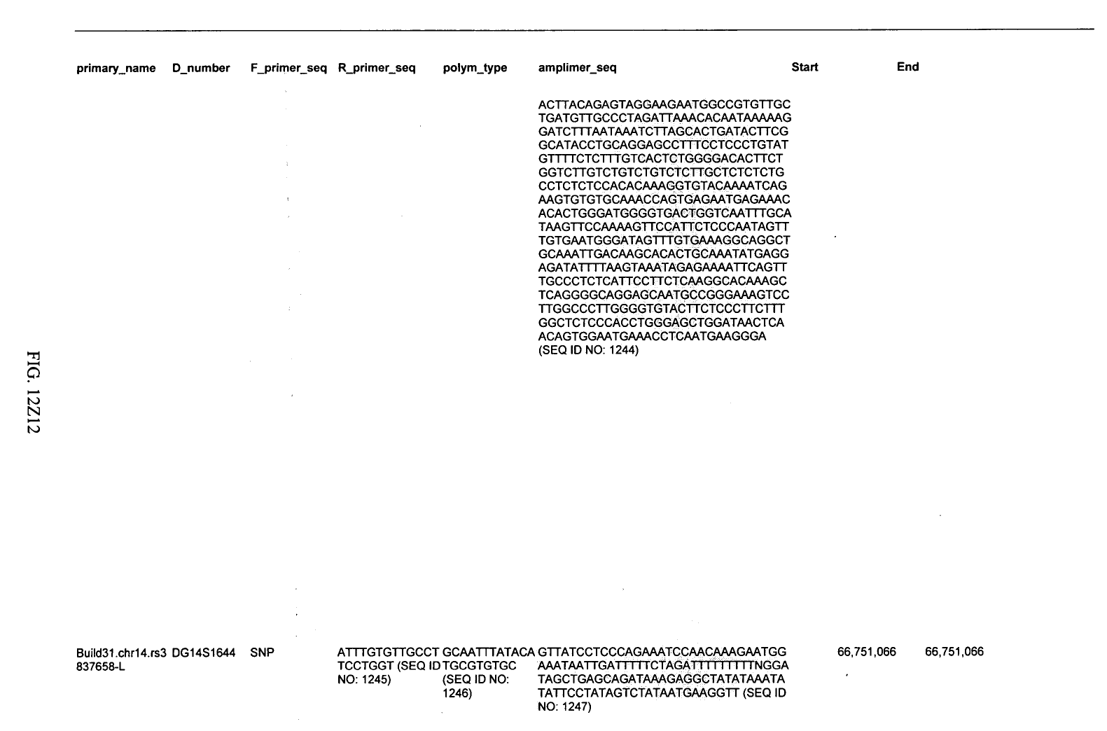

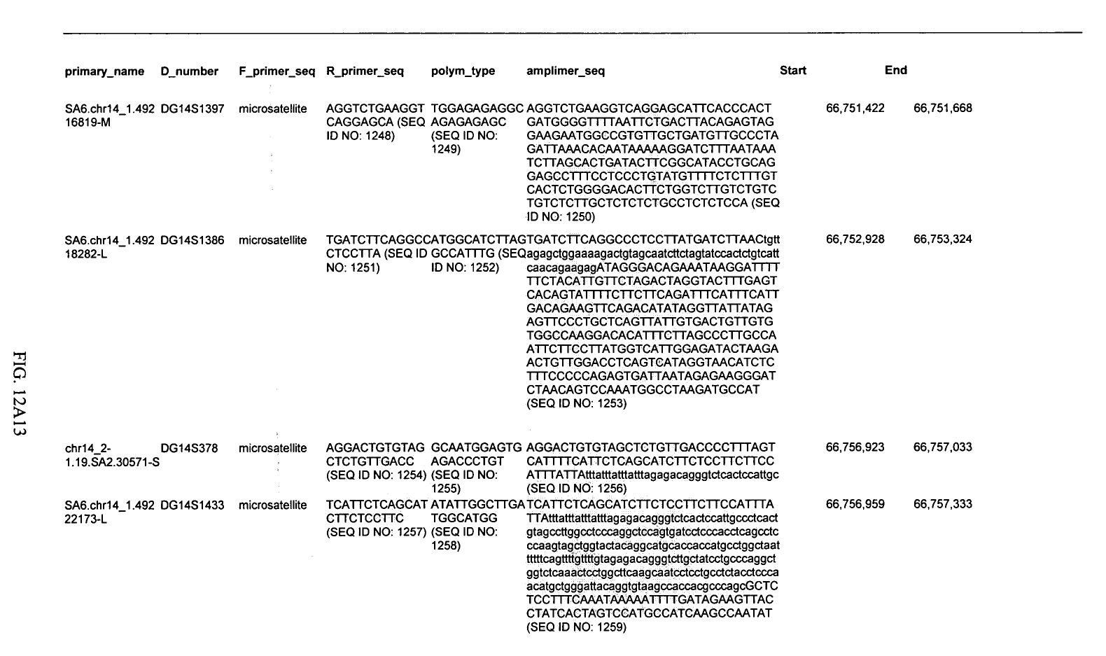

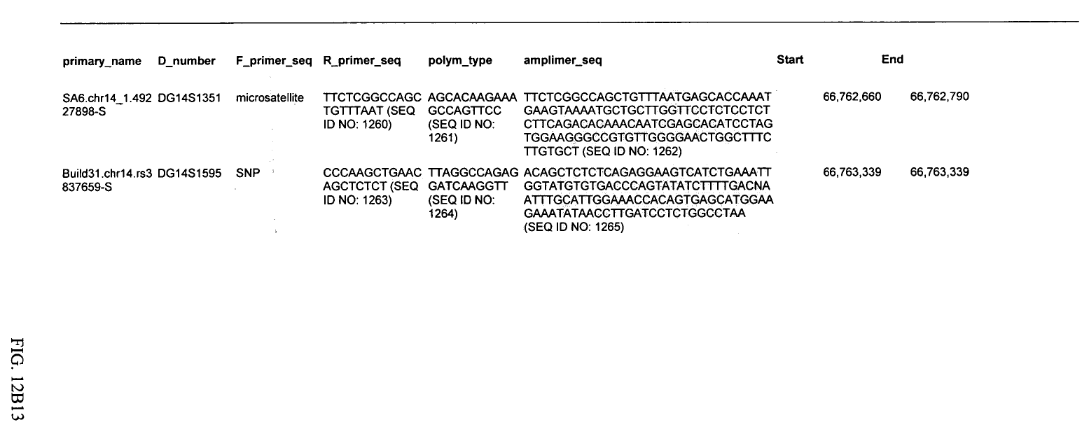

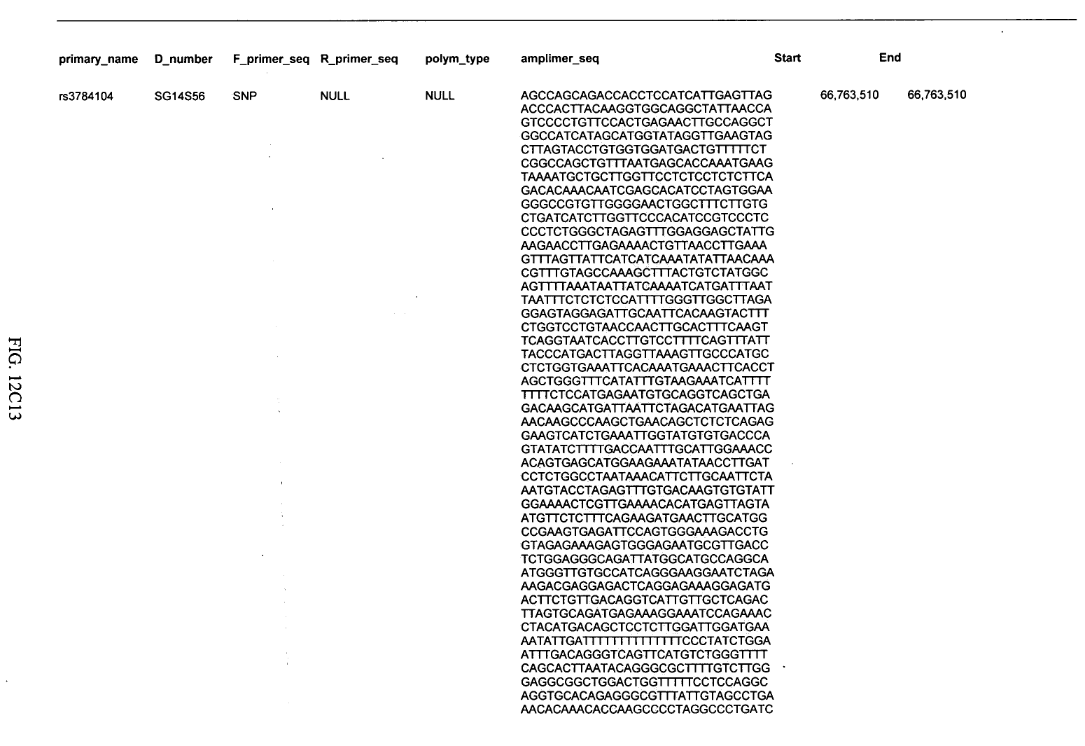

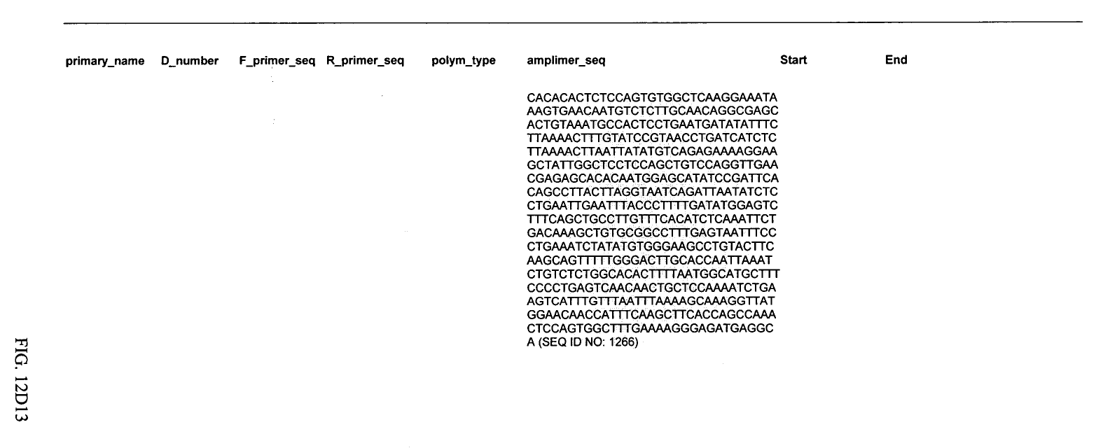

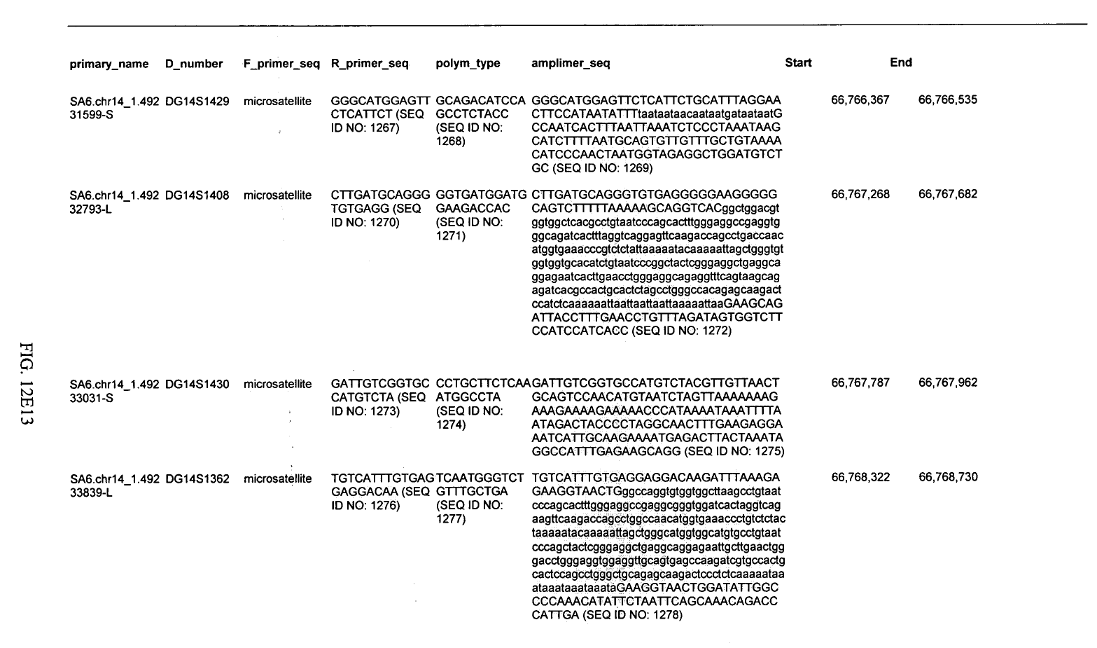

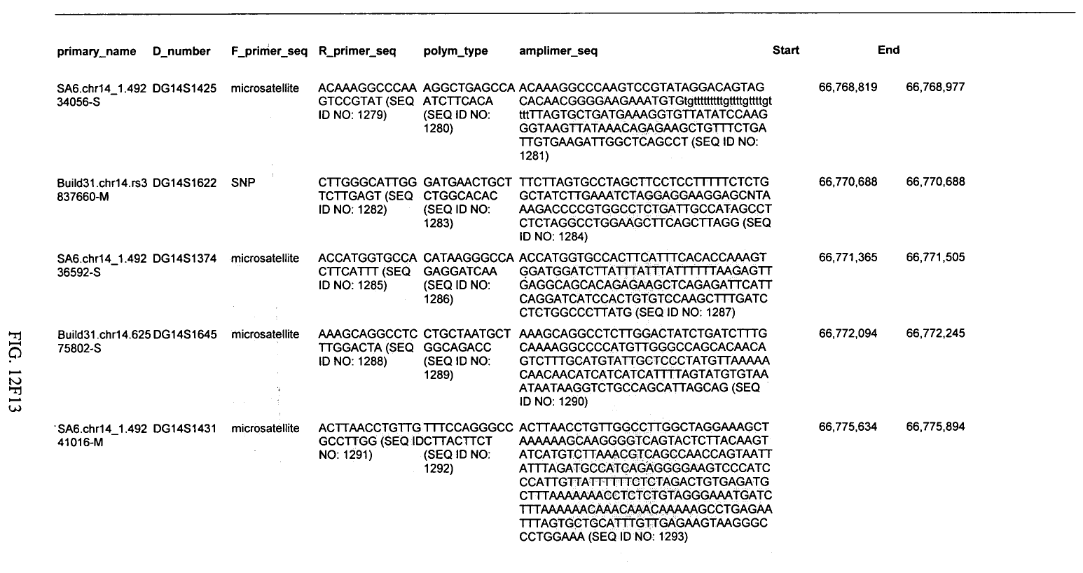

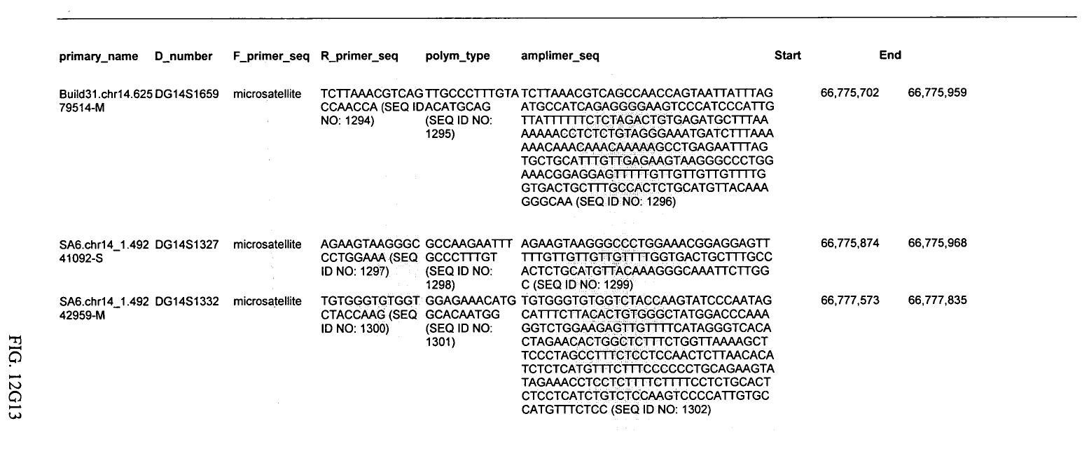

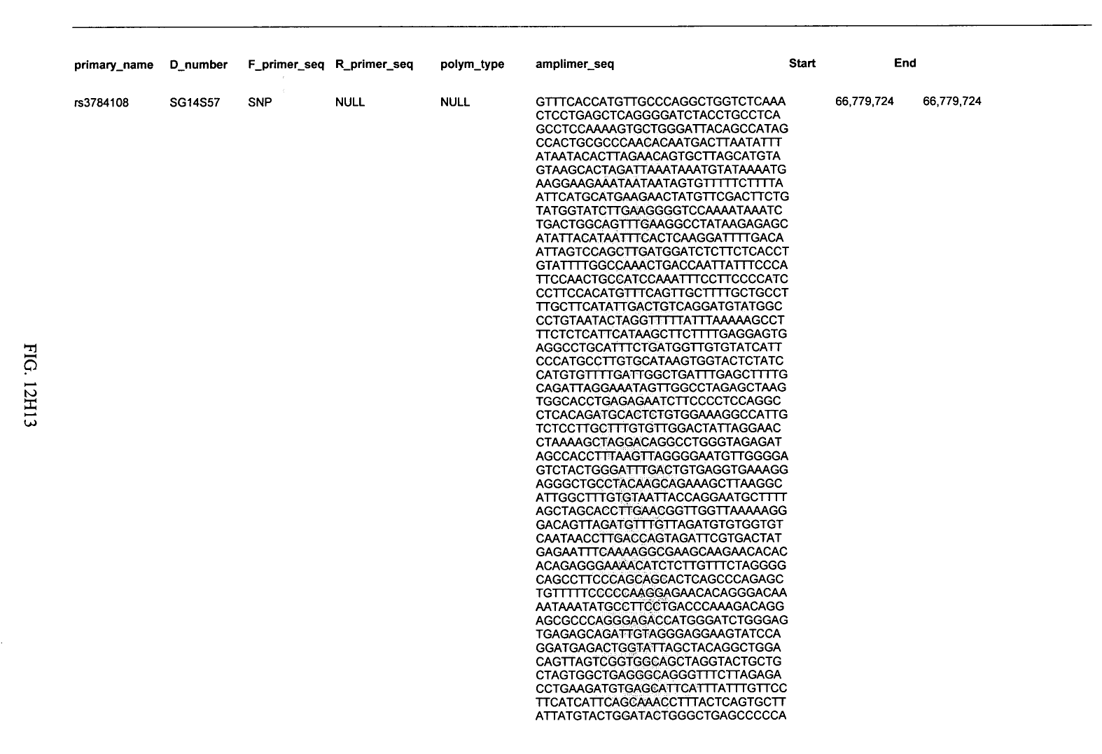

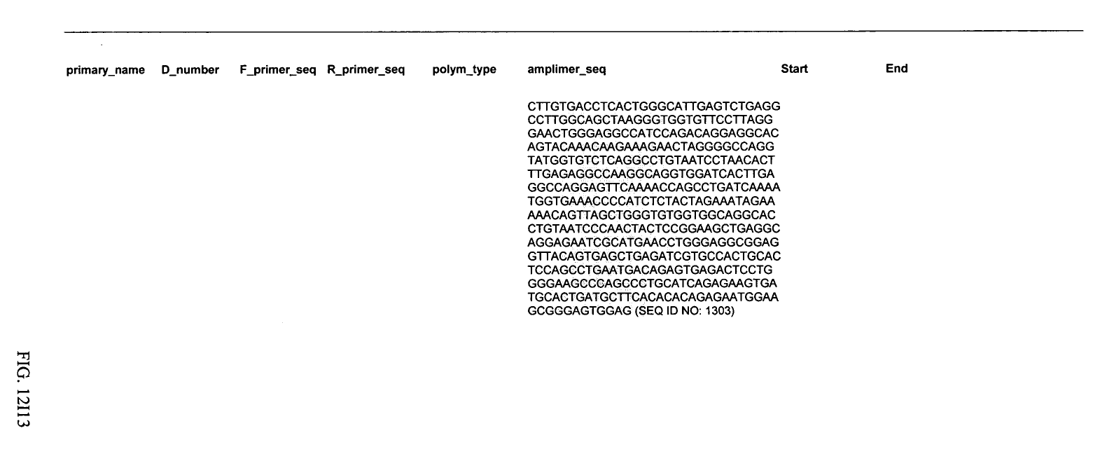

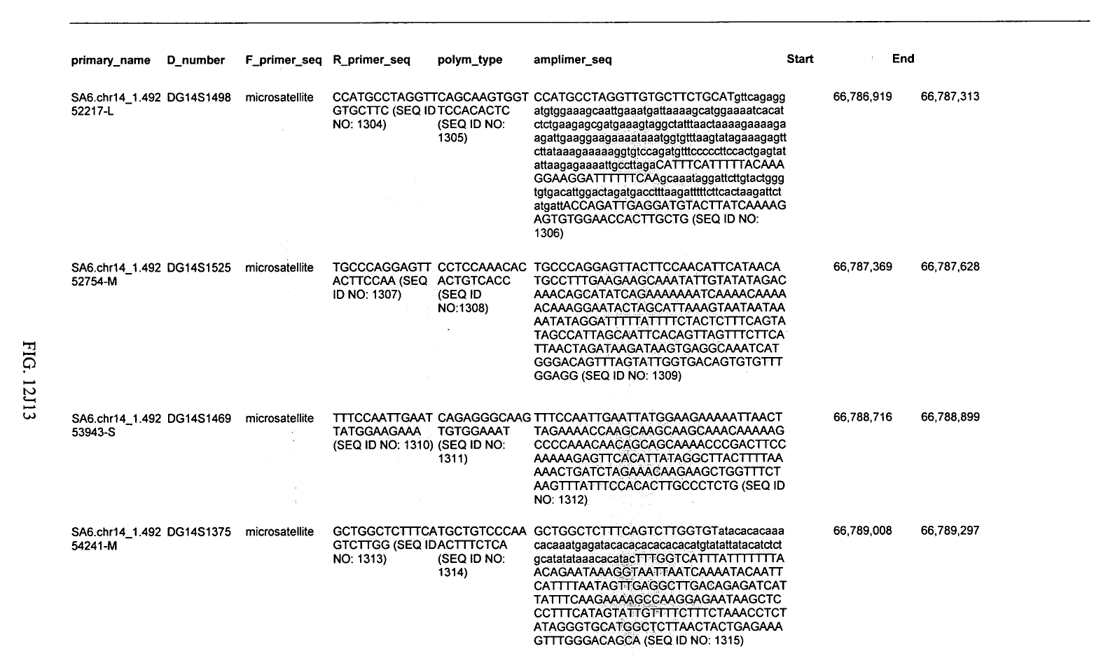

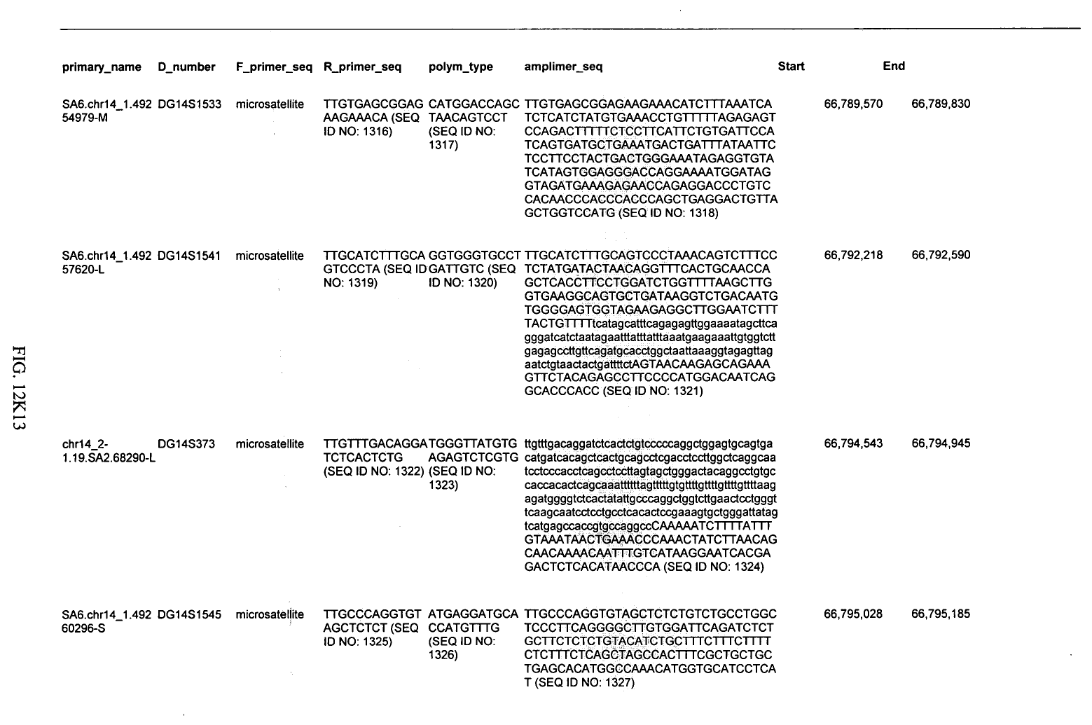

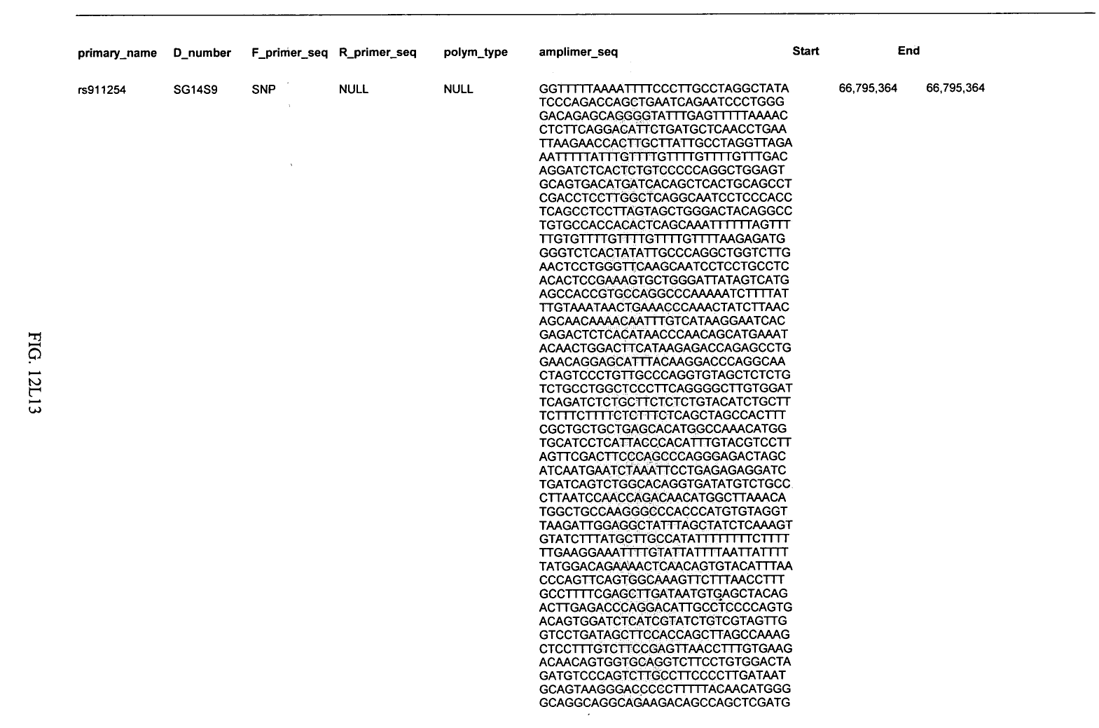

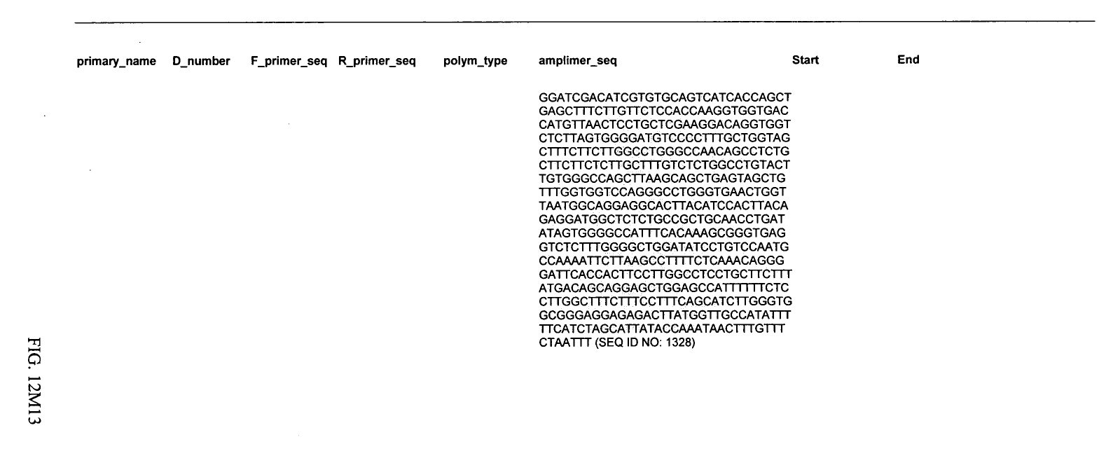

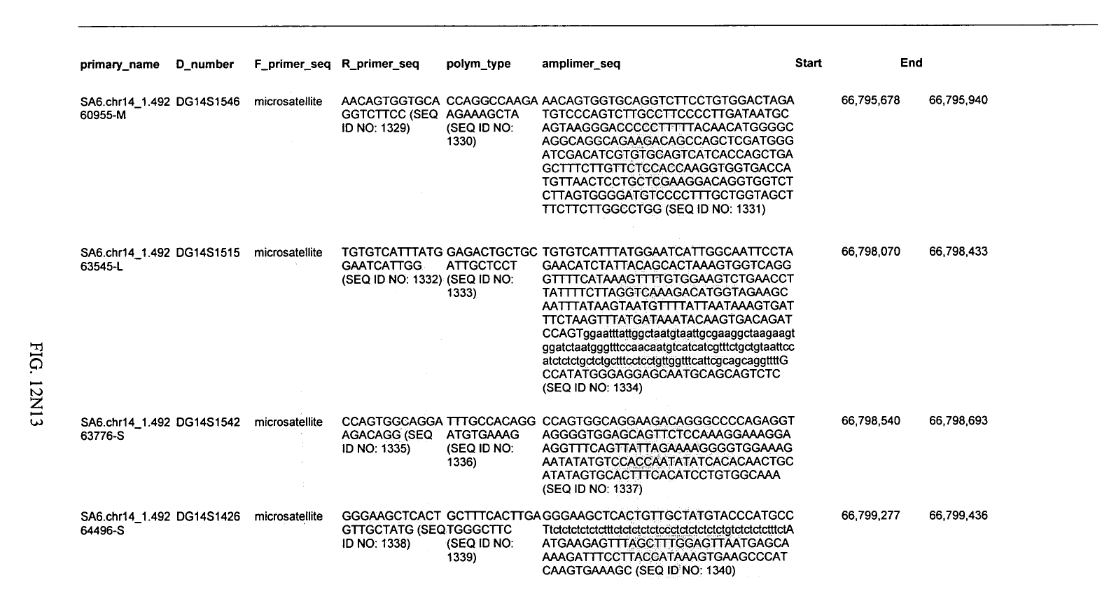

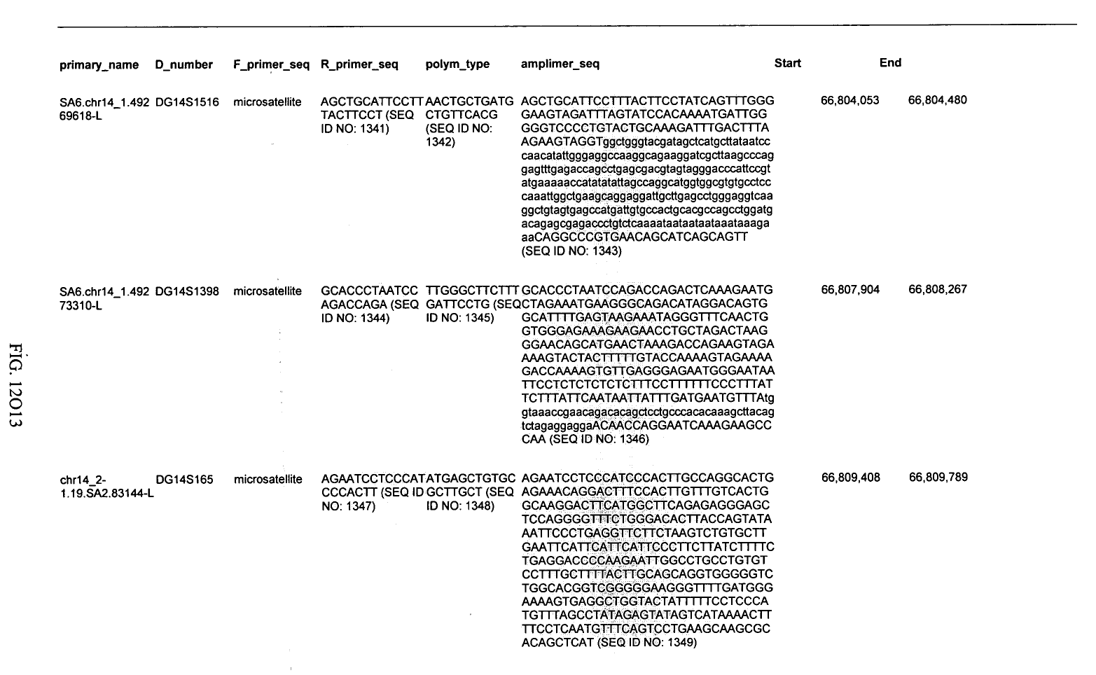

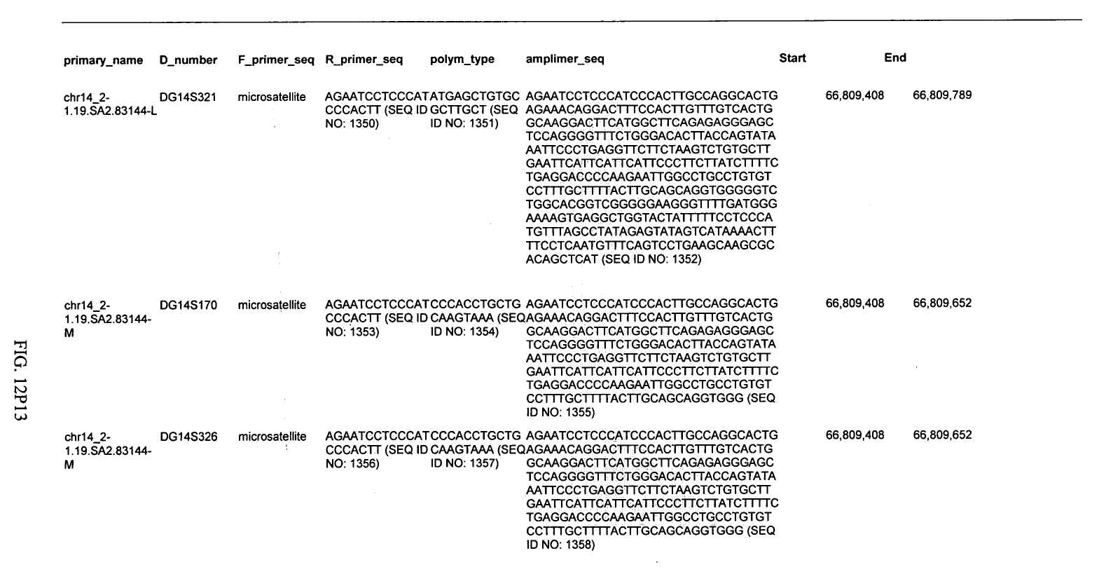

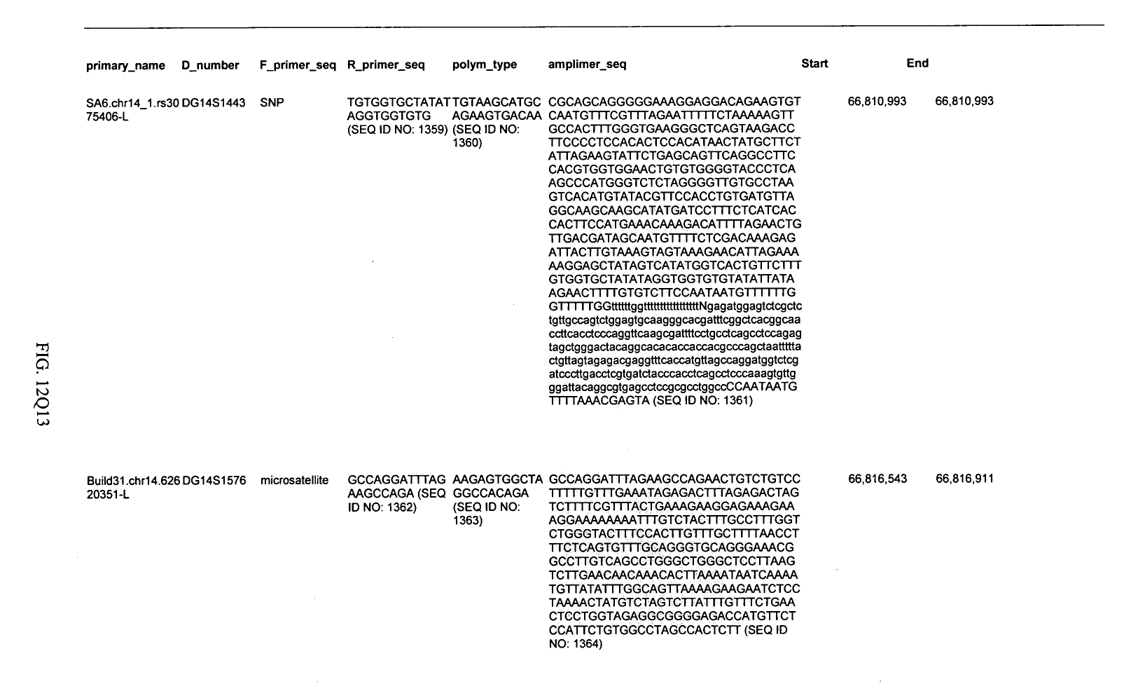

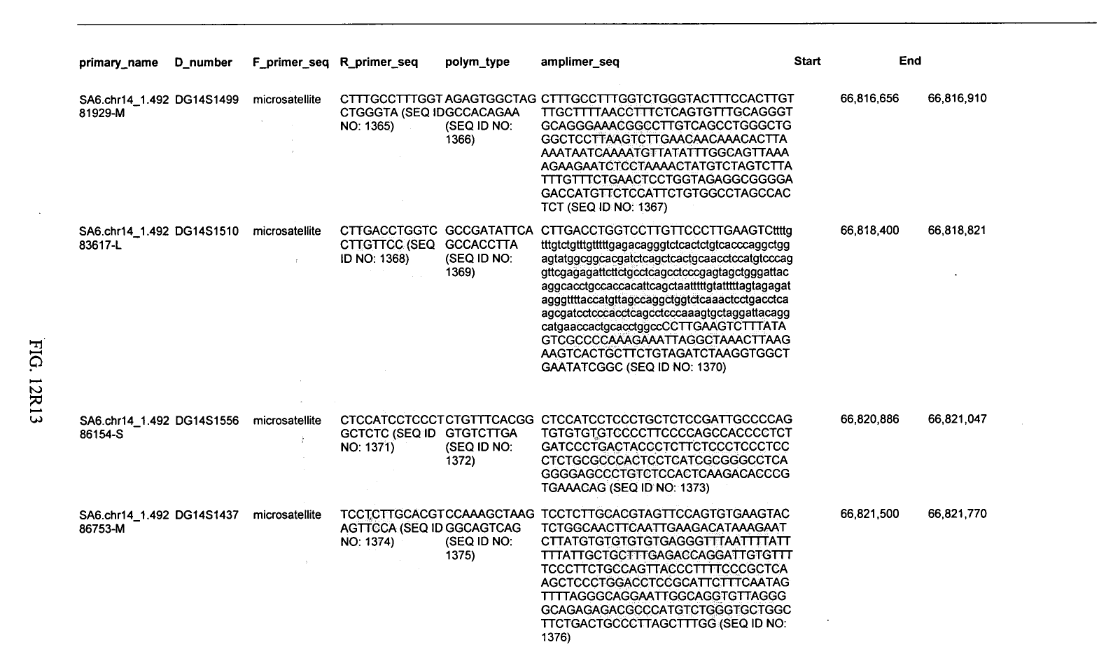

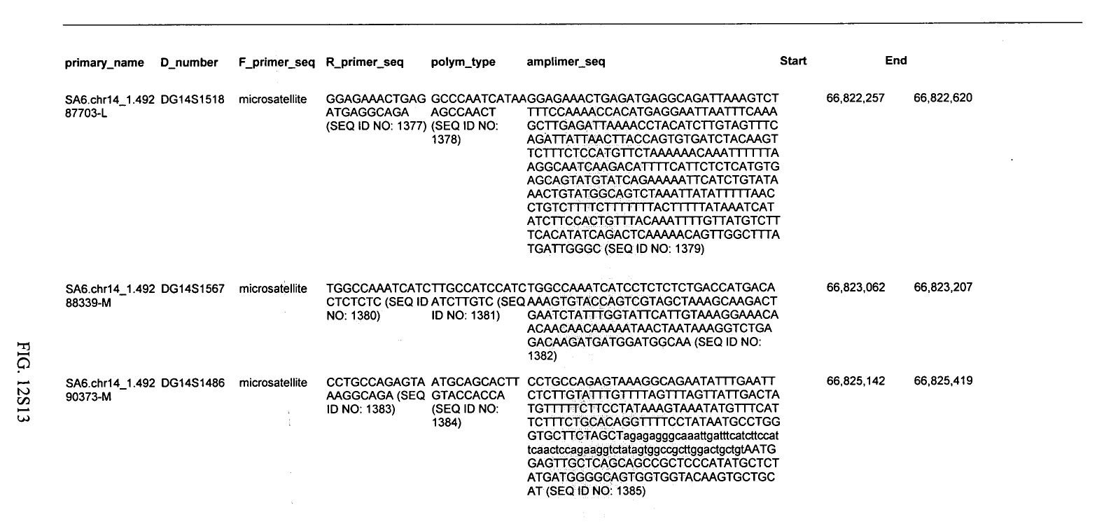

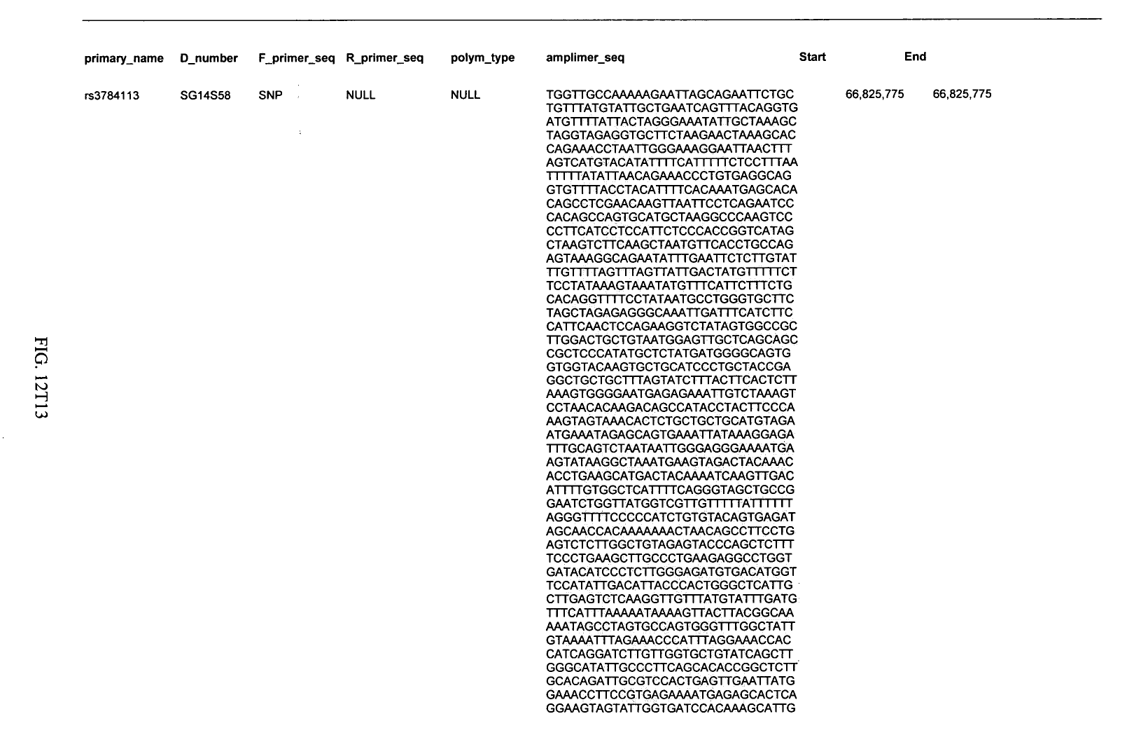

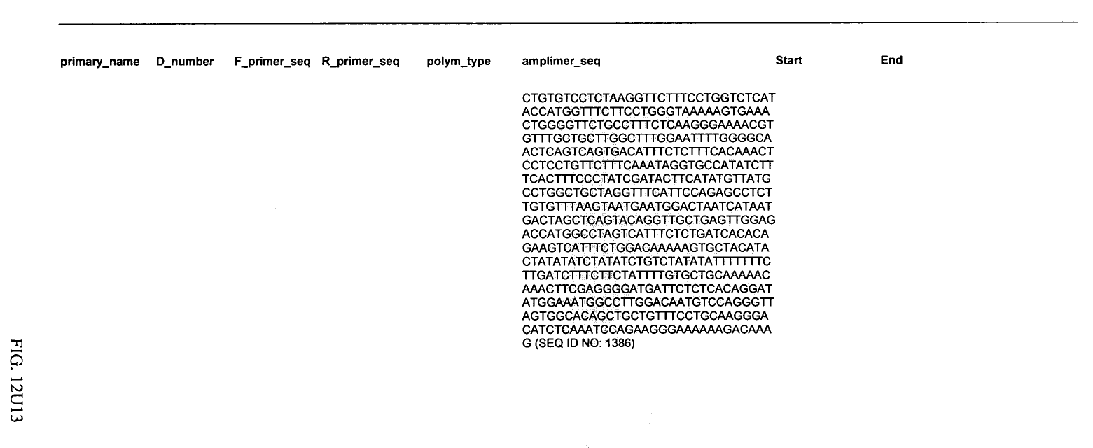

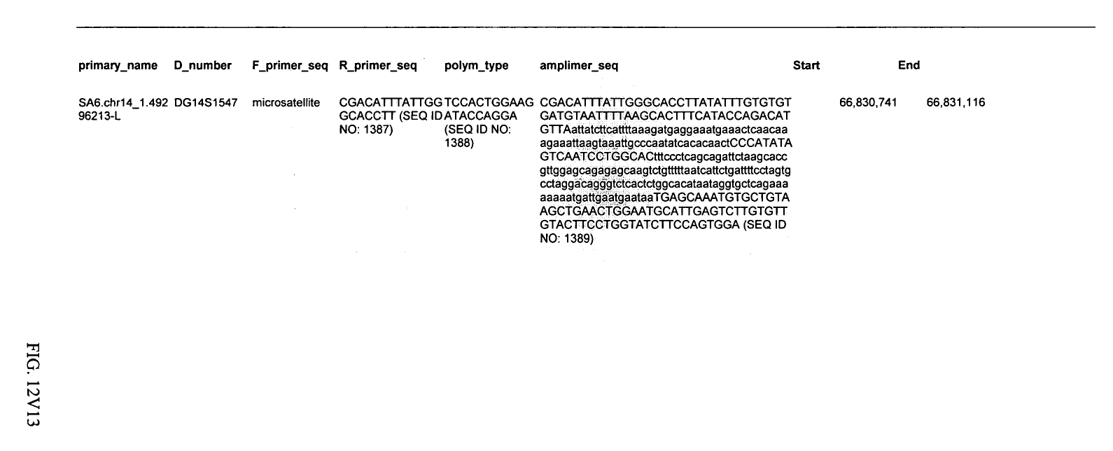

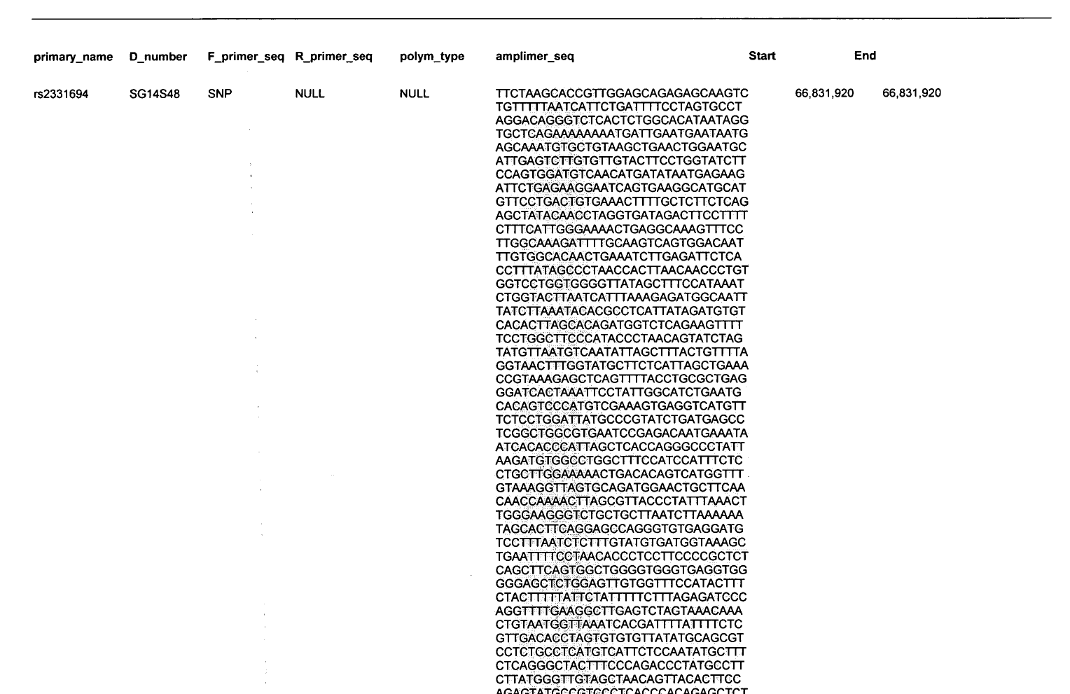

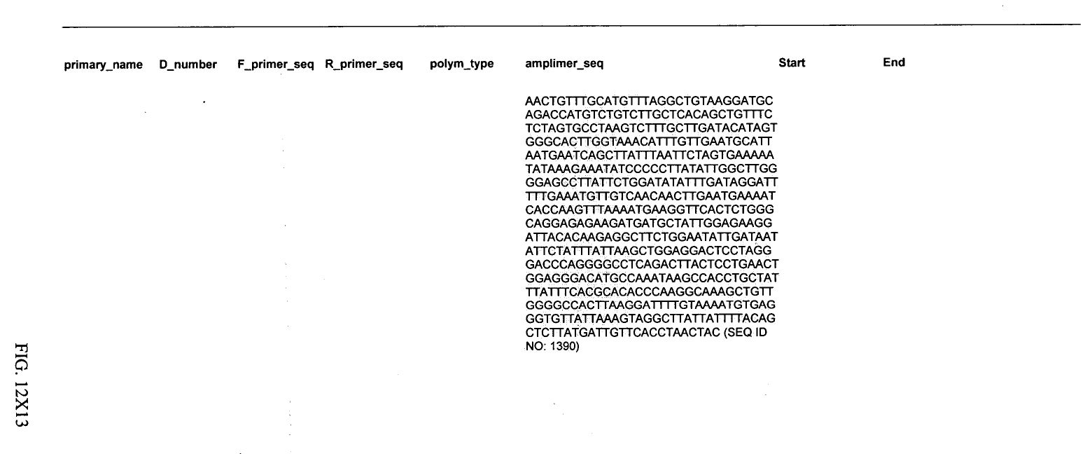

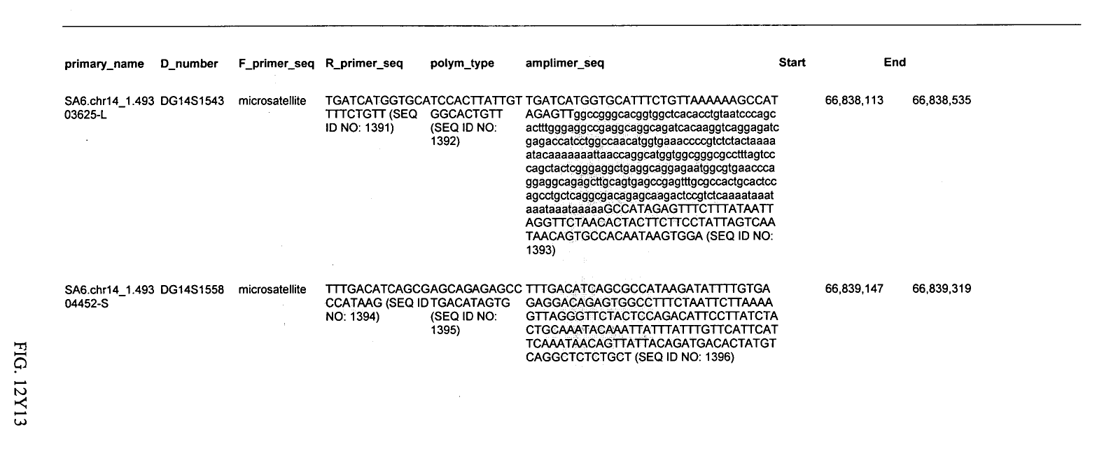

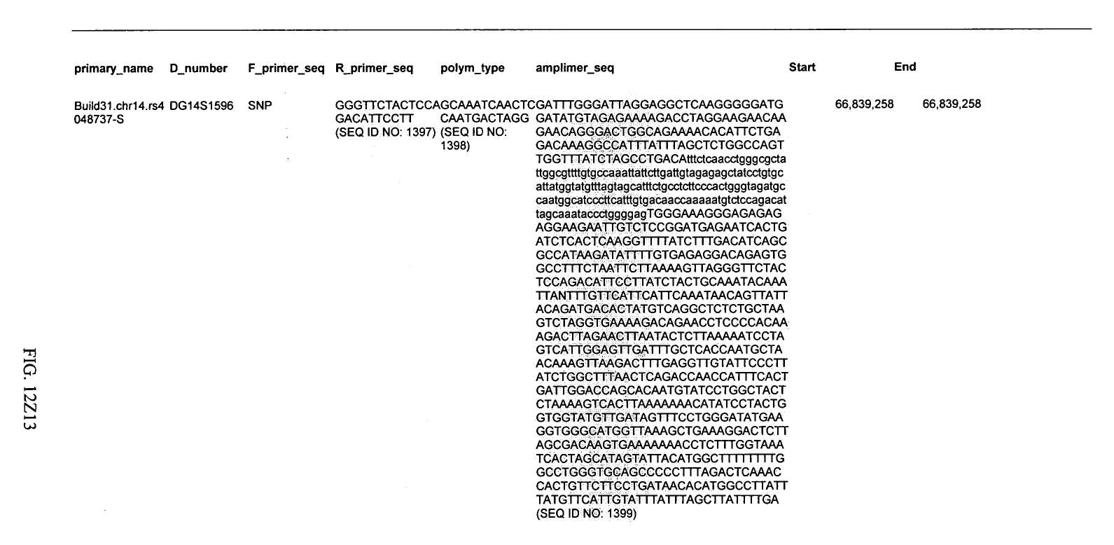

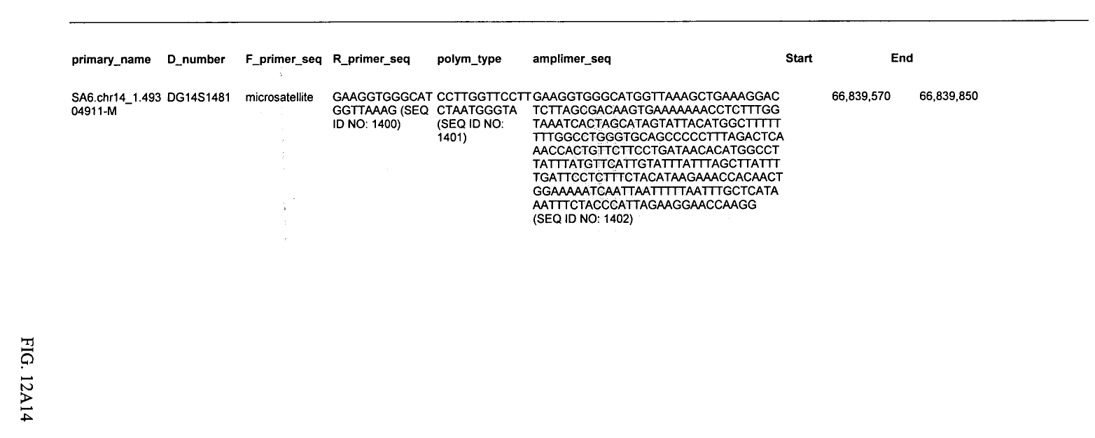

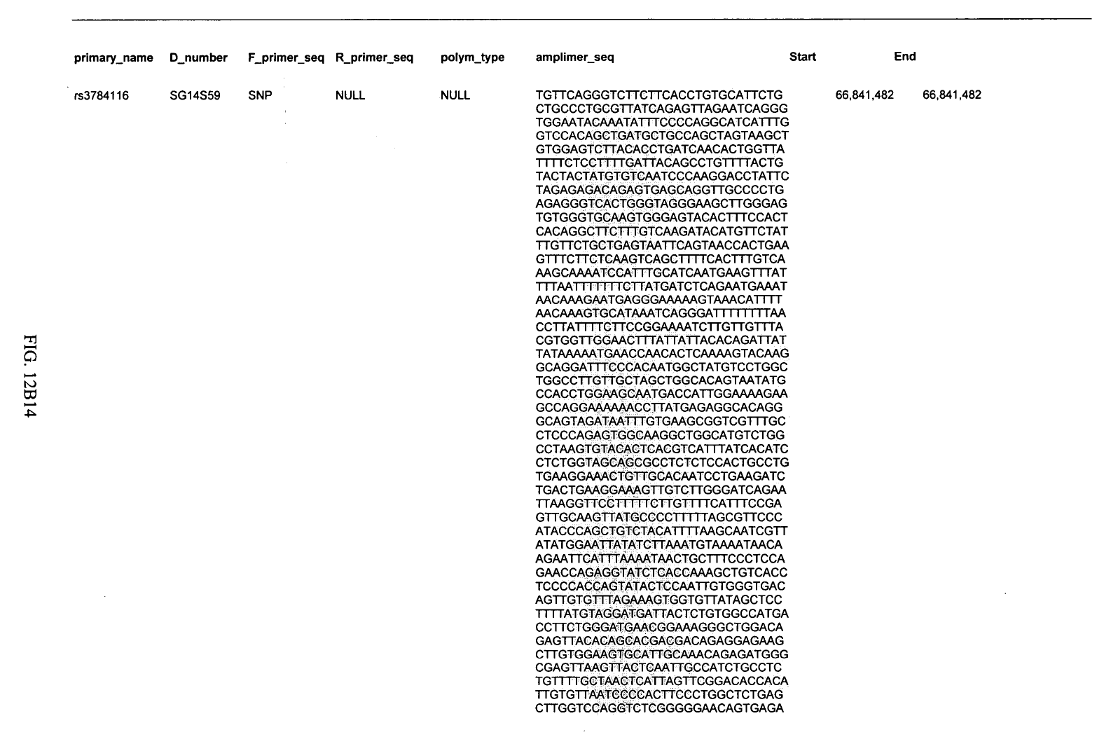

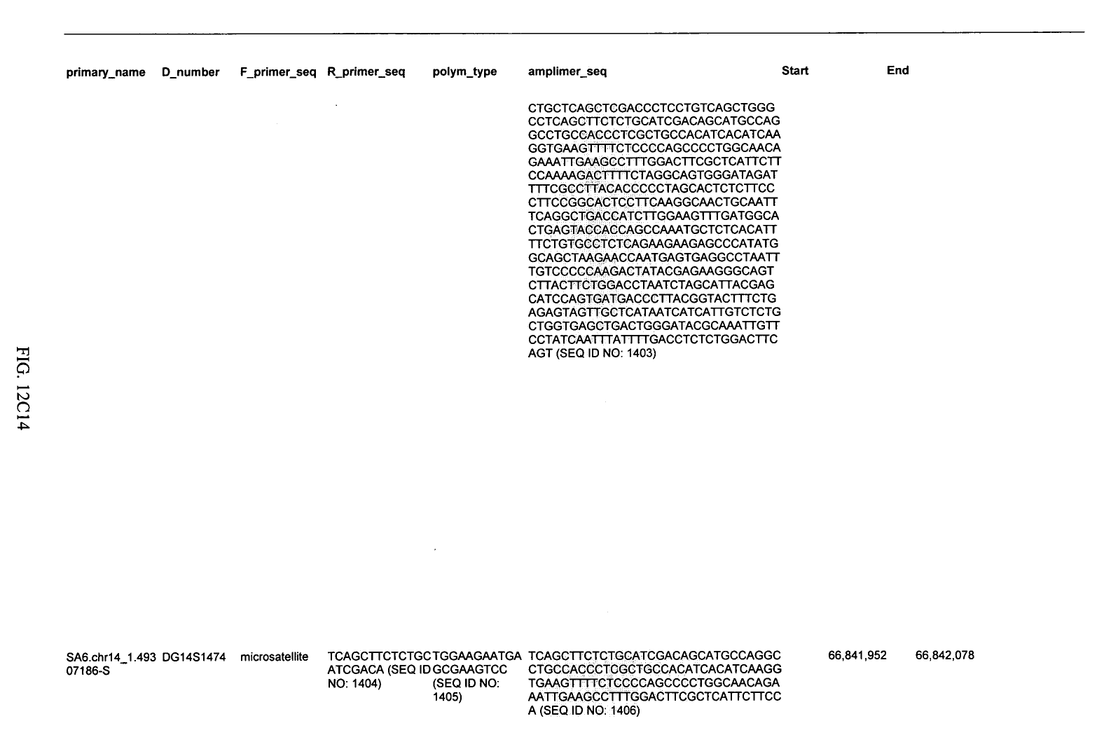

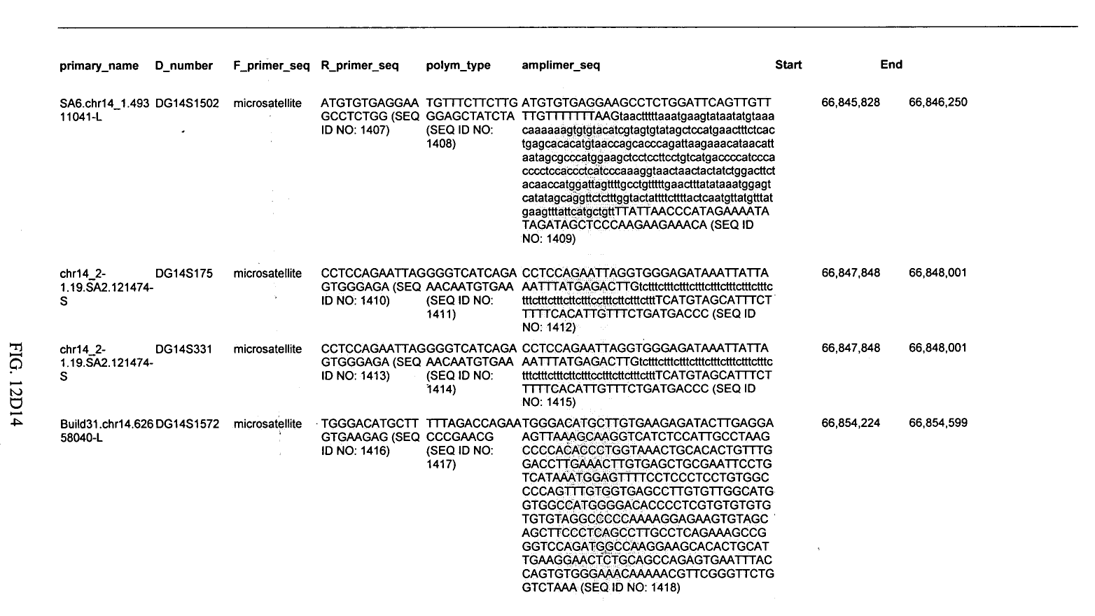

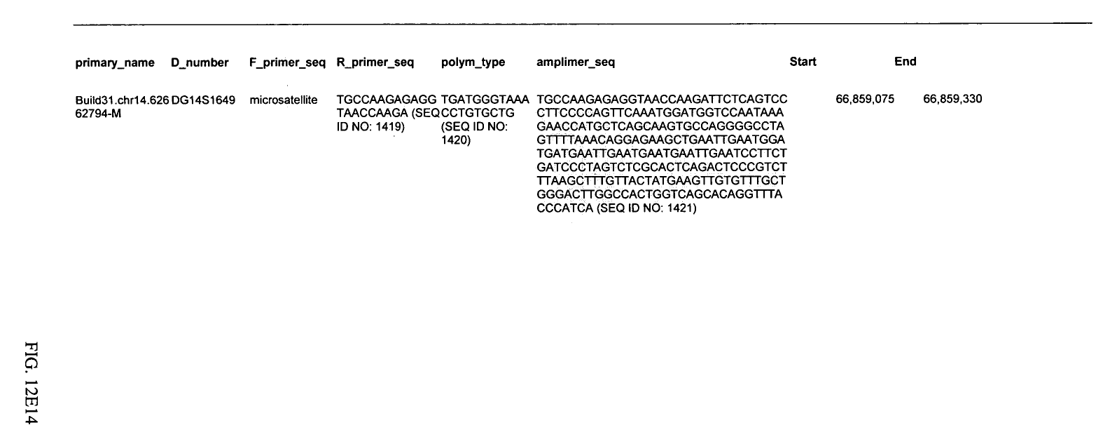

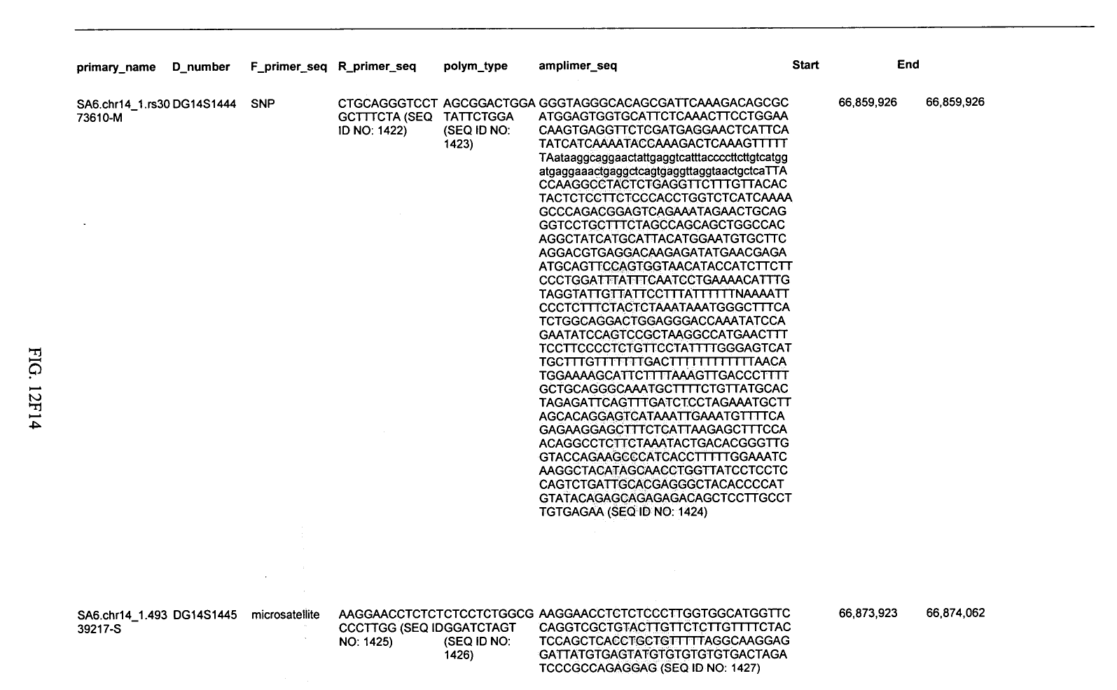

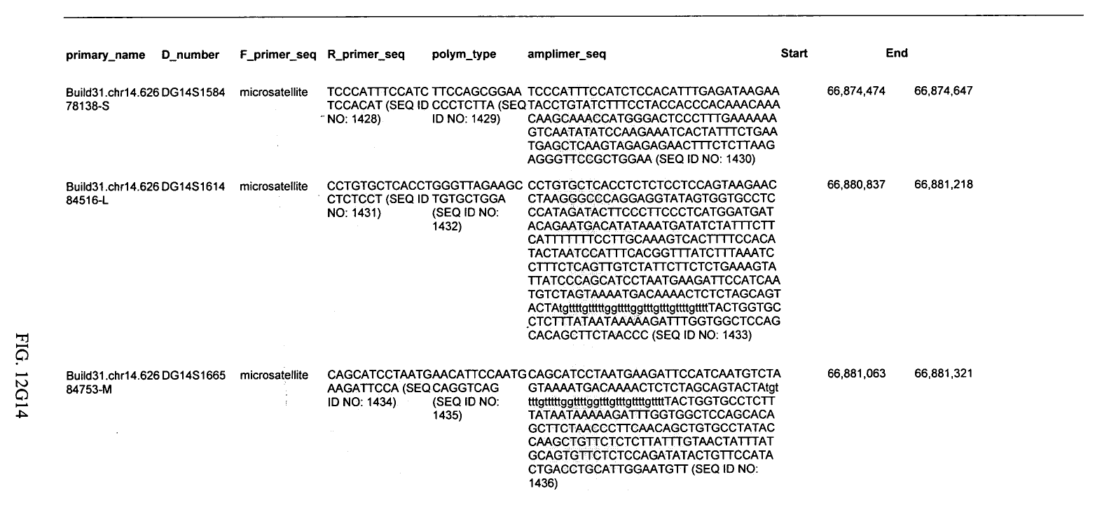

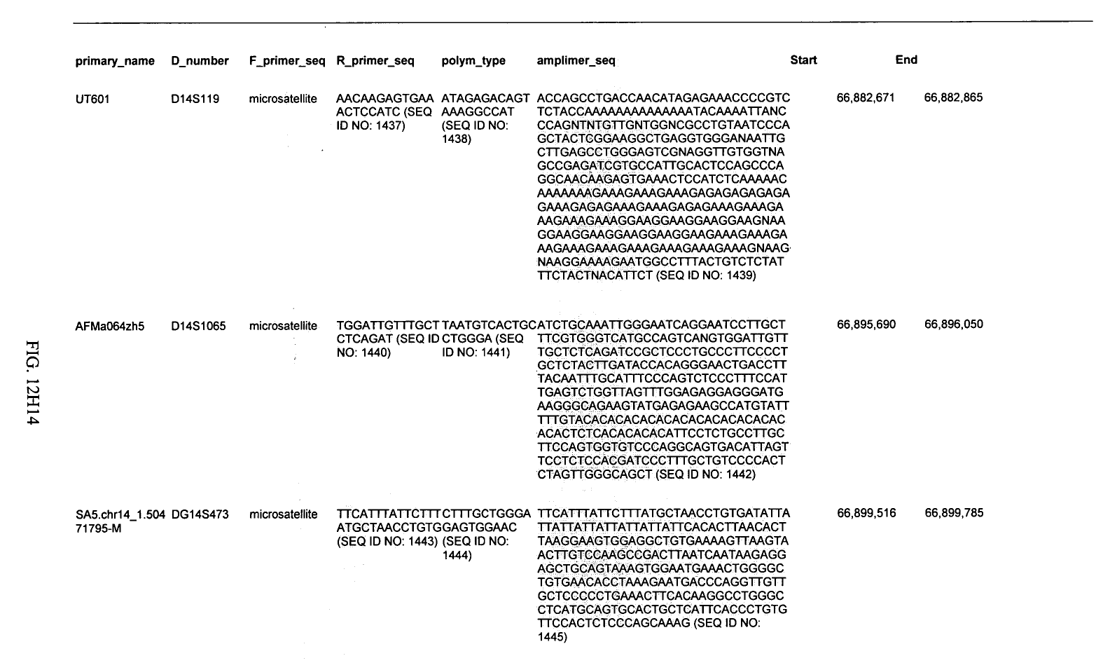

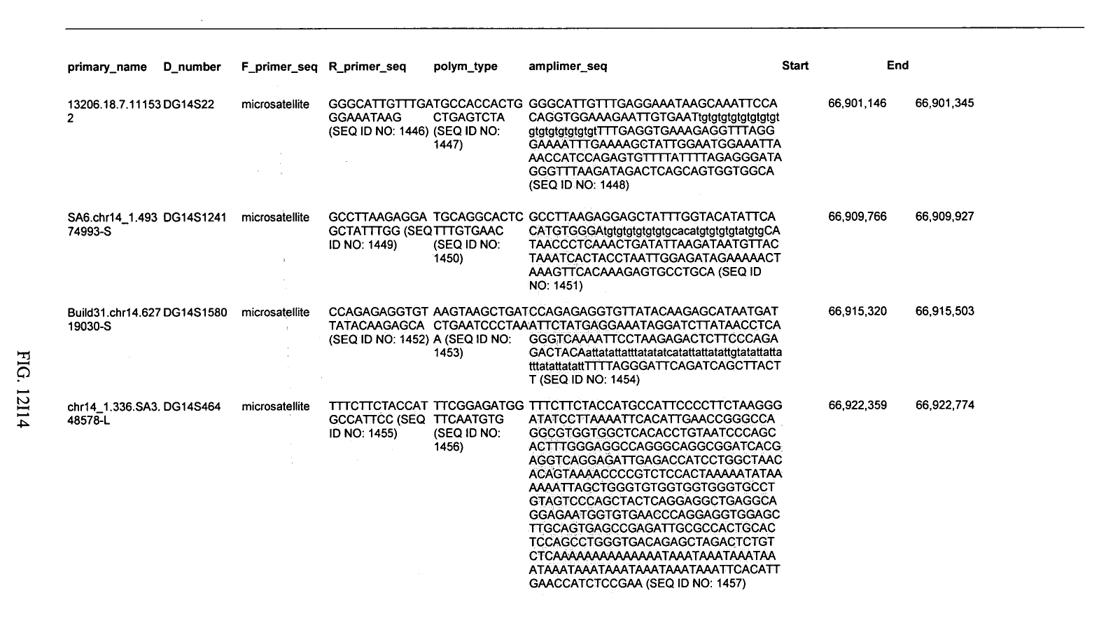

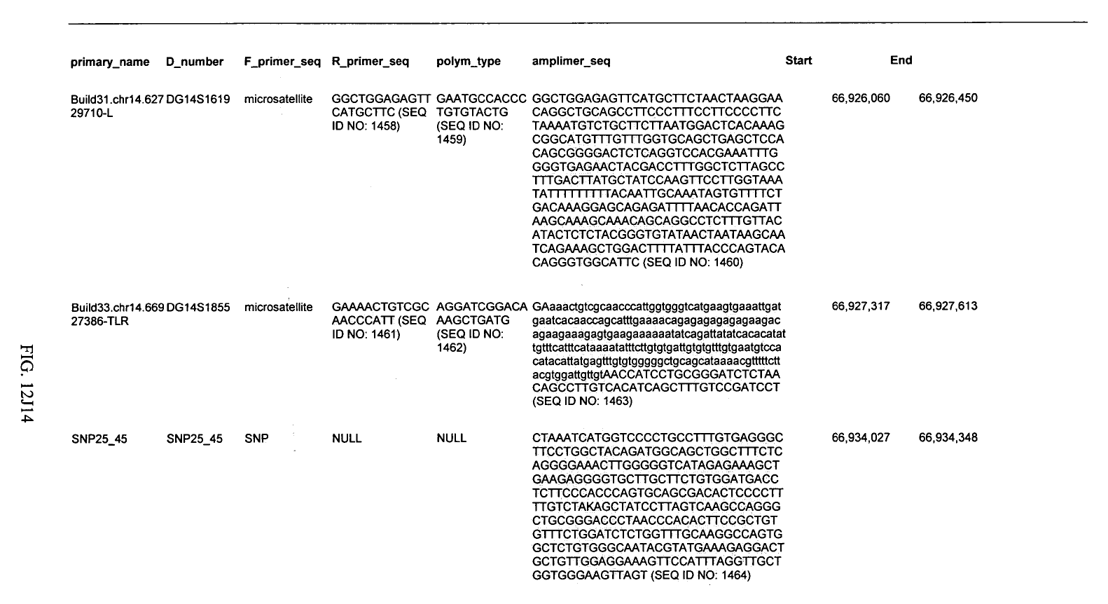

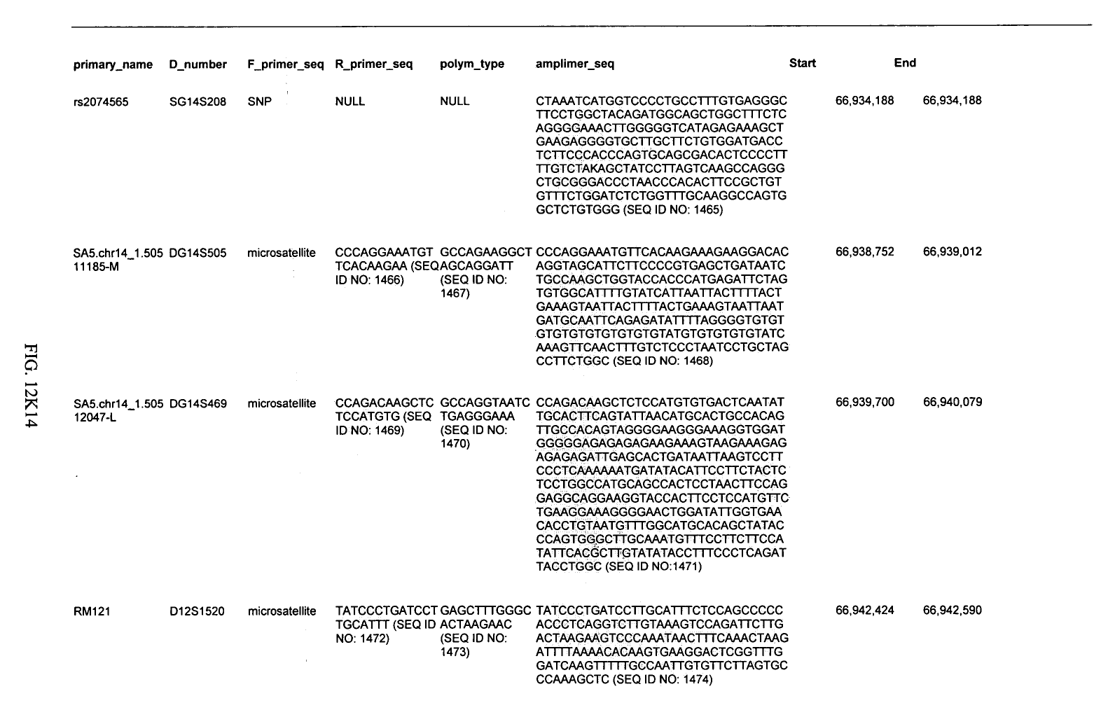

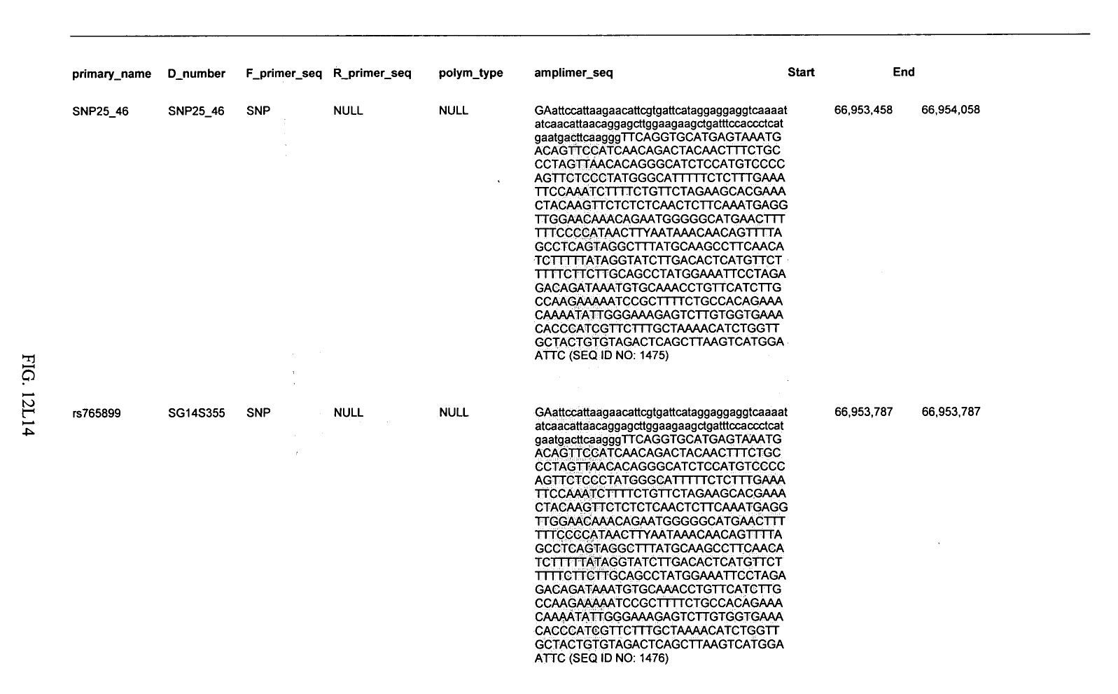

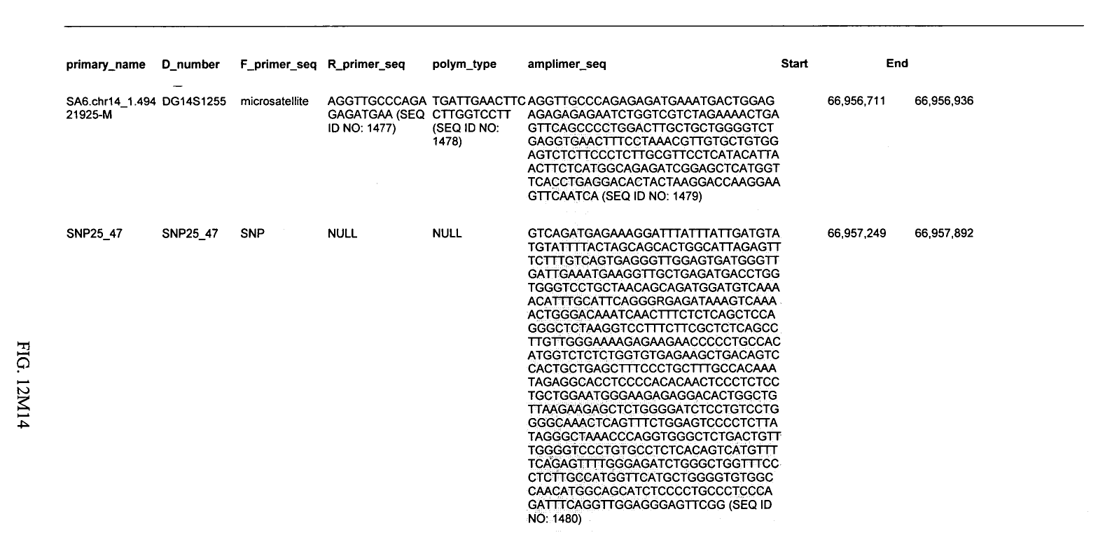

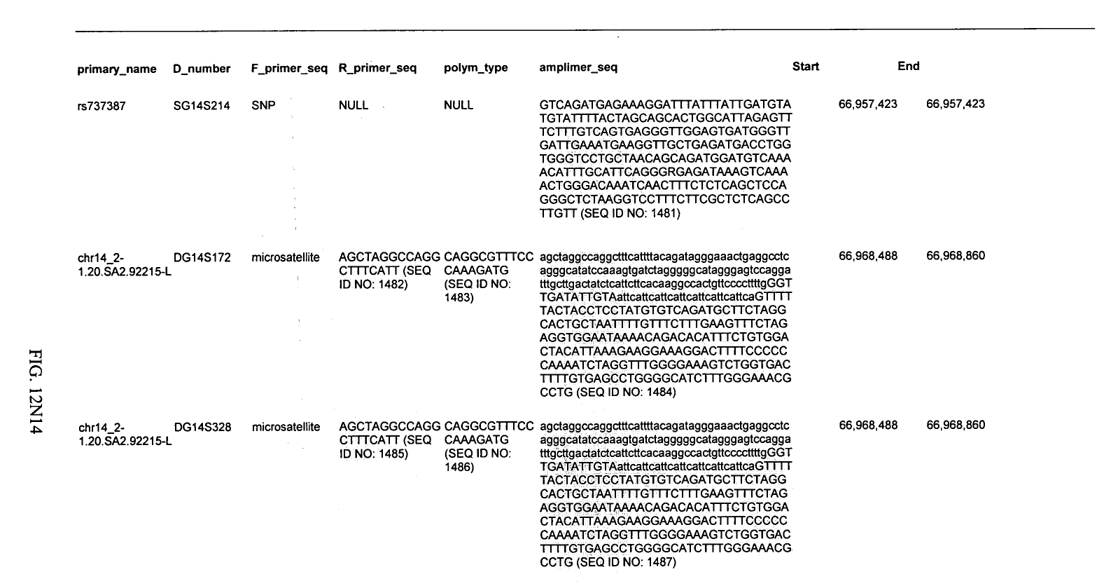

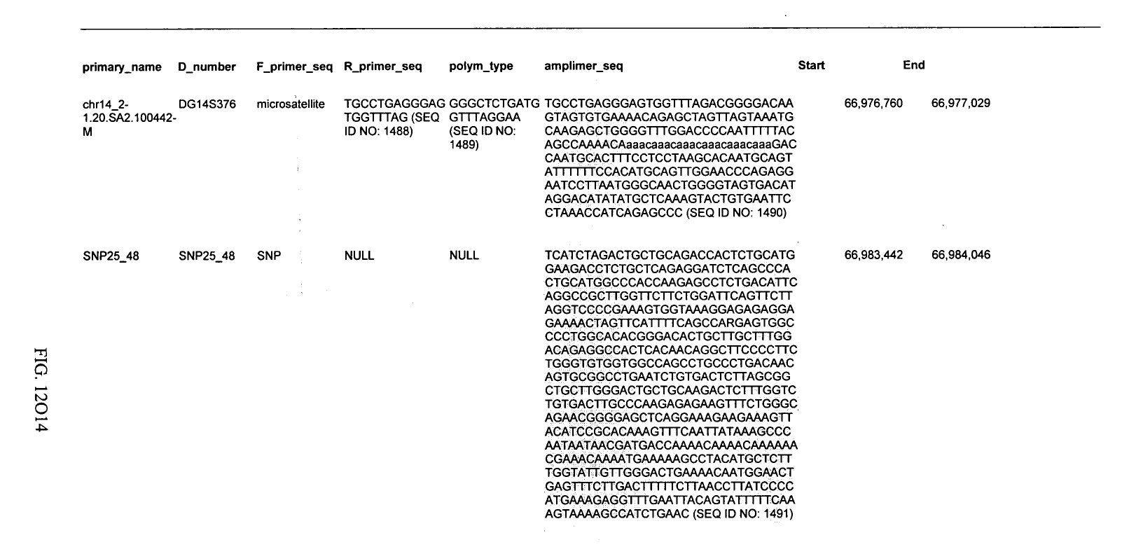

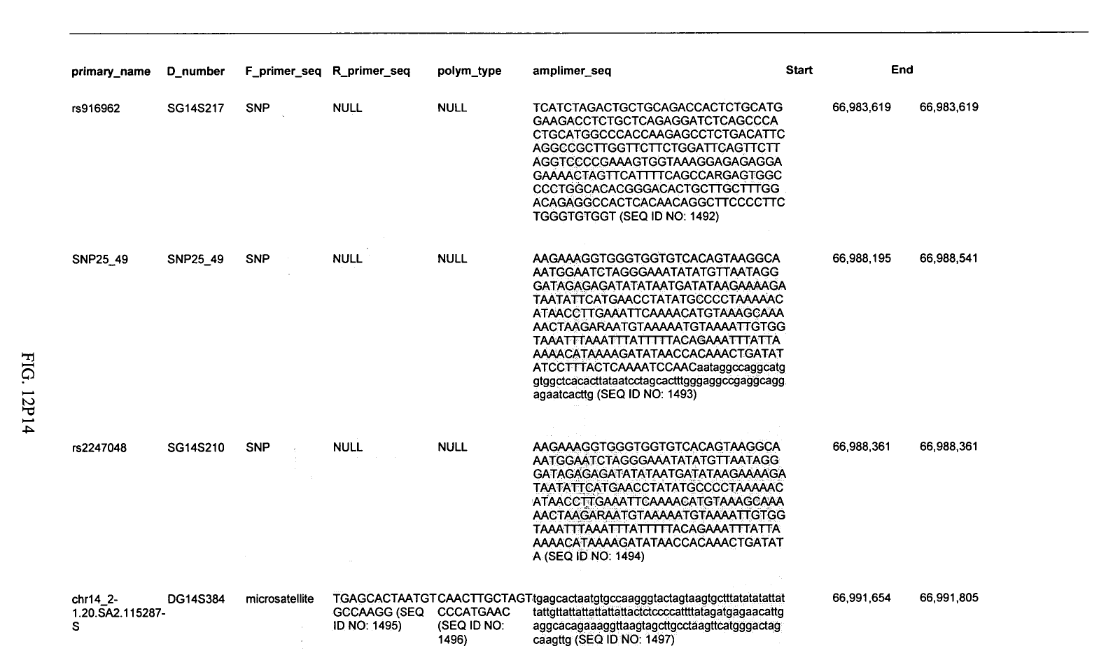

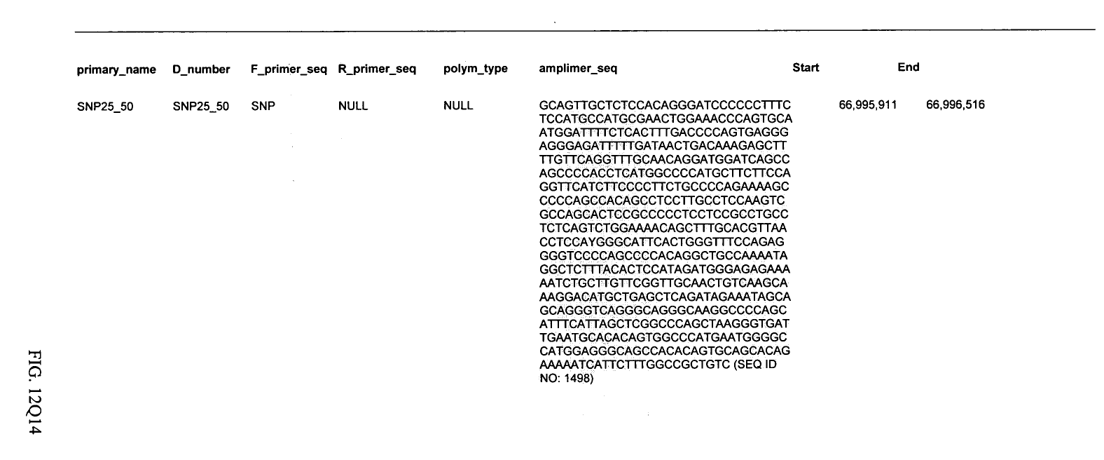

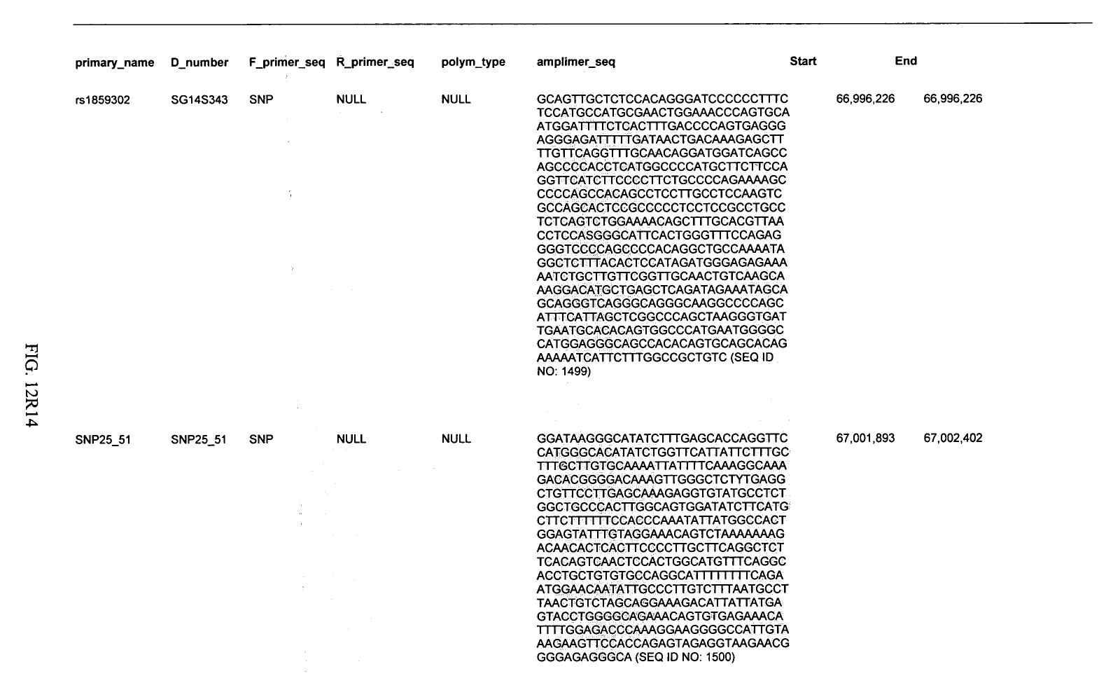

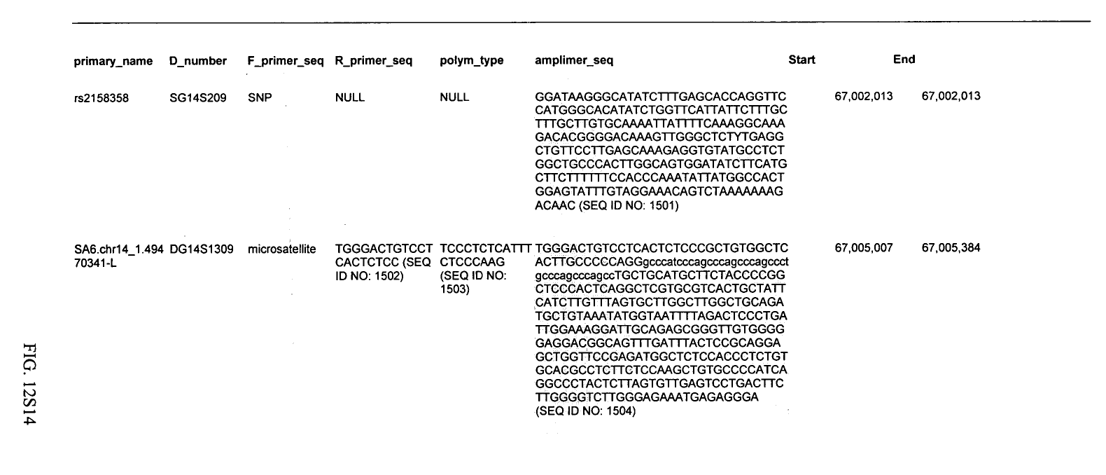

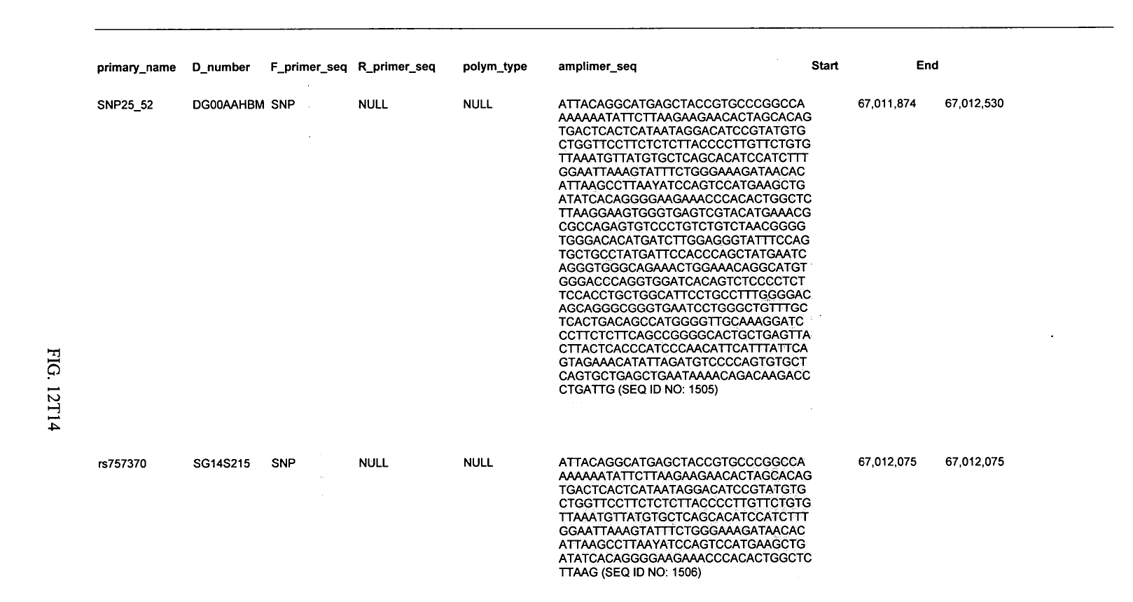

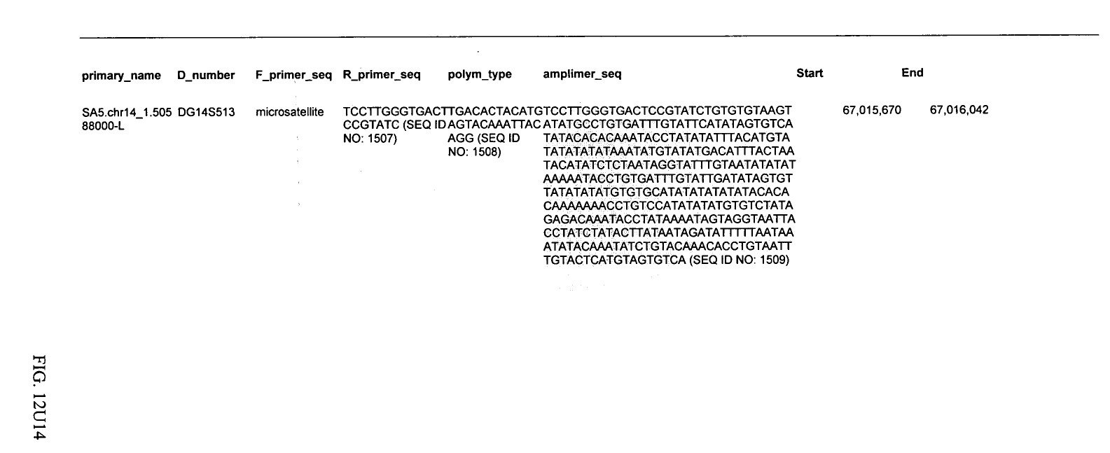

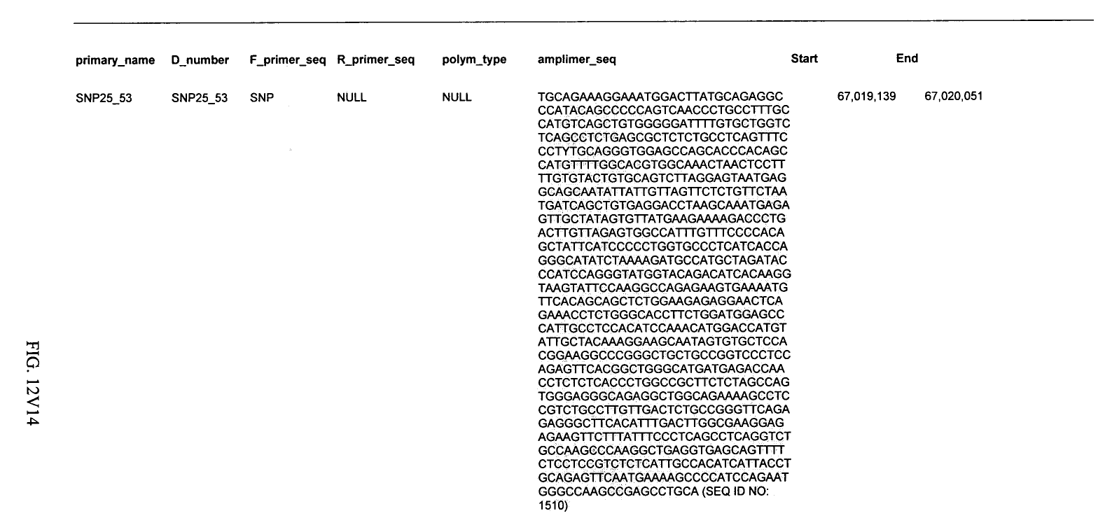

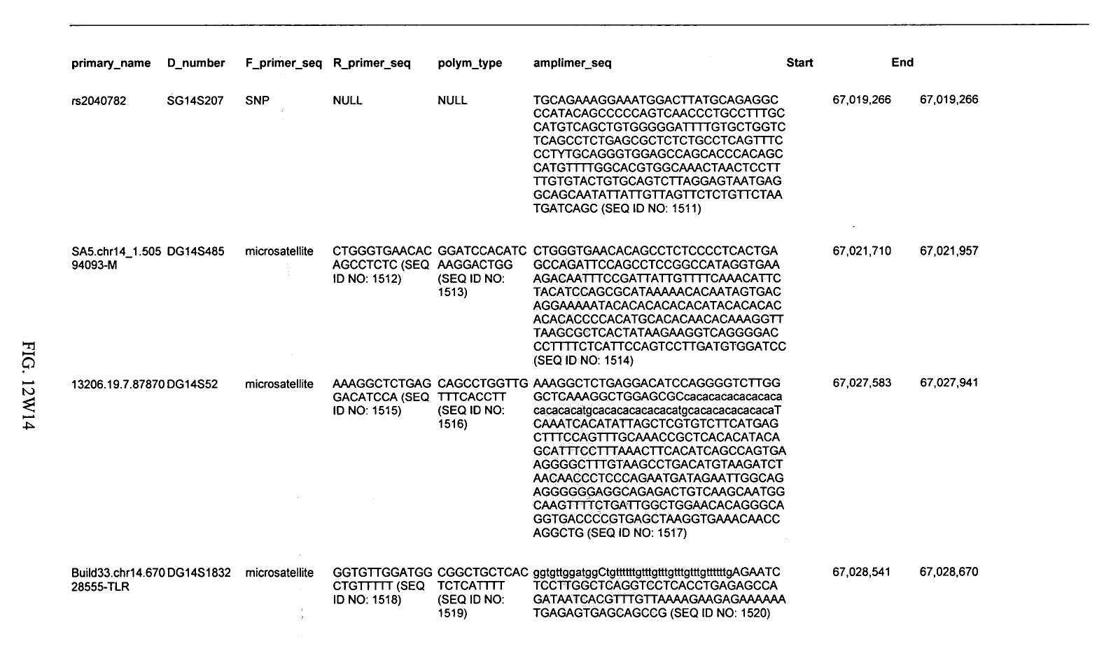

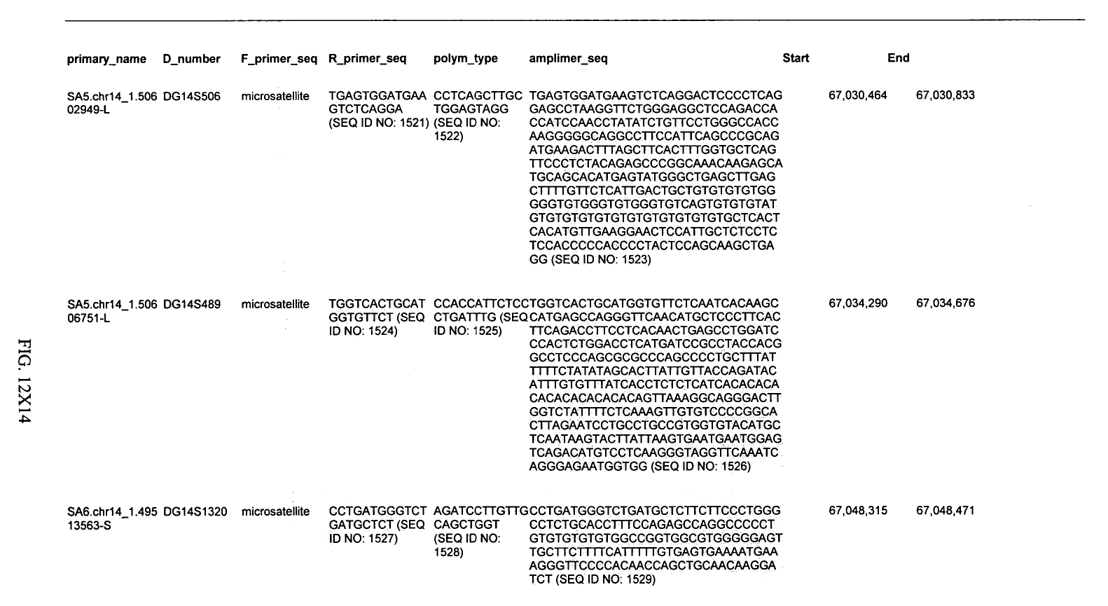

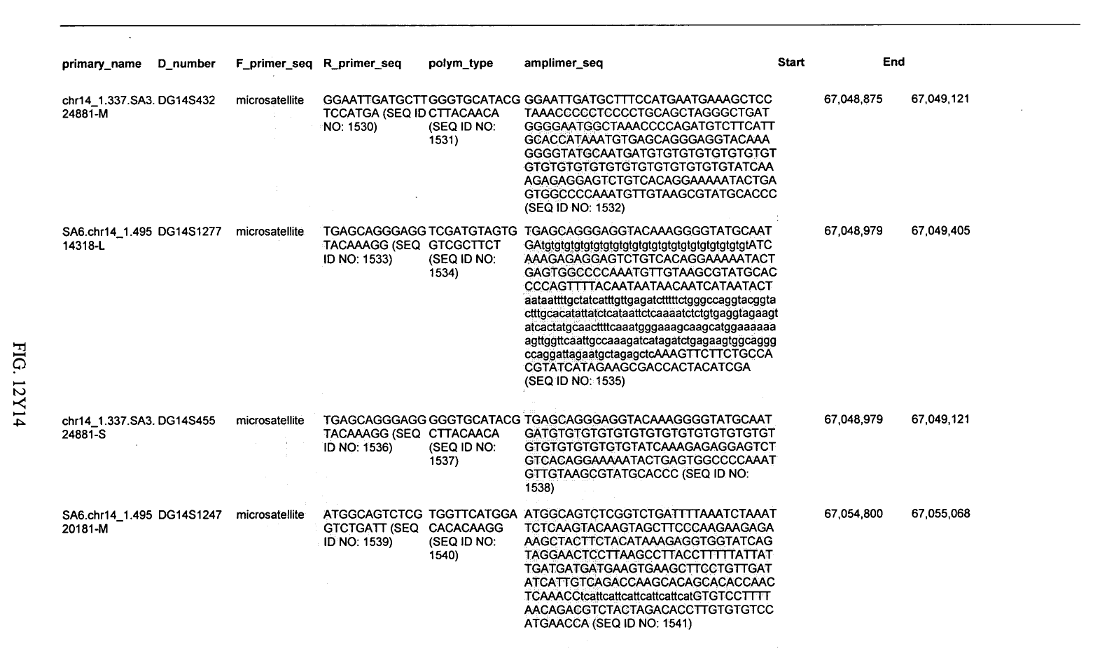

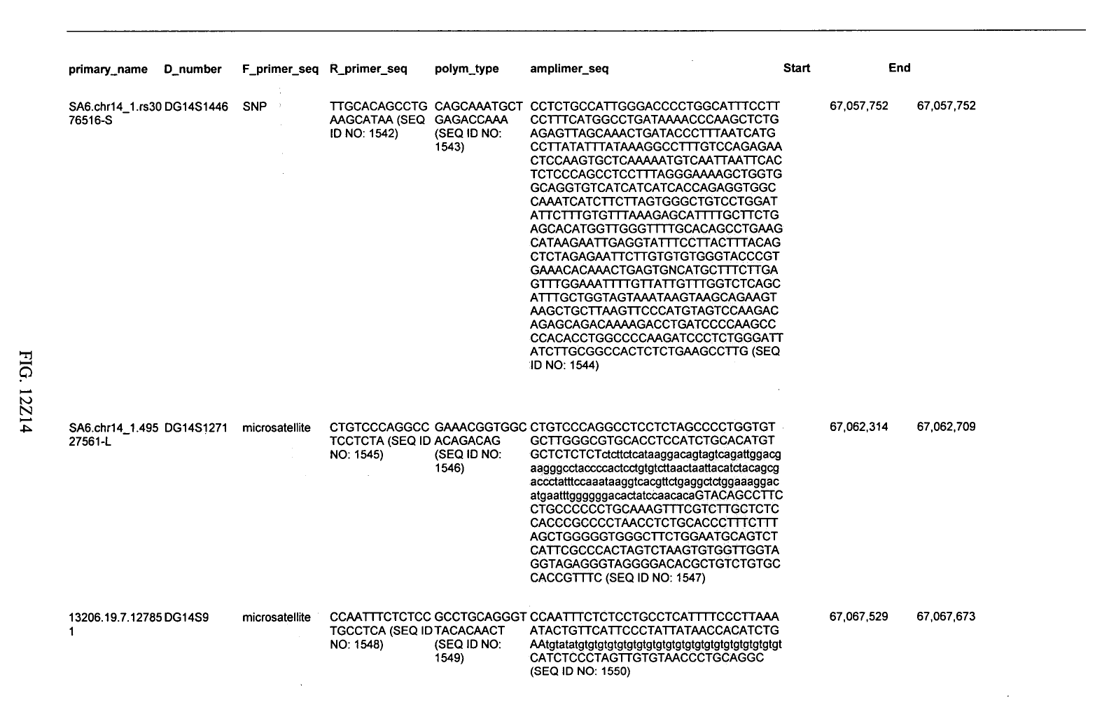

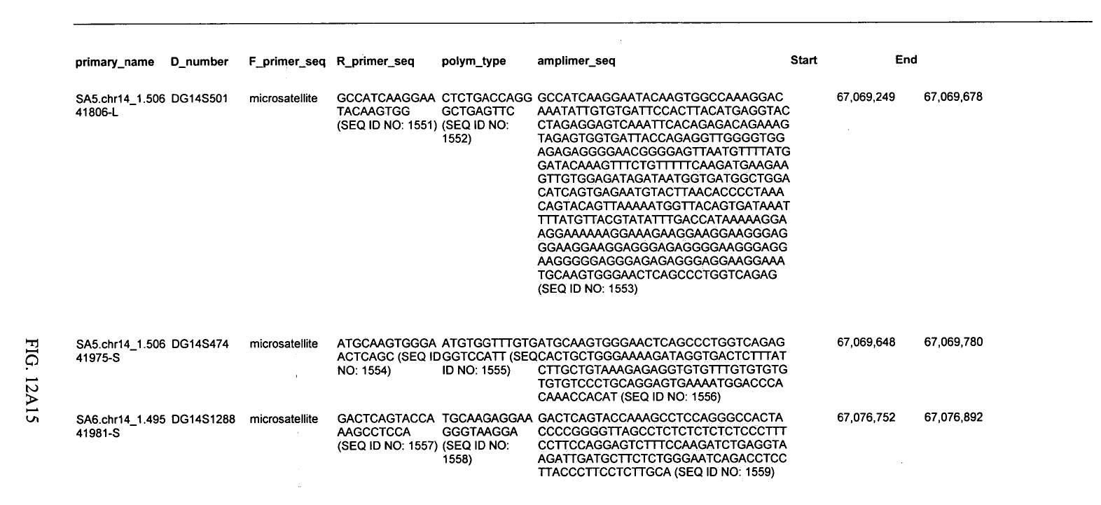

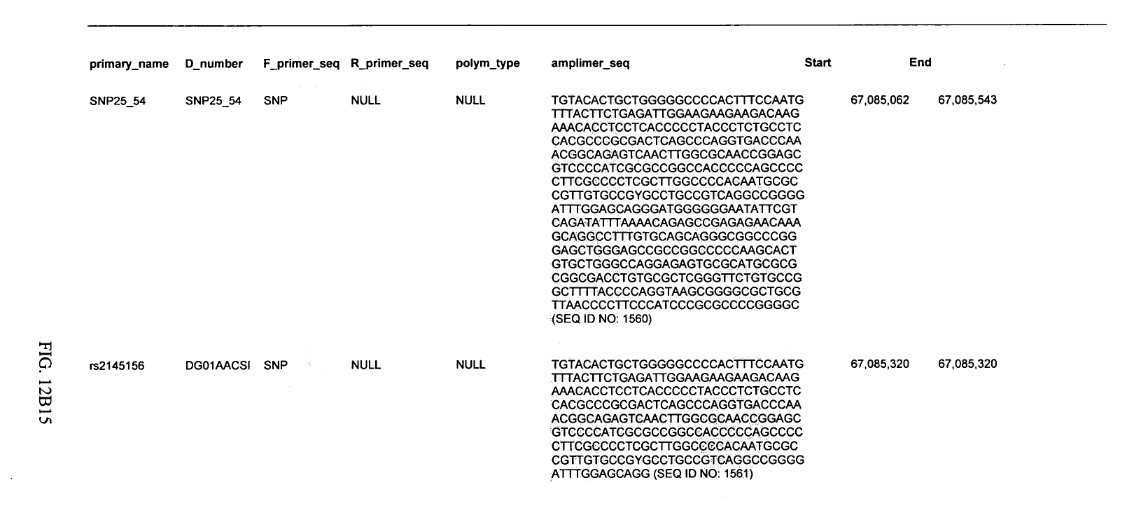

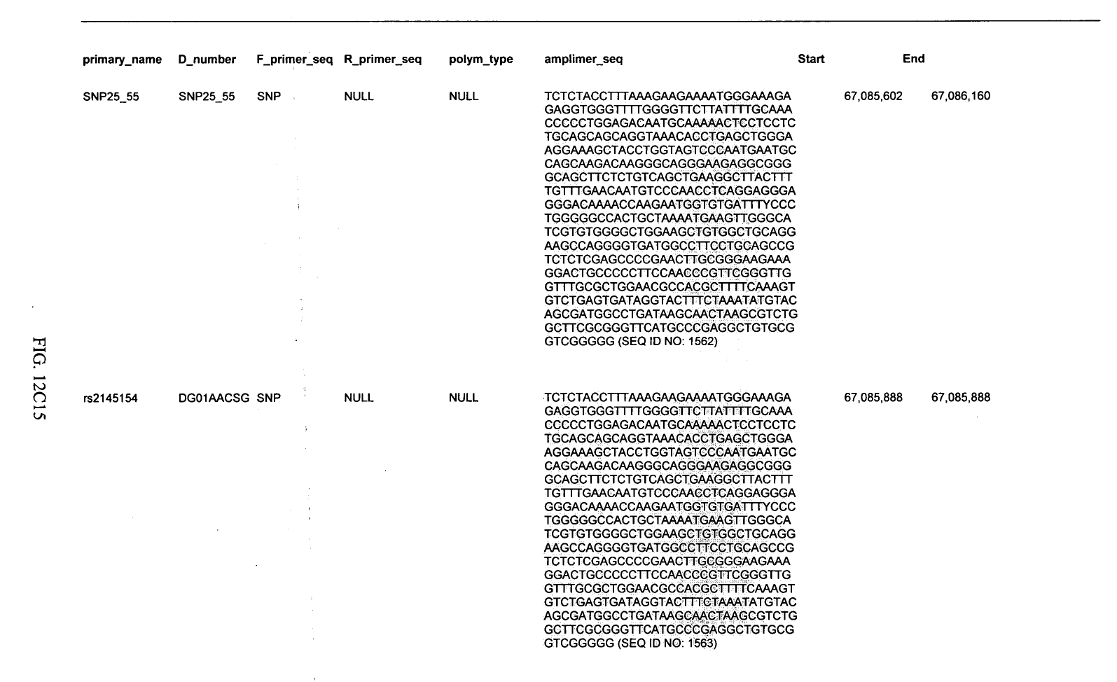

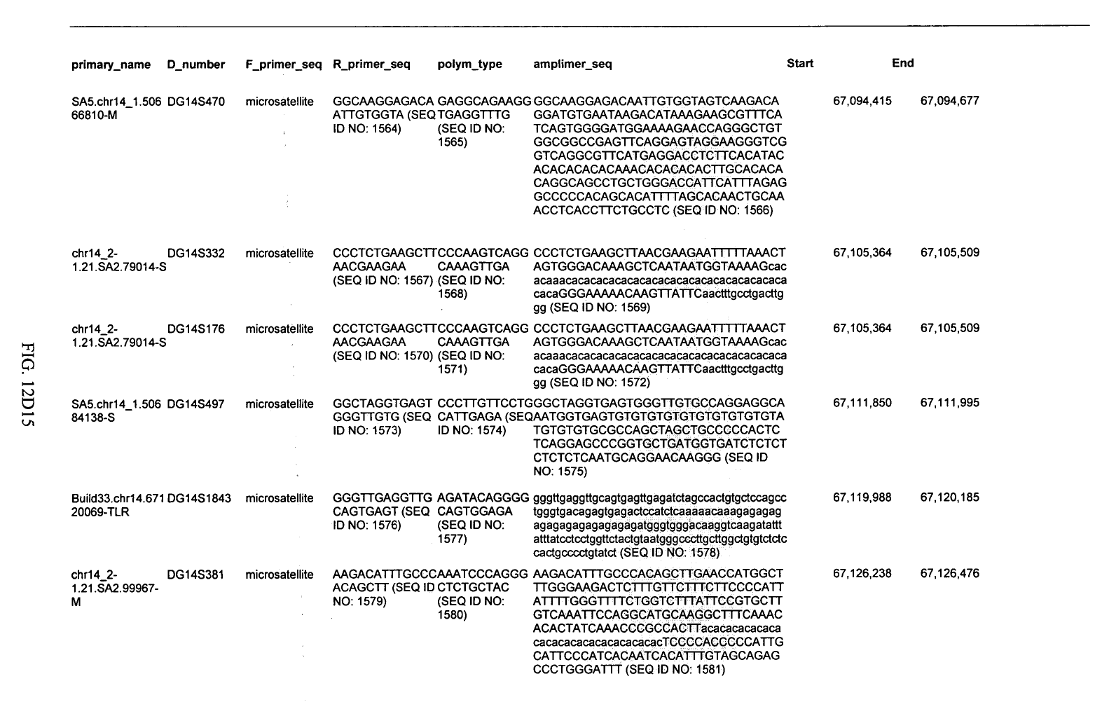

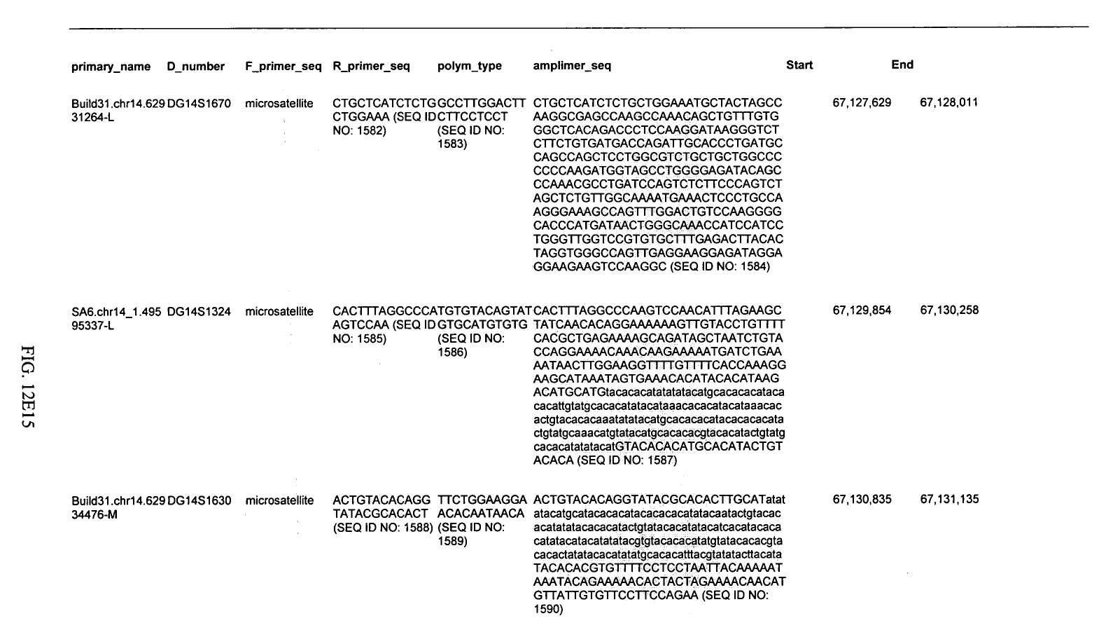

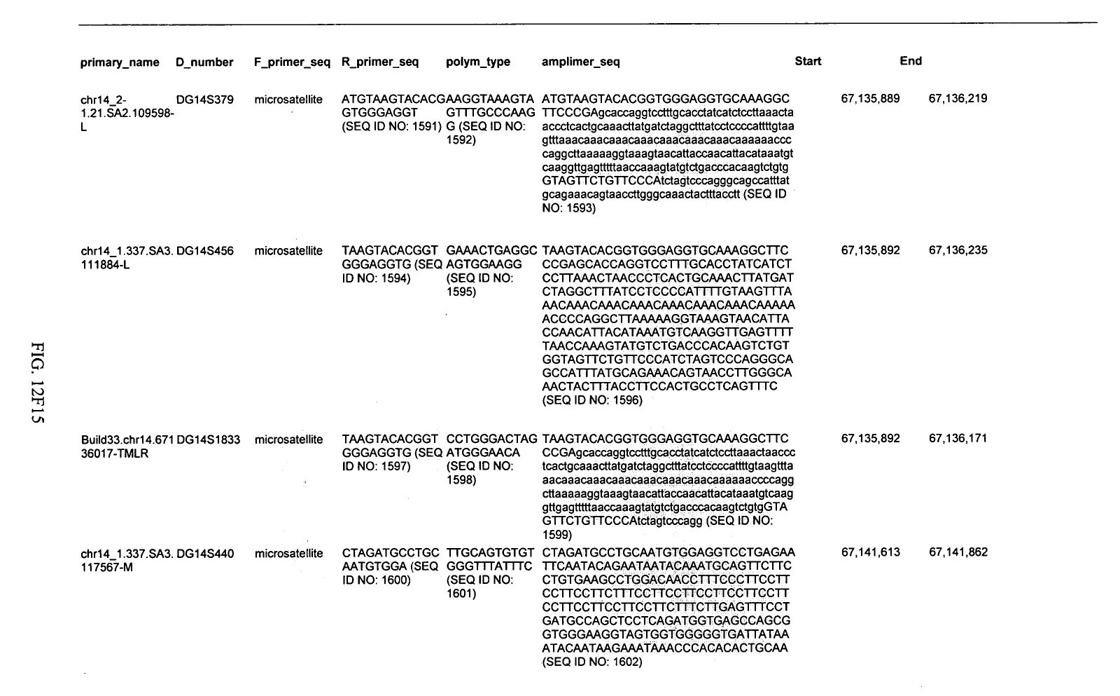

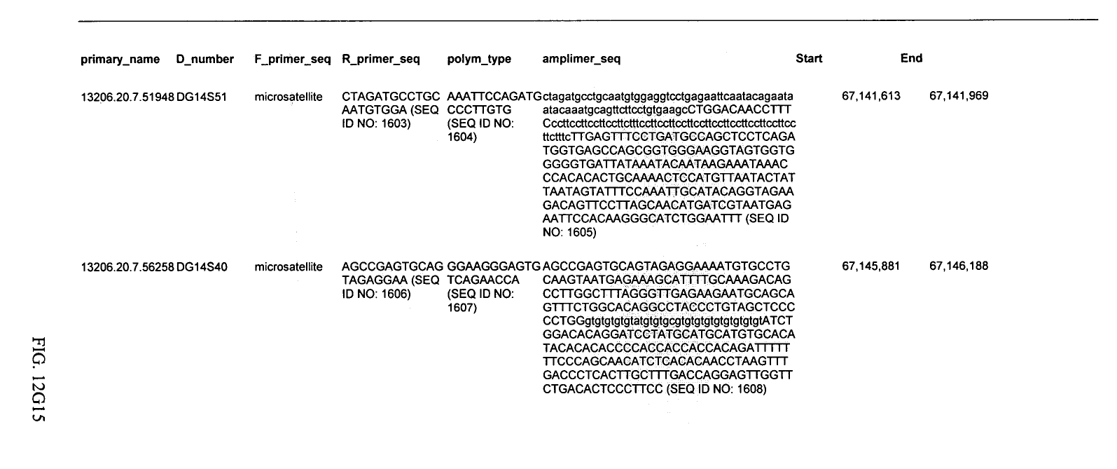

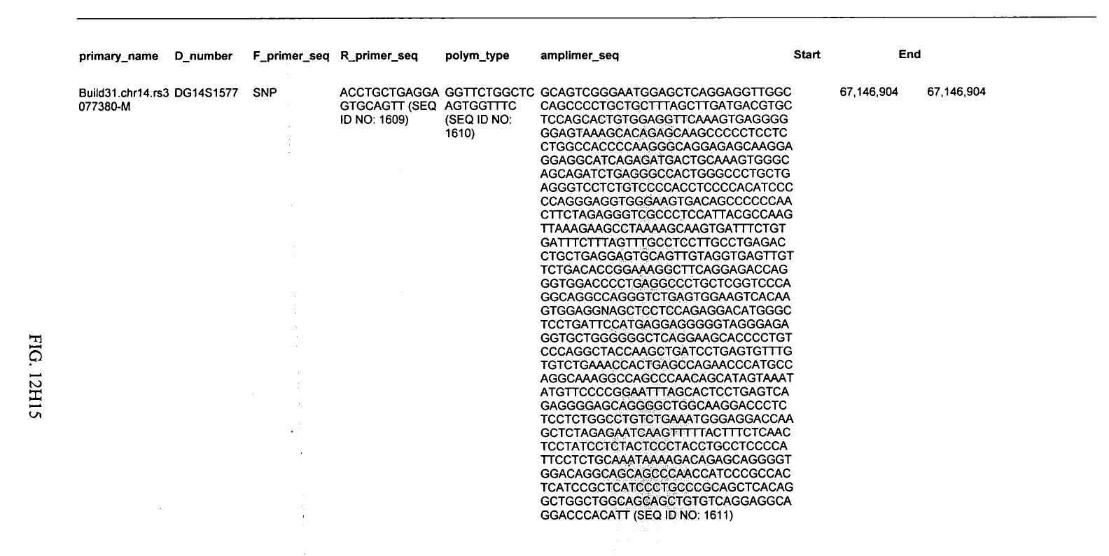

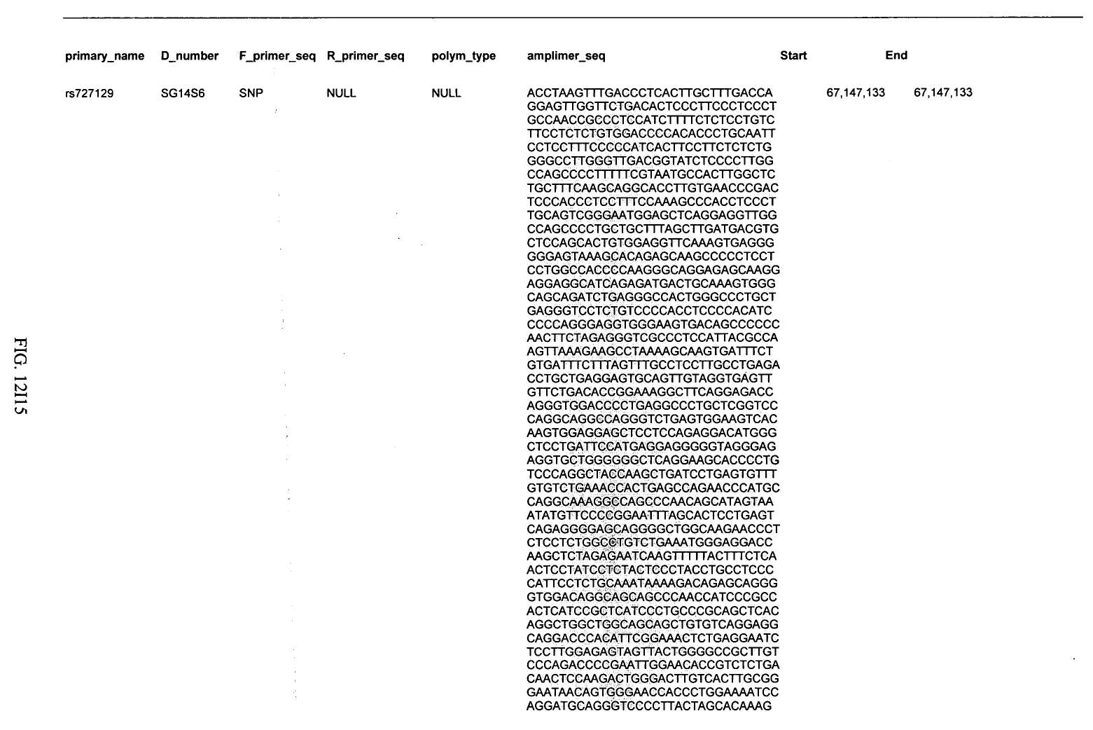

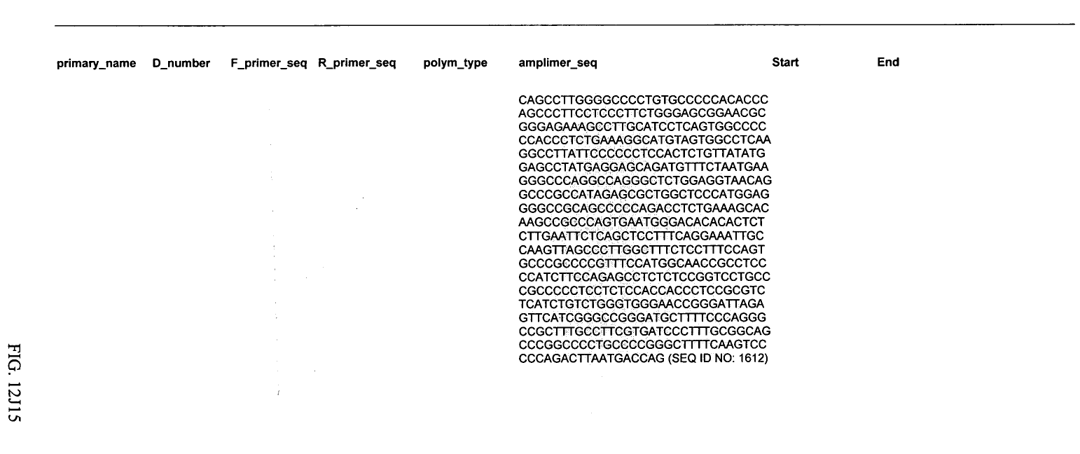

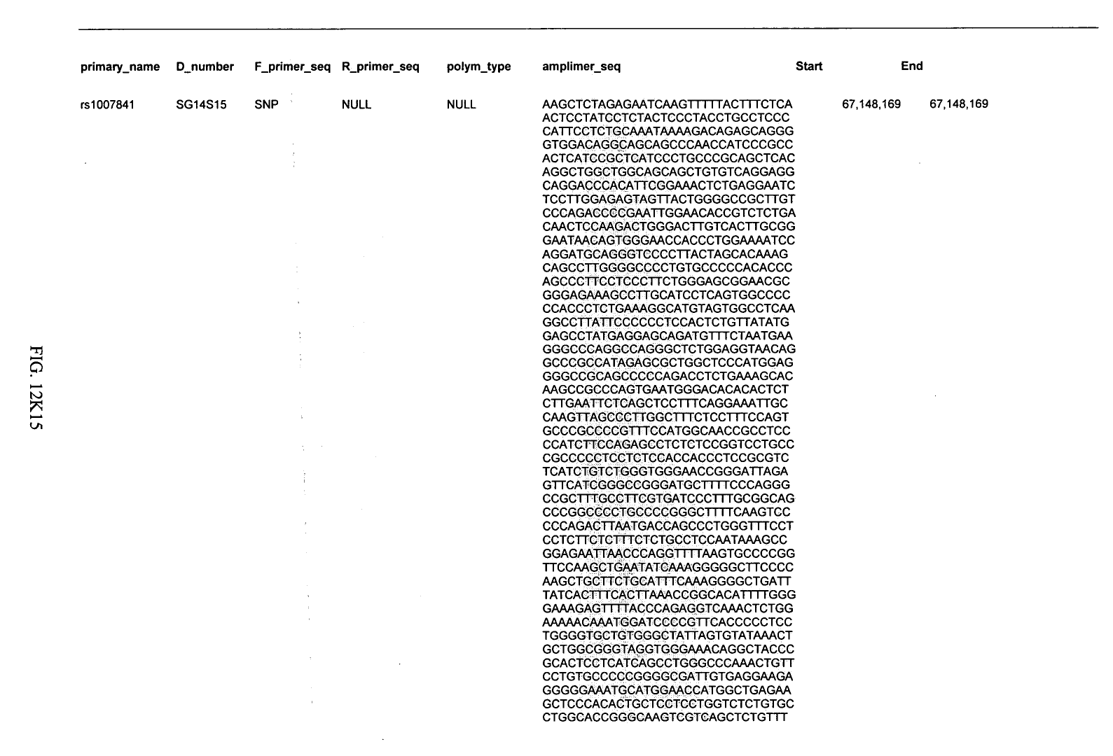

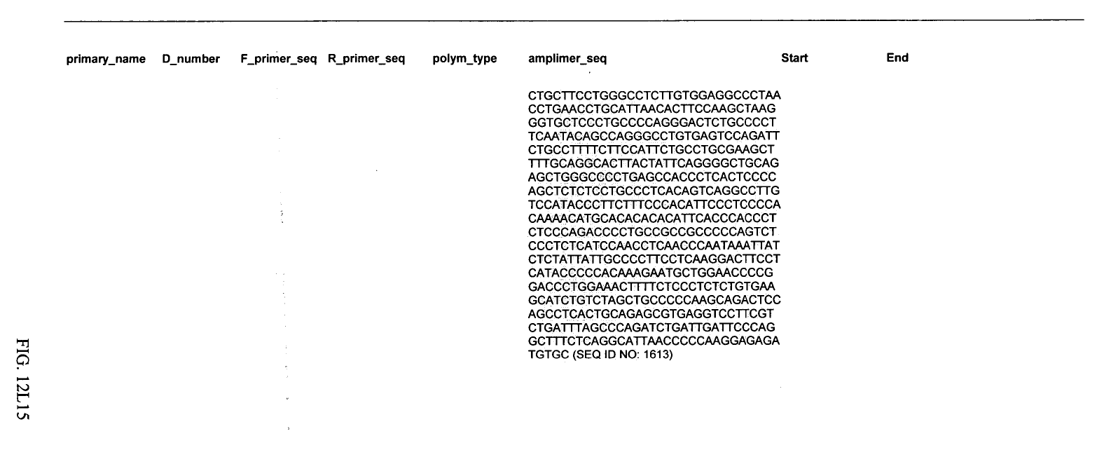

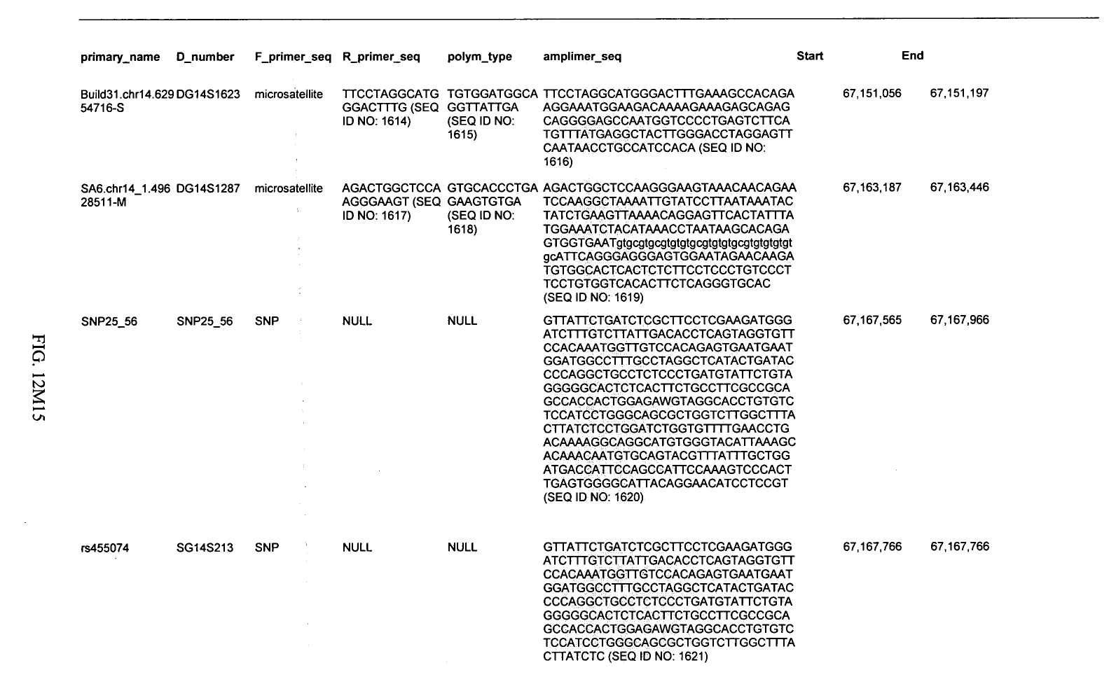

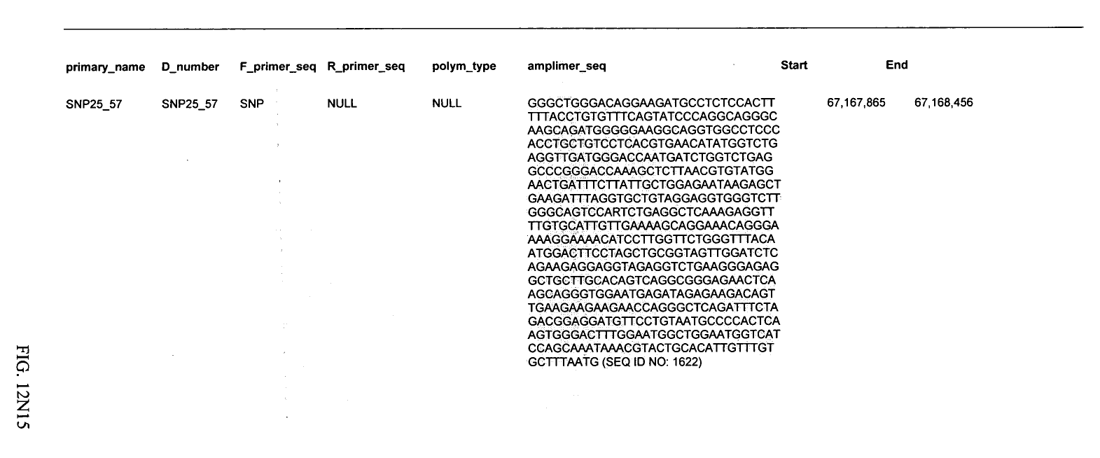

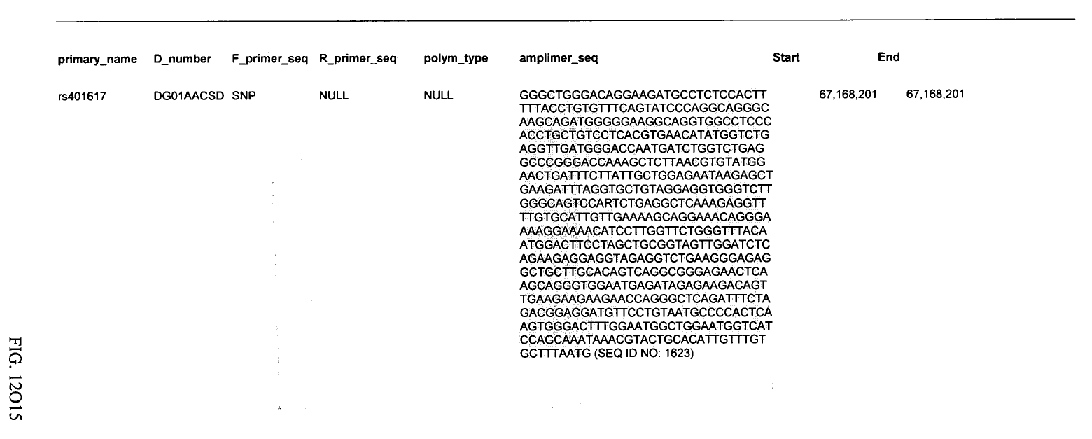

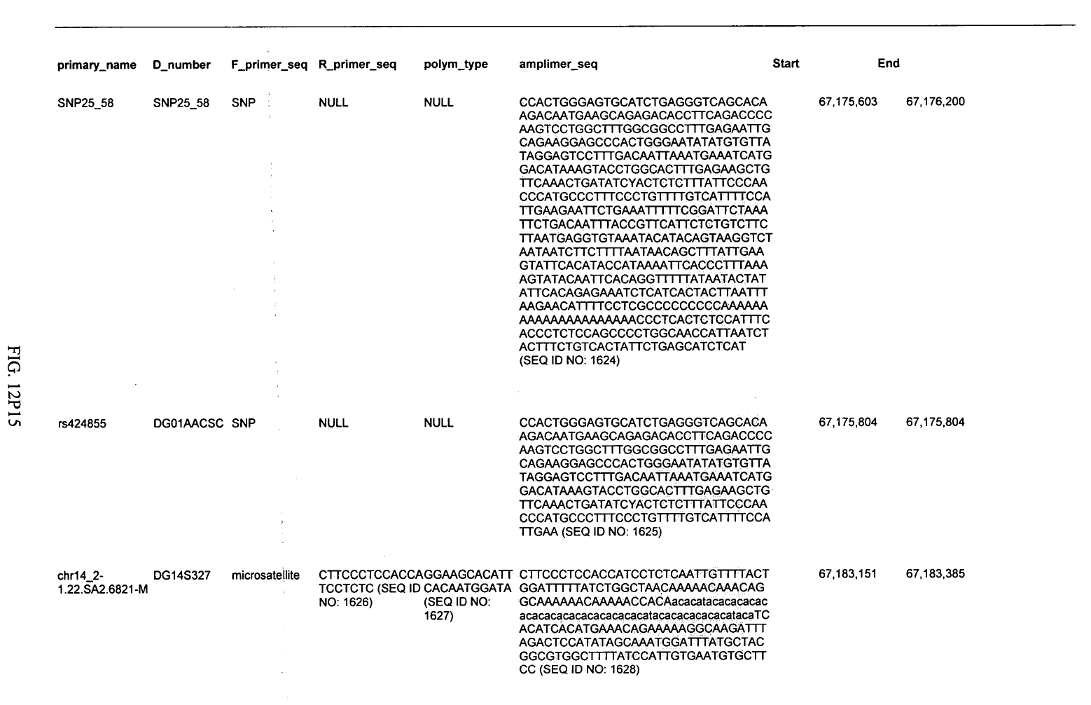

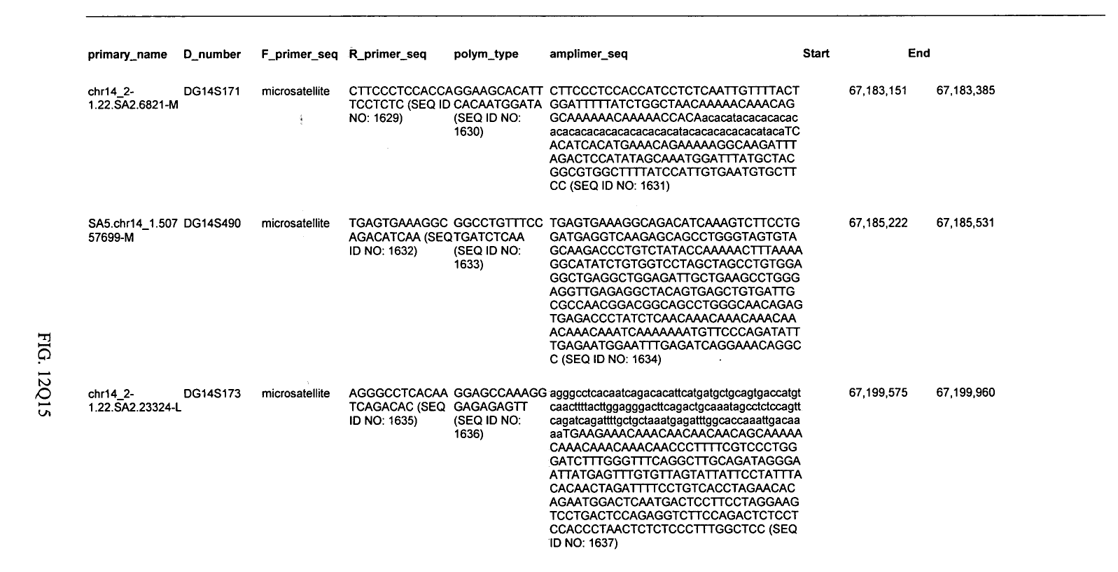

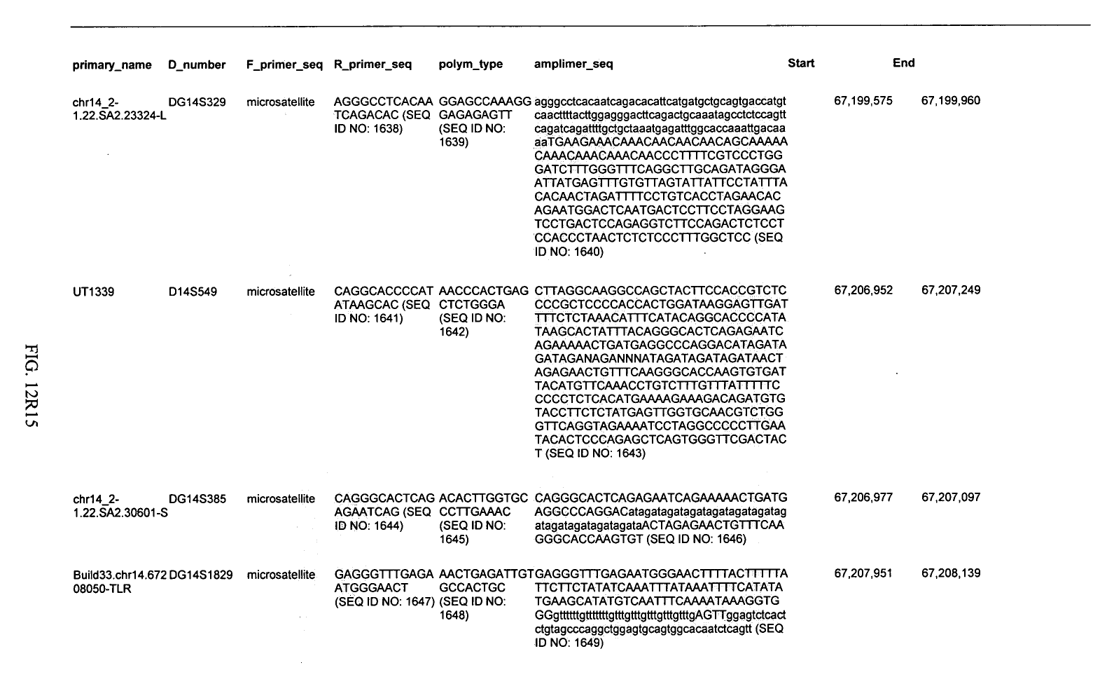

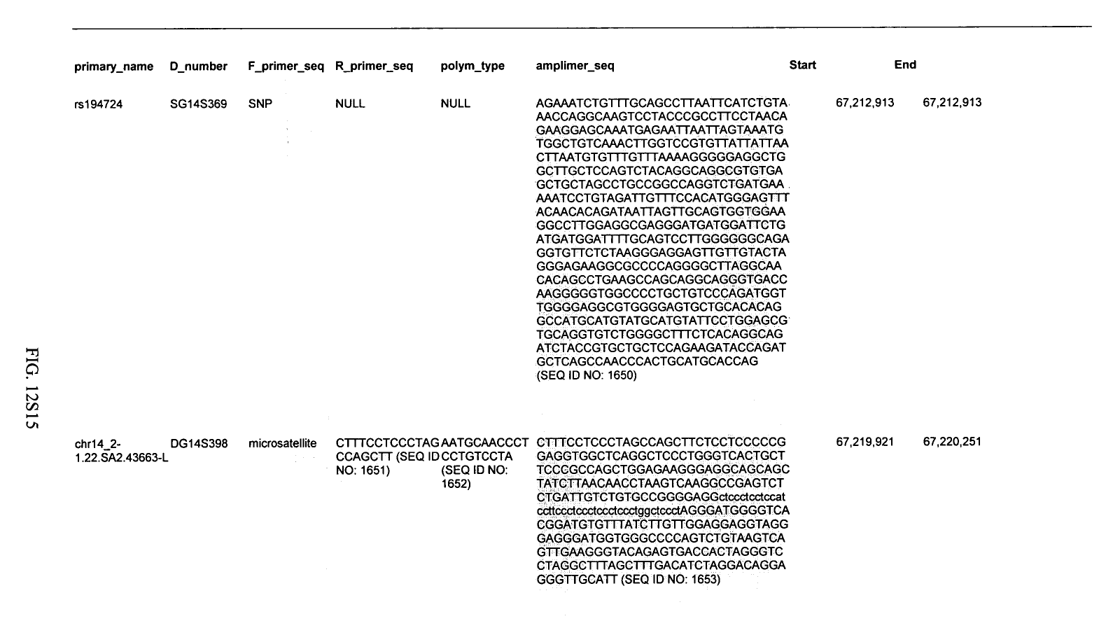

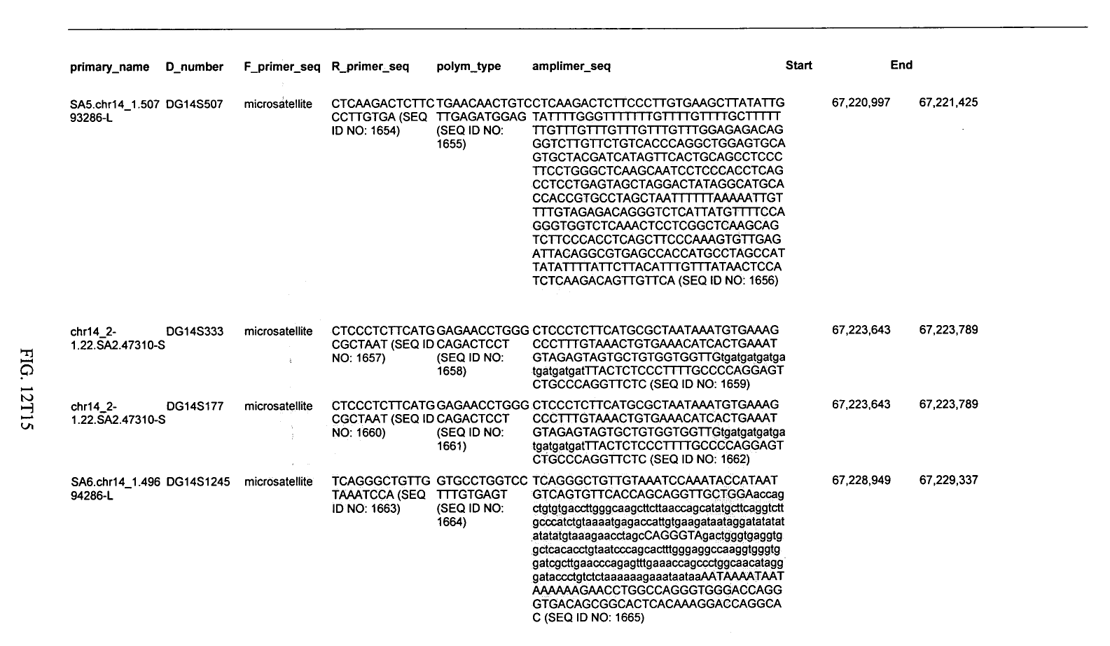

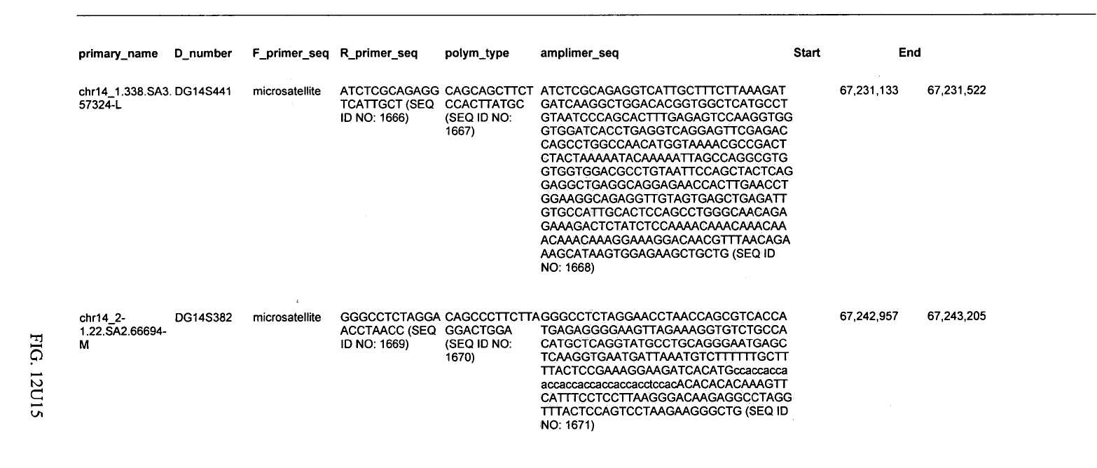

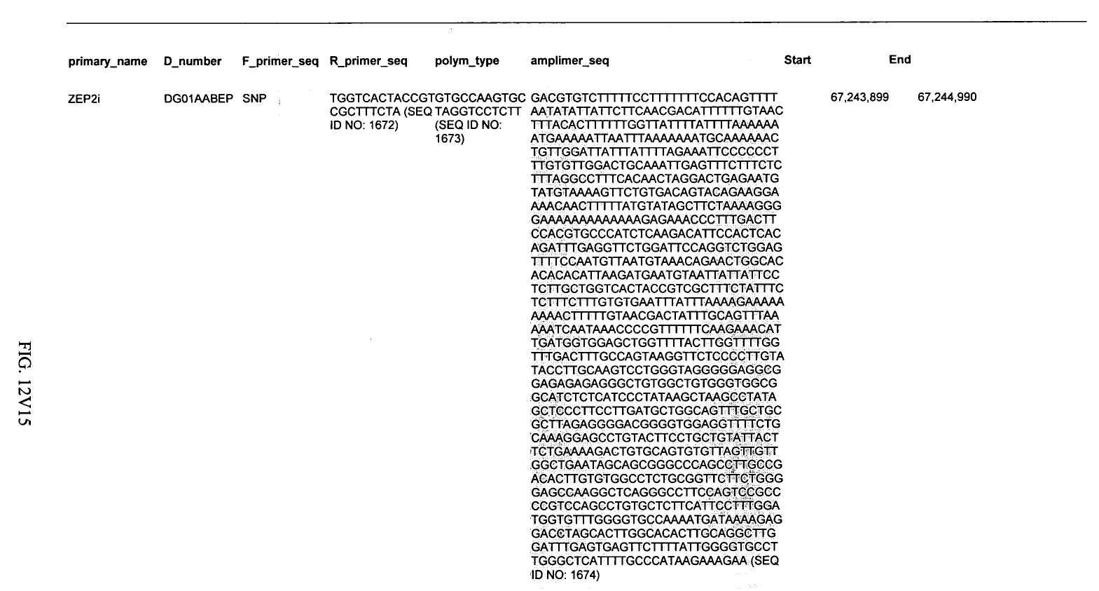

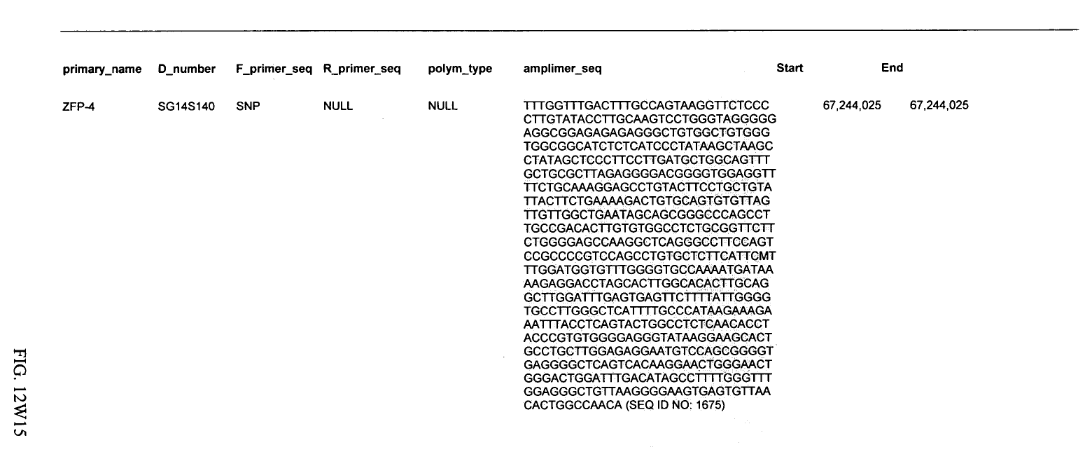

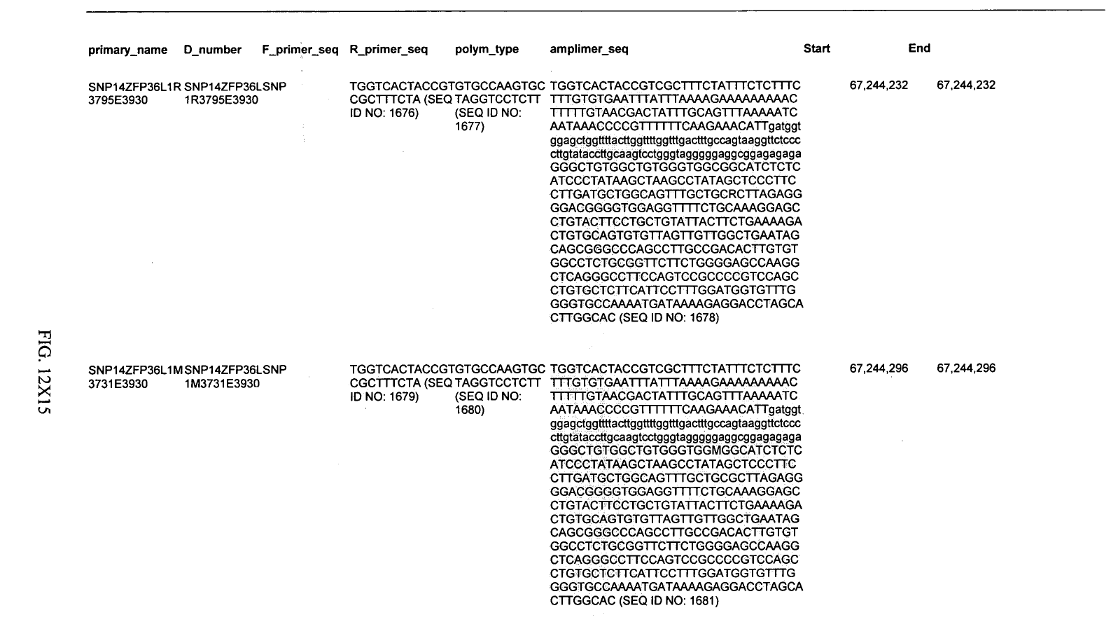

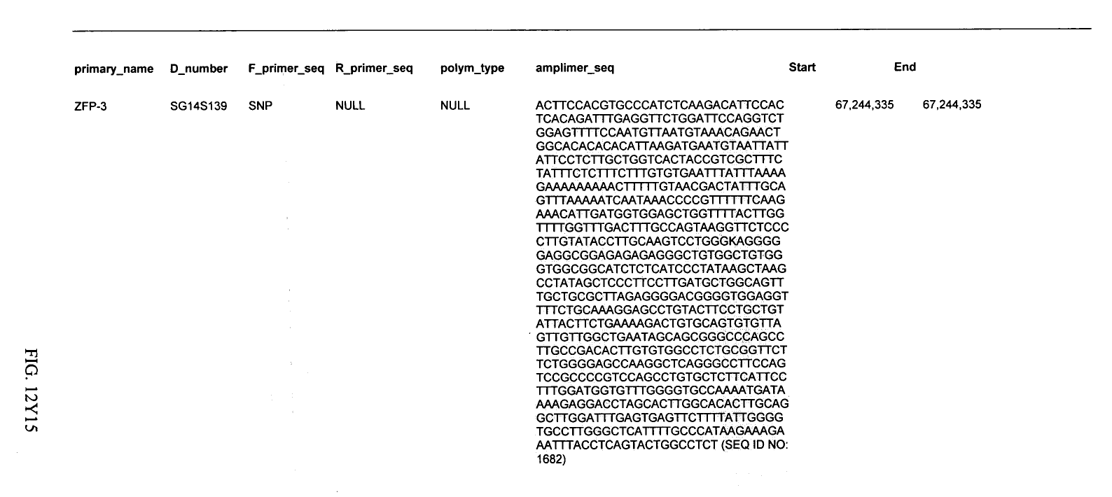

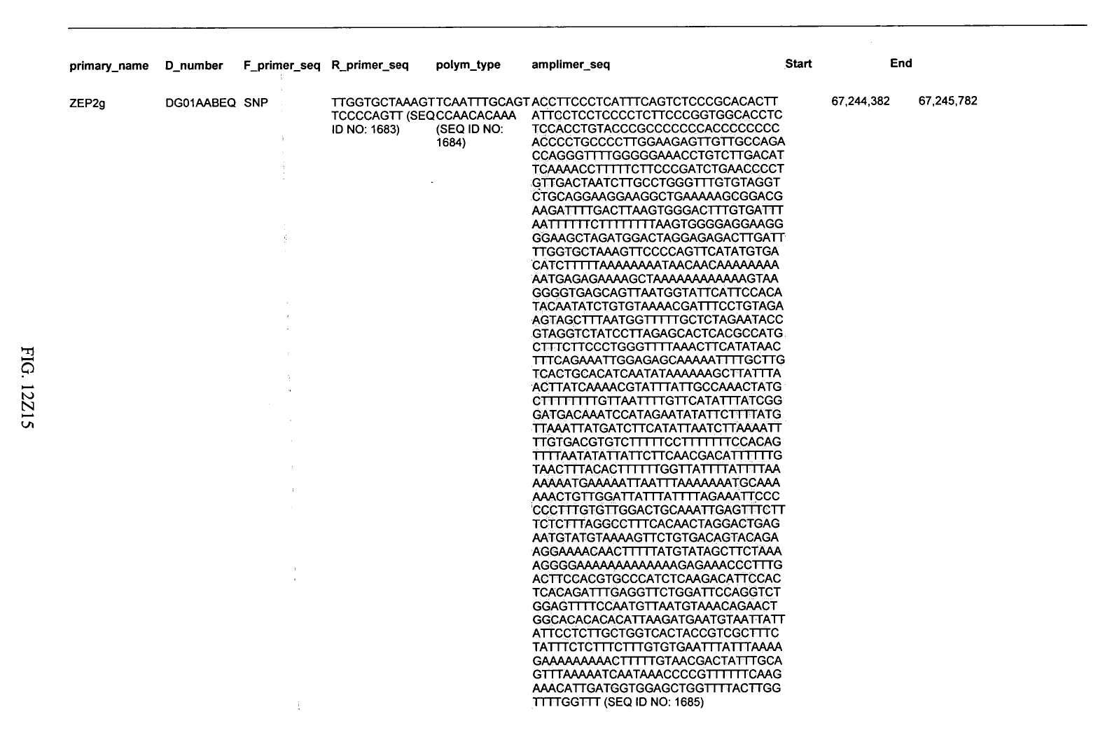

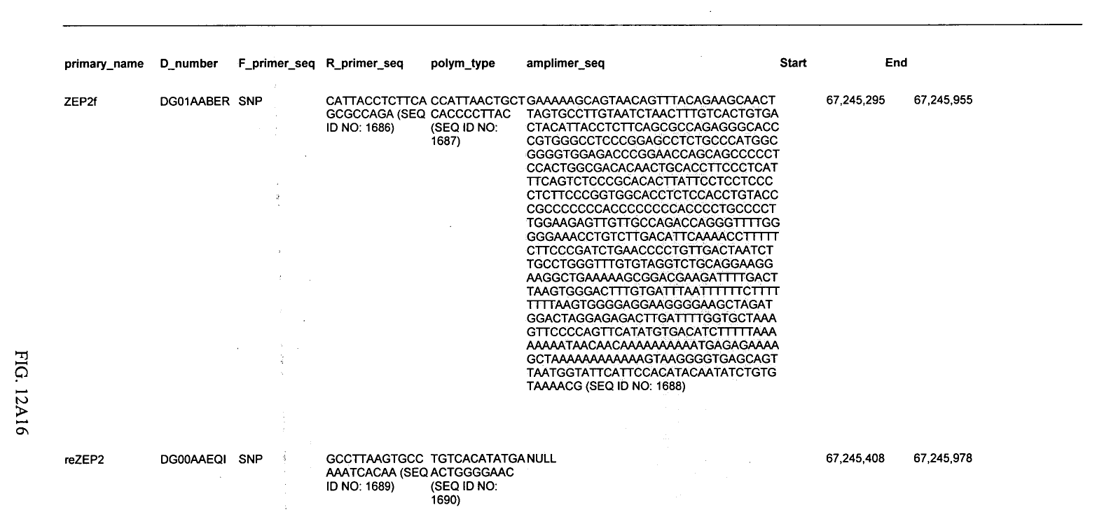

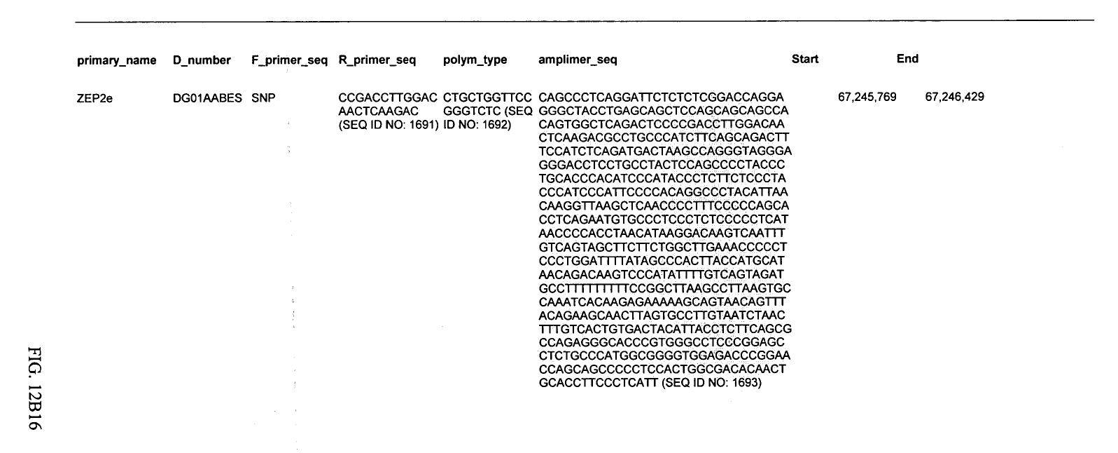

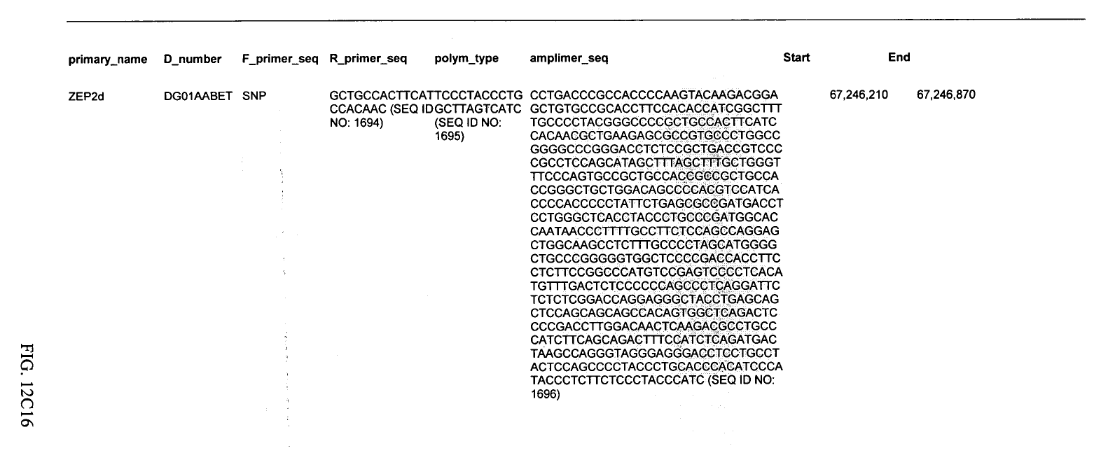

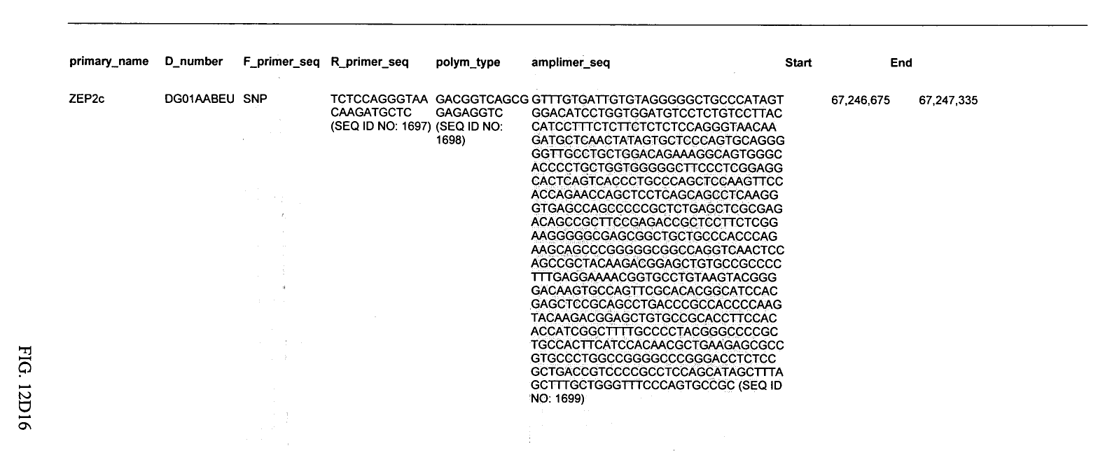

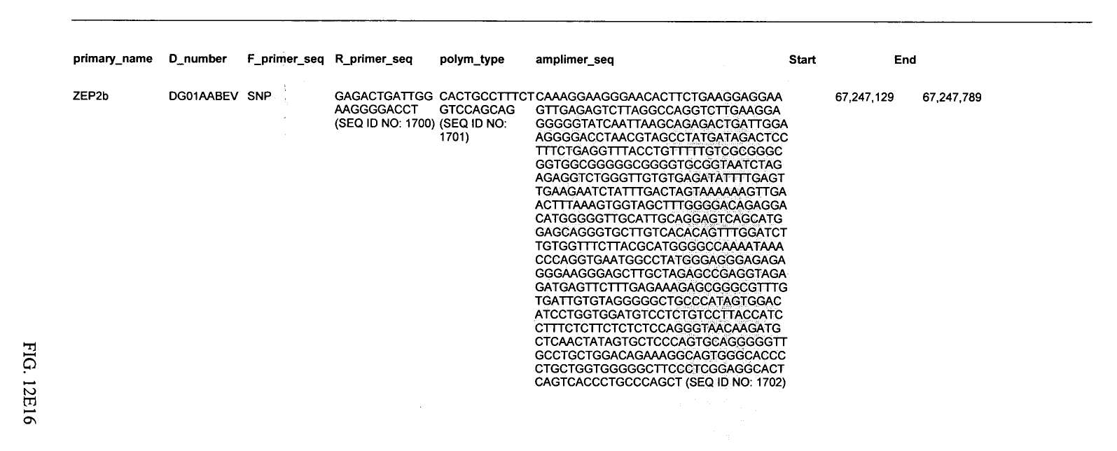

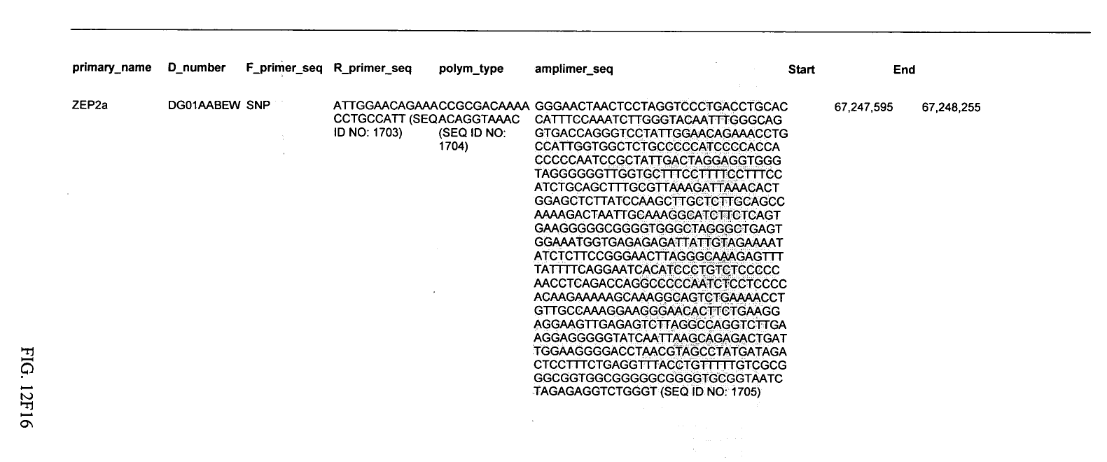

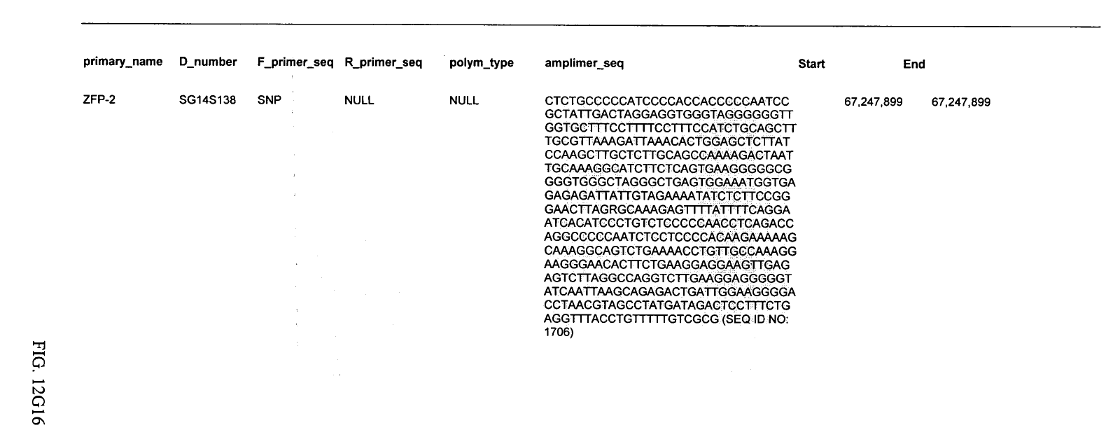

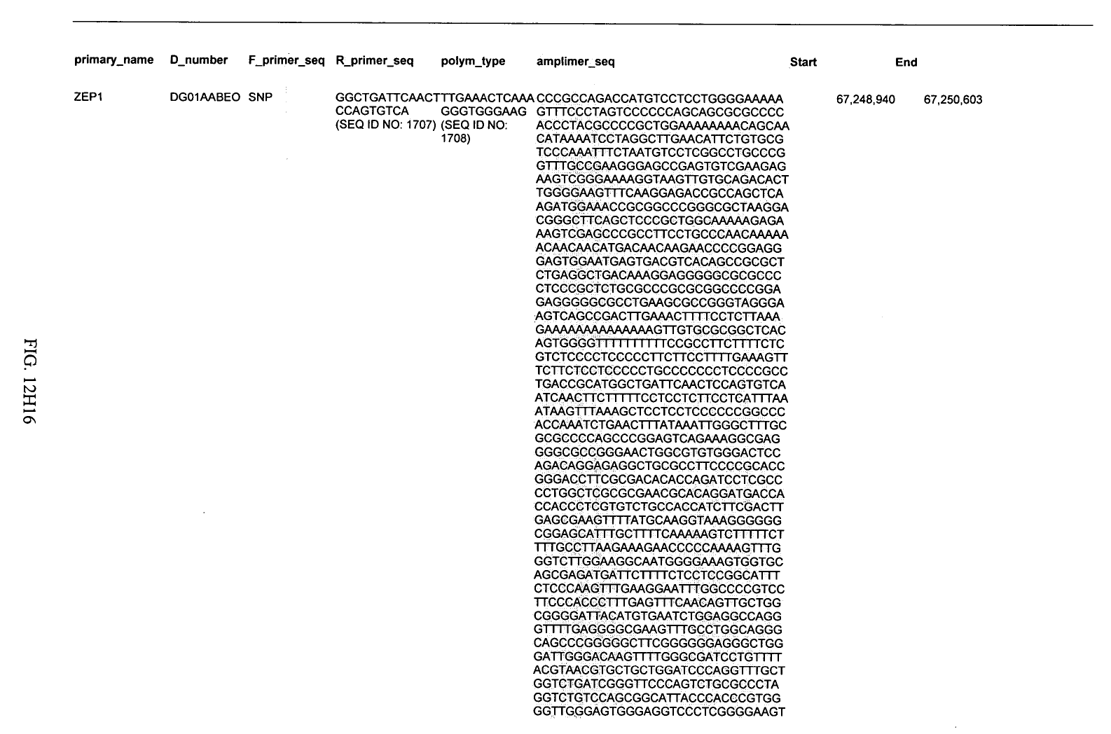

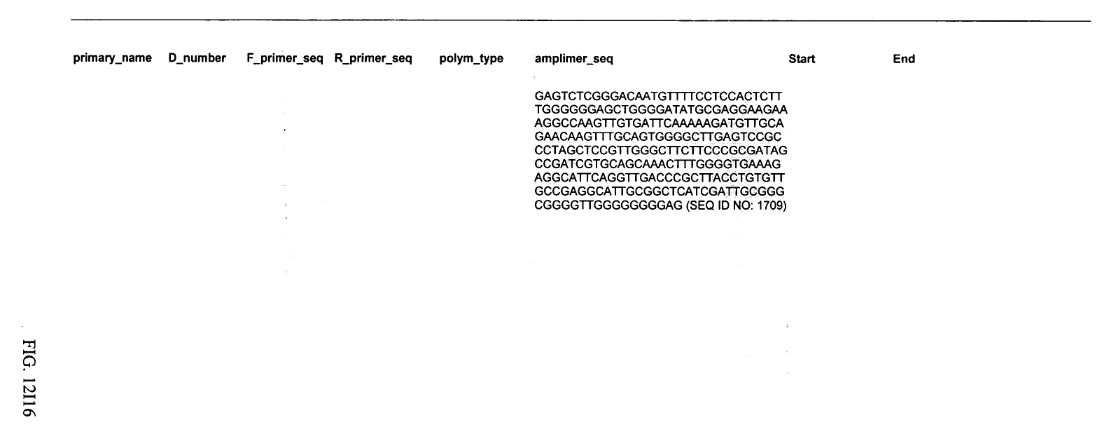

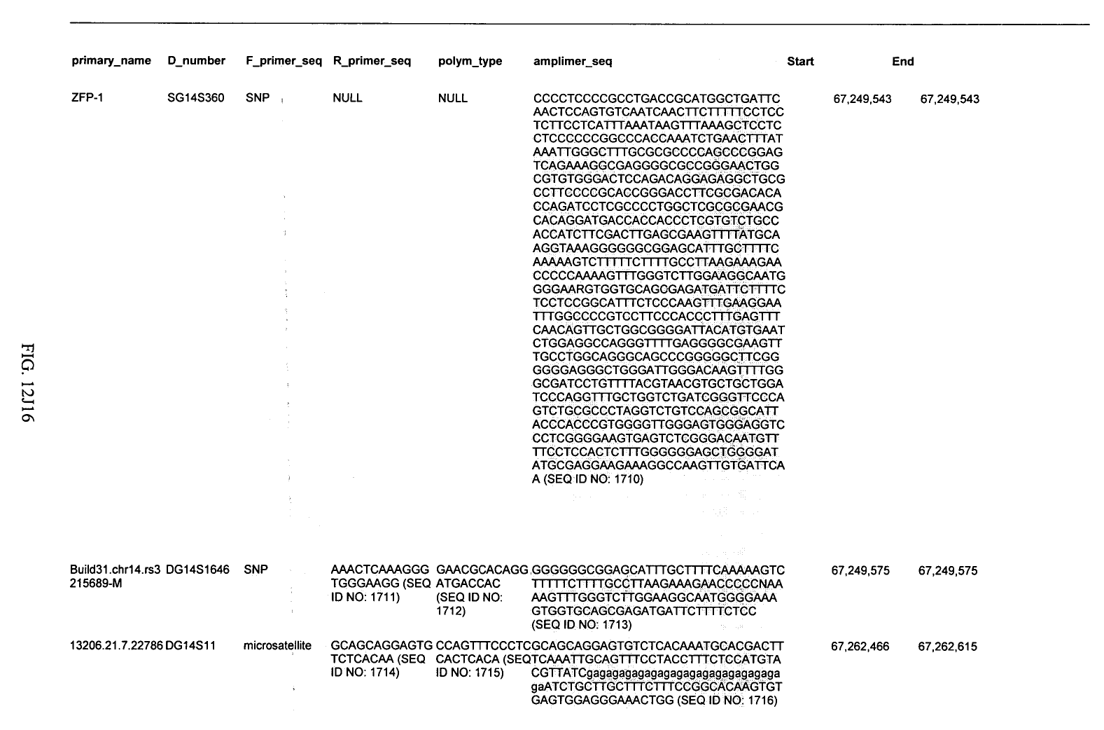

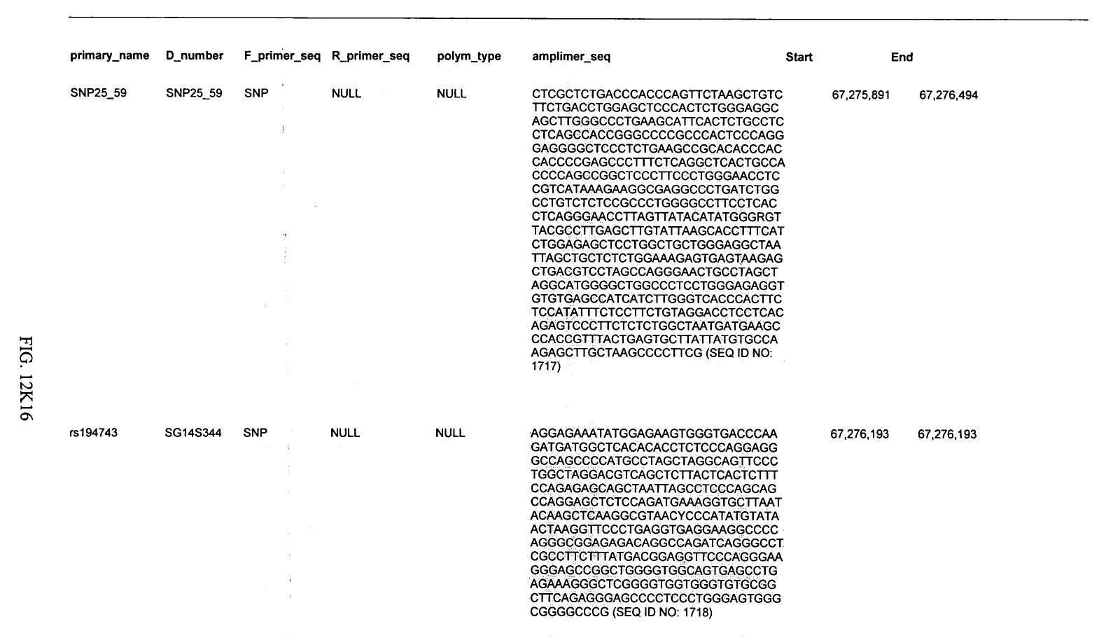

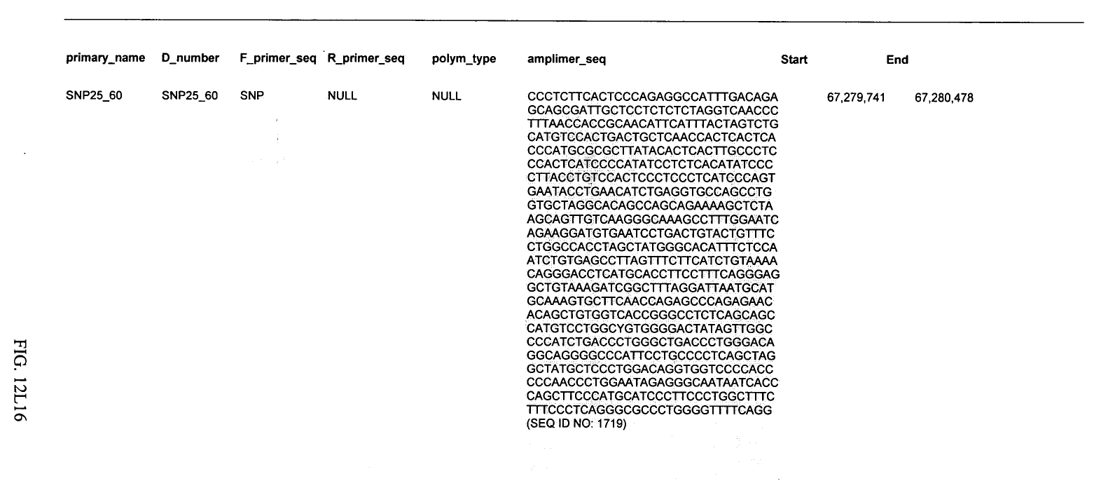

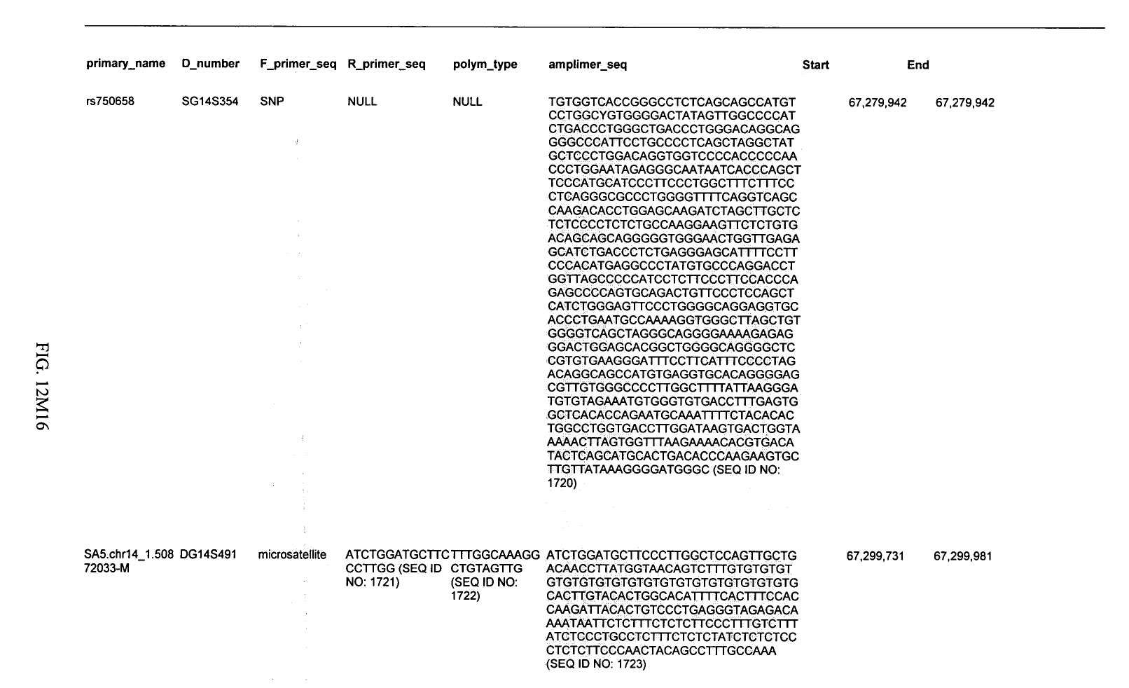

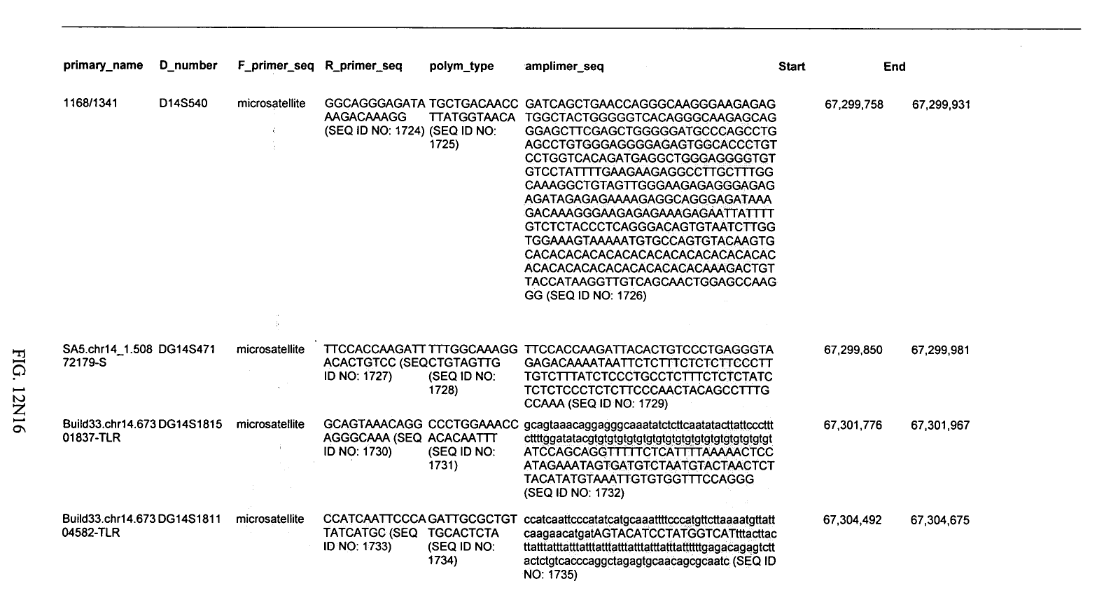

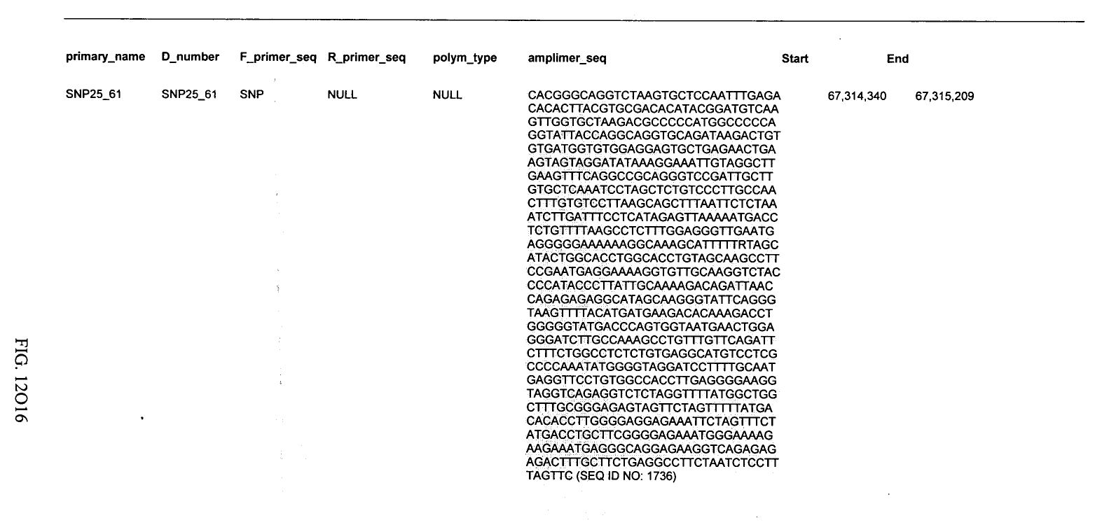

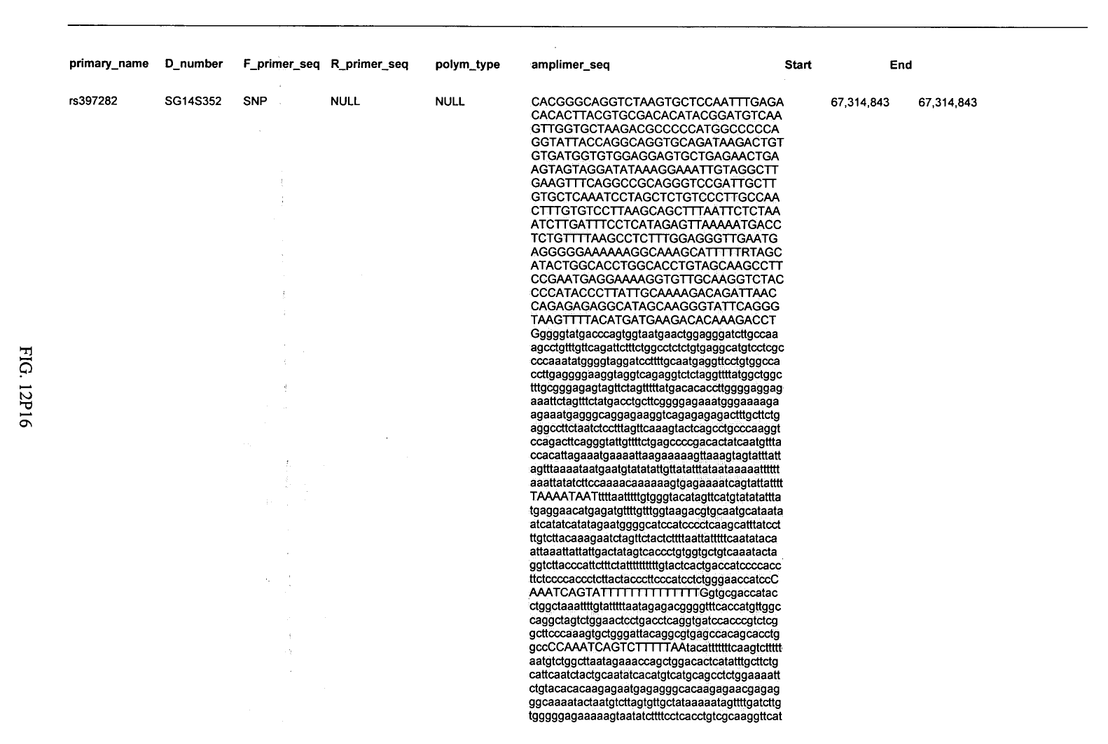

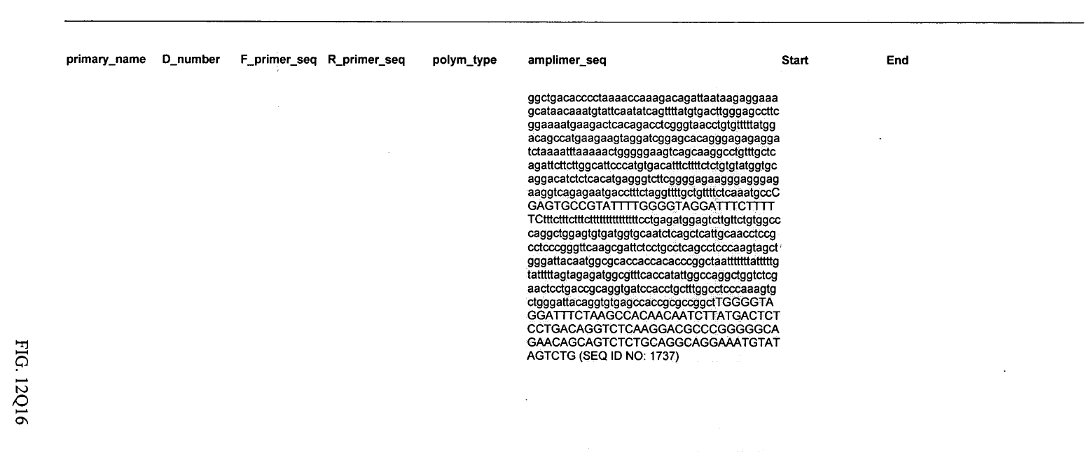

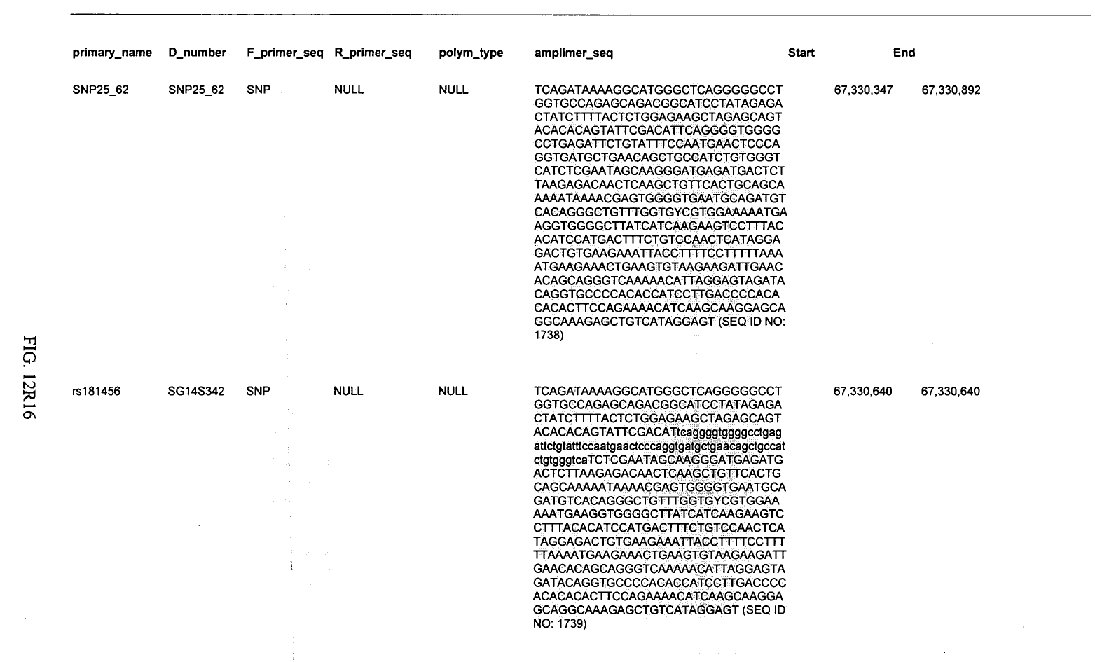

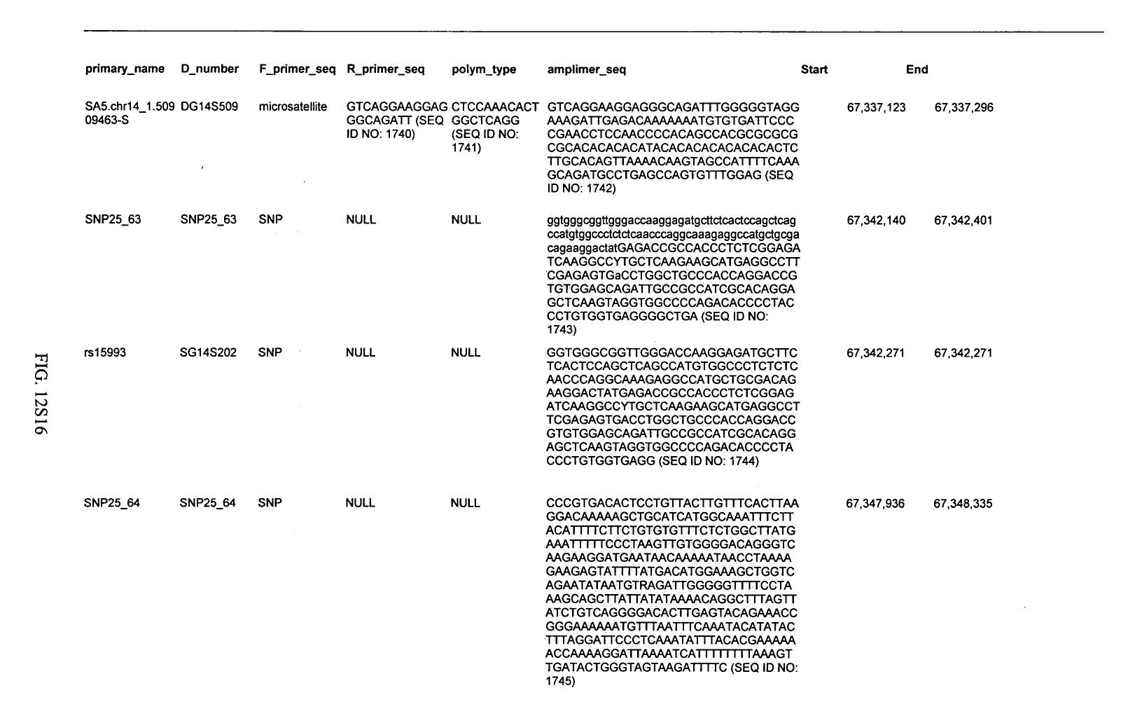

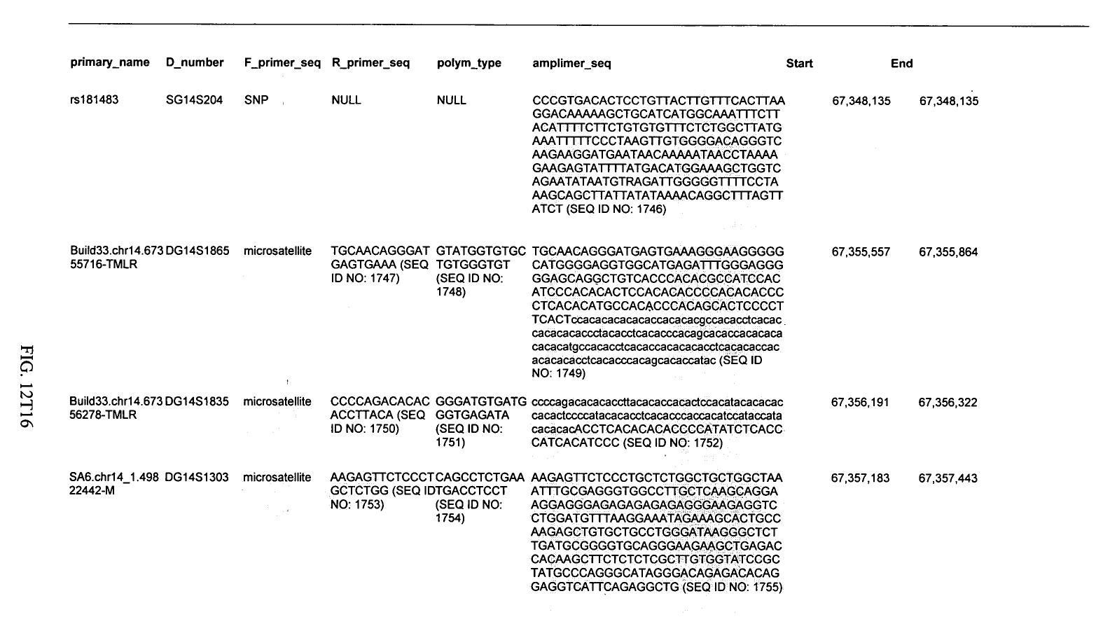

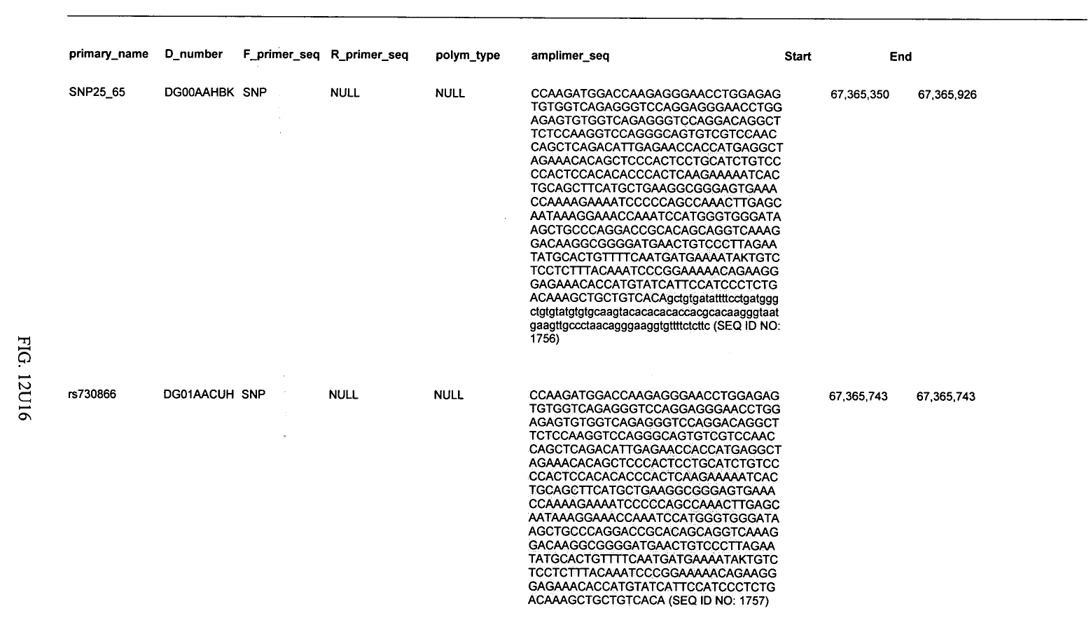

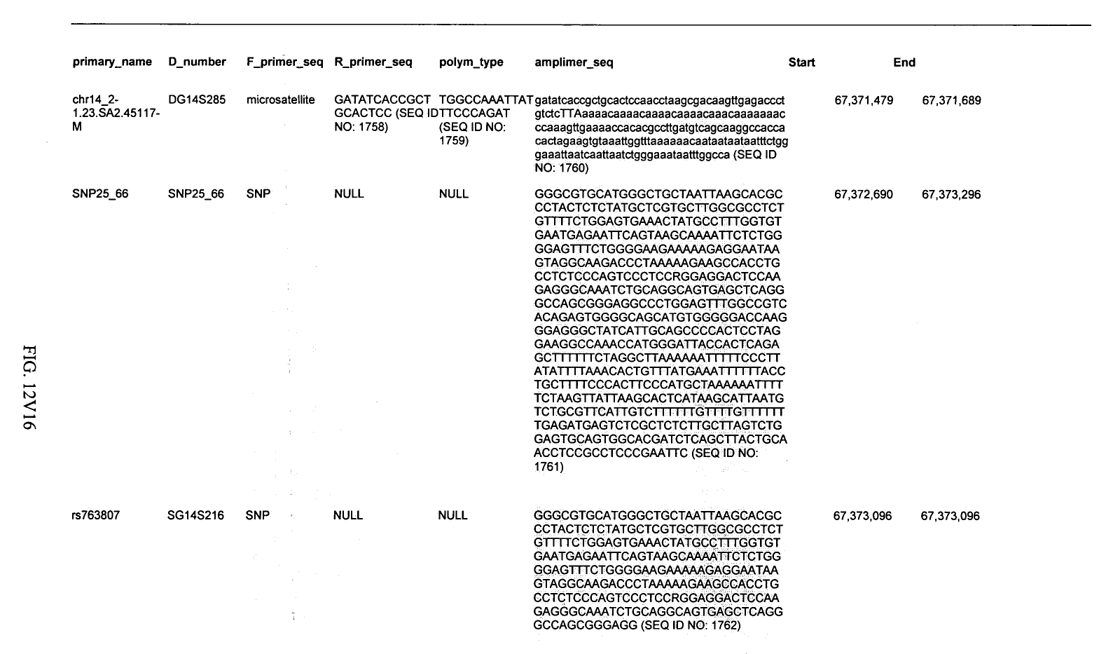

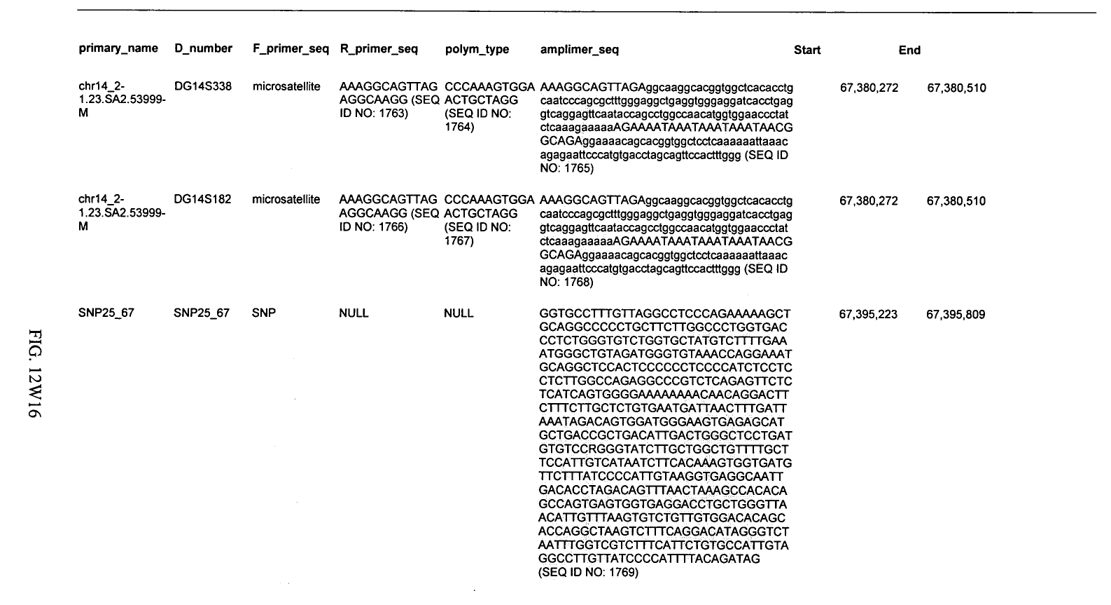

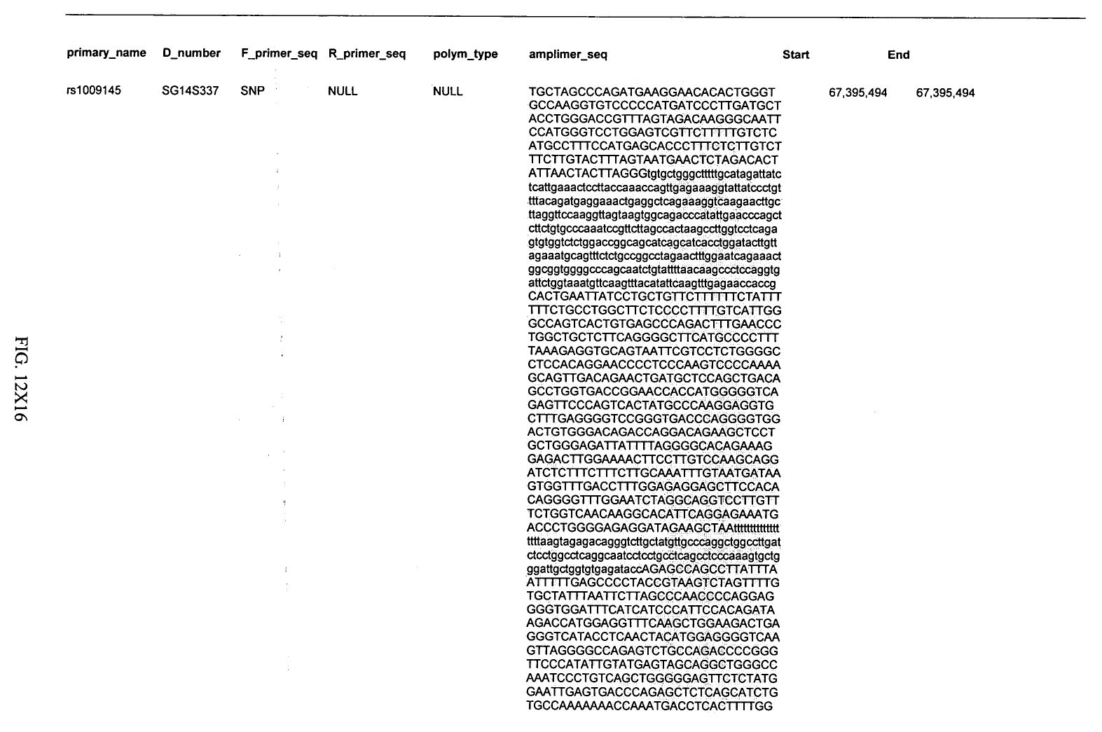

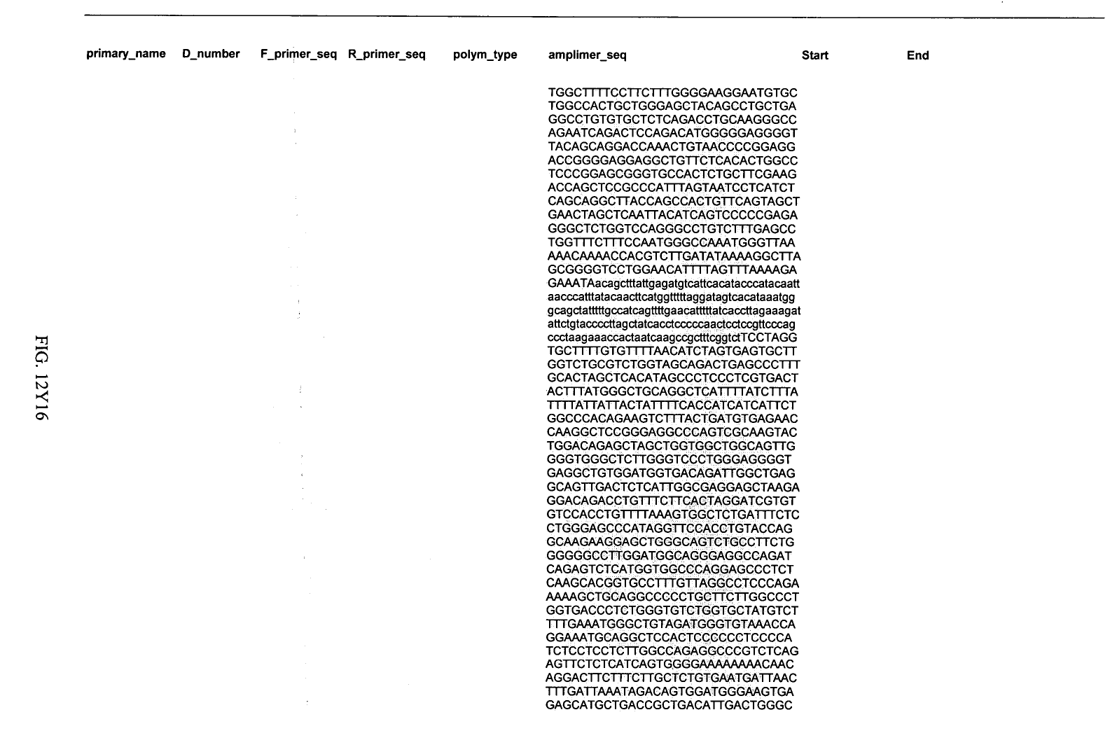

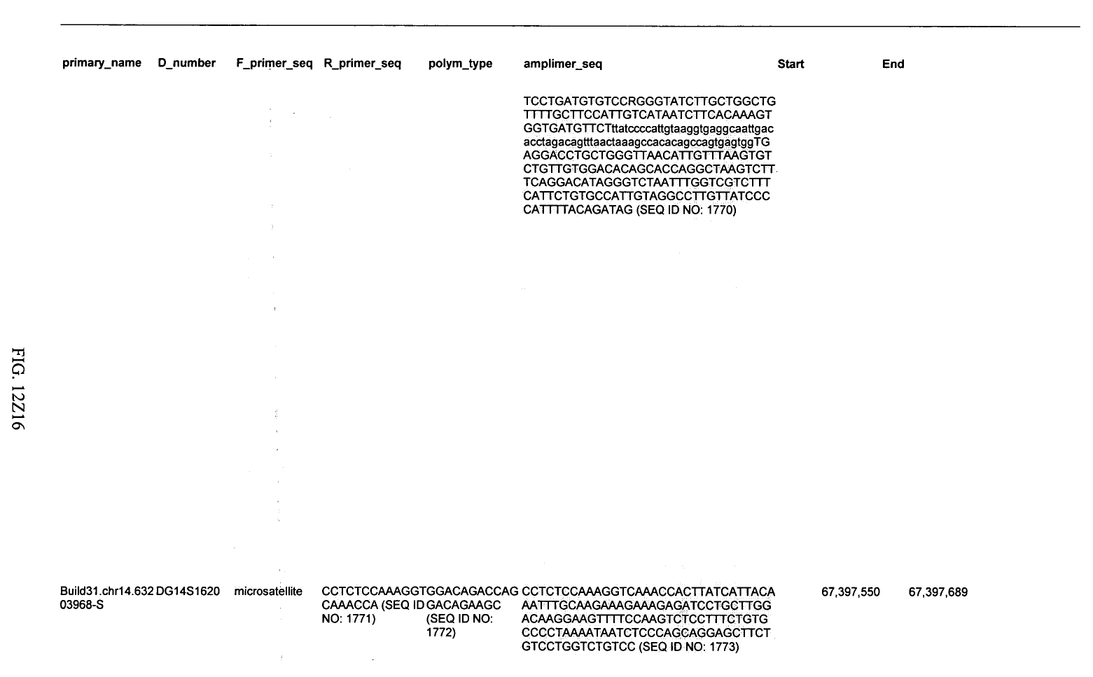

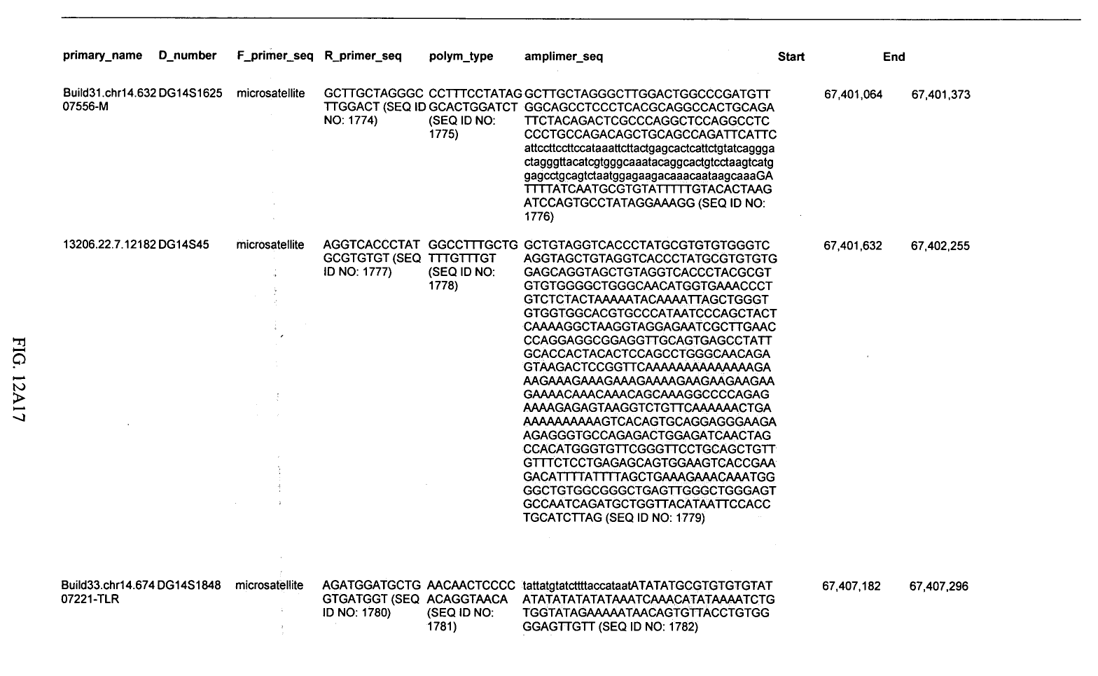

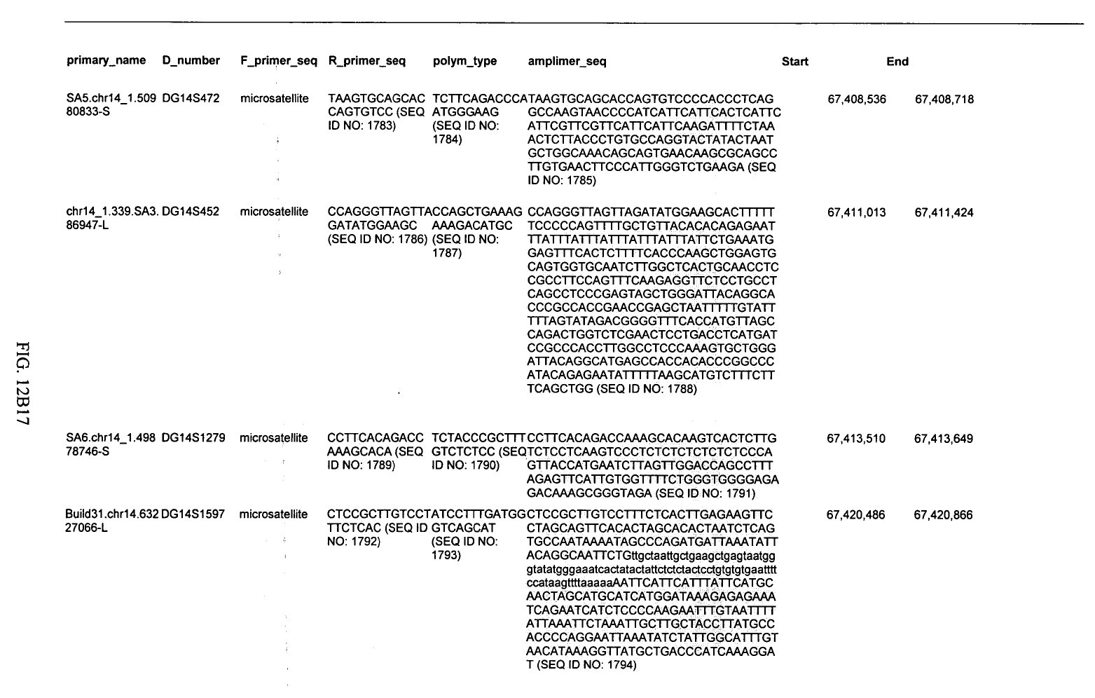

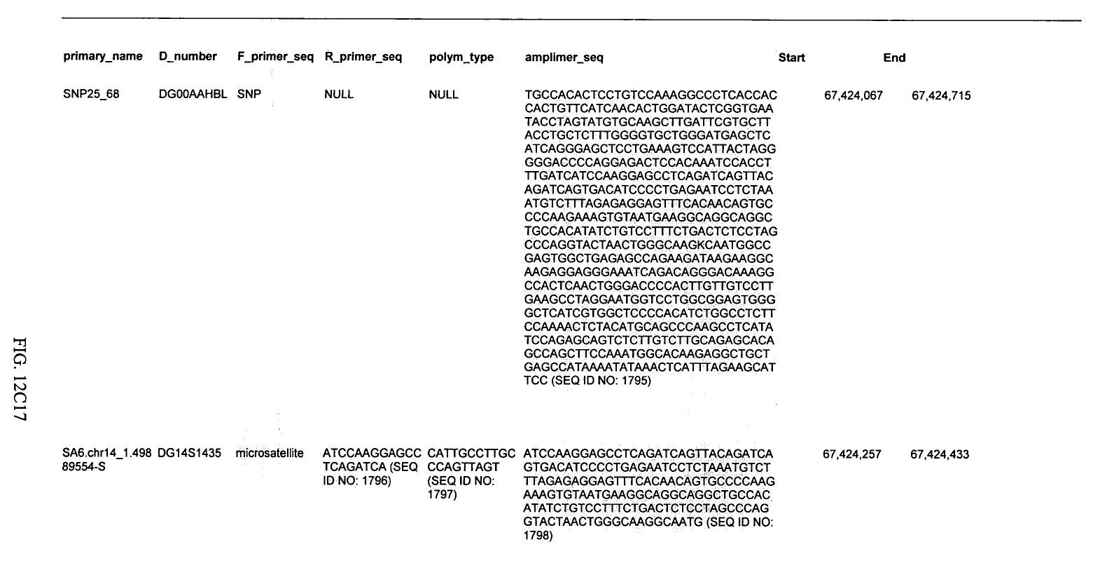

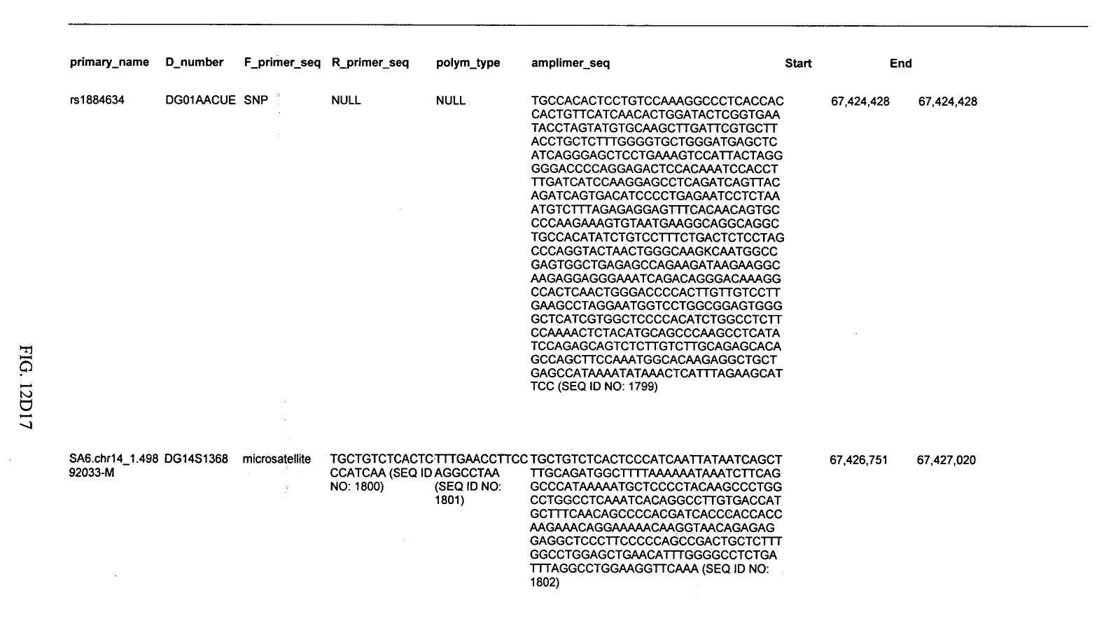

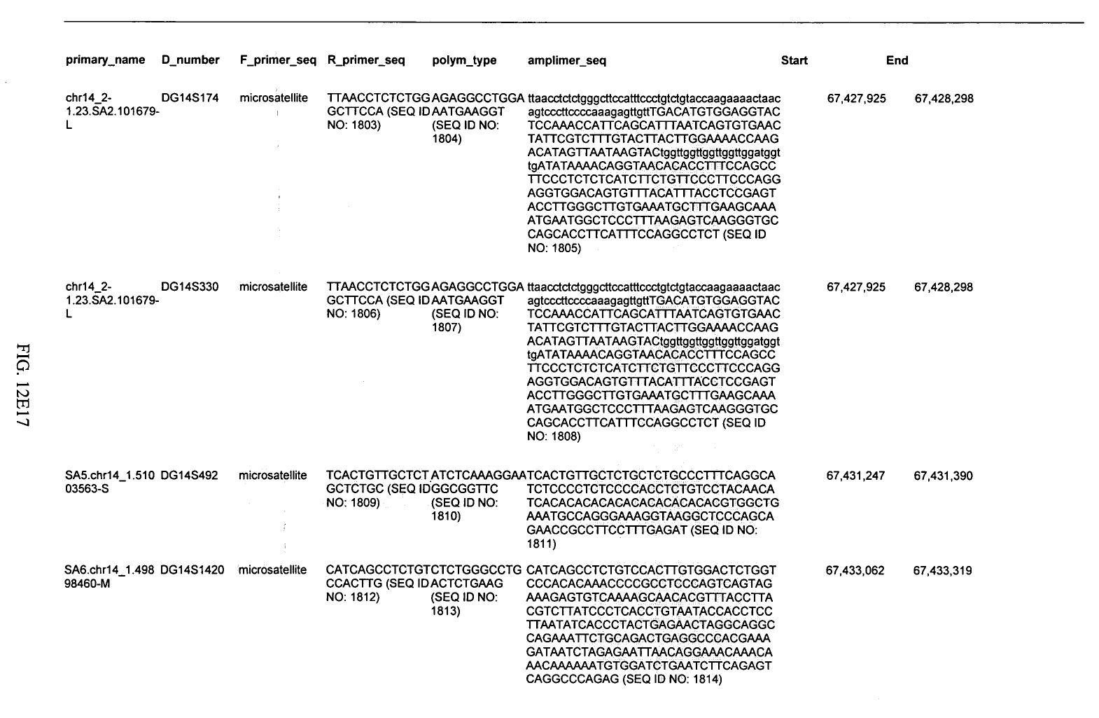

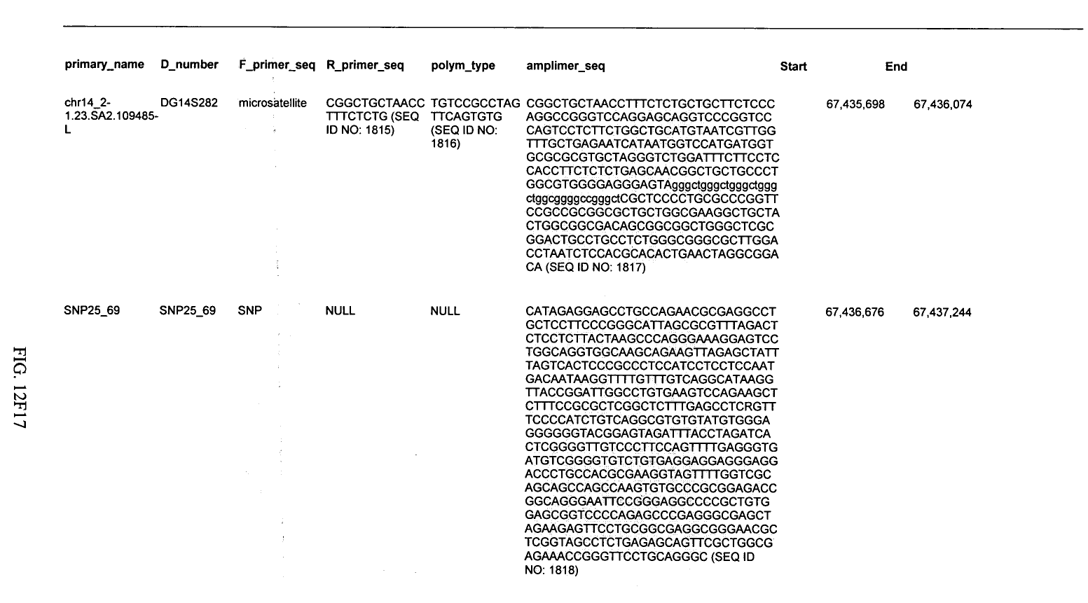

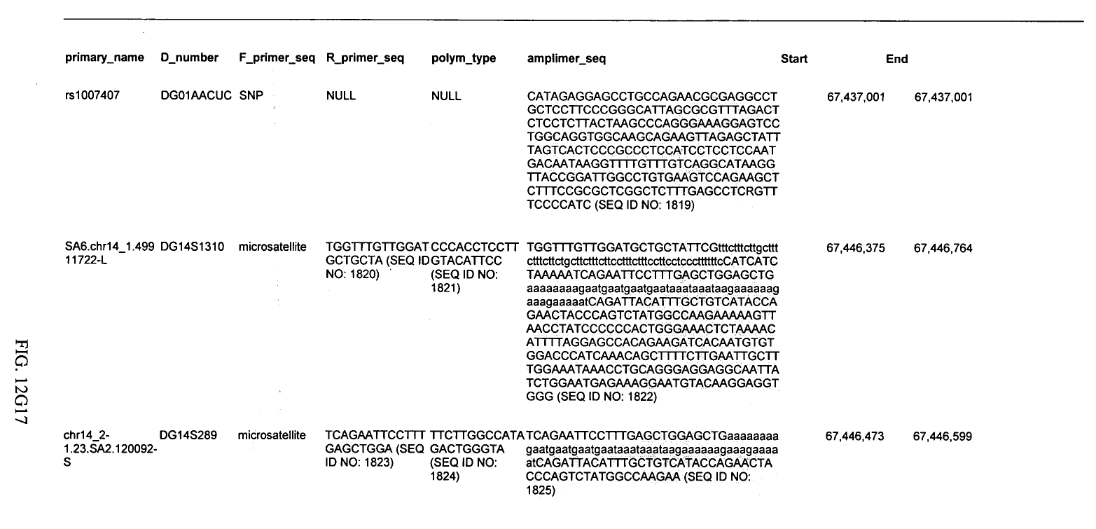

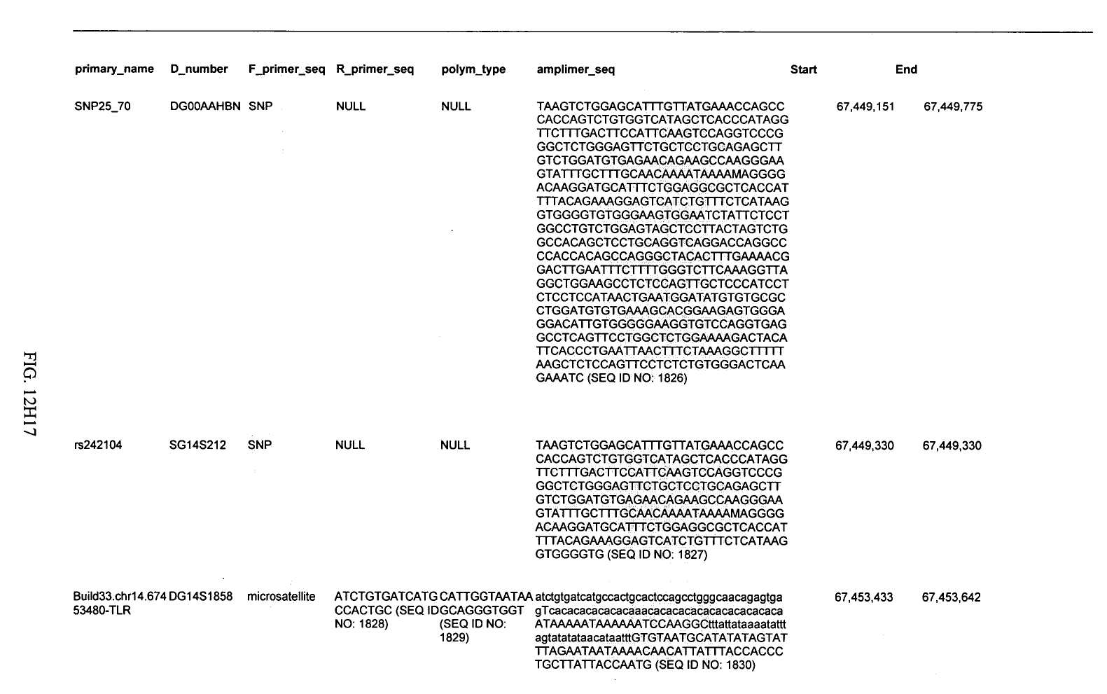

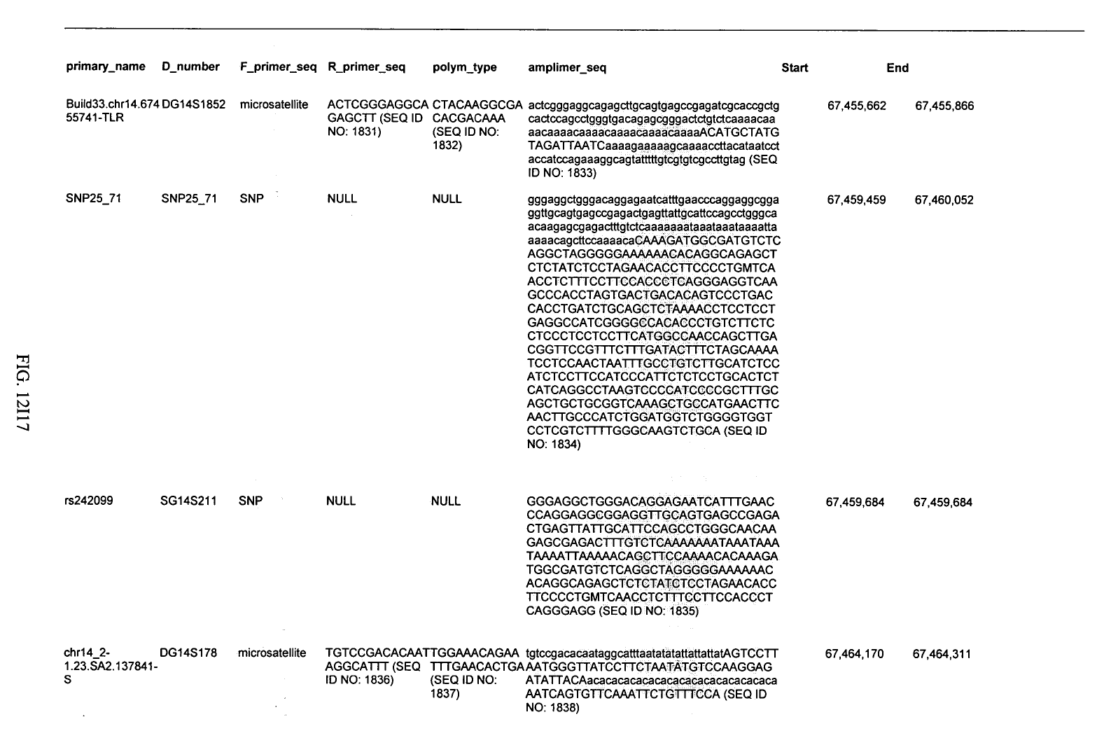

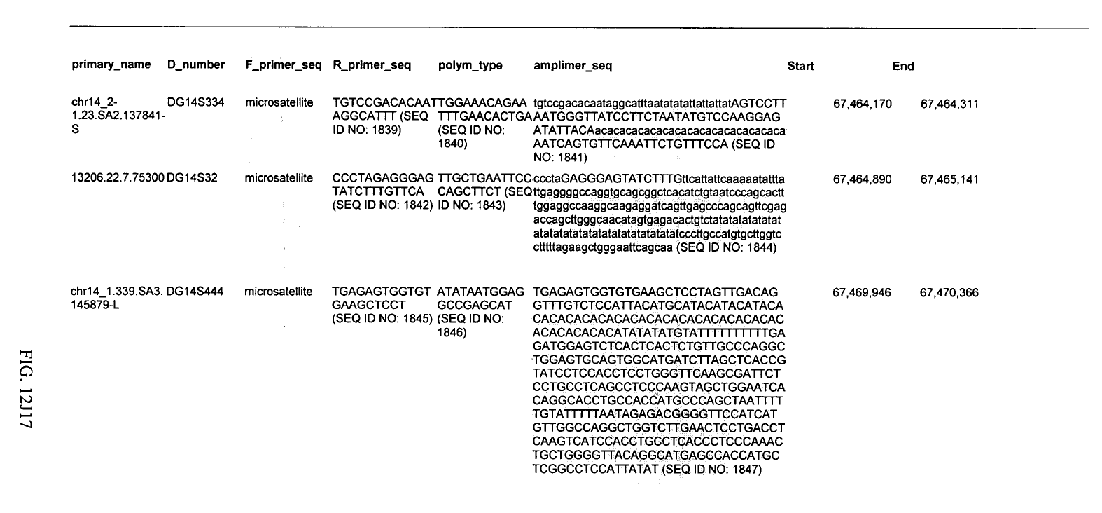

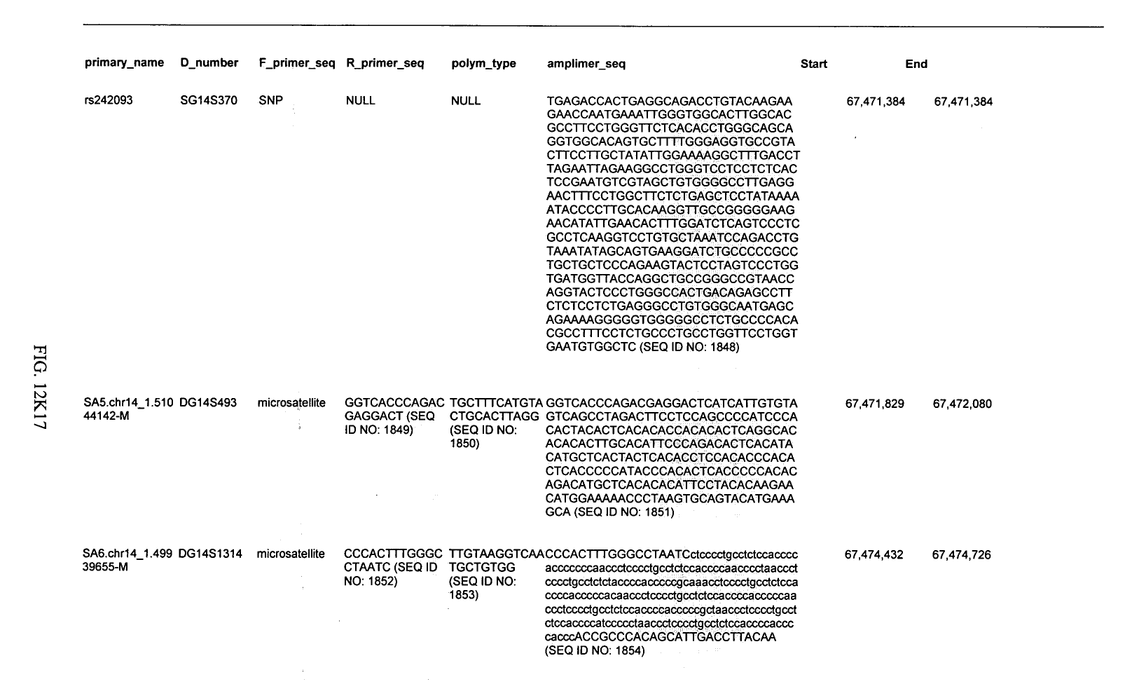

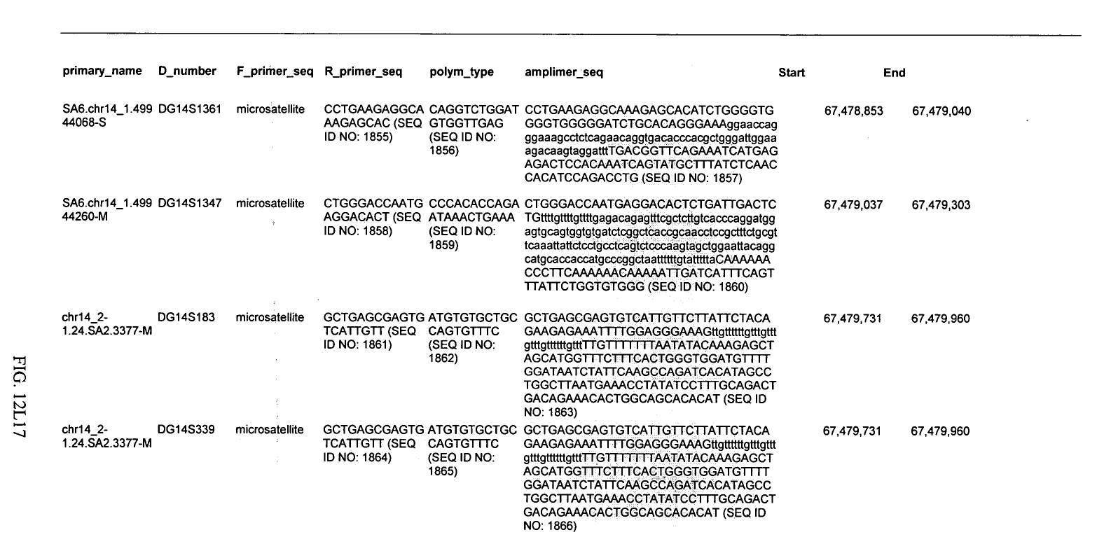

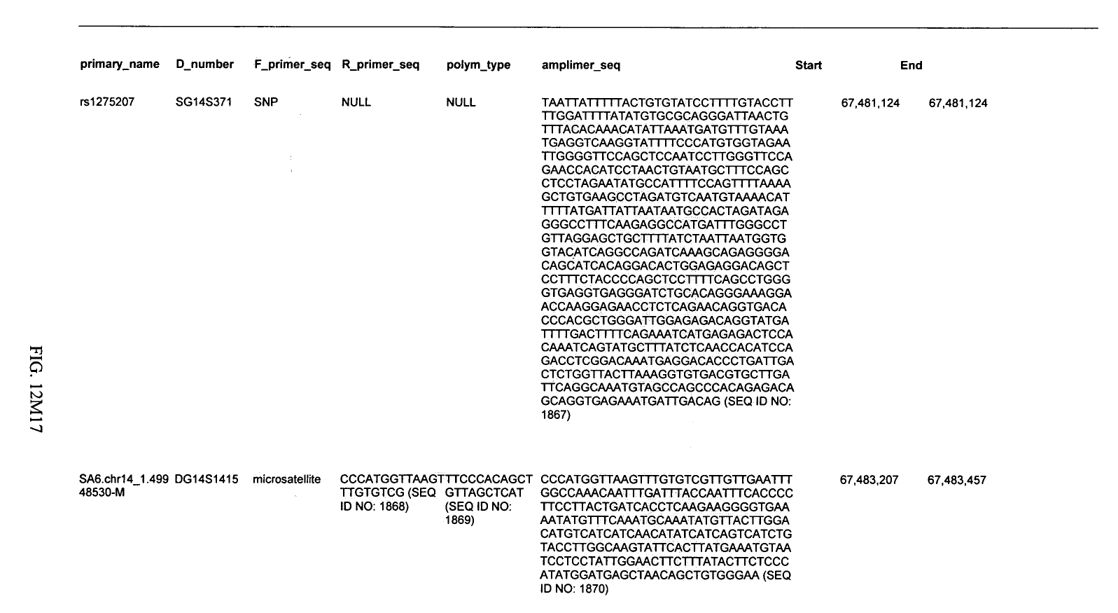

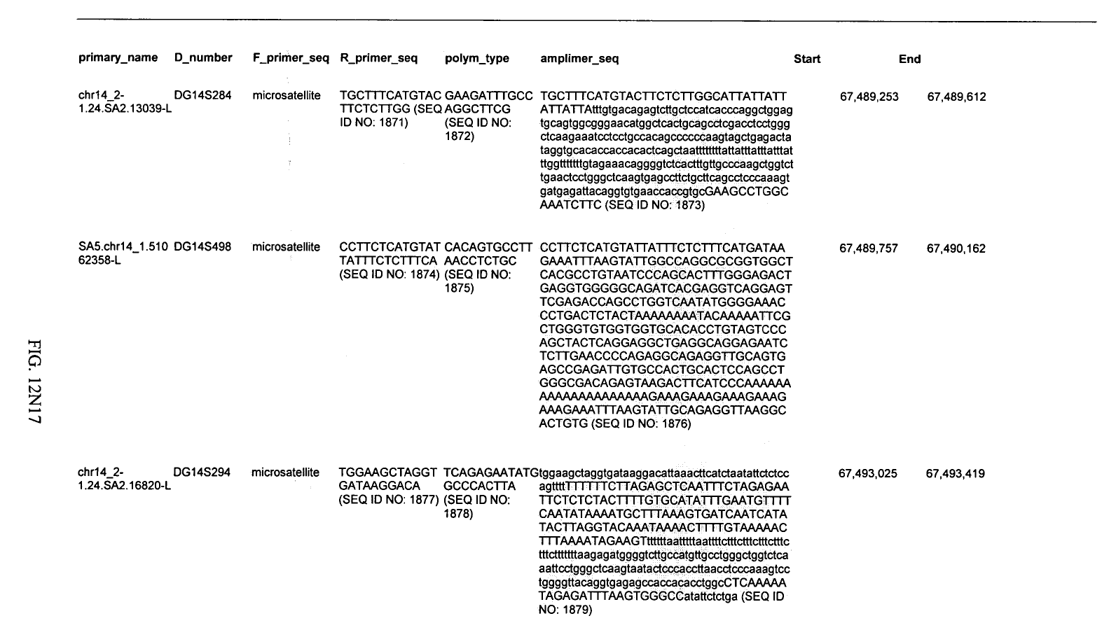

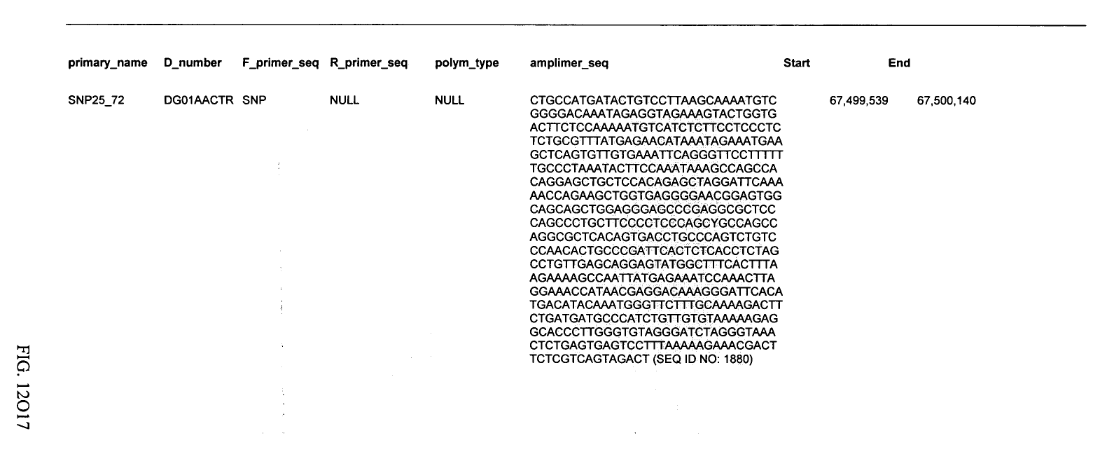

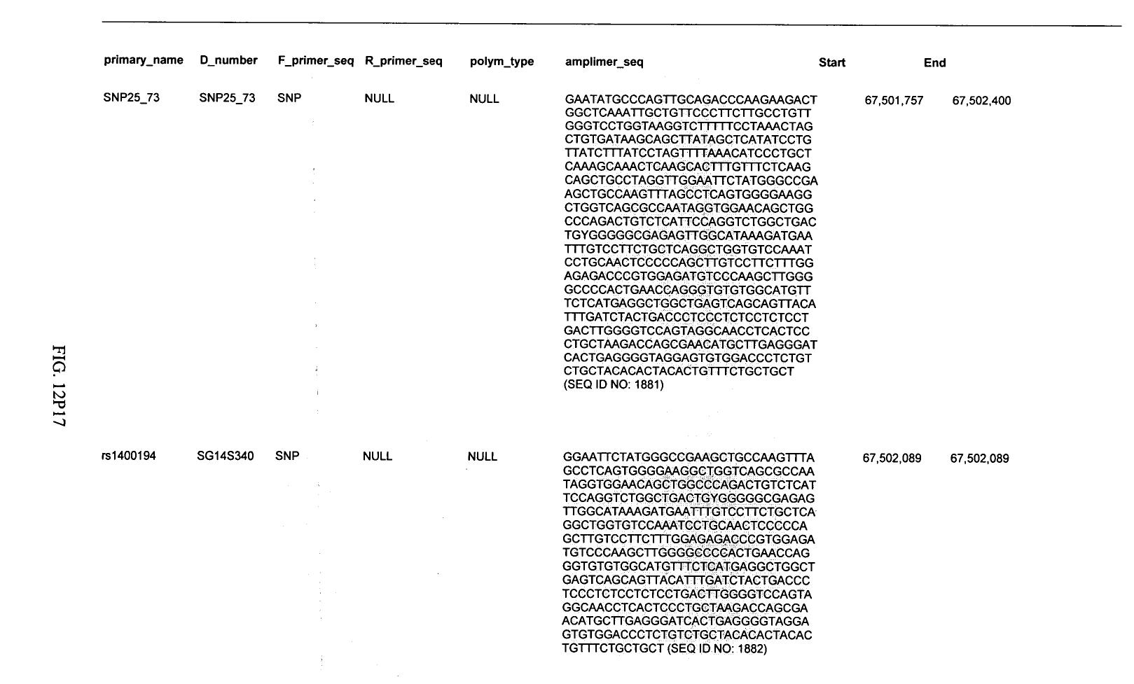

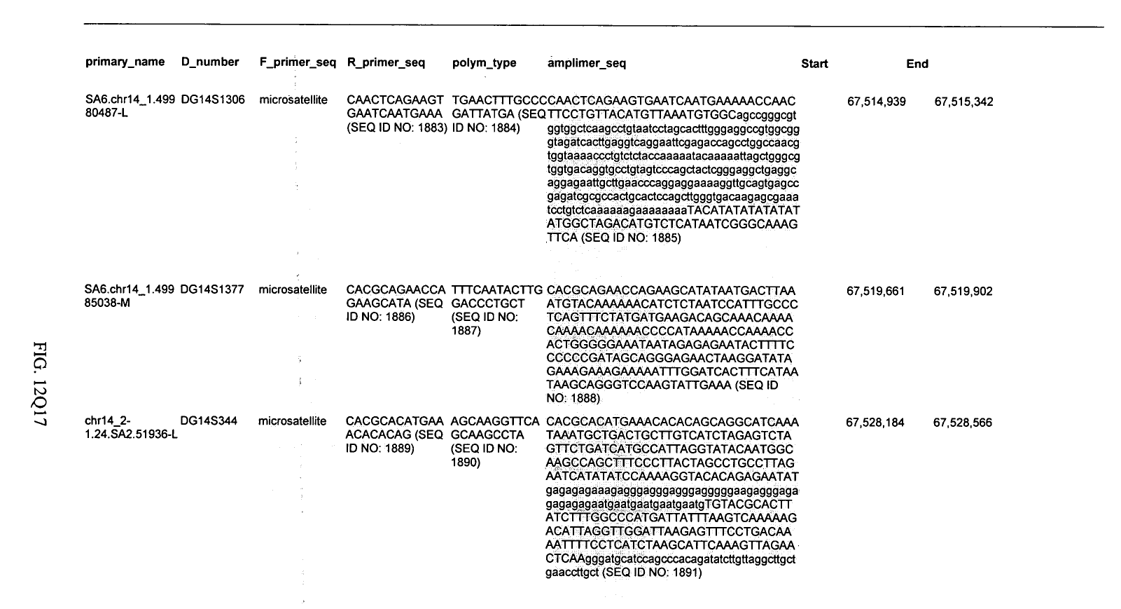

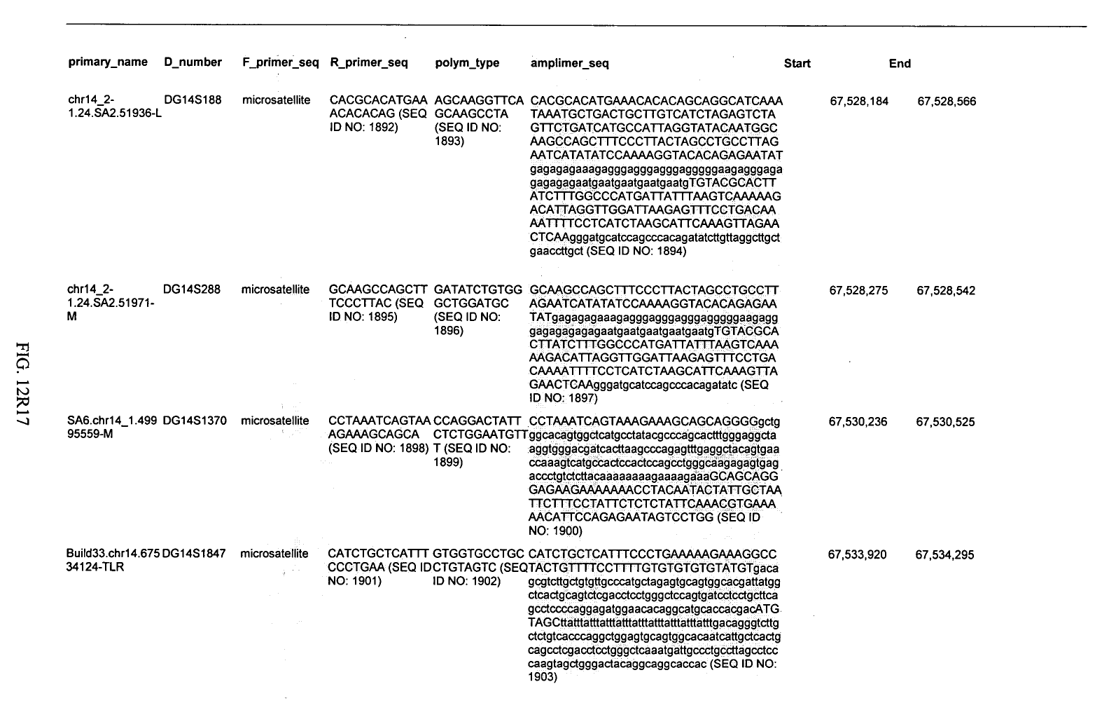

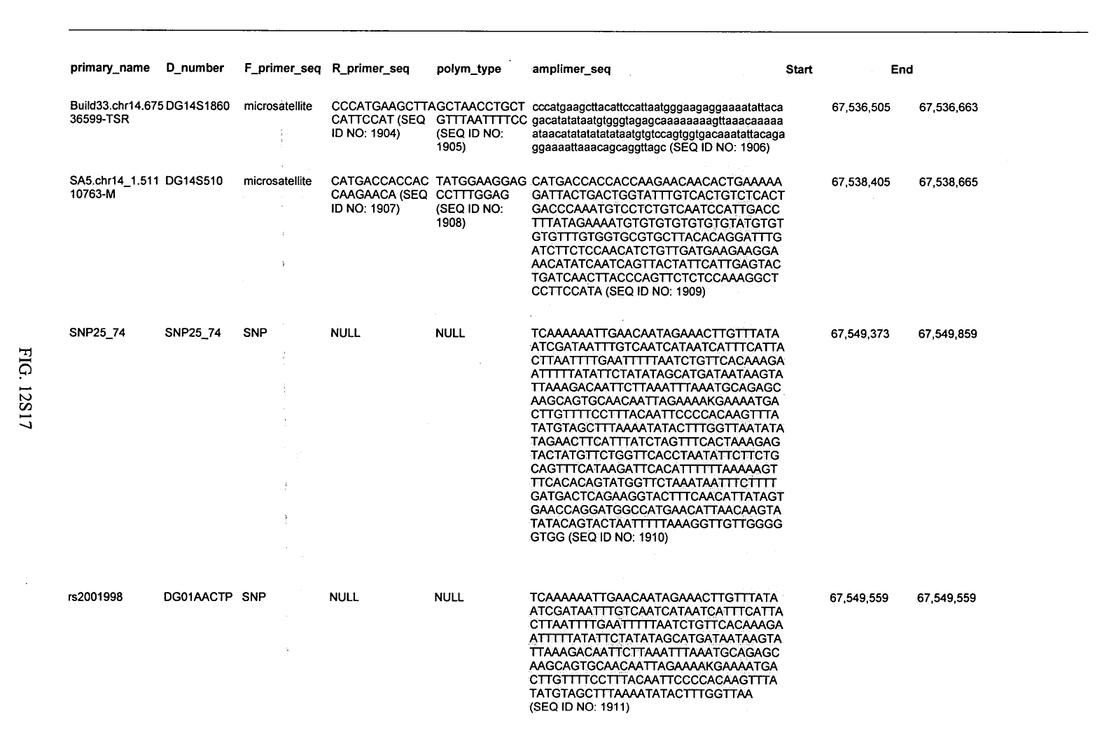

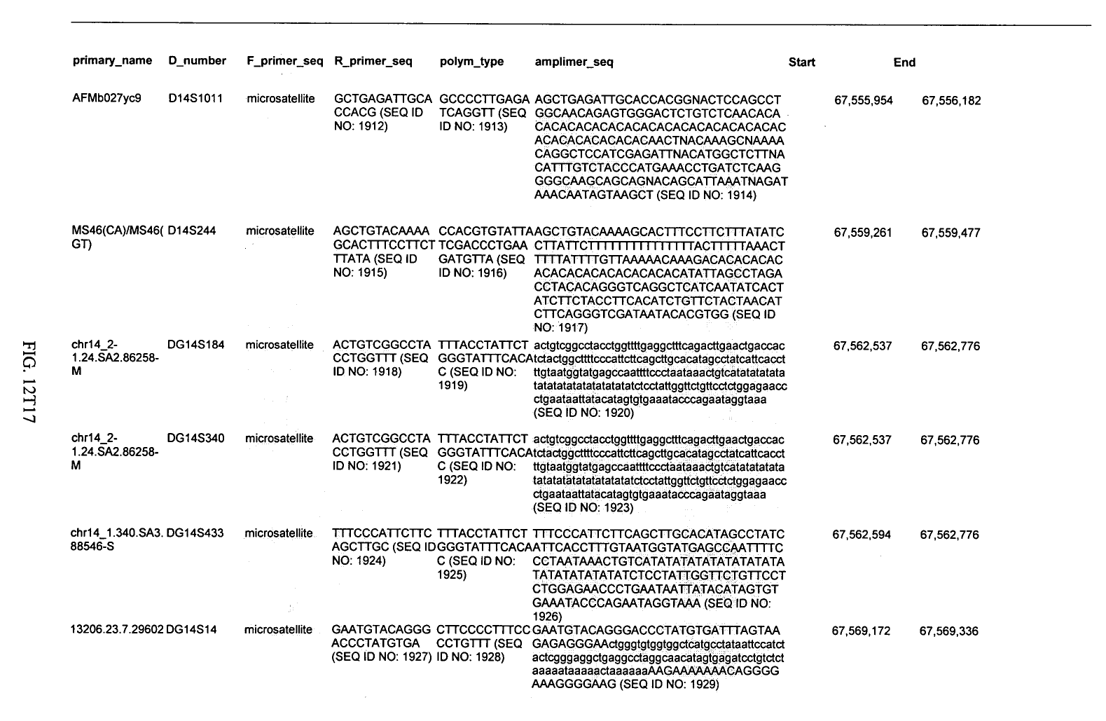

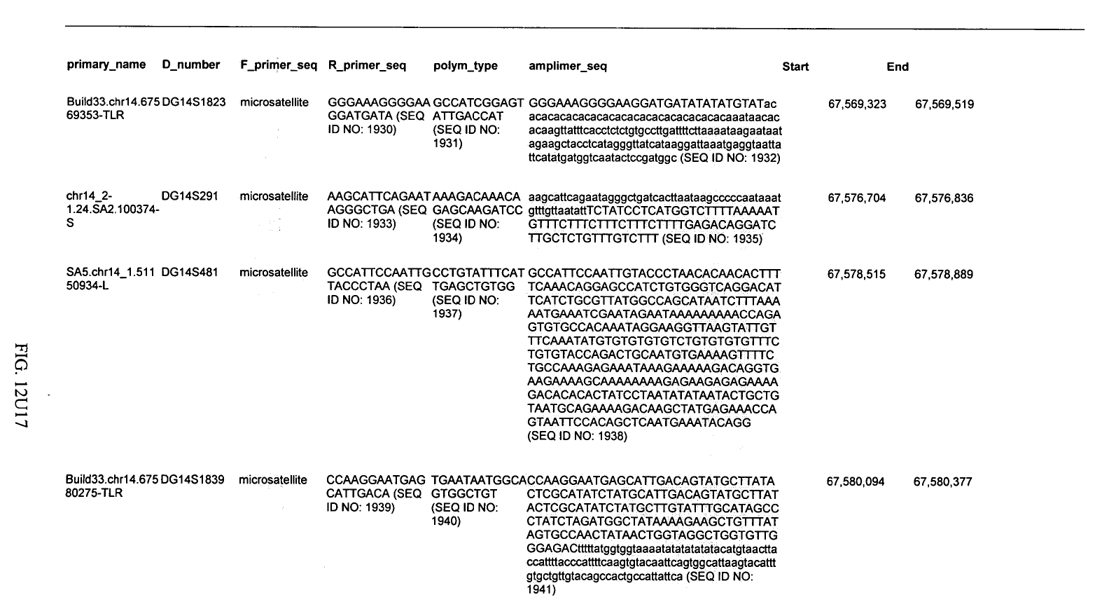

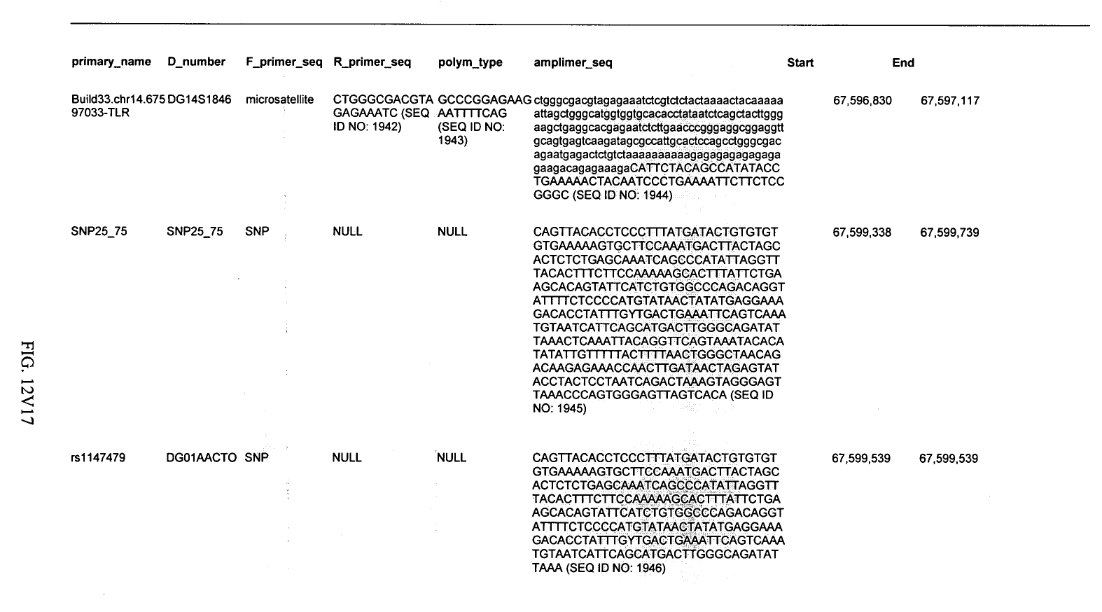

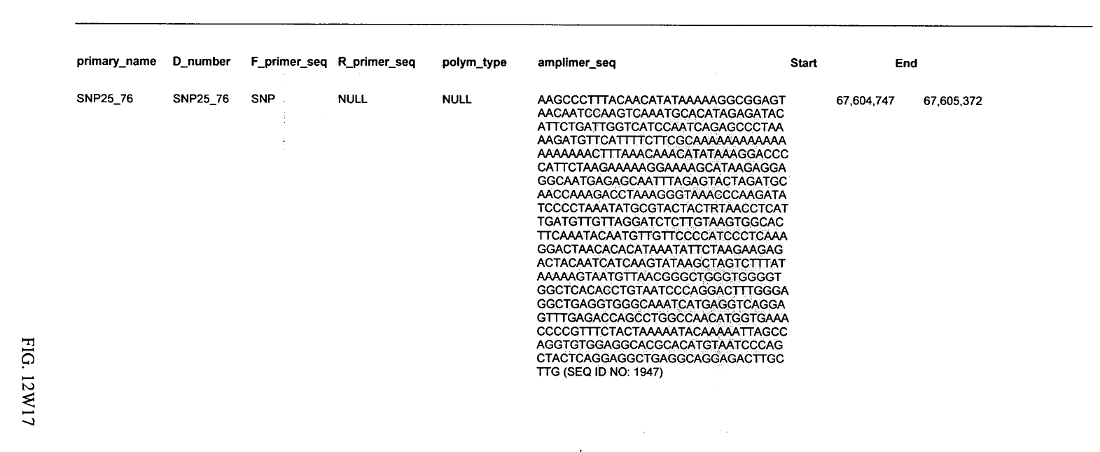

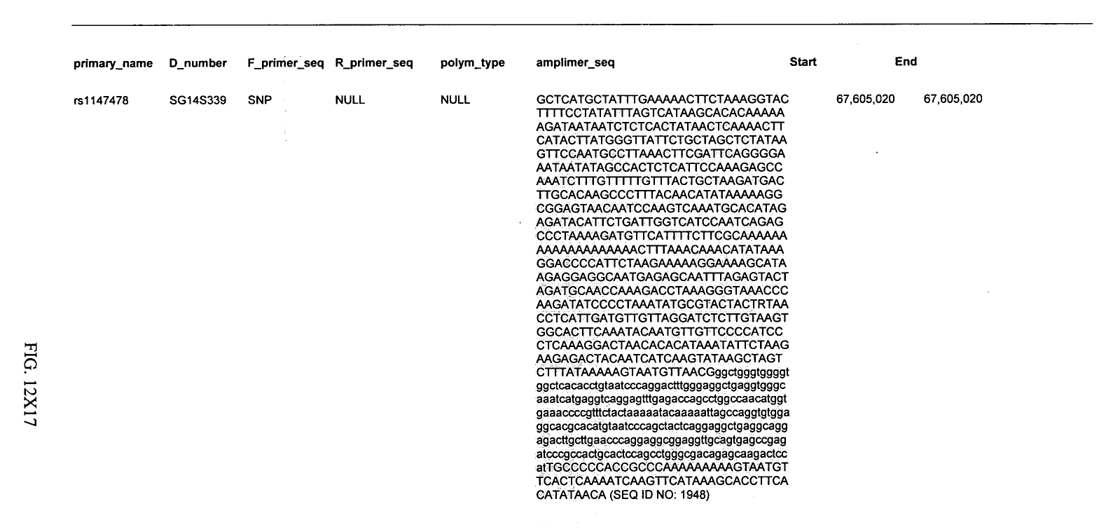

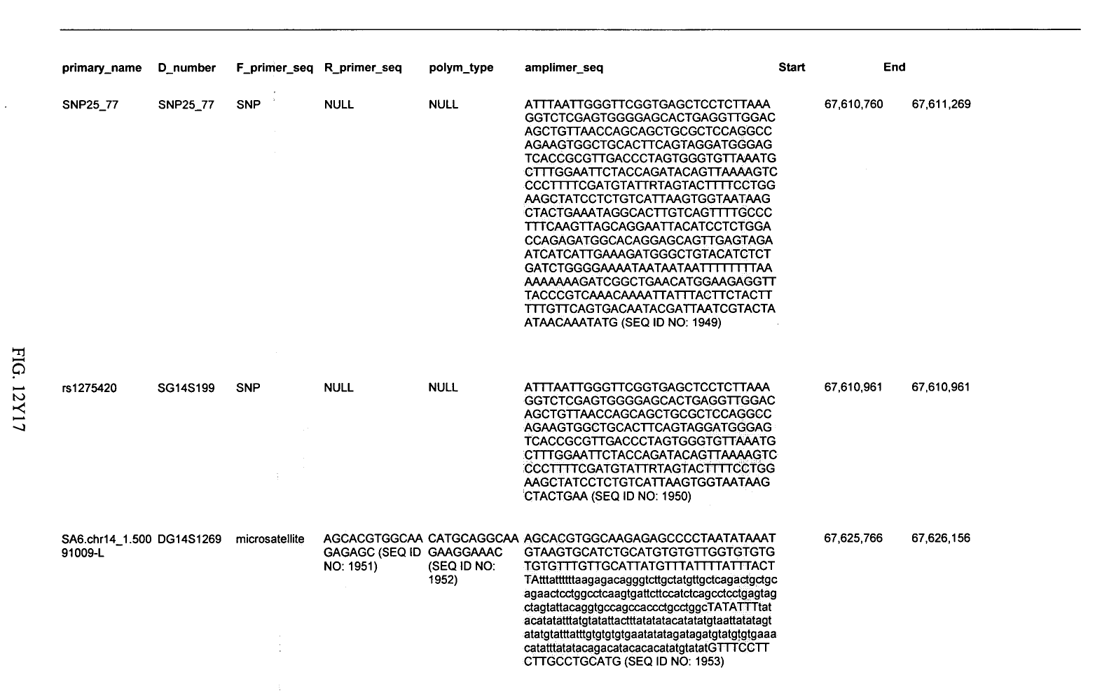

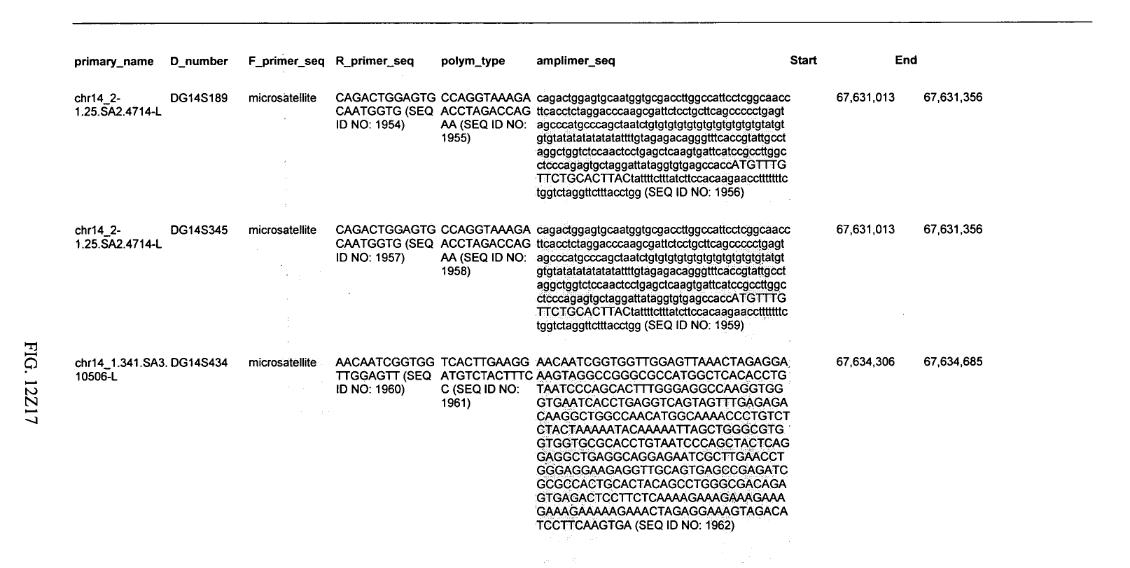

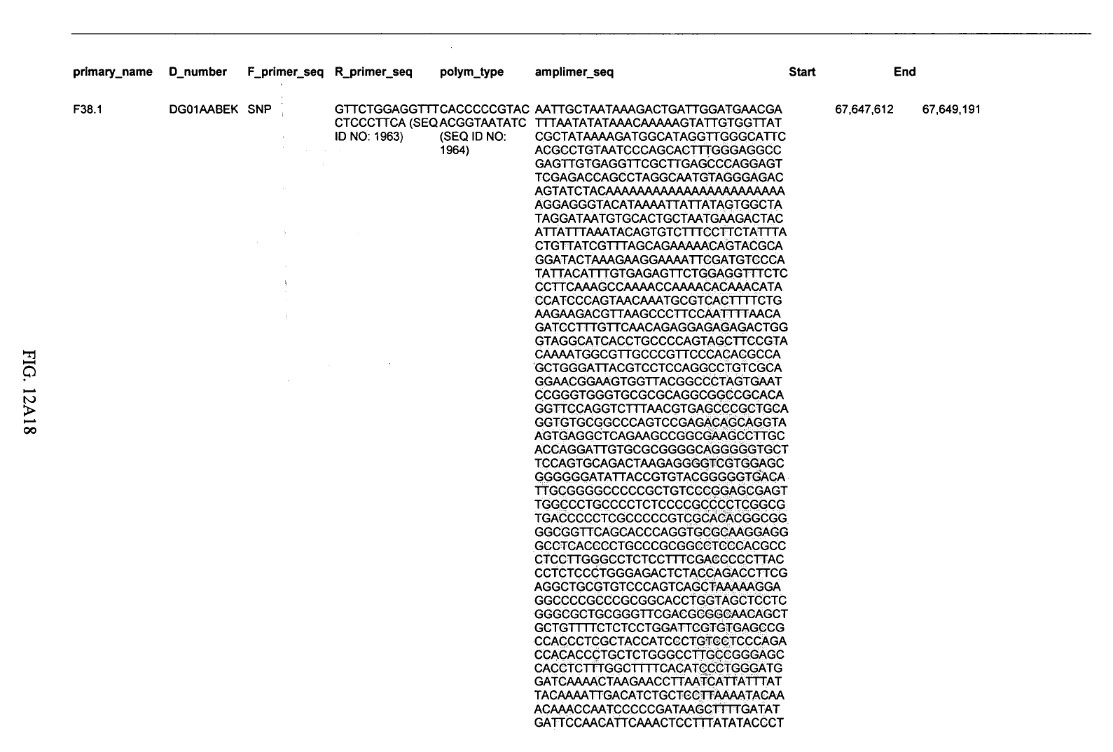

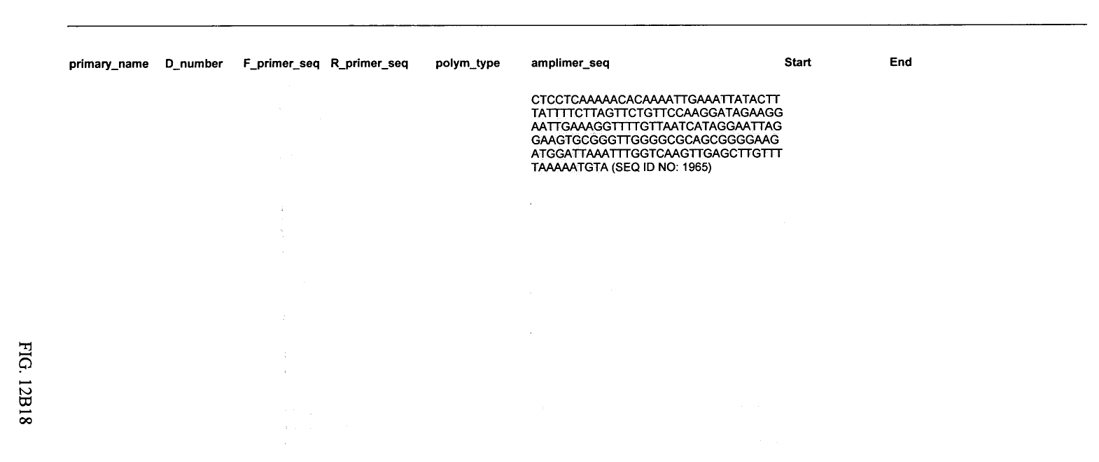

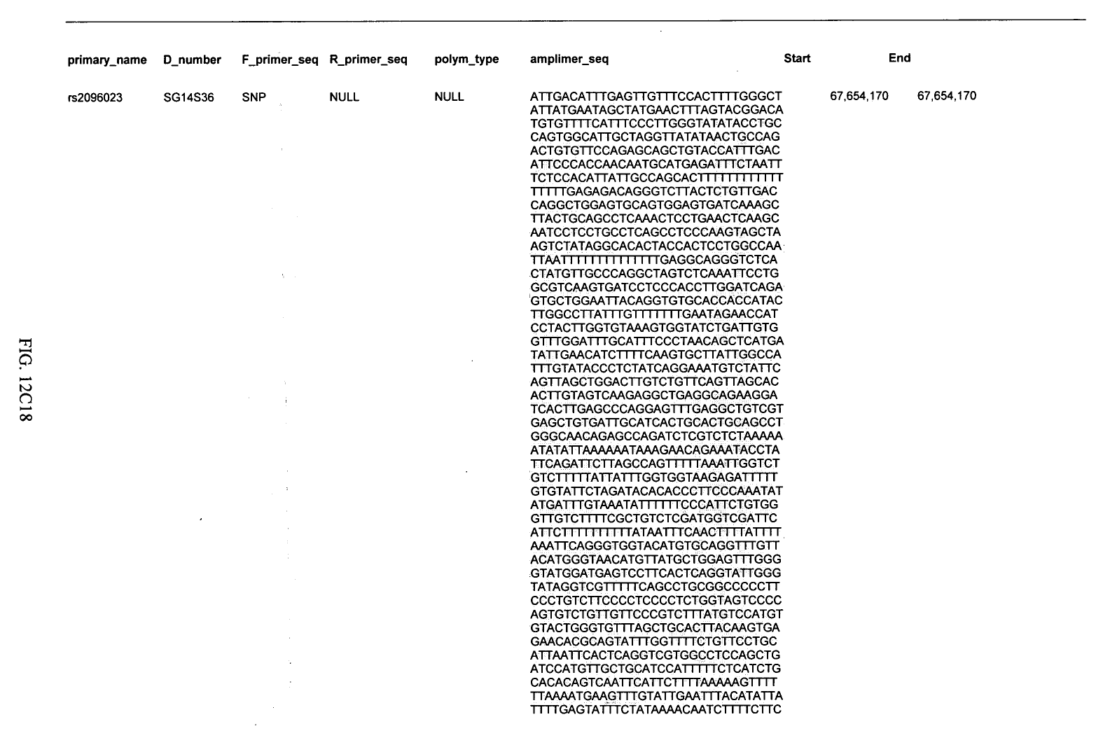

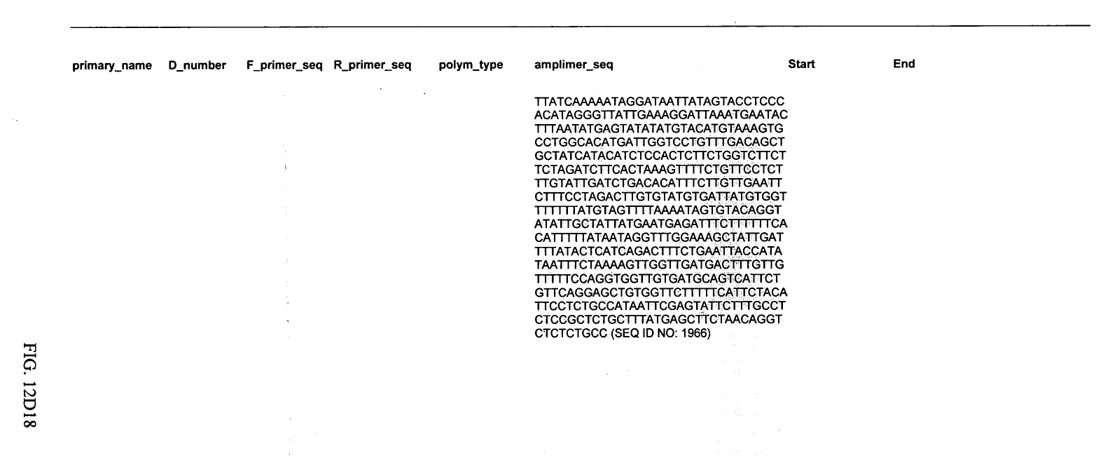

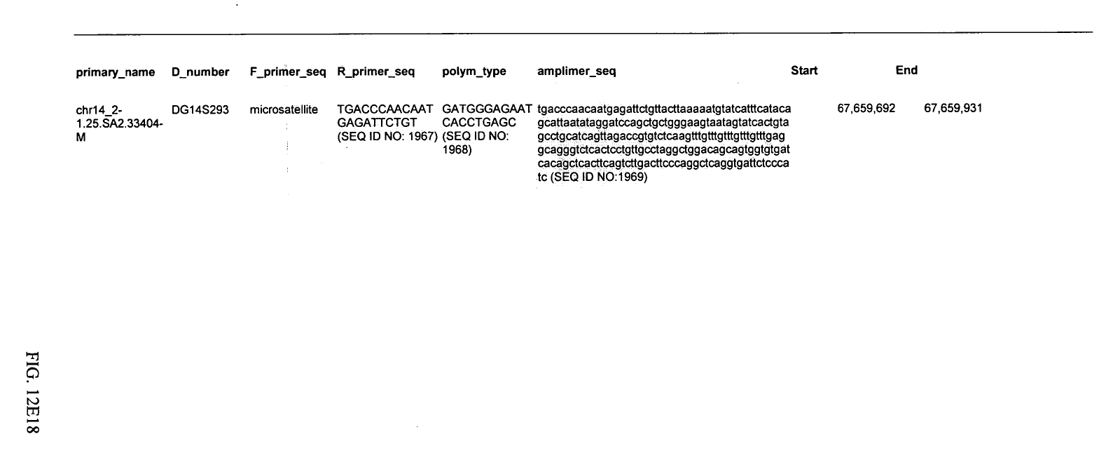

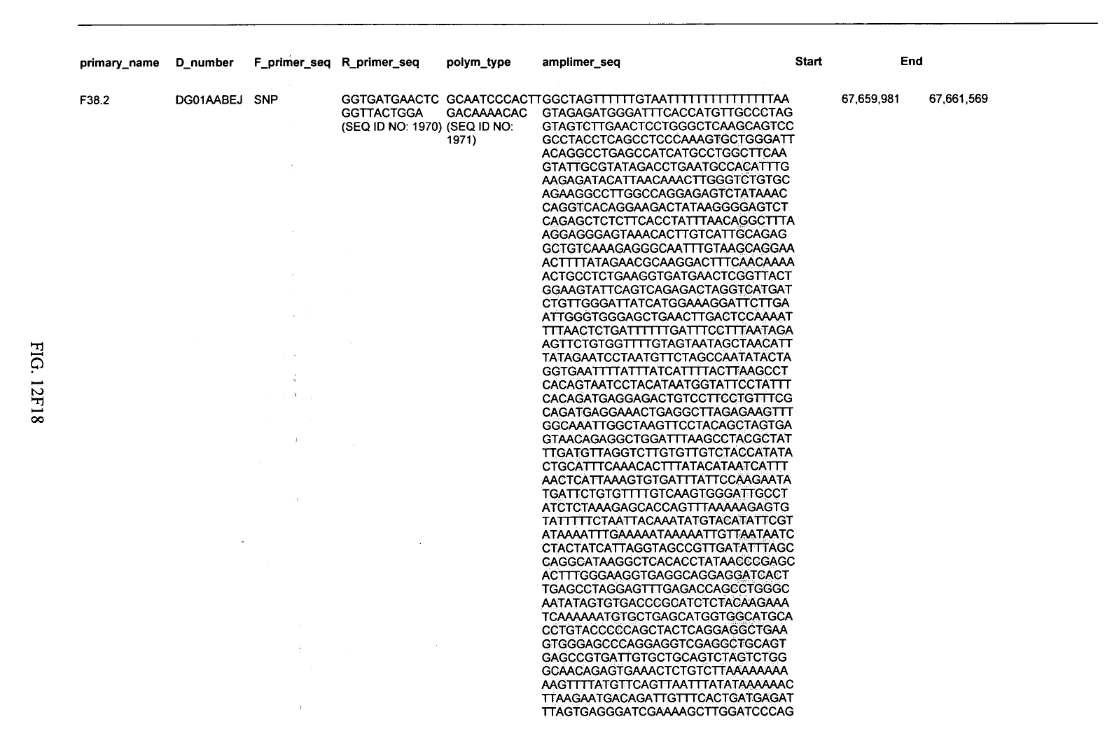

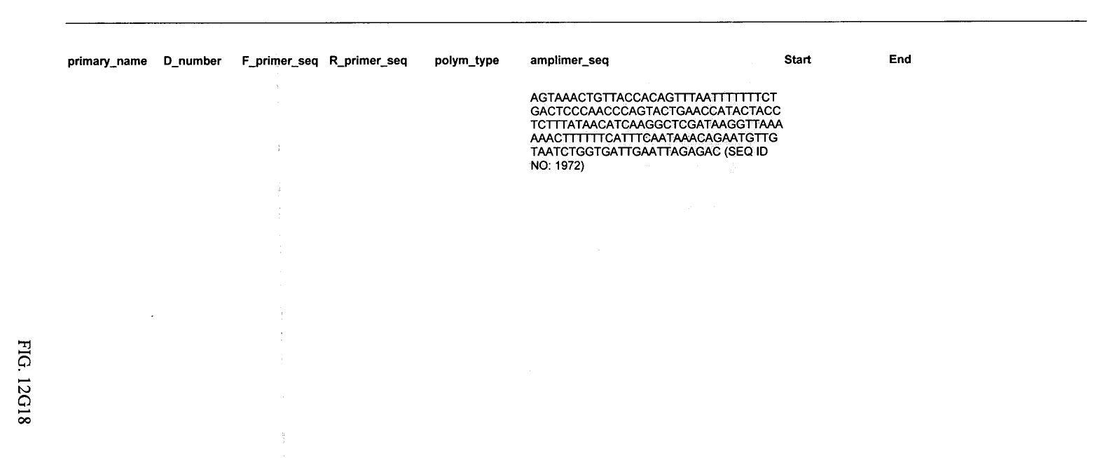

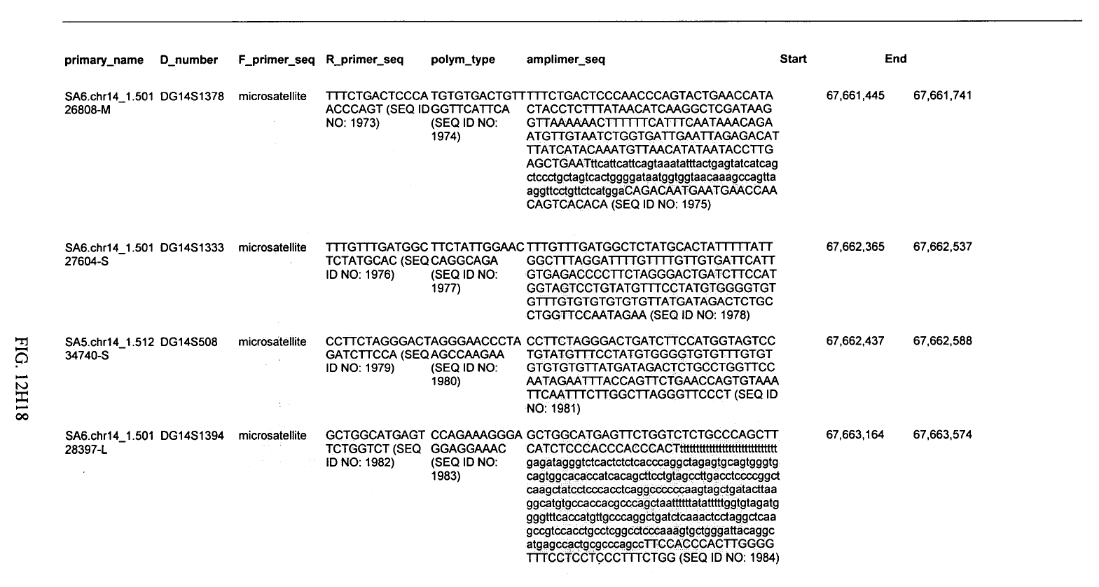

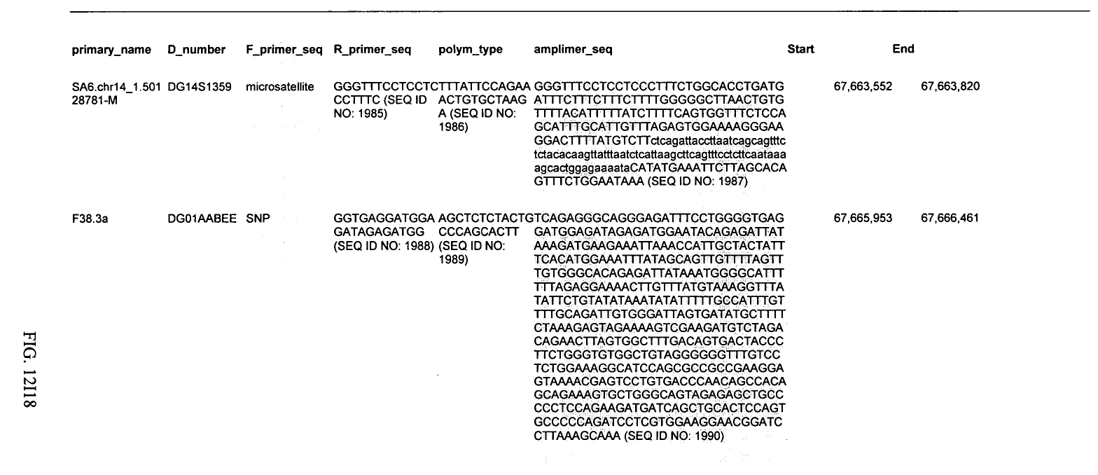

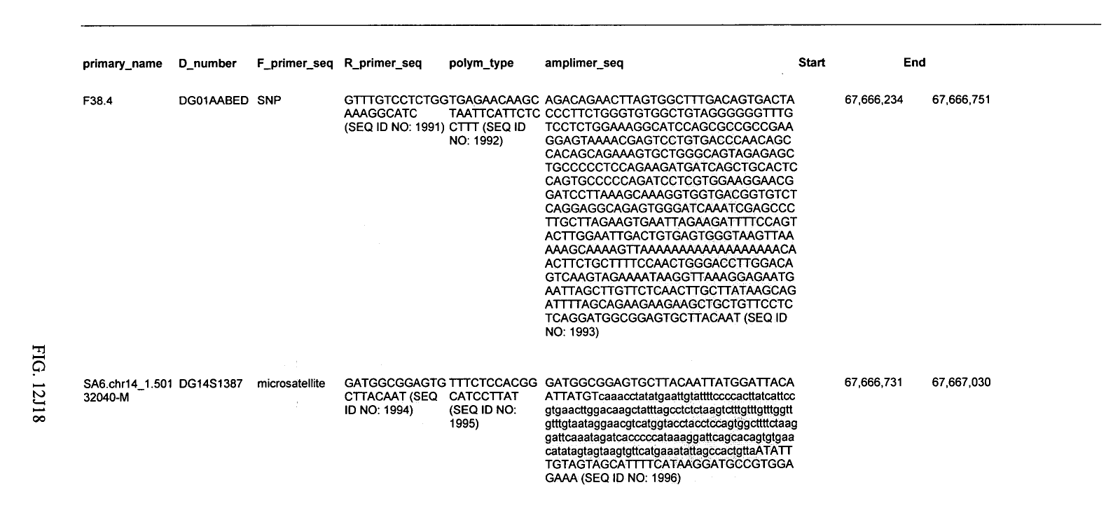

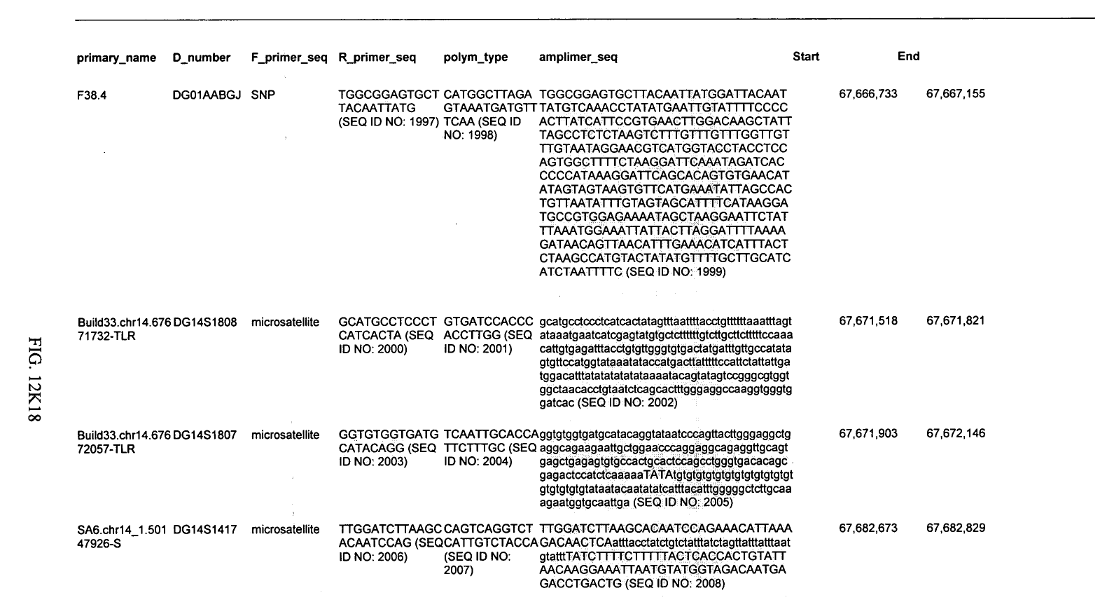

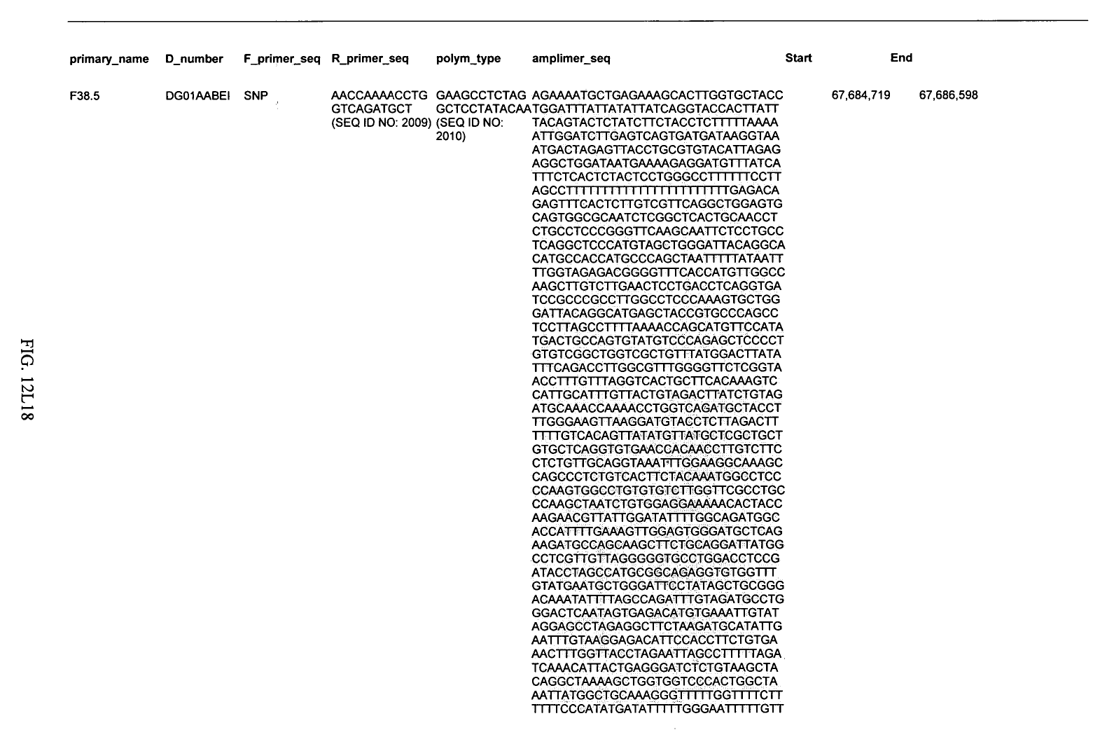

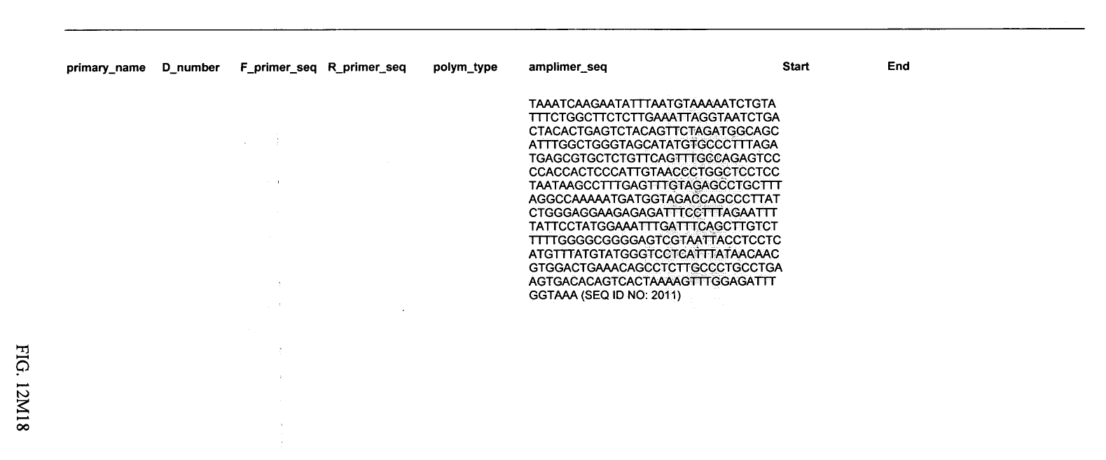

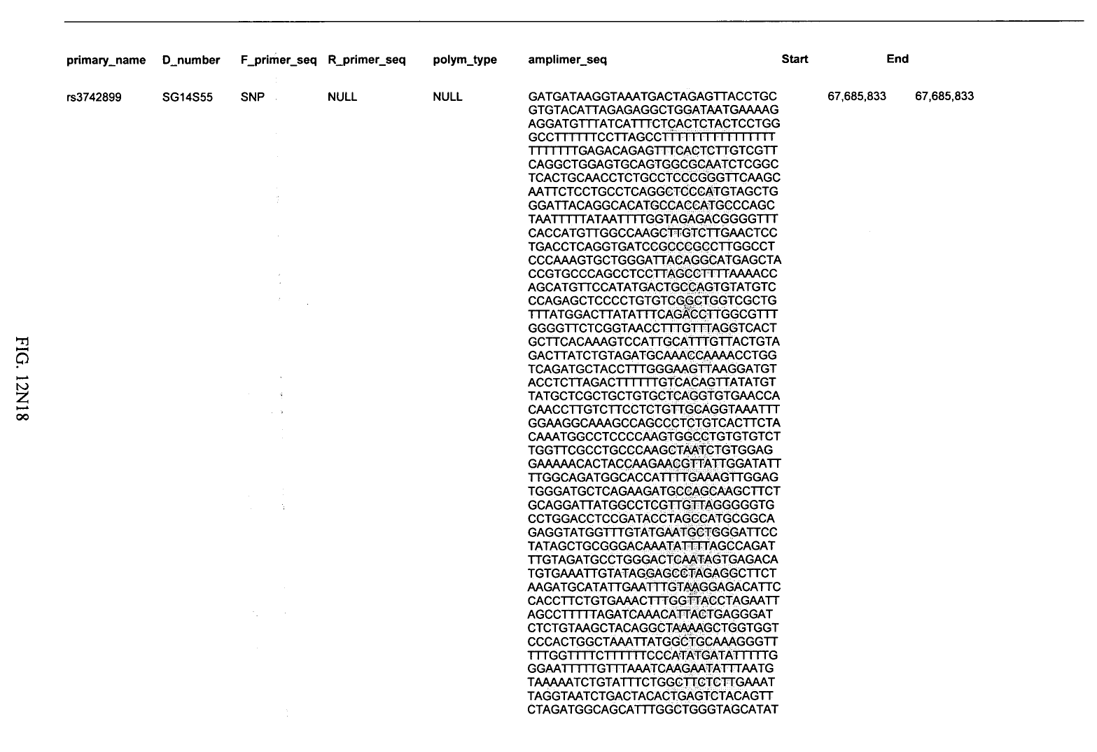

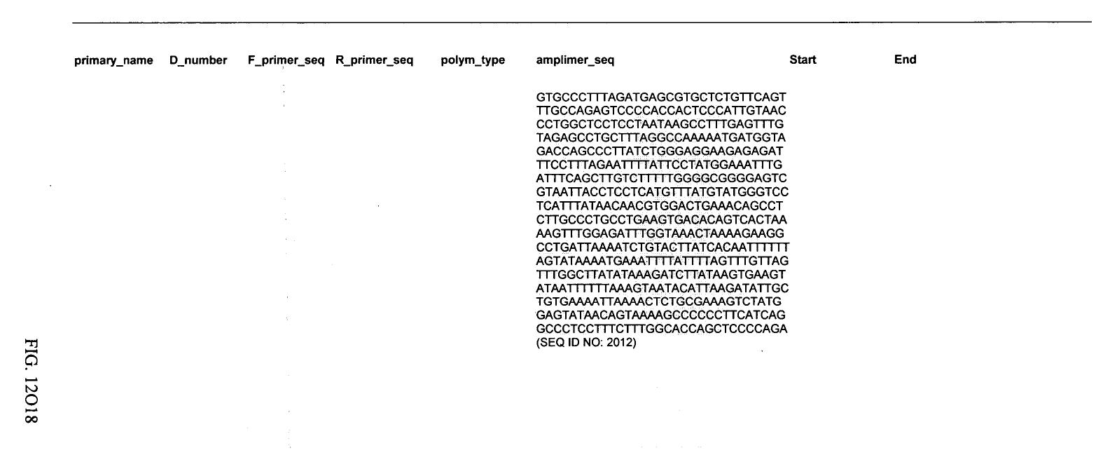

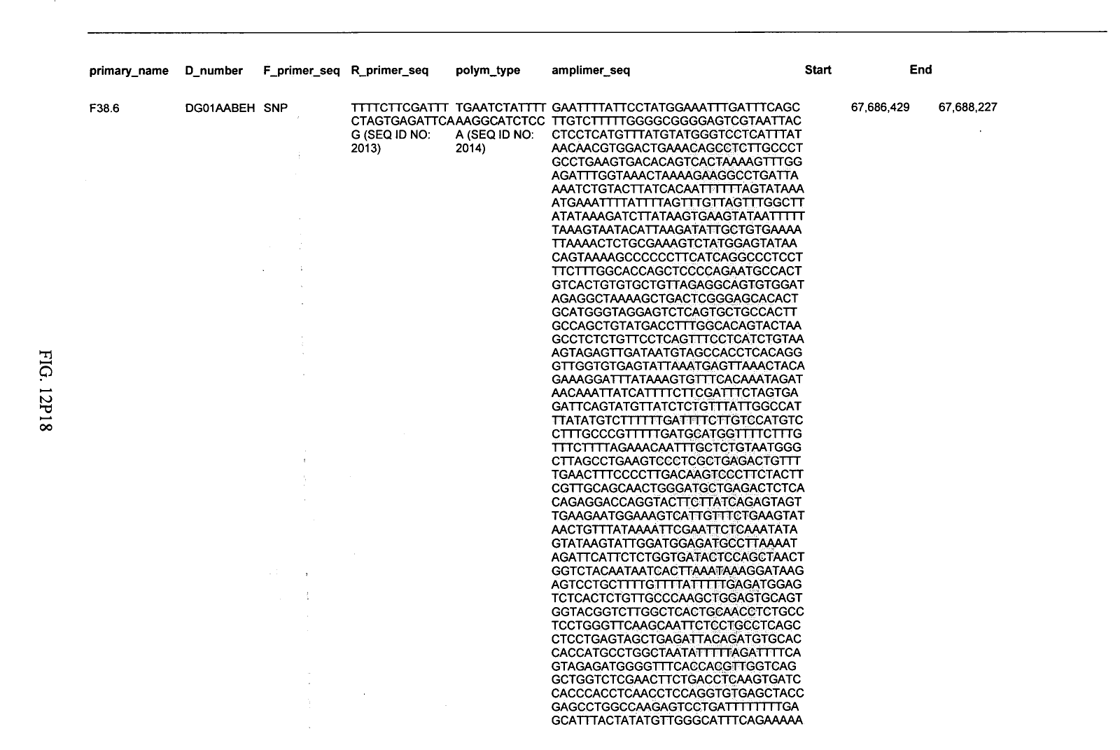

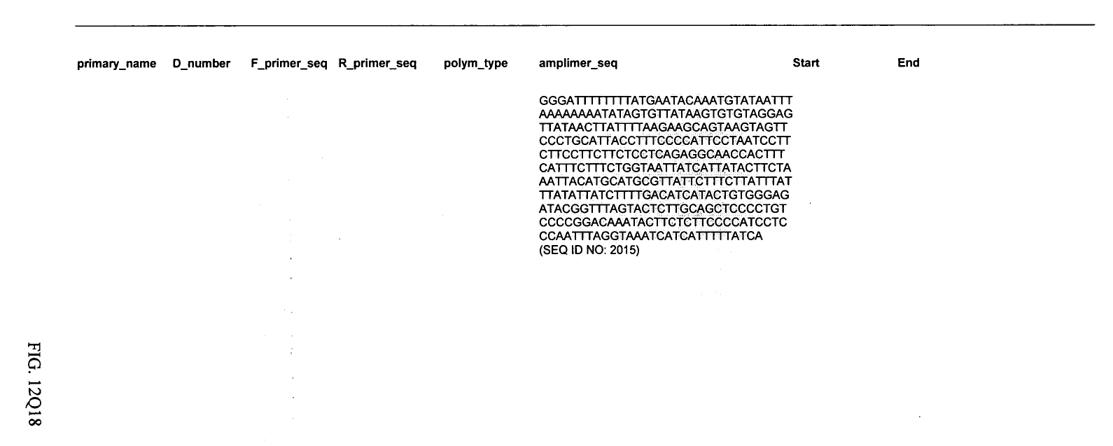

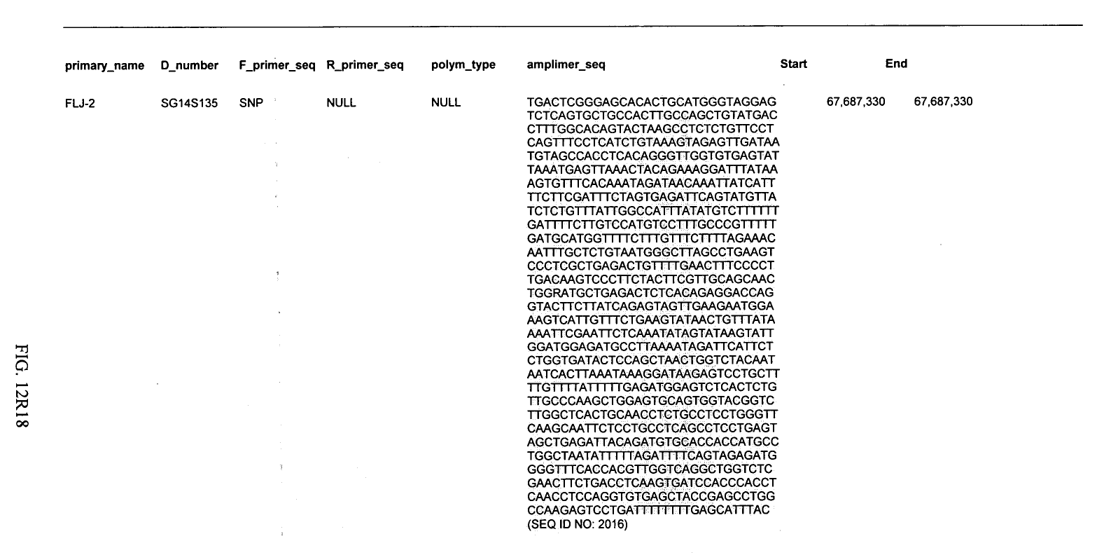

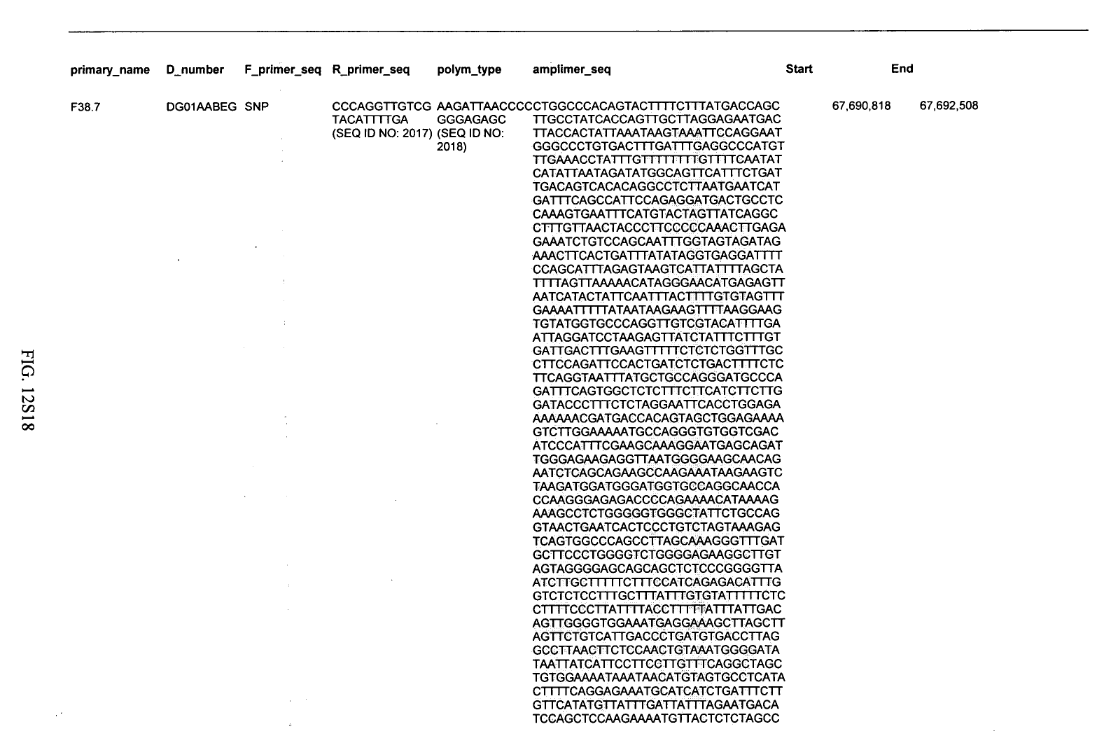

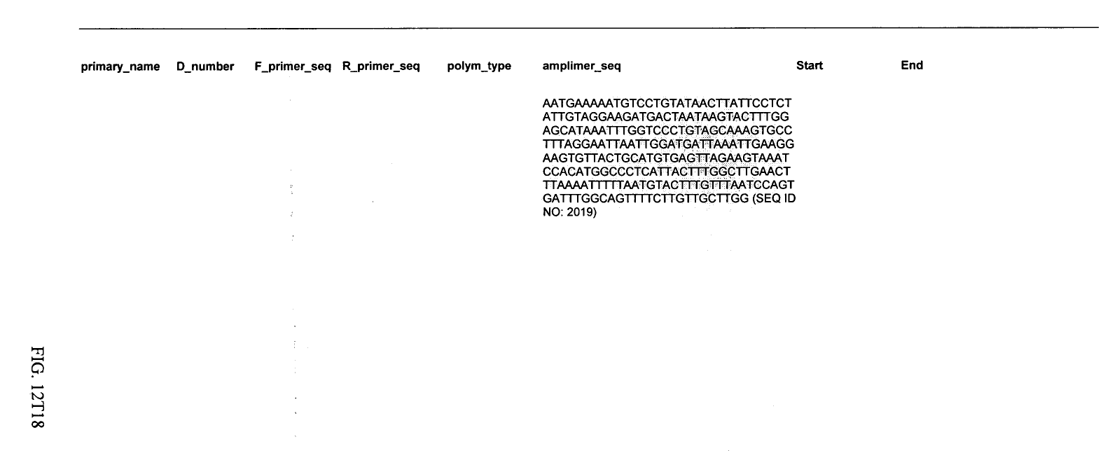

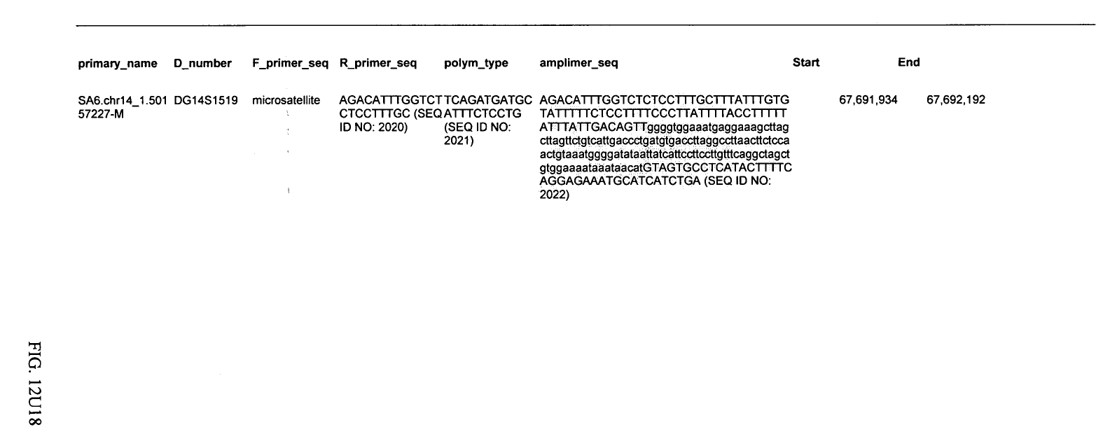

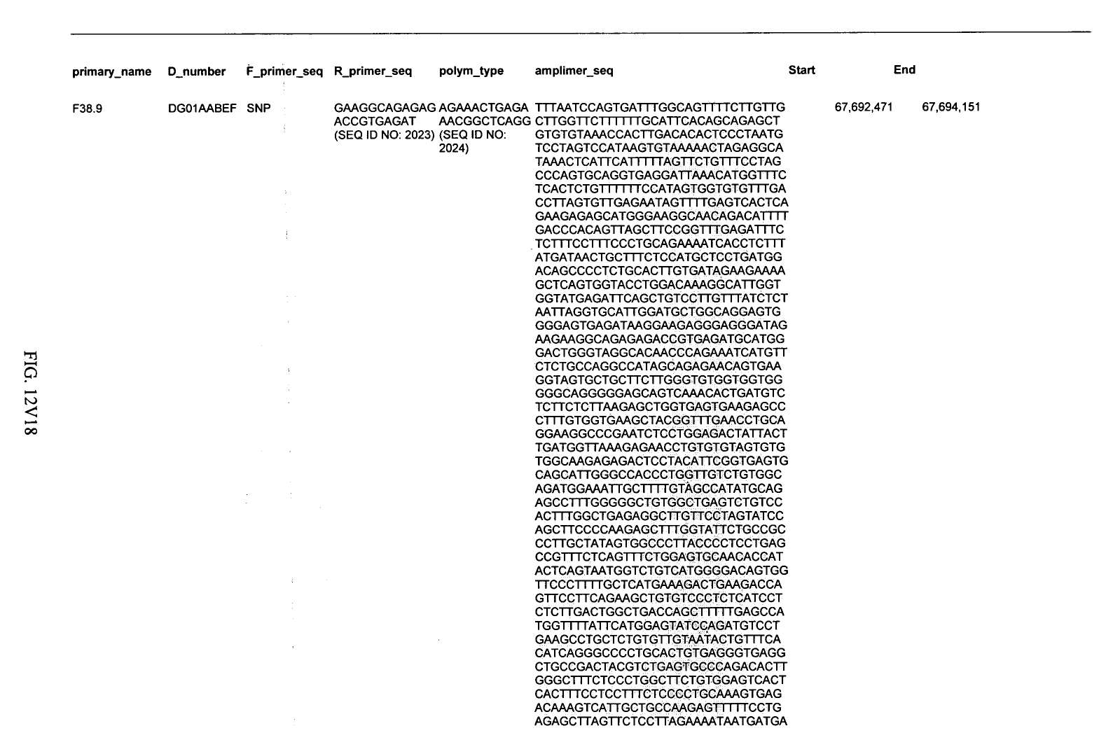

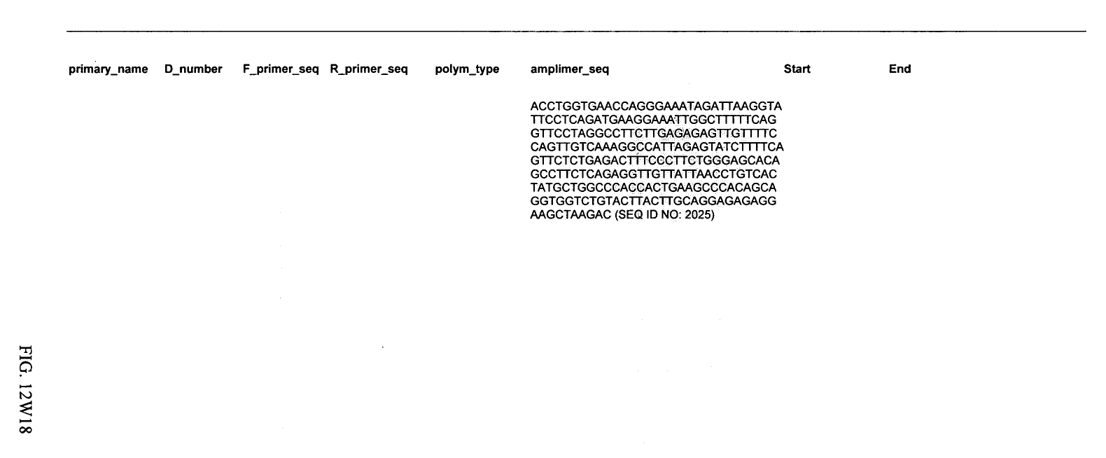

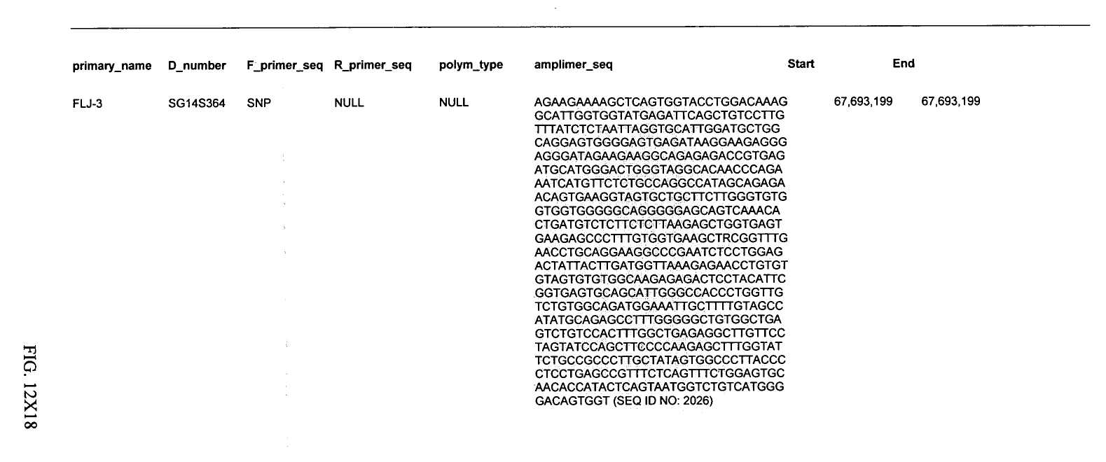

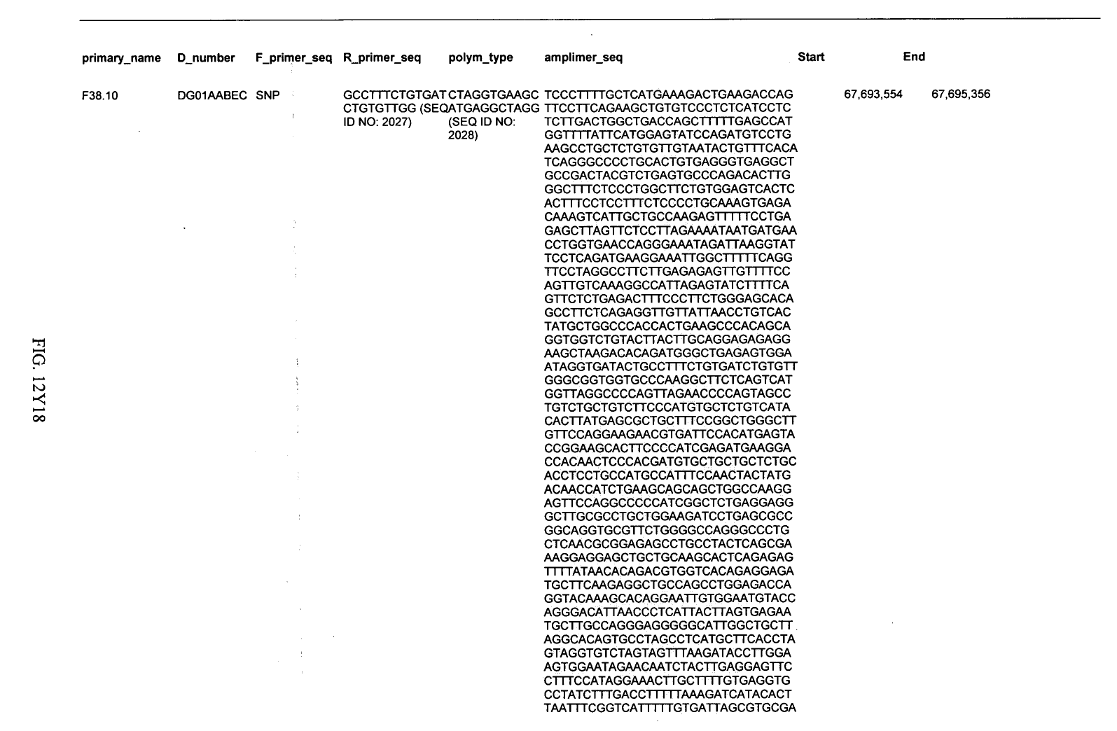

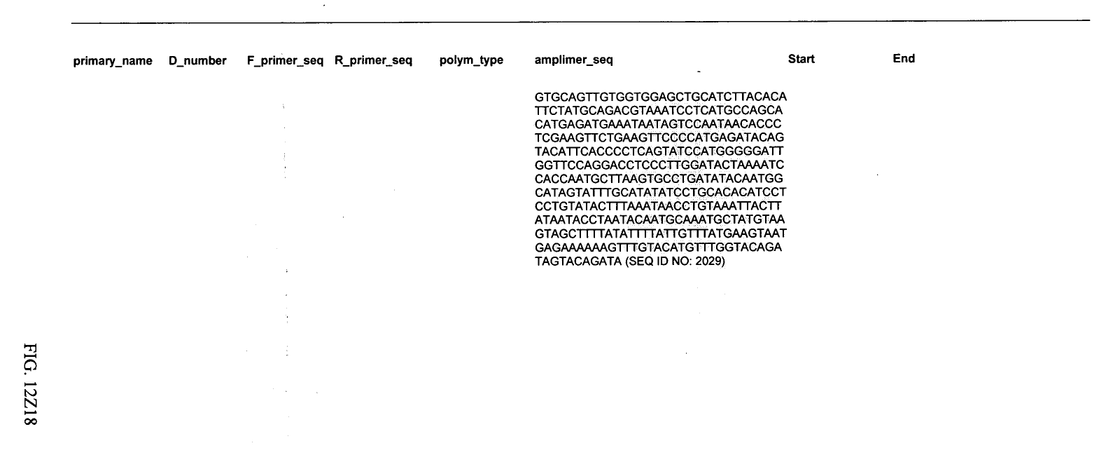

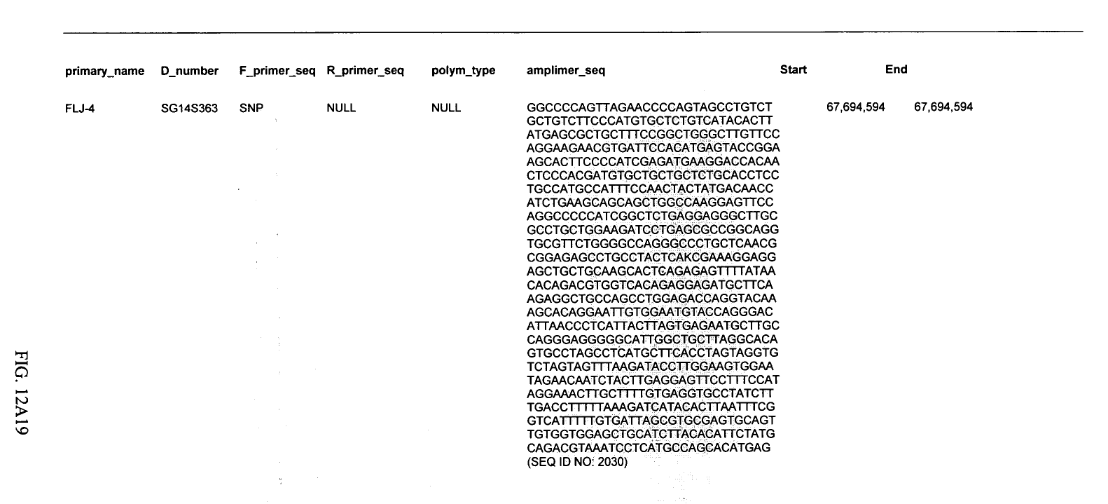

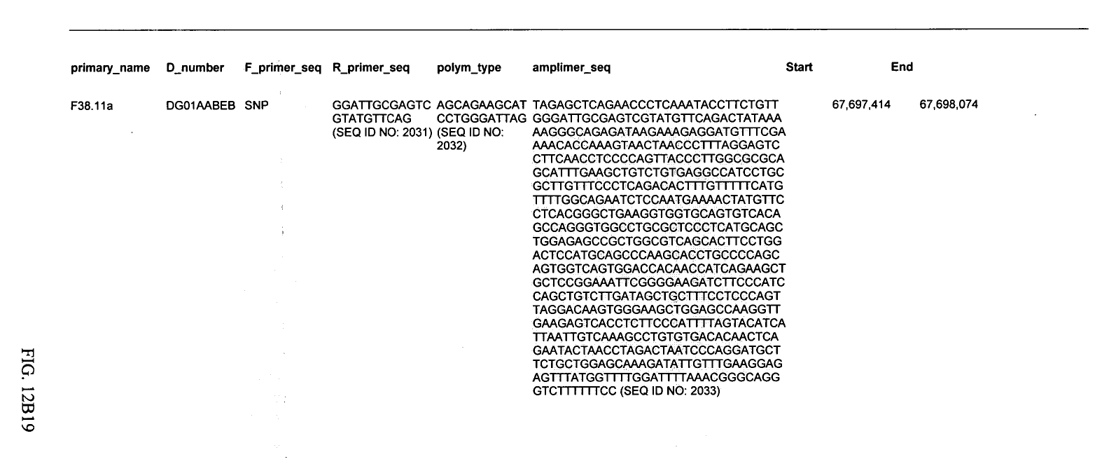

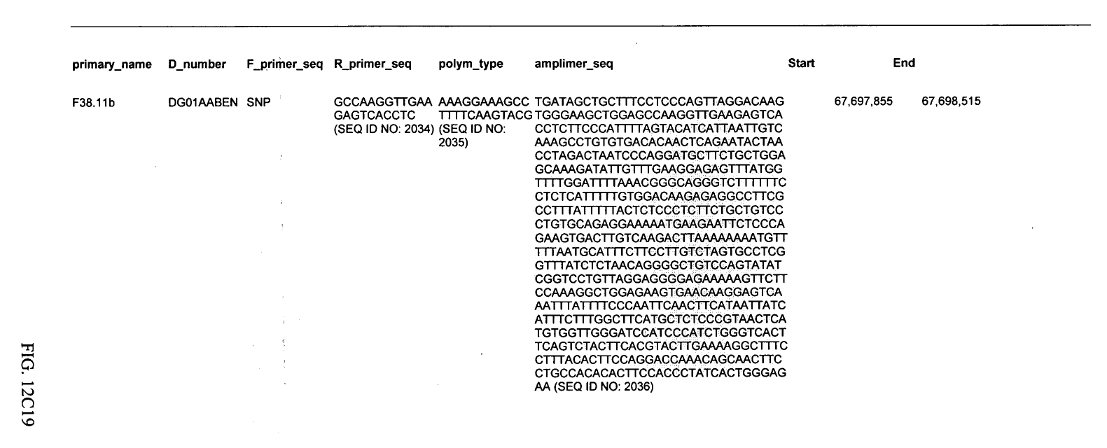

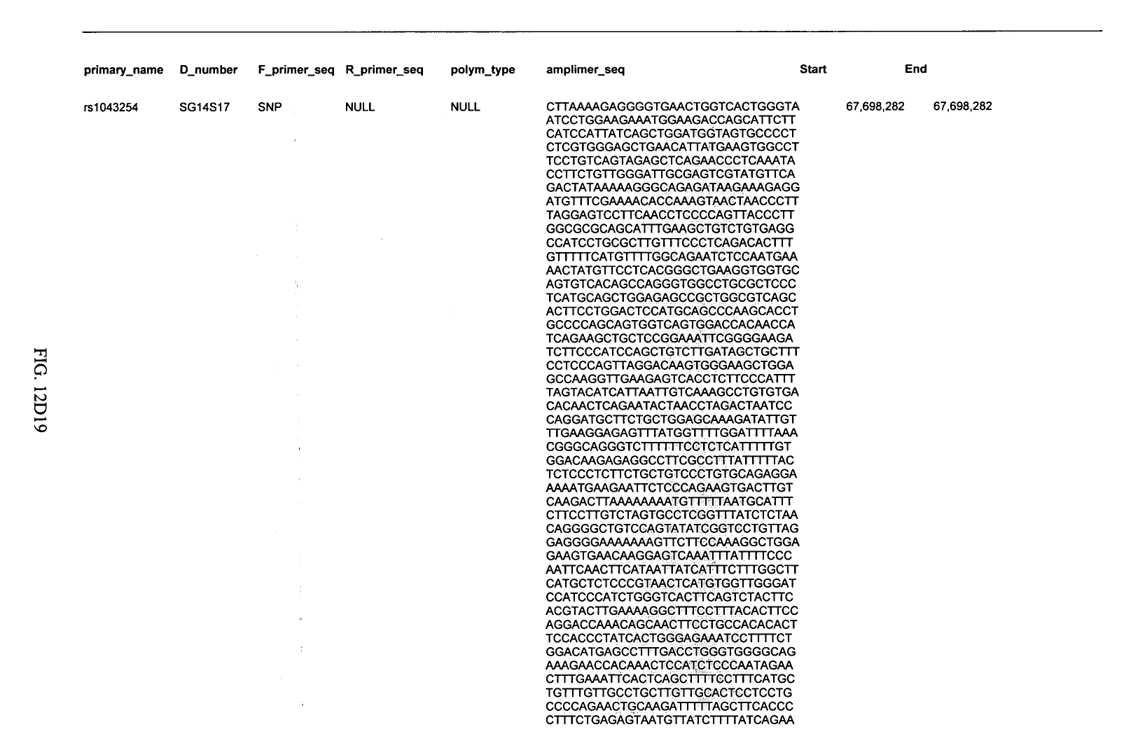

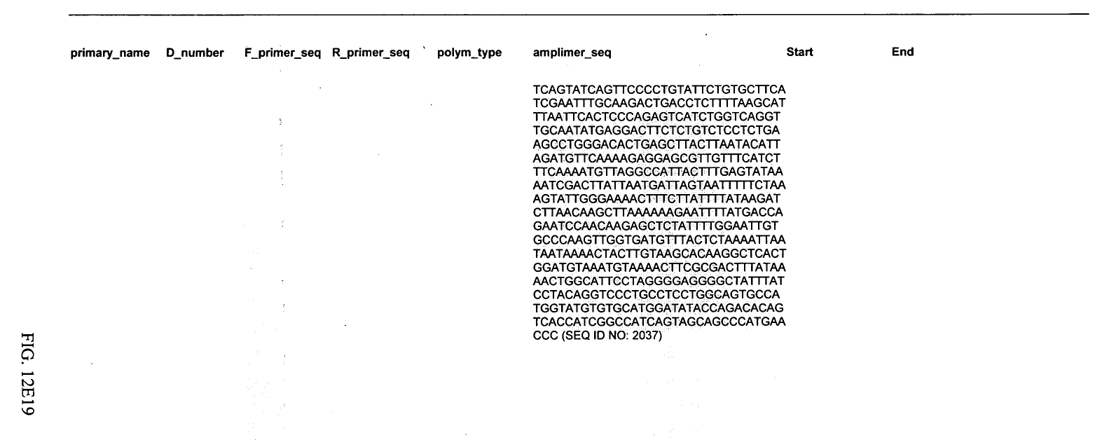

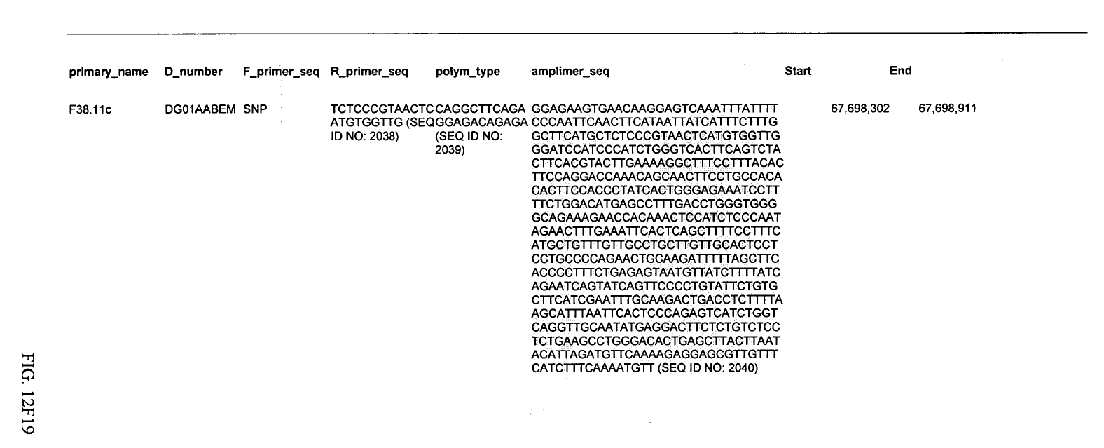

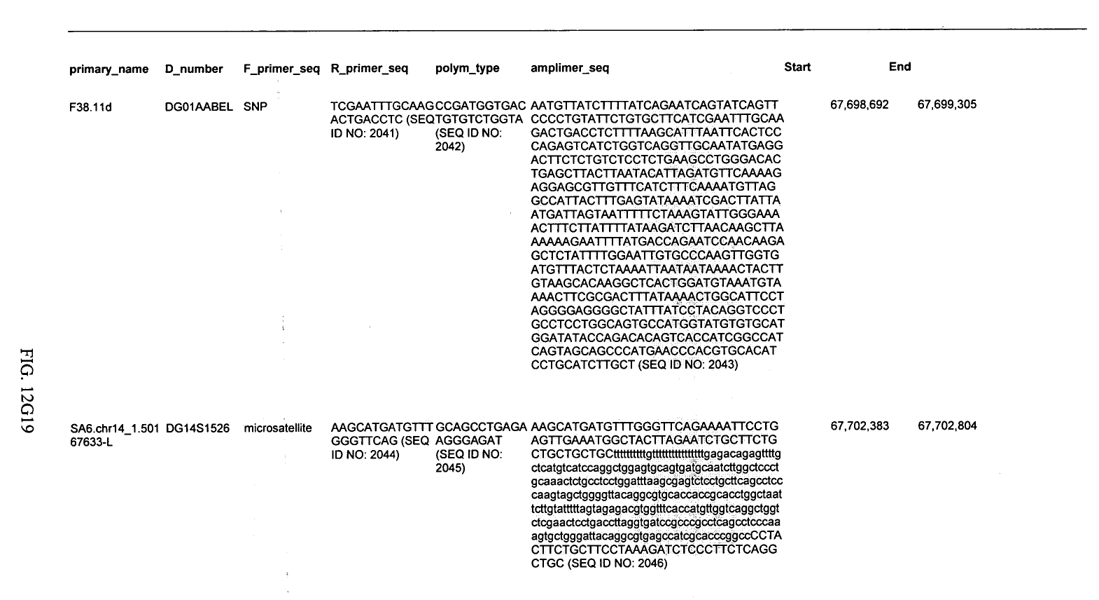

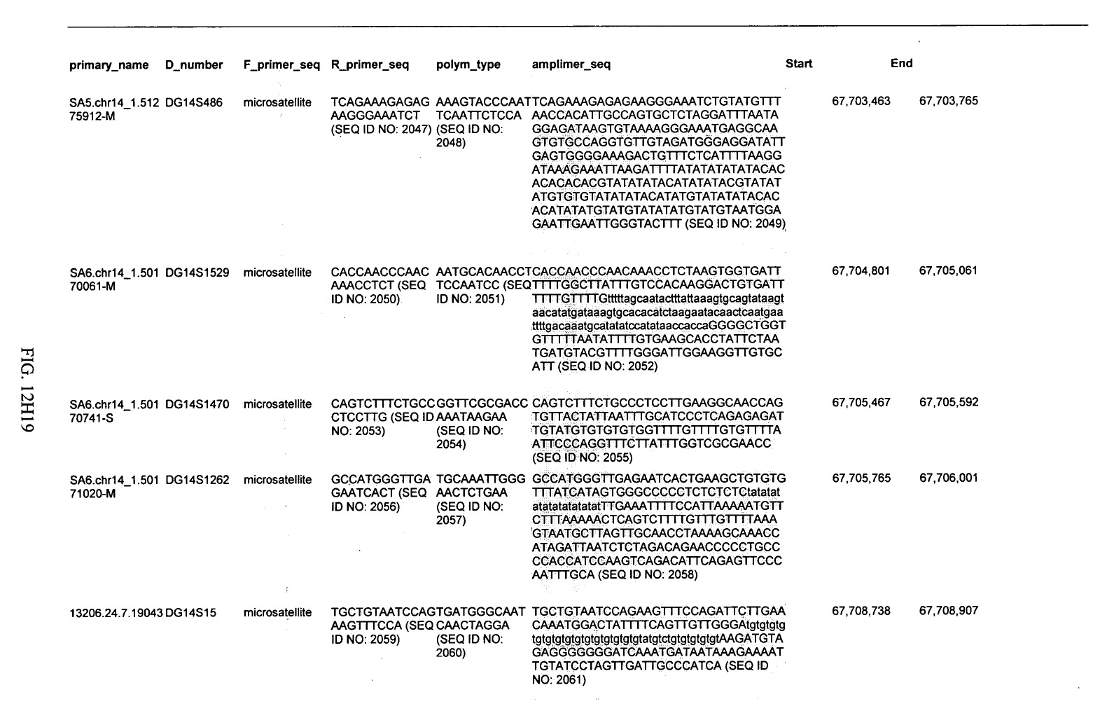

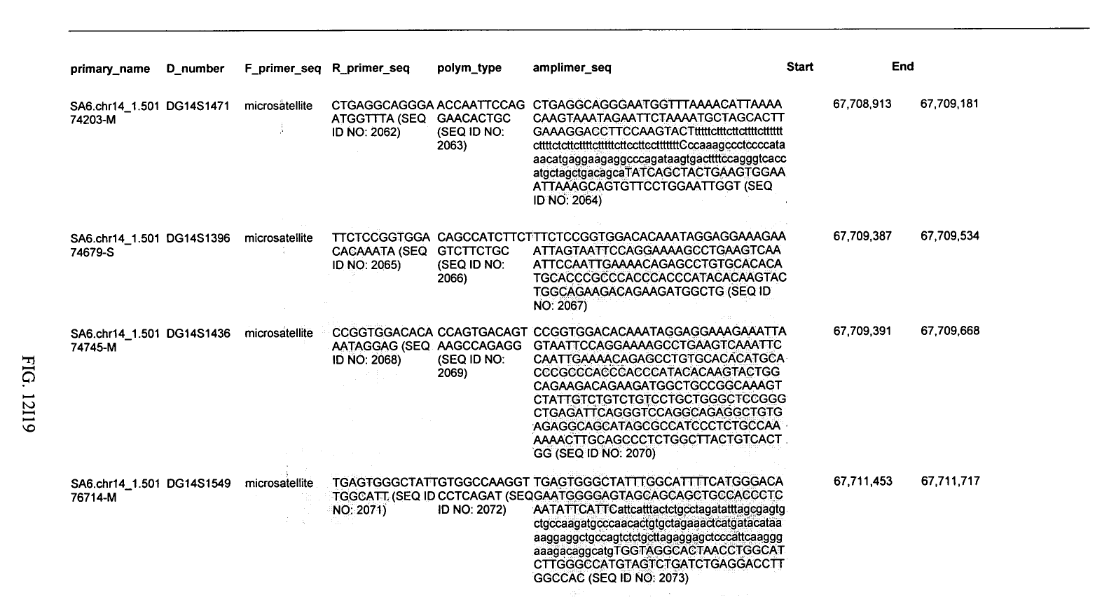

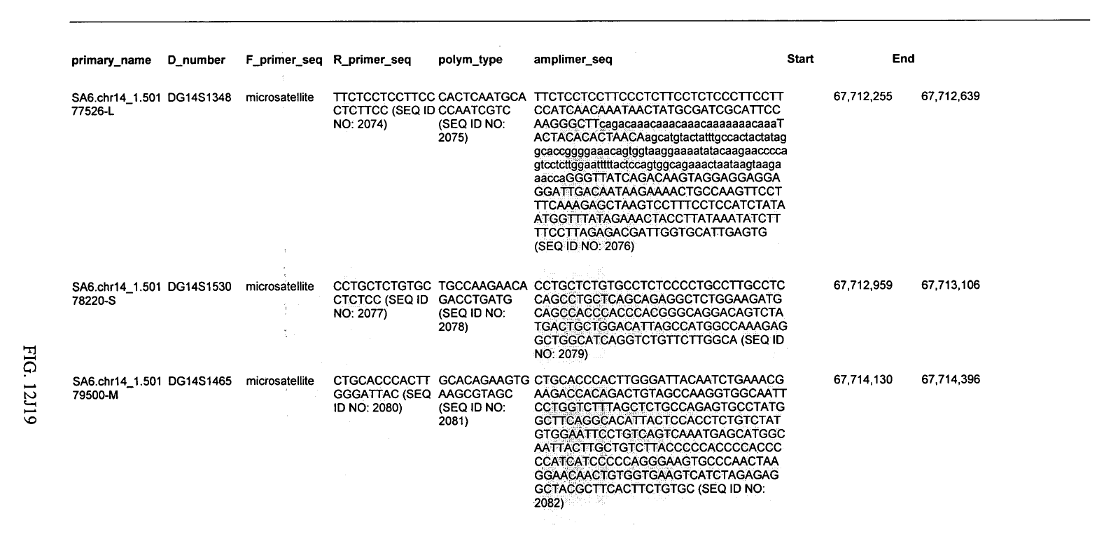

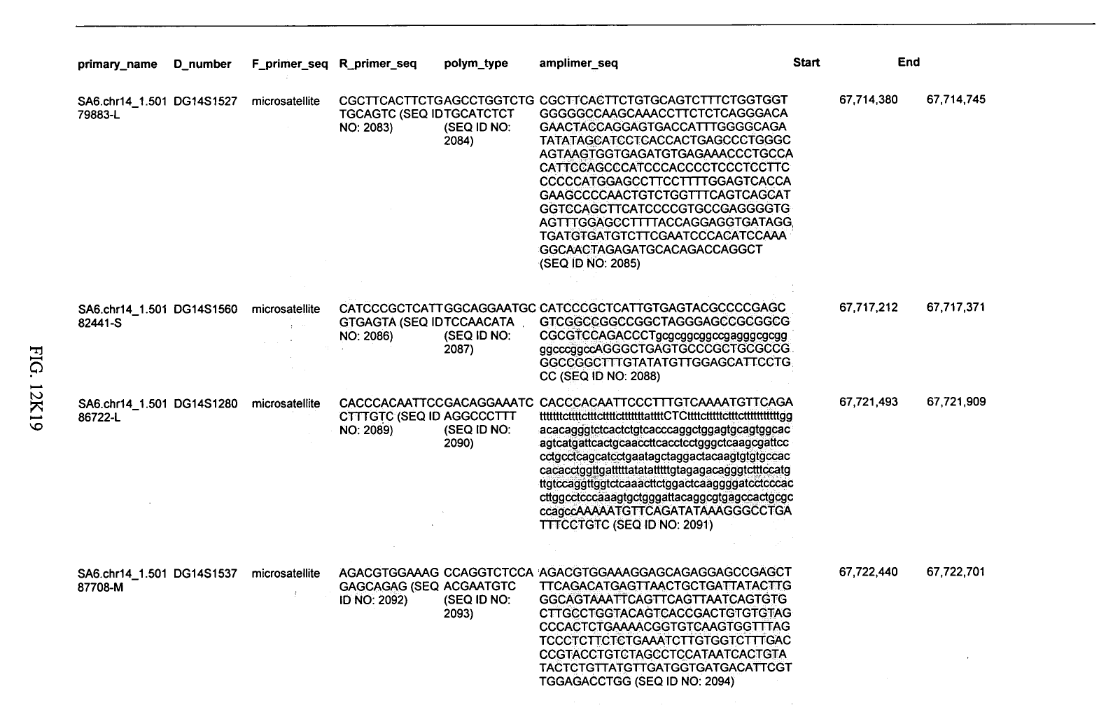

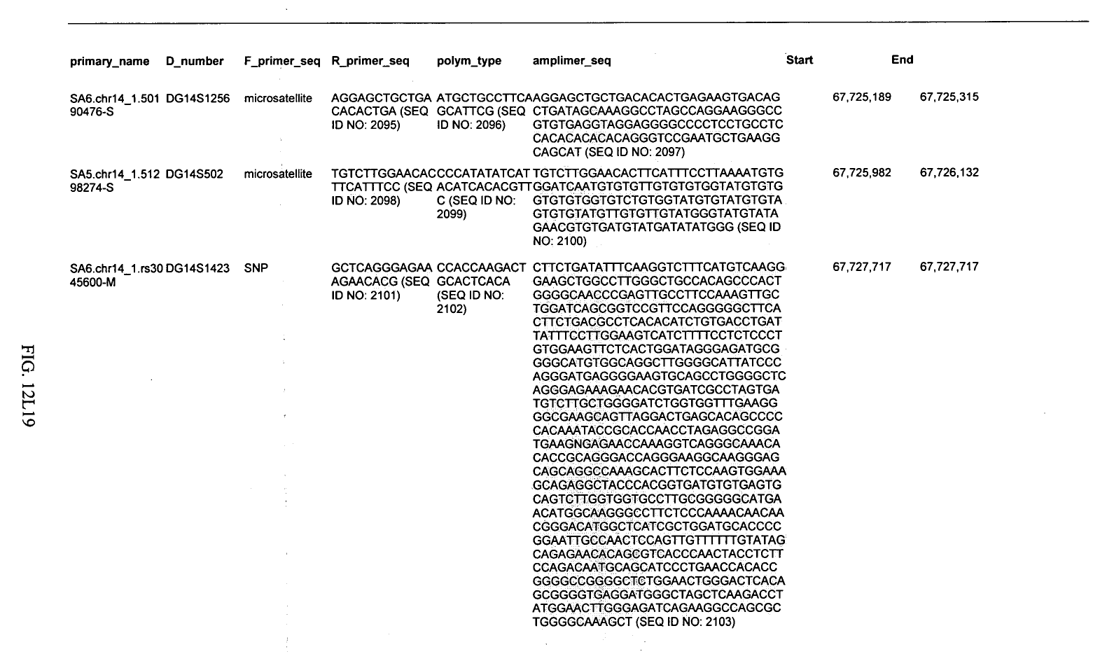

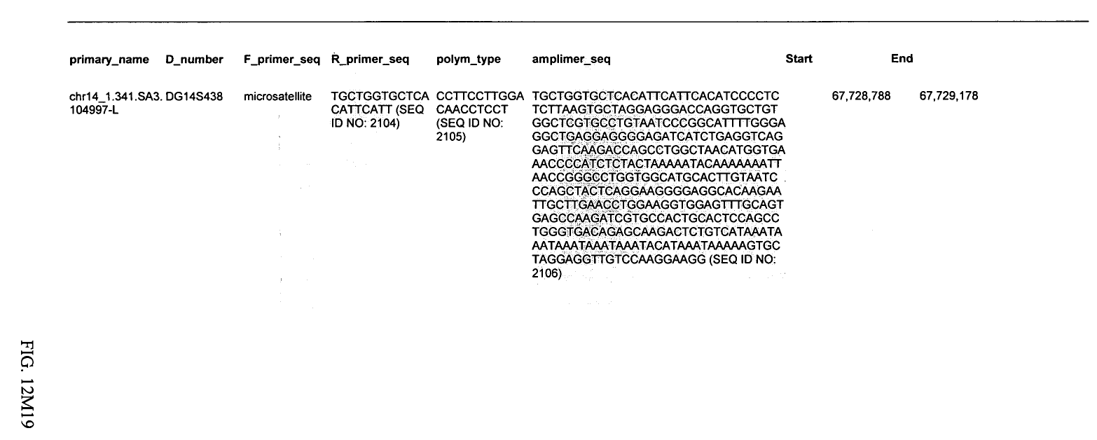

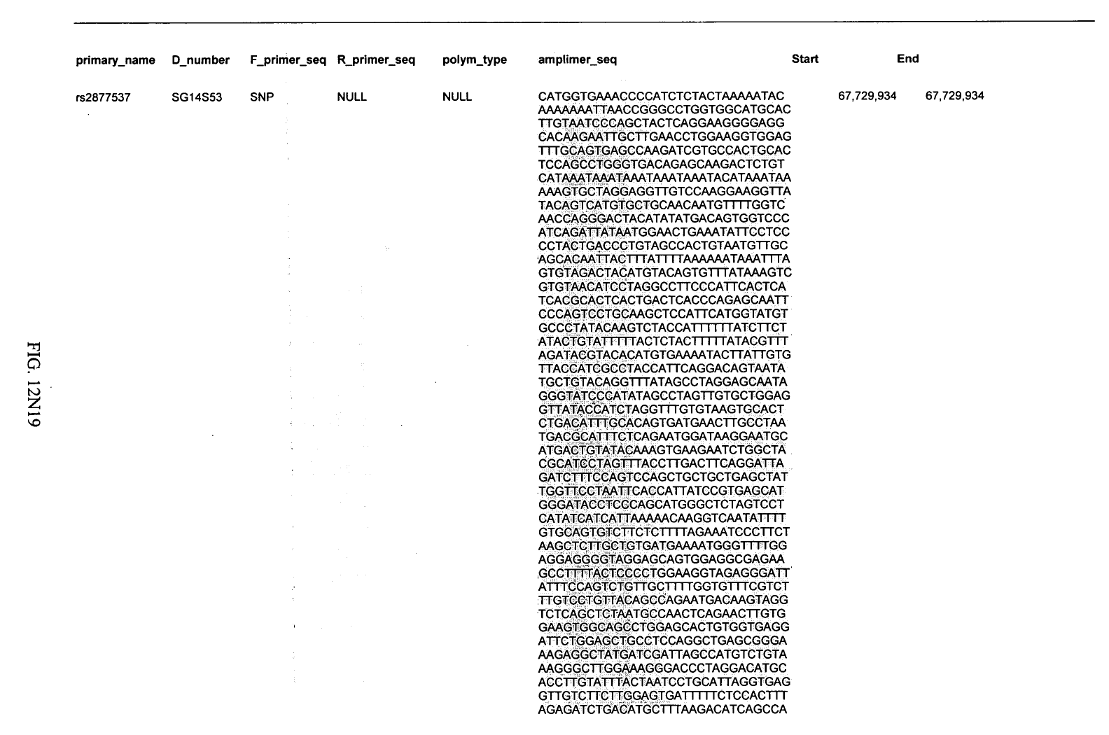

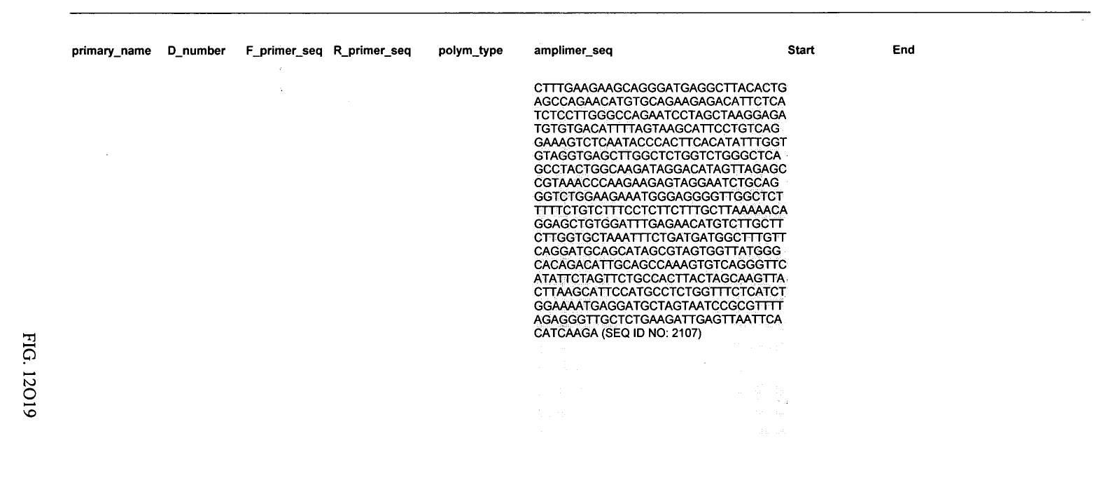

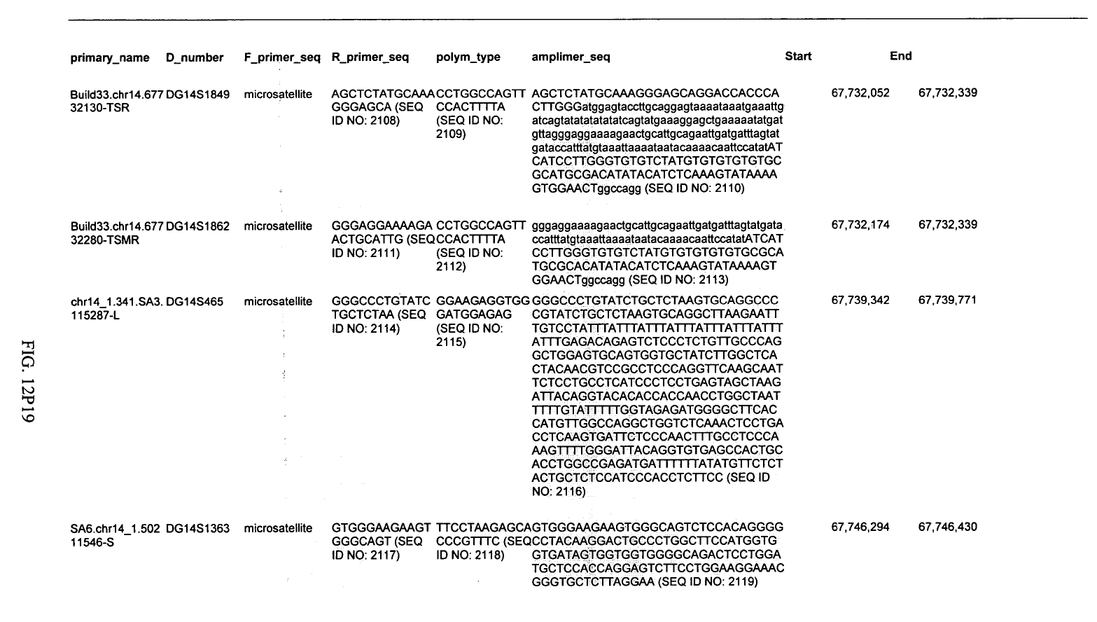

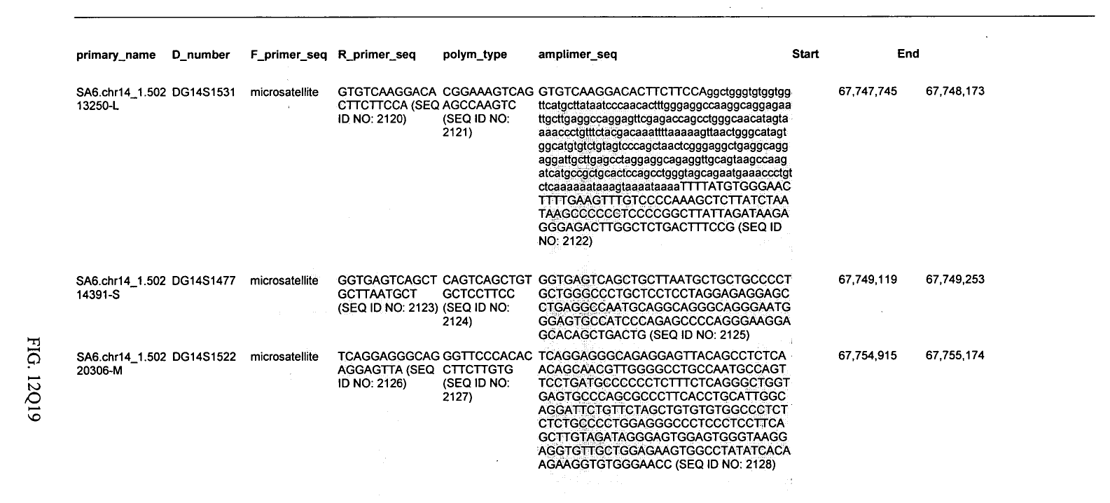

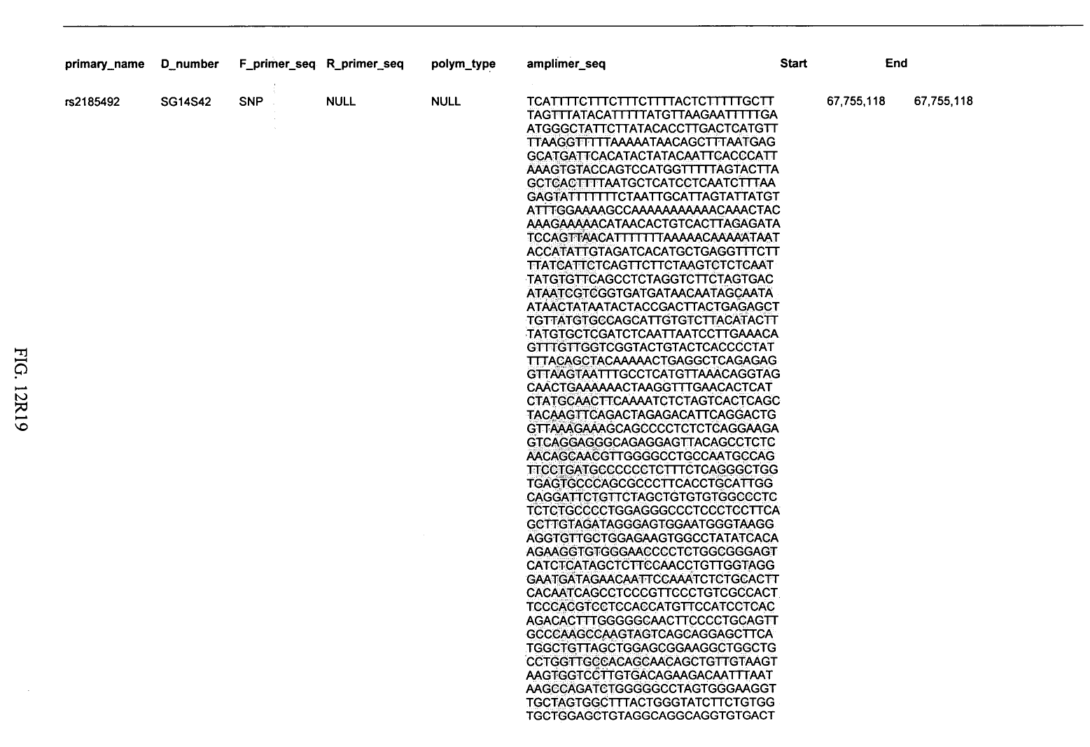

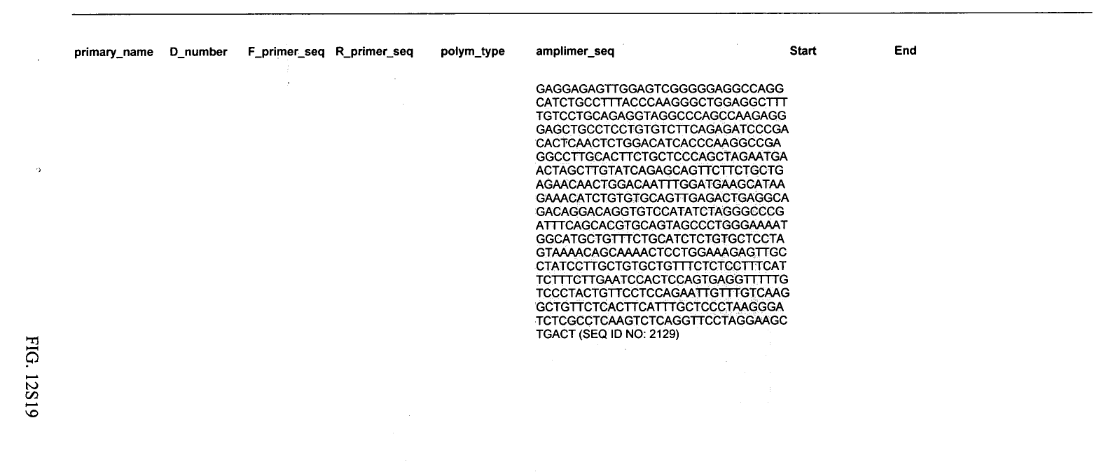

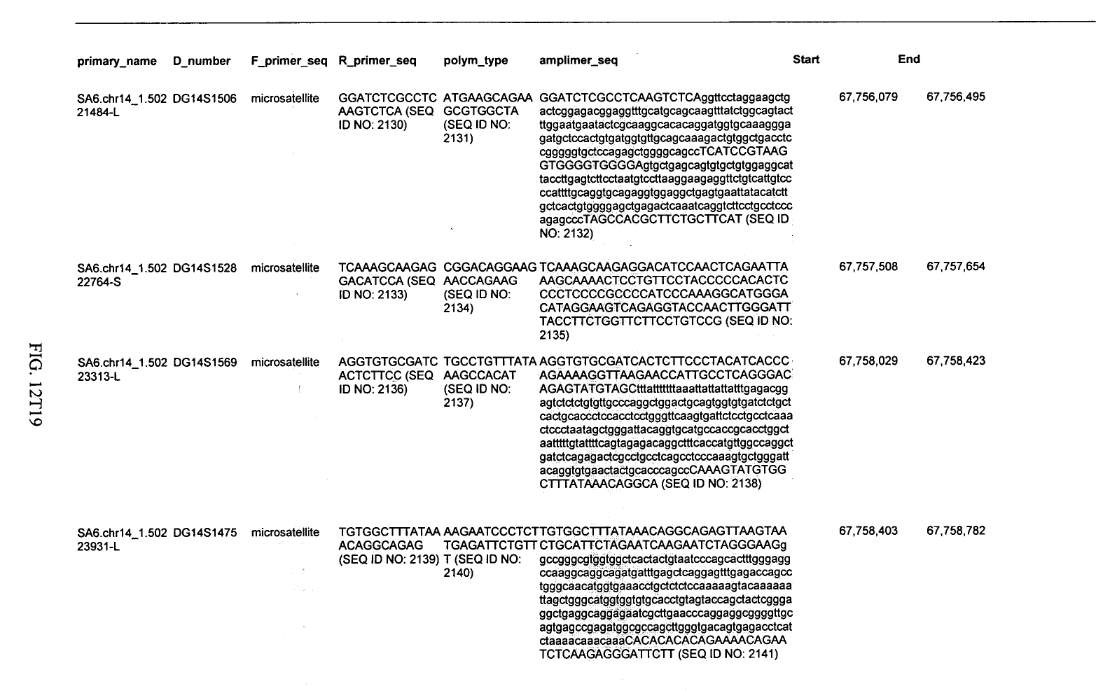

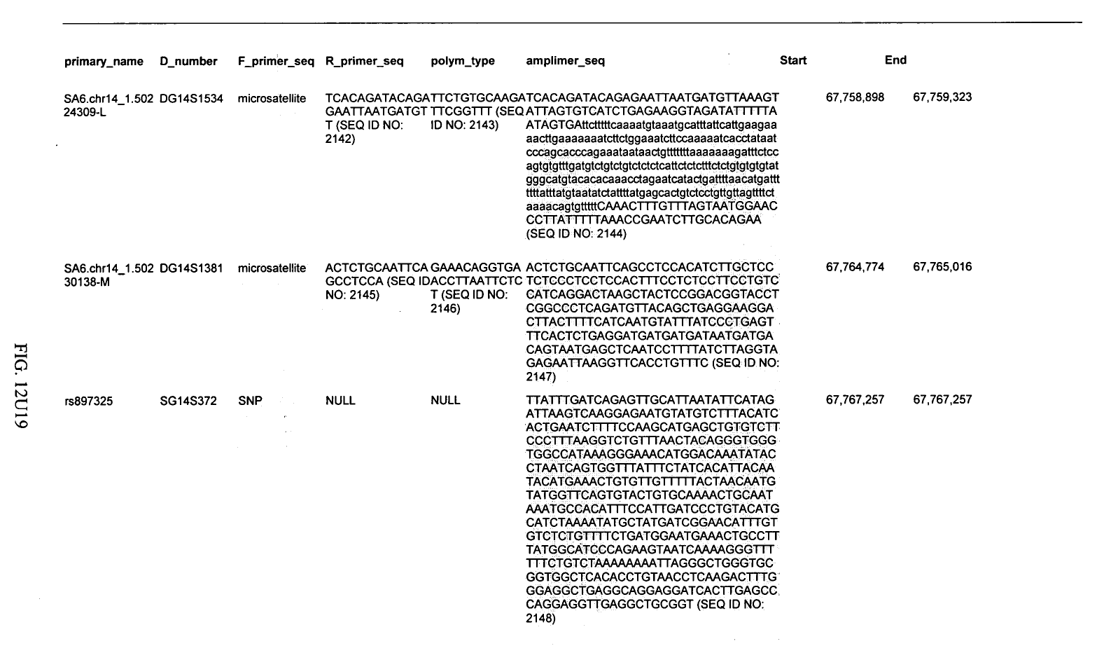

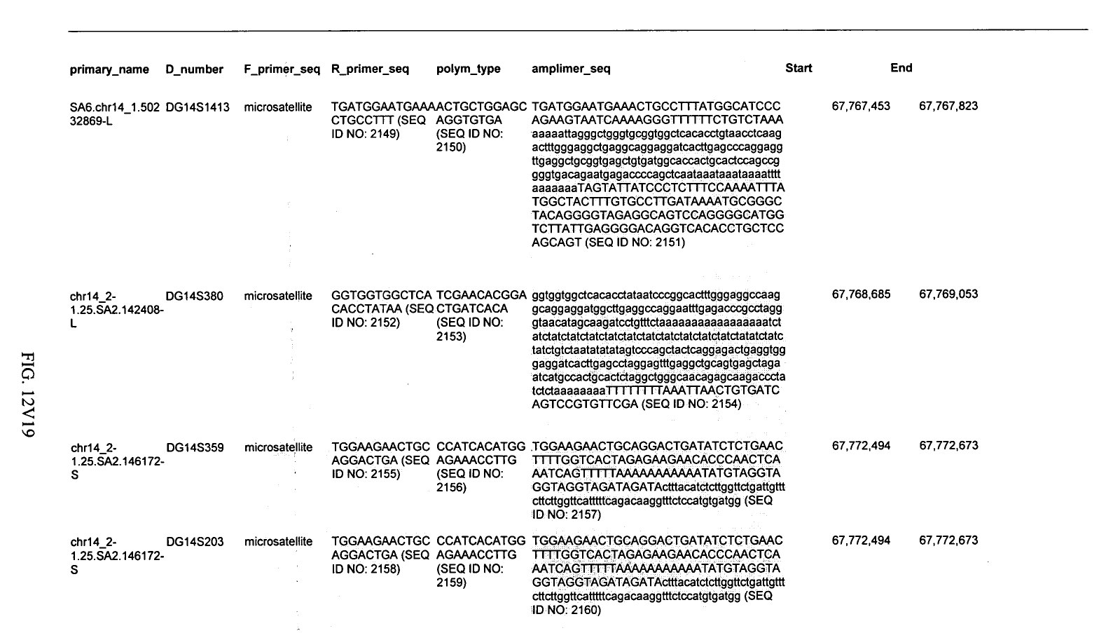

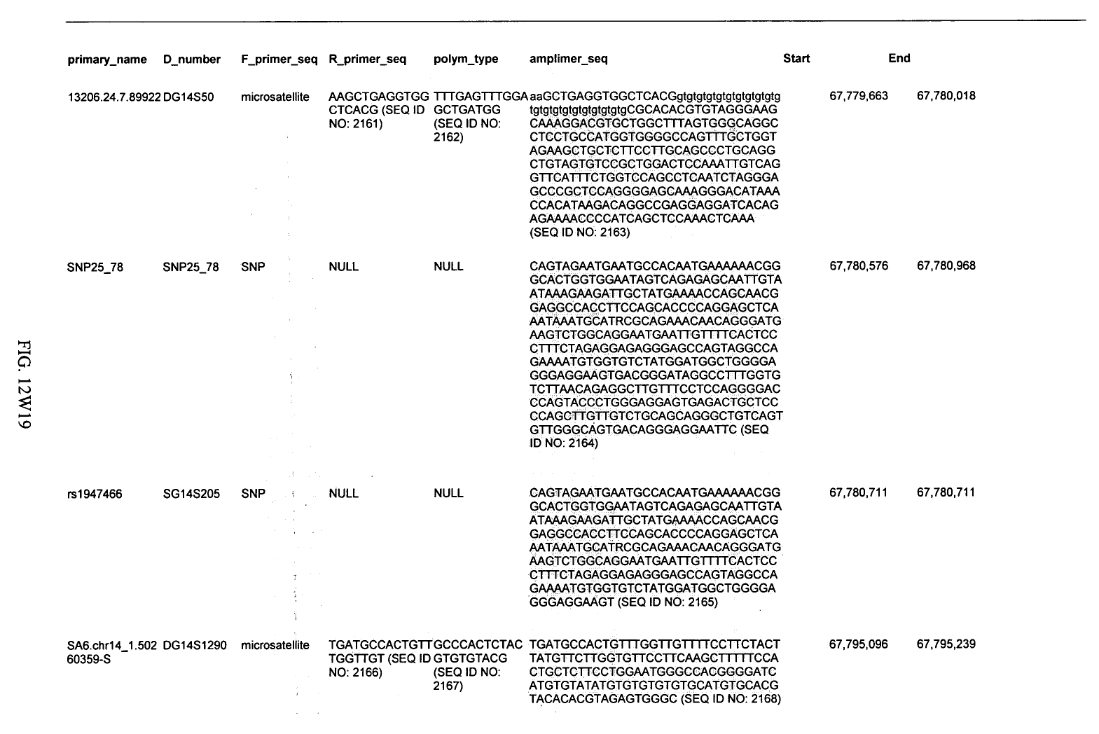

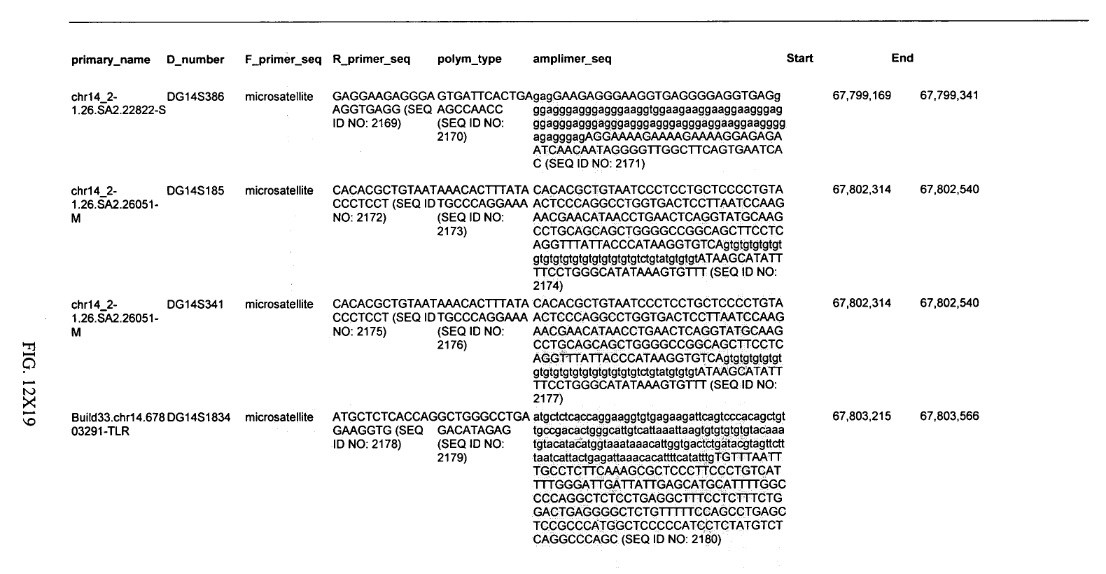

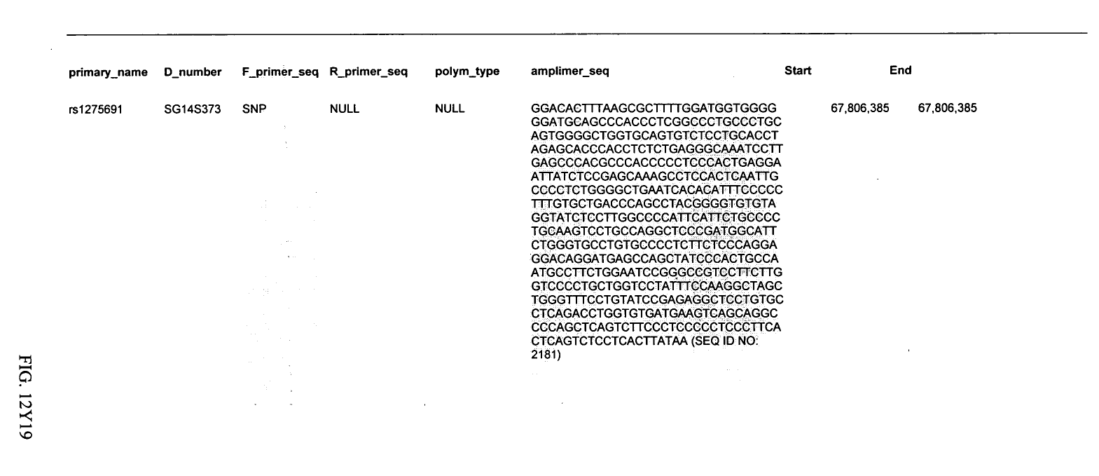

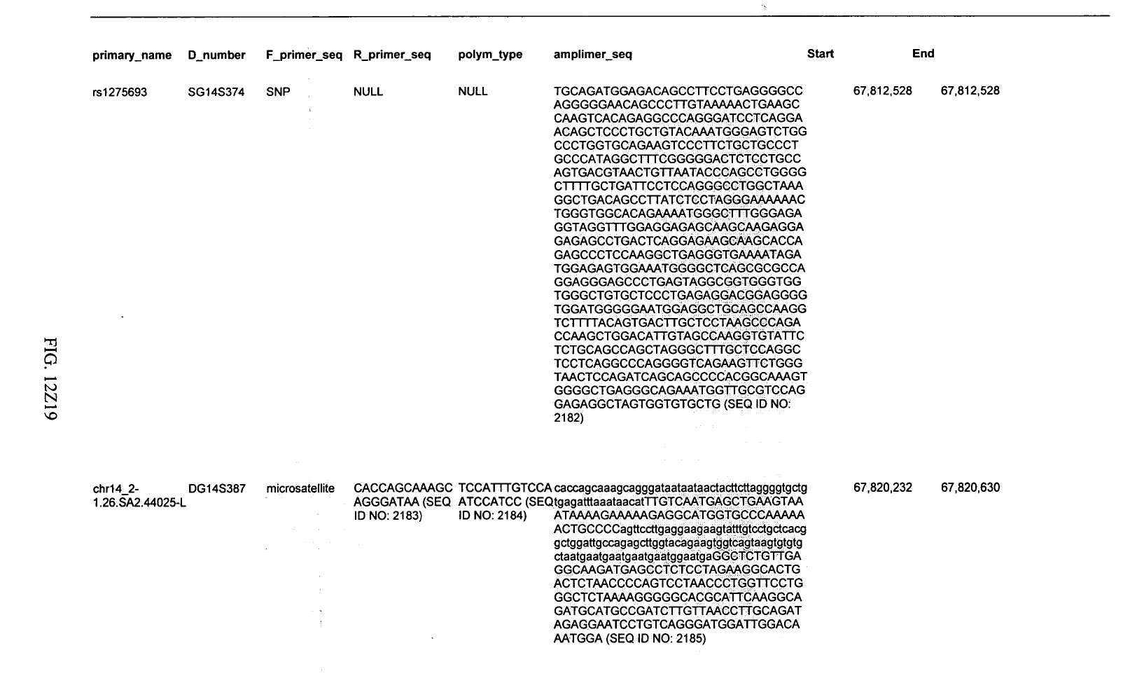

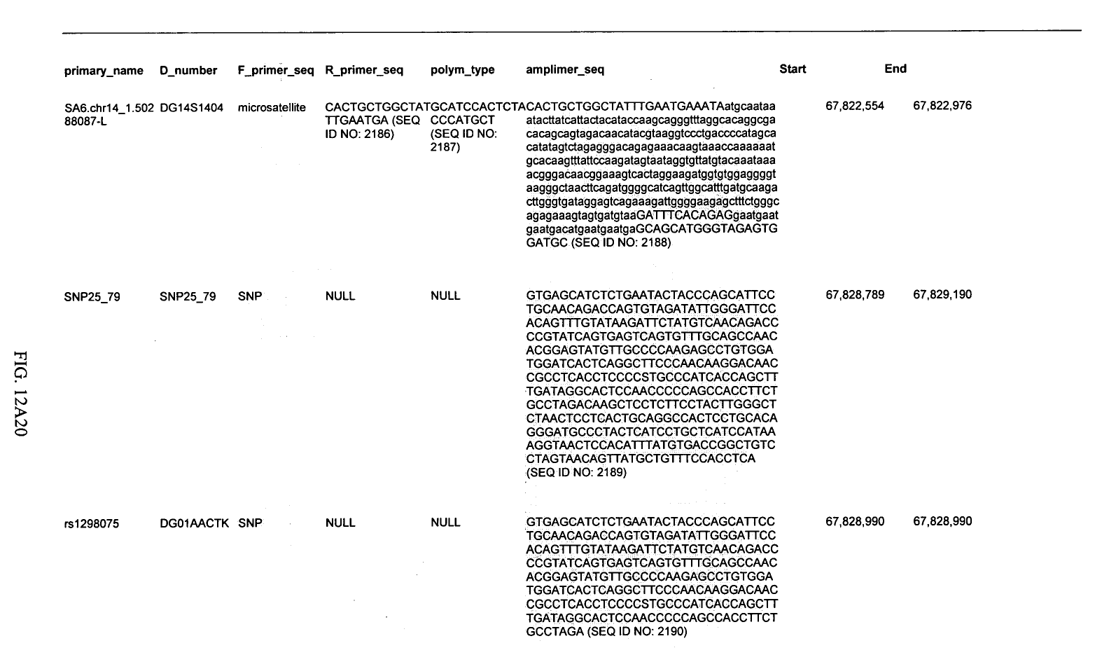

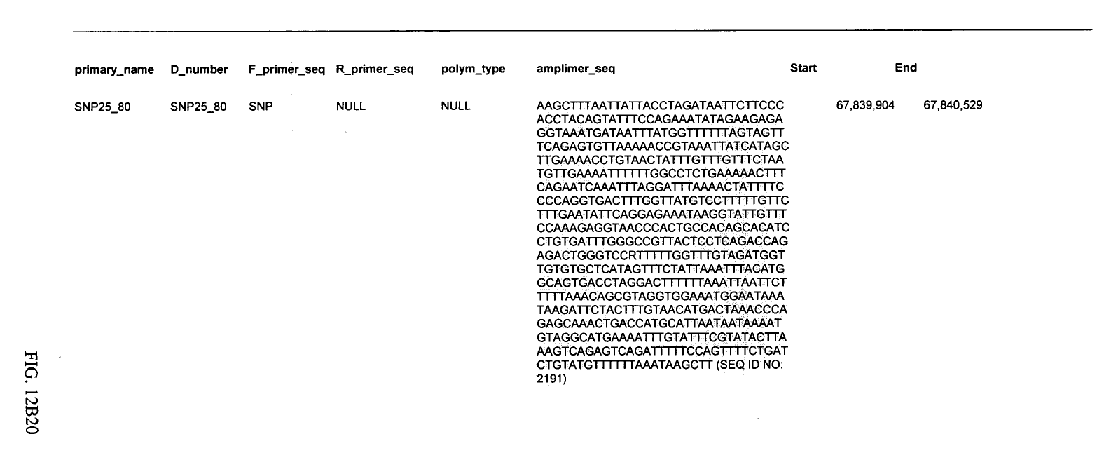

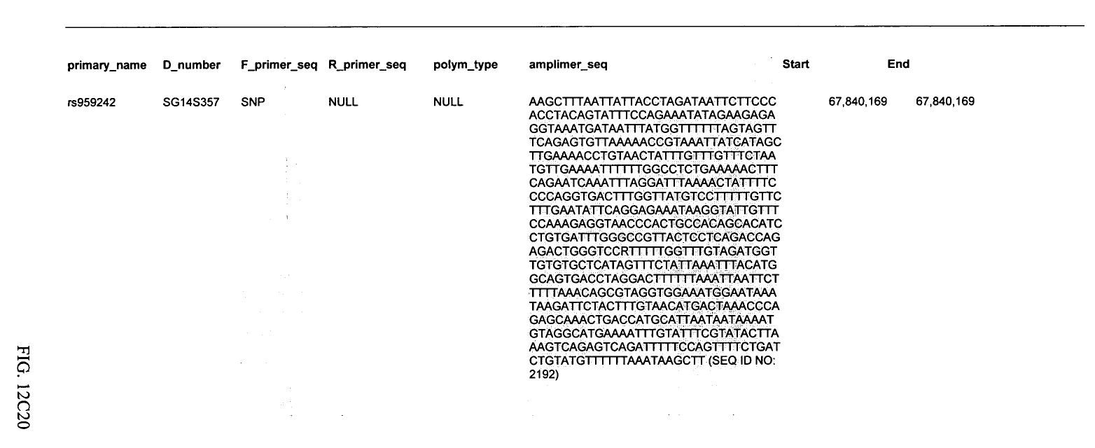

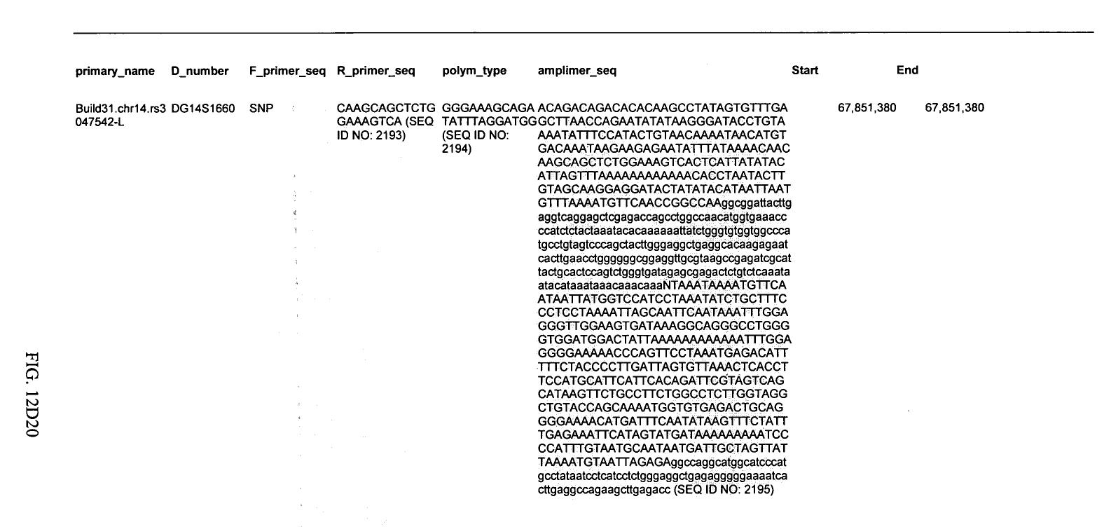

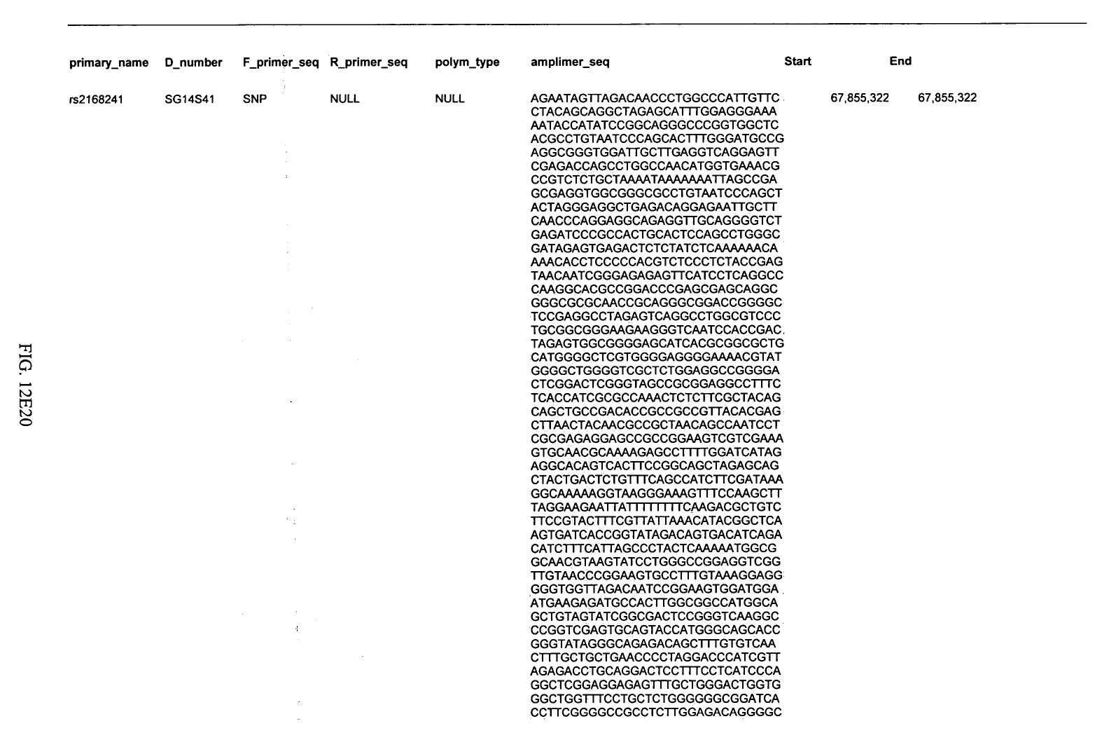

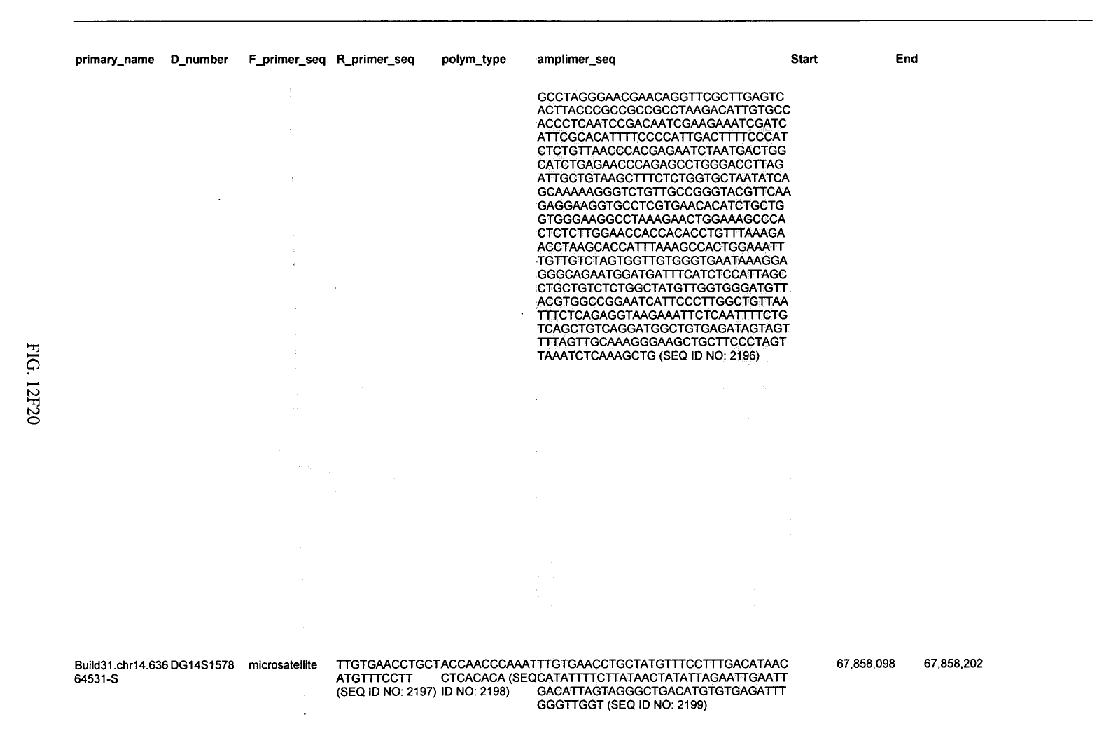

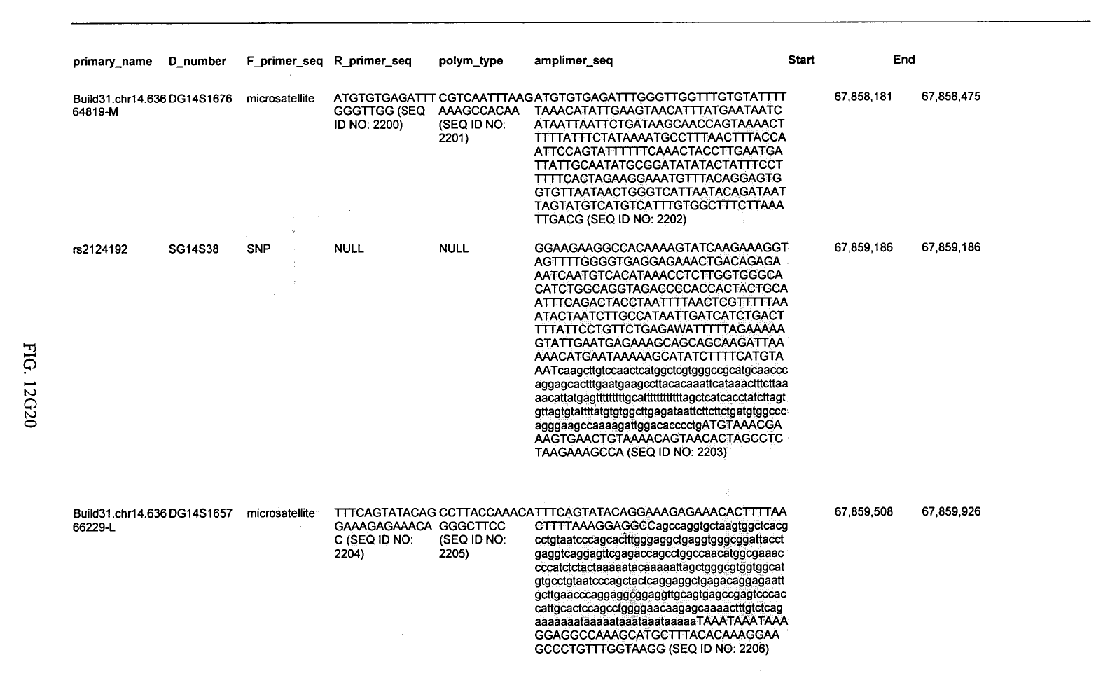

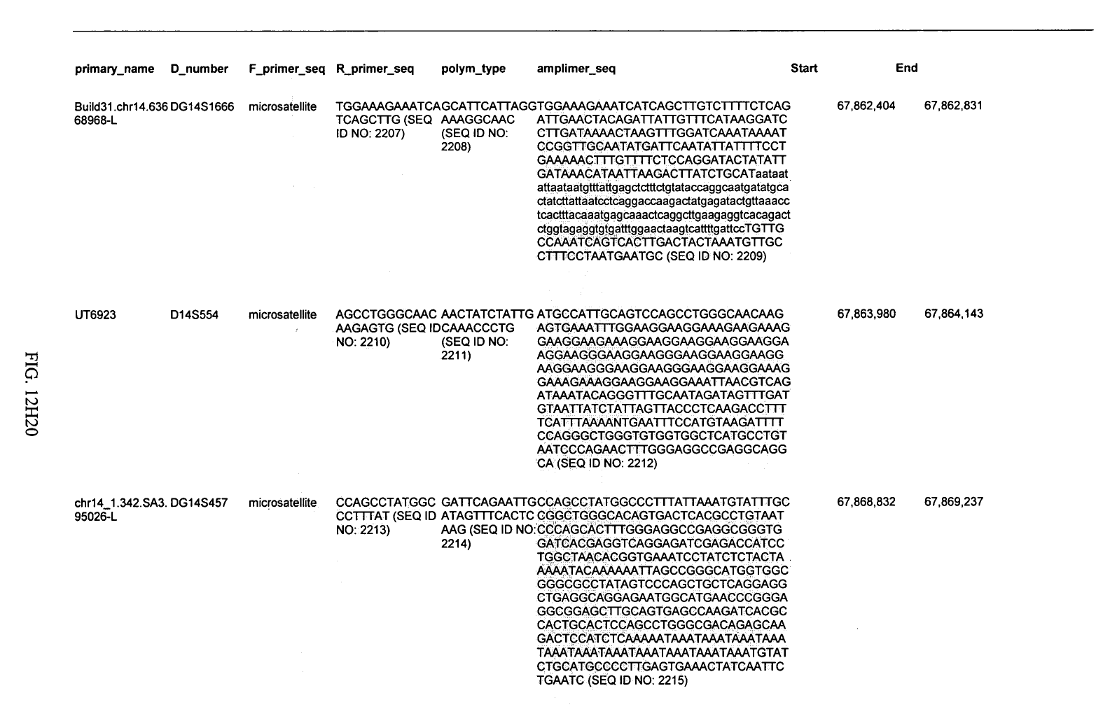

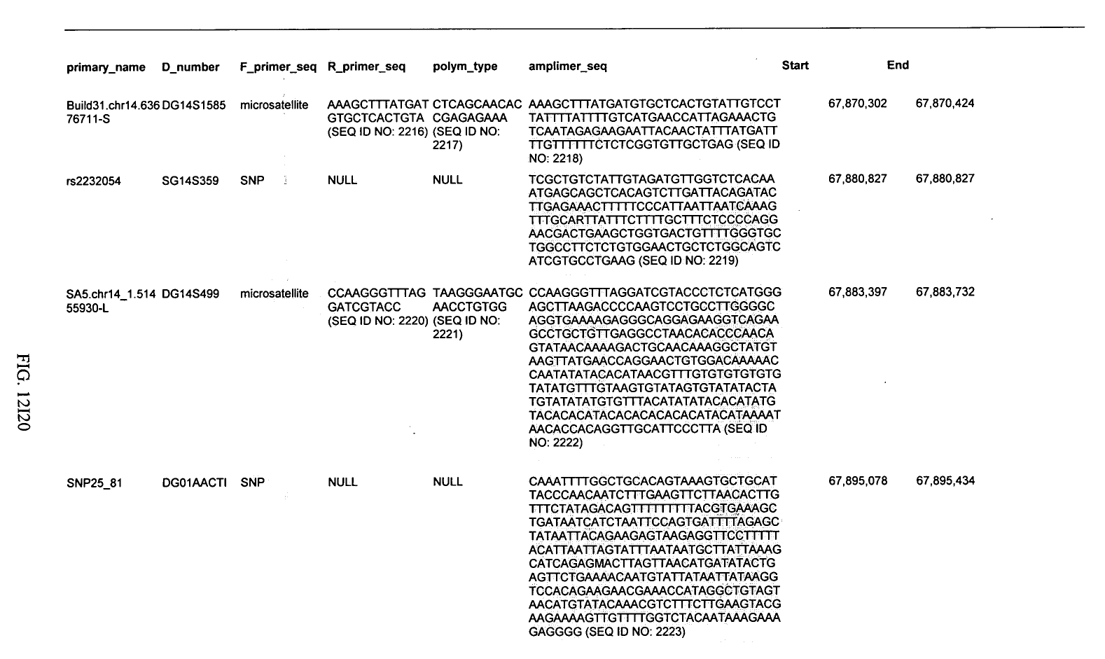

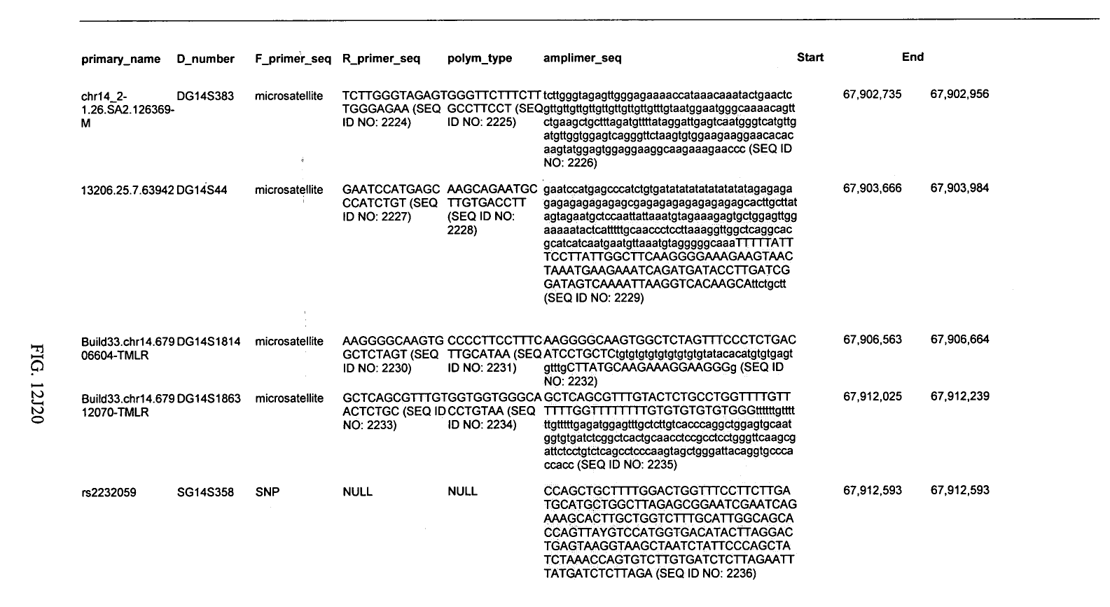

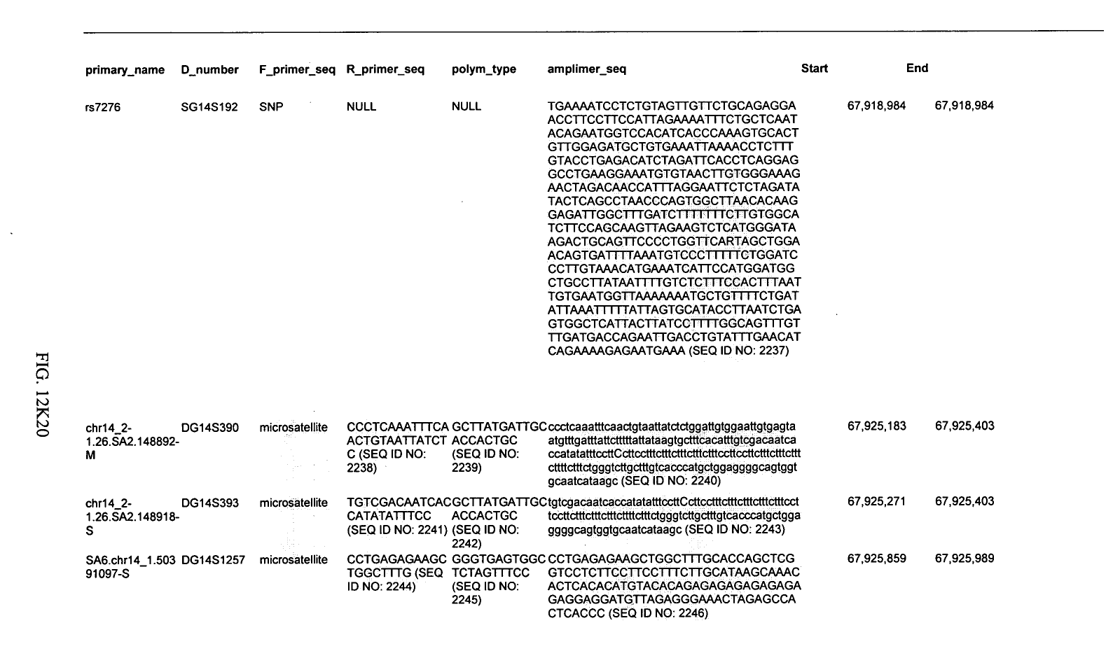

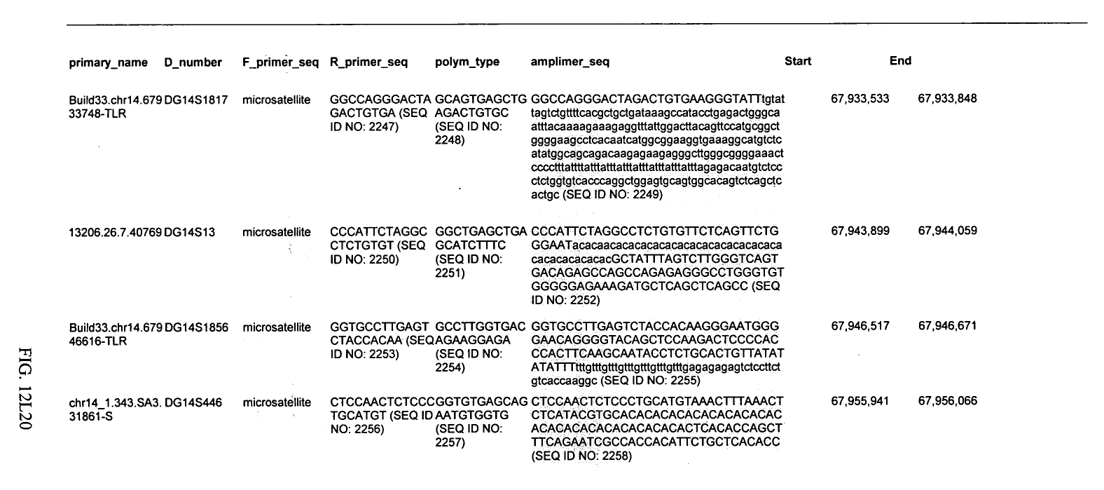

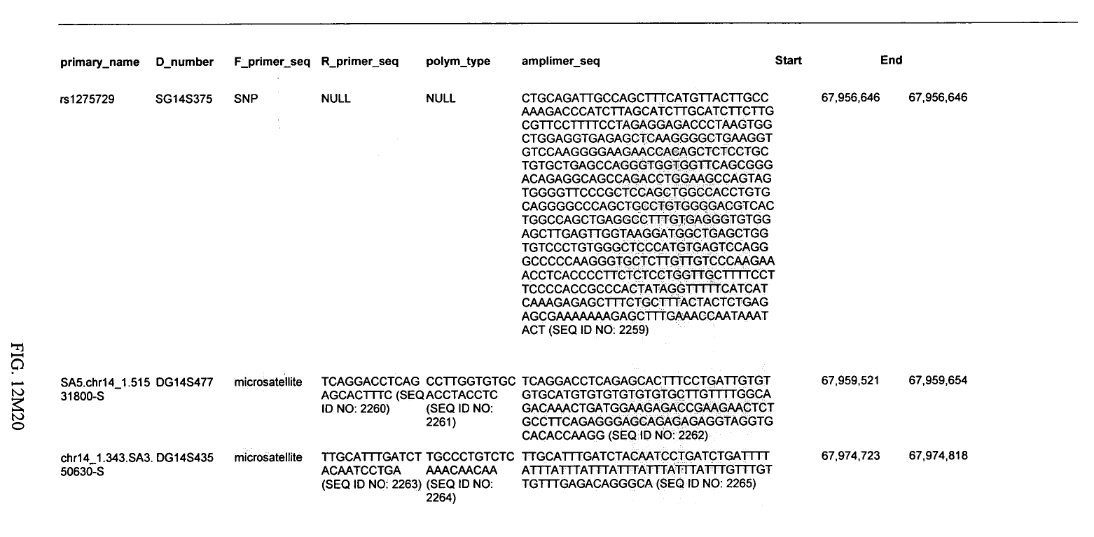

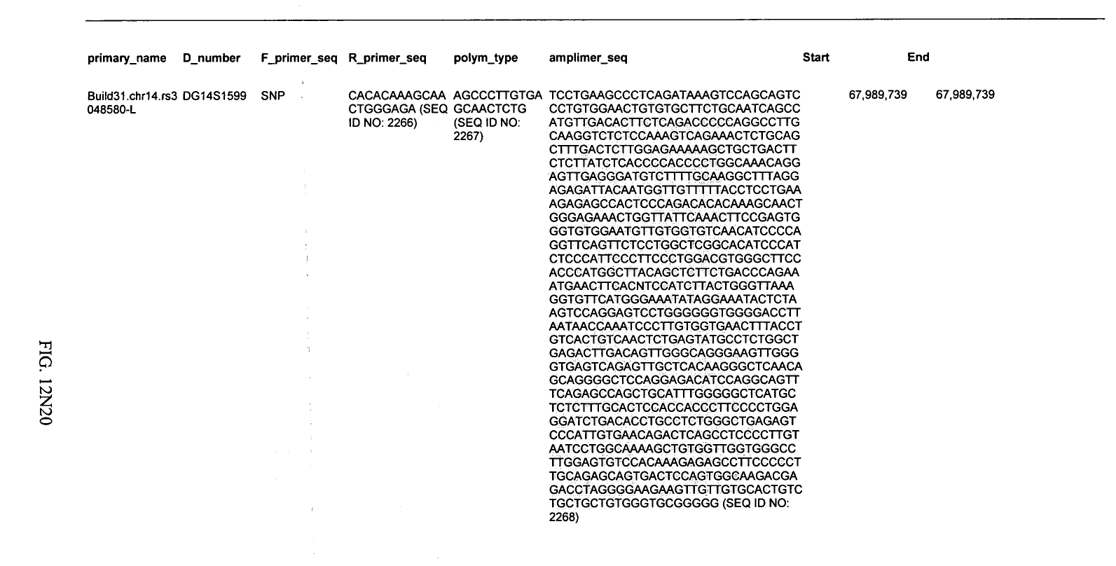

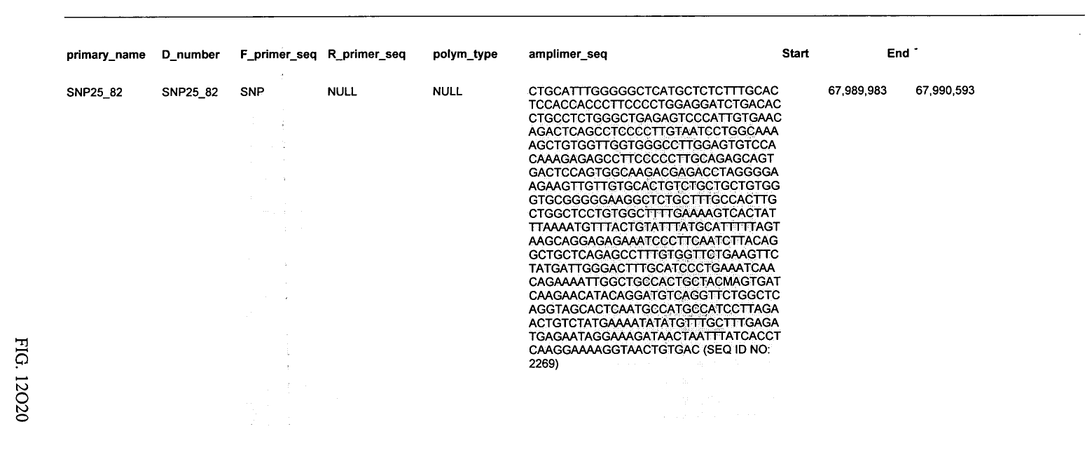

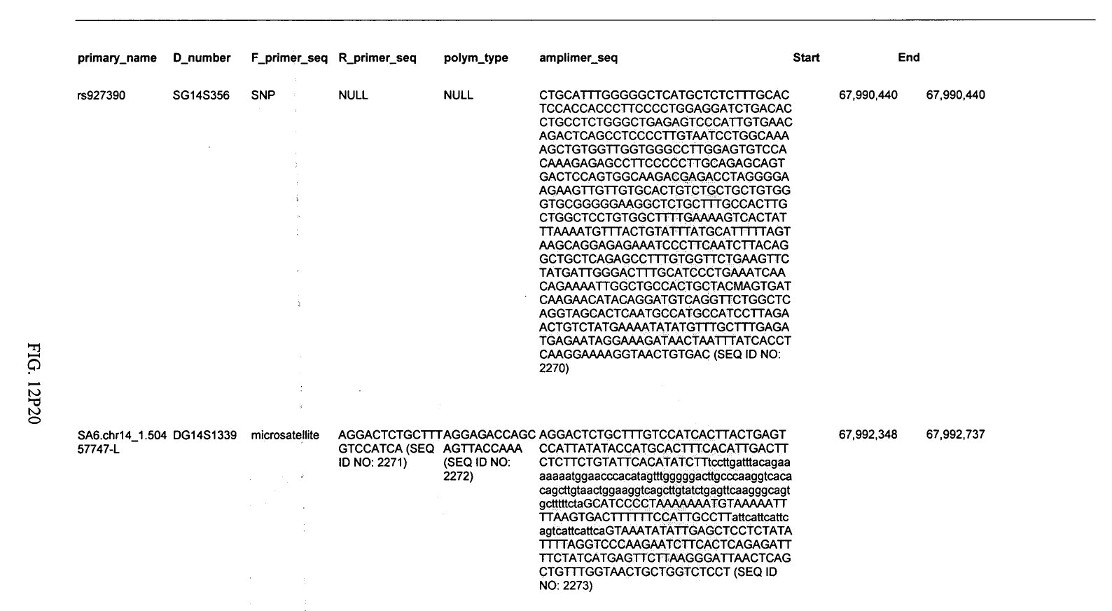

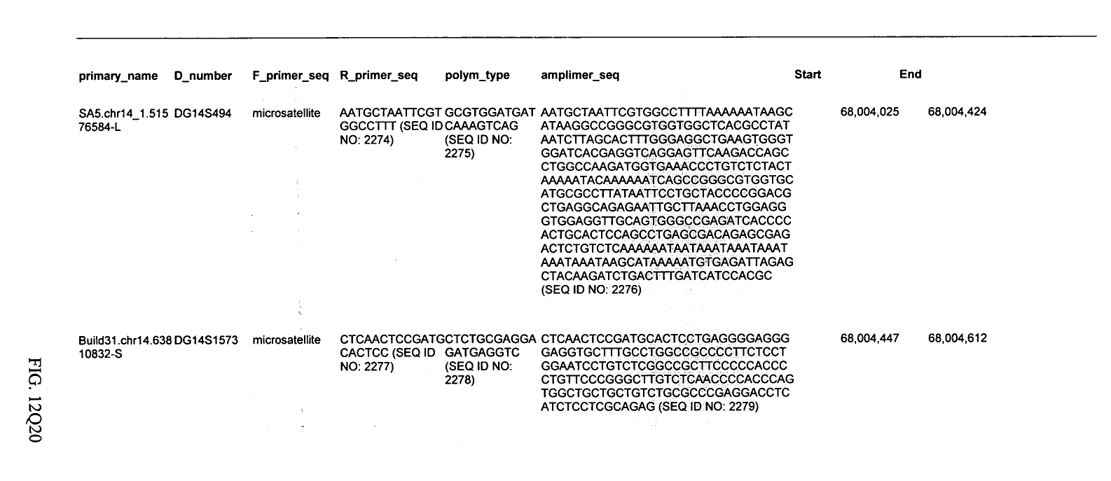

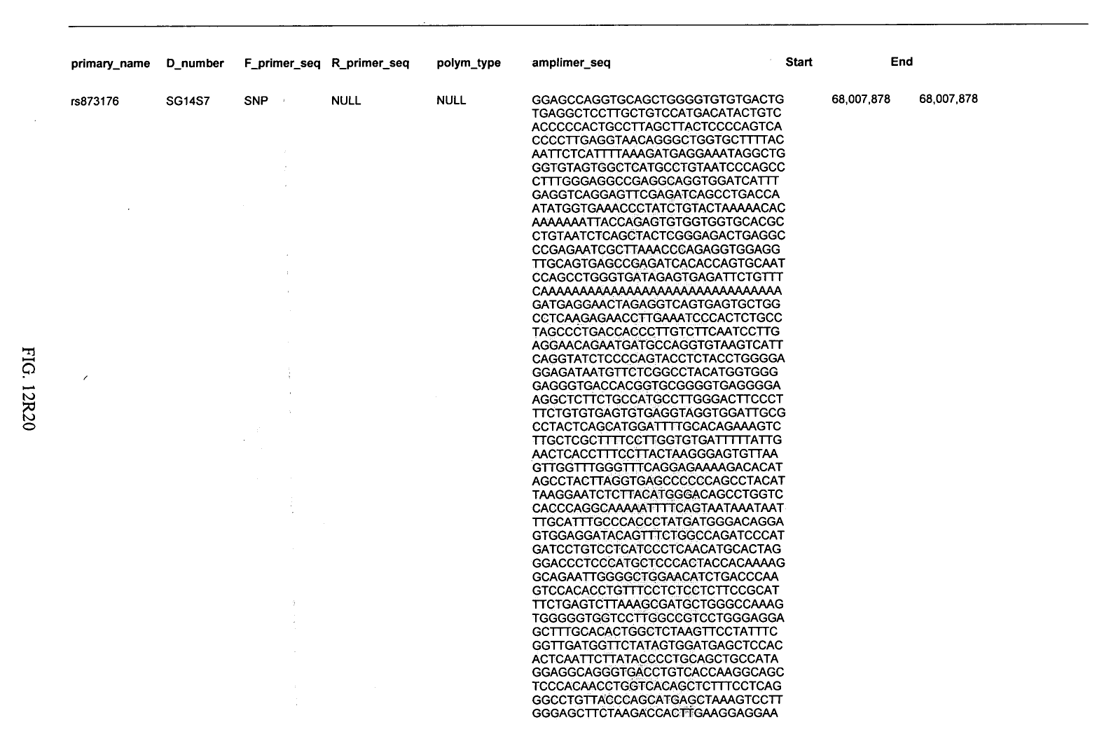

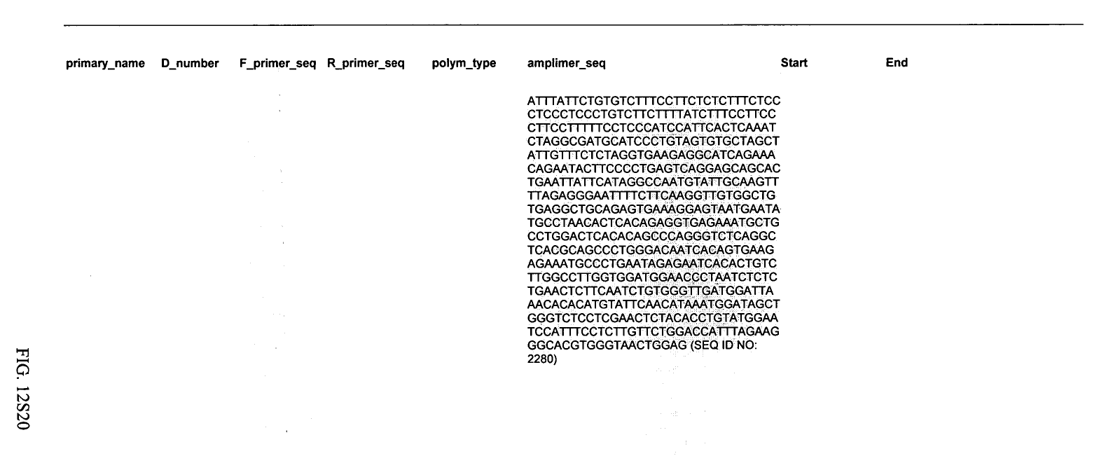

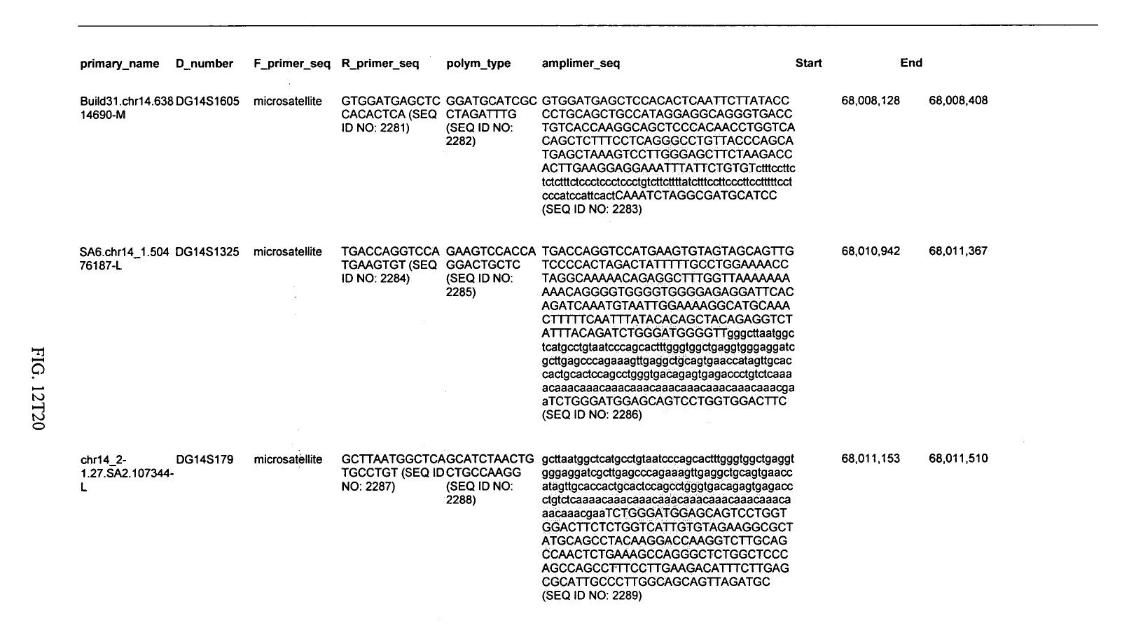

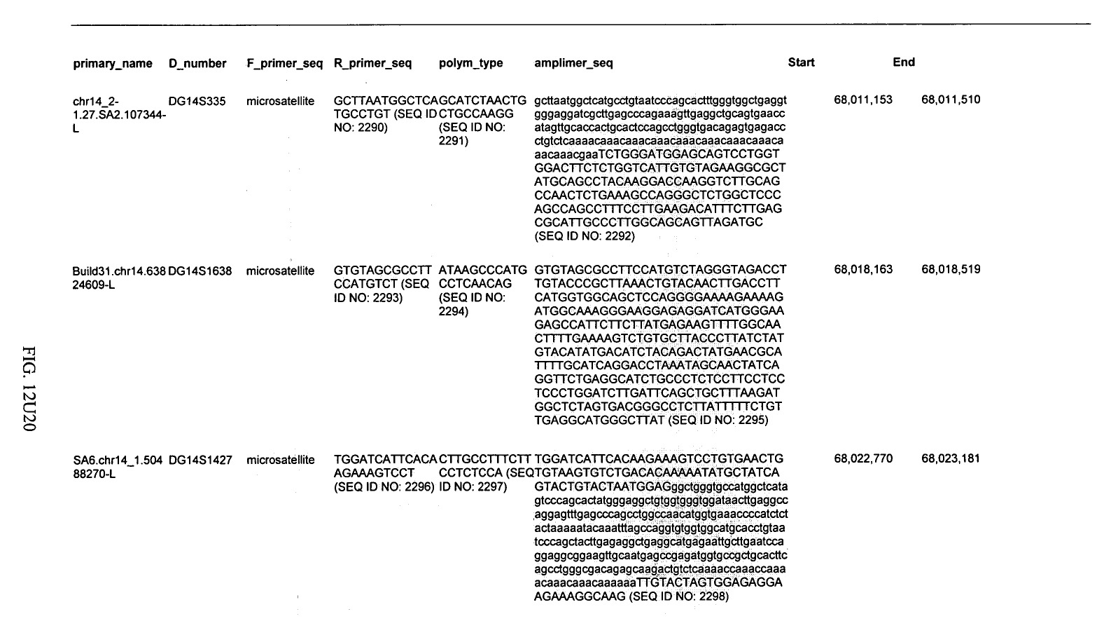

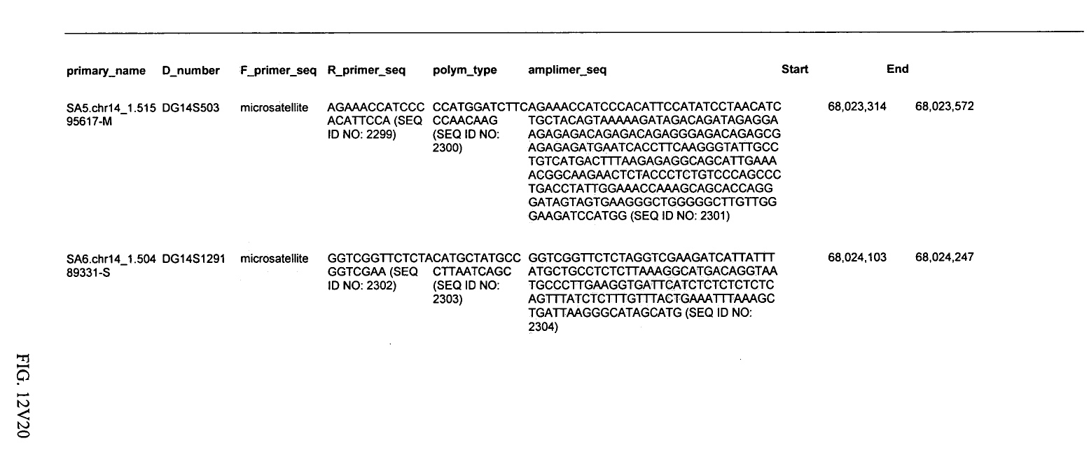

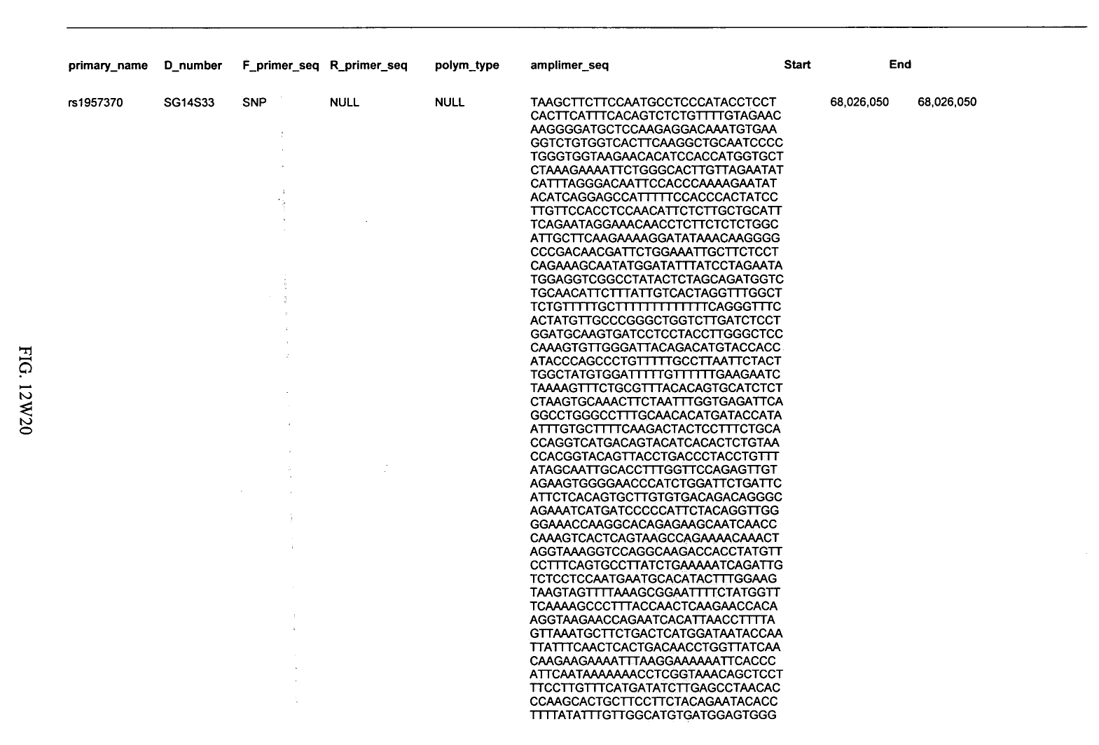

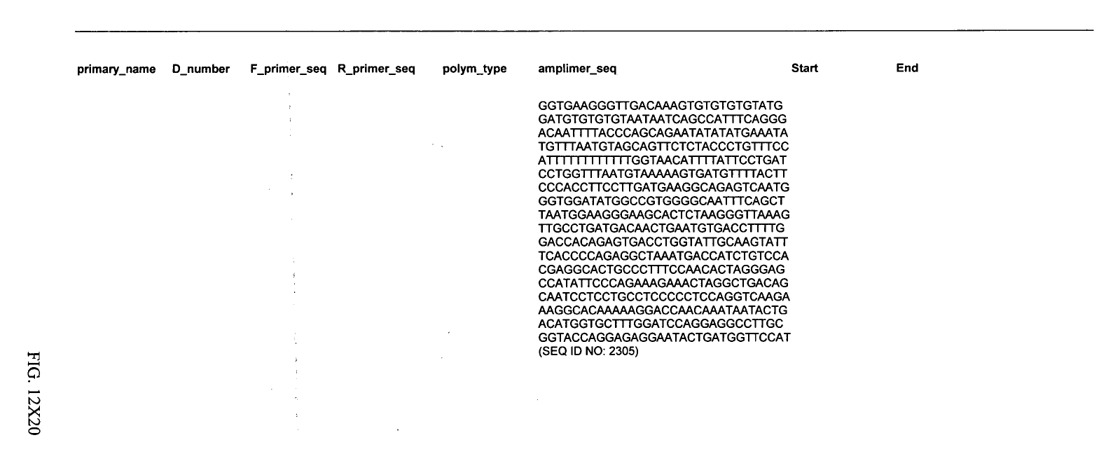

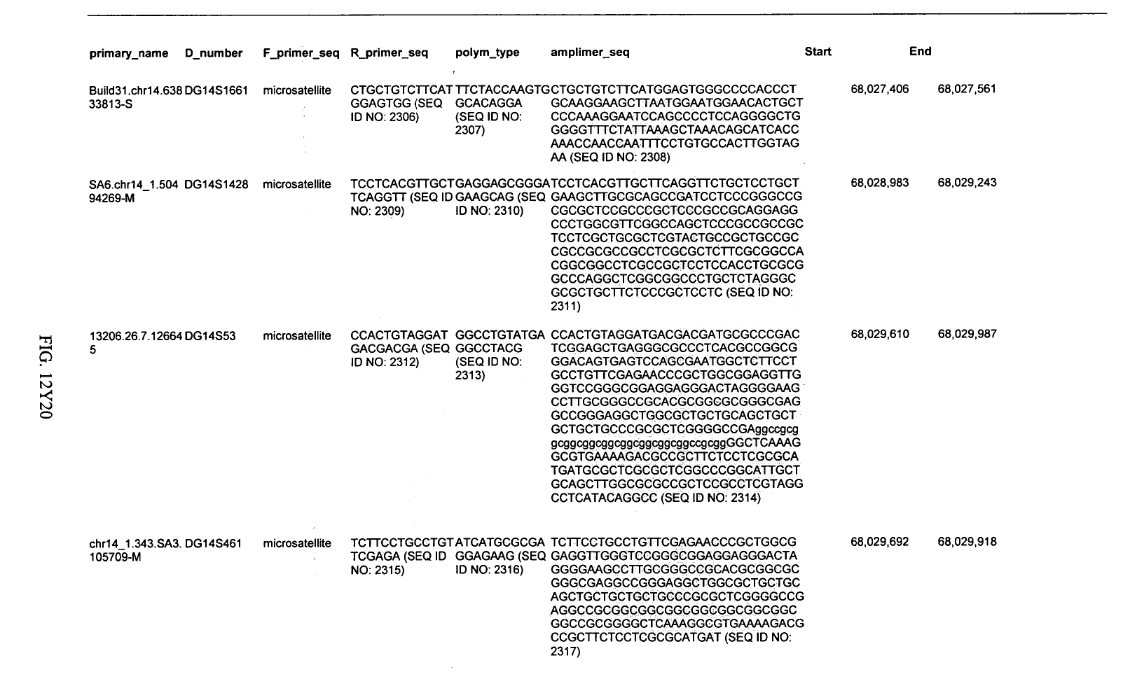

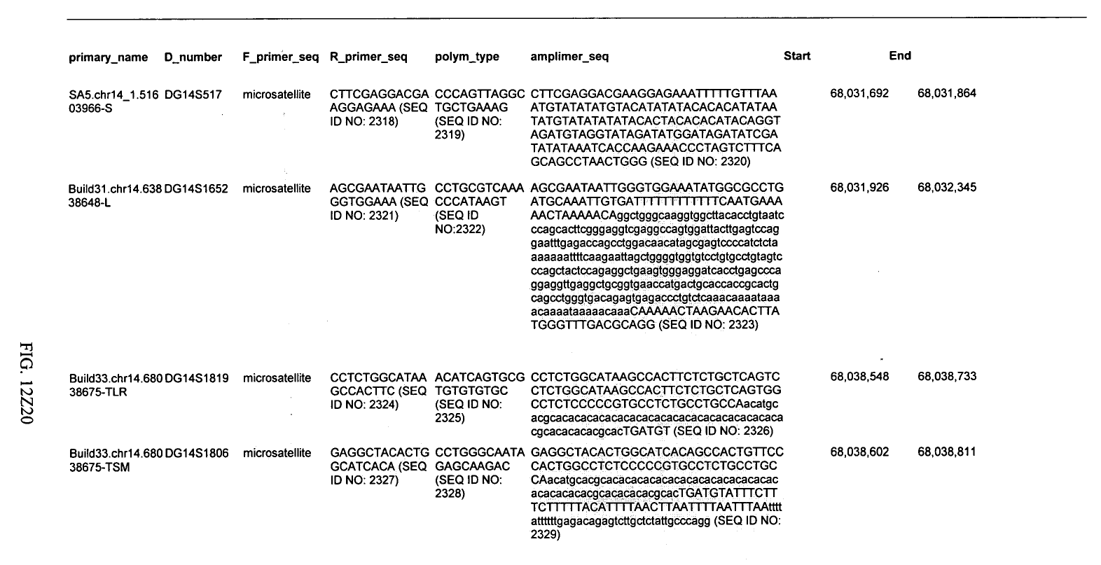

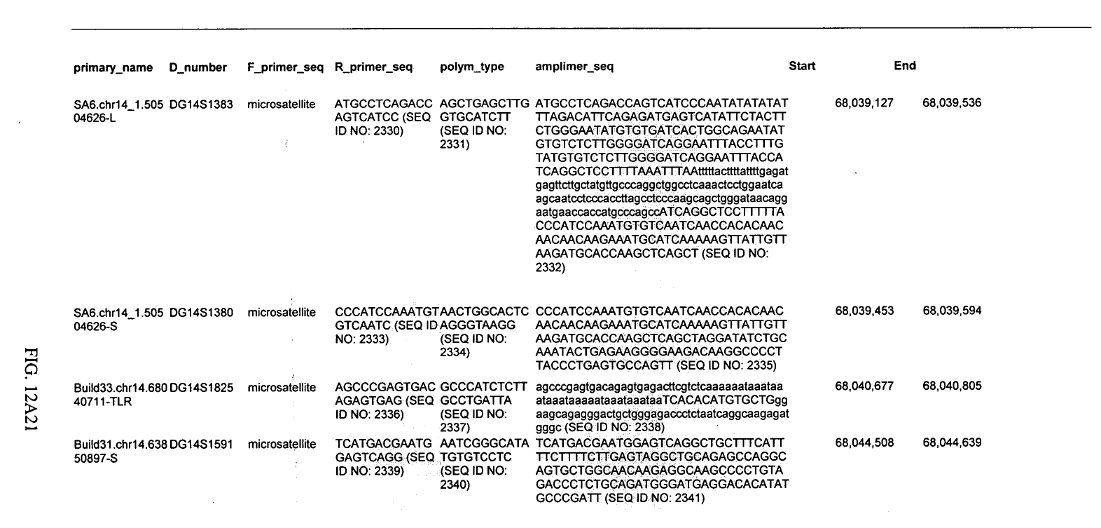

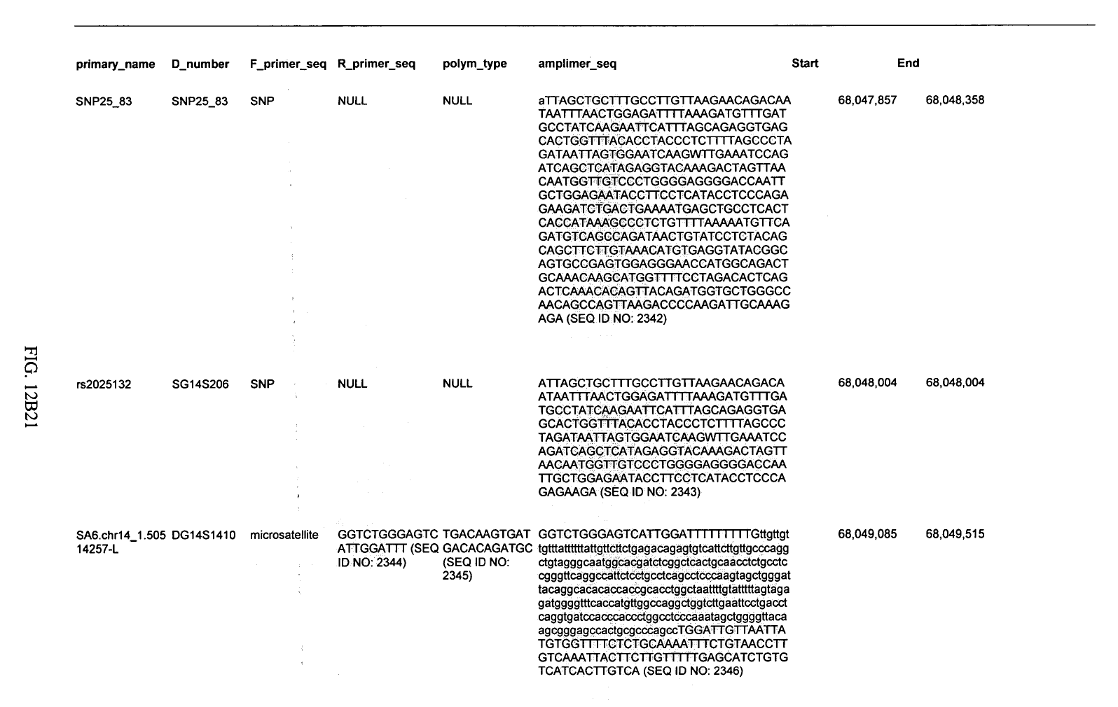

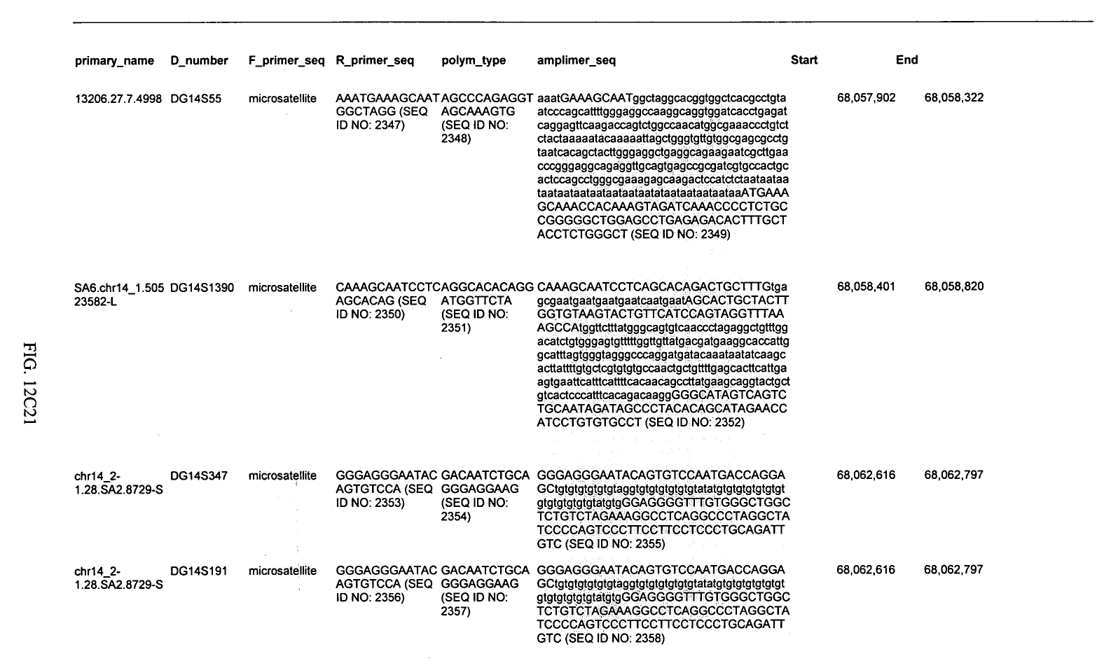

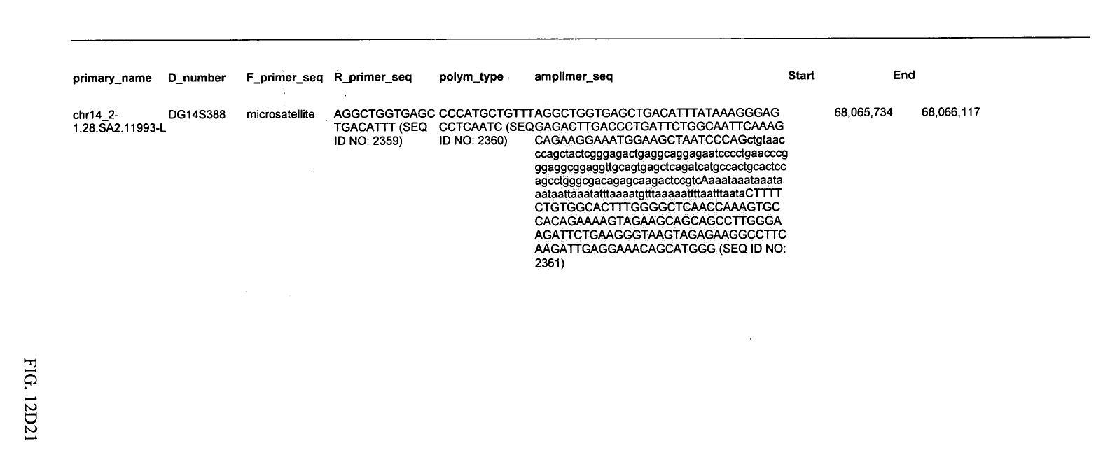

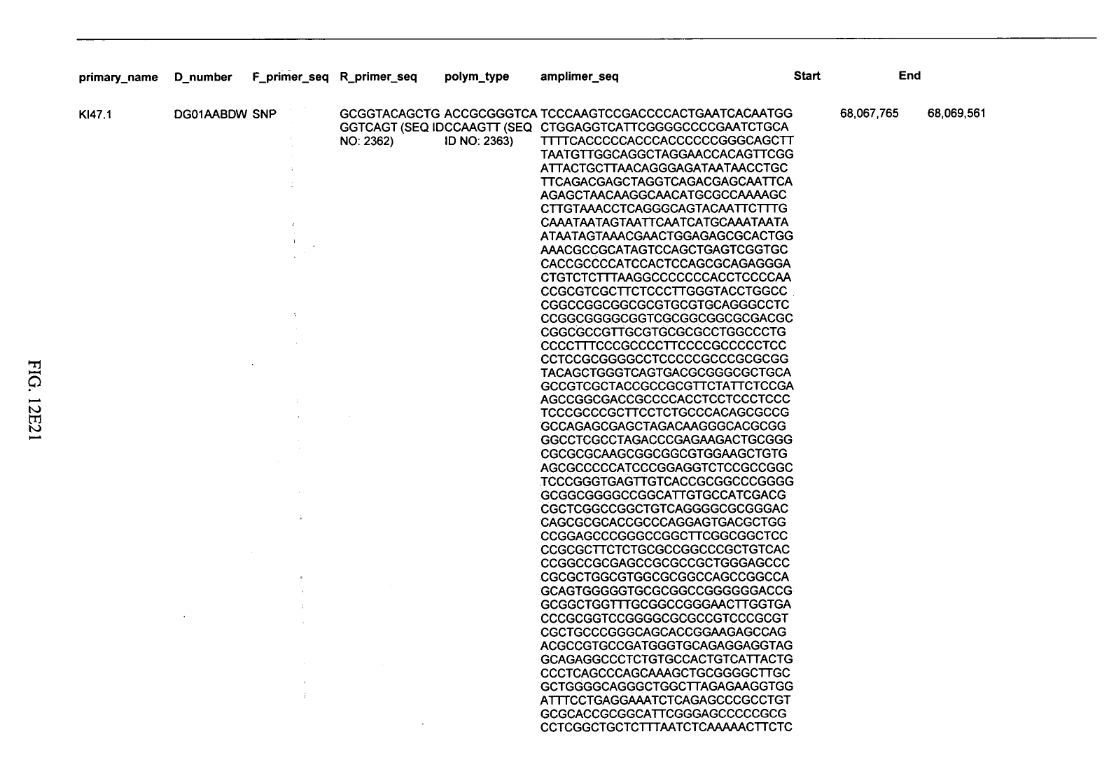

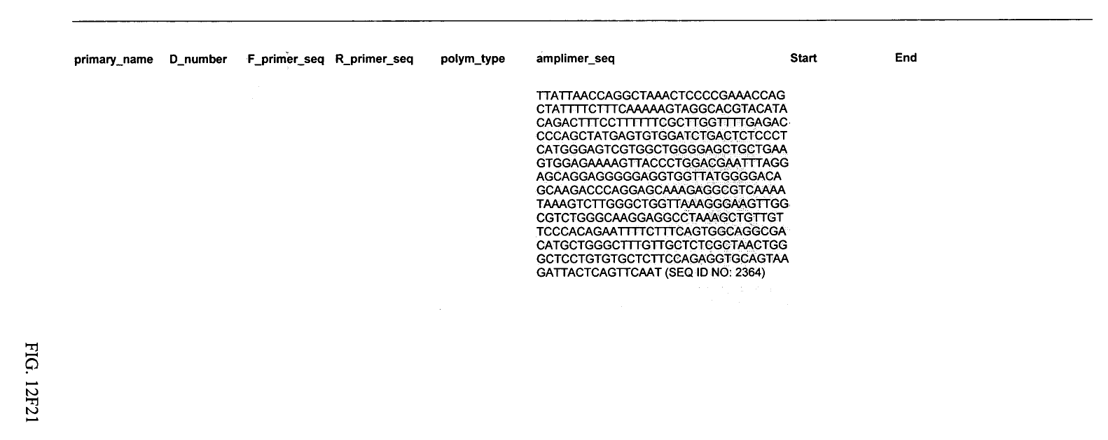

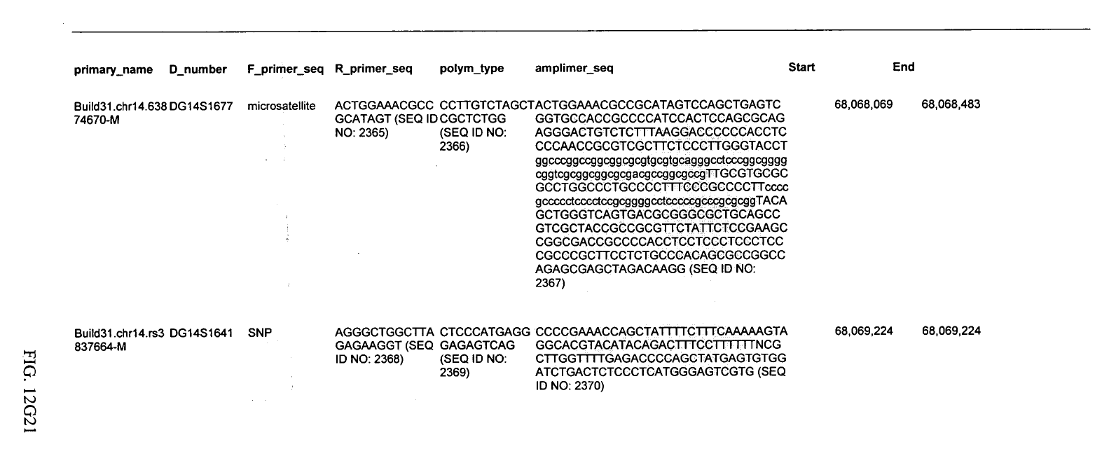

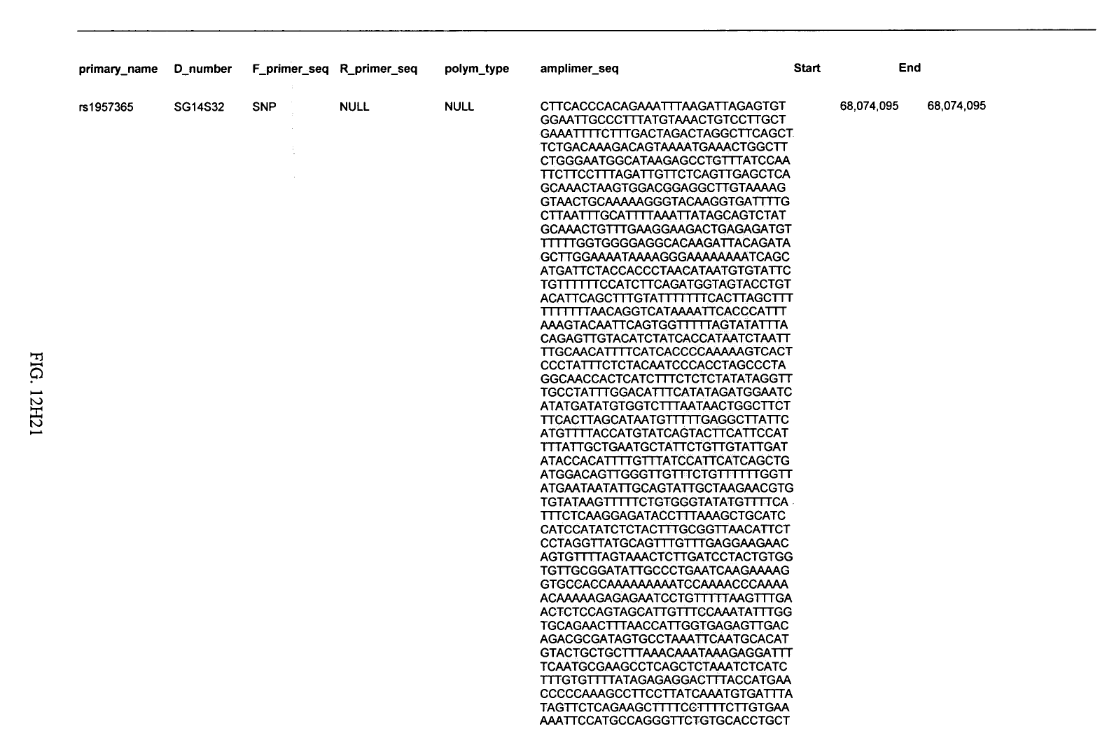

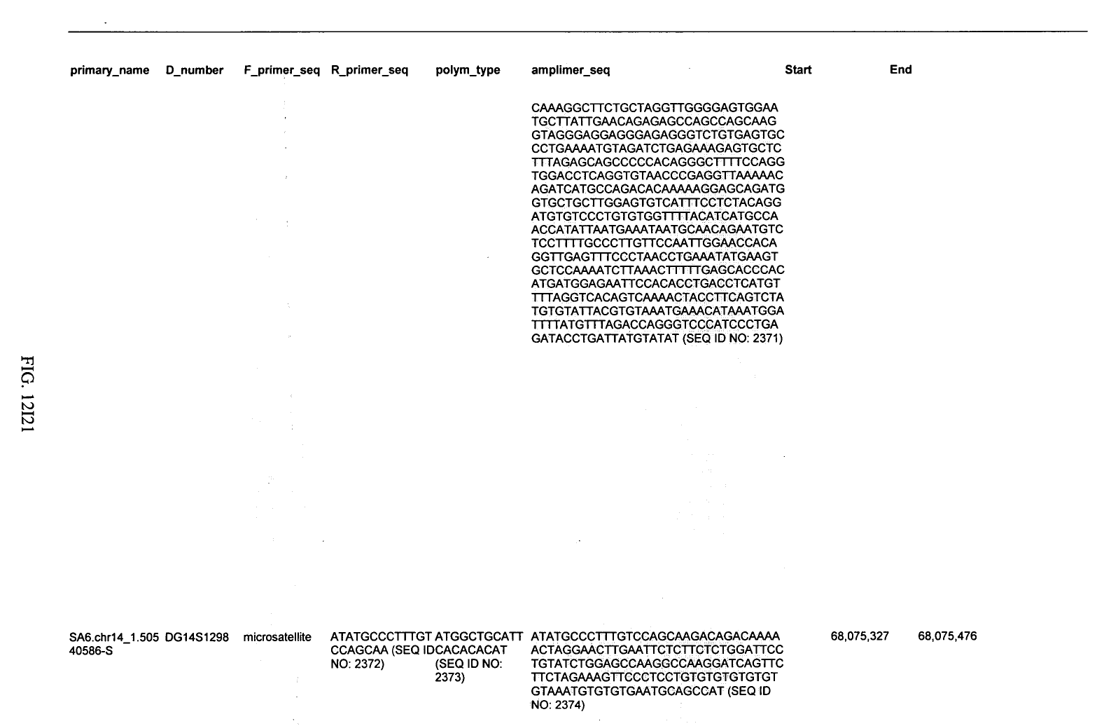

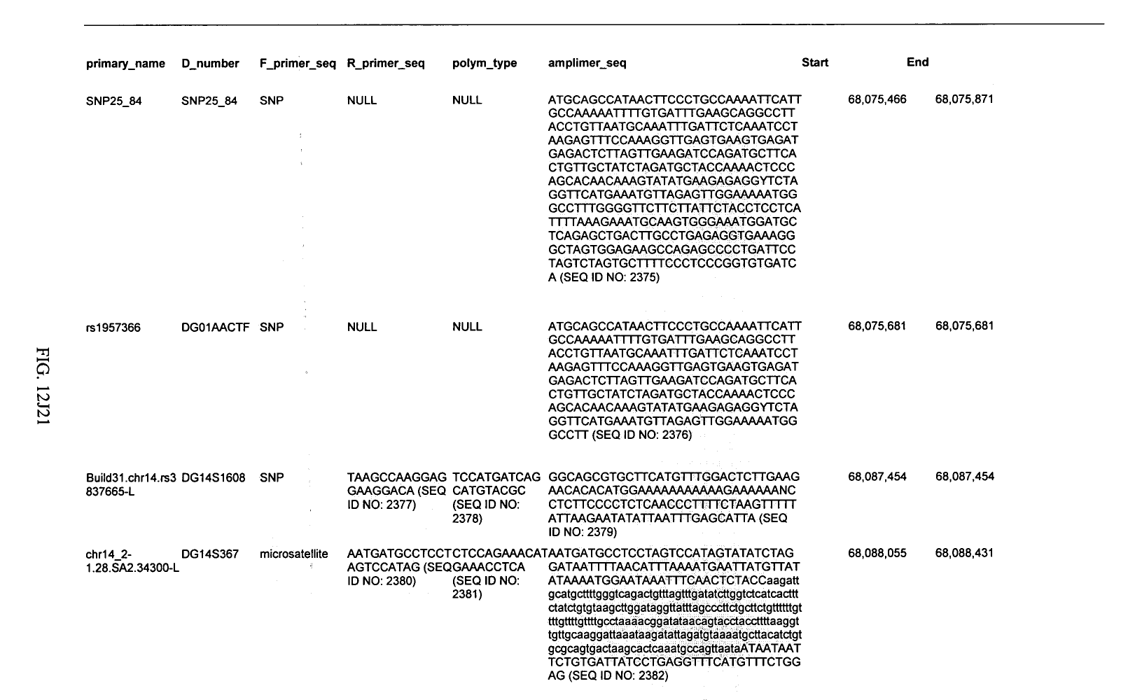

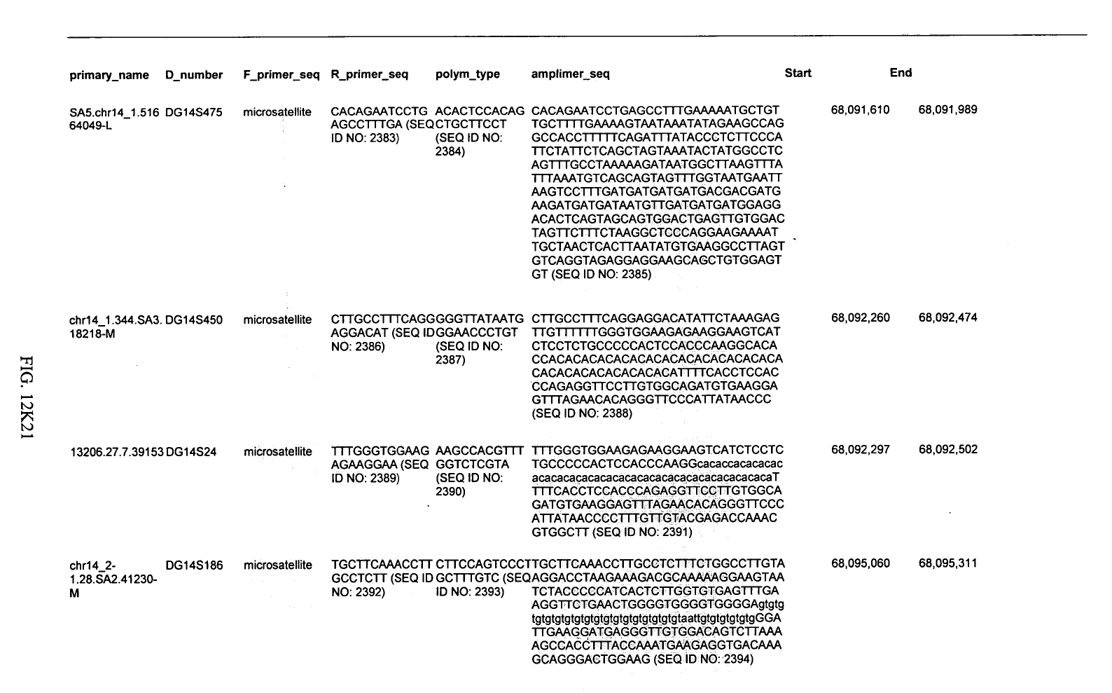

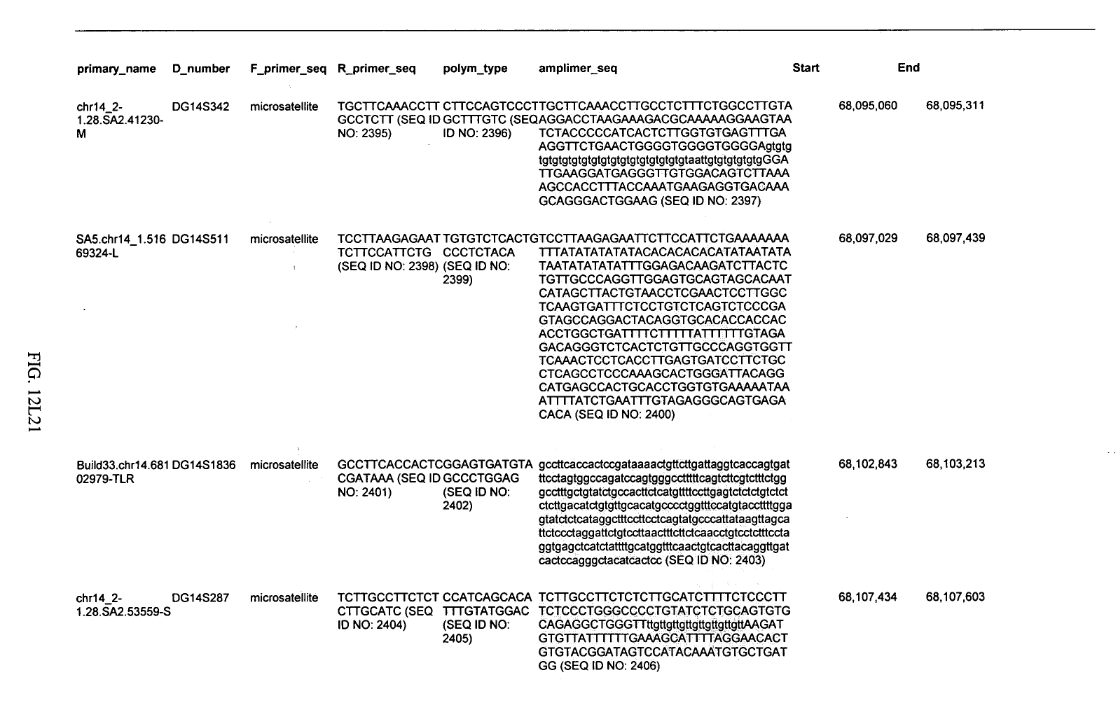

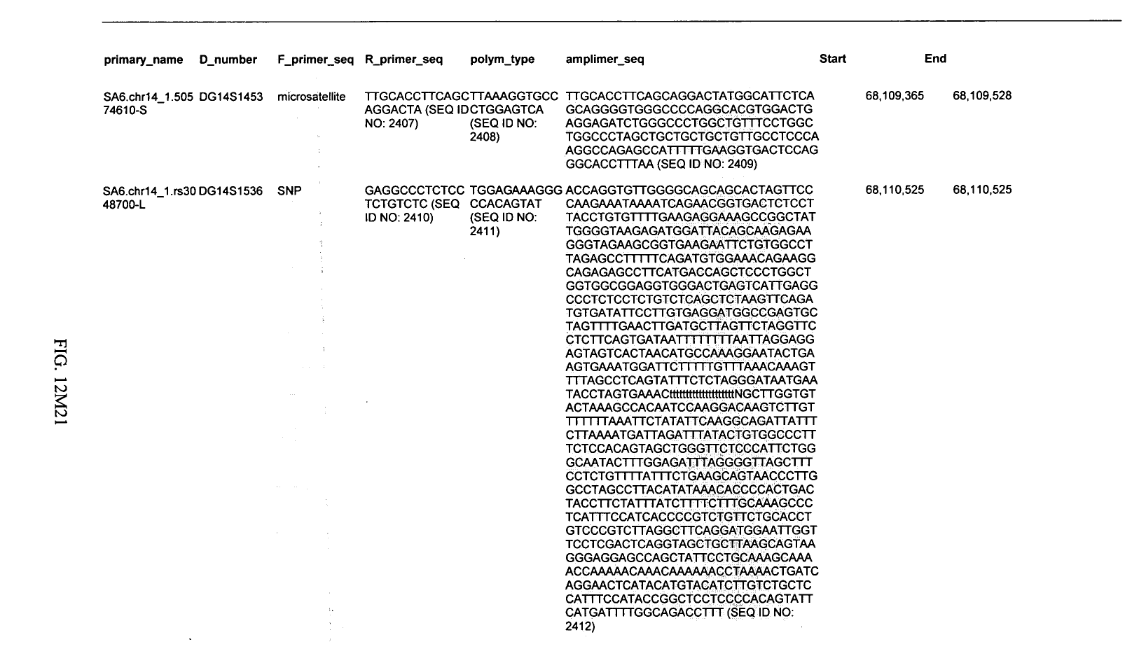

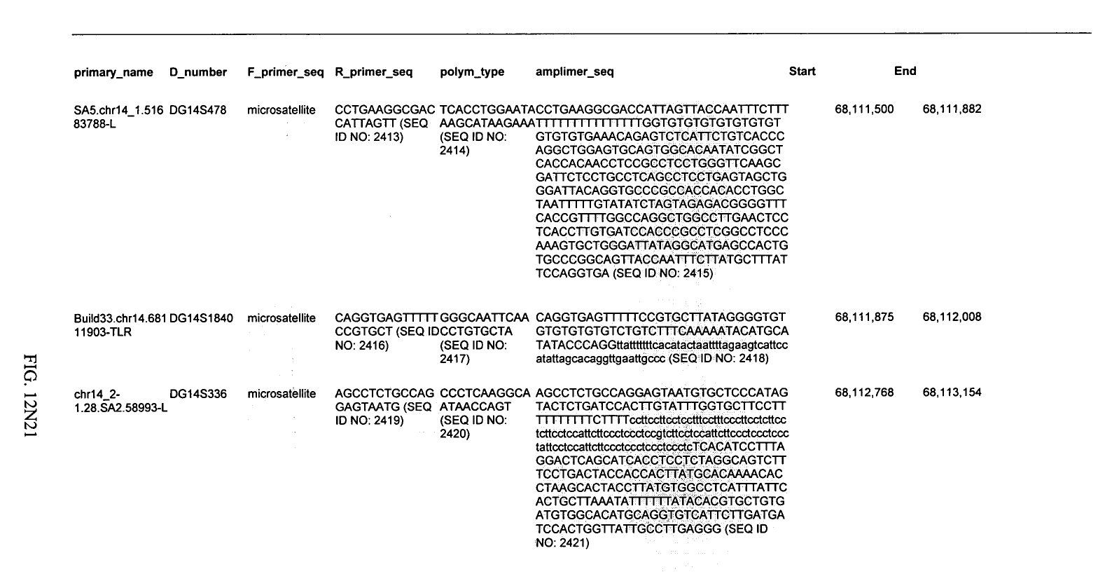

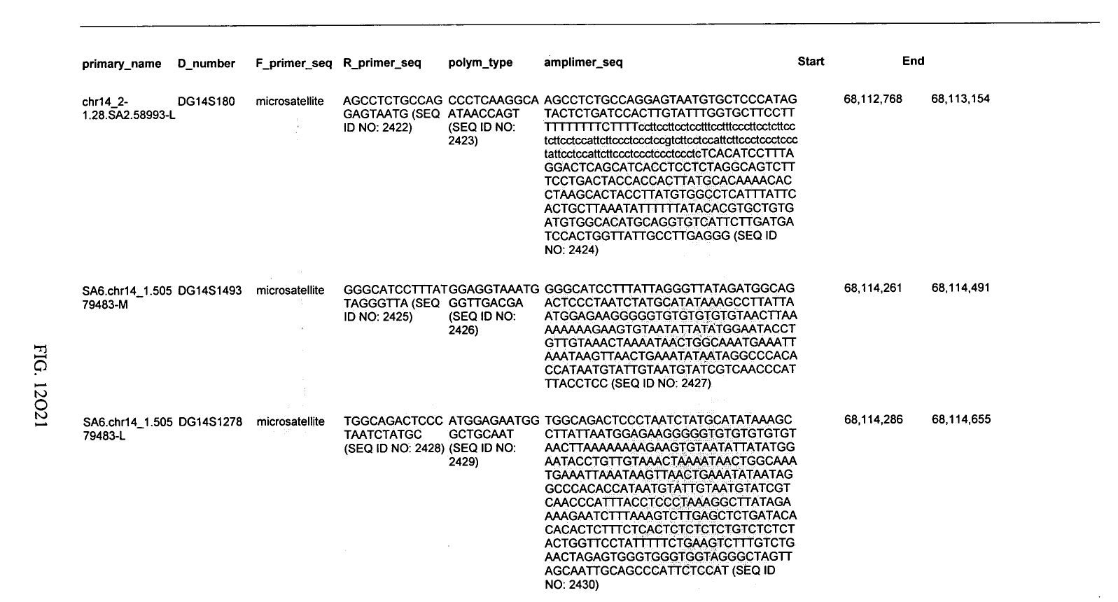

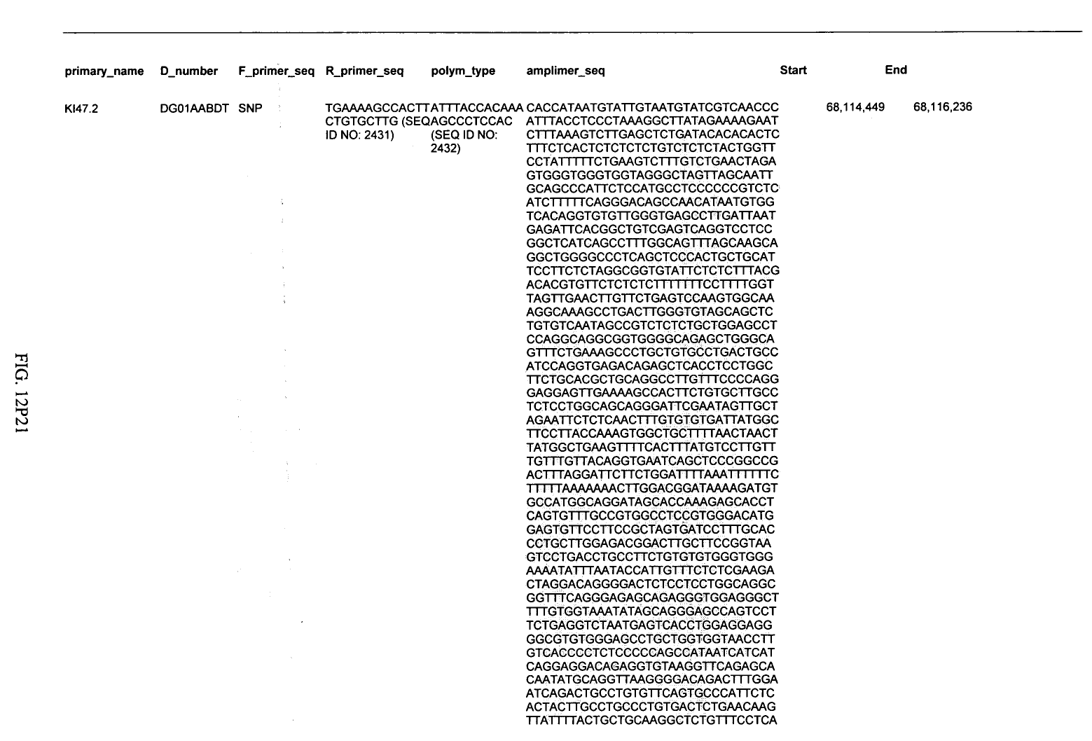

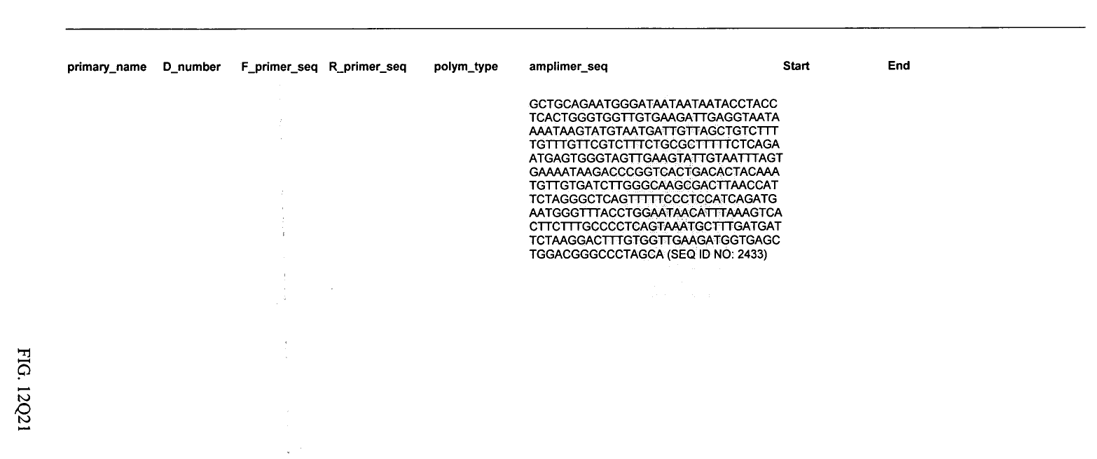

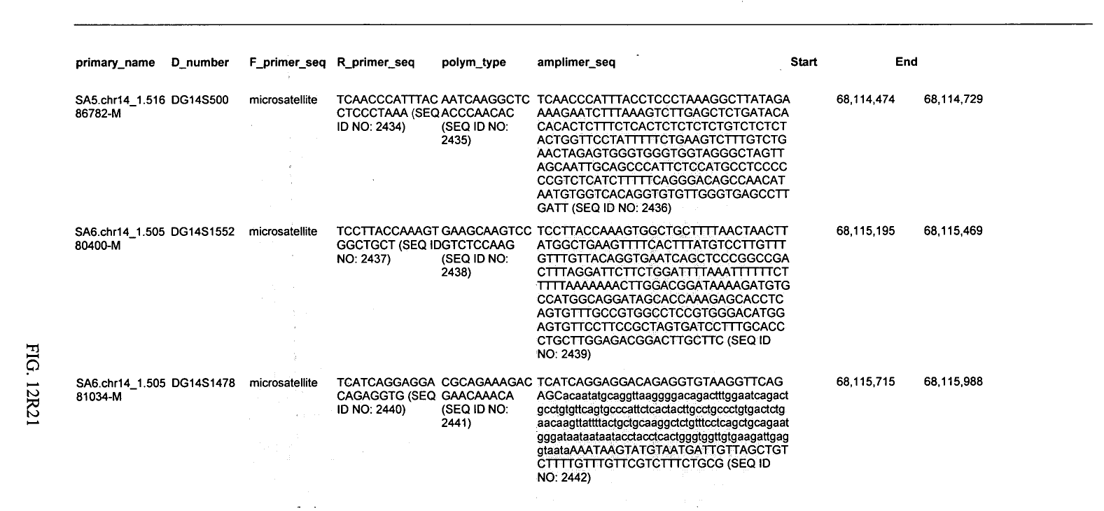

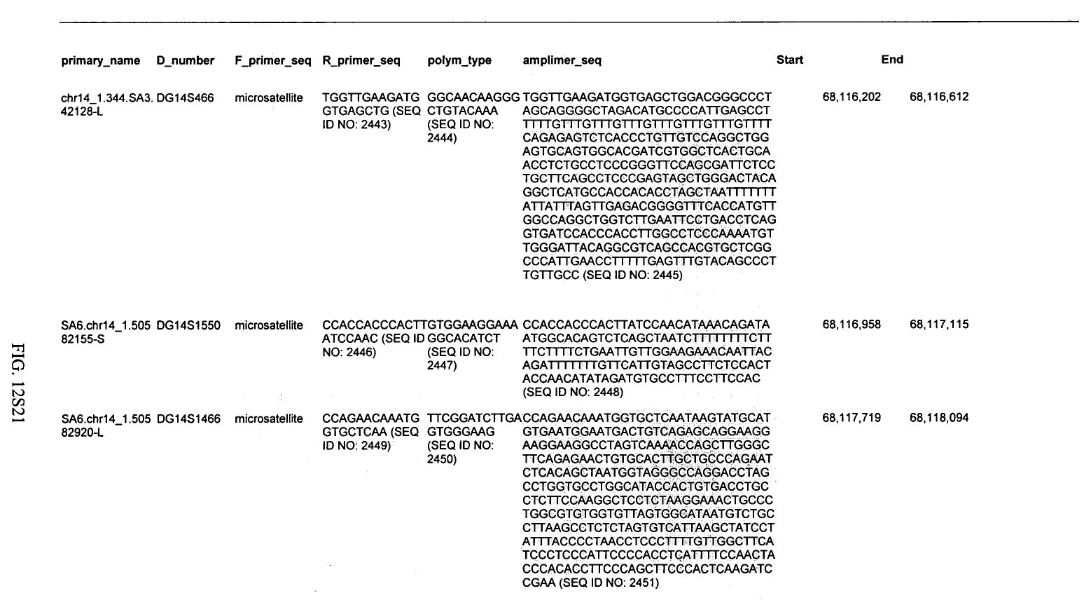

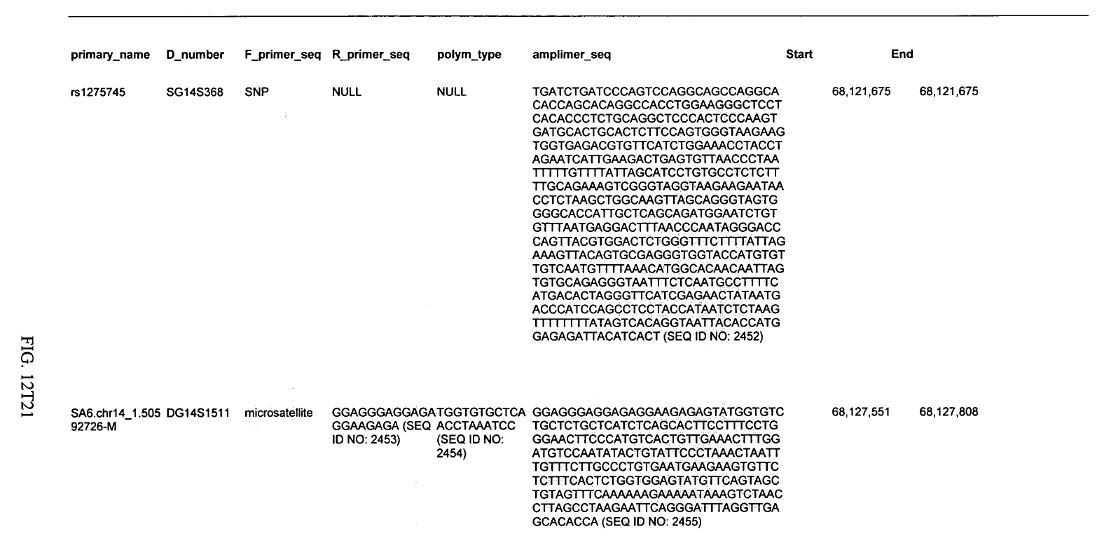

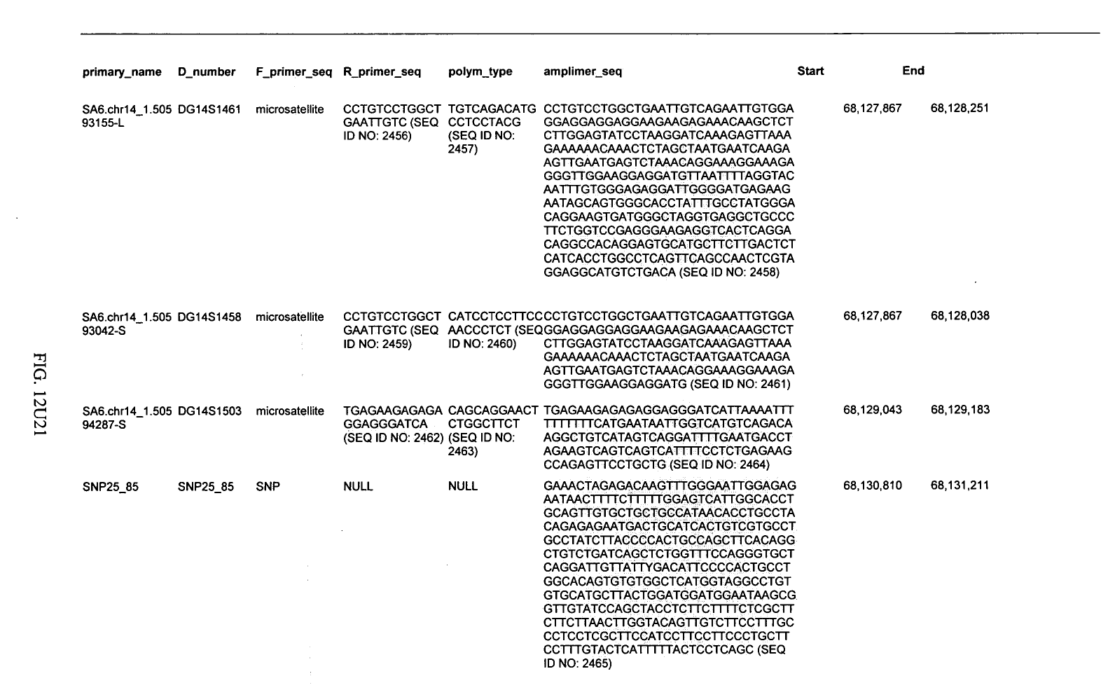

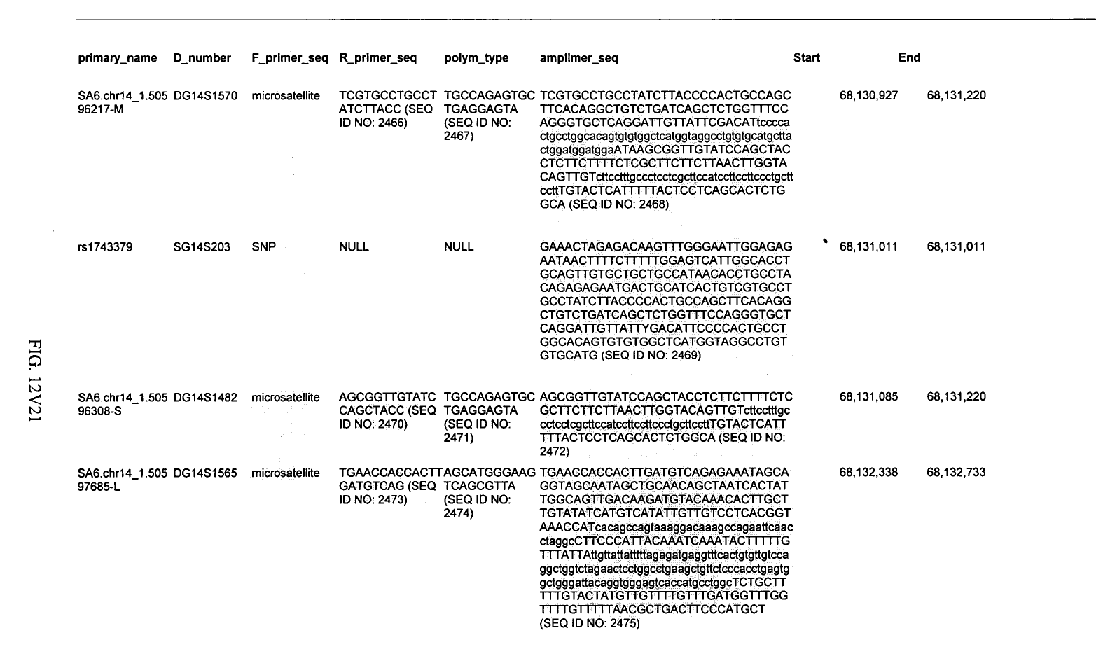

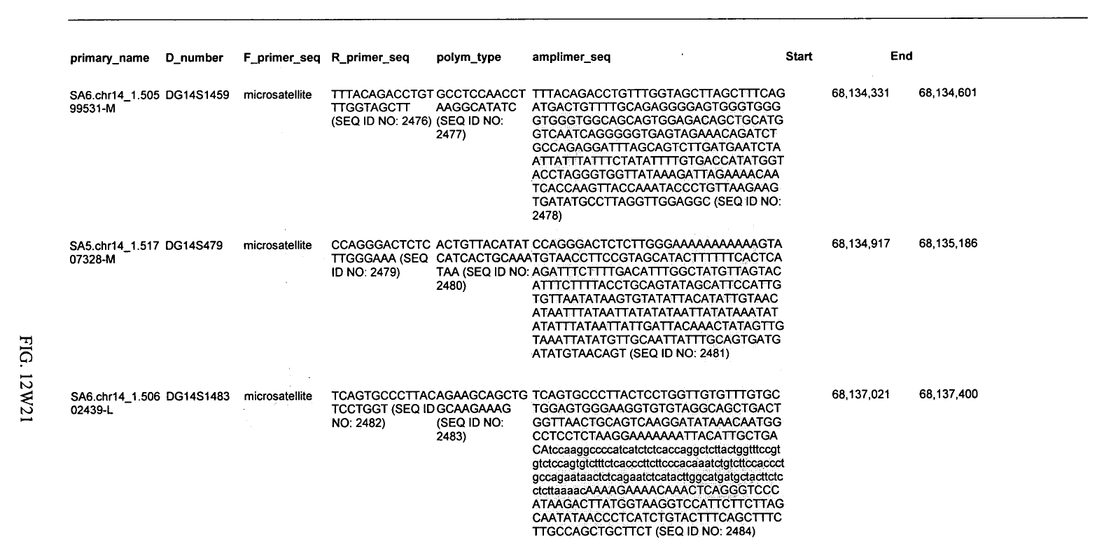

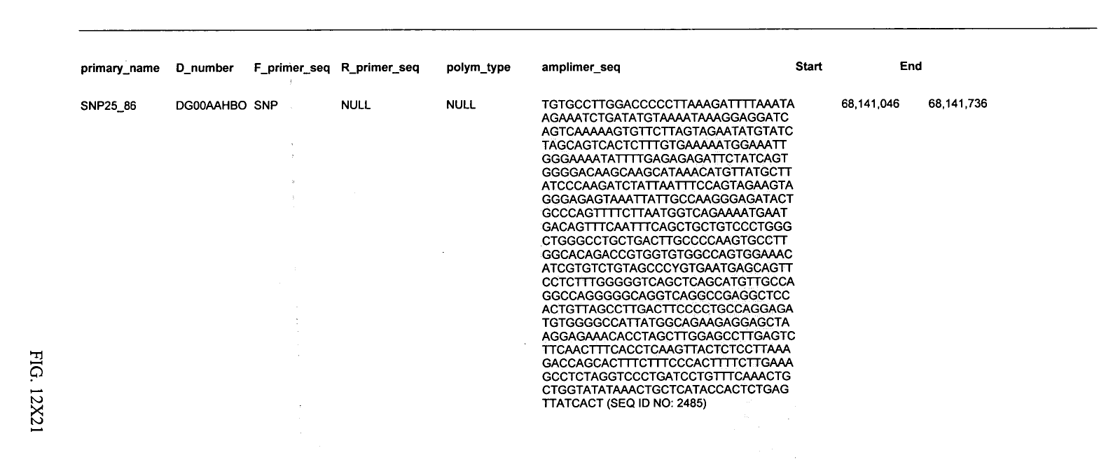

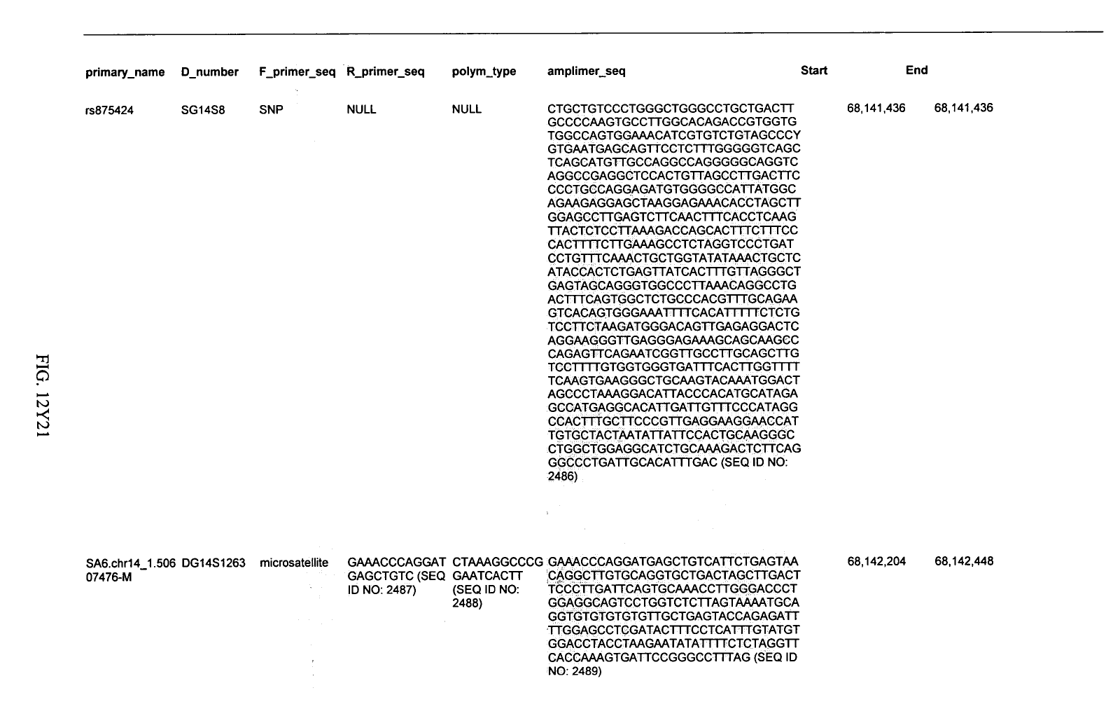

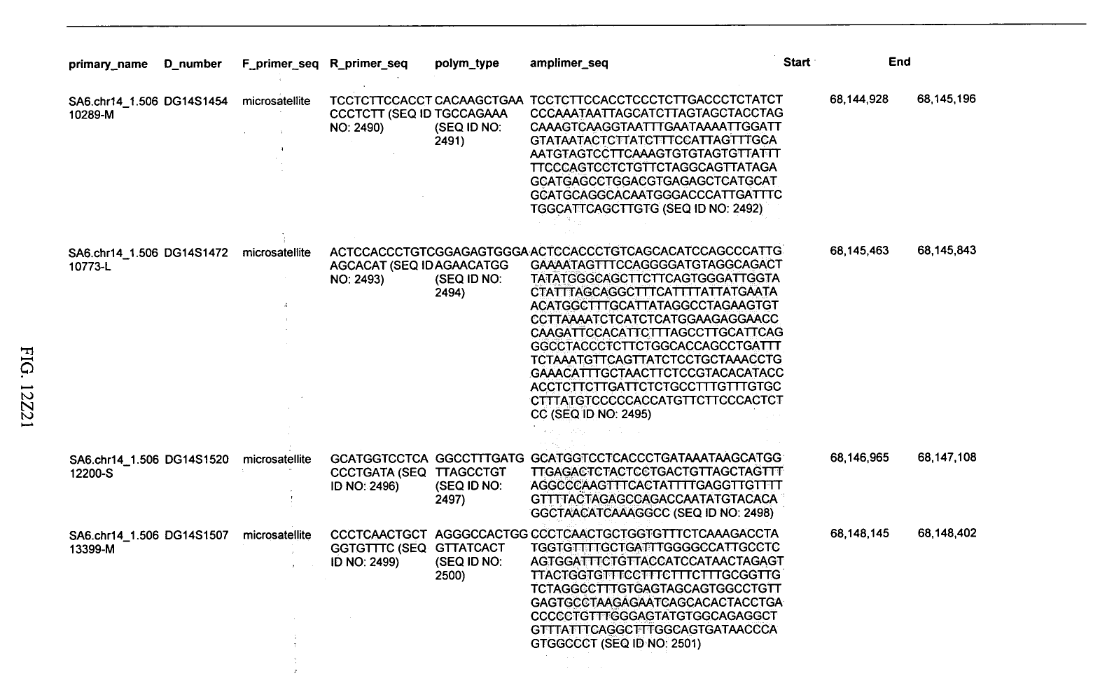

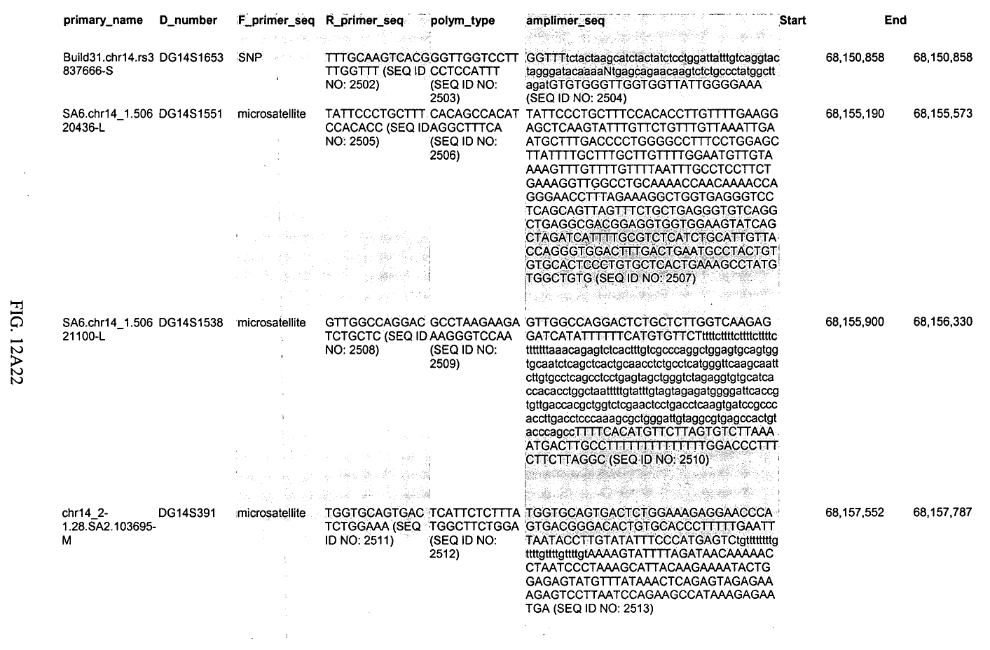

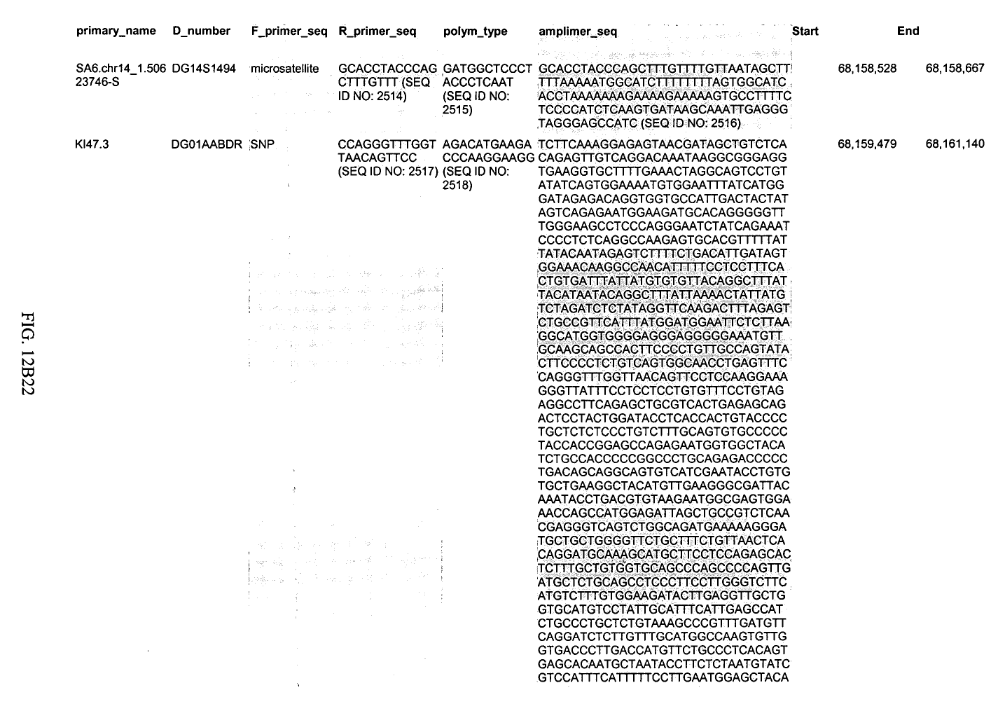

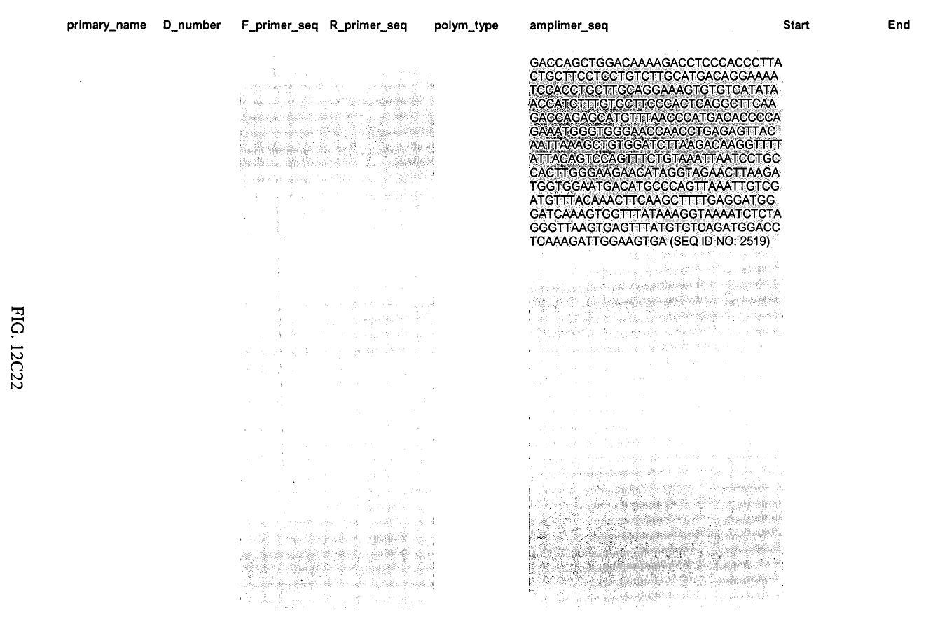

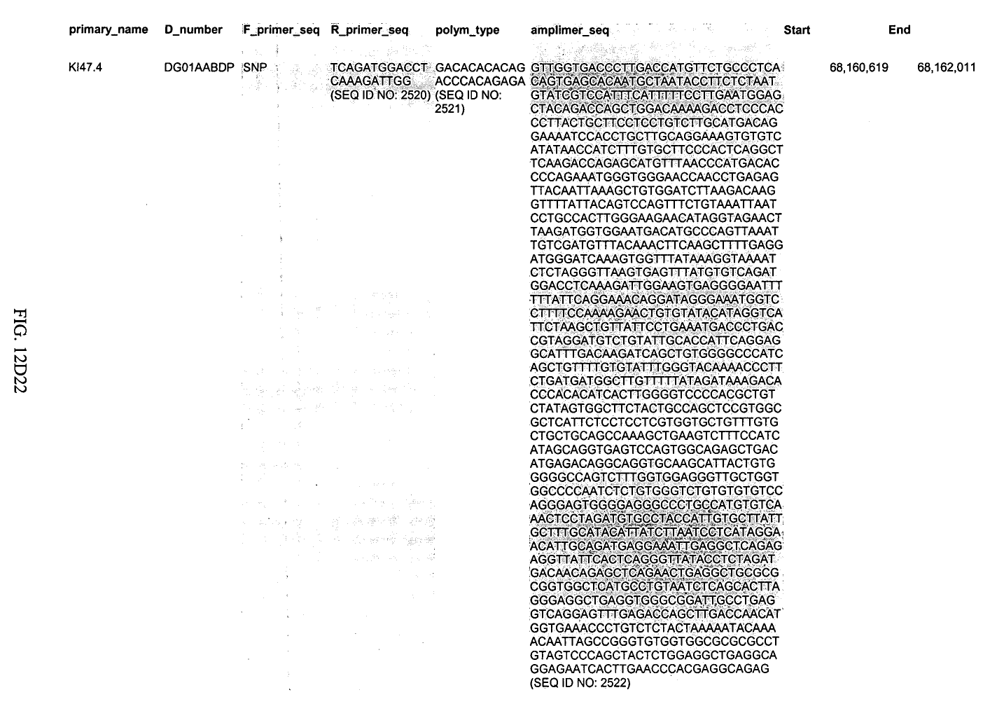

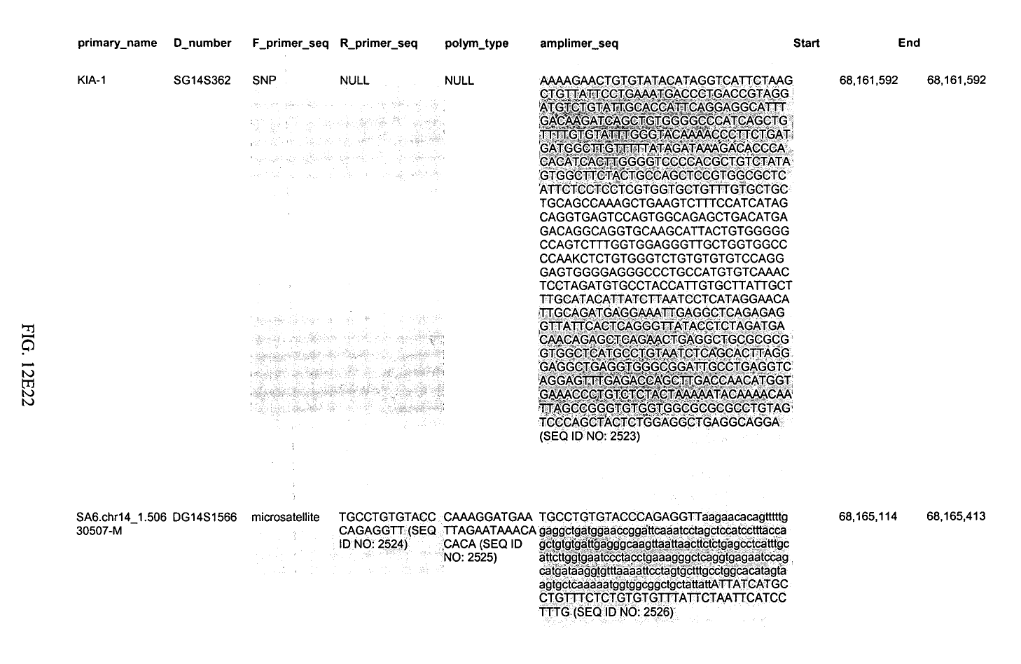

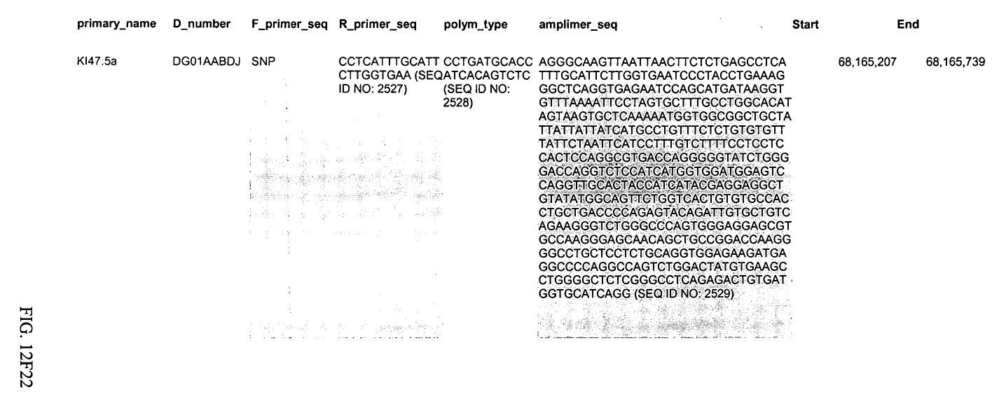

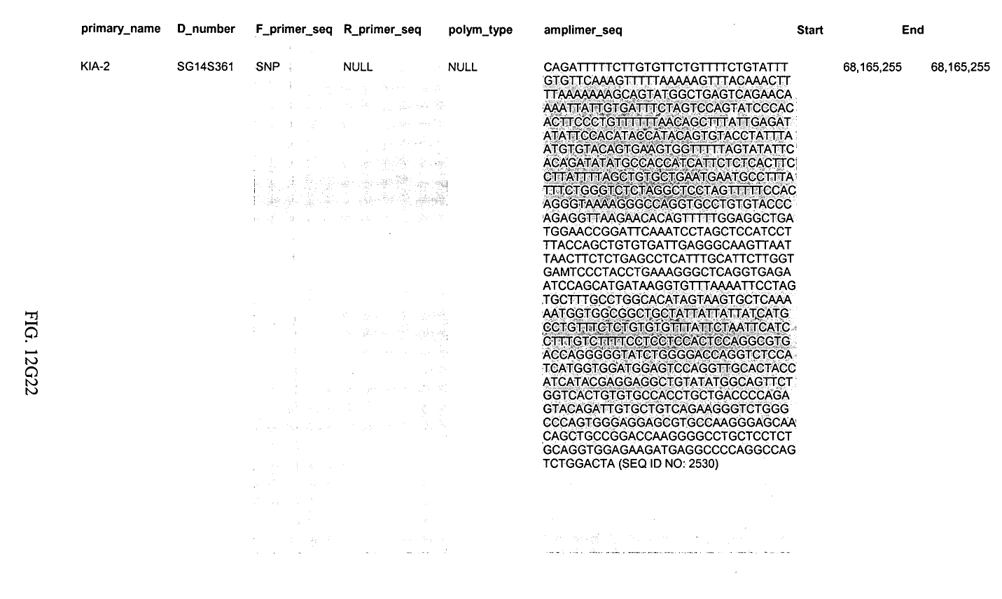

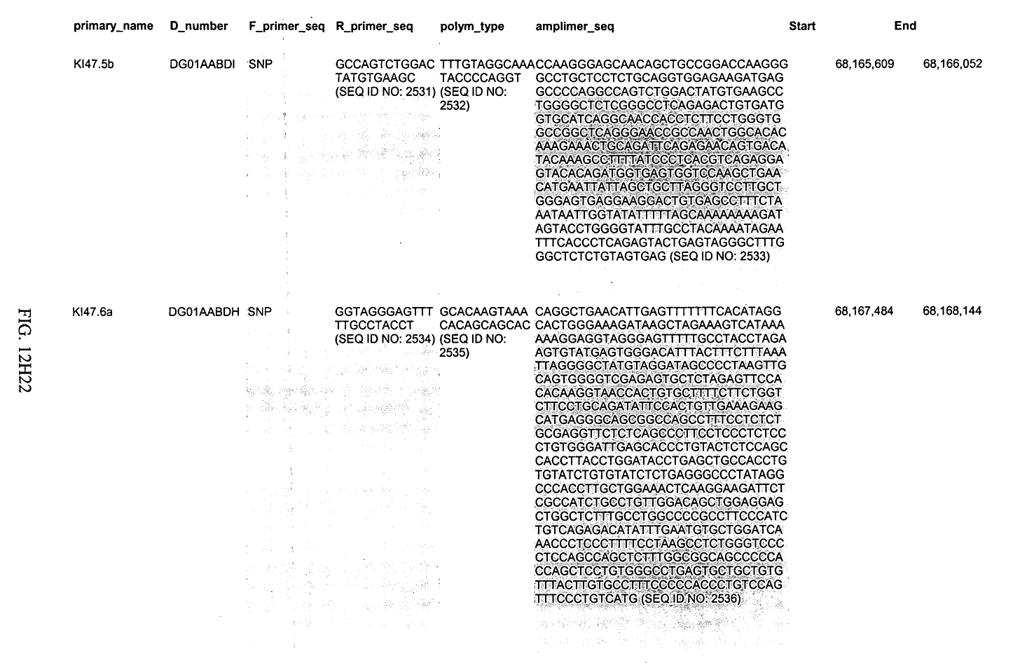

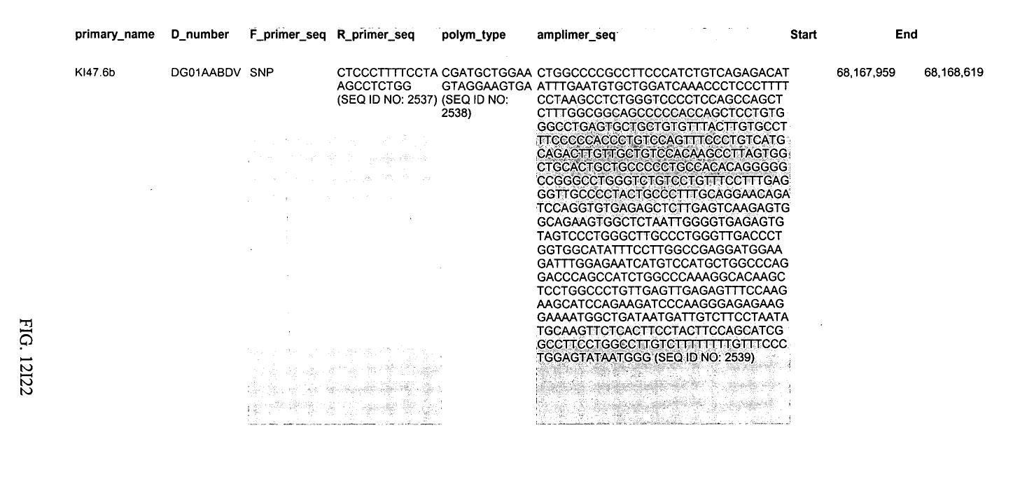

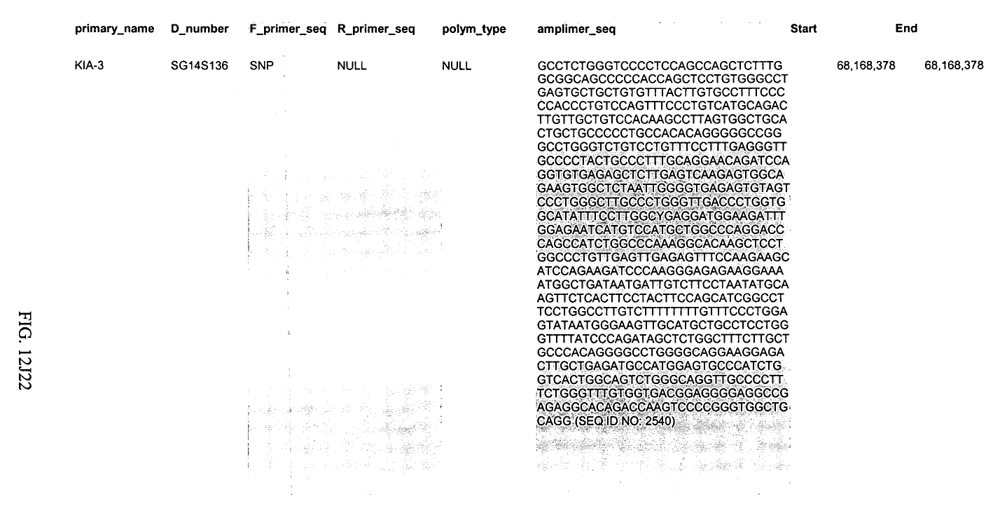

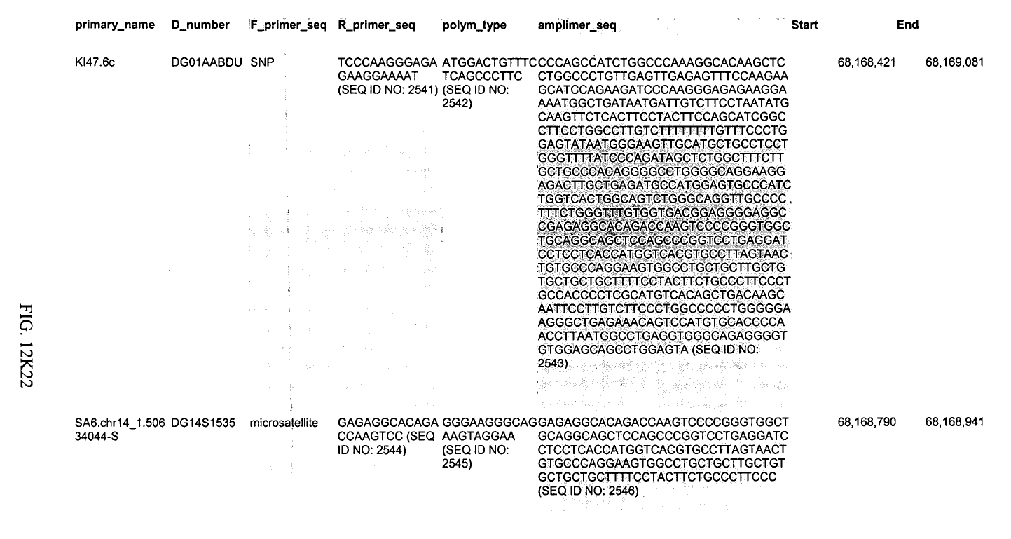

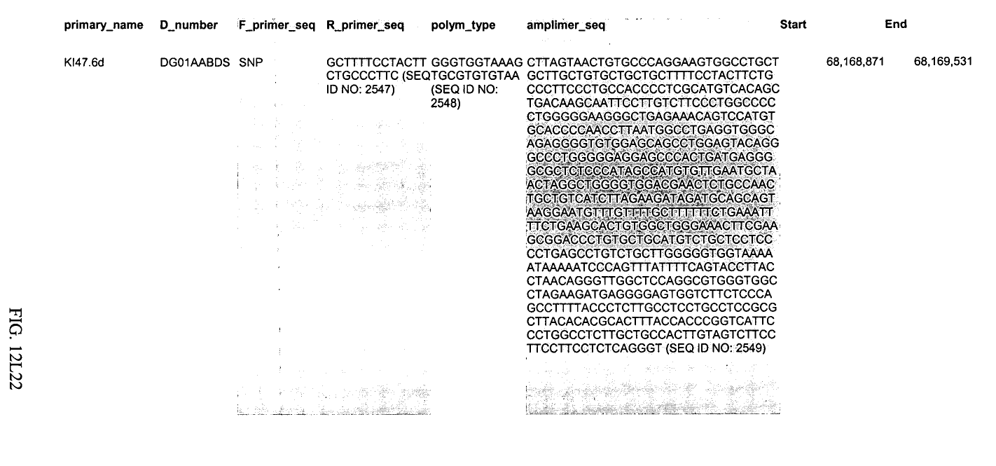

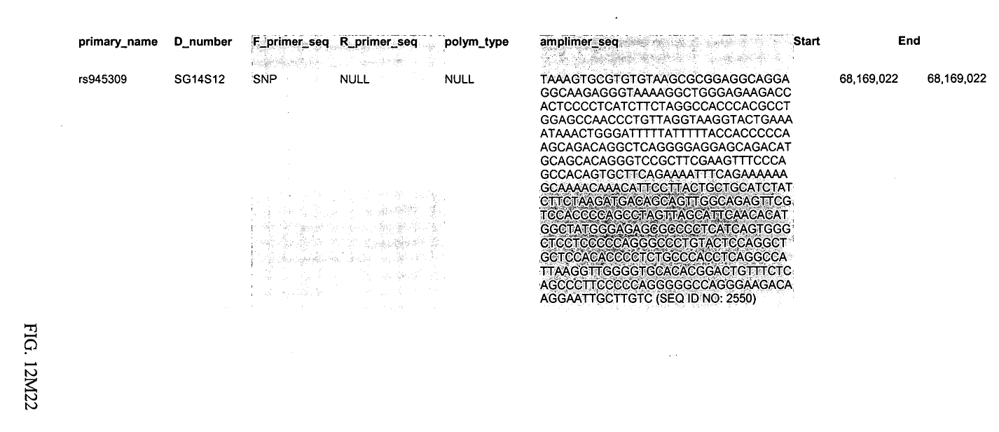

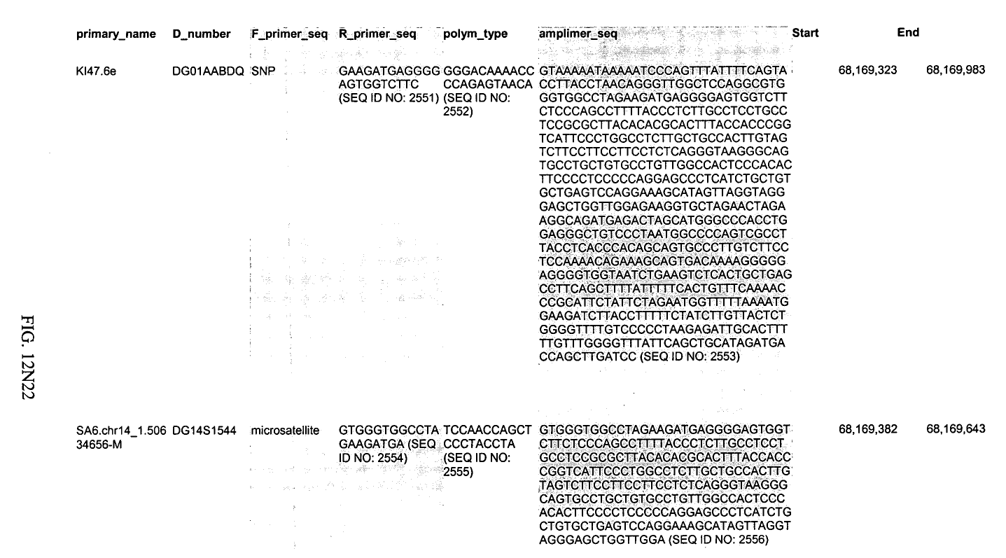

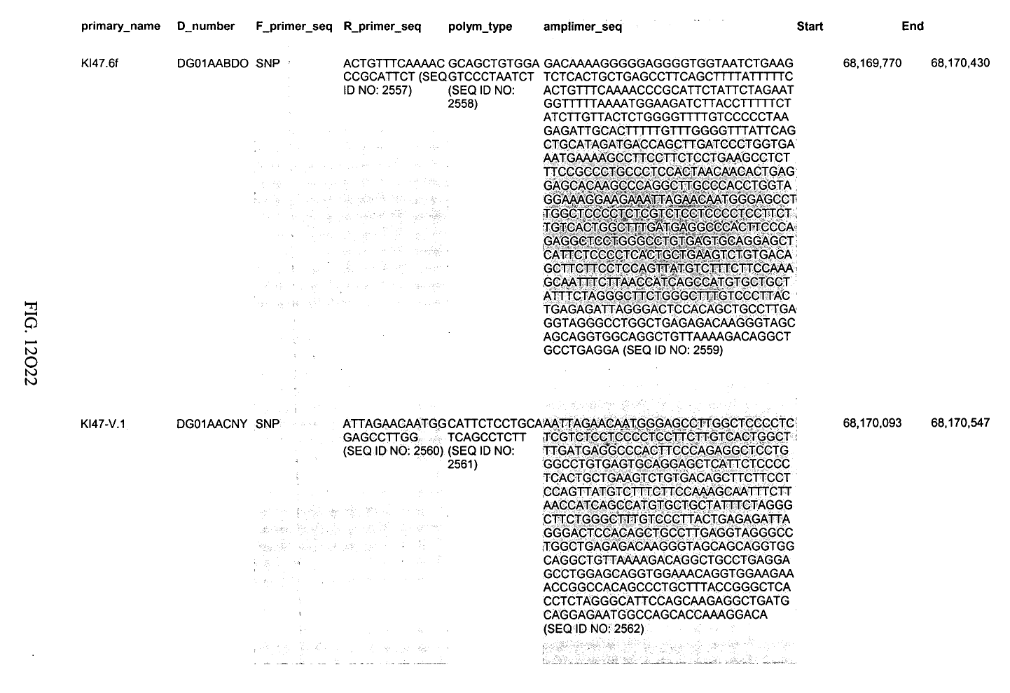

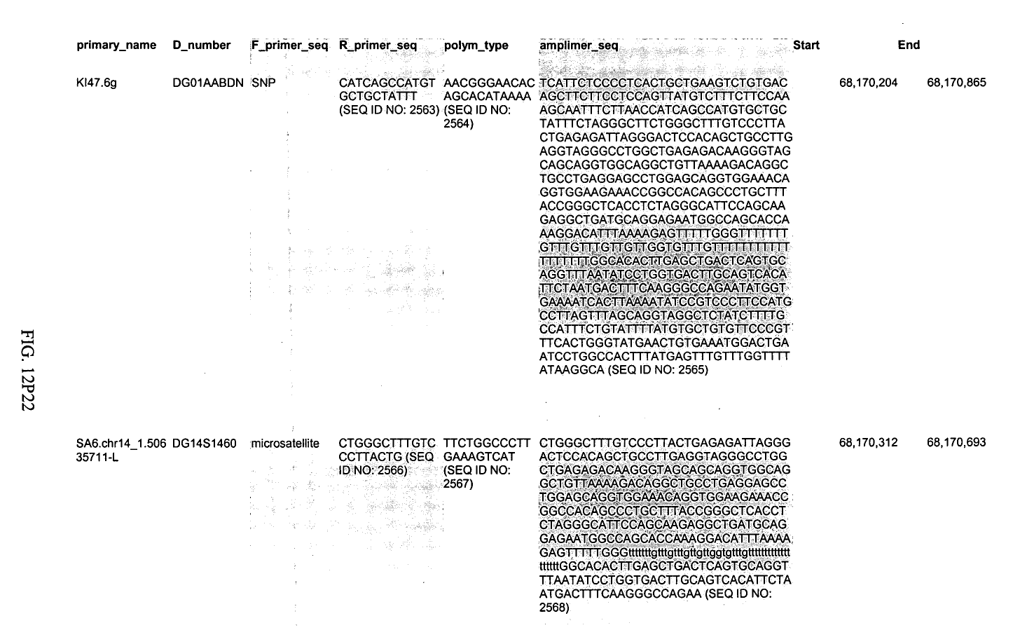

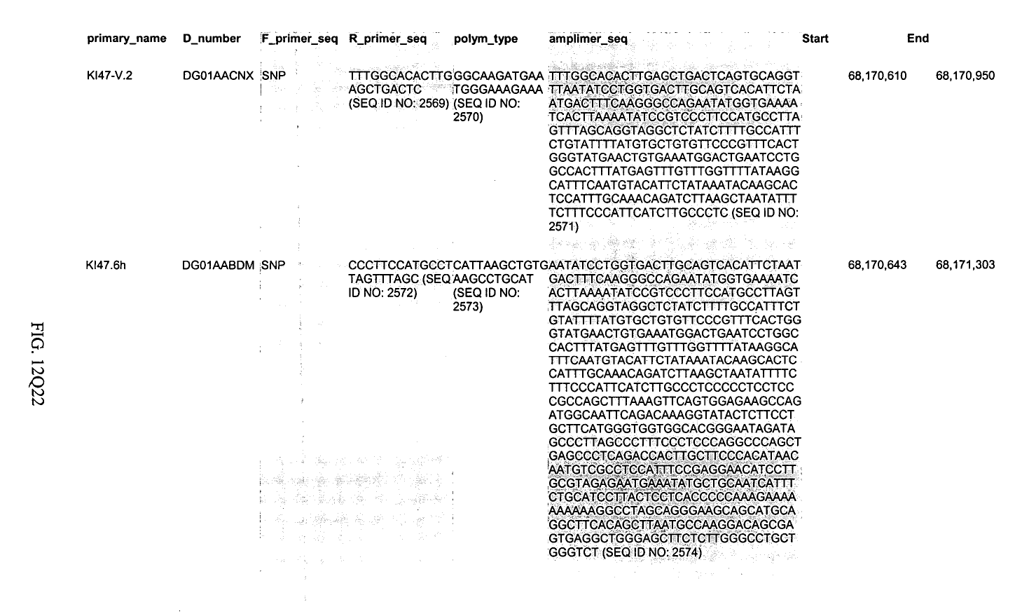

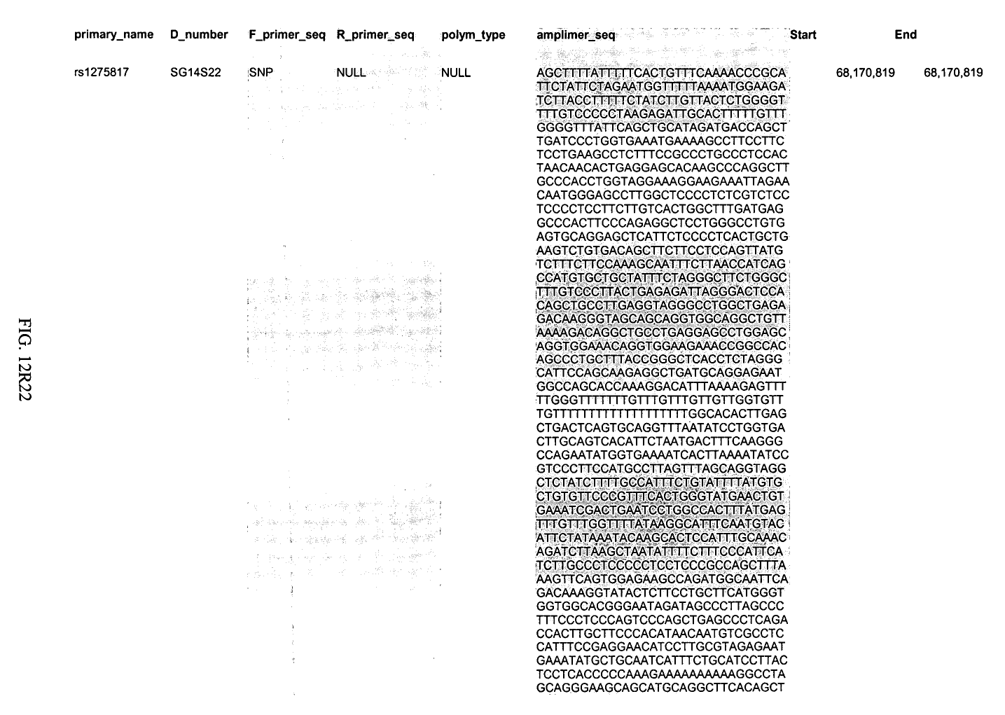

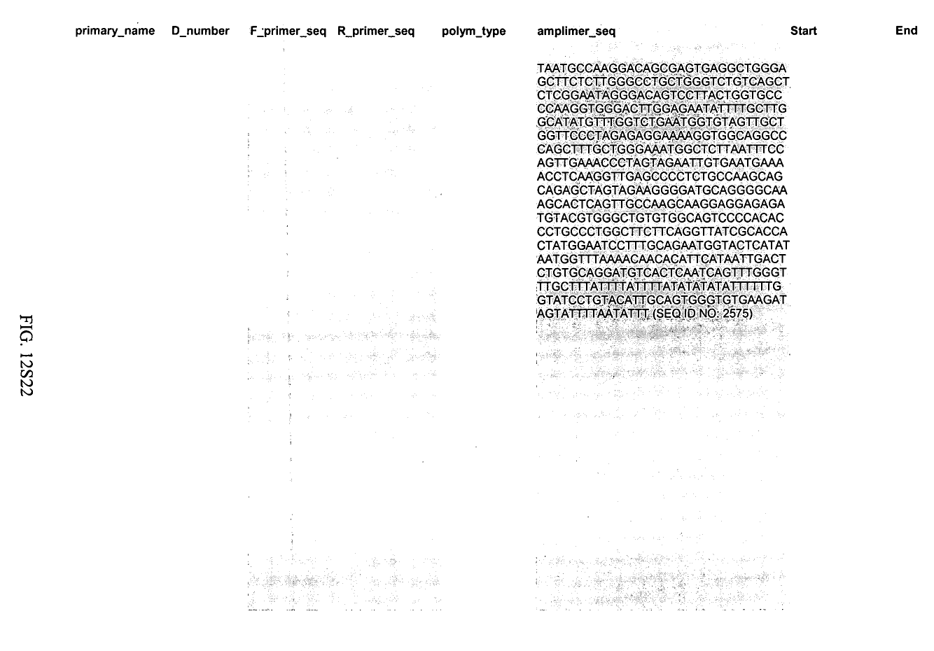

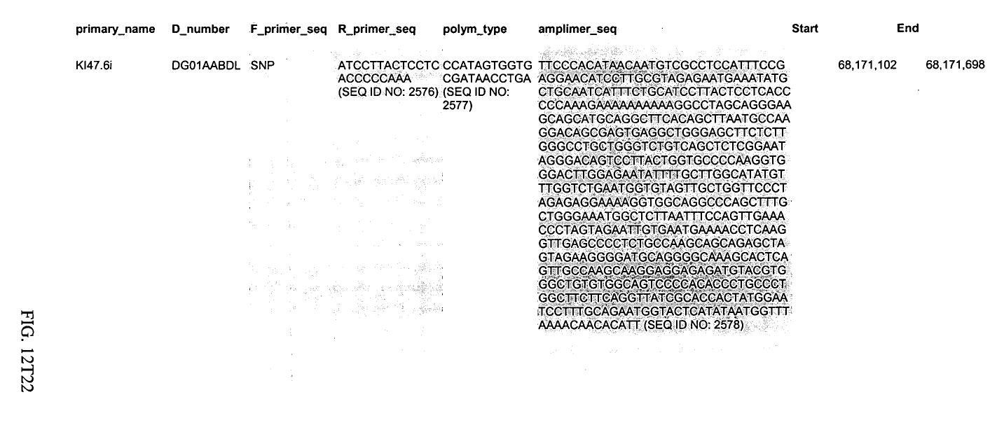

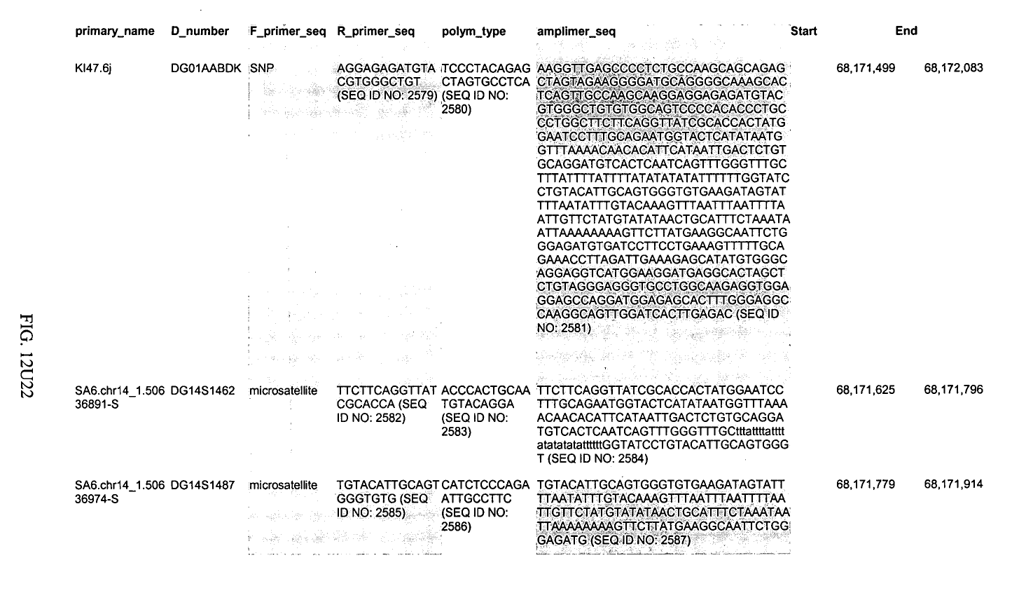

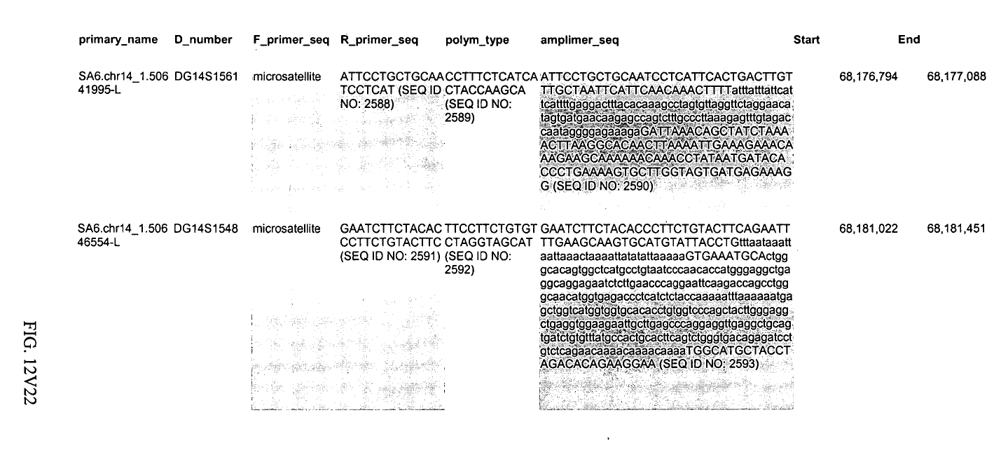

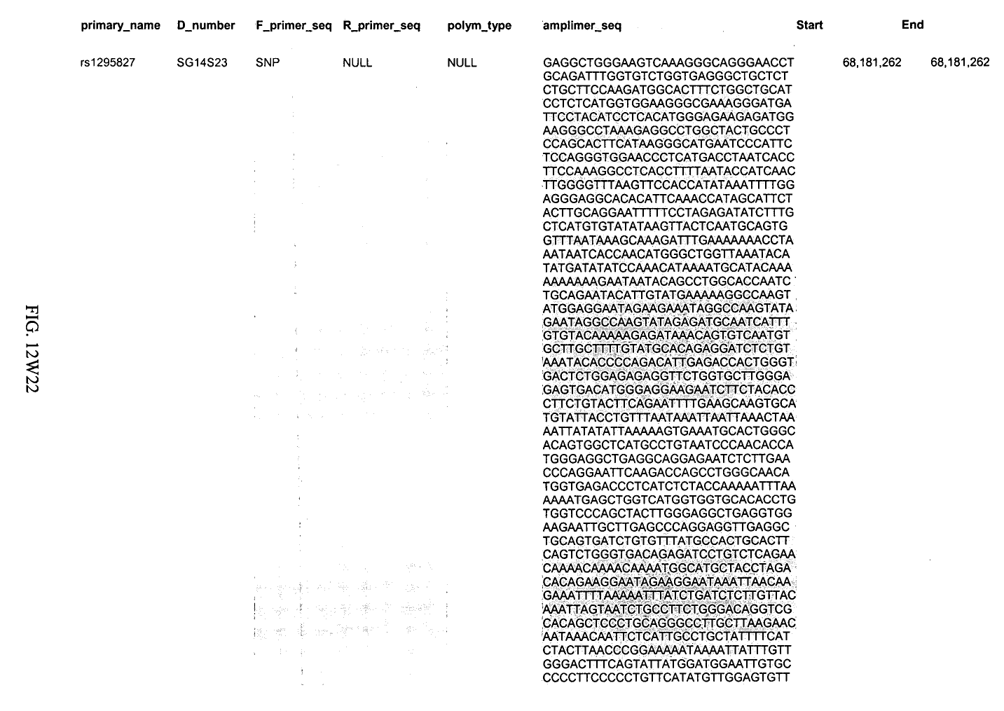

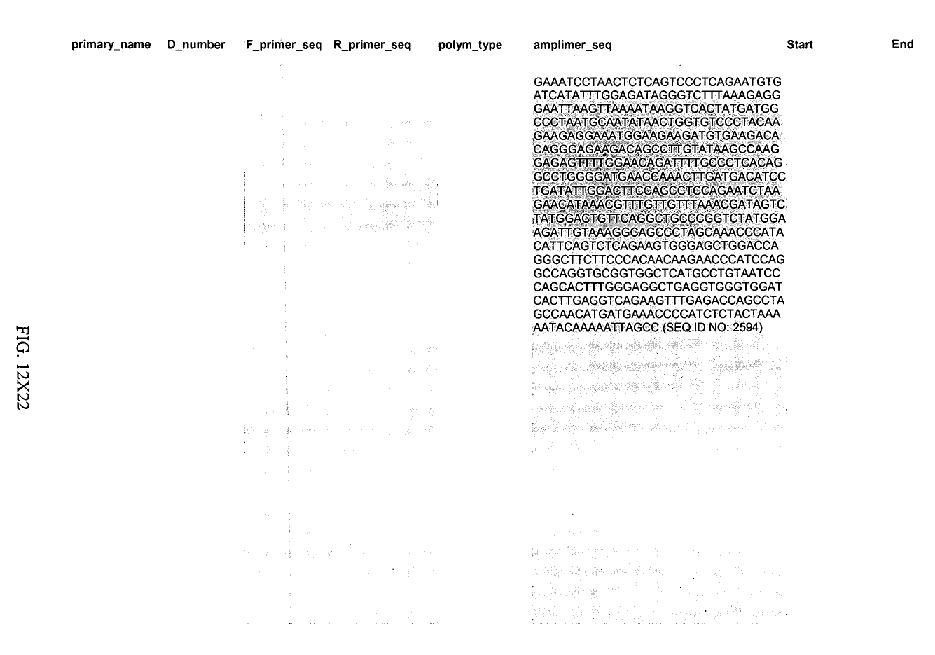

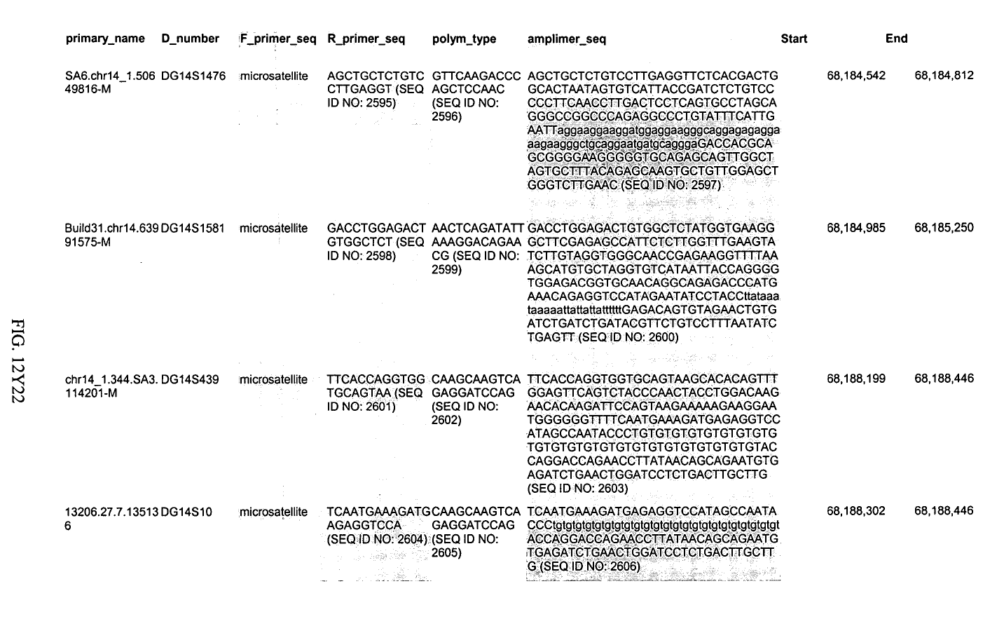

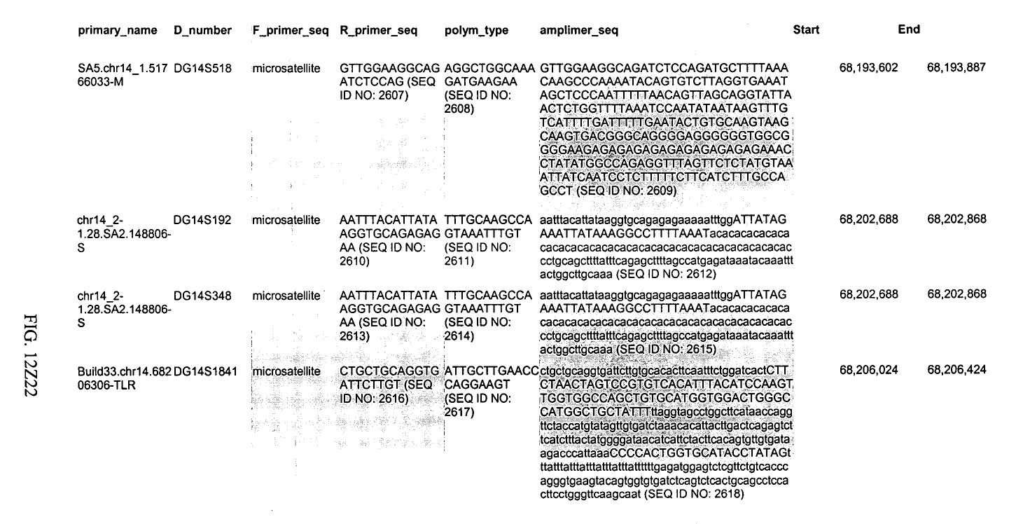

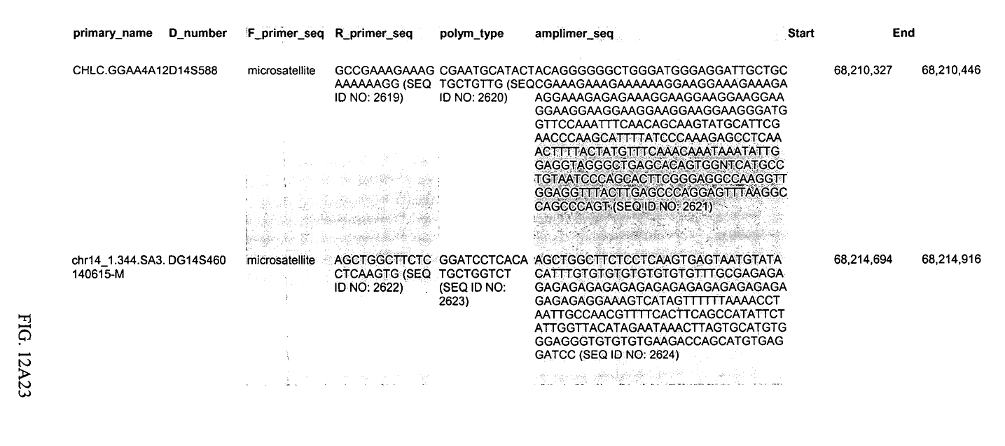

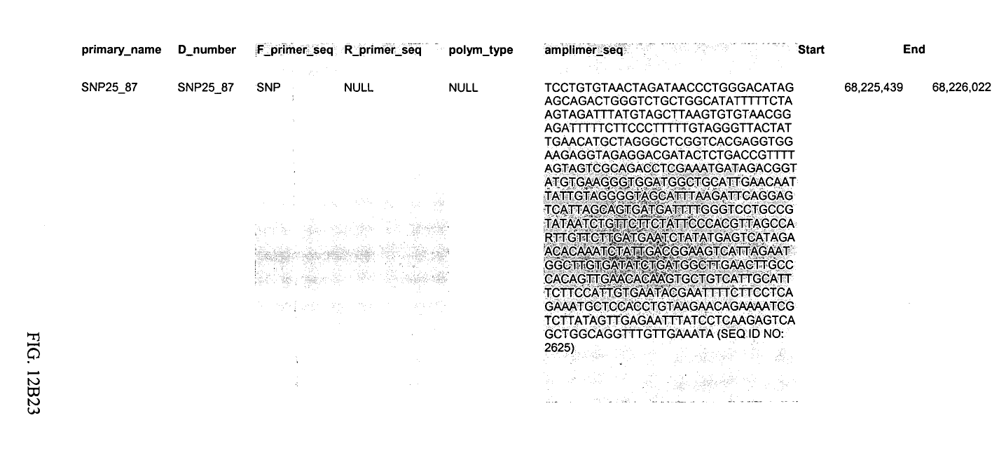

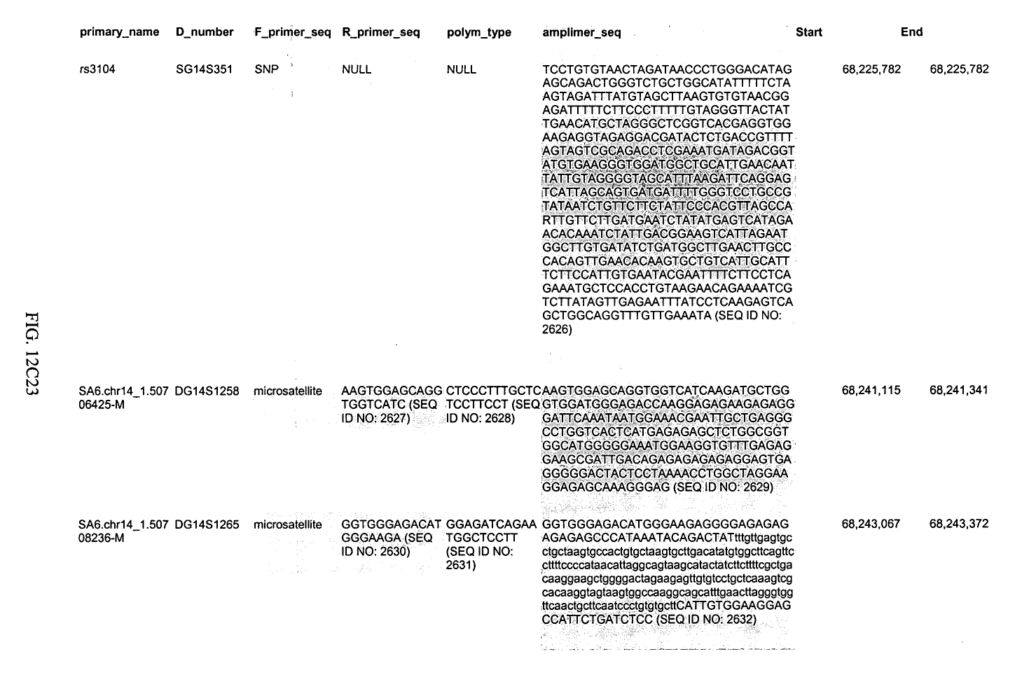

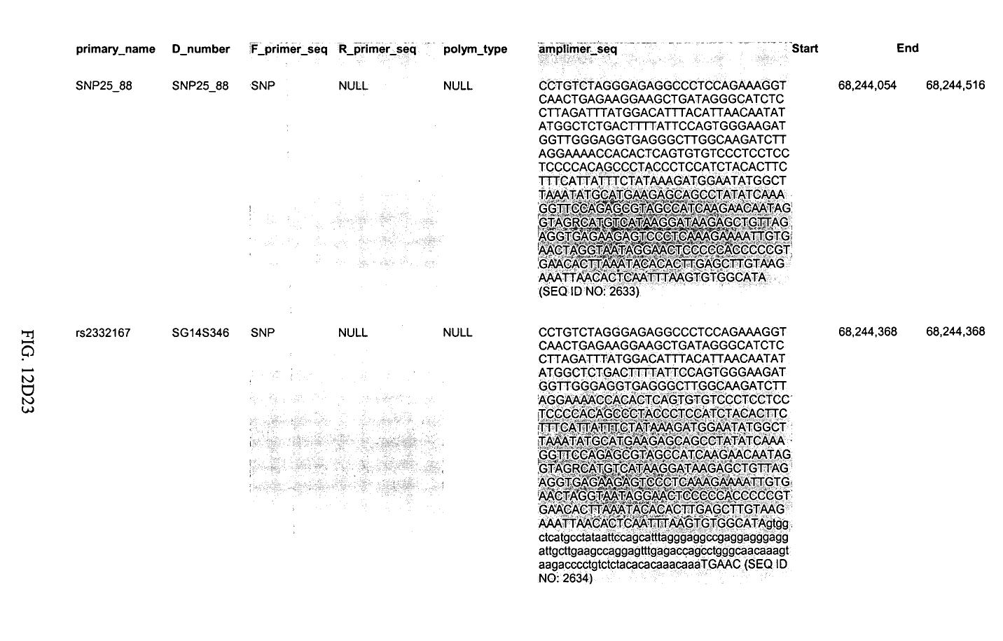

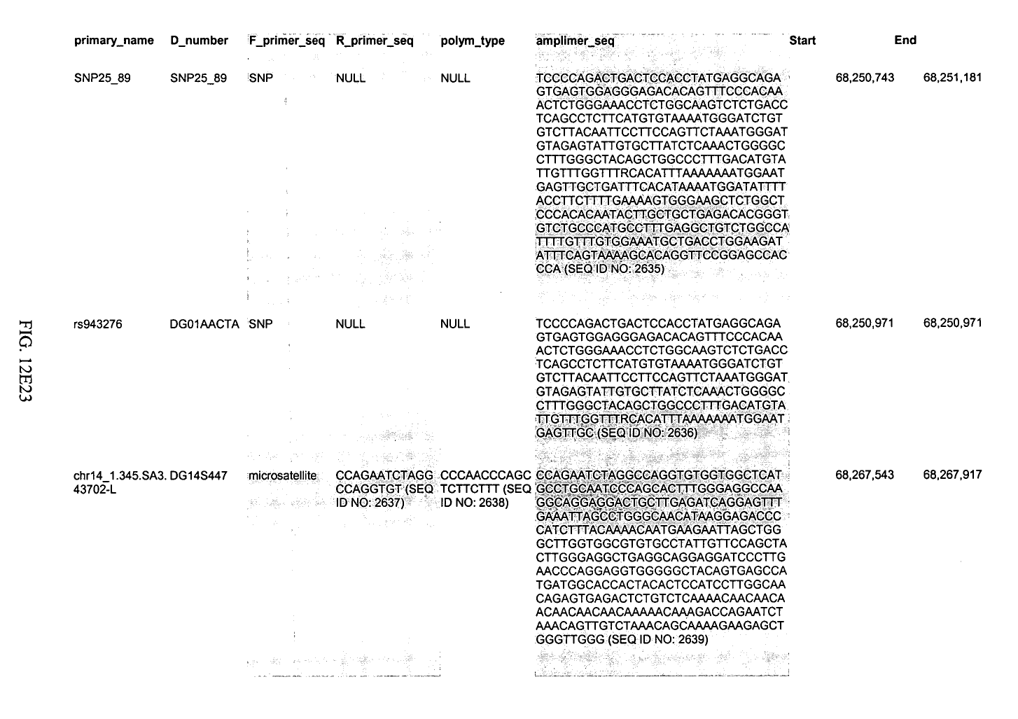

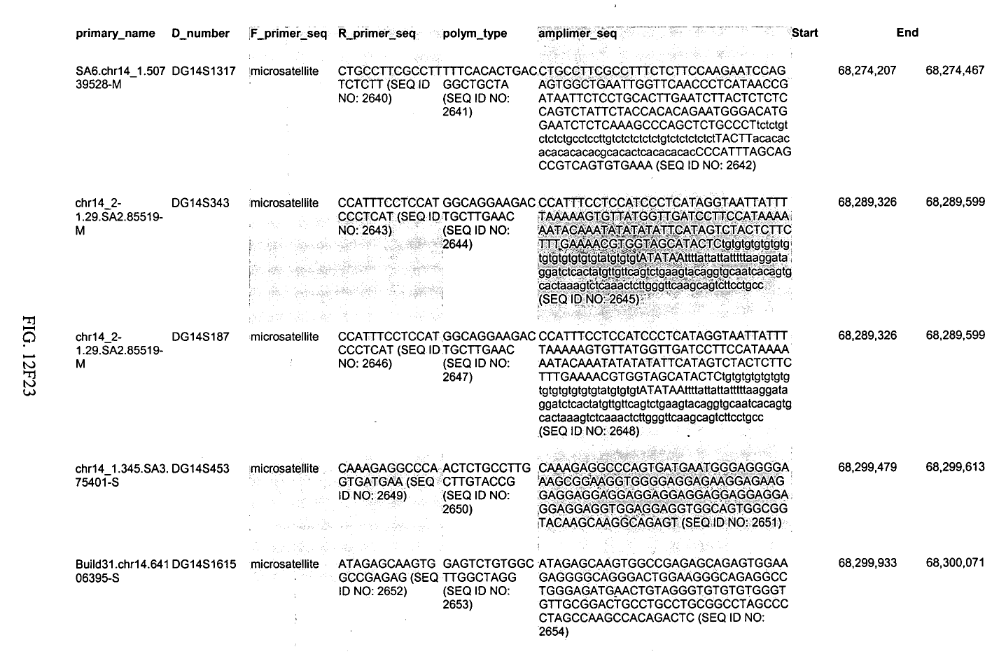

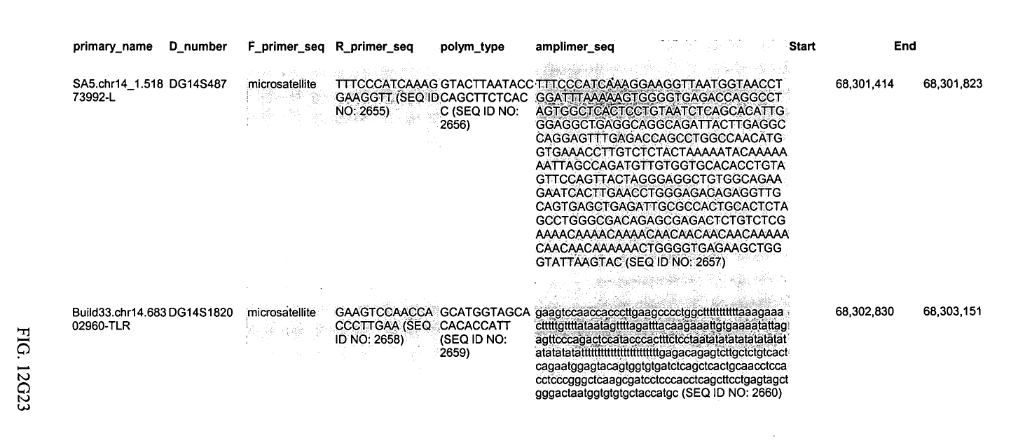

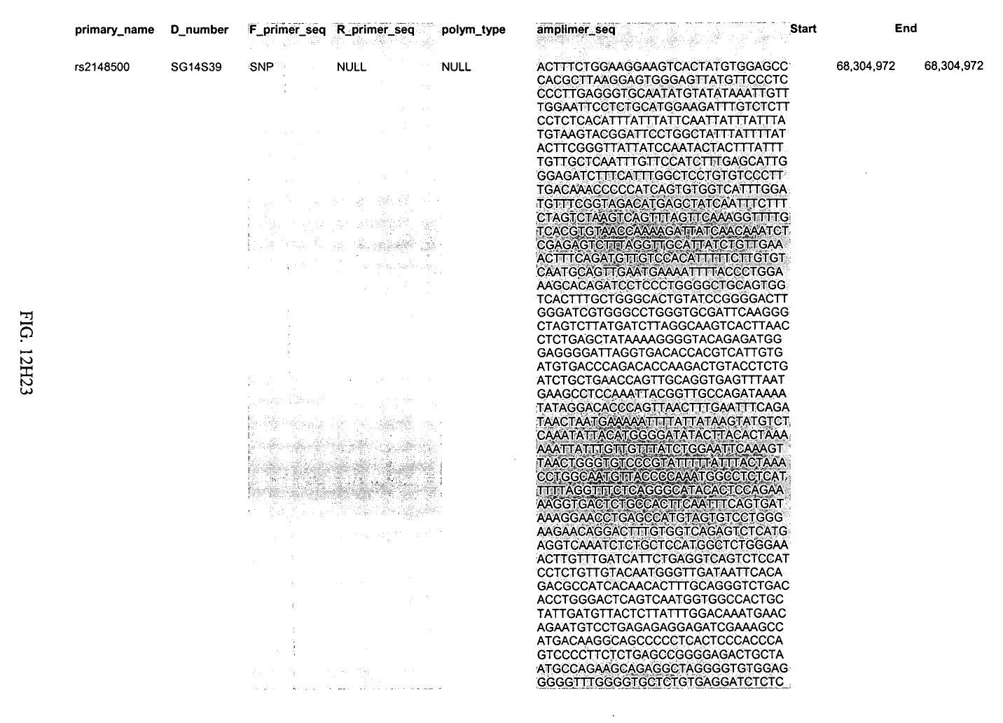

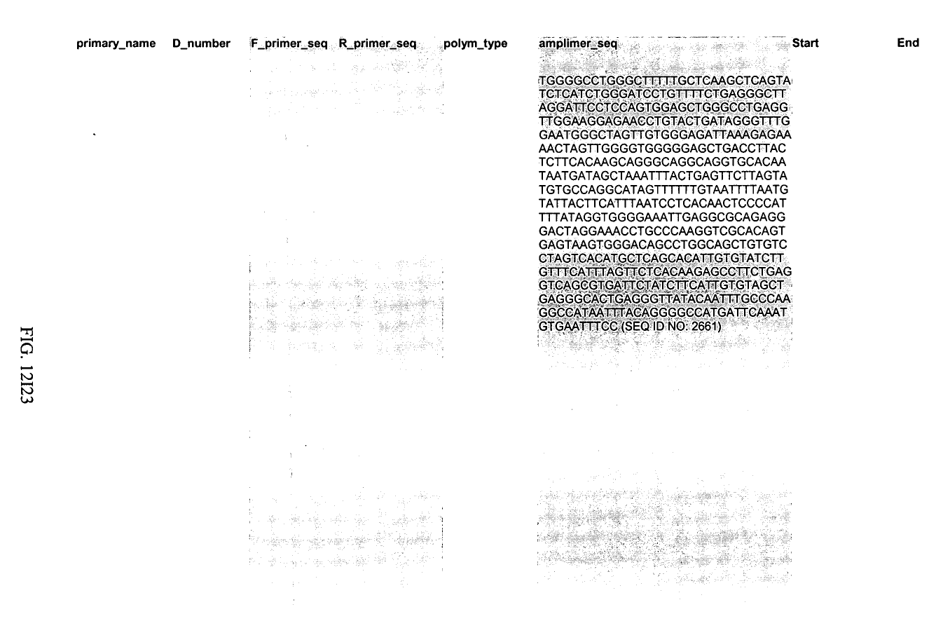

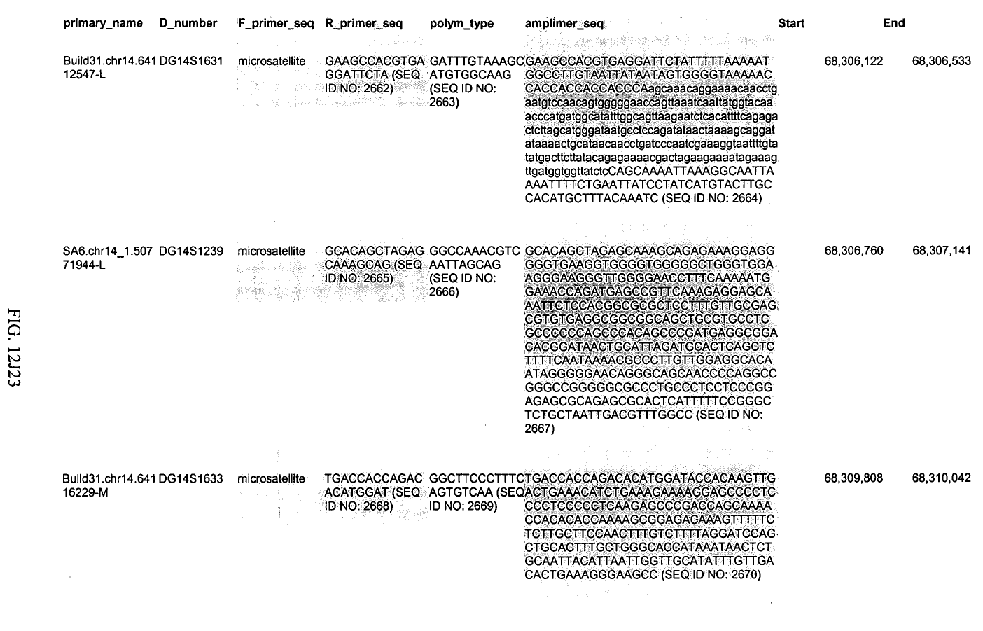

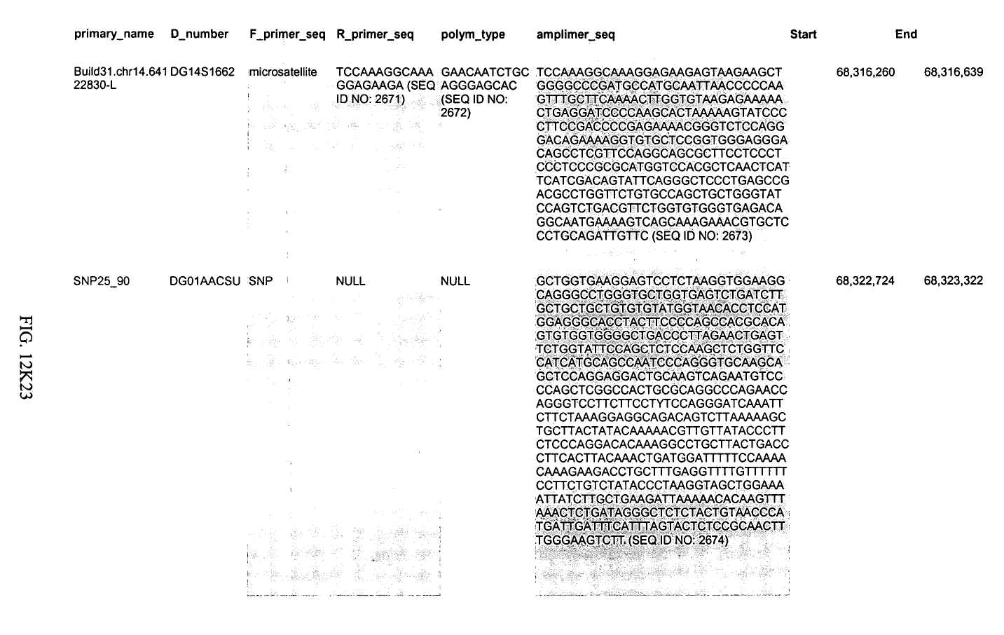

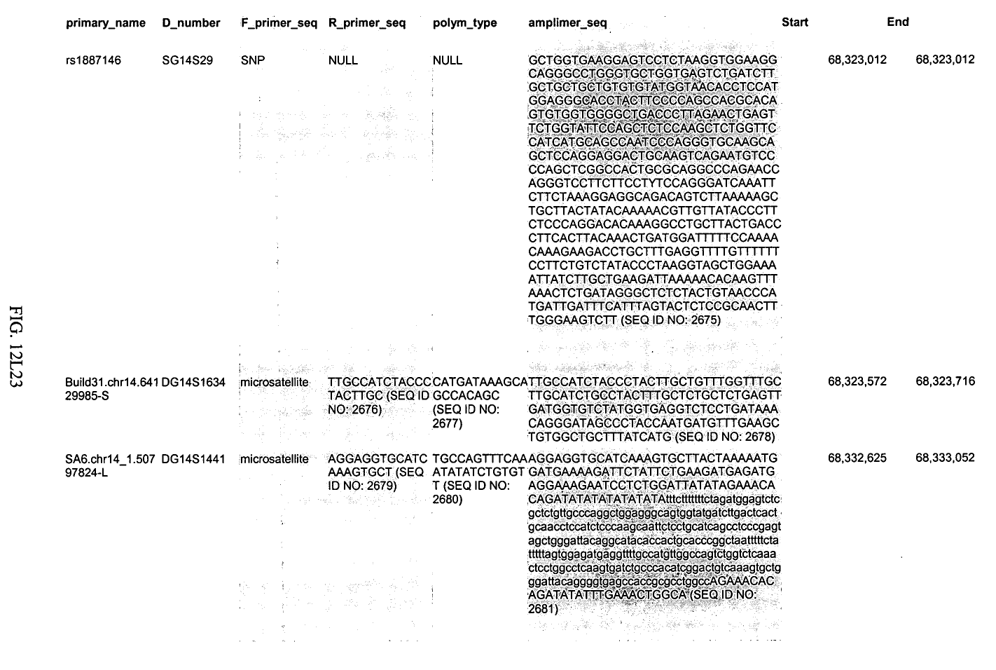

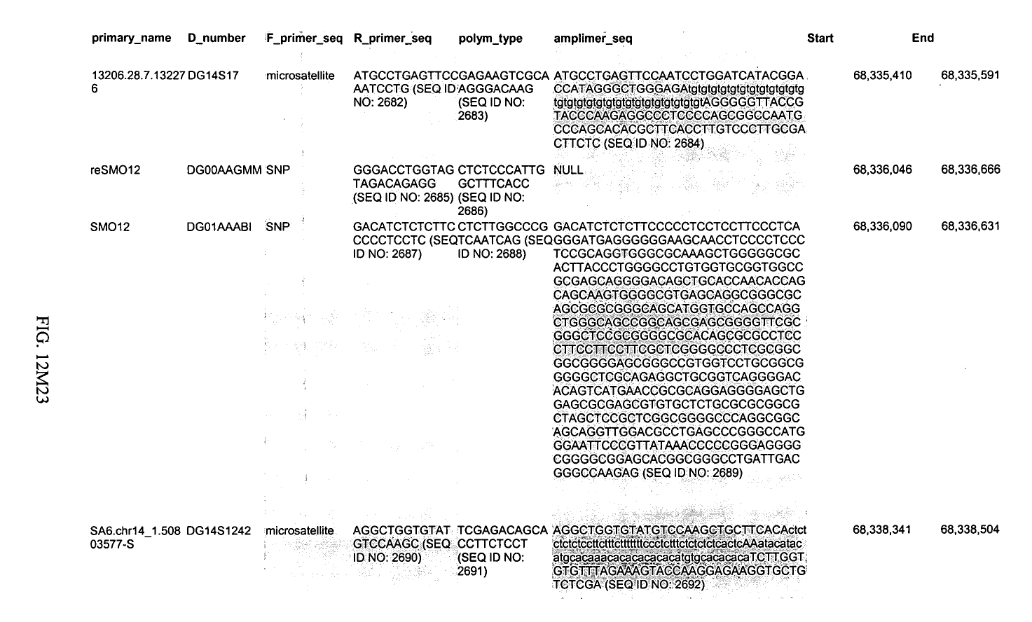

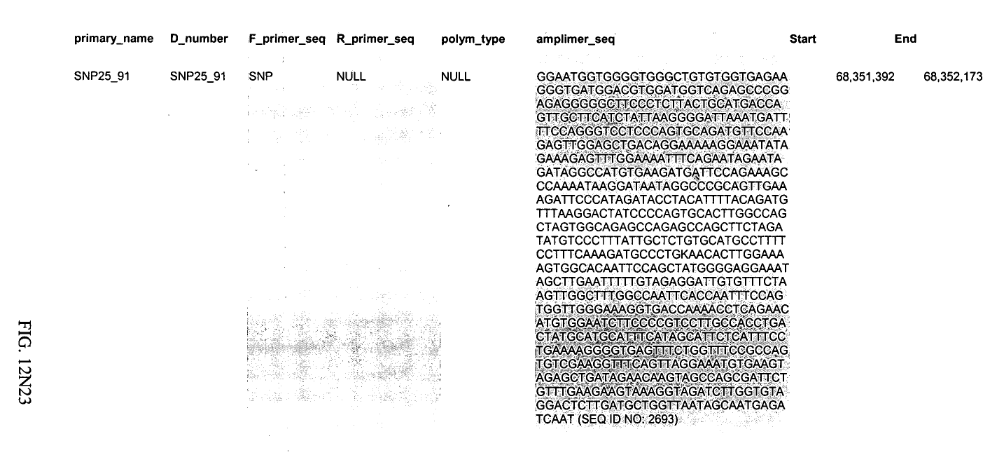

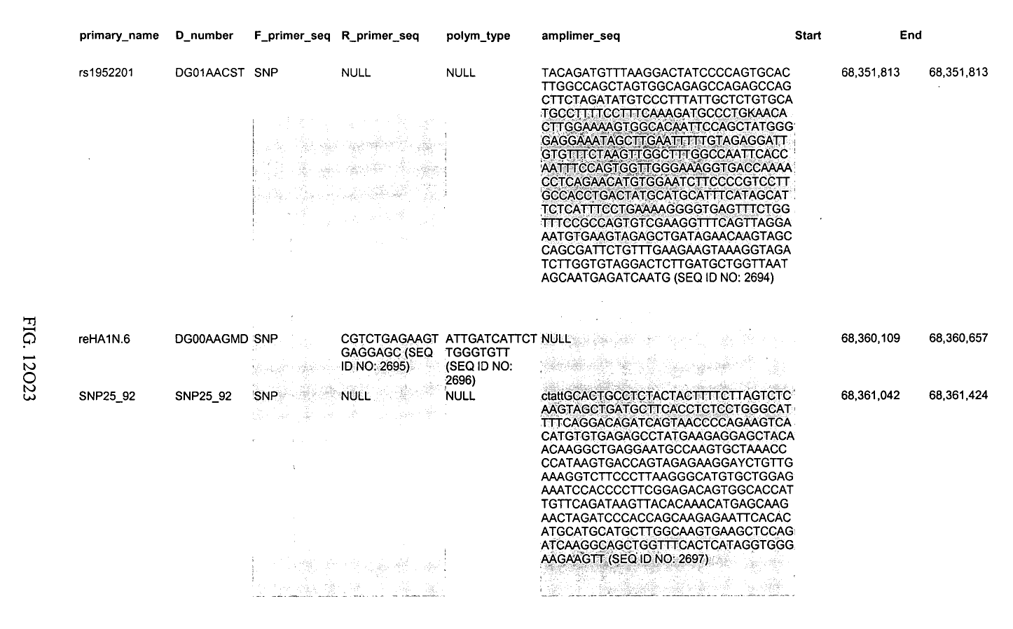

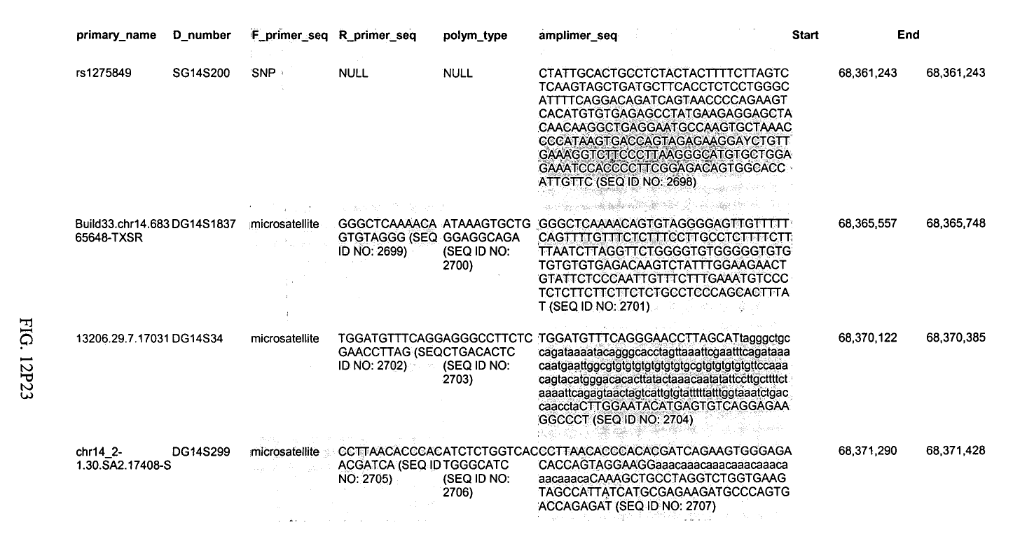

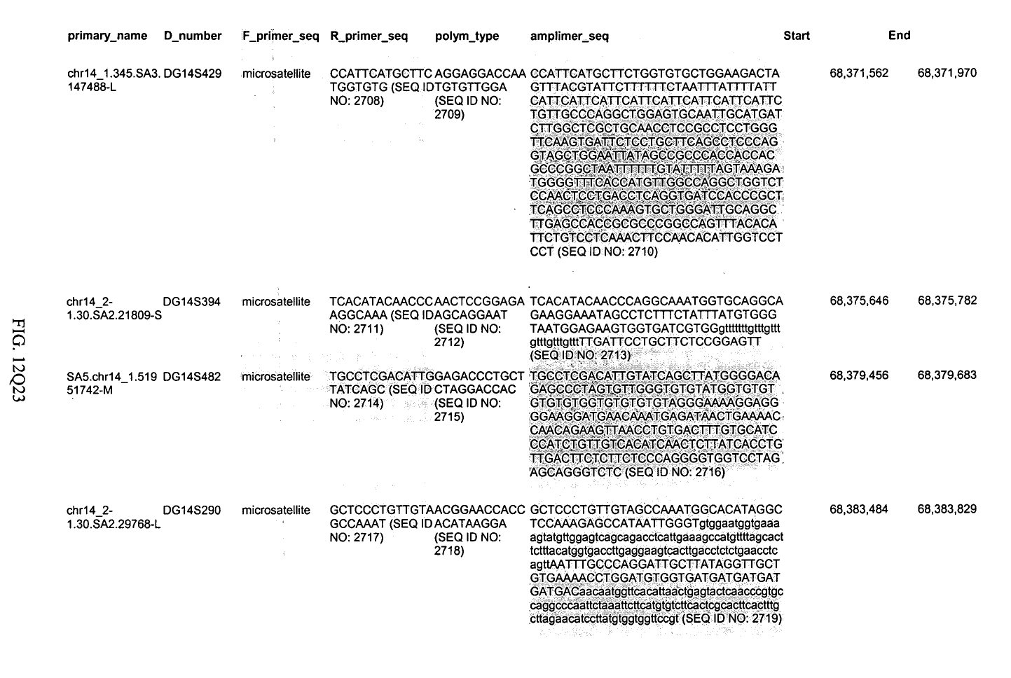

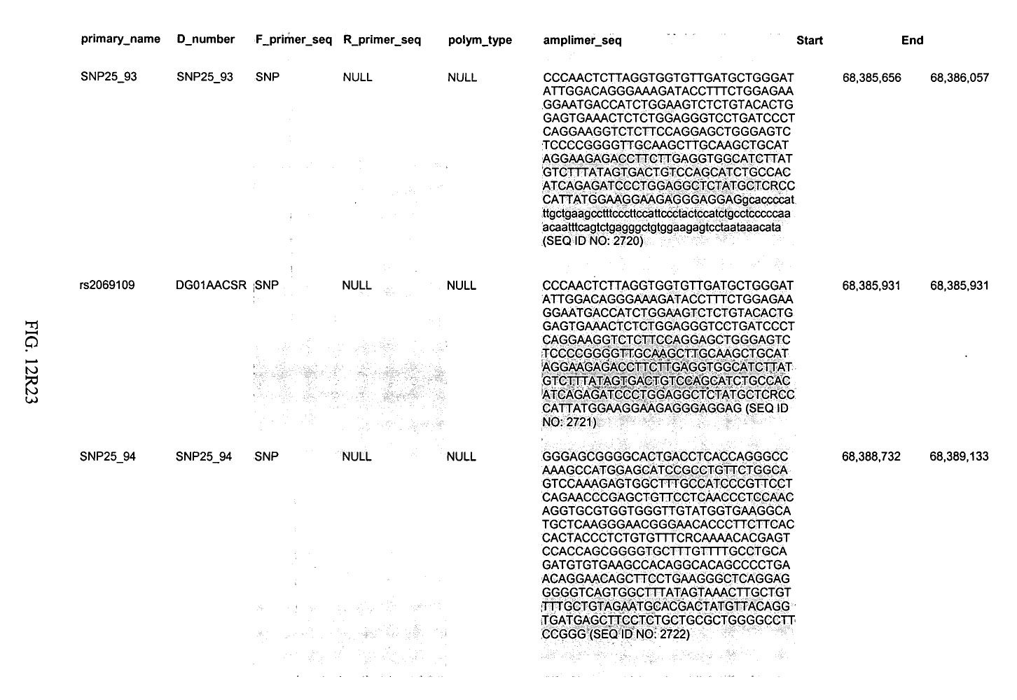

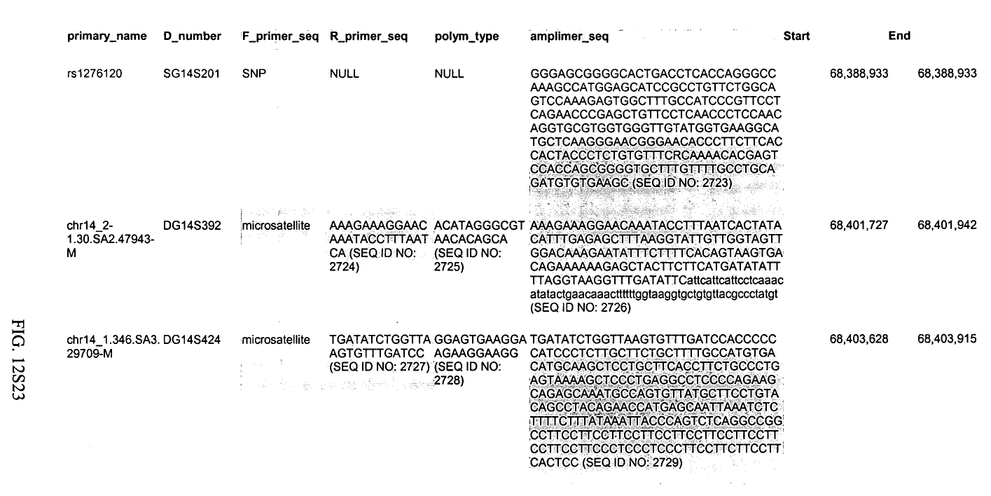

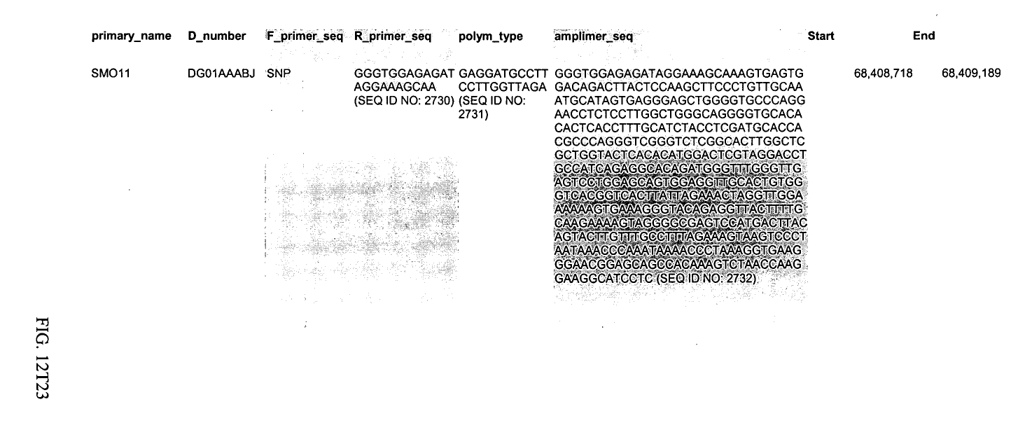

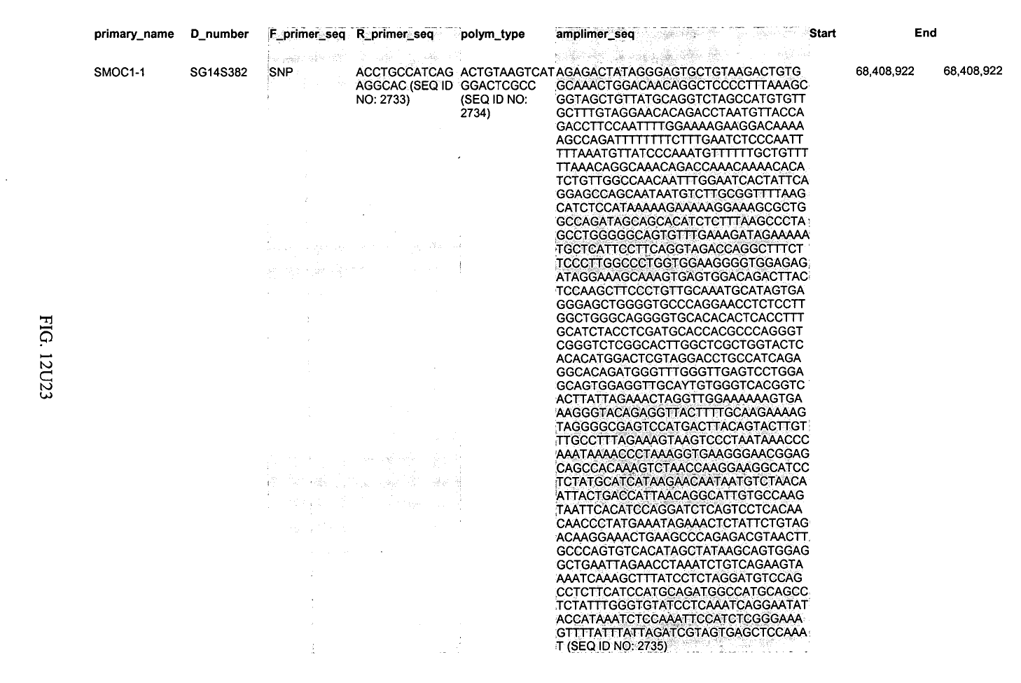

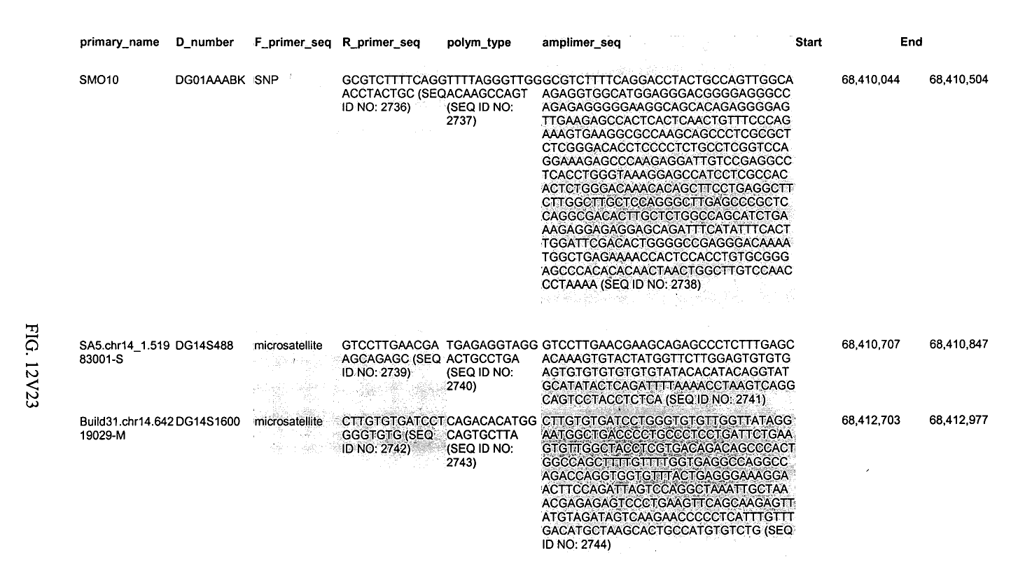

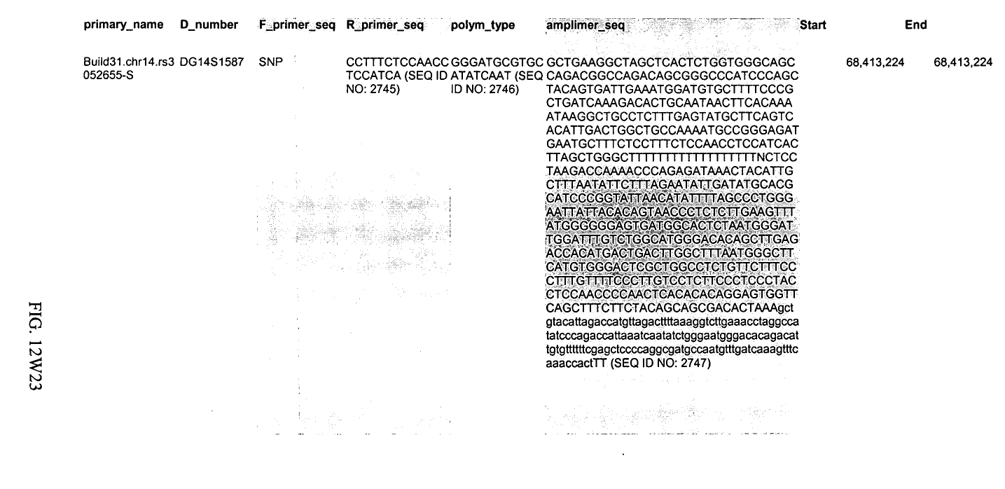

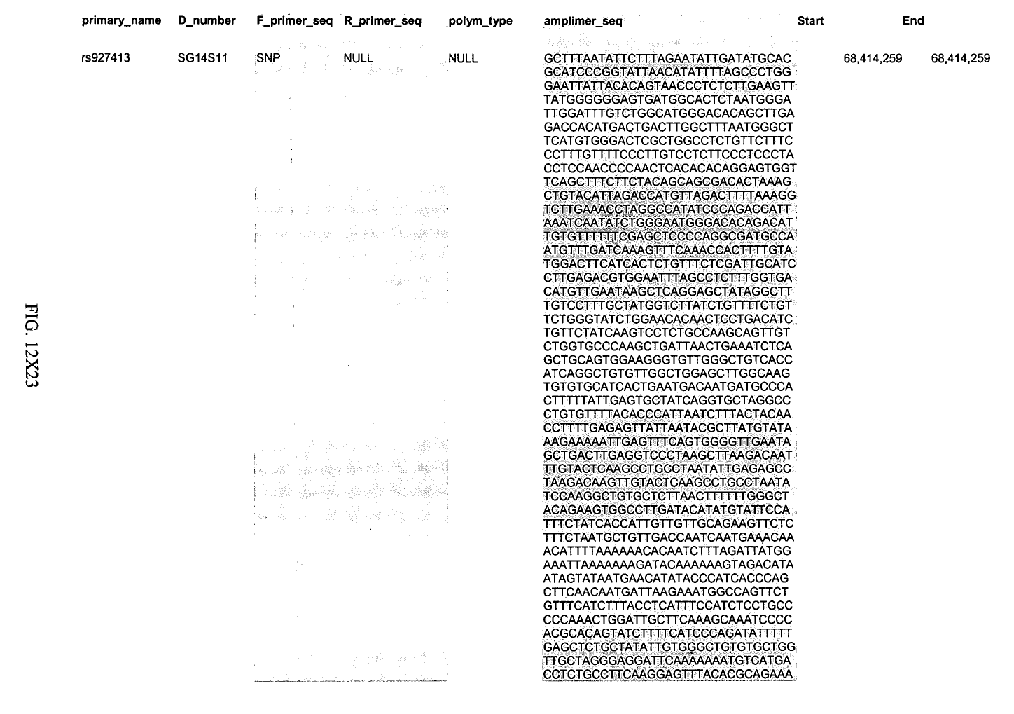

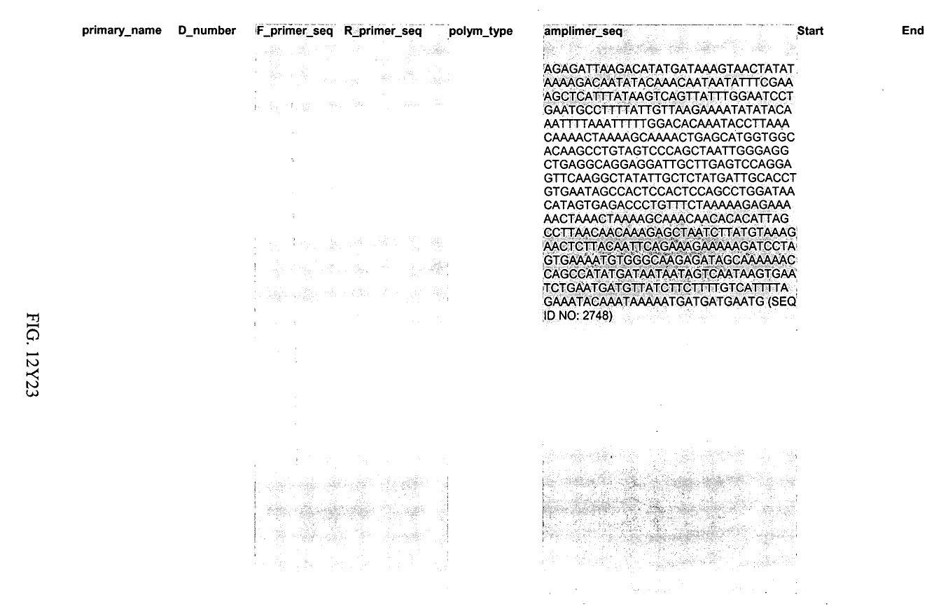

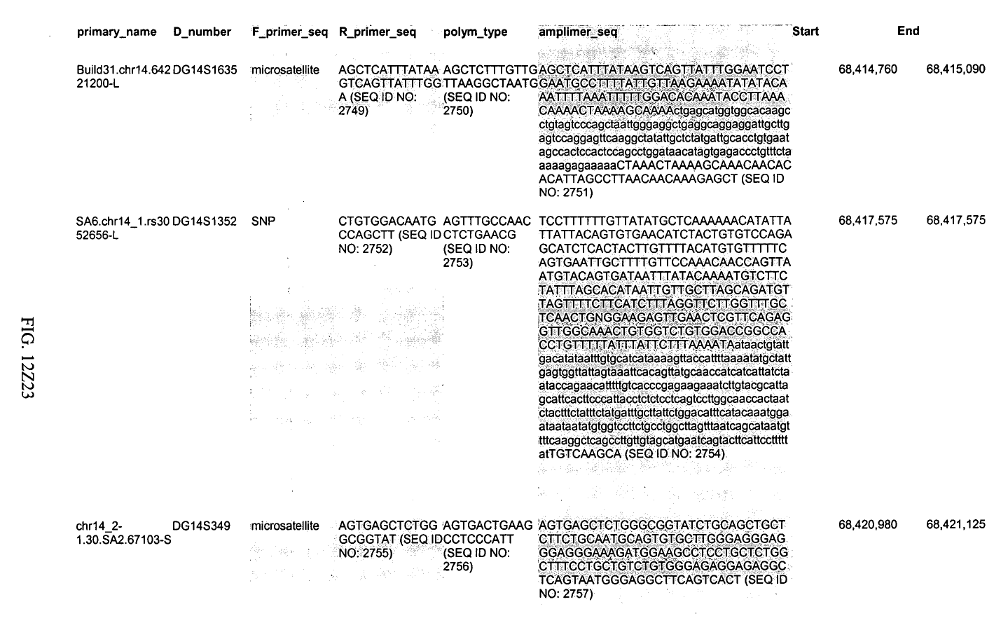

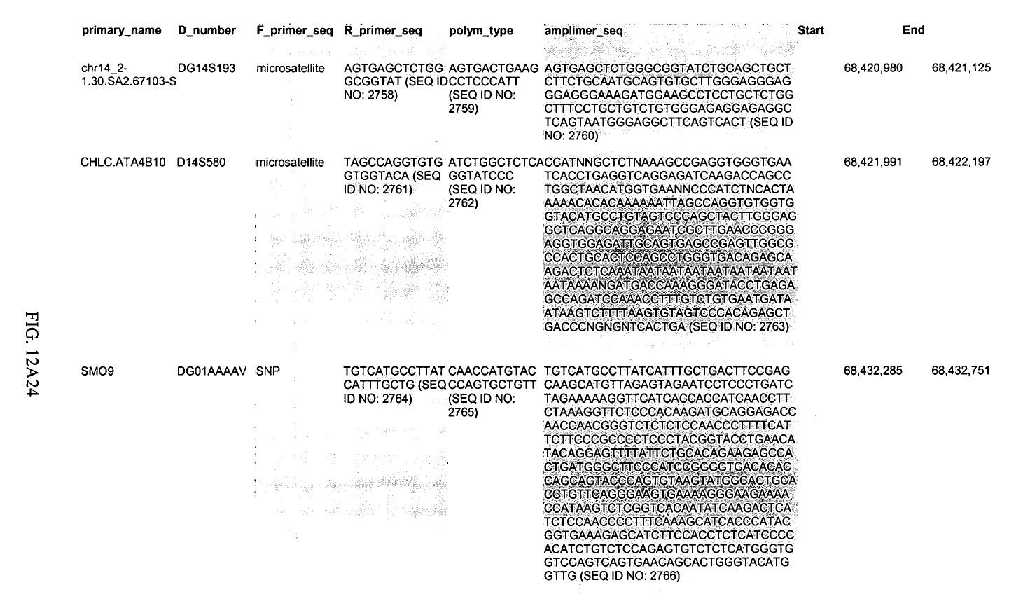

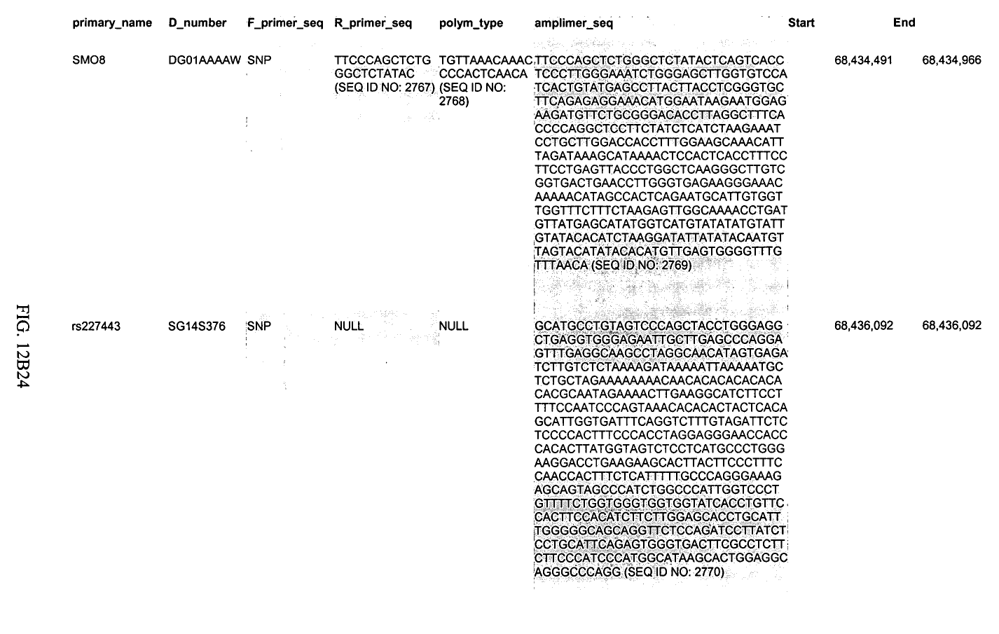

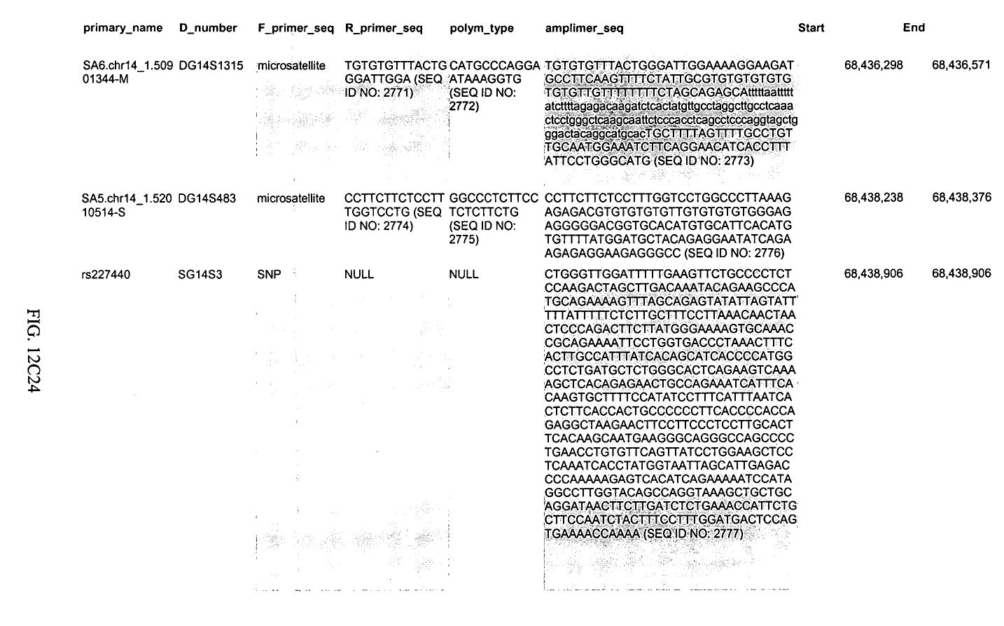

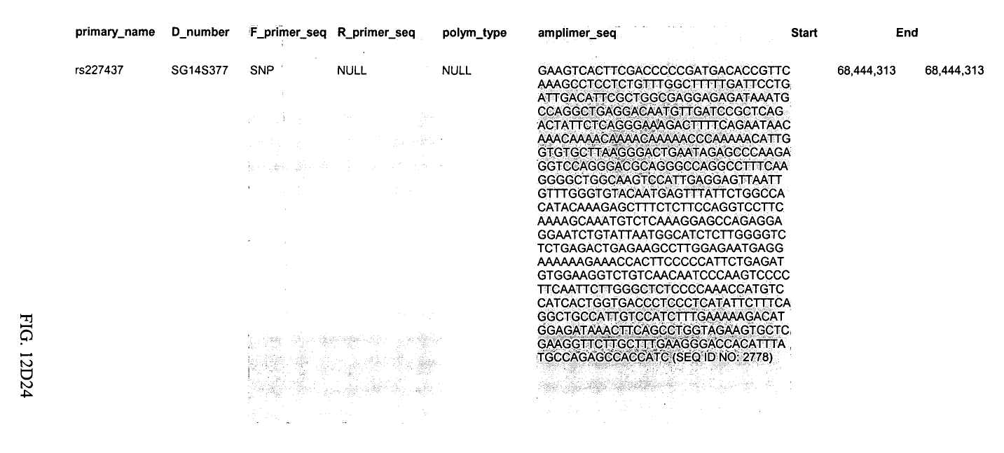

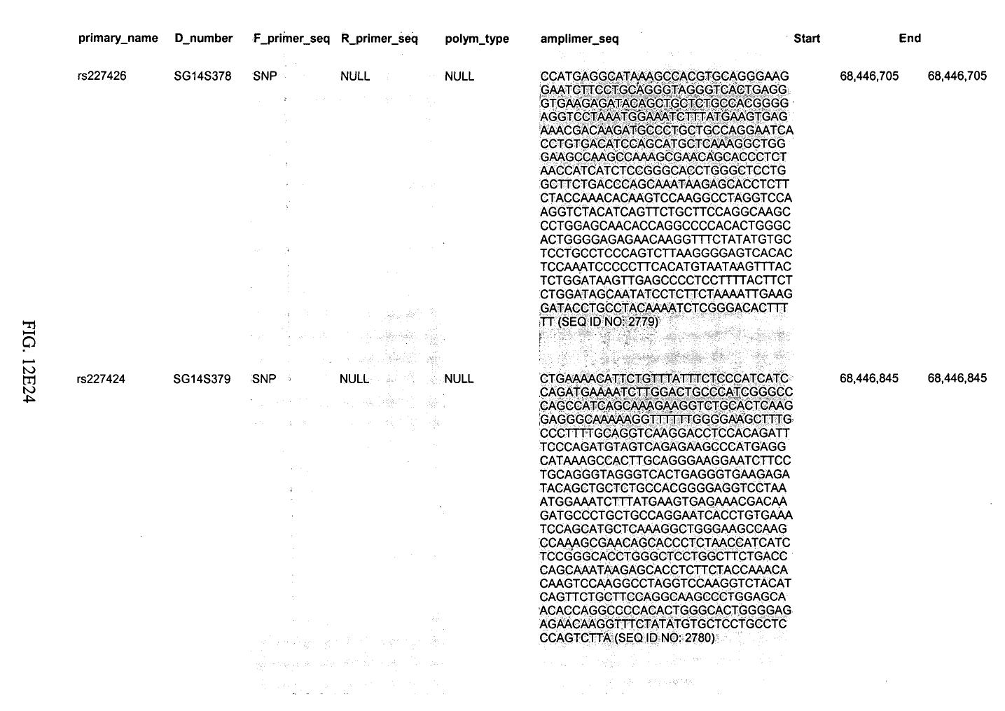

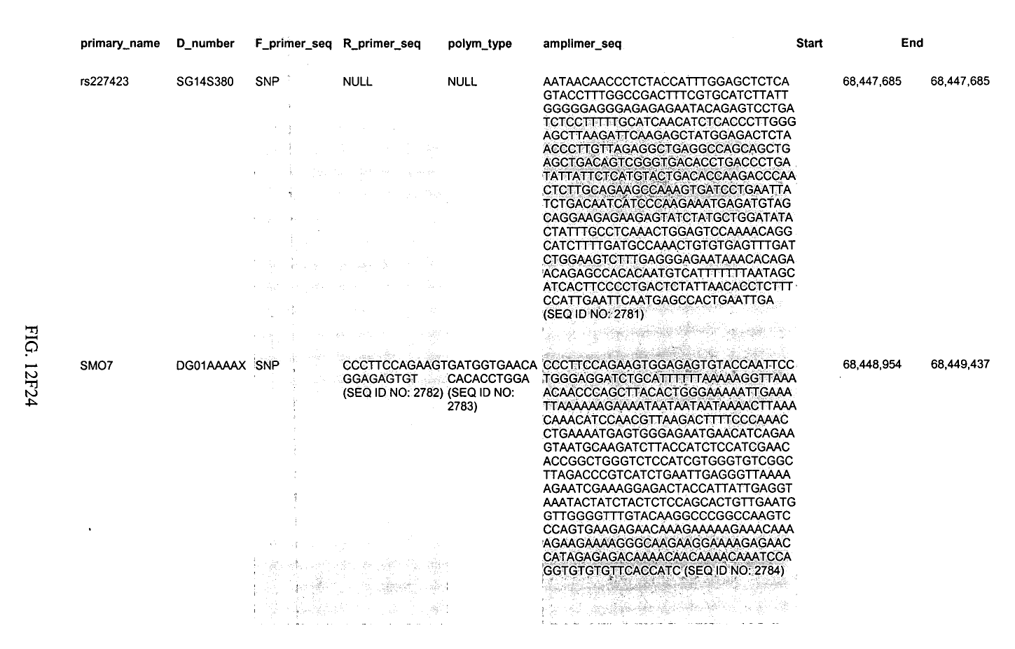

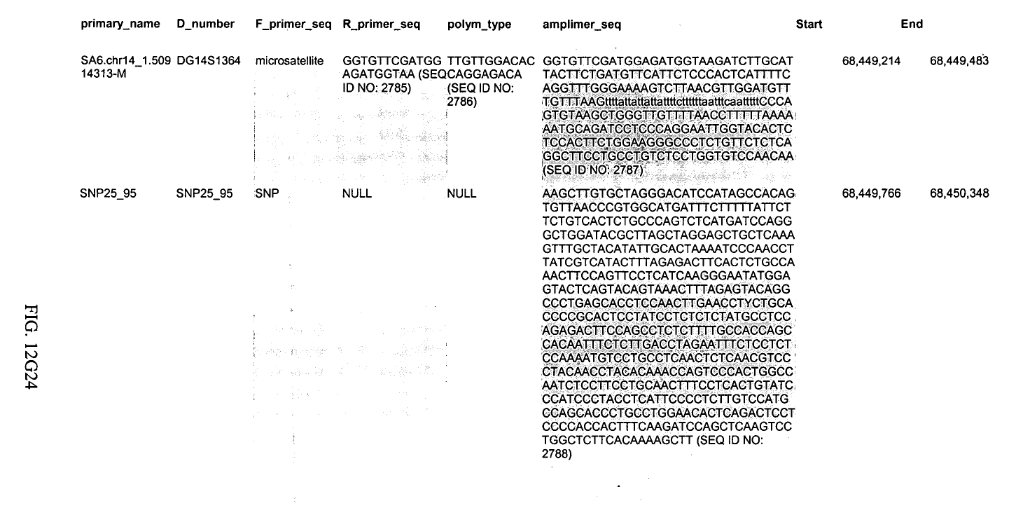

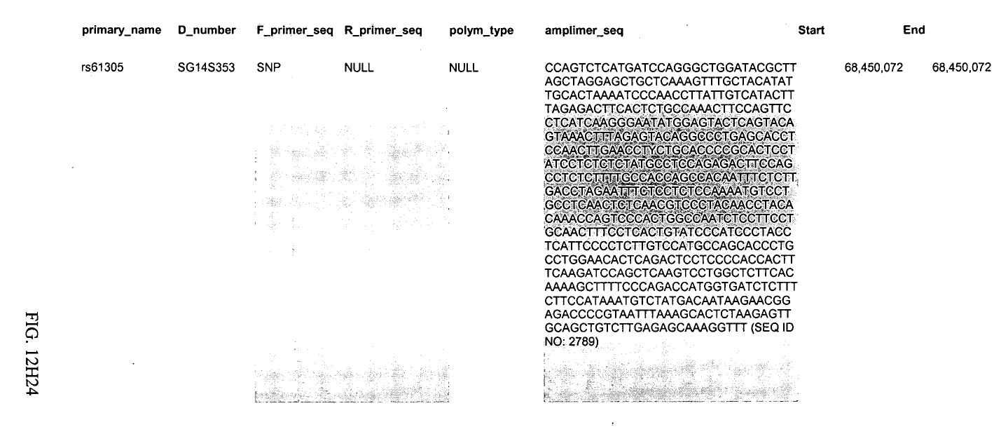

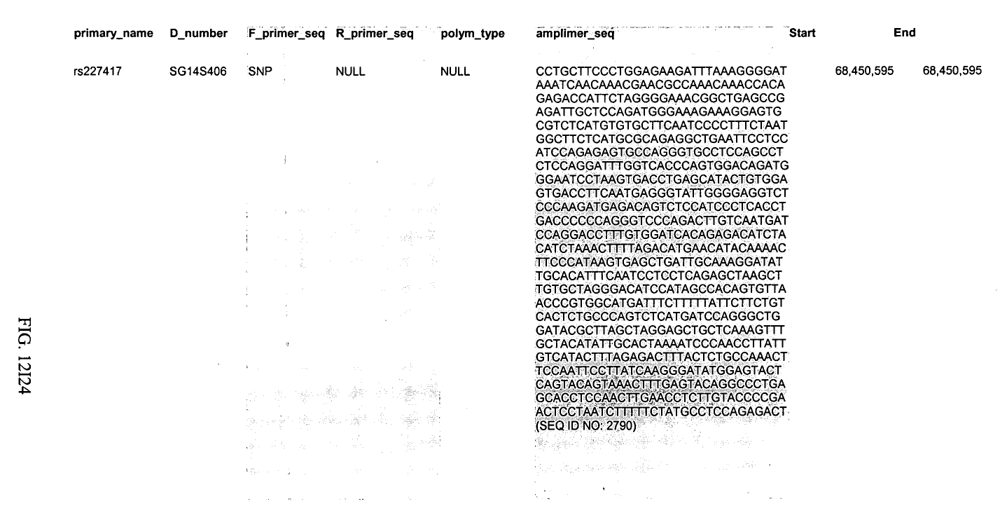

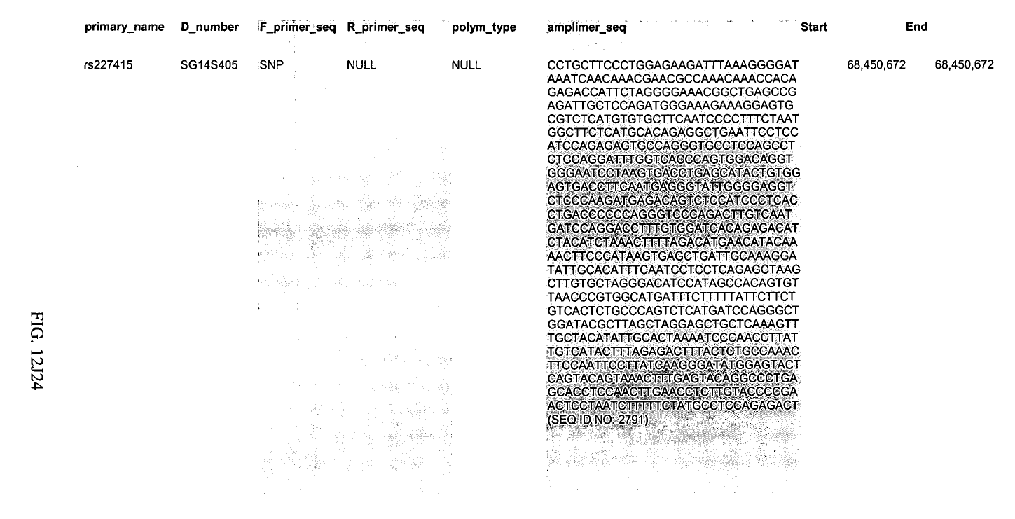

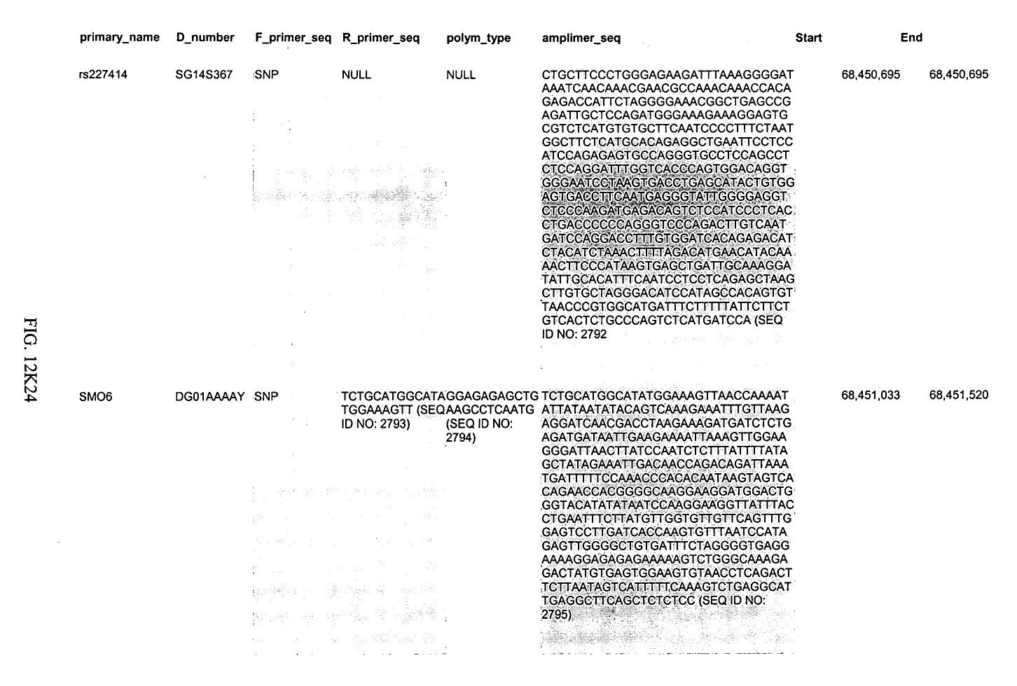

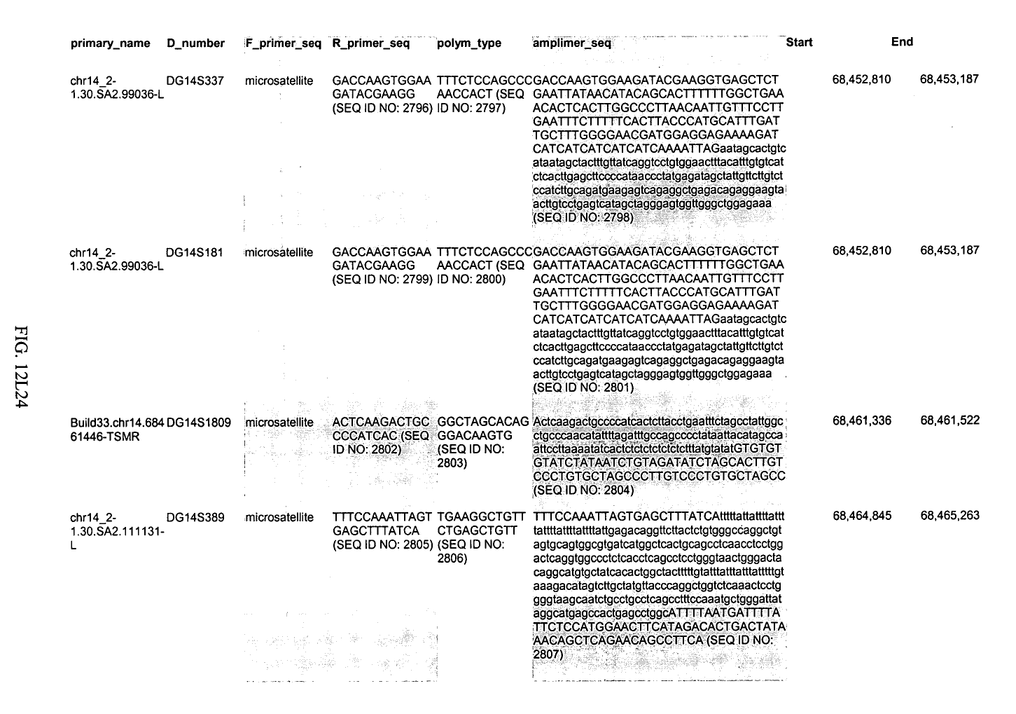

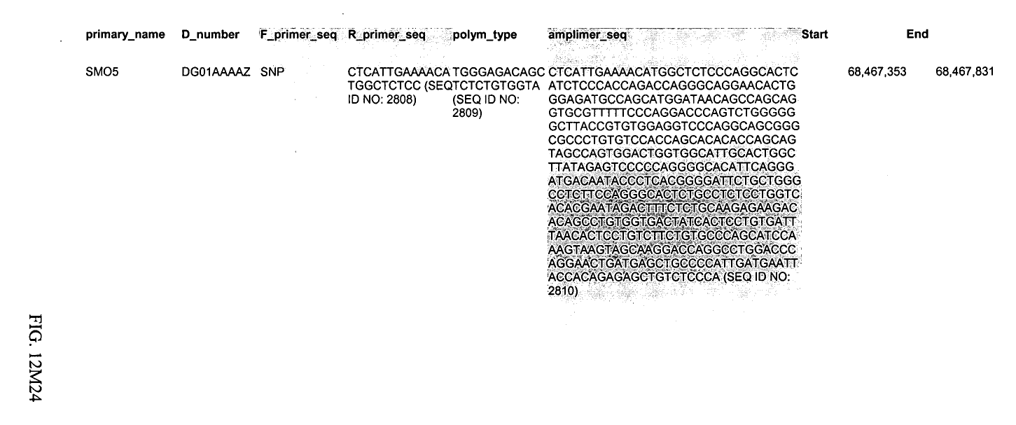

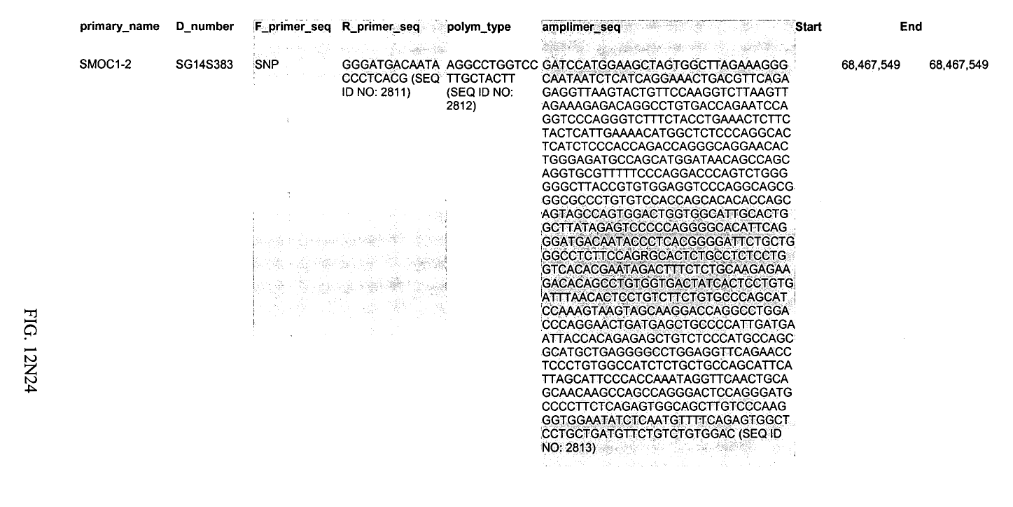

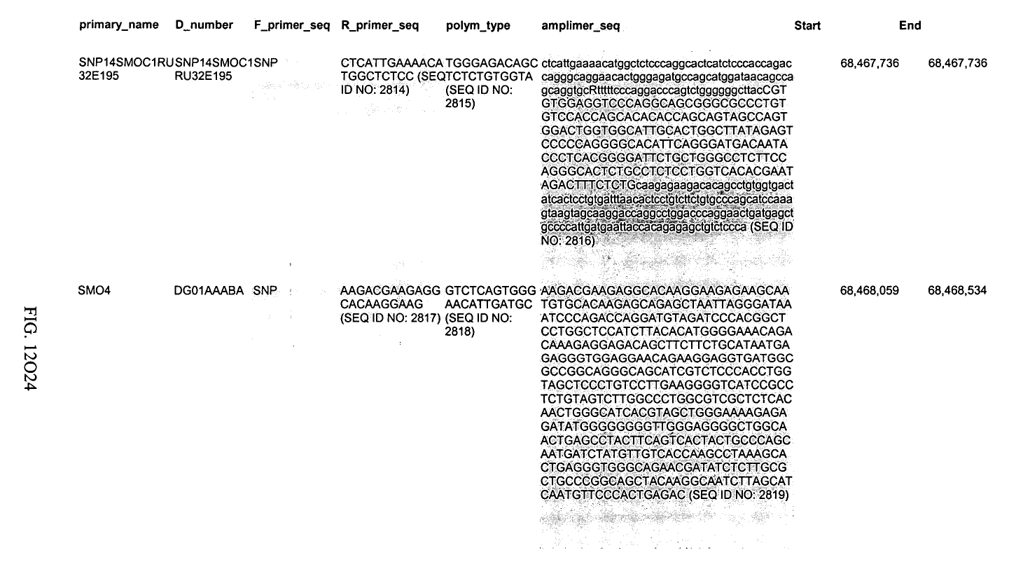

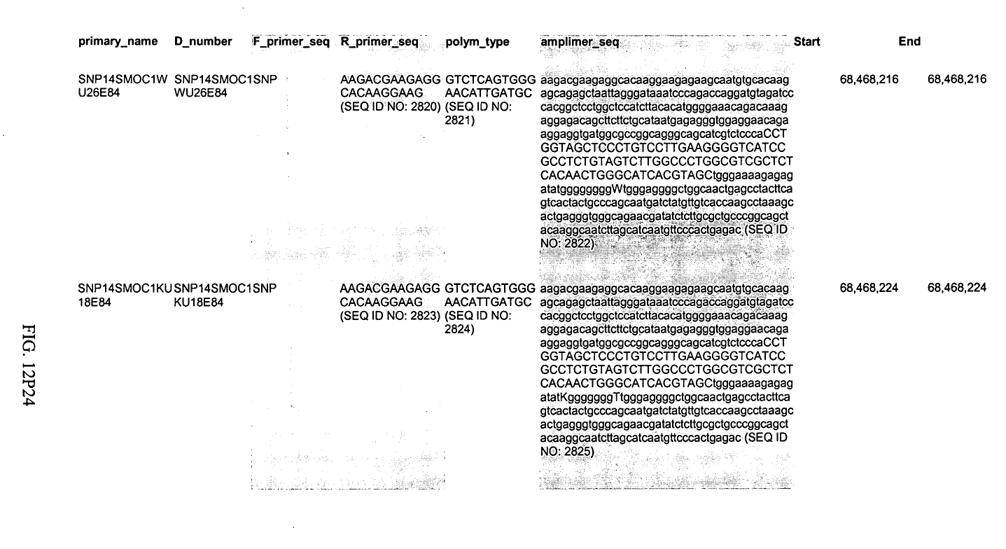

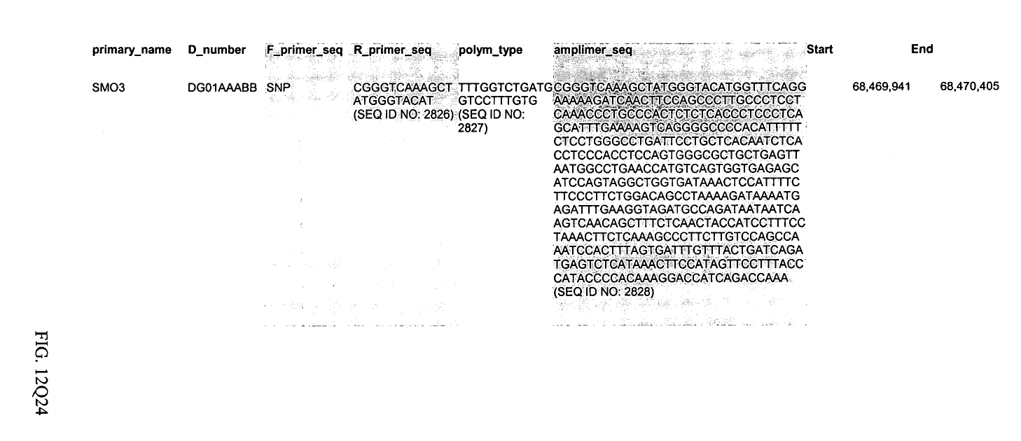

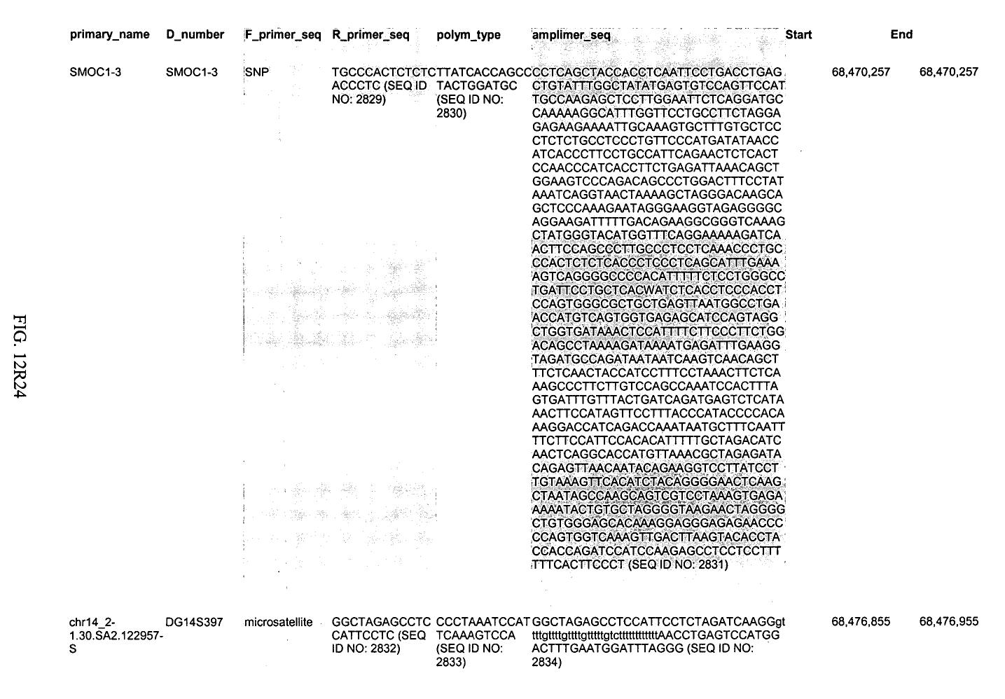

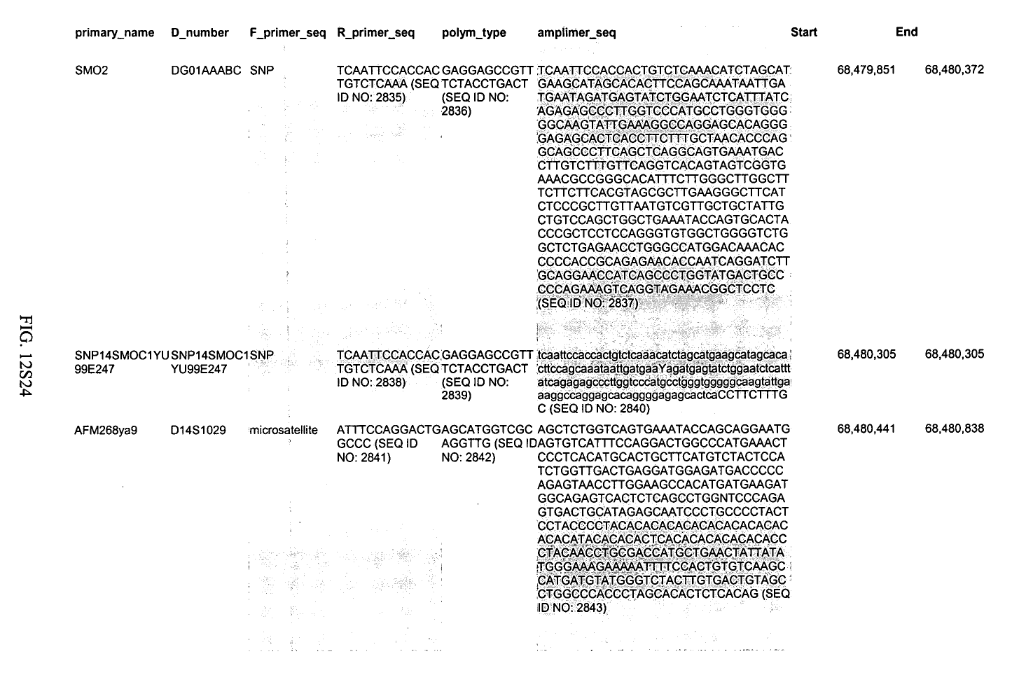

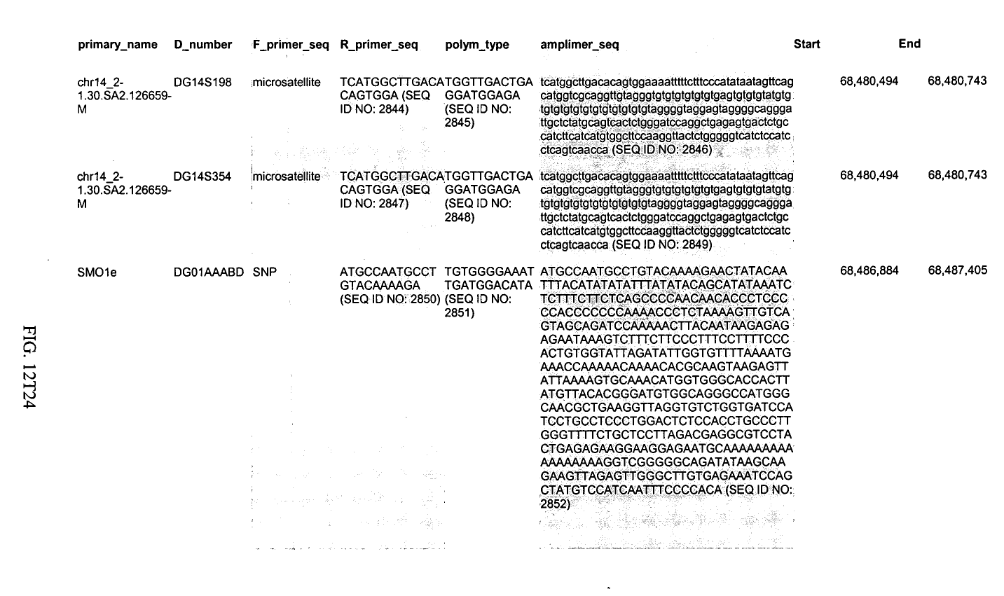

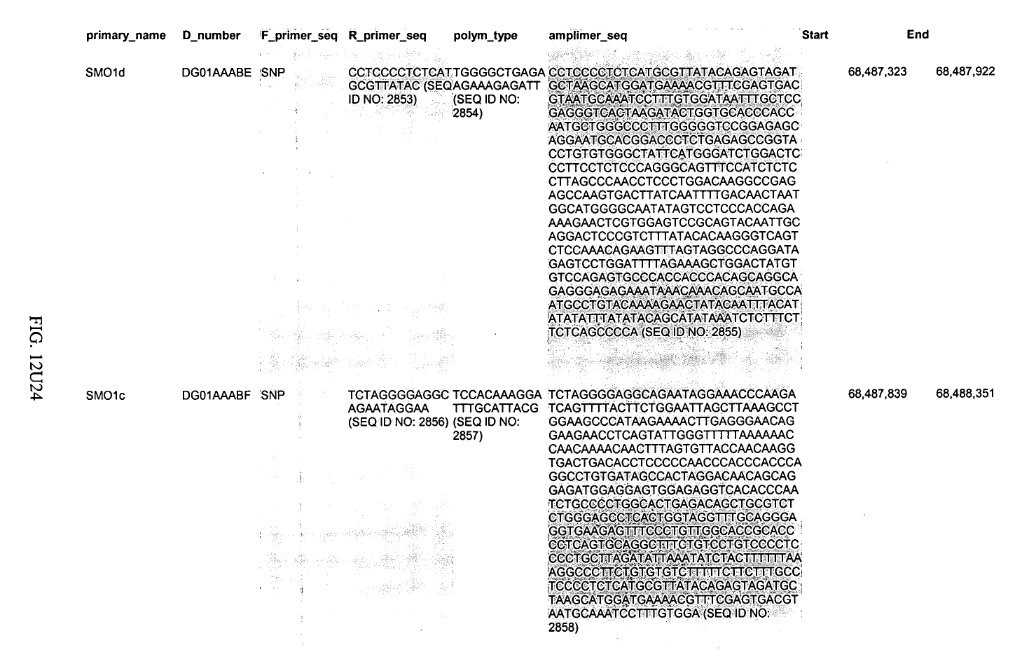

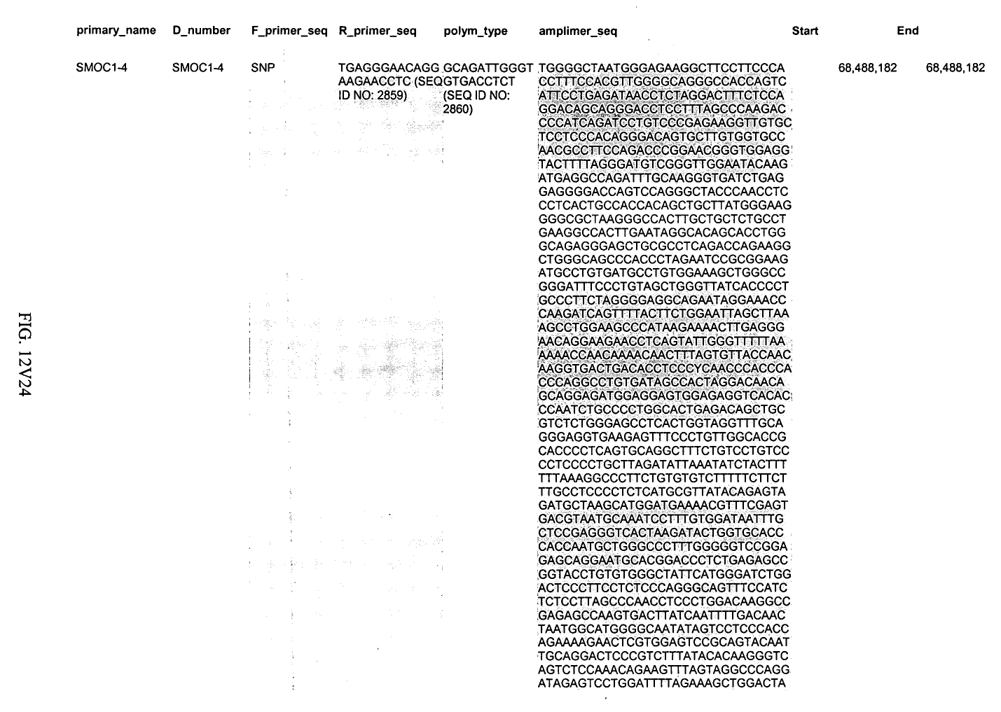

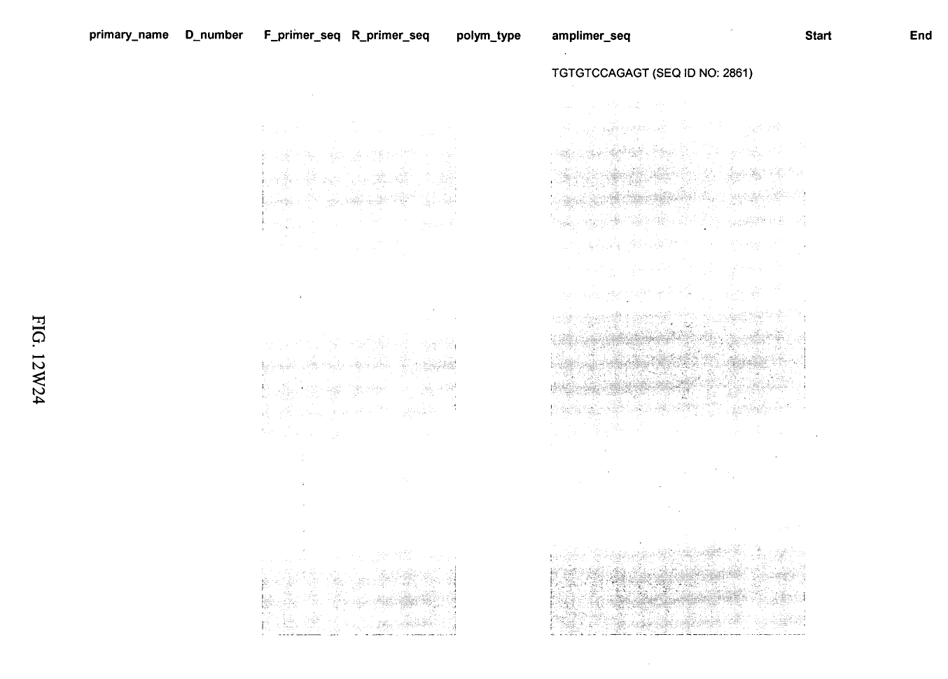

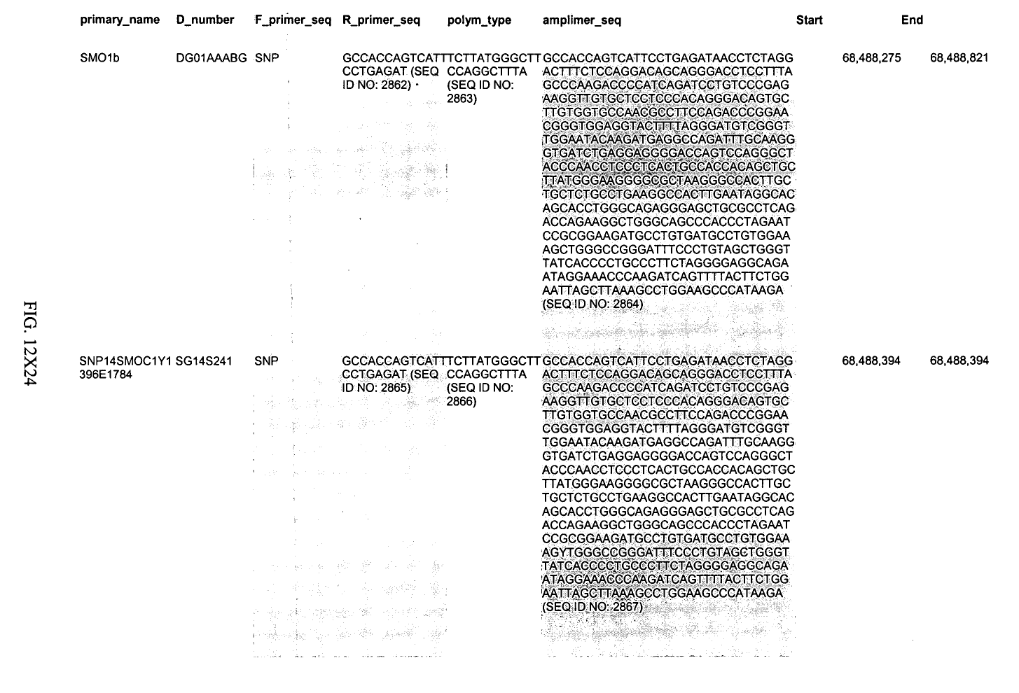

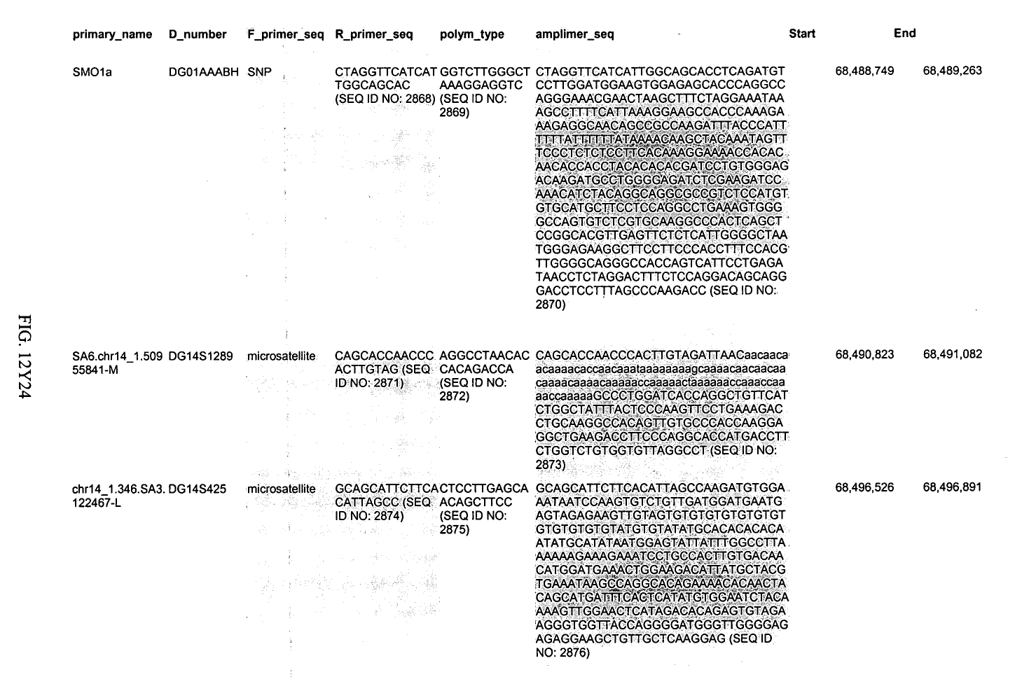

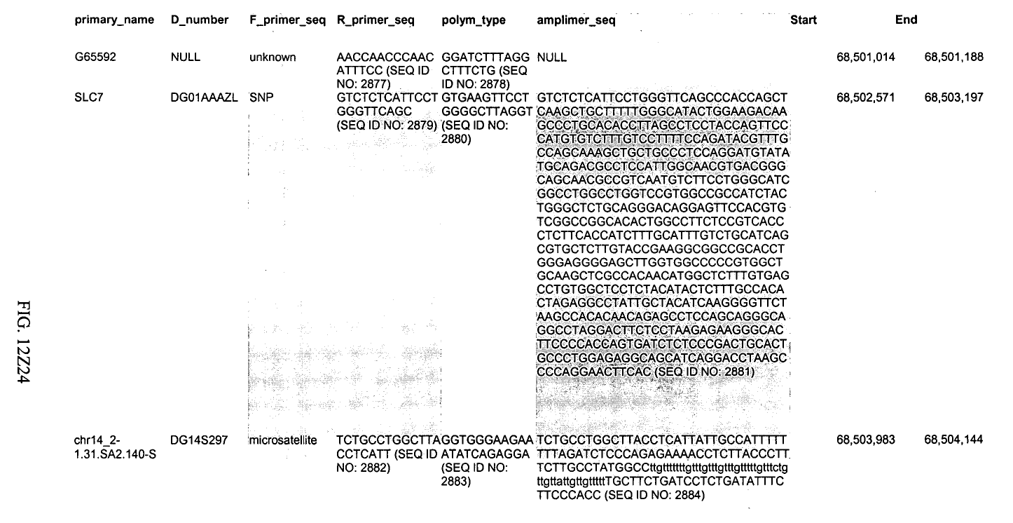

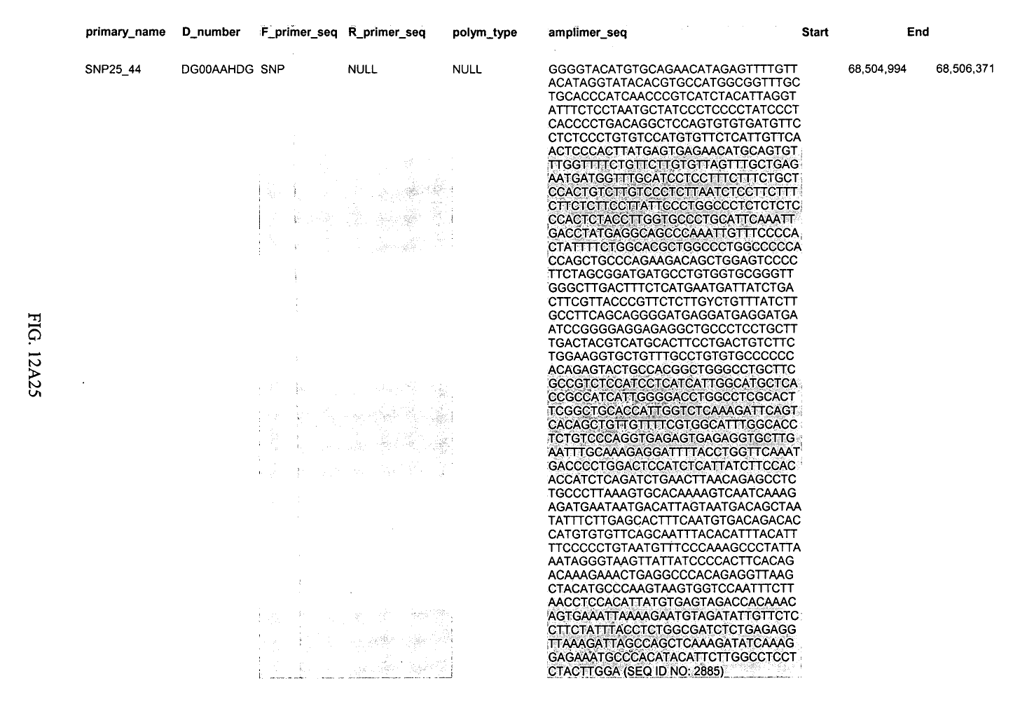

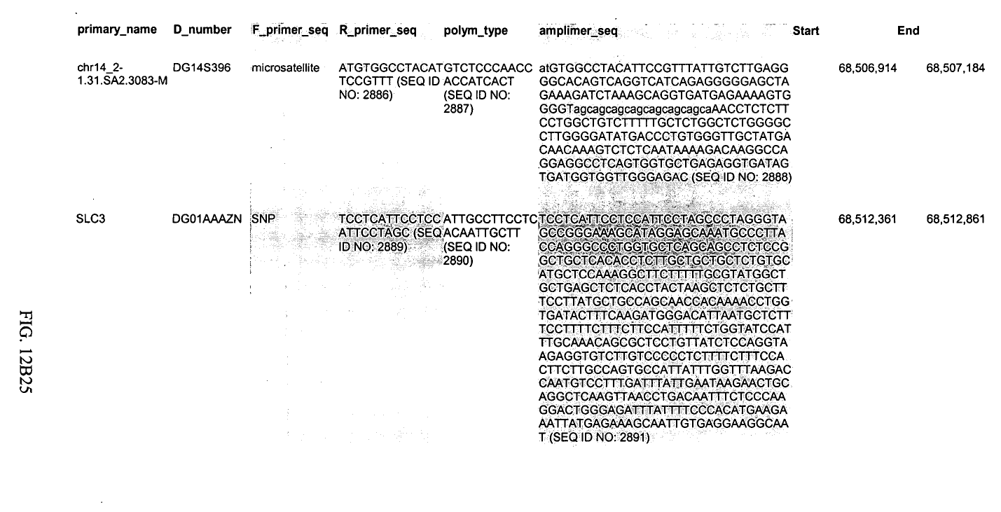

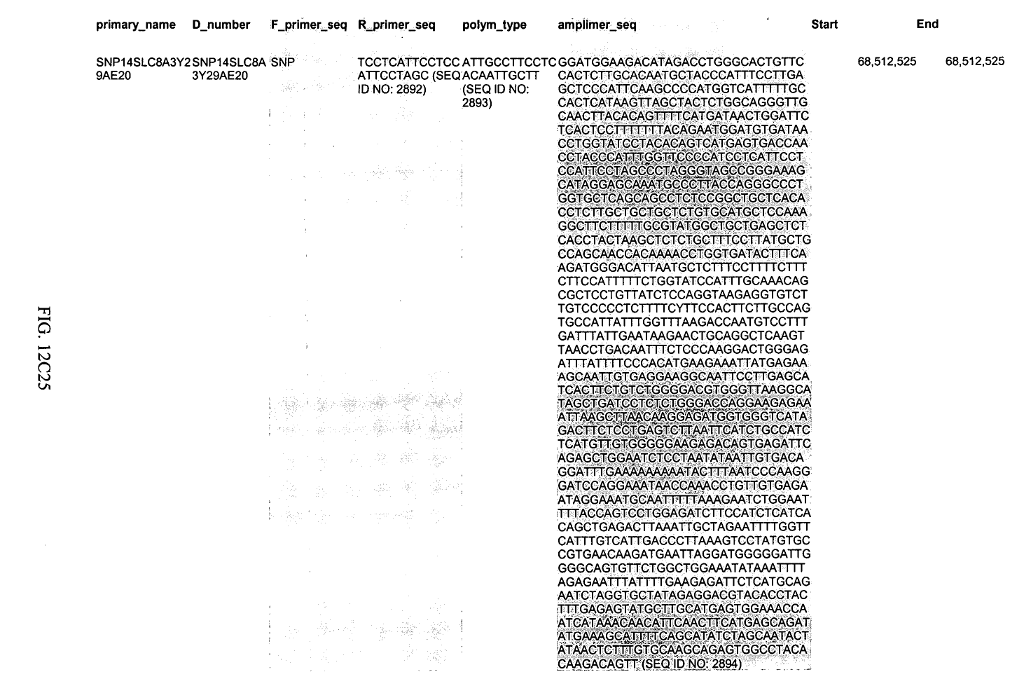

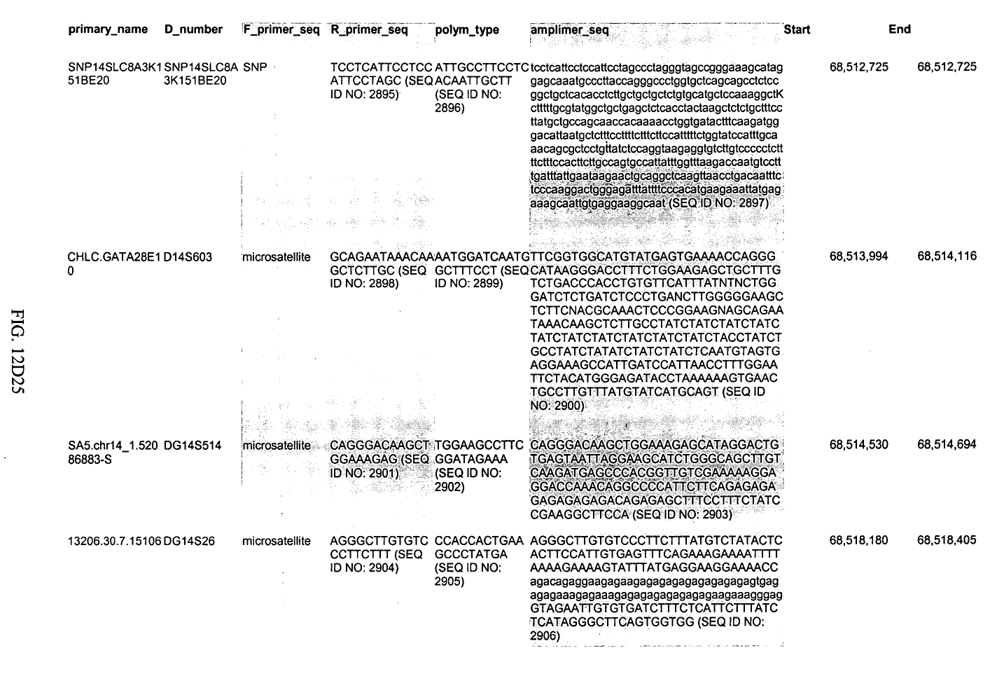

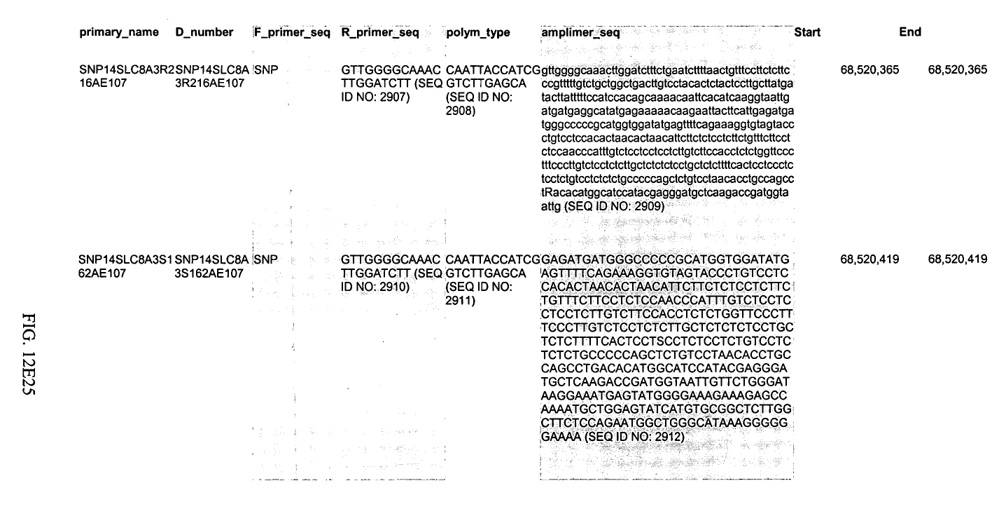

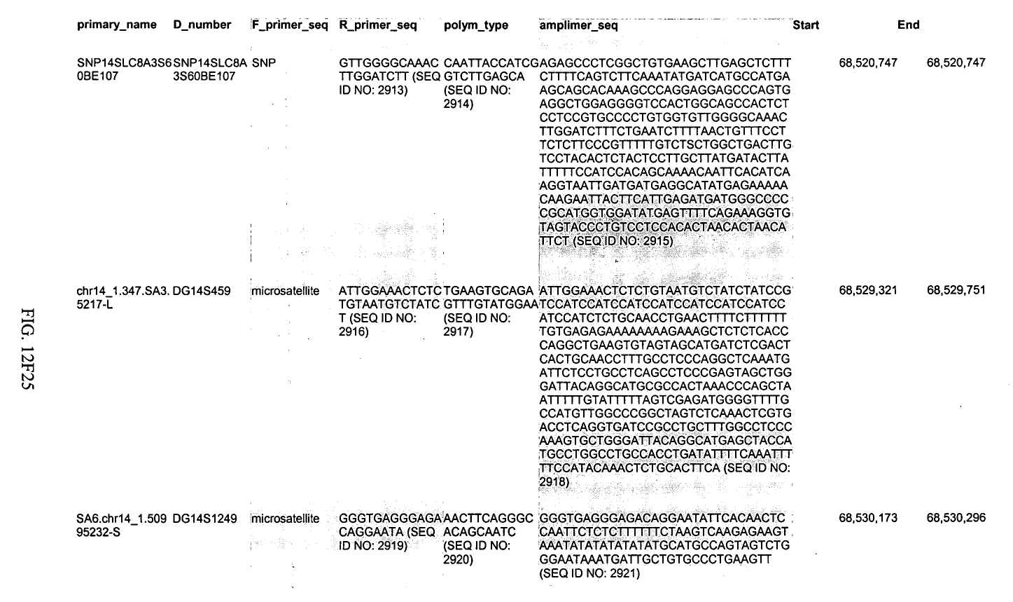

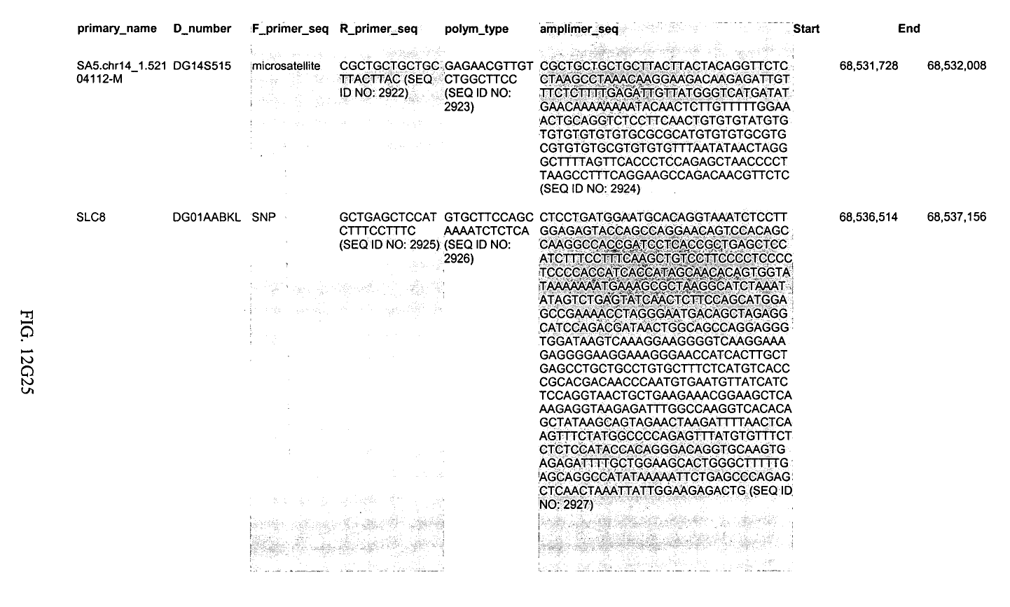

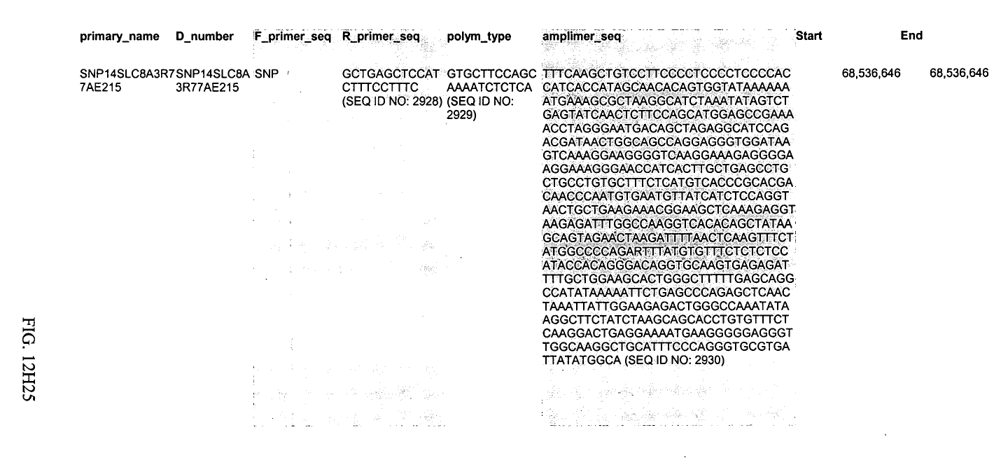

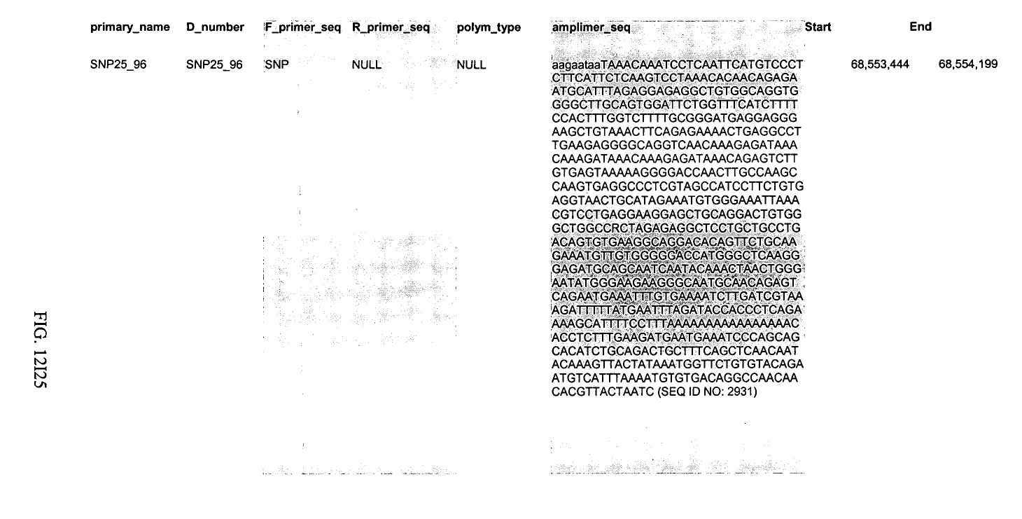

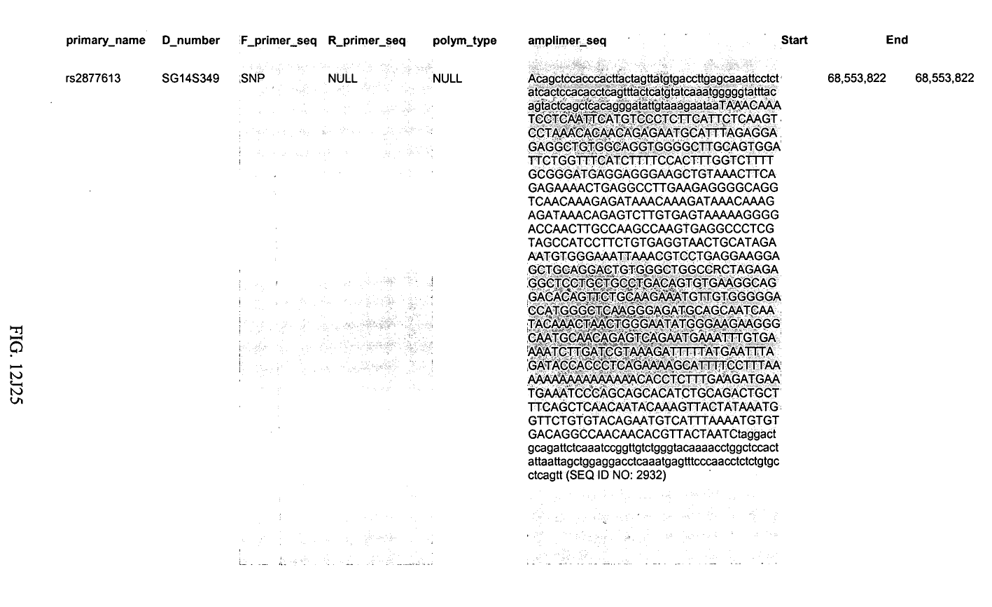

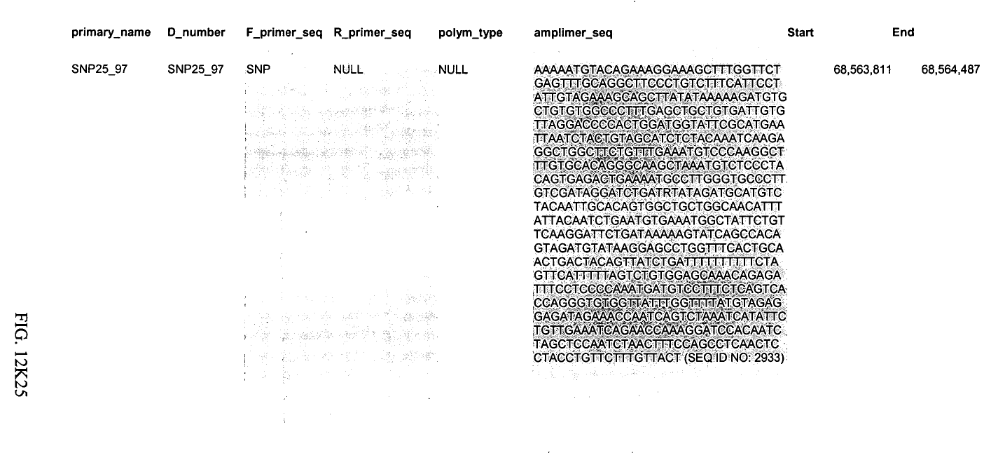

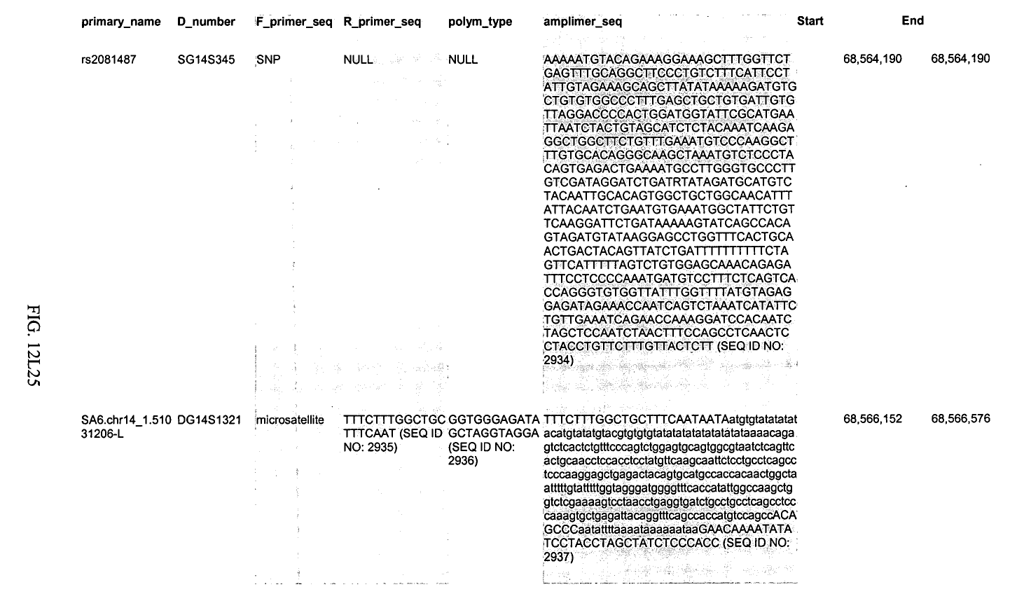

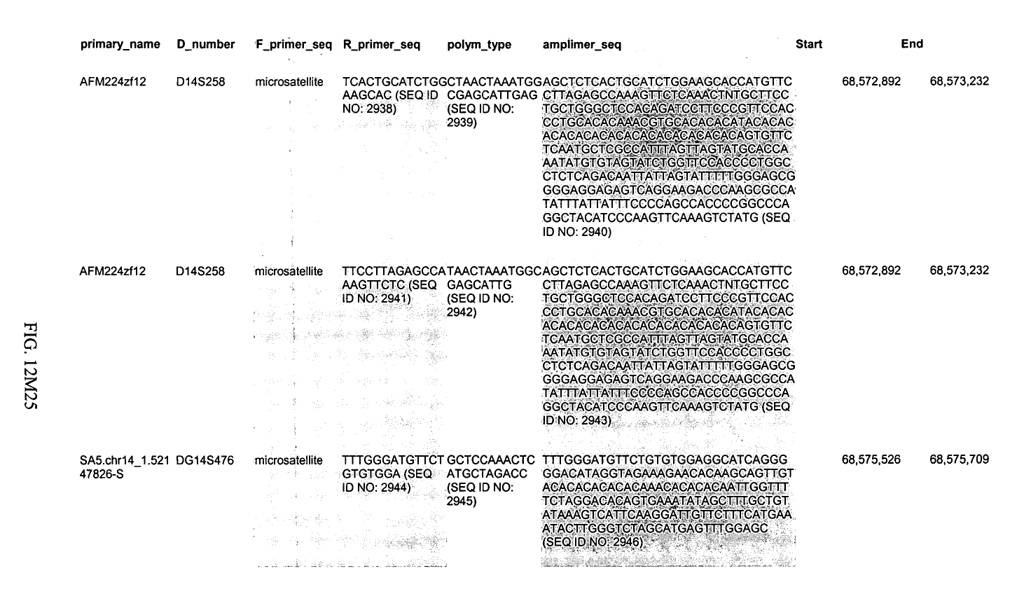

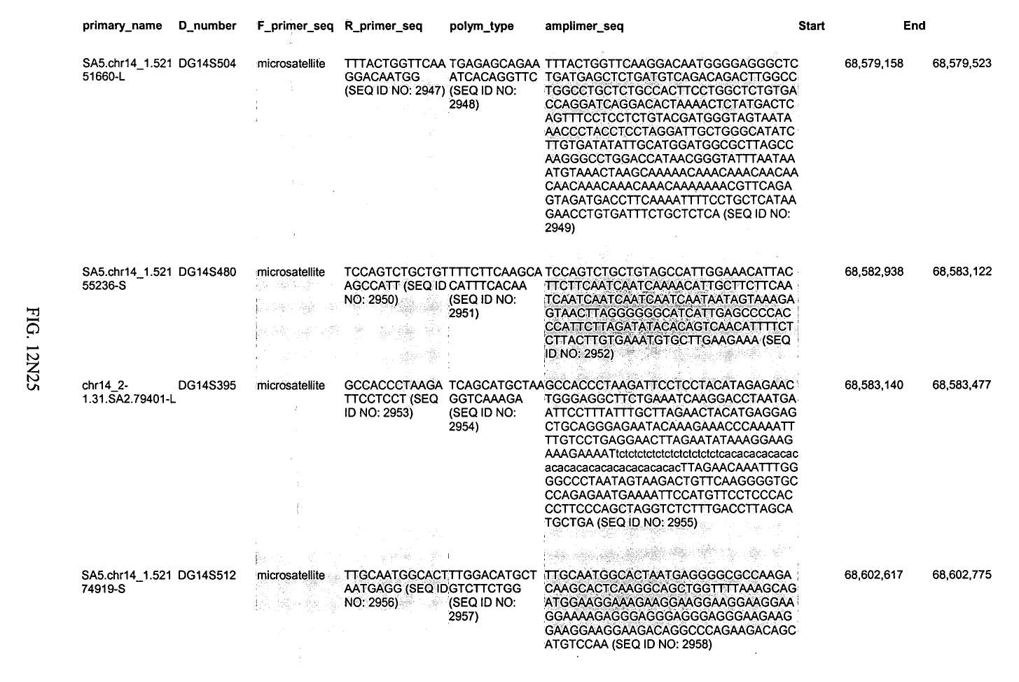

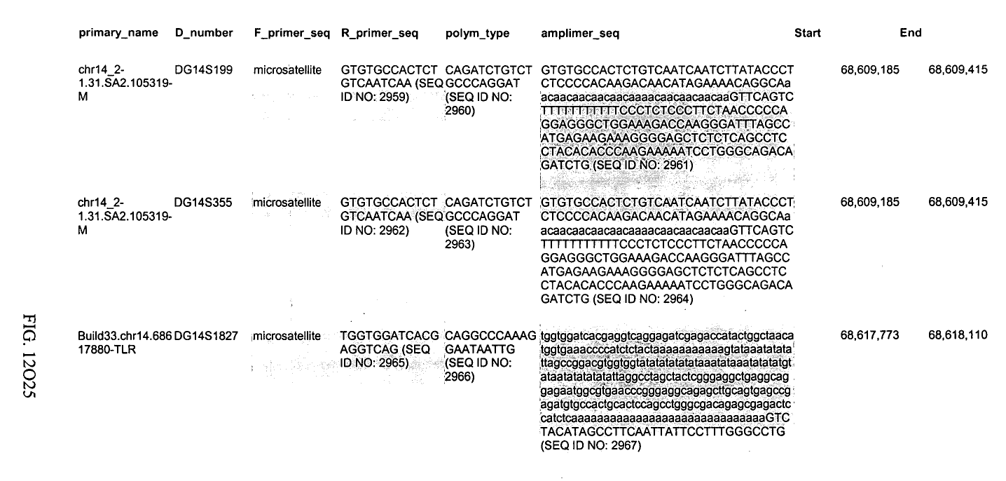

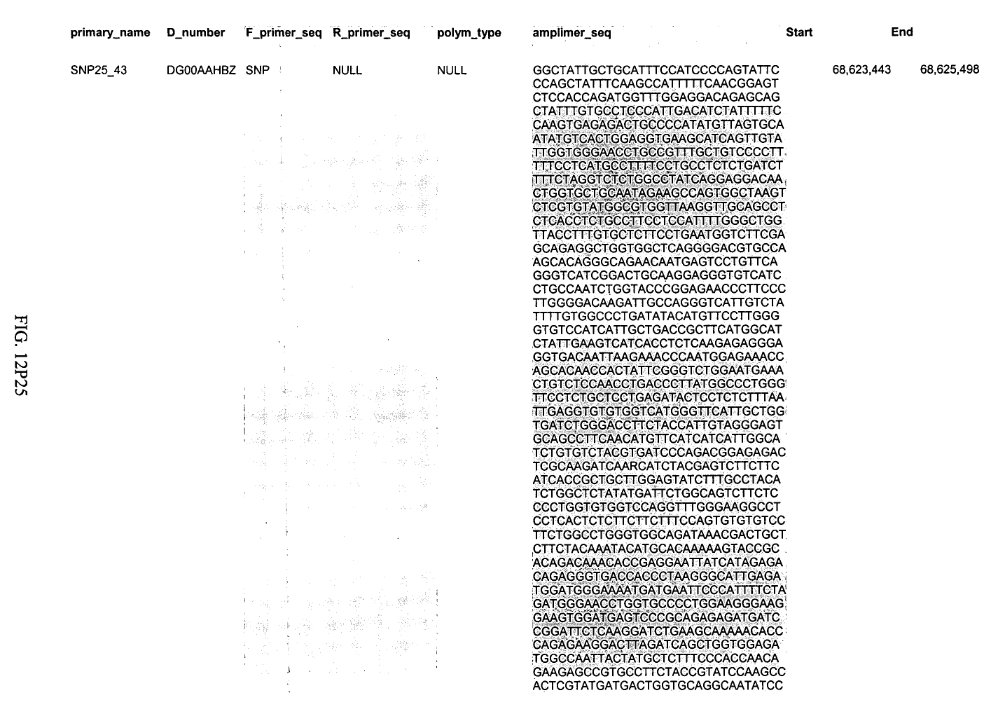

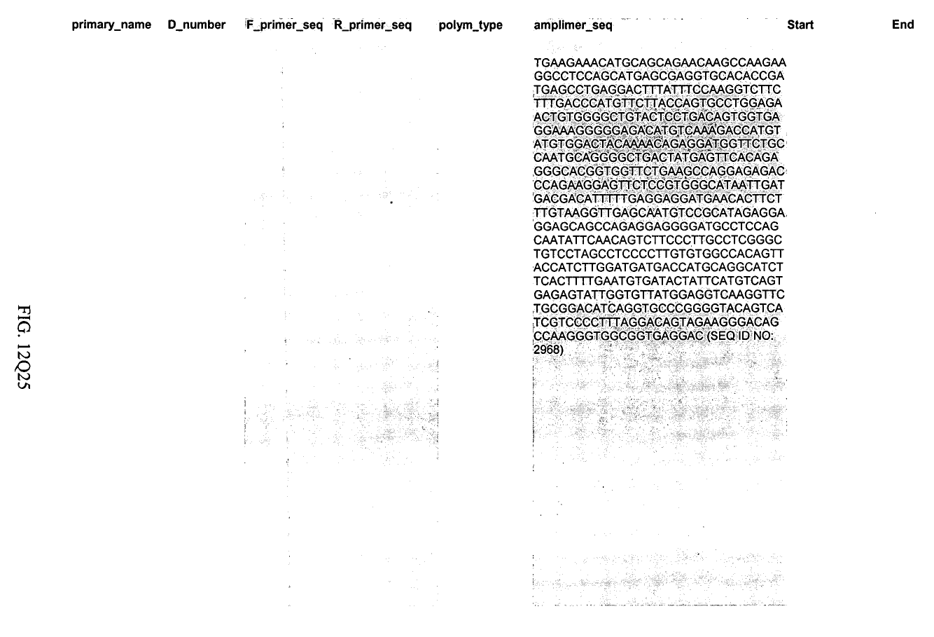

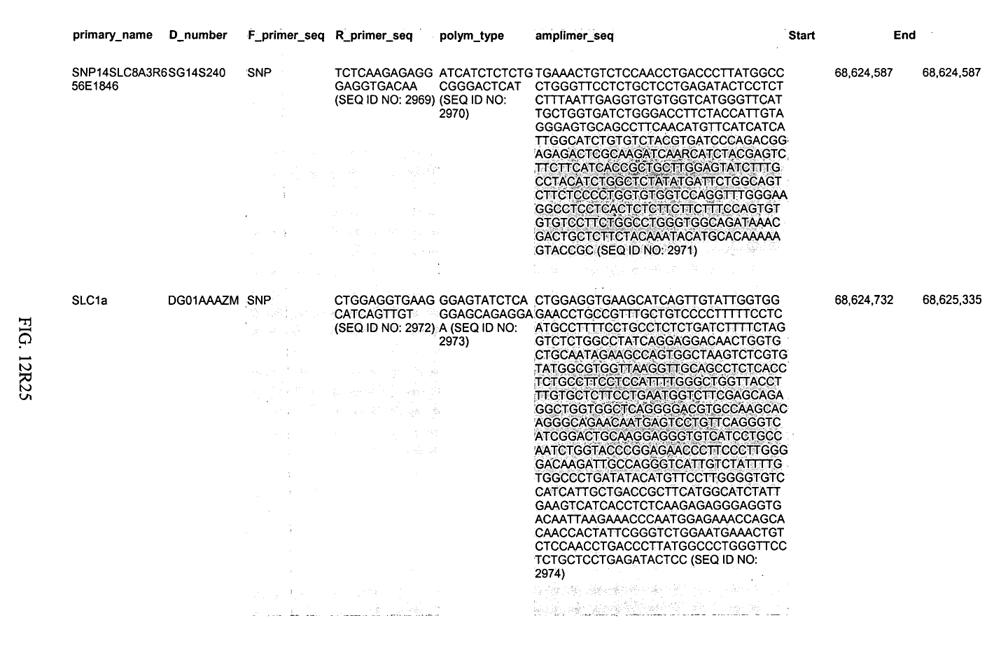

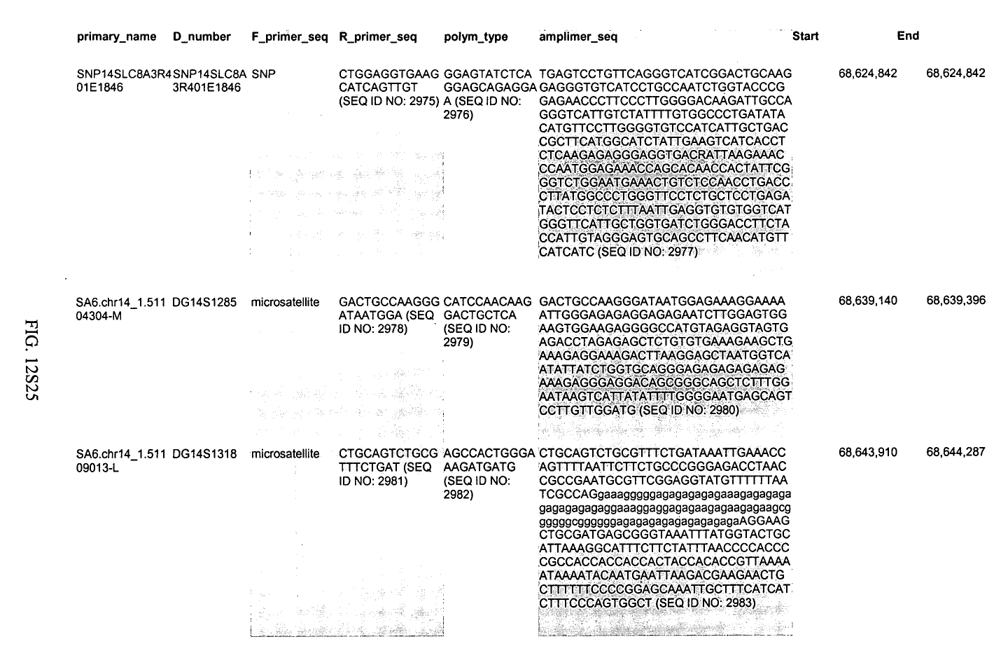

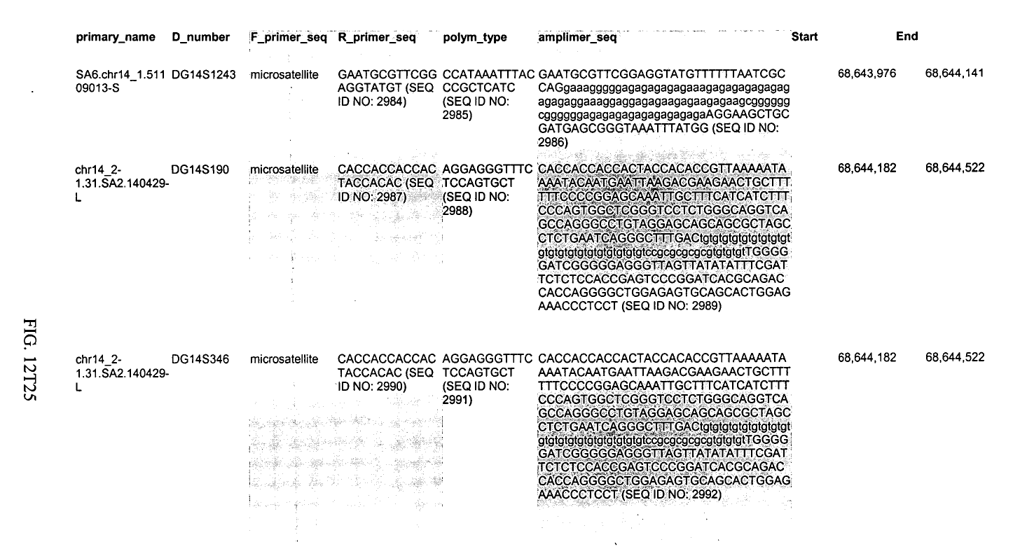

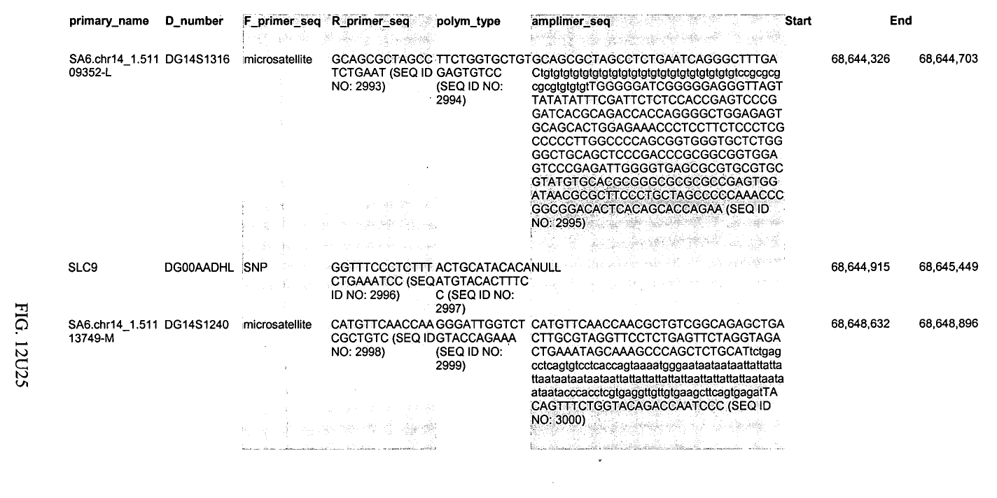

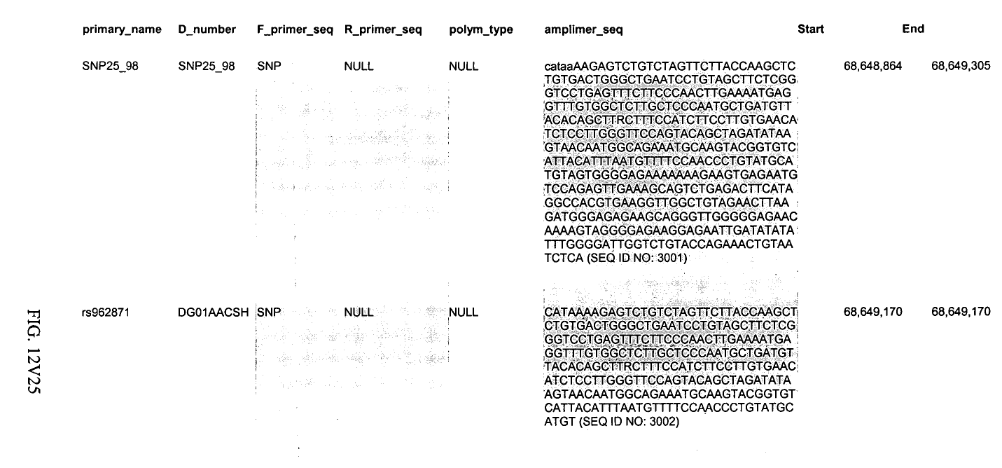

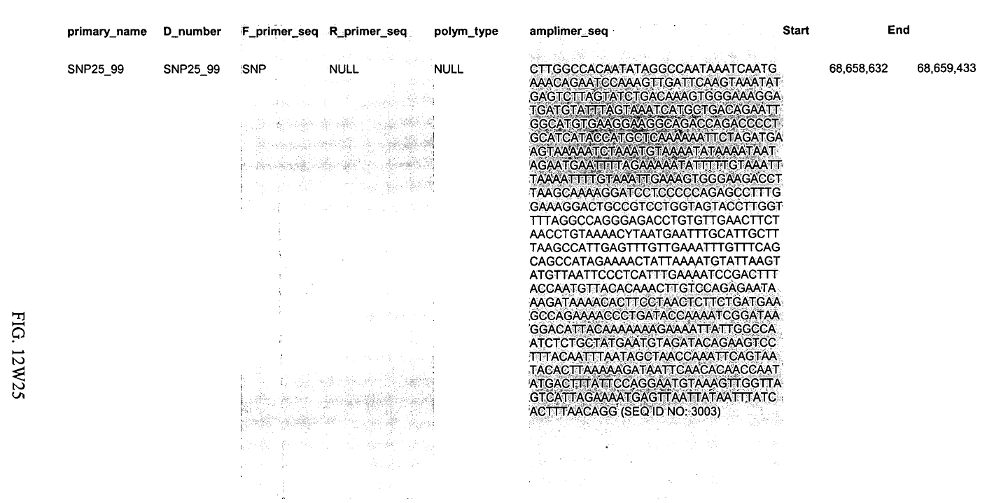

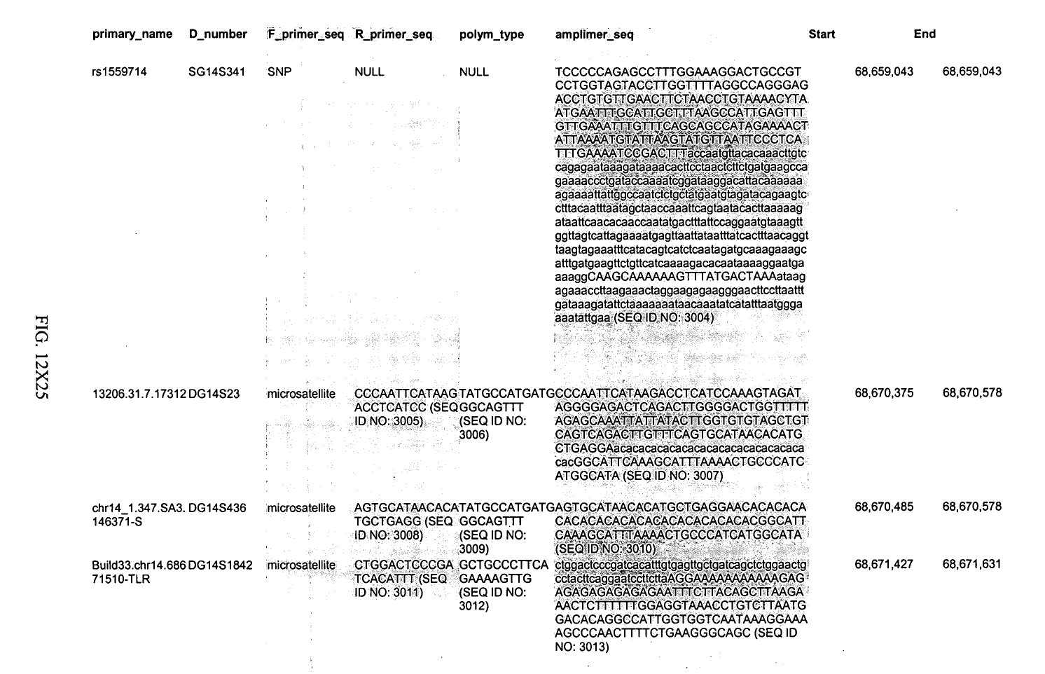

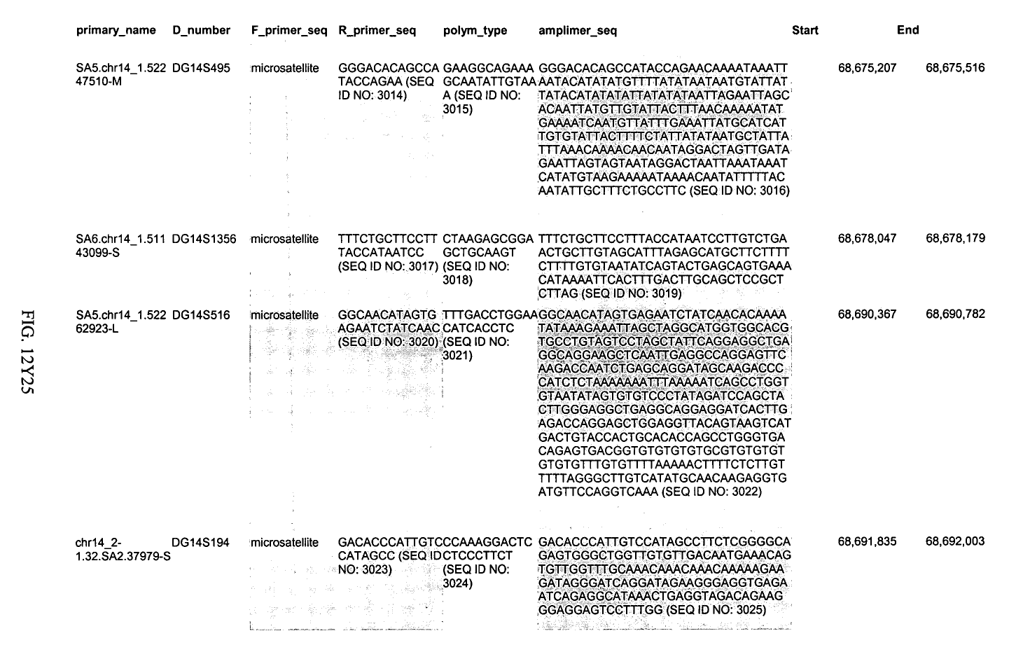

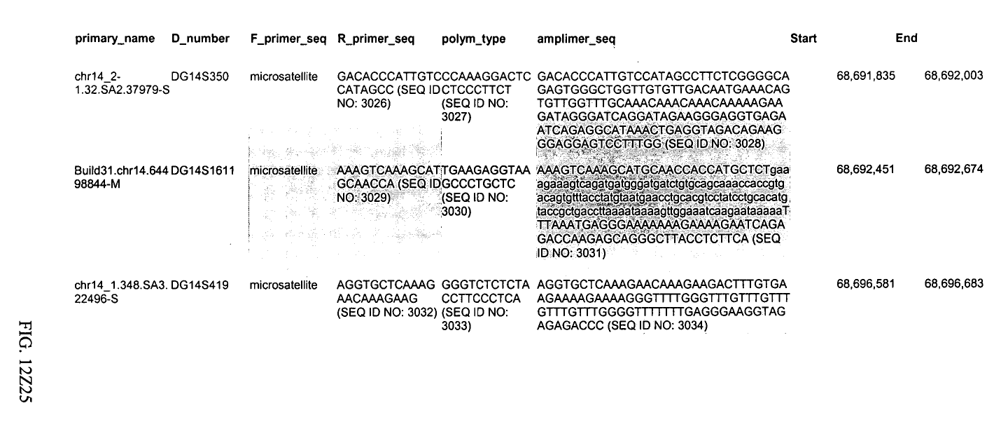

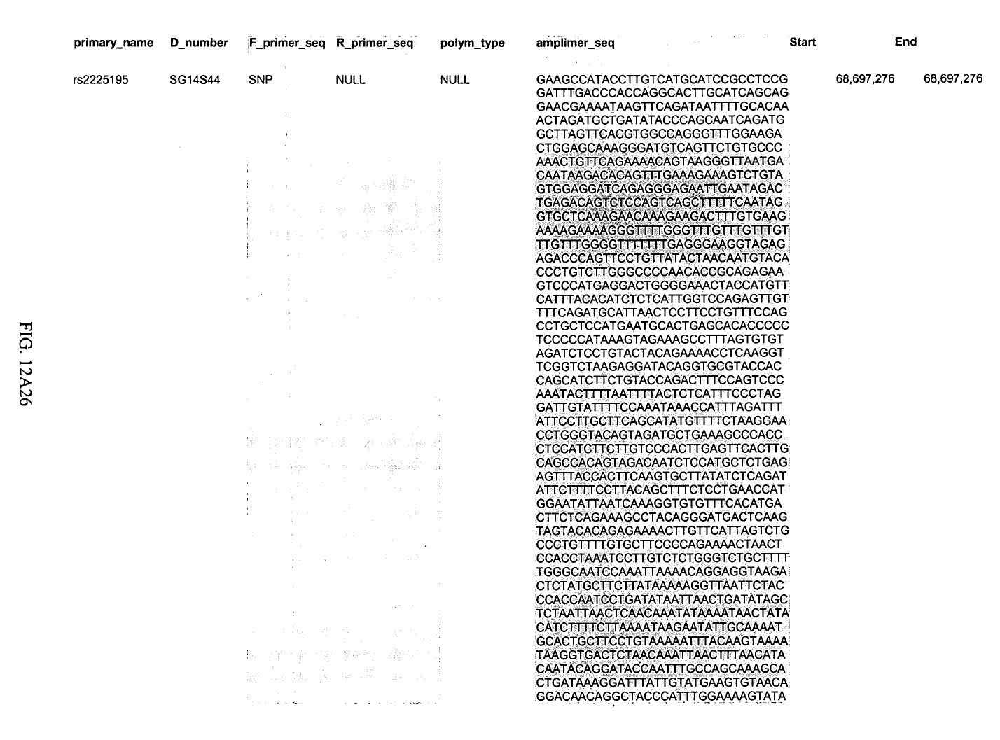

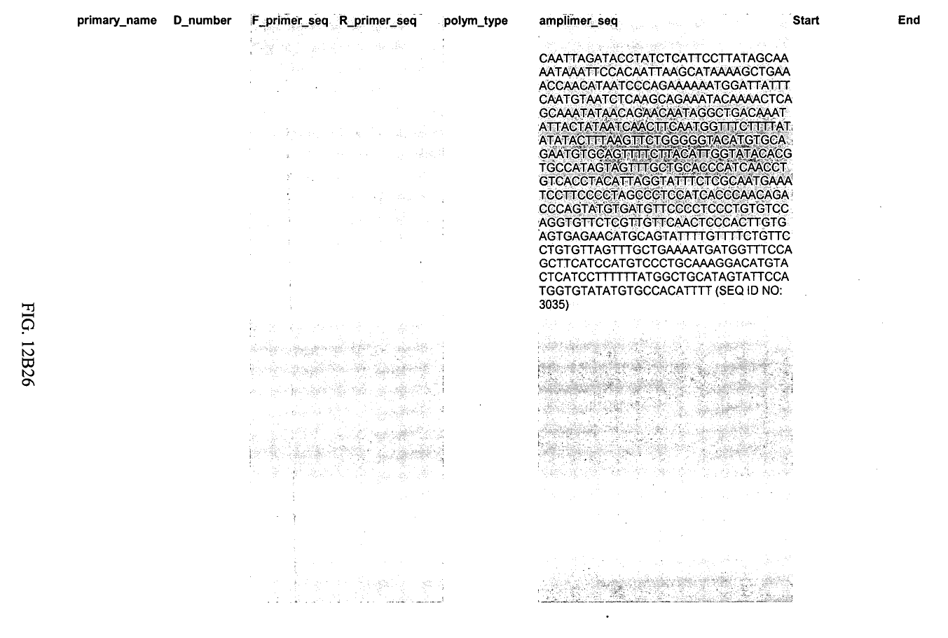

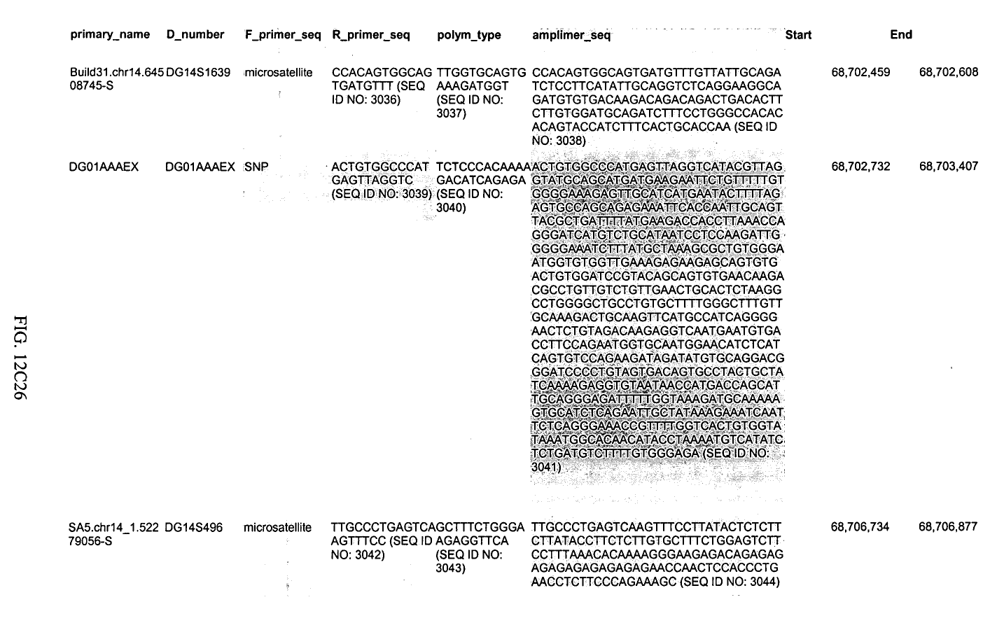

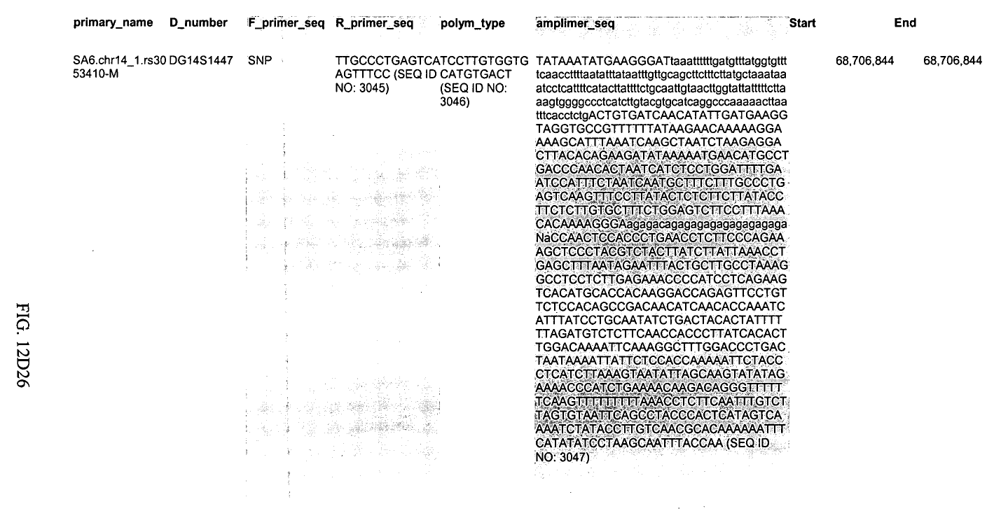

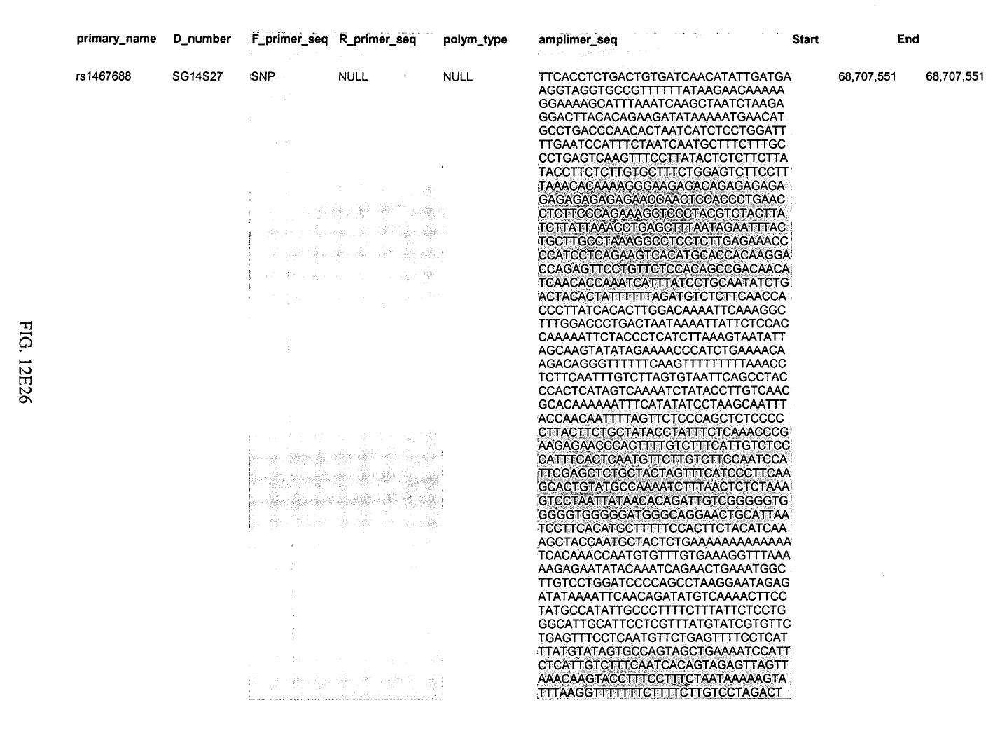

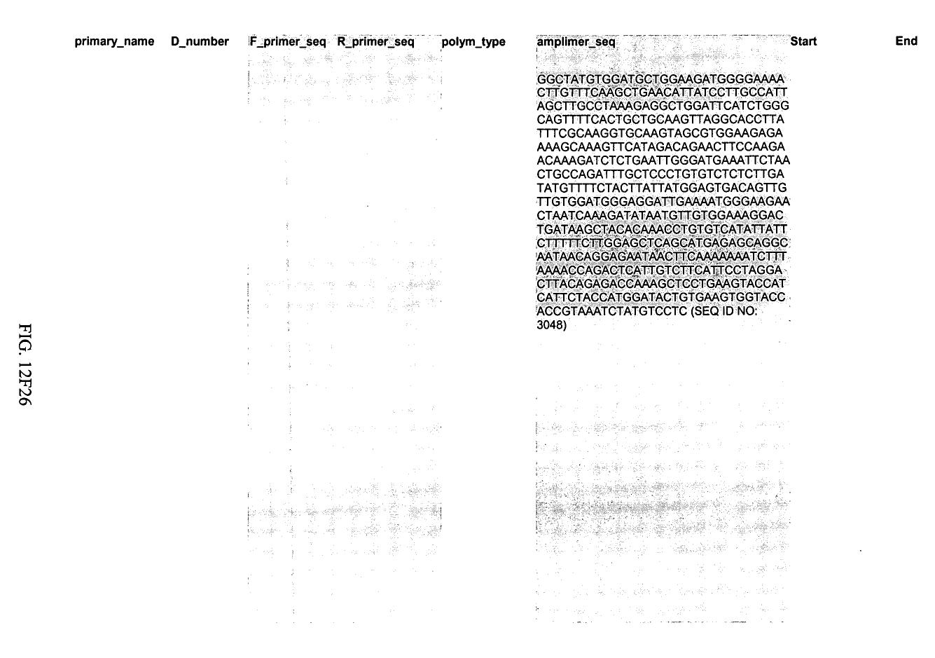

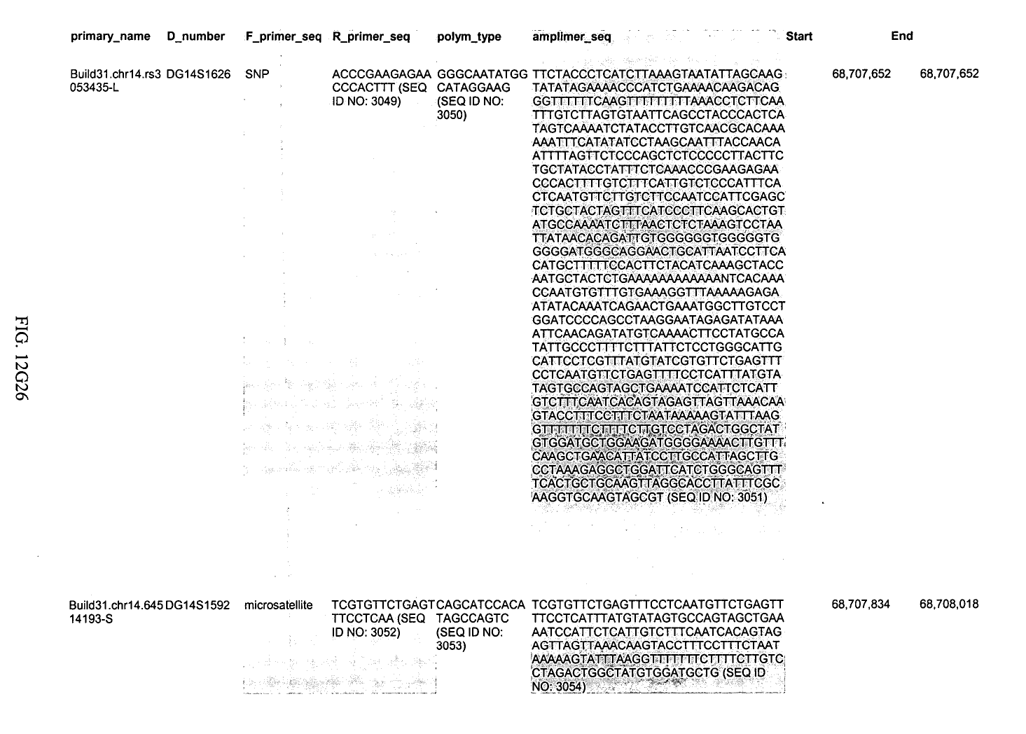

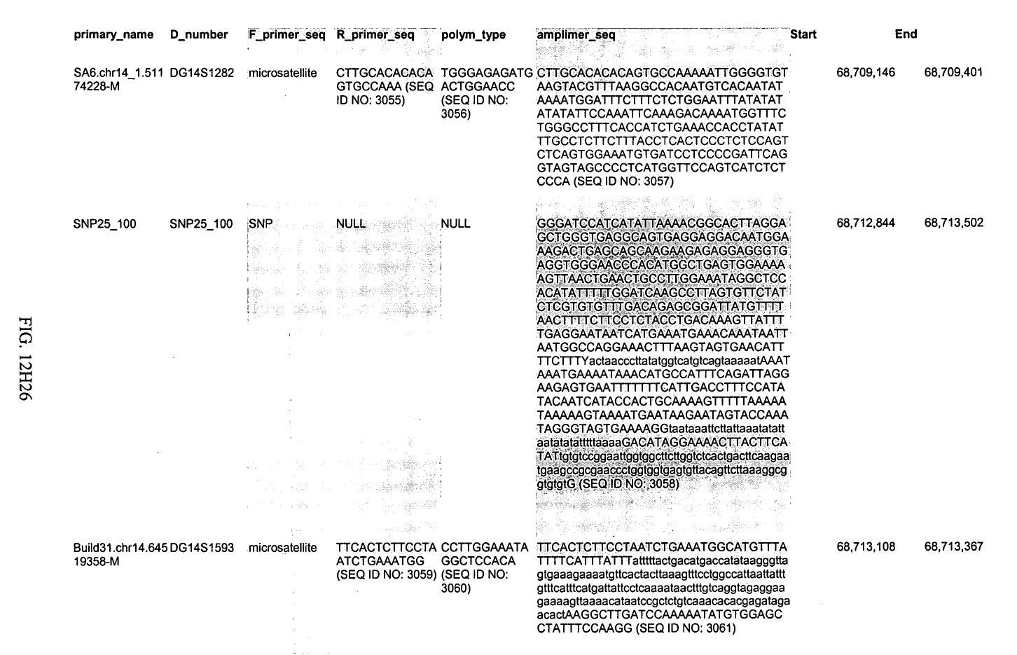

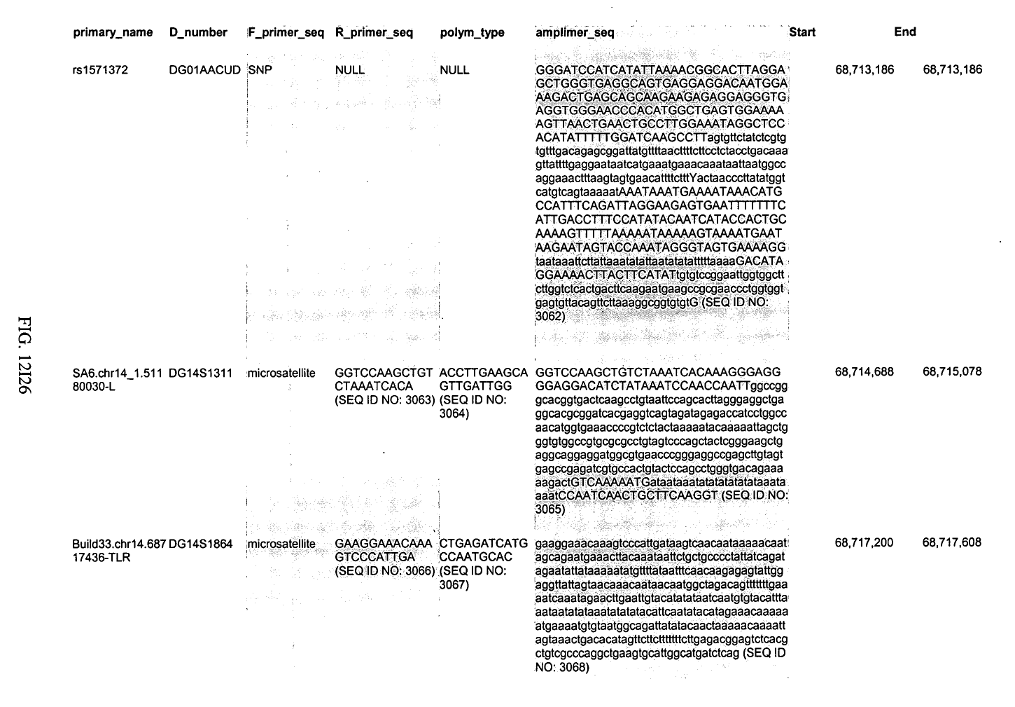

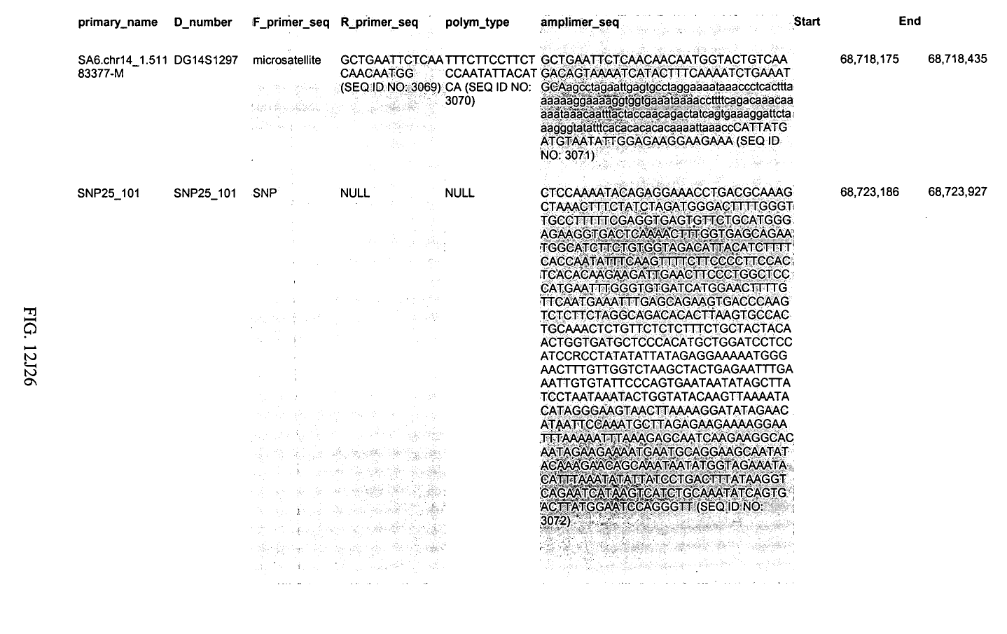

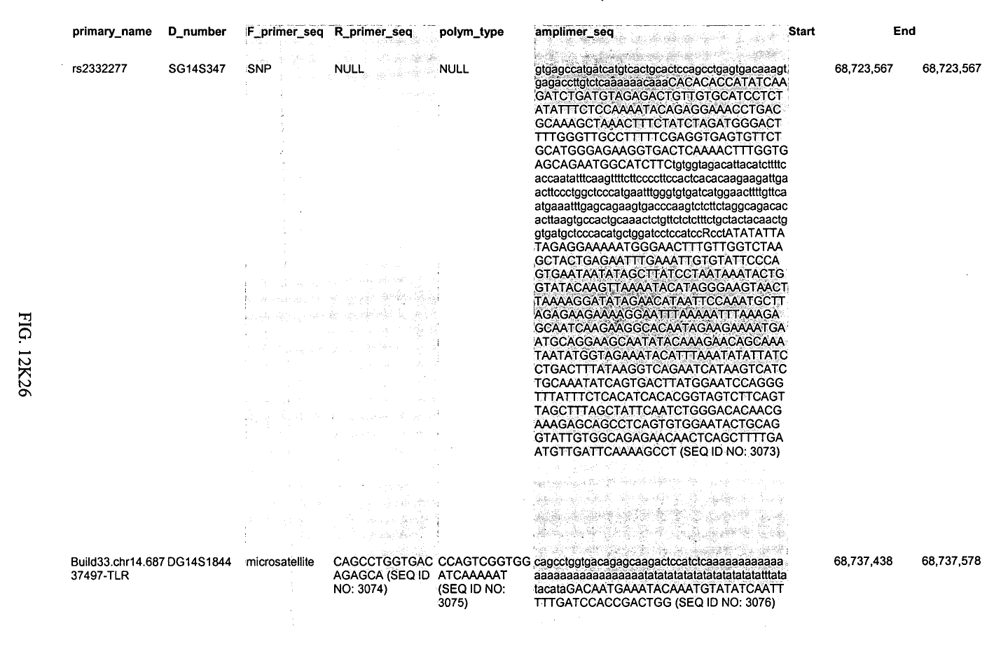

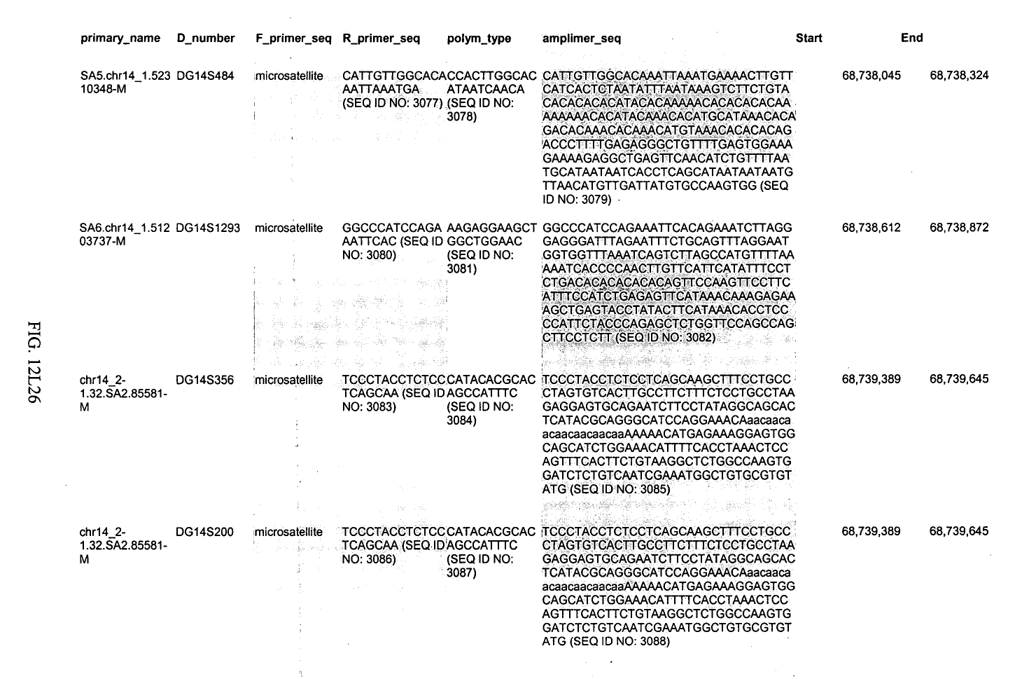

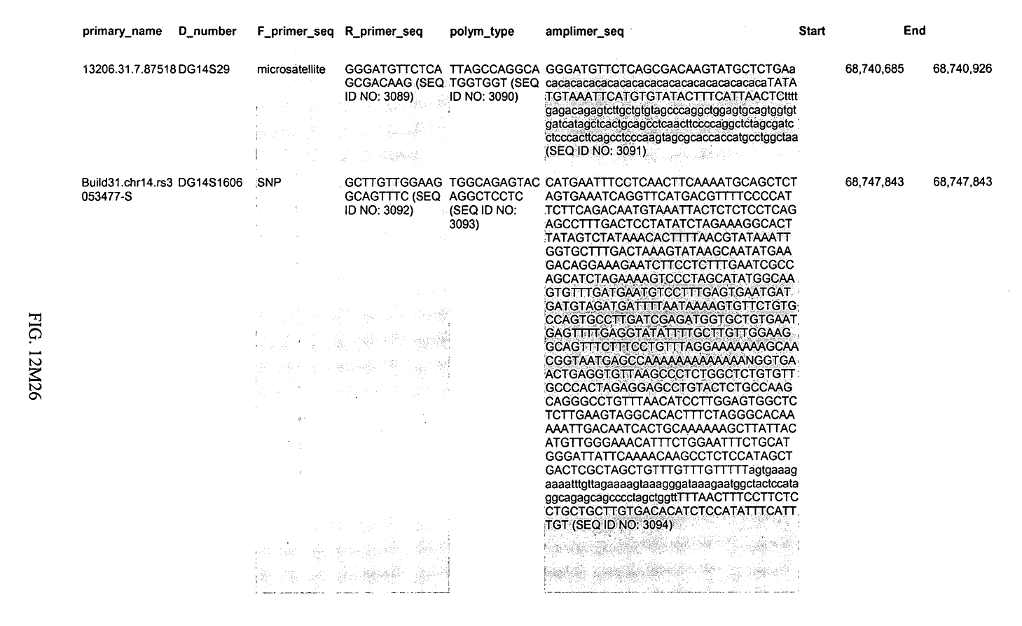

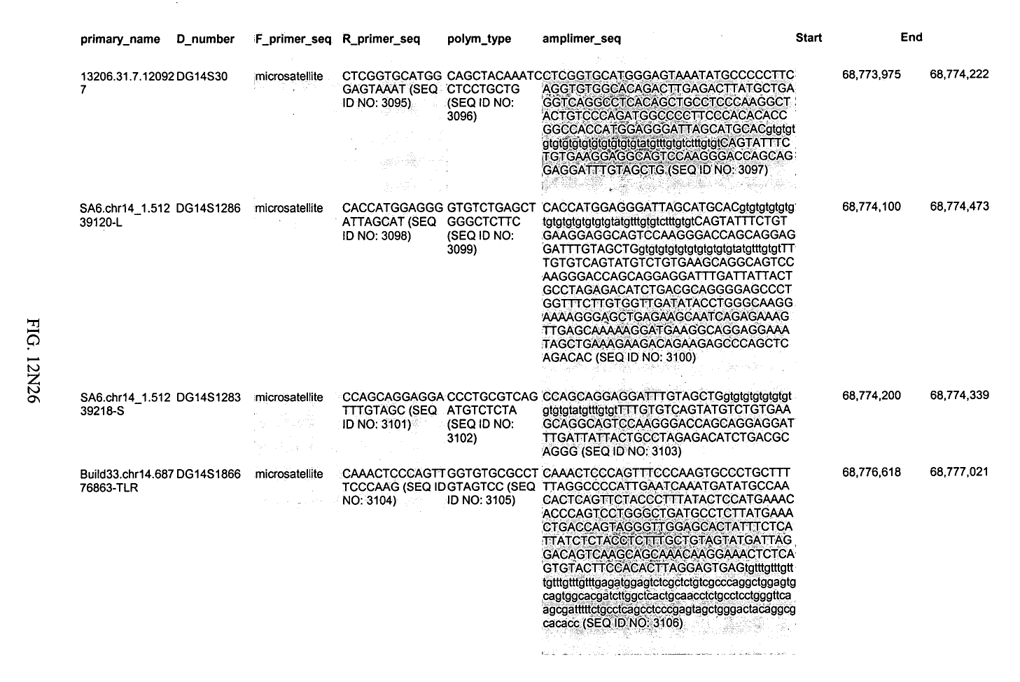

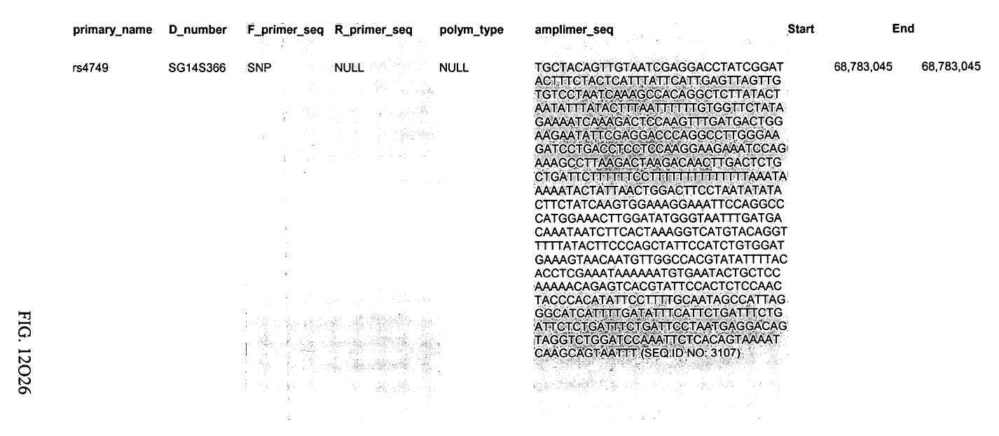

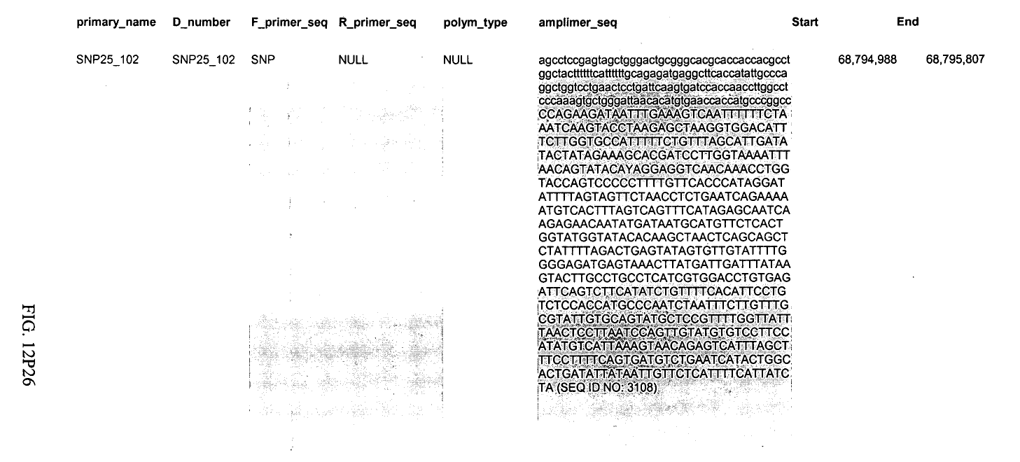

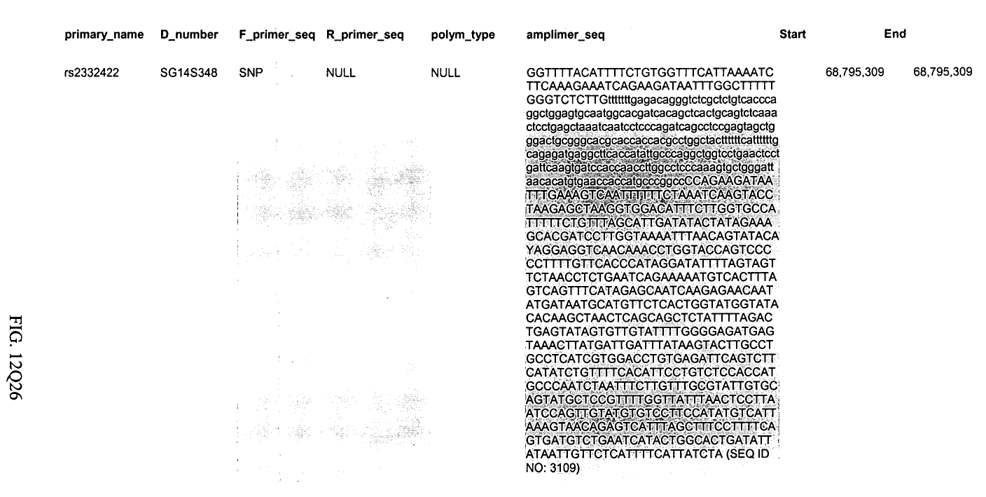

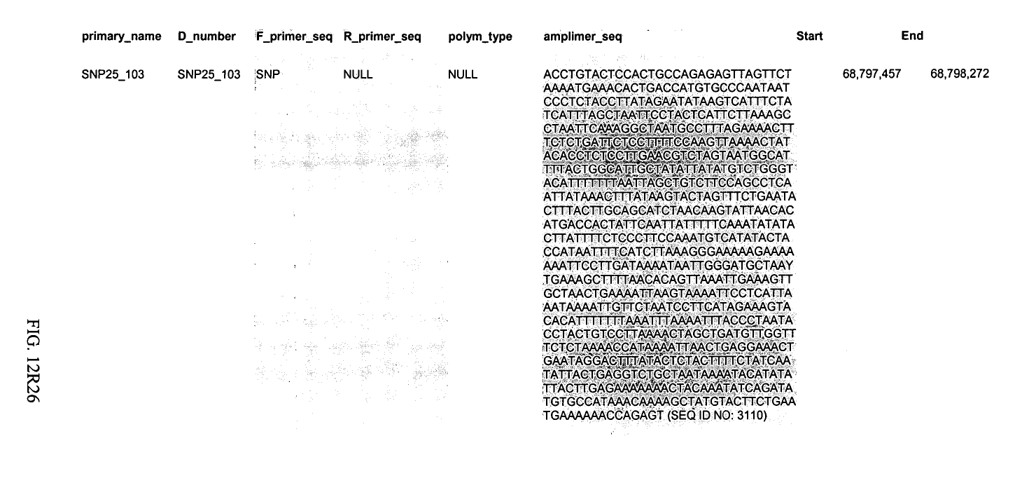

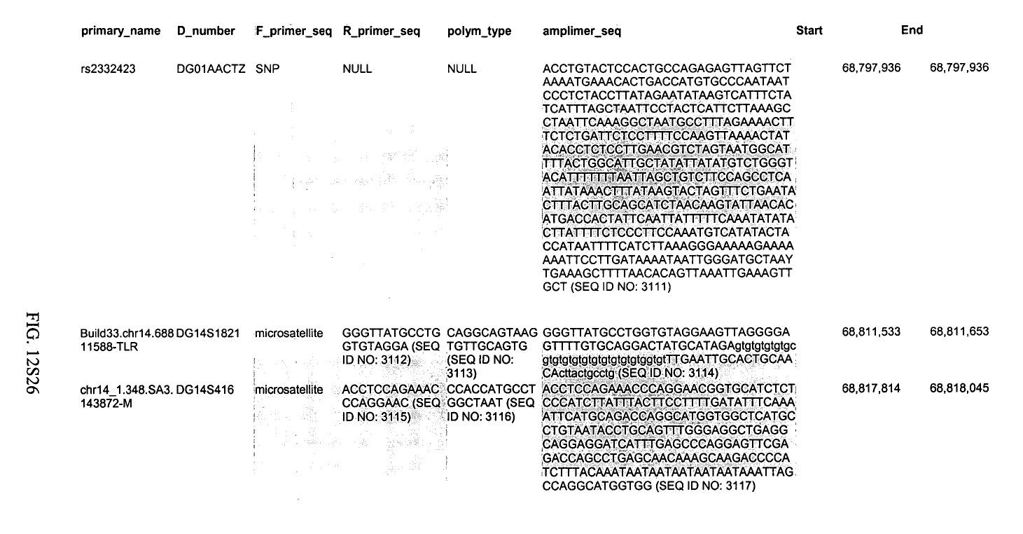

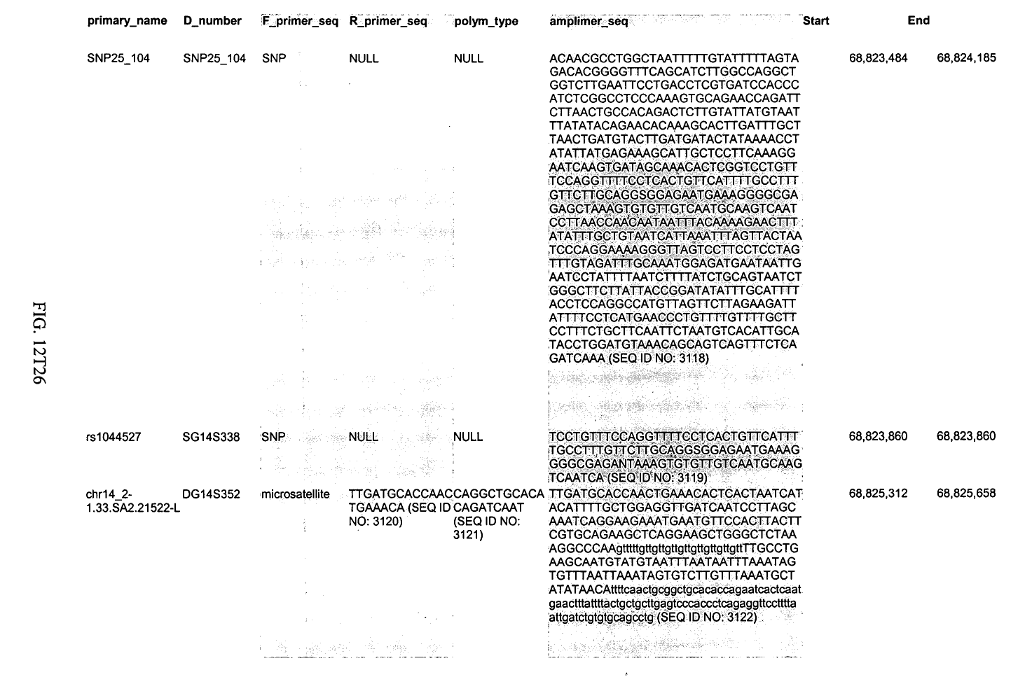

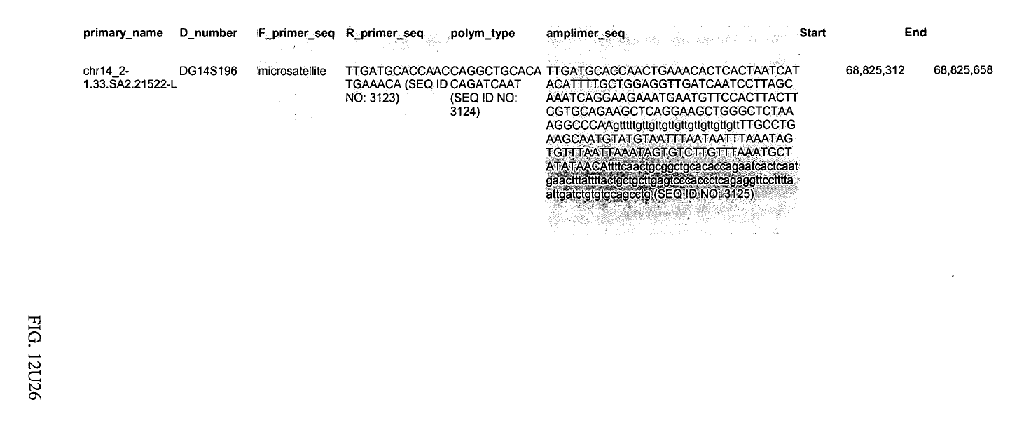

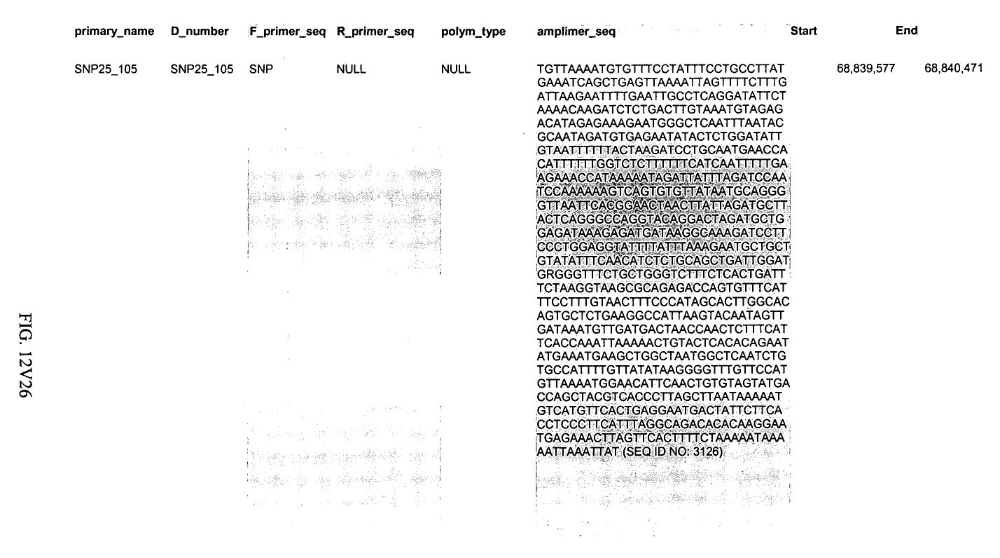

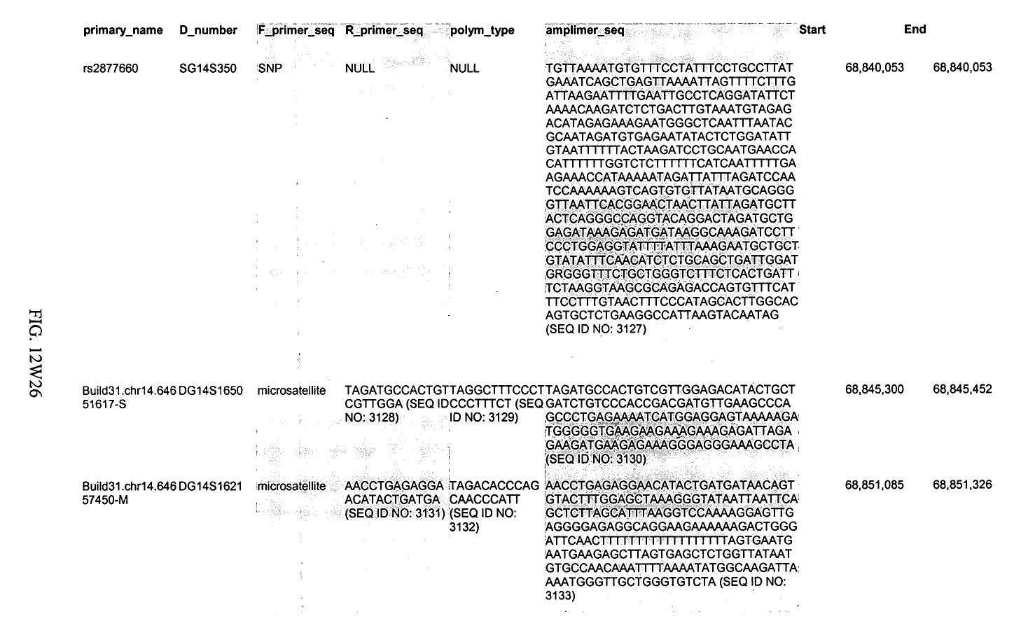

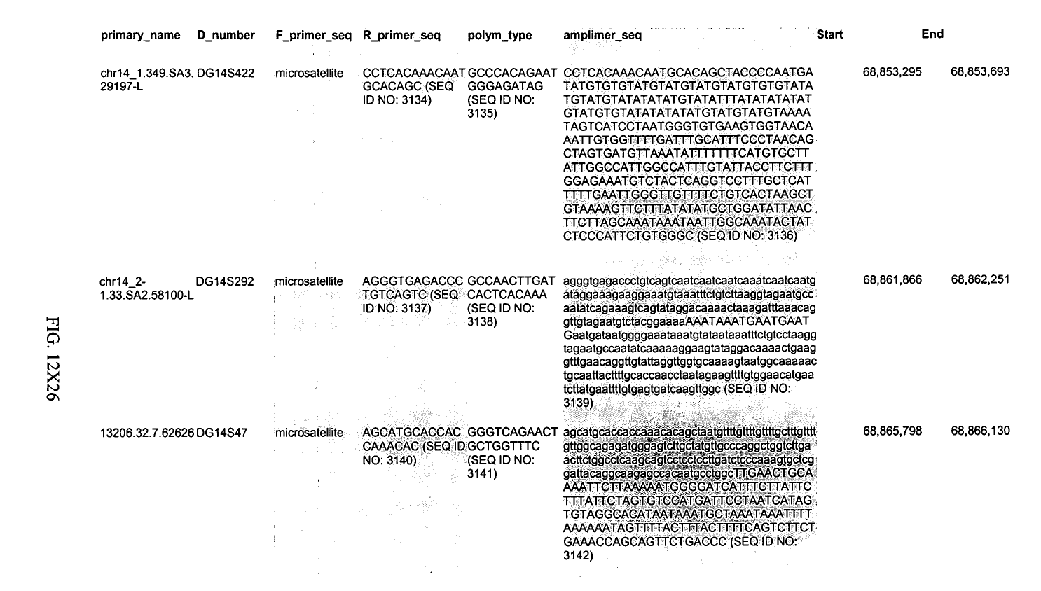

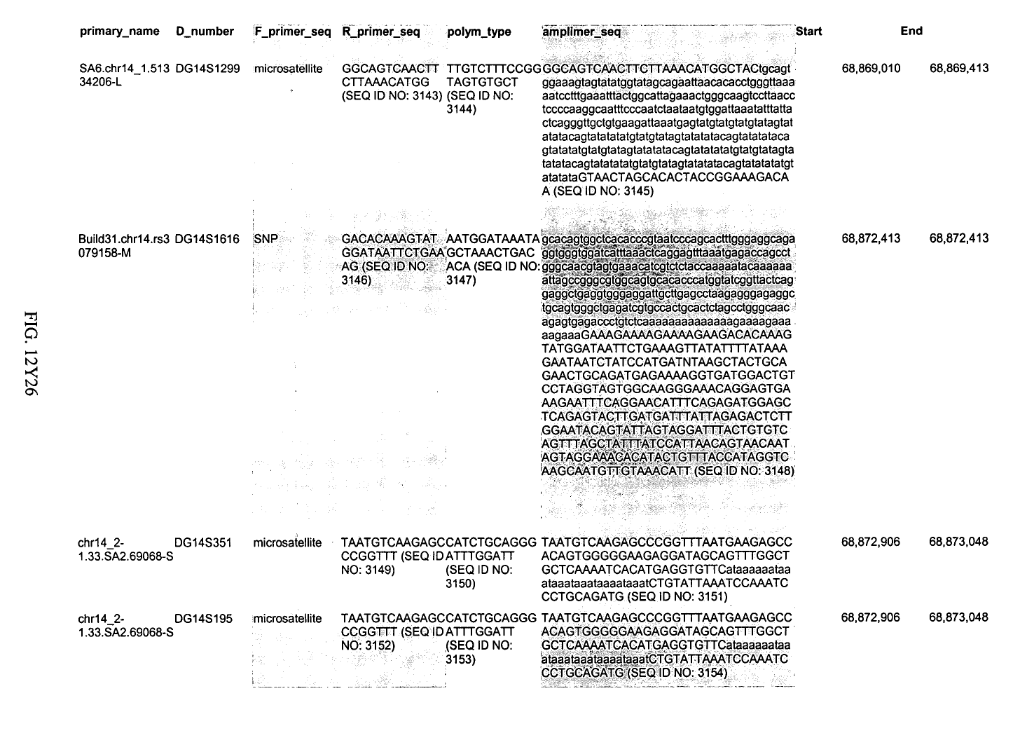

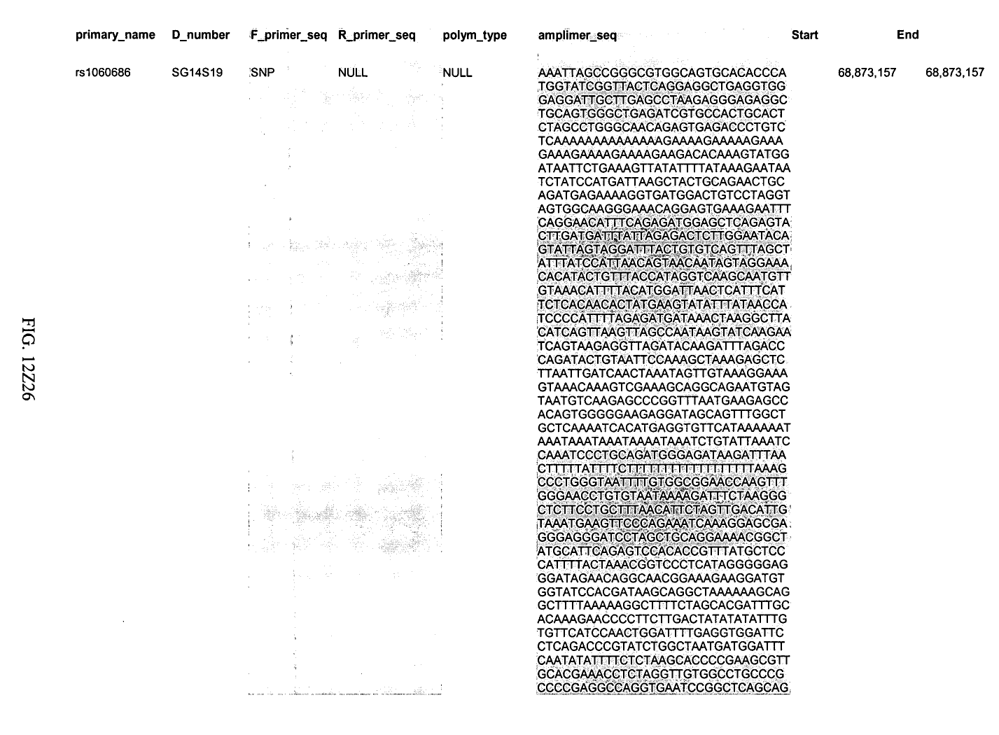

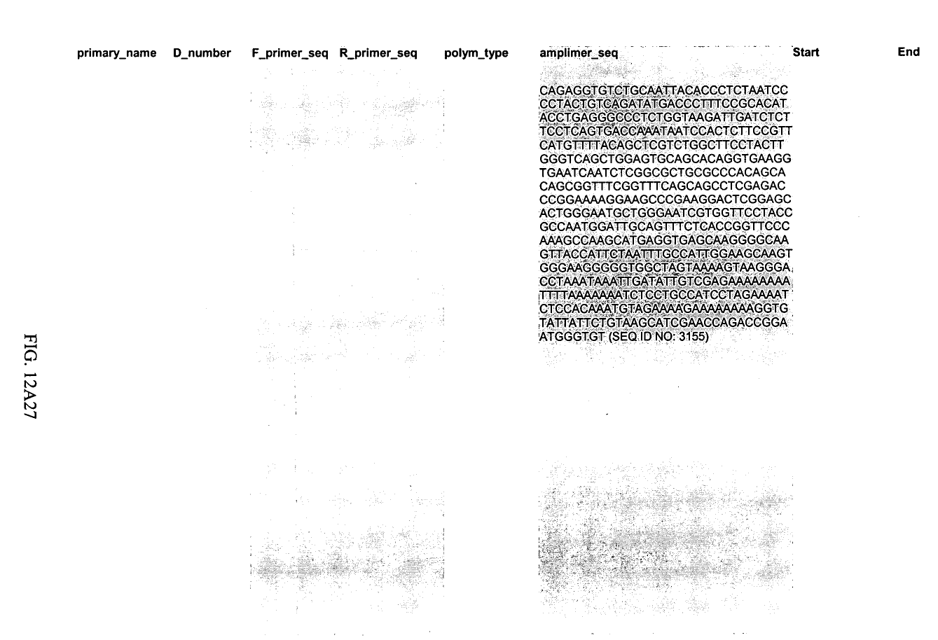

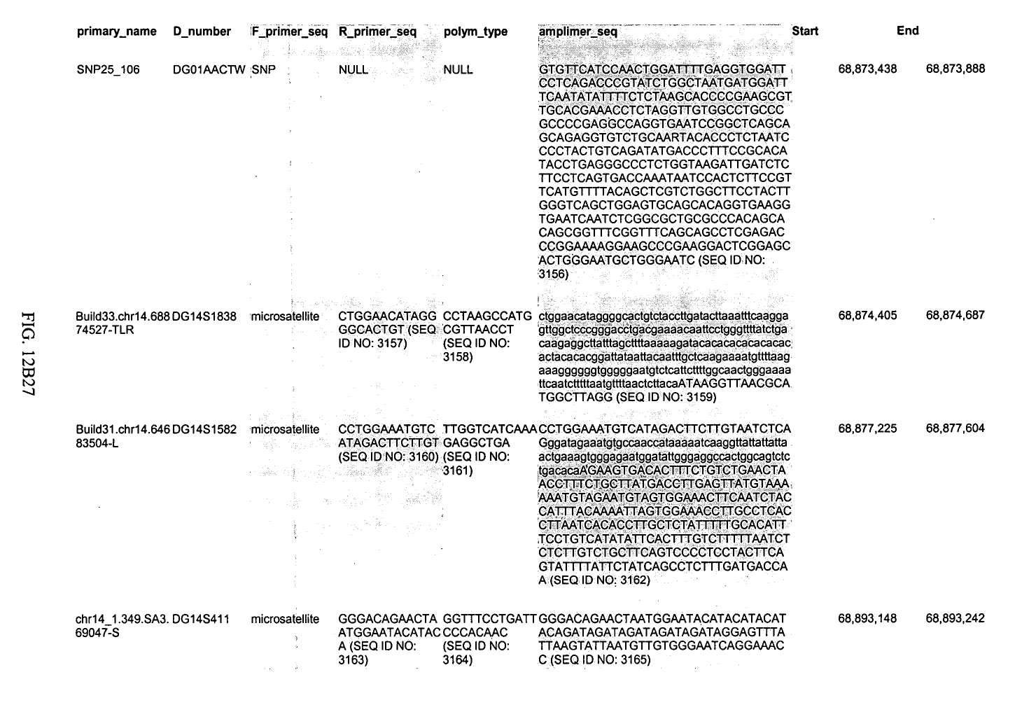

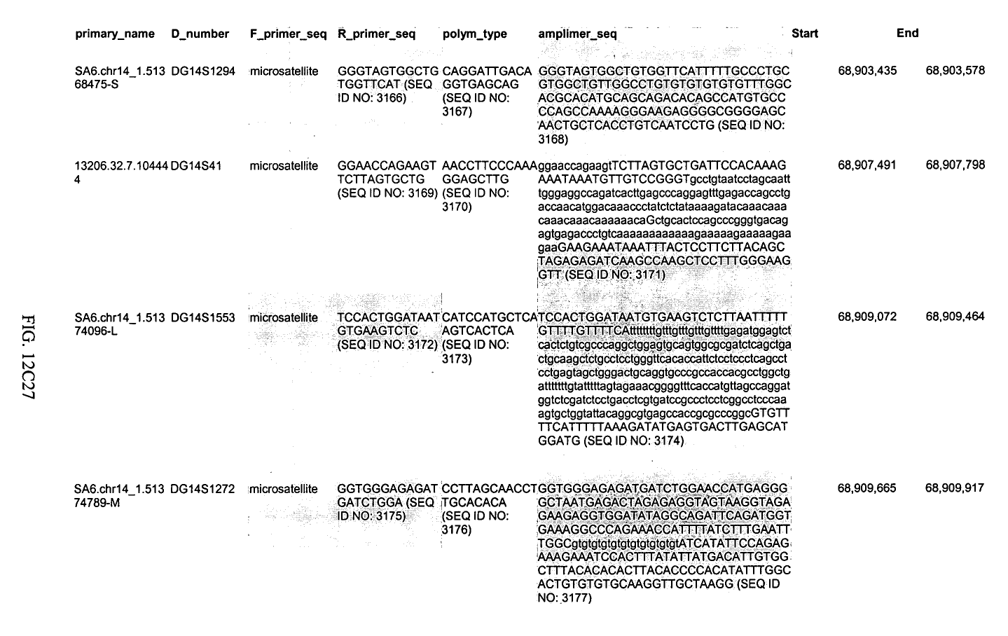

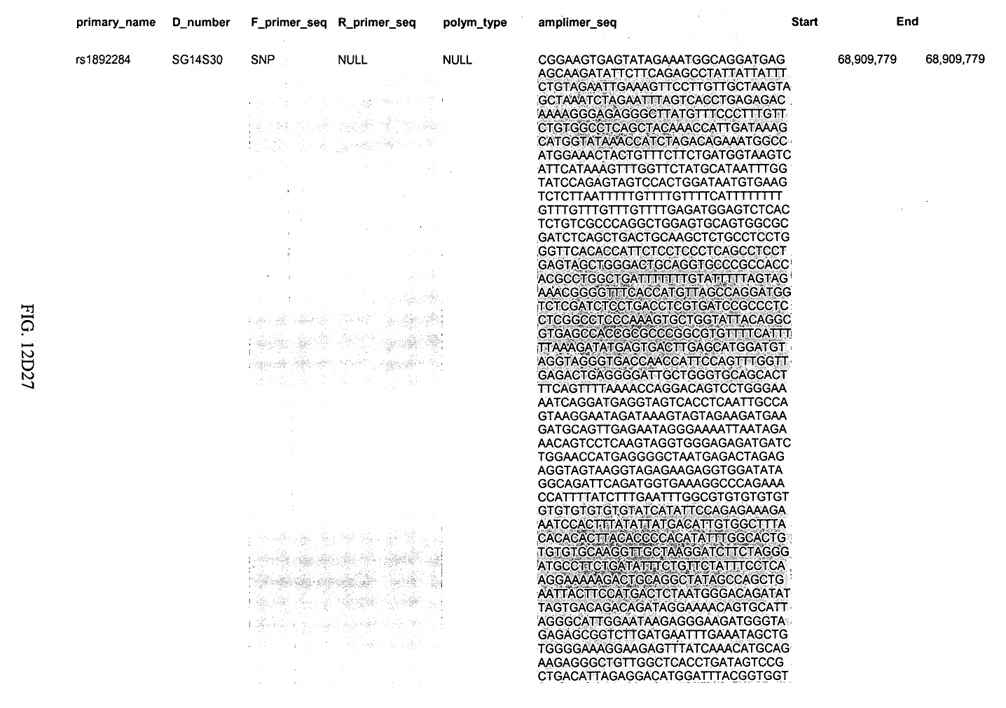

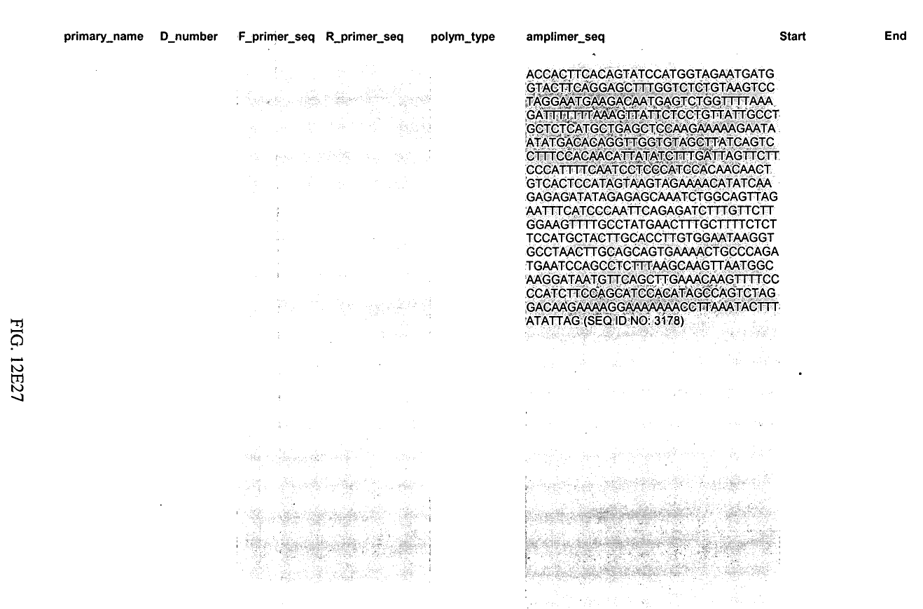

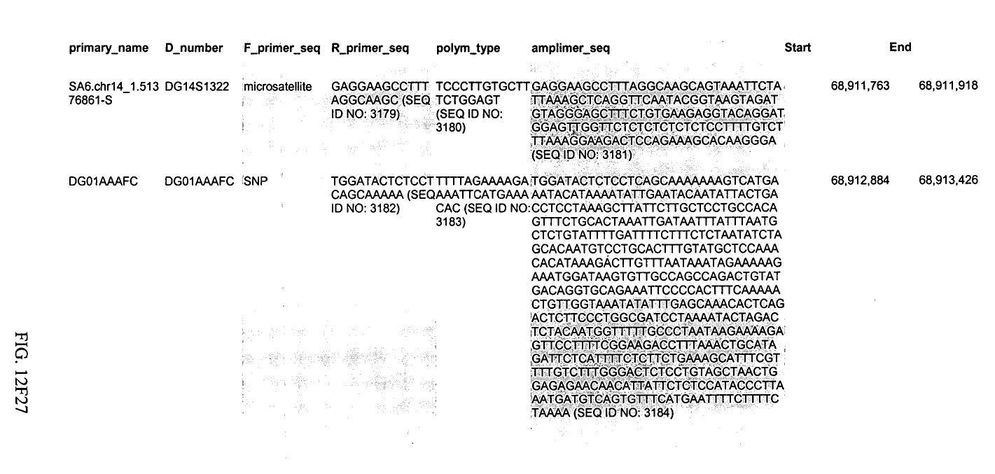

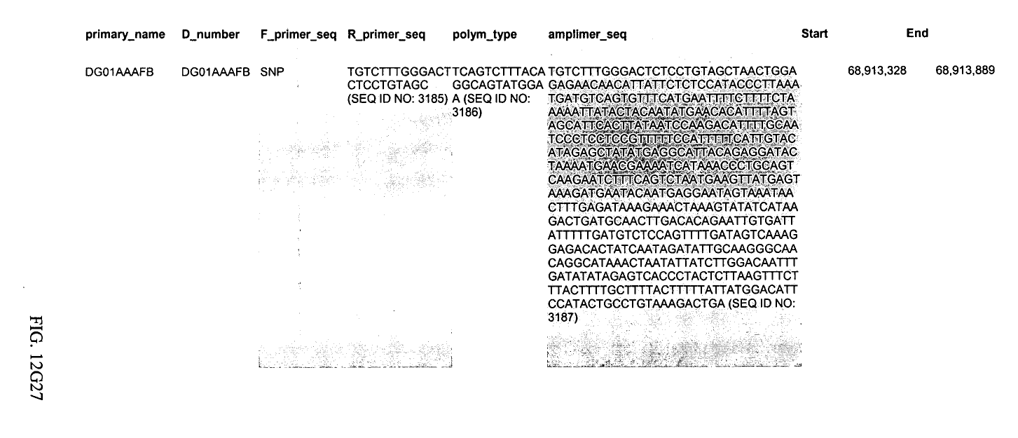

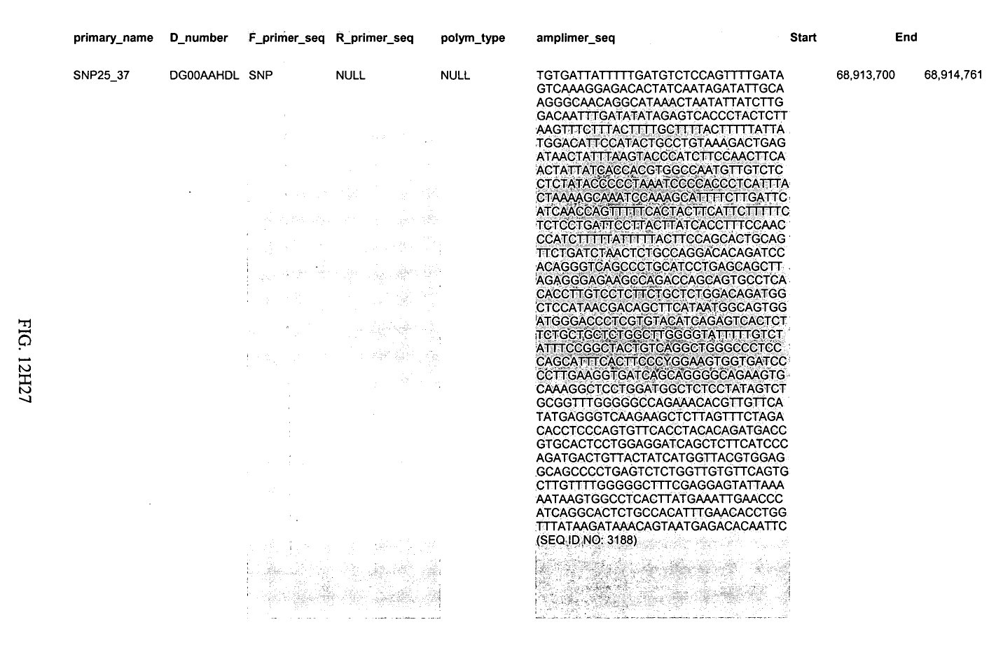

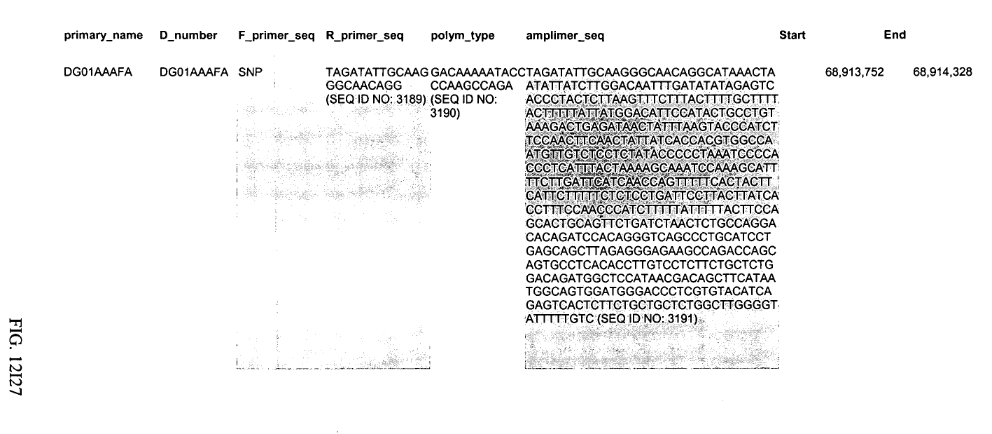

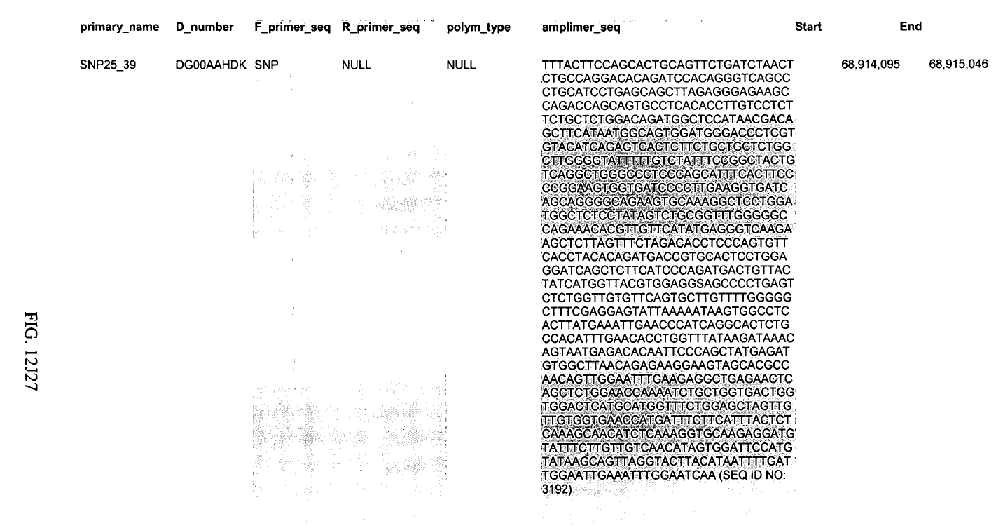

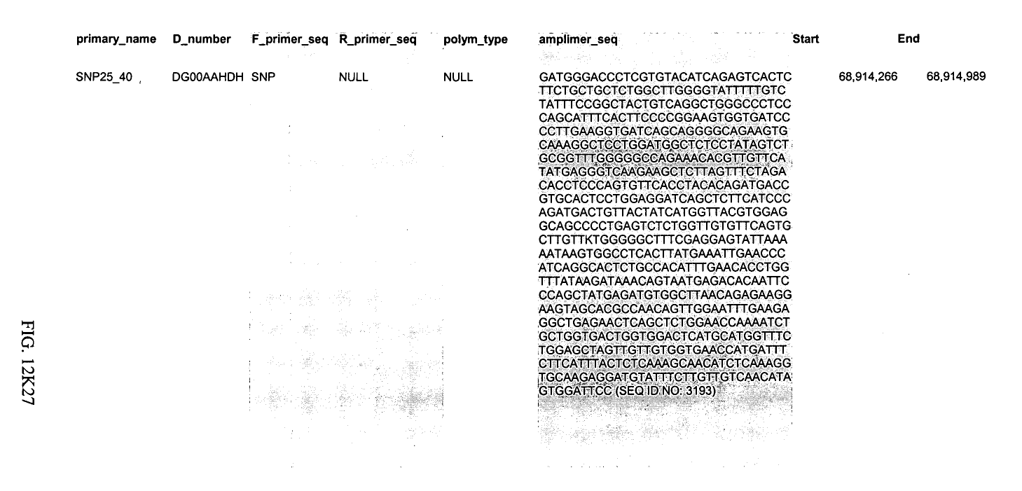

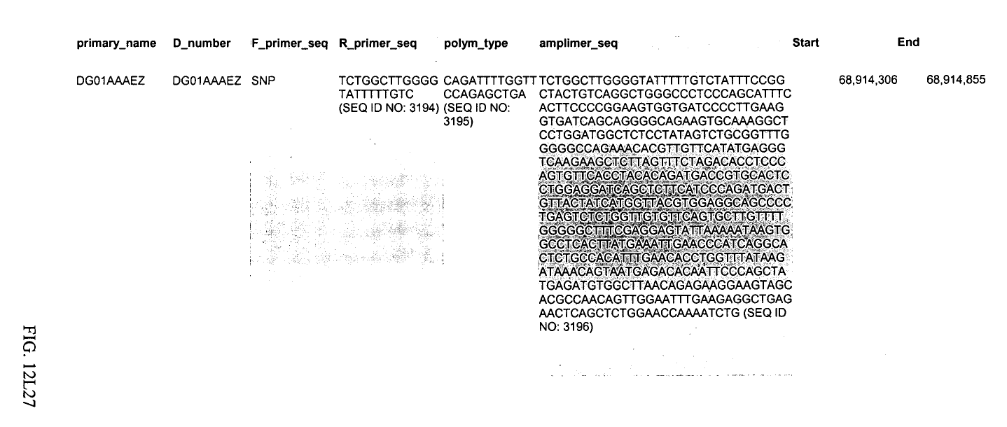

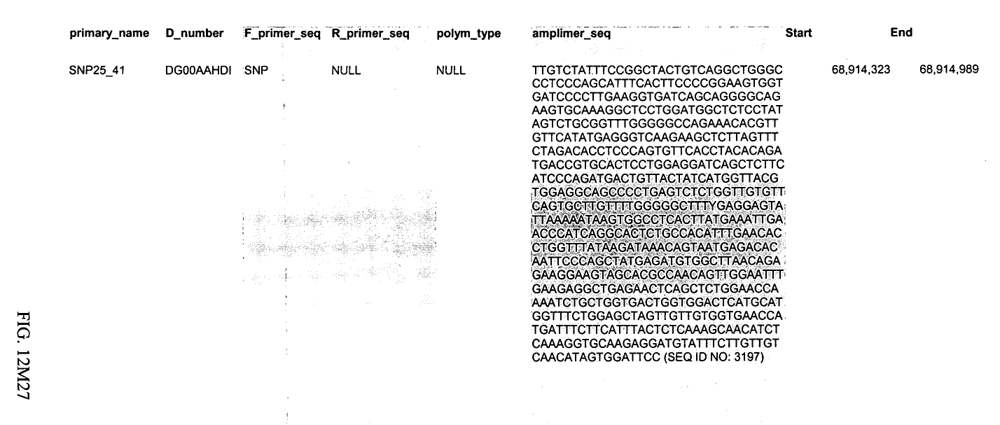

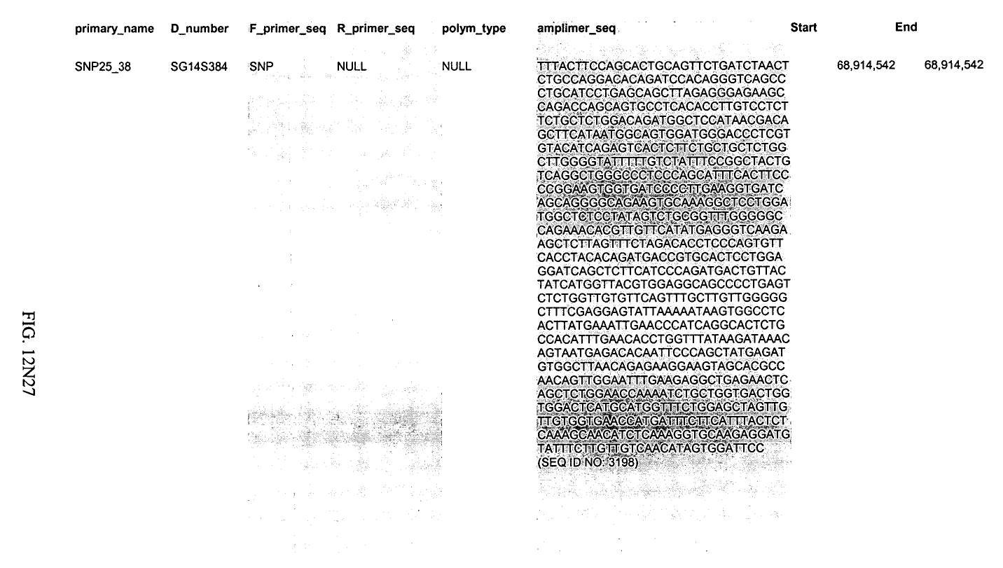

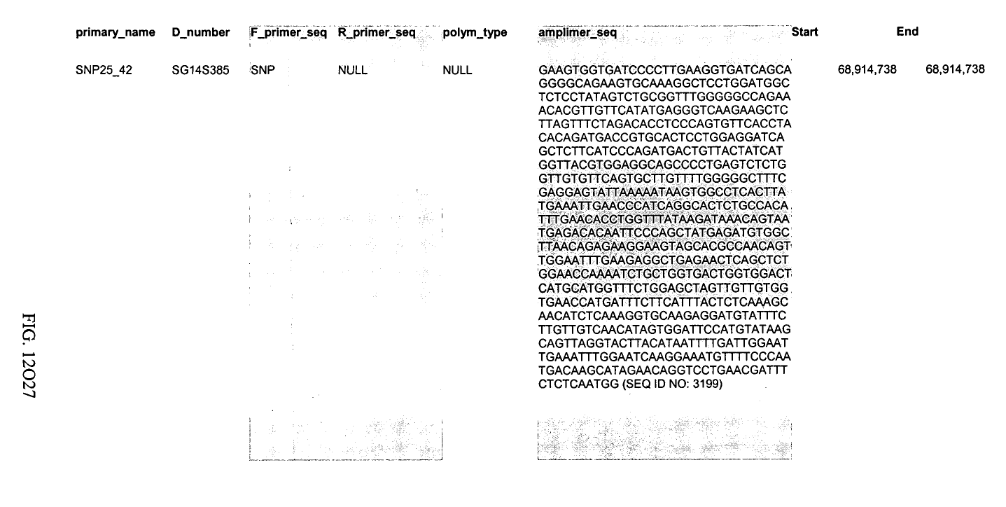

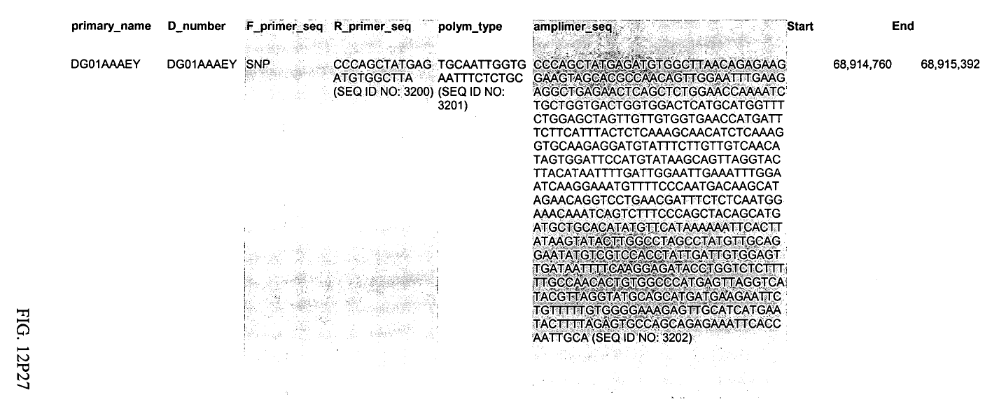

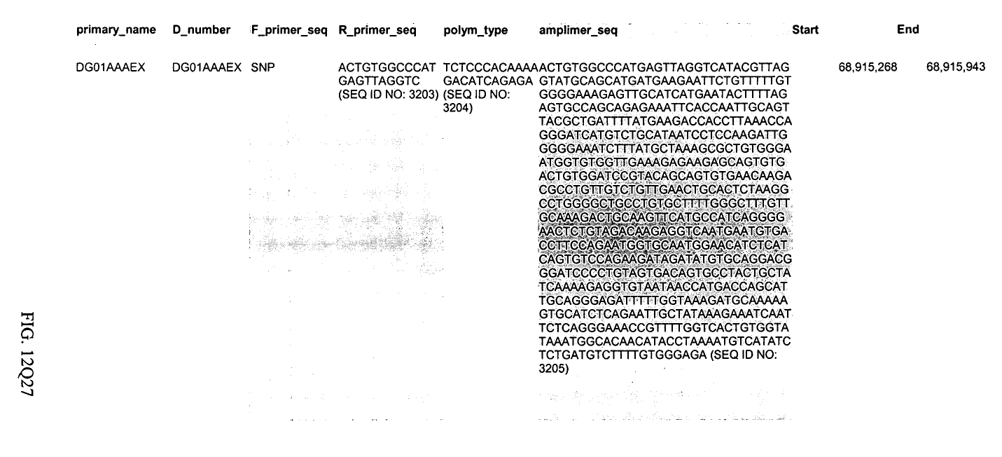

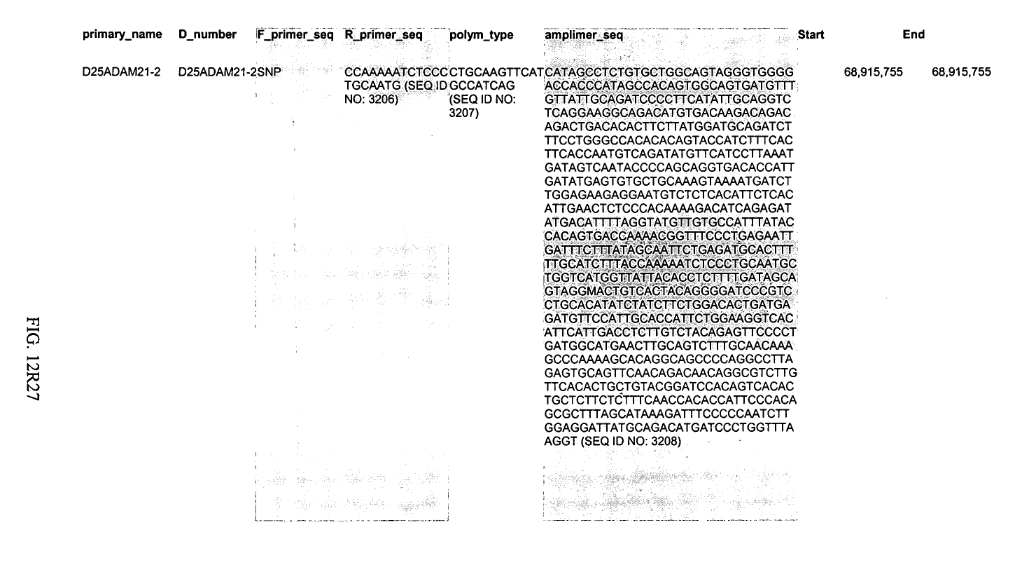

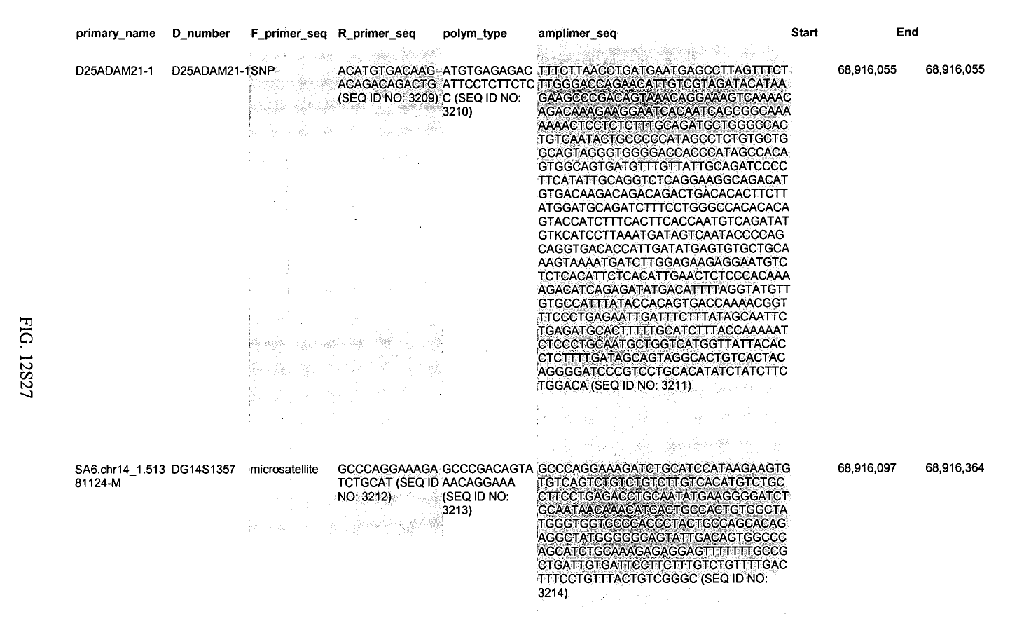

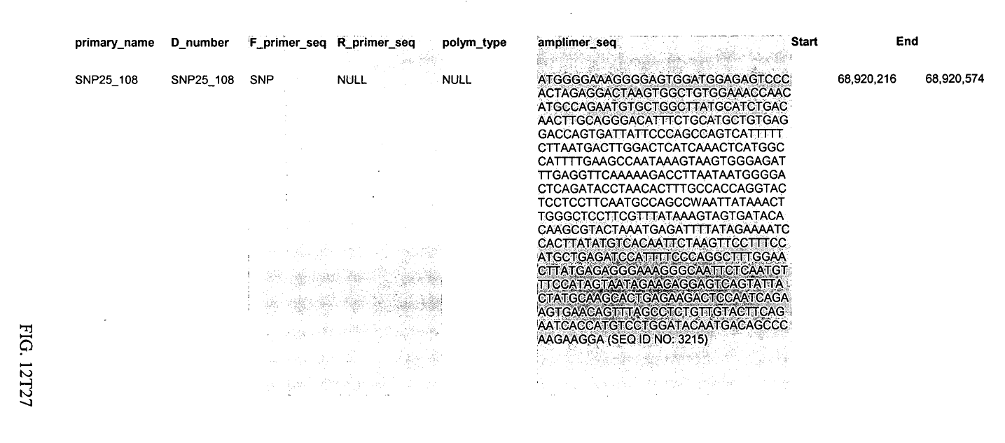

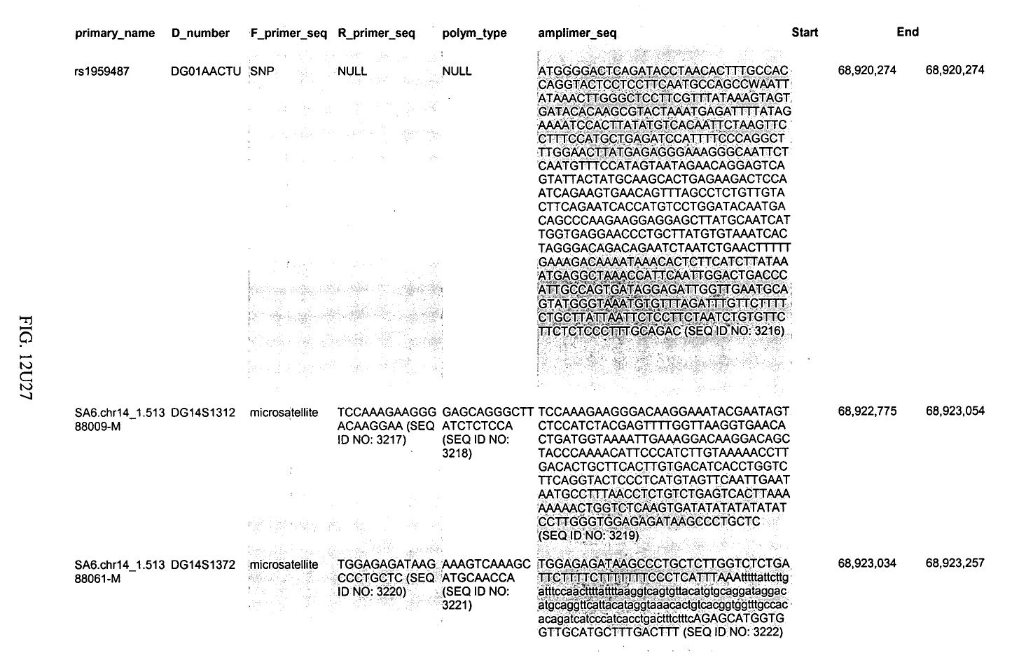

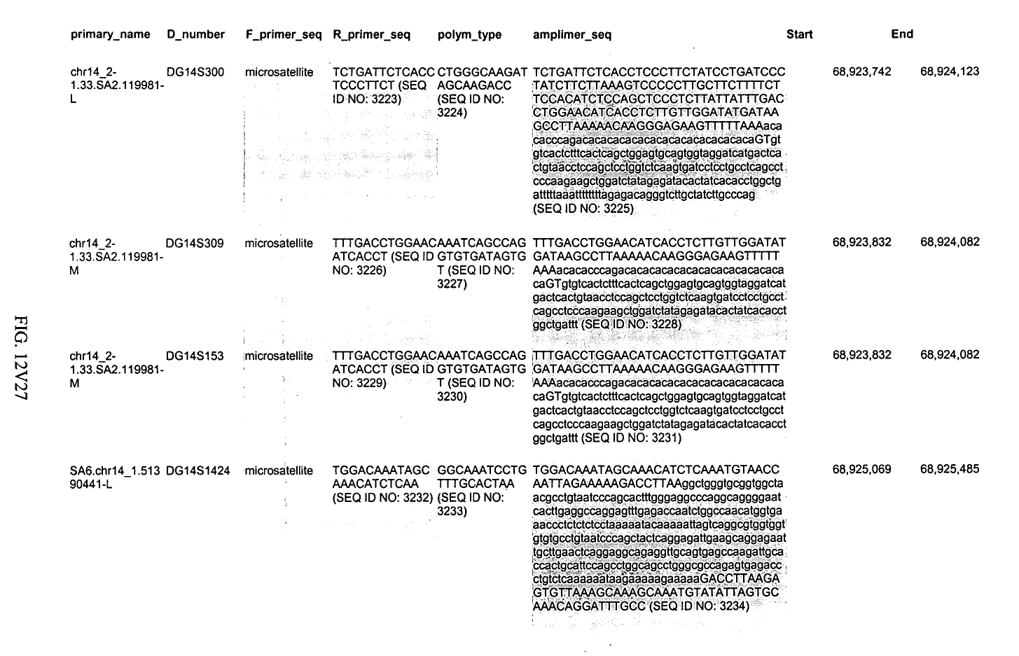

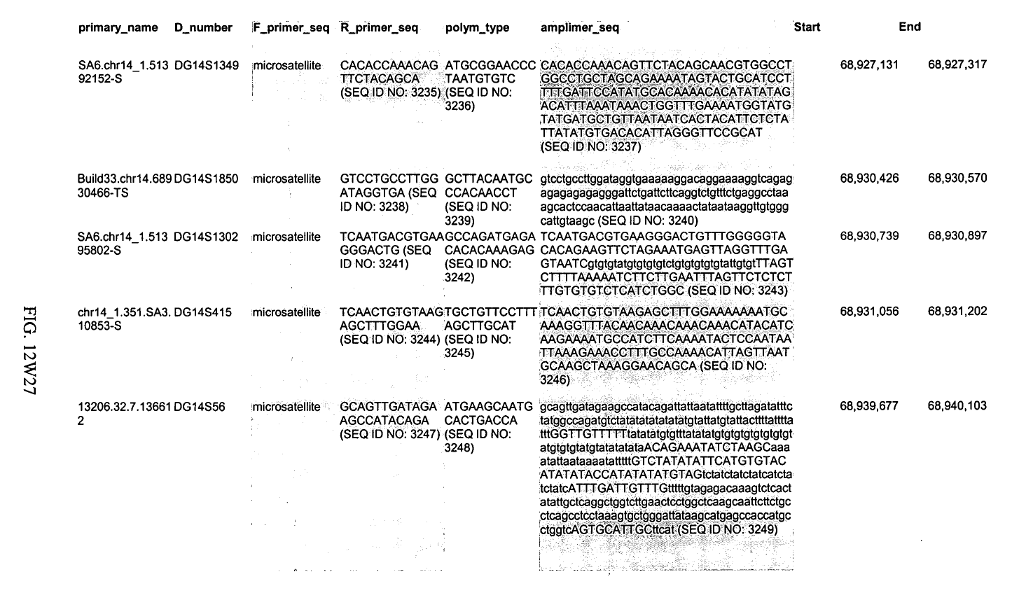

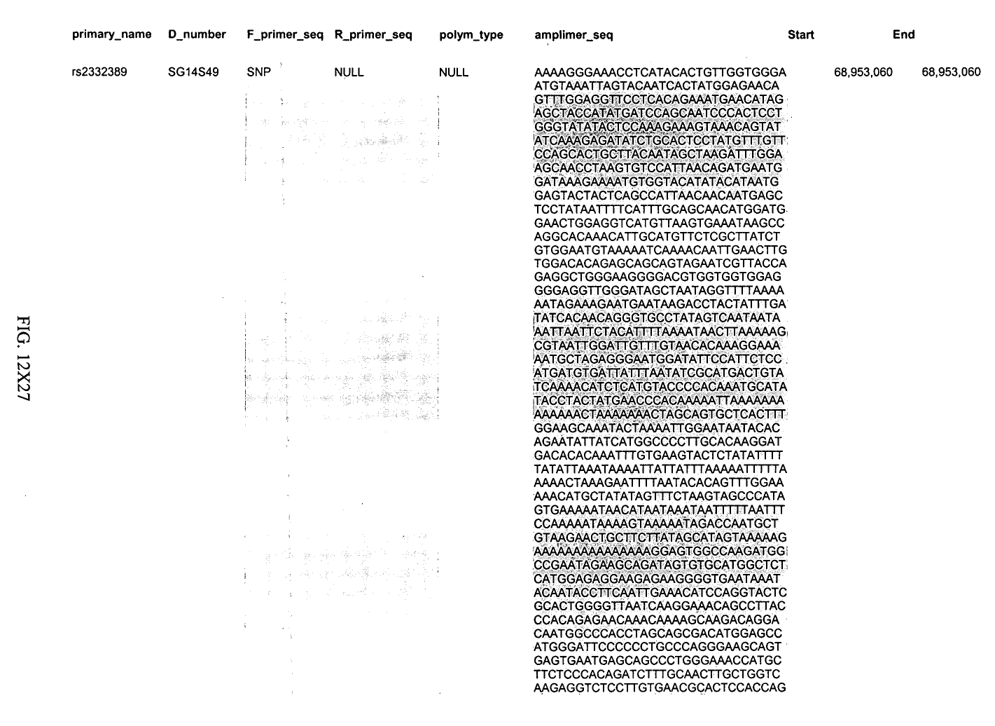

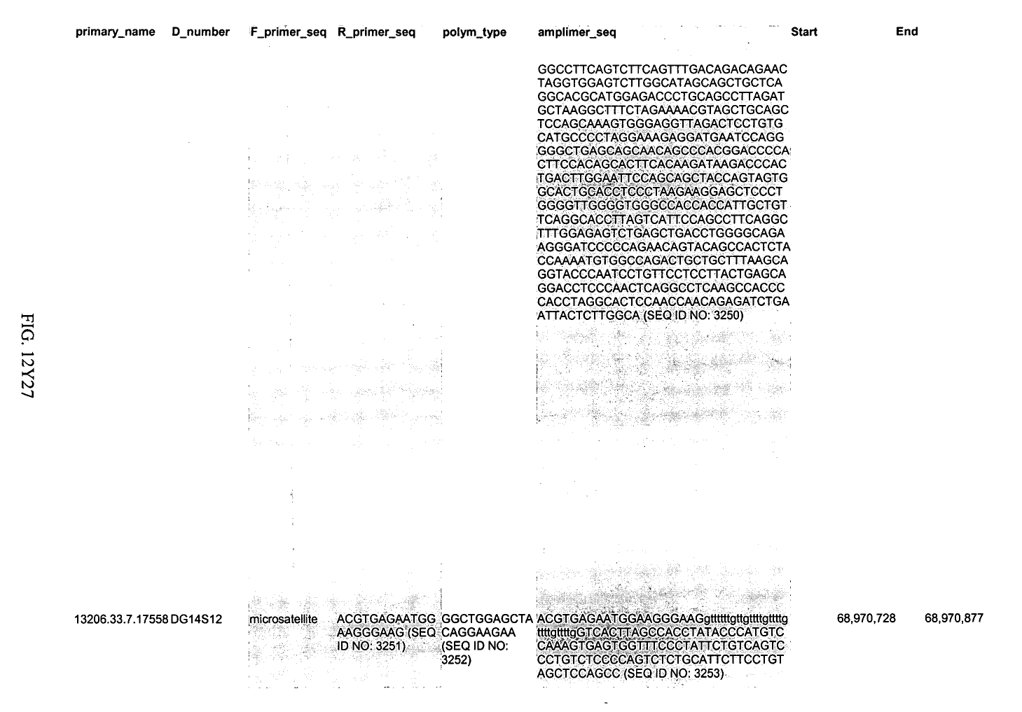

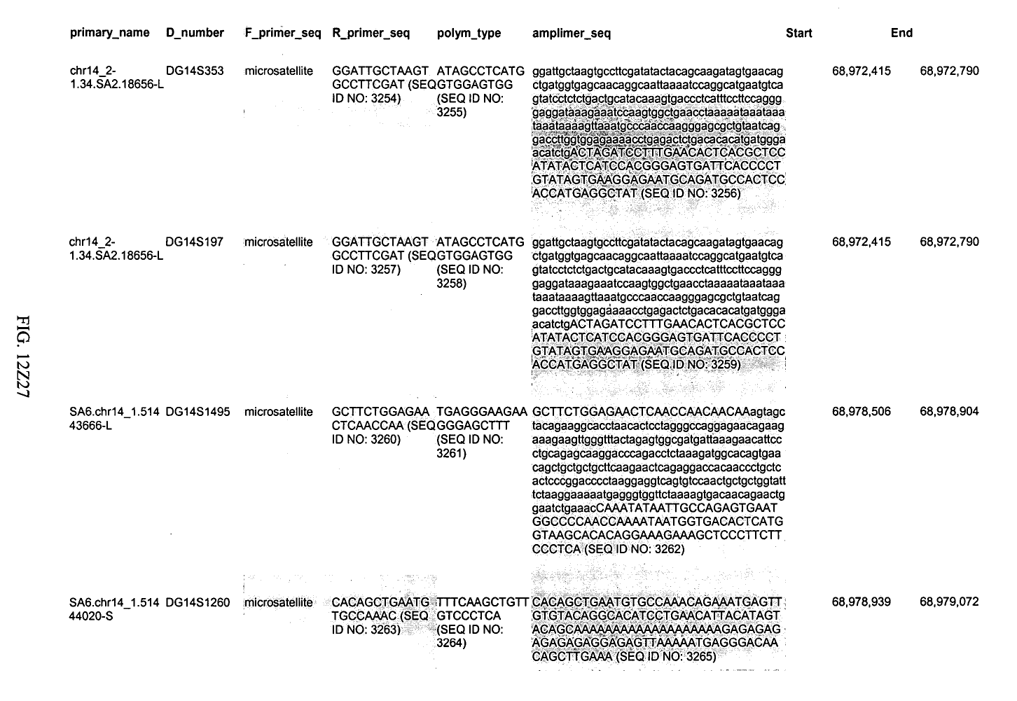

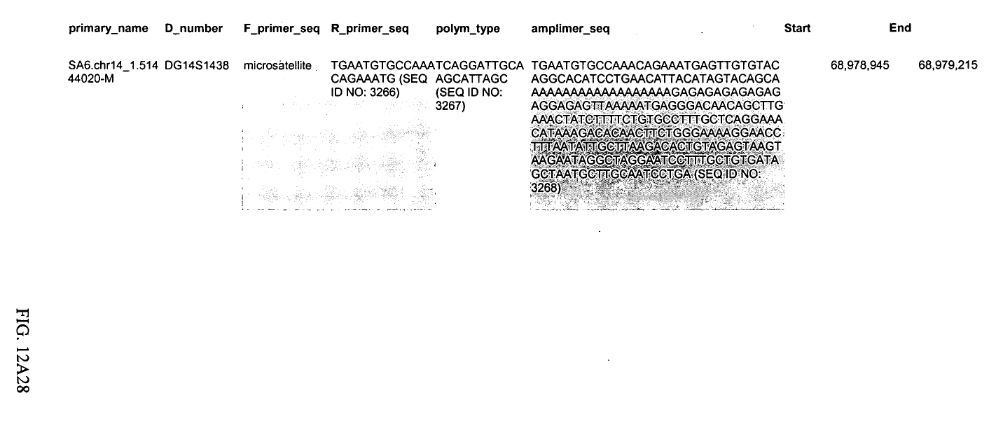

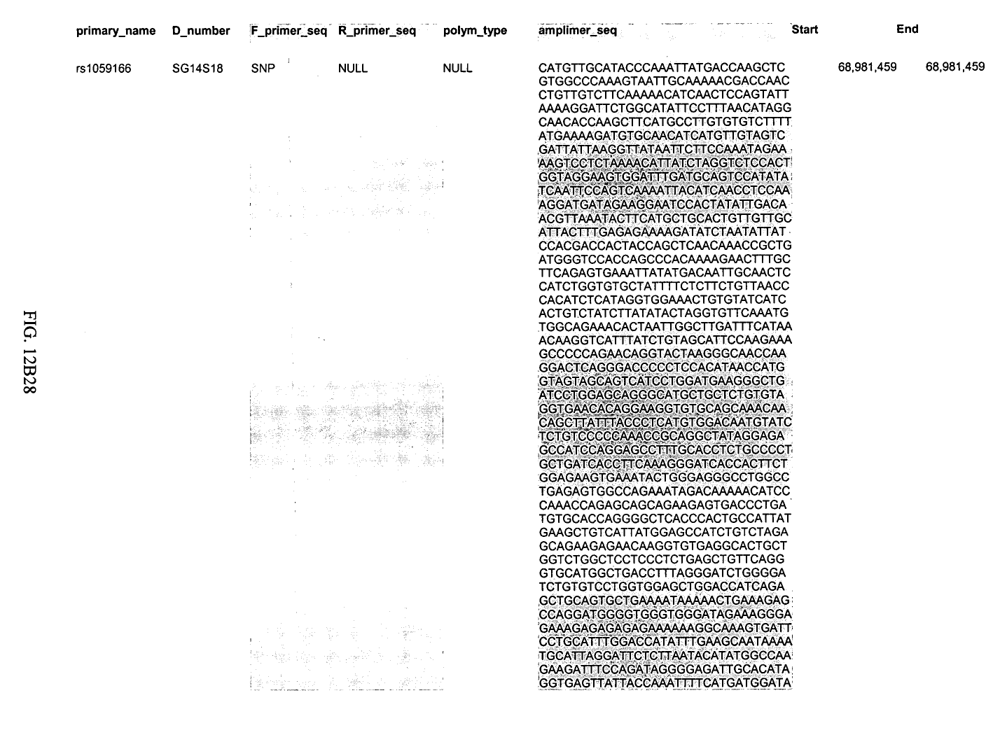

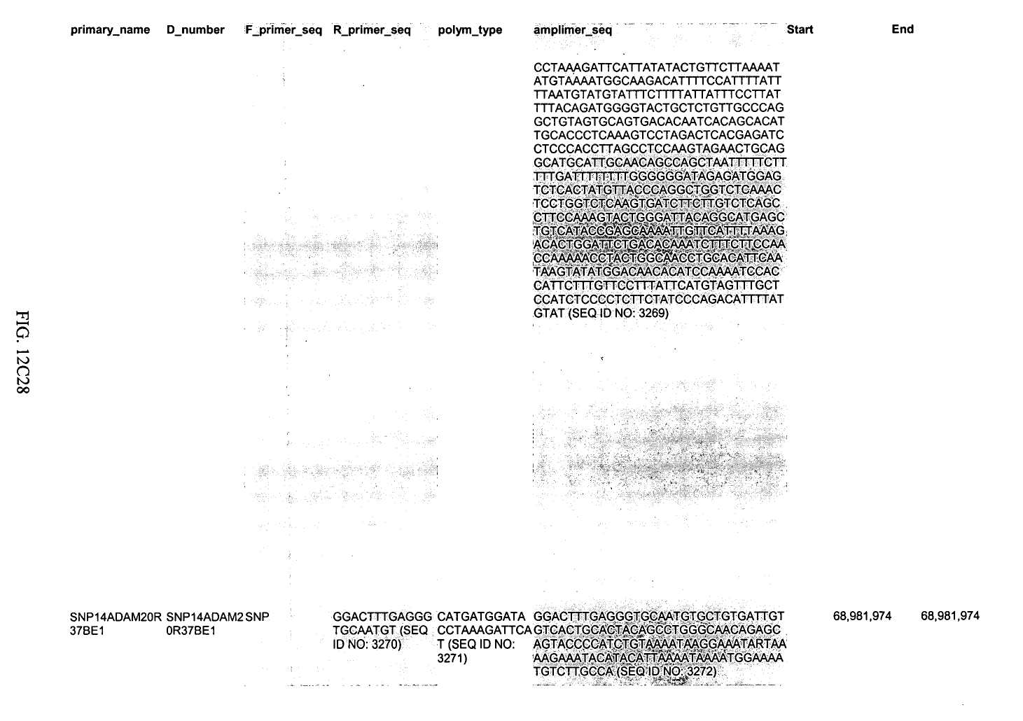

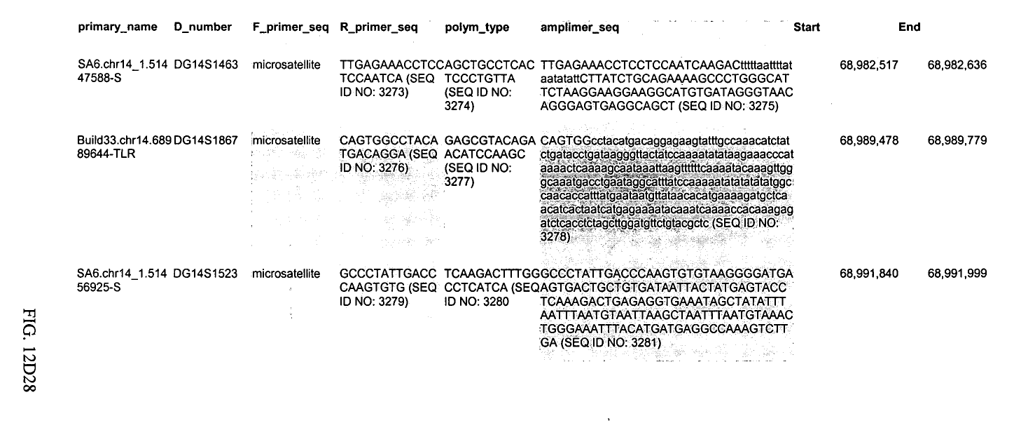

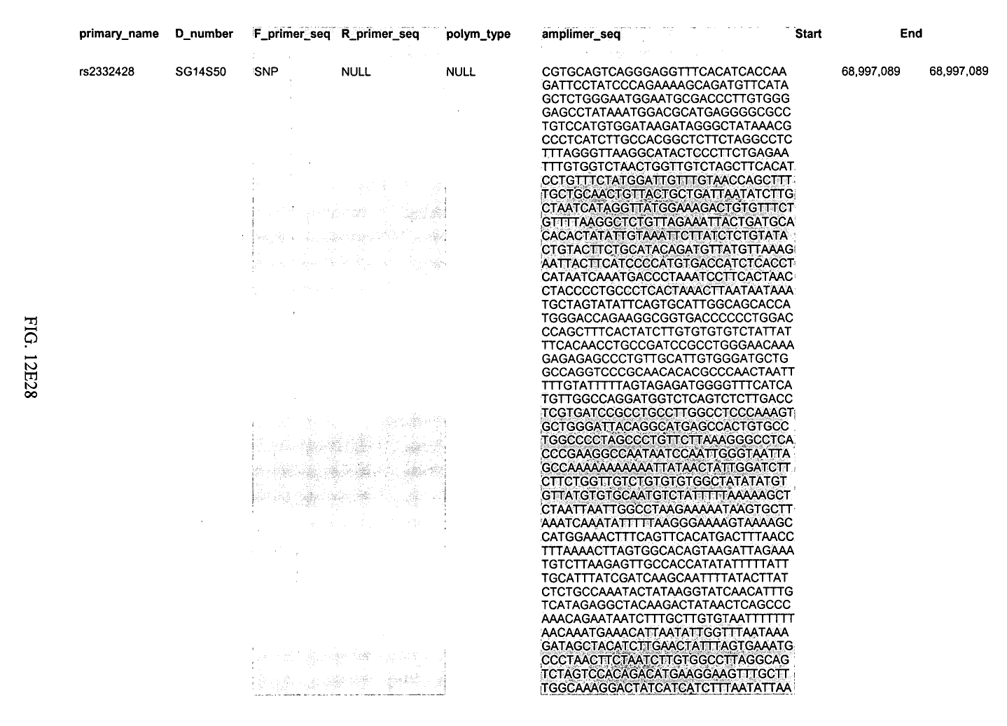

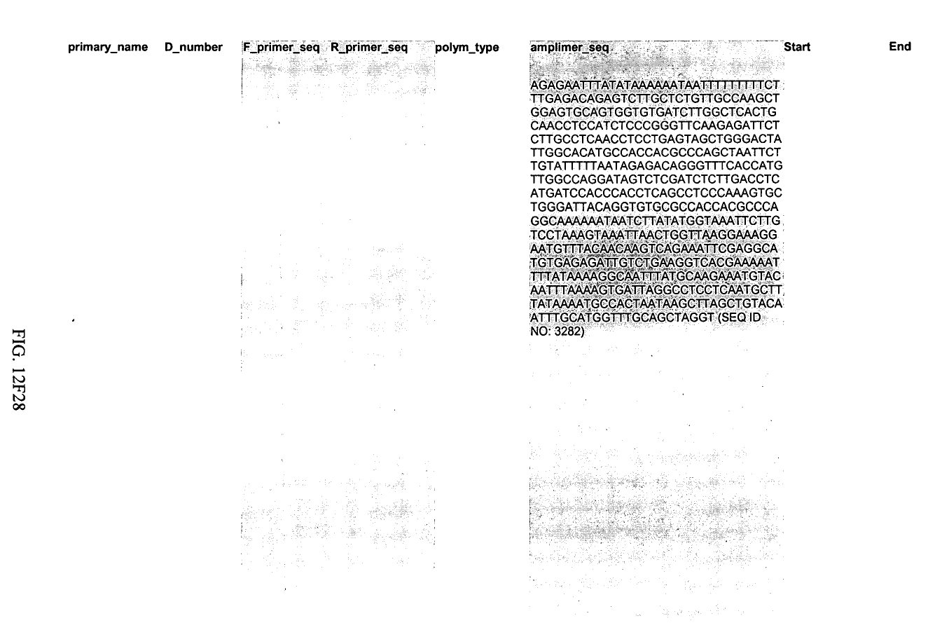

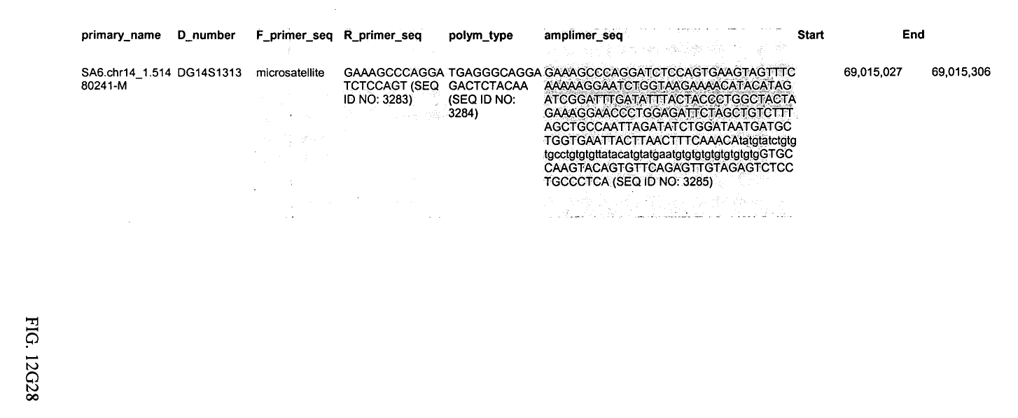

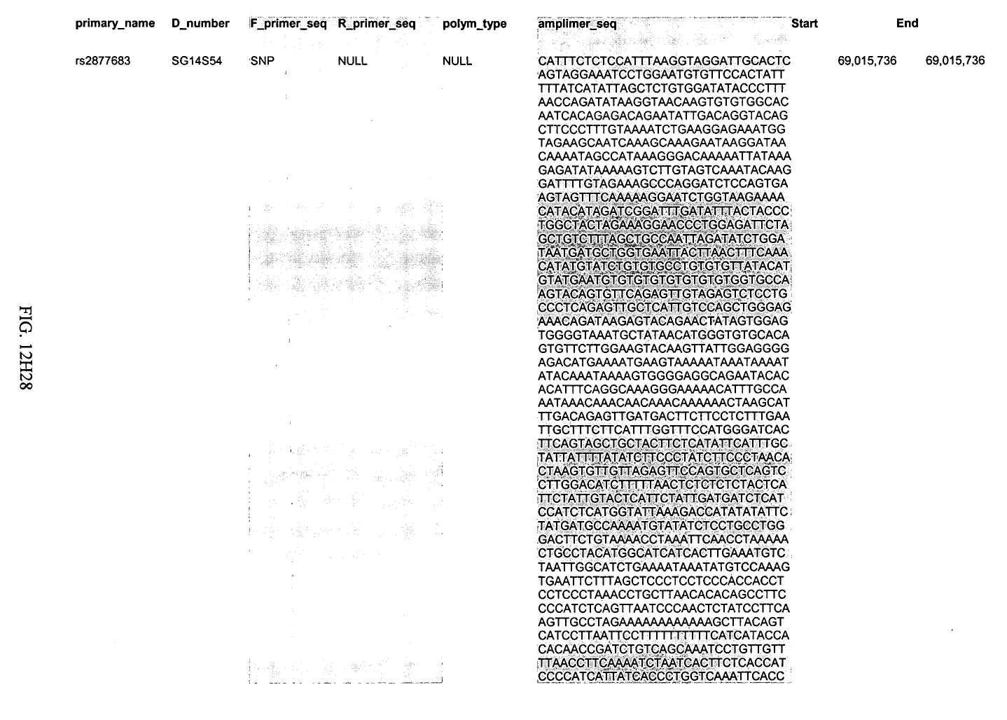

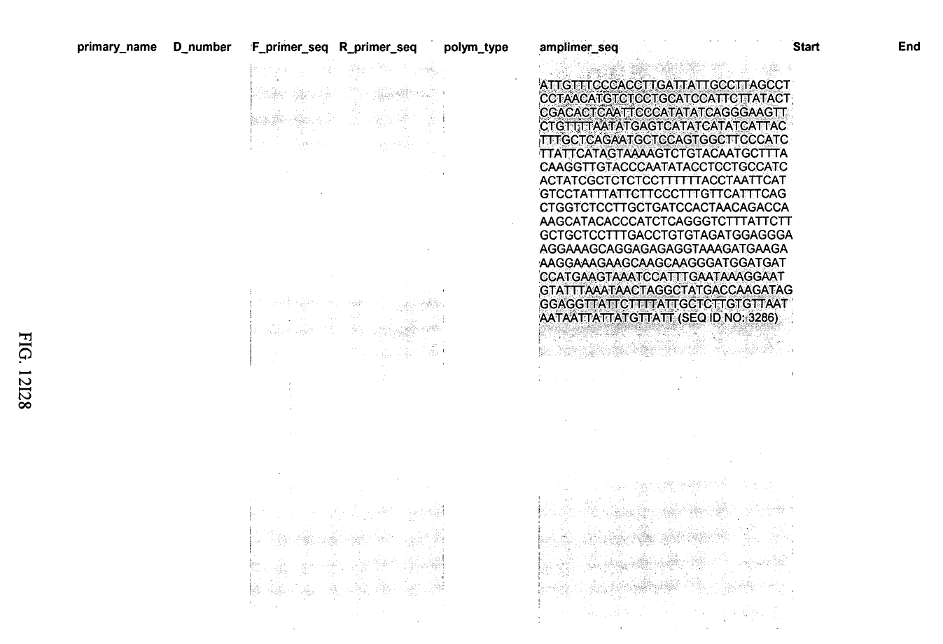

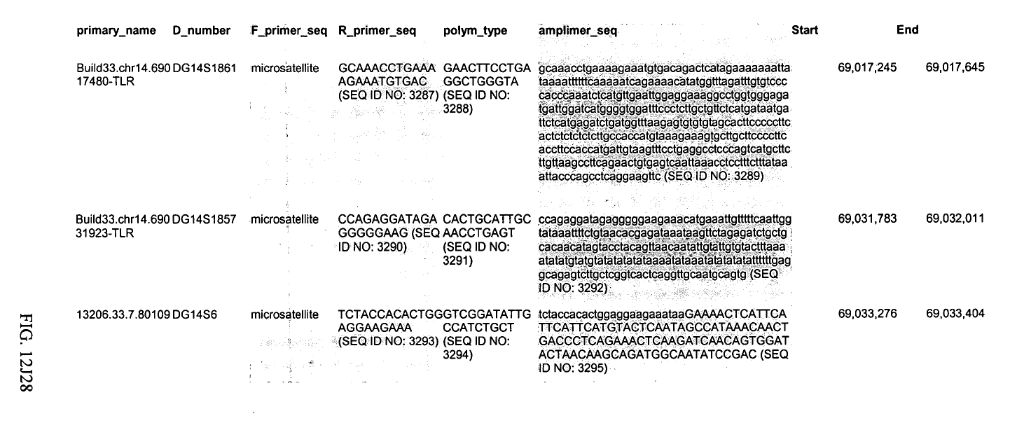

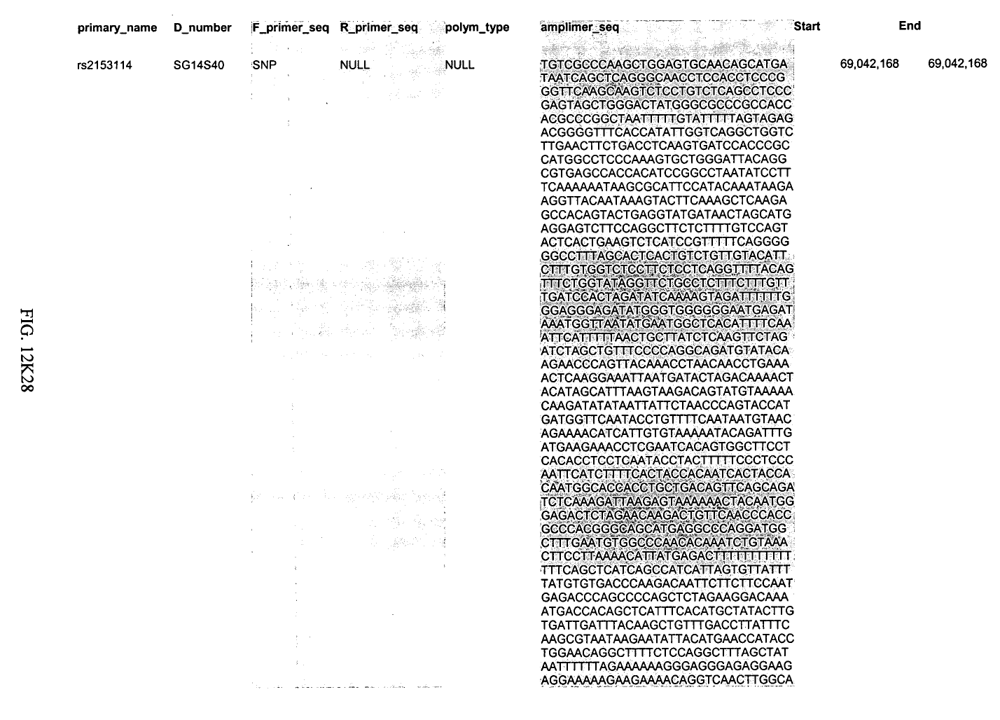

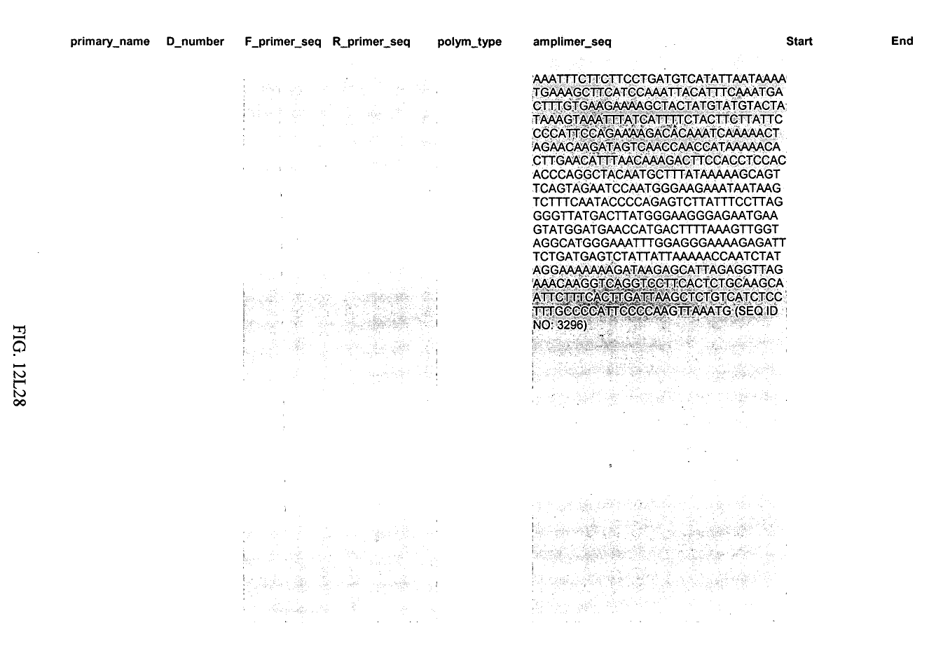

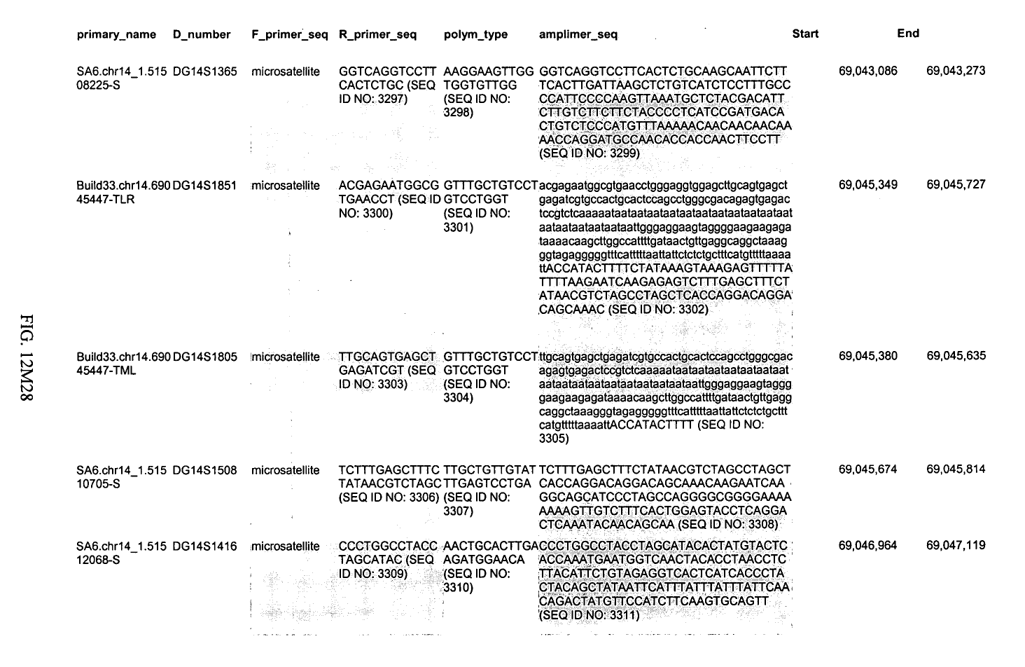

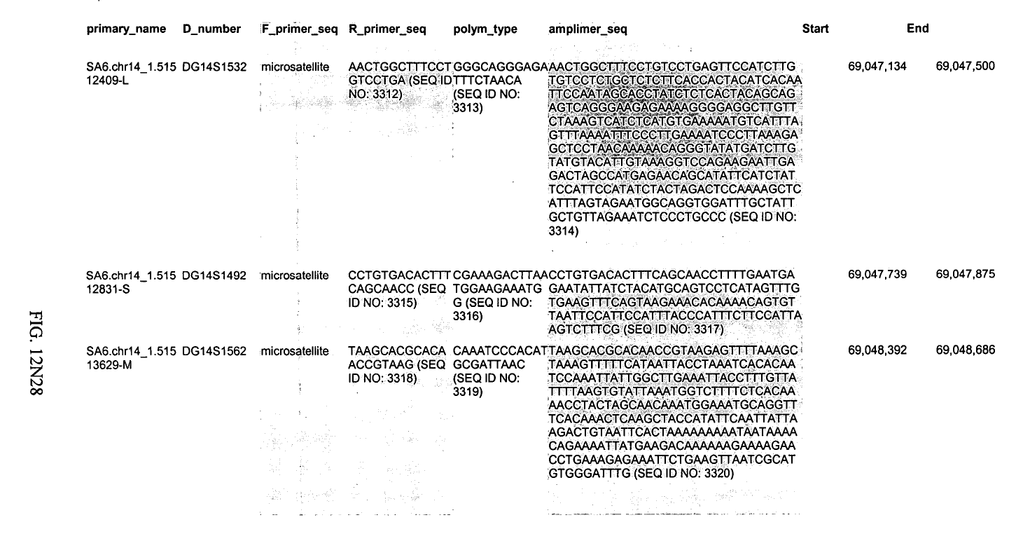

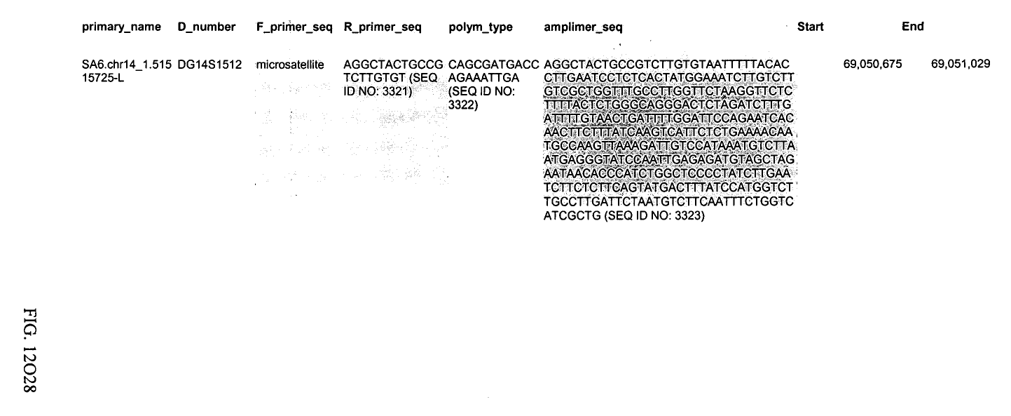

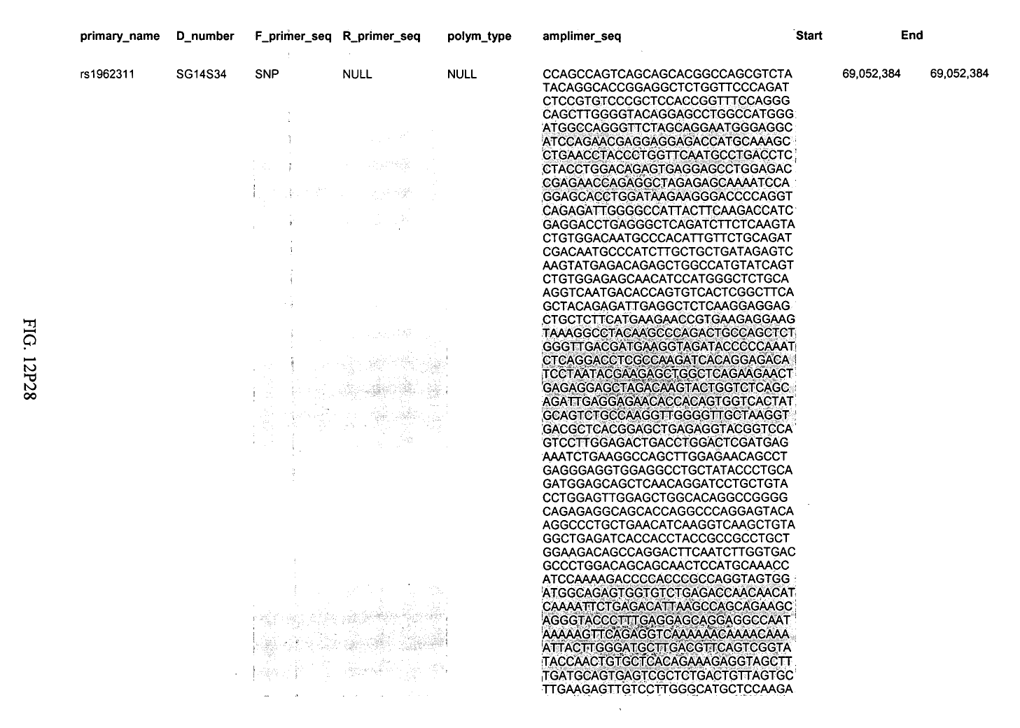

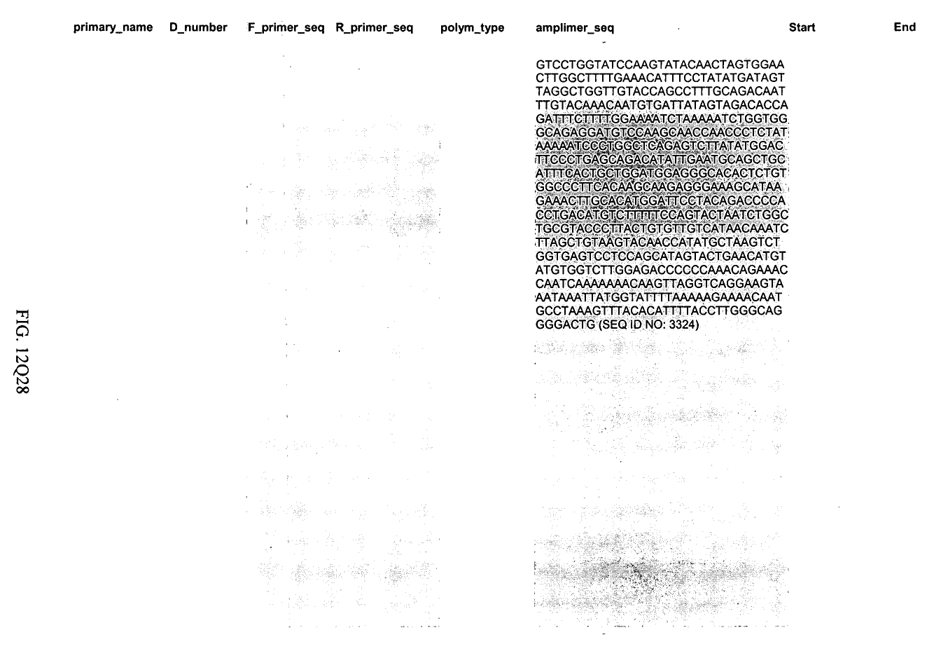

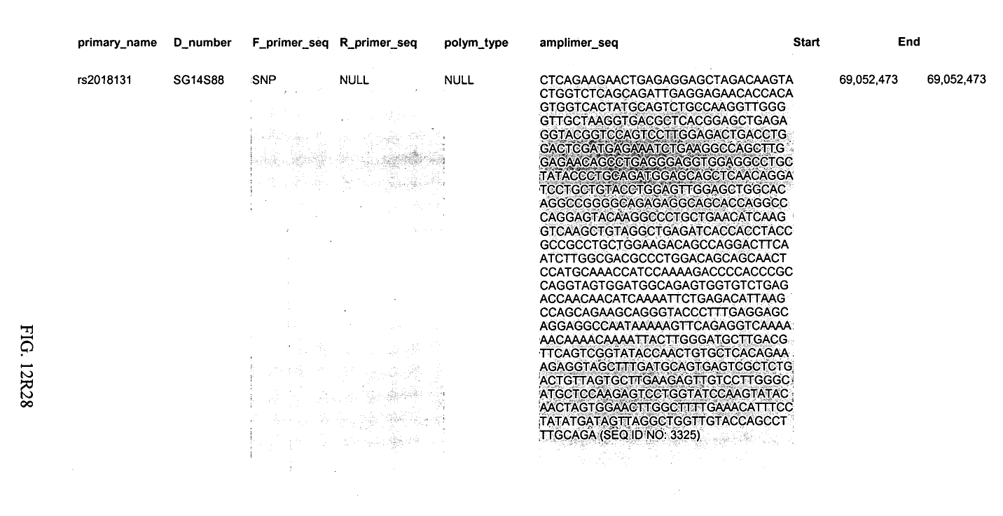

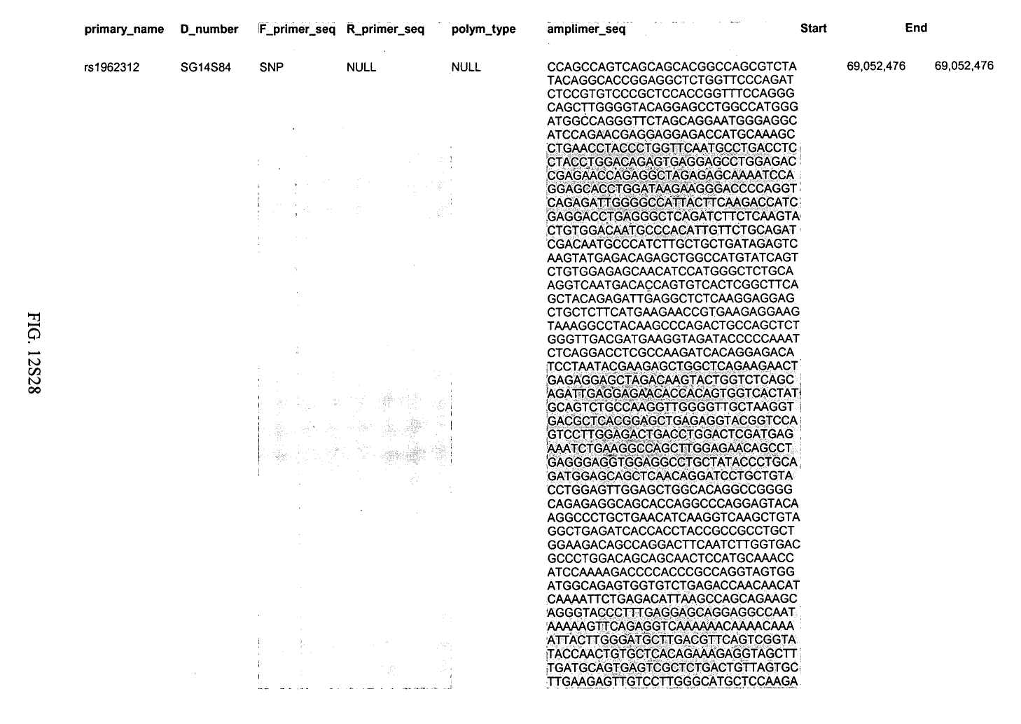

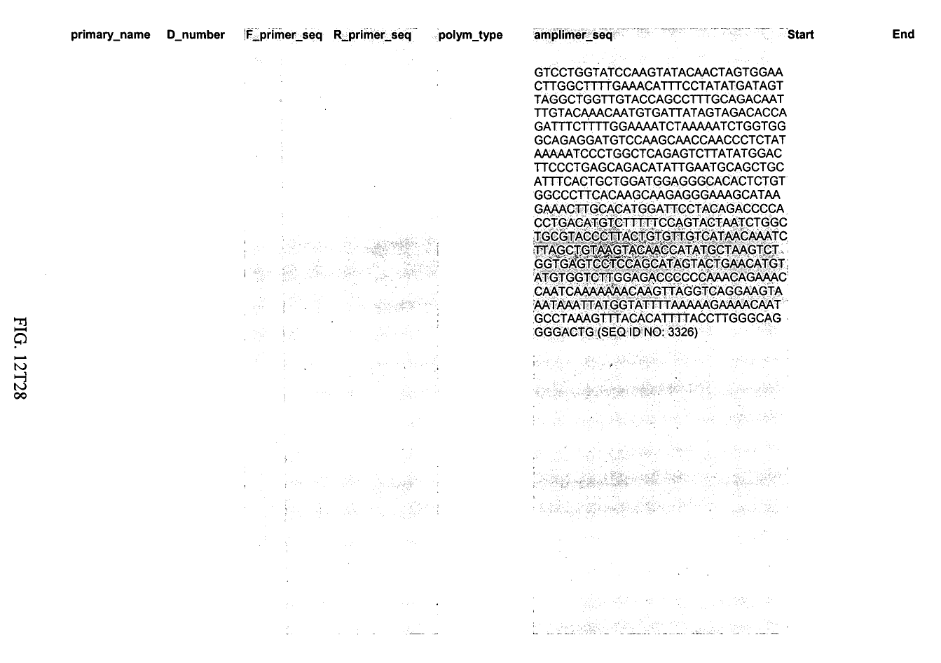

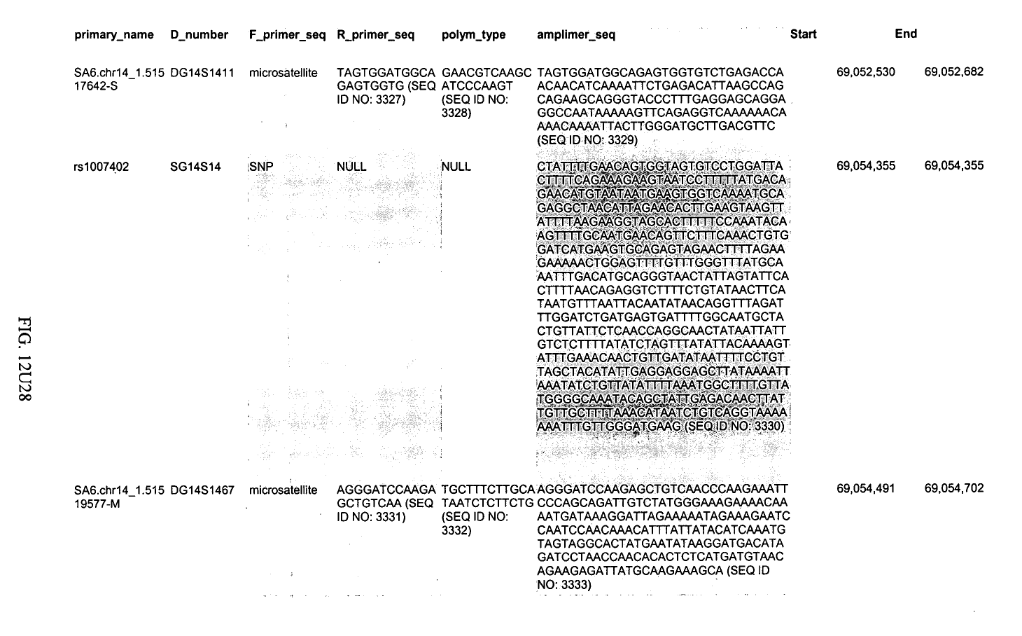

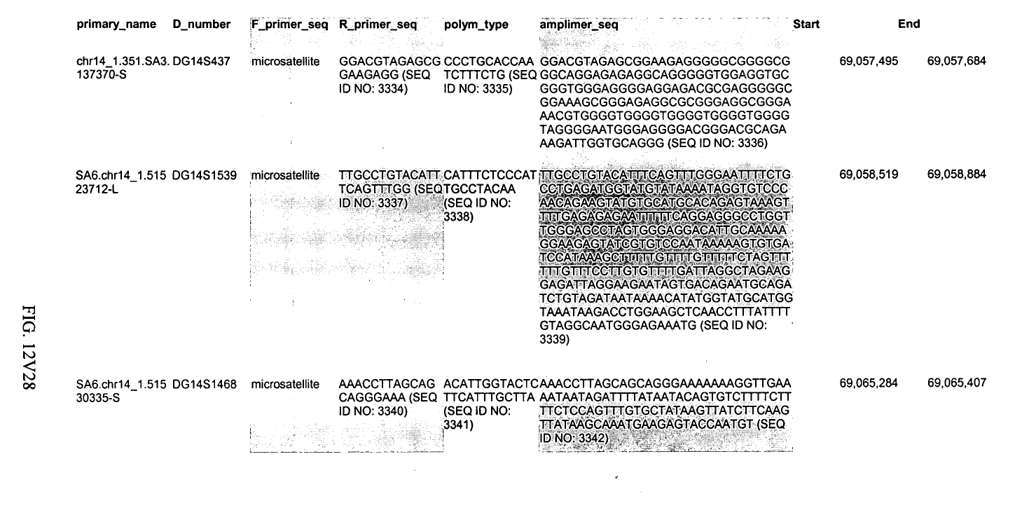

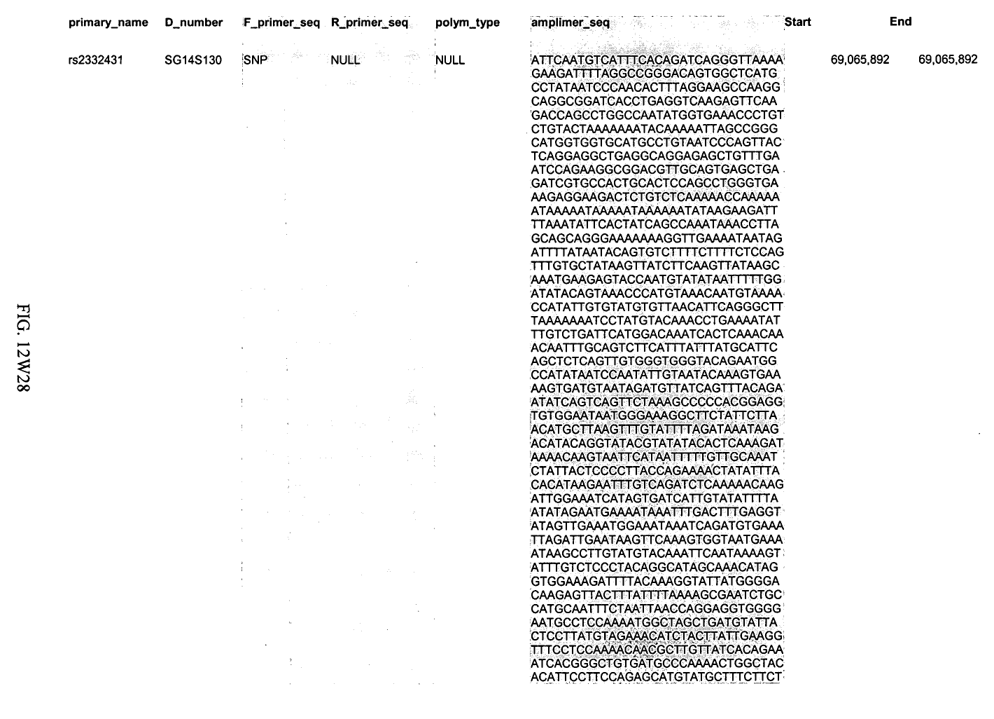

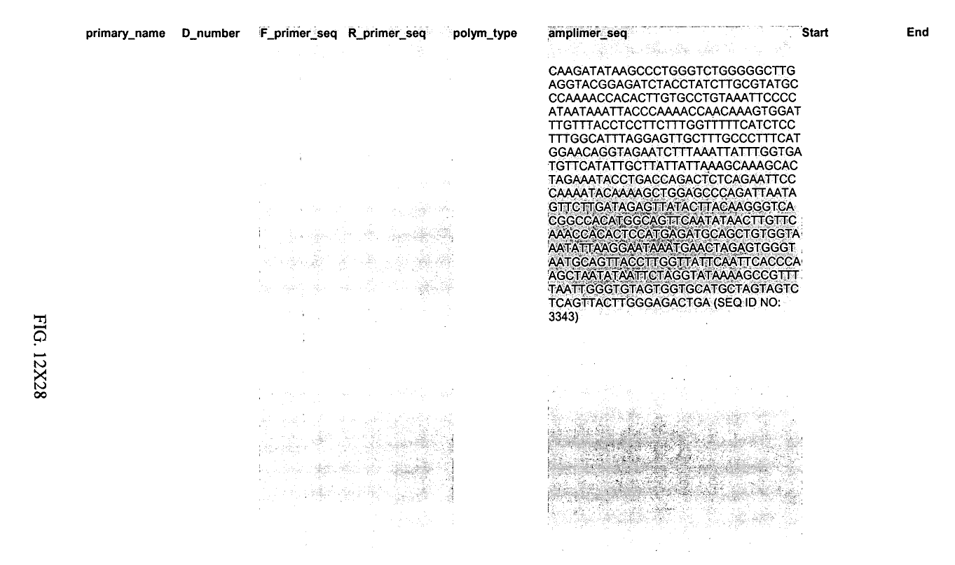

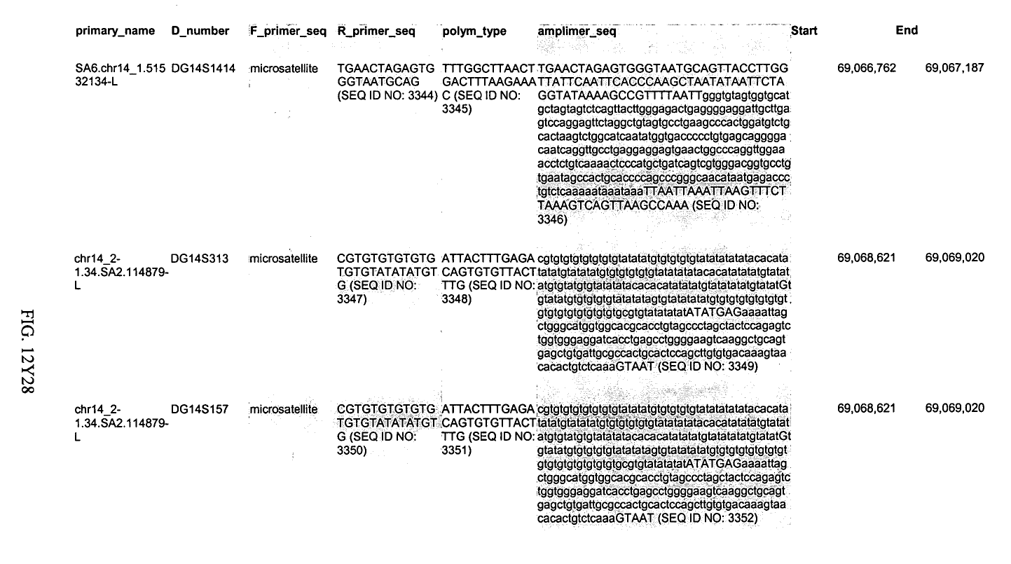

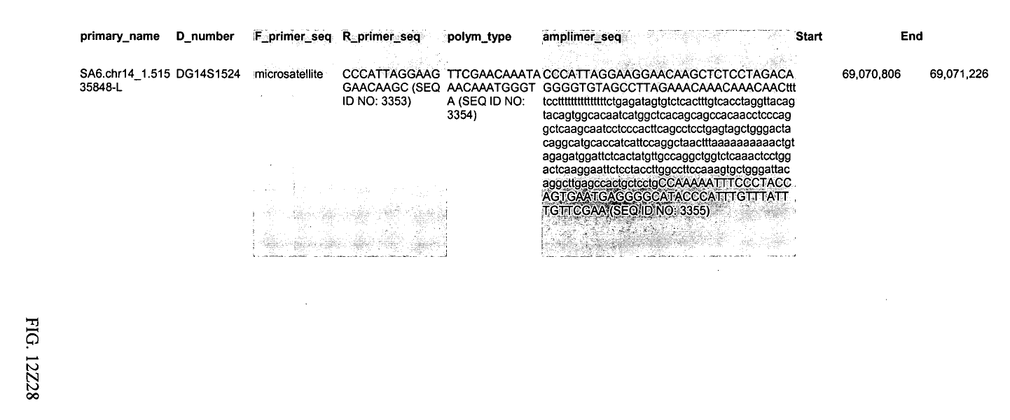

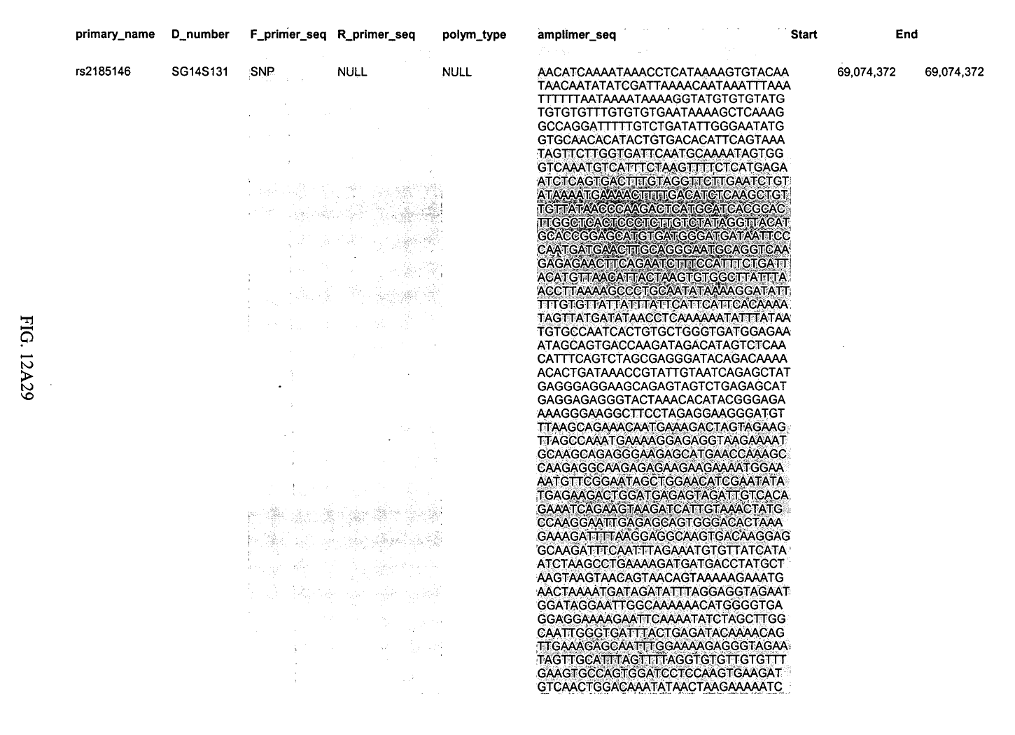

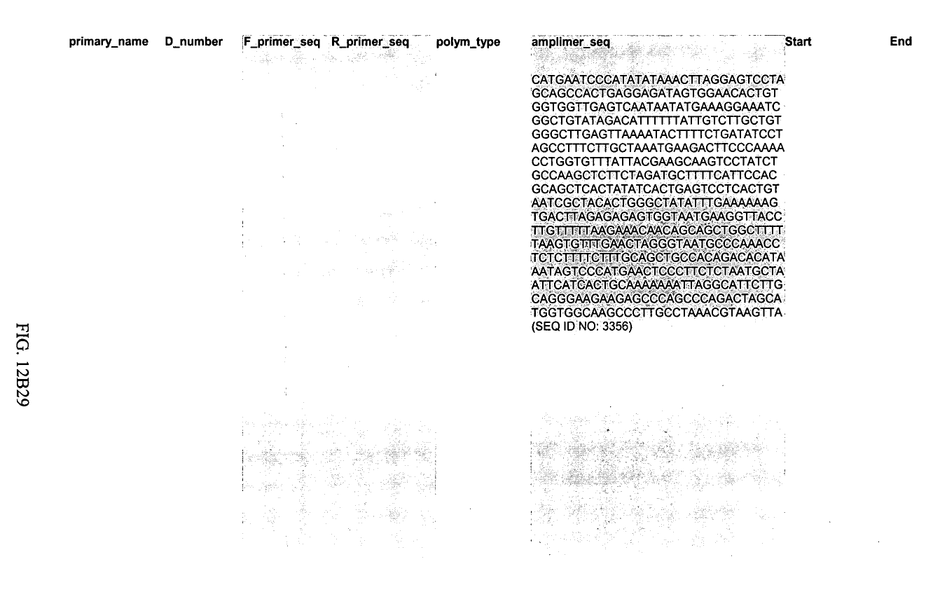

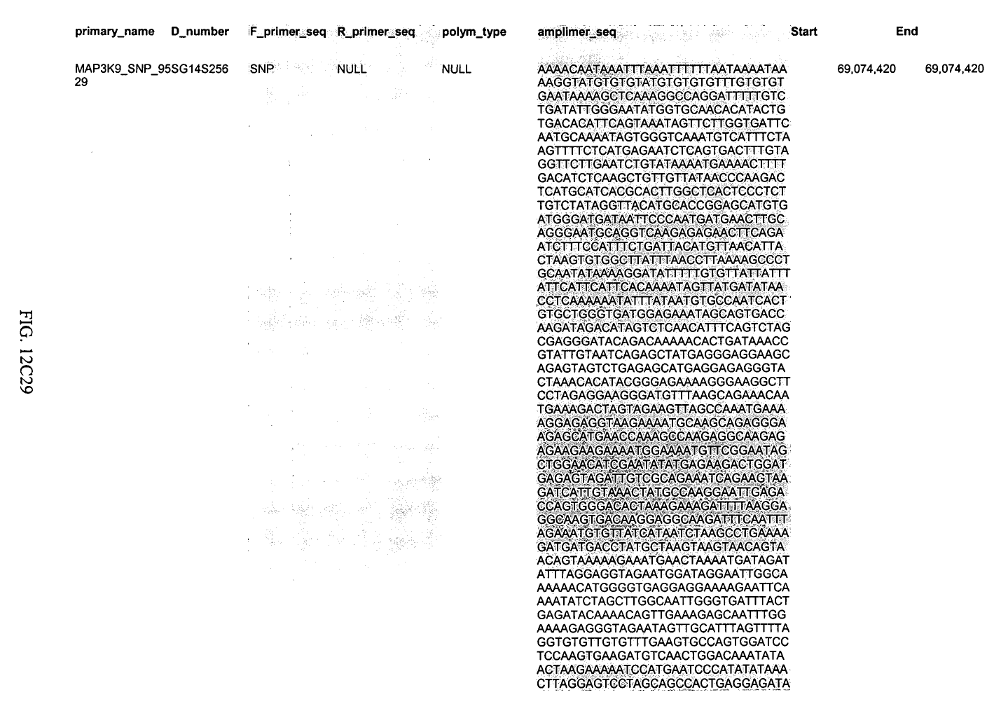

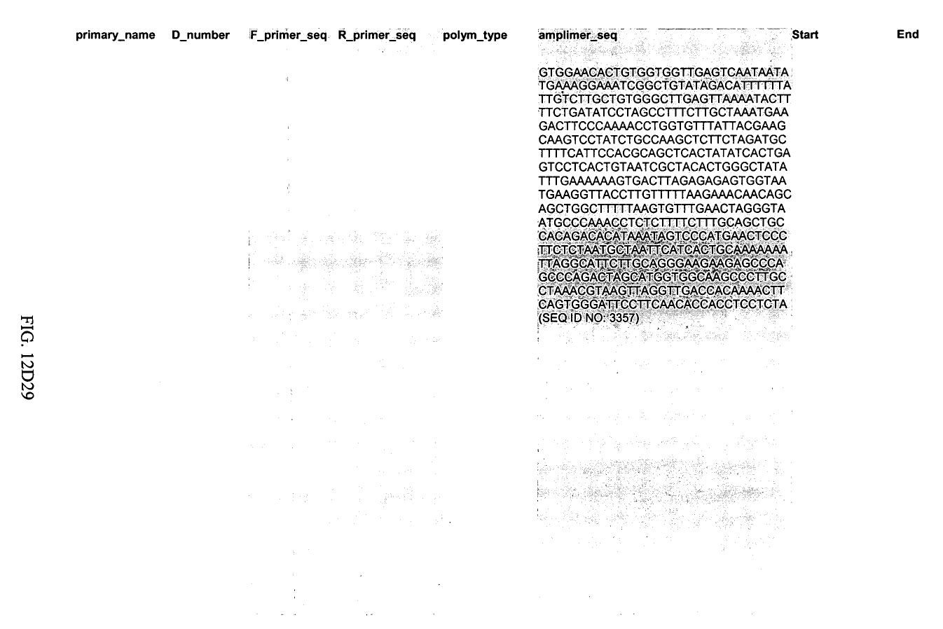

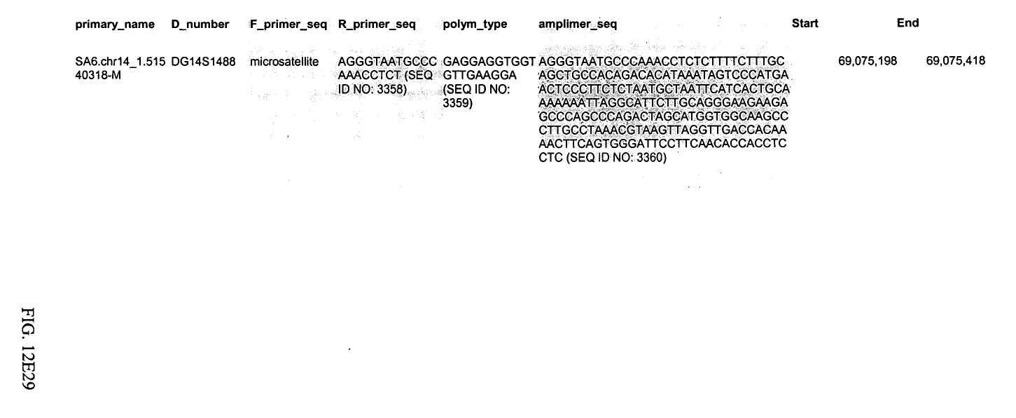

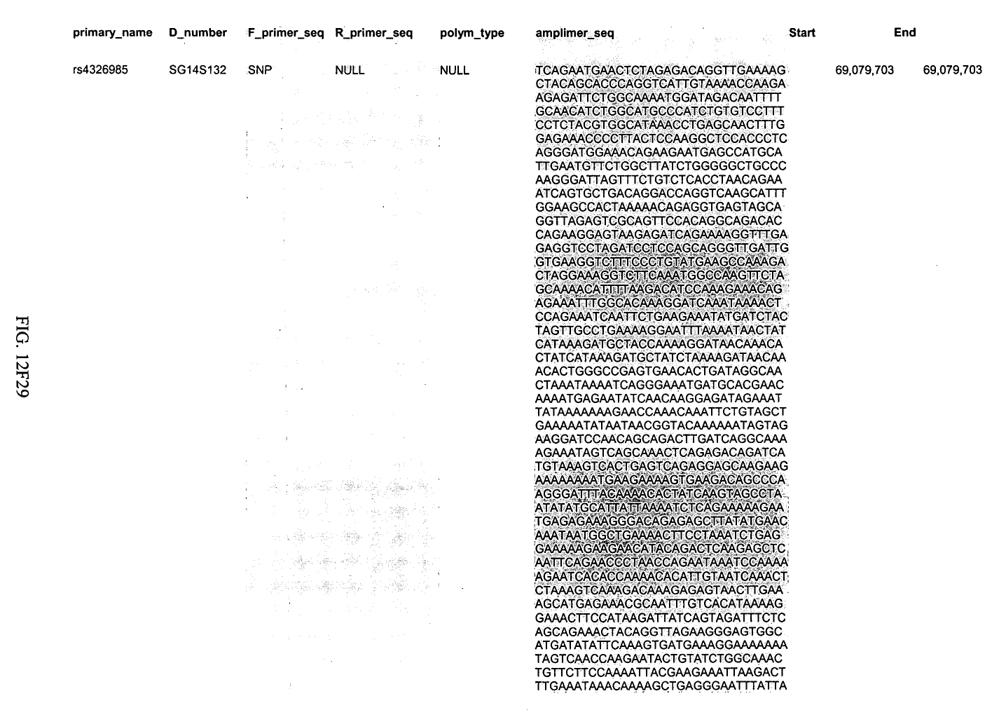

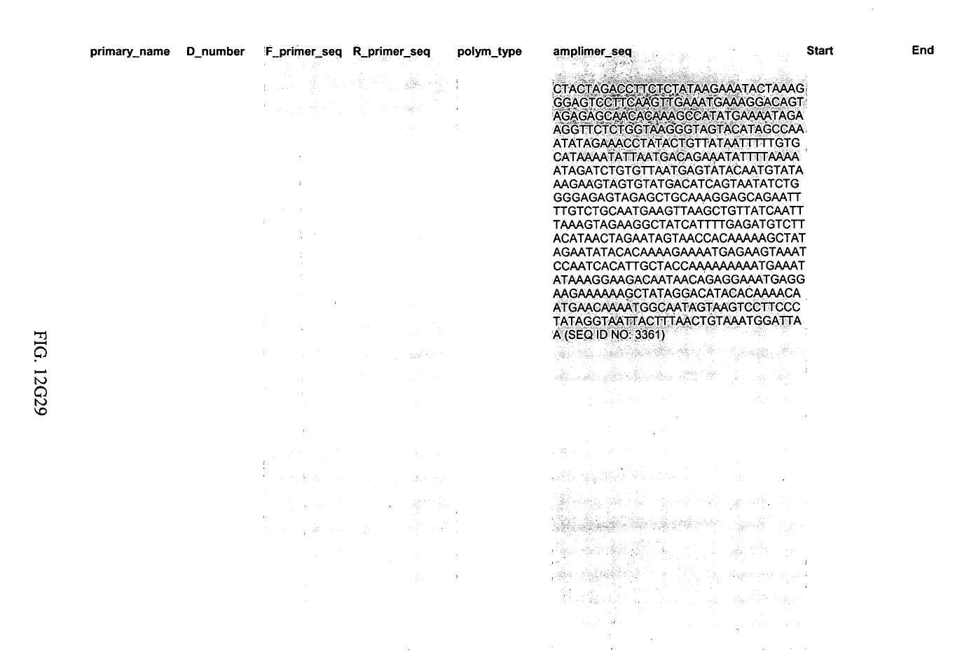

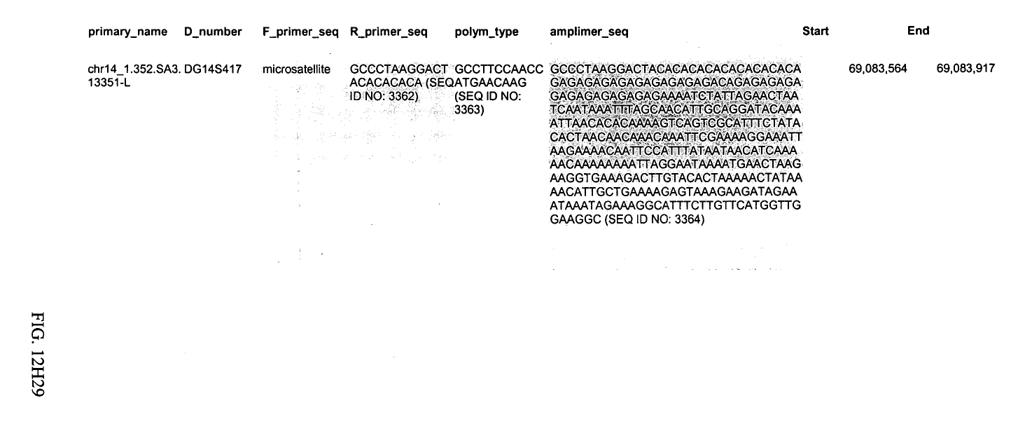

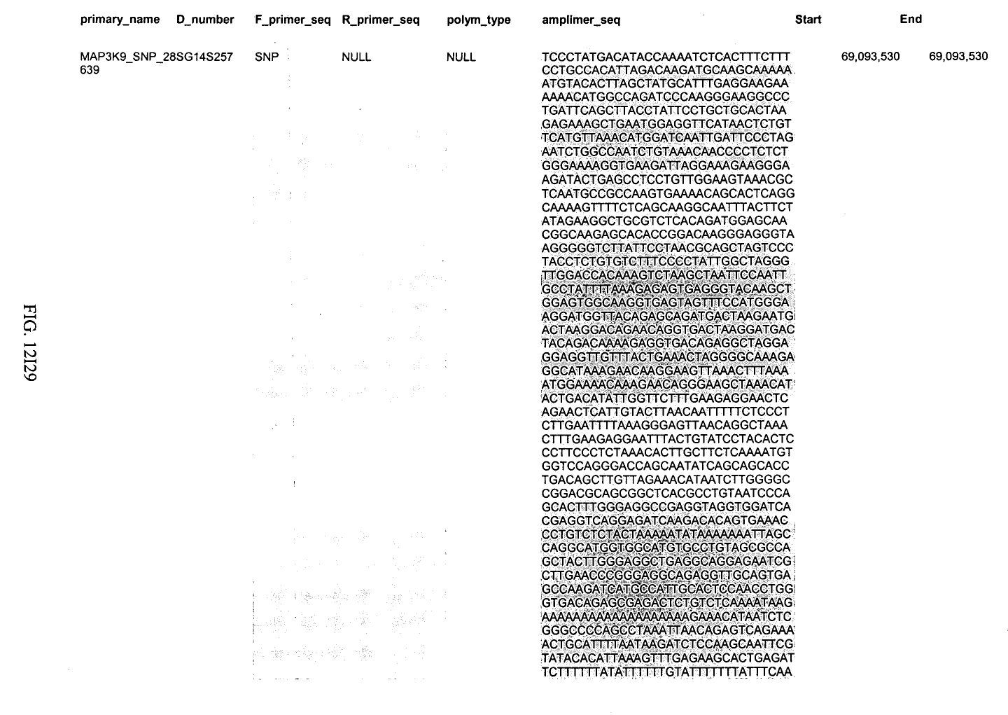

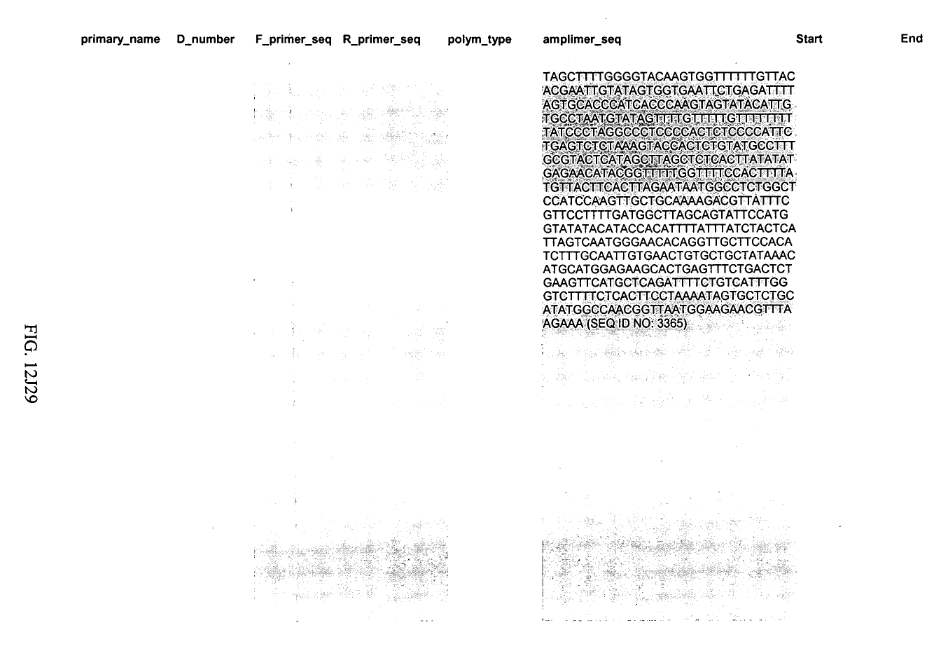

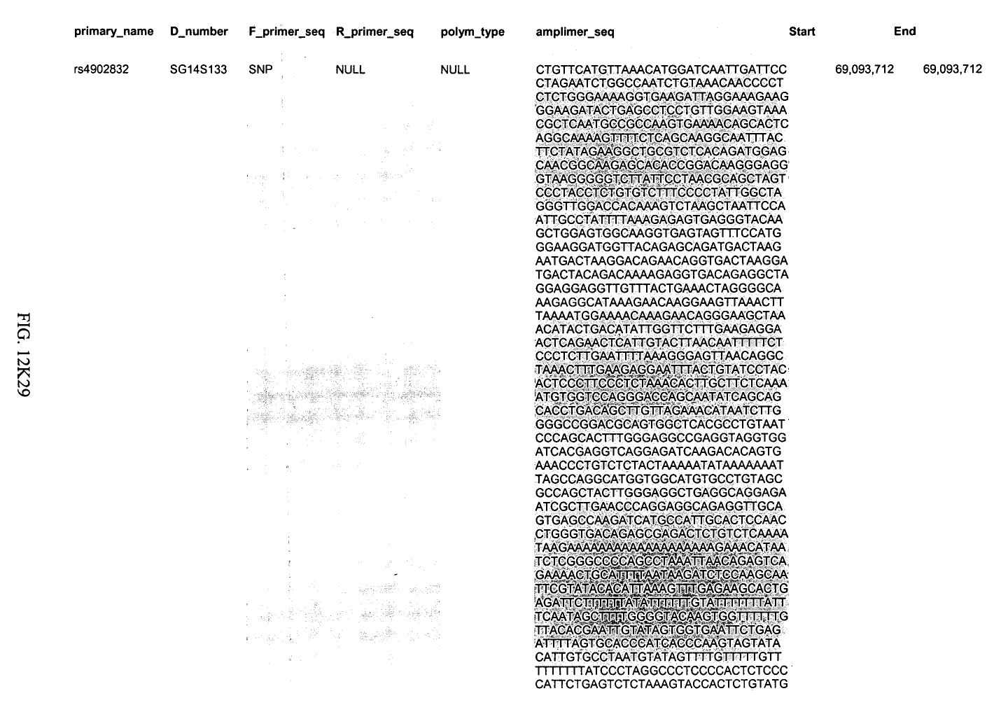

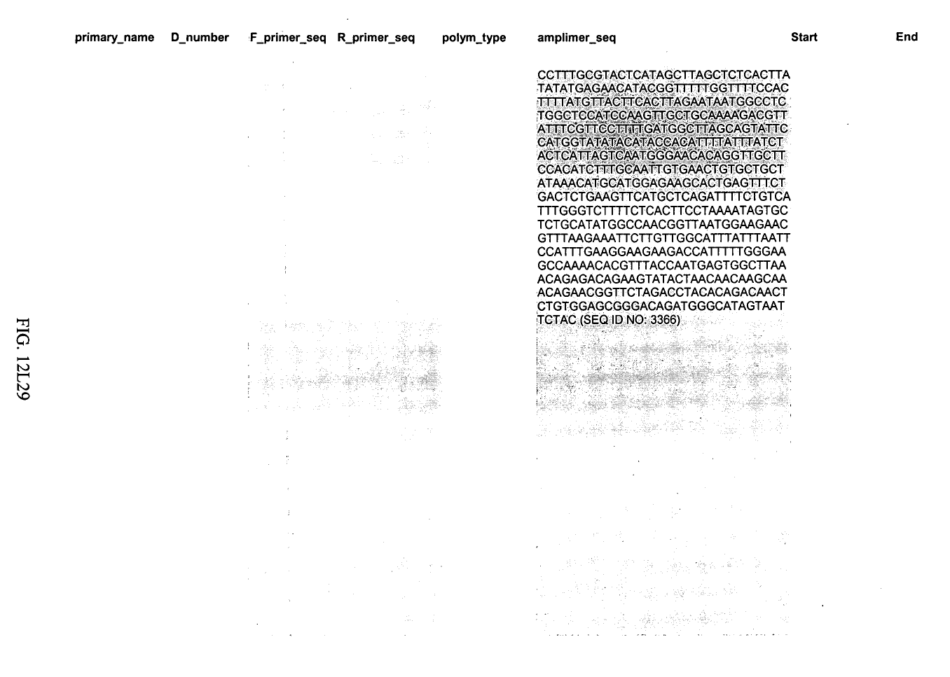

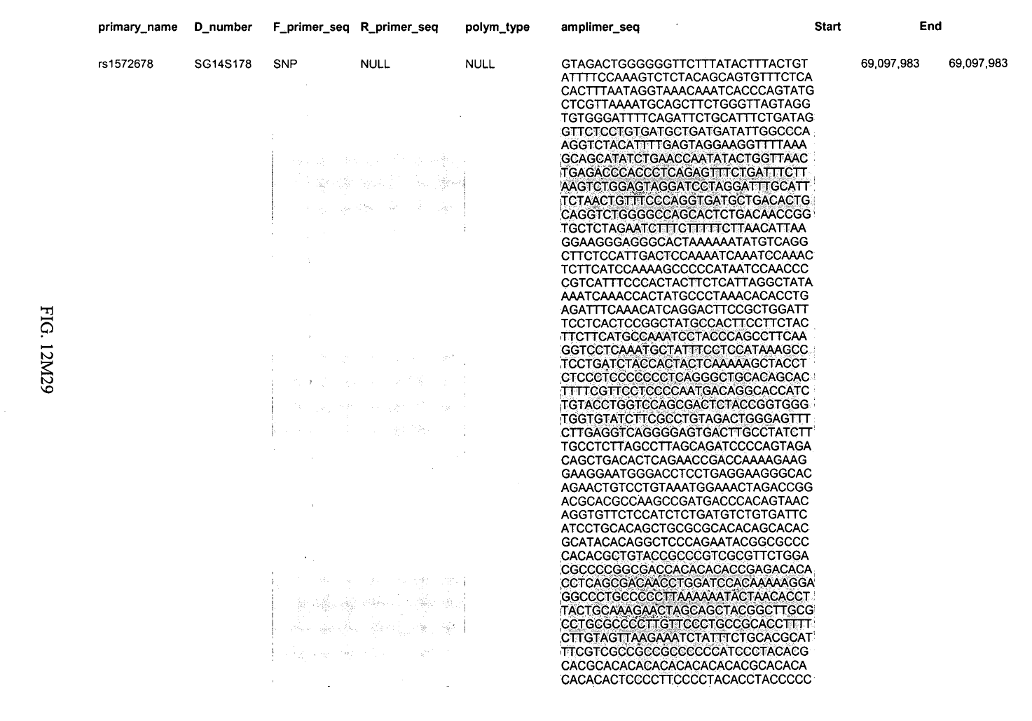

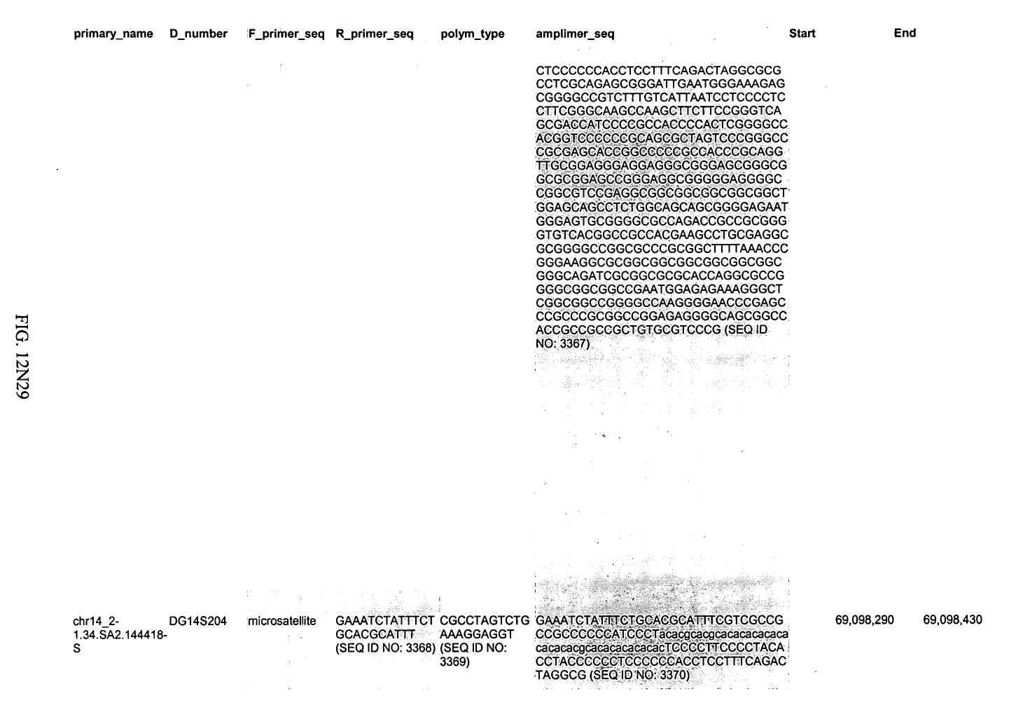

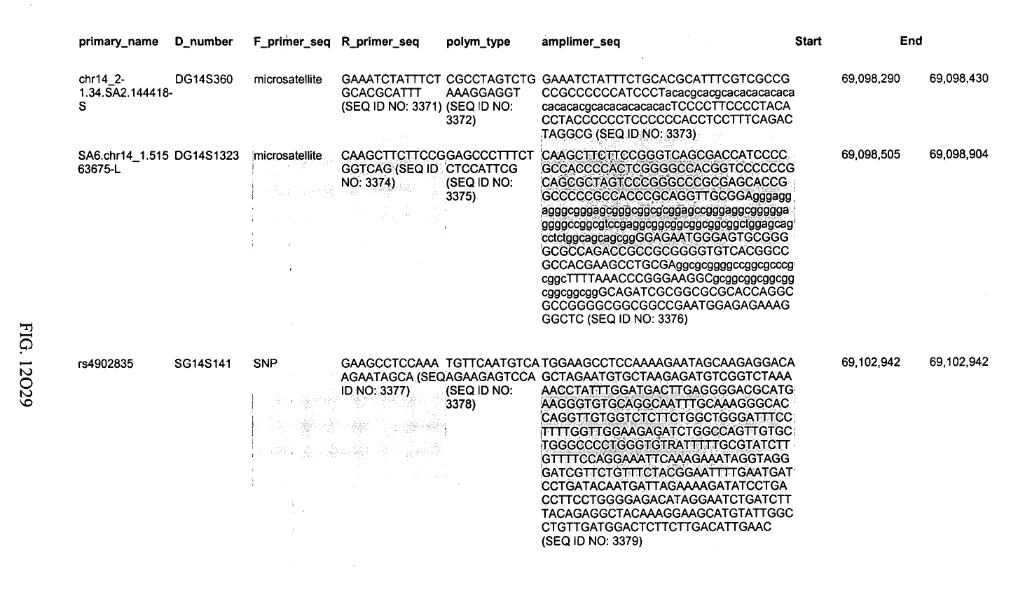

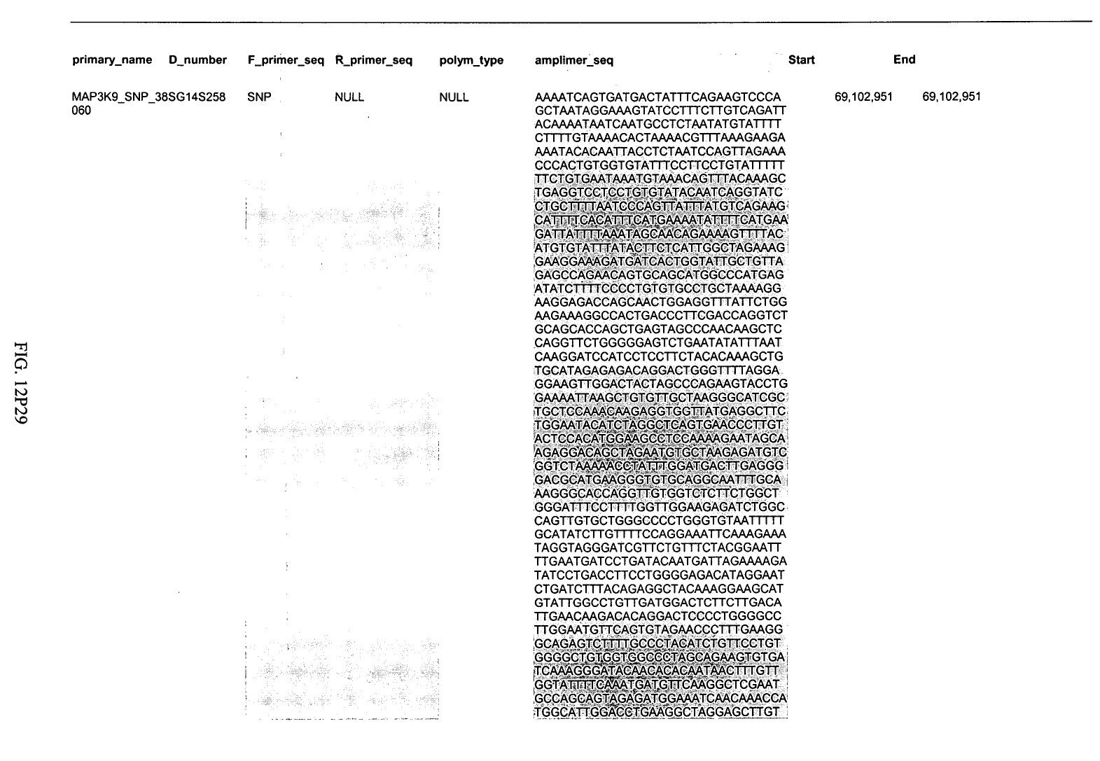

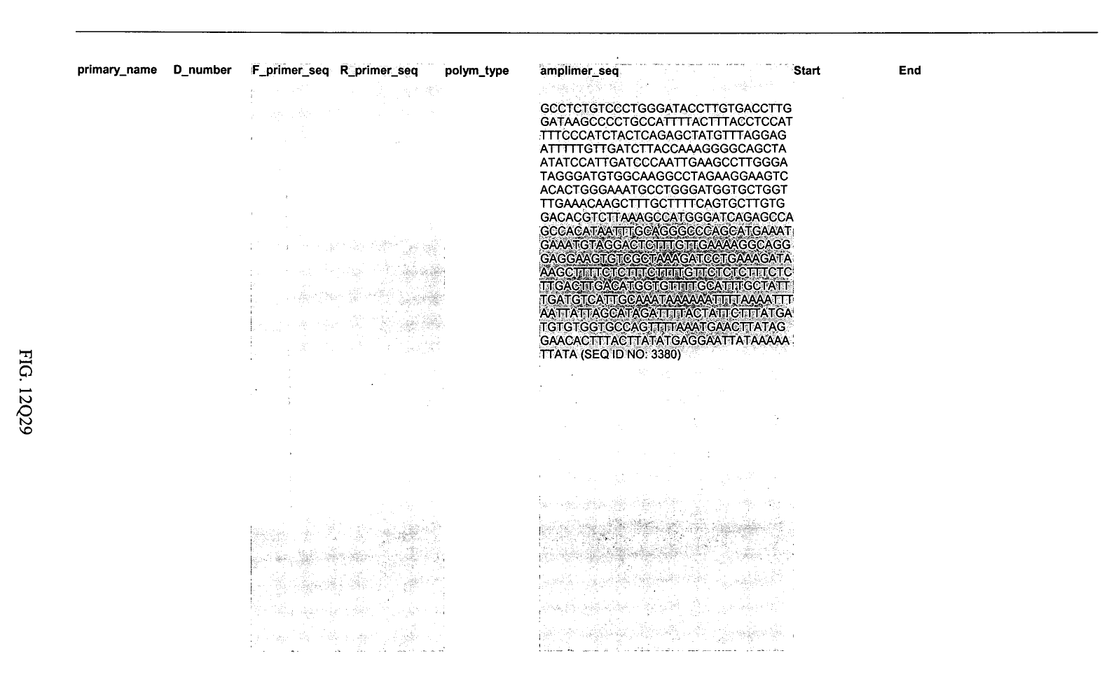

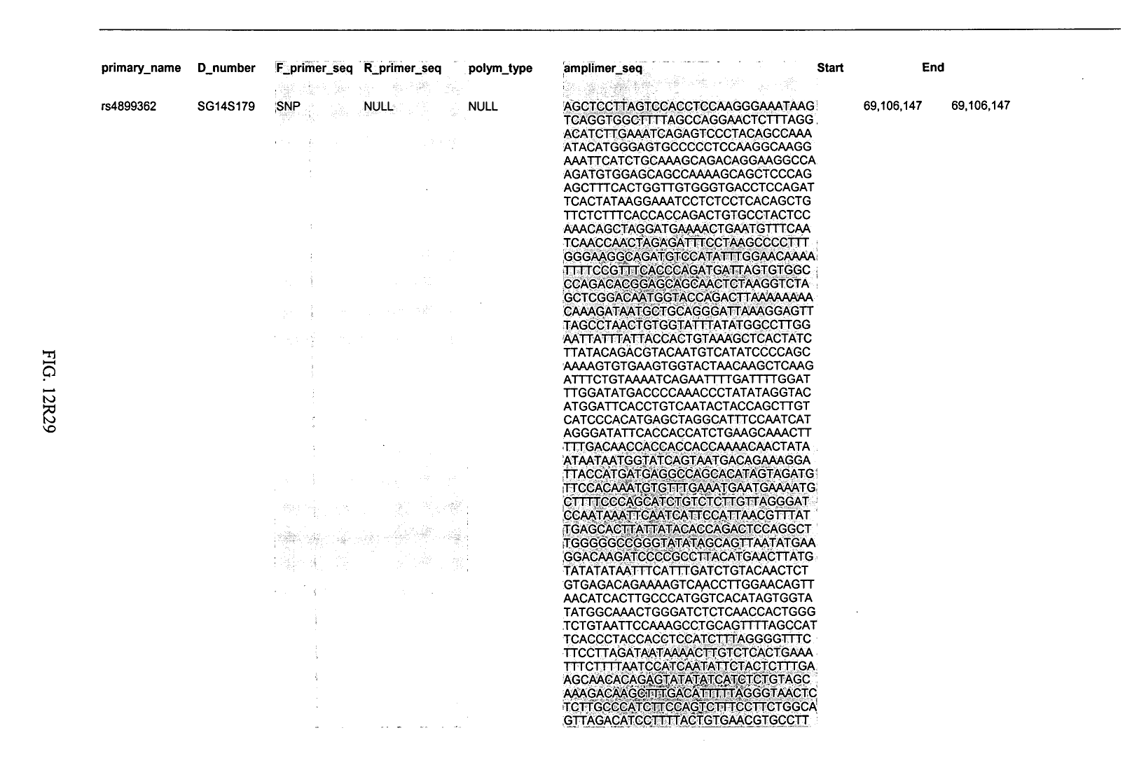

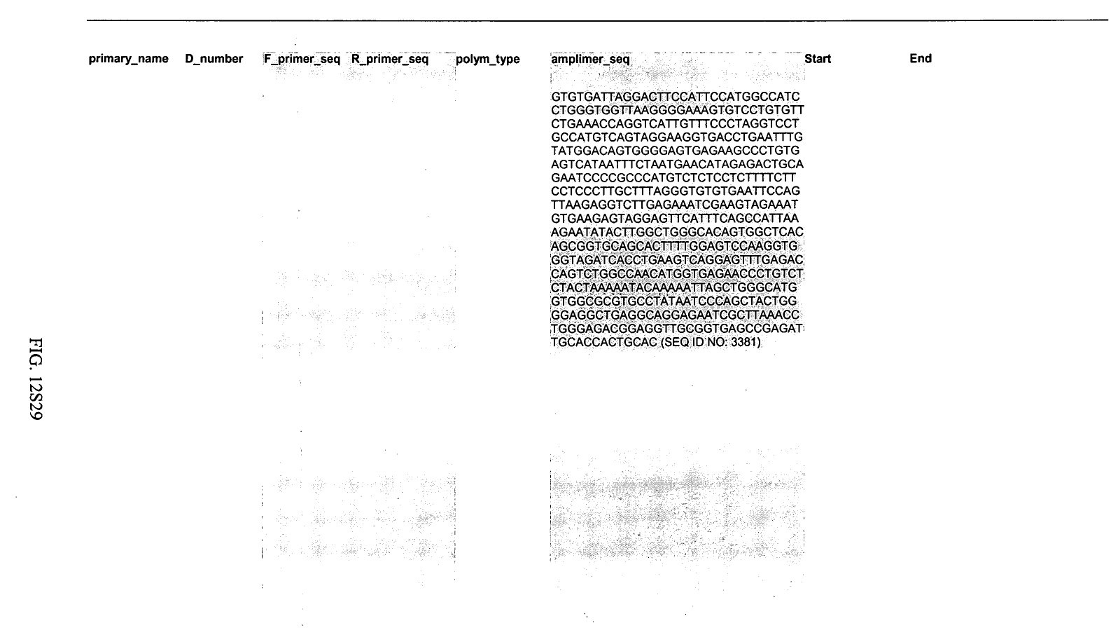

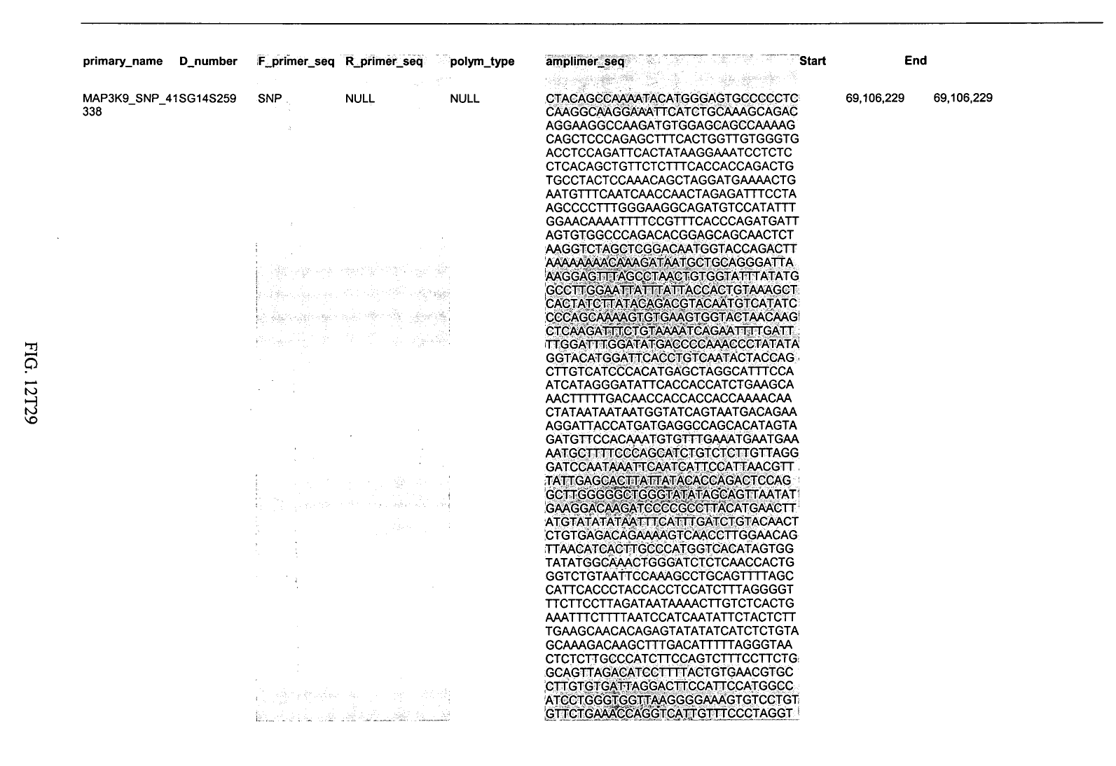

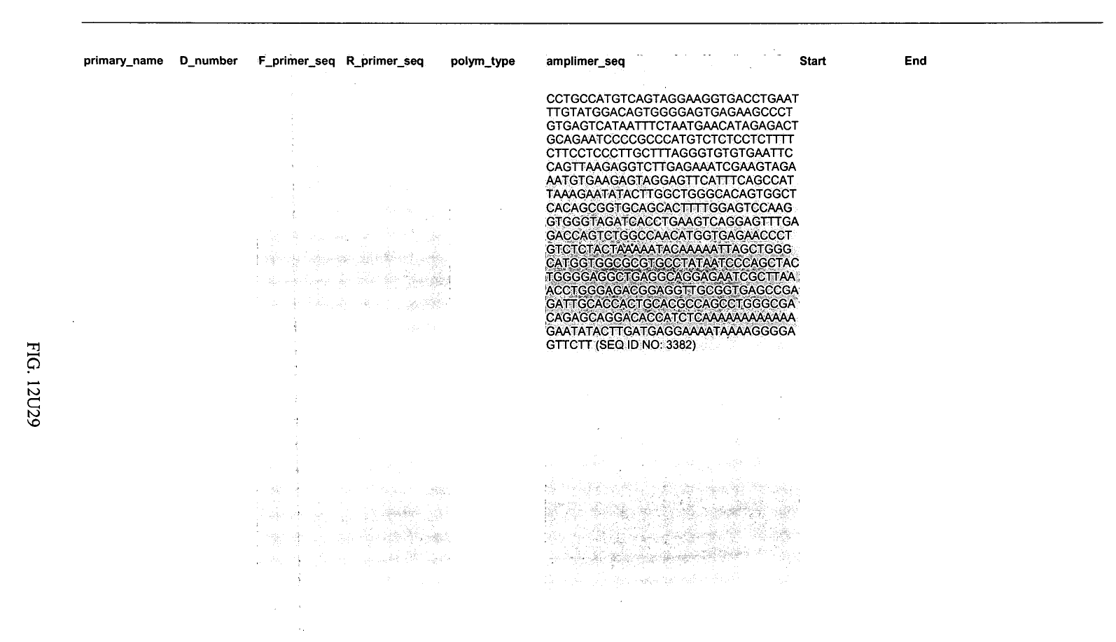

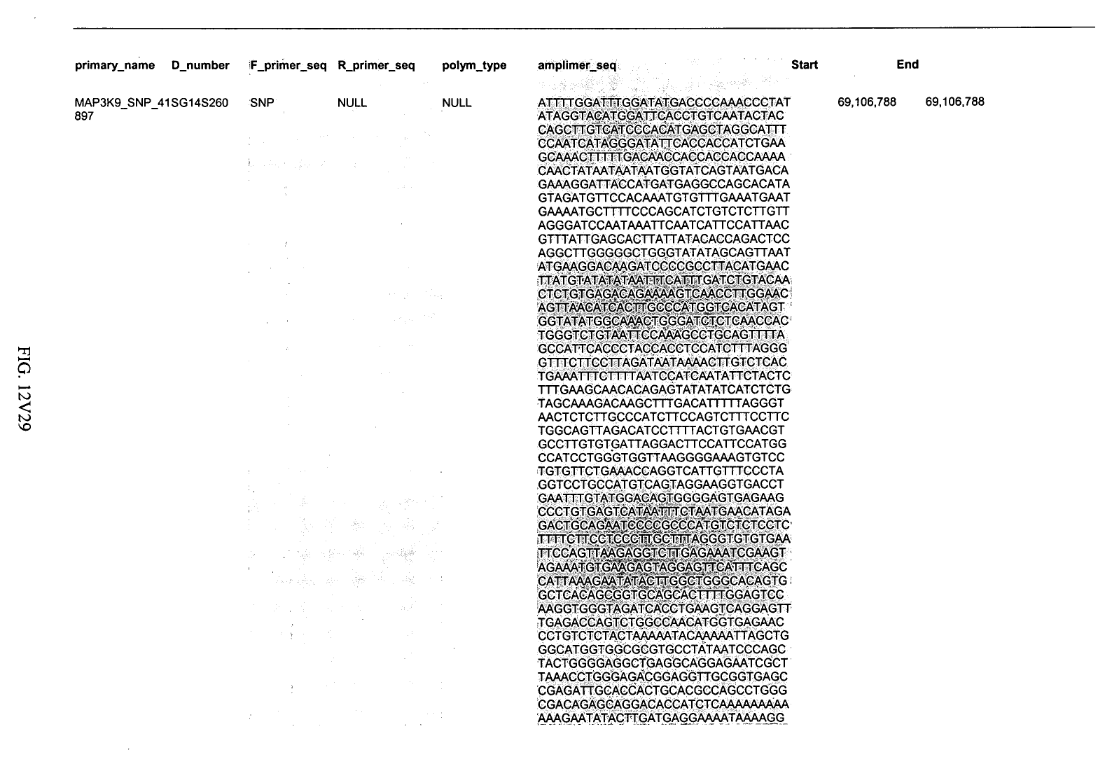

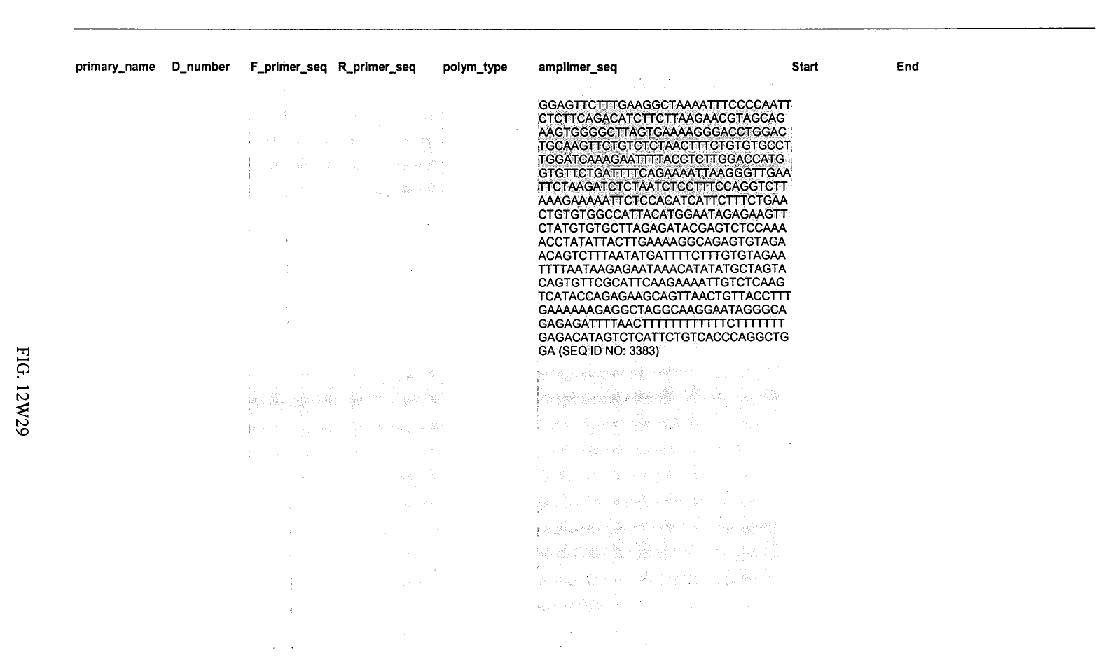

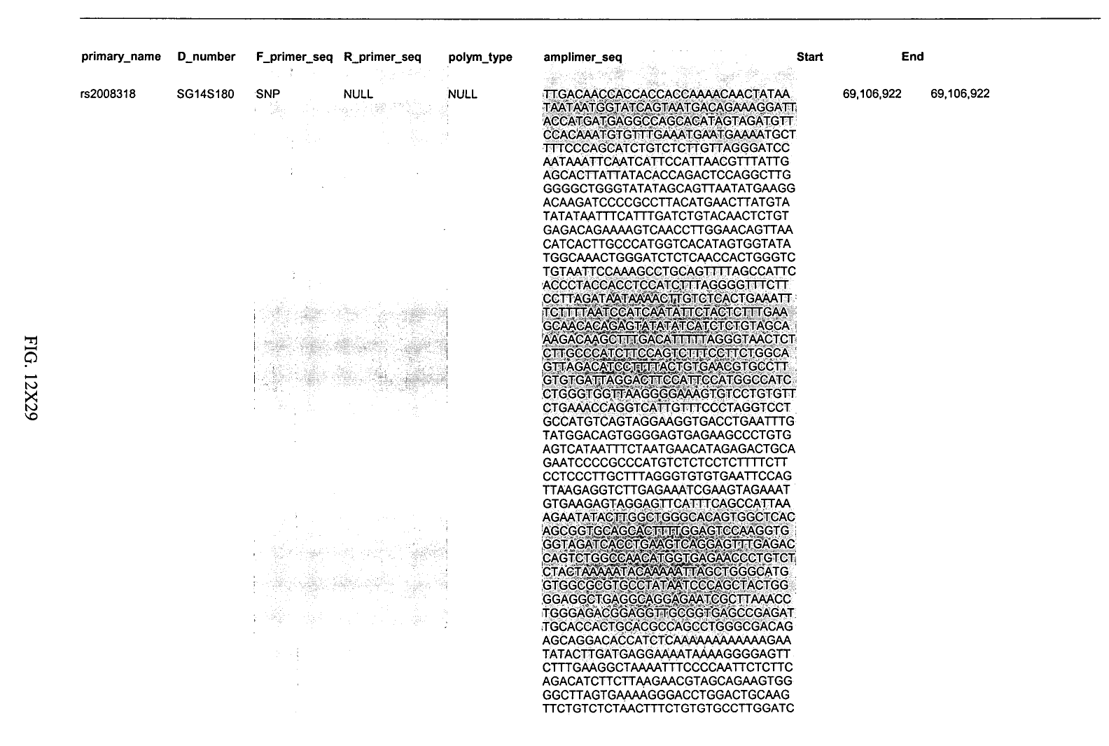

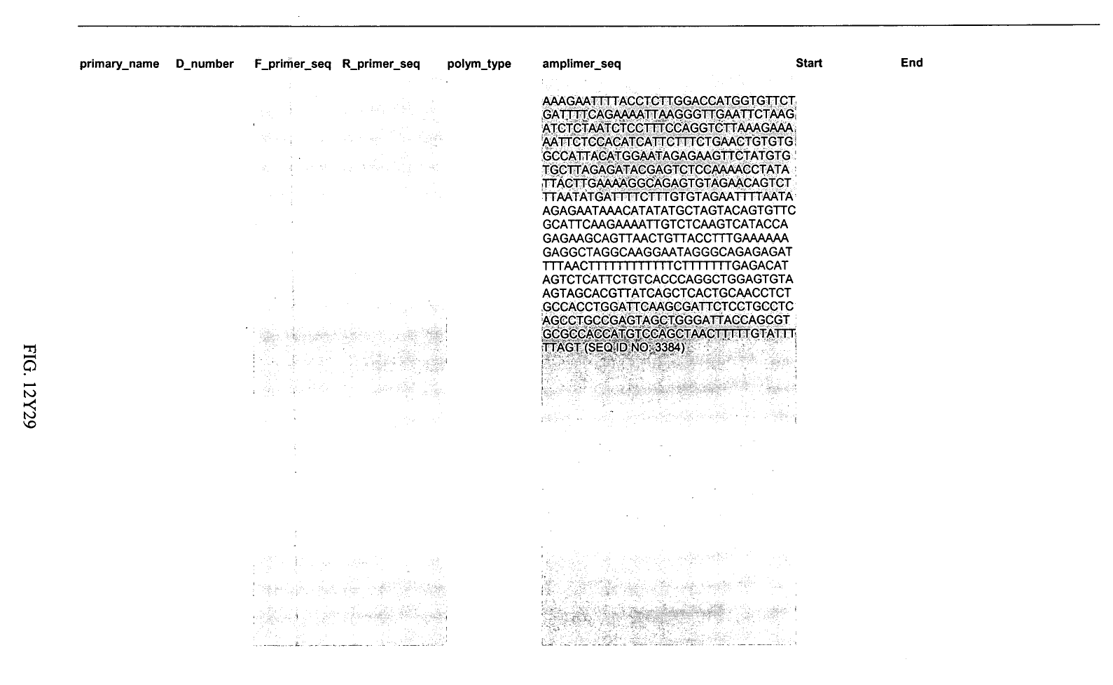

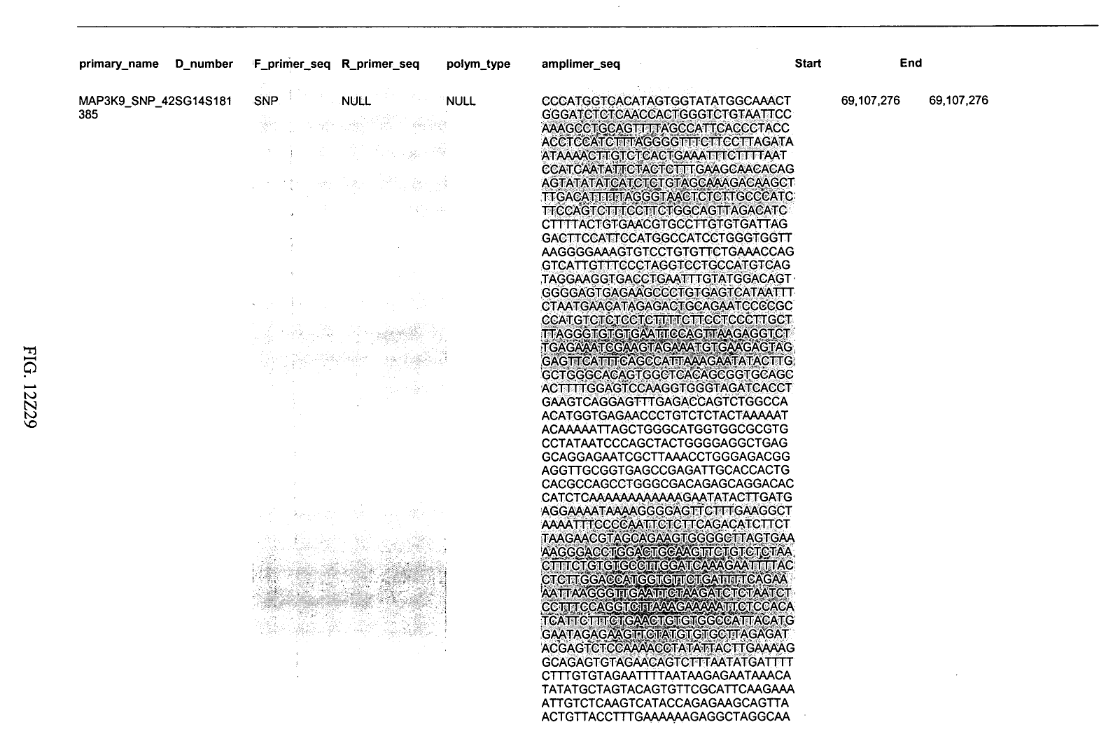

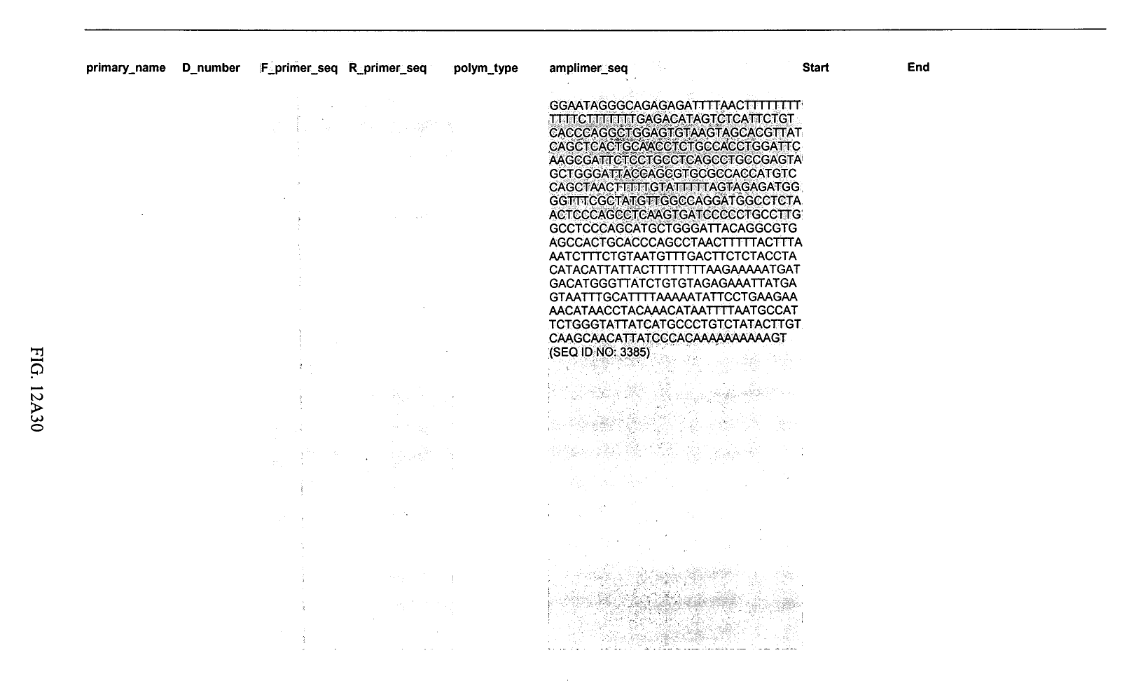

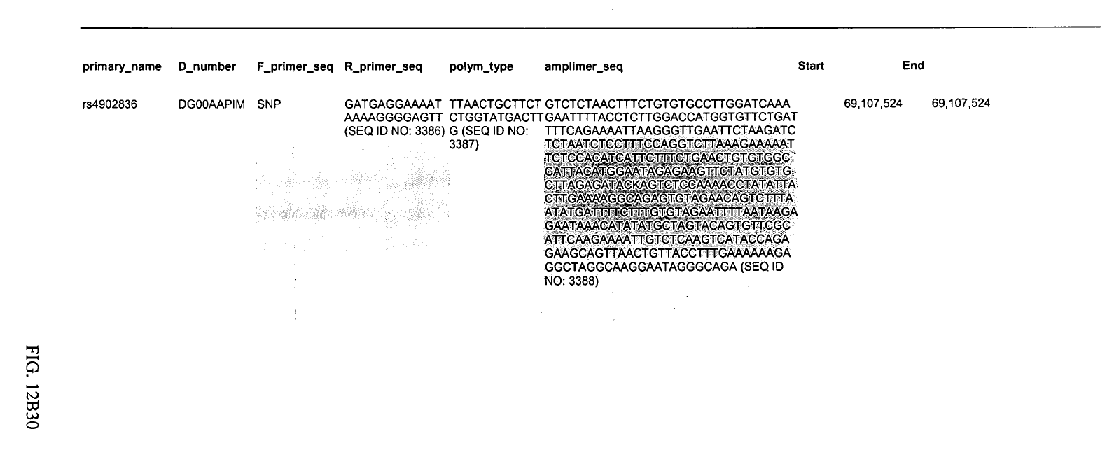

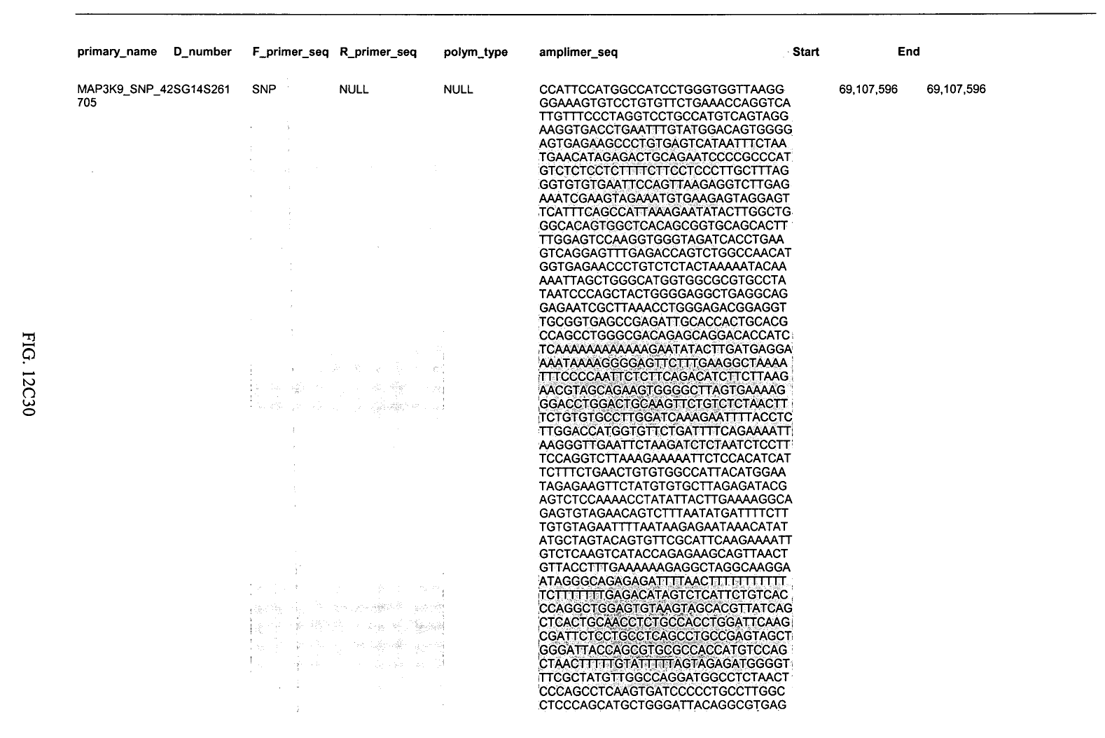

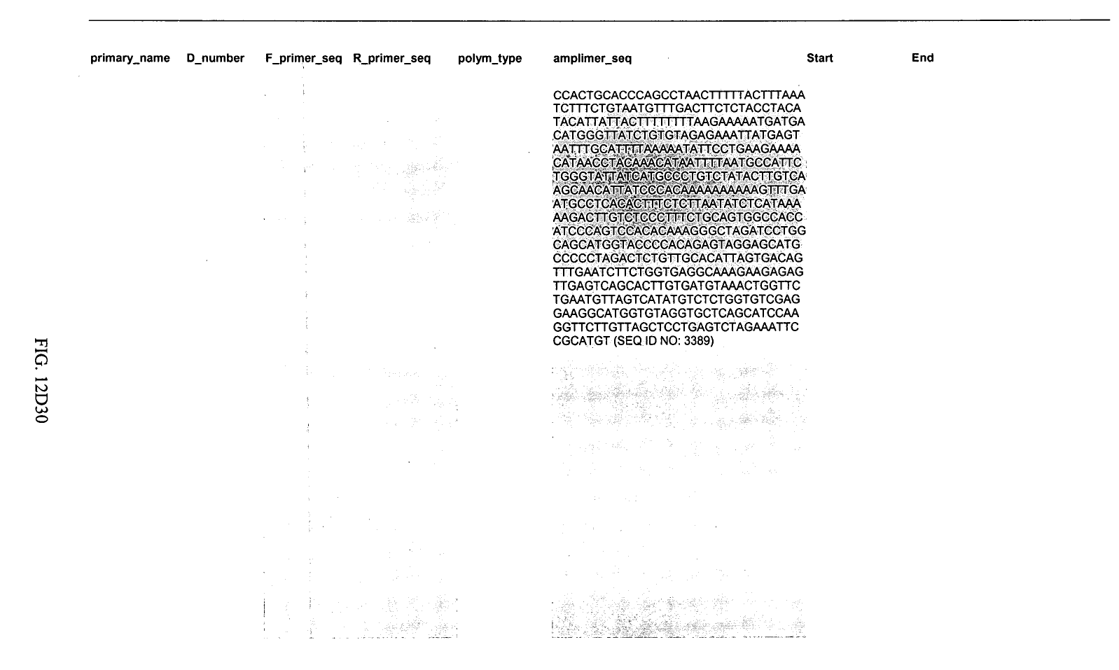

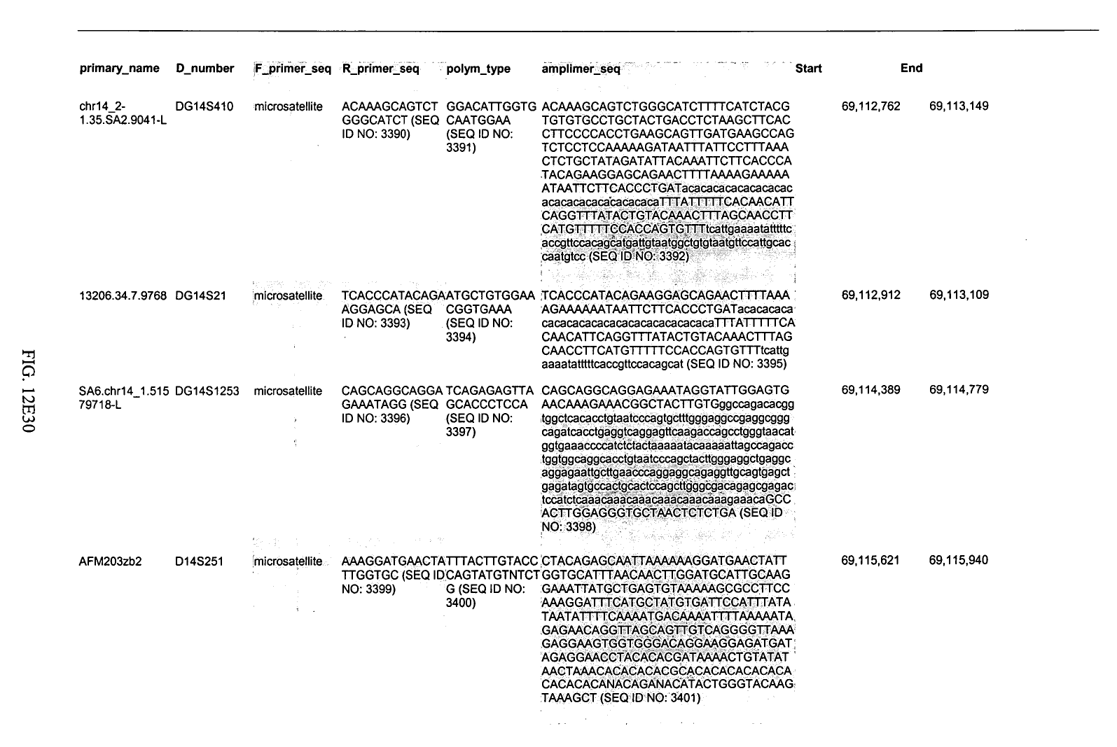

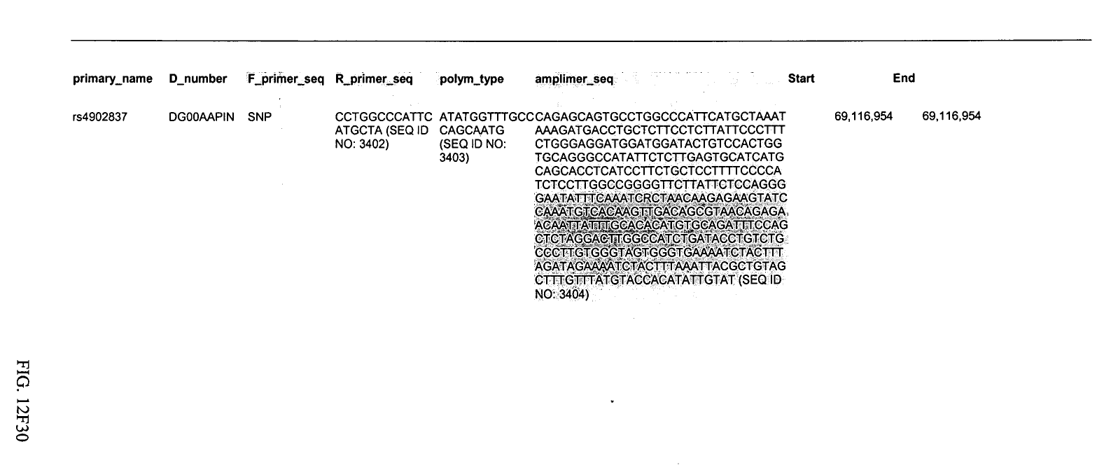

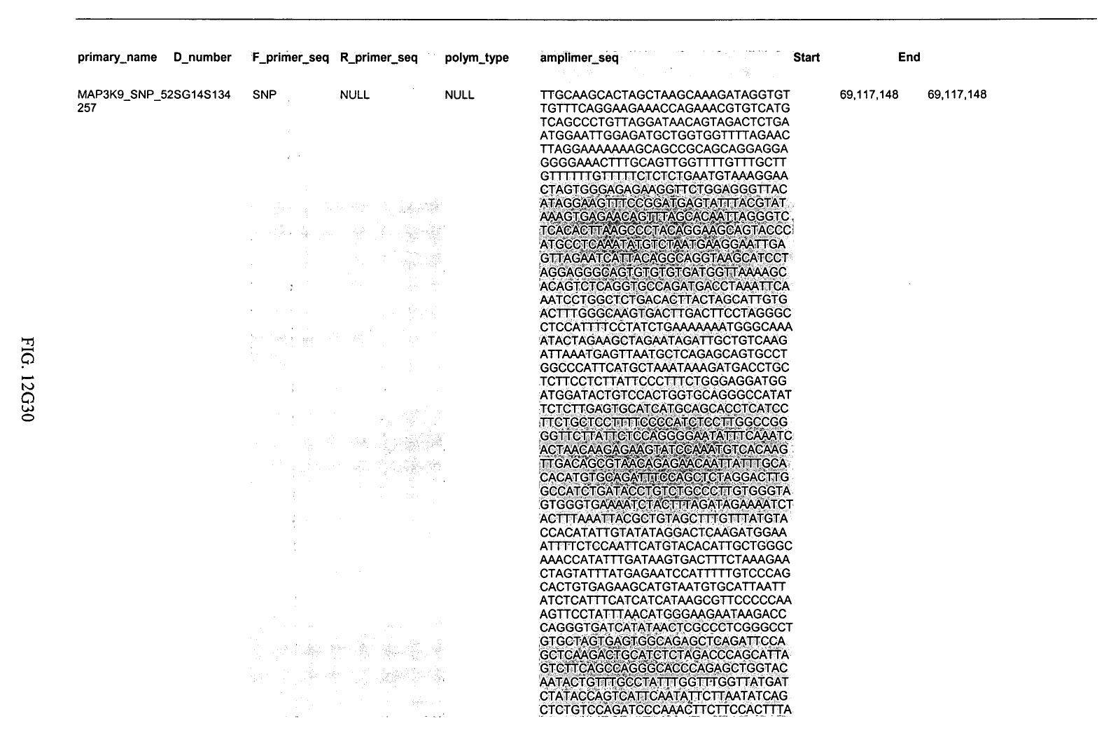

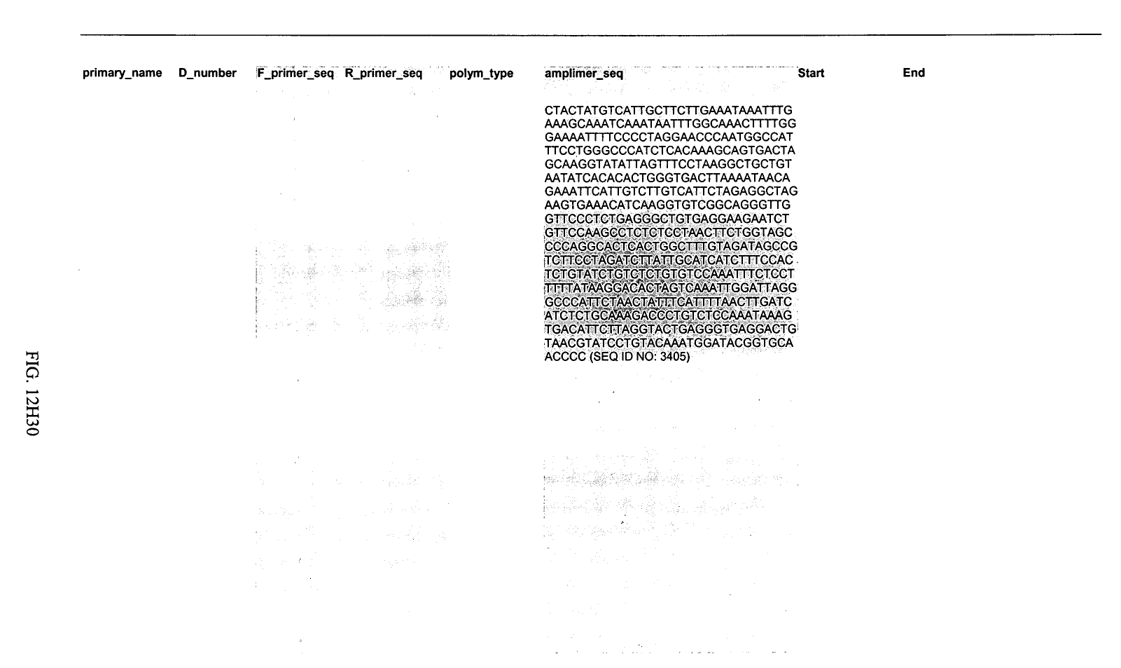

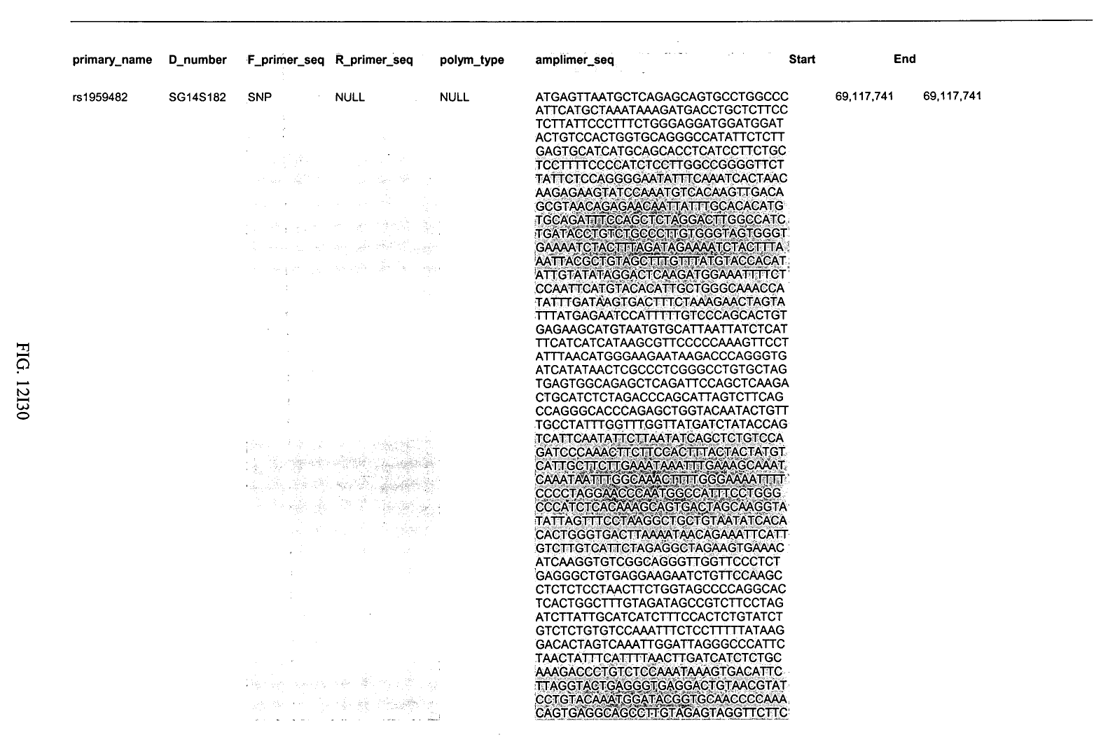

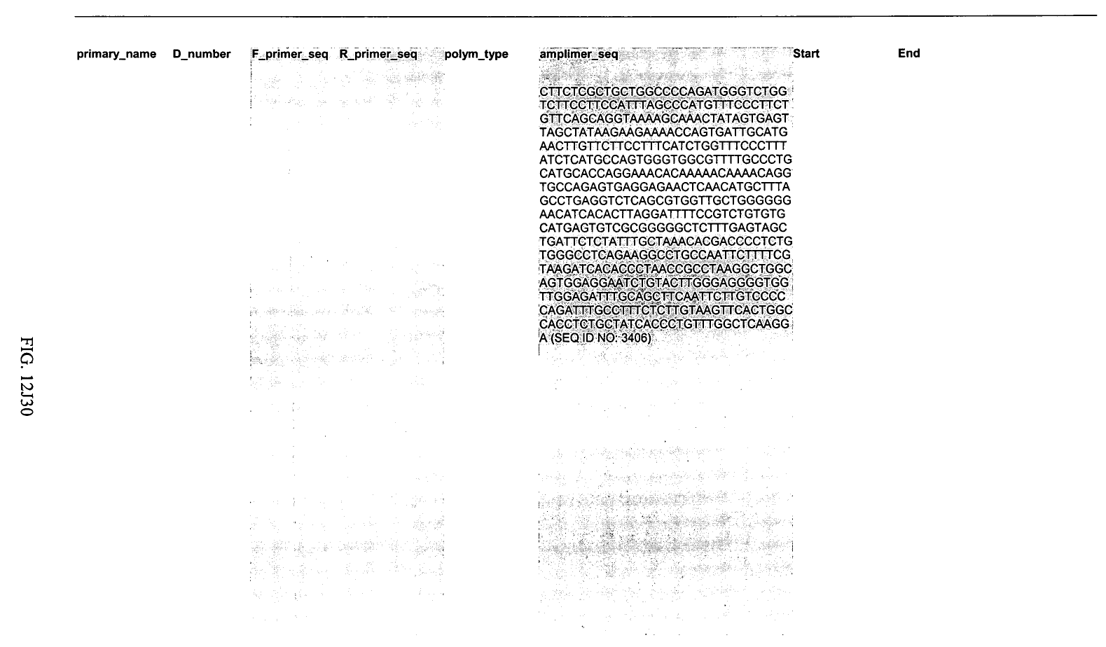

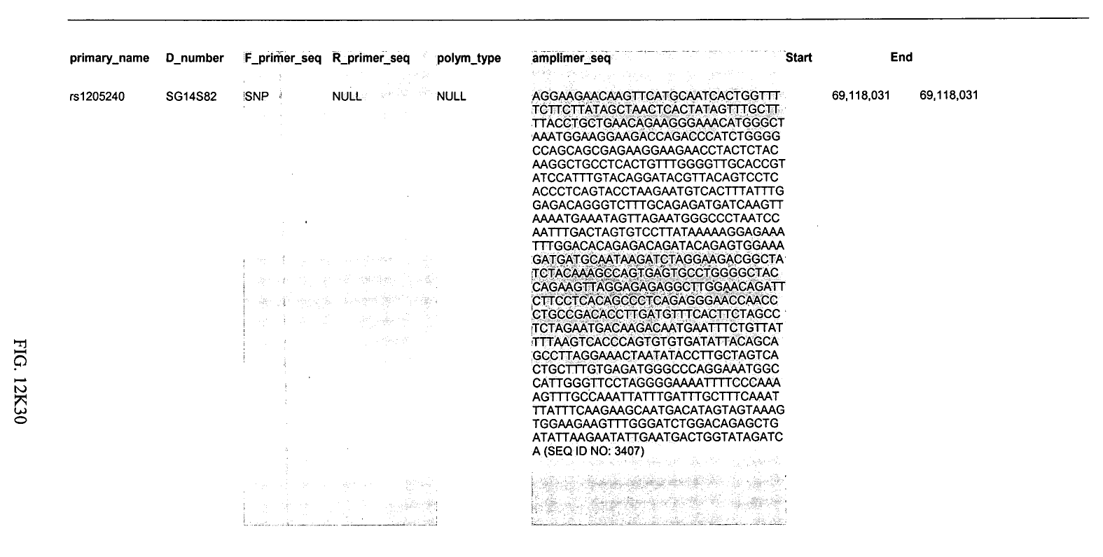

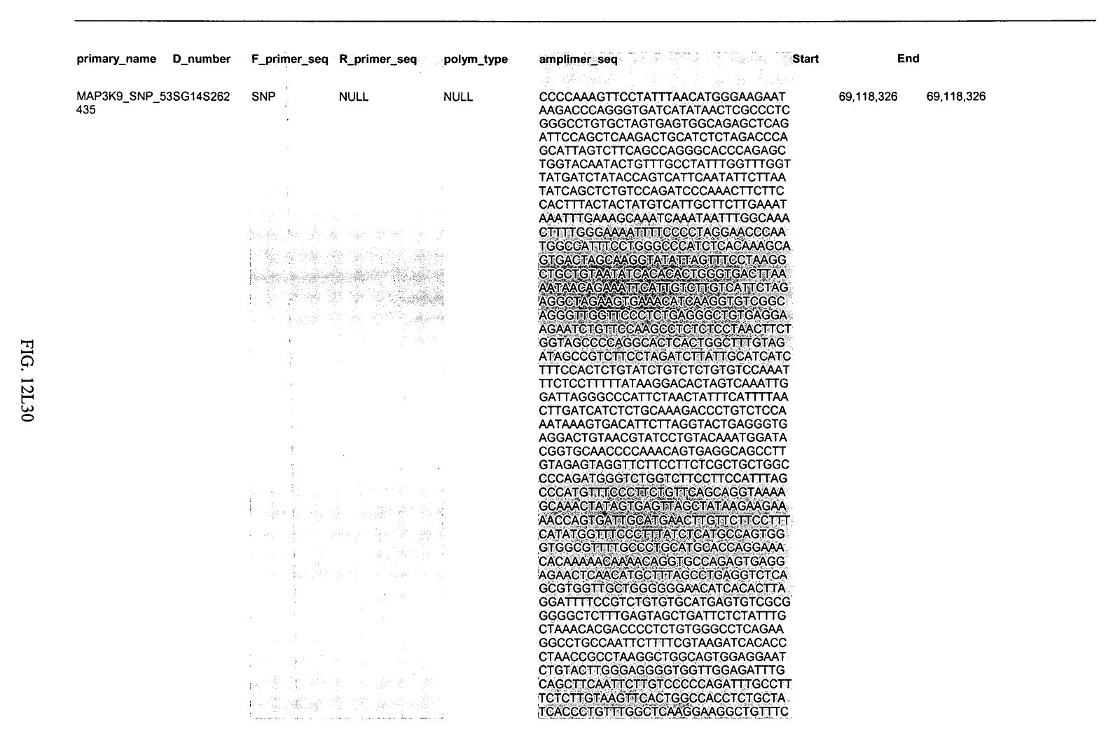

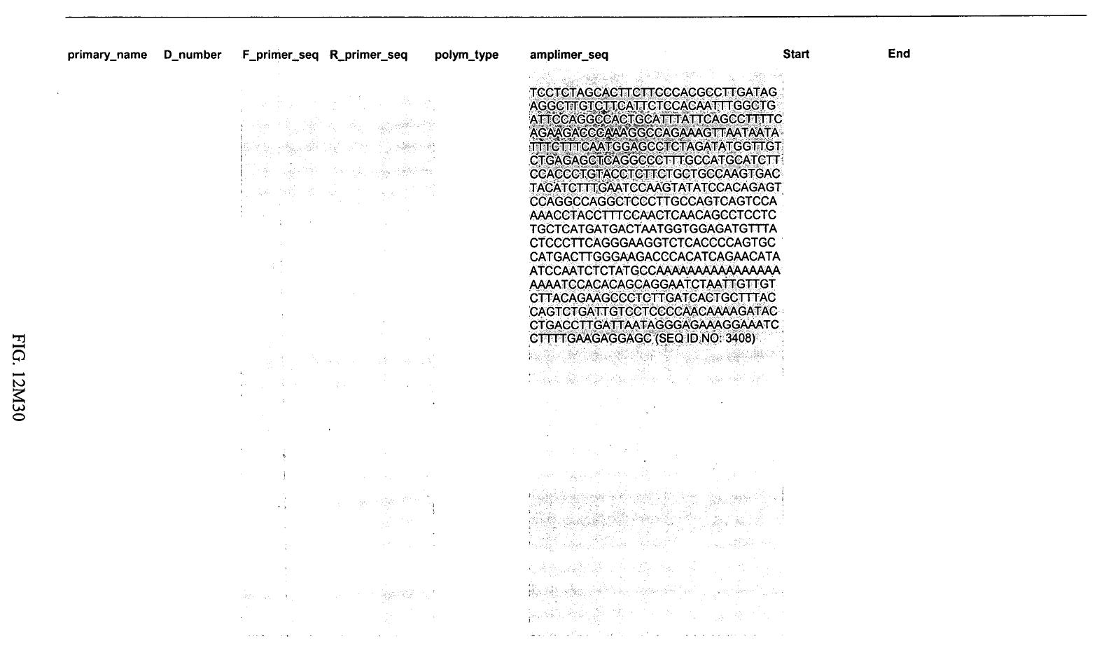

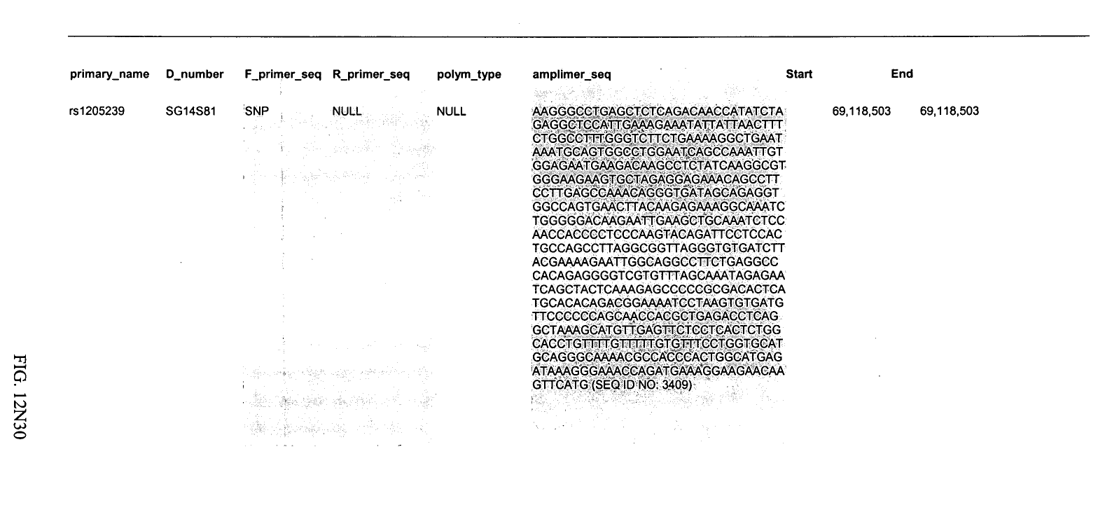

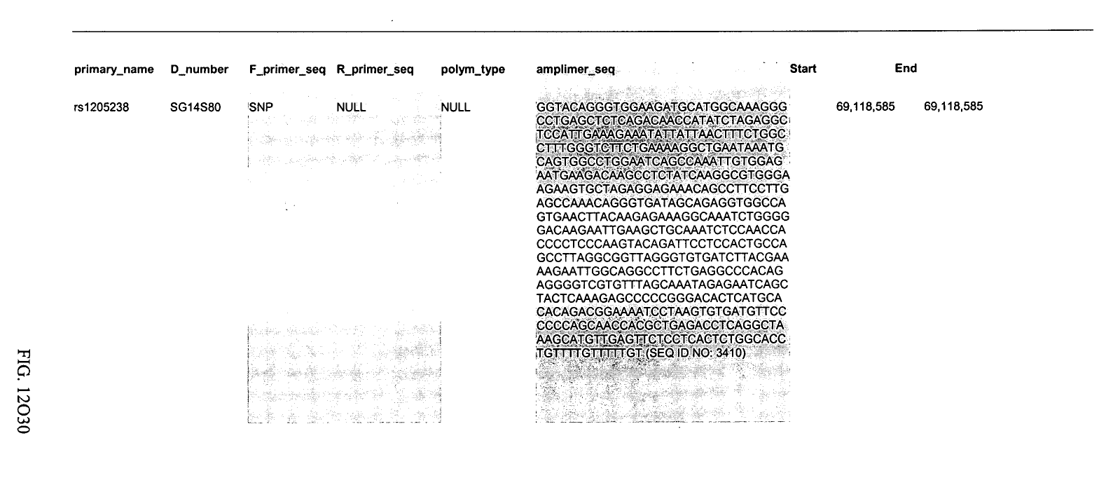

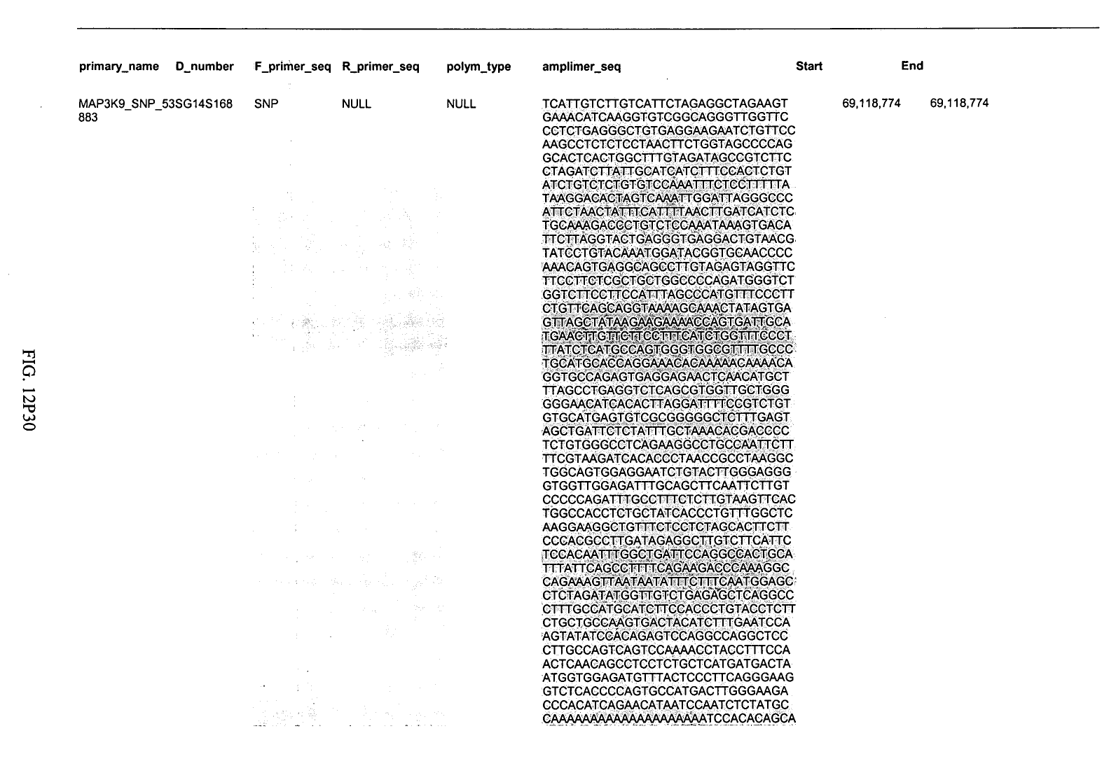

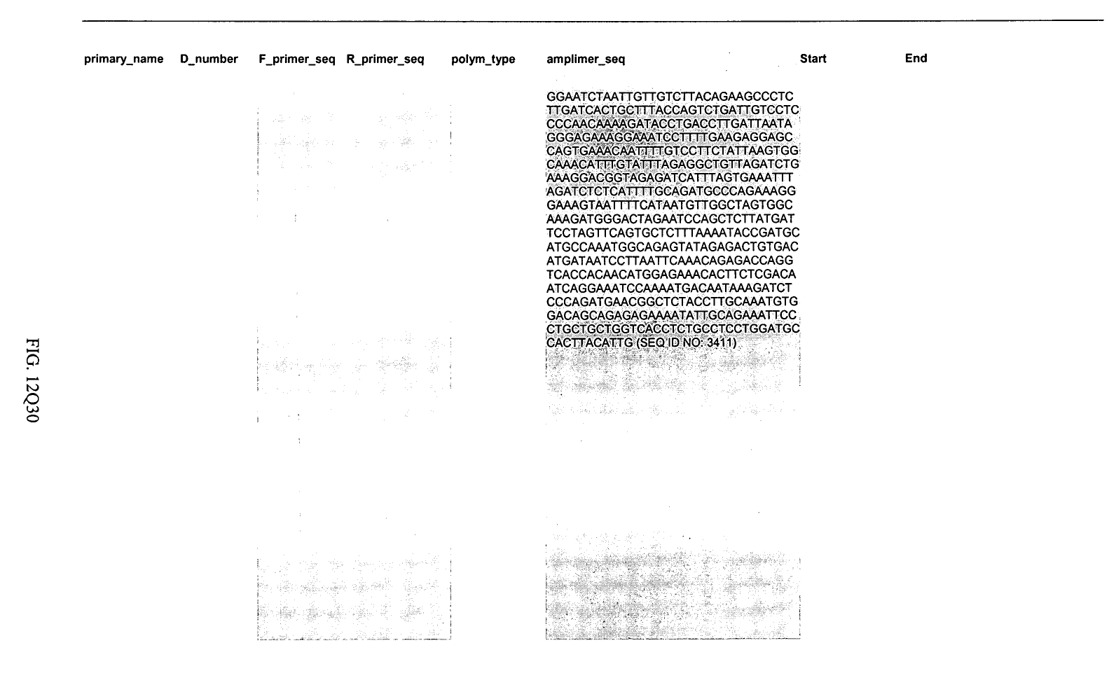

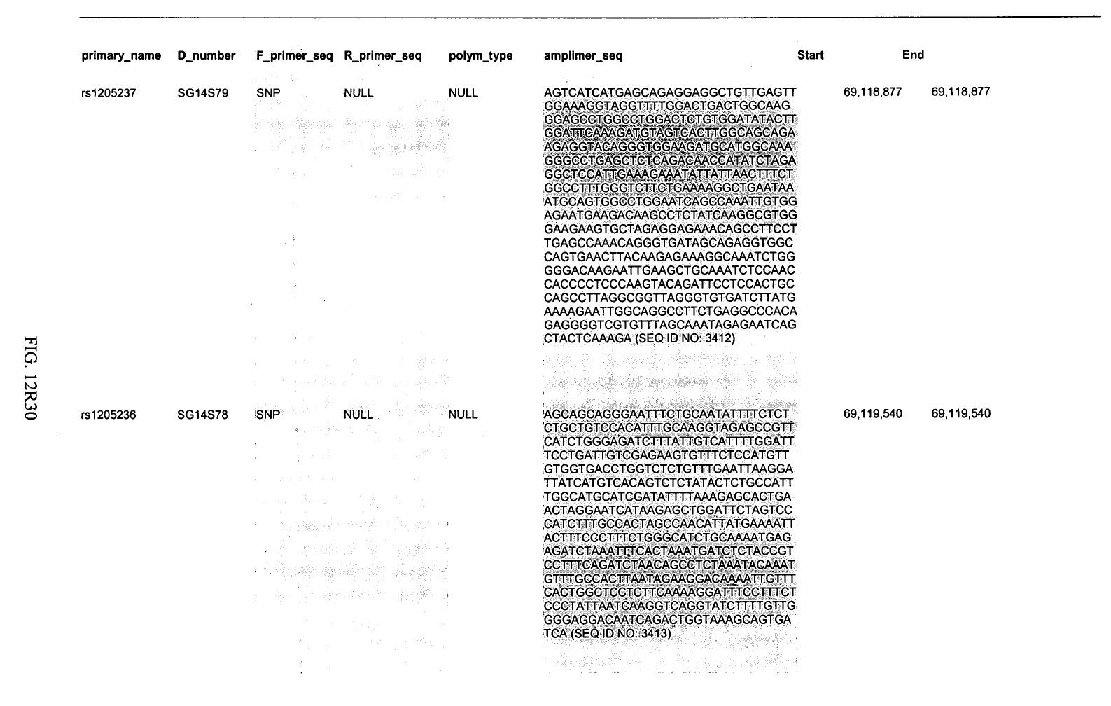

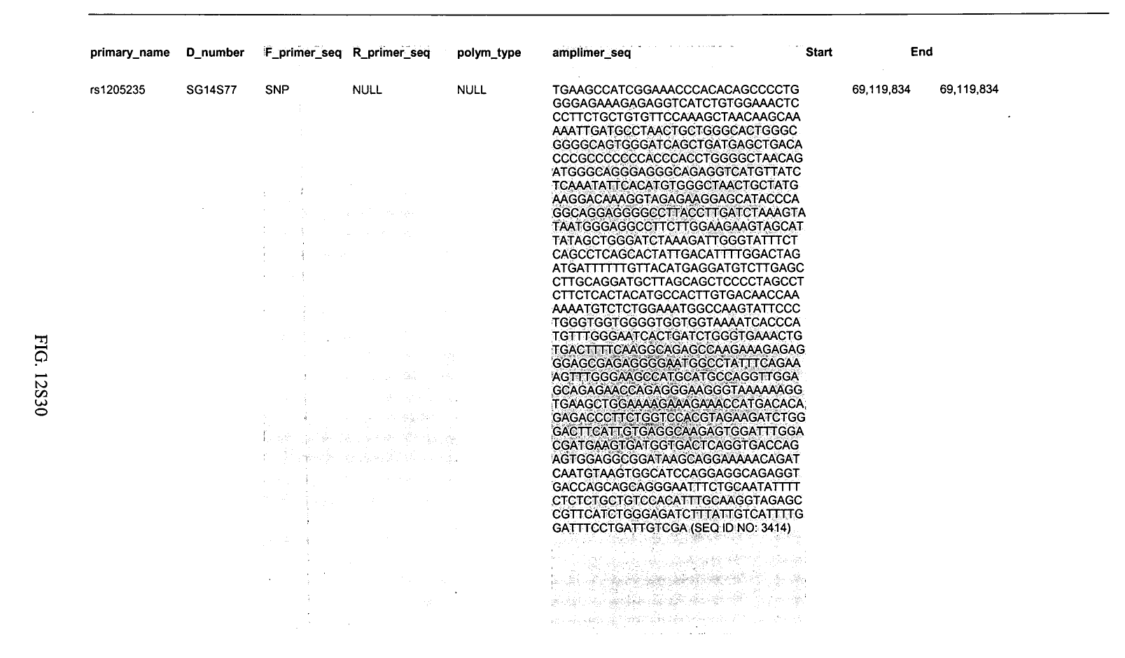

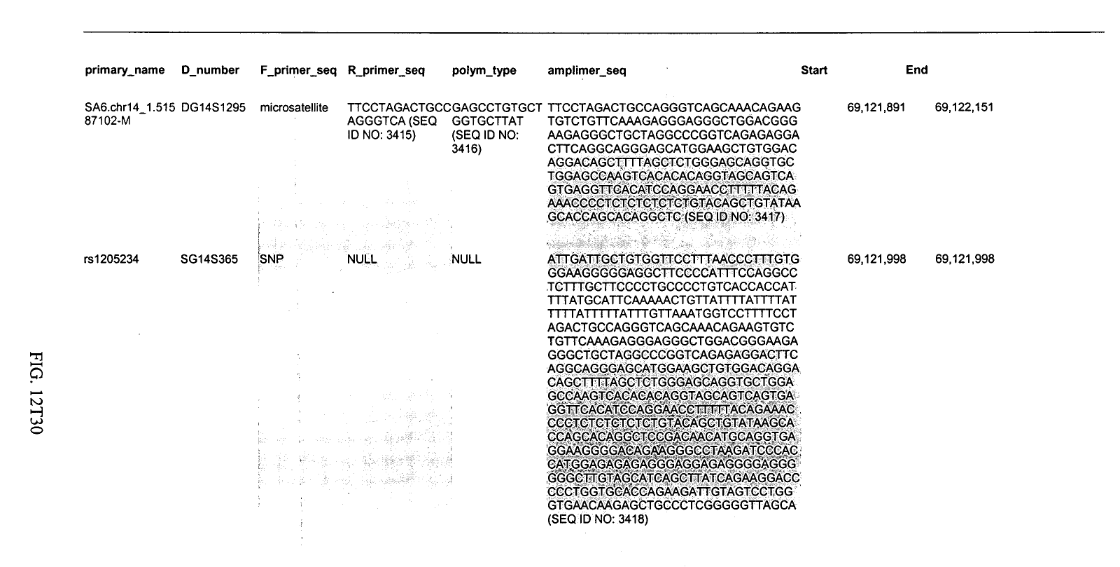

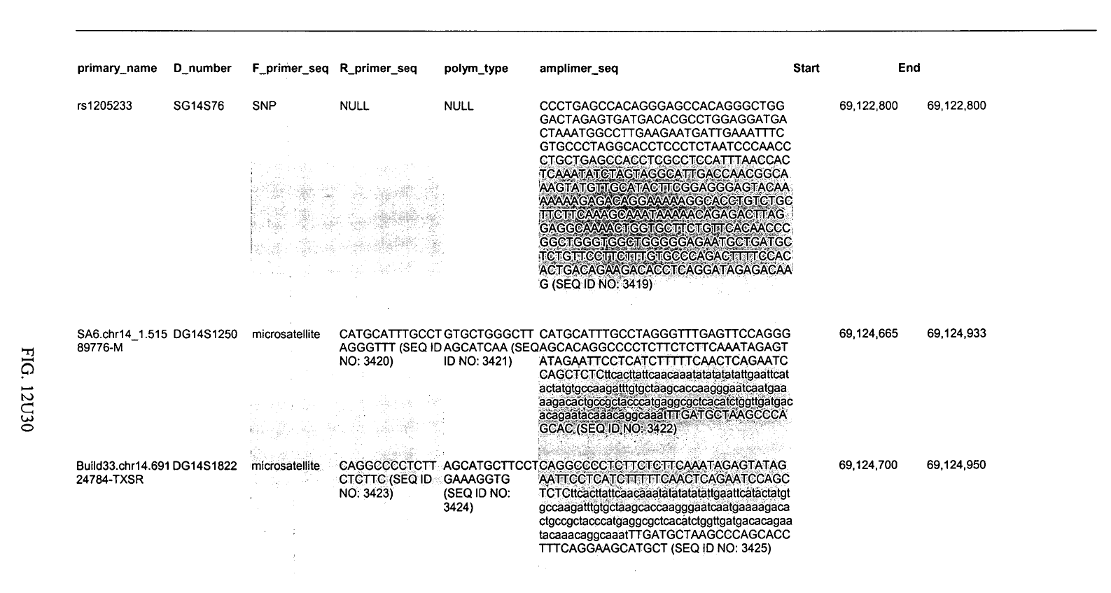

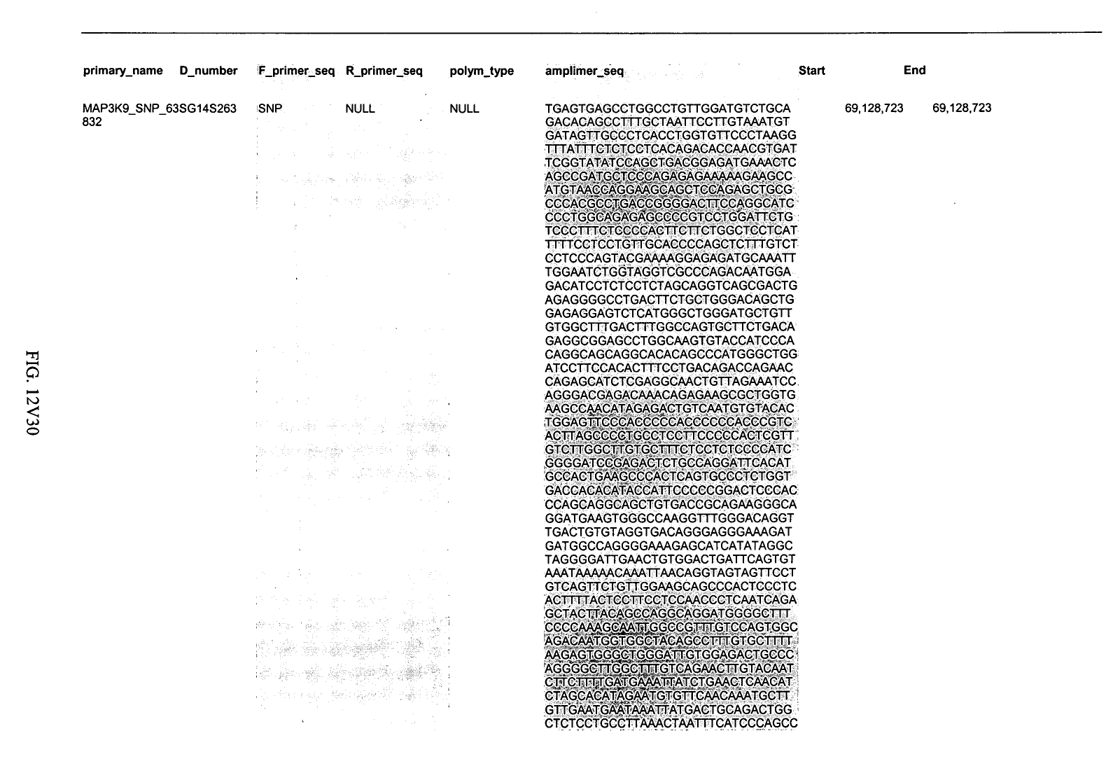

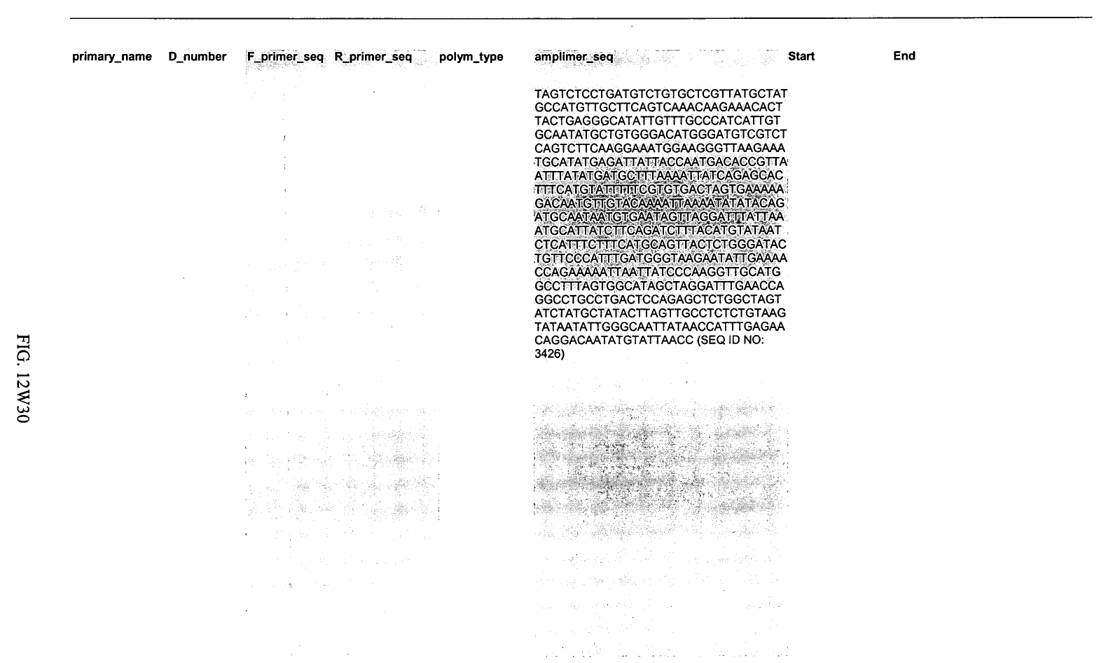

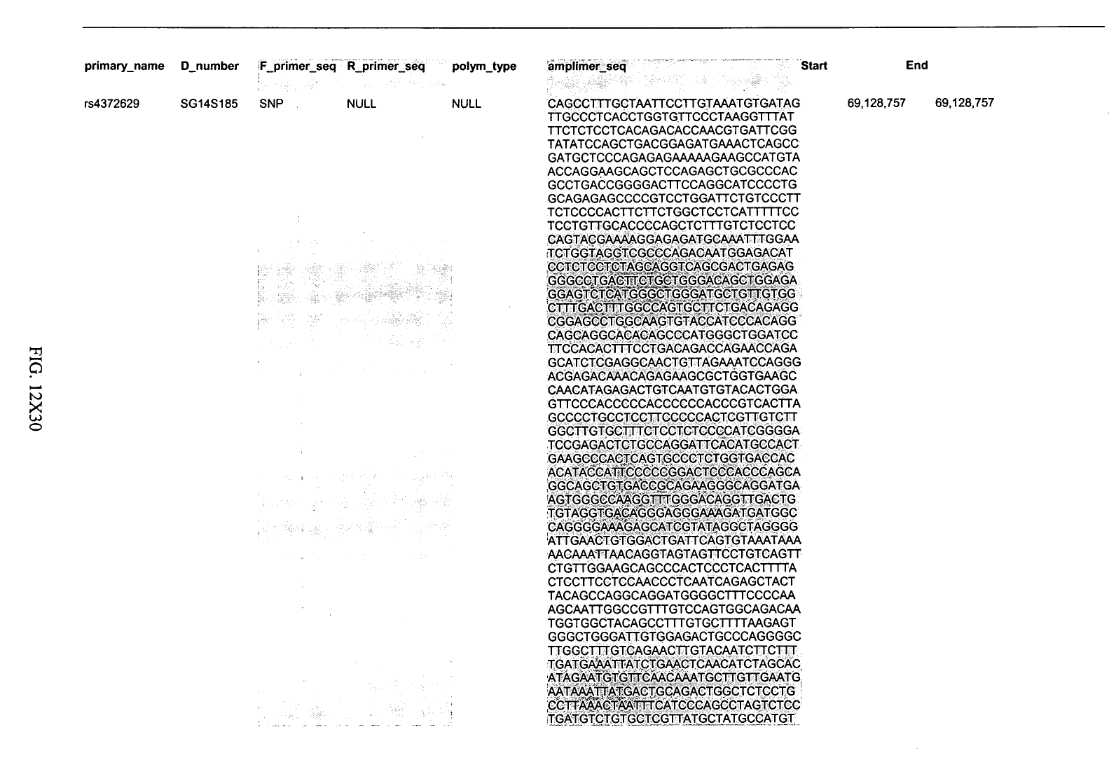

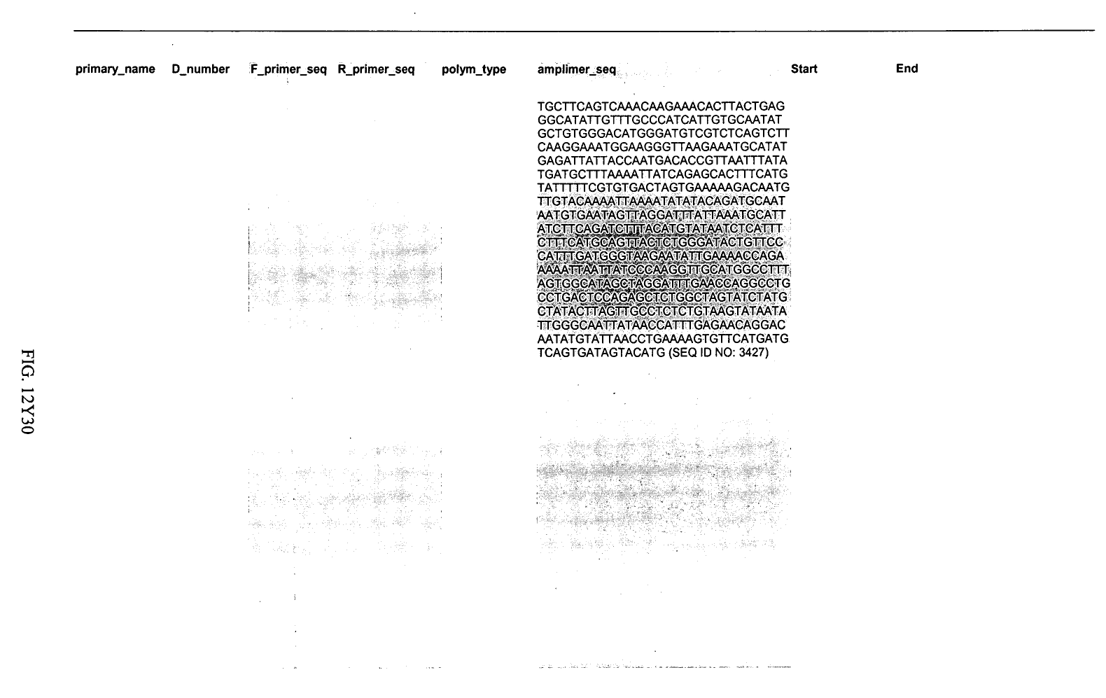

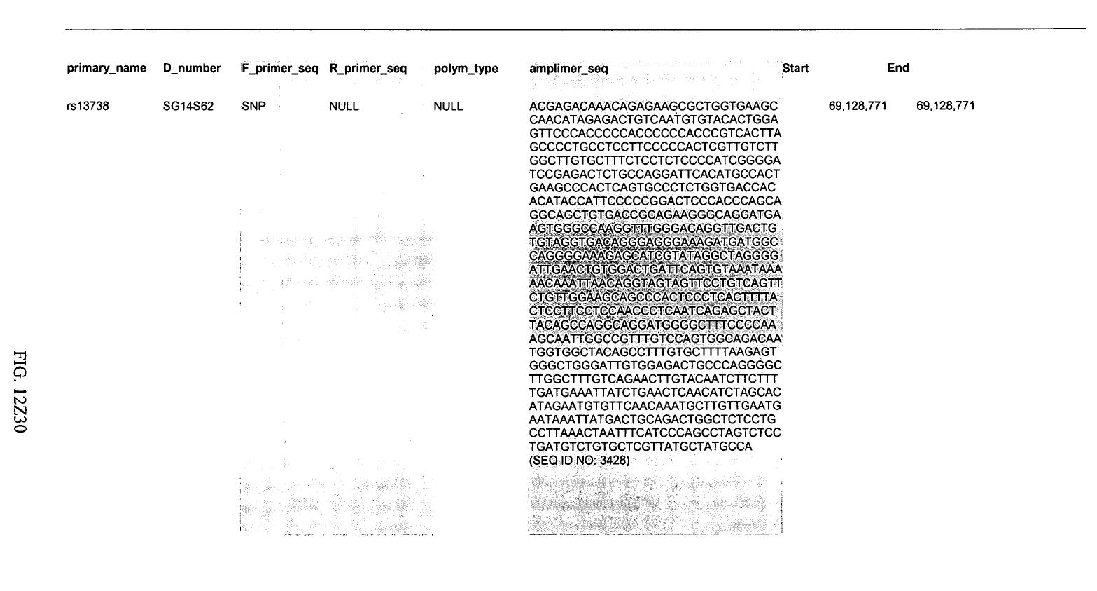

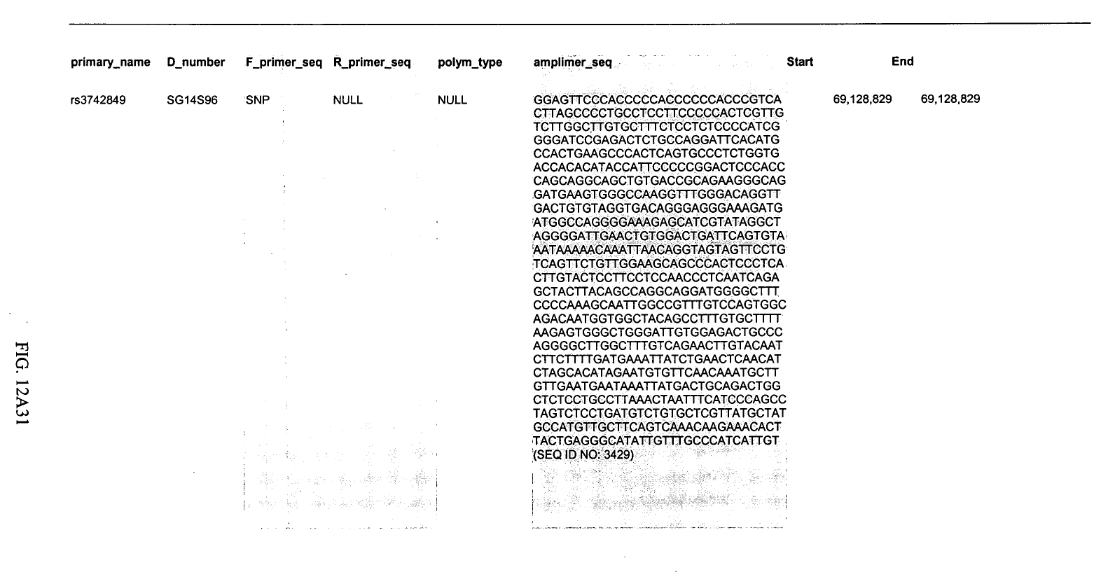

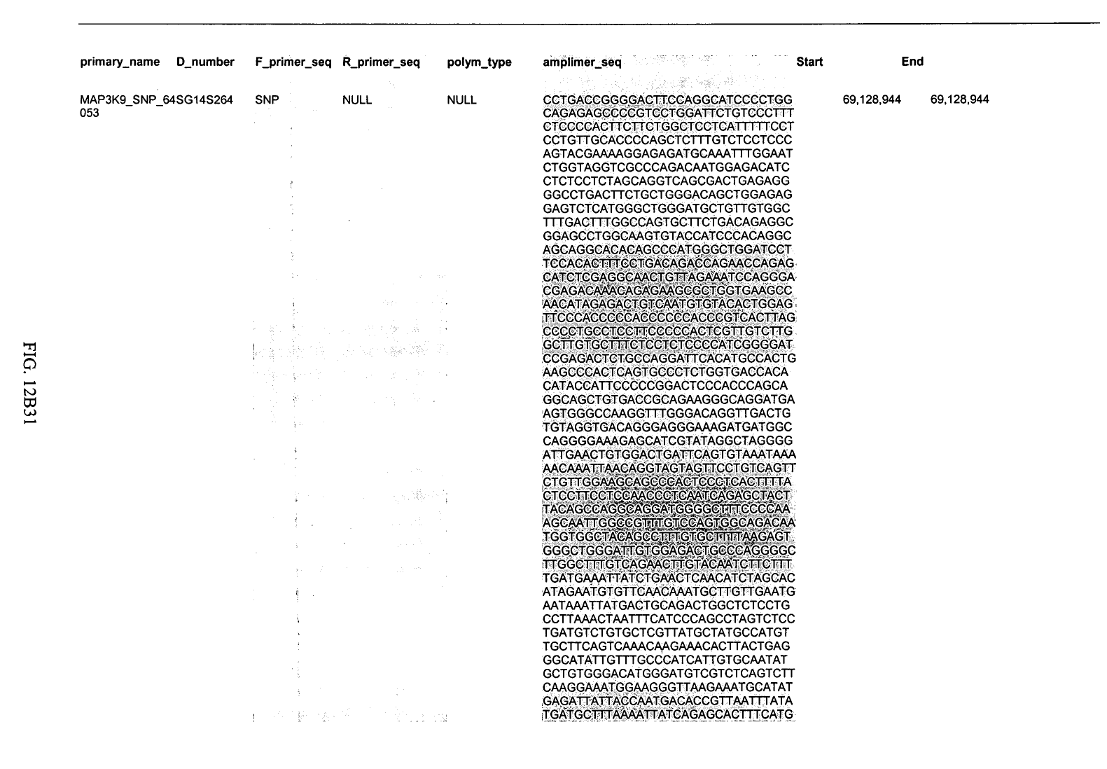

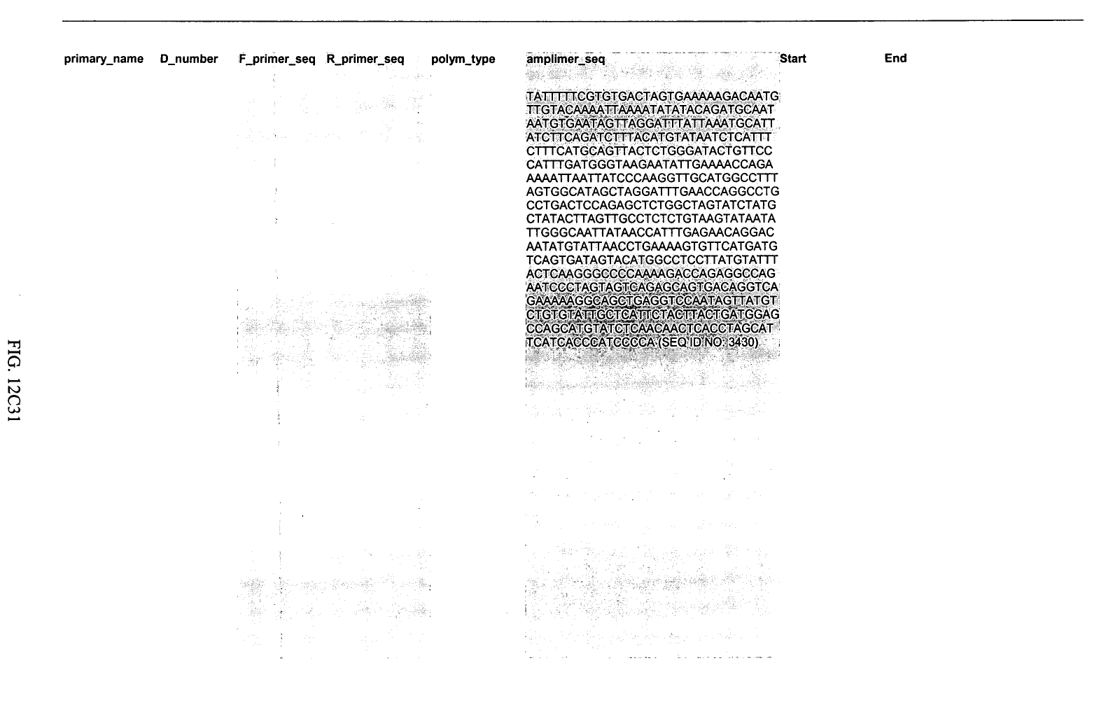

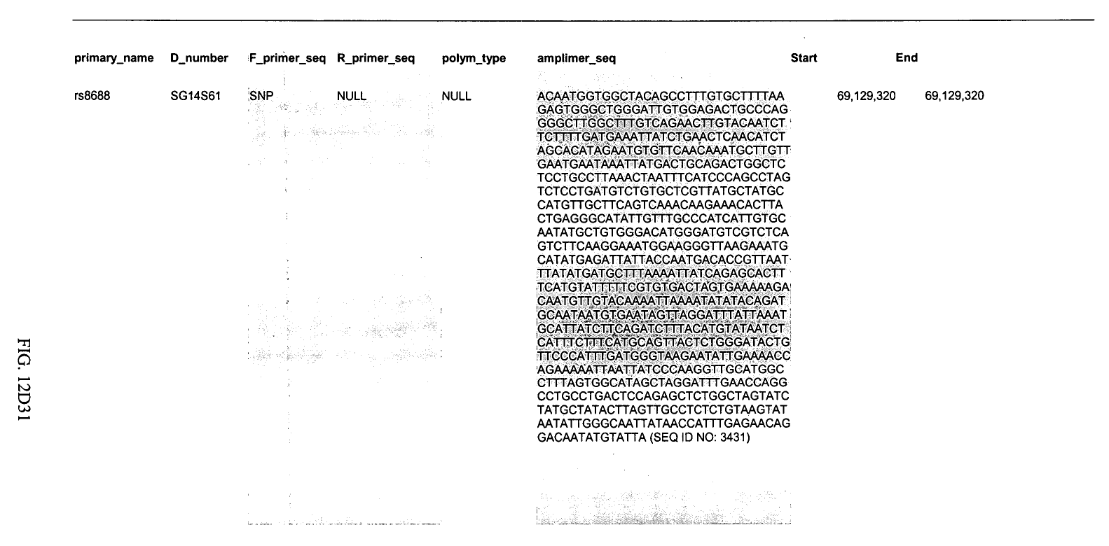

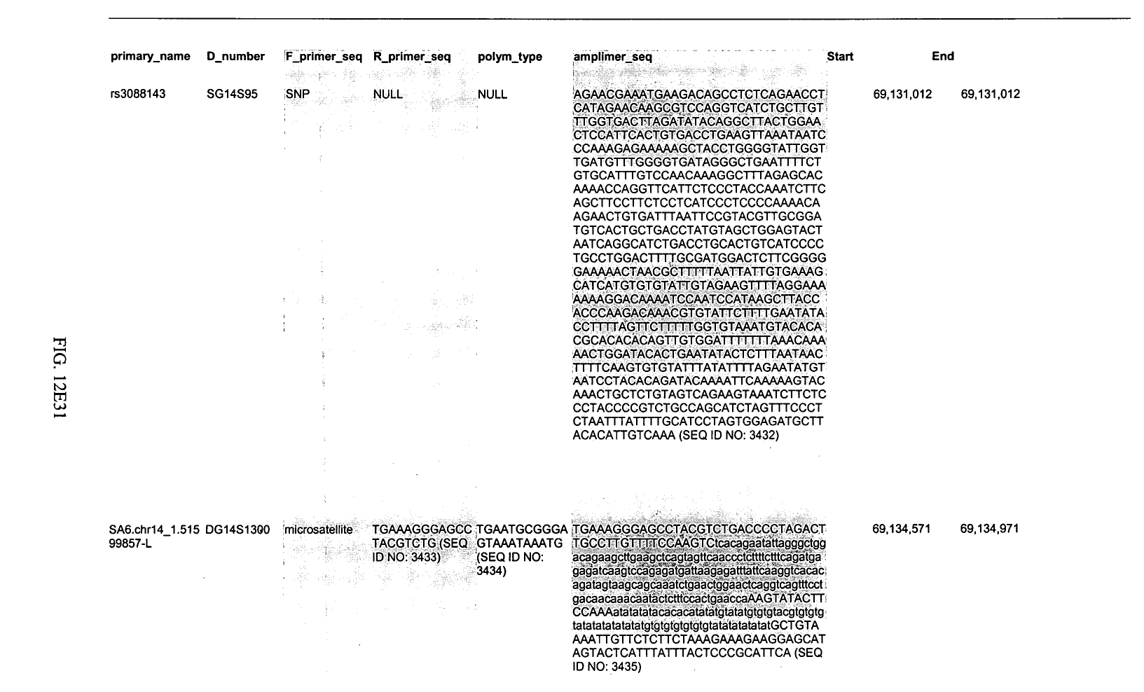

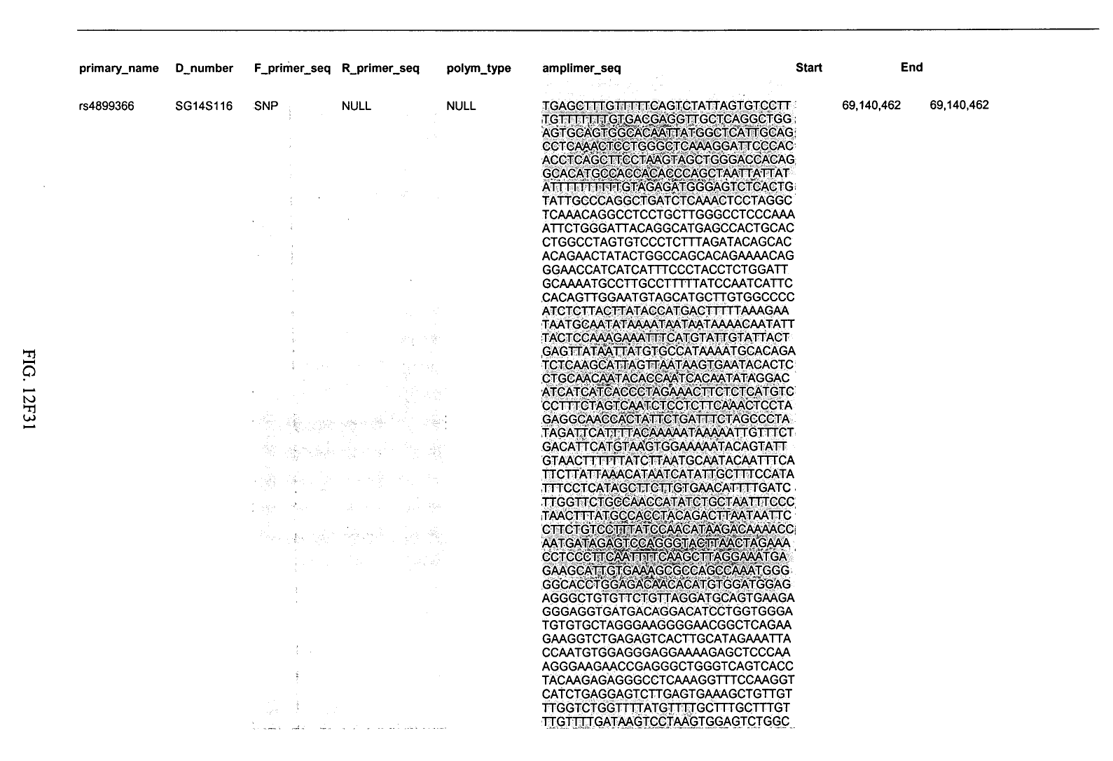

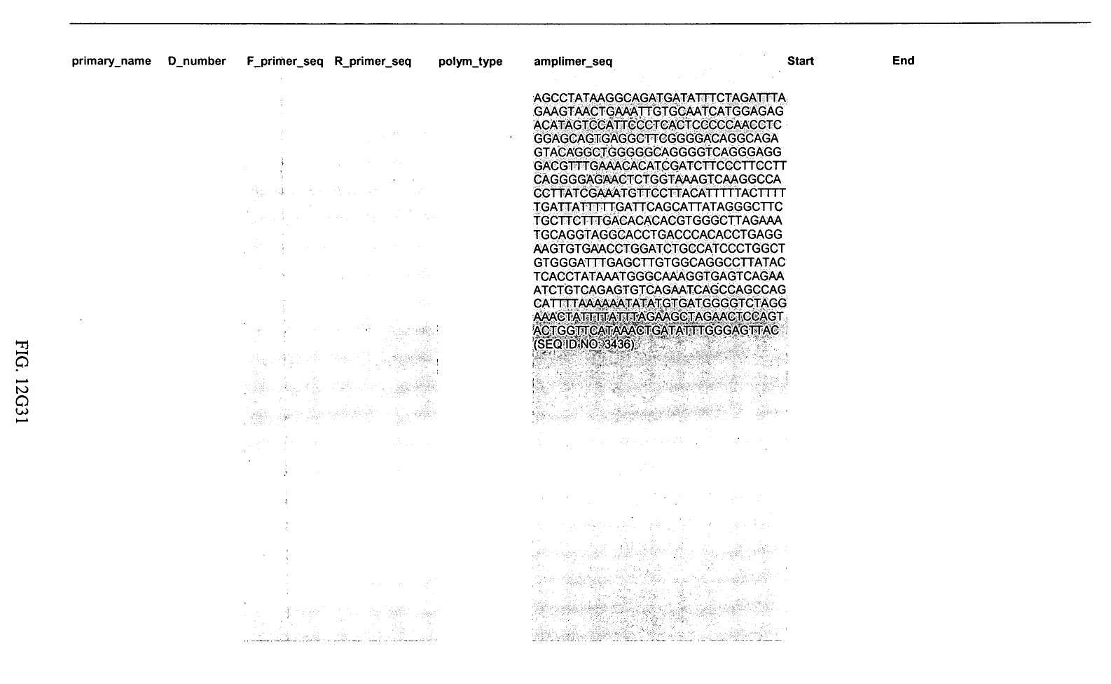

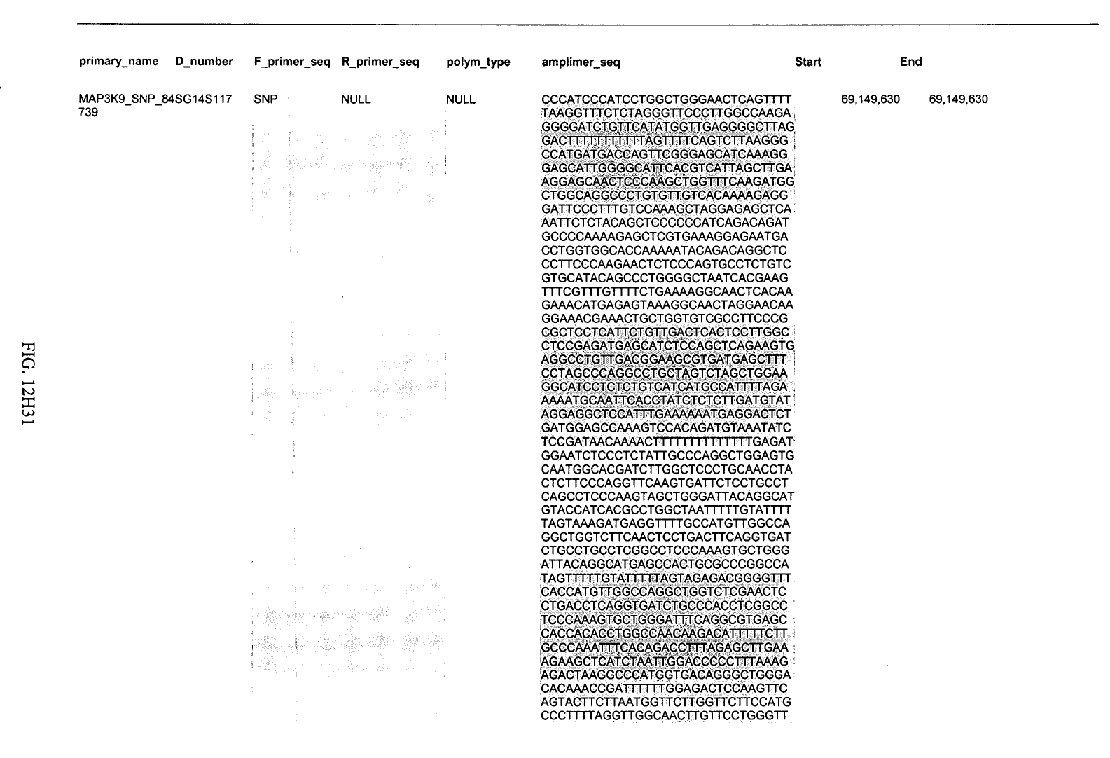

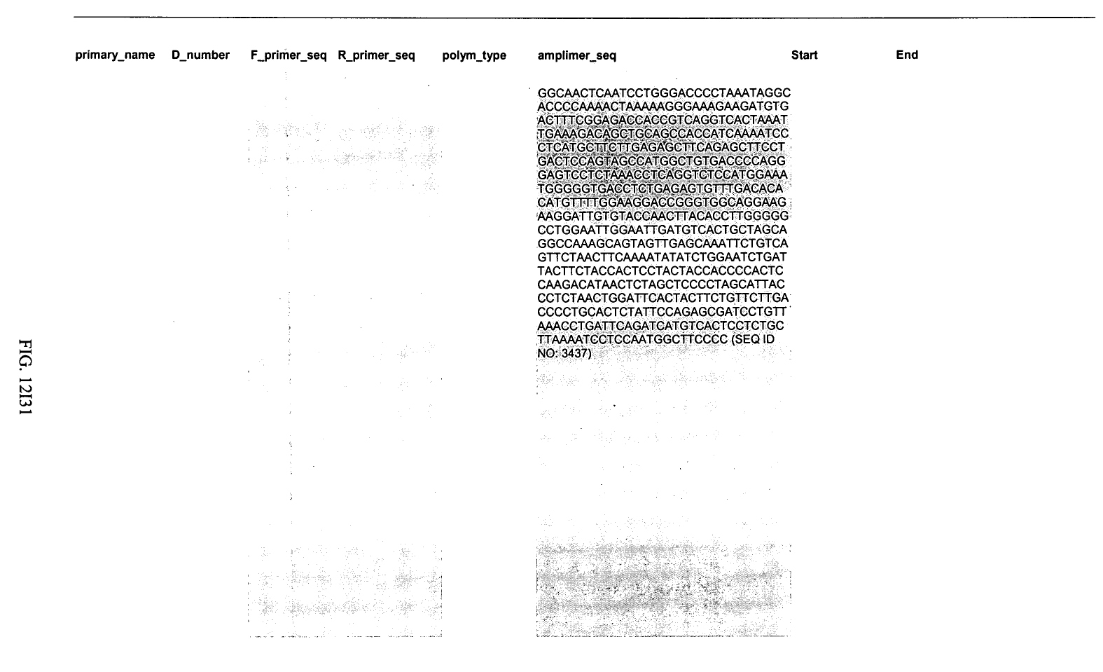

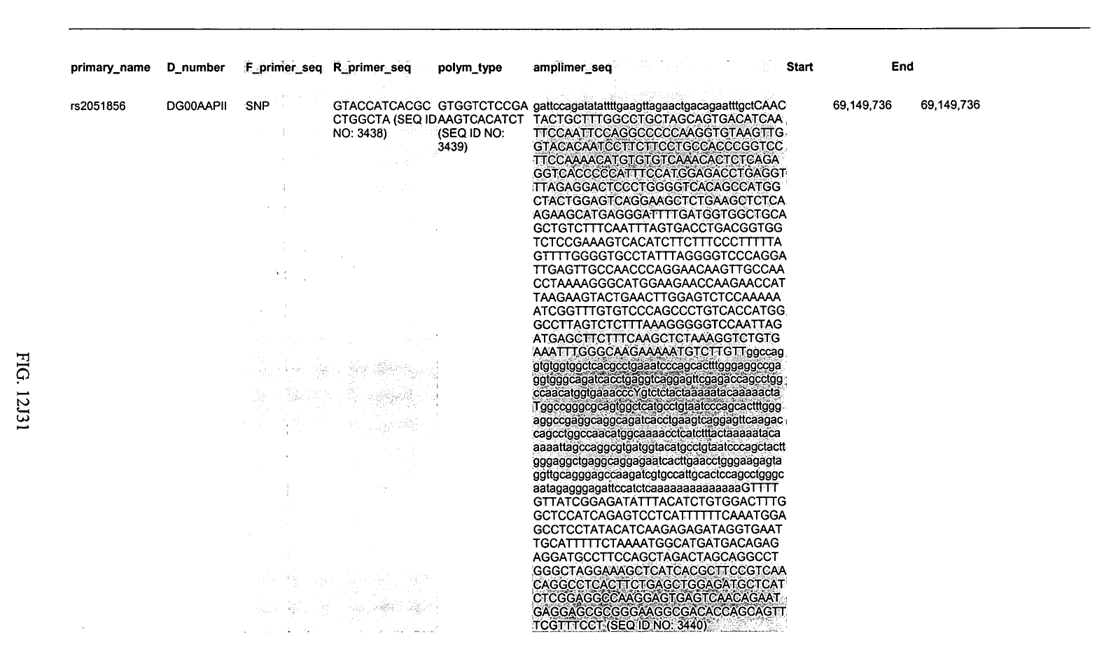

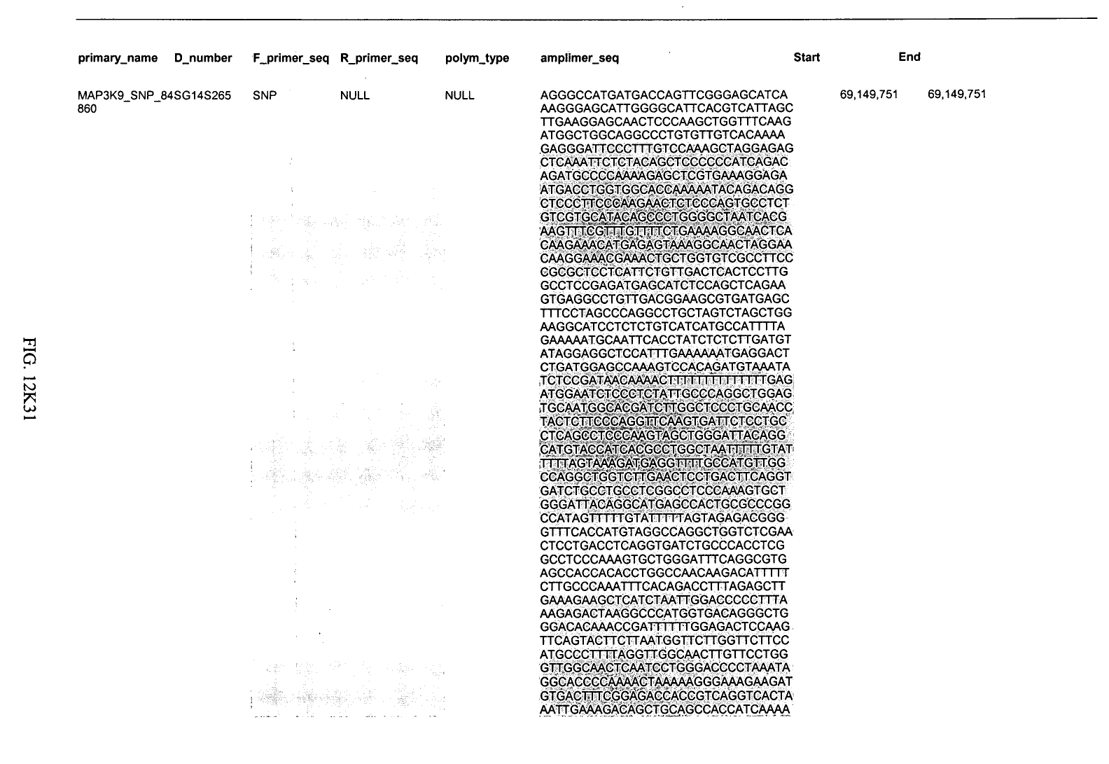

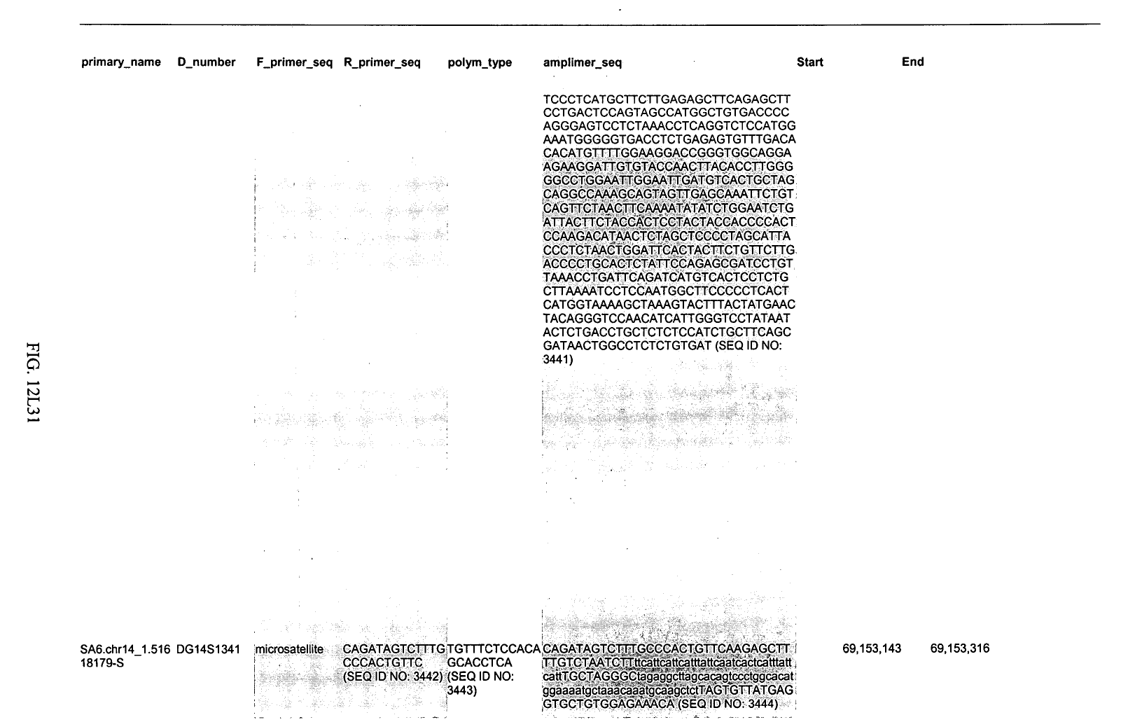

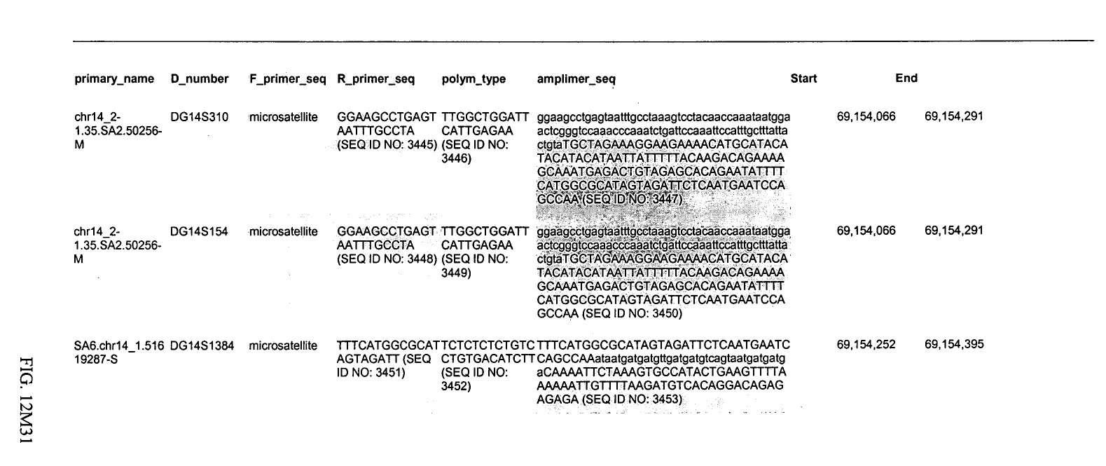

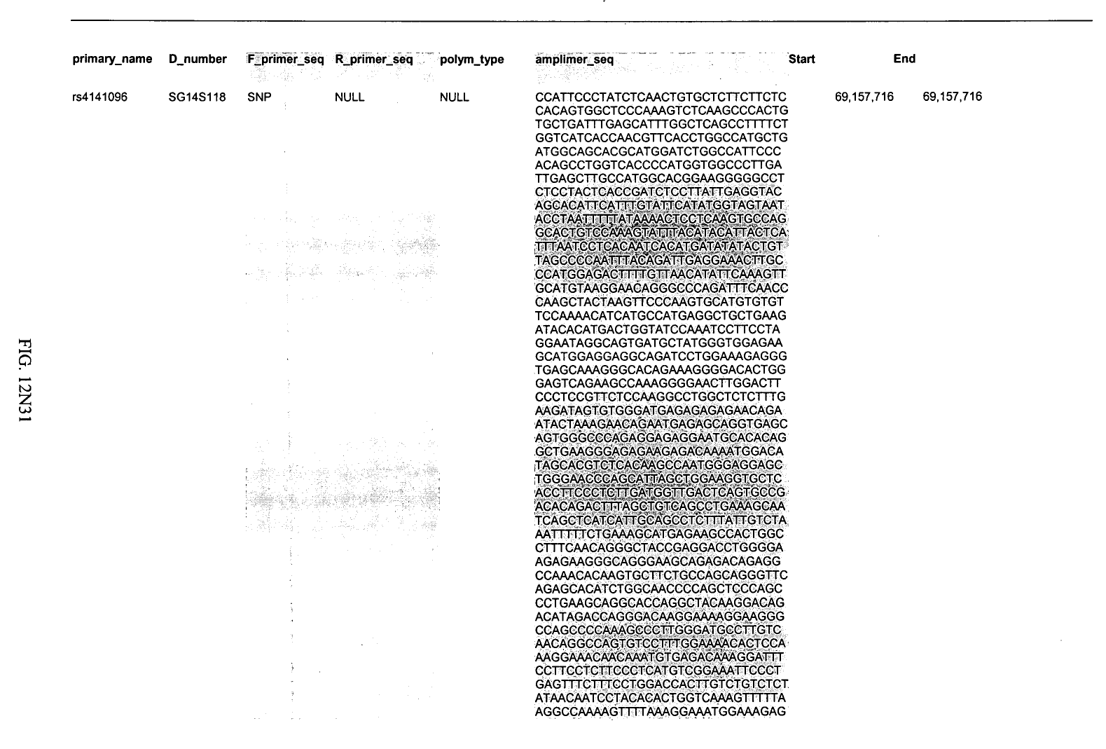

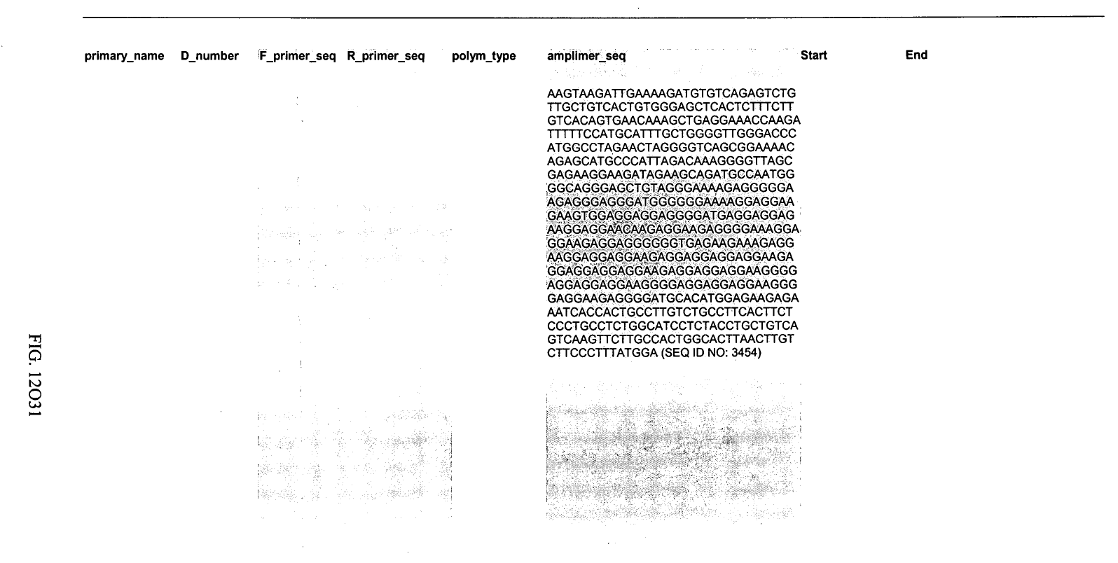

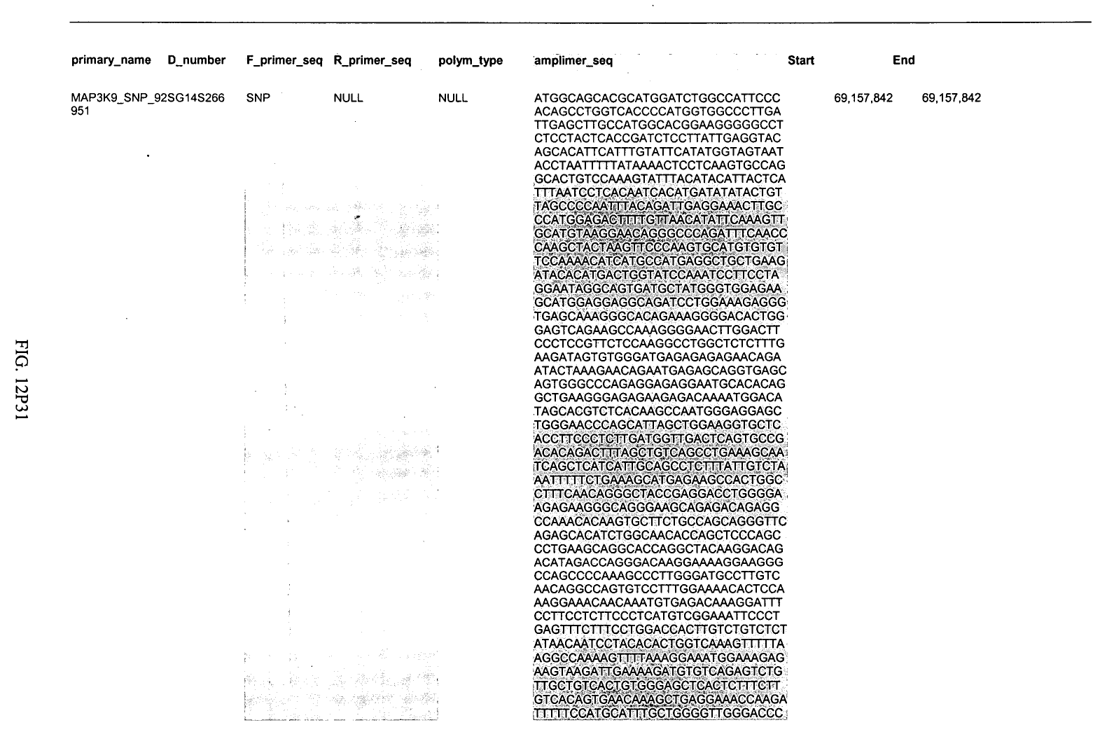

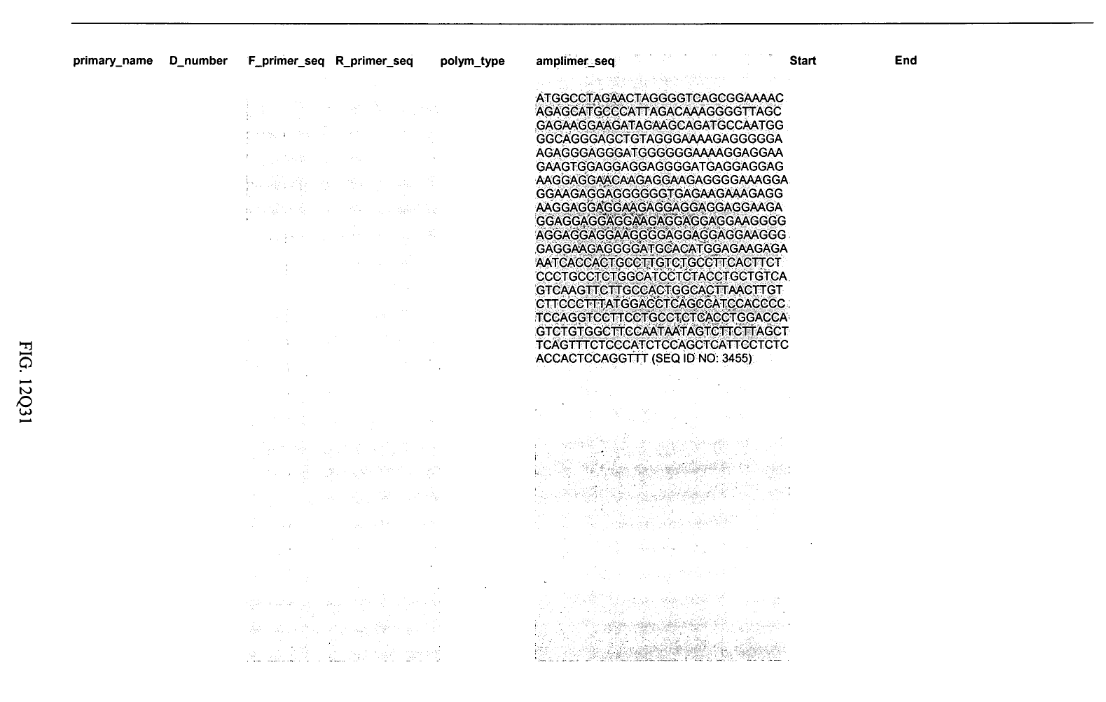

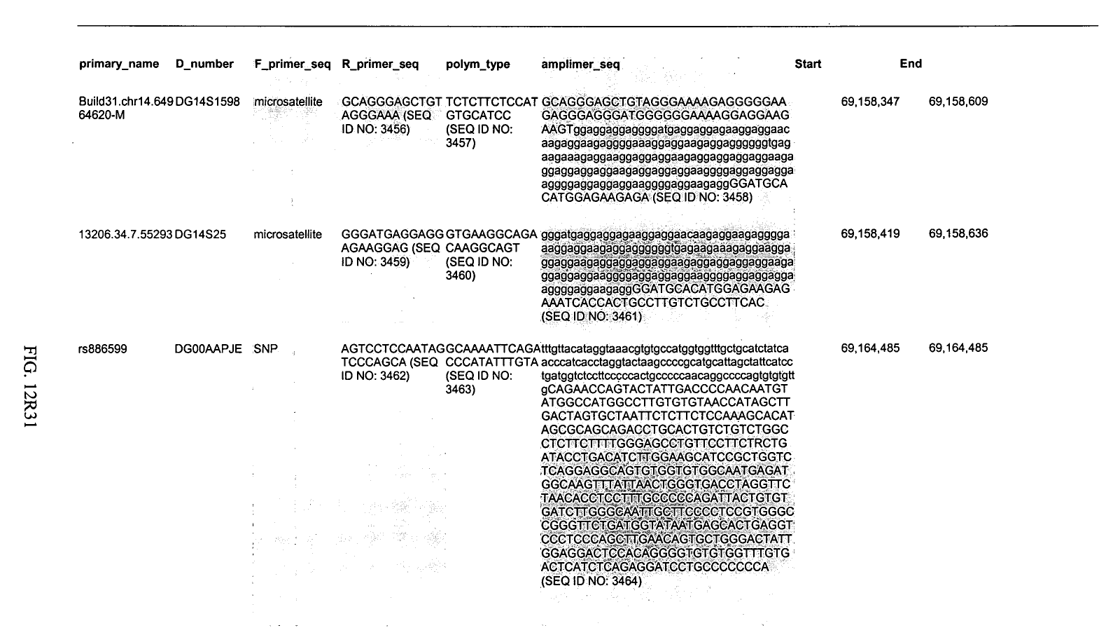

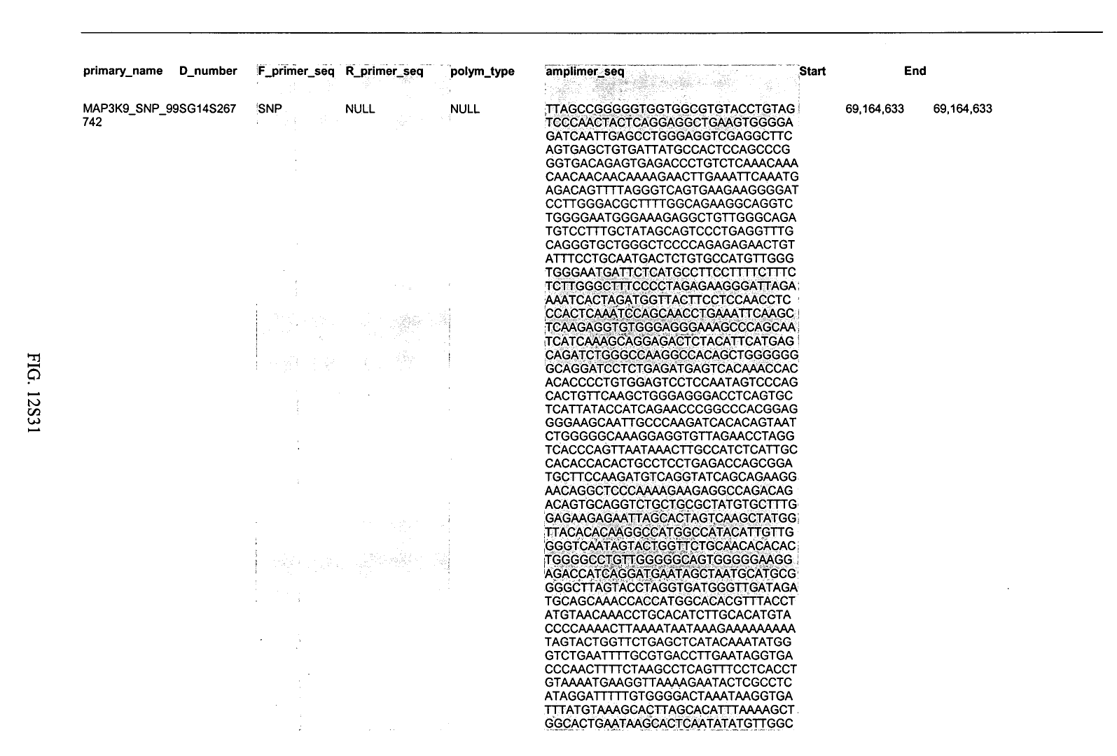

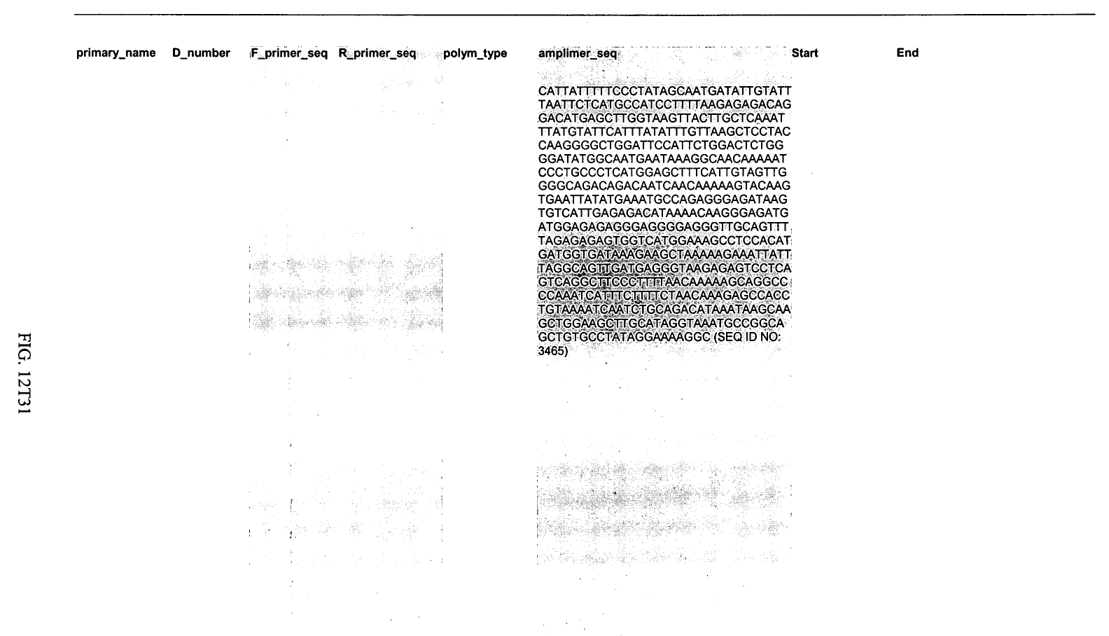

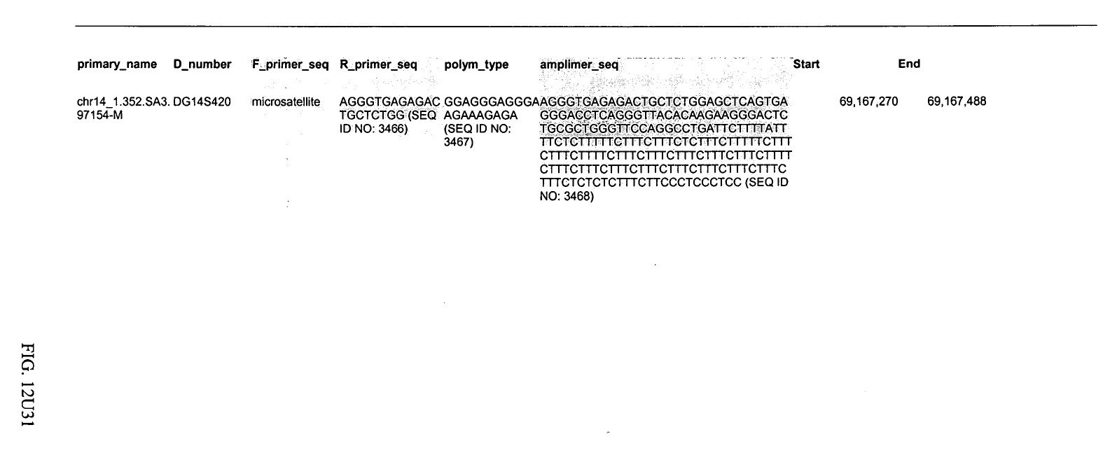

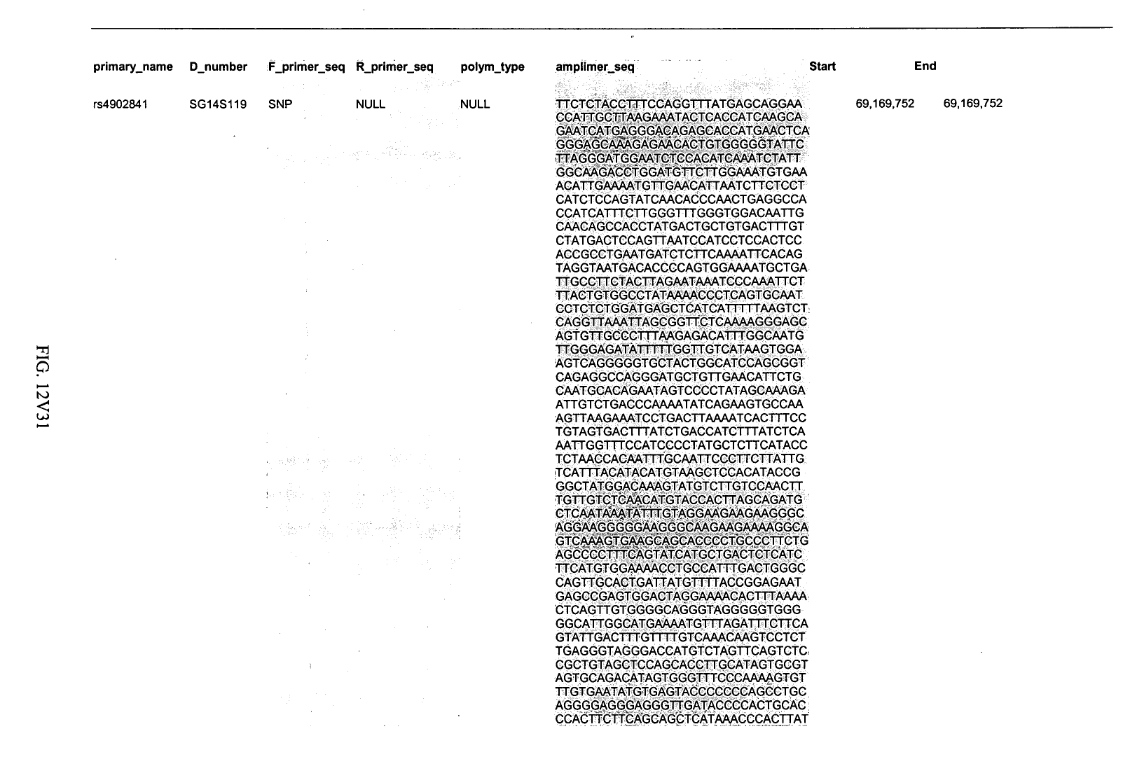

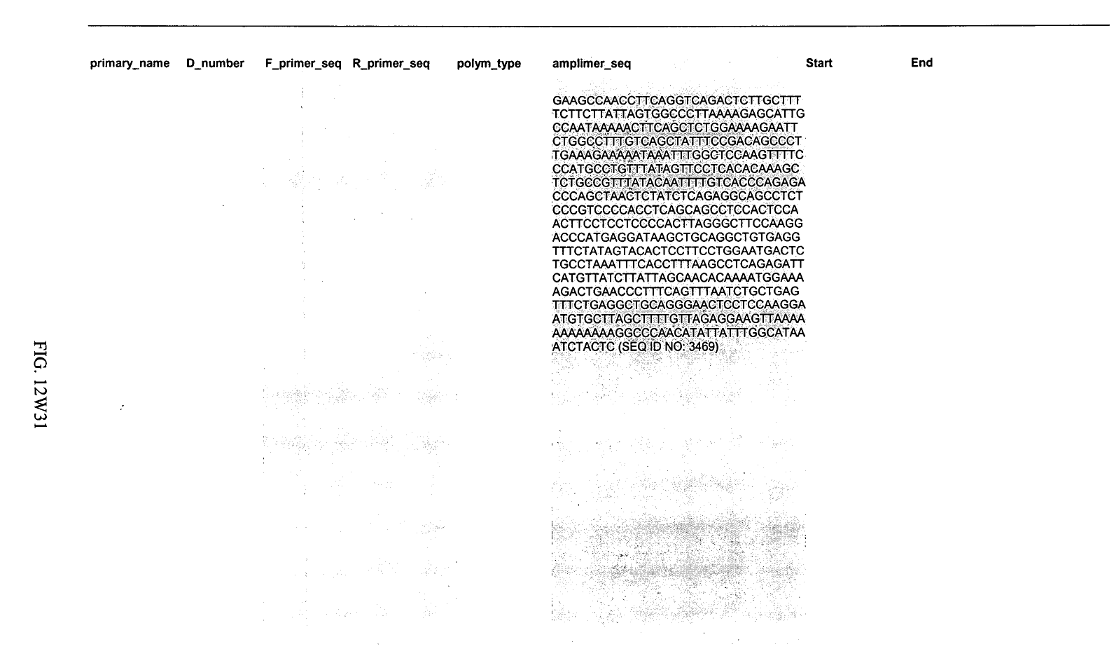

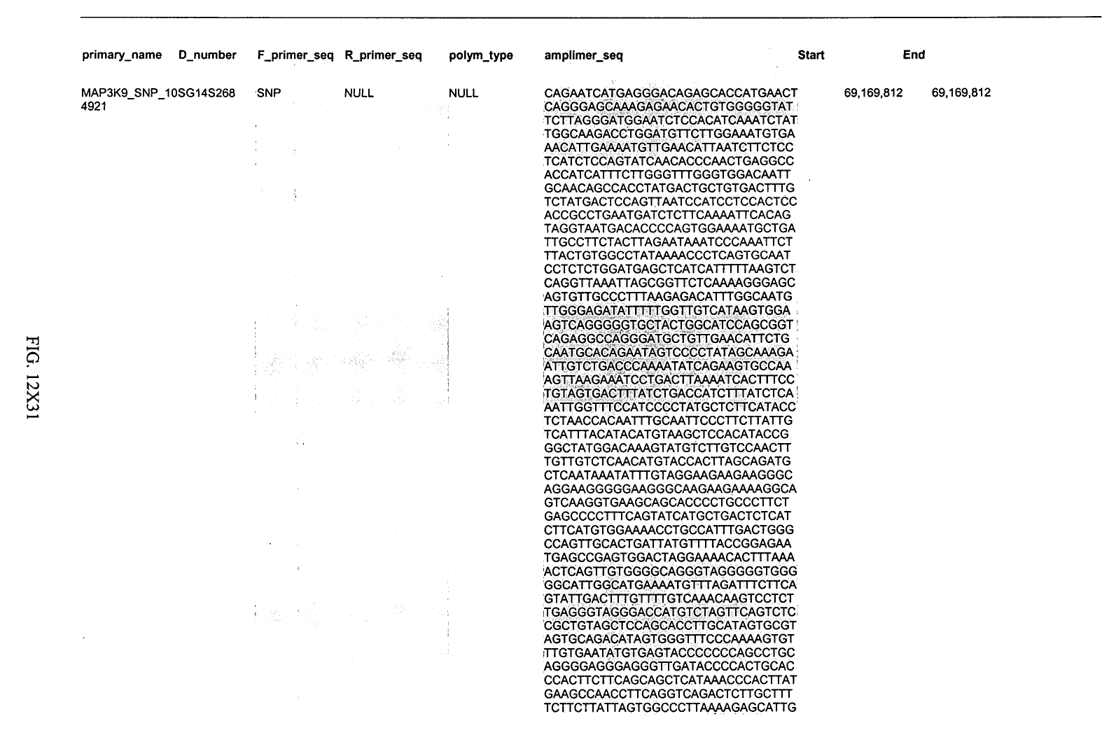

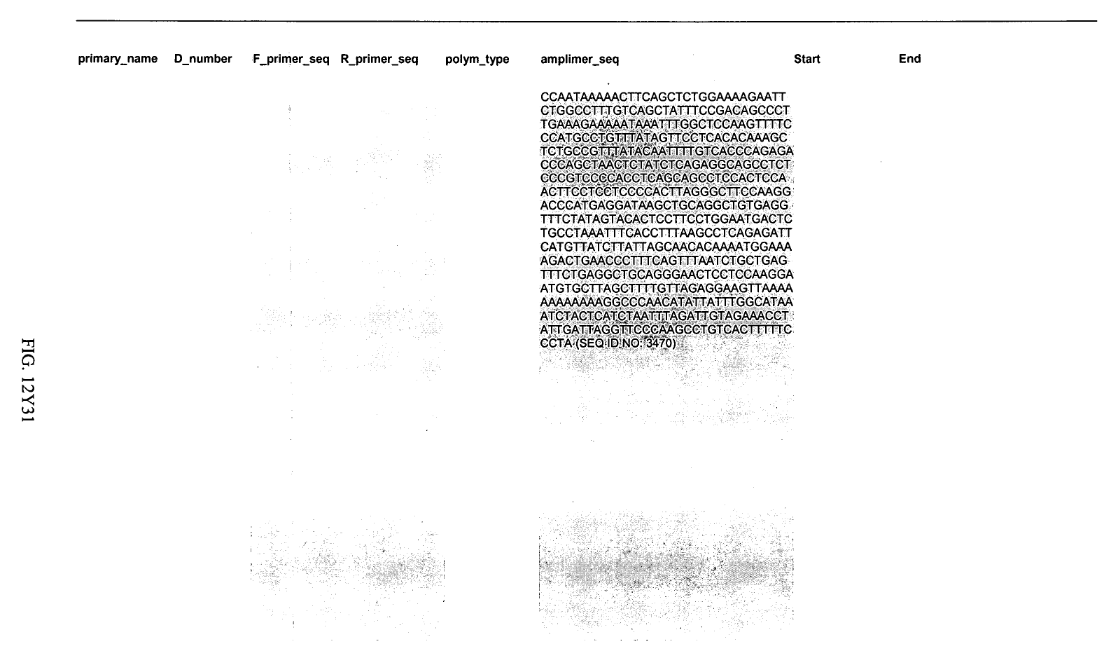

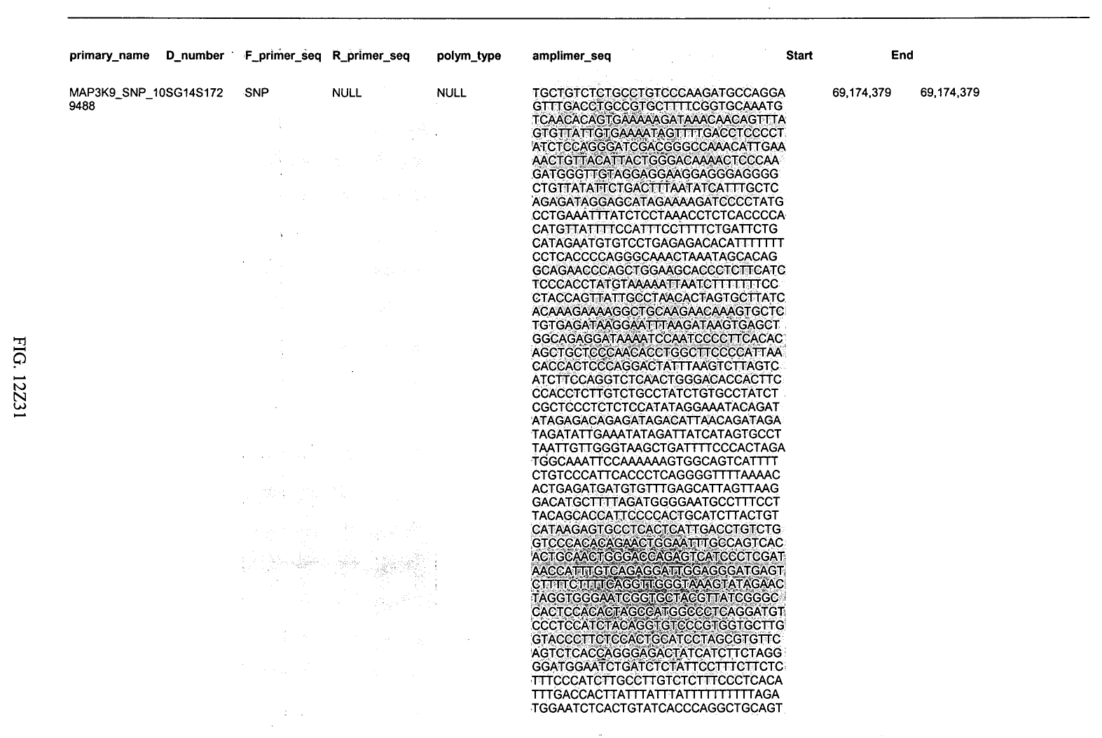

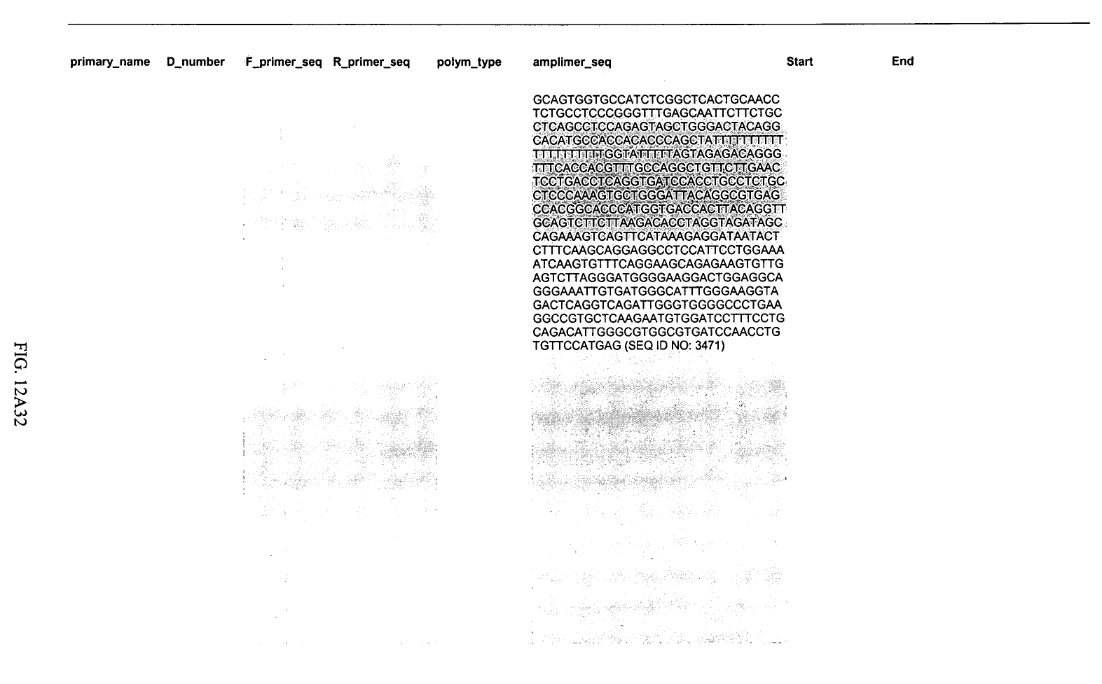

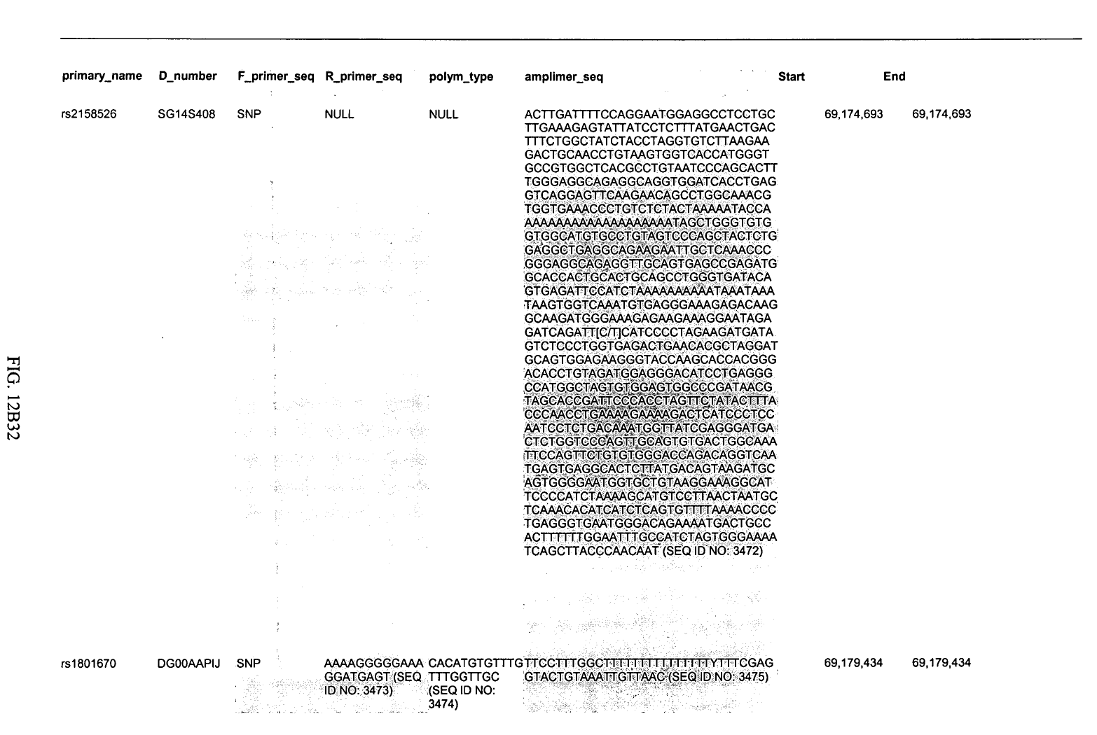

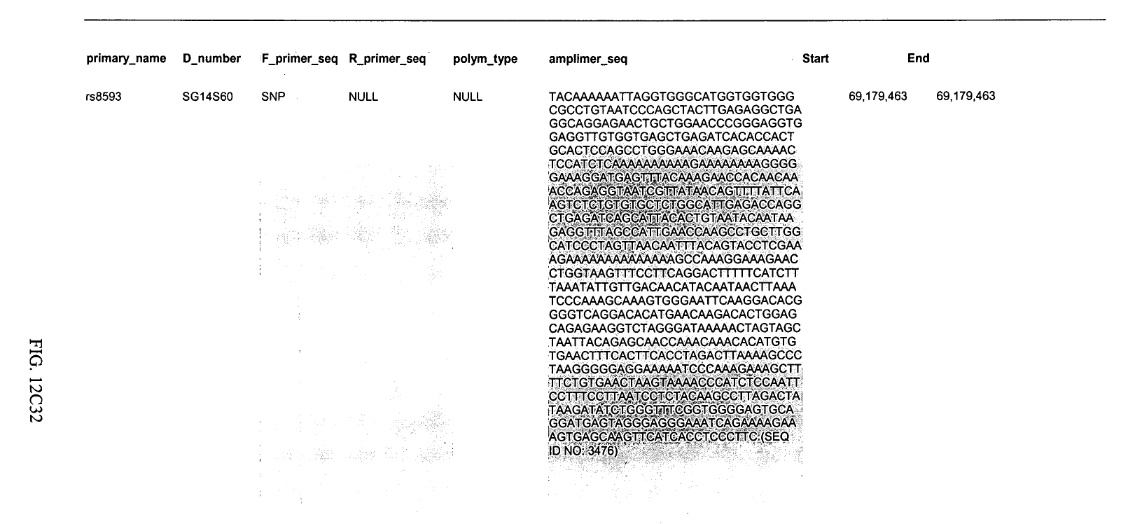

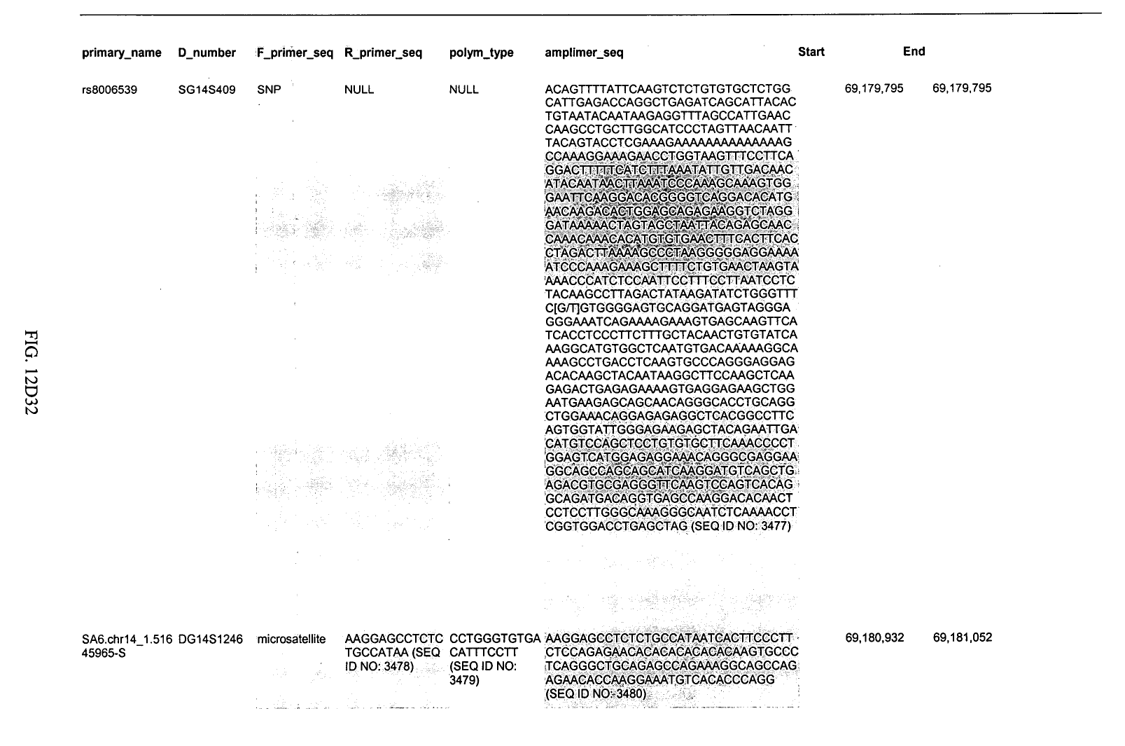

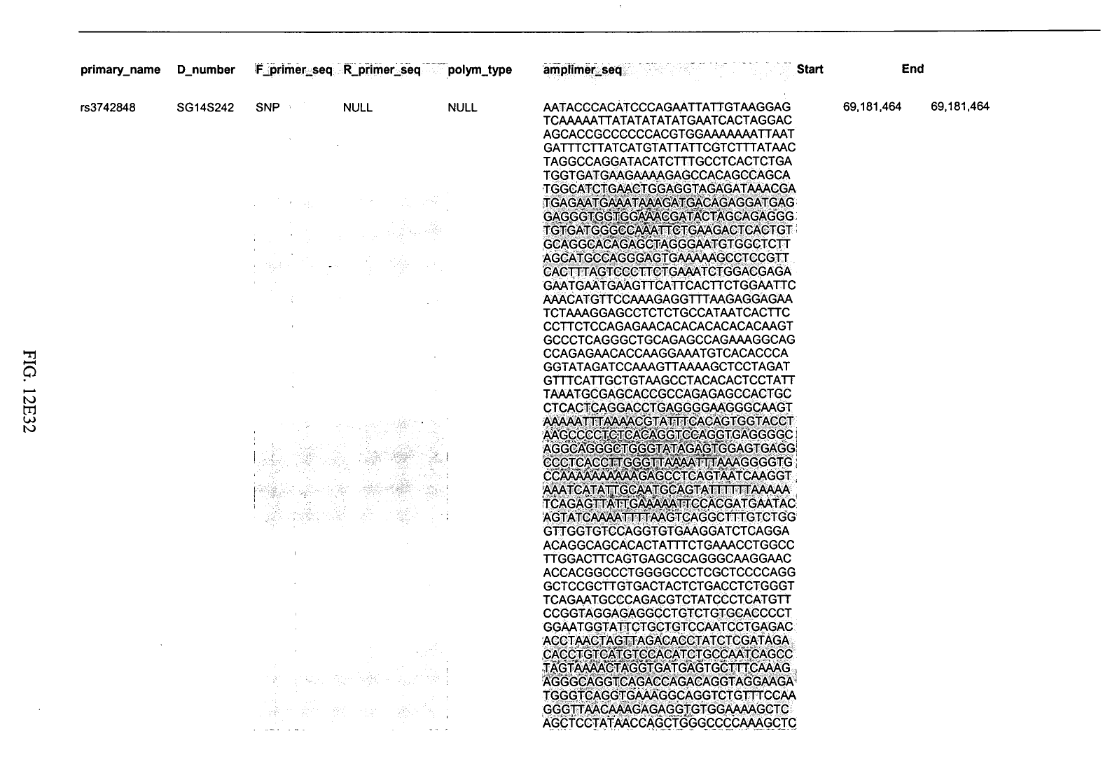

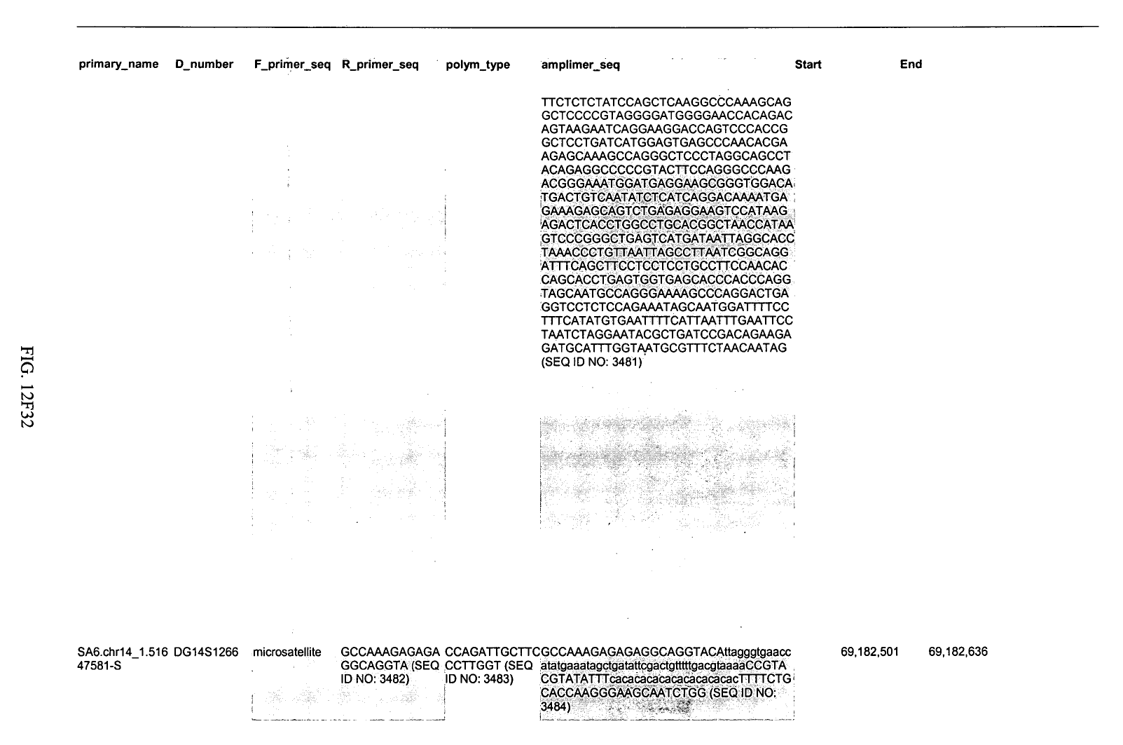

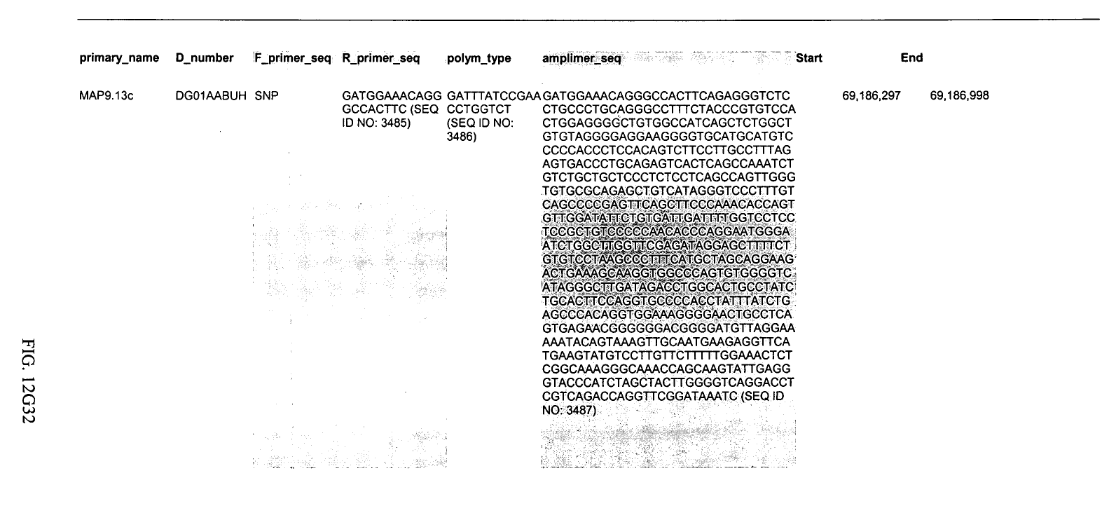

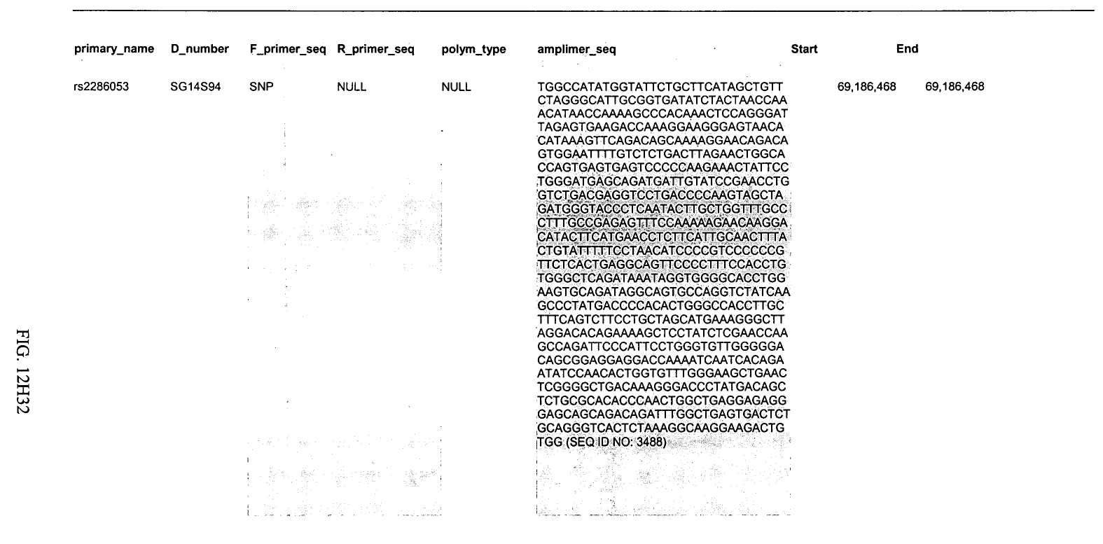

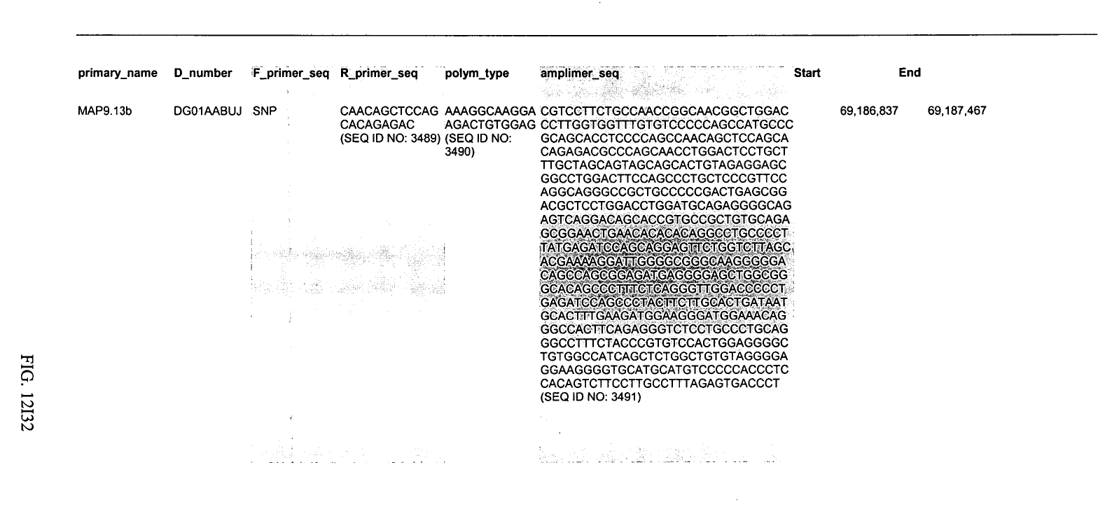

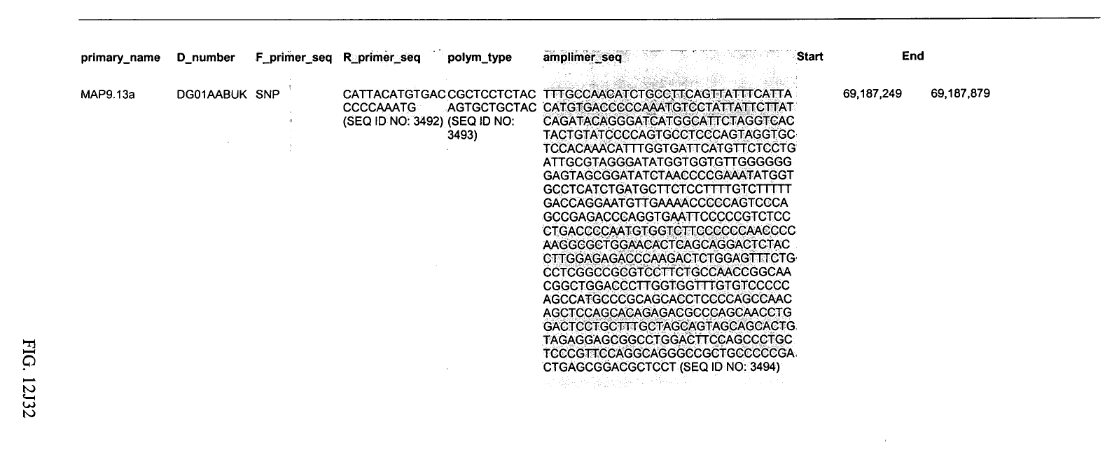

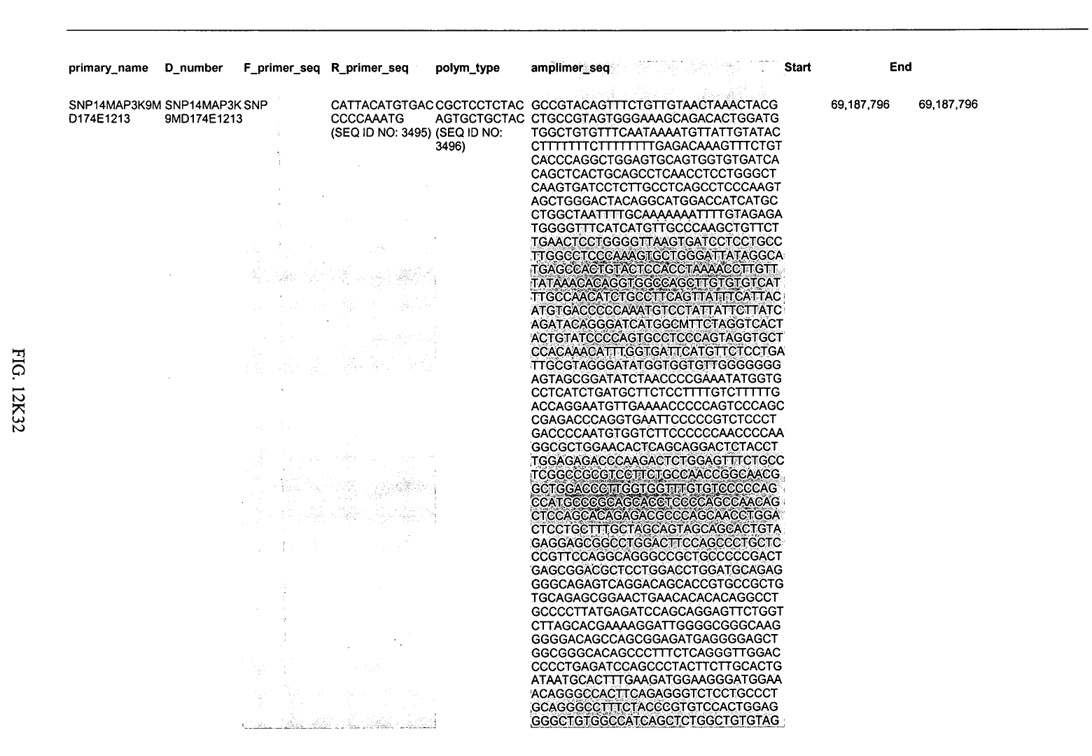

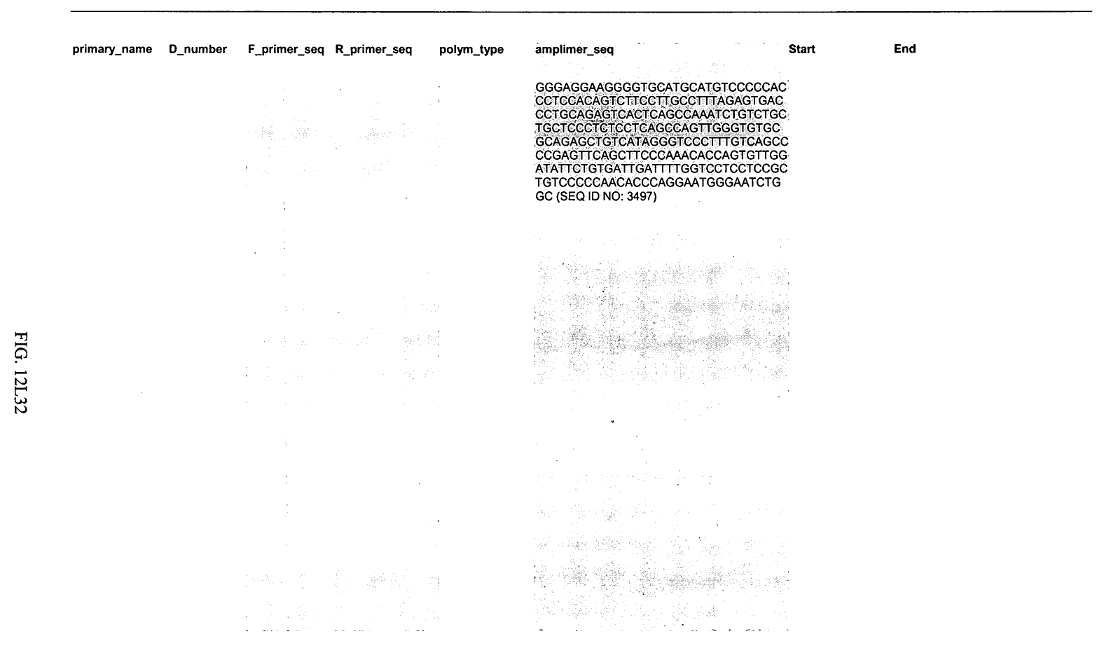

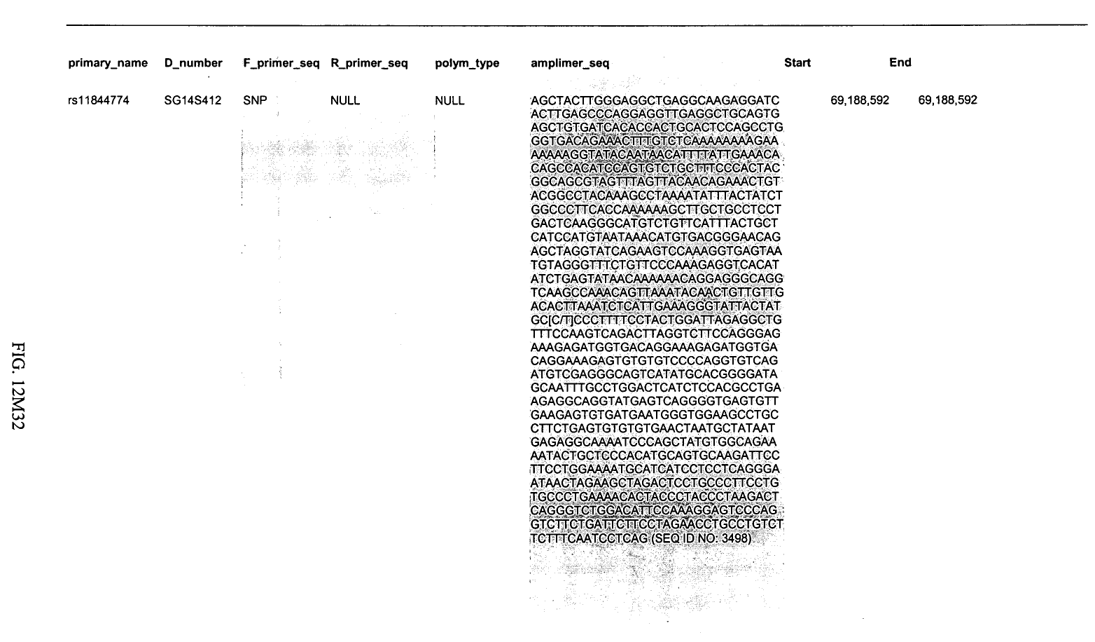

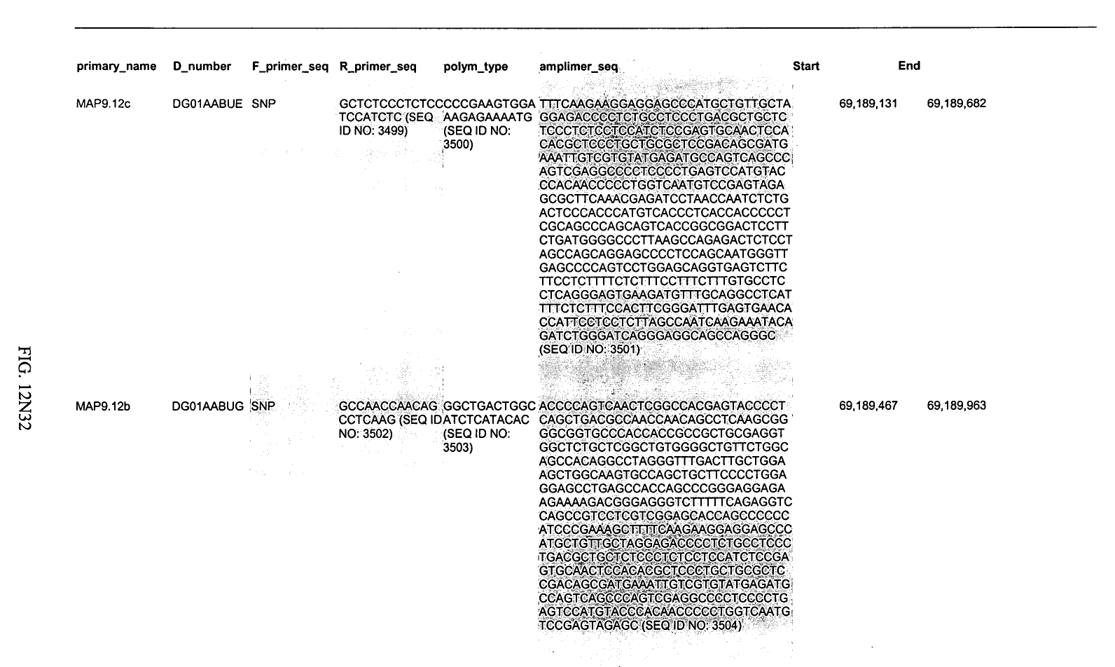

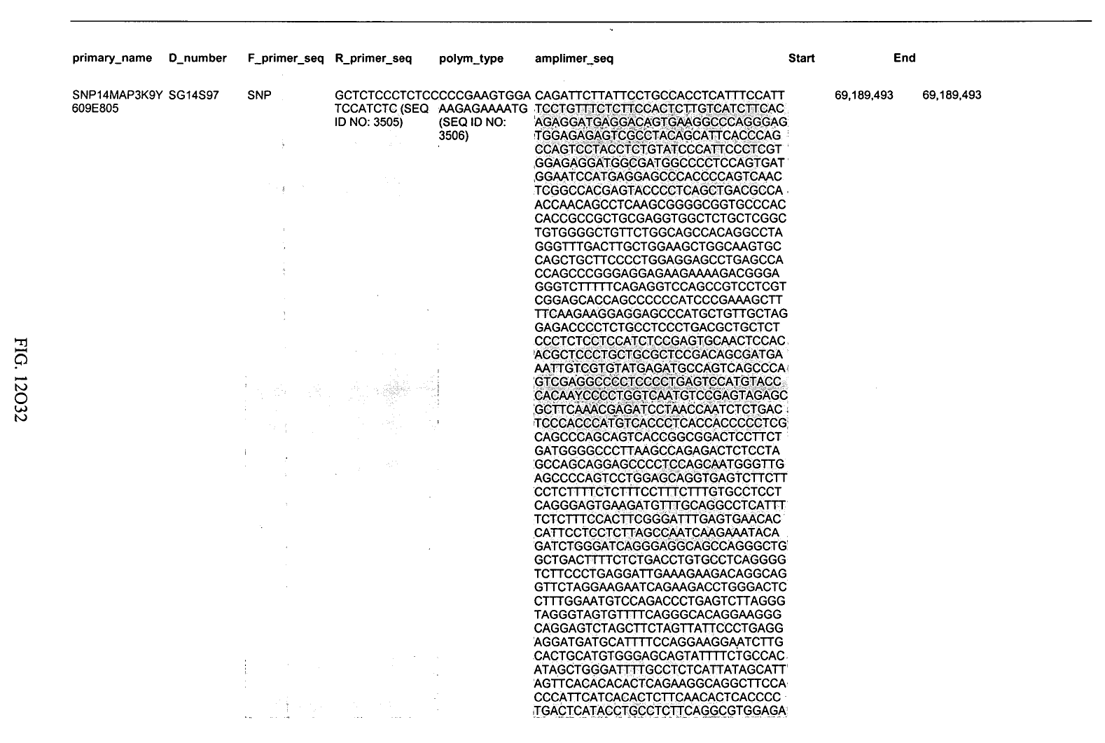

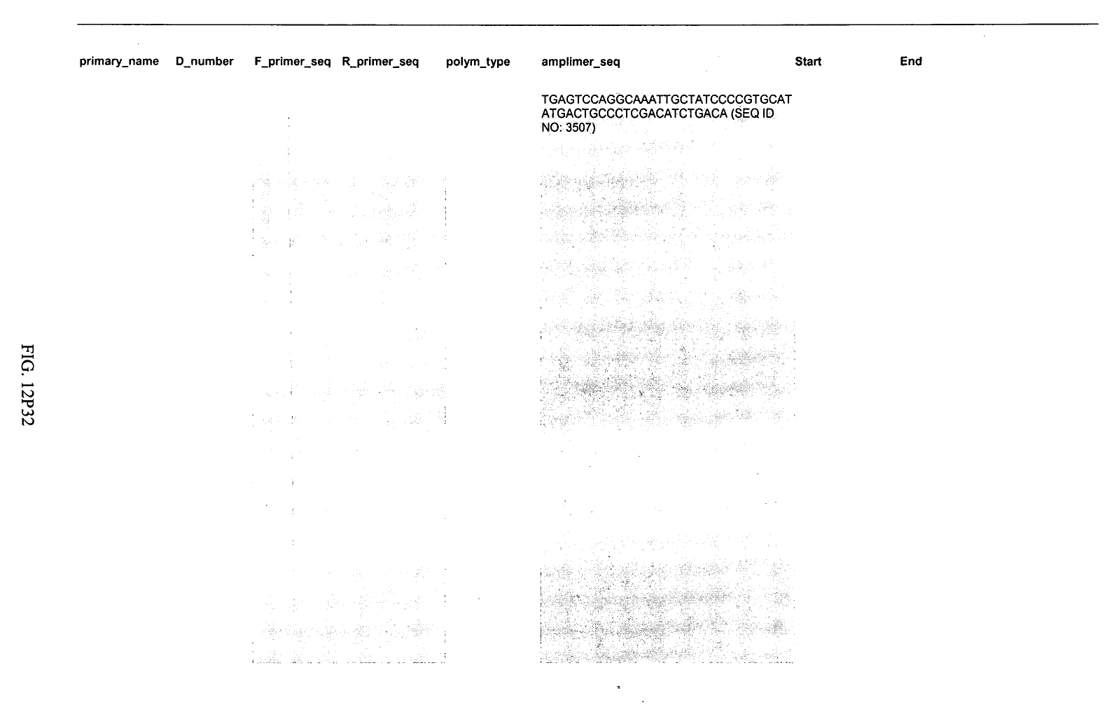

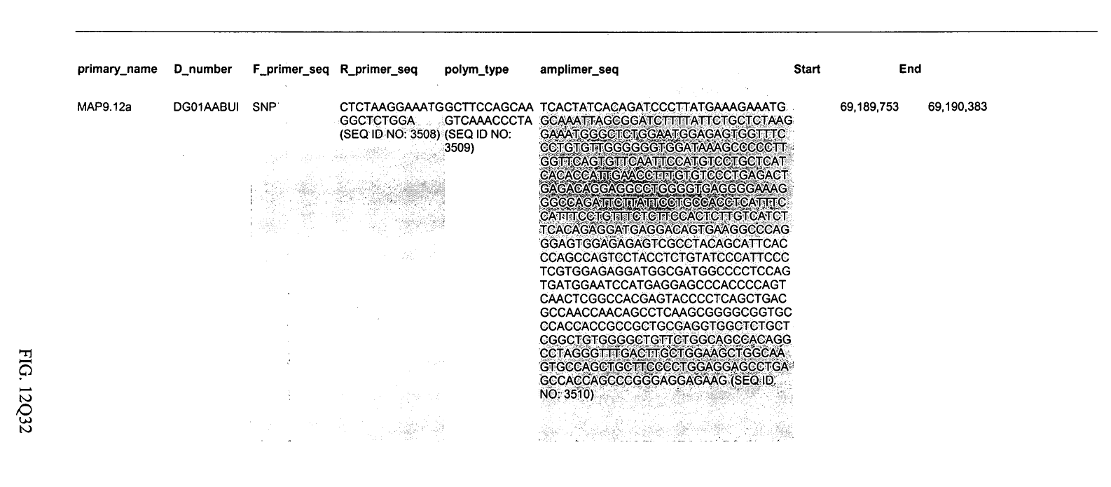

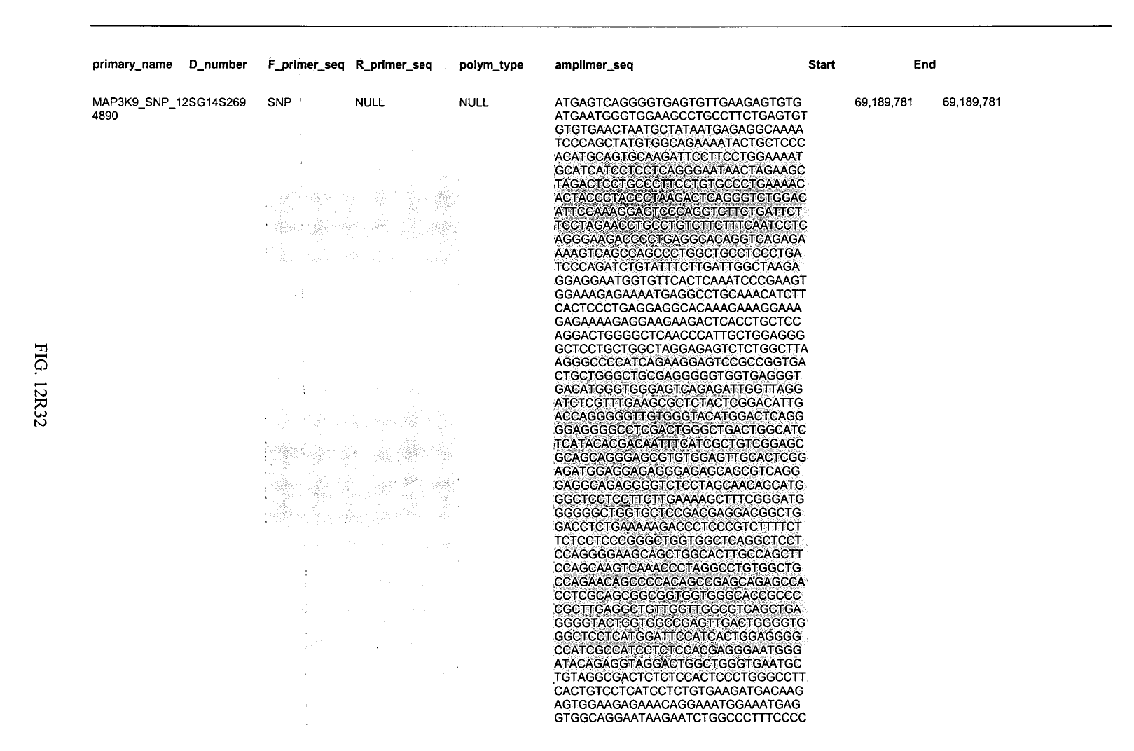

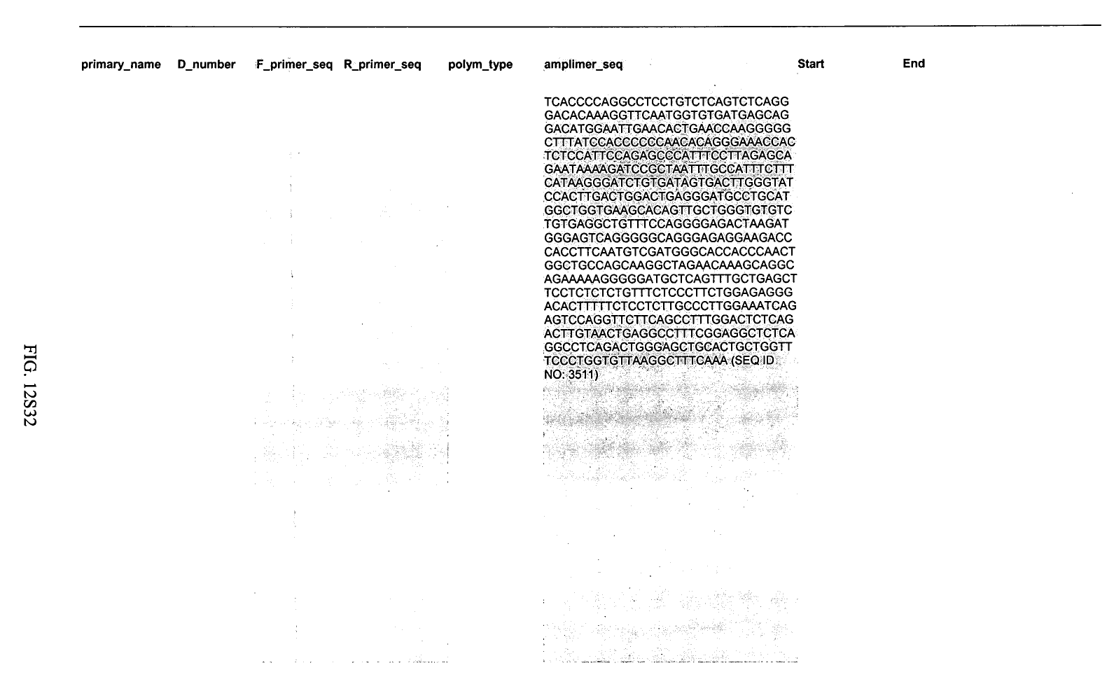

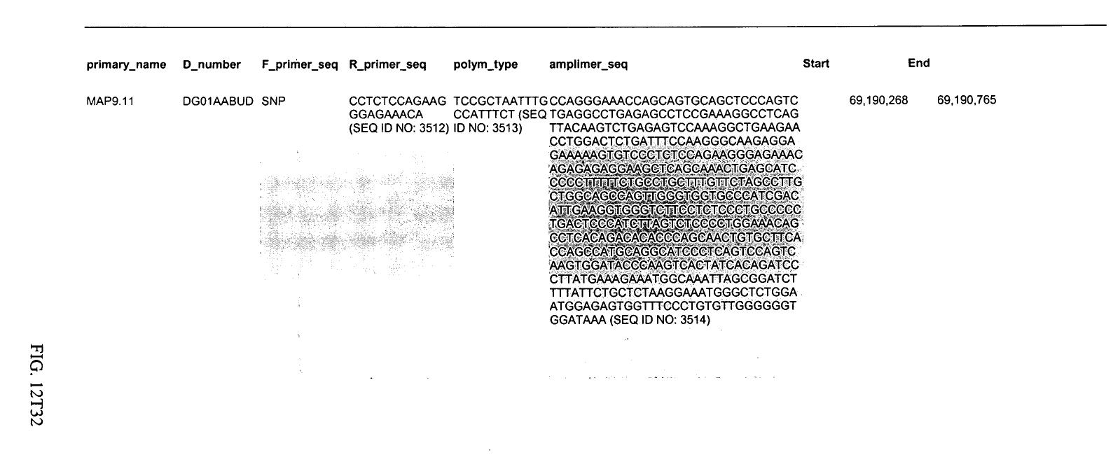

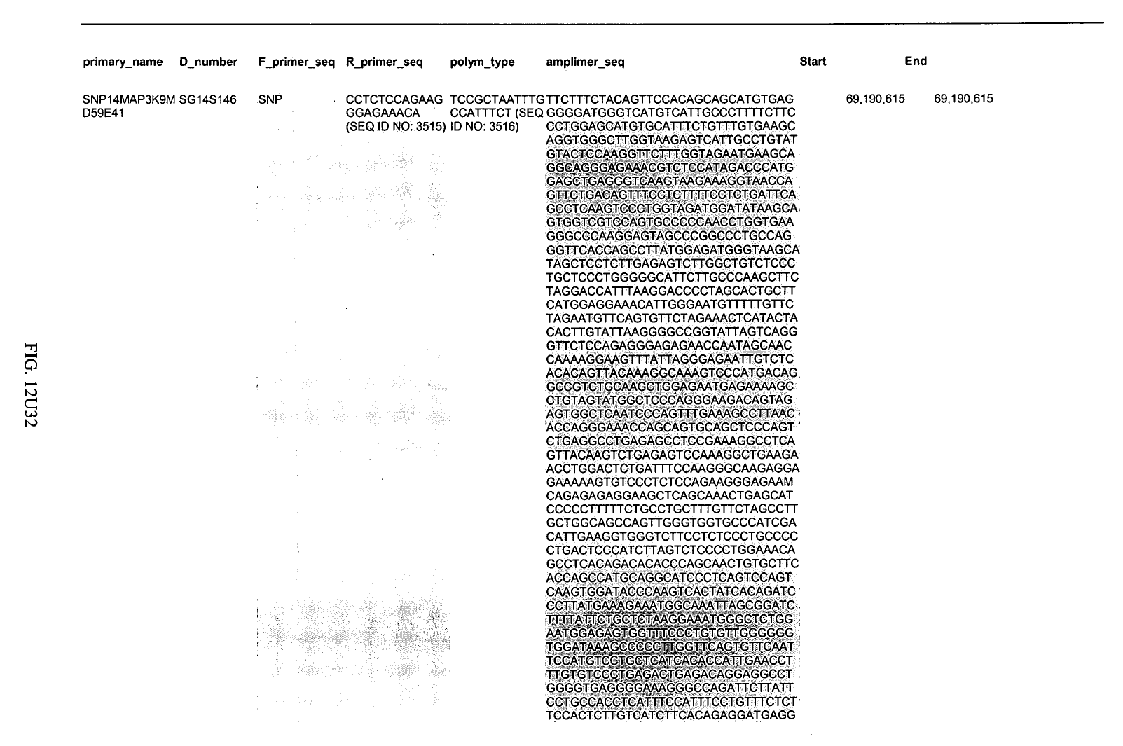

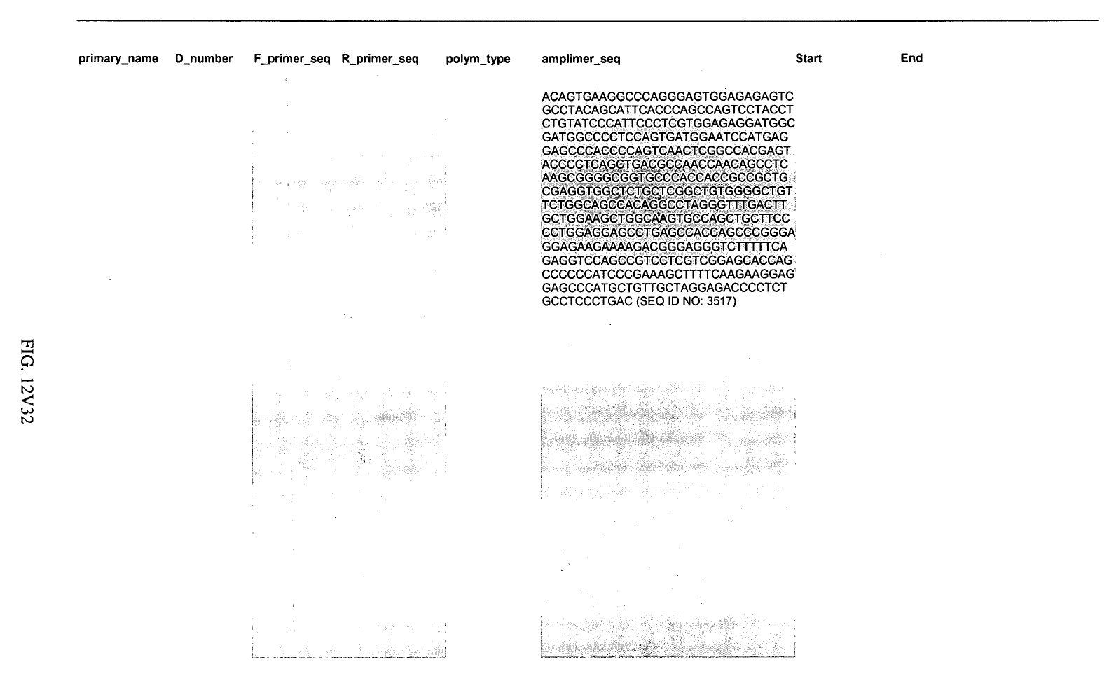

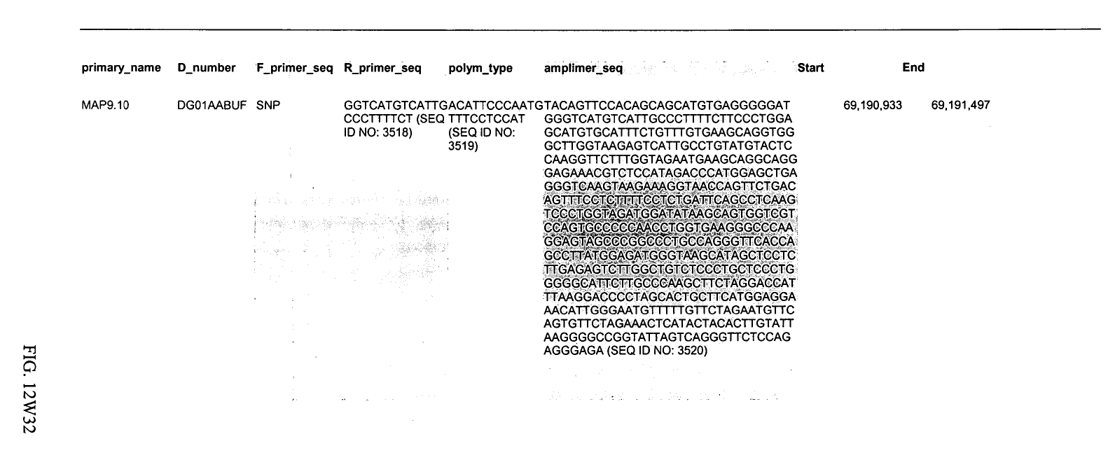

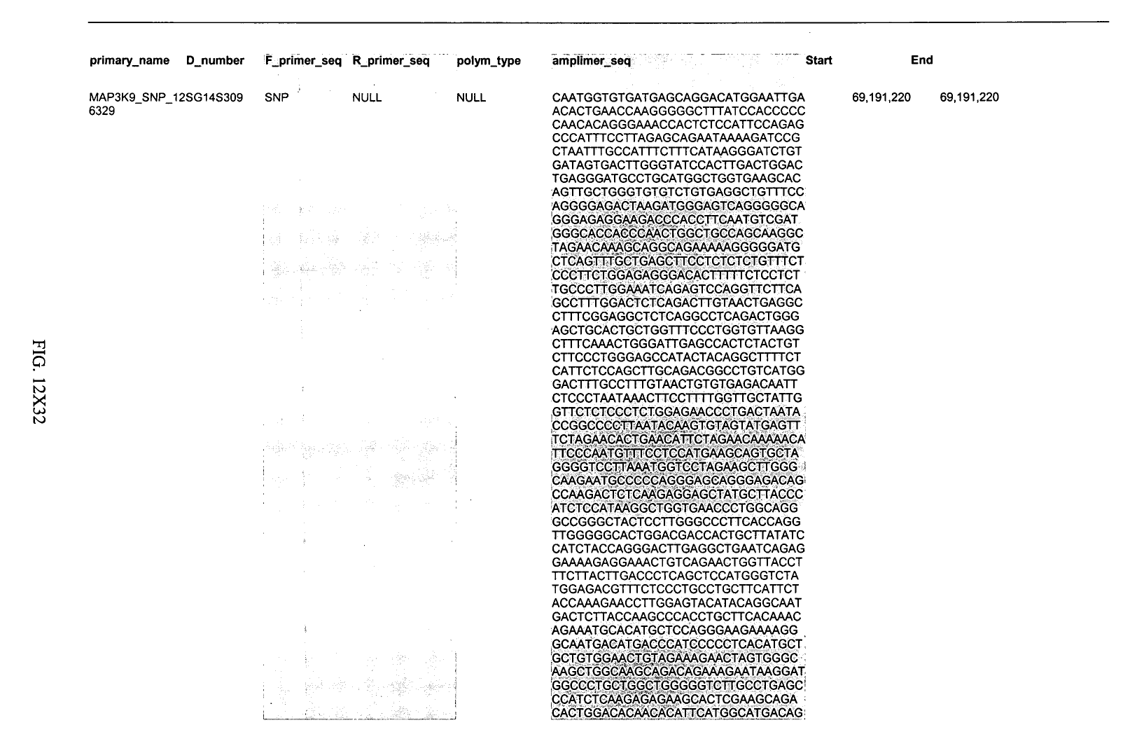

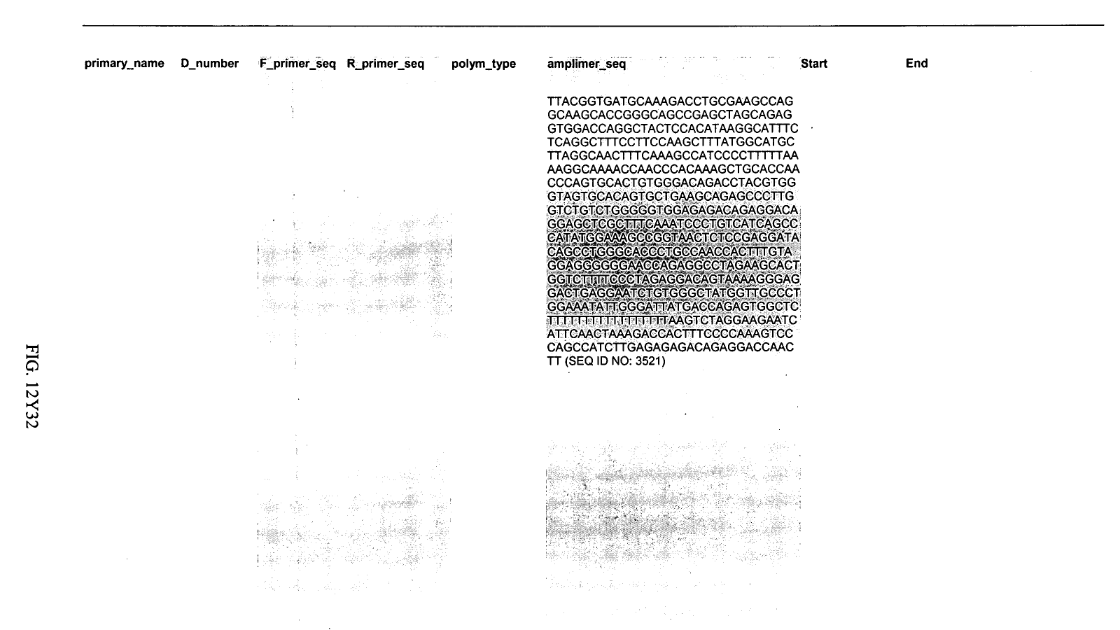

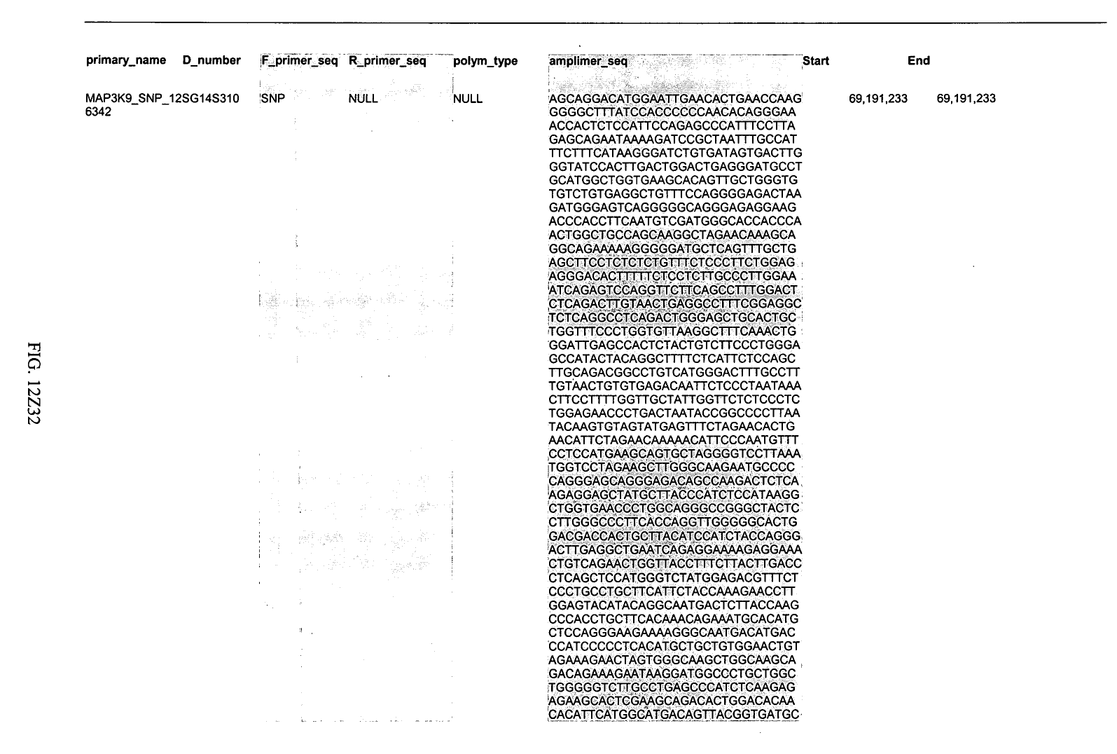

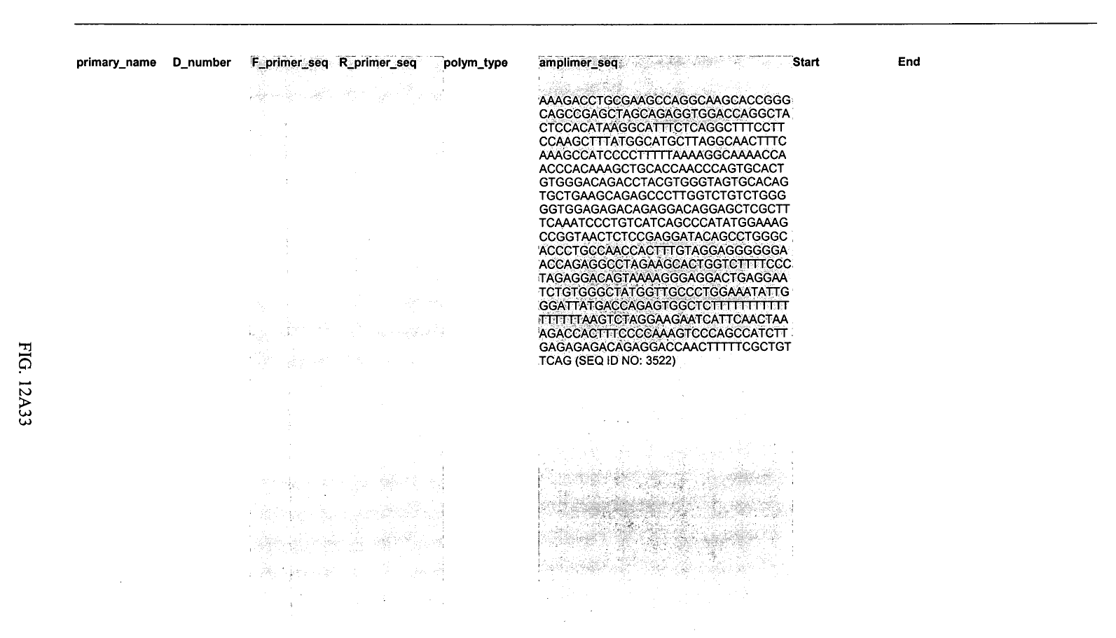

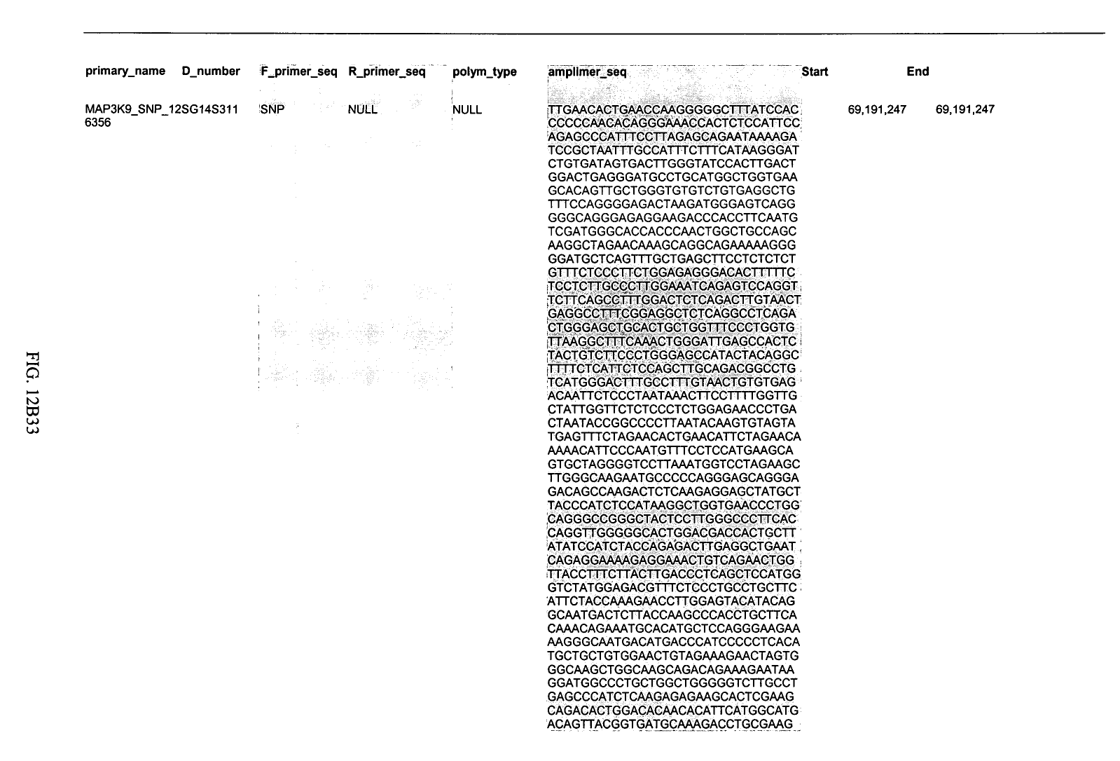

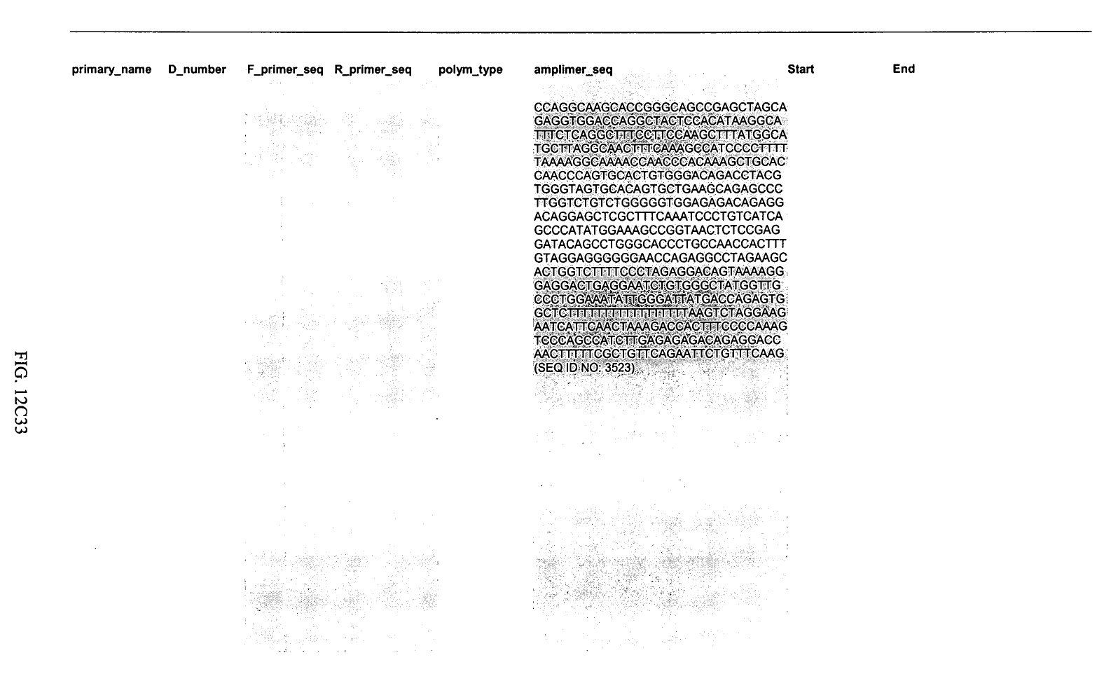

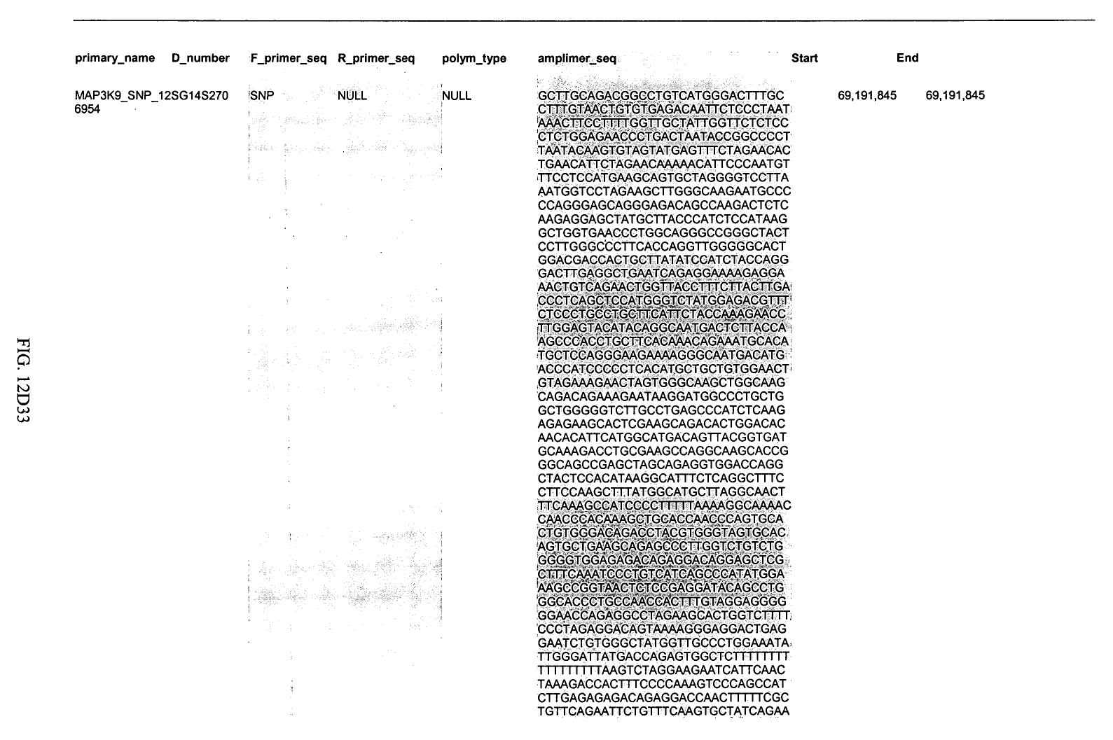

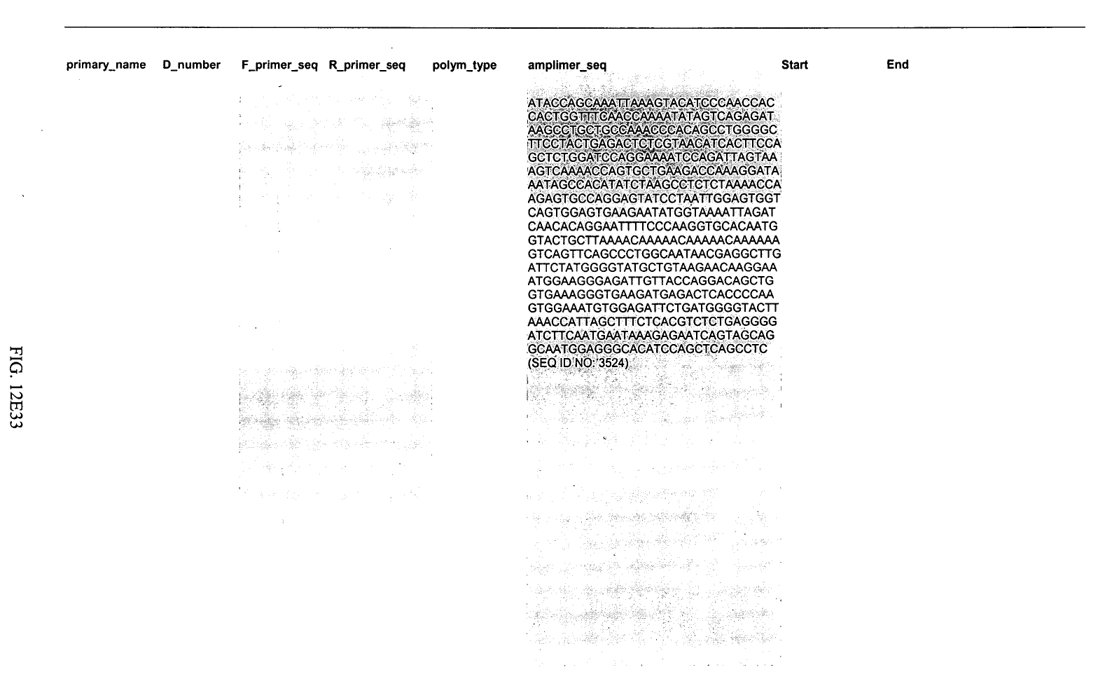

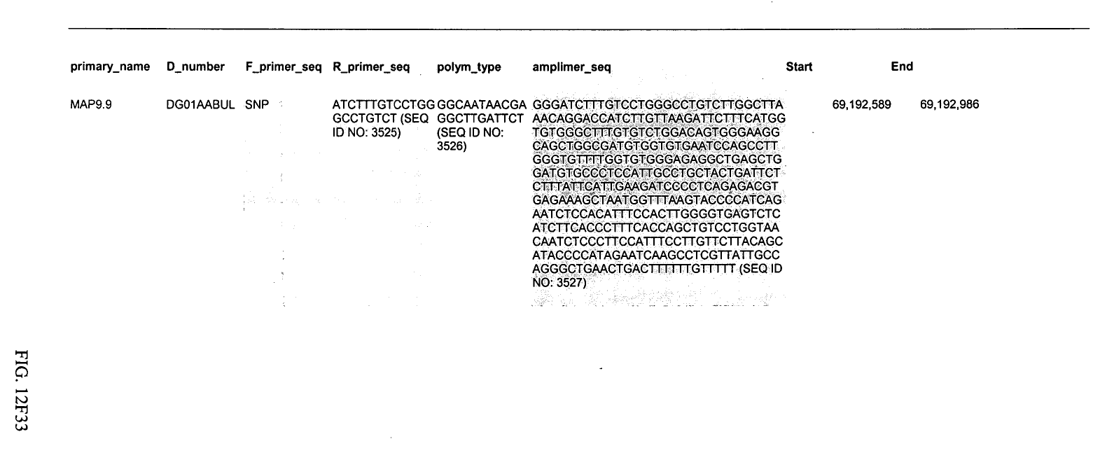

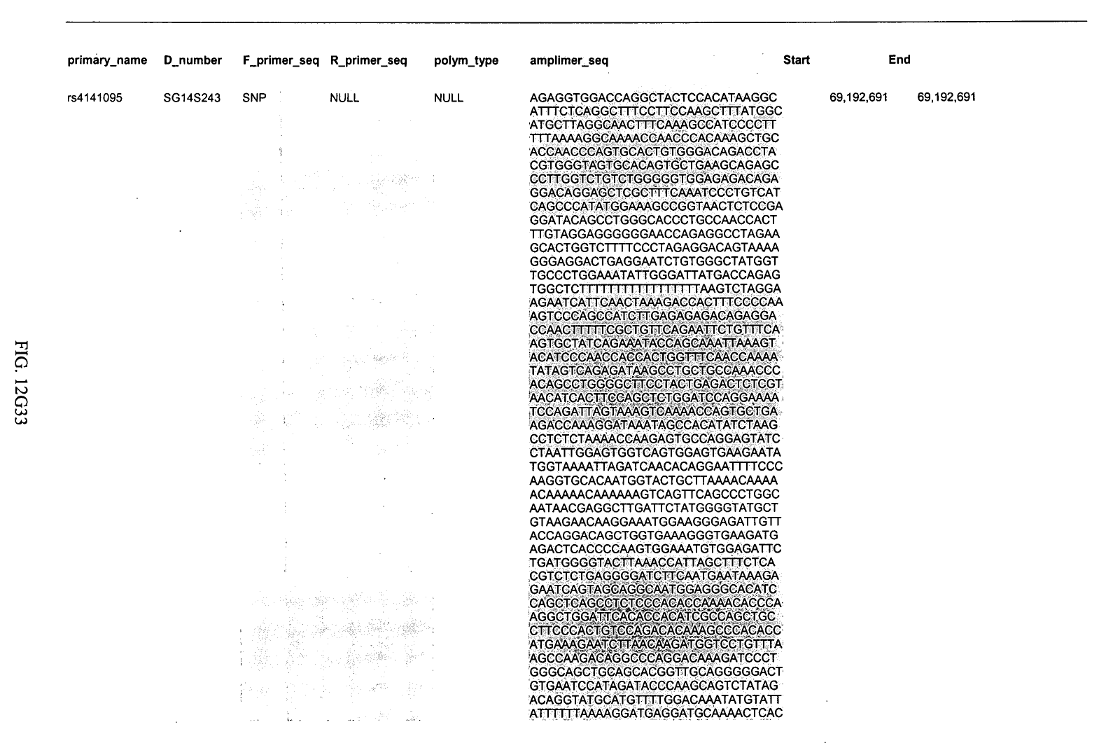

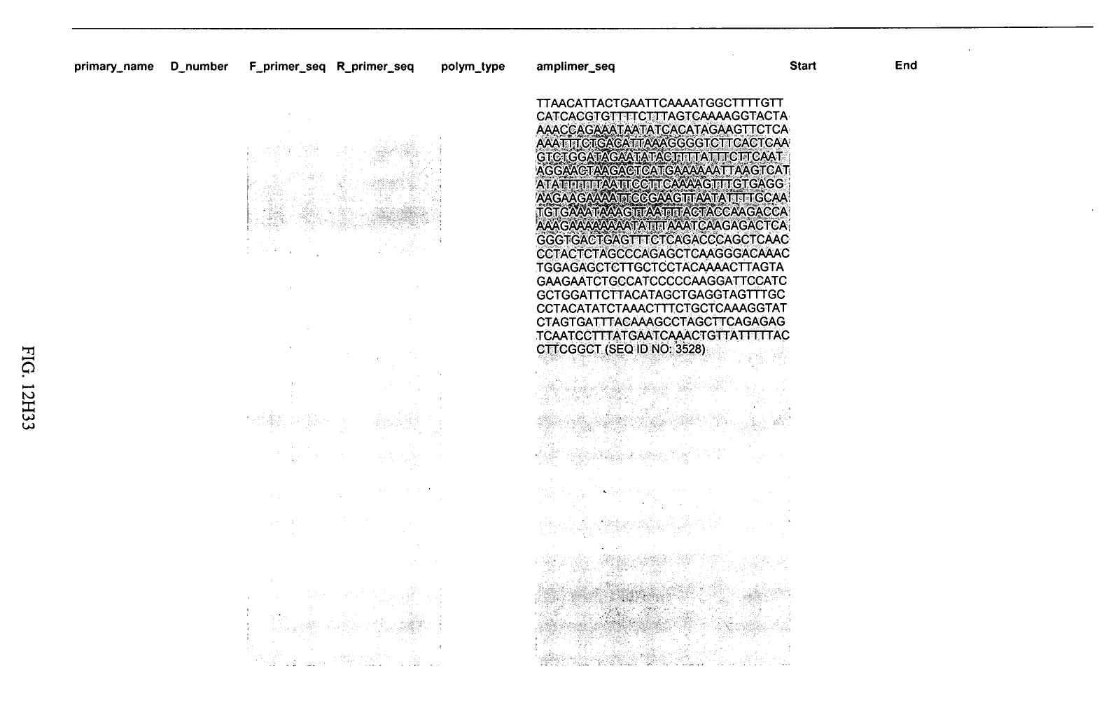

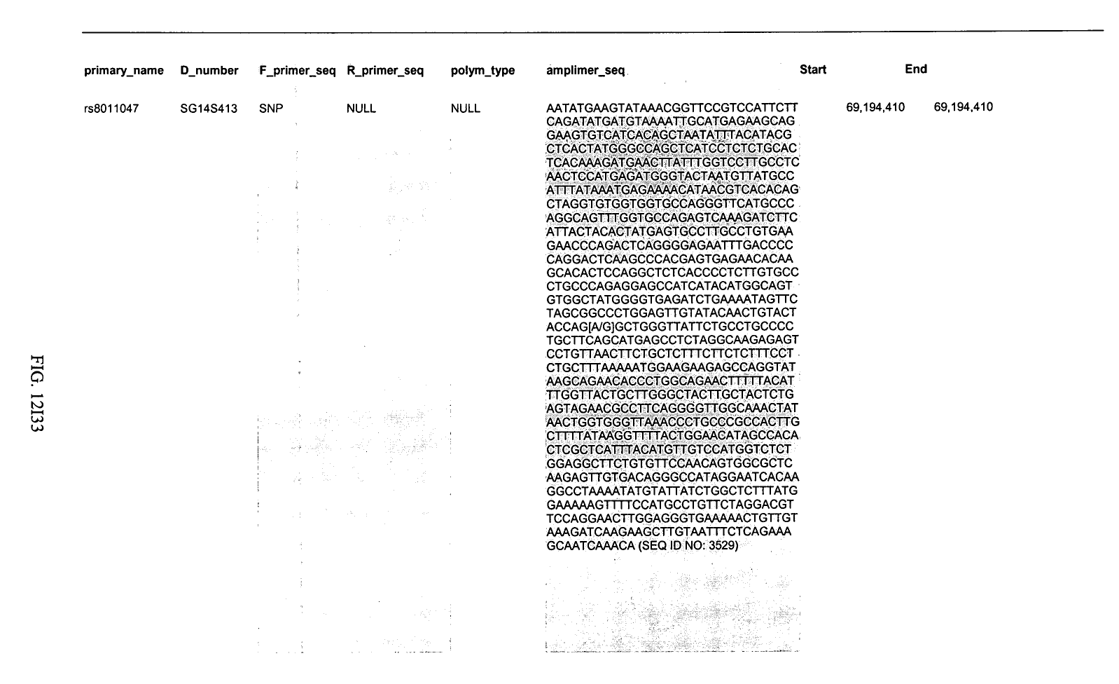

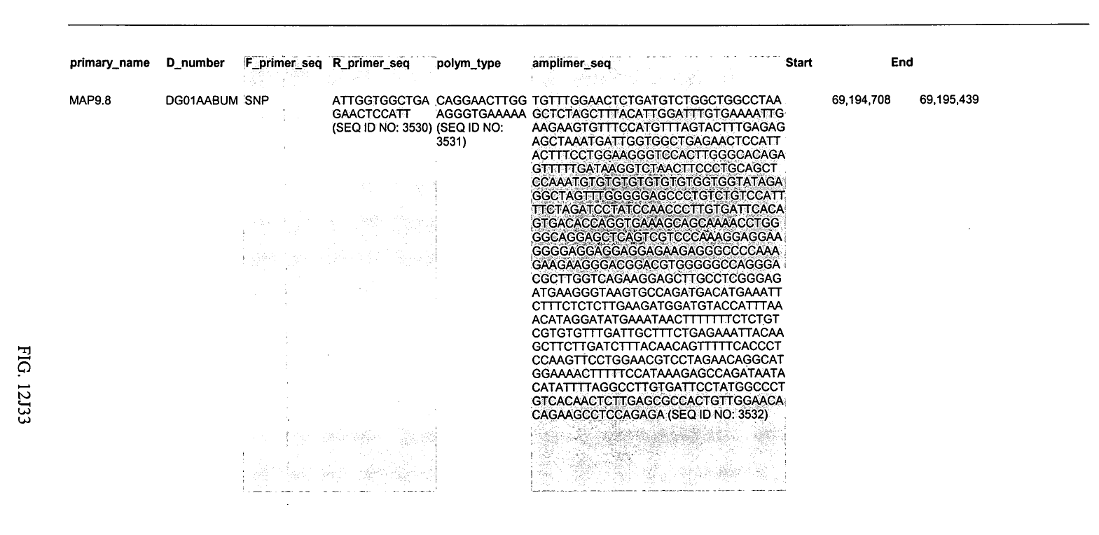

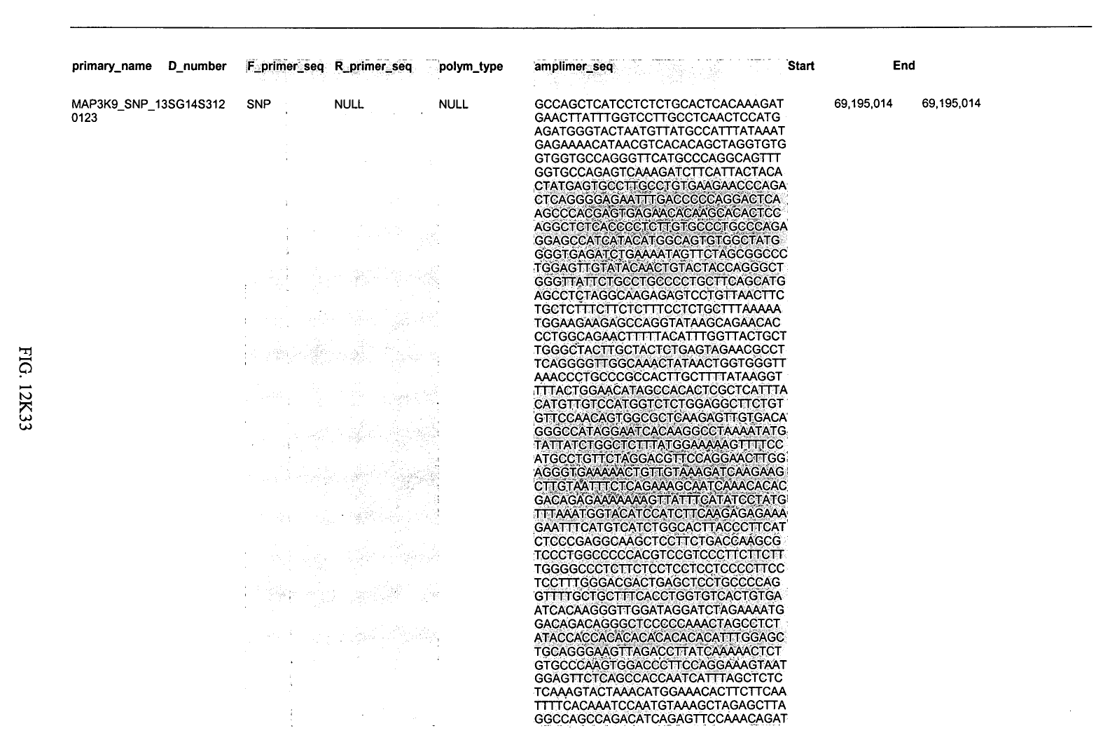

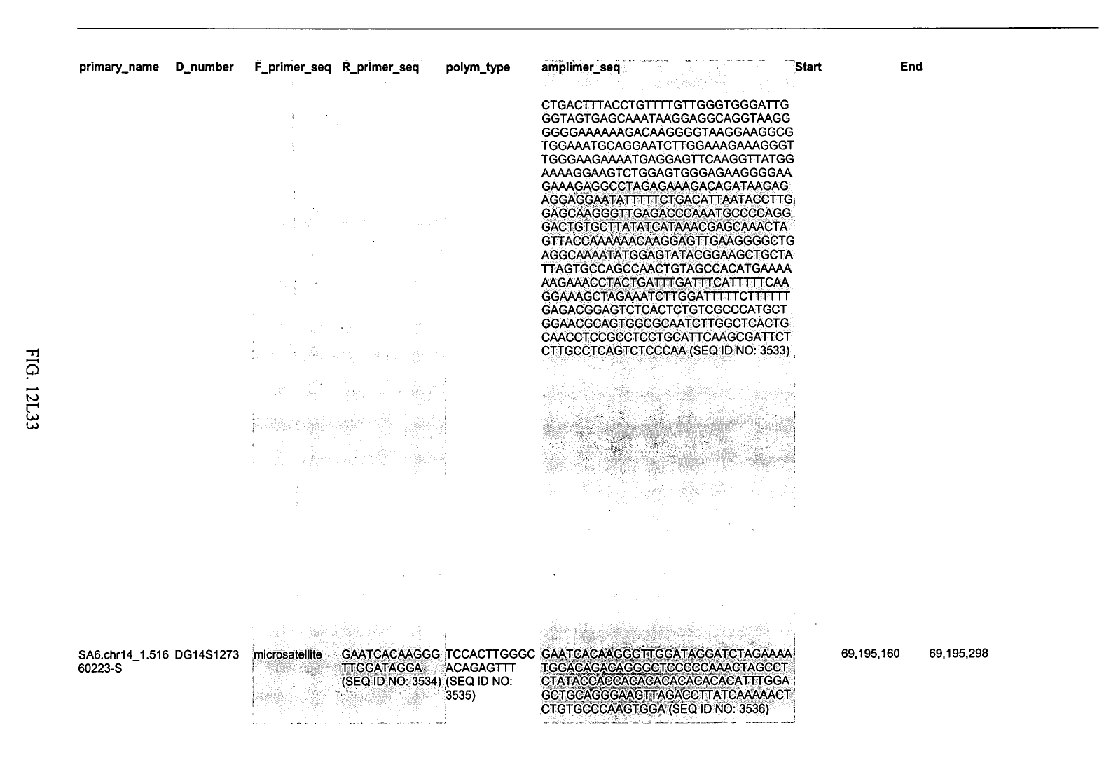

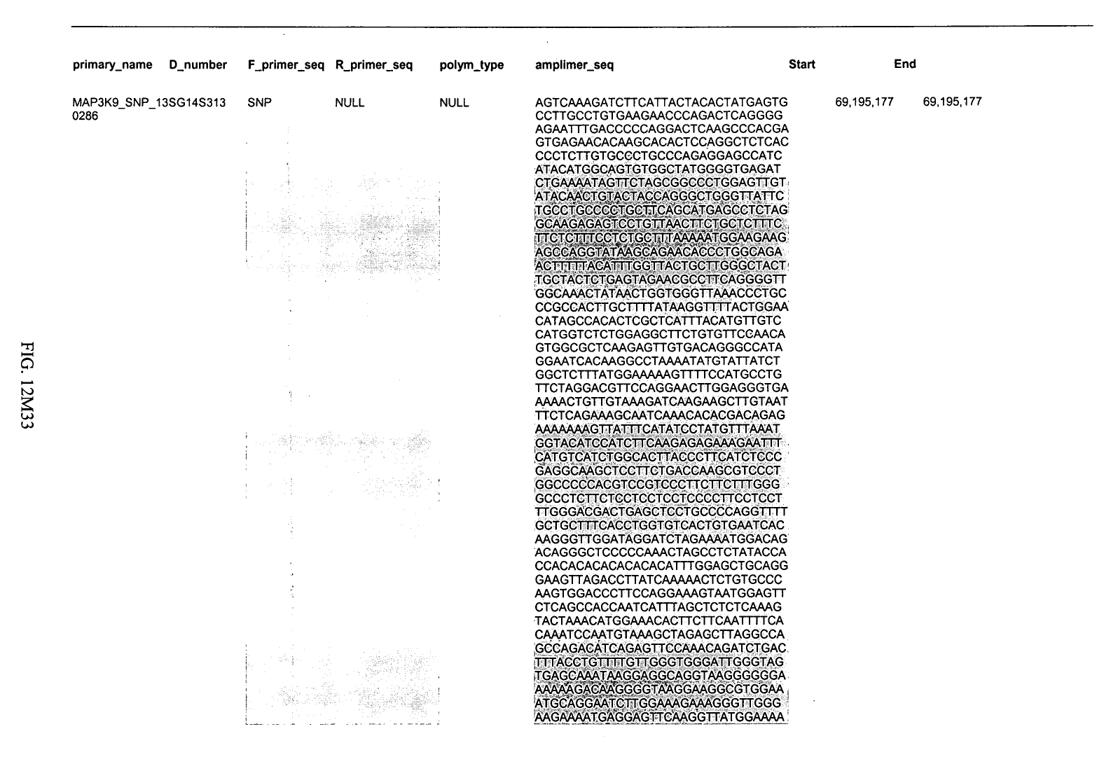

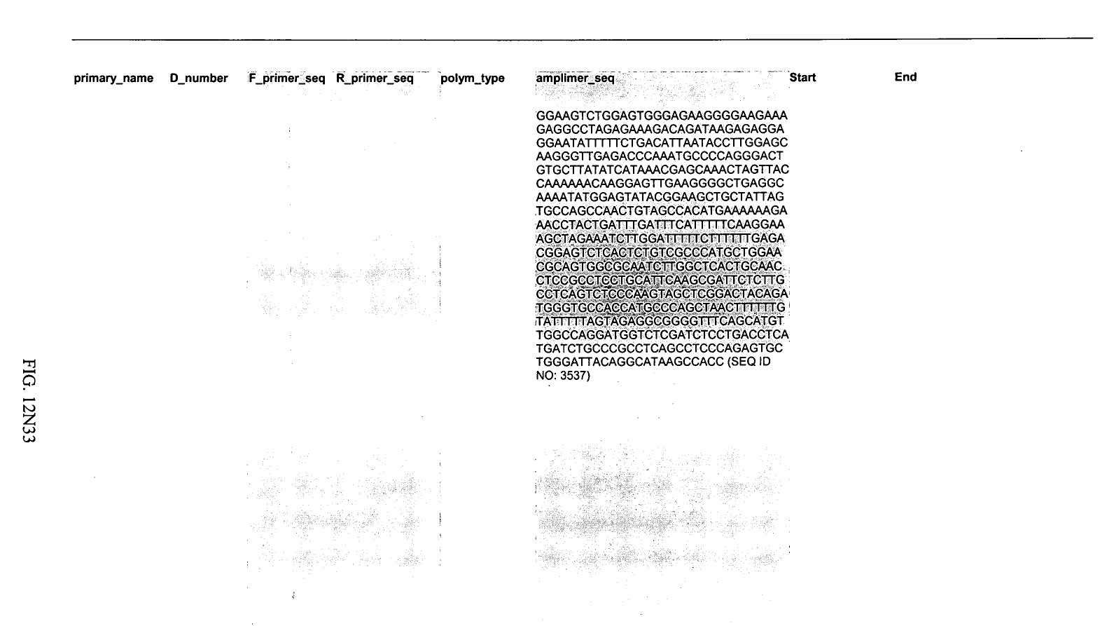

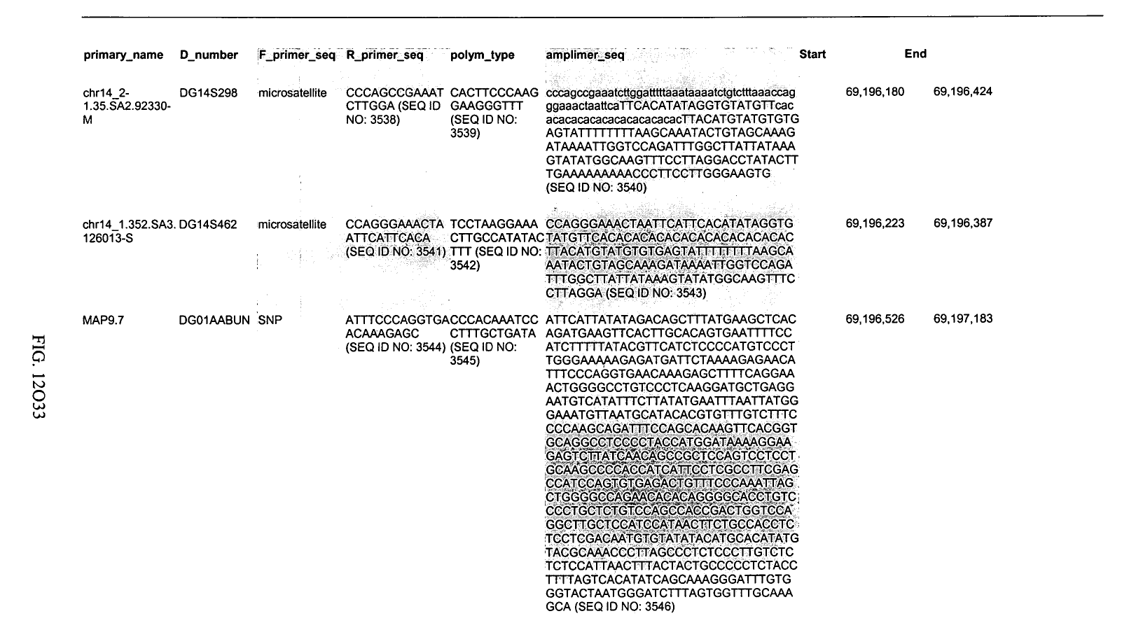

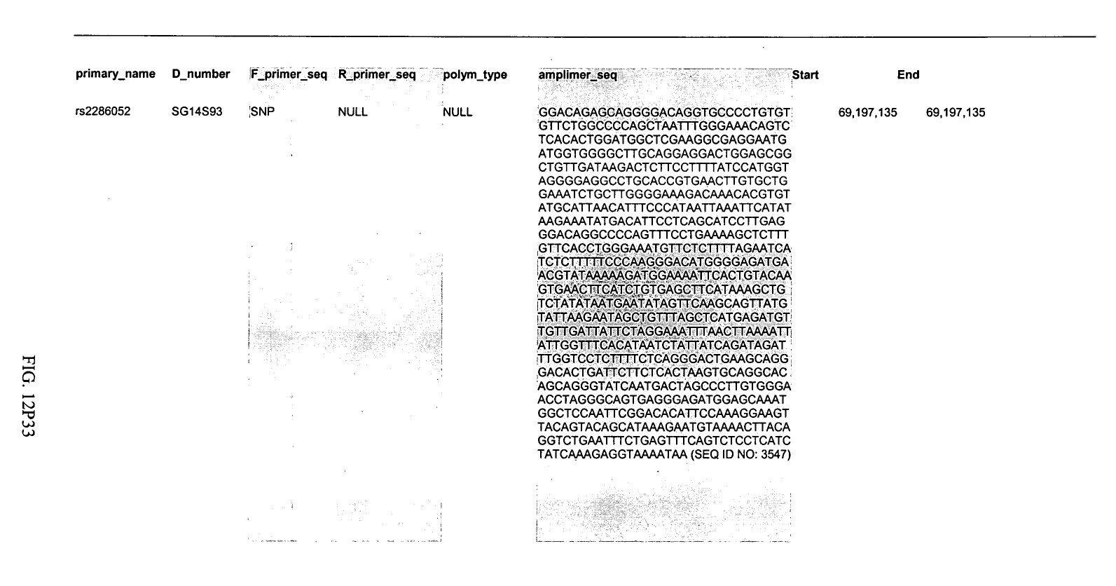

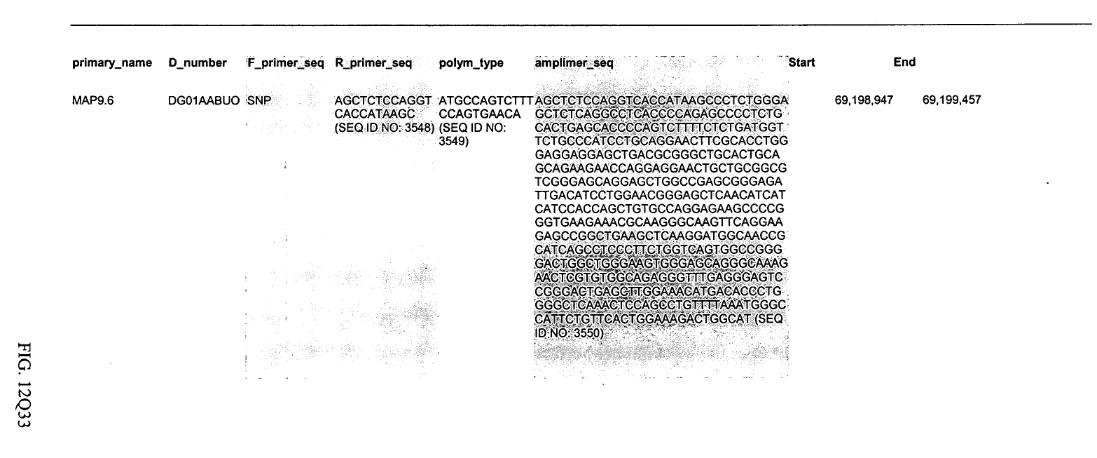

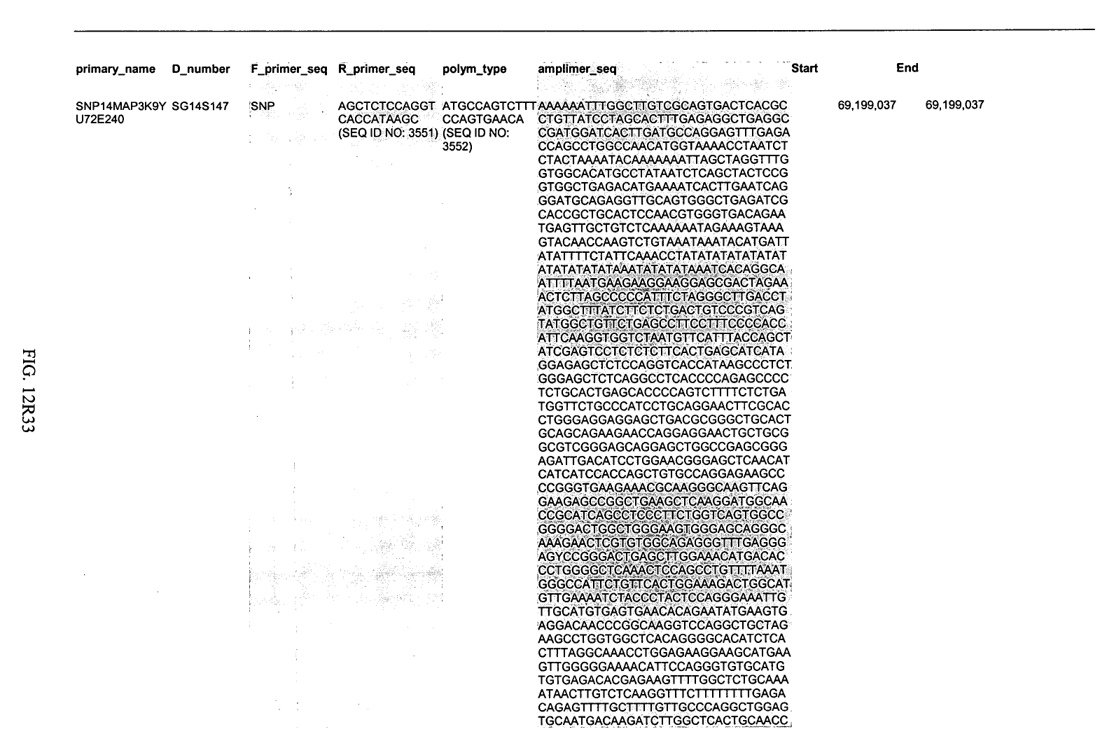

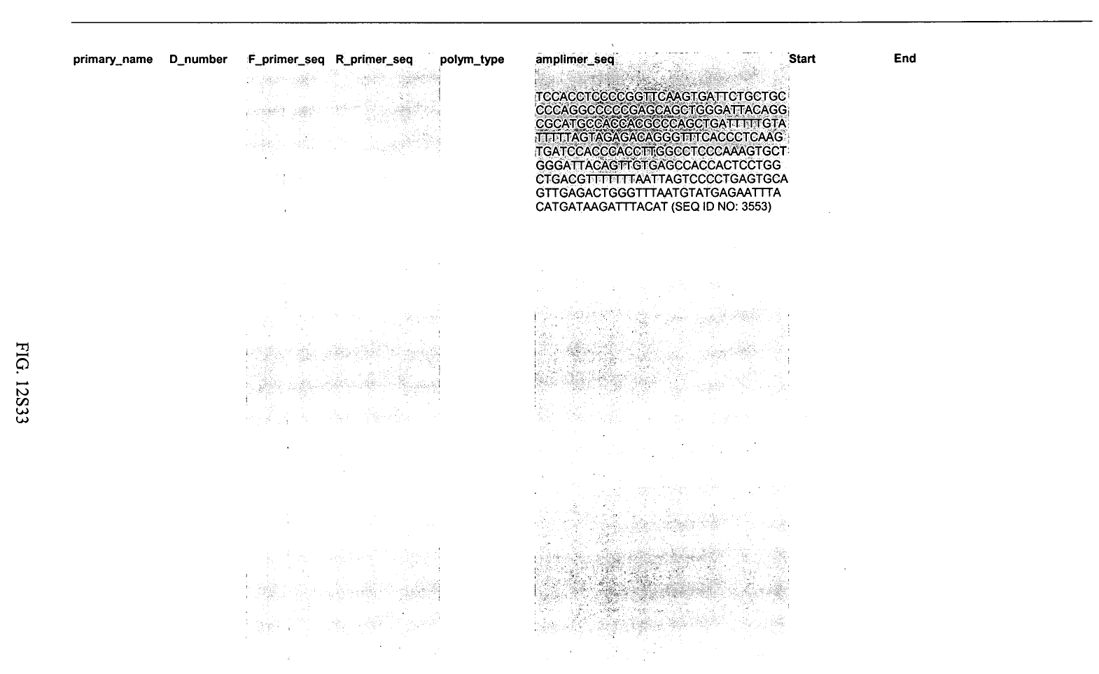

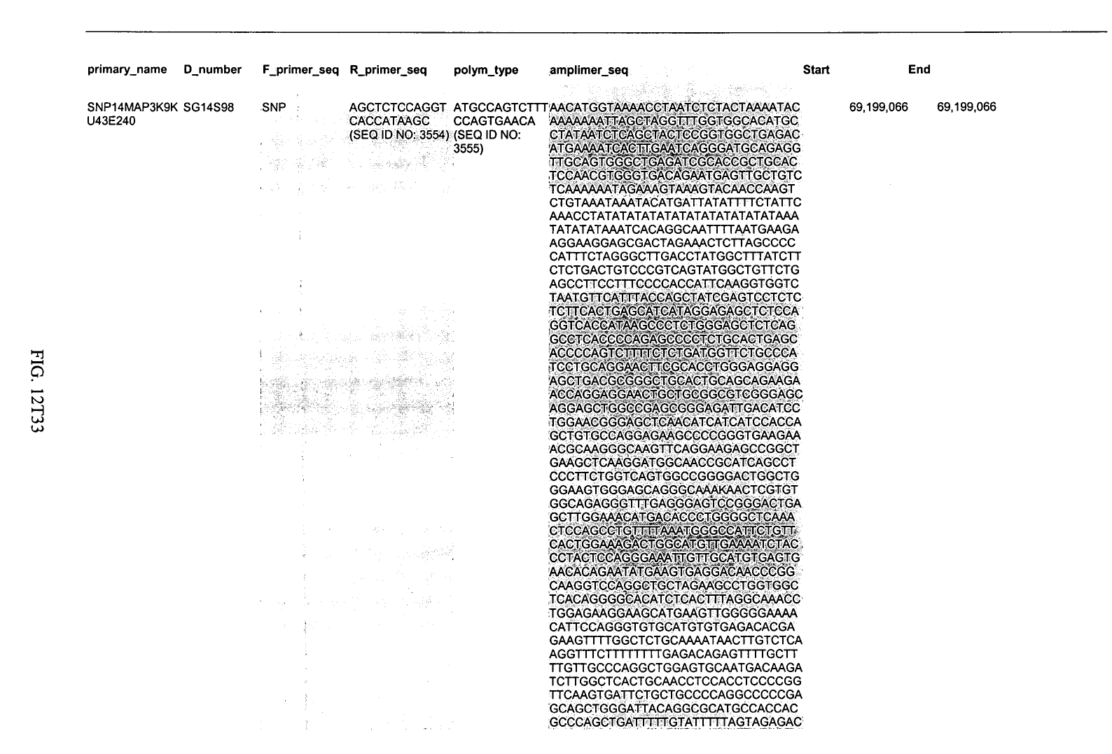

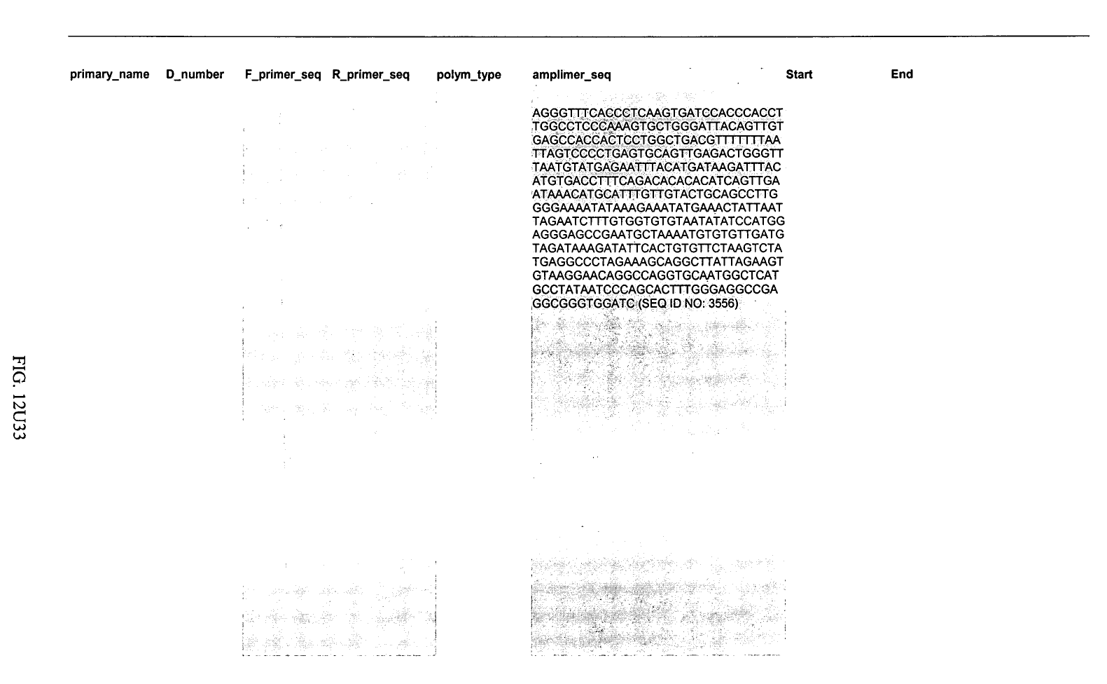

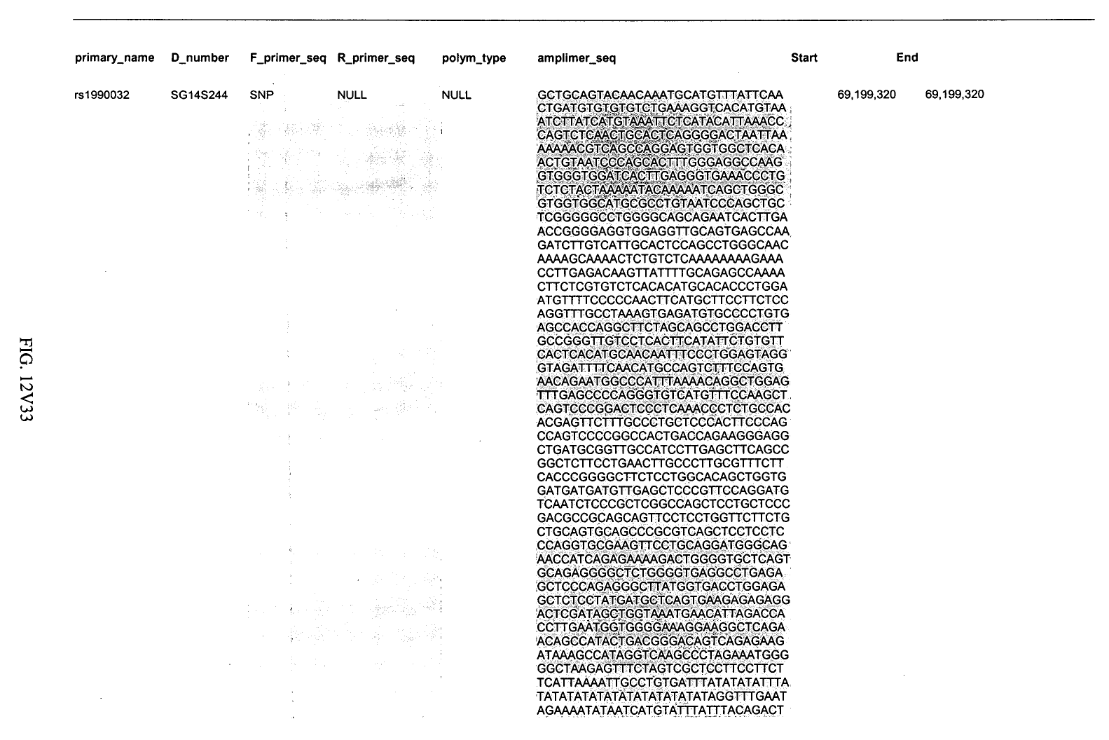

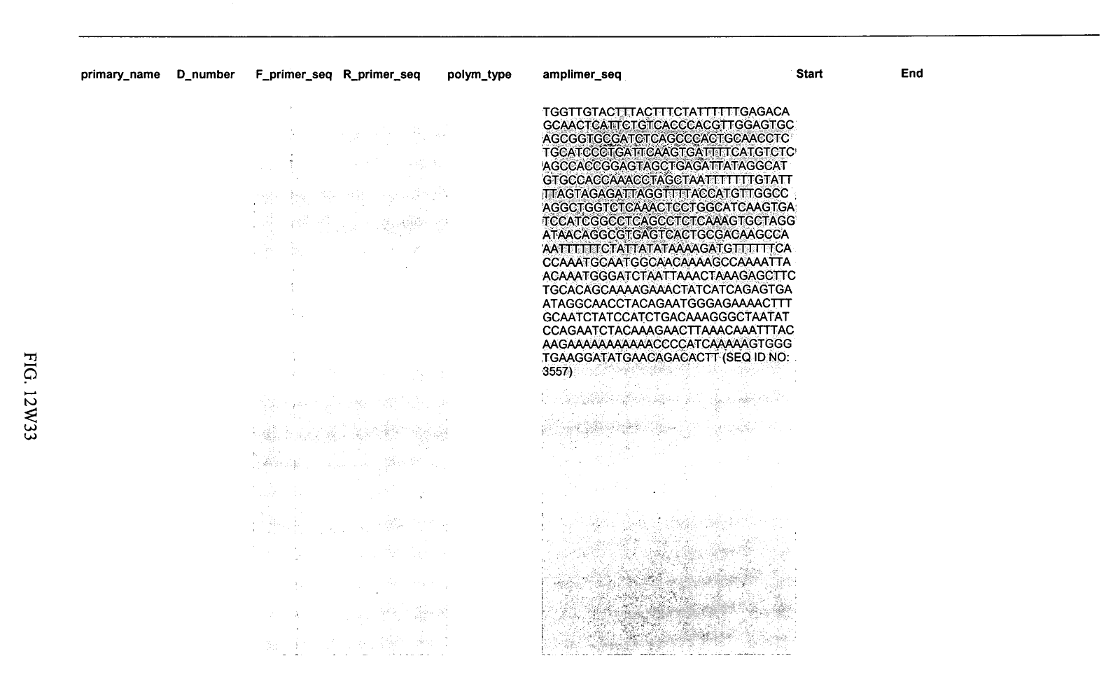

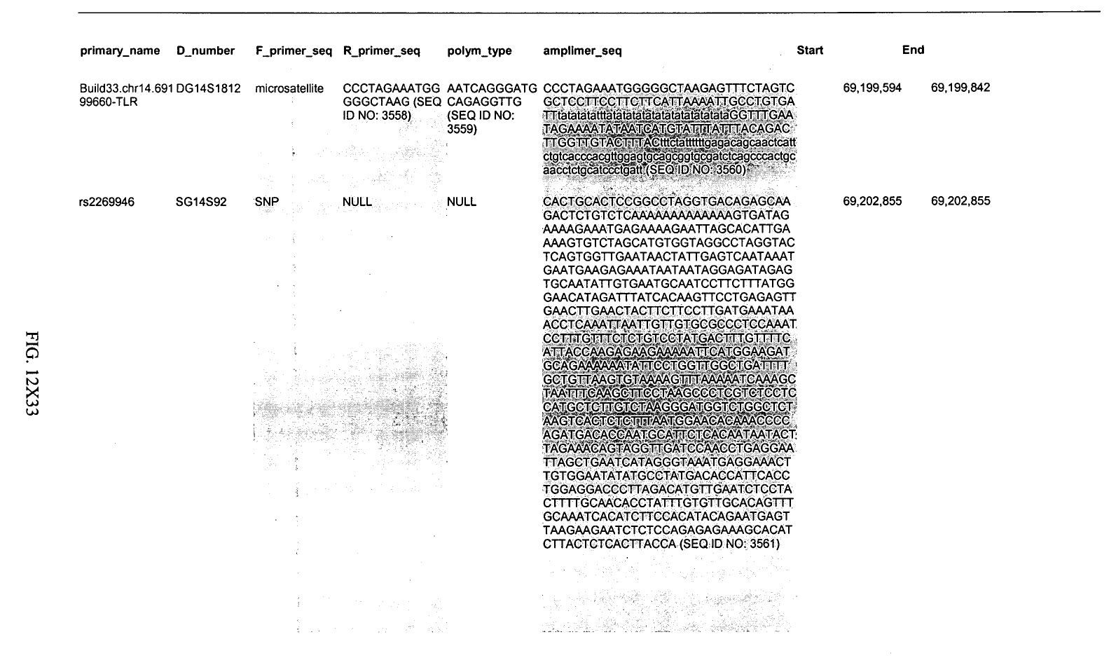

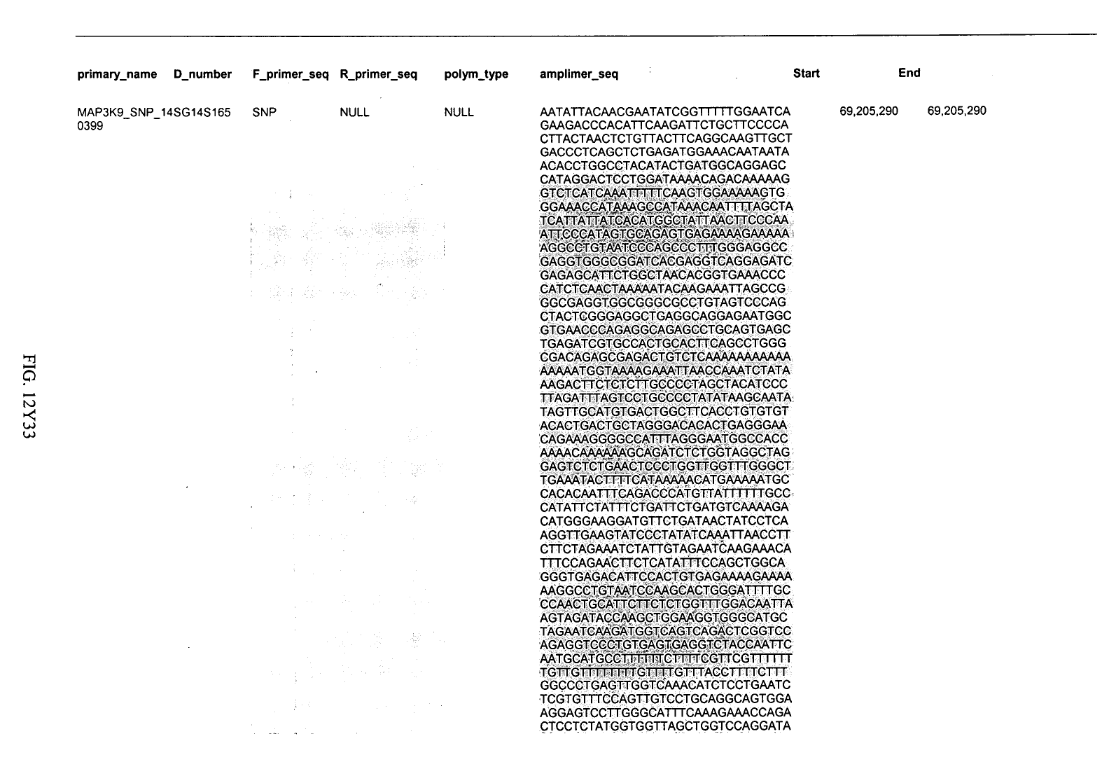

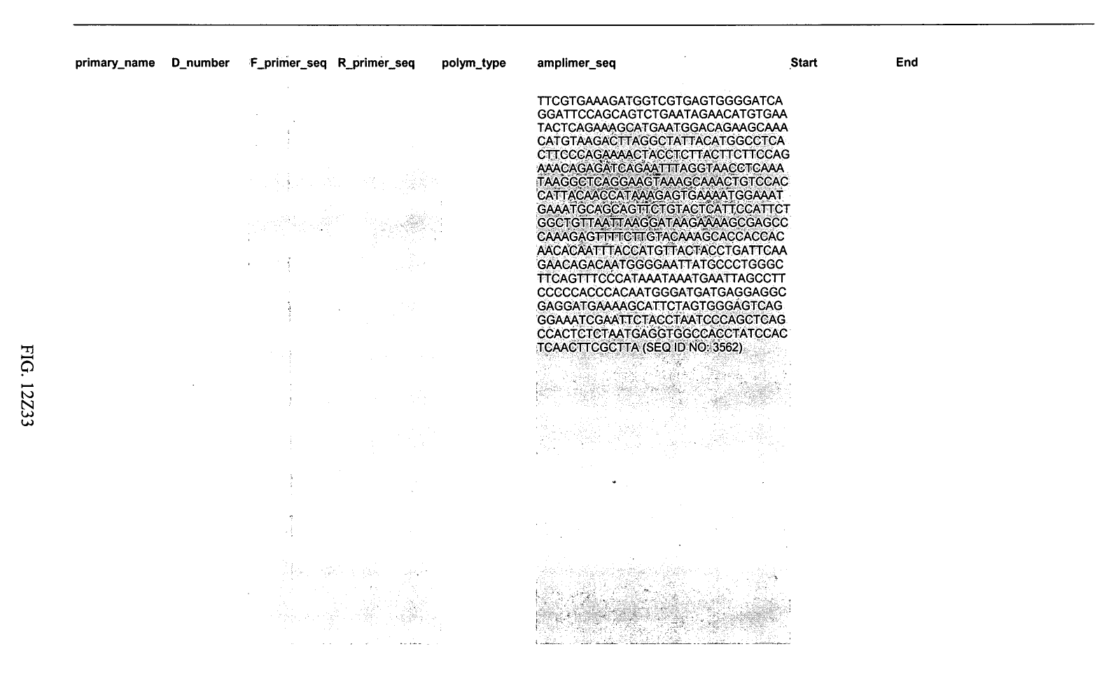

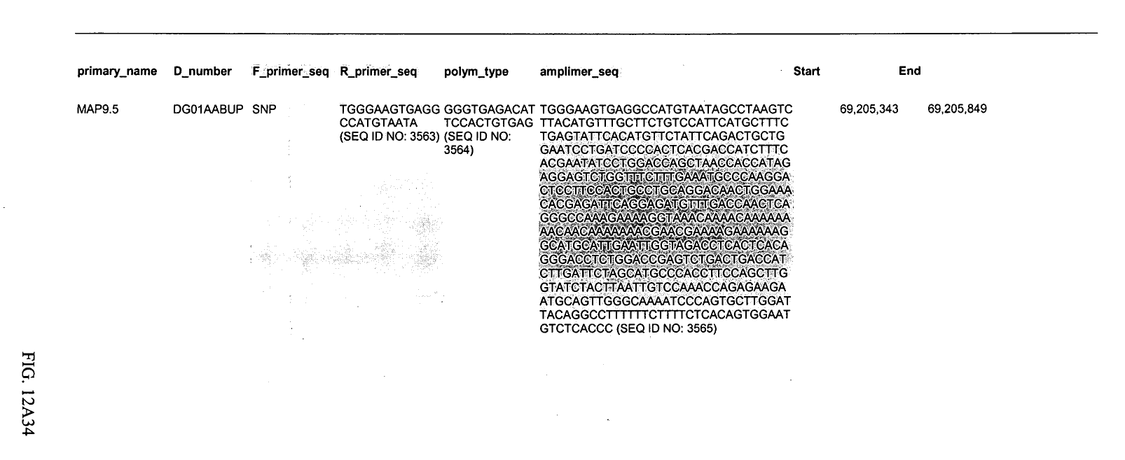

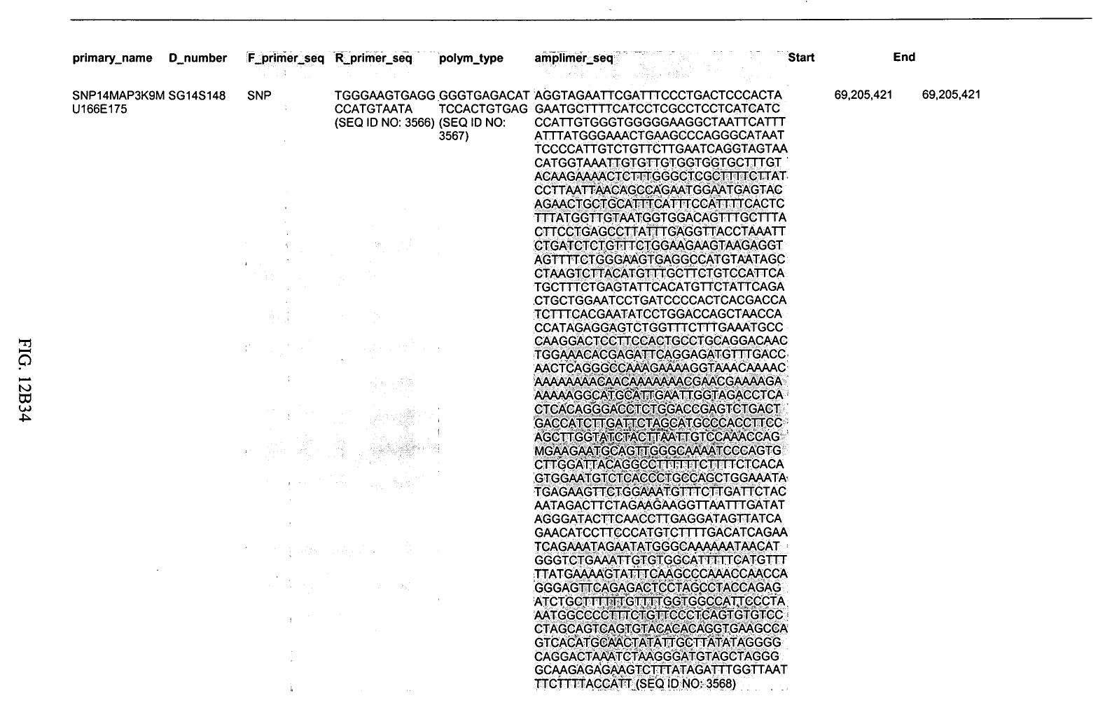

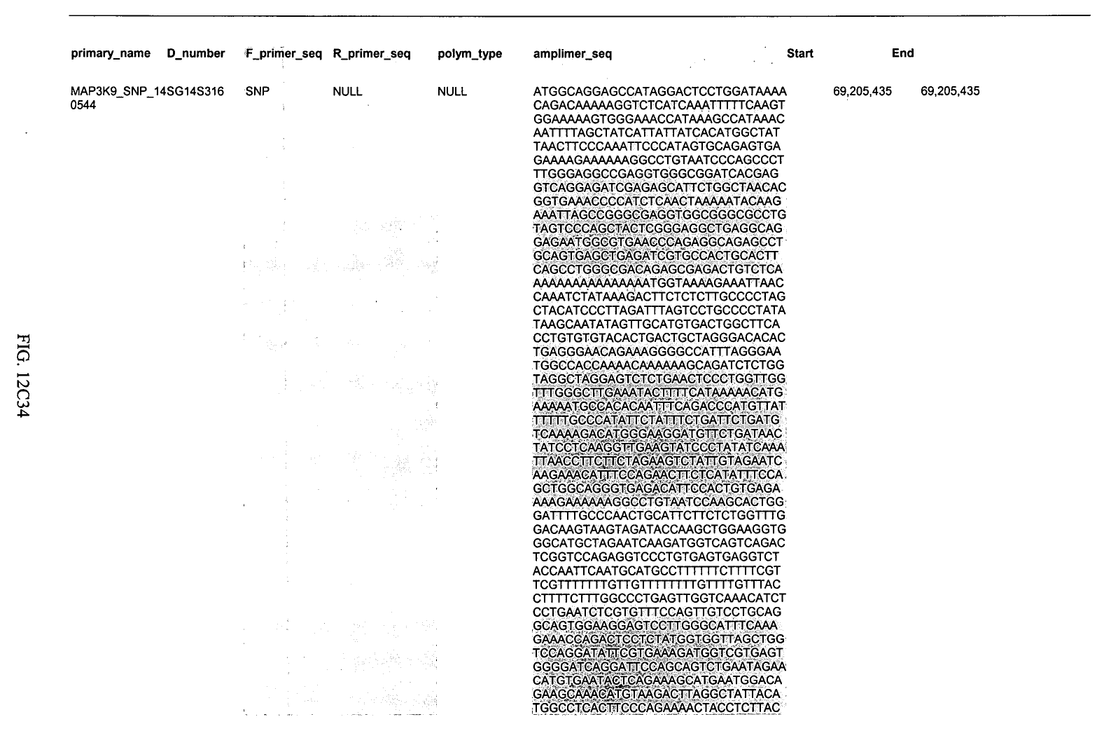

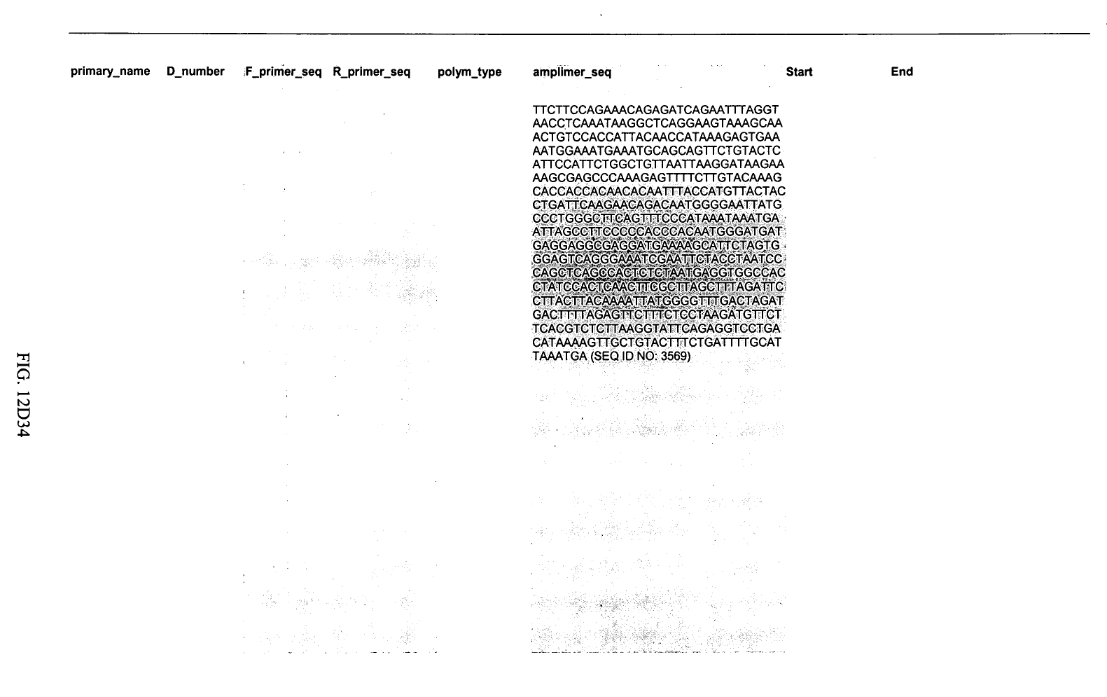

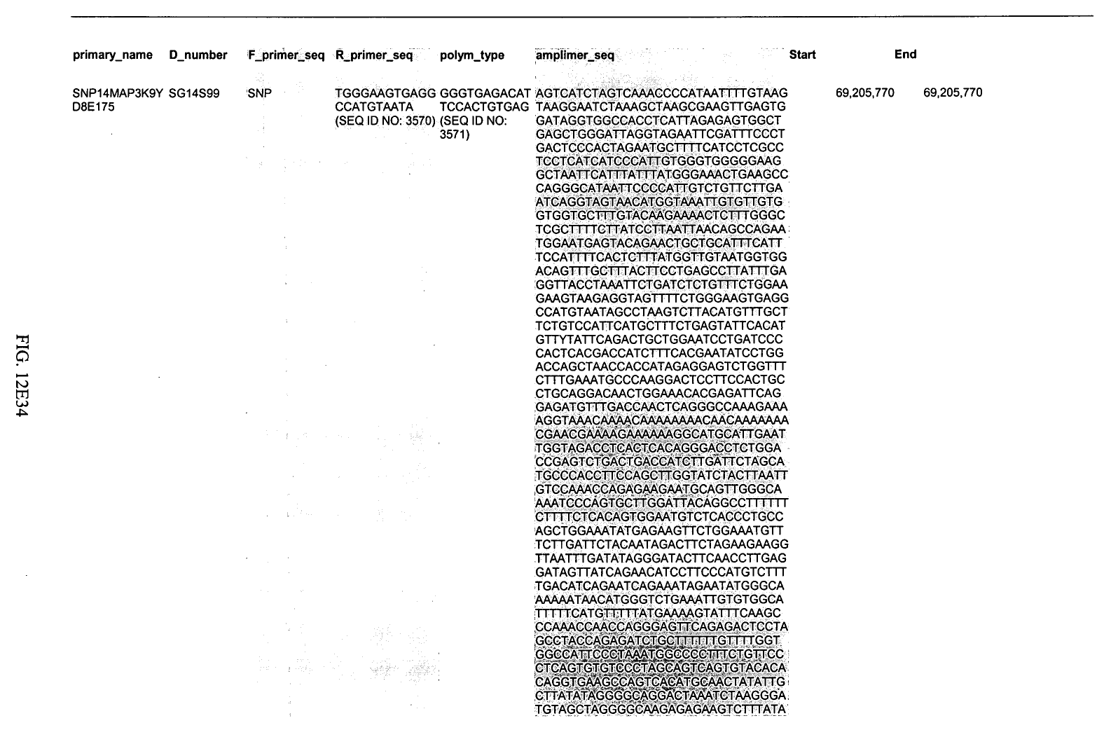

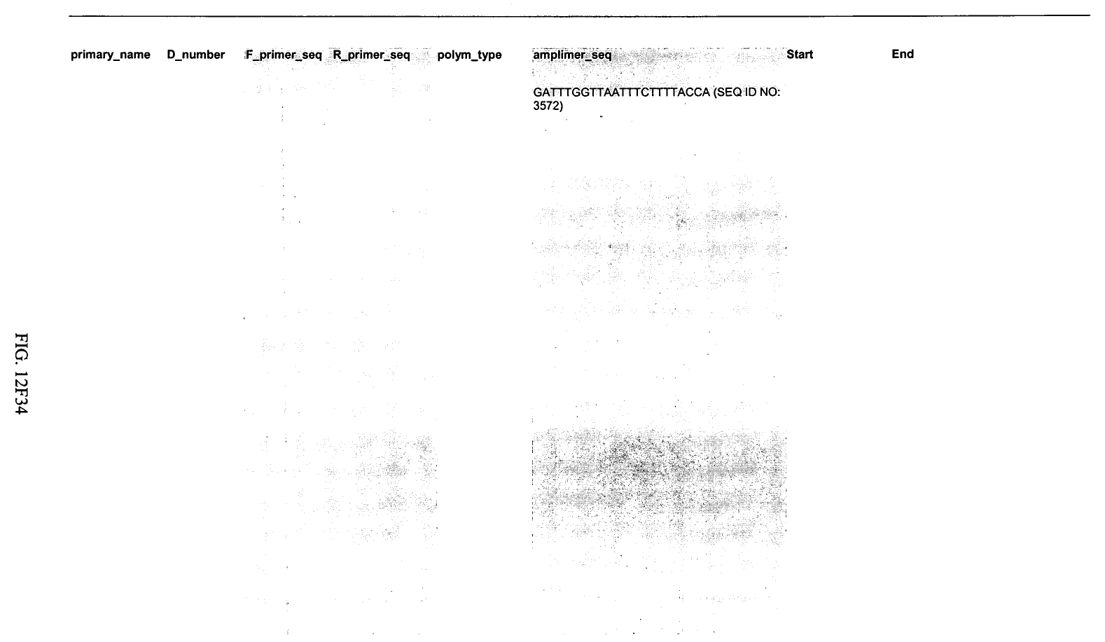

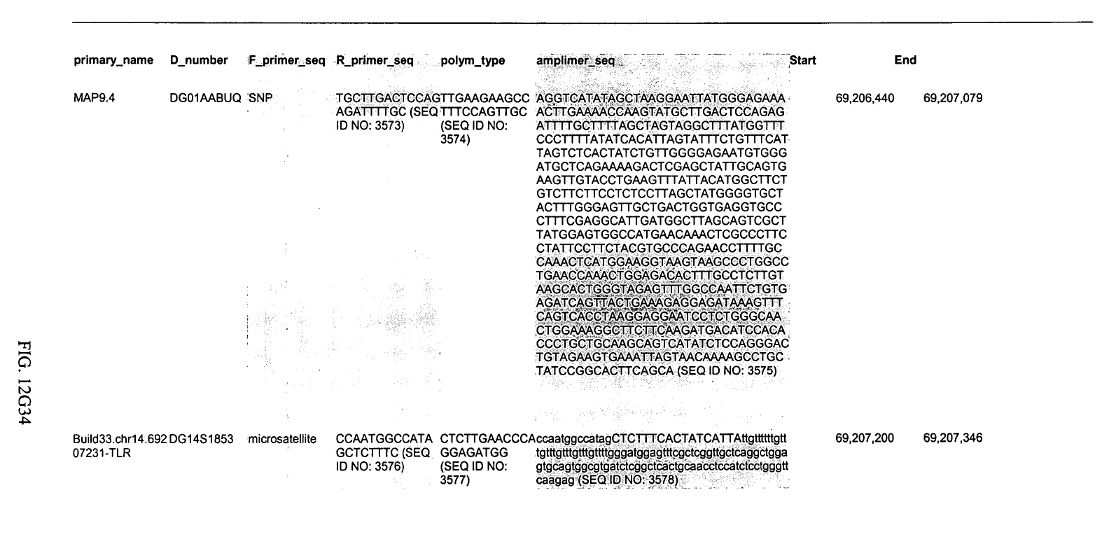

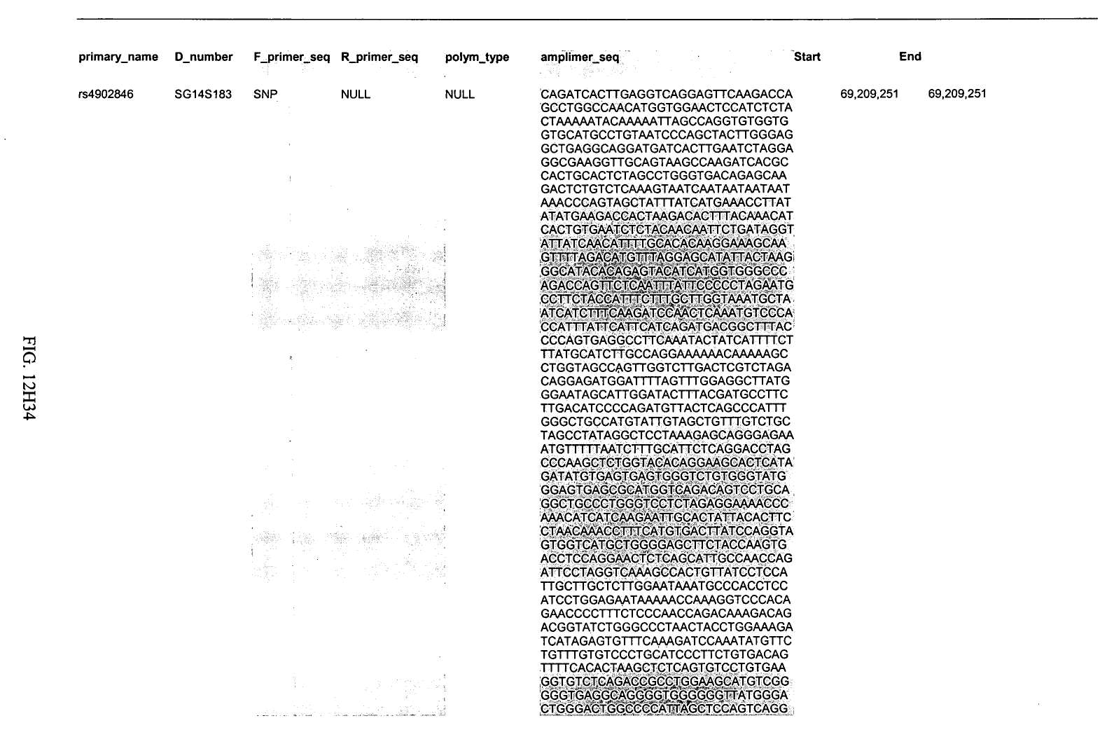

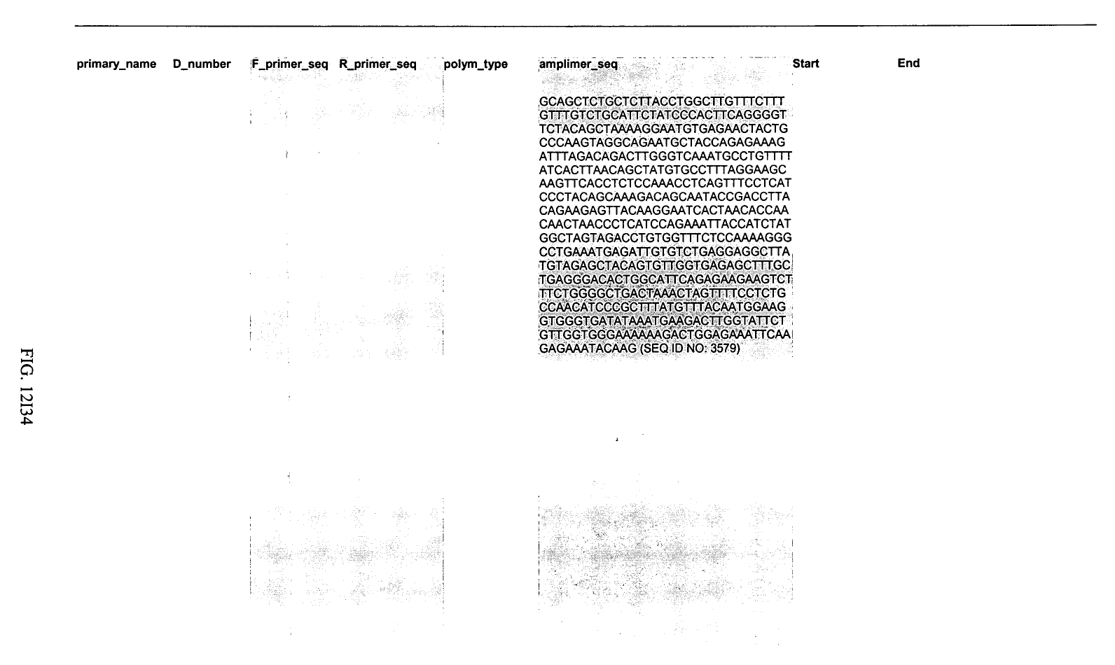

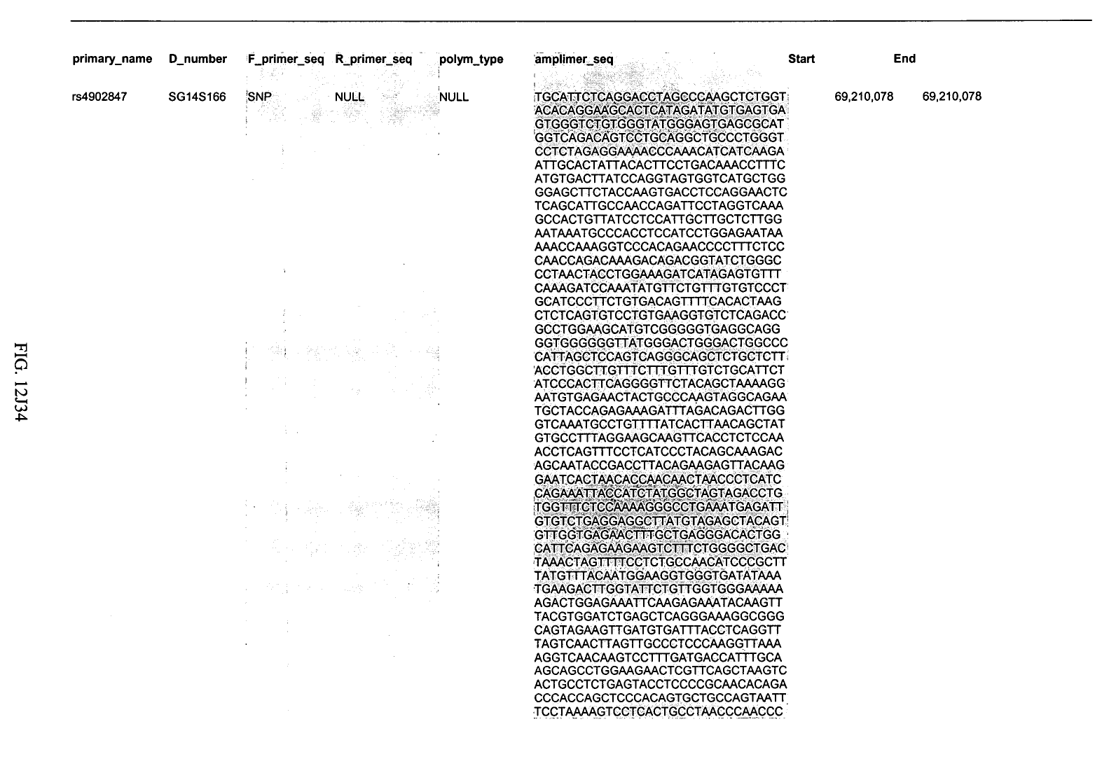

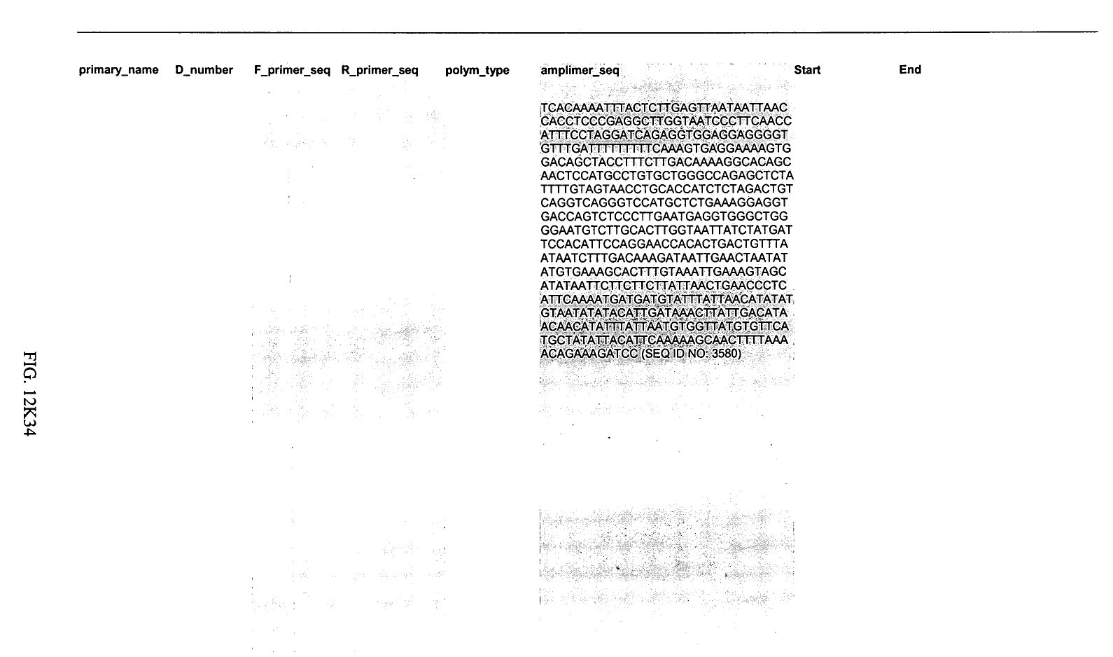

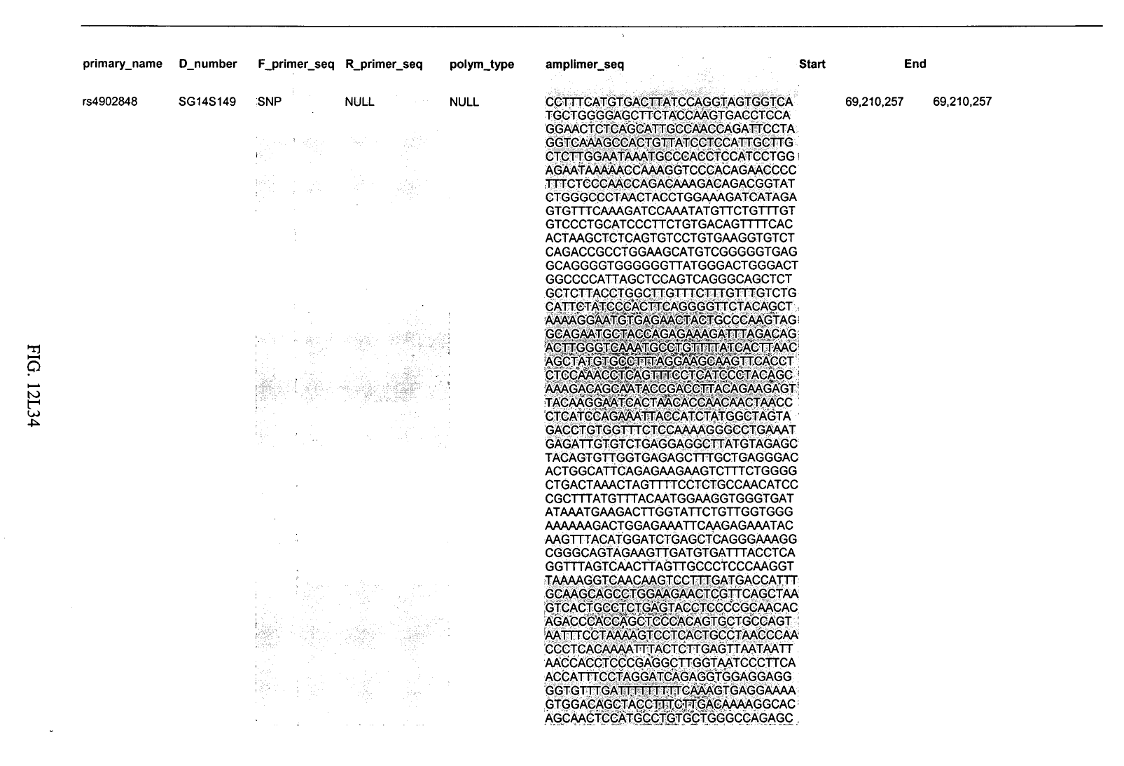

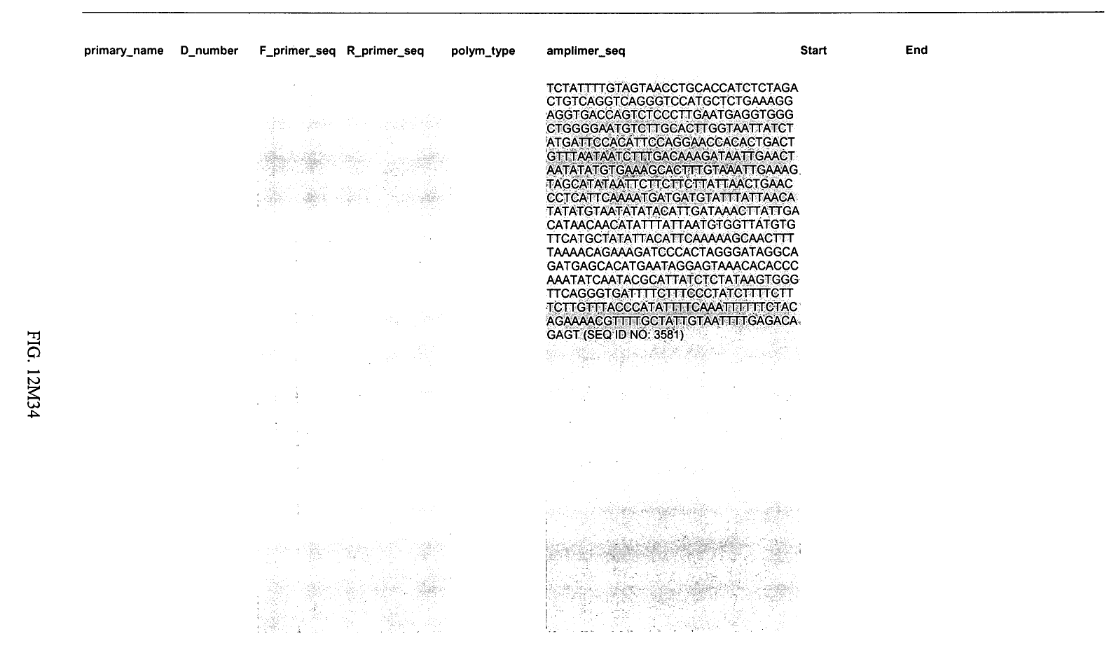

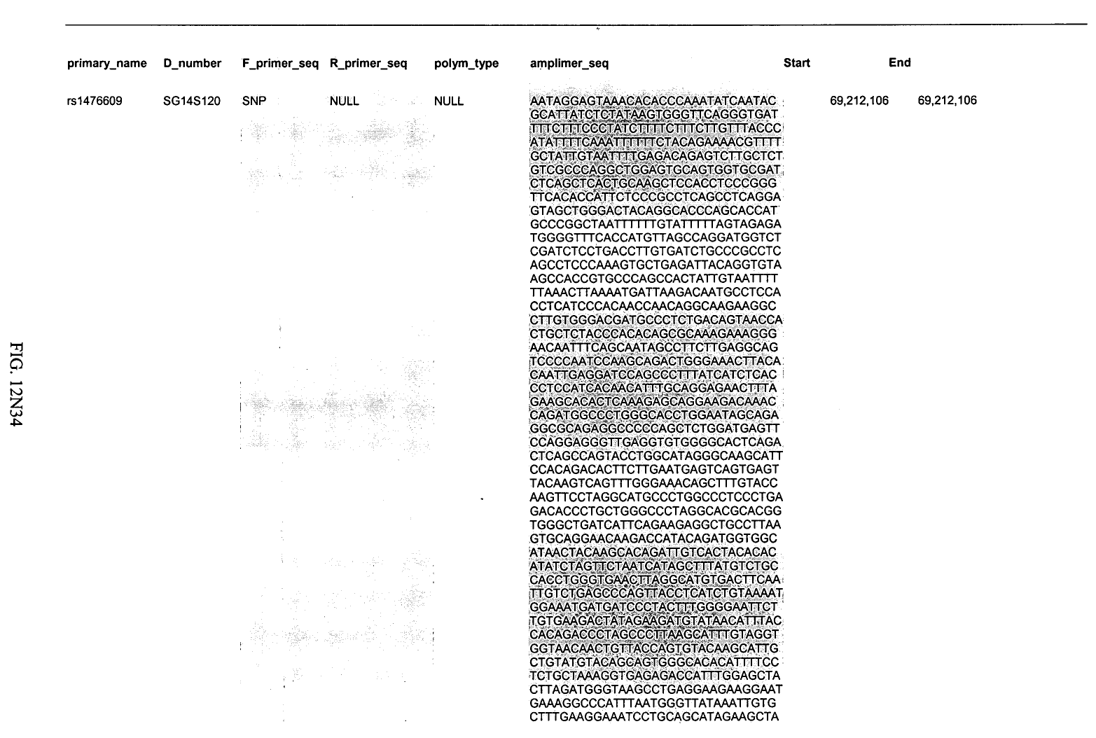

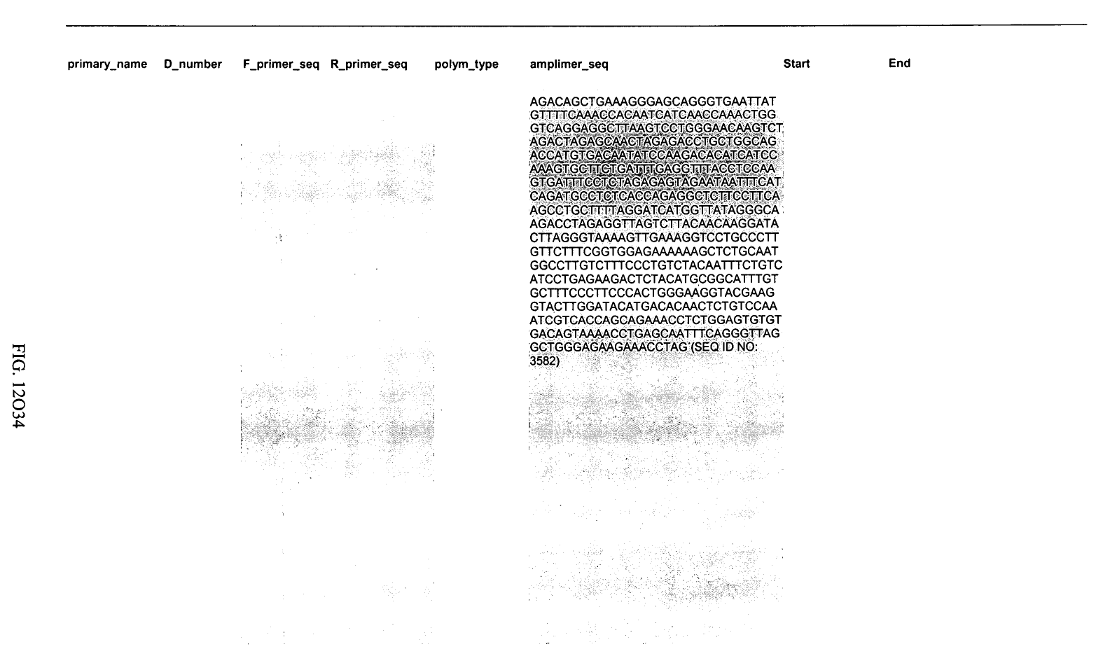

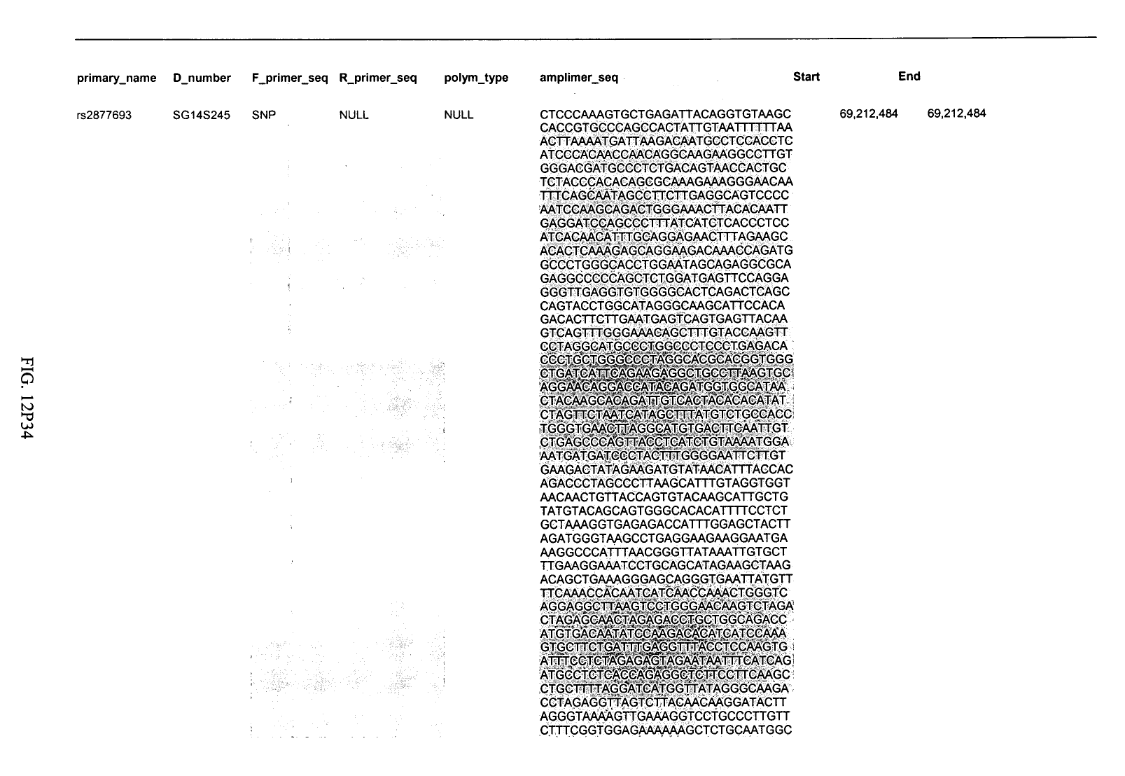

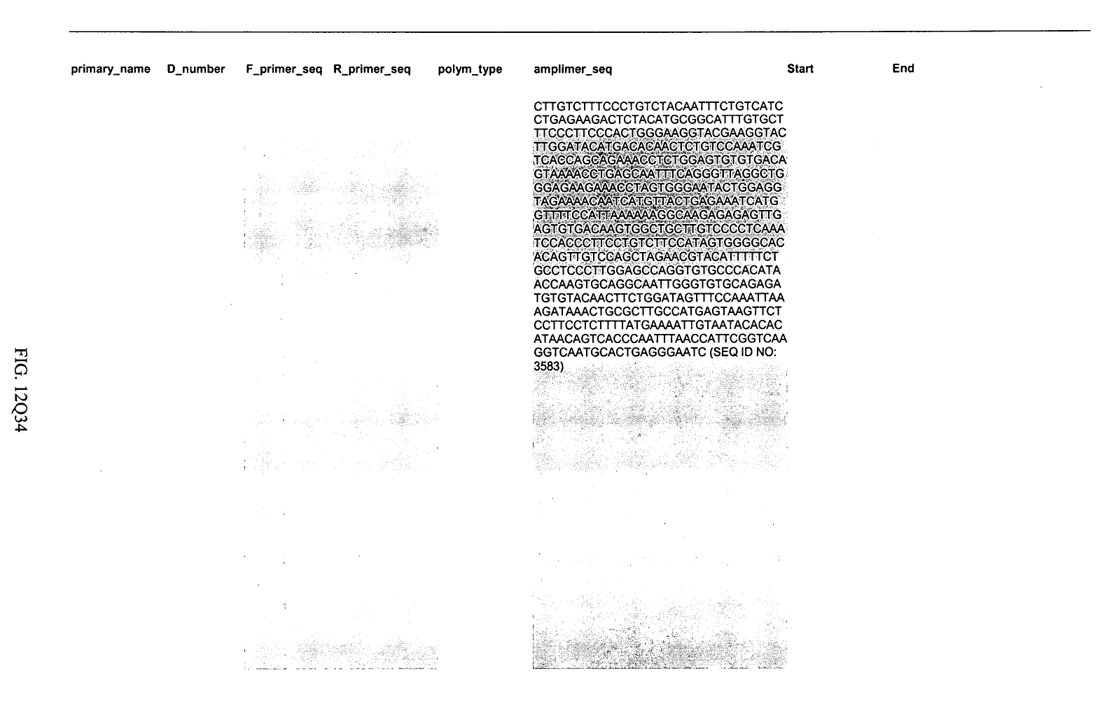

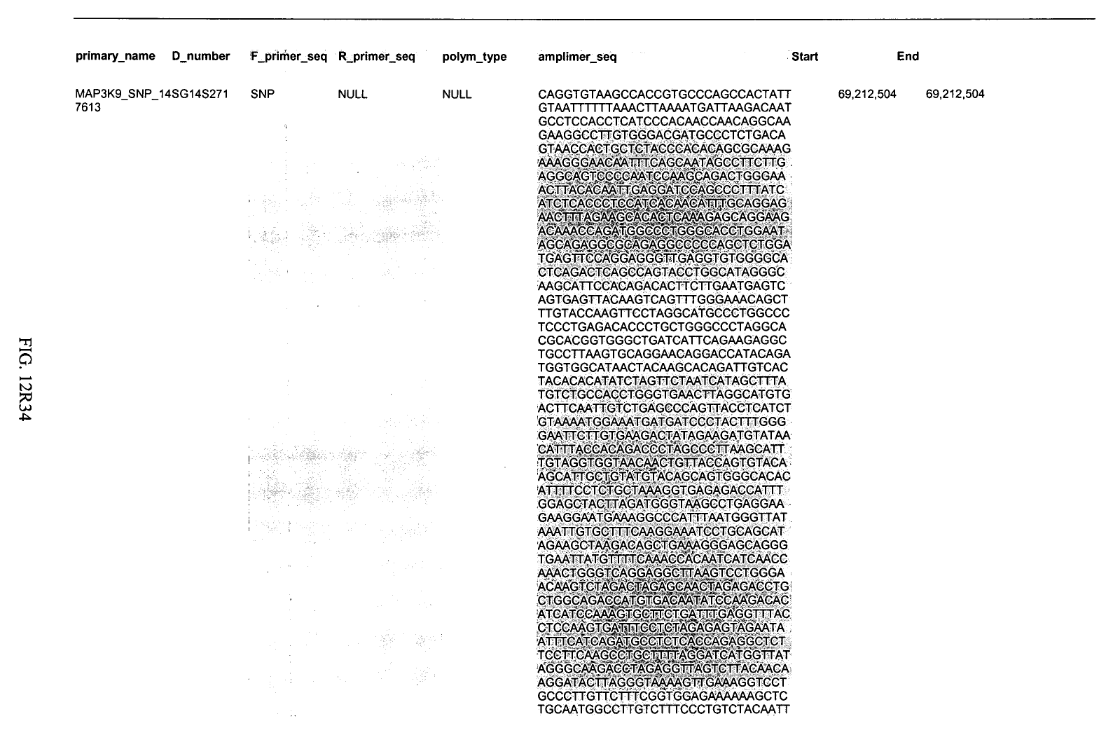

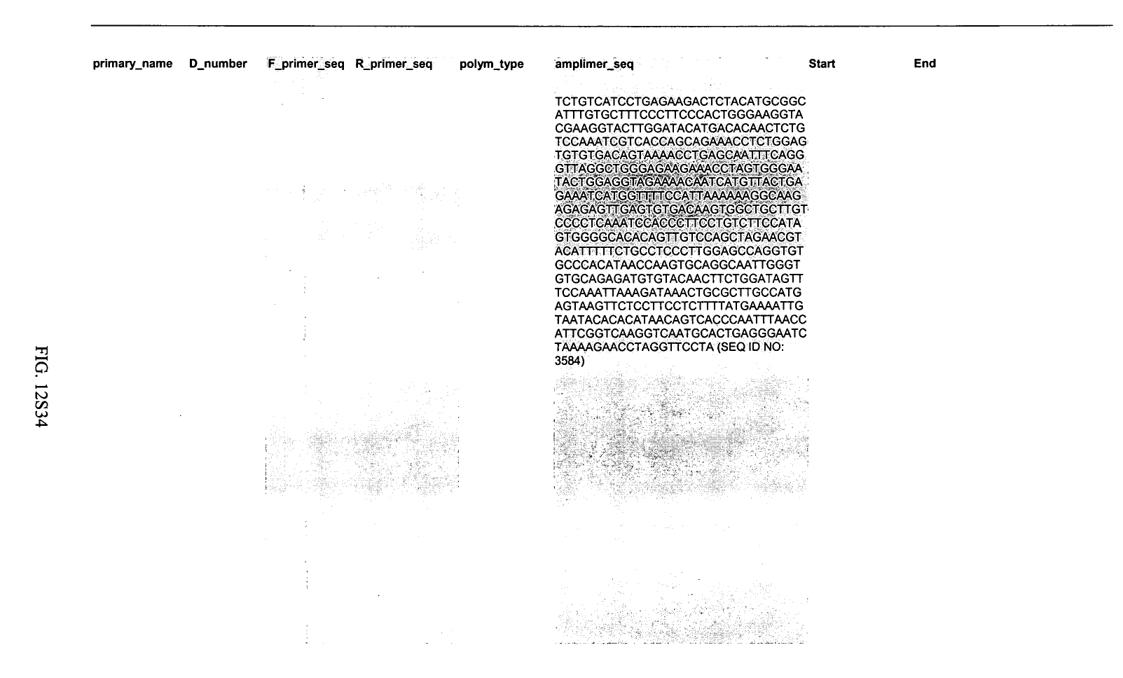

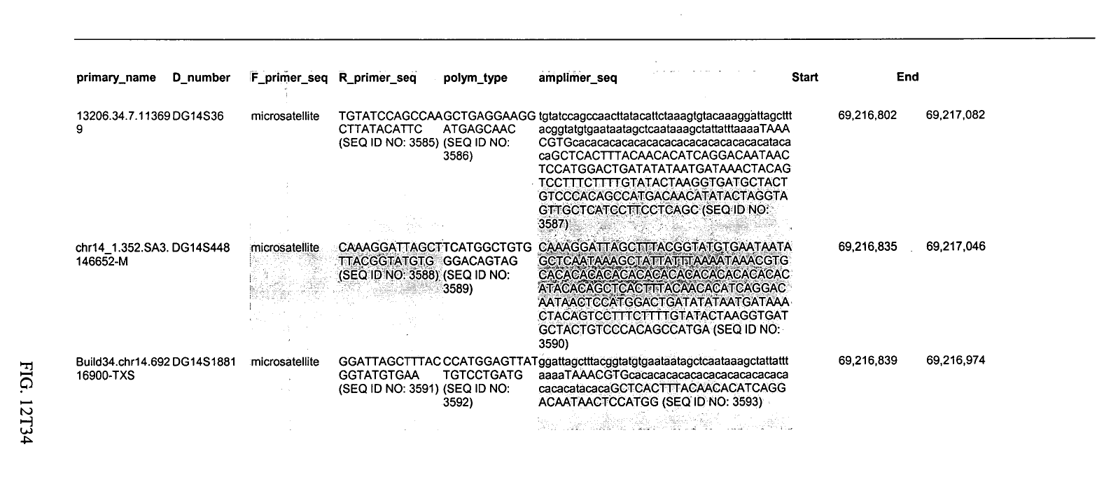

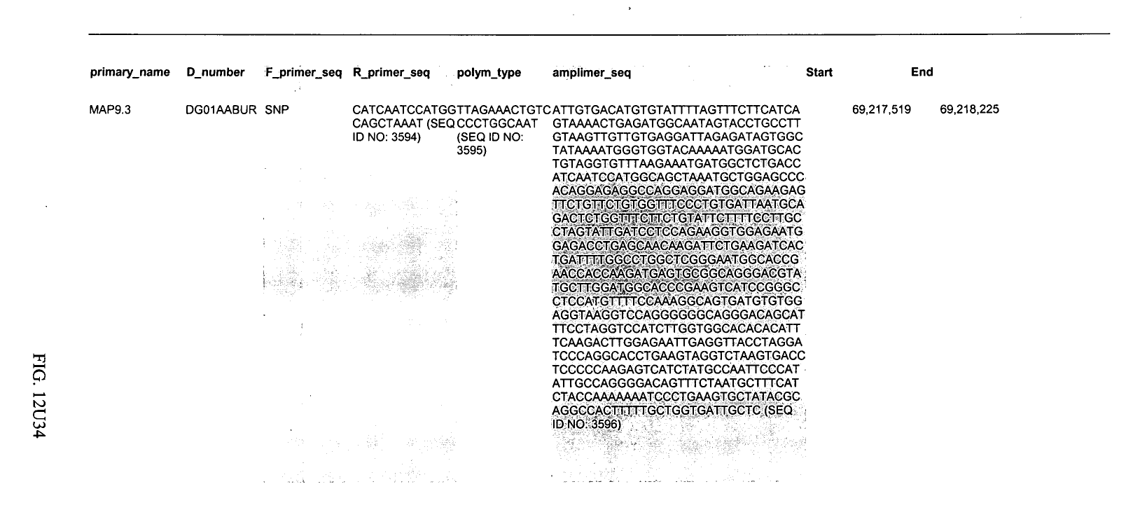

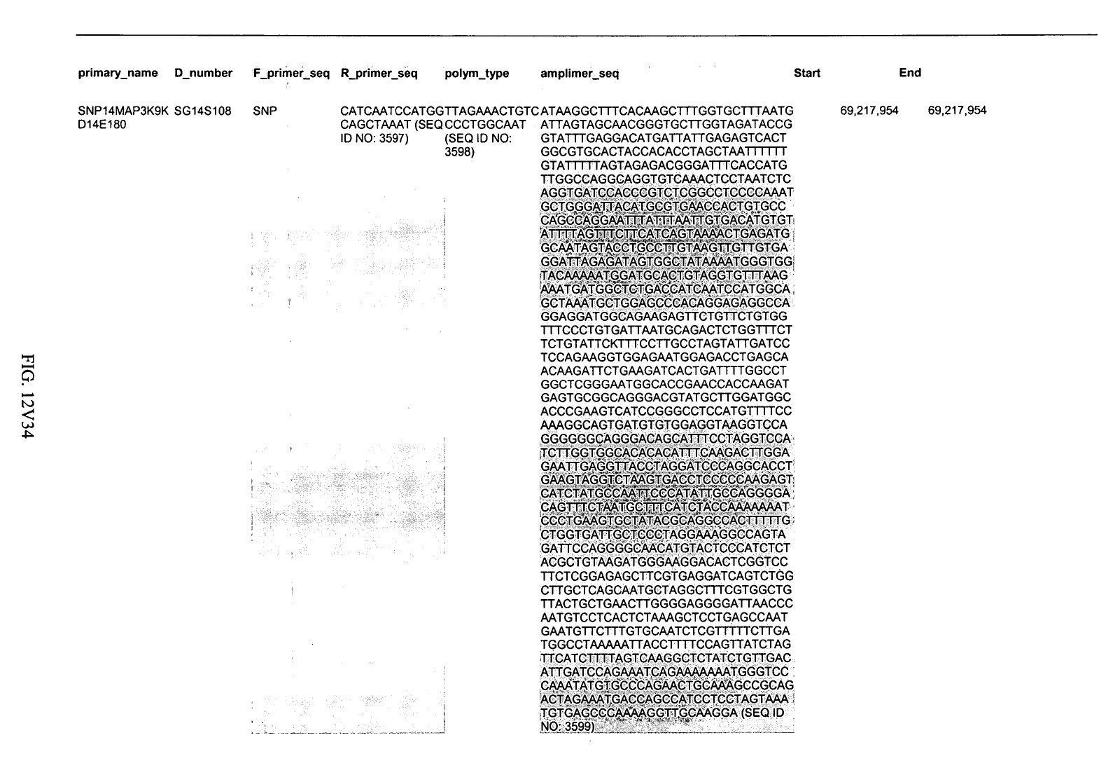

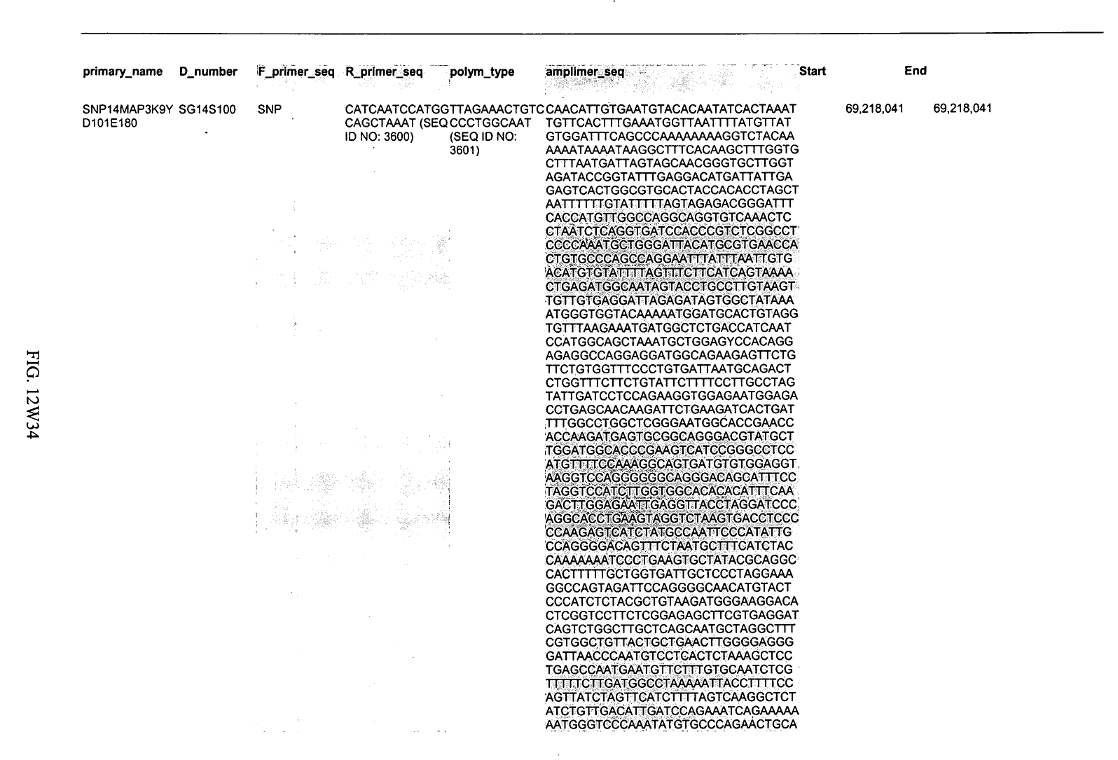

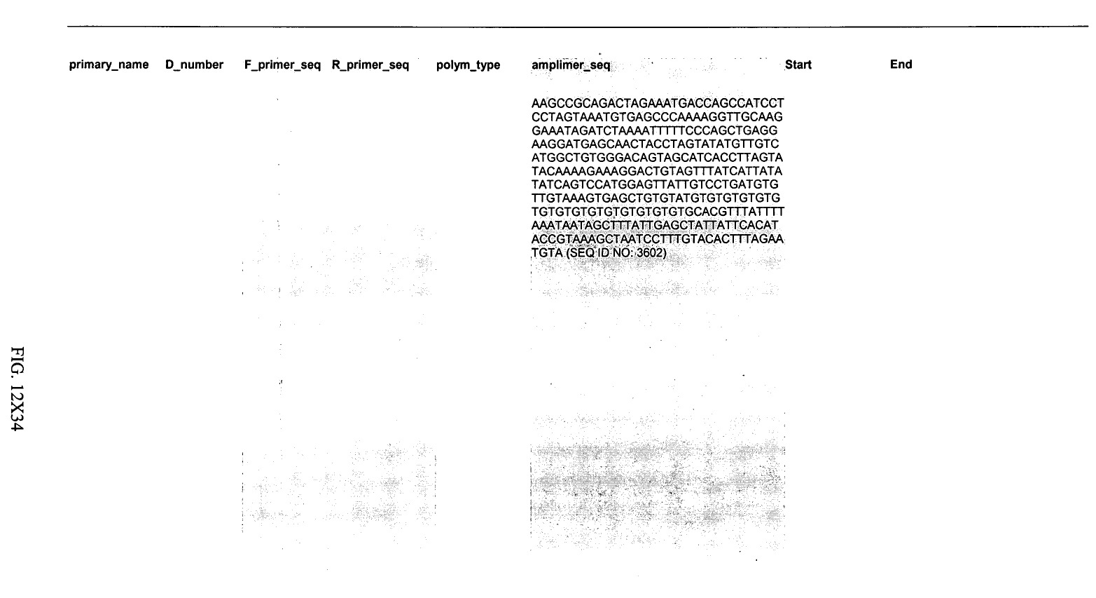

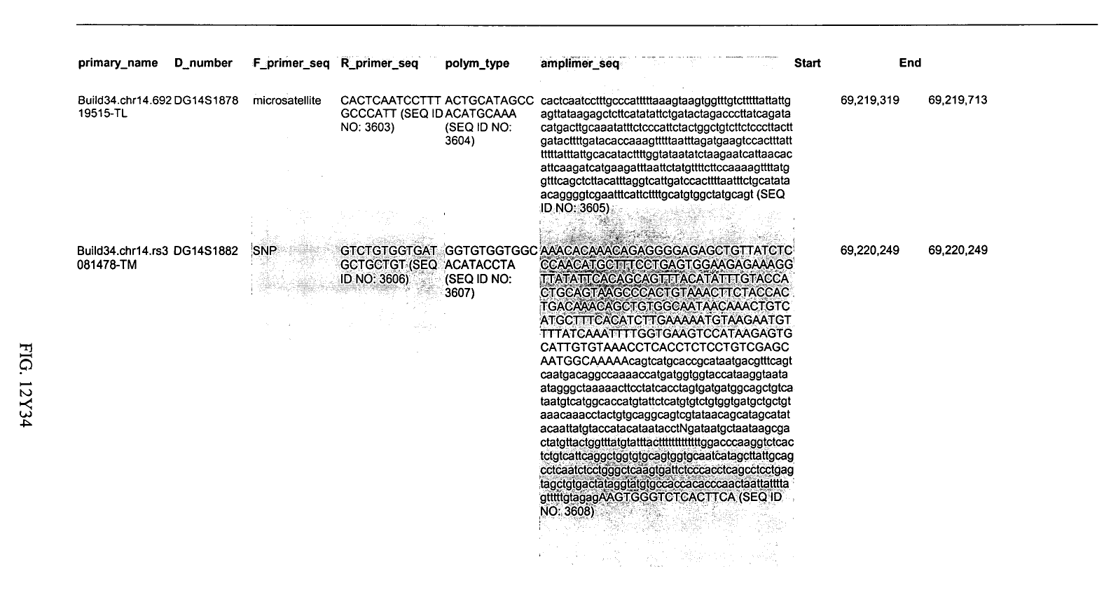

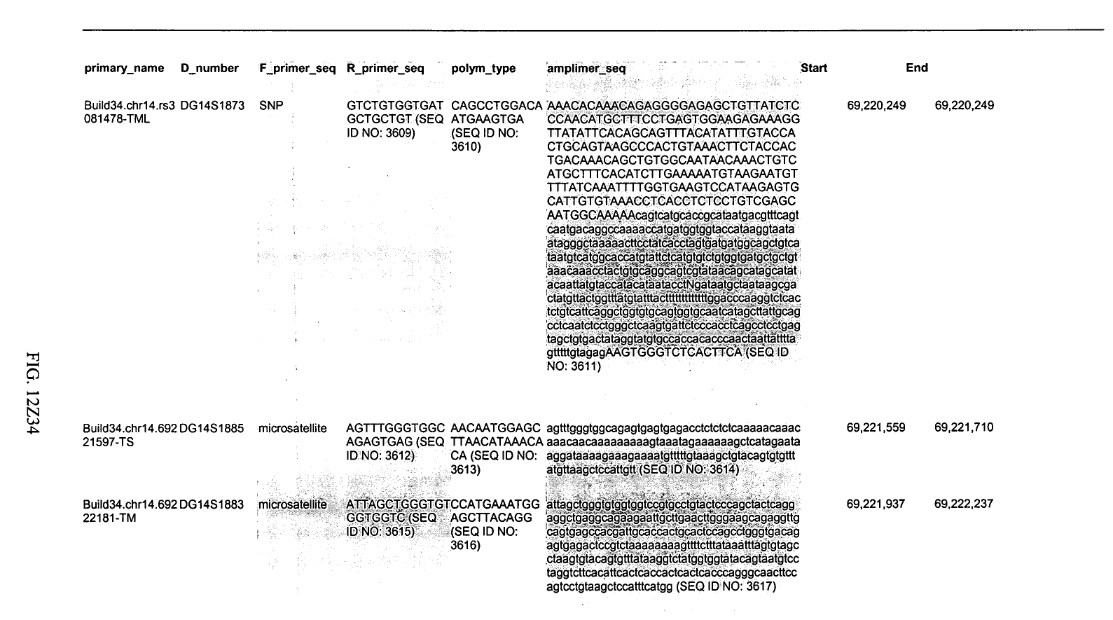

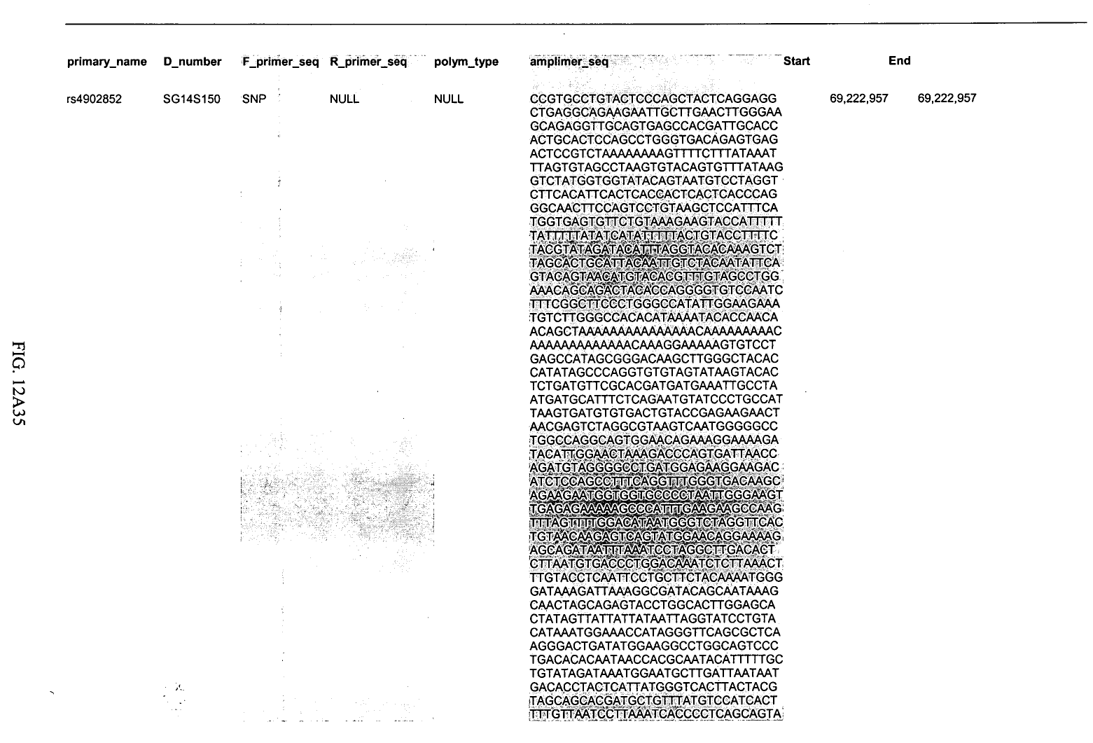

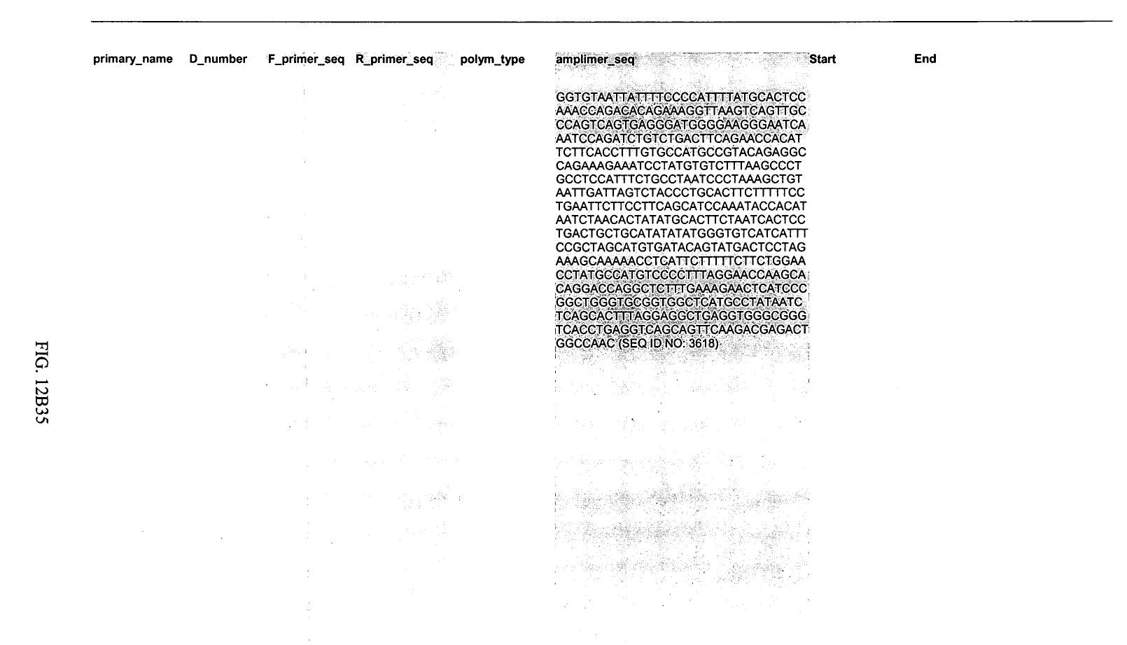

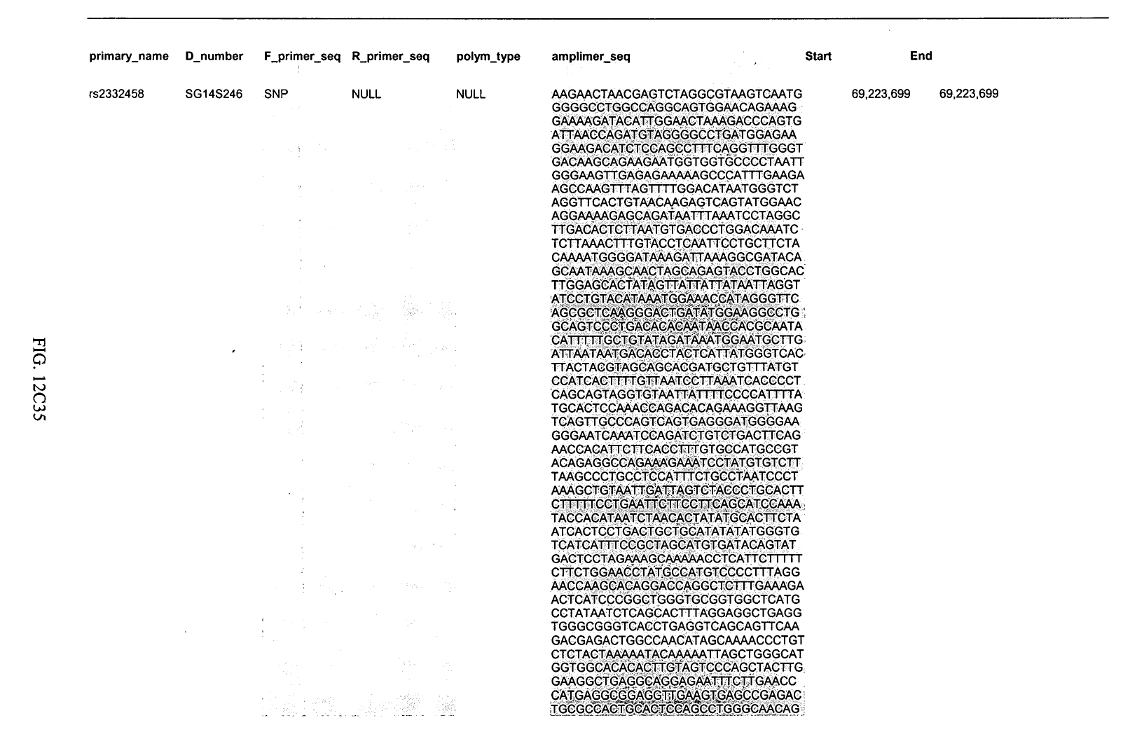

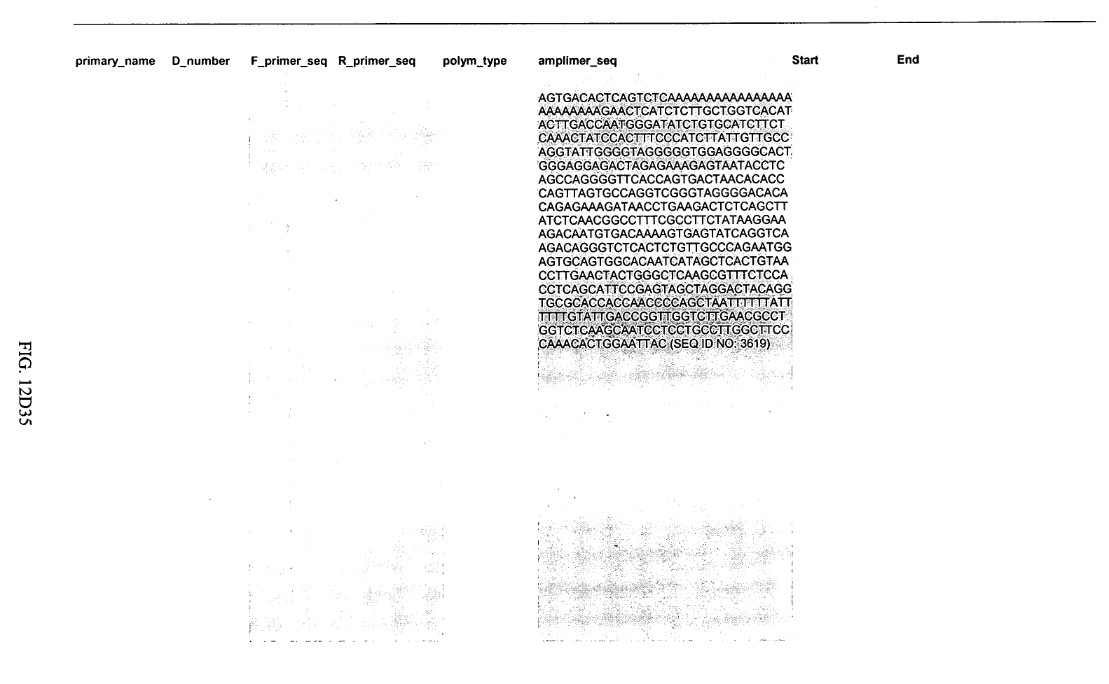

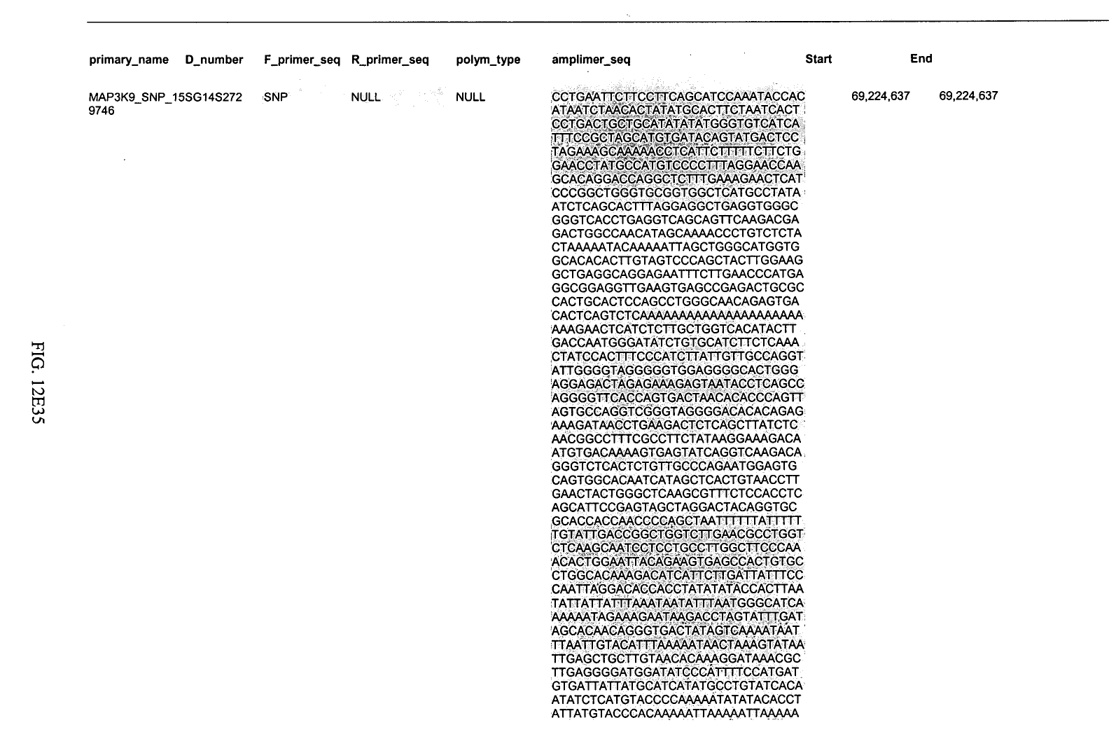

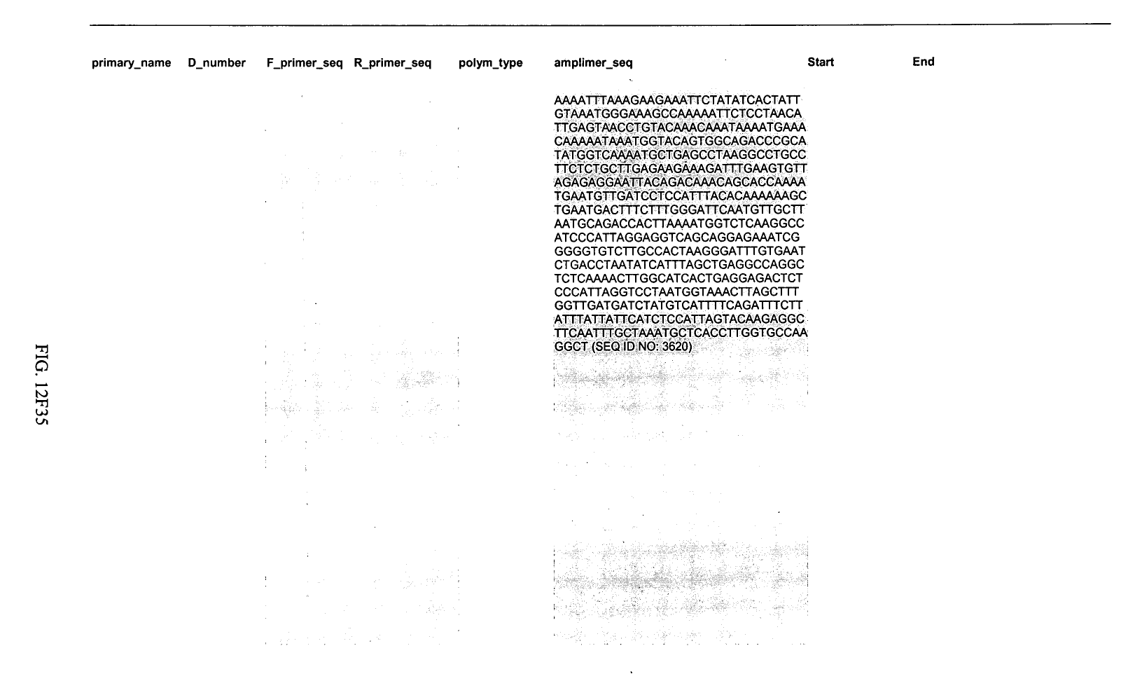

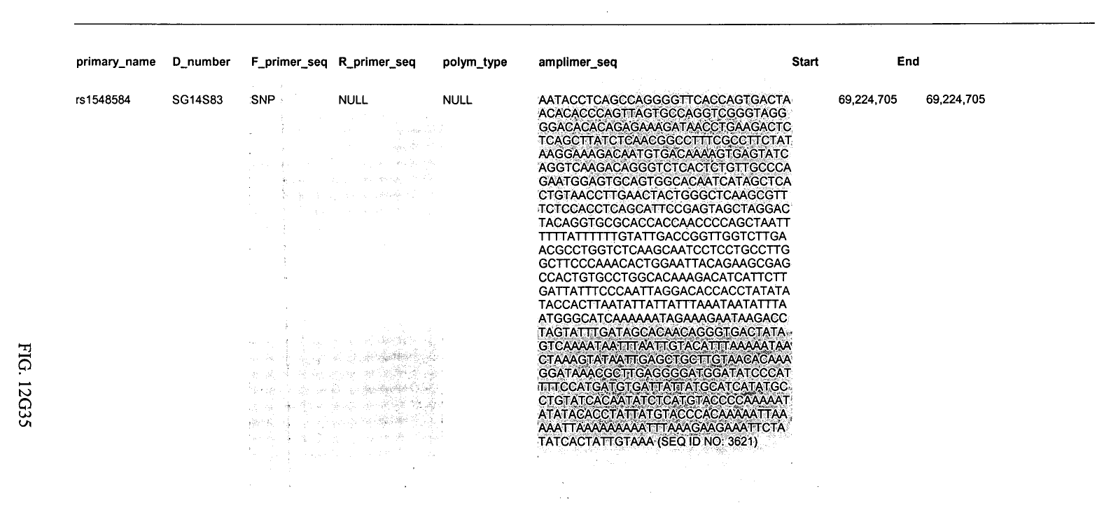

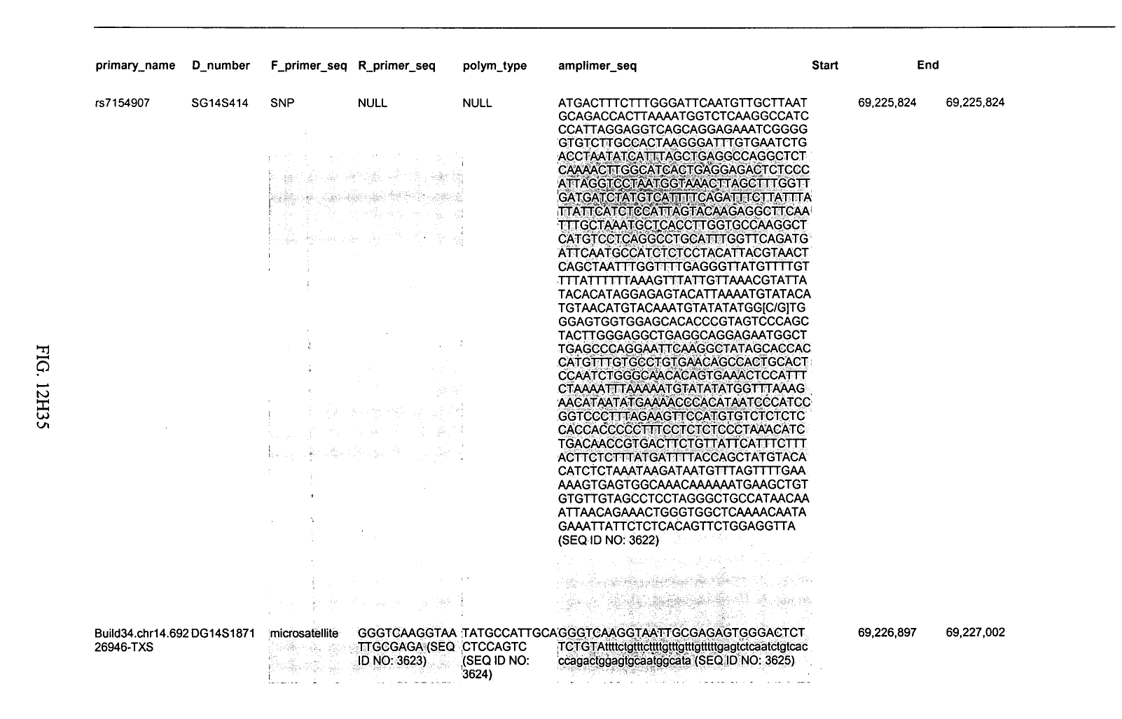

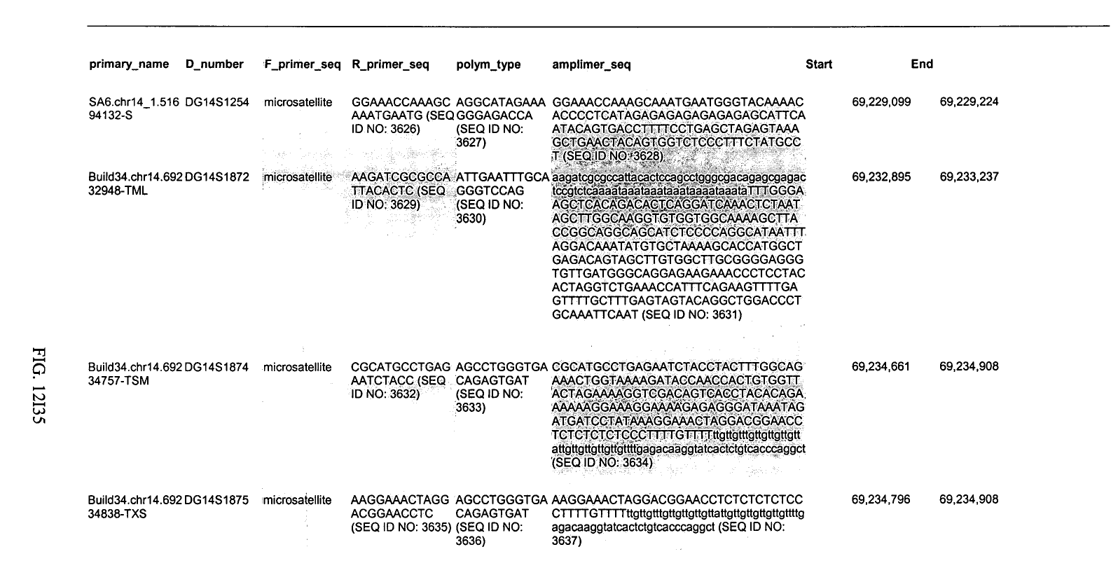

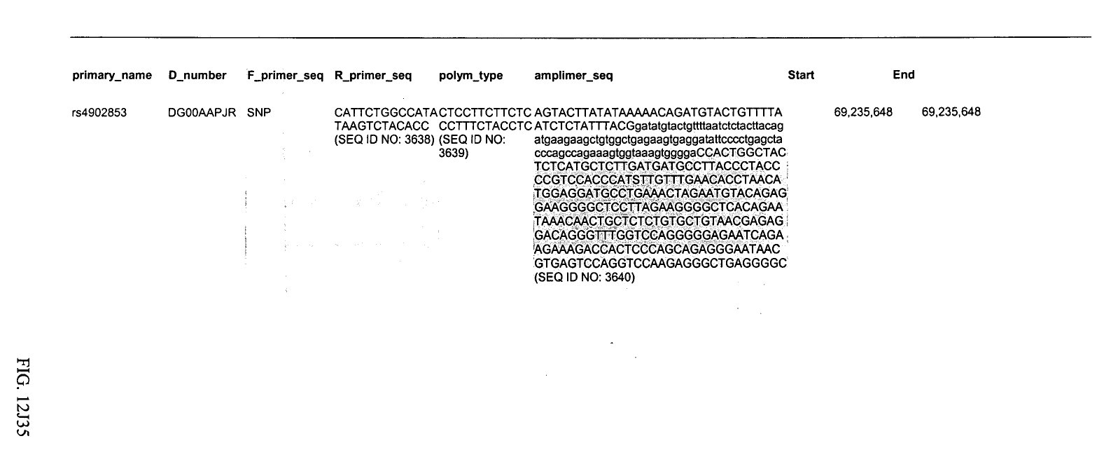

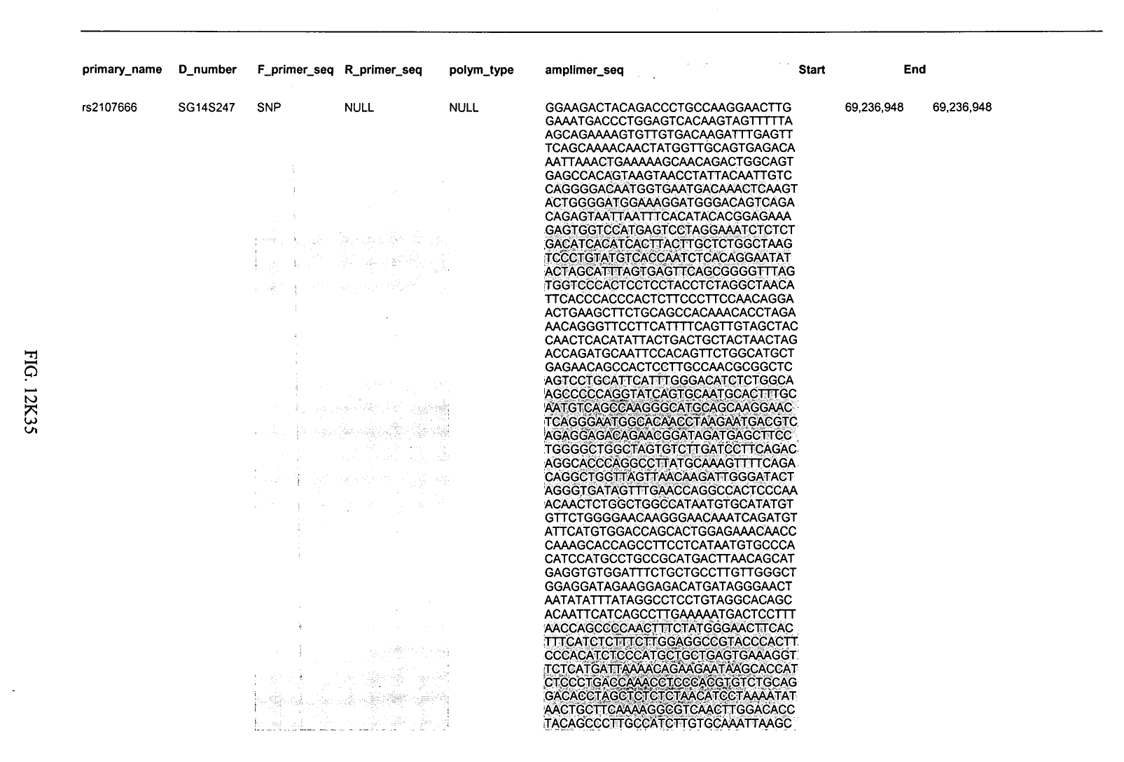

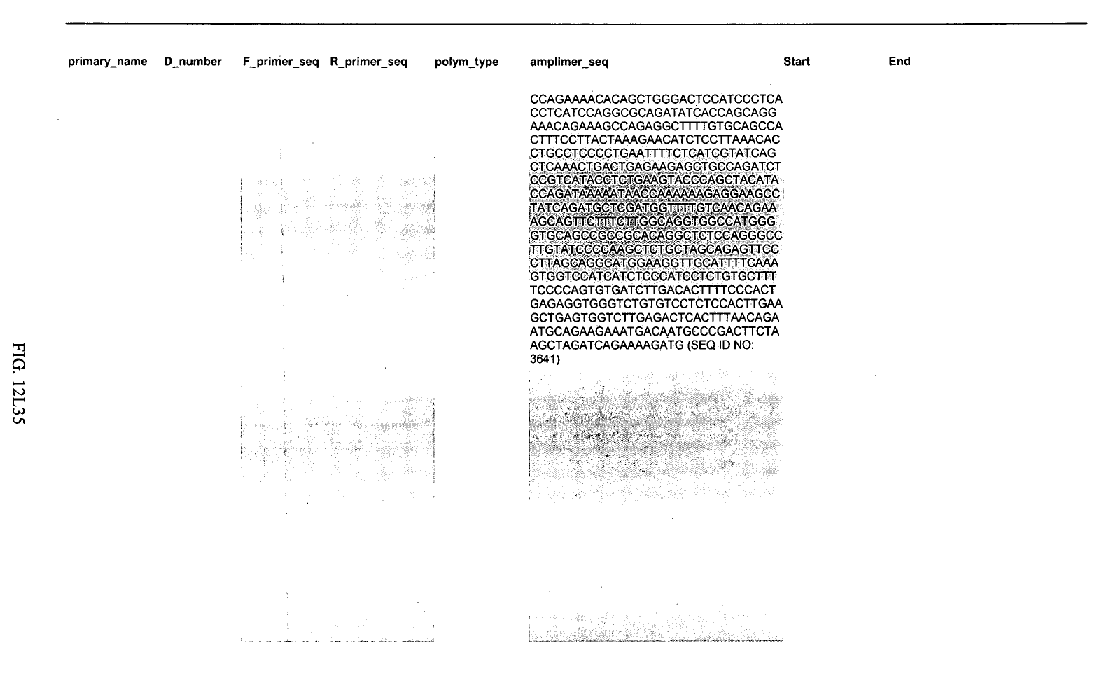

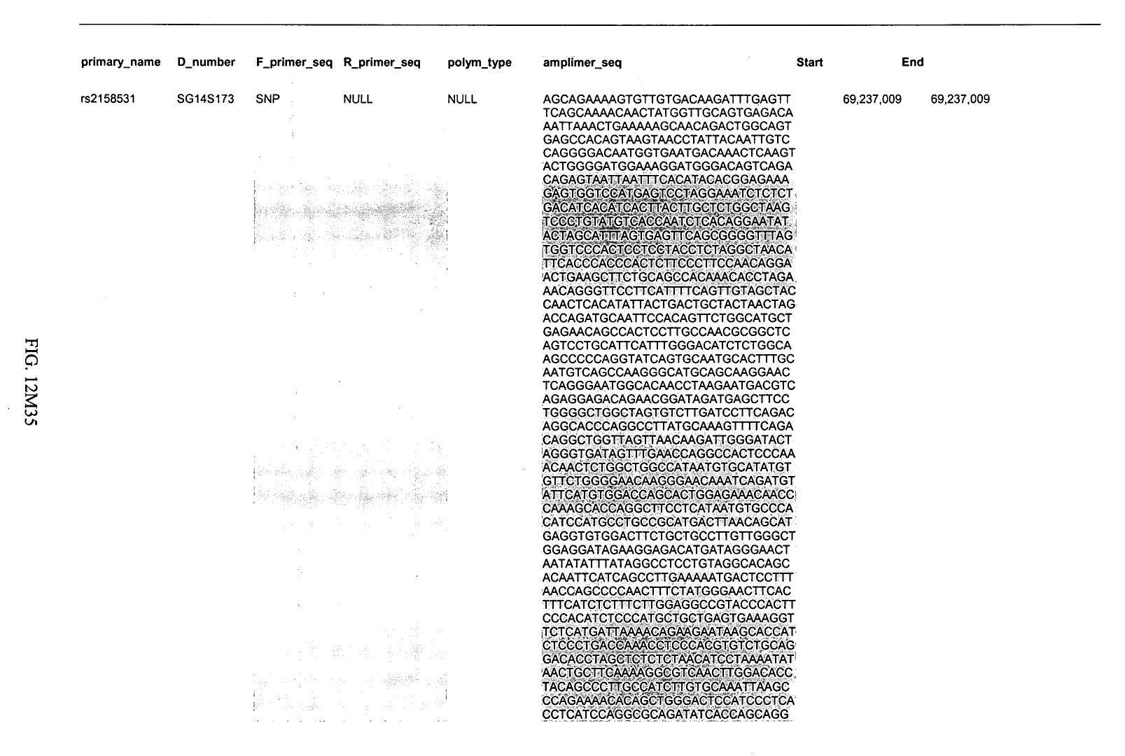

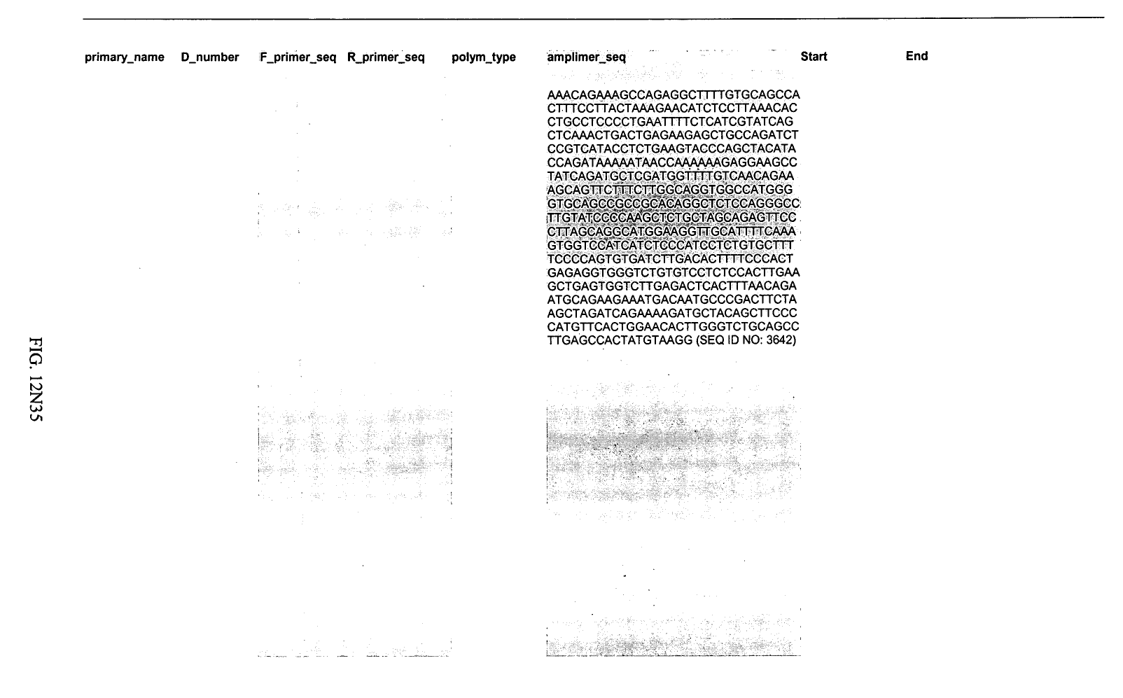

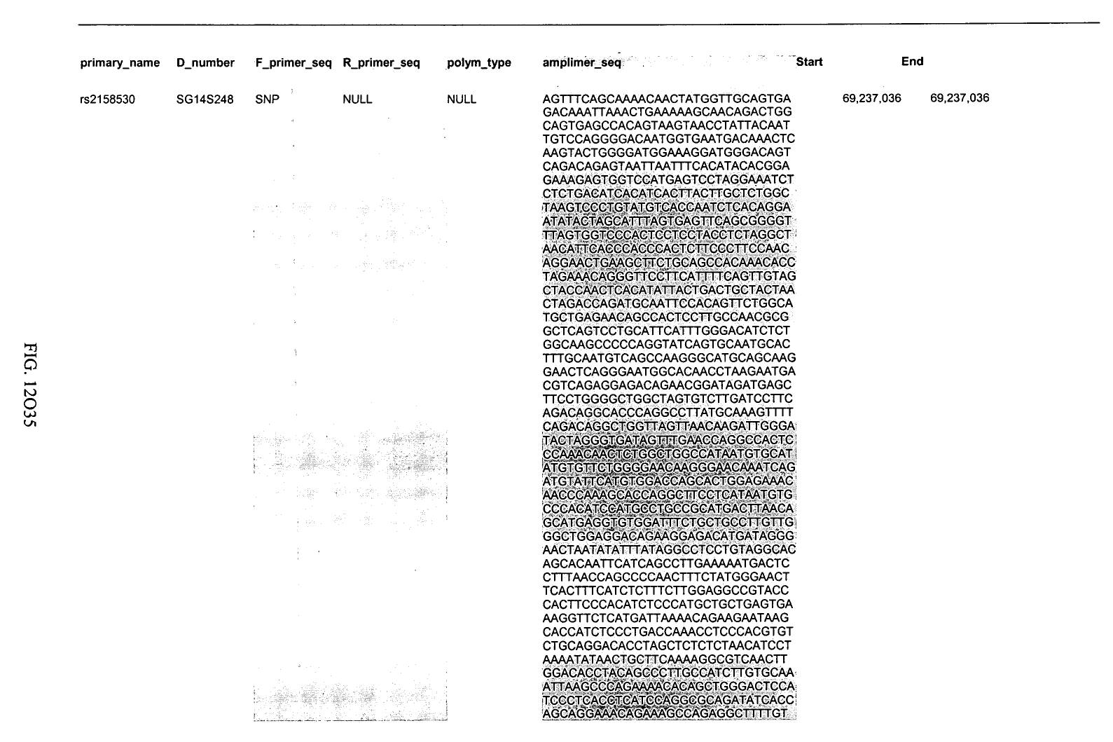

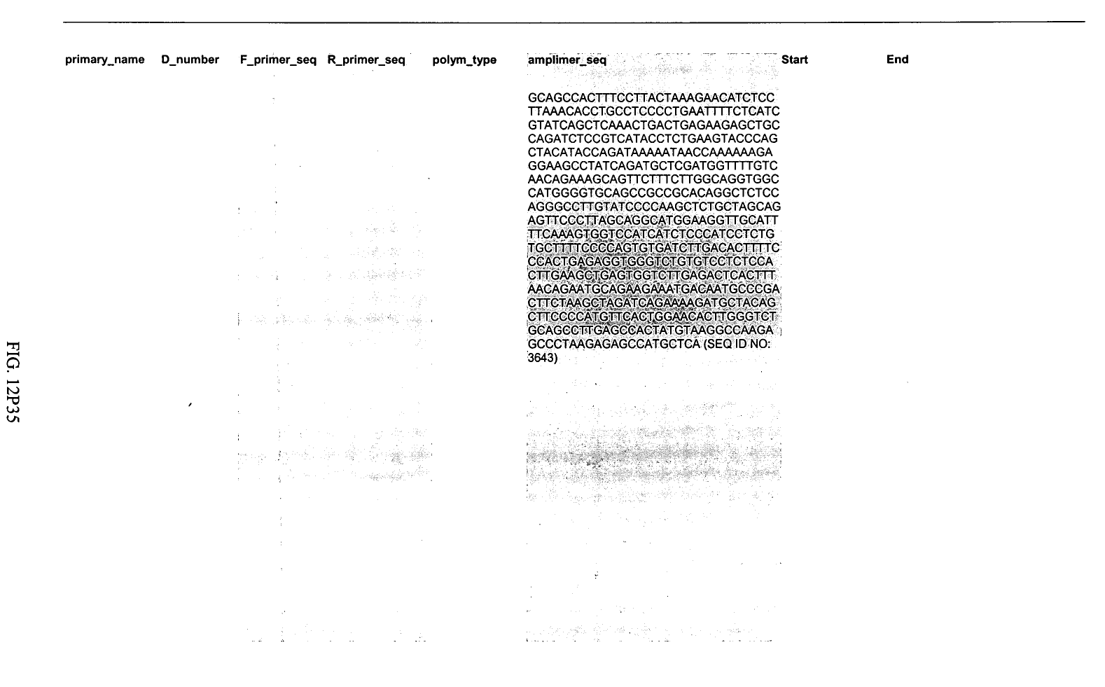

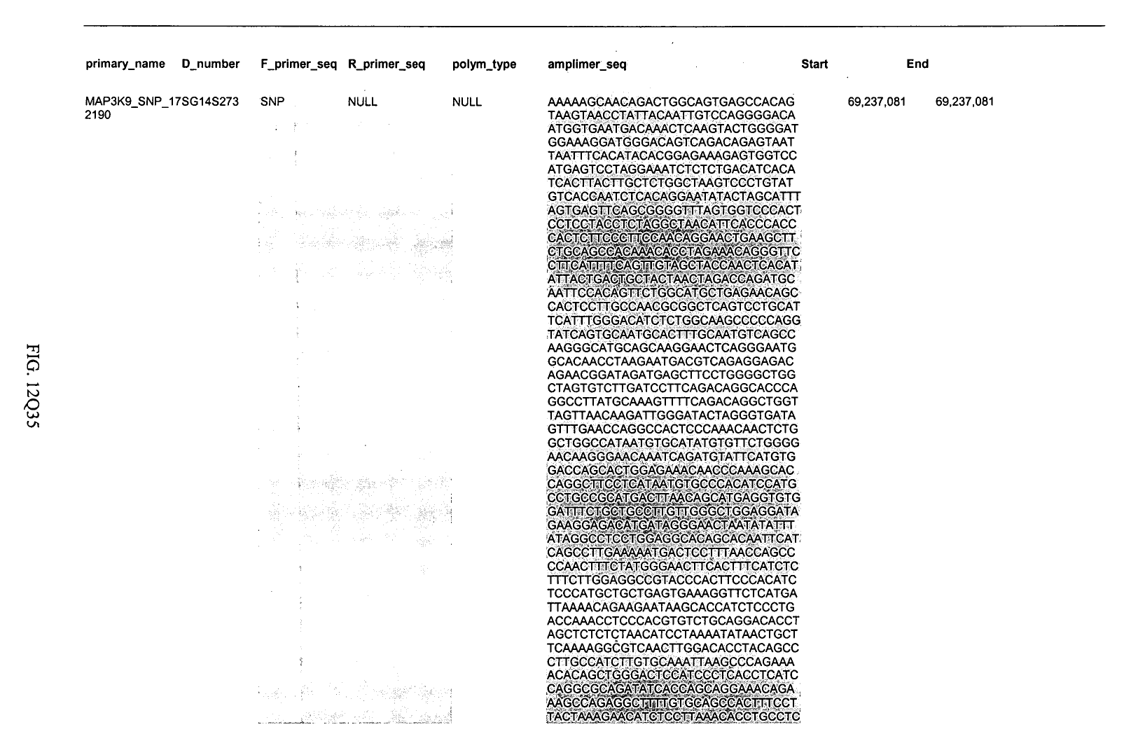

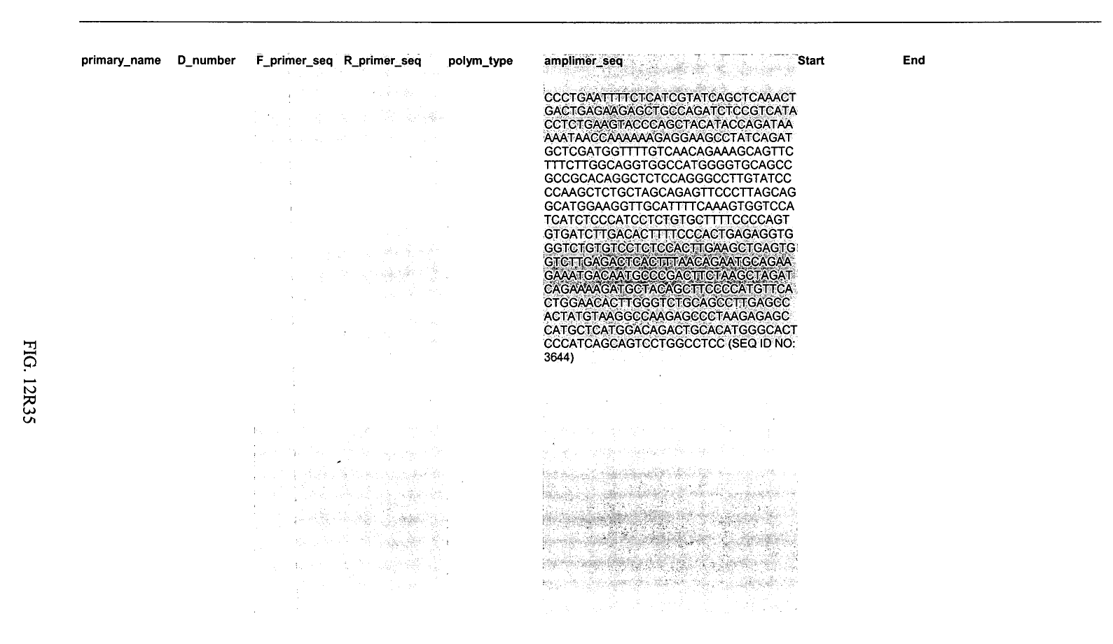

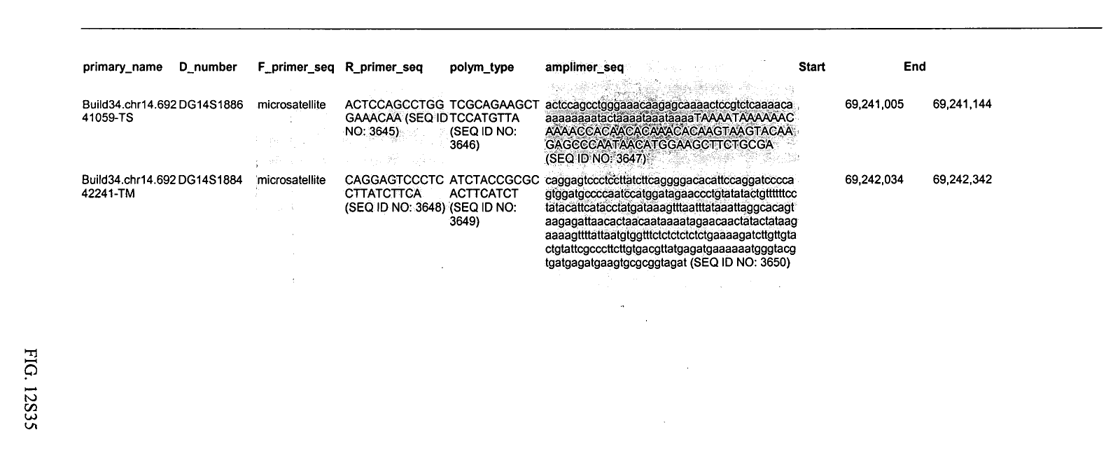

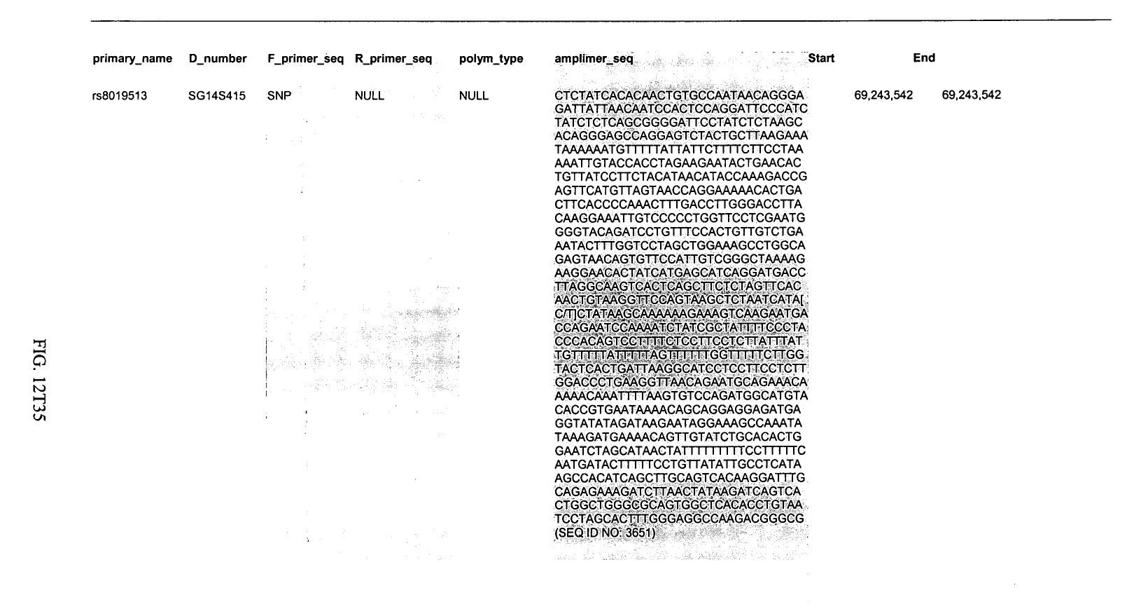

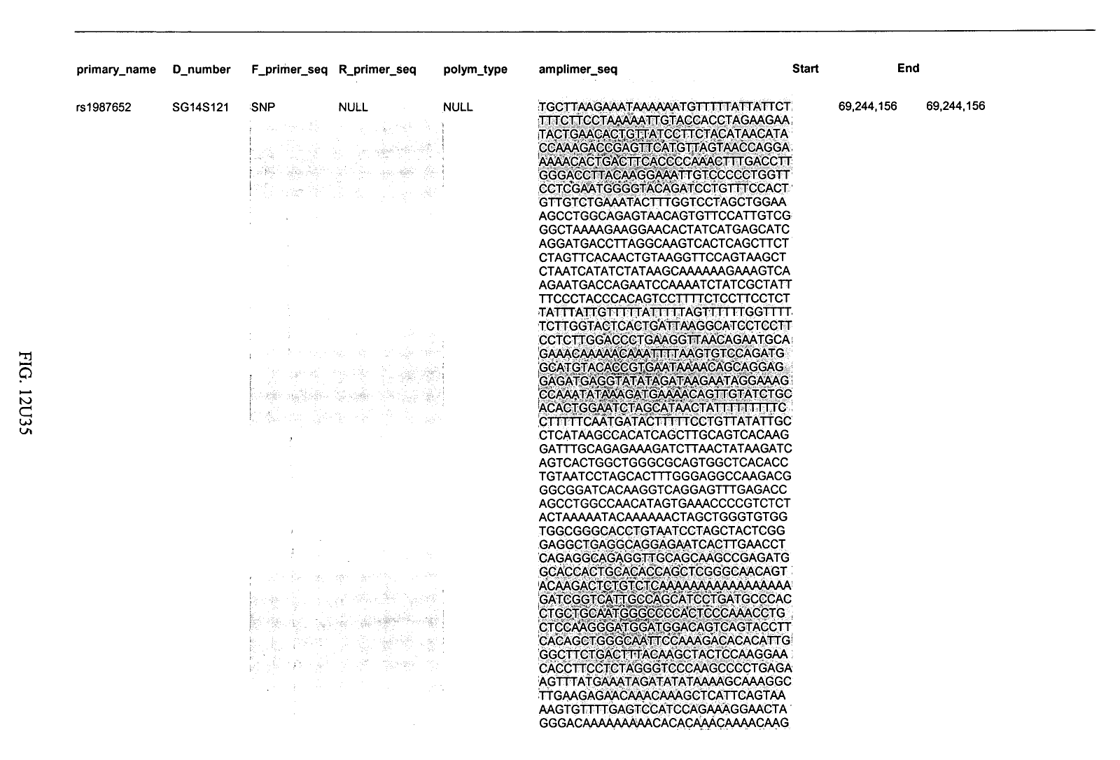

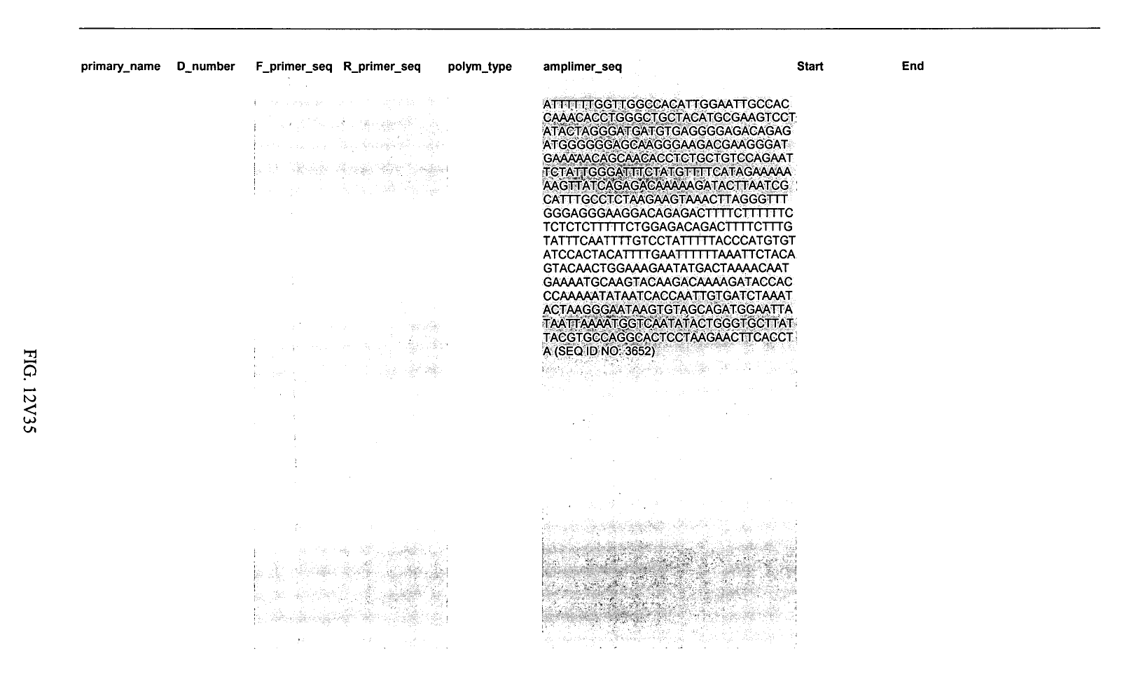

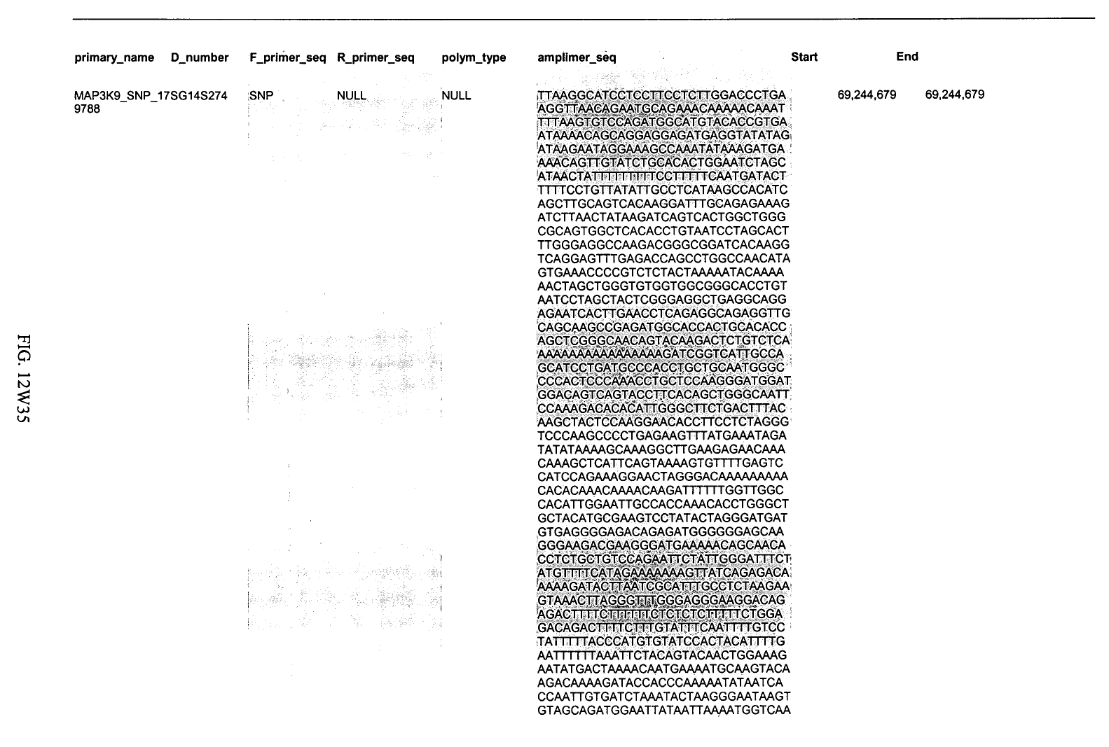

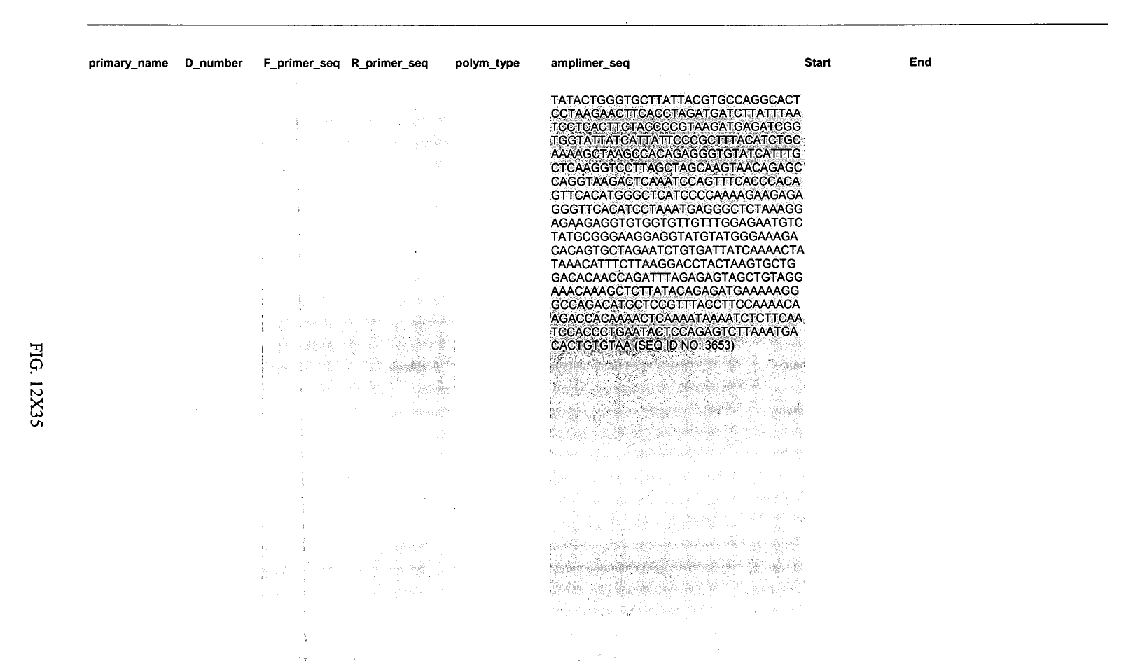

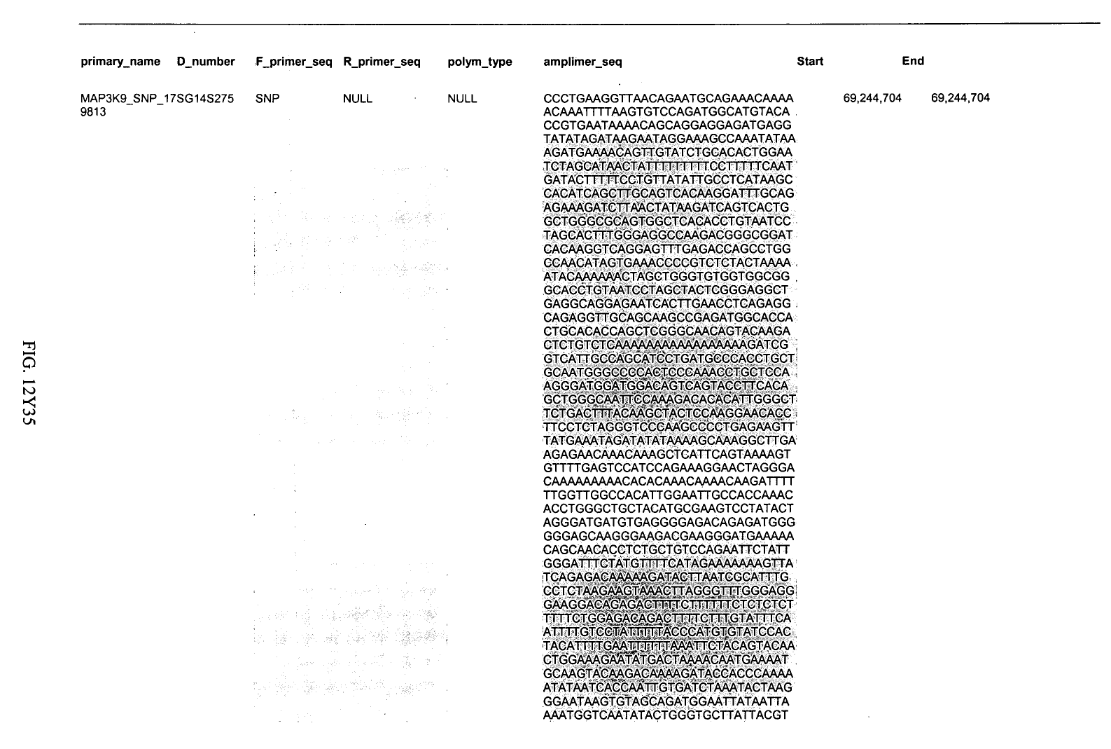

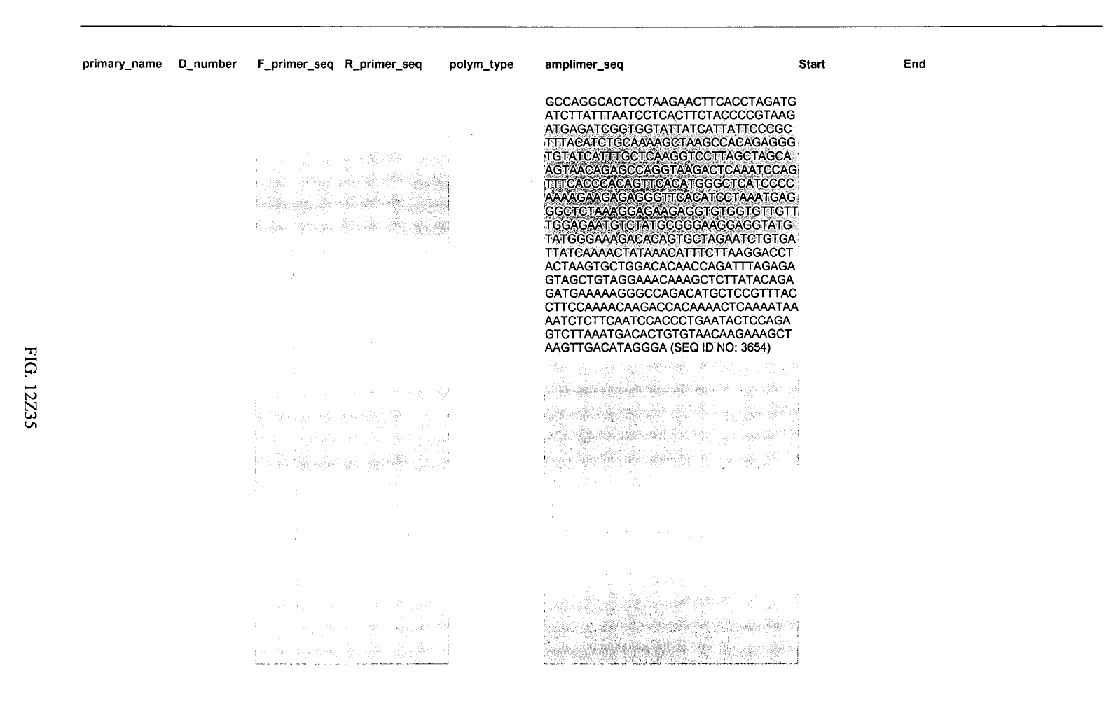

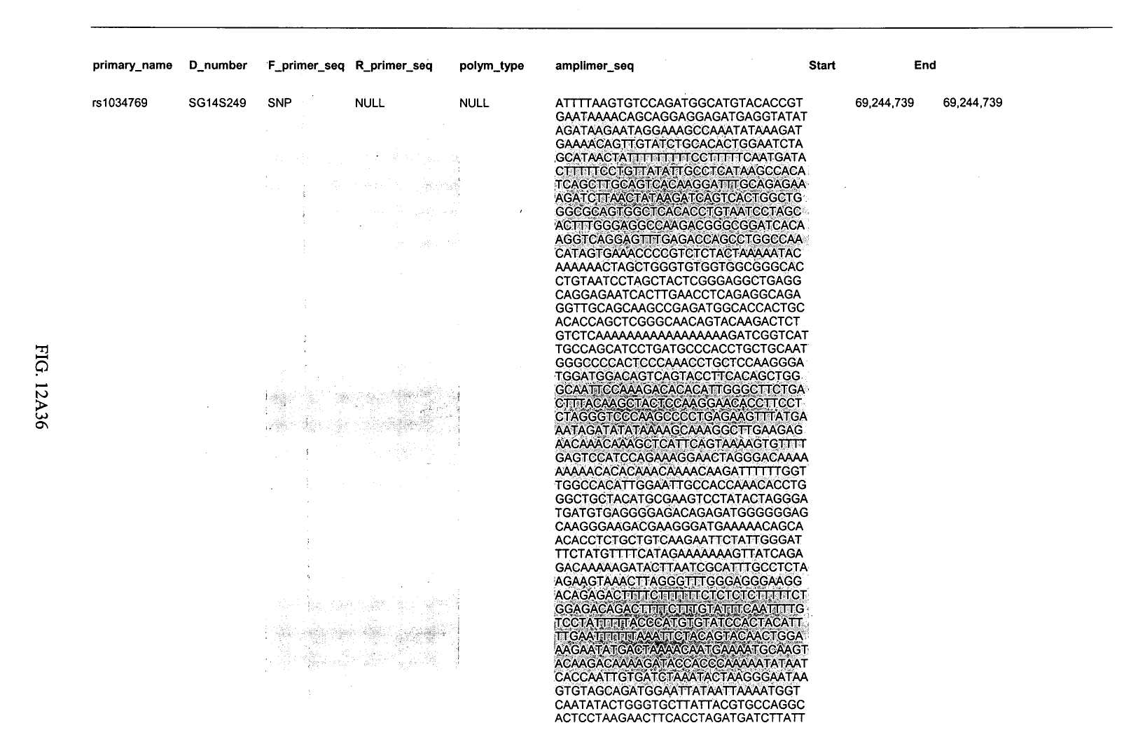

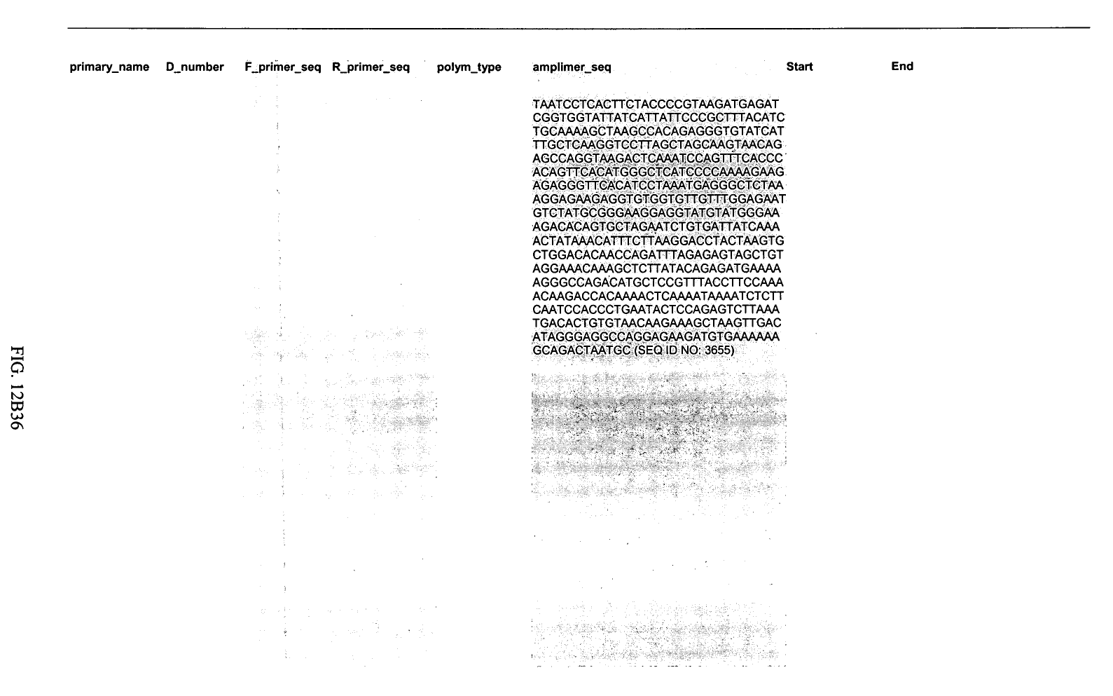

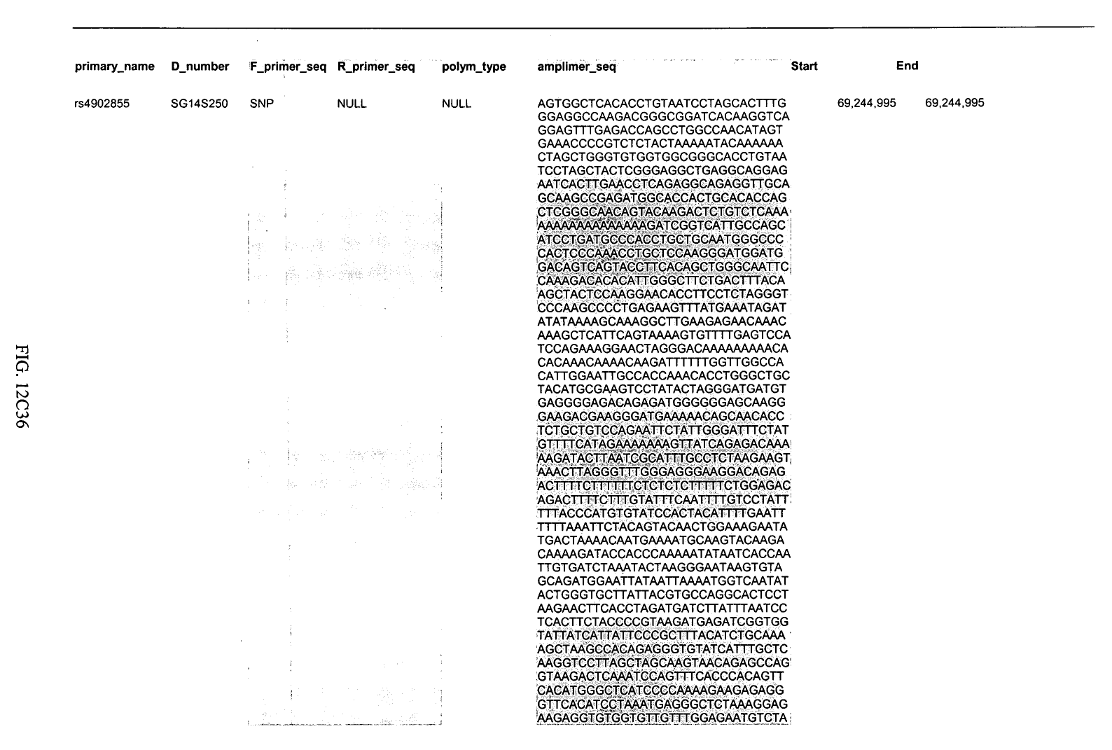

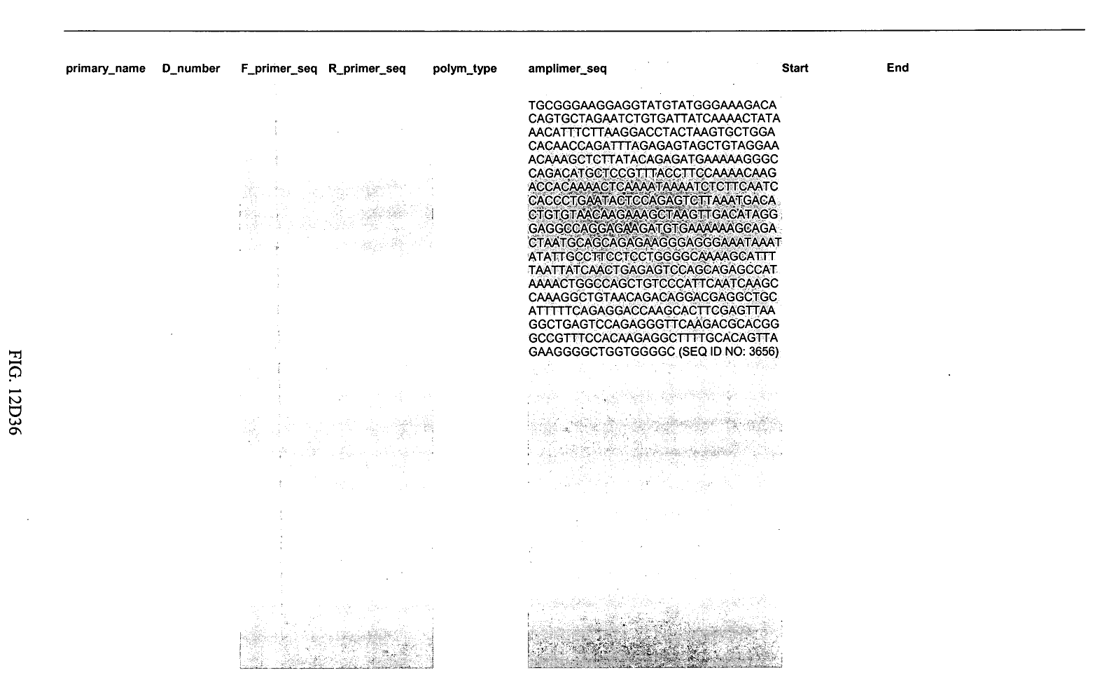

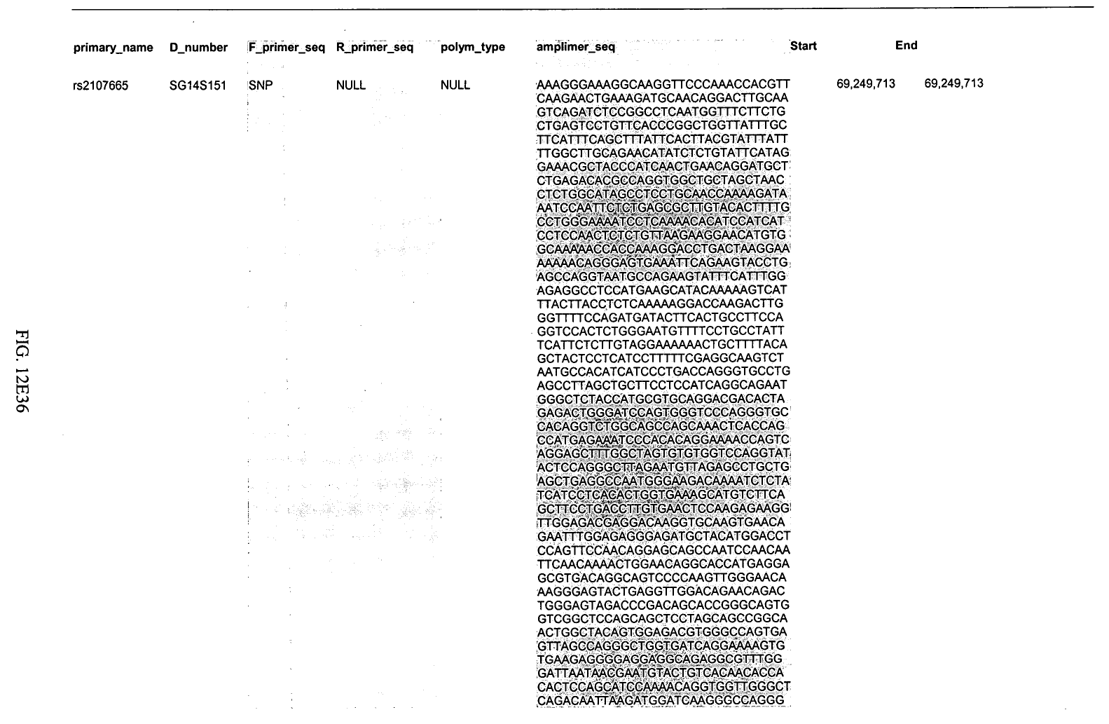

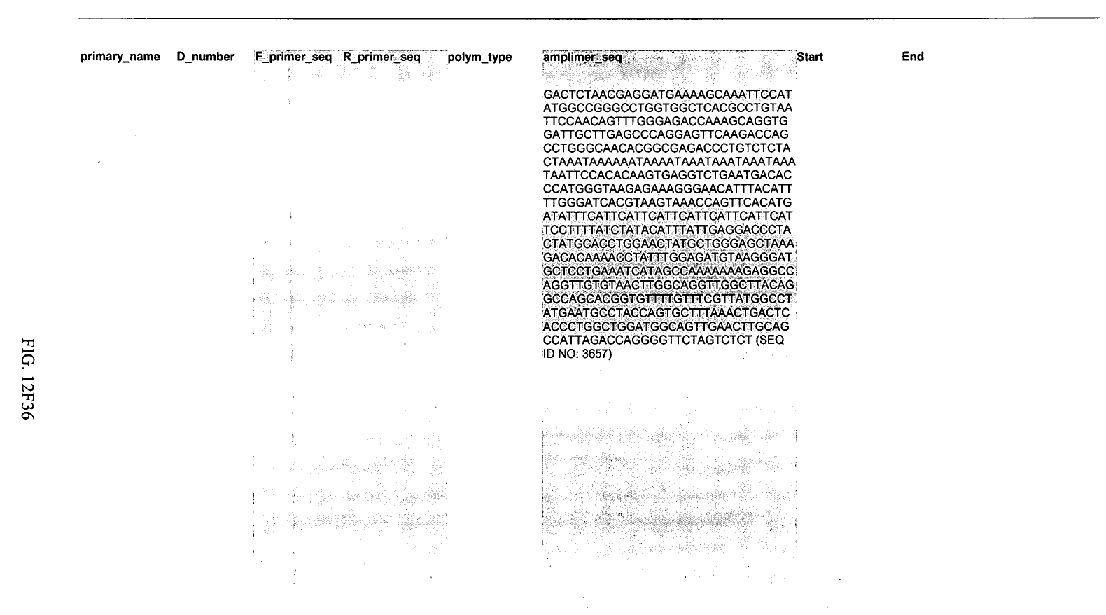

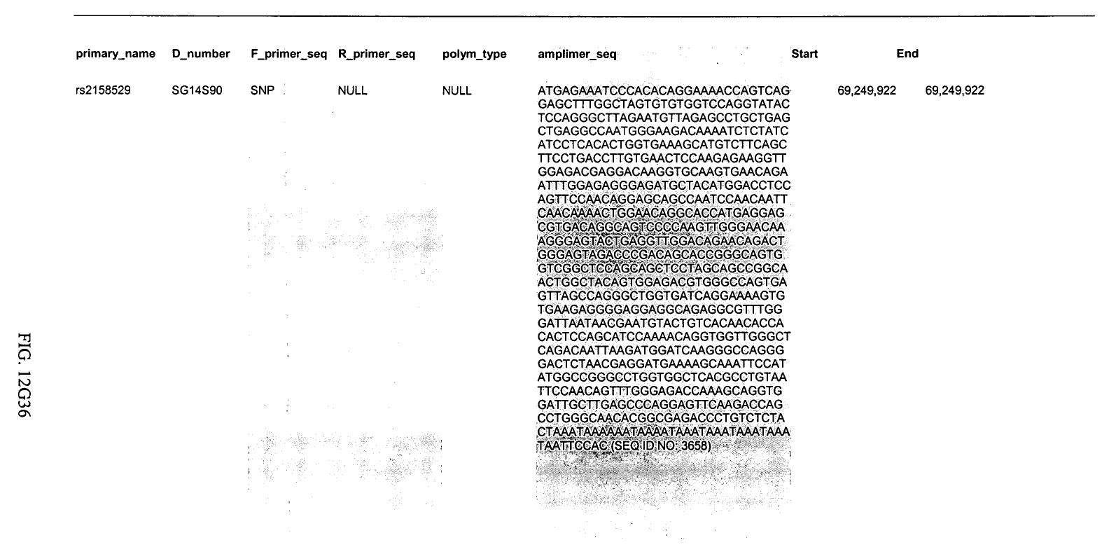

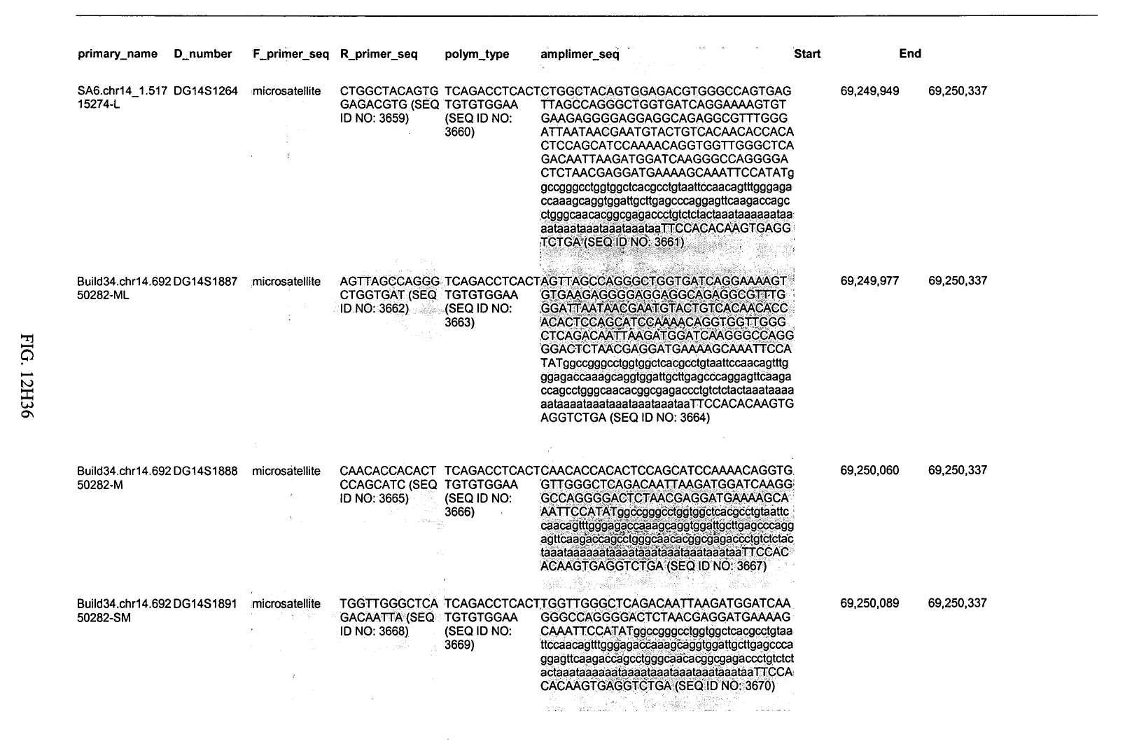

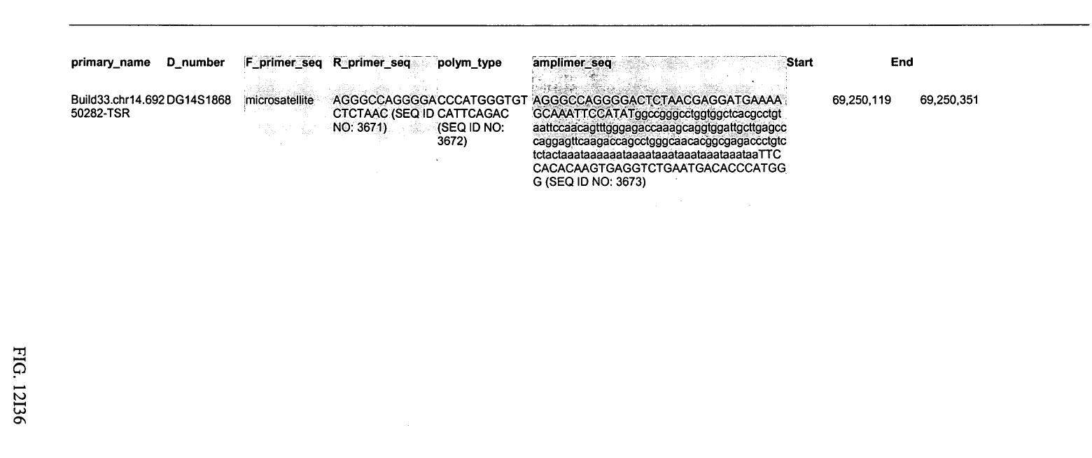

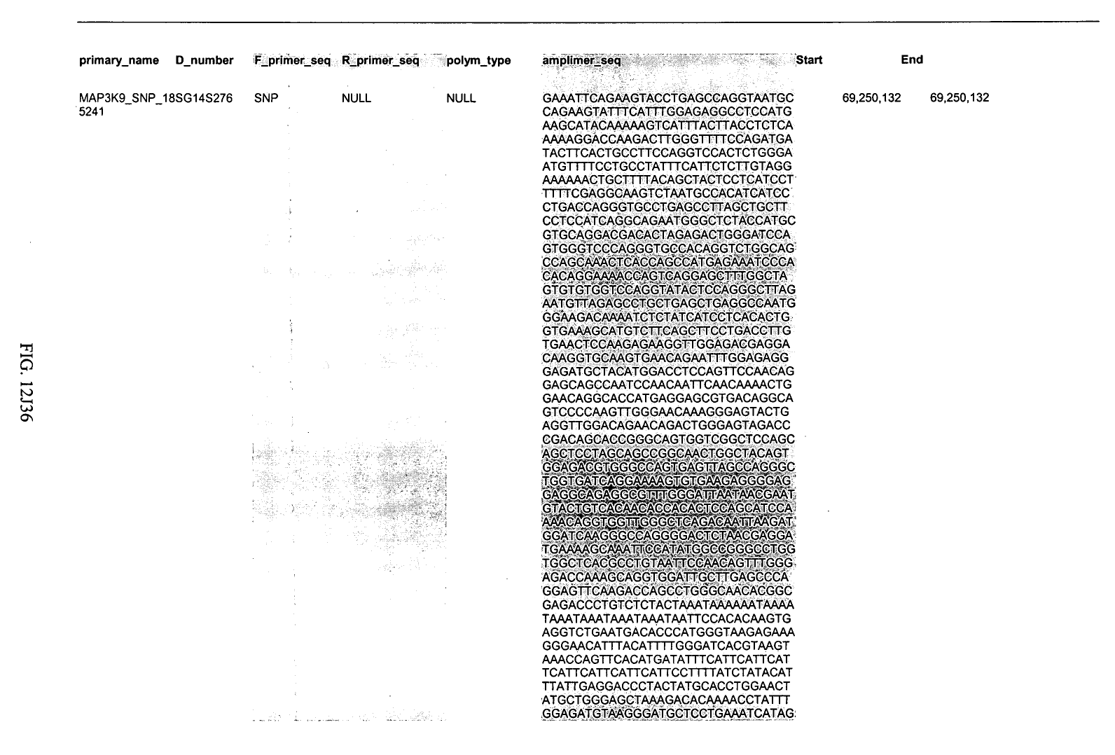

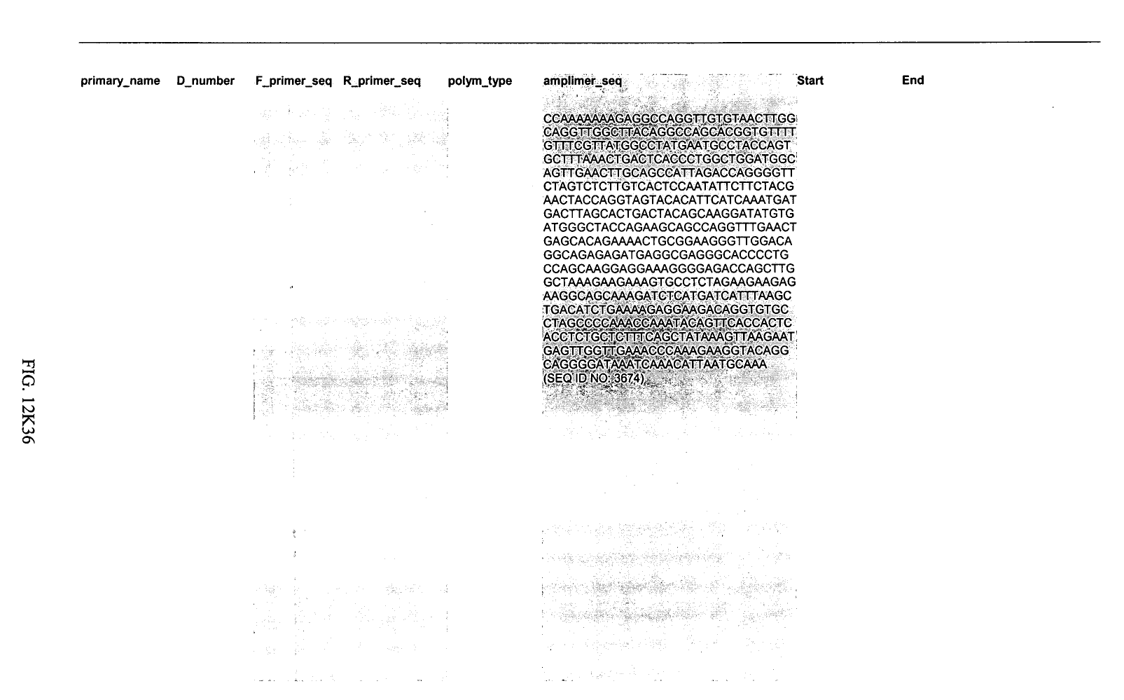

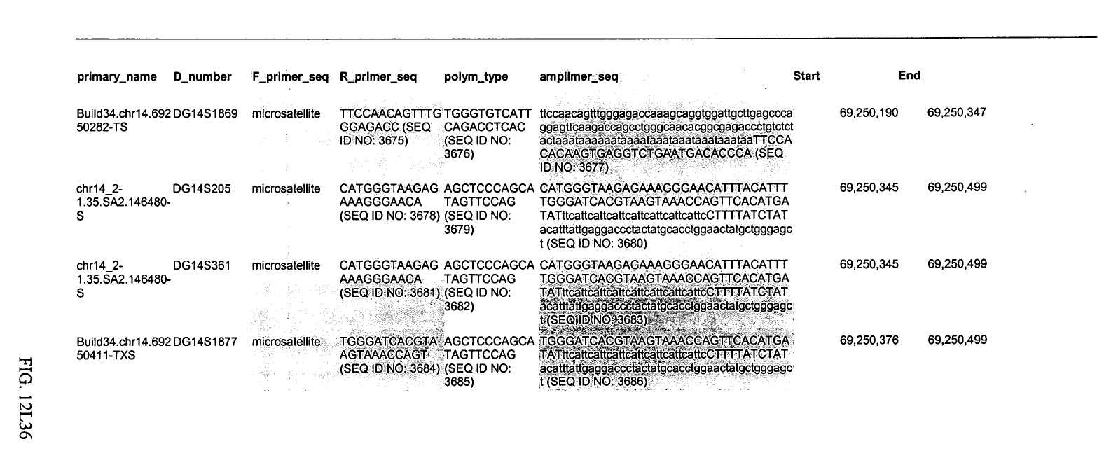

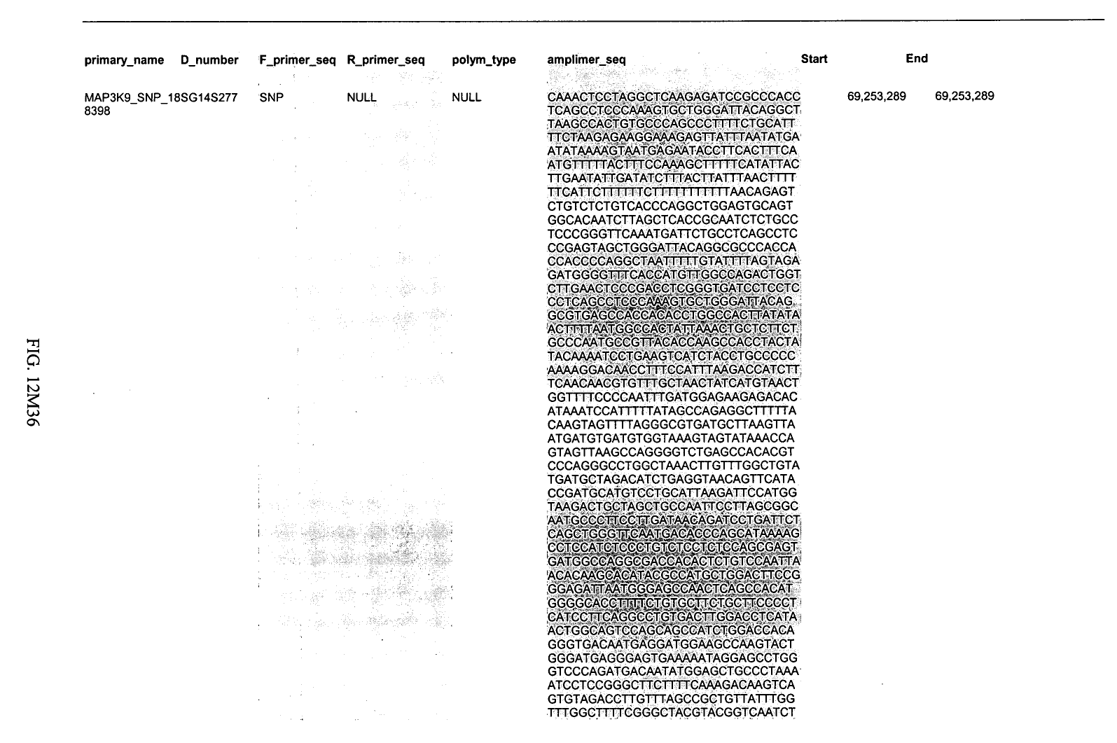

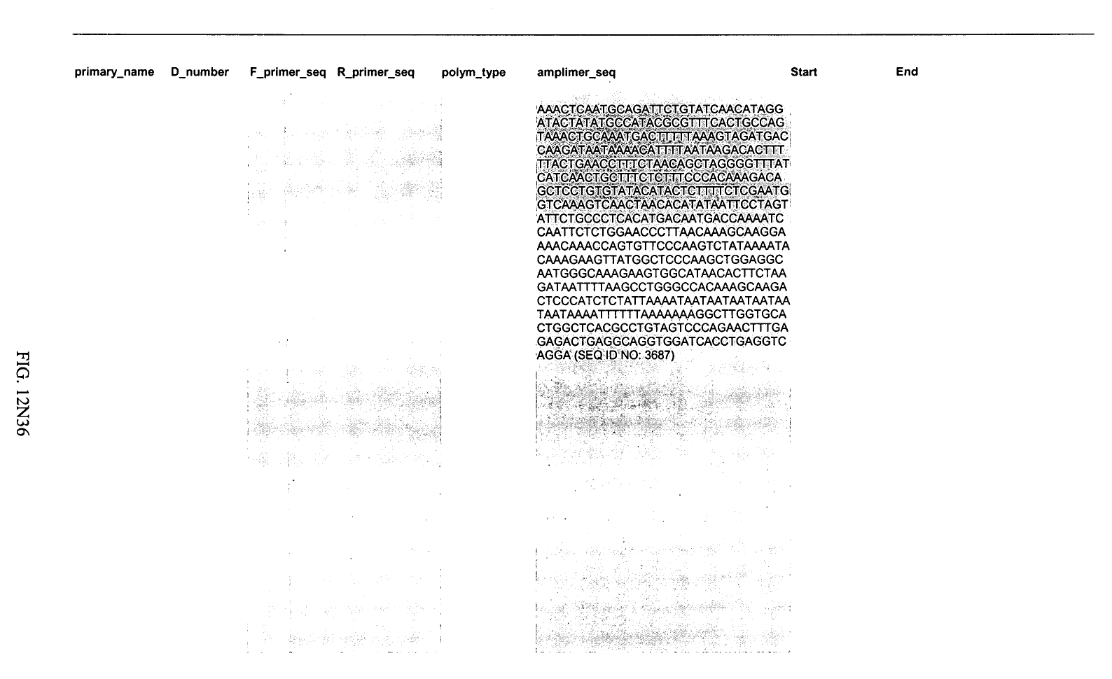

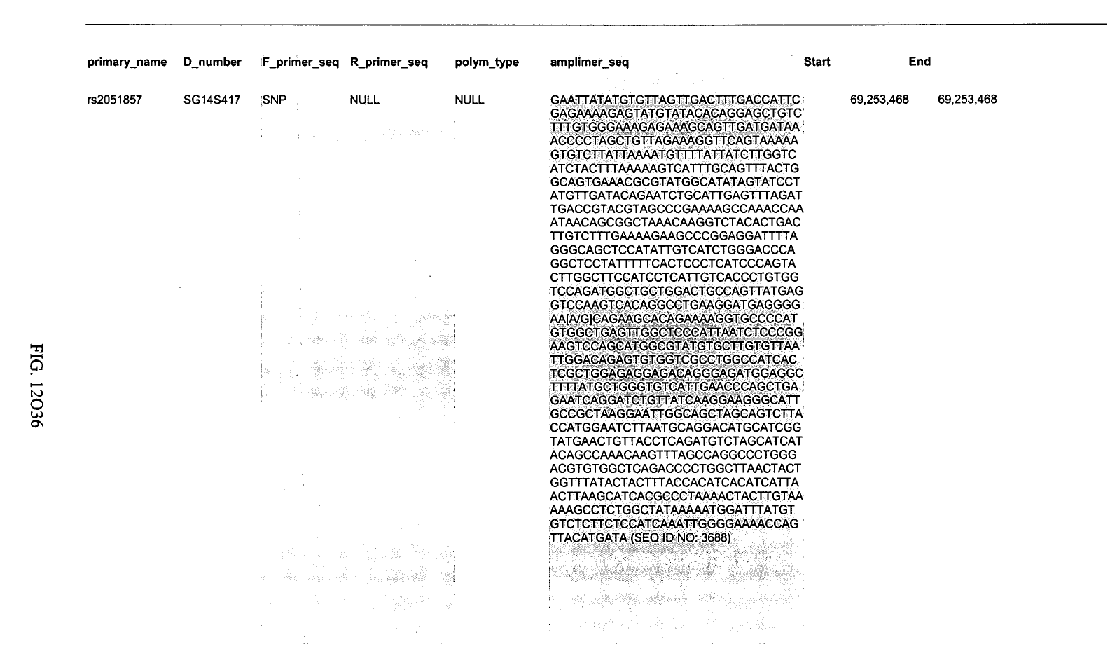

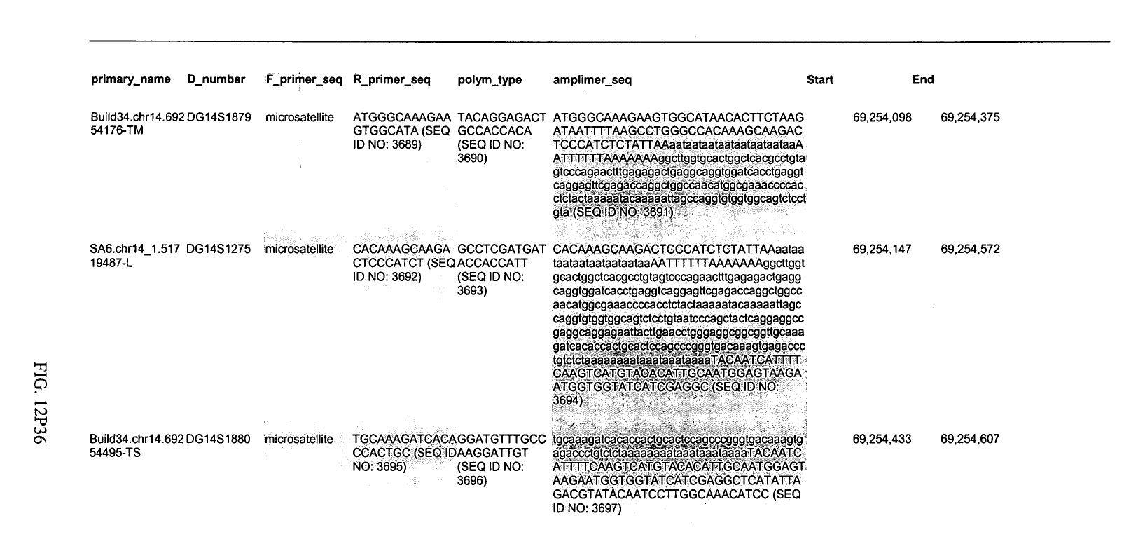

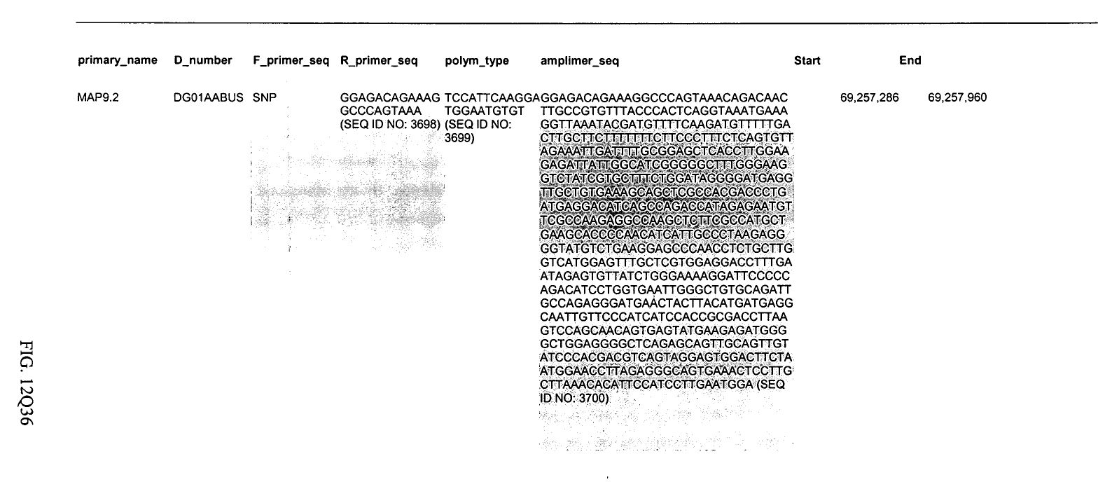

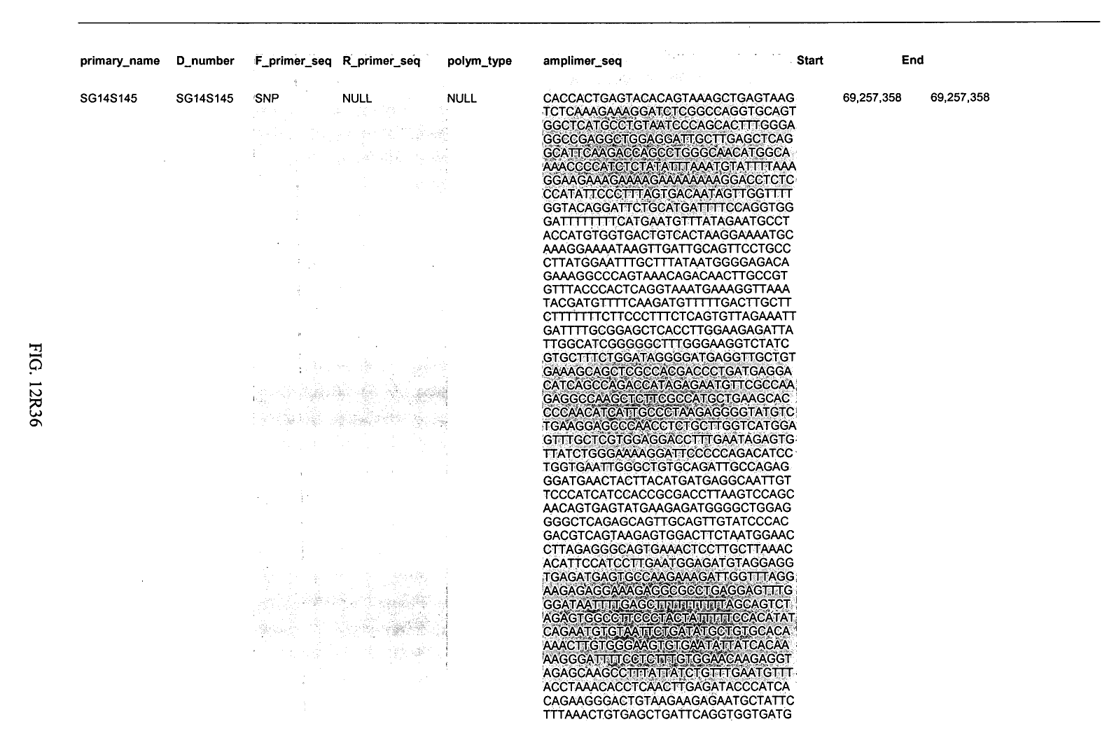

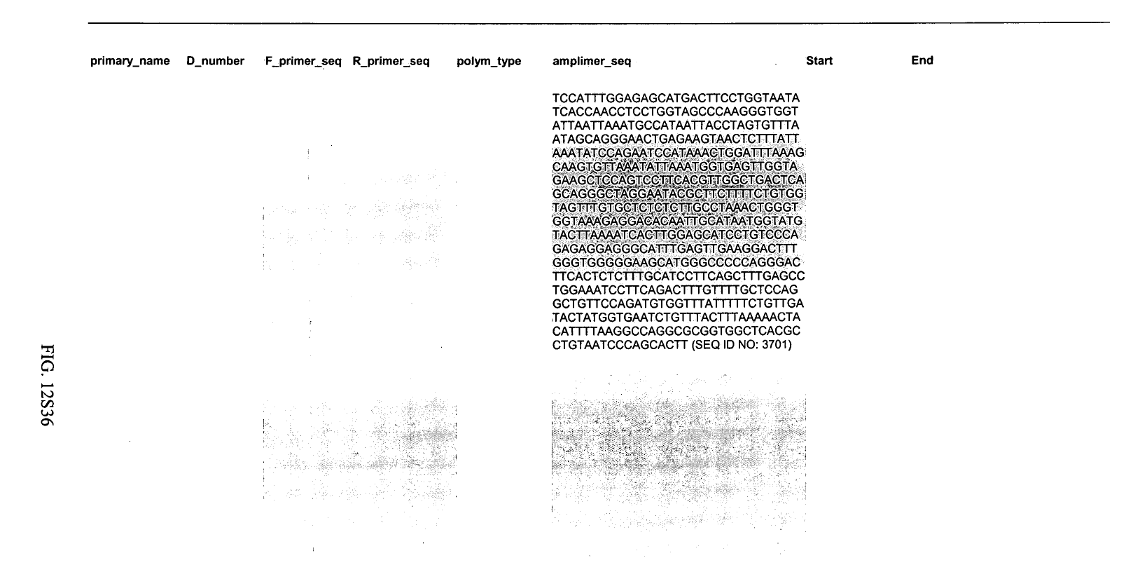

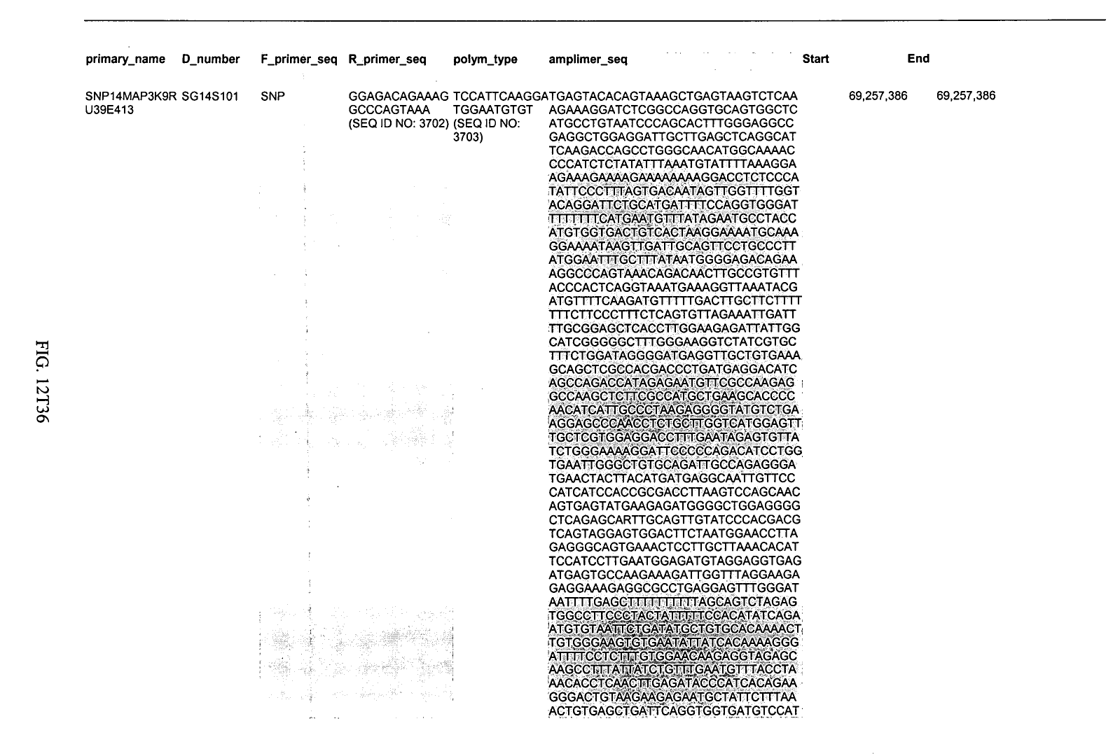

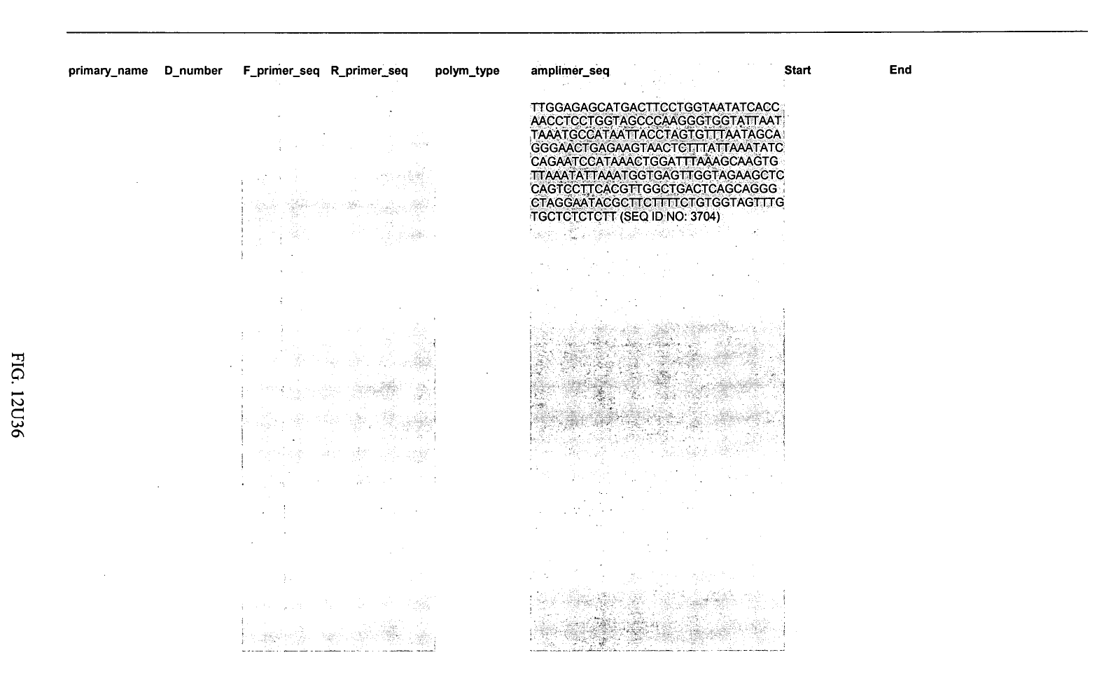

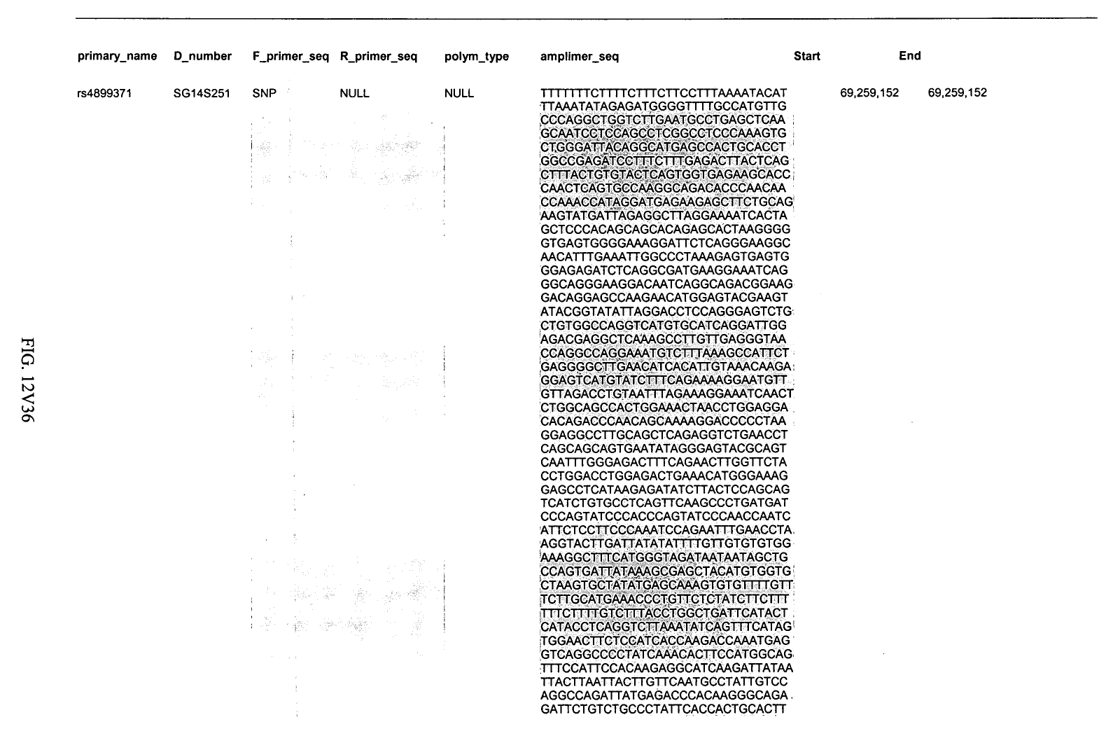

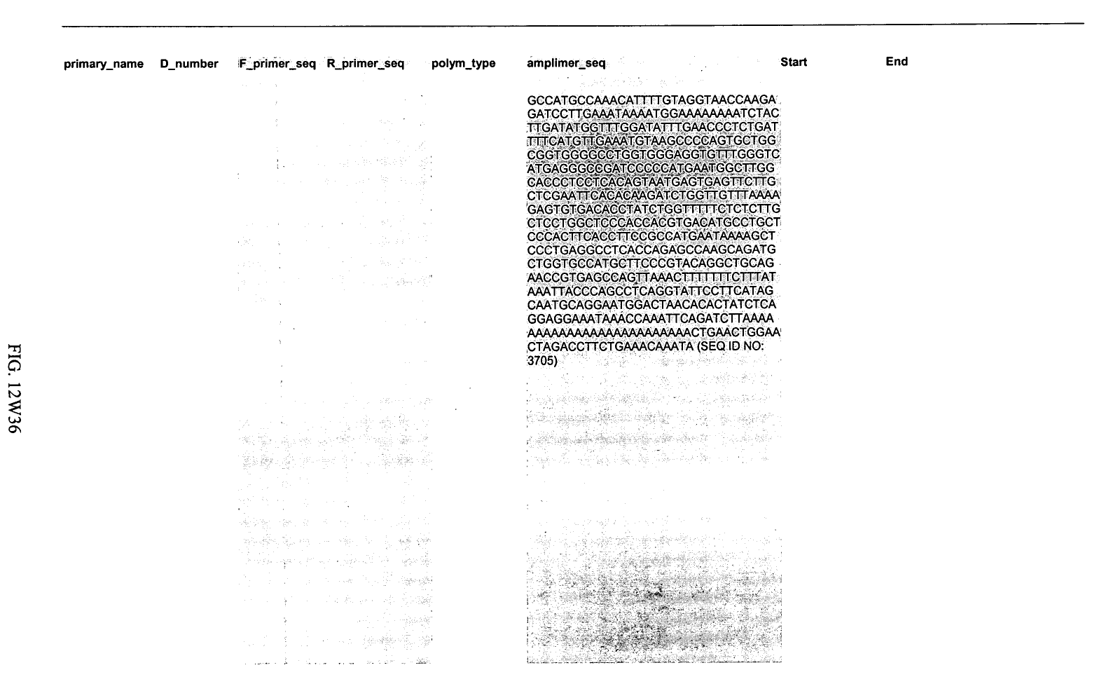

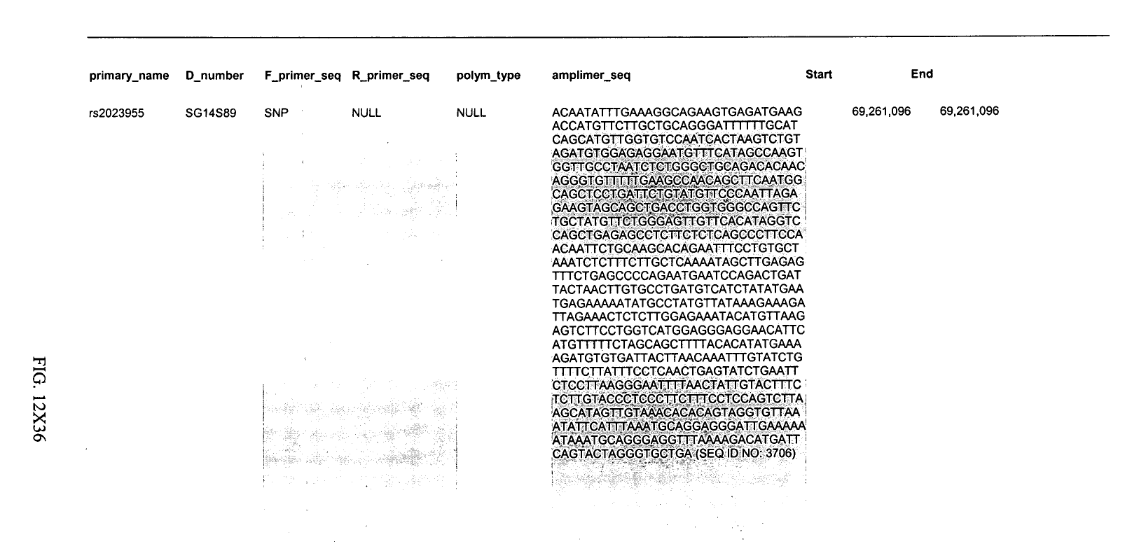

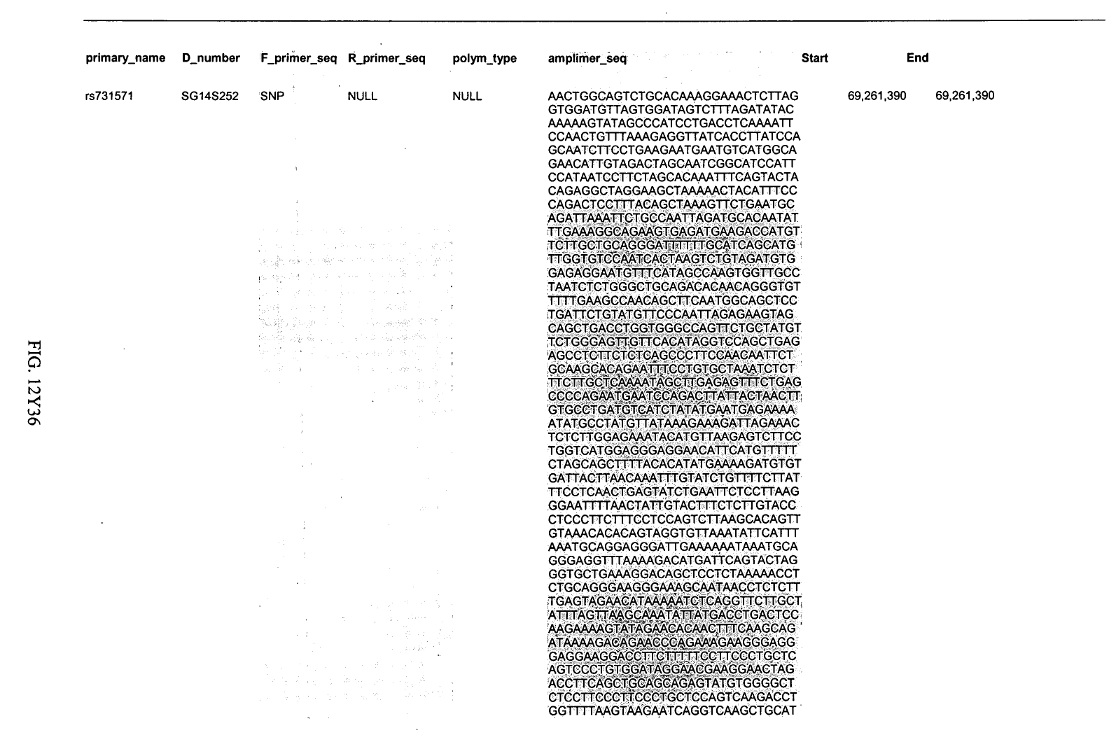

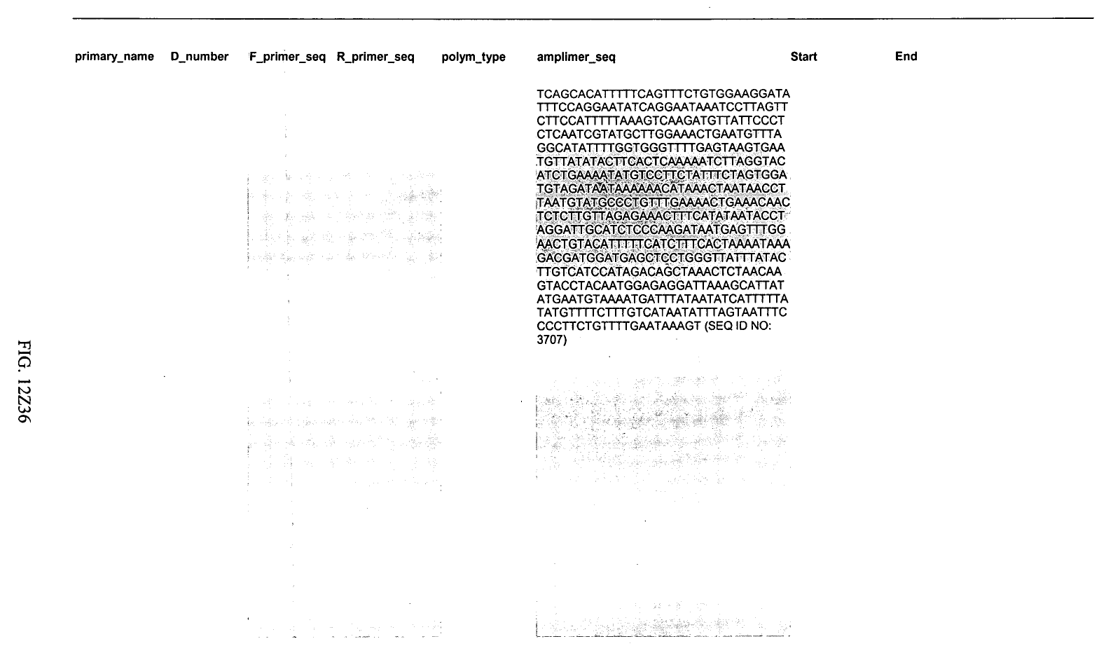

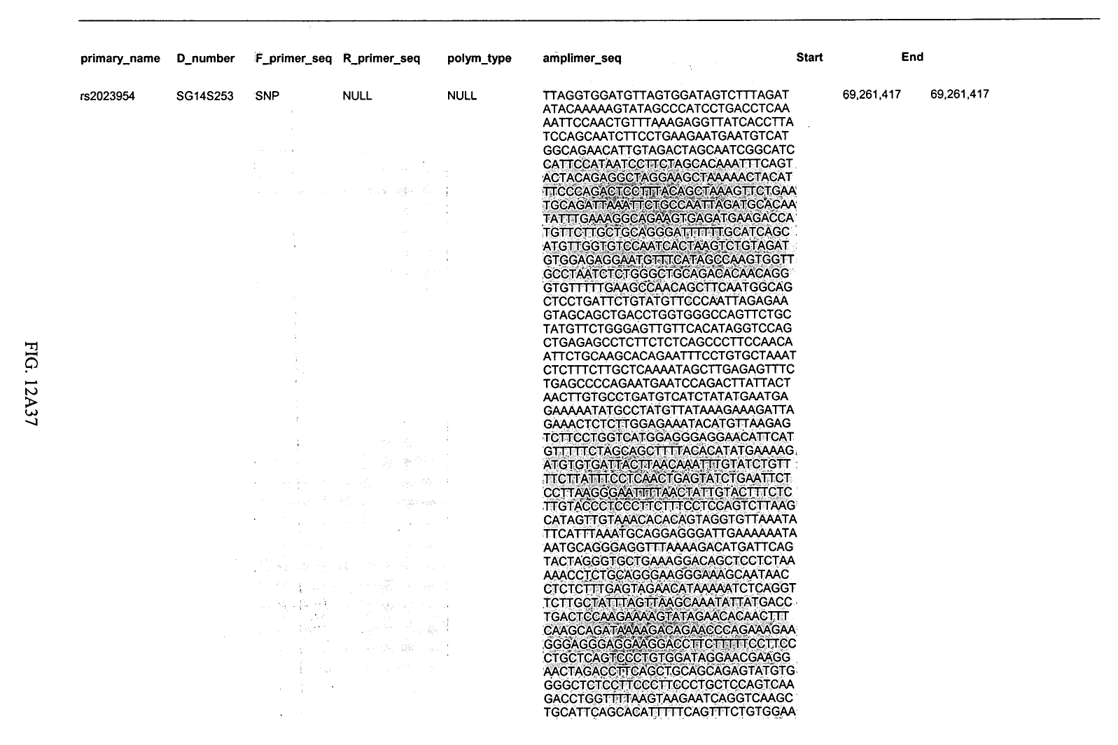

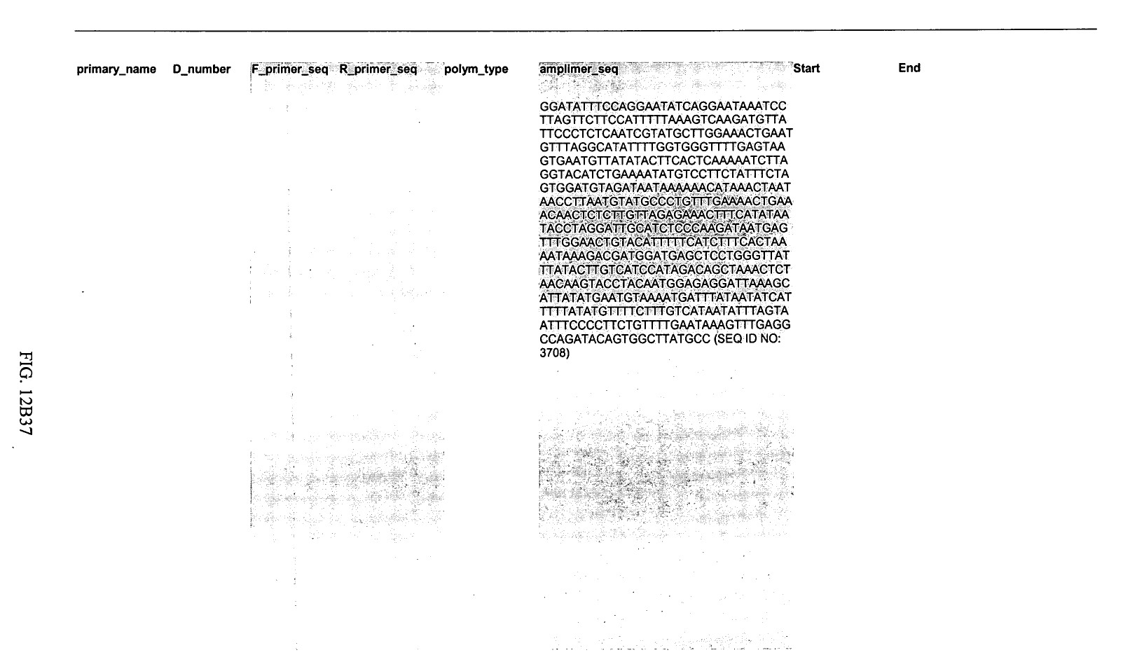

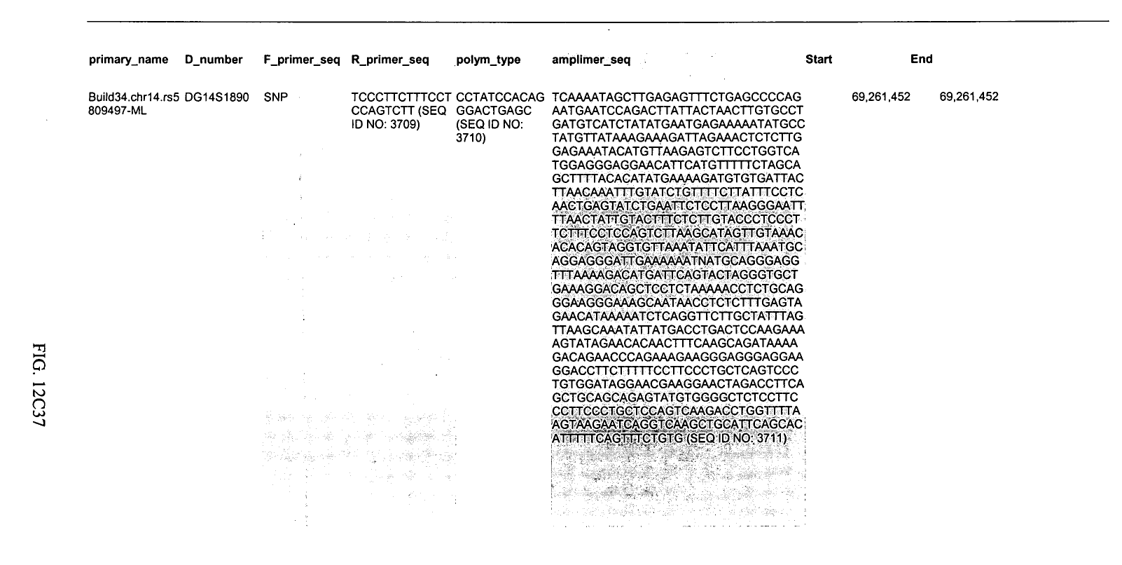

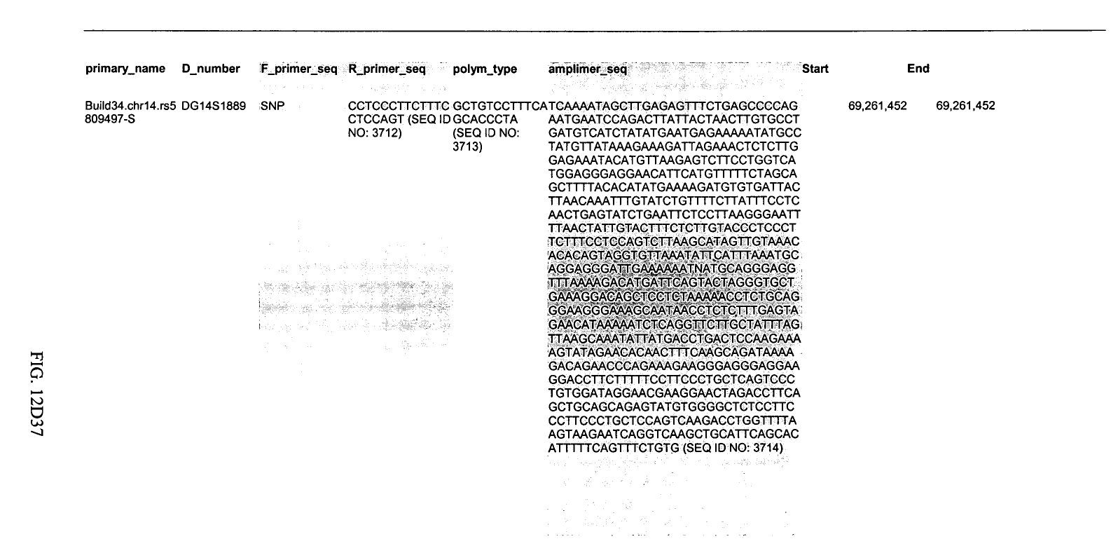

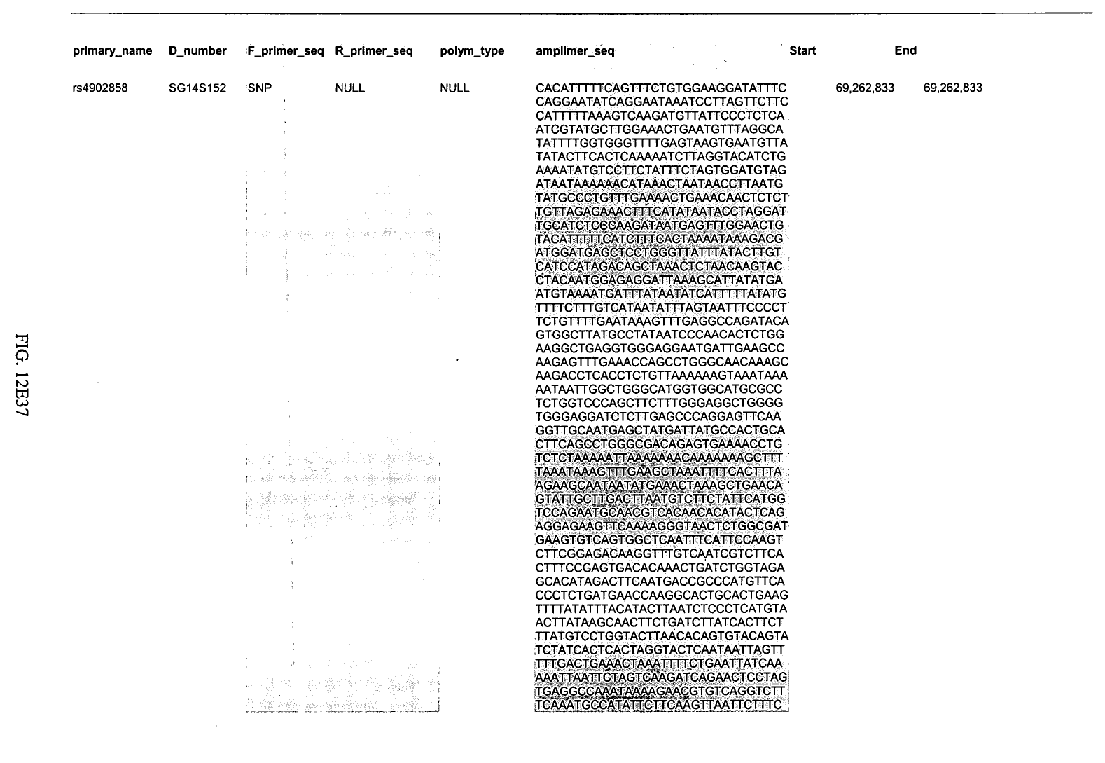

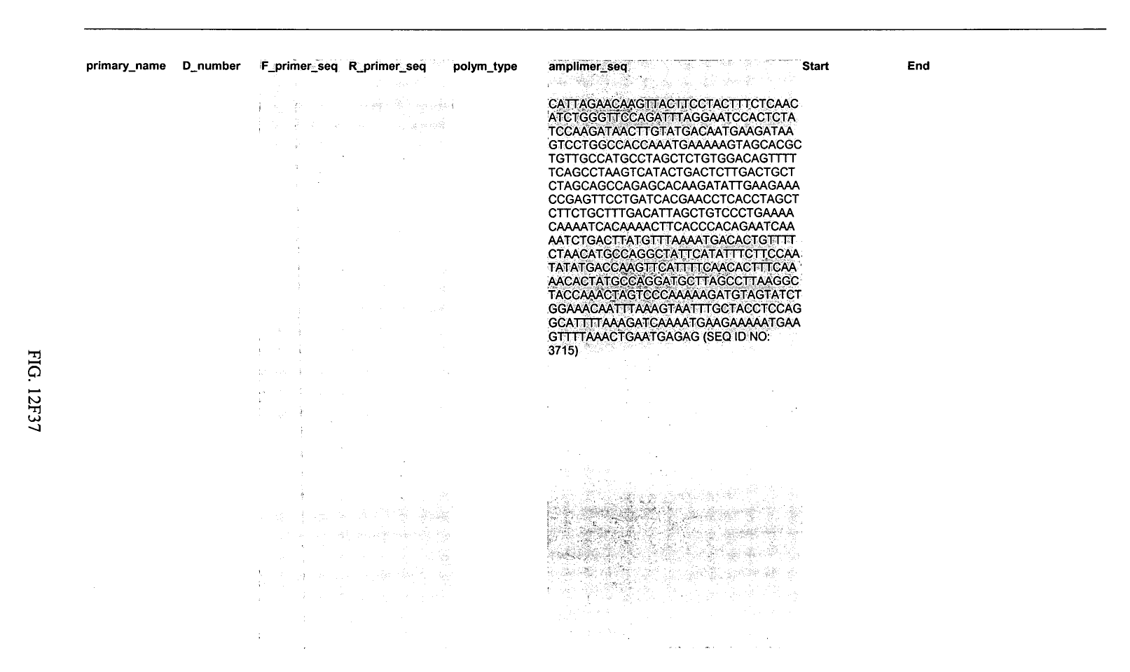

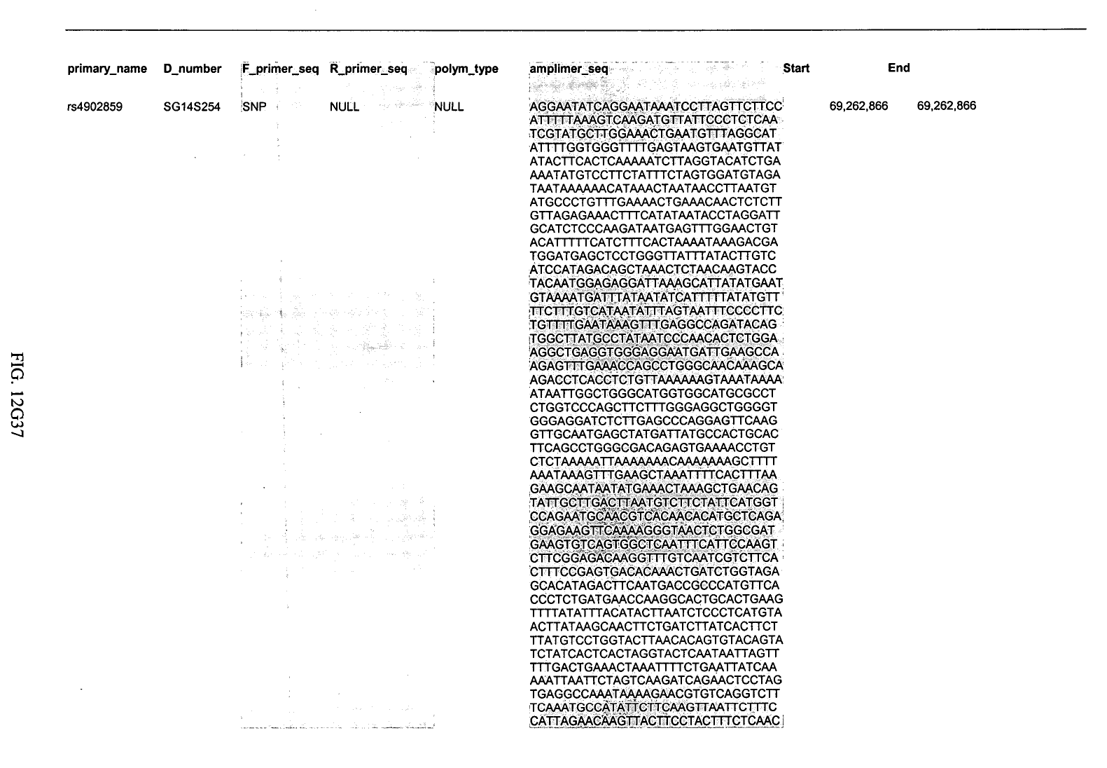

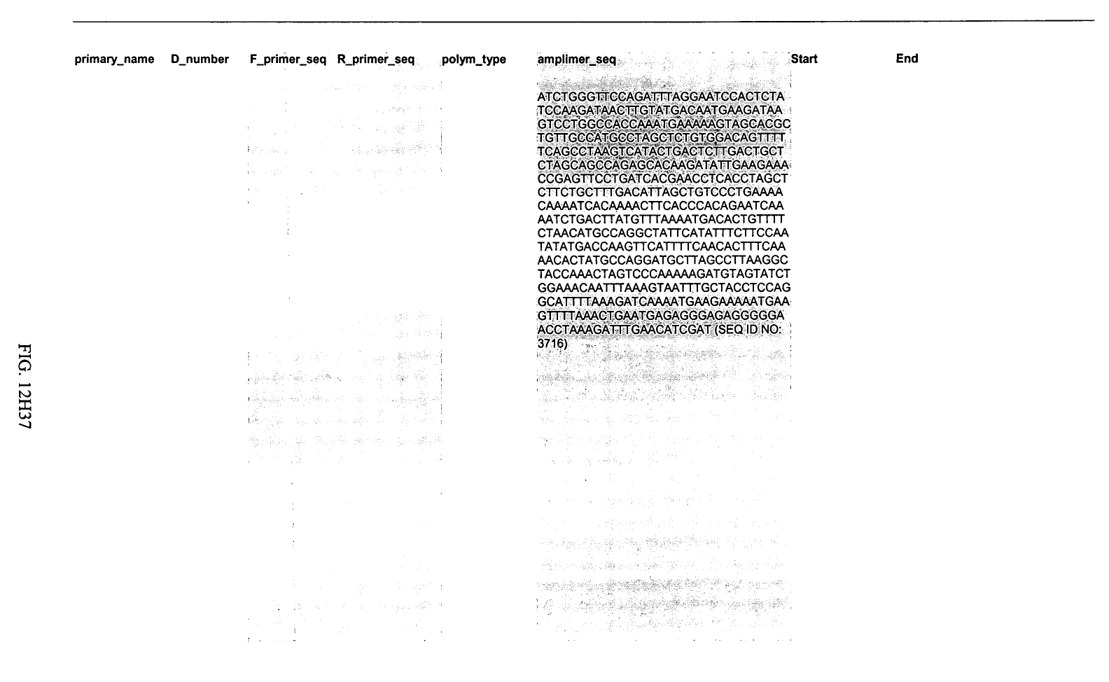

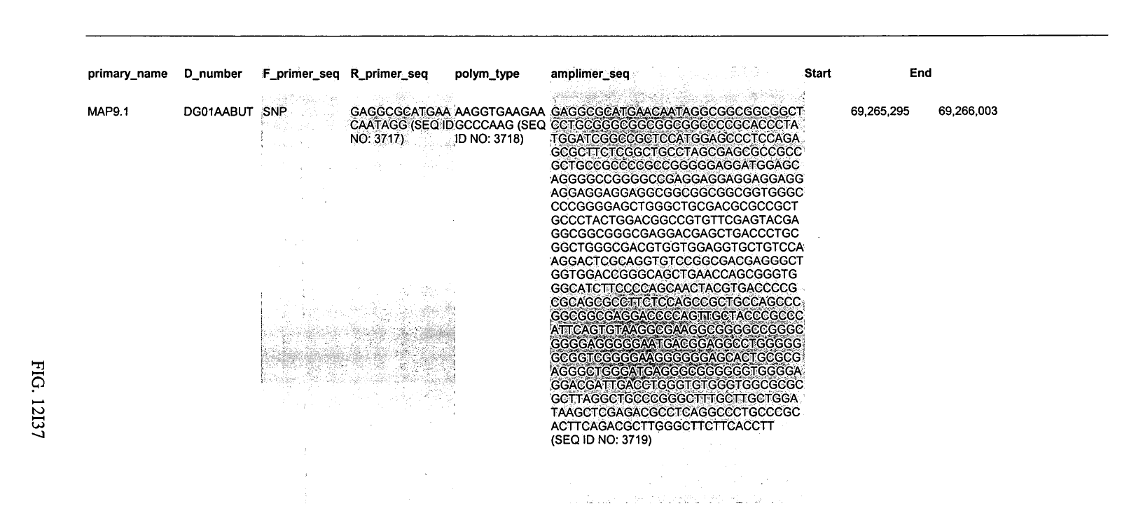

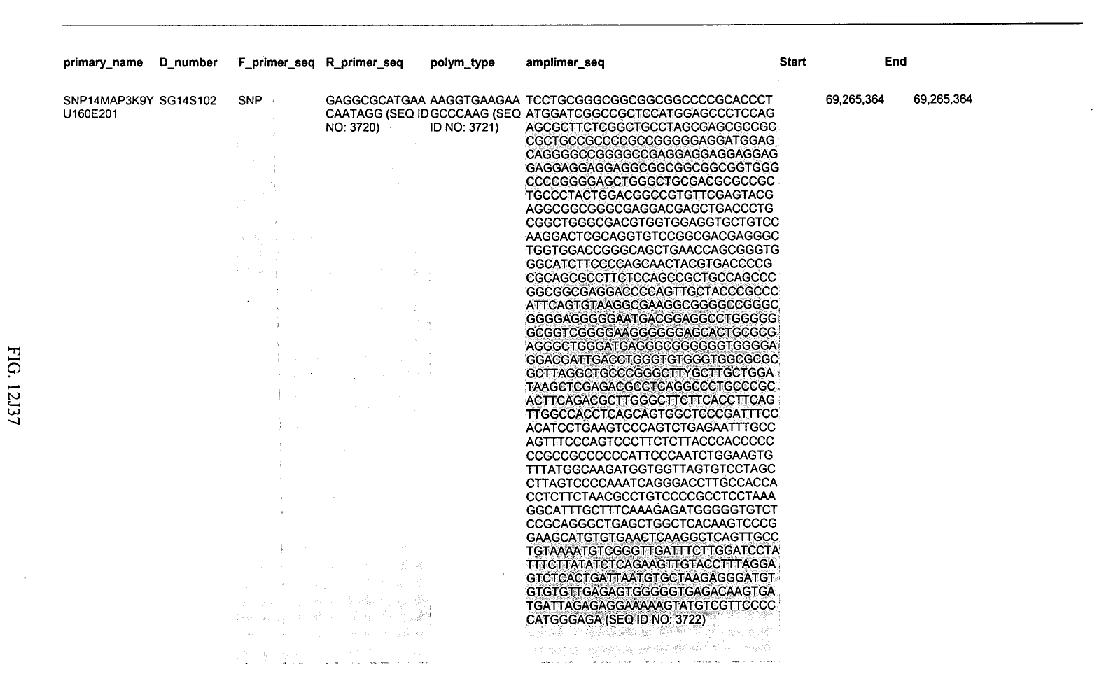

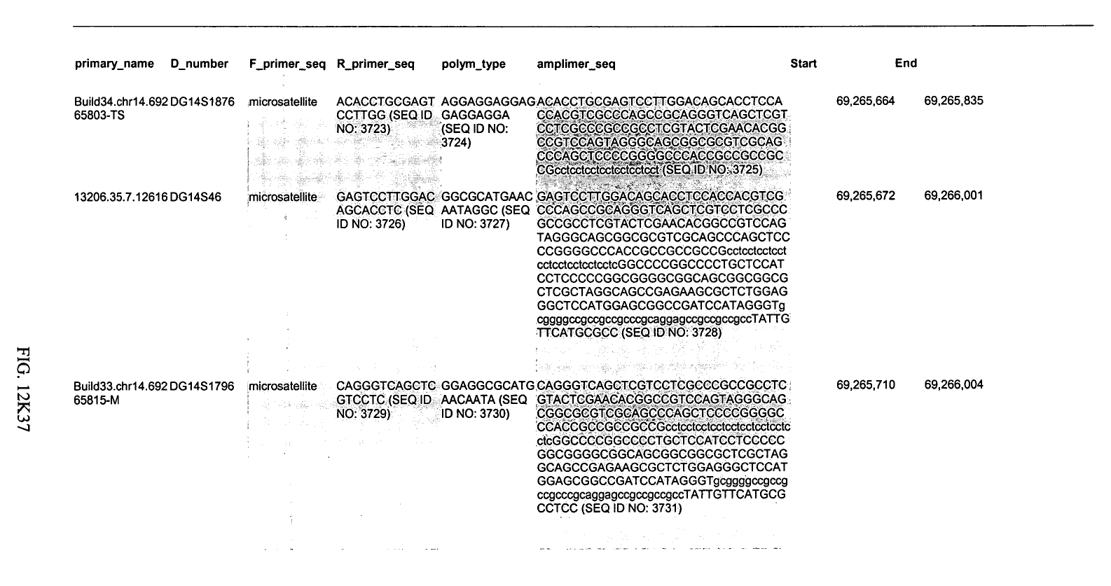

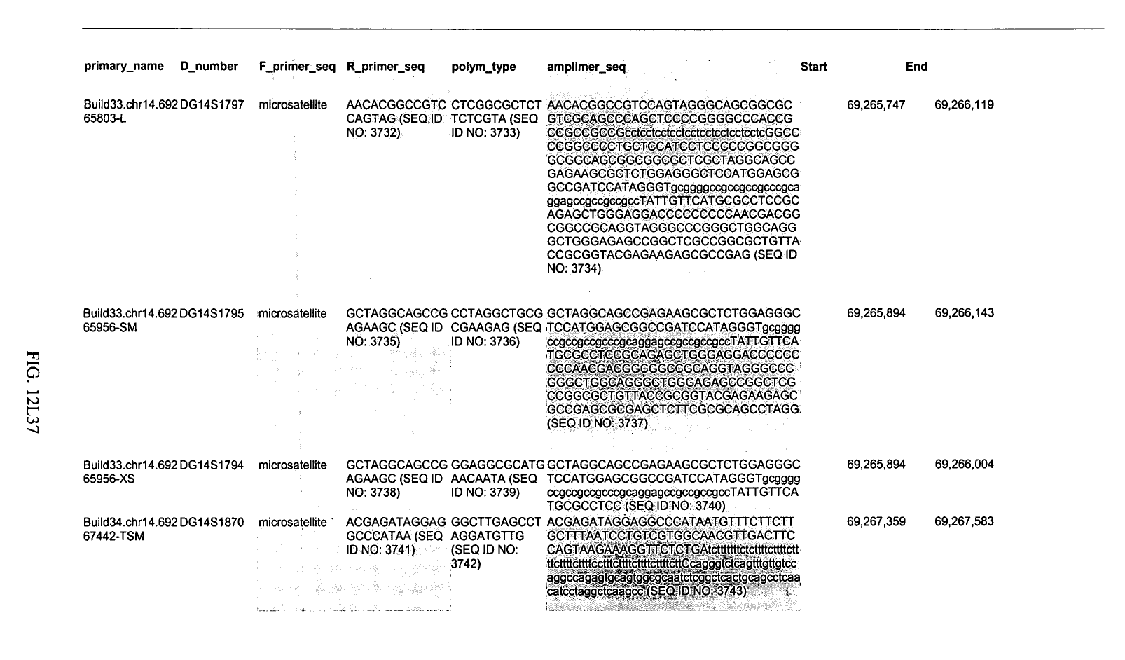

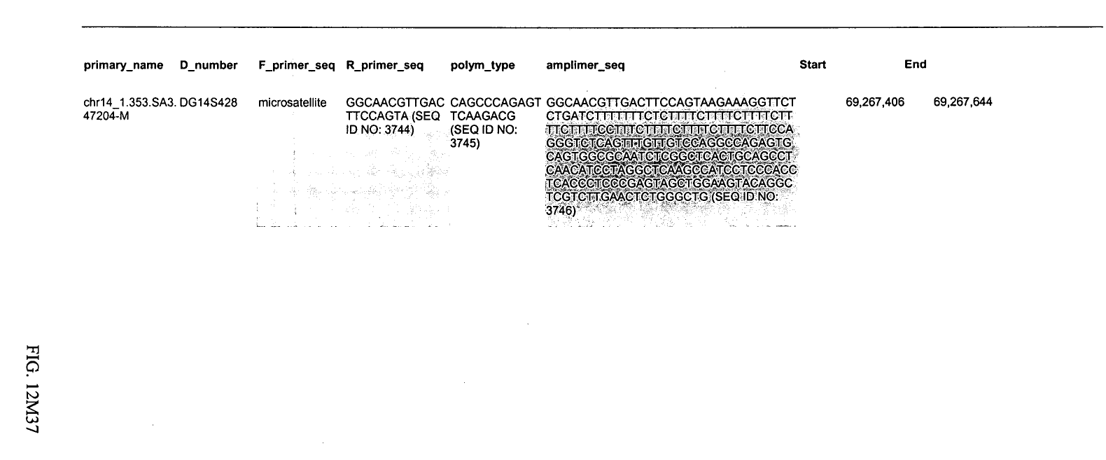

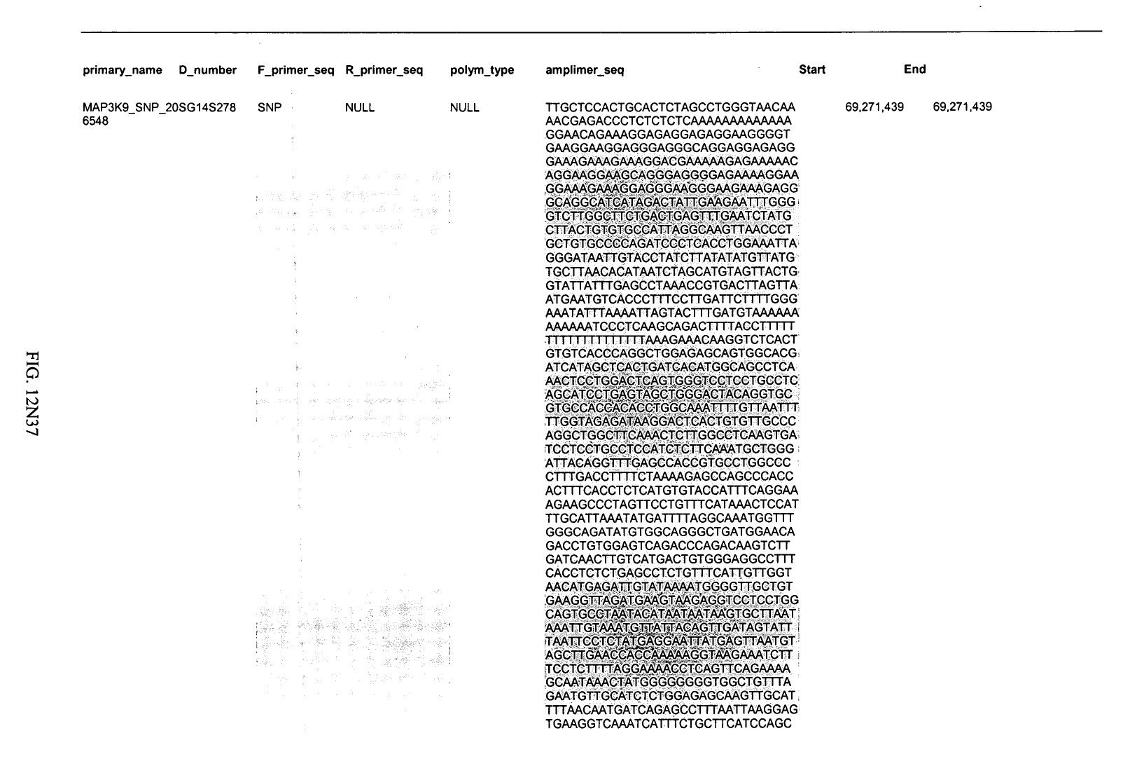

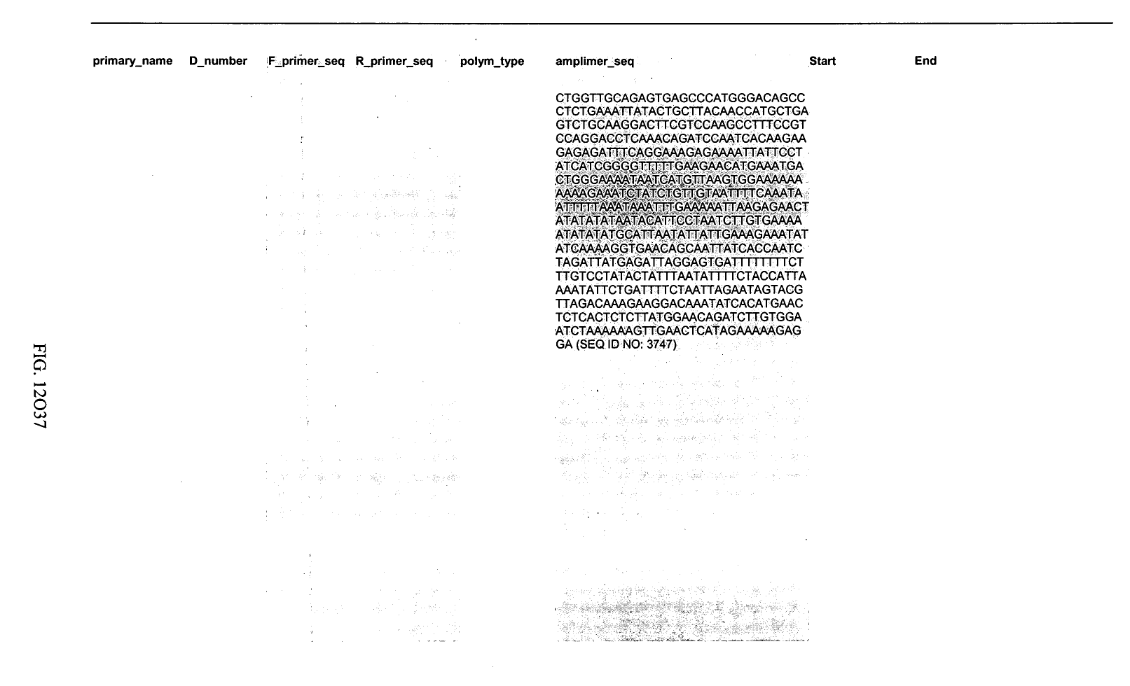

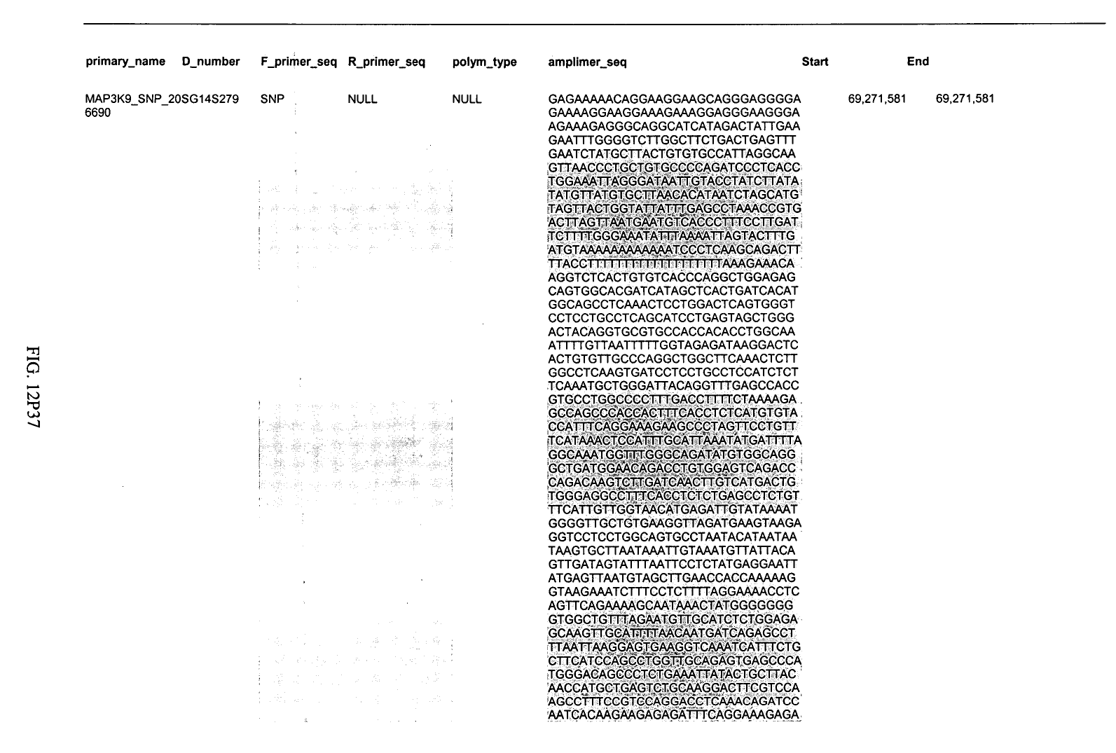

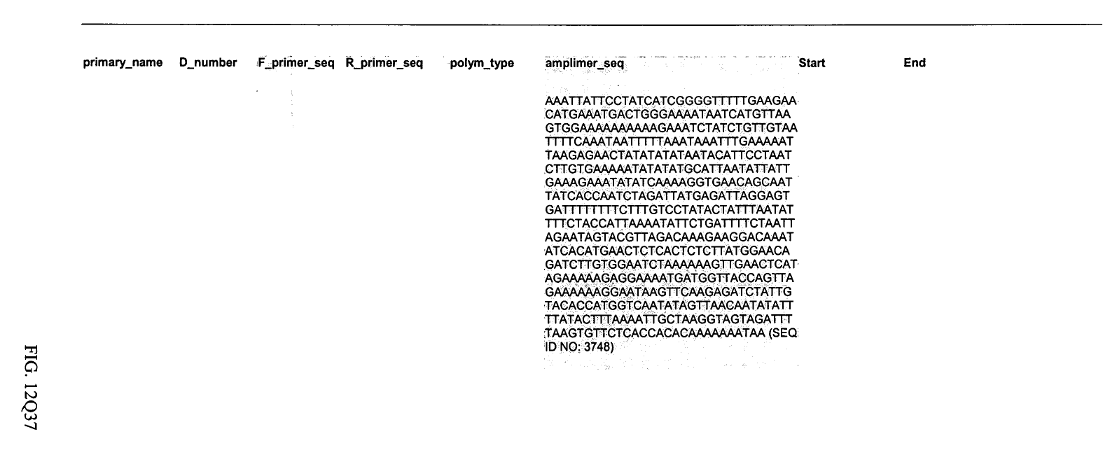

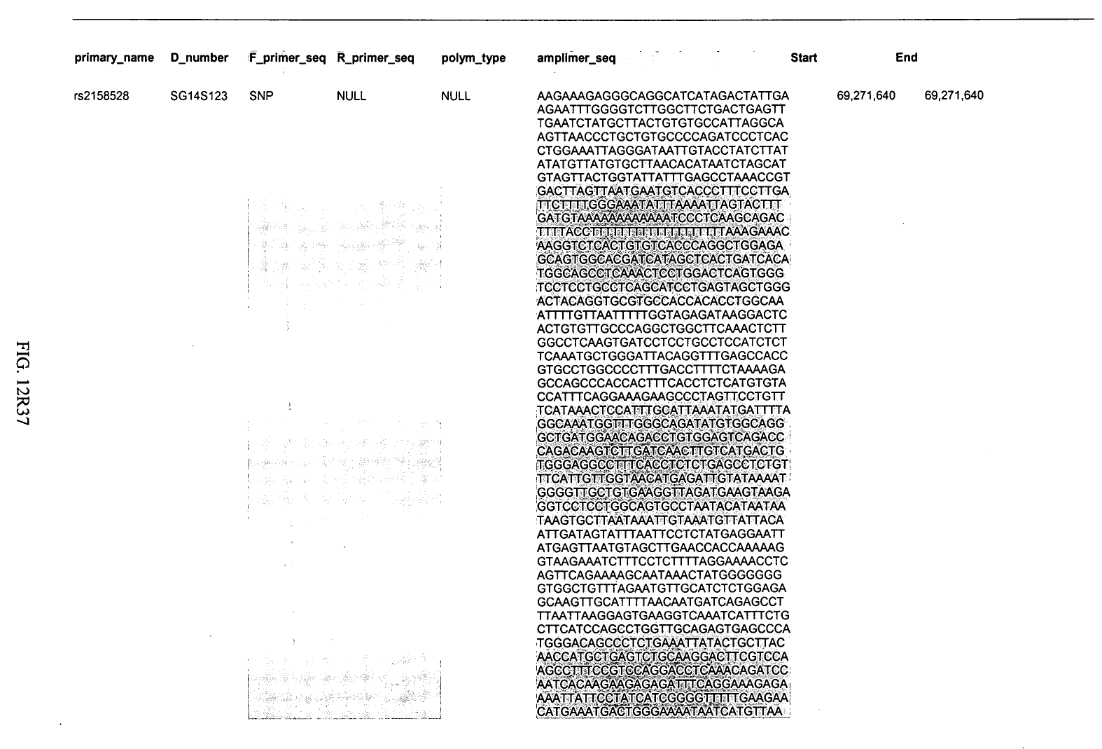

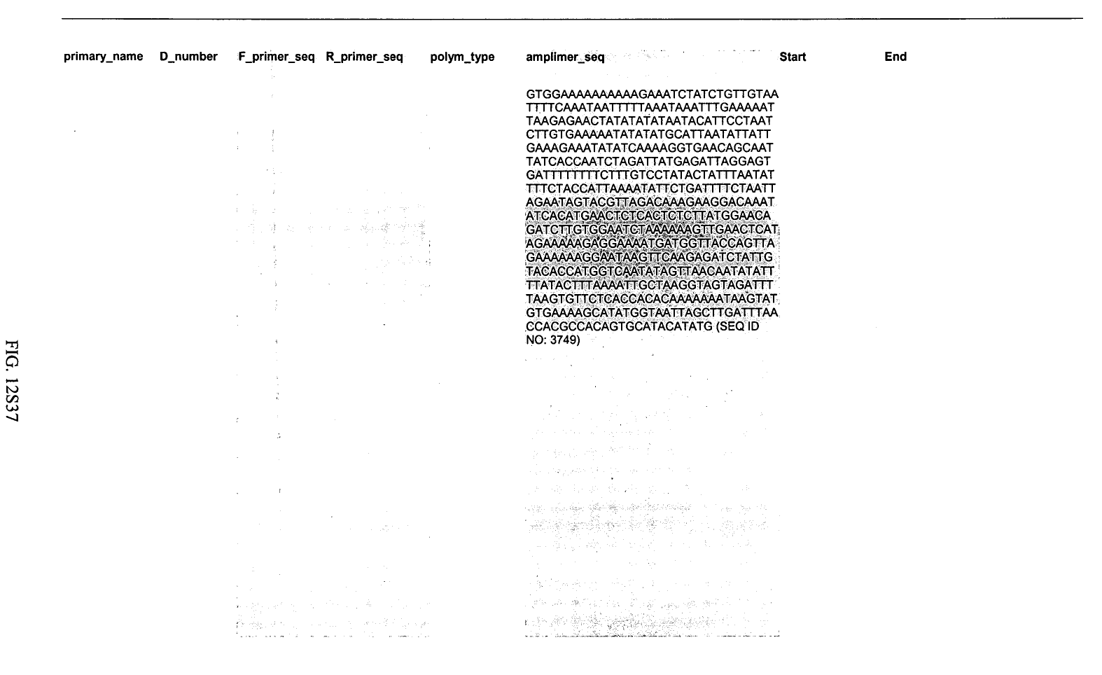

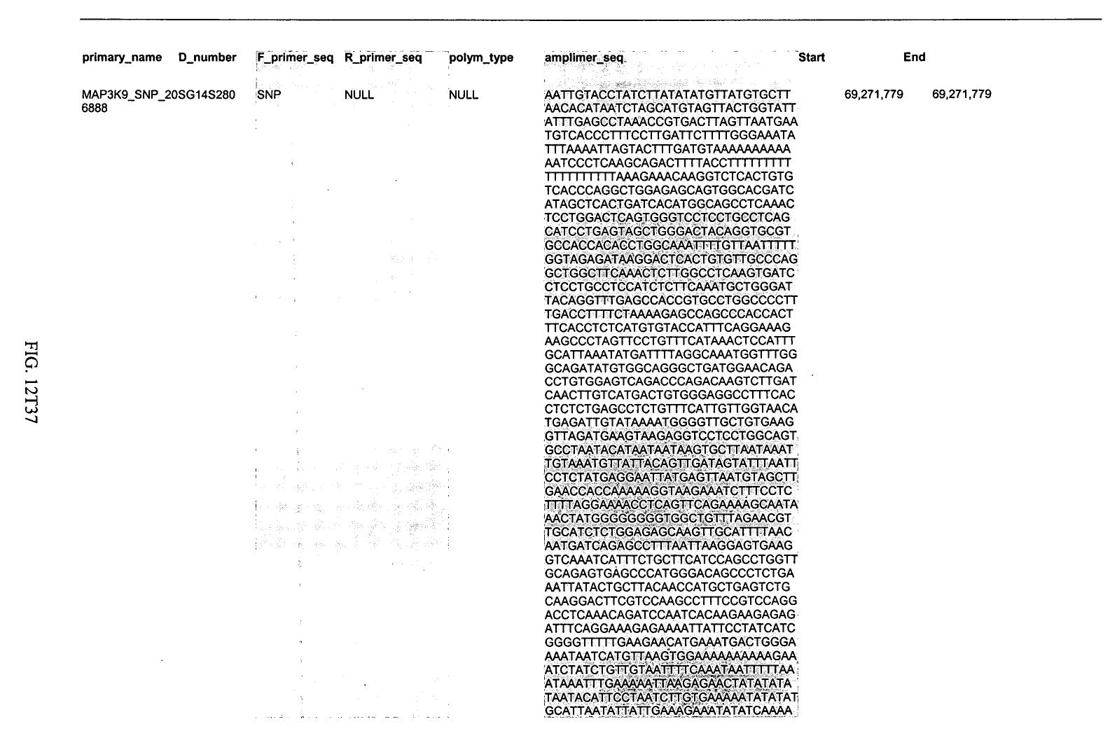

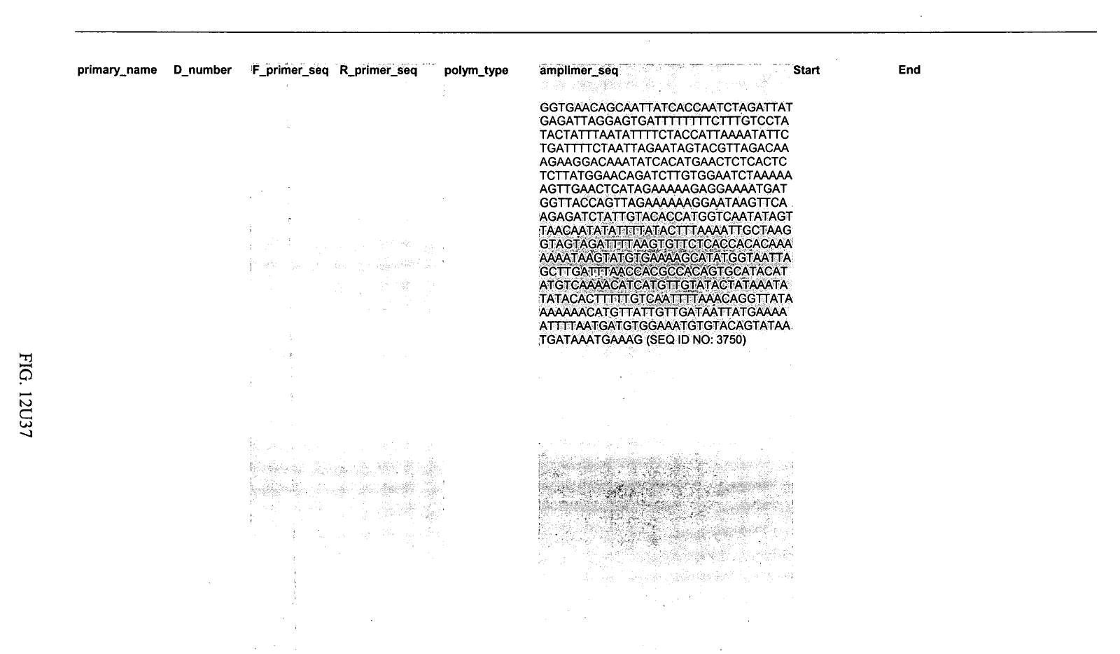

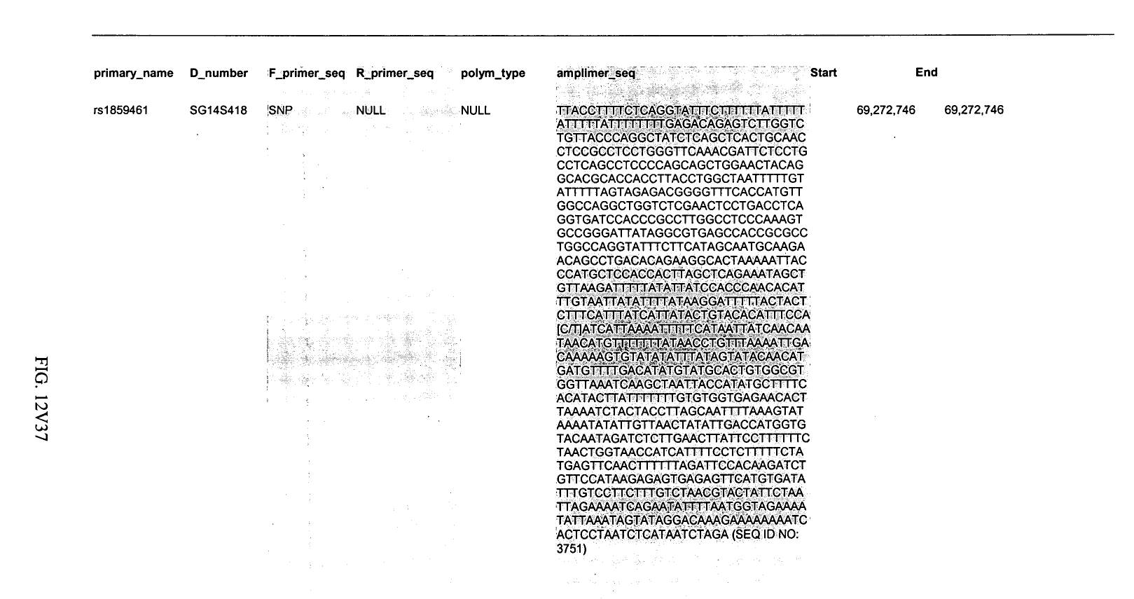

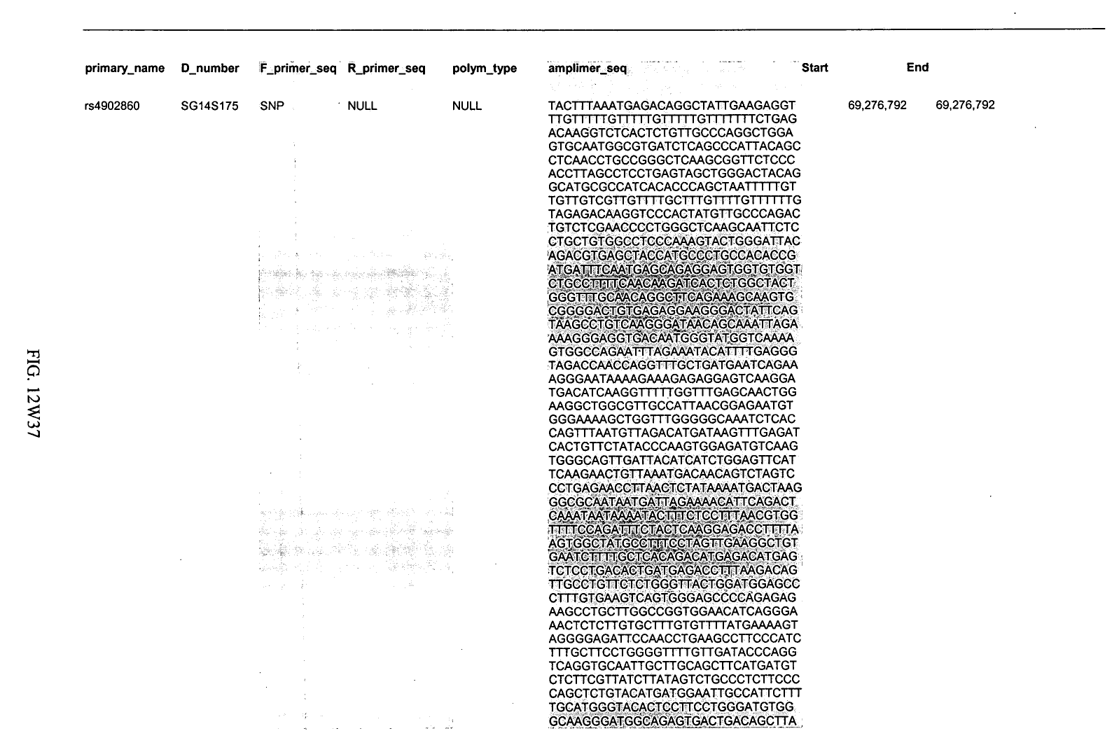

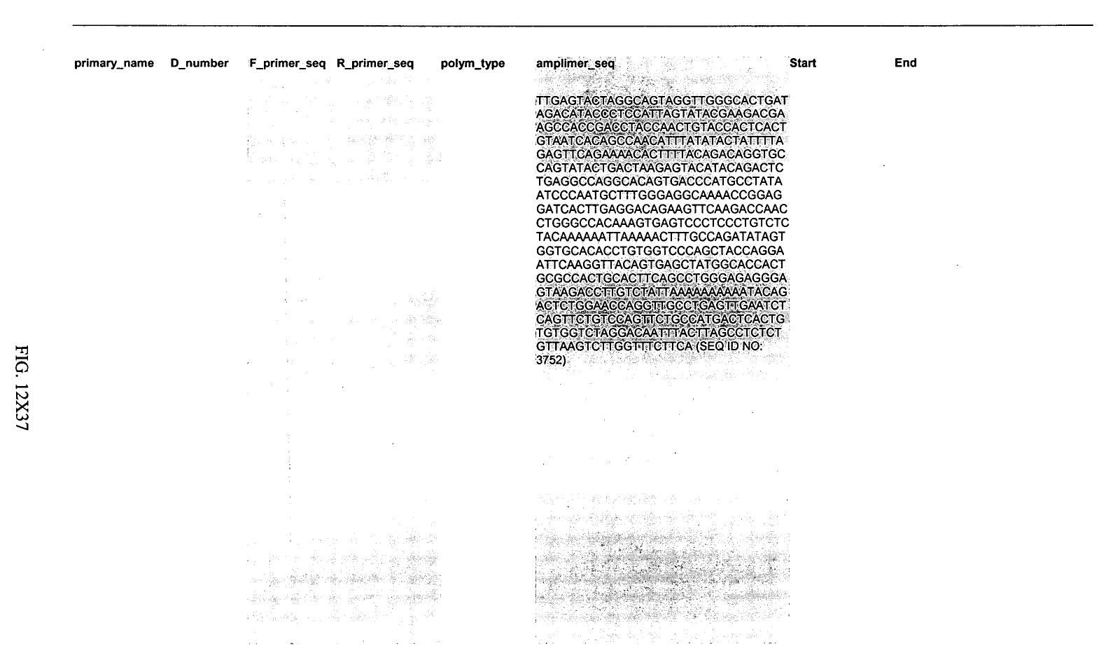

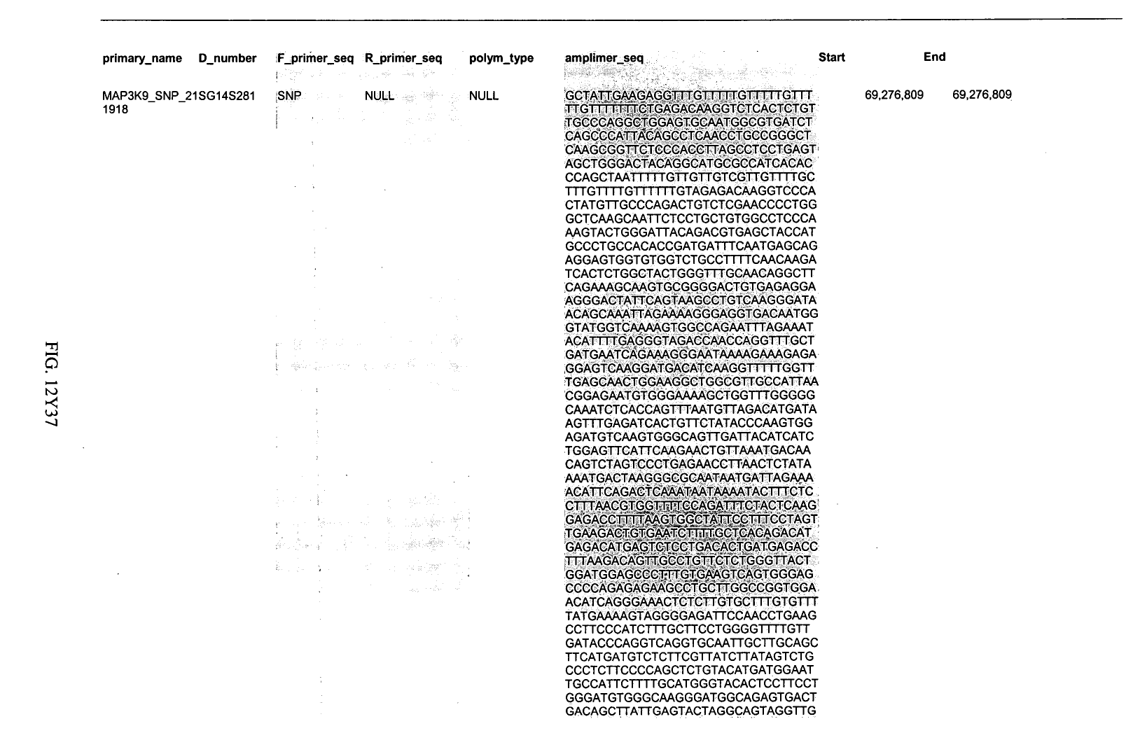

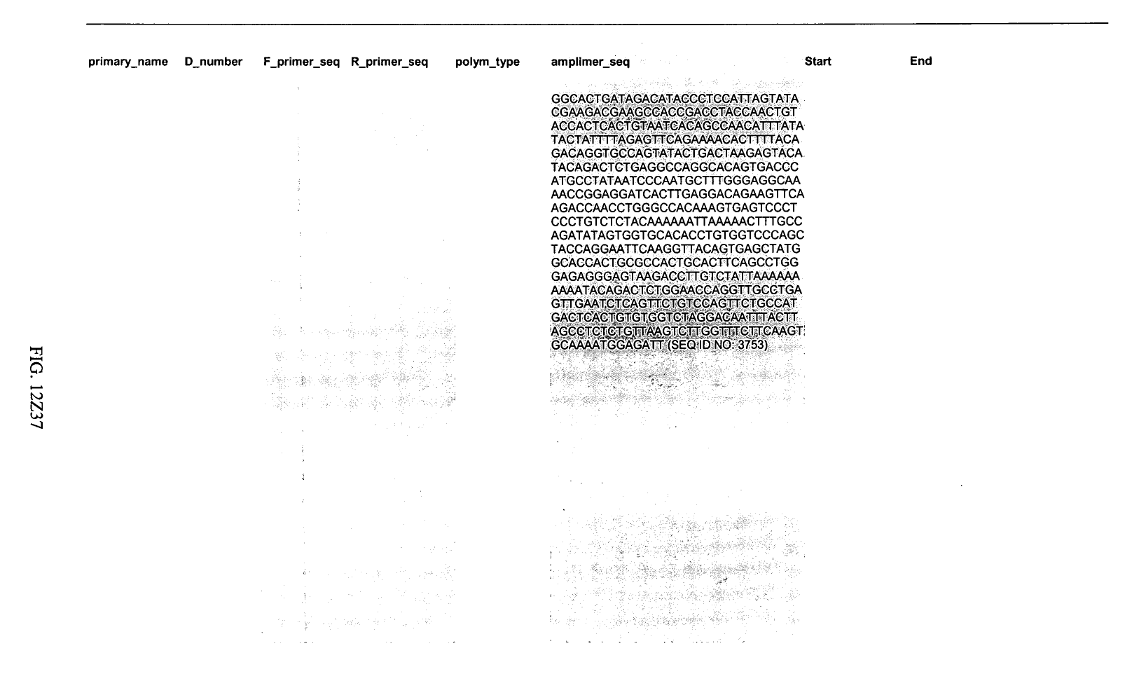

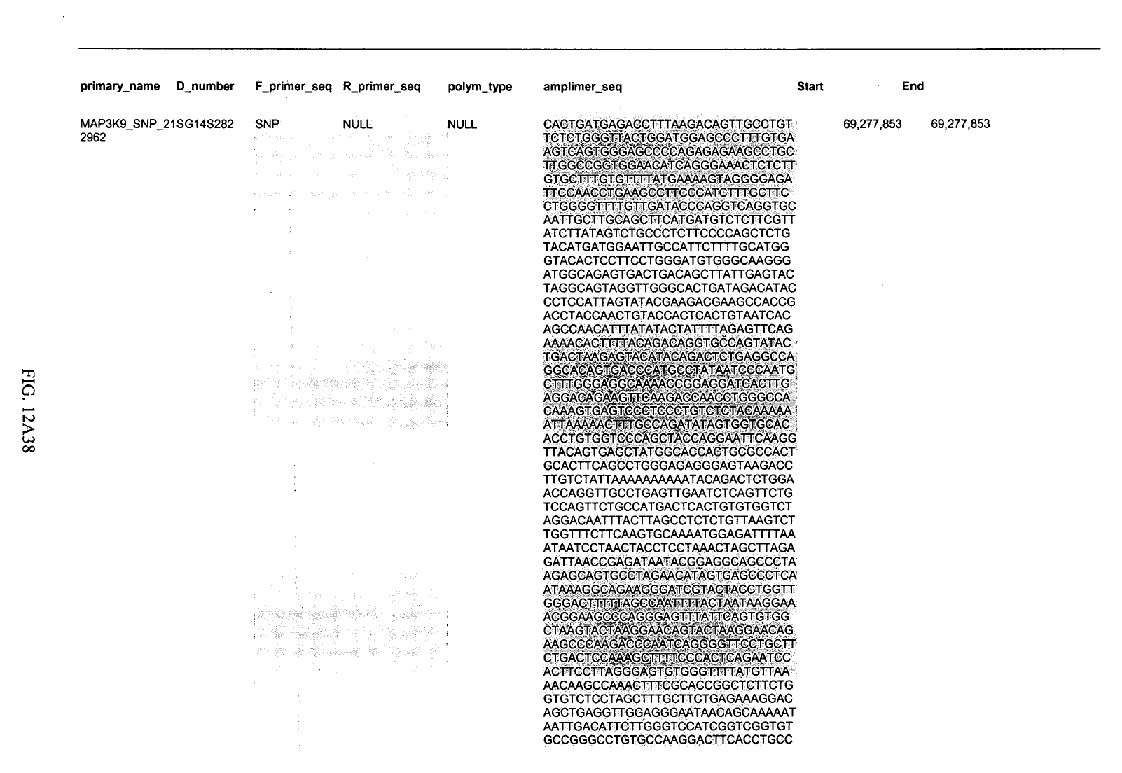

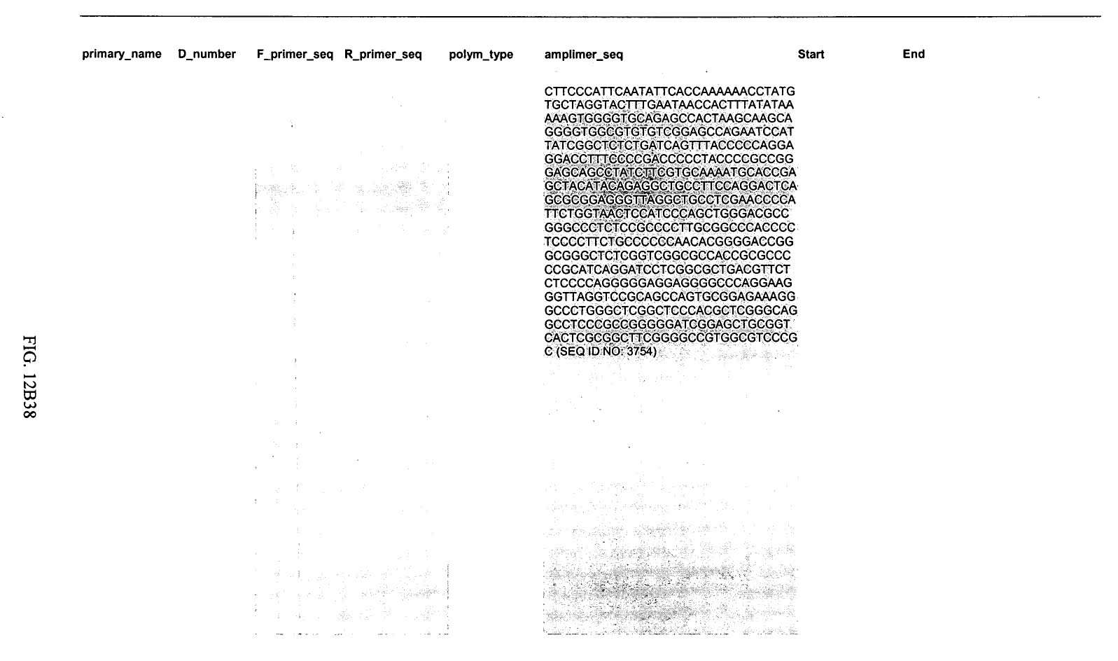

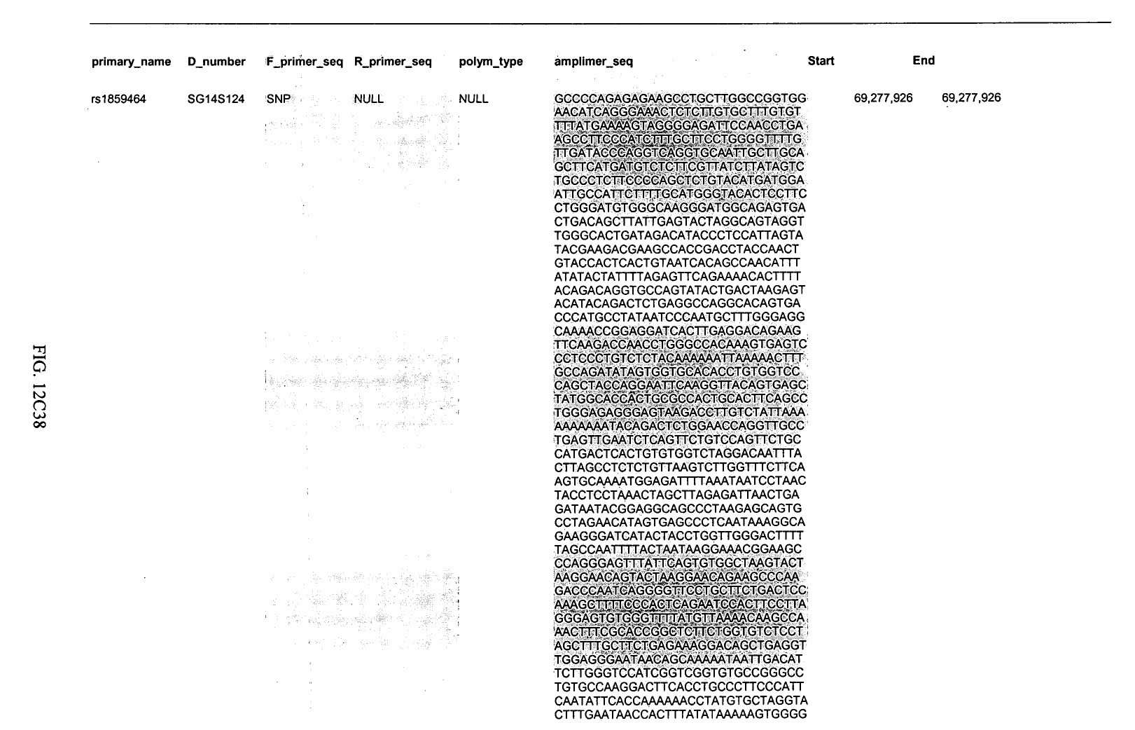

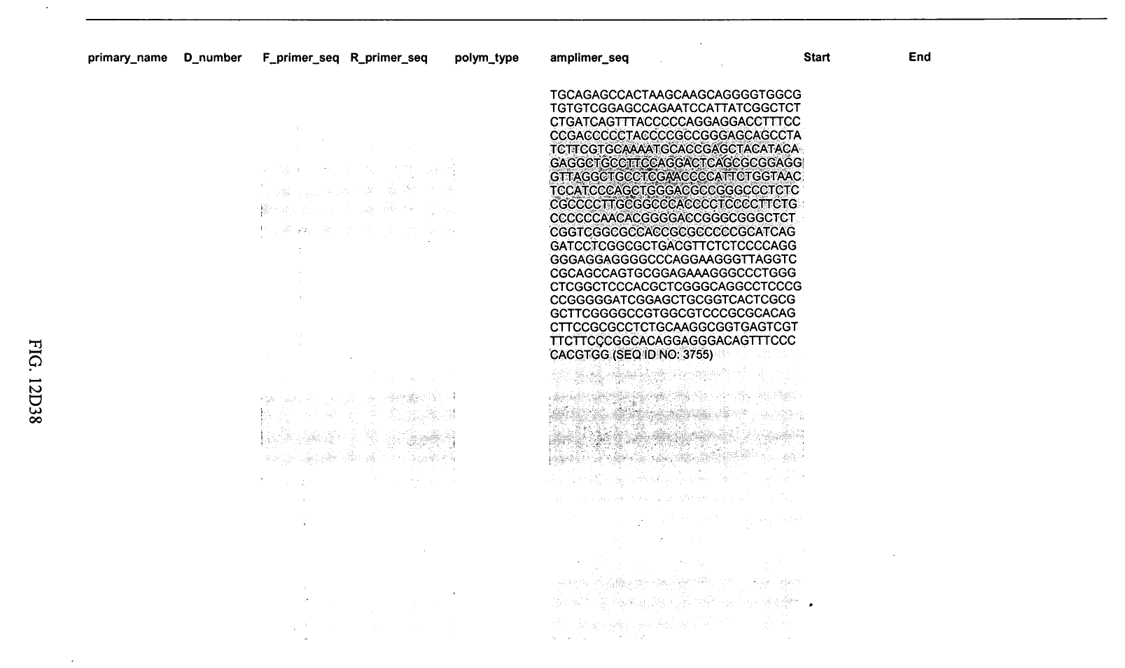

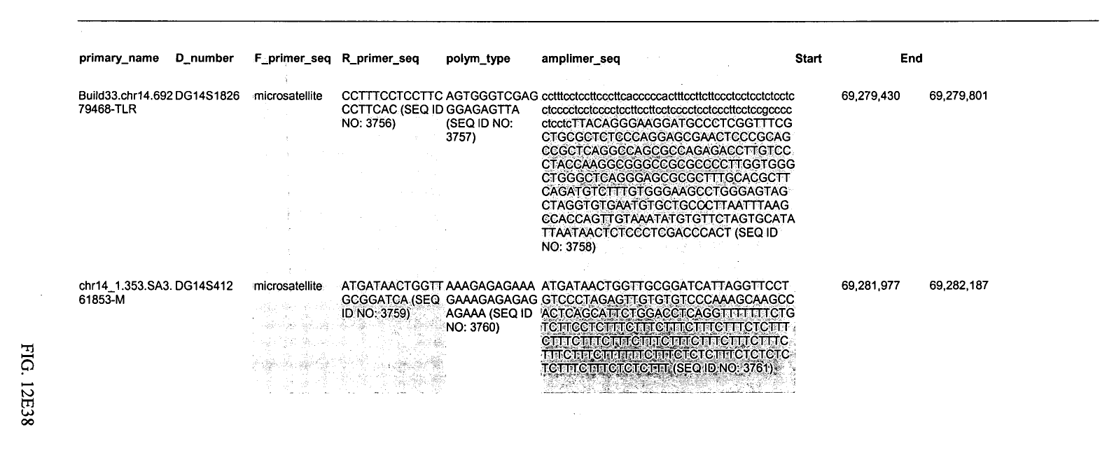

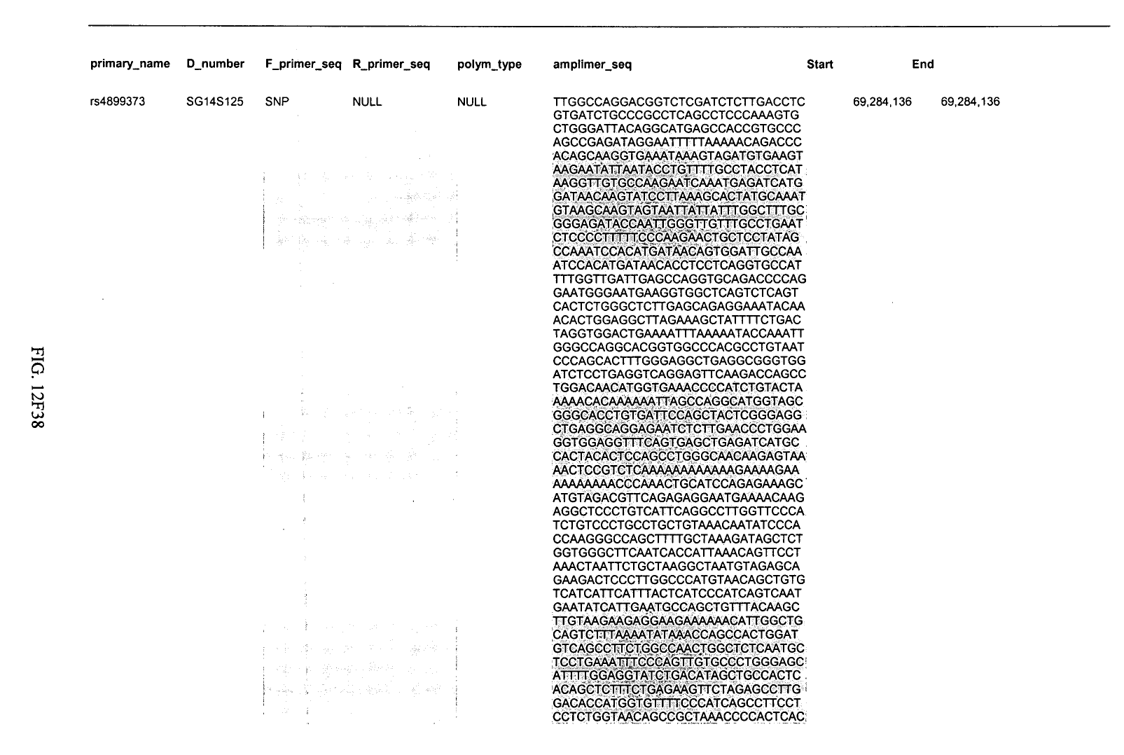

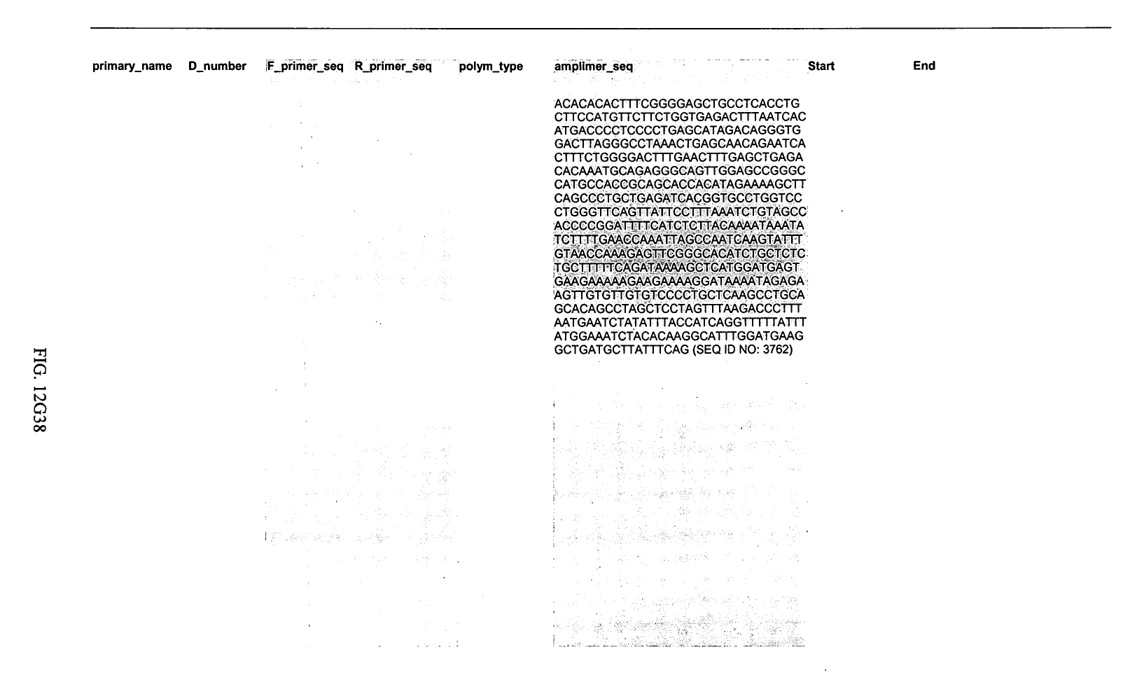

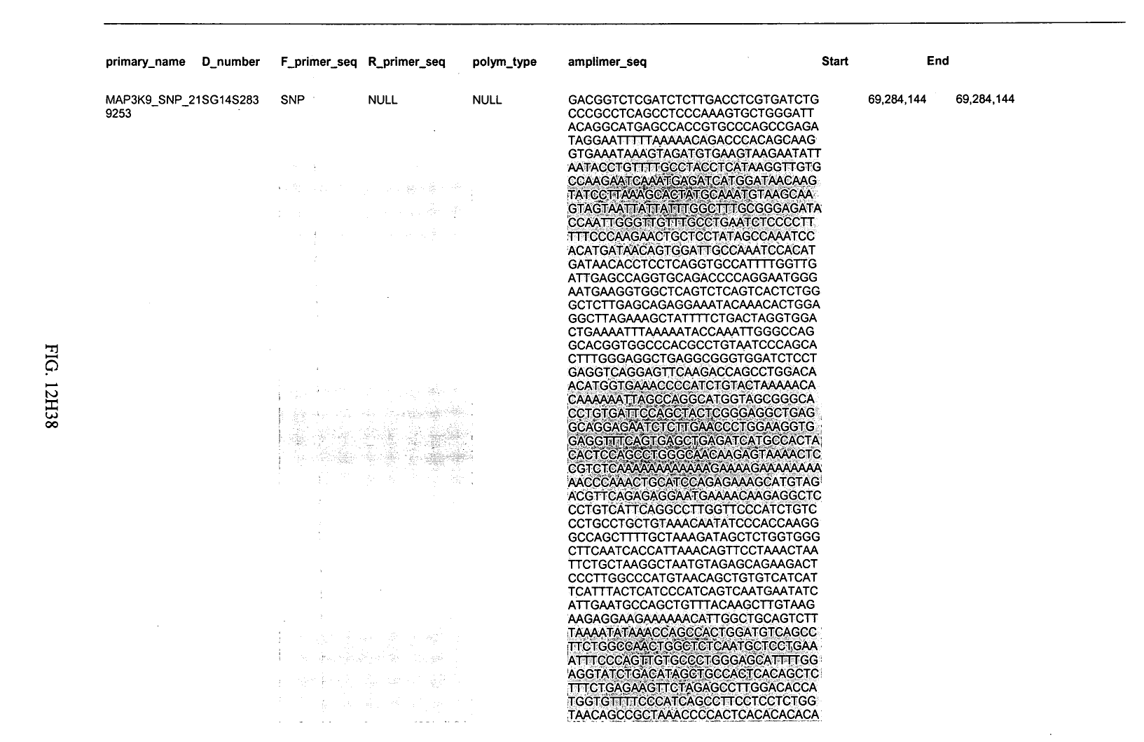

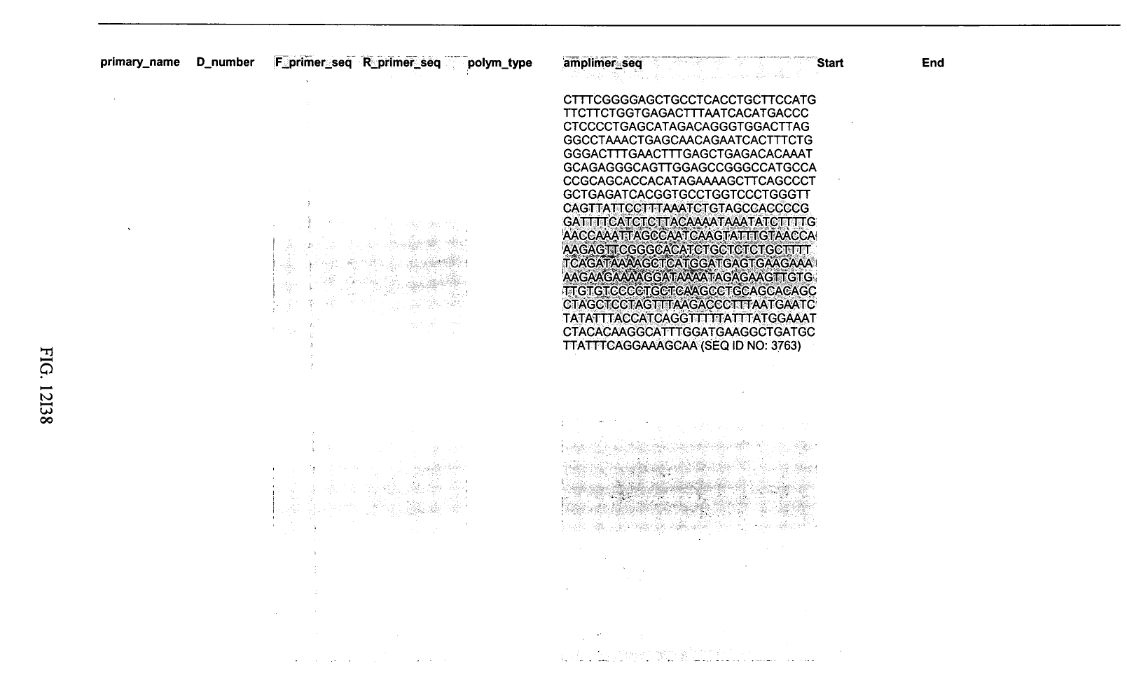

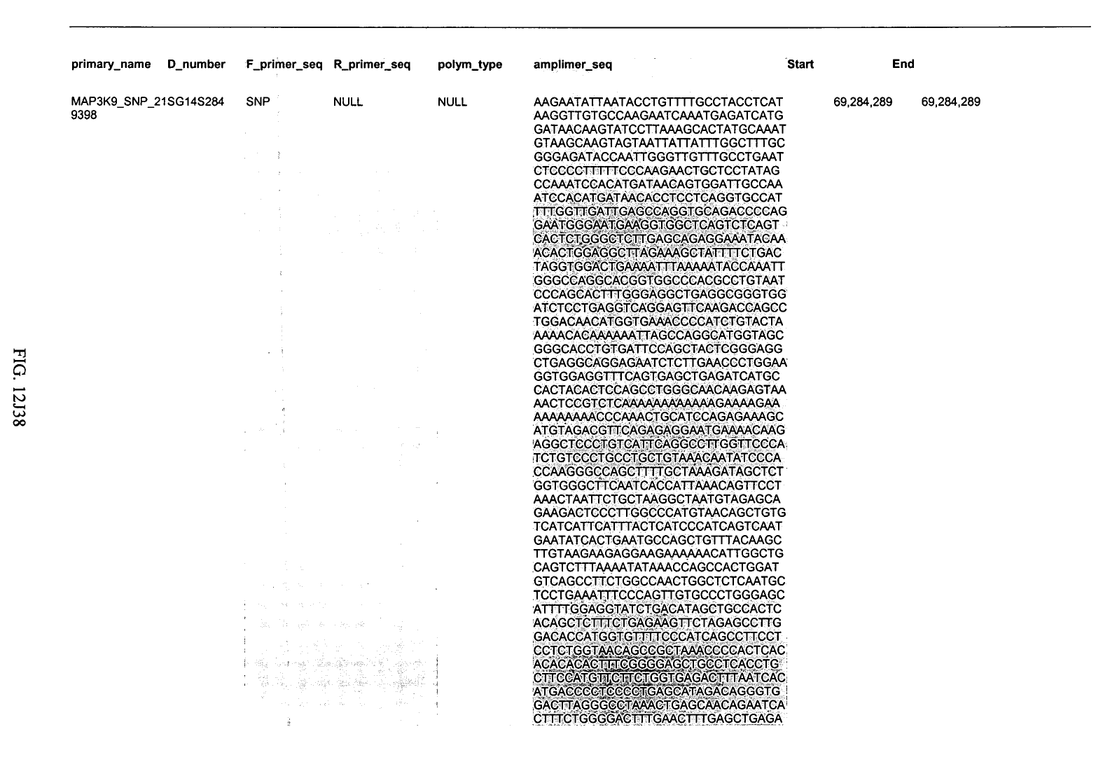

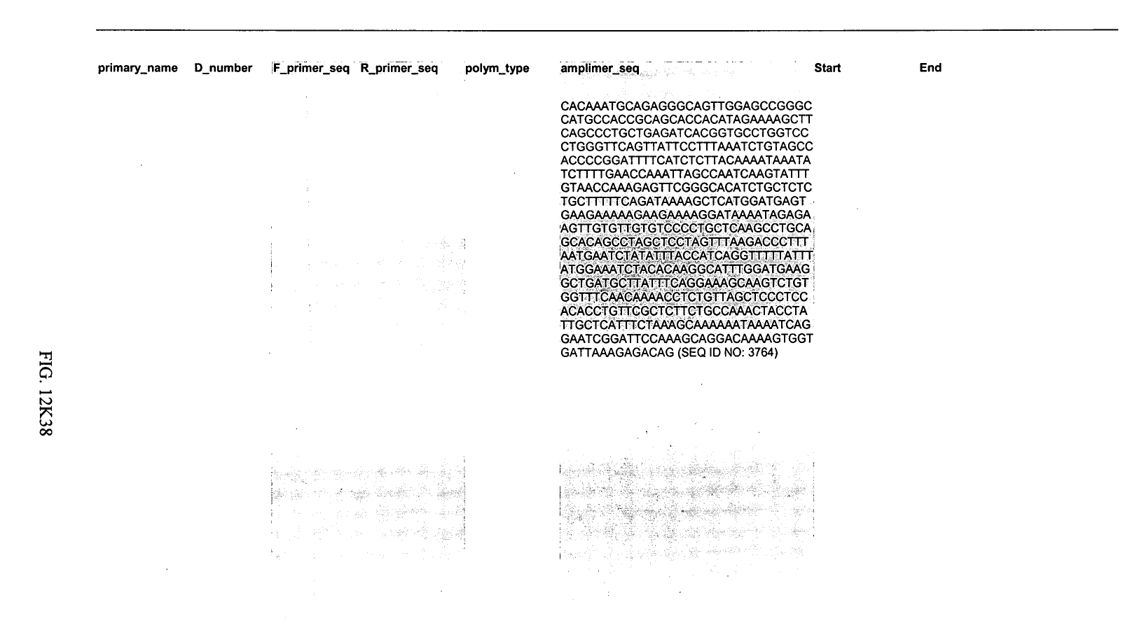

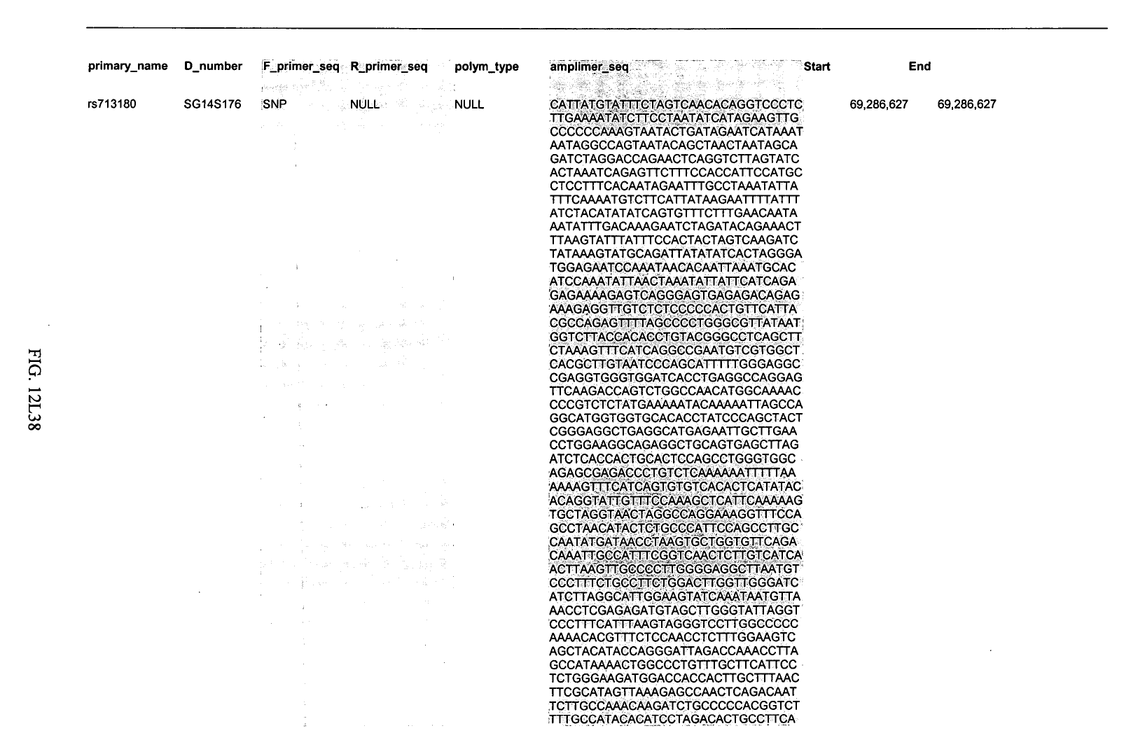

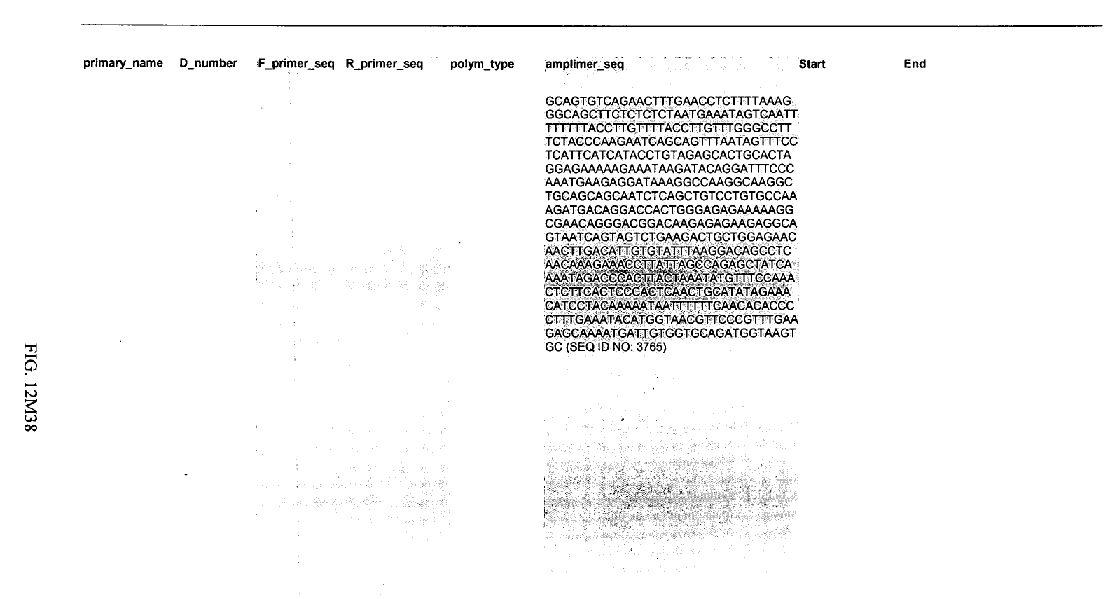

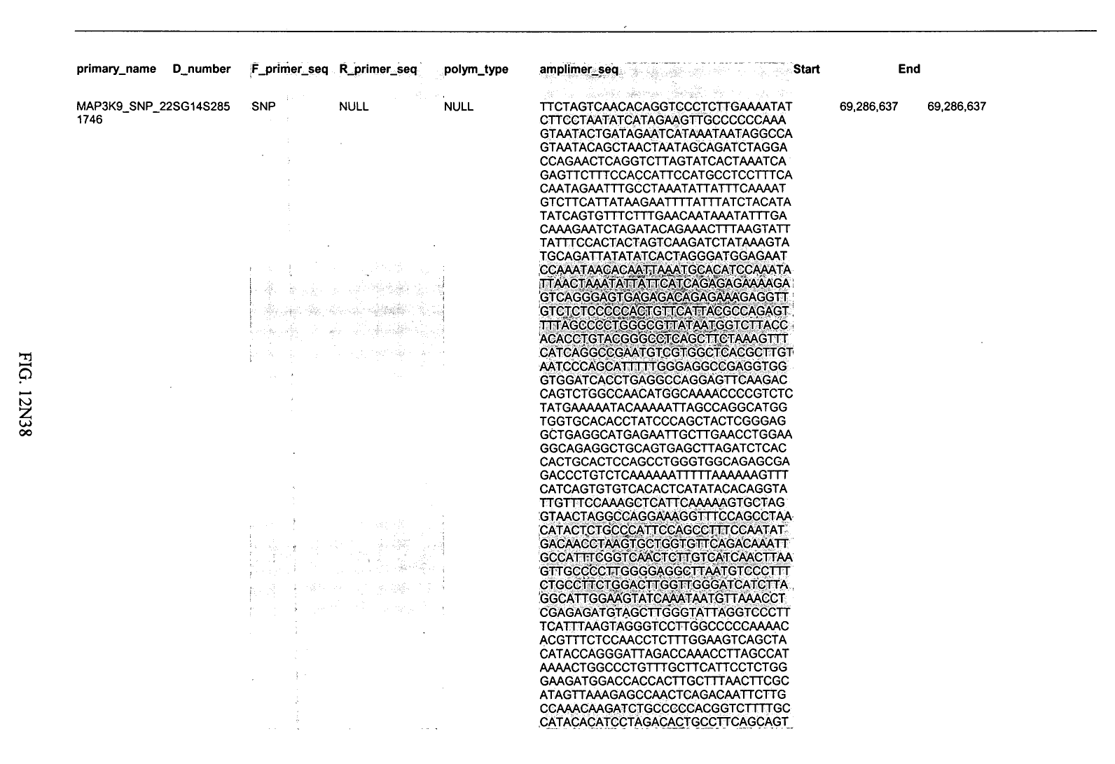

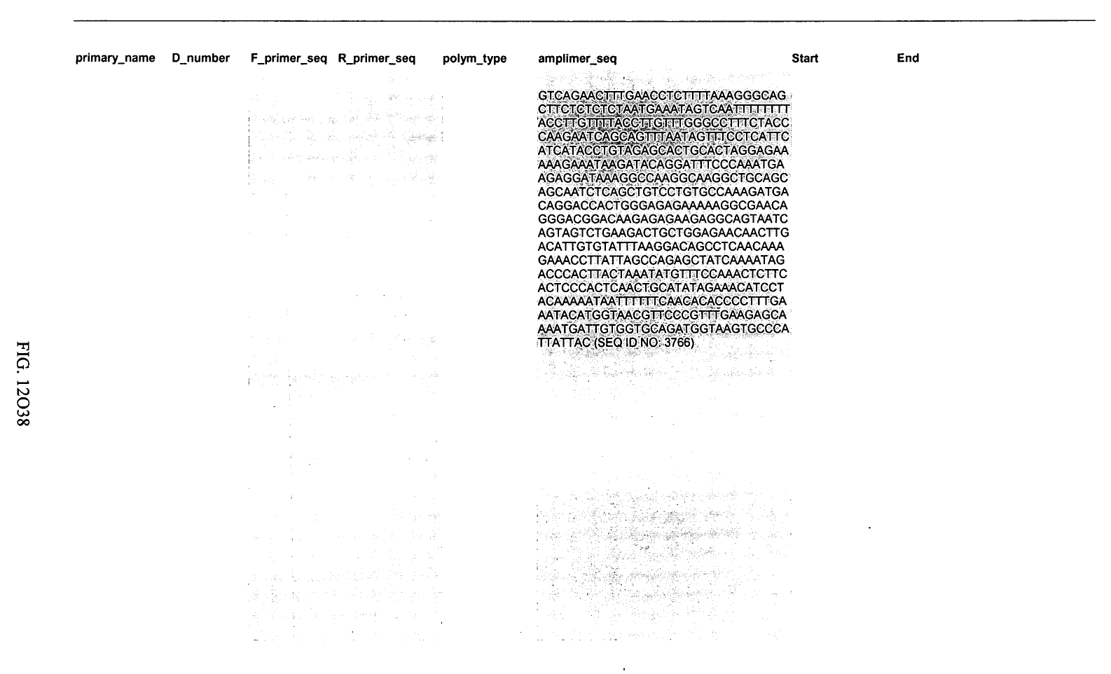

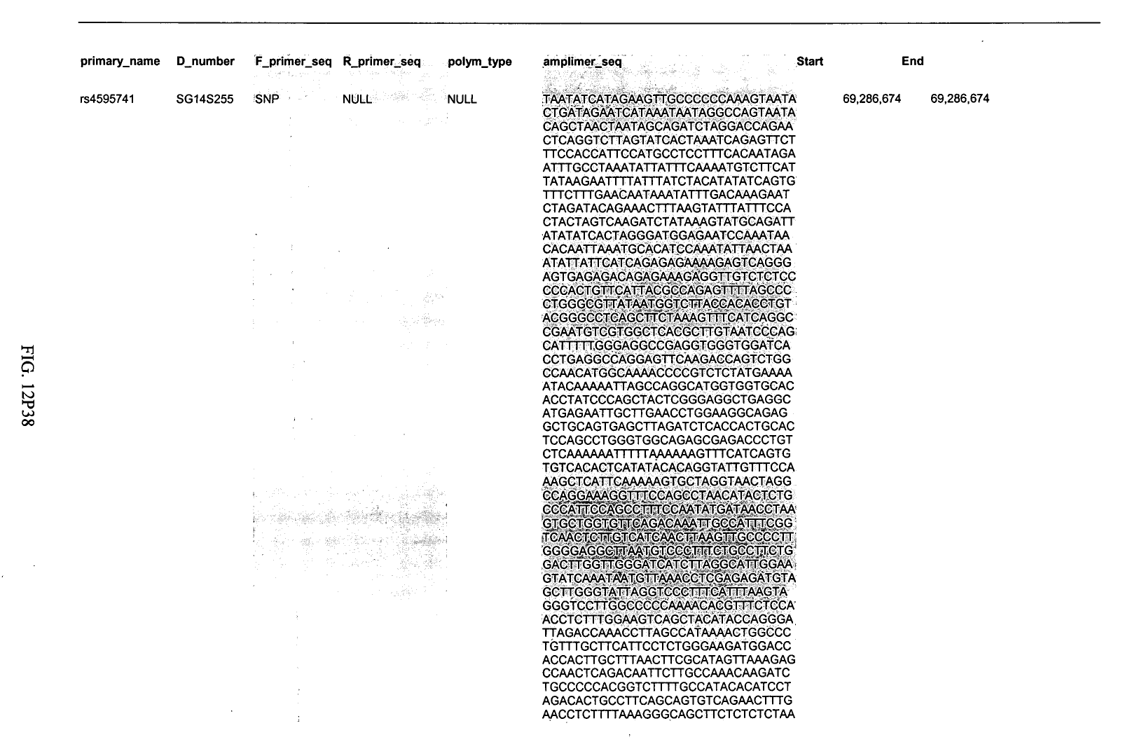

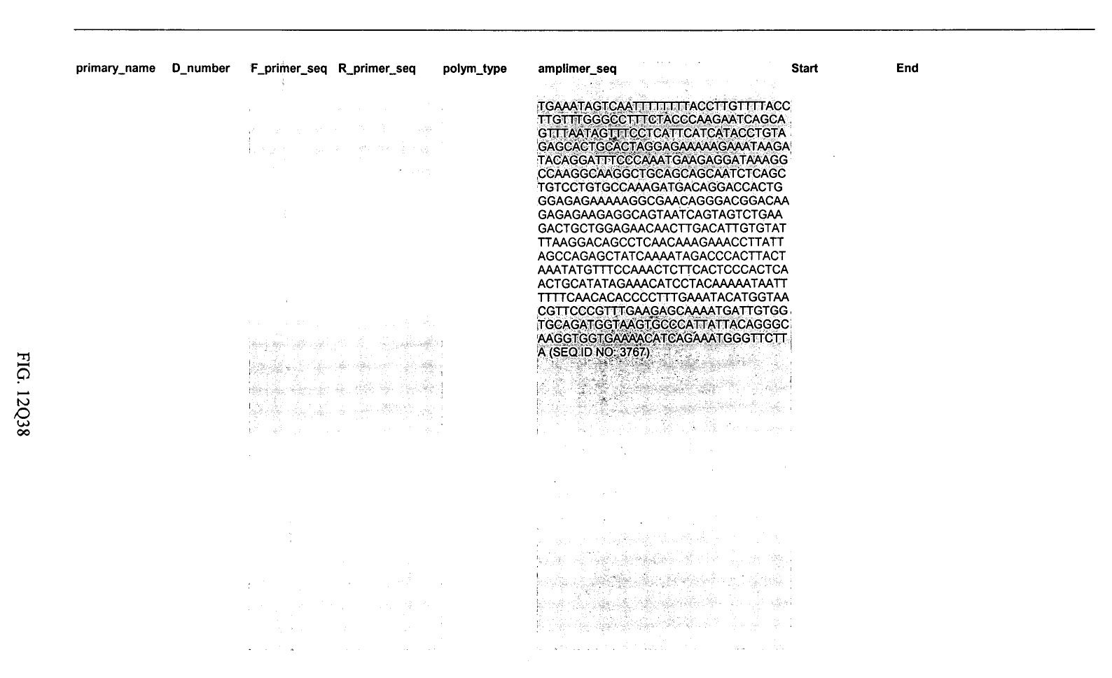

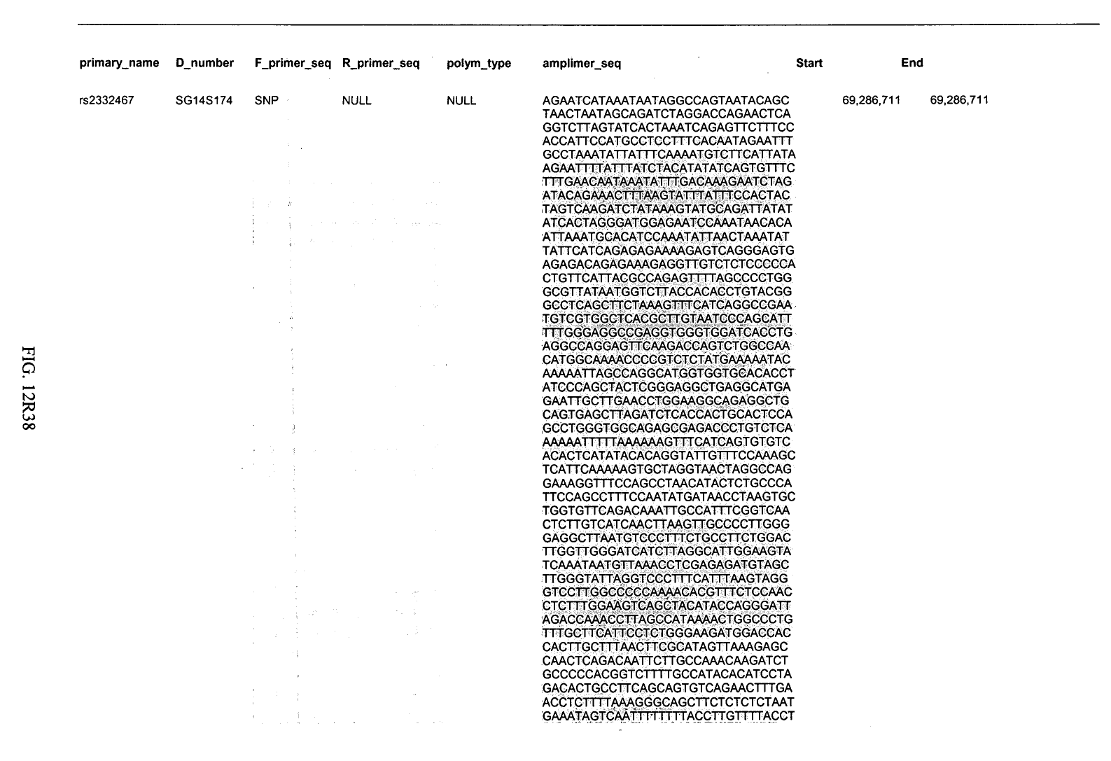

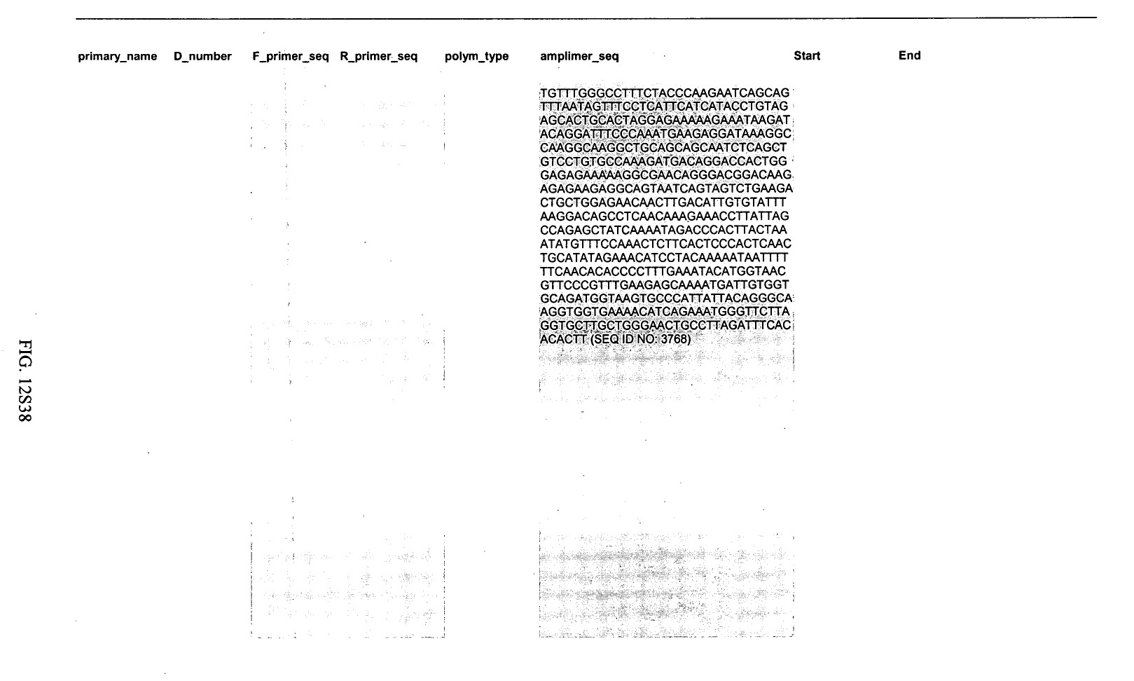

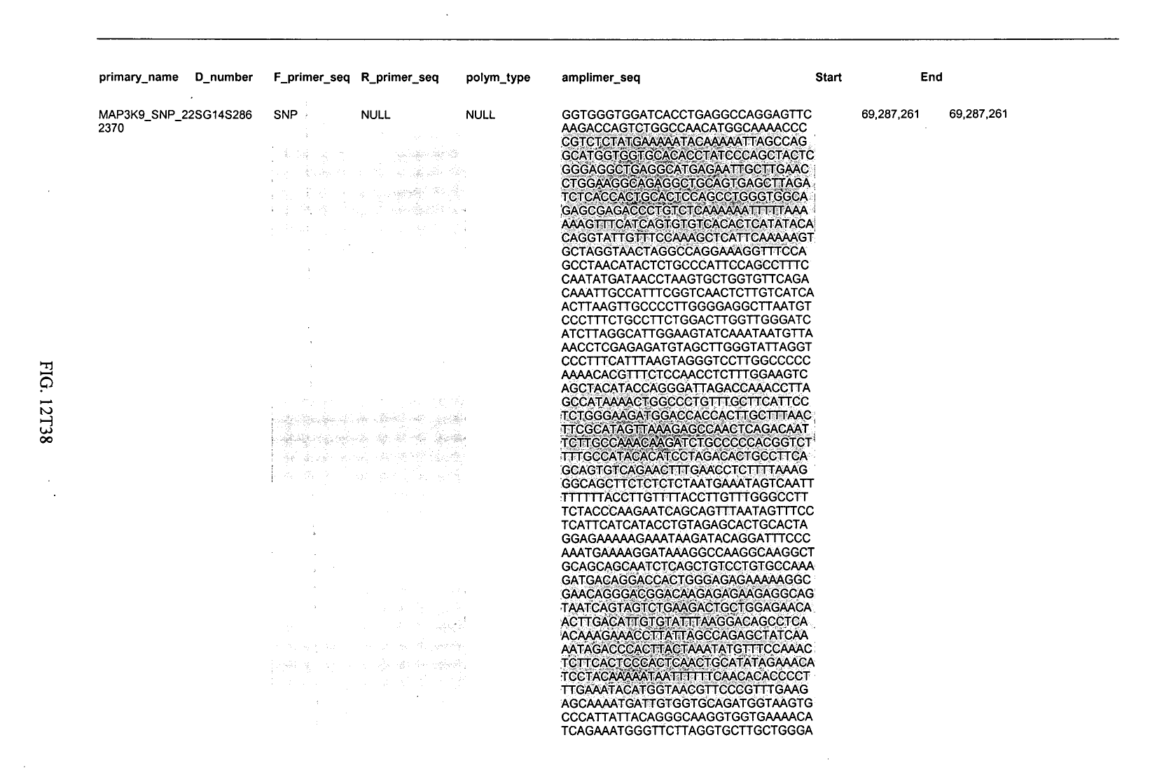

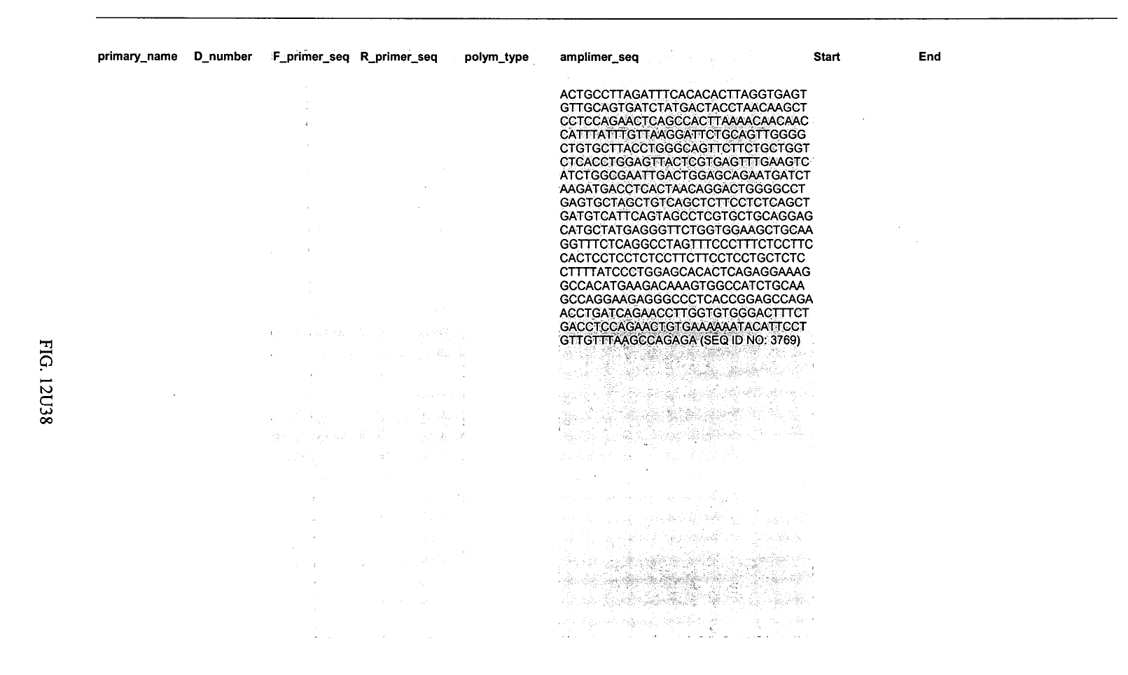

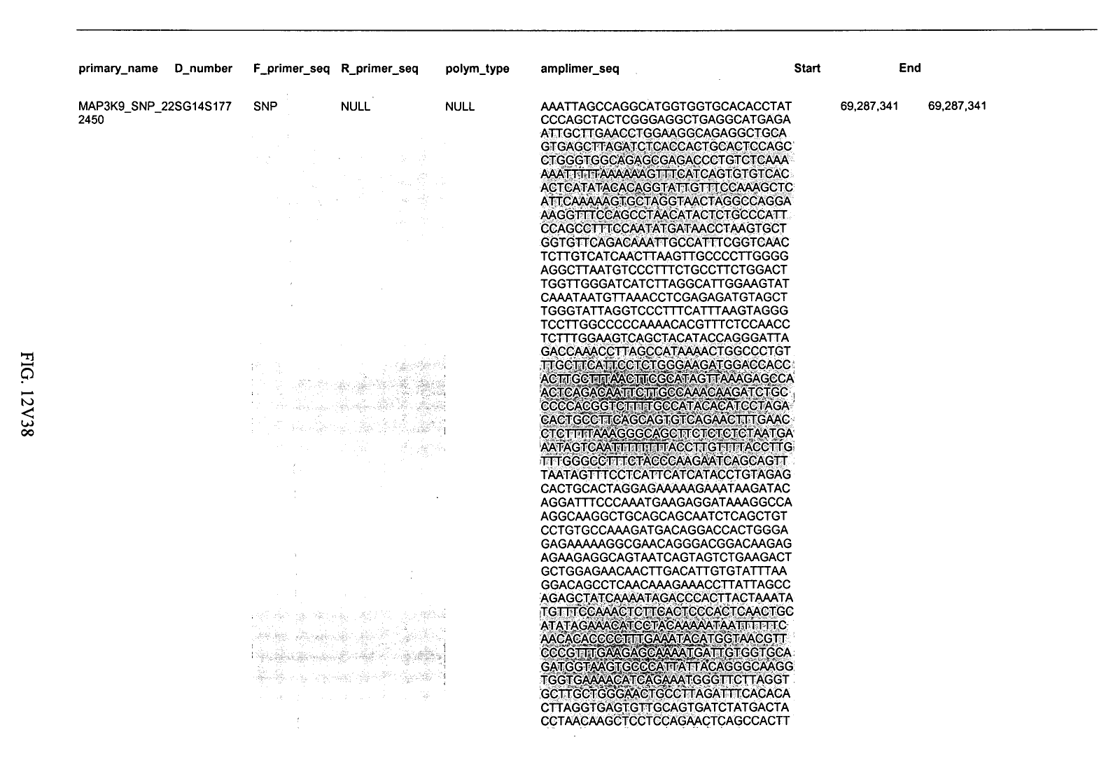

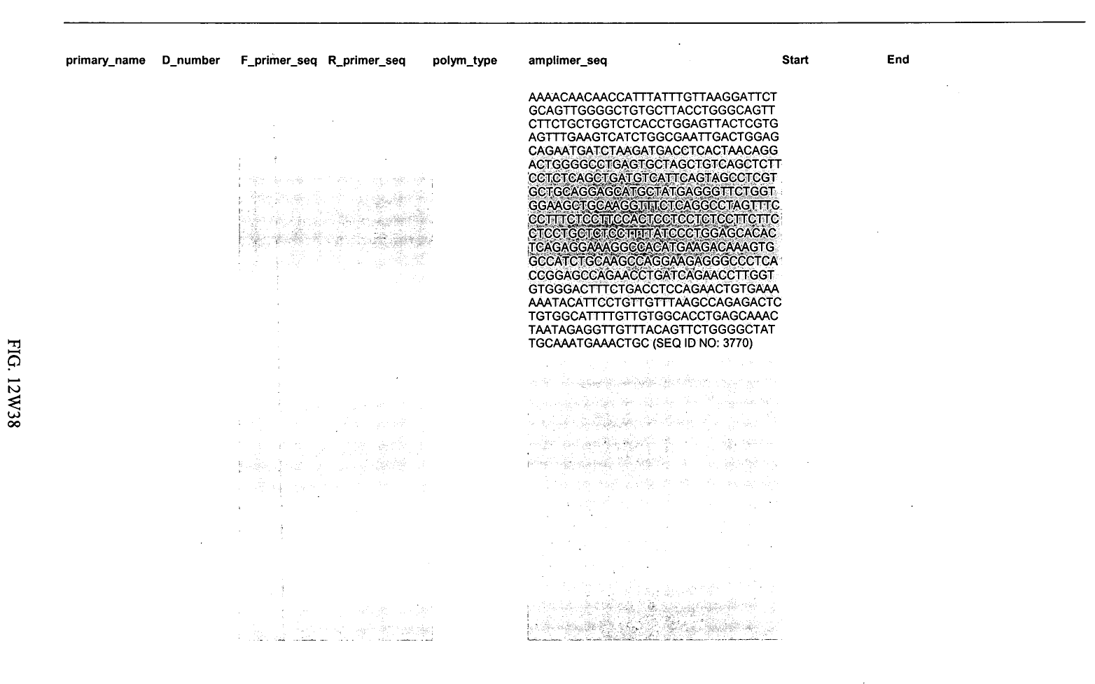

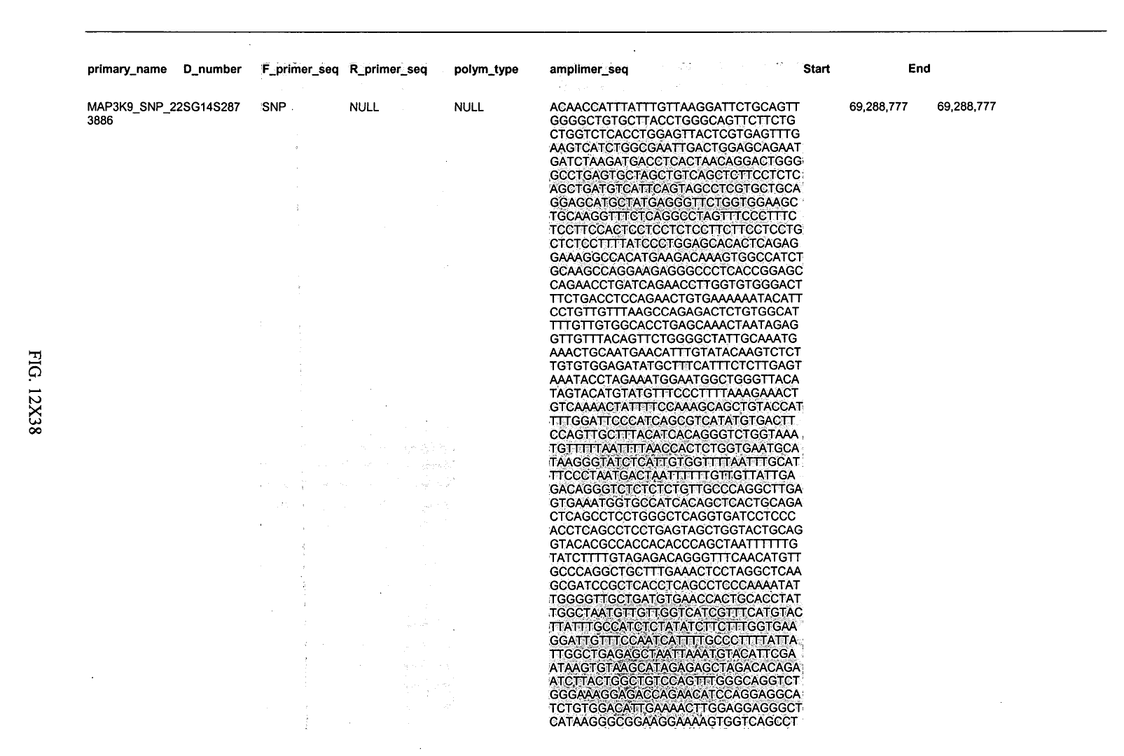

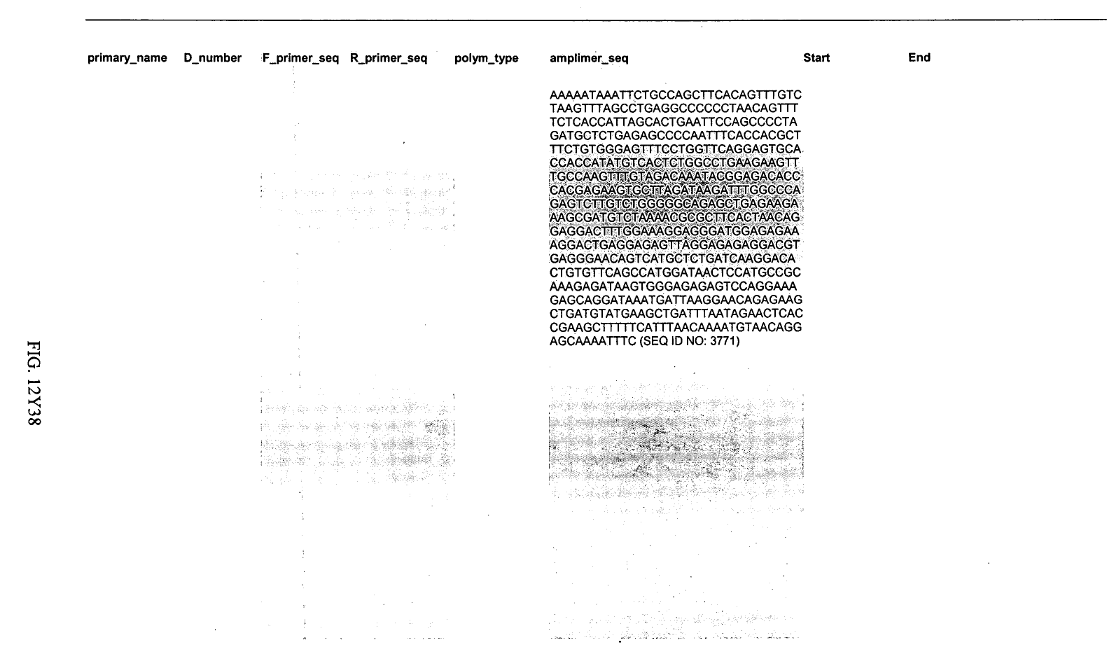

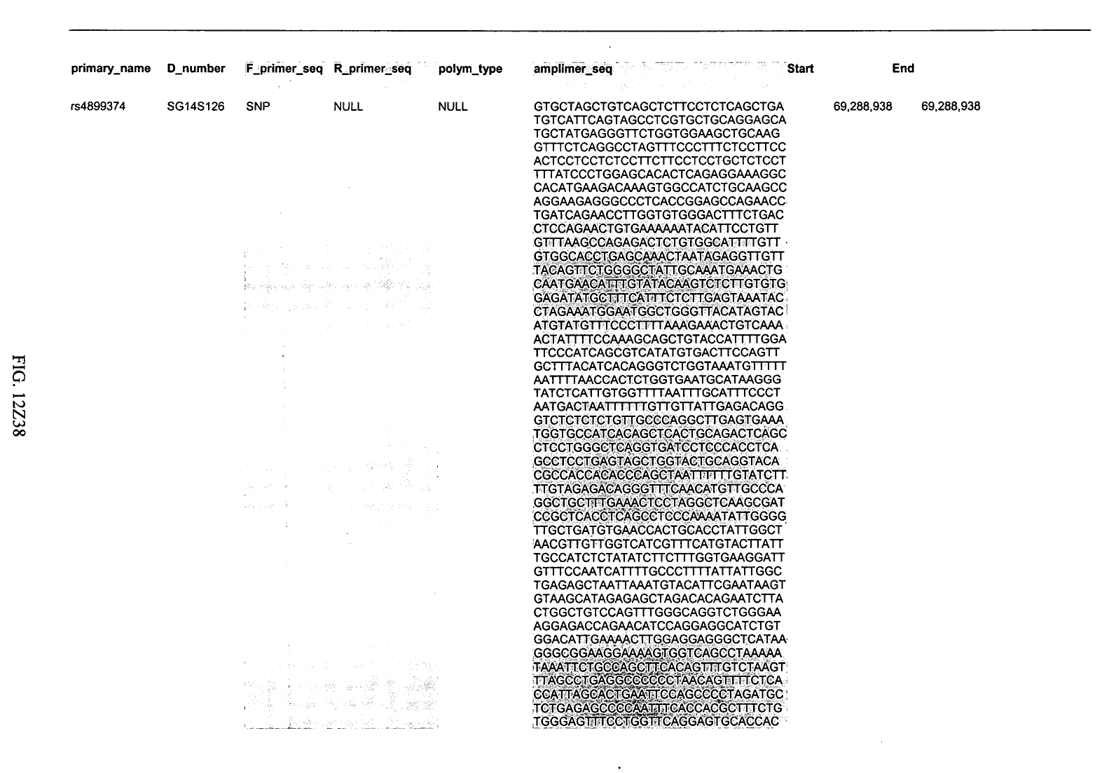

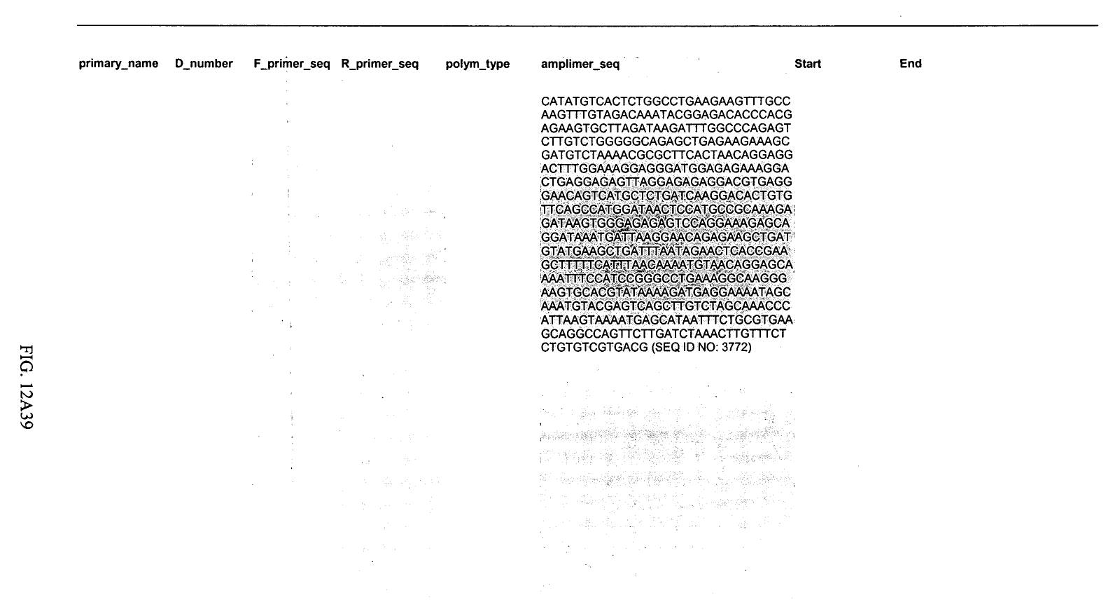

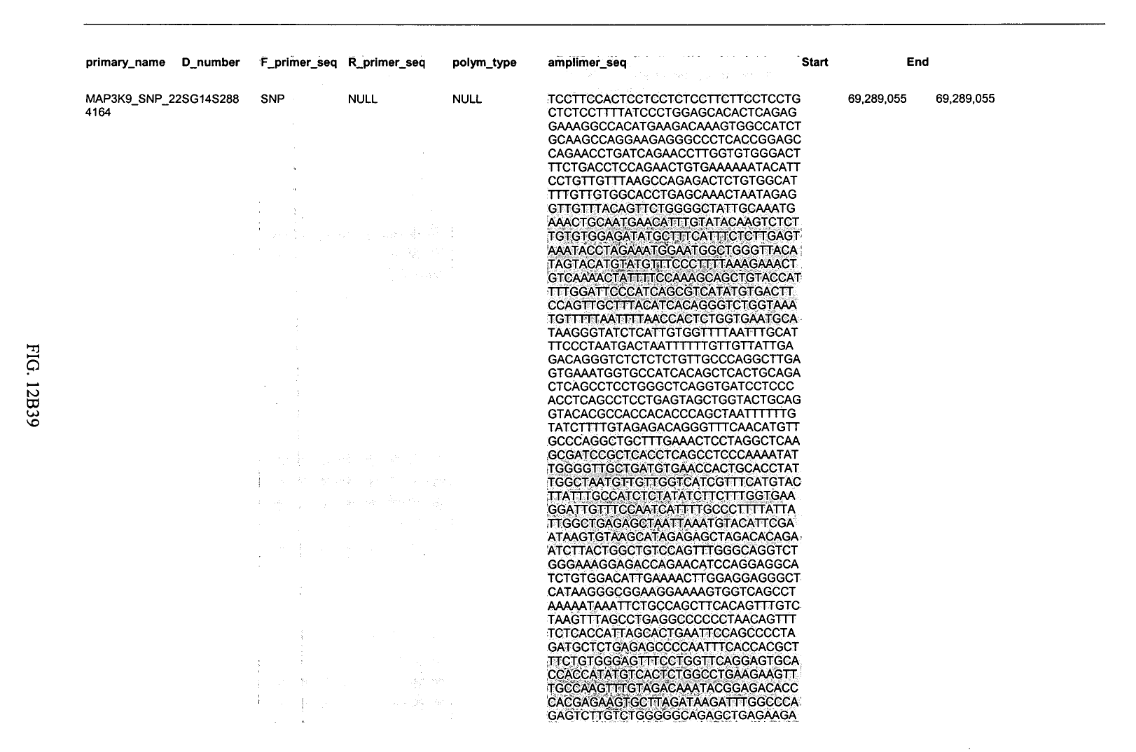

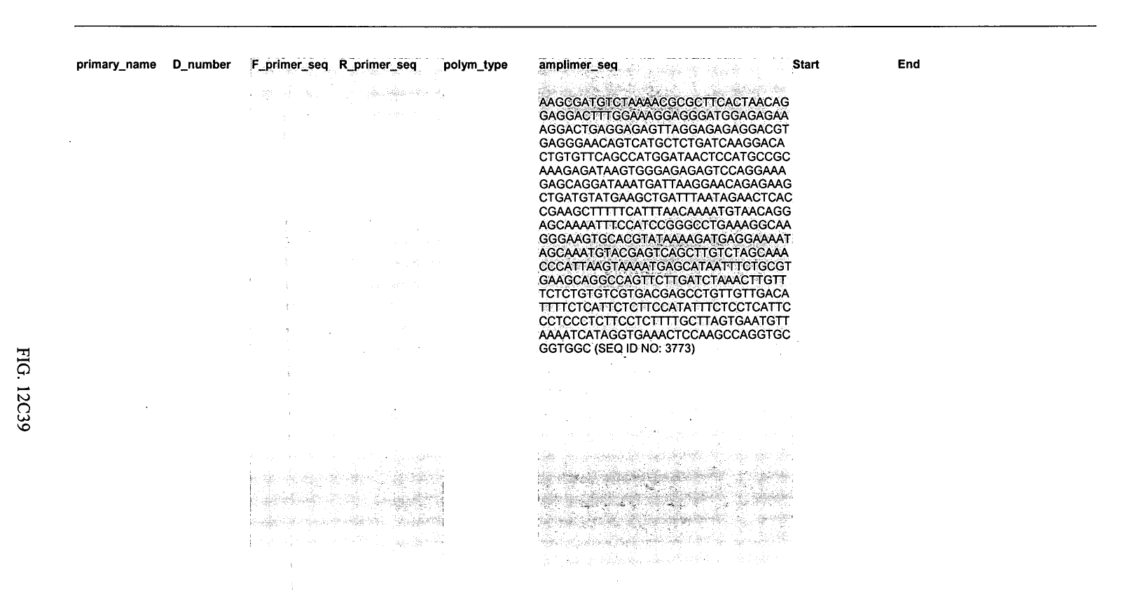

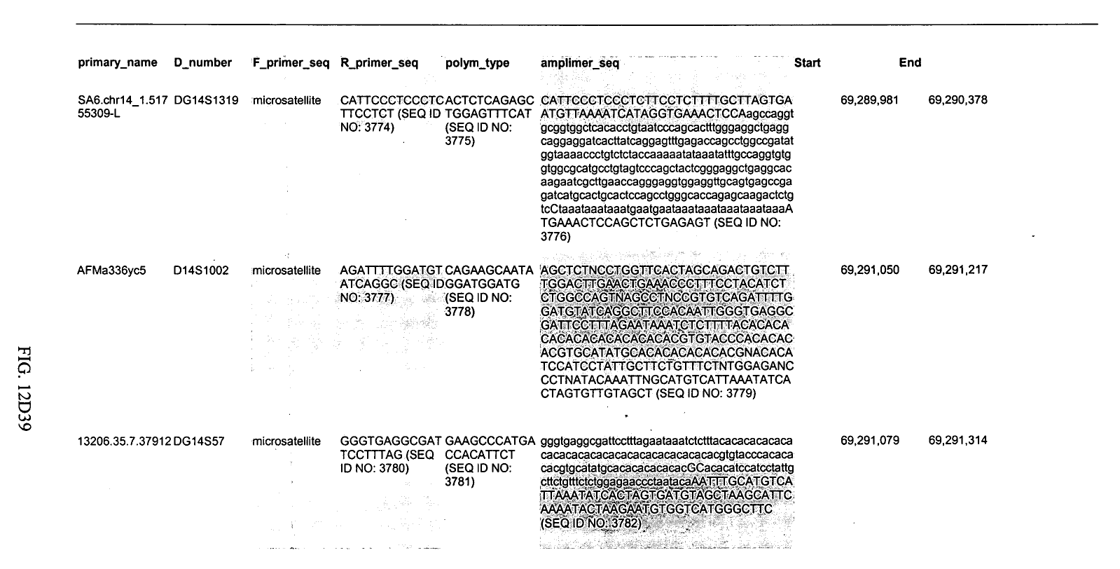

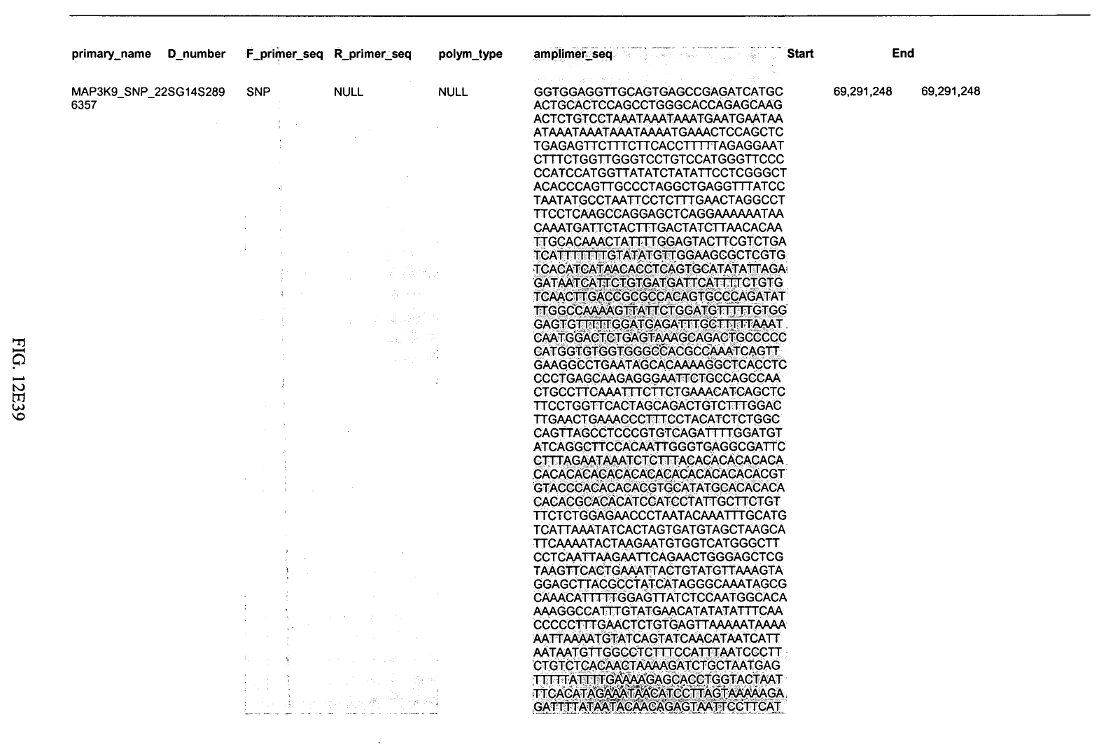

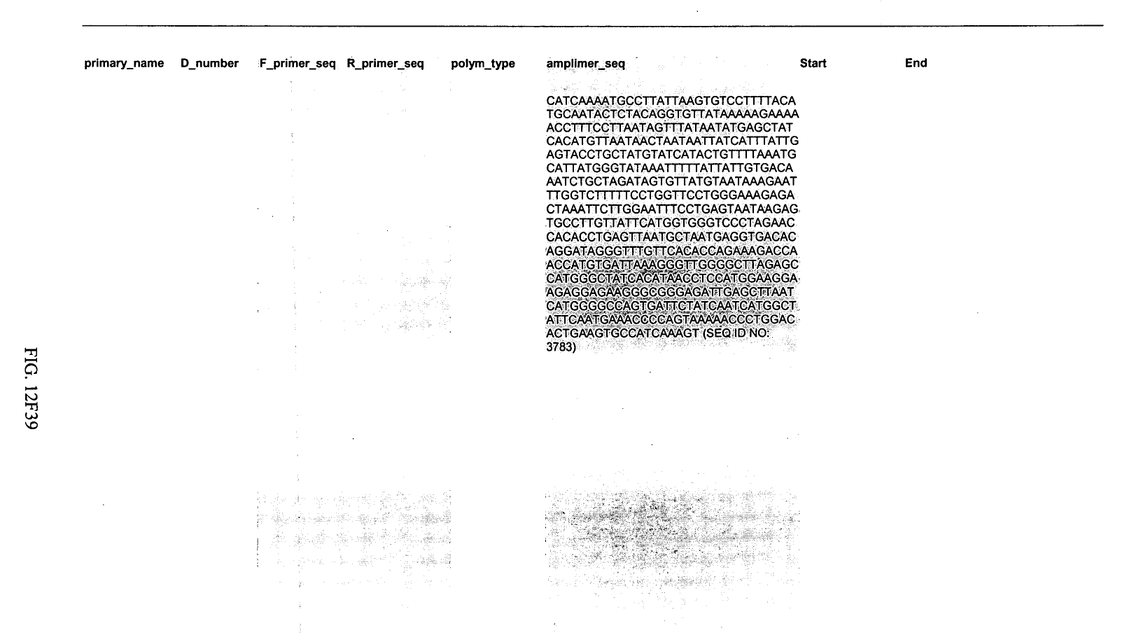

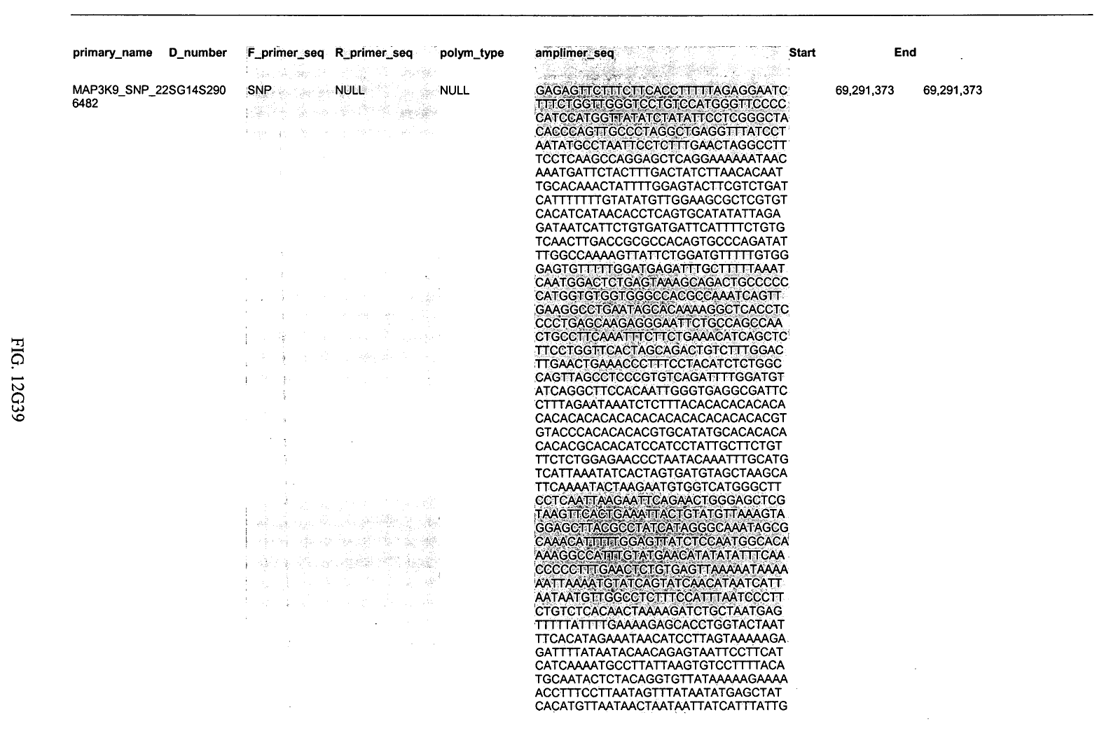

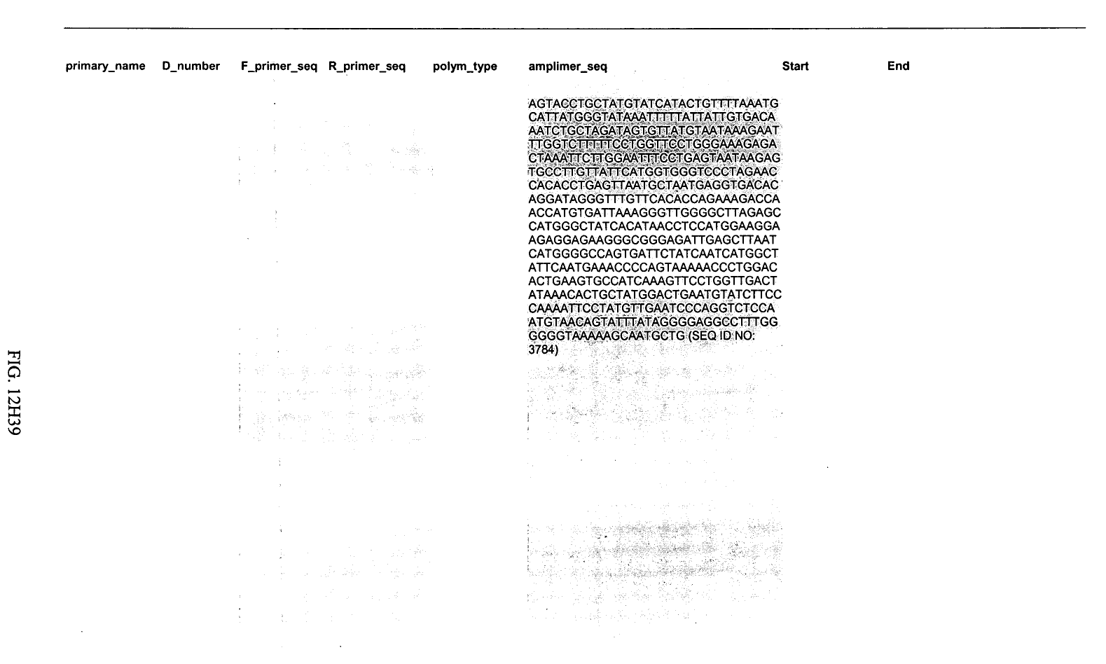

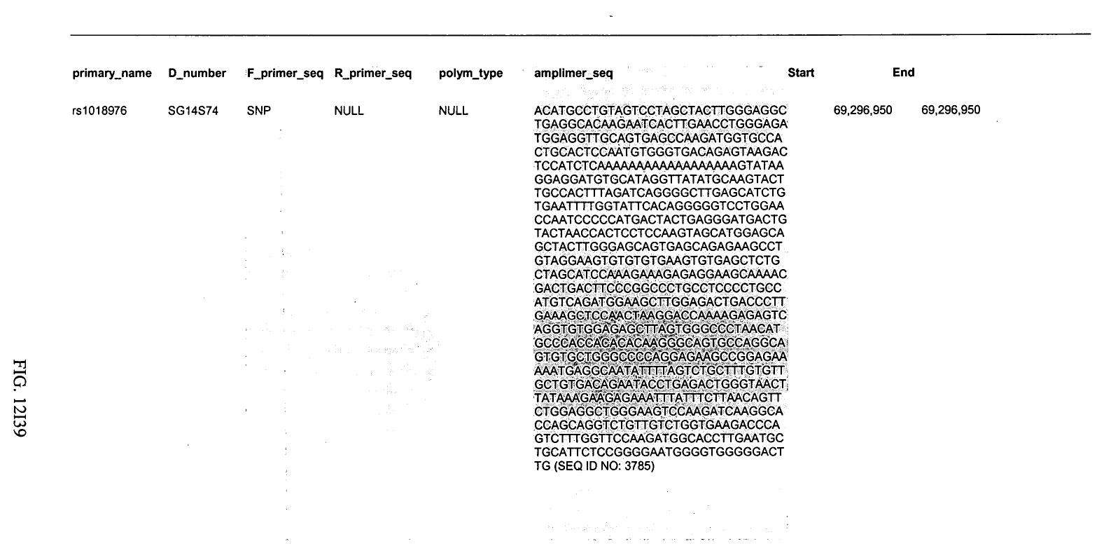

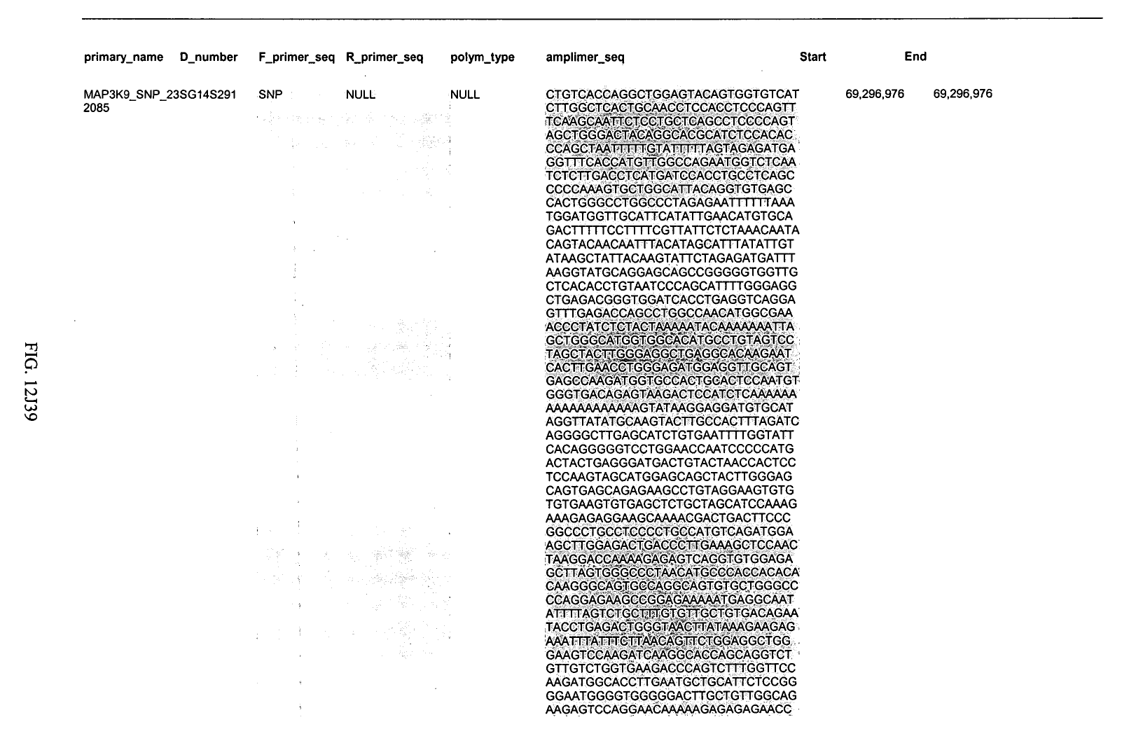

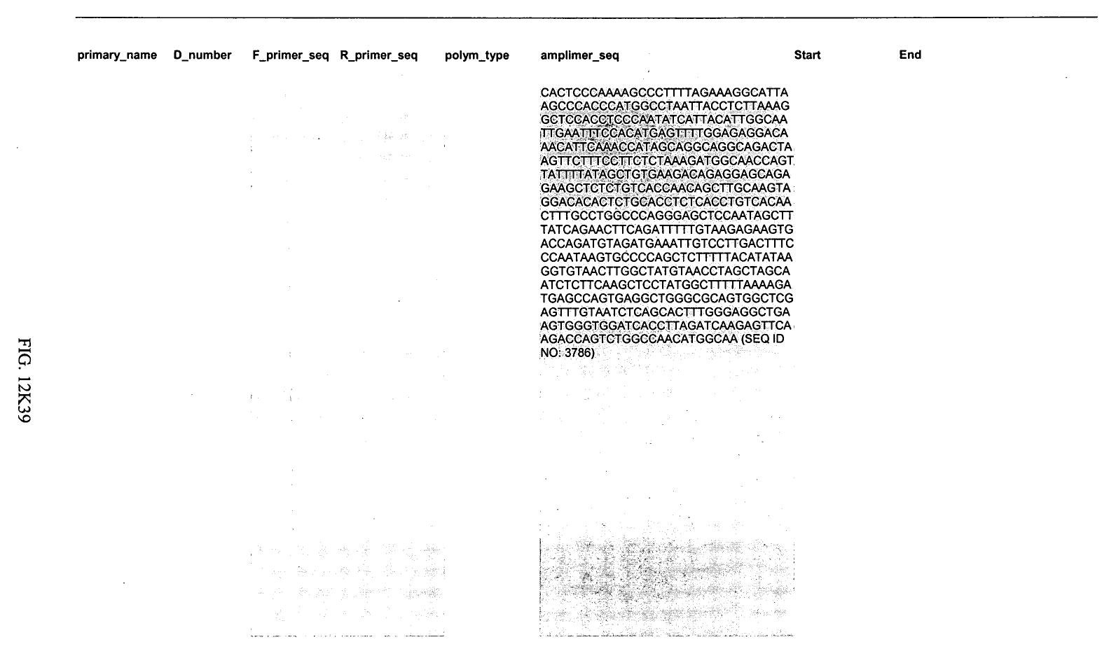

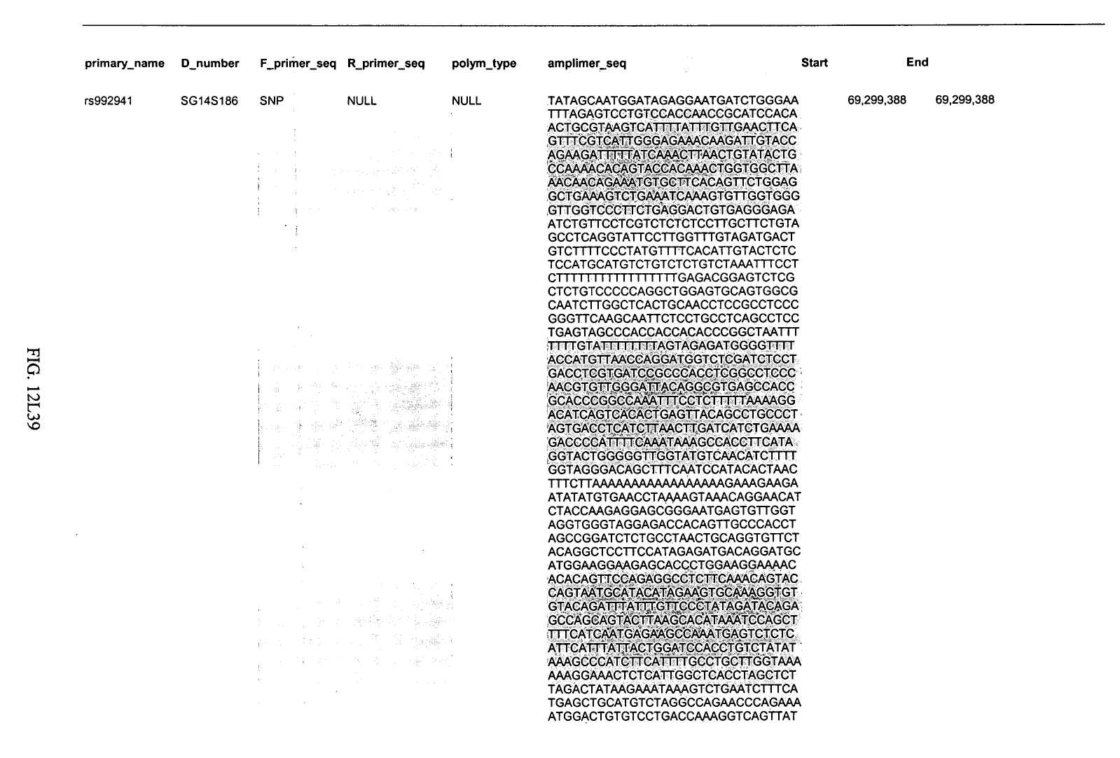

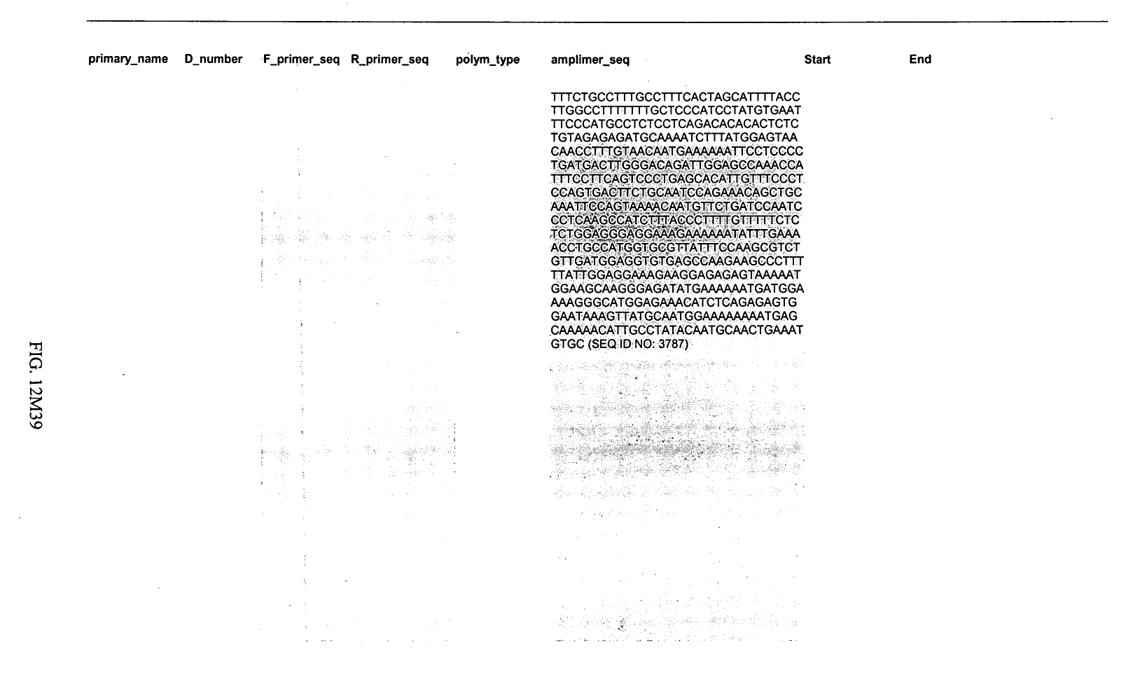

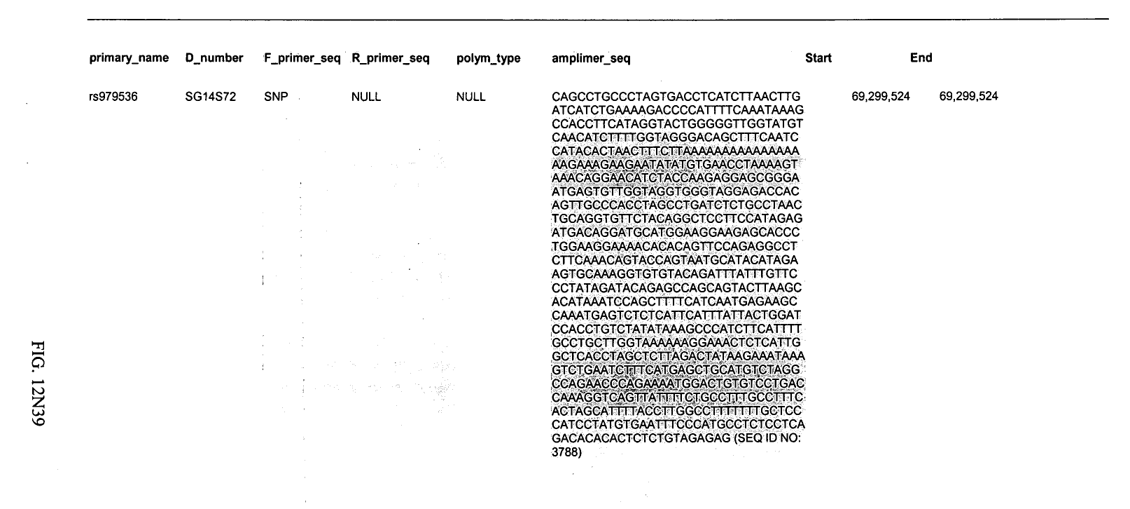

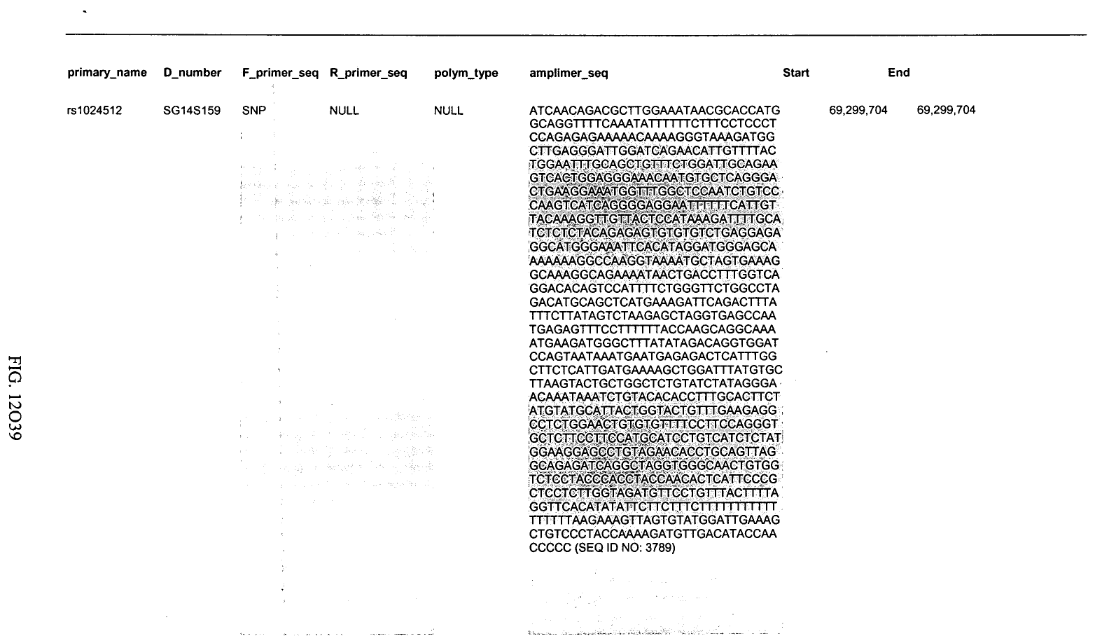

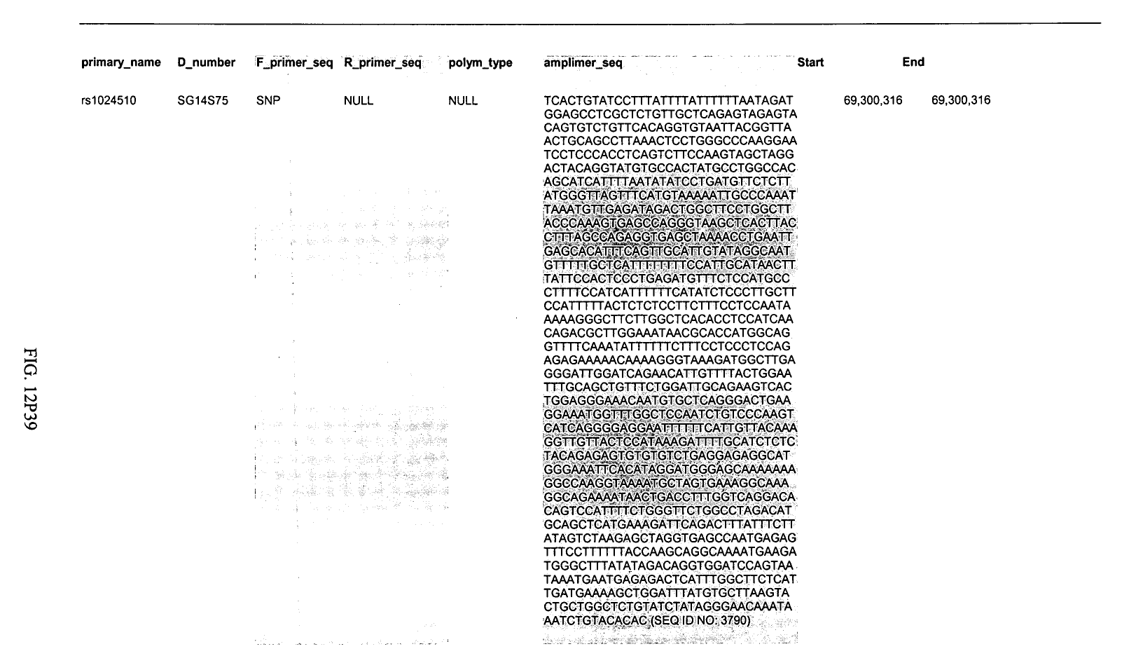

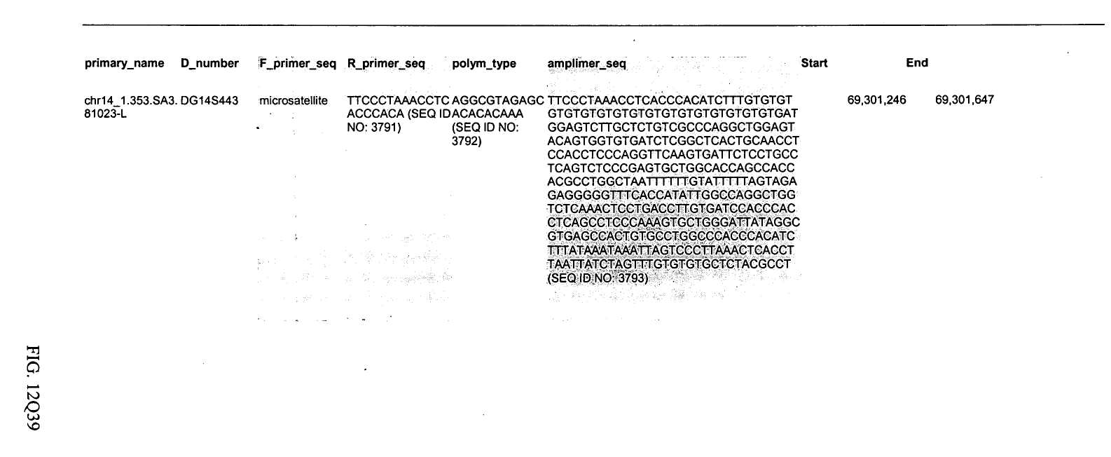

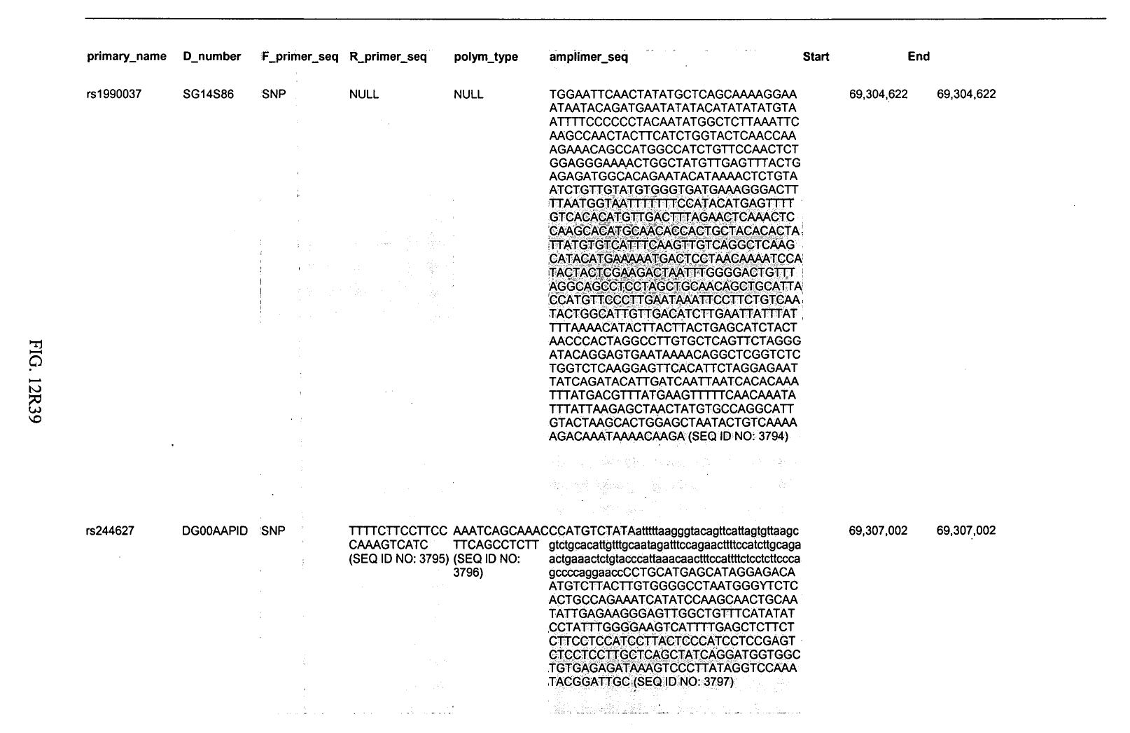

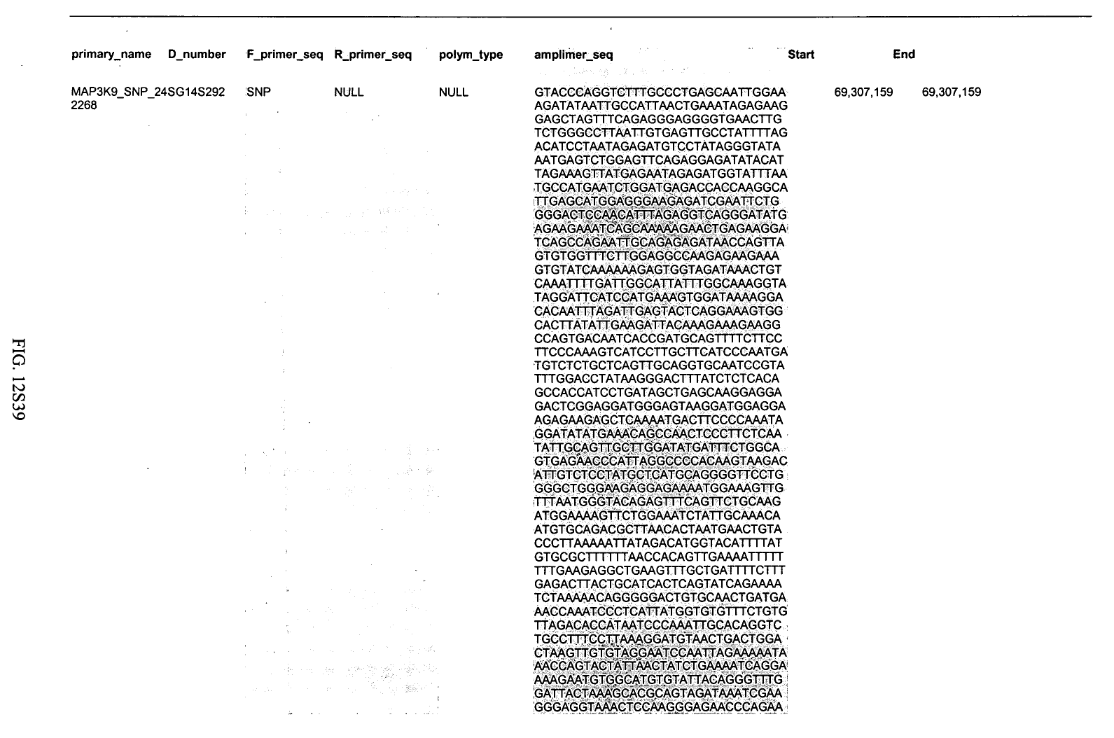

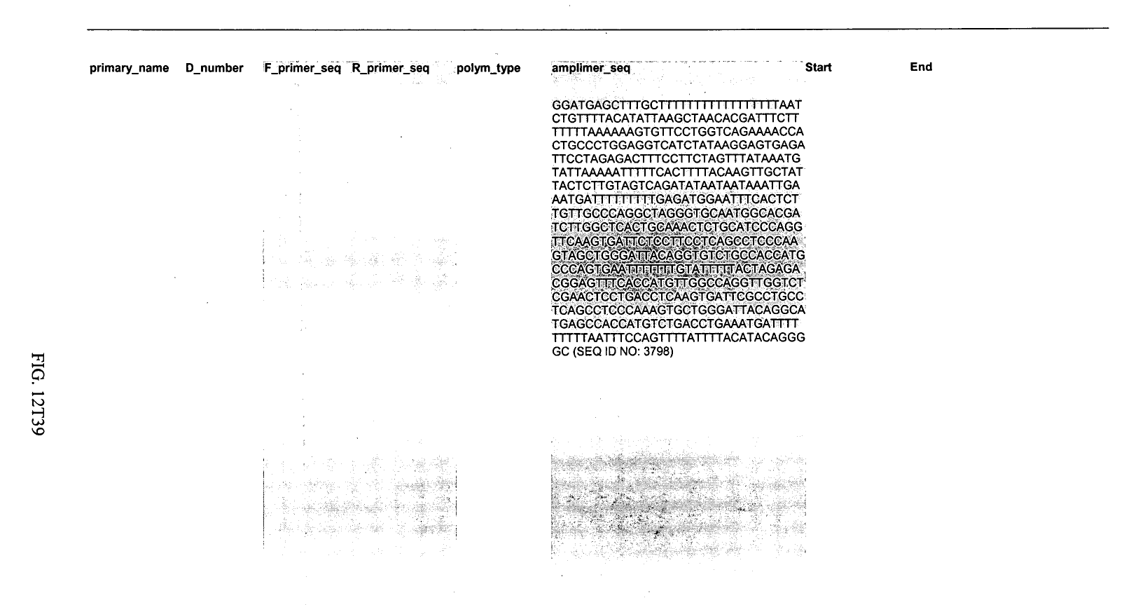

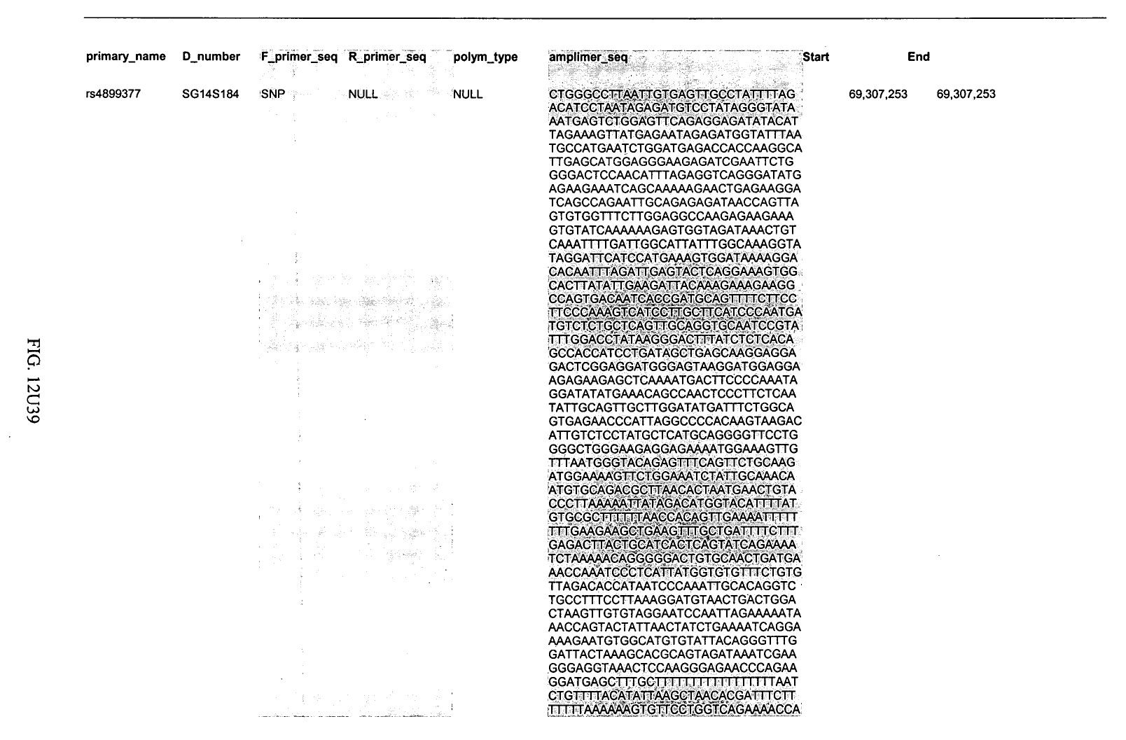

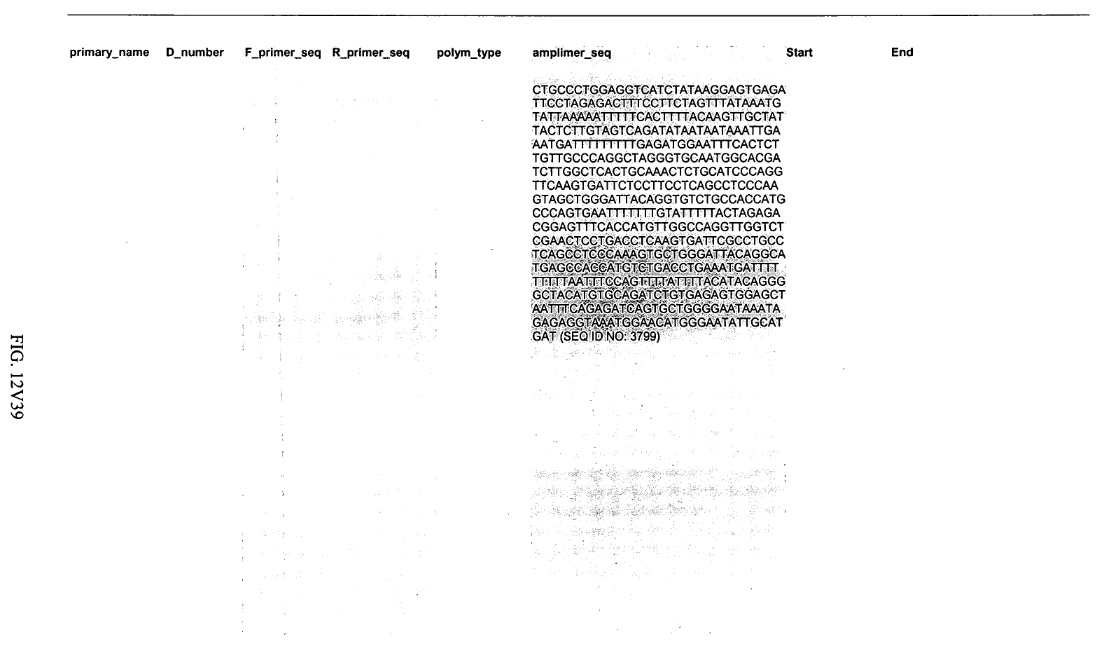

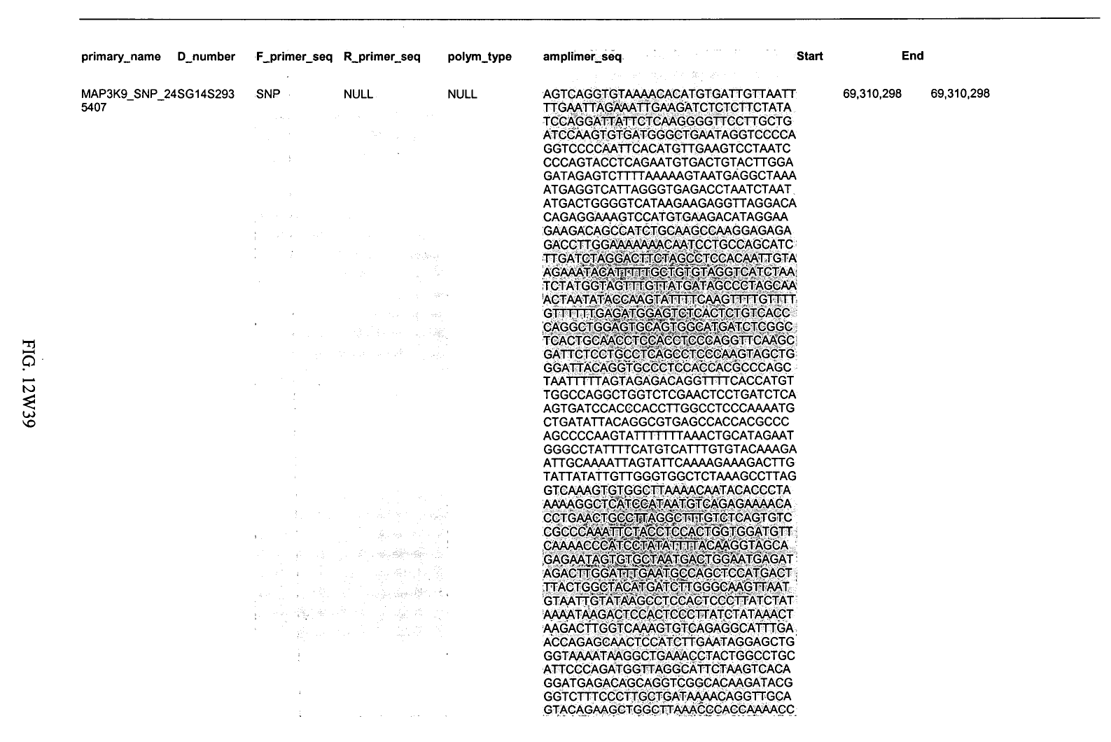

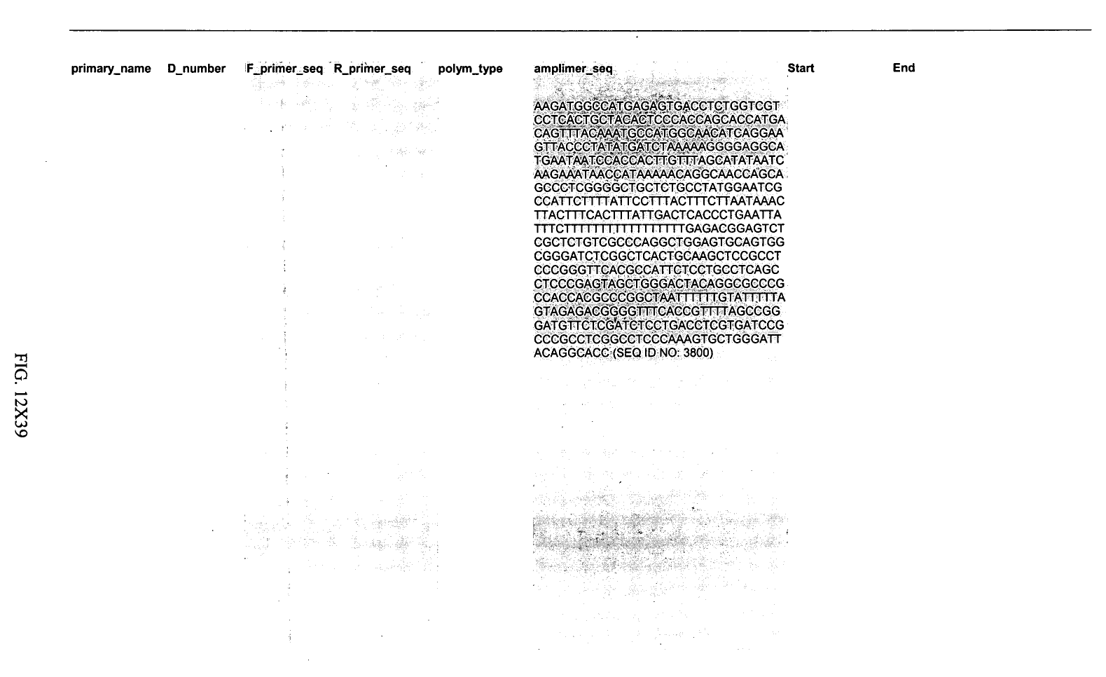

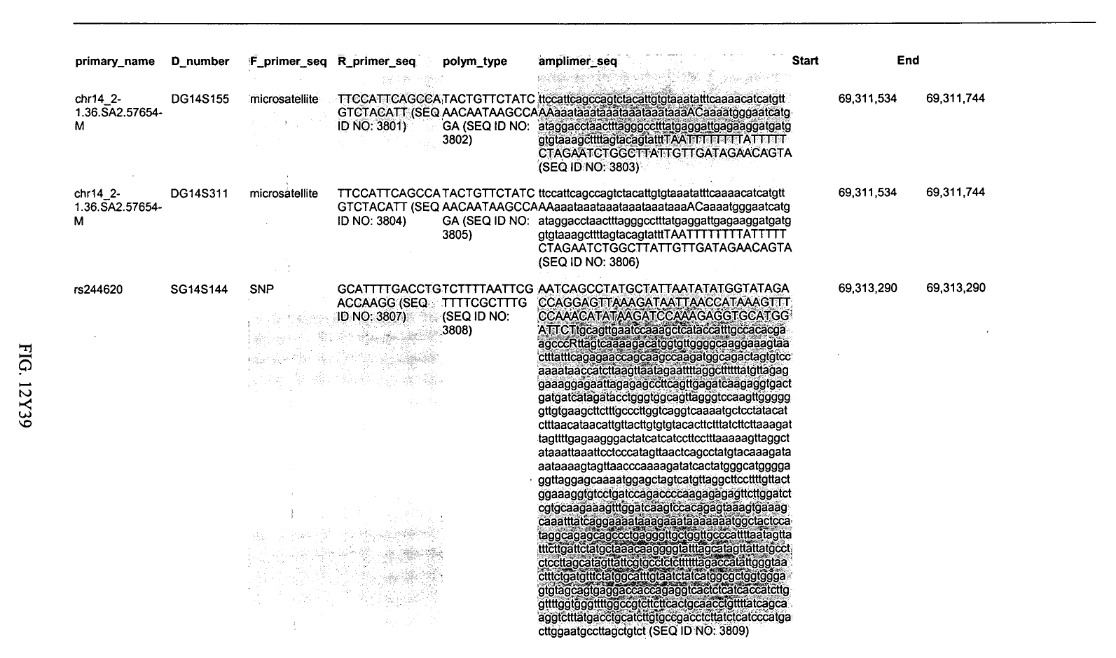

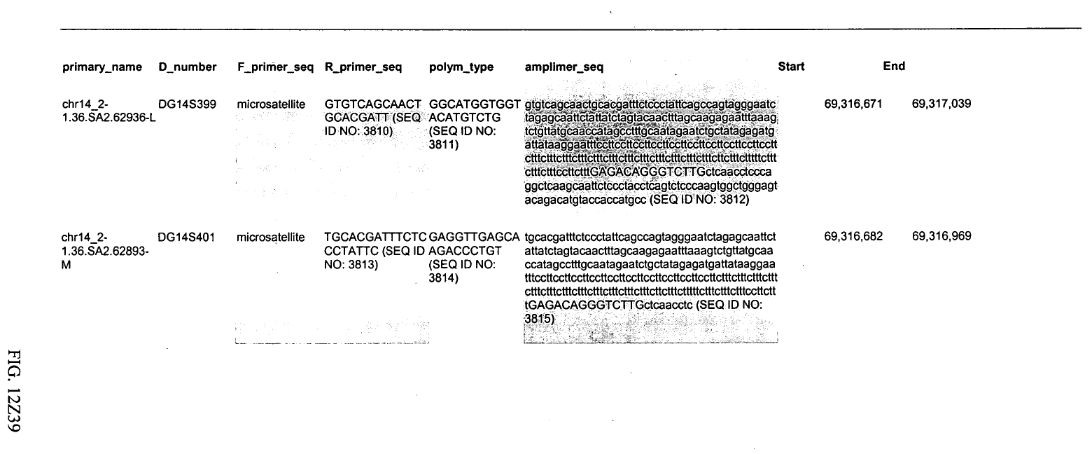

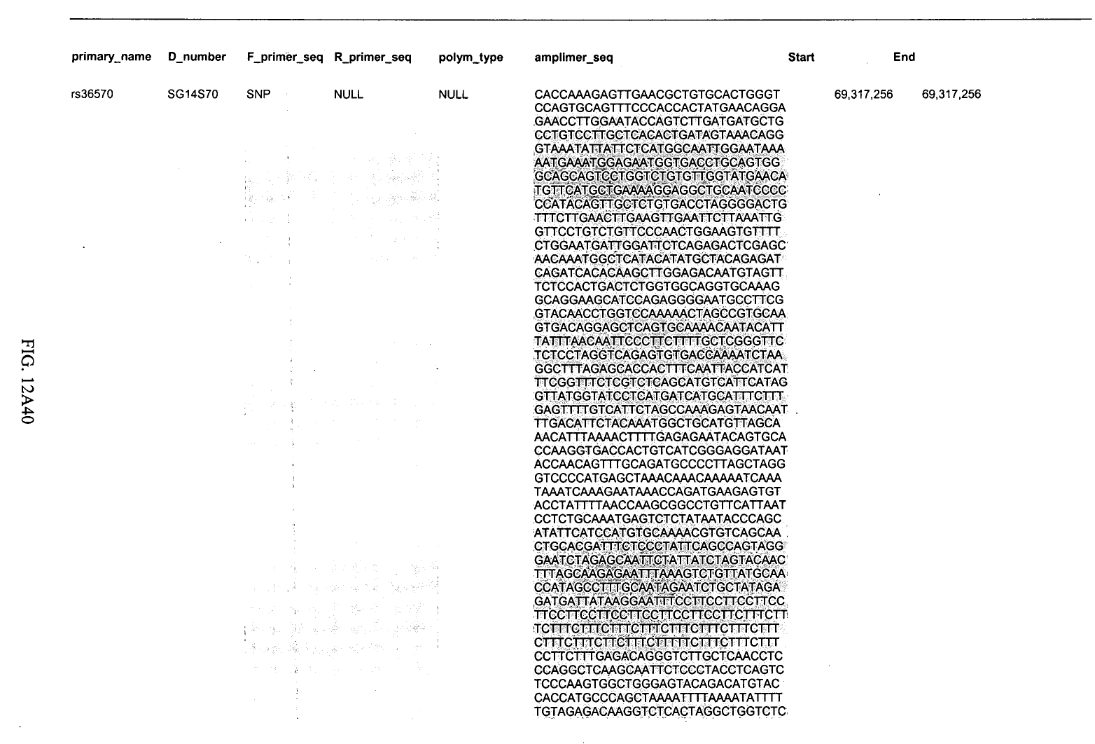

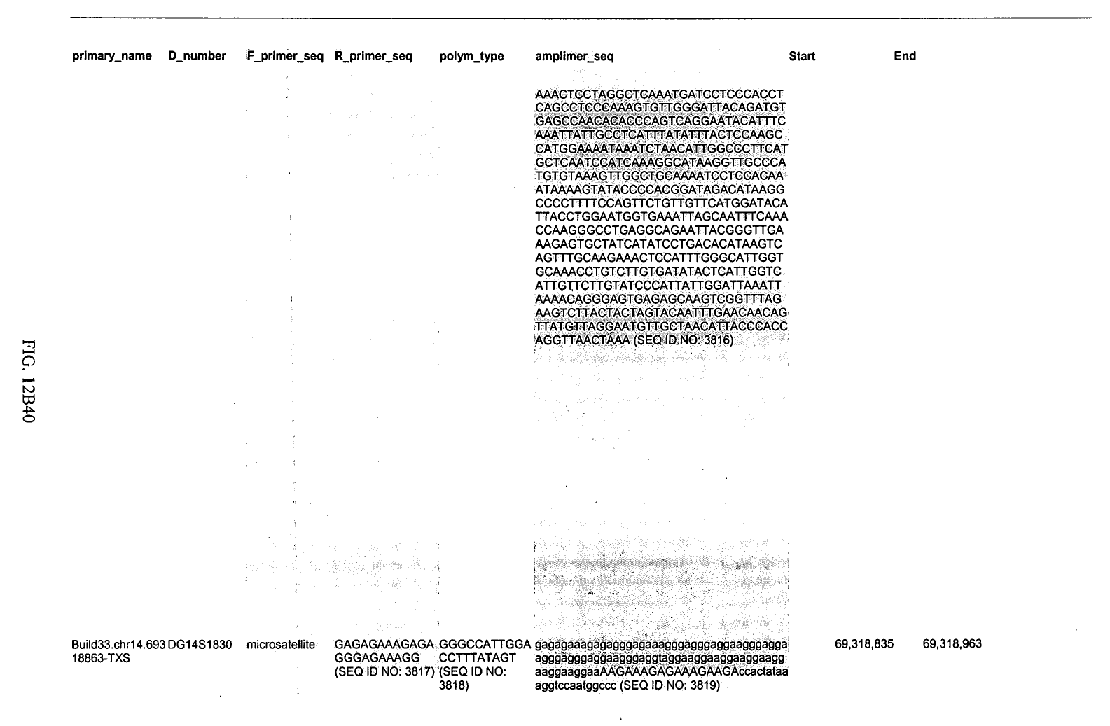

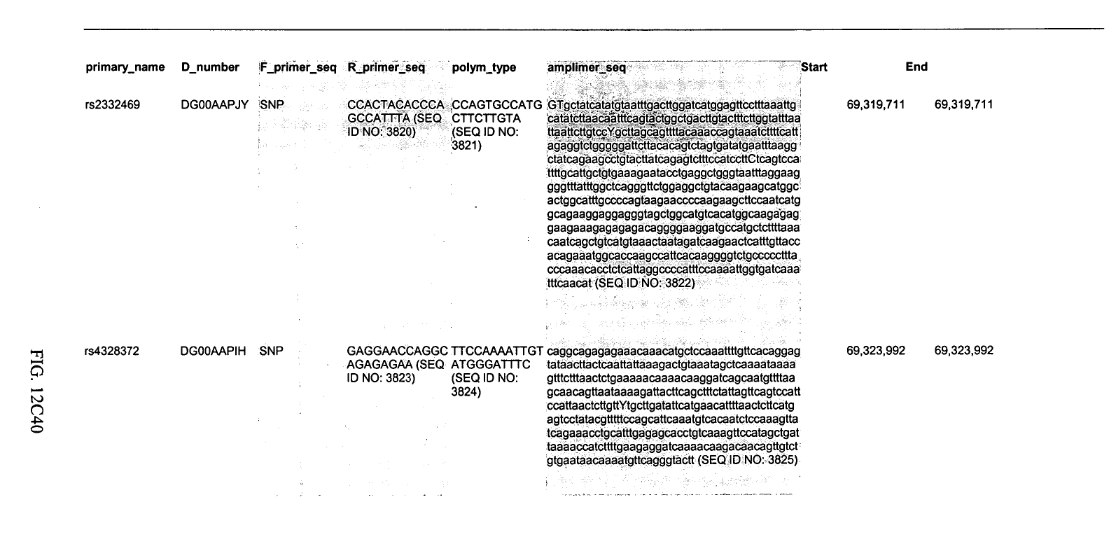

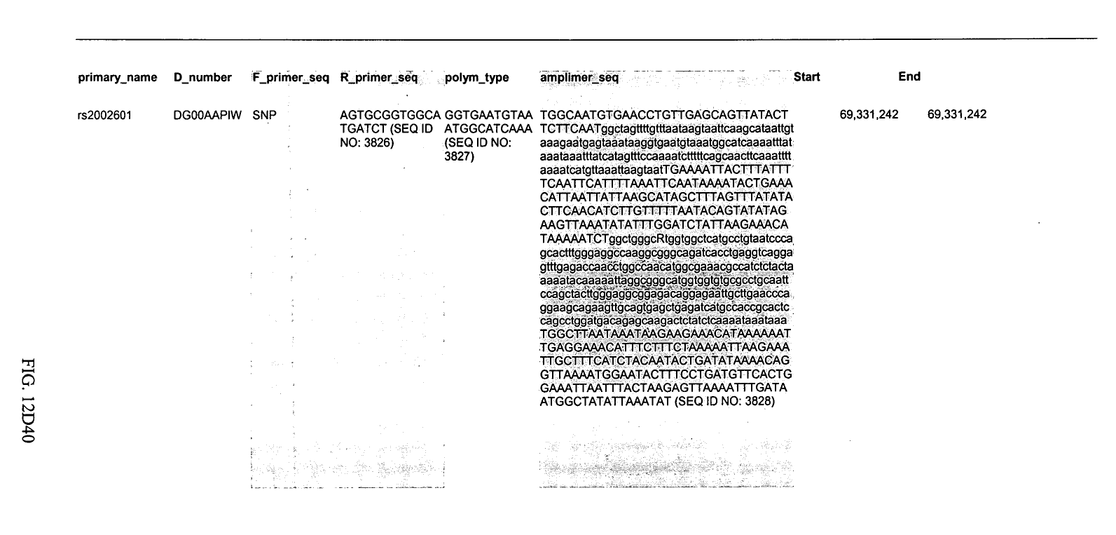

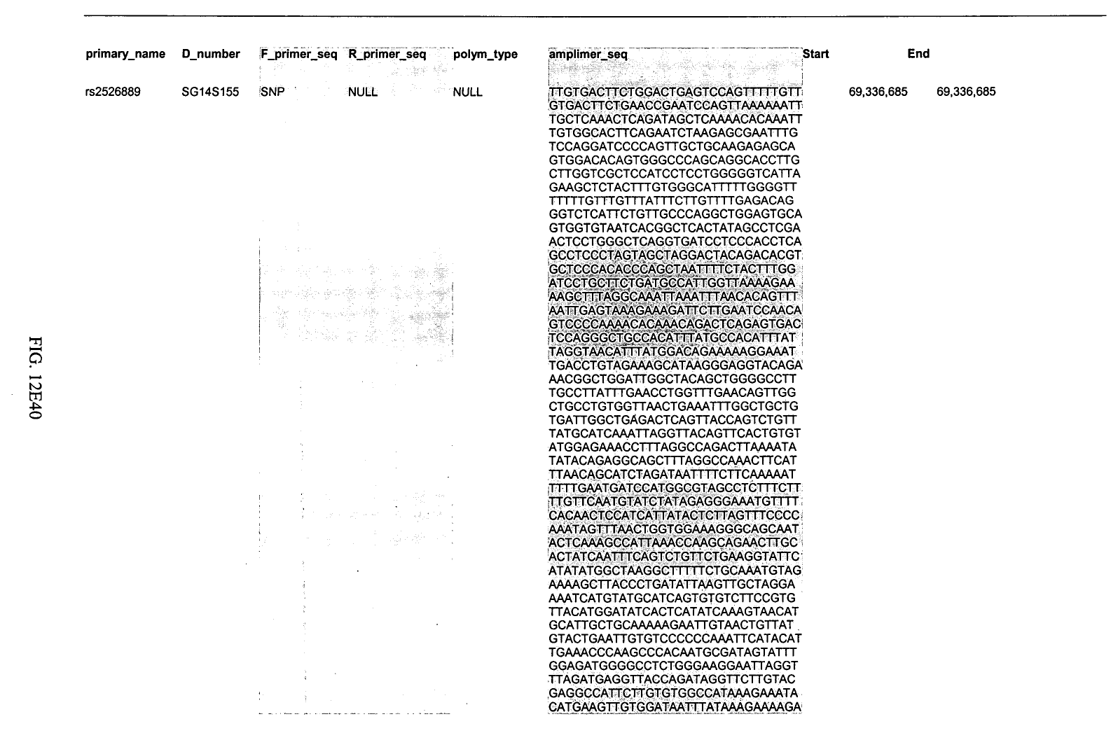

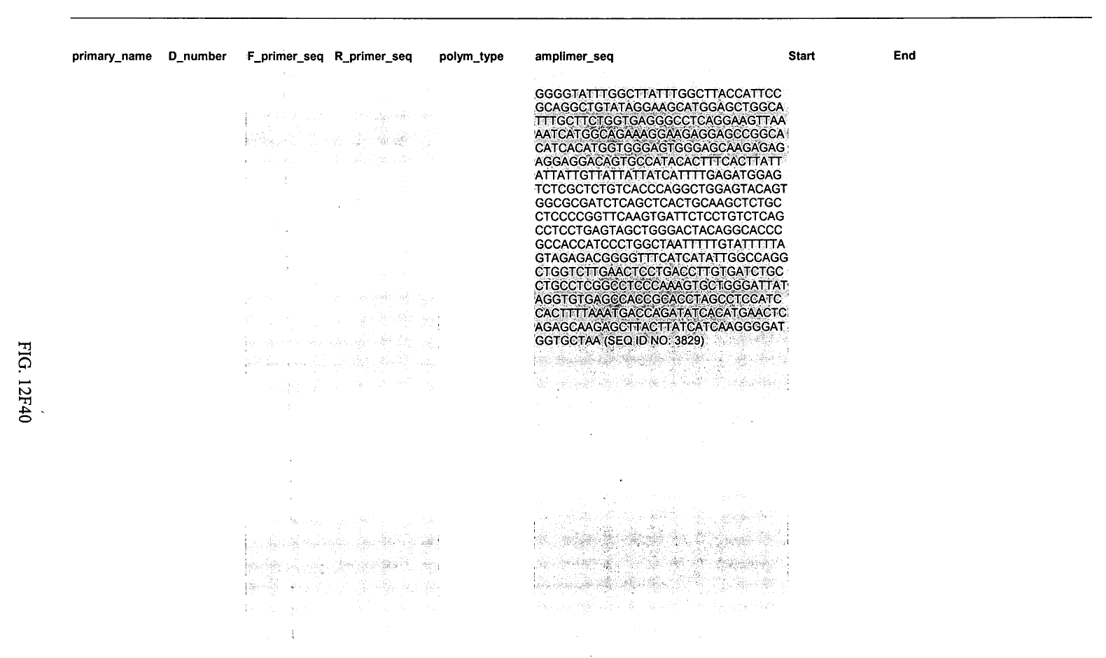

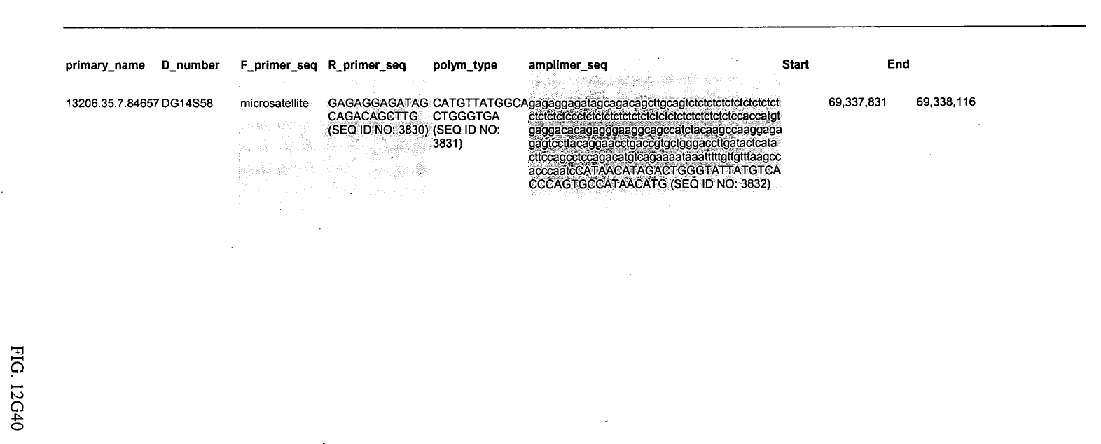

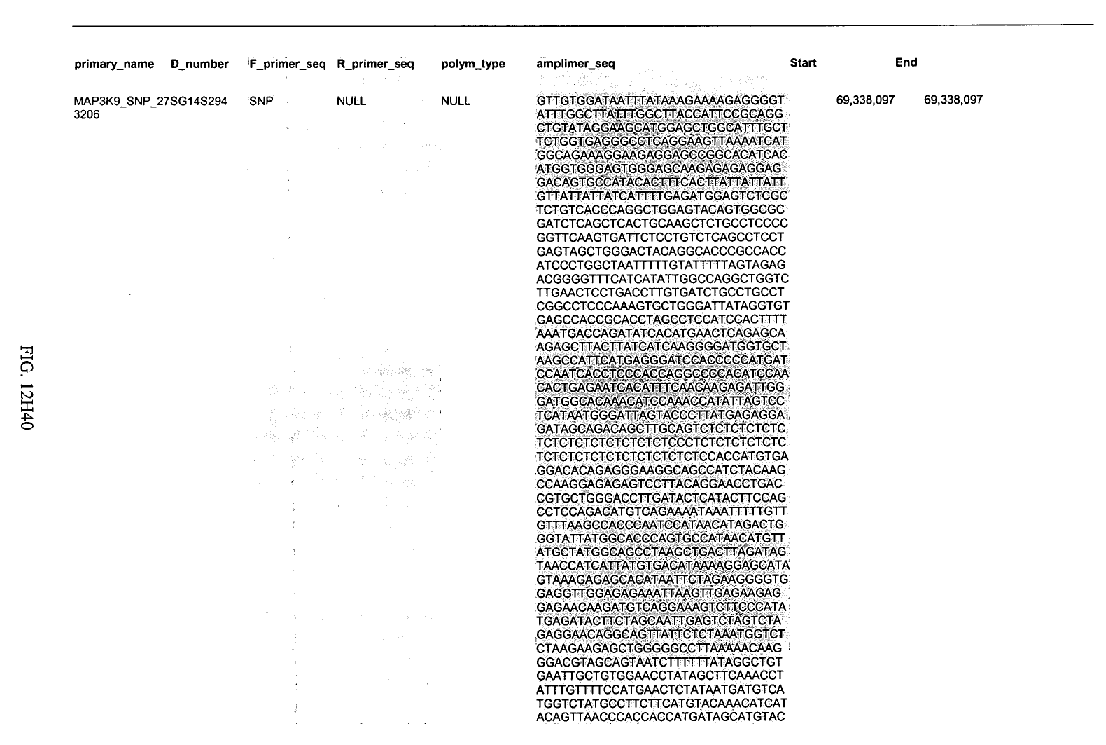

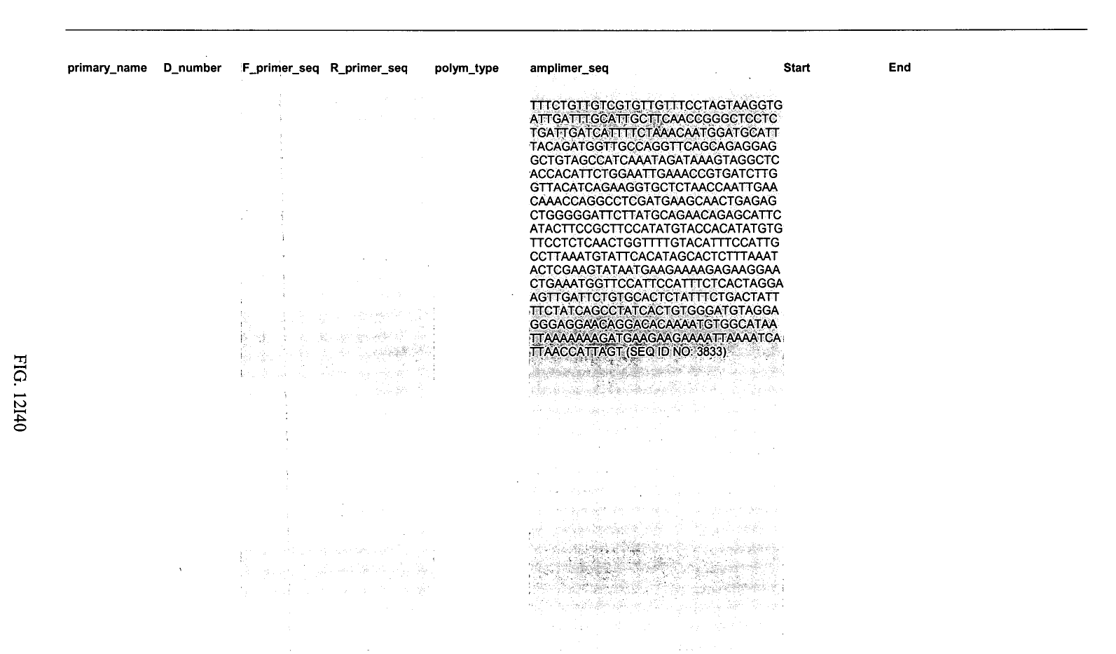

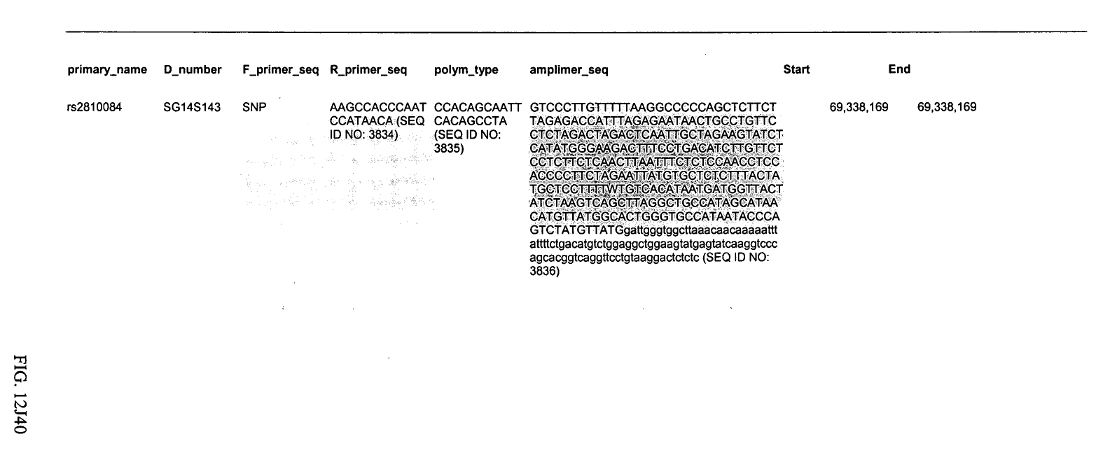

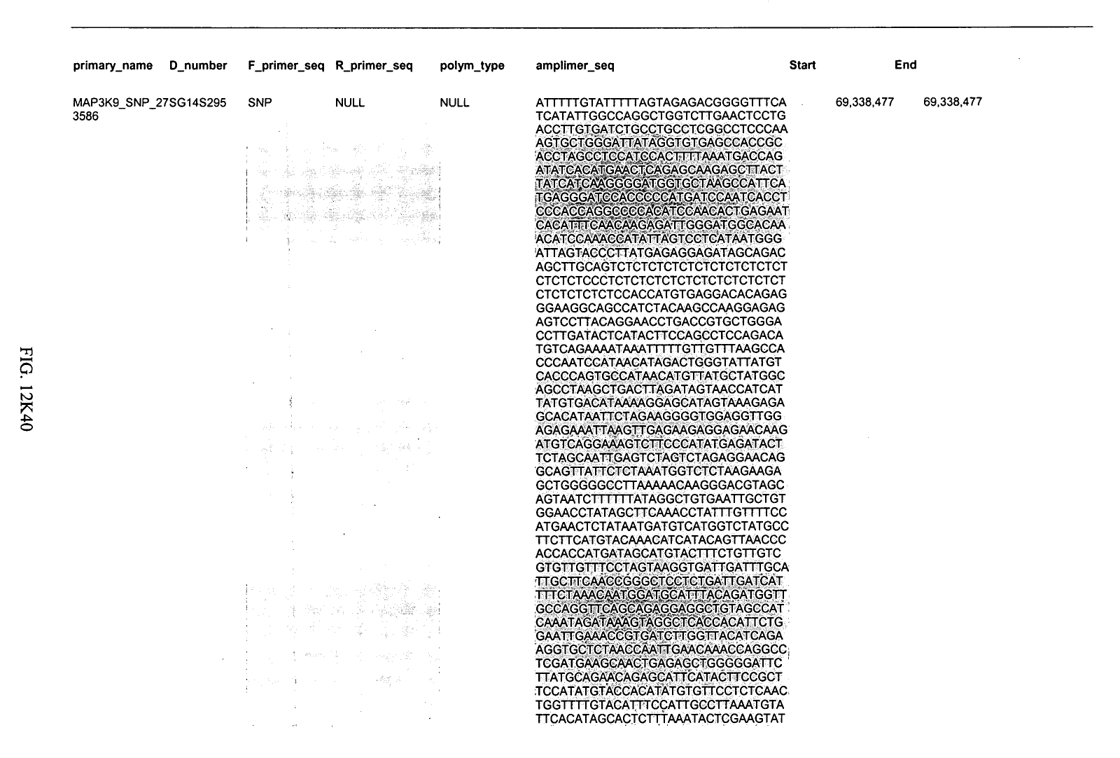

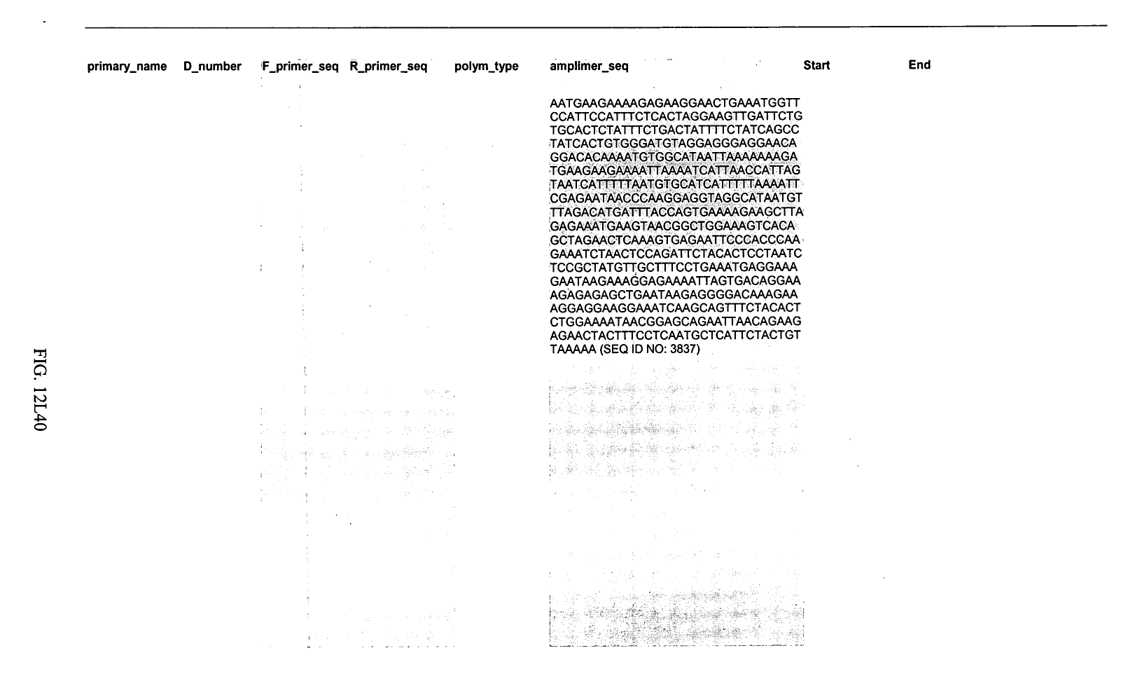

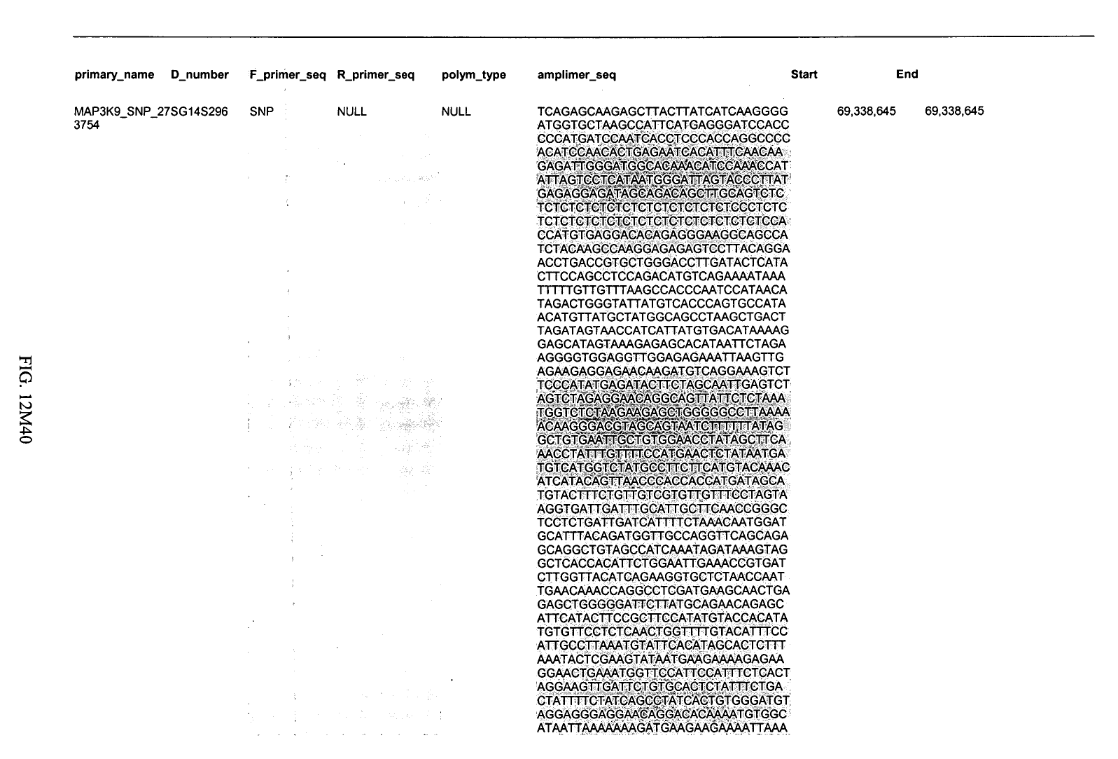

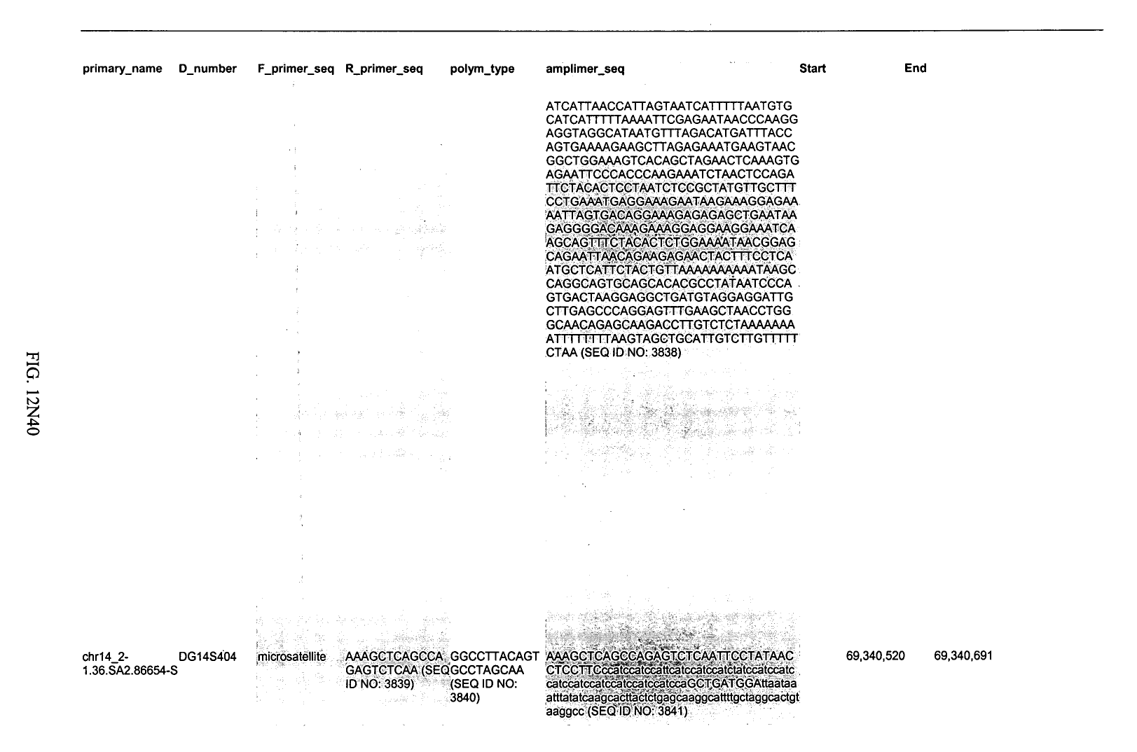

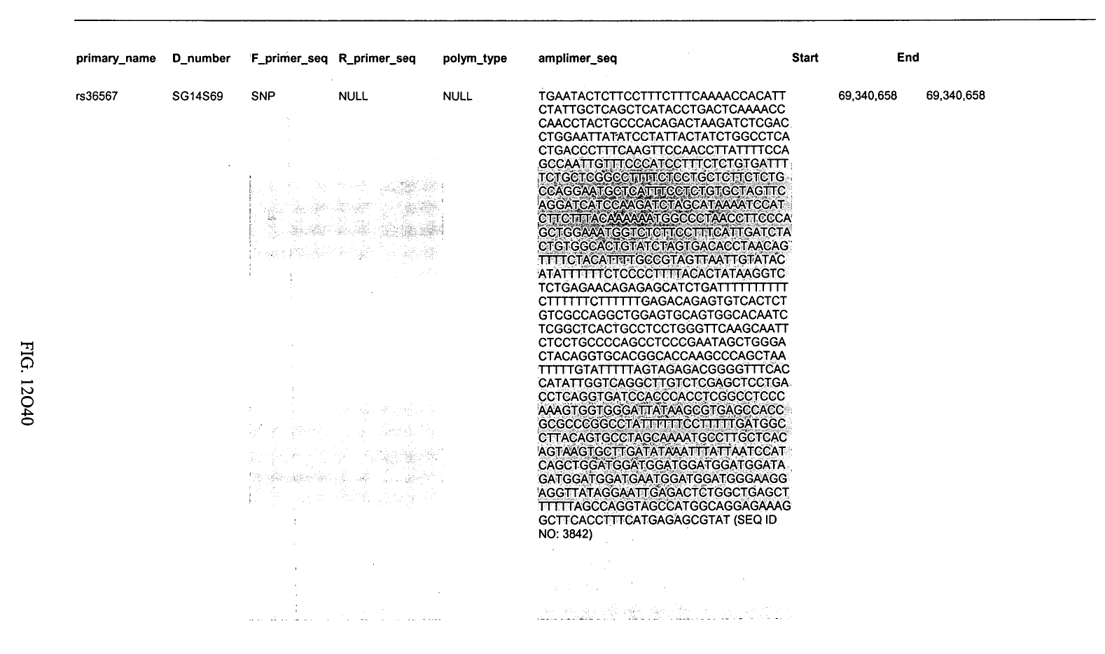

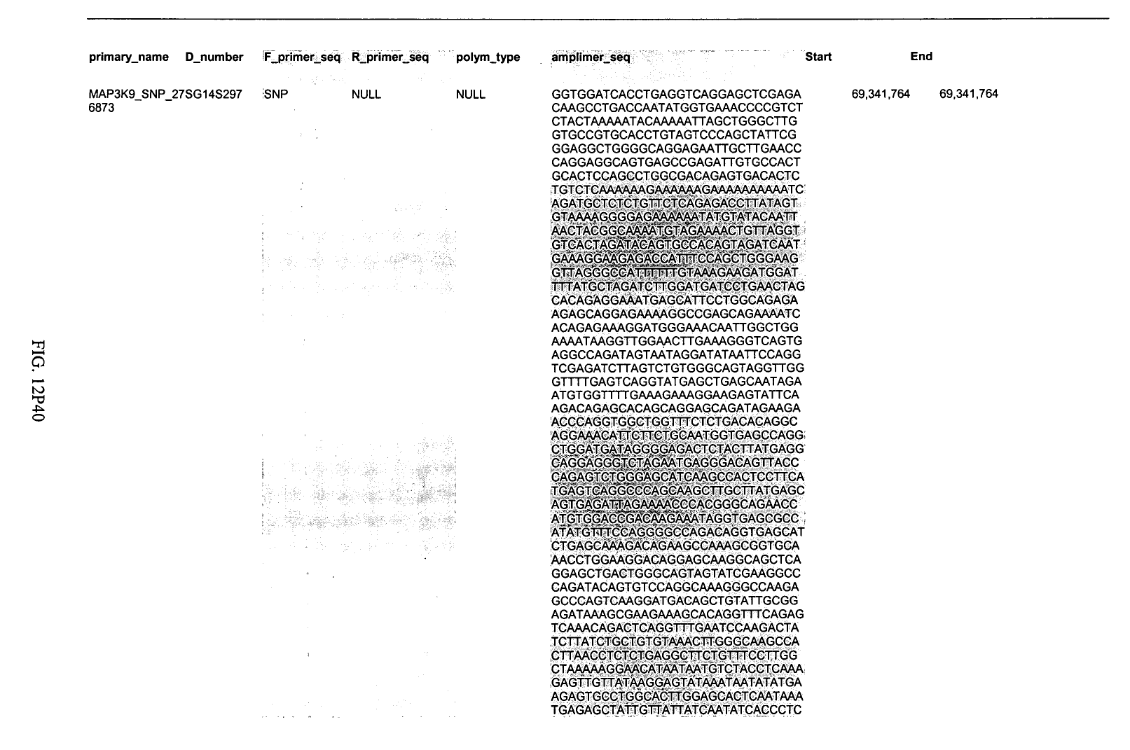

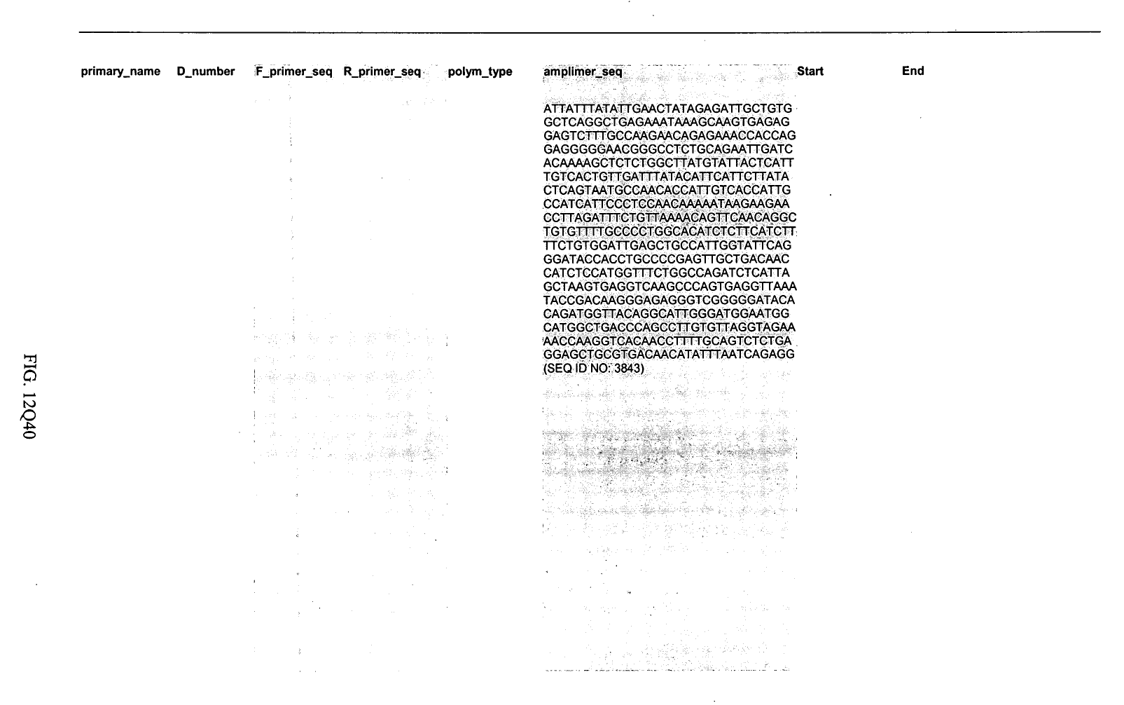

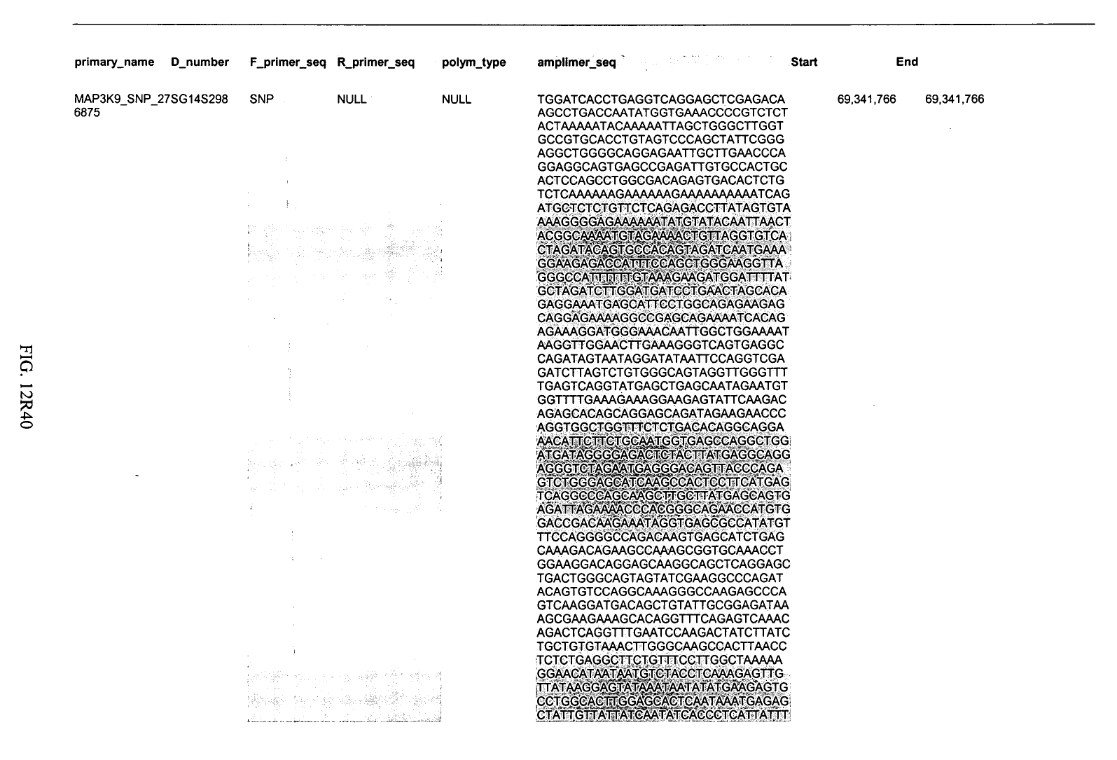

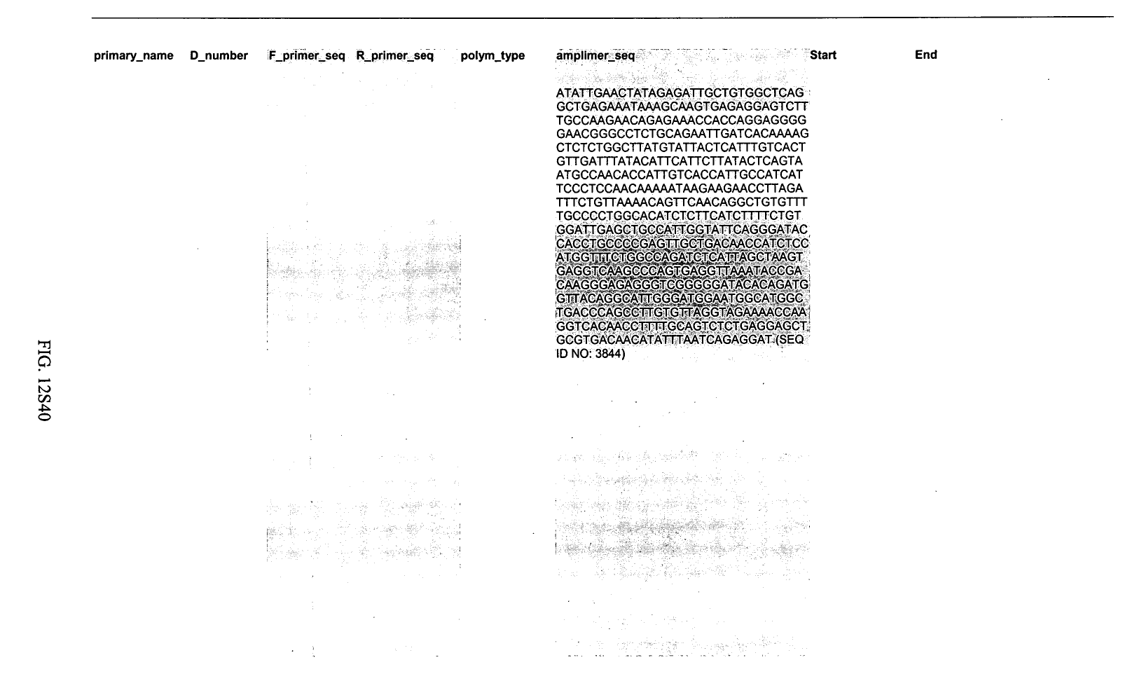

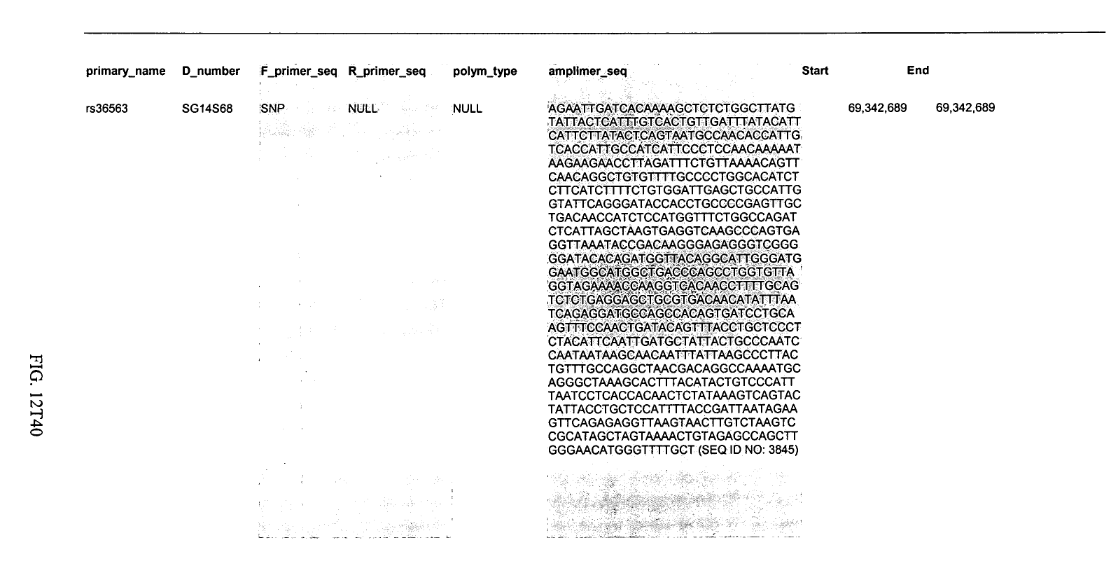

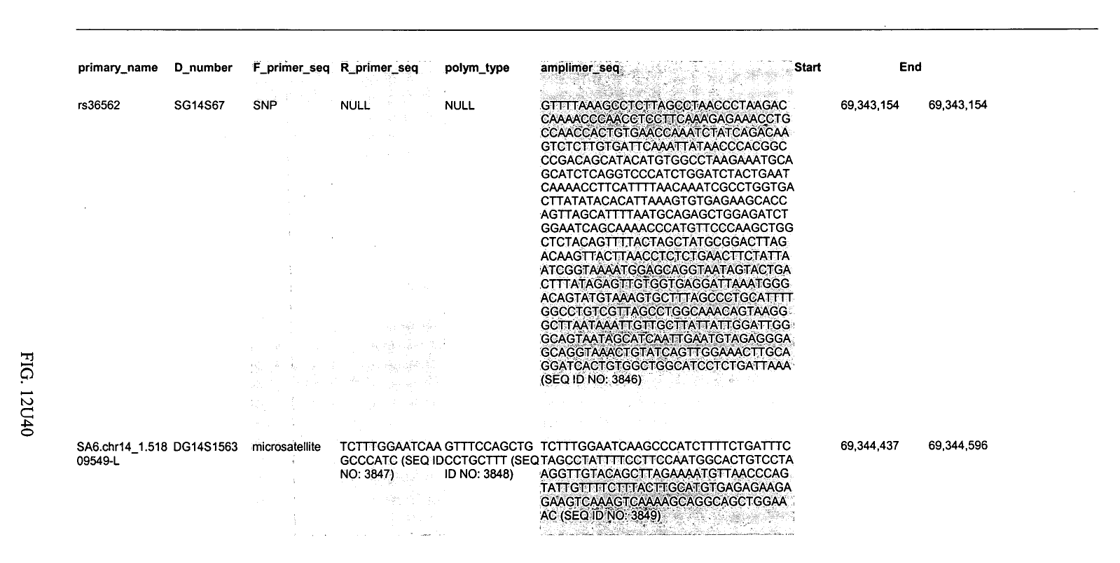

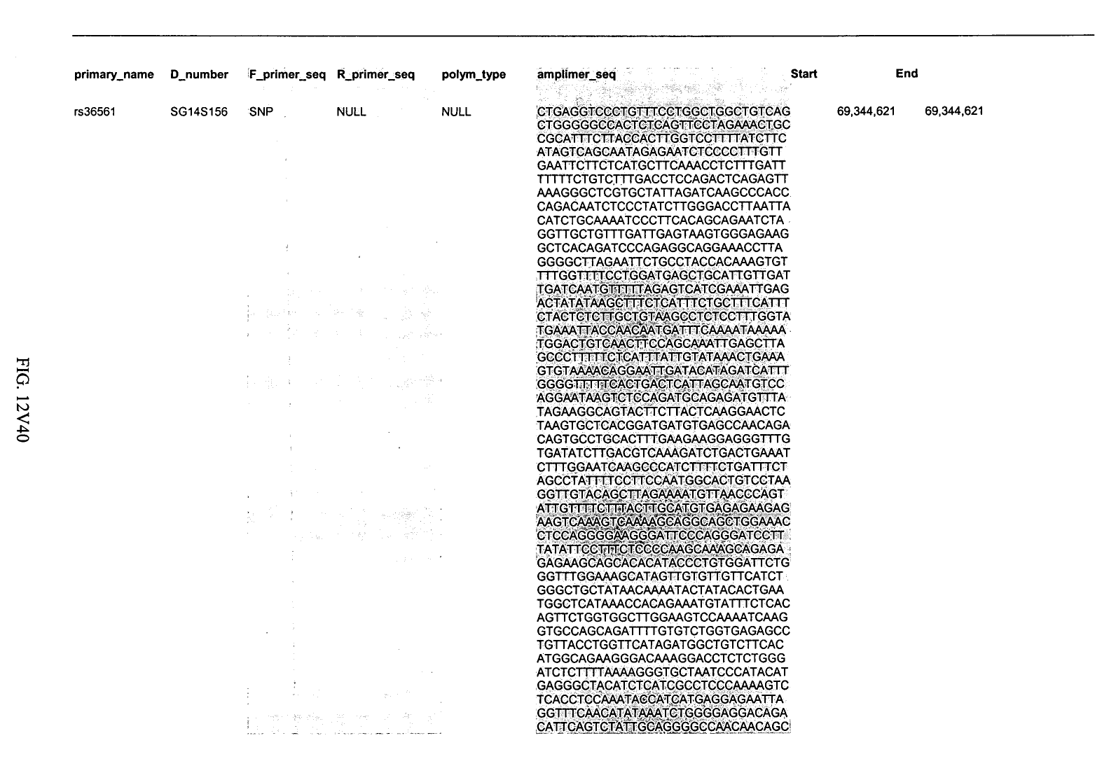

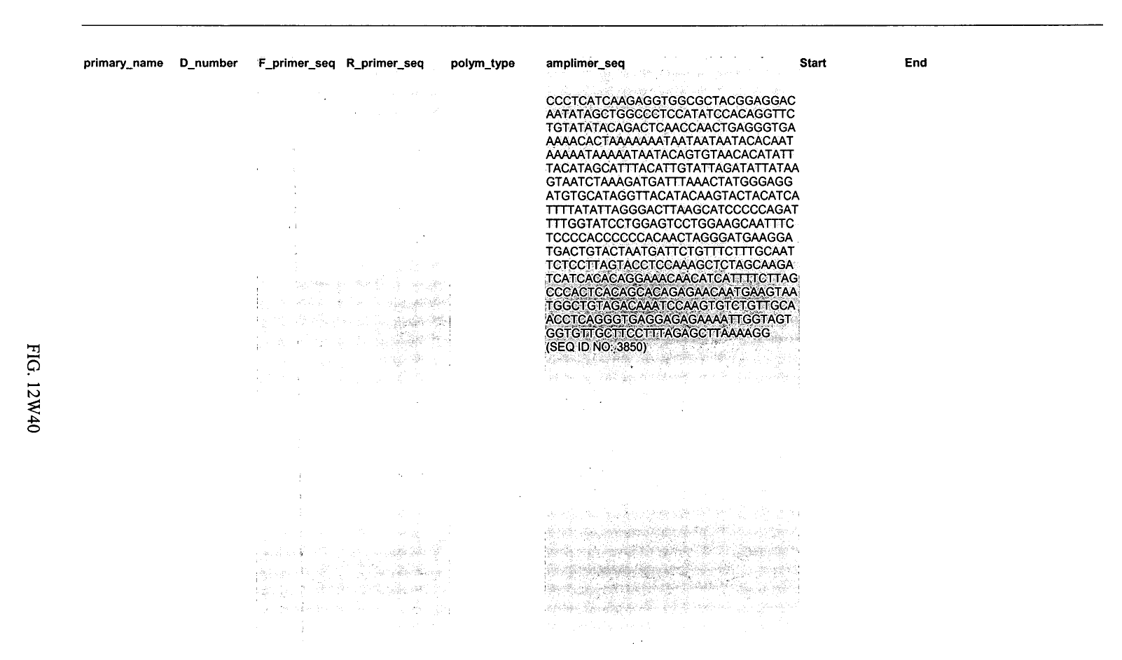

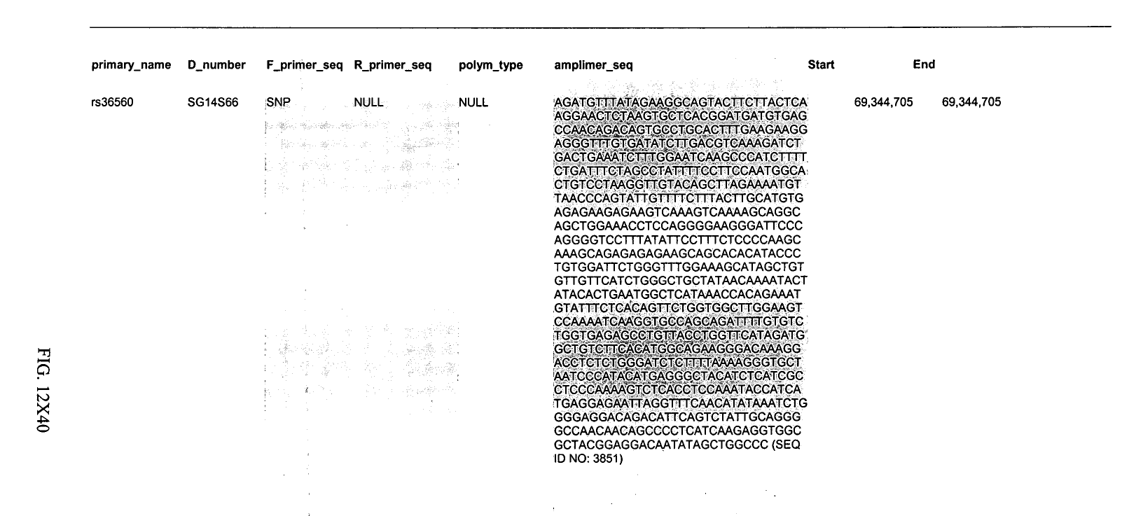

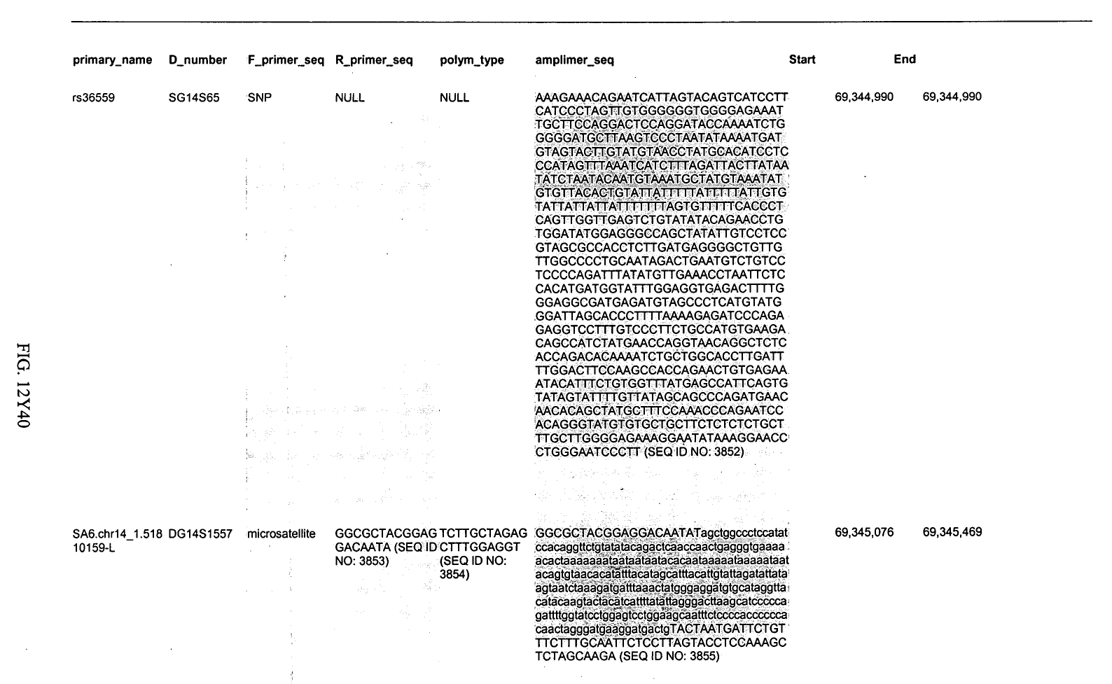

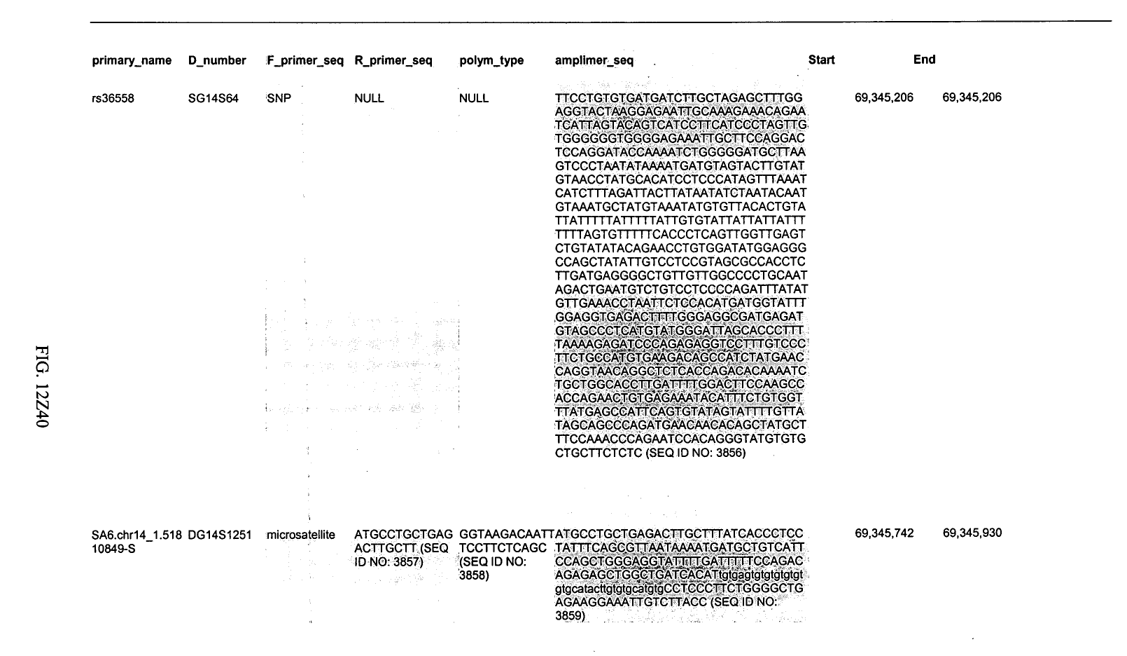

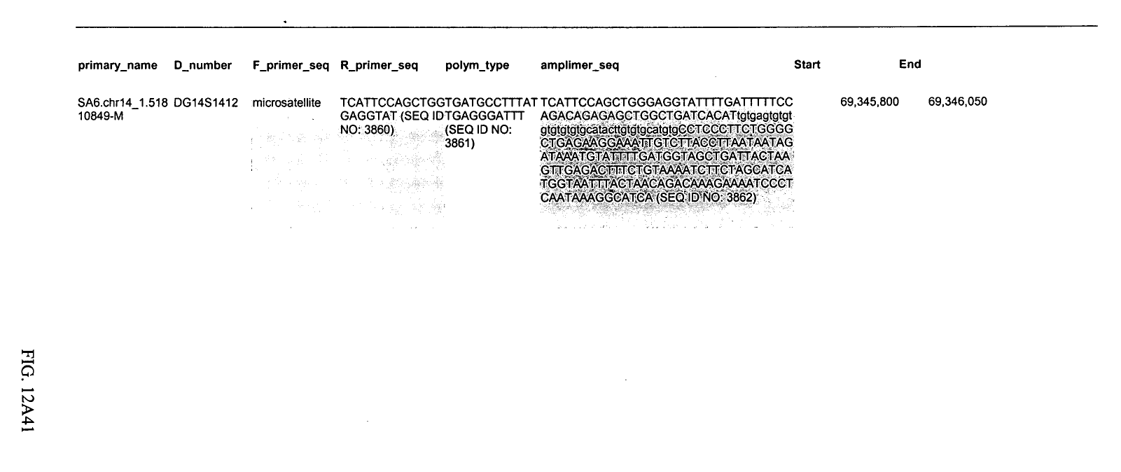

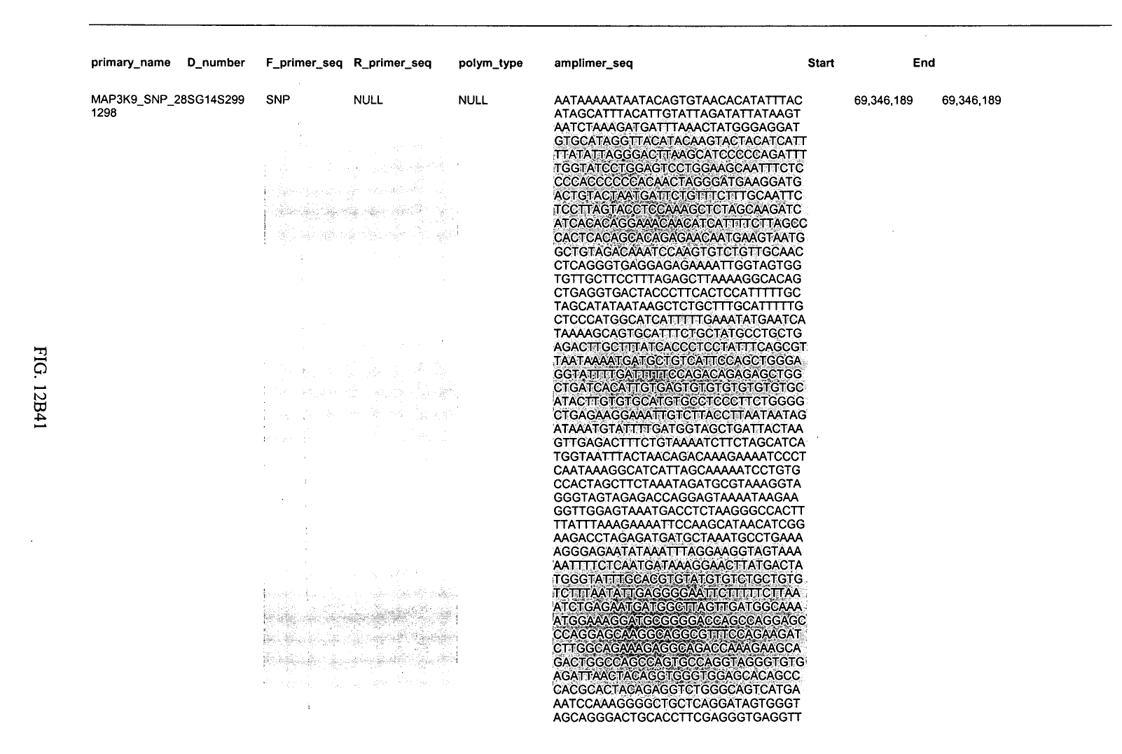

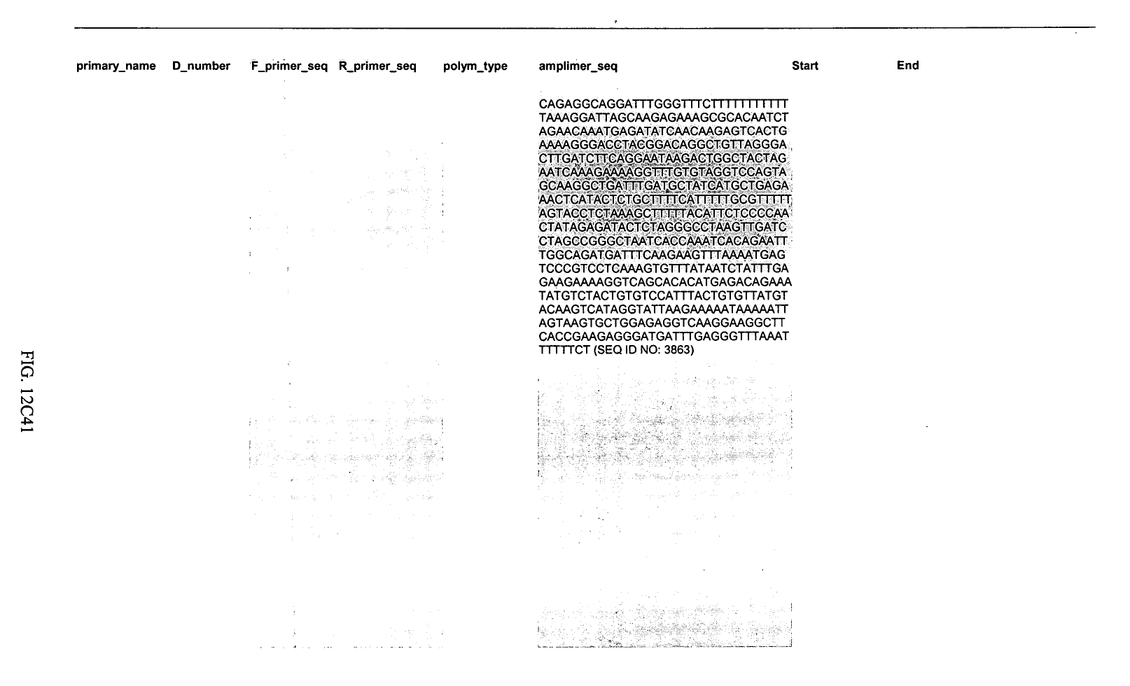

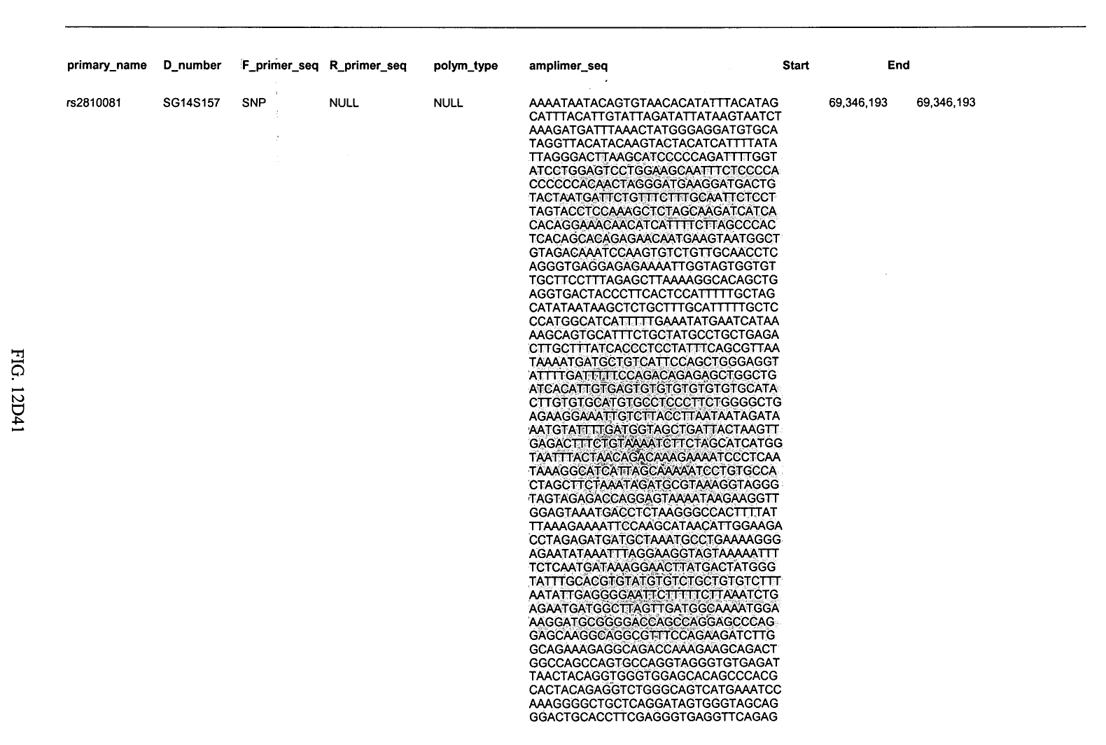

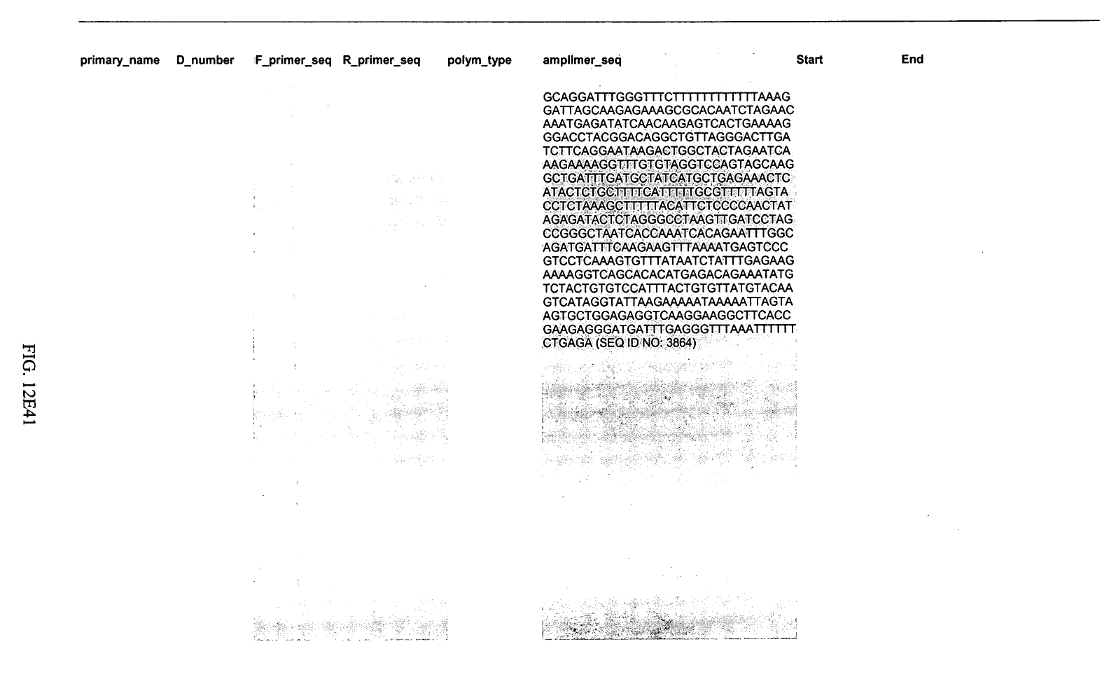

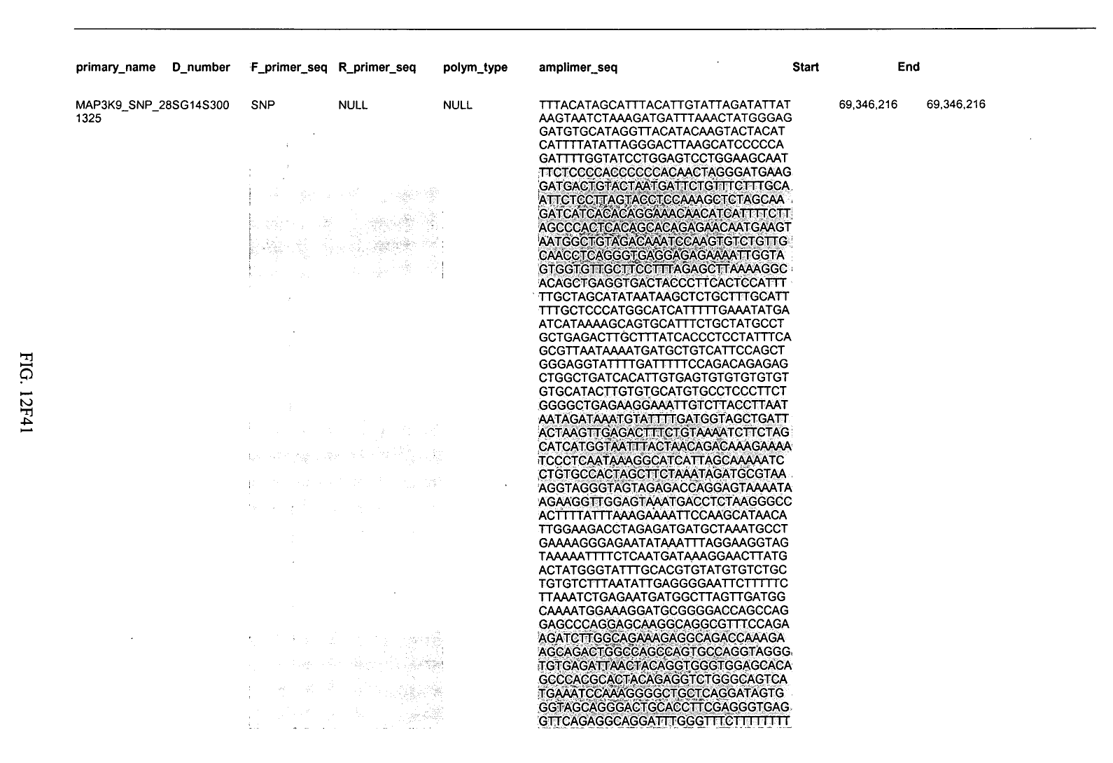

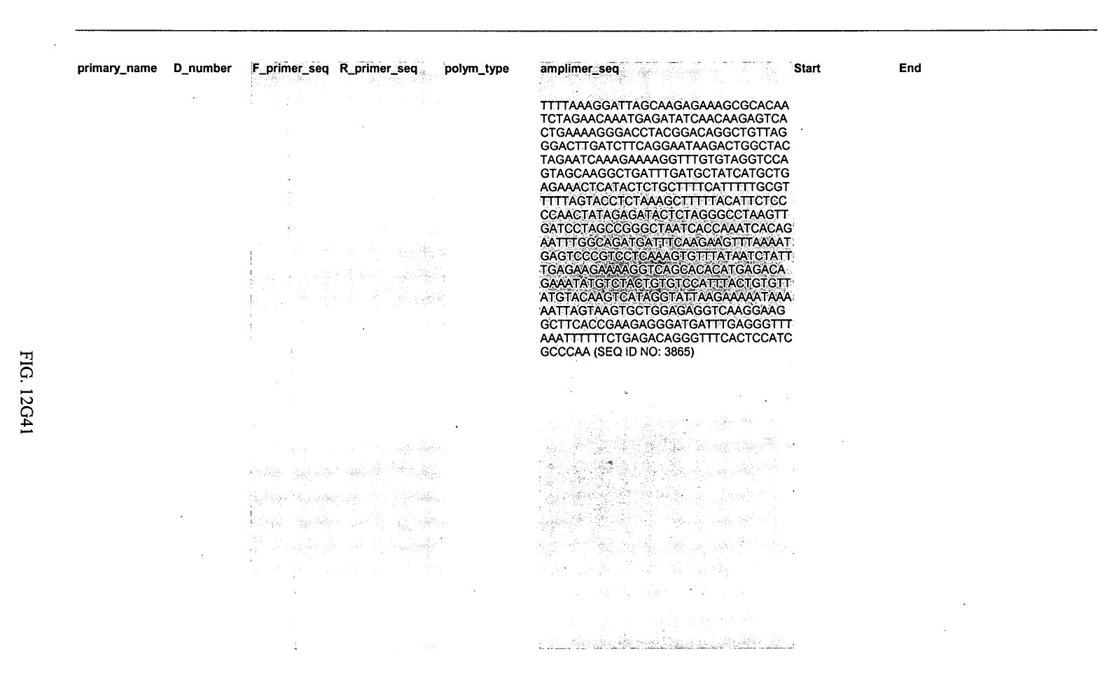

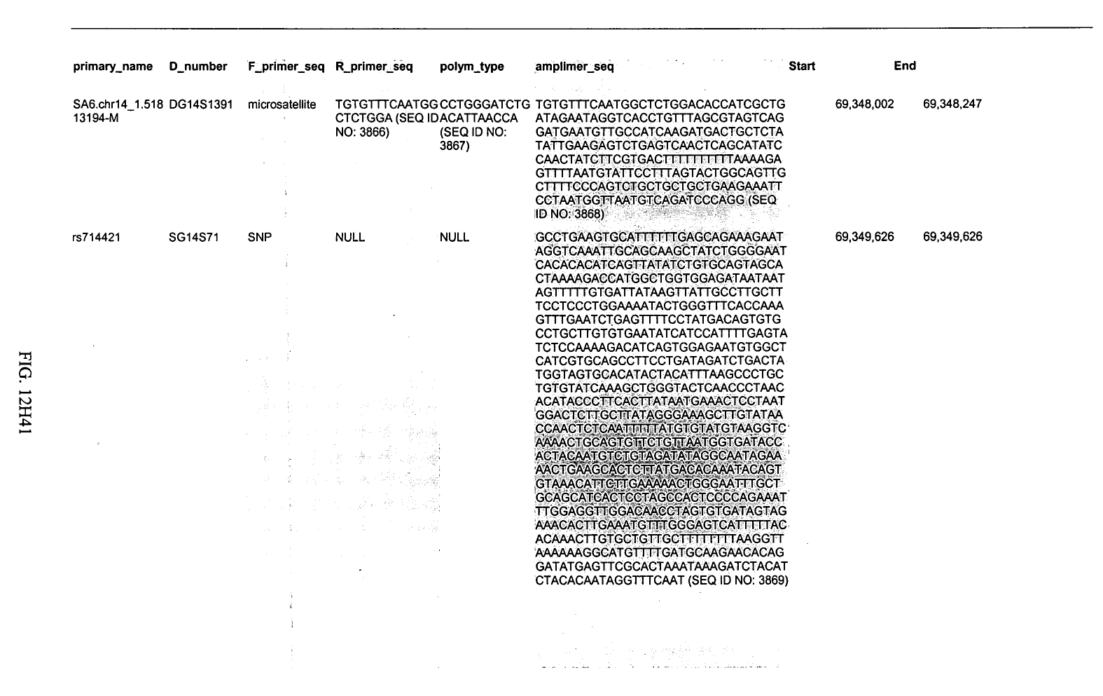

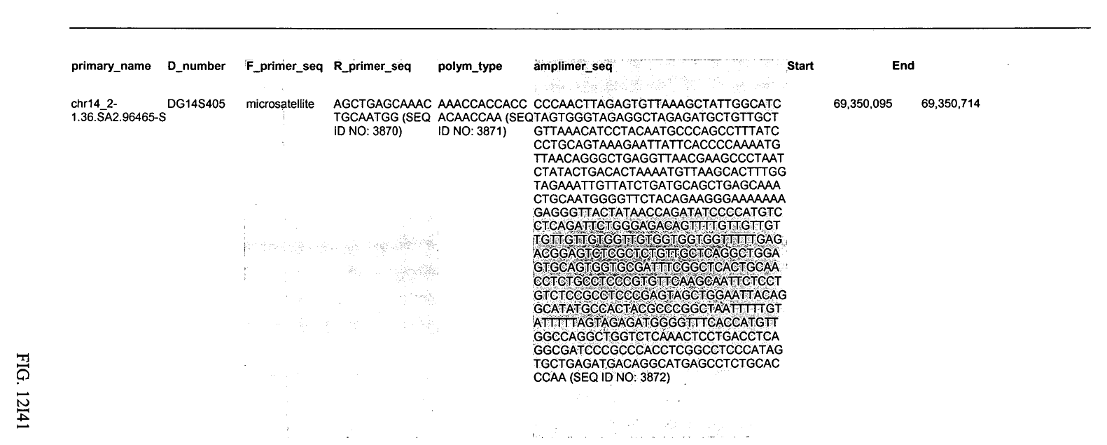

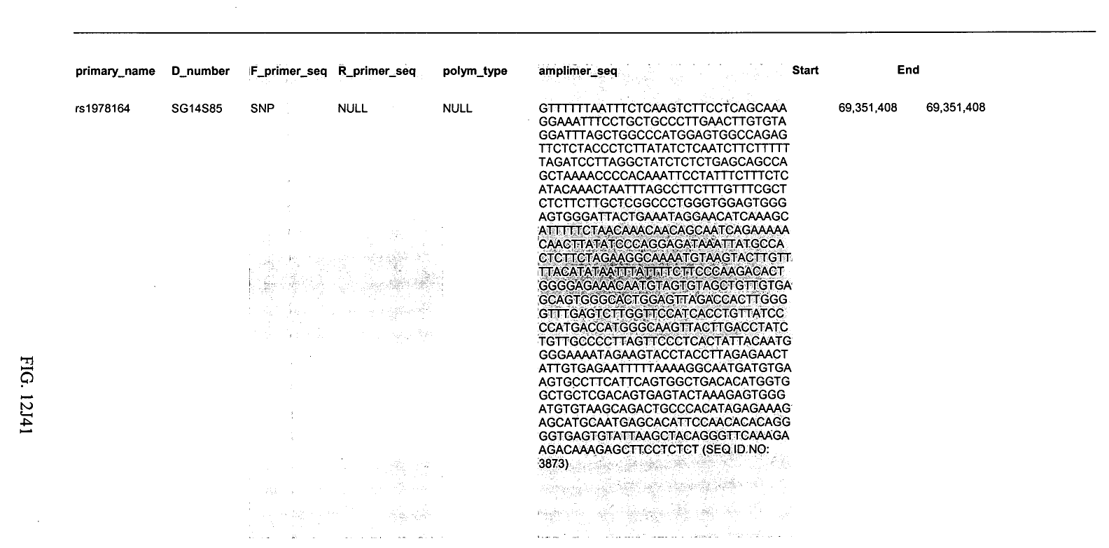

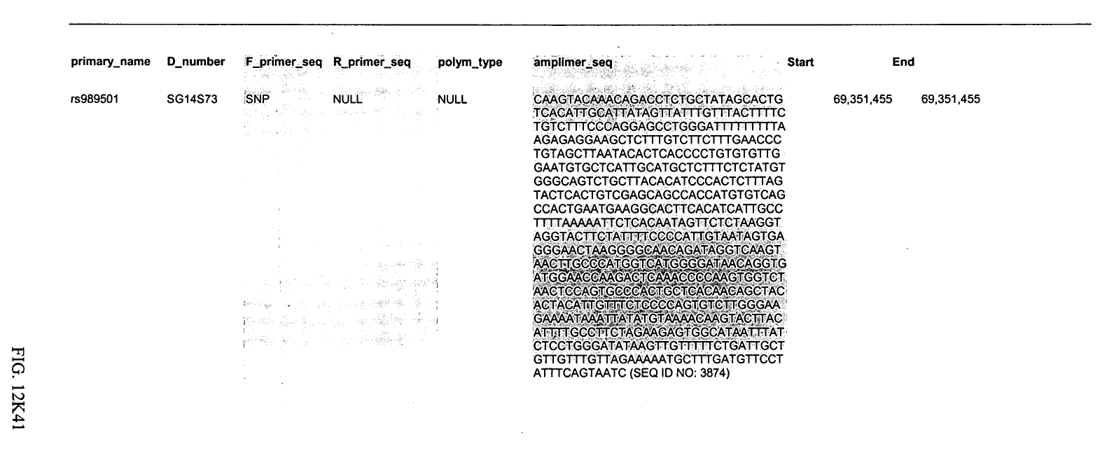

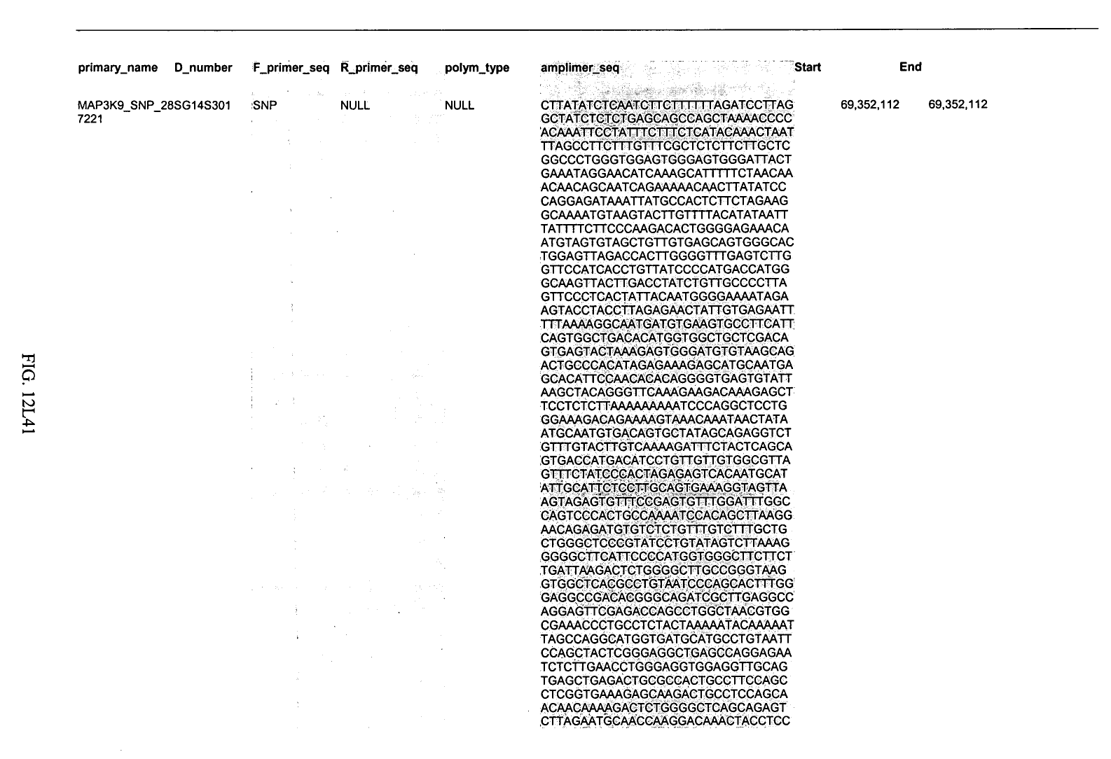

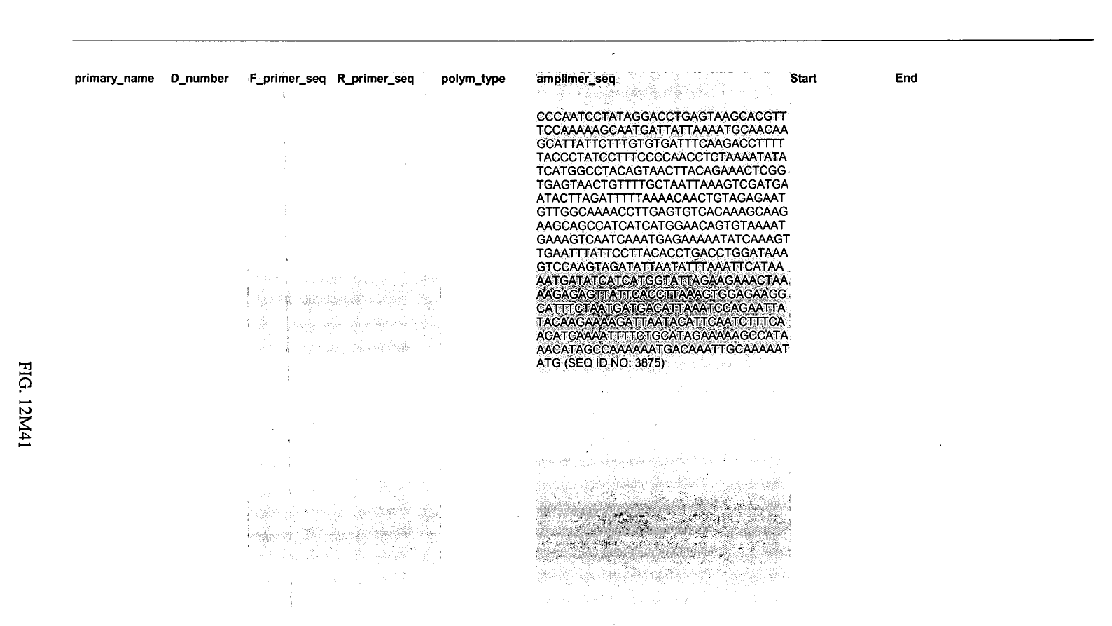

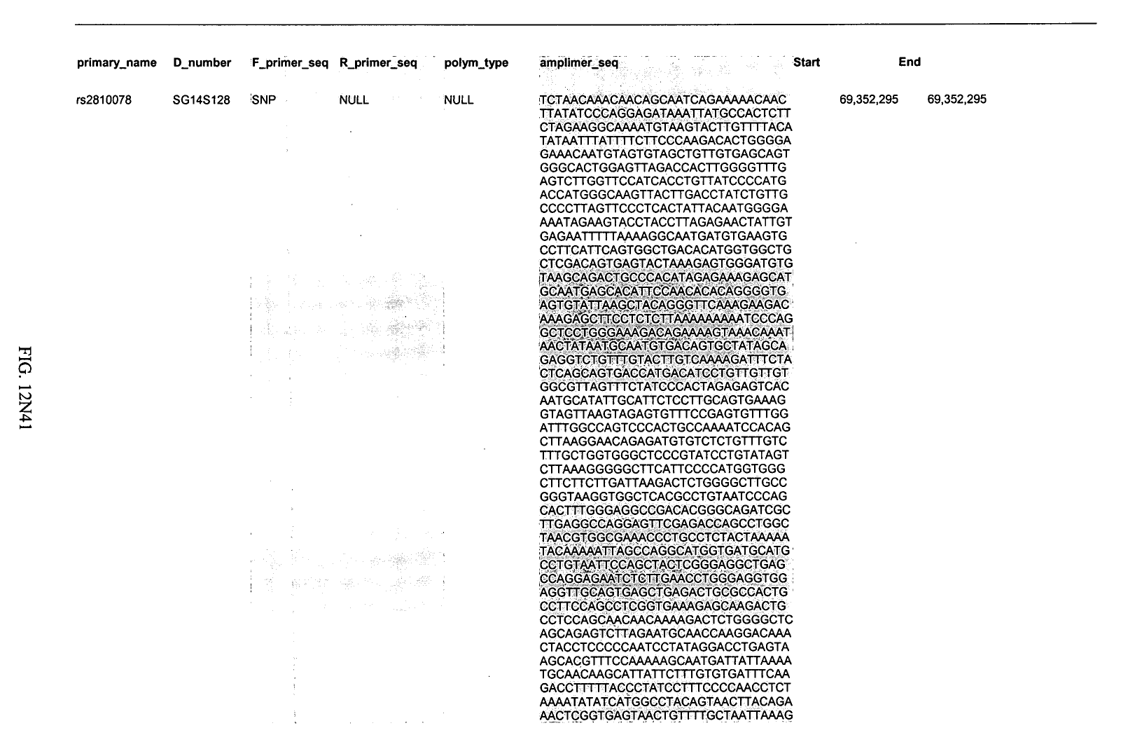

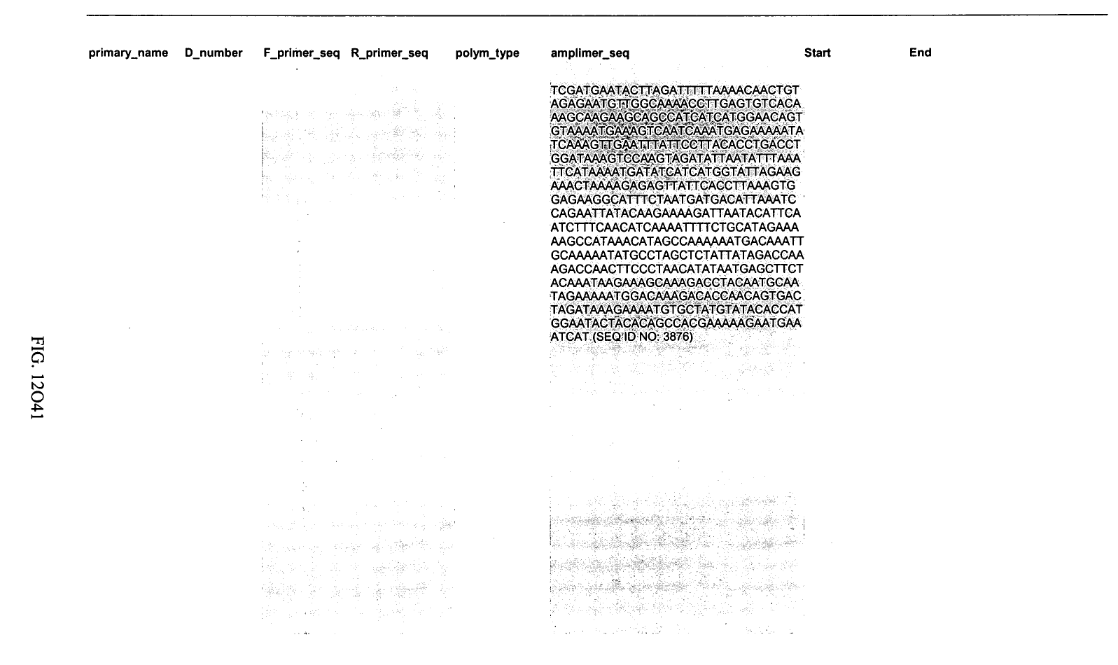

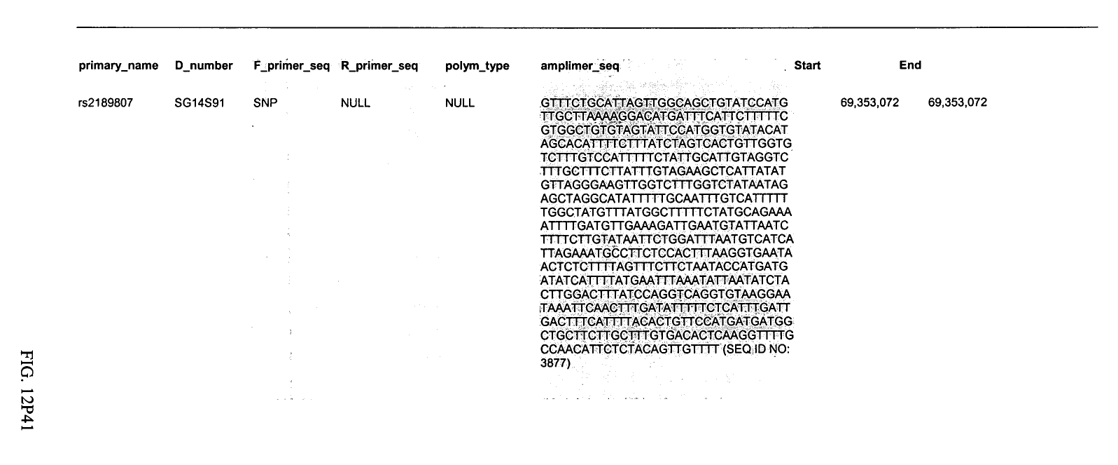

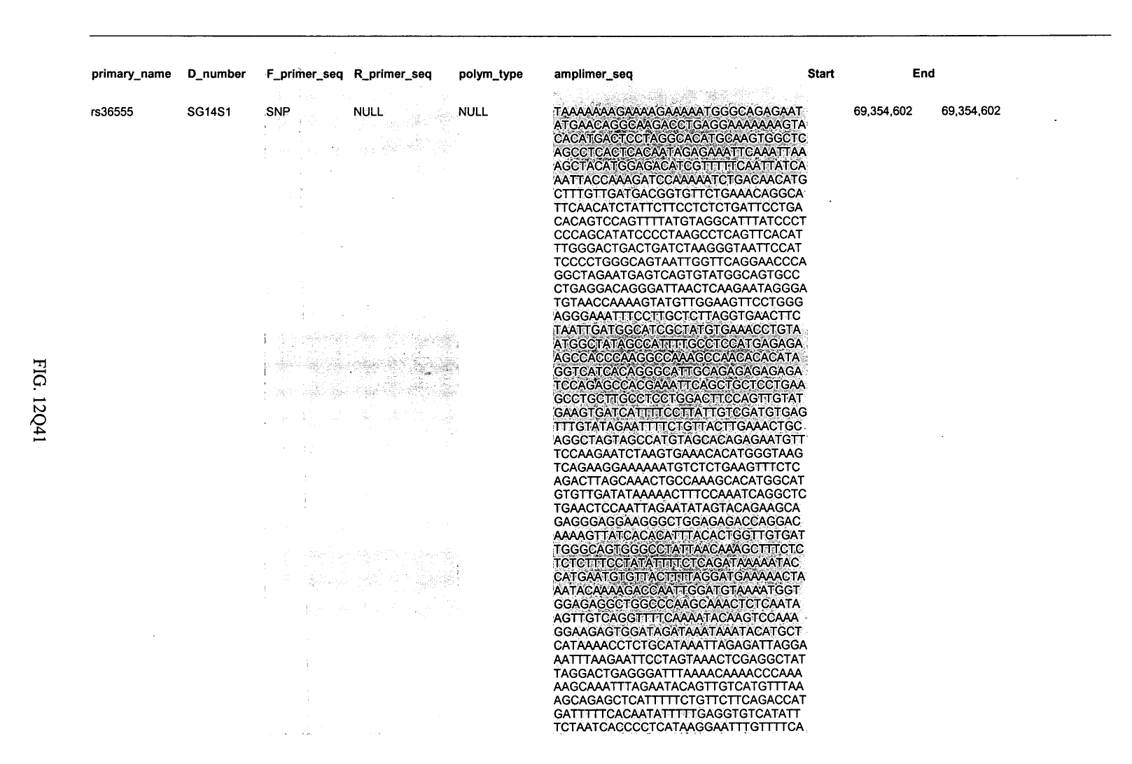

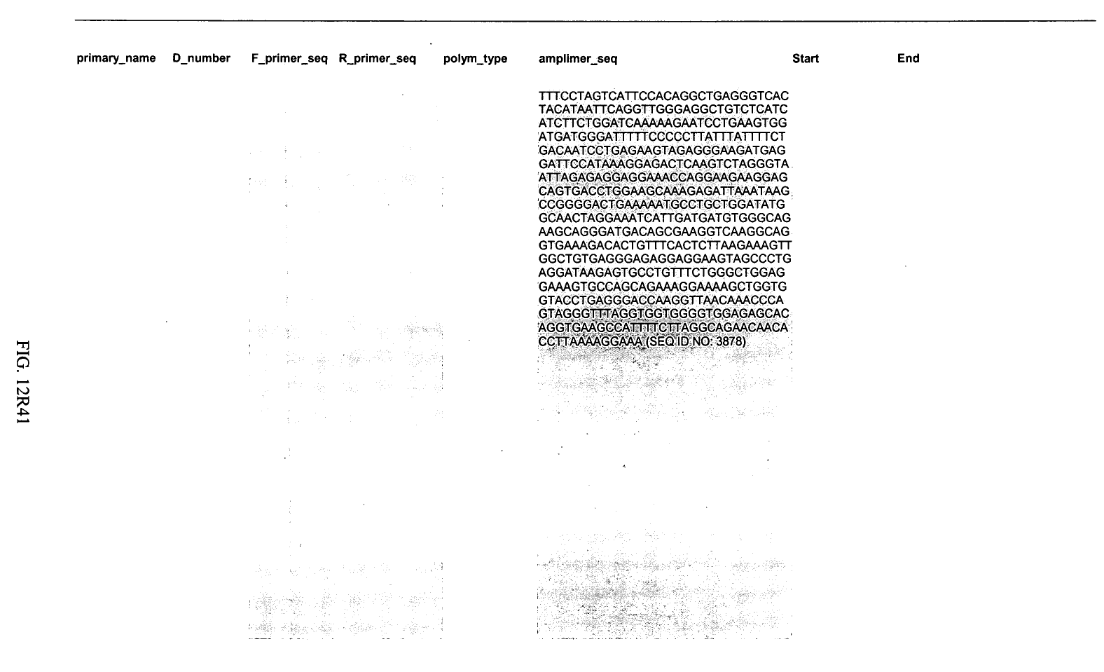

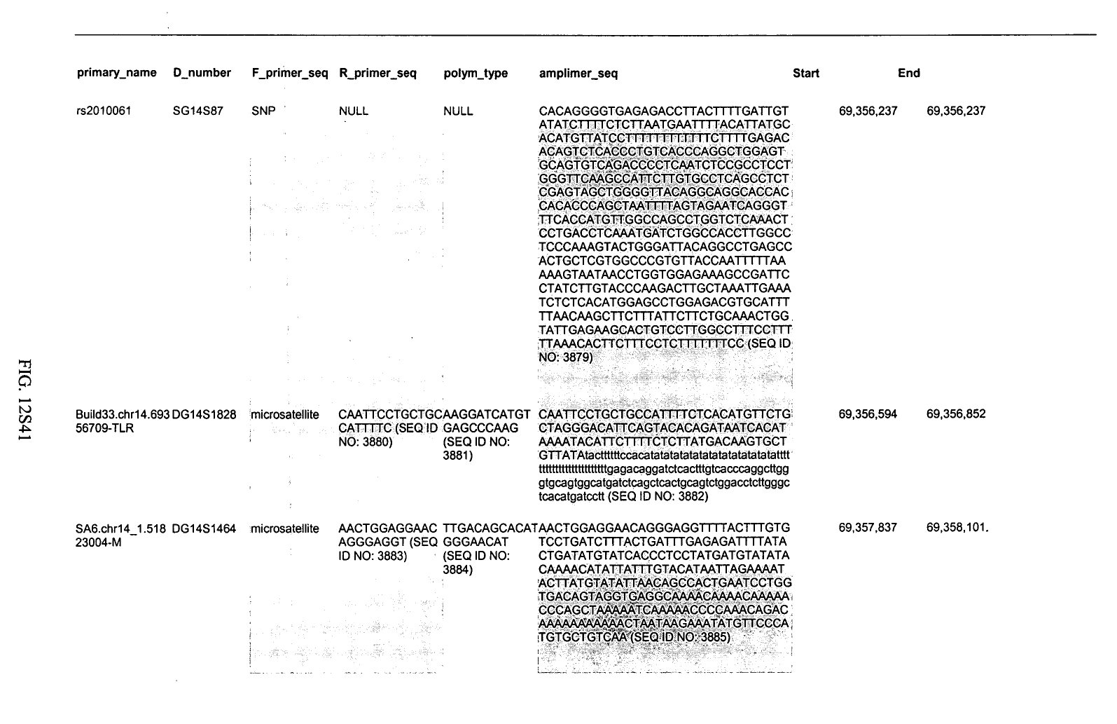

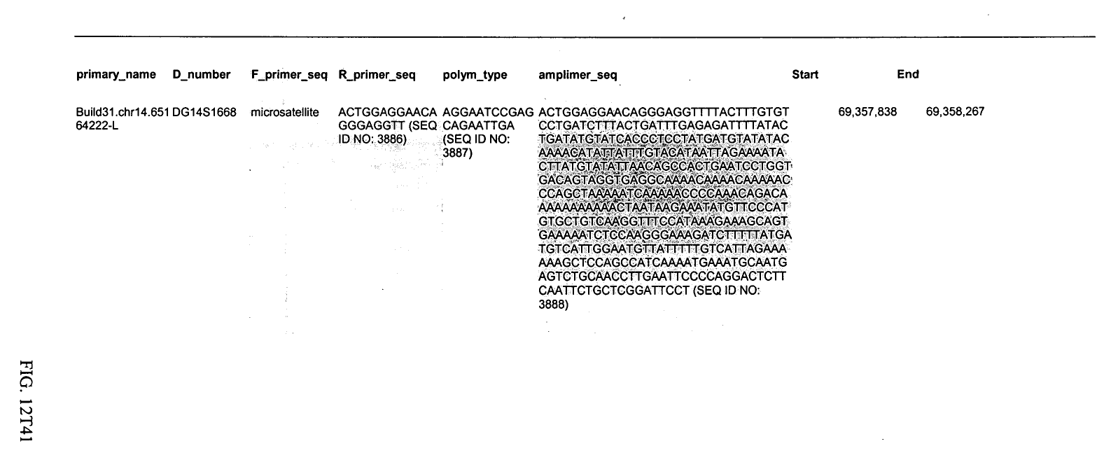

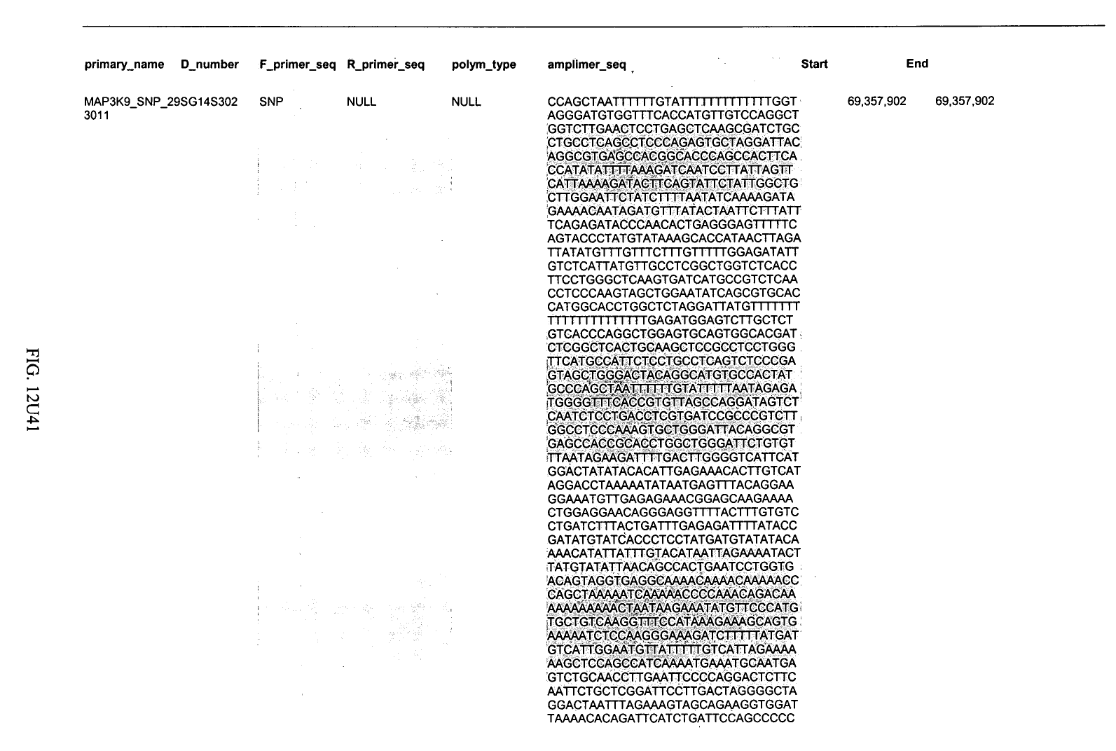

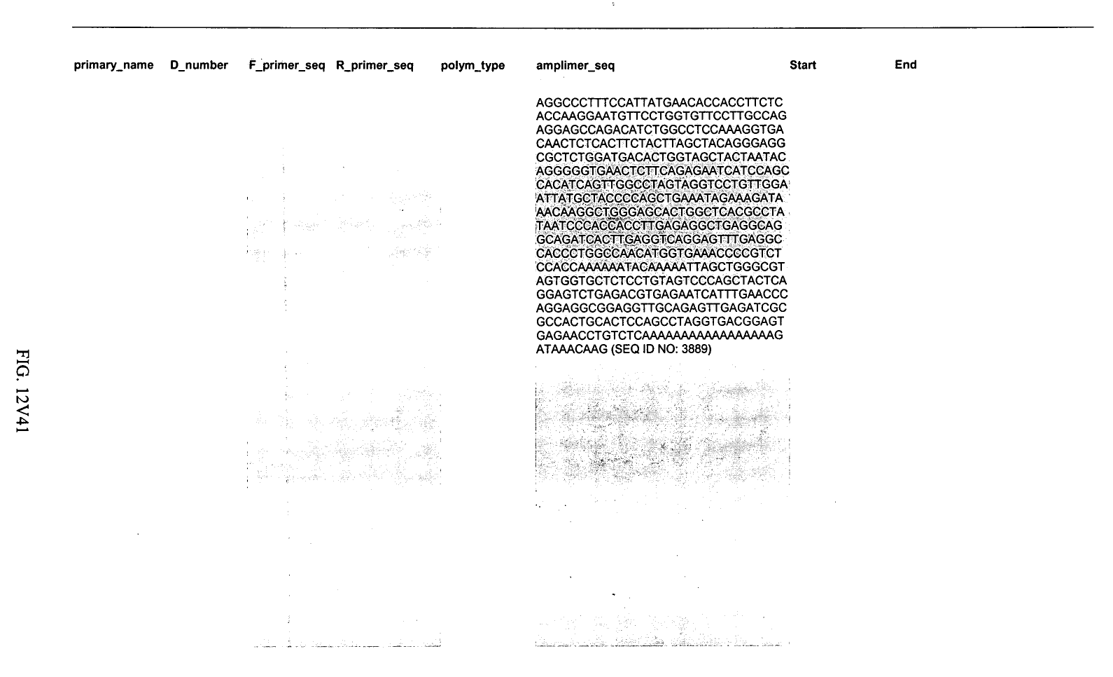

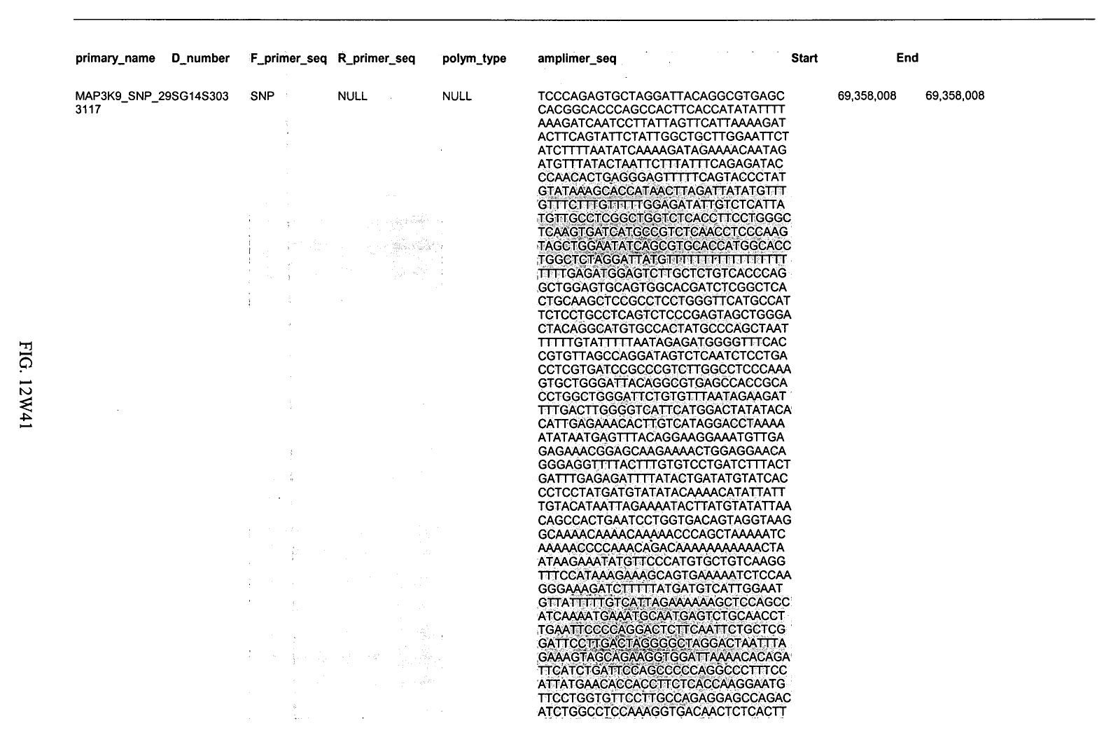

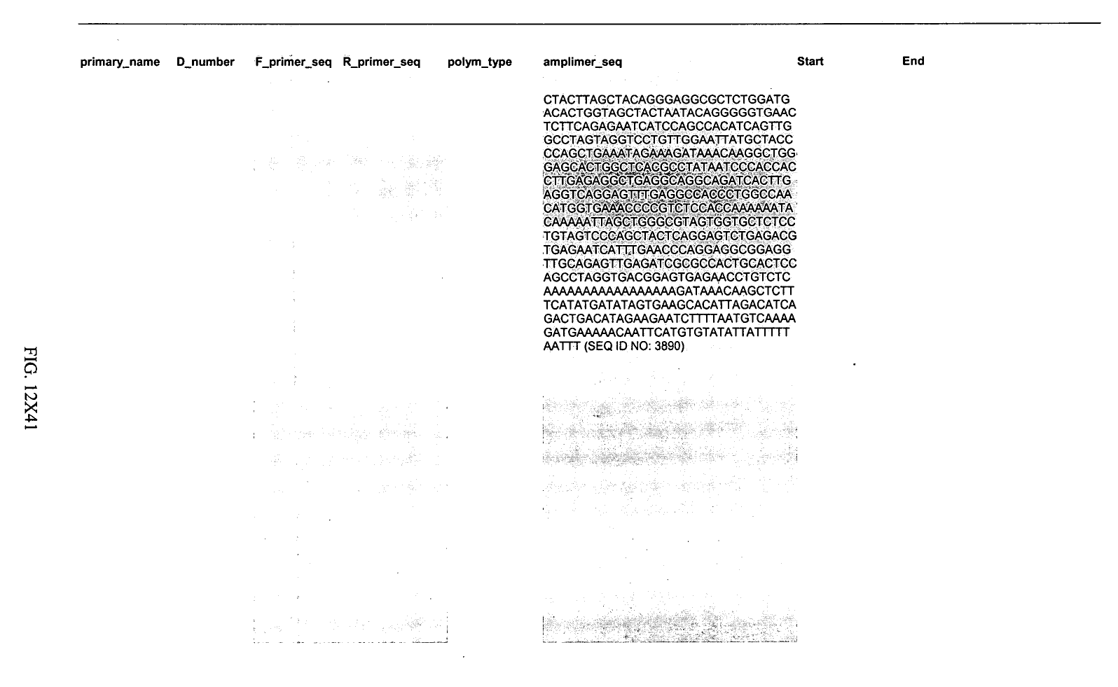

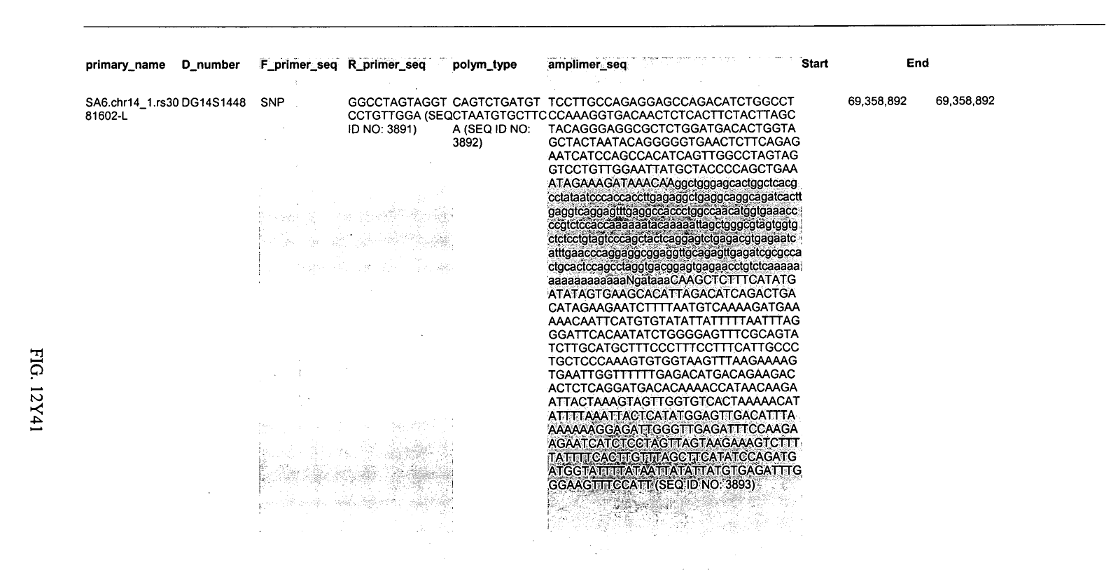

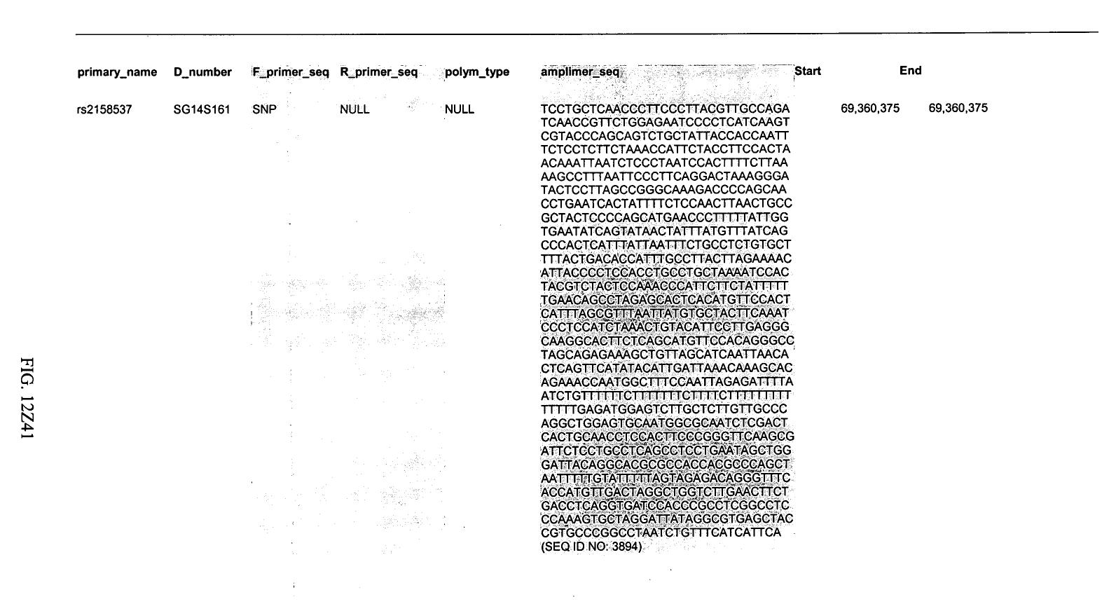

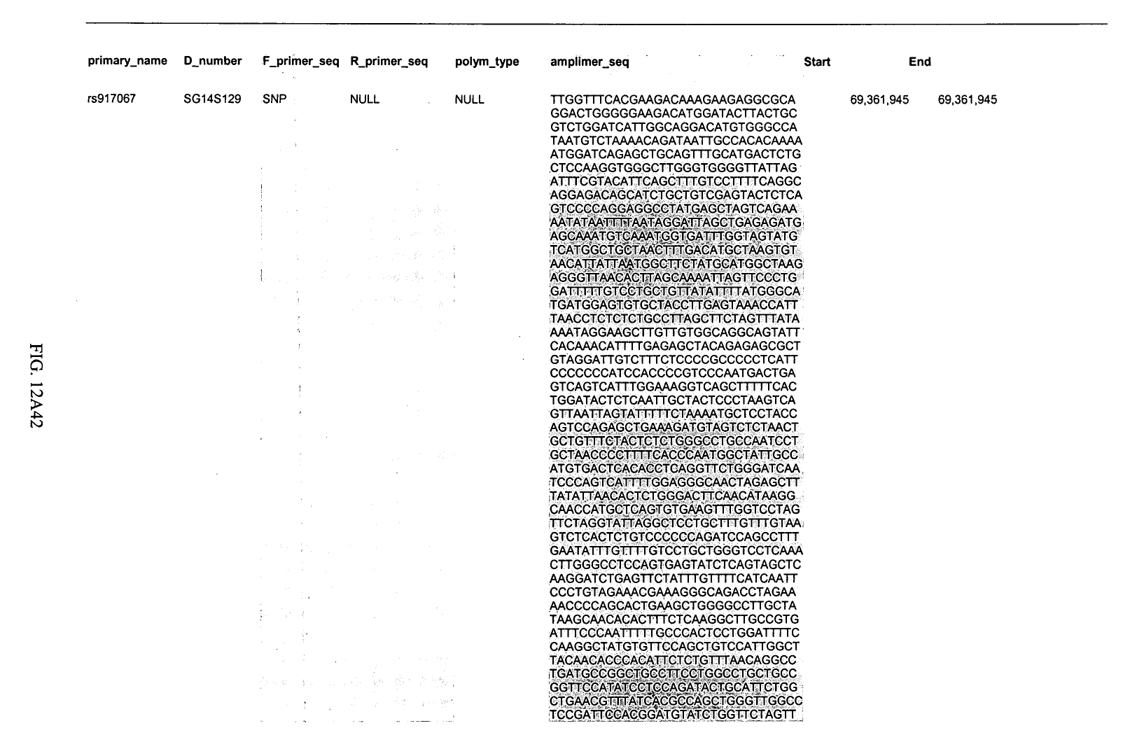

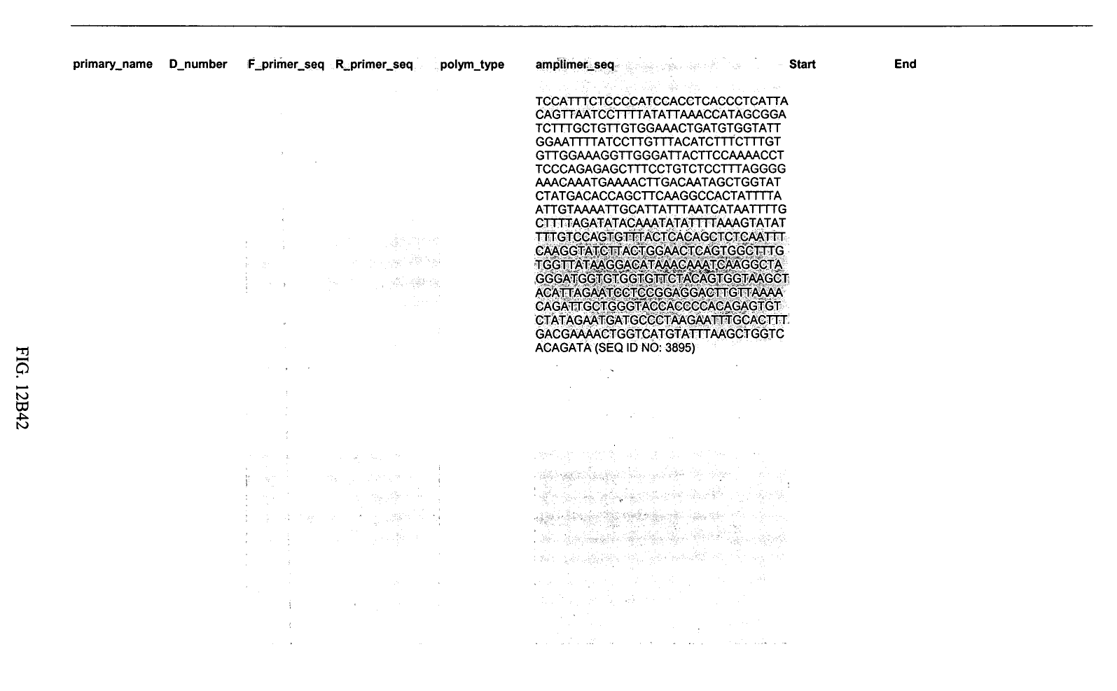

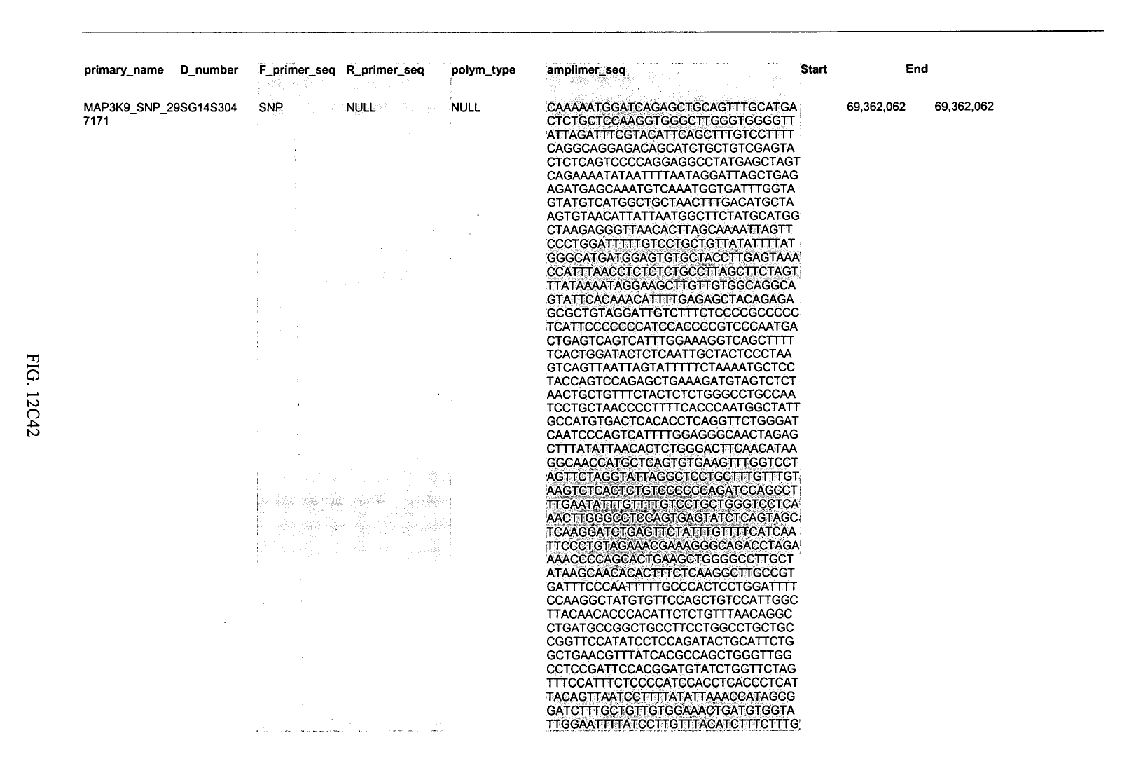

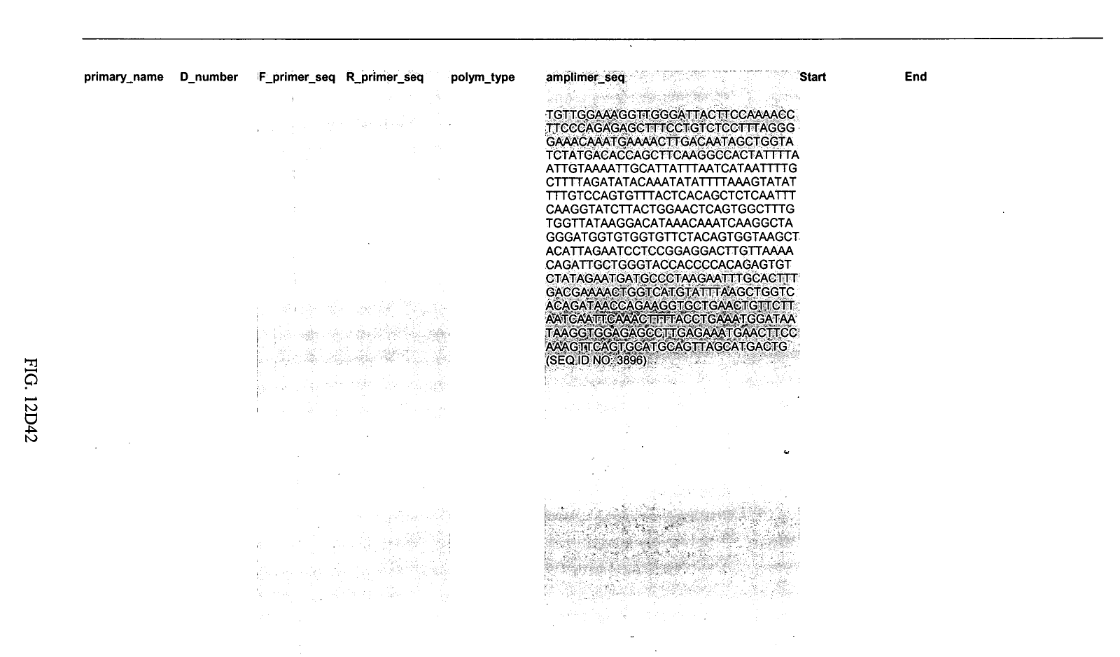

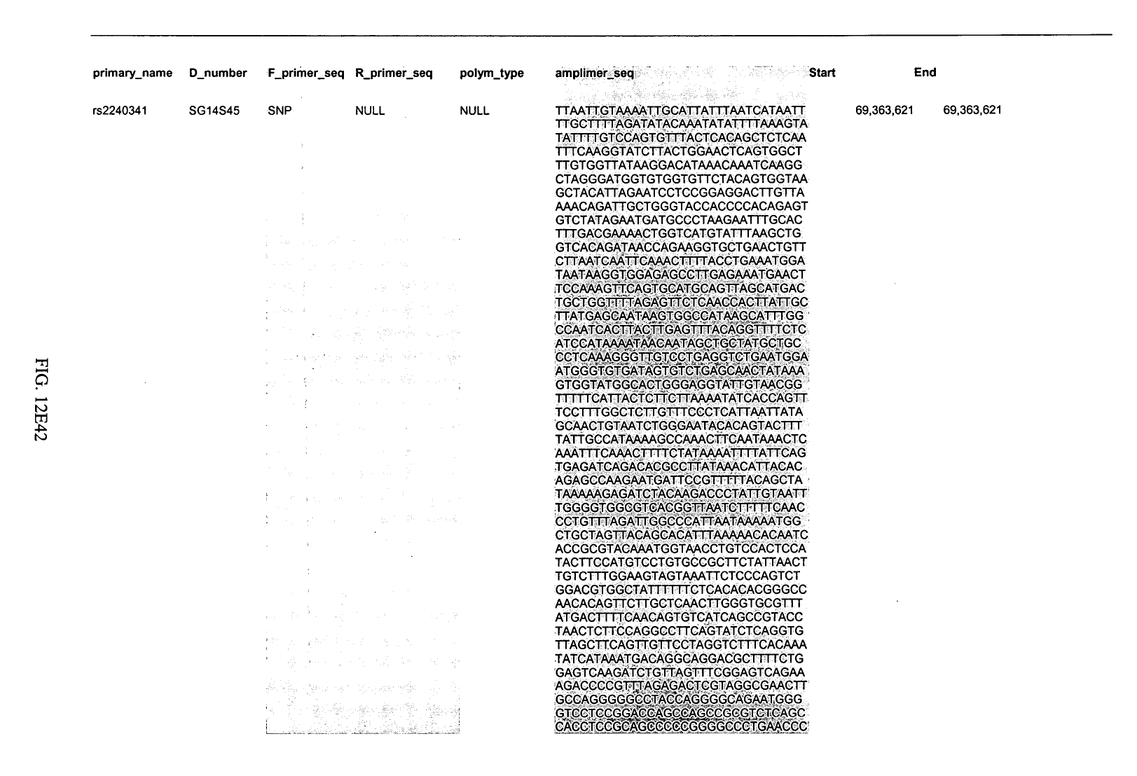

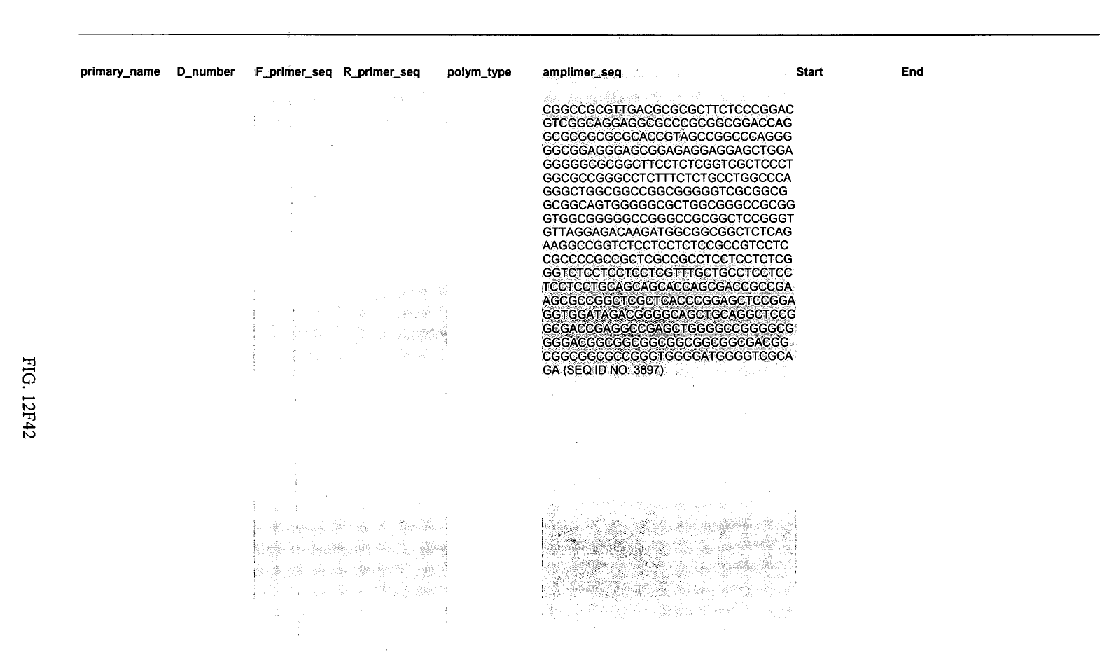

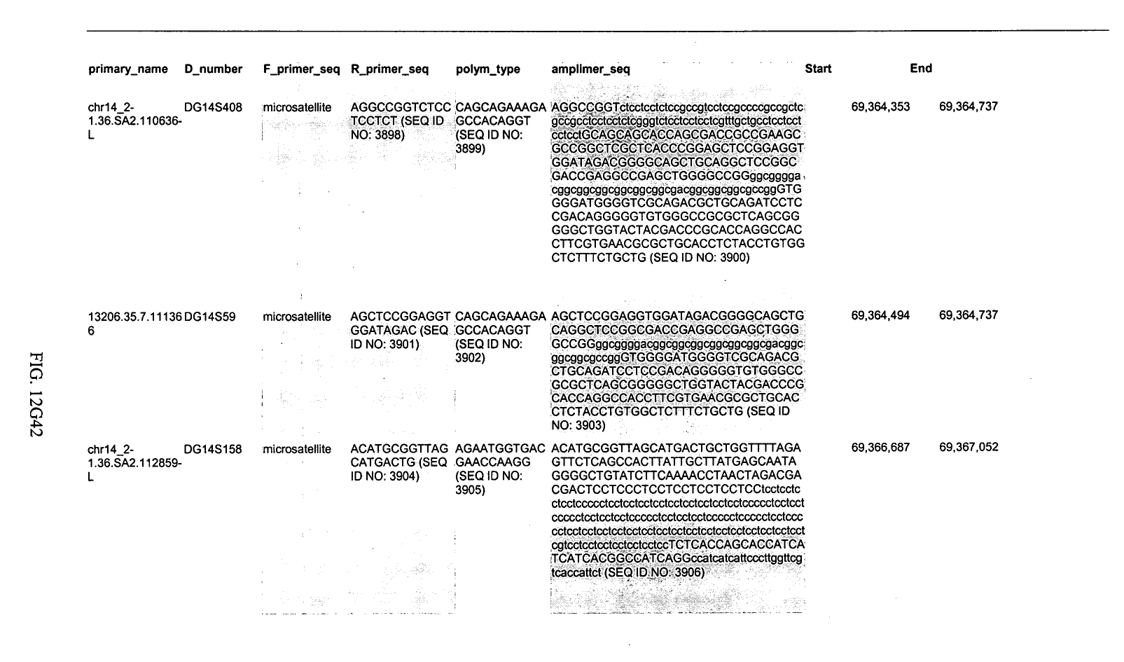

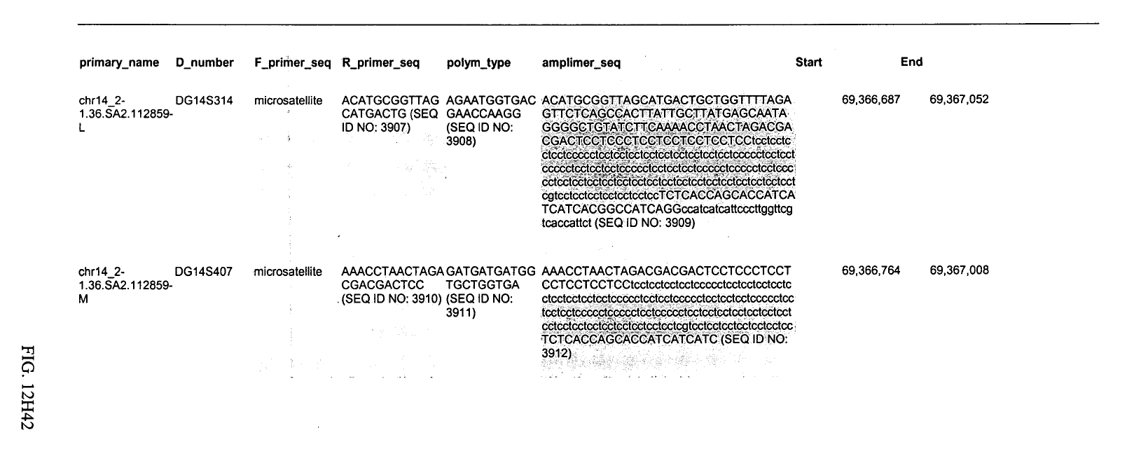

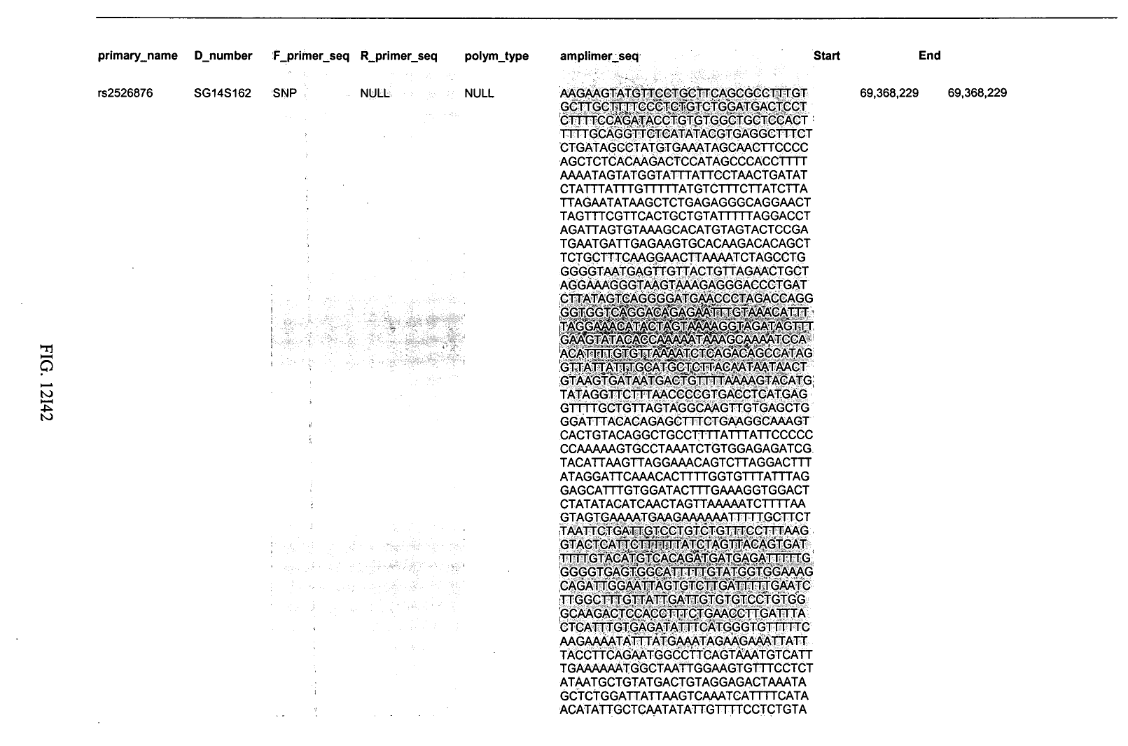

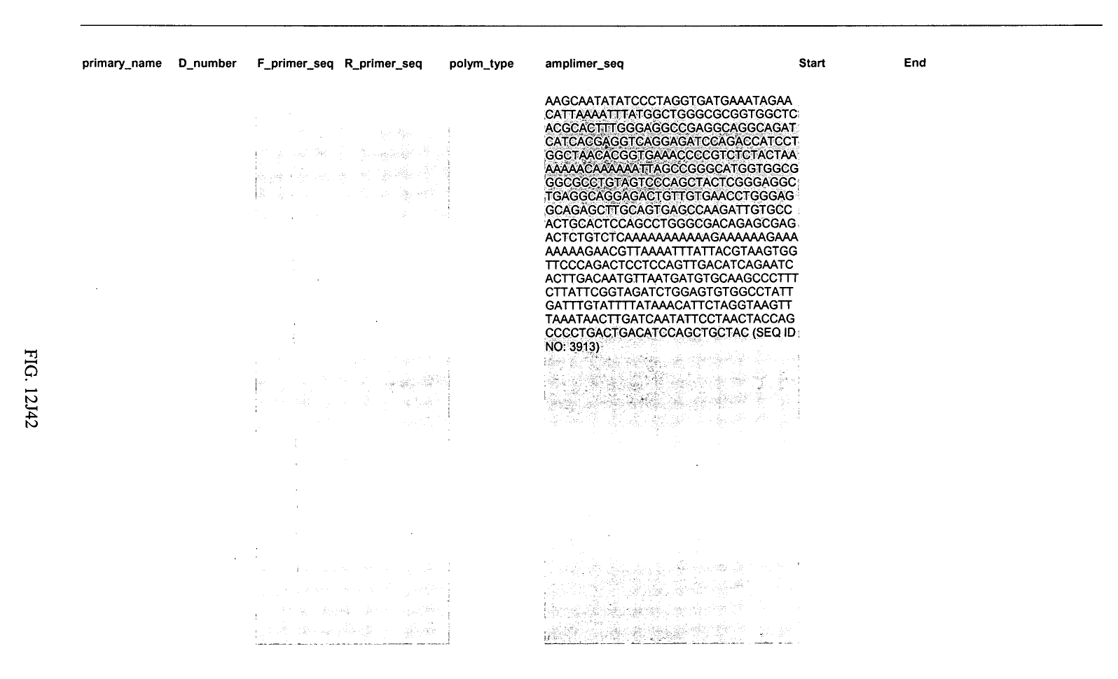

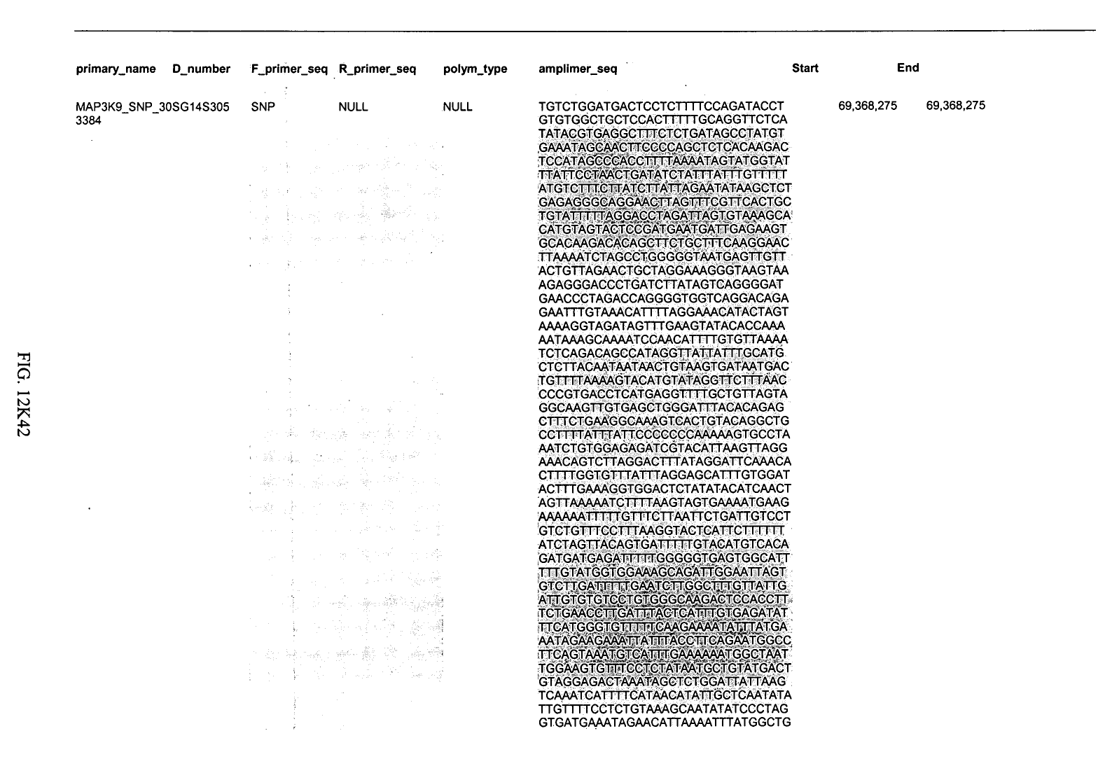

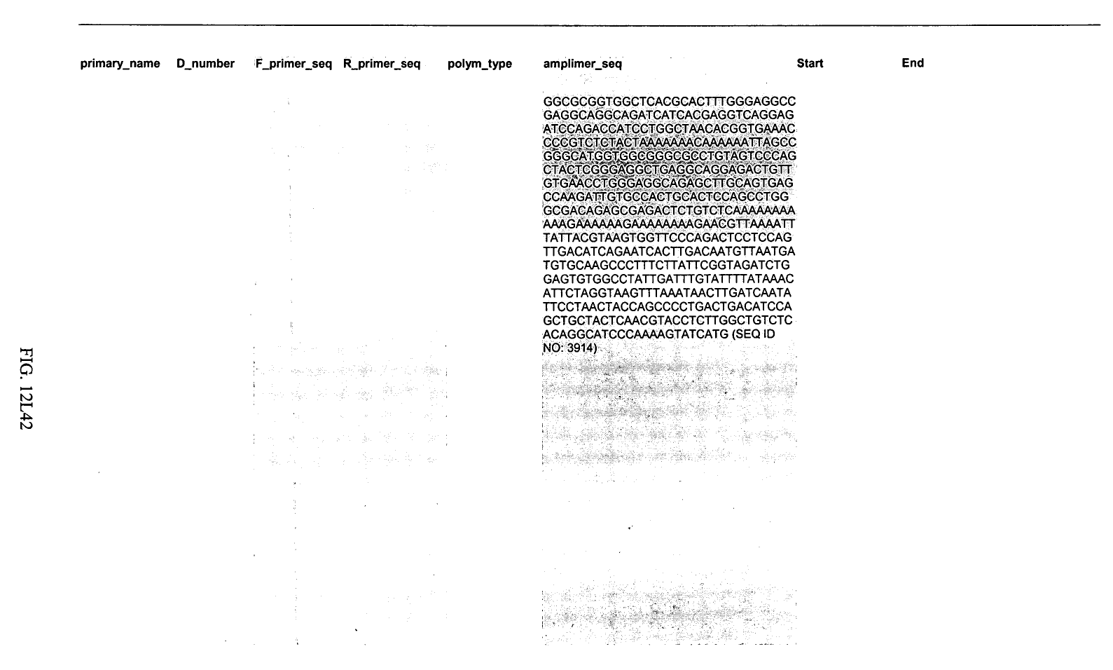

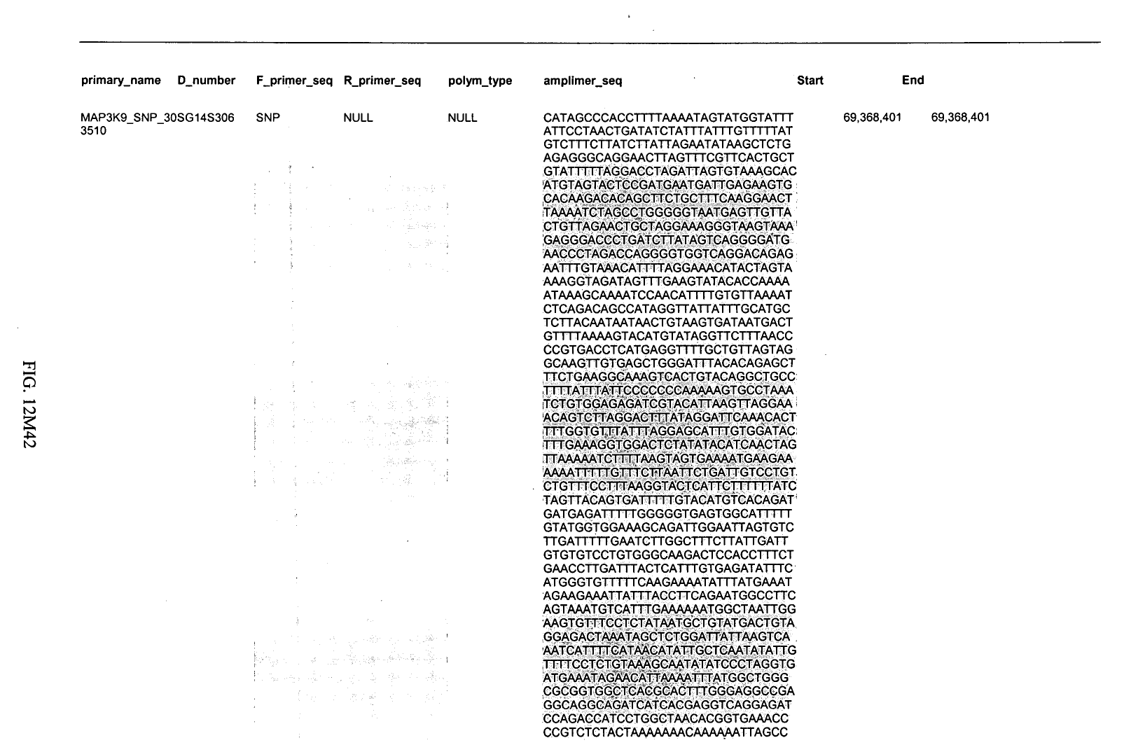

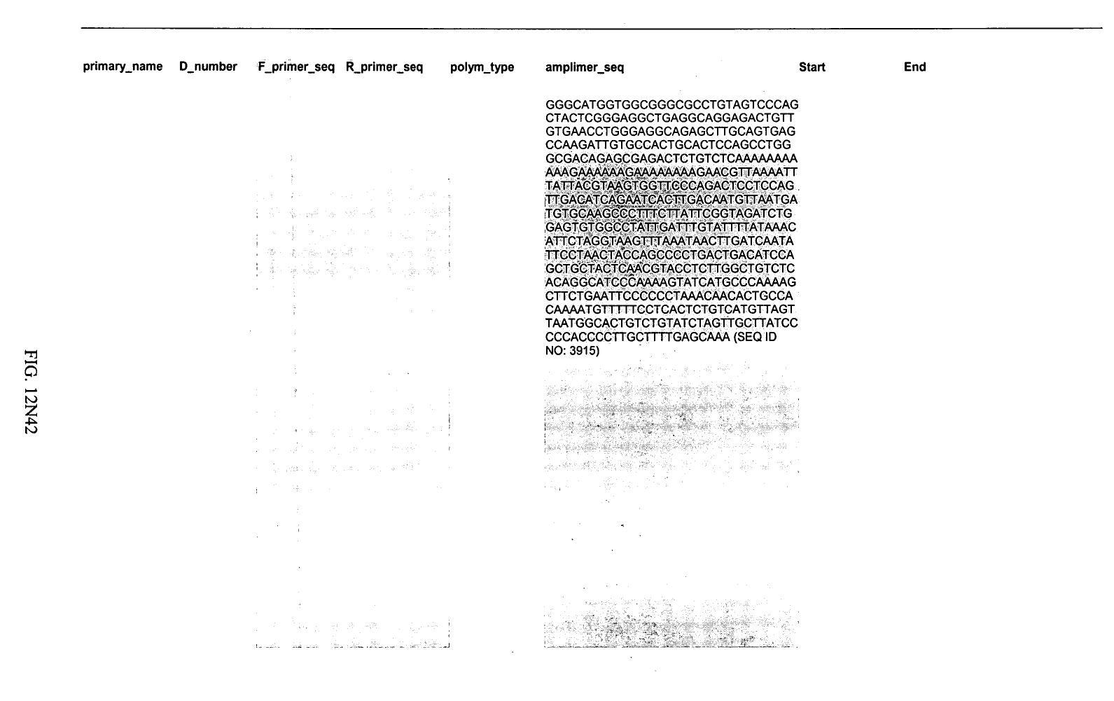

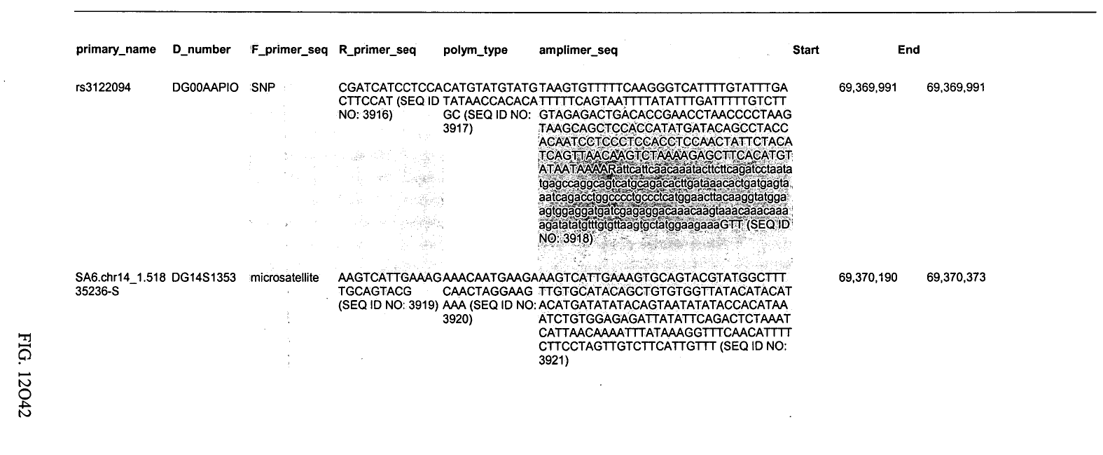

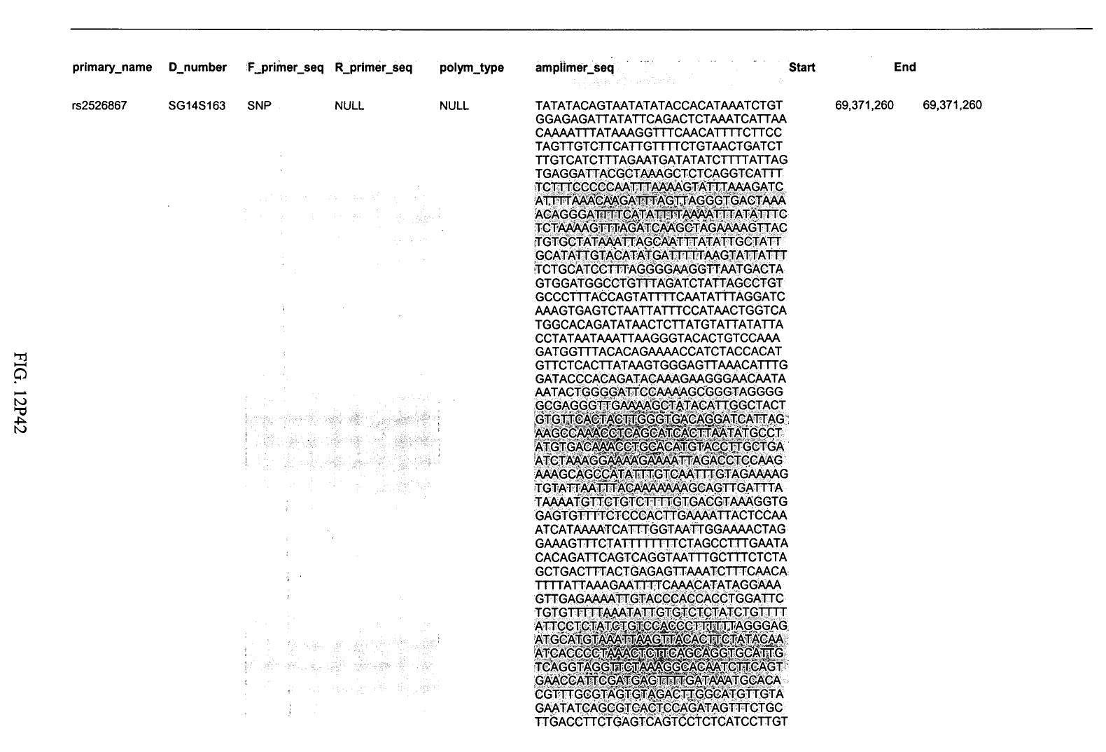

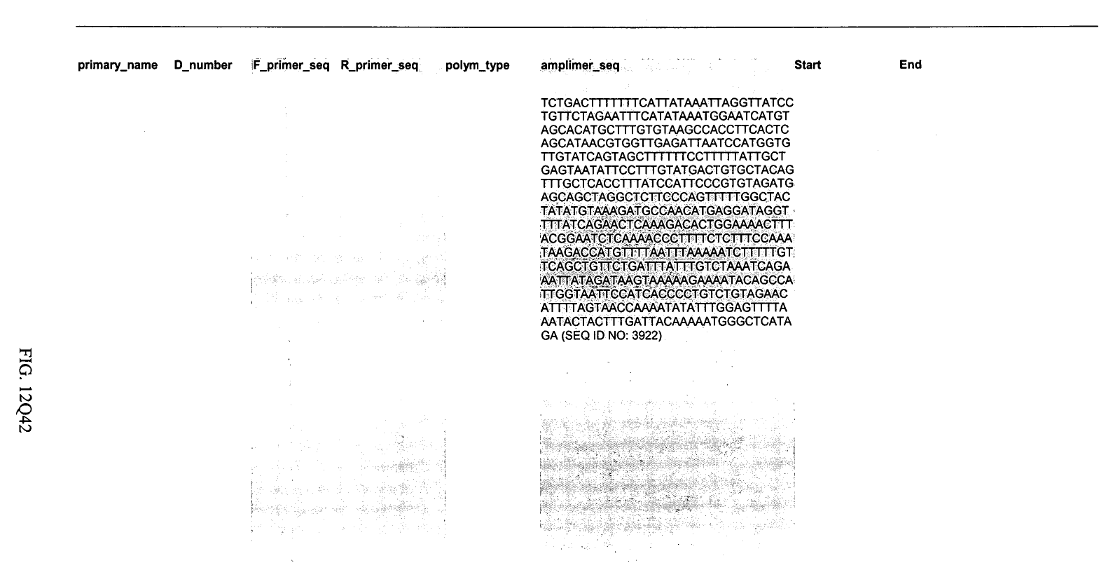

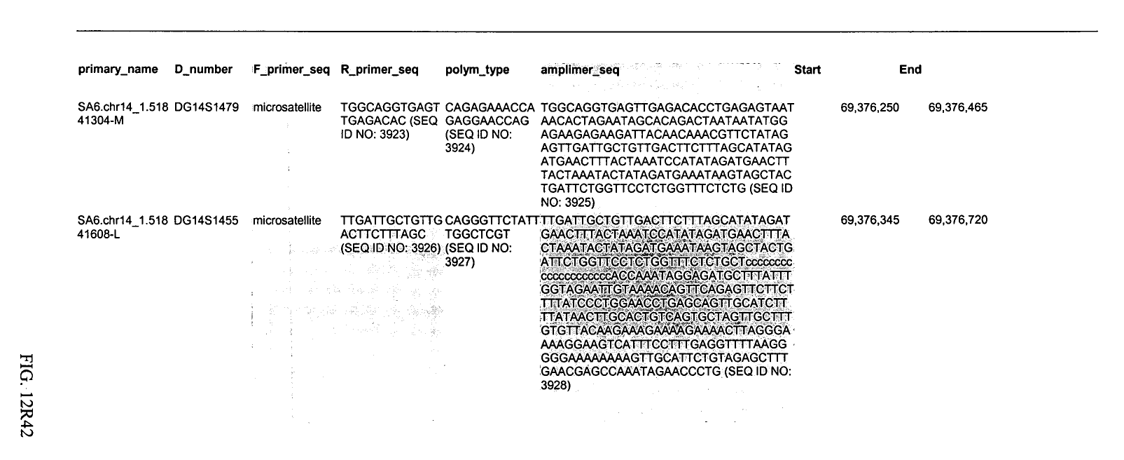

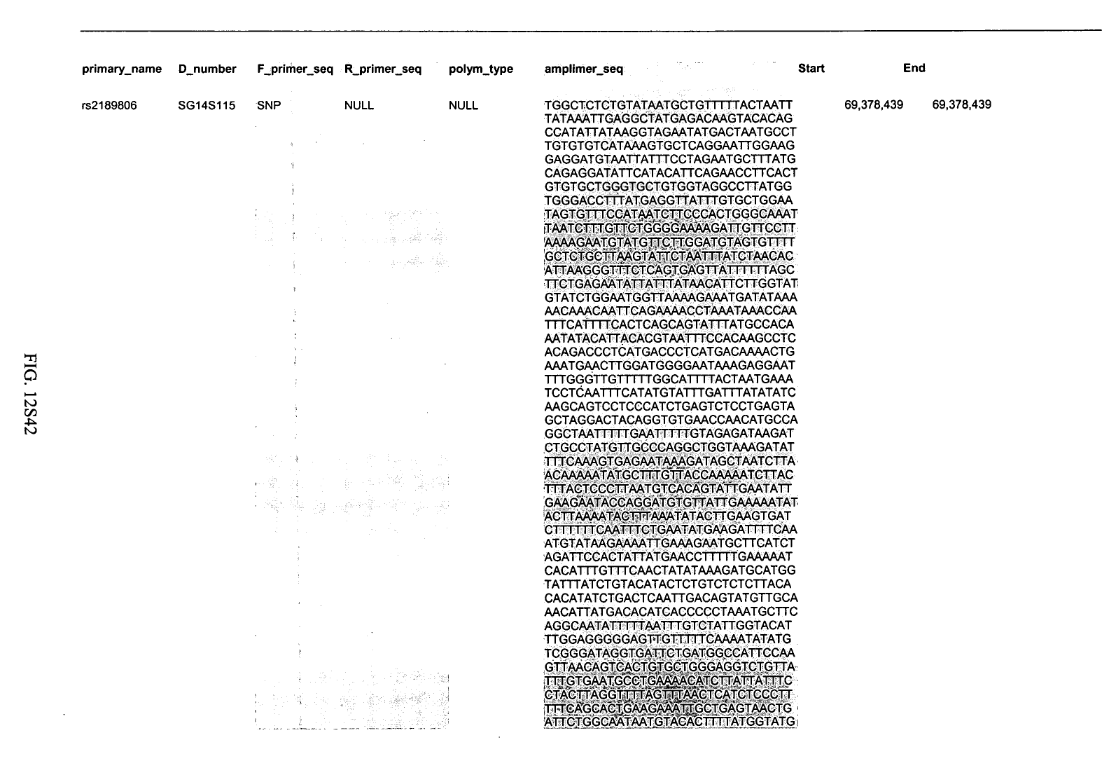

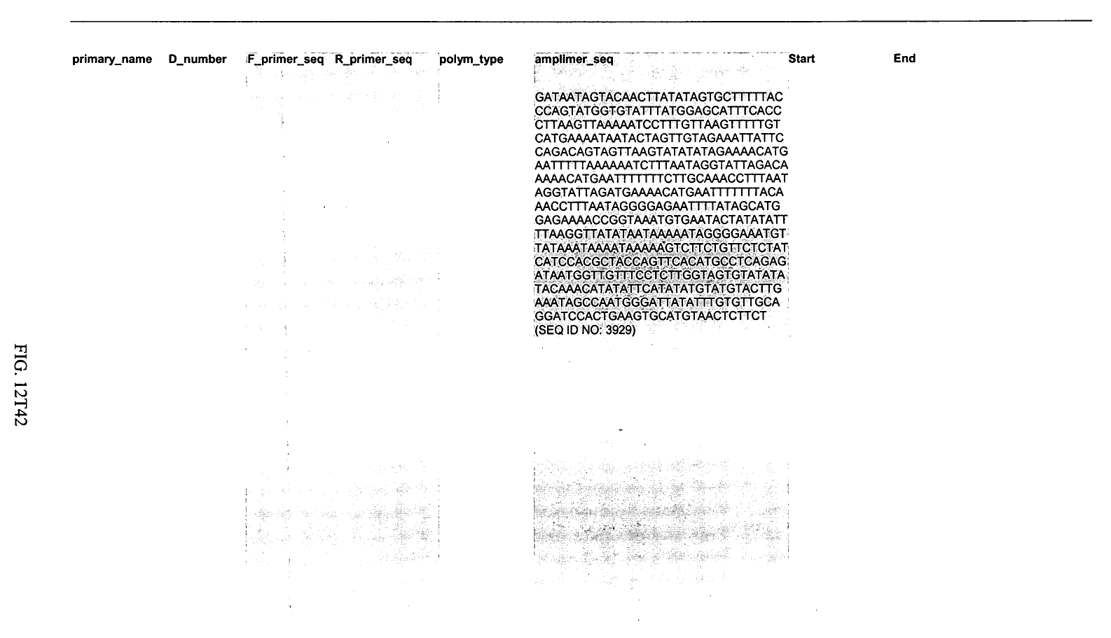

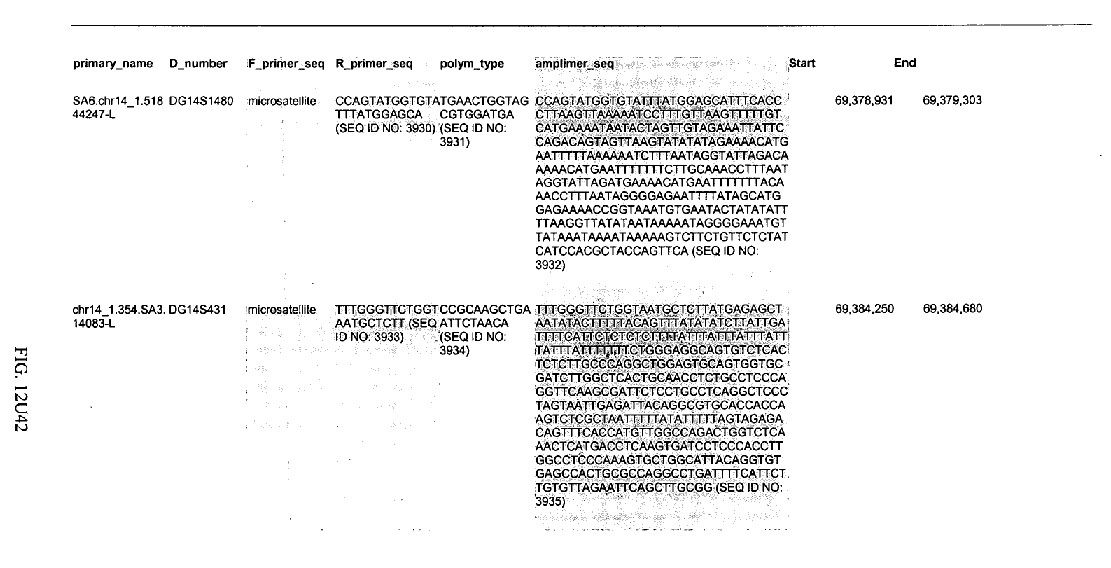

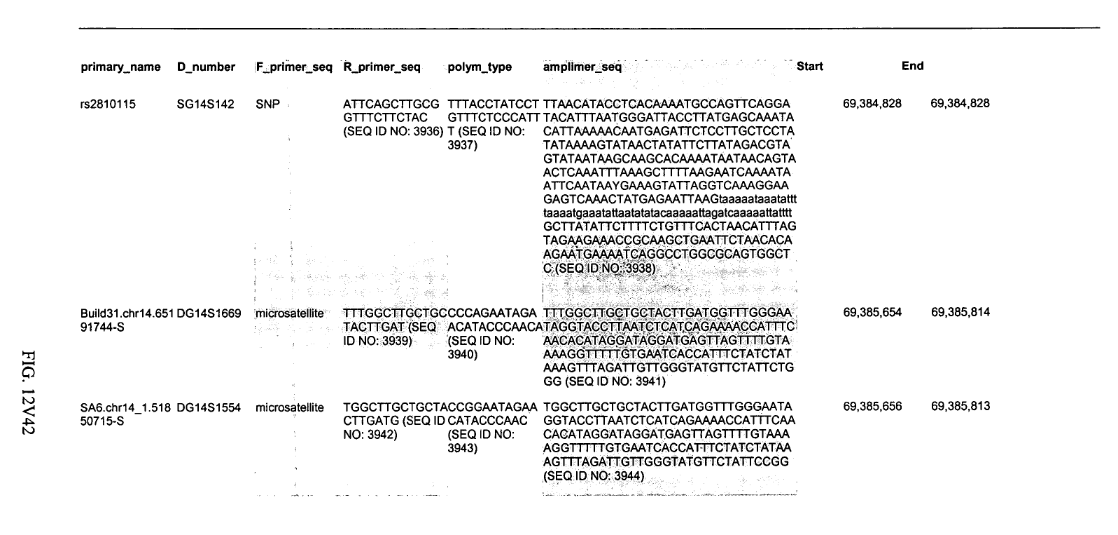

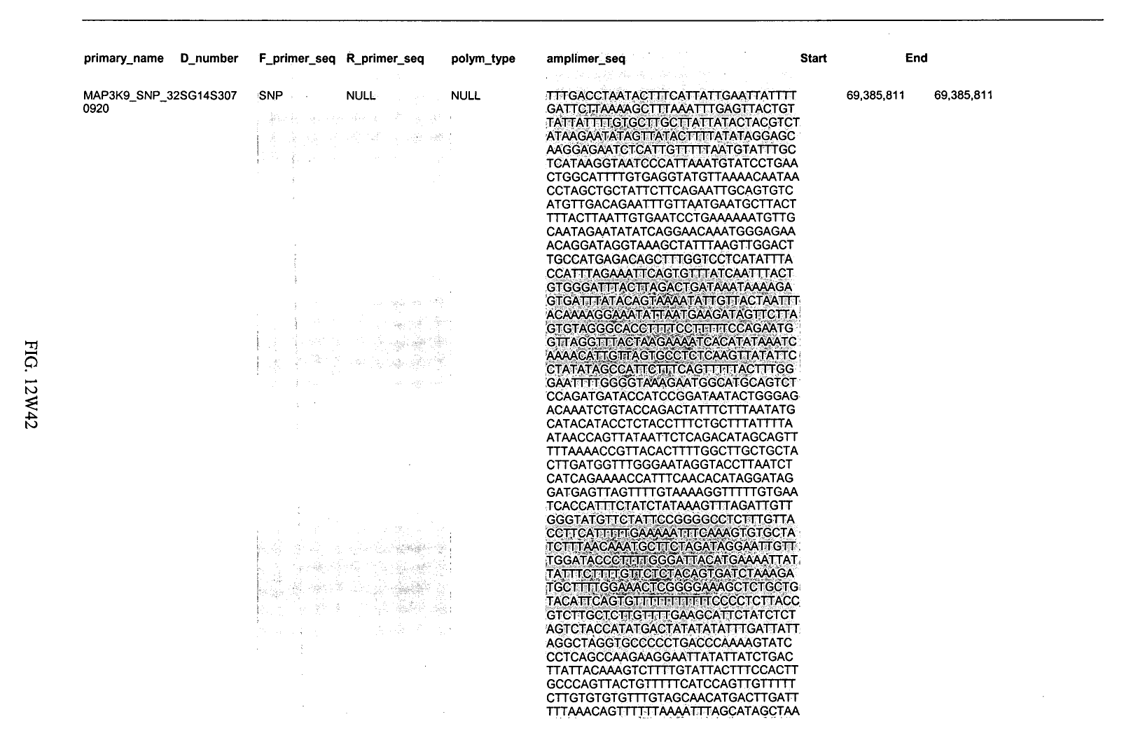

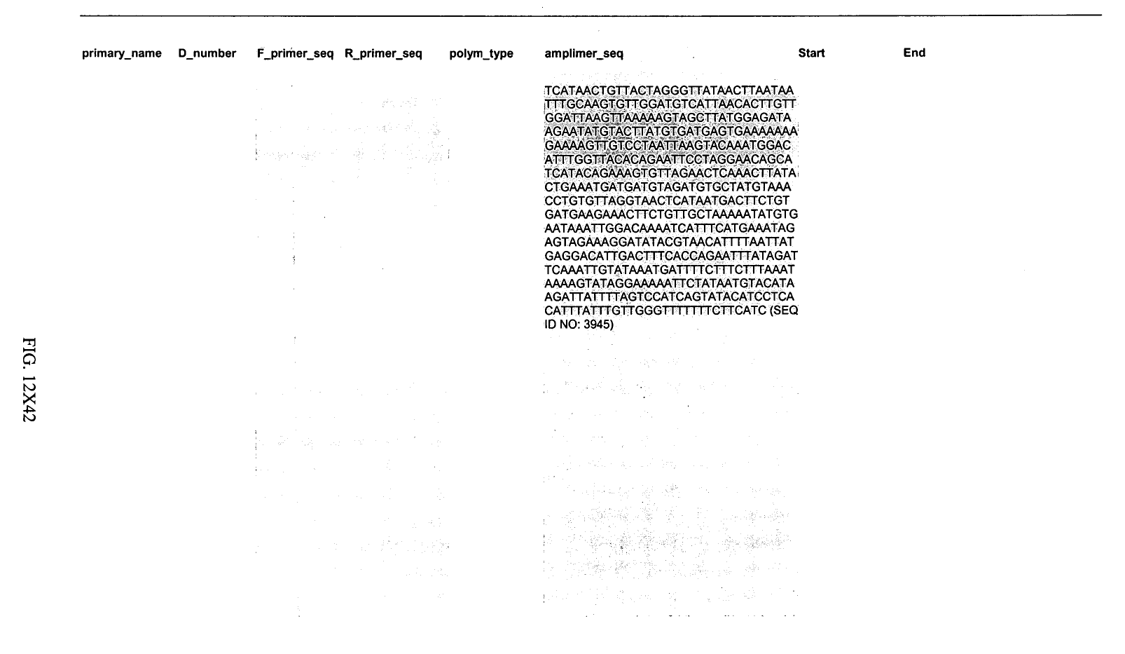

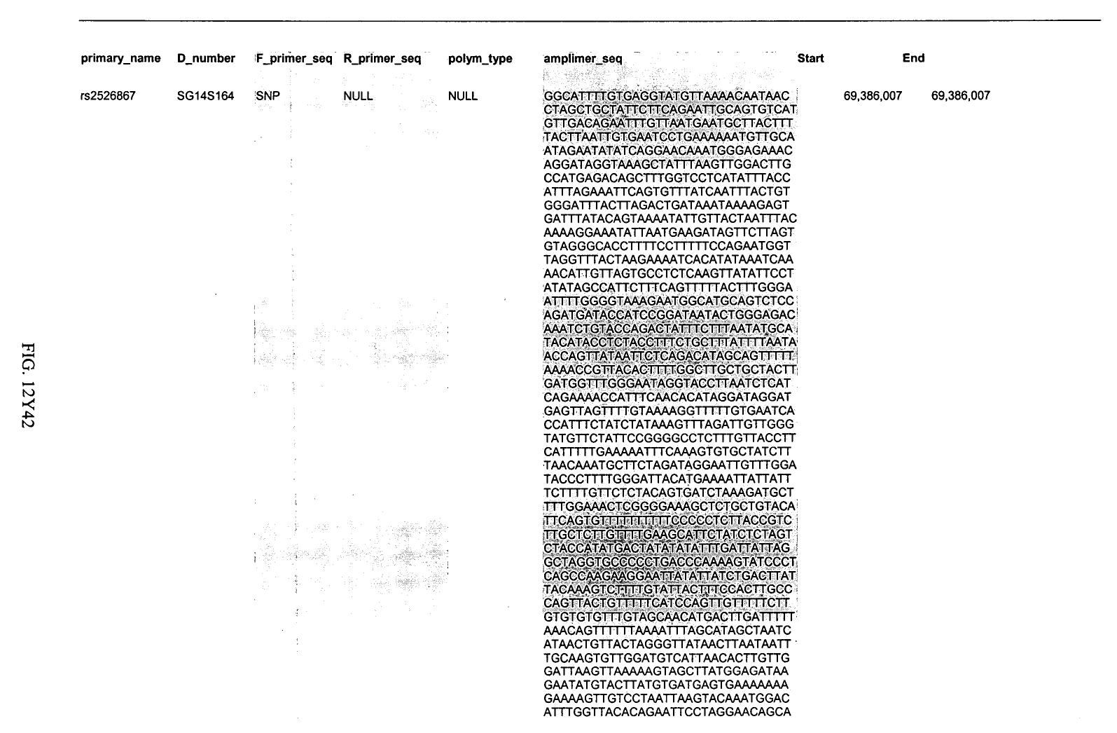

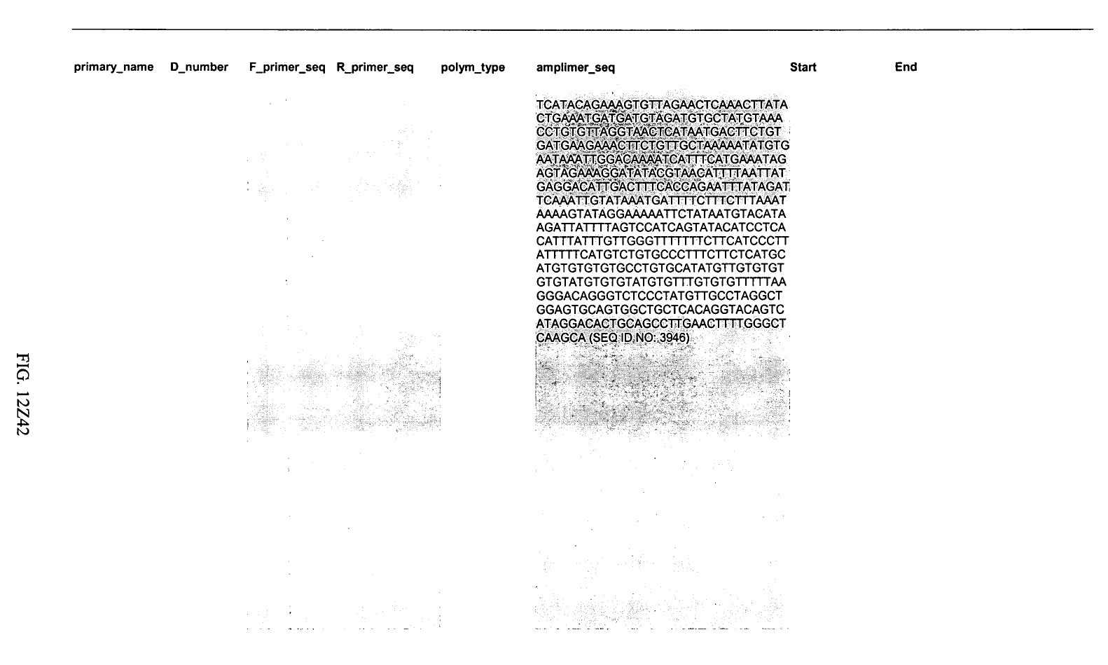

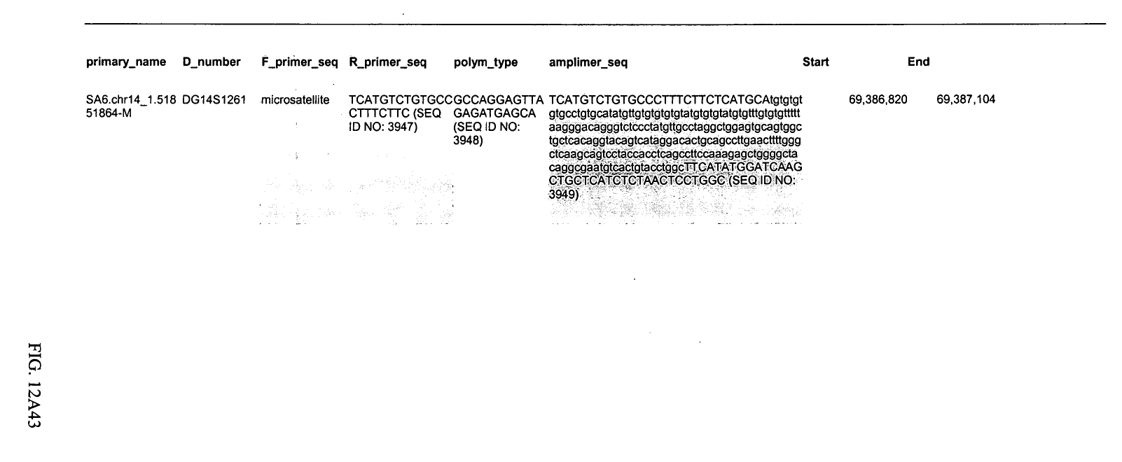

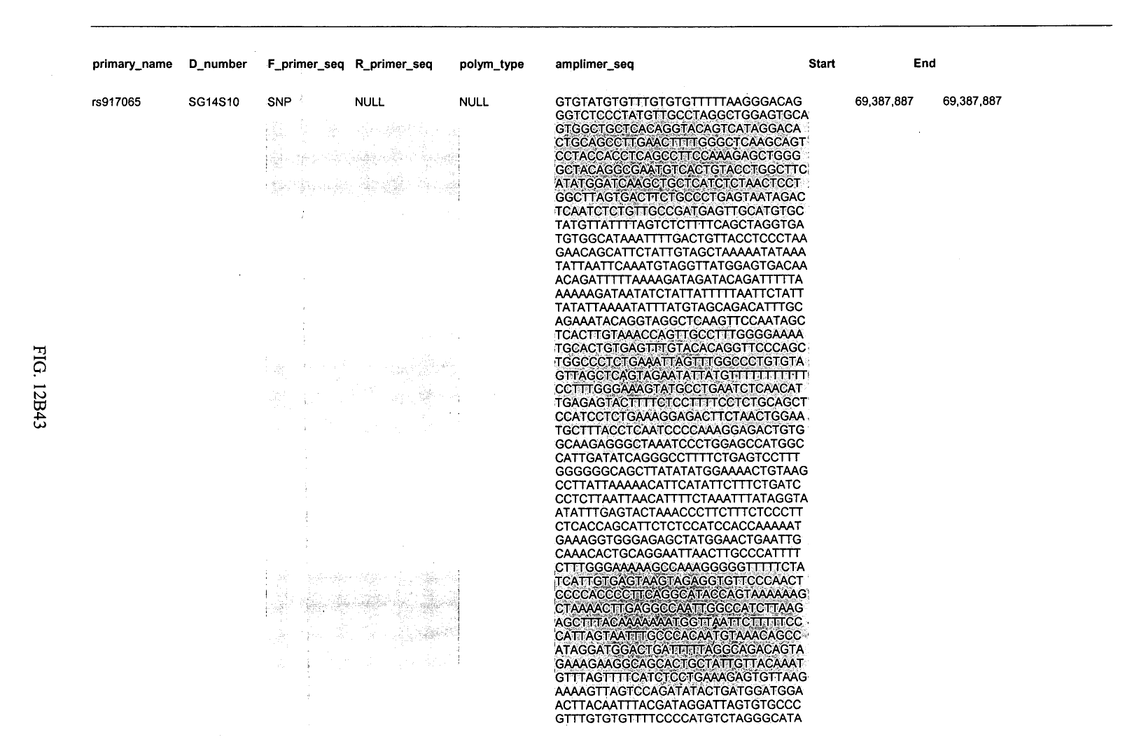

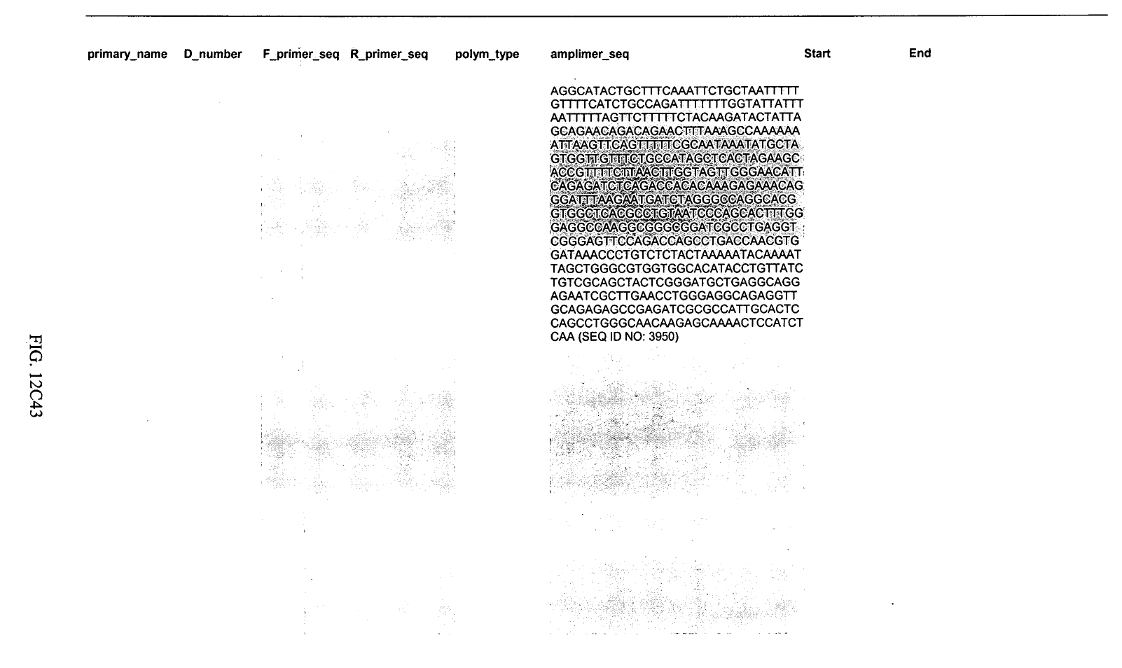

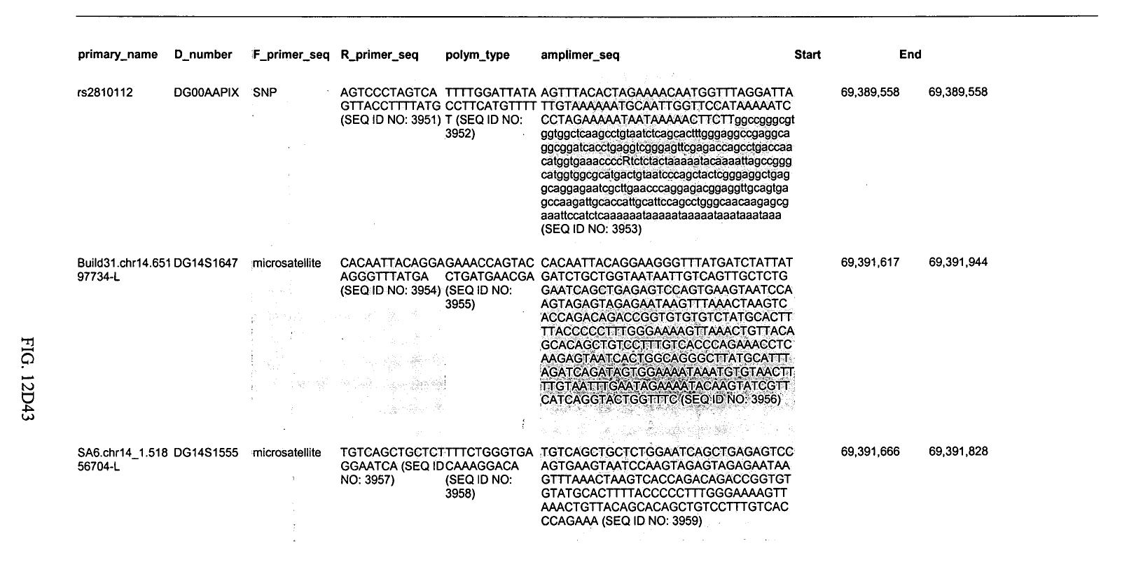

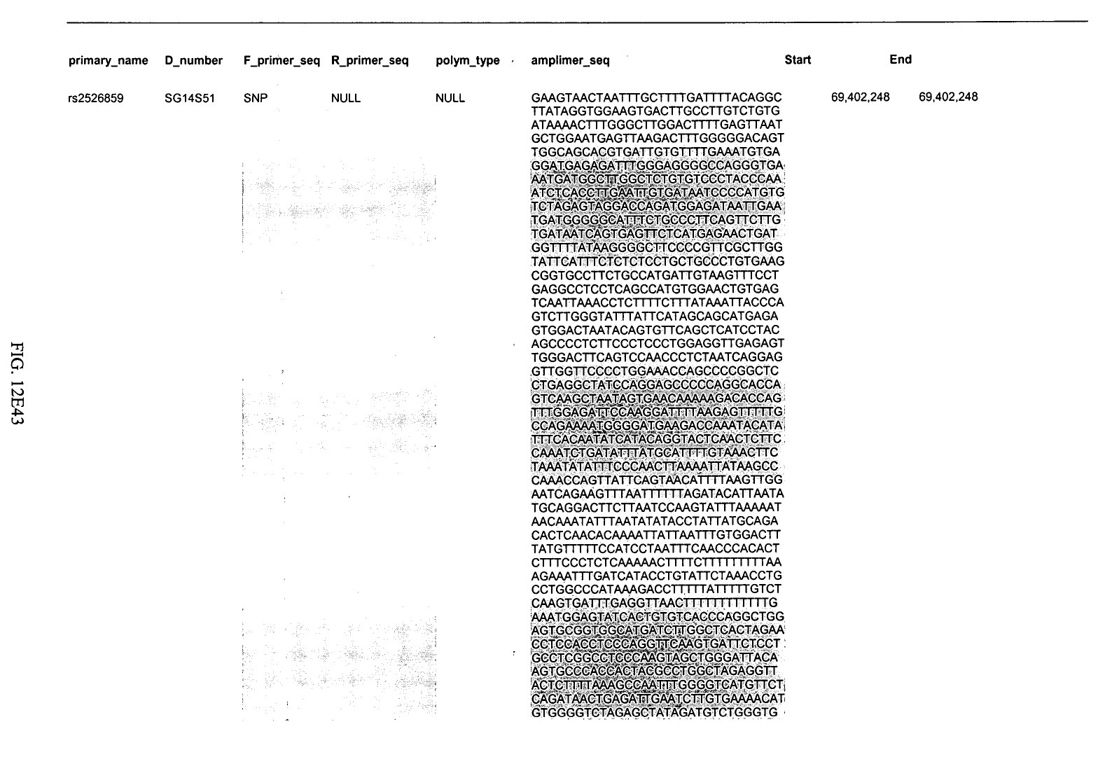

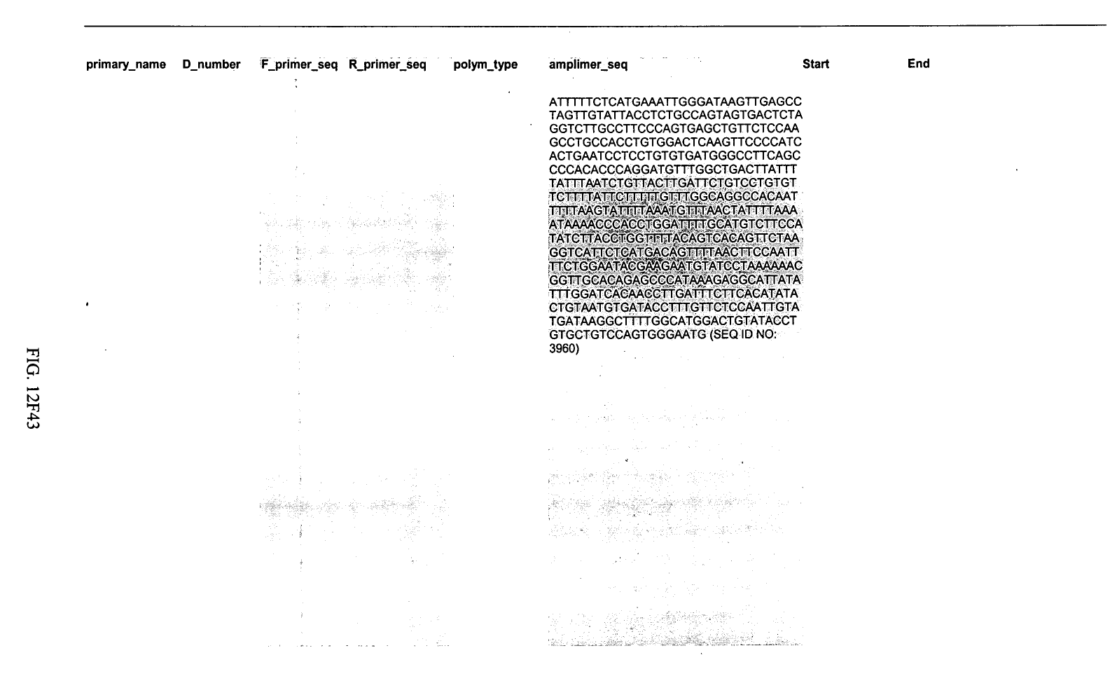

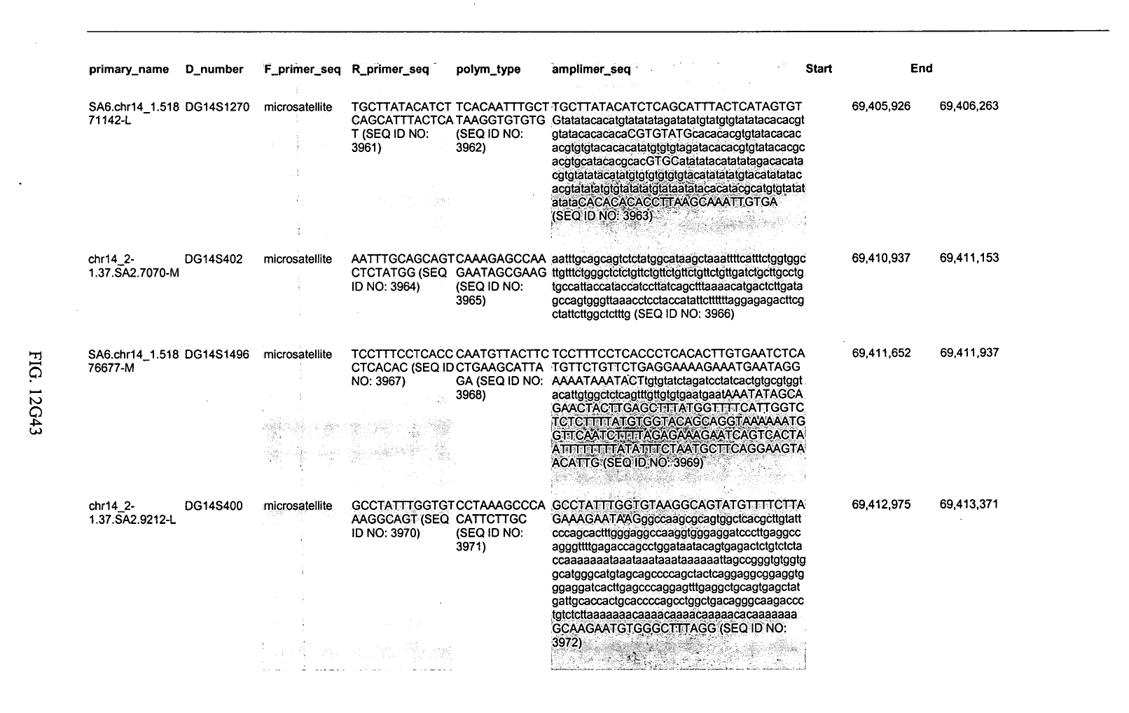

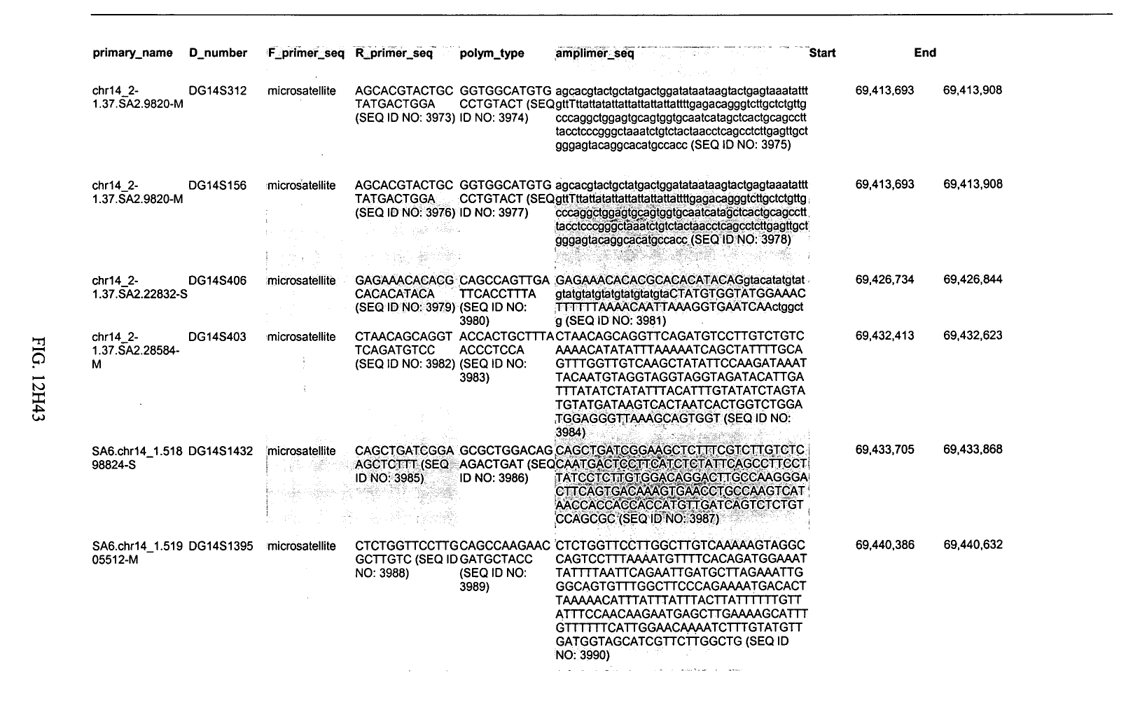

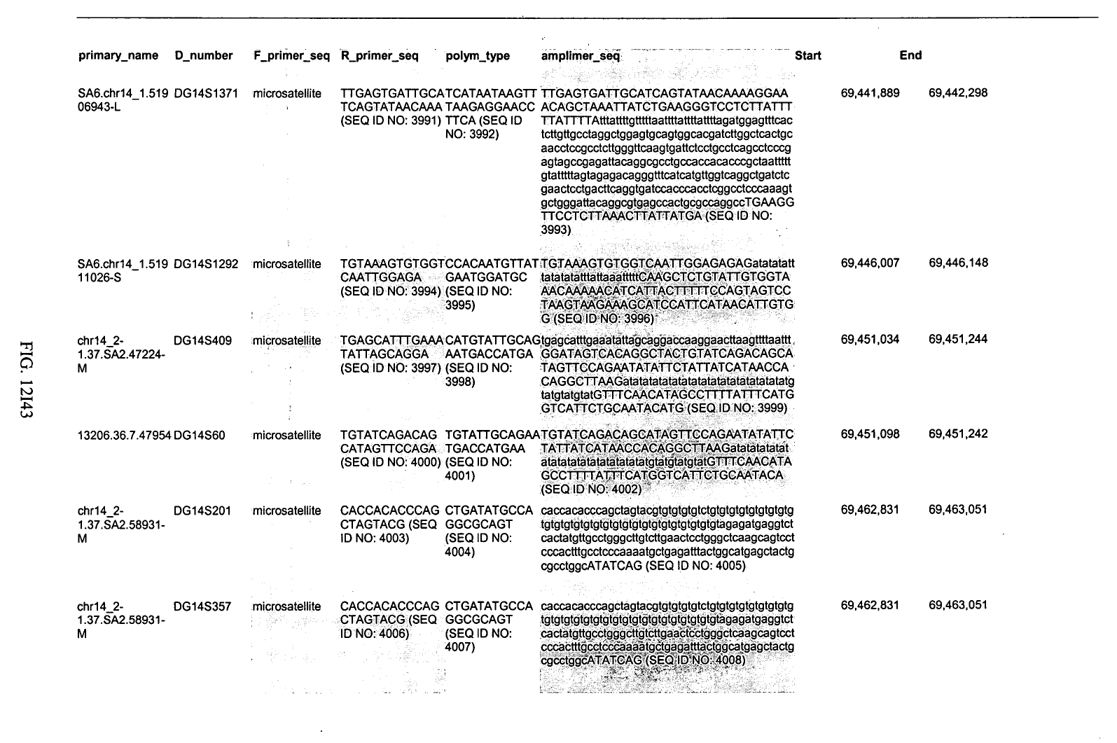

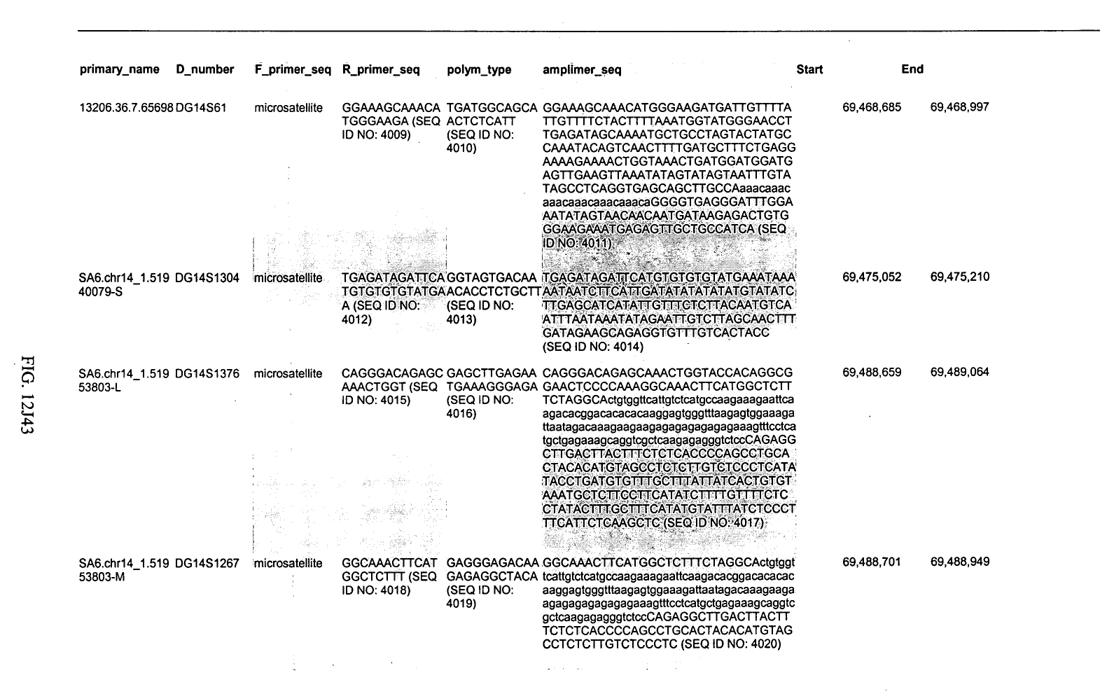

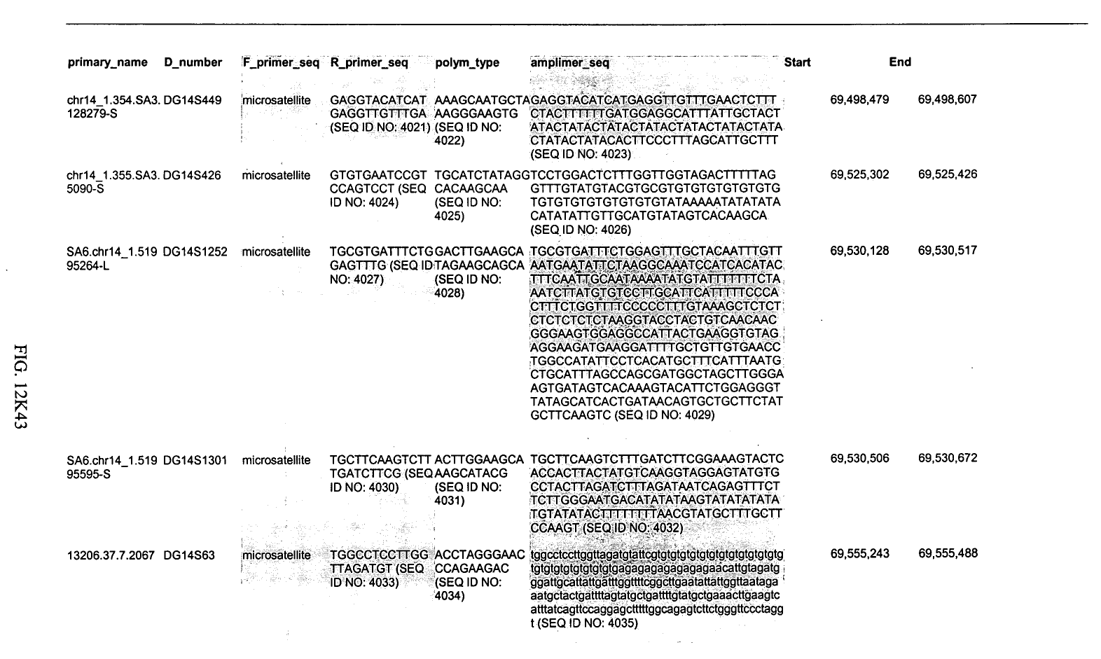

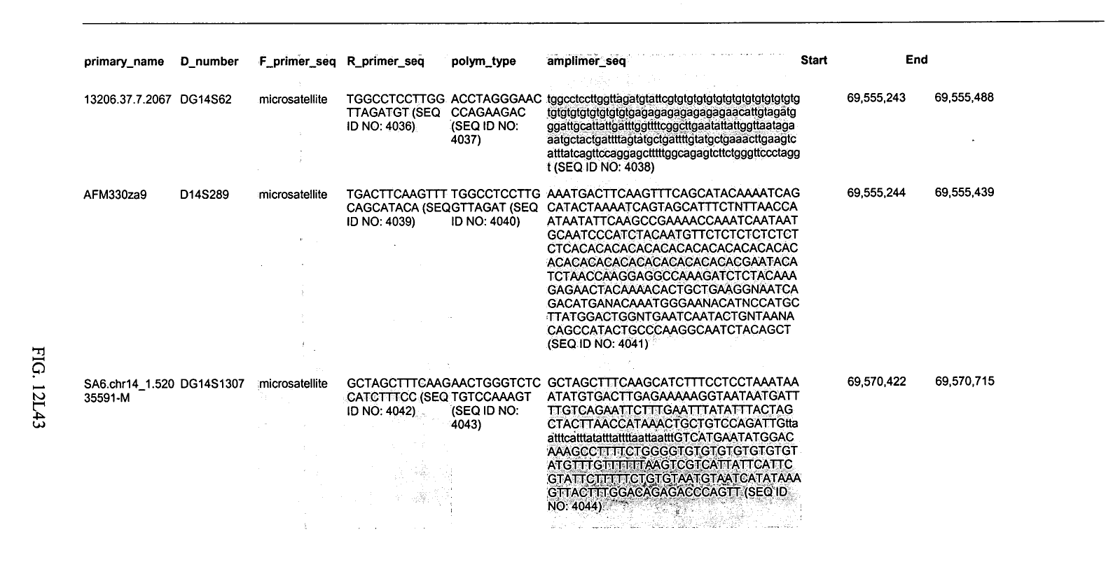

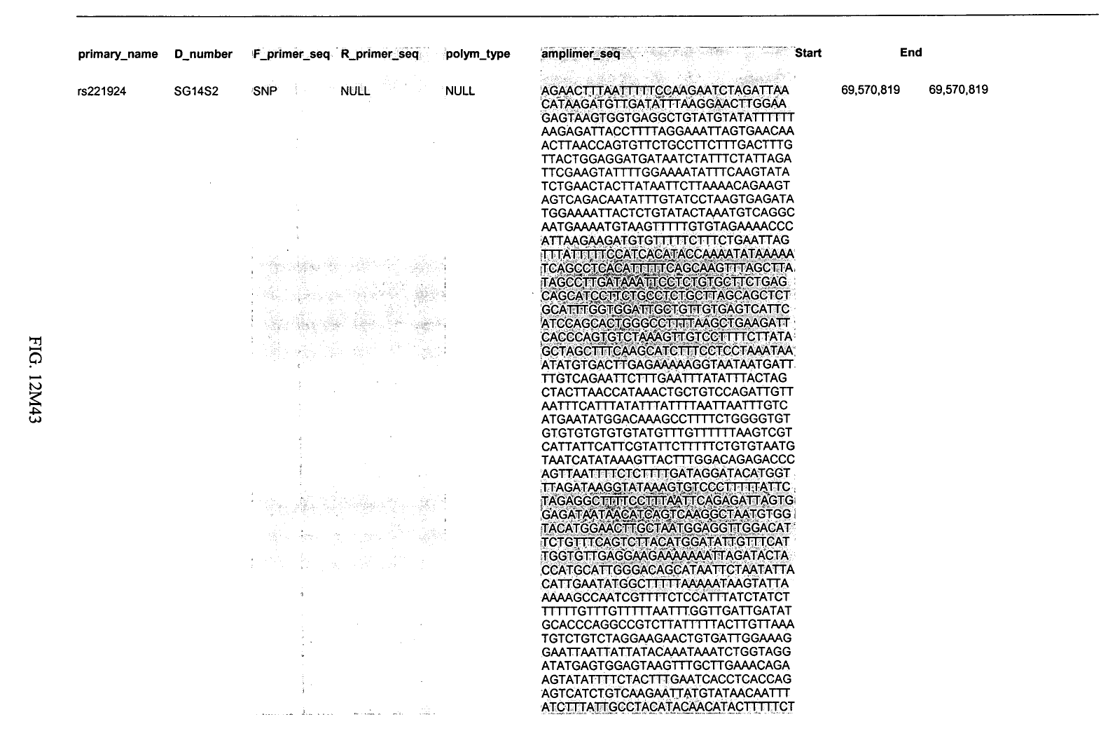

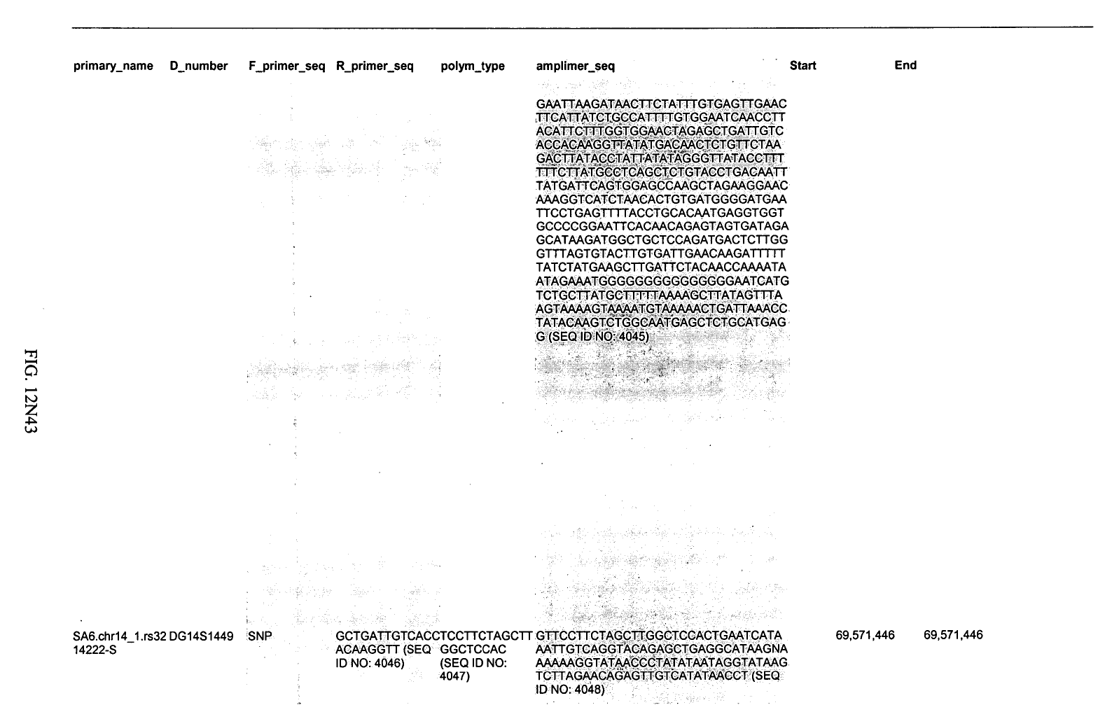

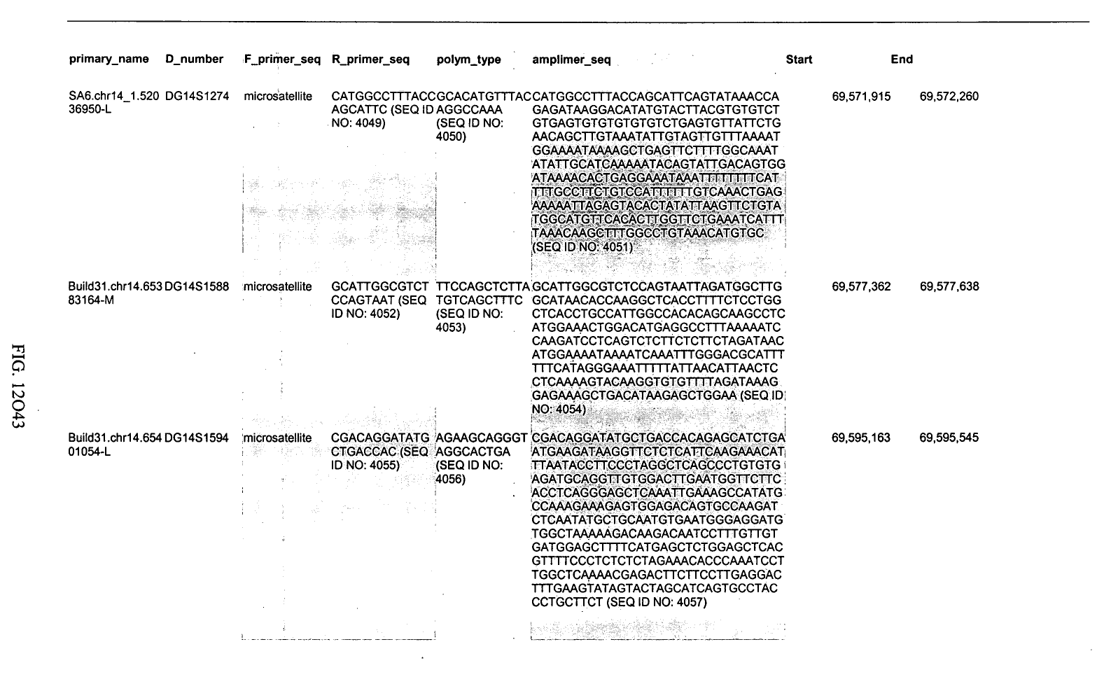

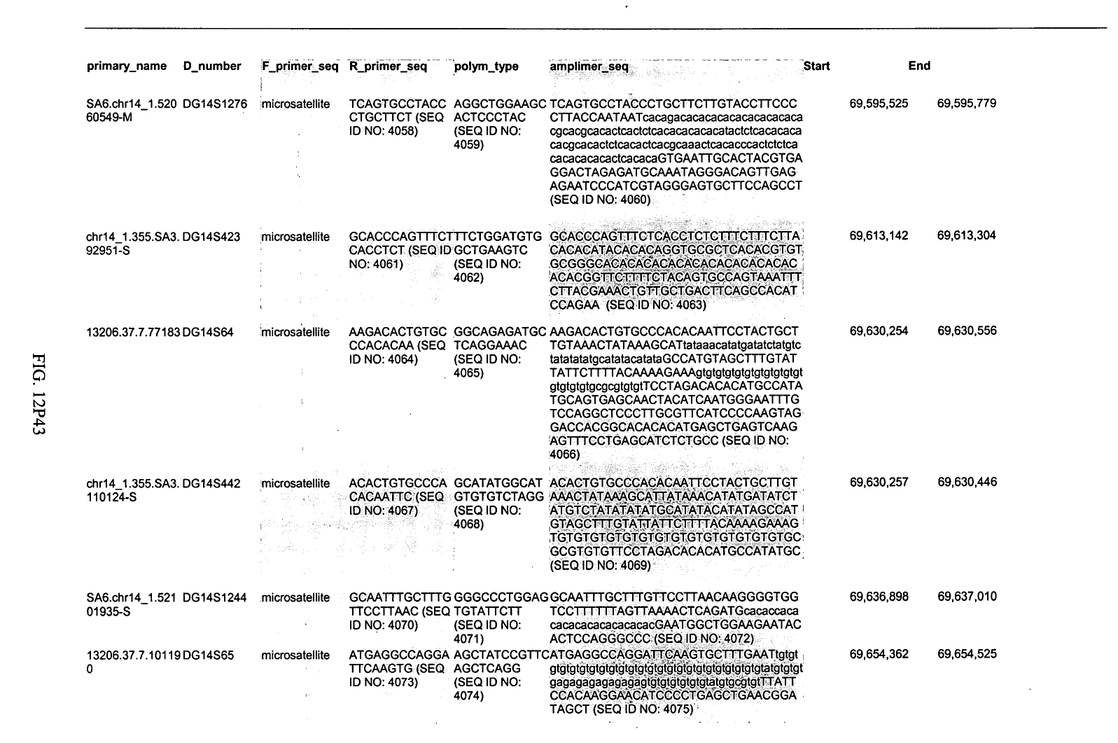

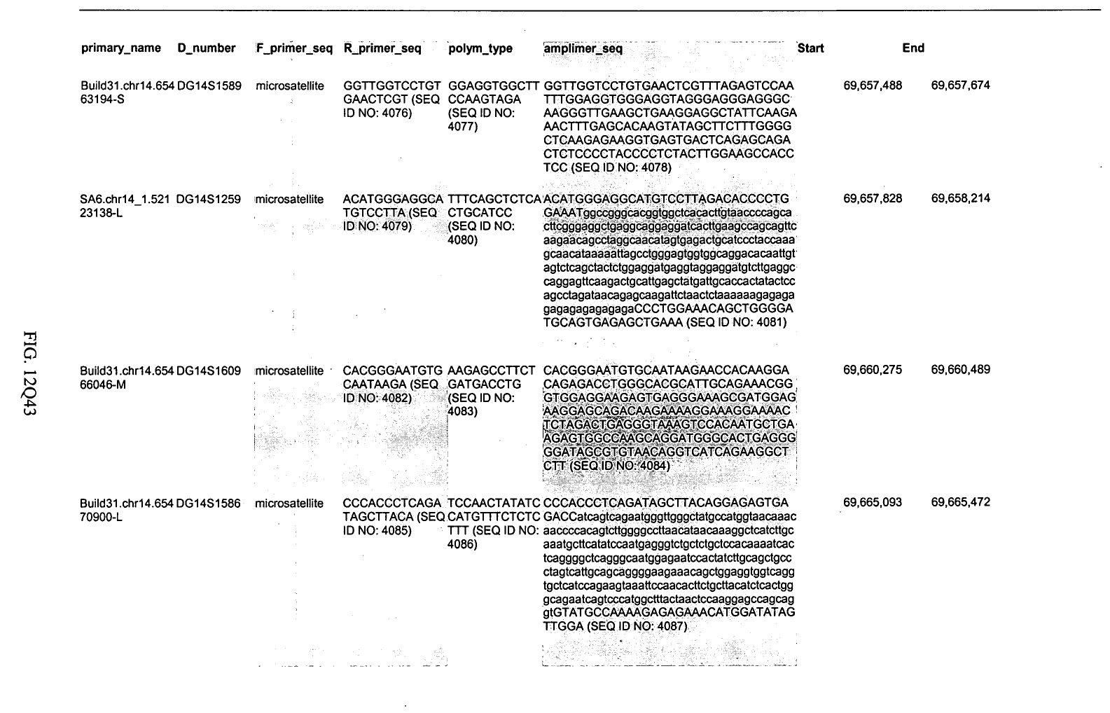

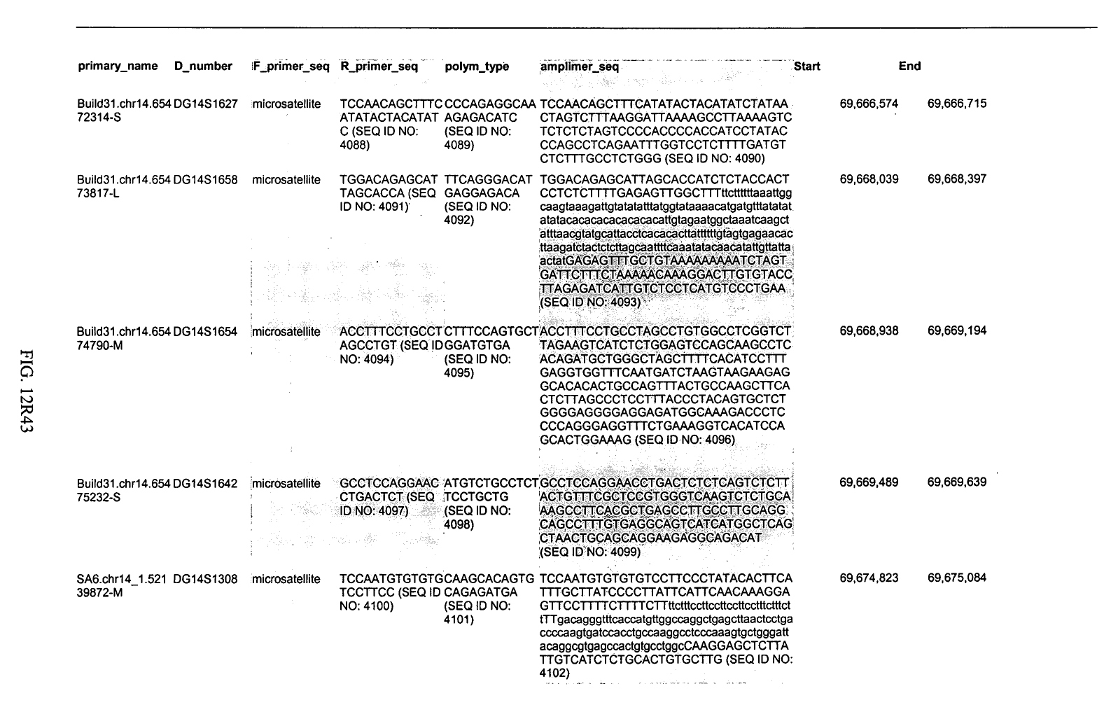

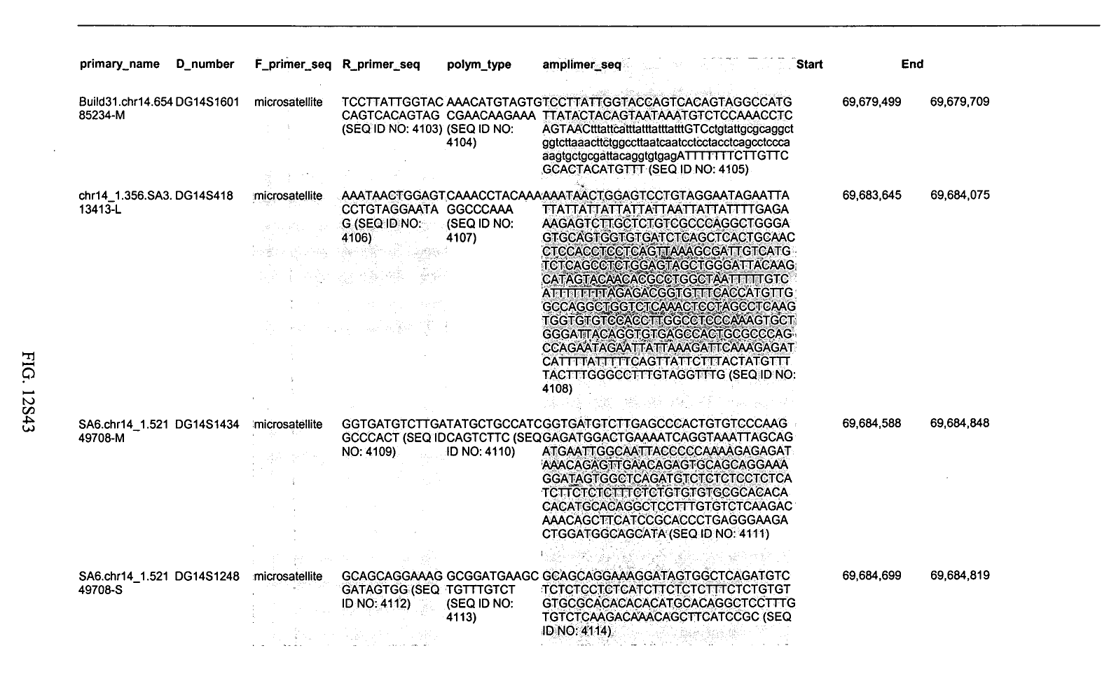

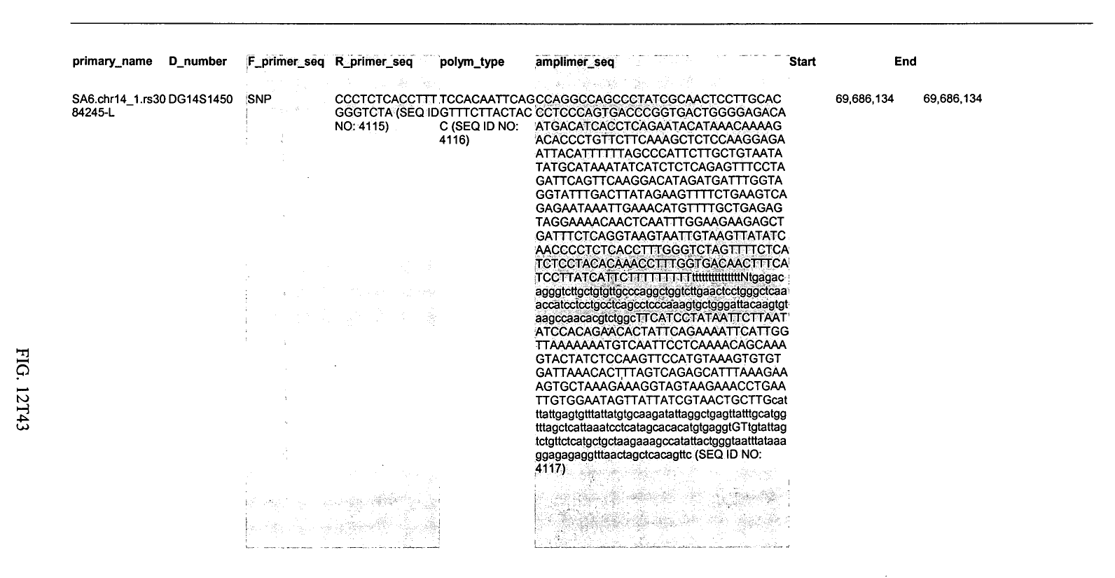

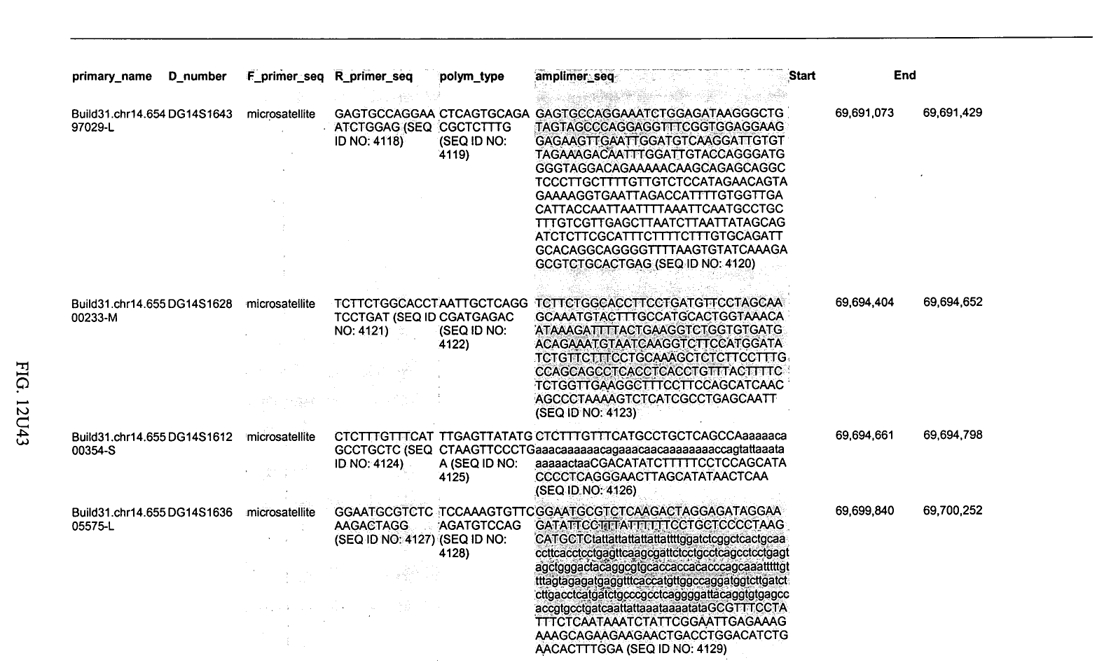

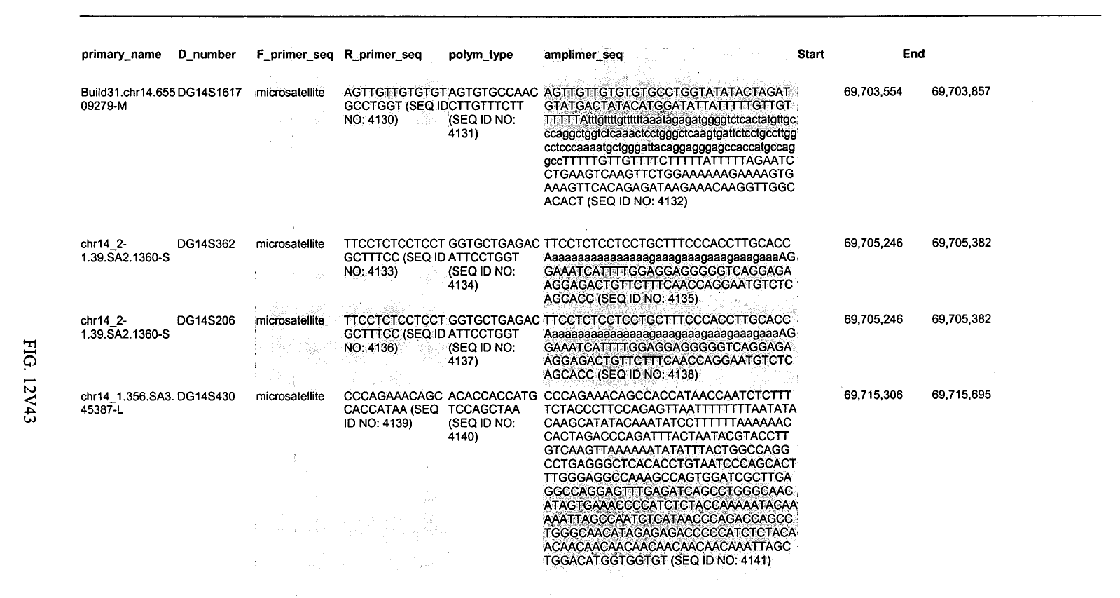

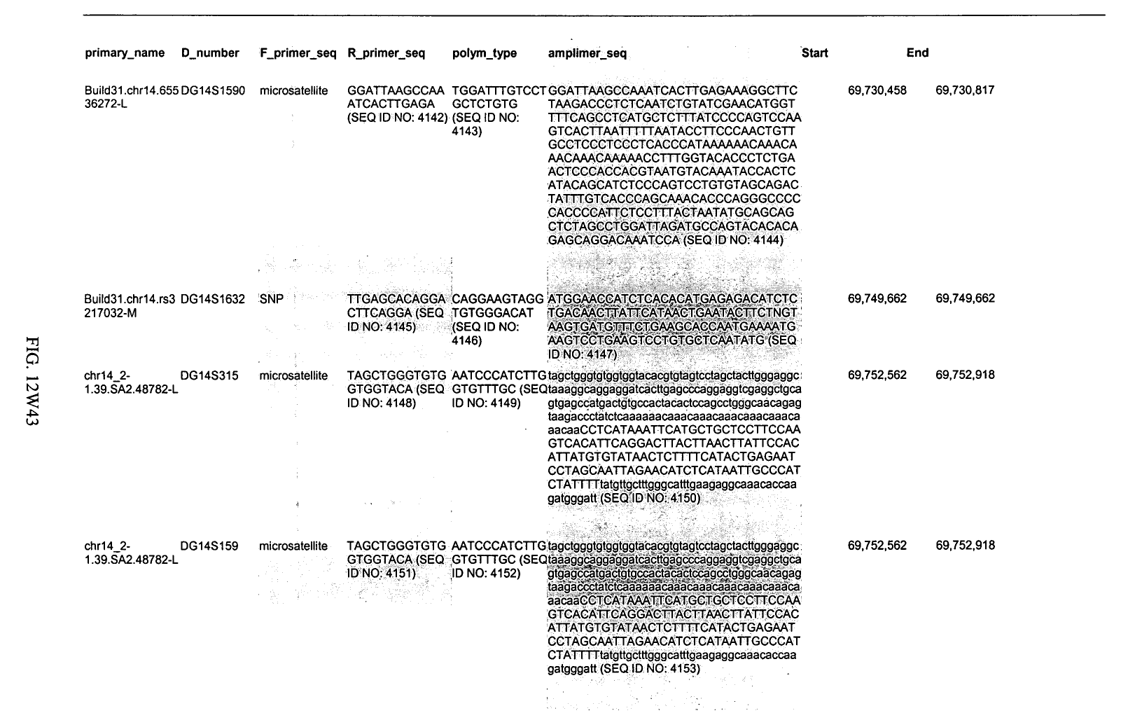

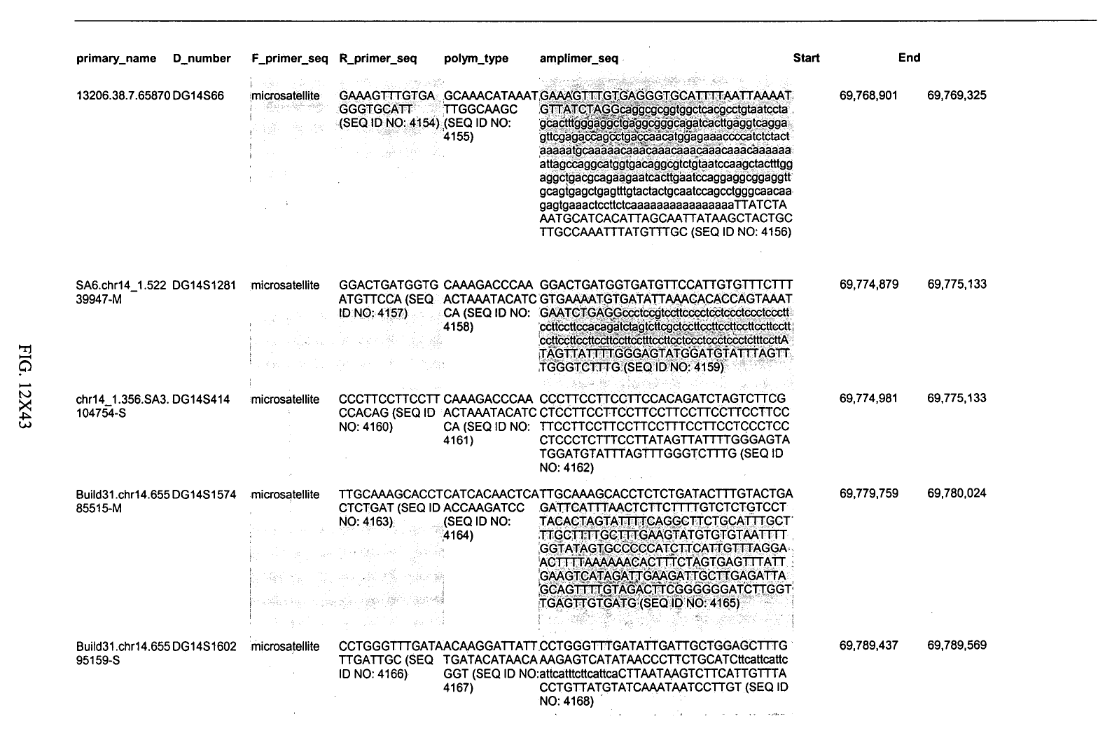

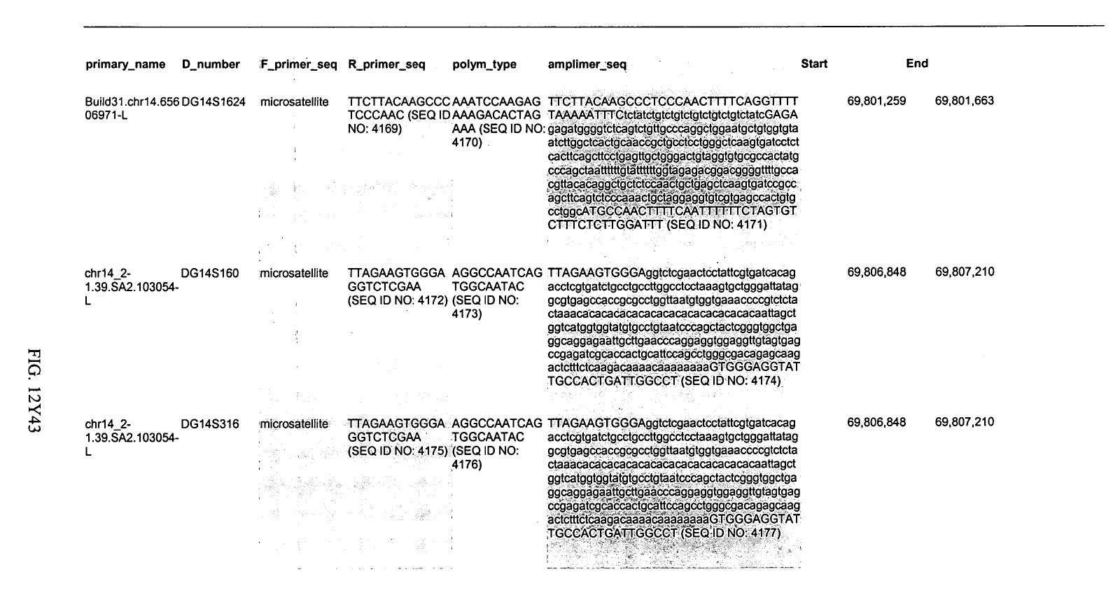

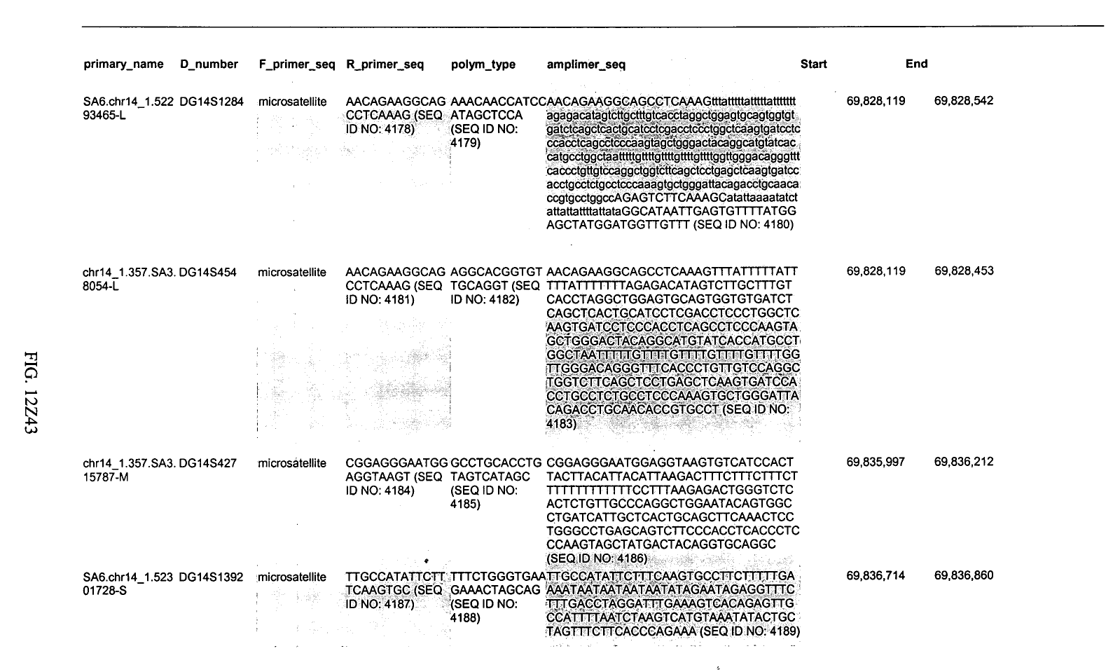

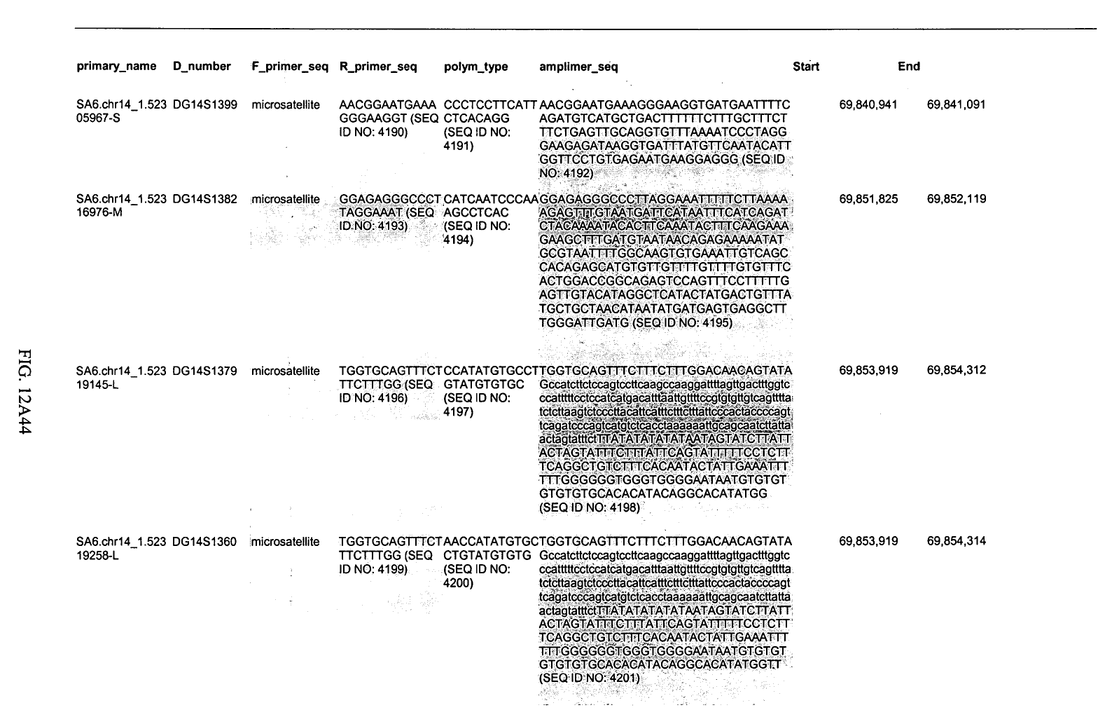

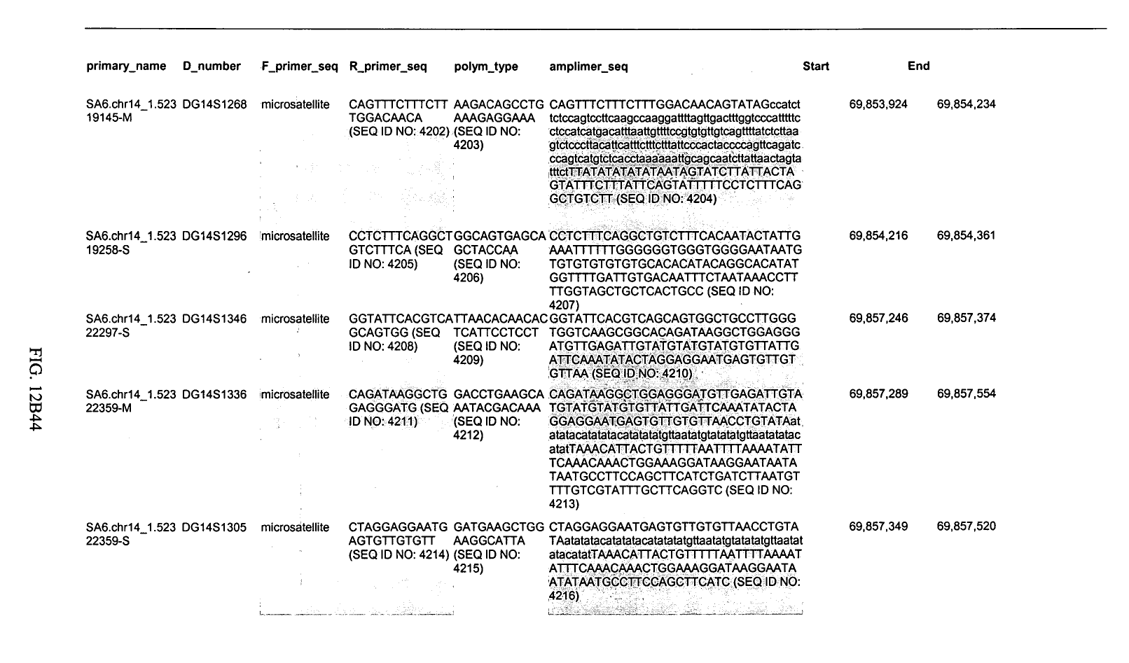

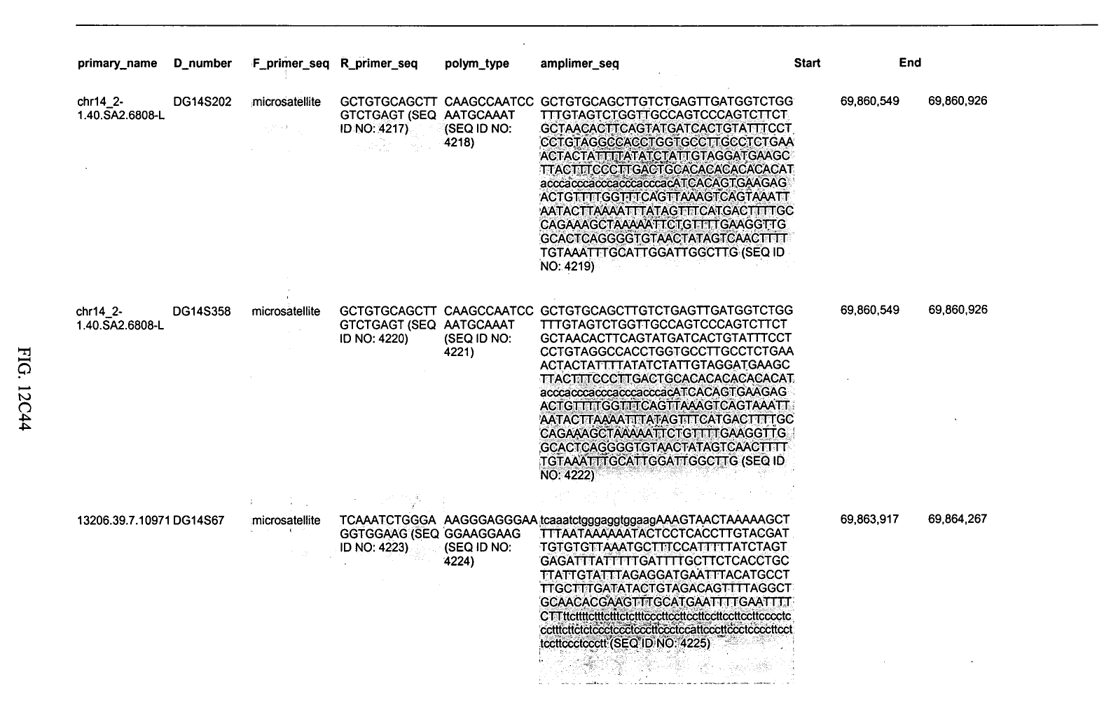

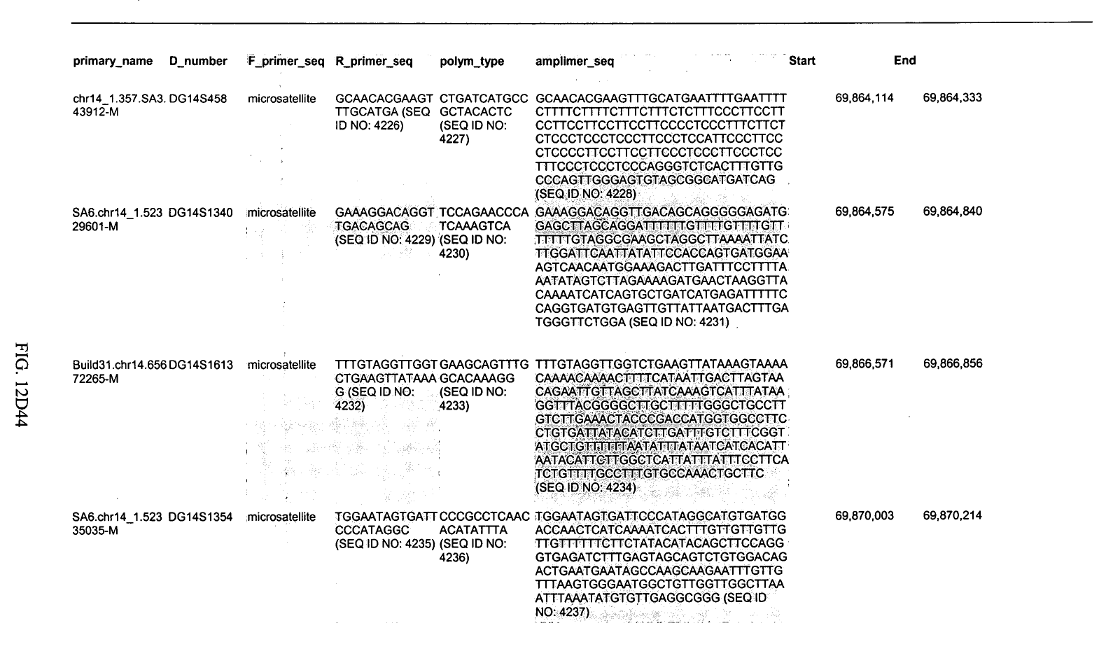

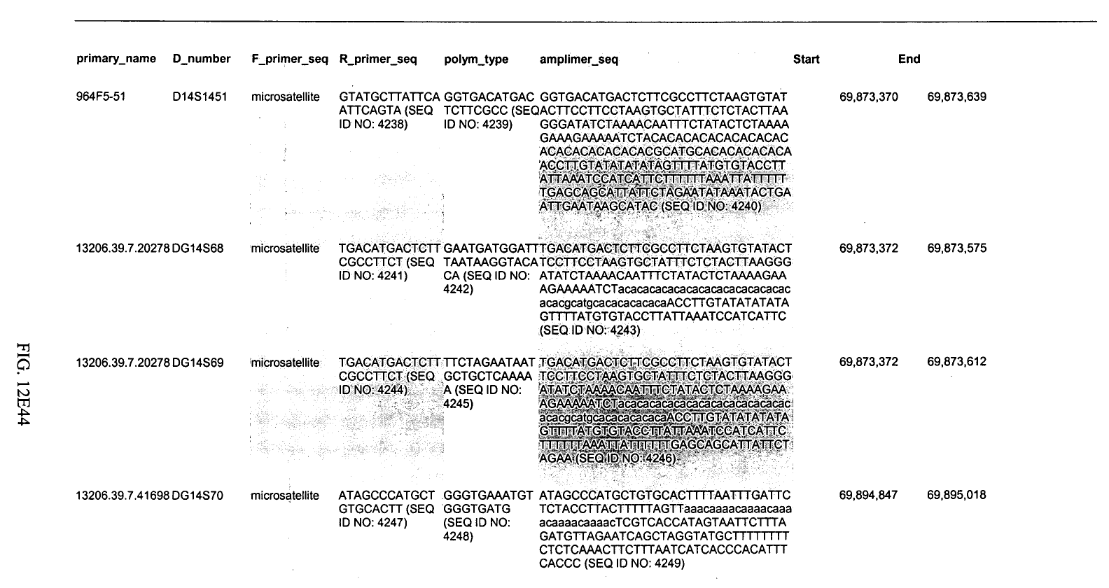

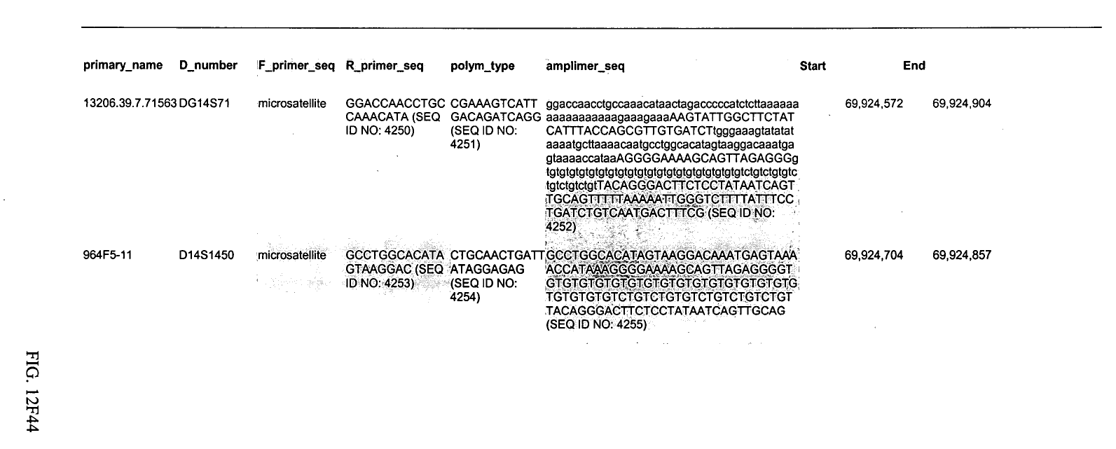

The expression of MAP3K9 in PBM cells from asthma patients vs controls was examined by the above described methods and significantly enhanced expression was seen for the b-isoform (variant b) of the gene in patients compared to control (numbers are listed in FIG. 10).

In conclusion, evidence from the linkage and case-control association studies demonstrate that the MAP3k9 gene is the culprit asthma gene, as judged by positional cloning. In addition, evidence from both asthmatic airway tissue and PBM cells from patients with asthma show that expression of the b-isoform (variant b) of the MAP3K9 gene is enhanced relative to control samples. Collectively, this evidence supports the role of the MAP3k9 gene as a therapeutic target for asthma.

TABLE 1
Haplotype Analysis

| | | | Haplotype |
|---|---|---|---|
| haplotype | marker | type | allele |
| hap1 | DG14S205 | microsatellite | −4 |
| hap1 | DG14S428 | microsatellite | 0 |
| hap1 | D14S1002 | microsatellite | 10 |
| hap1 | DG14399 | microsatellite | 13 |
| hap1 | DG14S404 | microsatellite | 0 |
| hap2 | DG14S428 | microsatellite | 0 |
| hap2 | D14S1002 | microsatellite | 10 |
| hap2 | DG14S399 | microsatellite | 13 |
| hap2 | DG14S404 | microsatellite | 0 |
| hap3 | D14S251 | microsatellite | 2 |
| hap3 | DG14S1300 | microsatellite | 0 |
| hap3 | DG14S420 | microsatellite | 4 |
| hap3 | DG14S1266 | microsatellite | 2 |
| hap4 | DG14S417 | microsatellite | −2 |
| hap4 | DG14S251 | microsatellite | 6 |
| hap4 | DG14S298 | microsatellite | 14 |
| hap4 | DG14S1879 | microsatellite | 4 |
| hap5 | D14S1002 | microsatellite | 10 |
| hap5 | DG14S399 | microsatellite | 13 |
| hap5 | DG14S404 | microsatellite | 0 |
| hap6 | DG14S399 | microsatellite | 13 |
| hap6 | DG14S404 | microsatellite | 0 |
| hap6 | DG14S406 | microsatellite | 4 |
| hap7 | DG14S399 | microsatellite | 13 |
| hap7 | DG14S404 | microsatellite | 4 |
| Hap8 | DG14S1266 | microsatellite | 0 |
| Hap8 | DG14S205 | microsatellite | 4 |
| Hap9 | DG14S420 | microsatellite | 3 |
| Hap9 | DG14S399 | microsatellite | 11 |
| Hap10 | SG14S89 | SNP | 3 |
| Hap10 | SG14S152 | SNP | 3 |
| Hap10 | SG14S174 | SNP | 3 |
| Hap10 | SG14S184 | SNP | 3 |

Alleles #'s: For microsatellite alleles: the CEPH samples (CEPH genomics repository #1347-02) are used as references, the lower allele of each microsatellite in this sample is set at 0 and all other alleles in other samples are numbered in relation to this reference. Thus allele 1 is 1 bp longer than the lower allele in the CEPH sample, allele 2 is 2 bp longer than the lower allele in the CEPH sample, allele 3 is 3 bp longer than the lower allele in the CEPH sample, allele 4 is 4 bp longer than the lower allele in the CEPH sample, allele −1 is 1 bp shorter than the lower allele in the CEPH sample, allele −2 is 2 bp shorter than the lower allele in the CEPH sample, and so on.

### Table 2

TABLE 2
Allele frequencies and significance of haplotypes that capture the relative risk of the
asthma gene, MAP3K9.

| | | | # | | | con. | | | |
|---|---|---|---|---|---|---|---|---|---|
| Hap # | p-val | r | aff | aff.freq | #con | freq | H0.freq | X2 | info |
| Hap 1 | 0.001 | 5.913 | 169 | 0.067 | 134 | 0.012 | 0.042 | 10.349 | 0.813 |
| Hap 2 | 0.0003 | 10.400 | 179 | 0.062 | 135 | 0.006 | 0.039 | 12.954 | 0.810 |
| Hap 3 | 0.0018 | 11598.500 | 111 | 0.032 | 120 | 0.000 | 0.013 | 9.744 | 0.939 |
| Hap 4 | 0.000009 | 2.809 | 212 | 0.128197 | 974 | 0.04974 | 0.069271 | 19.708 | 0.654318 |
| Hap 5 | 0.0001 | 150.592 | 185 | 0.060 | 139 | 0.000 | 0.040 | 14.979 | 0.606 |
| Hap 6 | 0.0012 | 2.784 | 184 | 0.135 | 138 | 0.053 | 0.103 | 10.478 | 0.832 |
| Hap 7 | 0.0006 | 2.831 | 196 | 0.145826 | 141 | 0.0568588 | 0.110 | 11.935 | 0.829 |
| Hap 8 | 0.00049 | 2.435 | 189 | 0.077 | 879 | 0.033 | 0.041 | 12.157 | 0.937 |
| Hap 9 | 0.00067 | 3.653 | 191 | 0.0388 | 731 | 0.011 | 0.016 | 11.562 | 0.989 |
| Hap | 0.0000016 | 2.536 | 166 | 0.158 | 625 | 0.068 | 0.087 | 23.065 | 0.983 |
| 10 | | | | | | | | | |

Hap#—Haplotype number
p-val—p-value
r—relative risk
#aff—number of affecteds (patients)
aff.freq—frequency (of haplotype) in affecteds
#con—number of controls
con.freq—frequency (of haplotype) in controls
H0.freq—frequency (of the haplotype) under the null hypothesis (in affecteds and controls)
X2—chi squared
Info—information content

TABLE 3

| | | | |
|---|---|---|---|
| | Seq. Pos. | | SEQ ID |
| Markers | in NCBI Build 34 | Primer Pair | NO: |

| | | | |
|---|---|---|---|
| DG14S205 | chr14: 69.250346- | F: CATGGGTAAGAGAAAGGGAACA | 3 |
| | 69.250499 | R: AGGTCCCAGCATAGTTCCAG | 4 |
| DG14428 | chr14: 69.267407- | F: GGCAACGTTGACTTCCAGTA | 5 |
| | 69.267644 | R: CAGGCCAGAGTTCAAGACG | 6 |
| D14S1002 | chr: 69.291050- | F: AGATTTTGGATGTATCAGGC | 7 |
| | 69.291217 | R: CAGAAGCAATAGGATGGATG | 8 |
| DG14S399 | chr: 69.316672- | F: GTGTCAGCAACTGCACGATT | 9 |
| | 69.317039 | R: GGCATGGTGGTACATGTCTG | 10 |
| DG14S404 | Chr14: 69.3400521- | F: AAAGCTCAGCCAGAGTCTCAA | 11 |
| | 69.340691 | R: GGCCTTACAGTGCCTAGCAA | 12 |
| D14S251 | CHR14: 69.115638- | F: AAAGGATGAACTATTGGTGC | 13 |
| | 69.115937 | R: TTTACTTGTACCCAGTATGTNTCTG | 14 |
| DG14S1300 | Chr14: 69.134572- | F: TGAAAGGGAGCCTACGTCTG | 15 |
| | 69.134971 | R: TGAATGCGGGAGTAAATAAATG | 16 |
| DG14S420 | Chr: 69.167271- | F: AGGGTGAGAGACTGCTCTGG | 17 |
| | 69.167488 | R: GGAGGGAGGGAAGAAAGAGA | 18 |
| DG14S1266 | Chr14: 69.182502- | F: GCCAAAGAGAGAGGCAGGTA | 19 |
| | 69.182636 | R: CCAGATTGCTTCCCTTGGT | 20 |
| DG14S448 | Chr14: 69.216836- | F: CAAAGGATTAGCTTTACGGTATGT | 21 |
| | 69217046 | R: TCATGGCTGTGGGACAGTAG | 22 |
| DG14S417 | chr14: 69.083564- | F: GCCCTAAGGACTACACACAGA | 23 |
| | 69.083917 | R: GCCTTCCAACCATGAACAAG | 24 |
| DG14S1879 | chr14: 69.254098- | F: ATGGGCAAAGAAGTGGCATA | 25 |
| | 69.254375 | R: TACAGGAGACTGCCACCACA | 26 |

TABLE 4

| | | |
|---|---|---|
| | SEQ | |
| CEPH | ID | |
| (bp) | NO: | Amplimer |

| | | |
|---|---|---|
| 165/ | 27 | CATGGGTAAGAGAAAGGGAACATTTACATTTTGGGATCACGT |
| 165 | | AAGTAAACCAGTTCACATGATATttcattcattcattcattc |
| | | attcattcattcCTTTTATCTATacatttattgaggacccta |
| | | ctatgcacctggaactatgctgggagct |
| 244/ | 28 | GGCAACGTTGACTTCCAGTAAGAAAGGTTCTCTGATCTTTTT |
| 244 | | TTCTCTTTTCTTTTCTTTTCTTTTCTTTTCCTTTCTTTTCTT |
| | | TTCTTTTCTTCCAGGGTCTCAGTTTGTTGTCCAGGCCAGAGT |
| | | GCAGTGGCGCAATCTCGGCTCACTGCAGCCTCAACATCCTAG |
| | | GCTCAAGCCATCCTCCCACCTCACCCTCCCGAGTAGCTGGAA |
| | | GTACAGGCTCGTCTTGAACTCTGGGCTG |
| 157/ | 29 | AGCTCTNCCTGGTTCACTAGCAGACTGTCTTTGGACTTGAAC |
| 157 | | TTCTCTTTTTCCTACATCTCTGGCCAGTNAGCCTNCCGTGTC |
| | | AGATTTTGGATGTATCAGGCTTCCACAATTGGGTGAGGCGAT |
| | | TCCTTTAGAATAAATCTCTTTTACACACACACACACACACAC |
| | | ACACGTGTACCCACACACACGTGCATATGCACACACACACAC |
| | | GNACACATCCATCCTATTGCTTCTGTTTCTNTGGAGANCCCT |
| | | NATACAAATTNGCATGTCATTAAATATCACTAGTGTTGTAGC |
| | | T |
| 375/ | 30 | gtgtcagcaactgcacgatttctccctattcagccagtaggg |
| 379 | | aatctagagcaattctattatctagtacaactttagcaagag |
| | | aatttaaagtctgttatgcaaccatagcctttgcaatagaat |
| | | ctgctatagagatgattataaggaatttccttccttccttcc |
| | | ttccttccttccttccttccttccttctttctttctttcttt |
| | | ctttctttctttctttctttctttctttctttcttctttctt |
| | | tttctttctttctttccttctttGAGACAGGGTCTfGctcaa |
| | | cctcccaggctcaagcaattctccctacctcagtctcccaag |
| | | tggctgggagtacagacatgtaccaccatgcc |
| 174/ | 31 | AAAGCTCAGCCAGAGTCTCAATFCCTATAACCTCCyrCccat |
| 178 | | ccatccattcatccatccatctatccatccatccatccatcc |
| | | atccatccatccatccaGCTGATGGAttaataaatttatatc |
| | | aagcacttactctgagcaaggcattttgctaggcactgtaag |
| | | gcc |
| 298/ | 32 | CTACAGAGCAATTAAAAAAGGATGAACTATTGGTGCATTTAA |
| 300 | | CAACTTGGATGCATTGCAAGGAAATTATGCTGAGTGTAAAAA |
| | | GCGCCTTCCAAAGGATTTCATGCTATGTGATTCCATTTATAT |
| | | AATATTTTCAAAATGACAAAATTTTAAAAATAGAGAACAGGT |
| | | TAGCAGTTGTCAGGGGTTAAAGAGGAAGTGGTGGGACAGGAA |
| | | GGAGATGATAGAGGAACCTACACACGATAAAACTGTATATAA |
| | | CTAAACACACACACGCACACACACACACAGACACACANACAG |
| | | ANACATACTGGGTACAAGTAAAGCT |
| 407/ | 33 | TGAAAGGGAGCCTACGTCTGACCCCTAGACTTGCCTTGTTTT |
| 407 | | CCAAGTCtcacagaatattagggctggacagaagcttgaagc |
| | | tcagtagttcaaccctcttttctttcagatgagagatcaagt |
| | | ccagagatgattaagagatttattcaaggtcacacagatagt |
| | | aagcagcaaatctgaactggaactcaggtcagtttcctgaca |
| | | acaaacaatactctttccactgaaccaAAGTATAGTTCCAAA |
| | | atatatatacacacatatatgtatatgtgtgtacgtgtgtgt |
| | | atatatatatatatgtgtgtgtgtgtgtatatatatatatGC |
| | | TGTAAAATTGTTCTCTTCTAAAGAAAGAAGGAGCATAGTACT |
| | | CATTTATTTACTCCCGCATTCA |
| 243/ | 34 | AGGGTGAGAGACTGCTCTGGAGCTCAGTGAGGGACCTCAGGG |
| 243 | | TTACACAAGAAGGGACTCTGCGCTGGGTTCCAGGCCTGATTC |
| | | TTTTATTTTCTCTTTTTCTTTCTTTCTCTTTCTTTTTCTTTC |
| | | TTTCTTTTCTTTCTTTCTTTCTTTCTTTCTTTTCTTTCTTTC |
| | | TTTCTTTCTTTCTTTCTTTCTTTCTTTCTCTCTCTTTCTTCC |
| | | CTCCCTCC |
| 144/ | 35 | GCGAAAGAGAGAGGCAGGTACAttagggtgaaccatatgaaa |
| 144 | | tagctgatattcgactgtttttgacgtaaaaCCGTACGTATA |
| | | TTTcacacacacacacacacacacTTTTCTGCACCAAGGGAA |
| | | GGAATCTGG |
| 210/ | 36 | CAAAGGATTAGCTTTACGGTATGTGAATAATAGCTCAATAAA |
| 210 | | GCTATTATTTAAAATAAACGTGCACACACACAGACACACACA |
| | | CACACACACACATACAGAGCTCACTTTACAACACATCAGGAC |
| | | AATAACTCGATGGACTGATATATAATGATAAACTACAGTCCT |
| | | TTCTTTTGTATACTAAGGTGATGCTACTGTCCGACAGCCATG |
| | | A |
| 358/ | 37 | GCCCTAAGGACTACACACACACACACACACAGAGAGAGAGAG |
| 362 | | AGAGAGAGACAGAGAGAGAGAGAGAGAGAGAGAAAATCTATT |
| | | AGAACTAATCAATAAATTTAGCAACATTGCAGGATACAAAAT |
| | | TAACACACAAAAGTCAGTCGCATTTCTATACACTAACAACAA |
| | | ACAAATTCGAAAAGGAAATTAAGAAAACAATTCCATTTATAA |
| | | TAACATCAAAAACAAAAAAAATTAGGAATAAAATGAAGTAAG |
| | | AAGGTGAAAGACTTGTACAGTAAAAACTATAAAACATTGCTG |
| | | AAAAGAGTAAAGAAGATAGAAATAAATAGAAAGGCATTTCTT |
| | | GTTCATGGTTGGAAGGC |
| 286/ | 38 | ATGGGCAAAGAAGTGGCATAACACTTCTAAGATAATTTTAAG |
| 286 | | CCTGGGCCACAAAGCAAGAGTCCCATCTCTATTAAaataata |
| | | ataataataataataaAATTTTTTAAAAAAAggcttggtgca |
| | | ctggctcacgcctgtagtcccagaactttgagagactgaggc |
| | | aggtggatcacctgaggtcaggagttcgagaccaggctggcc |
| | | aacatggcgaaaccccacctctactaaaaatacaaaaattag |
| | | ccaggtgtggtggcagtctcctgta |

TABLE 5
MAP3K9 SNPs in Chromosome 14q24.2

| Position¹ | Marker name | Public name | Seq Id no. |
|---|---|---|---|
| 69.054355 | SG14S14 | rs1007402 | 39 |
| 69.65892 | SG14S130 | rs2332431 | 40 |
| 69.074372 | SG14S131 | rs2185146 | 41 |
| 69.079703 | SG14S132 | new² | 42 |
| 69.097983 | SG14S178 | rs1572678 | 43 |
| 69.102942 | SG14S141 | rs4902835 | 44 |
| 69.107276 | SG14S181 | rs1953796 | 45 |
| 69.117148 | SG14S134 | new | 46 |
| 69.117741 | SG14S182 | rs1959482 | 47 |
| 69.118774 | SG14S168 | rs9805907 | 48 |
| 69.119834 | SG14S77 | rs1205235 | 49 |
| 69.122800 | SG14S76 | rs1205233 | 50 |
| 69.128829 | SG14S96 | rs3742849 | 51 |
| 69.129320 | SG14S61 | rs8688 | 52 |
| 69.140462 | SG14S116 | rs4899366 | 53 |
| 69.157716 | SG14S118 | rs4141096 | 54 |
| 69.169752 | SG14S119 | rs4902841 | 55 |
| 69.169812 | SG14S268 | rs7144832 | 56 |
| 69.179795 | SG14S409 | rs8006539 | 57 |
| 69.181464 | SG14S242 | rs3742848 | 58 |
| 69.186468 | SG14S94 | rs2286053 | 59 |
| 69.186468 | SG14S411 | rs2286053 | 60 |
| 69.188592 | SG14S412 | rs11844774 | 61 |
| 69.189493 | SG14S97 | rs3829955 | 62 |
| 69.189493 | SG14S308 | rs3829955 | 63 |
| 69.194410 | SG14S413 | rs8011047 | 64 |
| 69.199037 | SG14S147 | rs4899368 | 65 |
| 69.199066 | SG14S98 | rs4902843 | 66 |
| 69.199066 | SG14S314 | rs4902843 | 67 |
| 69.205290 | SG14S165 | new | 68 |
| 69.205421 | SG14S148 | rs2332457 | 69 |
| 69.205421 | SG14S315 | rs2332457 | 70 |
| 69.205770 | SG14S99 | rs10129495 | 71 |
| 69.205770 | SG14S317 | rs10129495 | 72 |
| 69.210078 | SG14S166 | rs4902847 | 73 |
| 69.210257 | SG14S149 | rs4902848 | 74 |
| 69.212106 | SG14S120 | rs1476609 | 75 |
| 69.217954 | SG14S108 | rs3814872 | 76 |
| 69.217954 | SG14S318 | rs3814872 | 77 |
| 69.218041 | SG14S100 | rs11621281 | 78 |
| 69.236948 | SG14S247 | rs2107666 | 79 |
| 69.237009 | SG14S173 | rs2158531 | 80 |
| 69.237036 | SG14S248 | rs2158530 | 81 |
| 69.243542 | SG14S415 | rs8019513 | 82 |
| 69.244739 | SG14S249 | rs1034769 | 83 |
| 69.244739 | SG14S416 | rs1034769 | 84 |
| 69.249713 | SG14S151 | rs2107665 | 85 |
| 69.249922 | SG14S90 | rs2158529 | 86 |
| 69.253468 | SG14S417 | rs2051857 | 87 |
| 69.257386 | SG14S101 | rs4902857 | 88 |
| 69.261096 | SG14S89 | rs2023955 | 89 |
| 69.262833 | SG14S152 | rs4902858 | 90 |
| 69.276792 | SG14S175 | rs4902860 | 91 |
| 69.284136 | SG14S125 | rs4899373 | 92 |
| 69.286711 | SG14S174 | rs2332467 | 93 |
| 69.287341 | SG14S177 | new | 94 |
| 69.296950 | SG14S74 | rs1018976 | 95 |
| 69.299388 | SG14S186 | rs992941 | 96 |
| 69.299524 | SG14S72 | rs979536 | 97 |
| 69.299704 | SG14S159 | rs1024512 | 98 |
| 69.300316 | SG14S75 | rs1024510 | 99 |
| 69.307253 | SG14S184 | rs4899377 | 100 |
| 69.338169 | SG14S143 | rs2810084 | 101 |
| 69.344621 | SG14S156 | rs36561 | 102 |
| 69.349626 | SG14S71 | rs714421 | 103 |
| 69.354602 | SG14S1 | rs36555 | 104 |
| 69.360375 | SG14S161 | rs2158537 | 105 |
| 69.361945 | SG14S129 | rs917067 | 106 |
| 69.363621 | SG14S45 | rs2240341 | 107 |
| 69.371260 | SG14S163 | rs2526874 | 108 |
| 69.378439 | SG14S115 | rs12892688 | 109 |

¹Position numbering relative to Build34 (Database name)
²Detected by sequencing

| | |
|---|---|
| Se- | |
| quence | |
| ID no. | Amplimer [sequence variation] |

| | |
|---|---|
| 39 | TTAAAAGTGAATACTAATAGTTACCCTGCATGTCAAATTTGC |
| | ATAAACCCAAACAAAACTCCAGTTTTTCTTCTAAAAGTTCTA |
| | CTCTGCACTTCATGATCCACAGTTTGAAAGAACTGTTCATTG |
| | CAAAACTTGTATTTGGAAAAAGTGC[C/T]ACCTTCTTAAAAT |
| | AACTTACTTCAAGTGTTCTAATGTTAGCCTCTGCATTTTGAC |
| | CACTTCATTATTACATGTTCTGTCATAAAAAGGATTACTTCT |
| | TTCTGAAAAGTAATCCAGGACACTACCACTGTTCAAAATAGG |
| | GATCCAAGAGCTGTCAACCCAAGAAATTCCCAGCAGATTGTC |
| | TATGGGAAAGAAAACAAAATGATAAAGGATTAGAAAAATAGA |
| | AAGAATCCAATCCAACA |
| 40 | AATAATGGGAAAGGCTTCTATTCTTAACATGCTTAGTTTGTA |
| | TTTTAGATAAATAAGACATACAGGTATACGTATATACACTCA |
| | AAGATAAAACAAGTAATTCATAATTTTTGTTGCAAATCTATT |
| | ACTCCCCTTACCAGAAAACTATATTTACACATAAGAATTTGT |
| | CAGATCTCAAAA[A/G]CAAGATTGGAAATCATAGTGATCATT |
| | GTATATTTTAATATAGAATGAAAATAAATTTGACTTTGAGGT |
| | ATAGTTGAAATGGAAATAAATCAGATGTGAAATTAGATTGAA |
| | TAAGTTCAAAGTGGTAATGAAAATAAGCCTTGTATGTACAAA |
| | TTCAATAAAAGTATTTGTCTCCCTACAGGCATAGCAAACATA |
| | GGTGGAAAGATTTTACAAAGGTATTATGGGG |
| 41 | GGATGTTTAAGCAGAAACAATGAAAGACTAGTAGAAGTTAGC |
| | CAATGAAAAGGAGAGGTAAGAAAATGCAAGCAGAGGGAAGAG |
| | CATGAACCAAAGCCAAGAGGCAAGAGAGAAGAAGAAAATGGA |
| | AAATGTTCGGAATAGCTGGAACATCGAATATATGAGAAGACT |
| | GGATGAGAGTAGATTGTC[A/G]CAGAAATCAGAAGTAAGATC |
| | ATTGTAAACTATGCCAAGGAATTGAGAGCAGTGGGACACTAA |
| | AGAAAGATTTTAAGGAGGCAAGTGACAAGGAGGCAAGATTTC |
| | AATTTAGAAATGTGTTATCATAATCTAAGCCTGAAAAGATGA |
| | TGACCTATGCTAAGTAAGTAACAGTAACAGTAAAAAGAAATG |
| | AACTAAAATGATAGATATTT |
| 42 | AGATAGAAATTATAAAAAAAGAACCAAACAAATTCTGTAGCT |
| | GAAAAATATAATAACGGTAGAAAAAATAGTAGAAGGATCCAA |
| | CAGCAGACTTGATCAGGCAAAAGAAATAGTCAGCAAACTCAG |
| | AGACAGATCATGTAAAGTCACTGAGTCAGAGGAGCAAGAAGA |
| | AAAAAAATGAAGAAAAGTGAAGACAGCCCAAGGGATTTACAA |
| | AACACTATCAAGTAGCCTAATATATG[C/T]ATTATTAAAATC |
| | TCAGAAAAAGAATGAGAGAAAGGGACAGAGAGCTTATATGAA |
| | CAAATAATGGTGAAAACTTCCTAAATCTGAGGAAAAAGAAGA |
| | ACATACAGACTCAAGAGCTCAATTCAGAACCCTAACCAGAAT |
| | AA |
| 43 | GGTGTATCTTCGCCTGTAGACTGGGAGTTTCTTGAGGTCAGG |
| | GGAGTGACTTGCCTATCTTTGCCTCTTAGCCTTAGCAGATCC |
| | CCAGTAGACAGCTGACACTCAGAACCGACCAAAAGAAGGAAG |
| | GAATGGGACCTCCTGAGGAAGGGCACAGAACTGTCCTGTAAA |
| | TGGAAACTAGACCGGAGGGACGC[C/T]AAGCCGATGACCCAC |
| | AGTAACAGGTGTTCTCCATCTCTGATGTCTGTGATTCATCCT |
| | GCACAGCTGCGCGCACACAGCACACGCATACACAGGCTCCCA |
| | GAATACGGCGCCCCACACGCTGTACCGCCCGTCGCGTTCTGG |
| | ACGCCCCGGCGACCACACACACCGAGA |
| 44 | GATGACTTGAGGGGACGCATGAAGGGTGTGCAGGCAATTTGC |
| | AAAGGGCACCAGGTTGTGGTCTCTTCTGGCTGGGATTTCCTT |
| | TTGGTTGGAAGAGATCTGGCCAGTTGTGCTGGGCCCCTGGGT |
| | GT[A/G]ATTTTTGCGTATCTTGTTTTCCAGGAAATTCAAAGA |
| | AATAGGTAGGGATCGTTCTGTTTCTACGGAATTTTGAATGAT |
| | CCTGATACAATGATTAGAAAAGATATCCTGACCTTCCTGGGG |
| | AGACATAGGAATCTGATCTTTACAGAGGCTACAAAGGAAGCA |
| | TGTATTGGCCTGTTGATGGACTCTTCTTGACATTGAACAAGA |
| | CACAGGACTCCCCTGGGGCCTTGGAAT |
| 45 | CACTGCACGCCAGCGTGGGGGACAGAGCAGGACACCATCTCA |
| | AAAAAAAAAAAGAATATACTTGATGAGGAAAATAAAAGGGGA |
| | GTTCTTTGAAGGCTAAAATTTCCCCAATTCTCTTCAGACATC |
| | TTCTTAAGAA[C/T]GTAGGAGAAGTGGGGCTTAGTGAAAAGG |
| | GACCTGGACTGCAAGTTCTGTCTCTAACTTTCTGTGTGCCTT |
| | GGATCAAAGAATTTTACCTCTTGGACCATGGTGTTCTGATTT |
| | TCAGAAAATTAAGGGTTGAATTCTAAGATCTCTAATCTCCTT |
| | TCCAGGTCTTAAAGAAAAATTCTCCACATCATTCTTTCTGAA |
| | CTGTGTGGCCATTACATGGAATAGAGAAGTTCTATGTGTG |
| 46 | GCACACATGTGCAGATTTCCAGCTCTAGGACTTGGCCATCTG |
| | ATACCTGTCTGCCCTTGTGGGTAGTGGGTGAAAATCTACTTT |
| | AGATAGAAAATCTACTTTAAATTACGCTGTAGCTTTGTTTTG |
| | TACCACAT[A/G]TTGTATATAGGACTCAAGATGGAAATTTTC |
| | TCCAATTCATGTACACATTGCTGGGCAAACCATATTTGATAA |
| | GTGACTTTCTAAAGAACTAGTATTTATGAGAATCCATTTTTG |
| | TCCCAGCACTGTGAGAAGCATGTAATGTGCATTAATTATCTC |
| | ATTTCATCATCATAAGCGTTCCCCCAAAGTTCCTATTTAACA |
| | TGGGAAGAATAAGACCCAGGGTGATCATATAACTCGCCCTCG |
| | G |
| 47 | TTCTTCCACTTTACTACTATGTCATTGCTTCTTGAAATAAAT |
| | TTGAAAGCAAATCAAATAATTTGGCAAACTTTTGGGAAAATT |
| | TTCCCCTAGGAACCCAATGGCCATTTCCTGGGCCCATCTCAC |
| | AAAGCAGTGACTAGCAAGGTATATTAGTTTCCTAAGGCTGCT |
| | GTAAT[A/G]TCACACACTGGGTGACTTAAAATAACAGAAATT |
| | CATTGTCTTGTCATTCTAGAGGCTAGAAGTGAAACATCAAGG |
| | TGTCGGCAGGGTTGGTTCCCTCTGAGGGCTGTGAGGAAGAAT |
| | CTGTTCCAAGCCTCTCTCCTAACTTCTGGTAGCCCCAGGCAC |
| | TCACTGGCTTTGTAGATAGCCGTCTTCCTAGATCTTATTGCA |
| | TCATCTTT |
| 48 | GCTGGCAGTGGAGGAATCTGTACTTGGGAGGGGTGGTTGGAG |
| | ATTTGCAGCTTCAATTCTTGTCCCCCAGATTTGCCTTTCTCT |
| | TGTAAGTTCACTGGCCACCTCTGCTATCACCCTGTTTGGCTC |
| | AAGGAAGGCTGTTTCTCCTCTAGCACTTCTTCCCACG[C/T]C |
| | TTGATAGAGGCTTGTCTTCATTCTCCACAATTTGGCTGATTC |
| | CAGGCCACTGCATTTATTCAGCCTTTTCAGAAGACCCAAAGG |
| | CCAGAAAGTTAATAATATTTCTTTCAATGGAGCCTCTAGATA |
| | TGGTTGTCTGAGAGCTCAGGCCCTTTGCCATGCATCTTCCAC |
| | CCTGTACCTCTTCTGCTGCCAAGTGACTACATCTTTGAATCC |
| | AAGTATATCCACAGCAGCCTTTTCAGAAGACCCAAAGGCCAG |
| | AAAGTTAATAATATTTCTTTCAATGGAGCCTCTAGATATGGT |
| | TGTCTGAGAGCTCAGGCCCTTTGCCATGCATCTTCCACCCTG |
| | TACCTCTTCTGCTGCCAAGTGACTACATCTTTGAATCCAAGT |
| | ATATCCACAG |
| 49 | ACTGTGACTTTTCAAGGCAGAGCCAAGAAAGAGAGGGAGCGA |
| | GAGGGGAATGGCCTATTTCAGAAAGTTTGGGAAGCCATGCAT |
| | GCCAGGTTGGAGCAGAGAACCAGAGGGAAGGGTAAAAAAGGT |
| | GAAGCTGGAAAAGAAAGAAACGATGACACAGAGAGCCTTCTG |
| | GTCCACGTAGAAGATCTGGGACTTCATTGTGAGGCAAGAGTG |
| | GATTTGGA[C/T]GATGAAGTGATGGTGACTCAGGTGACCAGA |
| | GTGGAGGCGGATAAGCAGGAAAAACAGATCAATGTAAGTGGC |
| | ATCCAGGAGGCAGAGGTGACCAGCAGCAGGGAATTTCTGCAA |
| | TATTTTCTCTCTGCTGTCCACATTTGCAAGGTAGAGCCGTTC |
| | ATCTG |
| 50 | CCGTGAGCCACAGGGAGCCACAGGGCTGGGACTAGAGTGATG |
| | ACACGCCTGGAGGATGACTAAATGGCCTTGAAGAATGATTGA |
| | AATTTCGTGCCCTAGGCACCTCCCTCTAATCCCAACCCTGCT |
| | GAGCCACCTCGCCTCCATTTAACCACTCAAATATCTAGTAGG |
| | CATTGACCAACGGCAAAGTATGTTGCATACTT[C/T]GGAGGG |
| | AGTACAAAAAAAGAGACAGGAAAAAGGCACCTGTCTGCTTCT |
| | TCAAAGCAAATAAAAACAGAGACTTAGGAGGCAAAACTGGTG |
| | CTTCTGTTCACAACCCGGCTGGGTGGCTGGGGGAGAATGCTG |
| | ATGCTCTGTTCCTTCTTTGTGCCCAGACTTTTCCACACTGAC |
| | AGAAG |
| 51 | AGGTGACAGGGAGGGAAAGATGATGGCGAGGGGAAAGAGCAT |
| | CGTATAGGCTAGGGGATTGAACTGTGGACTGATTCAGTGTAA |
| | ATAAAAACAAATTAACAGGTAGTAGTTCCTGTCAGTTCTGTT |
| | GGAAGCAGCCCACTCCCTCACTT[G/T]TACTCCTTCCTCCAA |
| | CCCTCAATCAGAGCTACTTACAGCCAGGCAGGATGGGGCTTT |
| | CCCCAAAGCAATTGGCCGTTTGTCCAGTGGCAGACAATGGTG |
| | GCTACAGCCTTTGTGCTTTTAAGAGTGGGCTGGGATTGTGGA |
| | GACTGCCCAGGGGCTTGGCTTTGTCAGAACTTGTACAATCTT |
| | CTTTTGATGAAATTATCTGAACTCAACATCTAGCACATAGAA |
| | TGTGTT |
| 52 | TTAAACTAATTTCATCCCAGCCTAGTCTCCTGATGTCTGTGC |
| | TCGTTATGCTATGCCATGTTGCTTCAGTCAAACAAGAAACAC |
| | TTACTGAGGGCATATTGTTTGCCCATCATTGTGCAATATGCT |
| | GTGGGACATGGGATGTCGTCTCAGTCTTCAAGGAAATGGAAG |
| | GGTTAAGAAATGCATATGAGATTATTACCAATGACACC[G/T] |
| | TTAATTTATATGATGCTTTAAAATTATCAGAGCACTTTCATG |
| | TATTTTTCGTGTGACTAGTGAAAAAGACAATGTTGTACAAAA |
| | TTAAAATATATACAGATGCAATAATGTGAATAGTTAGGATTT |
| | ATTAAATGCATTATCTTCAGATCTTTACATGTATAATCTCAT |
| | TTCTTTCATGCAGTTACTC |
| 53 | GTTTCTGACATTCATGTAAGTGGAAAAATACAGTATTGTAAC |
| | TTTTTTATCTTAATGCAATACAATTTCATTCTTATTAAACAT |
| | AATCATATTGCTTTCCATATTTCCTCATAGCTTCTTGTGAAC |
| | ATTTTGATCTTGGTTCTGCCAACCATATCTGCTAATTTCCCT |
| | AACTTTATGCCACCTACAGACTTAATAATTCCTTCTGTCCTT |
| | TATCCAAC[A/G]TAAGACAAAACCAATGATAGAGTCCAGGGT |
| | ACTTAACTAGAAACCTCCCTTCAATTTTCAAGCTTAGGAAAT |
| | GAGAAGCATTGTGAAAGCGCCAGCCAAATGGGGGGACCTGGA |
| | GACAACACATGTGGATGGAGAGGGCTGTGTTCTGTTAGGATG |
| | CAGTGAAGAGGGAGG |
| 54 | CGTCTCACAAGCCAATGGGAGGAGCTGGGAACCCAGCATTAG |
| | CTGGAAGGTGCTCACCTTCCCTCTTGATGGTTGACTCAGTGC |
| | CGACACAGACTTTAGCTGTCAGCCTGAAAGCAATCAGCTCAT |
| | CATTGCAGCCTCTTTATTGTCTAAATTTTTCTGAA[A/G]GCA |
| | TGAGAAGCCACTGGCCTTTCAACAGGGCTACCGAGGACCTGG |
| | GGAAGAGAAGGGCAGGGAAGCAGAGACAGAGGCCAAACACAA |
| | GTGCTTCTGCCAGCAGGGTTCAGAGCACATCTGGCAACCCCA |
| | GCTCCCAGCCCTGAAGCAGGCACCAGGCTACAAGGACAGACA |
| | TAGACCAGGGACAAGGAAAAGGAAGGGCCAGCCCCAAAGCCG |
| | TTGGGA |
| 55 | TACATGTAAGCTCCACATACCGGGCTATGGACAAAGTATGTC |
| | TTGTCCAACTTTGTTGTCTCAACATGTACCACTTAGCAGATG |
| | CTCAATAAATATTTGTAGGAAGAAGAAGGGCAGGAAGGGGGA |
| | AGGGCAAGAAGAAAAGGCAGTCAA[A/G]GTGAAGCAGCACCC |
| | CTGCCCTTCTGAGCCCCTTTCAGTATCATGCTGACTCTCATC |
| | TTCATGTGGAAAACCTGCCATTTGACTGGGCCAGTTGCACTG |
| | ATTATGTTTTACCGGAGAATGAGCCGAGTGGACTAGGAAAAC |
| | ACTTTAAAAGTCAGTTGTGGGGCAGGGTAGGGGGTGGGGGCA |
| | TTGGCATGAAAATGTTTAGATTTCTTCAGTATTGACTTTGTT |
| | TTGTCAAACAAGT |
| 56 | ACCACTTAGCAGATGCTCAATAAATATTTGTAGGAAGAAGAA |
| | GGGCAGGAAGGGGGAAGGGCAAGAAGAAAAGGCAGTCAAGGT |
| | GAAGCAGCACCCCTGCCCTTCTGAGCCCCTTTCAGTATCATG |
| | CTGACTCTCATCTTC[A/G]TGTGGAAAACCTGCCATTTGACT |
| | GGGCCAGTTGCACTGATTATGTTTTACCGGAGAATGAGCCGA |
| | GTGGACTAGGAAAACACTTTAAAACTCAGTTGTGGGGCAGGG |
| | TAGGGGGTGGGGGCATTGGCATGAAAATGTTTAGATTTCTTC |
| | AGTATTGACTTTGTTTTGTCAAACAAGTCCTCTTGAGGGTAG |
| | GGACCATGTCTAGTTCAGTCTCCGCTGTAGCTCCAGCACCTT |
| | GCATAGTGCGTAGTG |
| 57 | TTACAGAGCAACCAAACAAACACATGTGTGAACTTTCACTTC |
| | ACCTAGACTTAAAAGCCCTAAGGGGGAGGAAAAATCGCAAAG |
| | AAAGCTTTTCTGTGAACTAAGTAAAACCCATCTCCAATTCCT |
| | TTCCTTAATCCTCTACAAGCCTTAGACTATAAGATATCTGGG |
| | TTTC[G/T]GTGGGGAGTGCAGGATGAGTAGGGAGGGAAATCA |
| | GAAAAGAAAGTGAGCAAGTTCATCACCTCCCTTCTTTGCTAC |
| | AACTGTGTATCAAAGGCATGTGGCTCAATGTGACAAAAAGGC |
| | AAAAGCCTGACCTCAAGTGCCCAGGGAGGAGACACAAGCTAC |
| | AATAAGGCTTCCAAGCTCAAGAGACTGAGAGAAAAGTGAGGA |
| | GAAGCTGGAATGAAG |
| 58 | CCTCAGTAATCAAGGTAAATCATATTGCAATGCAGTATTTTT |
| | TAAAAATCAGAGTTATTGAAAAATTCCACGATGAATACAGTA |
| | TCAAAATTTTAAGTCAGGCTTTGTCTGGGTTGGTGTCCAGGT |
| | GTGAAGGATCTCAGGAACAGGCAGCA[C/T]ACTATTTCTGAA |
| | ACCTGGCCTTGGACTTCAGTGAGCGCAGGGCAAGGAACACCA |
| | CGGCCCTGGGGCCCTCGCTCCCCAGGGCTCCGCTTGTGACTA |
| | CTCTGACCTCTGGGTTCAGAATGCCCAGACGTCTATCCCTCA |
| | TGTTCCGGTAGGAGAGGCCTGTCTGTGCACCCCTGGAATGGT |
| | ATTCTGCTGTCCAATCCTGAGACACCTAACTAGTTAGACACC |
| | TATCTCGATAG |
| 59 | GTCCTGACCCCAAGTAGCTAGATGGGTACCCTCAATACTTGC |
| | TGGTTTGCCCTTTGCCGAGAGTTTCCAAAAAGAACAAGGACA |
| | TACTTCATGAACCTCTTCATTGCAACTTTACTGTATTTTTCC |
| | TAACATCCCCGTCCCCC[C/T]GTTCTCACTGAGGCAGTTCCC |
| | CTTTCCACCTGTGGGCTCAGATAAATAGGTGGGGCACCTGGA |
| | AGTGCAGATAGGCAGTGCCAGGTCTATCAAGCCCTATGACGC |
| | CACACTGGGCCACCTTGCTTTCAGTCTTCCTGCTAGCATGAA |
| | AGGGCTTAGGACACAGAAAAGCTCCTATCTCGAACCAAGCCA |
| | GATTCCCATTCCTGGGTGTTGGGGGACAGCGGAGGAGGACCA |
| | AAATCAA |
| 60 | GTTCGAGATAGGAGCTTTTCTGTGTCCTAAGCCCTTTCATGC |
| | TAGCAGGAAGACTGAAAGCAAGGTGGCCCAGTGTGGGGTCAT |
| | AGGGCTTGATAGACCTGGACTGCCTATCTGCACTTCCAGGTG |
| | CCCCACCTATTTATCTGAGCCCACAGGTGGAAAGGGGAACTG |
| | CCTCAGTGAGAAC[A/G]GGGGGAGGGGGATGTTAGGAAAAAT |
| | ACAGTAAAGTTGCAATGAAGAGGTTTCATGAAGTATGTCGTT |
| | GTTCTTTTTGGAAAGTCTCGGCAAAGGGGAAACCAGCAAGTA |
| | TTGAGGGTACCCATCTAGCTAGTTGGGGTCAGGACCTCGTCA |
| | GACCAGGTTCGGATACAATCATCTGCTCATCCCAGGAATAGT |
| | TTC |
| 61 | TAAACATGTGACGGGAACAGAGCTAGGTATCAGAAGTCCAAA |
| | GGTGAGTAATGTAGGGTTTCTGTTCCCAAAGAGGTCACATAT |
| | CTGAGTATAACAAAAAACAGGAGGGCAGGTCAAGCCAAACAG |
| | TTAAATACAACTGTTGTTGACACTTAAATCTCATTGAAAGGG |
| | TATTACTATGC[C/T]CCCTTTTCCTACTGGATTAGAGGCTGT |
| | TTCCAAGTCAGACTTAGGTCTTCCAGGGAGAAAGAGATGGTG |
| | ACAGGAAAGAGATGGTGACAGGAAAGAGTGTGTGTCCCCAGG |
| | TGTCAGATGTCGAGGGCAGTCATATGCAGGGGGATAGCAATT |
| | GCCTGGACTCATCTCCACGCCTGAAGAGGCAGGTATGAGTCA |
| | GGGGTG |
| 62 | GACGCTGCTCTGCCTCTCCTCCATCTCCGAGTGCAACTCCAC |
| | ACGCTCCGTGCTGCGCTCCGACAGCGATGAAATTGTCGTGTA |
| | TGAGATGCCAGTCAGCCCAGTCGAGGCCCCTCCCCTGAGTCC |
| | ATGTACCCACAA[C/T]CCCCTGGTCAATGTGCGAGTAGAGCG |
| | CTTCAAACGAGATCCTAACCAATGTCTGACTCCCACCCATGT |
| | CACCCTCACCACCCCCTCGCAGCCCAGCAGTCACCGGCGGAC |
| | TCCTTCTGATGGGGCCCTTAAGCCAGAGACTCTCCTAGCCAG |
| | CAGGAGCGCCTCCAGCAATGGGTTGAGCCCCAGTCGTGGAGC |
| | AGGTGAGTCTTCTTCCTCTTTTCTCTTTCCTTTCTTTGTGCC |
| | TCCTCAGGGAGTGAA |
| 63 | GAGTCTCTGGCTTAAGGGCCCCATCAGAAGGAGTCCGCCGGT |
| | GACTGCTGGGCTGCGAGGGGGTGGTGAGGGTGACATGGGTGG |
| | GAGTCAGAGATTGGTTAGGATCTCGTTTGAAGCGCTCTACTC |
| | GGACATTGACCAGGGG[A/G]TTGTGGGTACATGGACTCAGGG |
| | GAGGGGCCTCGACTGGGCTGACTGGCATCTCATACACGACAA |
| | TTTCATCGCTGTCGGAGCGCAGCAGGGAGCGTGTGGAGTTGC |
| | ACTCGGAGATGGAGGAGAGGGAGAGCAGCGTCAGGGAGGCAG |
| | AGGGGTCTCCTAGCAACAGCATGGGCTCCTCCTTCTTGAAAA |
| | GCTTTCGGGATGGGGGGCTGGTGGTCCGACGAGGACGGCTGG |
| | ACCTCTGAAAAAGACC |
| 64 | CCCCCAGGACTCAAGCCCACGAGTGAGAACACAAGCACACTC |
| | CAGGGTGTCACCCCTGTTGTGCCCTGCCCAGAGGAGCCATCA |
| | TACATGGCAGTGTGGCTATGGGGTGAGATCTGAAAATAGTTC |
| | TAGCGGCCCTGGAGTTGTATACAACTGTACTACCAG[A/G]GC |
| | TGGGTTATTCTGCCTGCCCCTGCTTCAGCATGAGCCTCTAGG |
| | CAAGAGAGTCCTGTTAACTTCTGCTCTTTCTTCTCTTTCCTC |
| | TGCTTTAAAAATGGAAGAAGAGCCAGGTATAAGCAGAACACC |
| | CTGGCAGAACTTTTTACATTTGGTTACTGCTTGGGTACTTGC |
| | TACTCTGAGTAGAACGCCTTCAGGGGTTGGCAAACTATAACT |
| | GGTGGGTTAAACCCTGCCCGC |
| 65 | GCCCCGGGTGAAGAAACGCAAGGGCAAGTTCAGGAAGAGCCG |
| | GCTGAAGCTCAAGGATGGCAACCGCATCAGCCTCCCTTCTGG |
| | TCAGTGGCCGGGGACTGGCTGGGAAGTGGGAGCAGGGCAAAG |
| | AACTCGTGTGGCAGAGGGTTTGAGGGAG[C/T]CCGGGACTGA |
| | GCTTGGAAACATGACACCCTGGGGCTCAAACTCCAGCCTGTT |
| | TTAAATGGGCCATTCTGTTCACTGGAAAGACTGGCATGTTGA |
| | AAATCTACCCTACTCCAGGGAAATTGTTGCATGTGAGTGAAC |
| | ACAGAATATGAAGTGAGGACAACCCGGCAAGGTCCAGGGTGC |
| | TAGAAGCCTGGTGGCTCACAGGGGCACATCTCACTTTAGGCA |
| | AACCTGGAGAAGG |
| 66 | CGGGAGATTGACATCCTGGAACGGGAGCTCAACATGATCATC |
| | CACCAGCTGTGCCAGGAGAAGCCCCGGGTGAAGAAACGCAAG |
| | GGCAAGTTCAGGAAGAGCCGGCTGAAGCTCAAGGATGGCAAC |
| | CGCATCAGCCTCCCTTGTGGTCAGTGGGCGGGGACTGGCTGG |
| | GAAGTGGGAGCAGGGCAAA[G/T]AACTCGTGTGGCAGAGGGT |
| | TTGAGGGAGTCCGGGACTGAGCTTGGAAACATGACACCGTGG |
| | GGCTCAAACTCCAGCCTGTTTTAAATGGGCCATTCTGTTCAC |
| | TGGAAAGACTGGCATGTTGAAAATCTAGCCTAGTCCAGGGAA |
| | ATTGTTGCATGTGAGTGAACACAGAATATGAAGTGAGGACAA |
| | CCCGGCAAGGTCCA |
| 67 | GAGTAGGGTAGATTTTCAACATGCCAGTCTTTCCAGTGAACA |
| | GAATGGCCCATTTAAAACAGGCTGGAGTTTGAGCCCCAGGGT |
| | GTCATGTTTCCAAGCTCAGTCCCGGACTCCCTCAAACCCTCT |
| | GCCACACGAGTT[A/C]TTTGCCCTGCTCCCACTTCCCAGCCA |
| | GTCCCCGGCGACTGACCAGAAGGGAGGCTGATGCGGTTGCCA |
| | TCCTTGAGCTTCAGCCGGCTCTTCCTGAACTTGCCCTTGCGT |
| | TTCTTCACCCGGGGCTTCTCCTGGCACAGCTGGTGGATGATG |
| | ATGTTGAGCTCCCGTTCCAGGATGTCAATCTCCCGCTCGGCC |
| | AGCTCCTGCTCCCGACGCCGCAGCAGTTCCTCCTGGTTCTTC |
| | TGCTGCAGTGCAG |
| 68 | AAATACTTTTCATAAAAACATGAAAAATGCCACACAATTTCA |
| | GACCCATGTTATTTTTTGCCCATATTCTATTTCTGATTCTGA |
| | TGTCAAAAGACATGGGAAGGATGTTCTGATAACTATCCTCAA |
| | GGTTGAAGTATCCCTATATCAAATTAACCTTCTTCTAGAA |
| | [A/G]TCTATTGTAGAATCAAGAAACATTTCCAGAACTTCTCA |
| | TATTTCCAGCTGGCAGGGTGAGACATTCCACTGTGAGAAAAG |
| | AAAAAAGGCCTGTAATCCAAGCACTGGGATTTTGCCCAACTG |
| | CATTCTTCTCTGGTTTGGACAATTAAGTAGATACCAAGCTGG |
| | AAGGTGGGCATGCTAGAATCAAGATGGTCAGTCAGACTCGGT |
| | CCAGAGGTCCCTGT |
| 69 | GTAAACAAAACAAAAAAAACAAGAAAAAAACGAACGAAAAGA |
| | AAAAAGGCATGCATTGAATTGGTAGACCTCACTCACAGGGAC |
| | CTCTGGACCGAGTCTGACTGACCATCTTGATTCTAGCATGCC |
| | CACCTTCCAGCTTGGTATCTACTTAATTGTCCAAACCAG[A/ |
| | C]GAAGAATGCAGTTGGGCAAAATCCCAGTGCTTGGATTACA |
| | AGGCCTTTTTTCTTTTCTCACAGTGGAATGTCTCACCCTGCC |
| | AGCTGGAAATATGAGAAGTTCTGGAAATGTTTCTTGATTCTA |
| | CAATAGACTTCTAGAAGAAGGTTAATTT |
| 70 | TCAAGGTTGAAGTATCCCTATATCAAATTAACCTTCTTCTAG |
| | AAGTCTATTGTAGAATCAAGAAACATTTCCAGAACTTCTCAT |
| | ATTTCCAGCTGGCAGGGTGAGACATTCCACTGTGAGAAAAGA |
| | AAAAAGGCCTGTAATCCAAGCACTGGGATTTTGCCCAACTGC |
| | ATTCTTC[G/T]CTGGTTTGGACAATTAAGTAGATACCAAGCT |
| | GGAAGGTGGGCATGGTAGAATCAAGATGGTCAGTCAGACTGG |
| | GTCCAGAGGTCCCTGTGAGTGAGGTCTACCAATTGAATGCAT |
| | GCCTTTTTTCTTTTCGTTCGTTTTTTTGTTGTTTTTTTTGTT |
| | TTGTTTACCTTTTCTTTGGCCCTGAGTTGGTCAAACATCTCC |
| | TGAATCTCGT |
| 71 | GTTGTAATGGTGGACAGTTTGCTTTACTTCCTGAGCCTTATT |
| | TGAGGTTACCTAAATTCTGATCTCTGTTTCTGGAAGAAGTAA |
| | GAGGTAGTTTTCTGGGAAGTGAGGCCATGTAATAGCCTAAGT |
| | CTTACATGTTTGCTTCTGTCCATTCATGCTTTCTGAGTATTC |
| | ACATGTT[C/T]TATTCAGACTGCTGGAATCCTGATCCCCACT |
| | CACGACCATCTTTCACGAATATCCTGGACCAGCTAACCACCA |
| | TAGAGGAGTCTGGTTTCTTTGAAATGCCCAAGGACTCCTTCC |
| | ACTGCCTGCAGGACAACTGGAAACACGAGATTCAGGAGATGT |
| | TTGACCAACTCAGGGCCAAAGAAAAGGTAAACAAAACAAAAA |
| | AAACAACAAAAA |
| 72 | CGTTTTTTTGTTGTTTTTTTTGTTTTGTTTACCTTTTCTTTG |
| | GCCCTGAGTTGGTCAAACATCTCCTGAATCTCGTGTTTCCAG |
| | TTGTCCTGCAGGCAGTGGAAGGAGTCCTTGGGCATTTCAAAG |
| | AAACCAGACTCCTCTATGGTGGTTAGCTGGTCCAGGATATTC |
| | GTGAAAGATGGTCGTGAGTGGGGATCAGGATTCCAGCAGTCT |
| | GAATA[A/G]AACATGTGAATACTCAGAAAGCATGAATGGACA |
| | GAAGCAAACATGTAAGACTTAGGCTATTACATGGCCTCACTT |
| | CCCAGAAAACTACCTCTTACTTCTTCCAGAAACAGAGATCAG |
| | AATTTAGGTAACCTCAAATAAGGCTCAGGAAGTAAAGCAAAC |
| | TGTCCACGATTACAAGCA |
| 73 | TTACAGAAGAGTTACAAGGAATCACTAACACCAACAACTAAC |
| | CCTCATCCAGAAATTACCATCTATGGCTAGTAGACCTGTGGT |
| | TTCTCCAAAAGGGCCTGAAATGAGATTGTGTCTGAGGAGGCT |
| | TATGTAGAGCTACAGTGTTGGTGAGA[A/G]CTTTGCTGAGGG |
| | ACACTGGCATTCAGAGAAGAAGTCTTTCTGGGGCTGACTAAA |
| | CTAGTTTTCCTCTGCCAACATCCCGCTTTATGTTTACAATGG |
| | AAGGTGGGTGATATAAATGAAGACTTGGTATTCTGTTGGTGG |
| | GAAAAAGACTGGAGAAATTCAAGAGAAATACAAGTTTACGTG |
| | GATCTGAGCTCAGGGAAAGGCGGGCAGTAGAAGTTGATGTGA |
| | TTTACCTCAG |
| 74 | GGCATTCAGAGAAGAAGTCTTTCTGGGGCTGACTAAACTAGT |
| | TTTCCTCTGCCAACATCCCGCTTTATGTTTACAATGGAAGGT |
| | GGGTGATATAAATGAAGACTTGGTATTCTGTTGGTGGGAAAA |
| | AAGACTGGAGAAATTCAAGAGAAATACAAGTTTAC[A/G]TGG |
| | ATCTGAGCTCAGGGAAAGGCGGGCAGTAGAAGTTGATGTGAT |
| | TTACCTCAGGTTTAGTCAACTTAGTTGCCCTCCCAAGGTAAA |
| | AGGTCAACAAGTCCTTTGATGACCATTTGCAAGCAGCCTGGA |
| | AGAACTCGTTCAGCTAAGTCACTGCCTCTGAGTACCTCCCCG |
| | CAACACAGACCCACCAGCTCCCACAGTGCTGCCAGTAATTTC |
| | CTA |
| 75 | TGGCATAGGGCAAGCATTCCACAGACACTTCTTGAATGAGTC |
| | AGTGAGTTACAAGTCAGTTTGGGAAACAGCTTTGTACCAAGT |
| | TCCTAGGCATGCCCTGGCCCTCCCTGAGACACCCTGCTGGGC |
| | CCTAGGCACGCACGGTGGGCTGATCATTCAGAAGAGGCTGCC |
| | TTAAGTGCAGGAACA[A/G]GACCATACAGATGGTGGCATAAC |
| | TACAAGCACAGATTGTCACTACACACATATCTAGTTCTAATC |
| | ATAGCTTTATGTCTGCCACCTGGGTGAACTTAGGCATGTGAC |
| | TTCAATTGTCTGAGCCCAGTTACCTCATCTGTAAAATGGAAA |
| | TGATGATCCCTACTTTGGGGAATTCTTGTGAAGACTATAGAA |
| | GATGTATAACATTTACCACAGACCCTAGCCCTTAAGCATTTG |
| | TAGGTGGTAACAA |
| 76 | GAAGAGTTCTGTTCTGTGGTTTCCCTGTGATTAATGCAGACT |
| | CTGGTTTCTTCTGTATTC[G/T]TTTCCTTGCCTAGTATTGAT |
| | CCTCCAGAAGGTGGAGAATGGAGACCTGAGCAACAAGATT |
| 77 | CACTGCCTTTGGAAAACATGGAGGCCCGGATGACTTCGGGTG |
| | CCATCCAAGCATACGTCCCTGCCGCACTCATCTTGGTGGTTC |
| | GGTGCCATTCCCGAGCCAGGCCAAAATCAGTGATCCTTCAGA |
| | ATCTTGTTGCTCAGGTCTCCATTCTCCACCTTCTGGAGGATC |
| | AATACTAGGCAAGGAAA[A/C]GAATACAGAAGAAACCAGAGT |
| | CTGCATTAATCACAGGGAAACCACAGAACAGAACTCTTCTGC |
| | CATCCTCCTGGCCTCTCCTGTGGGCTCCAGCATTTAGCTGCC |
| | ATGGATTGATGGTCAGAGCCATCATTTCTTAAACACCTACAG |
| | TGCATCCATTTTTGTACCACCCATTTTATAGCCACTATCTCT |
| | AAT |
| 78 | TATTTTAGTTTCTTCATCAGTAAAACTGAGATGGCAATAGTA |
| | CCTGCCTTGTAAGTTGTTGTGAGGATTAGAGATAGTGGCTAT |
| | AAAATGGGTGGTACAAAAATGGATGCACTGTAGGTGTTTAAG |
| | AAATGATGGCTCTGACCATCAATCCATGGCAGCTAAATGCTG |
| | GAG[C/T]CCACAGGAGAGGCCAGGAGGATGGCAGAAGAGTTC |
| | TGTTCTGTGGTTTCCCTGTGATTAATGCAGACTCTGGTTTCT |
| | TCTGTATTCTTTTCCTTGCCTAGTATTGATCCTCCAGAAGGT |
| | GGAGAATGGAGACCTGAGCAACAAGATTCTGAAGATCACTGA |
| | TTTTGGCCTGGCTCGGGAATGGCACCGAACCACCAAGATGAG |
| 79 | CTTATGCAAAGTTTTCAGACAGGCTGGTTAGTTAACAAGATT |
| | GGGATACTAGGGTGATAGTTTGAACCAGGCCACTCCCAAACA |
| | ACTCTGGTGGCCATAATGTGCATATGTGTTCTGGGGAACAAG |
| | GGAACAAATCAGATGTATTCATGTGGAGCAGGACTGGAGAAA |
| | CAAGGCAAAGCACCAG[C/G]GTTCCTCATAATGTGCCCACAT |
| | CCATGCCTGCCGCATGACTTAACAGCATGAGGTGTGGATTTC |
| | TGCTGCCTTGTTGGGTGGAGGATAGAAGGAGACATGATAGGG |
| | AACTAATATATTTATAGGCCTCCTGTAGGCACAGCACAATTC |
| | ATCAGCCTTGAAAAATGACTCCTTTAACCAGCCCCAACTTTC |
| 80 | TCCCAAACAACTCTGGCTGGCCATAATGTGCATATGTGTTCT |
| | GGGGAACAAGGGAACAAATCAGATGTATTGATGTGGACCAGC |
| | ACTGGAGAAAGAACCCAAAGCACCAGGCTTCCTCATAATGTG |
| | CCCACATCCATGCCTGCCGCATGACTTAACAGCATGAGGTGT |
| | GGA[C/T]TTCTGCTGCCTTGTTGGGCTGGAGGATAGAAGGAG |
| | ACATGATAGGGAACTAATATATTTATAGGCCTCCTGTAGGCA |
| | CAGCACAATTCATCAGCCTTGAAAAATGACTCCTTTAACCAG |
| | CCCCAACTTTCTATGGGAACTTCACTTTCATCTCTTTCTTGG |
| | AGGCCGTACCCACTTCCCACATCTCCCATGCTGCTGAGTGAA |
| | AGGTT |
| 81 | CATAATGTGCATATGTGTTCTGGGGAACAAGGGAACAAATCA |
| | GATGTATTCATGTGGACCAGCACTGGAGAAACAACCCAAAGC |
| | ACCAGGCTTCCTCATAATGTGCCCACATCCATGCCTGCCGCA |
| | TGACTTAACAGCATGAGGTGTGGATTTCTGCTGCCTTGTTGG |
| | GCTGGAGGA[C/T]AGAAGGAGACATGATAGGGAACTAATATA |
| | TTTATAGGCCTCCTGTAGGCACAGCACAATTCATCAGCCTTG |
| | AAAAATGACTCCTTTAACCAGCCCCAACTTTCTATGGGAACT |
| | TCACTTTCATCTCTTTCTTGGAGGCCGTACCCACTTCCCACA |
| | TCTCCCATGCTGCTGAGTGAAAGGTTCTCATGATTAAAACAG |
| 82 | AGATCCTGTTTCCACTGTTGTCTGAAATACTTTGGTCCTAGC |
| | TGGAAAGCCTGGCAGAGTAACAGTGTTCCATTGTCGGGCTAA |
| | AAGAAGGAACACTATCATGAGCATCAGGATGACCTTAGGCAA |
| | GTCAGTCAGCTTCTCTAGTTGACAACTGTAAGGTTCCAGTAA |
| | GCTCTAATCATA[C/T]CTATAAGCAAAAAAGAAAGTCAAGAA |
| | TGACCAGAATCCCAAAATCTATCGCTATTTTCCCTACCCACA |
| | GTCCTTTTCTCCTTCCTCTTATTTATTGTTTTTATTTTTAGT |
| | TTTTTGGTTTTTCTTGGTACTCACTGATTAAGGCATCCTCCT |
| | TCCTCTTGACCCTGAAGGTTAACAGAATGCAGAAACAAAAAC |
| | AAATTTTA |
| 83 | GACAAAAAAAAACACACAAACAAAACAAGATTTTTTGGTTGG |
| | CCACATTGGAATTGCCACCAAACACGTGGGCTGCTACATGCG |
| | AAGTCCTATACTAGGGATGATGTGAGGGGAGAGAGAGATGGG |
| | GGGAGCAAGGGAAGACGAAGGGATGAAAAACAGCAACACCTC |
| | TGCTGTC[A/C]AGAATTCTATTGGGATTTCTATGTTTTCATA |
| | GAAAAAAAGTTATCAGAGACAAAAAGATACTTAATCGCATTT |
| | GCCTCTAAGAAGTAAACTTAGGGTTTGGGAGGGAAGGACAGA |
| | GACTTTTCTTTTTTCTCTCTCTTTTTCTGGAGACAGACTTTT |
| | CTTTGTATTTCAATTTTGTCCTATTTTACCCATGTGTATCC |
| 84 | GACAAAATTGAAATACAAAGAAAAGTCTGTCTCCAGAAAAAG |
| | AGAGAGAAAAAAGAAAAGTCTCTGTCCTTCCCTCCCAAAGCC |
| | TAAGTTTACTTCTTAGAGGGAAATGCGATTAAGTATTTTTGT |
| | CTGTGATAACTTTTTTTCTATGAAAACATAGAAATCCCAATA |
| | GAATTCT[G/T]GACAGCAGAGGTGTTGCTGTTTTTCATCCCT |
| | TCGTCTTCCCTTGCTCCCCCCATCTCTGTCTCCCCTCACATC |
| | ATCCCTAGTATAGGACTTCGCATGTAGCAGCCCAGGTGTTTG |
| | GTGGCAATTCCAATGTGGCCAACCAAAAAATCTTGTTTTGTT |
| | TGTGTGTTTTTTTTTGTCCCTAGTTCCTTTCTGGATGGACTC |
| | AAAACACTT |
| 85 | GGAAAACCAGTCAGGAGCTTTGGCTAGTGTGTGGTCCAGGTA |
| | TACTCCAGGGCTTAGAATGTTAGAGCCTGCTGAGCTGAGGGC |
| | AATGGGAAGACAAAATCTCTATCATCCTCACACTGGTGAAAG |
| | CATGTCTTCAGCTTCCTGACCTTGTGAACTCCAAGAGAAGGT |
| | TGGAGA[C/T]GAGGACAAGGTGAAGTGAAGAGAATTTGGAGA |
| | GGGAGATGCTACATGGACCTCCAGTTCCAACAGGAGCAGCCA |
| | ATCCAACAATTCAACAAAACTGGAACAGGCACCATGAGGAGC |
| | GTGAGAGGCAGTCCCCAAGTTGGGAACAAAGGGAGTAGTGAG |
| | GTTGGACAGAACAGACTGGGAGTAGACCCGACAGCACCGGGC |
| 86 | CCAACAGGAGCAGCCAATCCAACAATTCAACAAAACTGGAAC |
| | AGGCACCATGAGGAGCGTGACAGGCAGTCCCCAAGTTGGGAA |
| | CAAAGGGAGTACTGAGGTTGGAGAGAACAGACTGGGAGTAGA |
| | CGCGACAGCACCGGGCAGTGGTC[G/T]GCTCCAGCAGCTCCT |
| | AGCAGCCGGCAACTGGGTACAGTGGAGACGTGGGCCAGTGAG |
| | TTAGCCAGGGCTGGTGATCAGGAAAAGTGTGAAGAGGGGAGG |
| | AGGCAGAGGCGTTTGGGATTAATAACGAATGTACTGTCACAA |
| | CACCACACTCCAGCATCCAAAACAGGTGGTTGGGCTCAGACA |
| | ATTAAGATGGATCAAGGGCCAGGGGACTCTAACGAGGAT |
| 87 | AAAGAAGCCCGGAGGATTTTAGGGCAGCTCCATATTGTCATC |
| | TGGGACCCAGGCTCCTATTTTTCACTCCCTCATCCCAGTACT |
| | TGGCTTCCATCCTCATTGTCACCCTGTGGTCGAGATGGCTGC |
| | TGGACTGCCAGTTATGAGGTCGAAGTGACAGGCCTGAAGGAT |
| | GAGGGGAA[A/G]CAGAAGCACAGAAAAGGTGCCCCATGTGGC |
| | TGAGTTGGCTCCCATTAATCTCCCGGAAGTCCAGCATGGCGT |
| | ATGTGCTTGTGTTAATTGGACAGAGTGTGGTCGCCTGGCCAT |
| | CACTCGCTGGAGAGGAGACAGGGAGATGGAGGCTTTTATGCT |
| | GGGTGTCATTGAACCCAGCTGAGAATCAGGATCTGTTATCAA |
| | G |
| 88 | CATCCTGGTGAATTGGGCTGTGCAGATTGCCAGAGGGATGAA |
| | CTACTTACATGATGAGGCAATTGTTCCCATCATCCACCGCGA |
| | CCTTAAGTGCAGCAACAGTGAGTATGAAGAGATGGGGCTGGA |
| | GGGGCTCAGAGCA[A/G]TTGCAGTTGTATCCCACGACGTCAG |
| | TAGGAGTGGACTTCTAATGGAACCTTAGAGGGCAGTGAAACT |
| | CCTTGCTTAAACACATTCCATCCTTGAATGGAGATGTAGGAG |
| | GTGAGATGAGTGCCAAGAAAGATTGGTTTAGGAAGAGAGGAA |
| | AGAGGCGCCTGAGGAGTTTGGGATAATTTTGAGCTTTTTTTT |
| | TTAGCAGTCTAGAGTGGCCTTCCCTACTATTTTTCCACATAT |
| | CAGAAT |
| 89 | TGATTCTGTATGTTCCCAATTAGAGAAGTAGCAGCTGACCTG |
| | GTGGGCCAGTTCTGCTATGTTCTGGGAGTTGTTCACATAGGT |
| | CCAGGTGAGAGCCTCTTCTCTCAGCCCTTCGAACAATTCTGC |
| | AAGCACAGAATTTCCTGTGGTAAATGTCTTTCTTGCTCAAAA |
| | TAGCTTGAGAGTTTCTGAGCCCCAGAATGAATCCAGACT[G/ |
| | T]ATTACTAACTTGTGCCTGATGTCATCTATATGAATGAGAA |
| | AAATATGCCTATGTTATAAAGAAAGATTAGAAACTCTCTTGG |
| | AGAAATACATGTTAAGAGTCTTCCTGGTCATGGAGGGAGGAA |
| | CATTCATGTTTTTCTAGCAGCTTTTACACATATGAAAAGATG |
| | TGTGATTACTTAA |
| 90 | CTAAAAATTAAAAAAACAAAAAAAGCTTTTAAATAAAGTTTG |
| | AAGCTAAATTTTCACTTTAAGAAGCAATAATATGAAACTAAA |
| | GCTGAACAGTATTGCTTGACTTAATGTCTTCTATTCATGGTC |
| | CAGAATGCAACGTCACAACACAT[A/G]CTCAGAGGAGAAGTT |
| | CAAAAGGGTAAGTCTGGCGATGAAGTGTCAGTGGCTCAATTT |
| | CATTCCAAGTCTTCGGAGACAAGGTTTGTCAATCGTCTTCAC |
| | TTTCCGAGTGACAGAAAGTGATCTGGTAGAGGACATAGACTT |
| | CAATGACCGCCCATGTTCACCCTCTGATGAACCAAGGGACTG |
| | CACTGAAGTTTTATATTTACATACTTAATCTCCCTCATGTAA |
| | CTTATAAGCAACTTC |
| 91 | TTAAATGACAACAGTCTAGTCCCTGAGAACCTTAACTGTATA |
| | AAATGACTAAGGGCGCAATAATGATTAGAAAACATTCAGACT |
| | CAAATAATAAAATACTTTCTCCTTTAACGTGGTTTTCCAGAT |
| | TTCTACTCAAGGAGACCTTTTAAGTGGCTAT[G/T]CCTTTCC |
| | TAGTTGAAGGCTGTGAATCTTTTGCTCACAGACATGAGACAT |
| | GAGTCTCCTGACACTGATGAGACCTTTAAGACAGTTGCCTGT |
| | TCTCTGGGTTACTGGATGGAGCCCTTTGTGAAGTCAGTGGGA |
| | GCCCCAGAGAGAAGCCTGCTTGGGCGGTGGAACATCAGGGAA |
| | ACTCTCTTGTGCTTTGTGTTTTATGAAAAGTAGGGGAGATTC |
| | CAACCTGAAGCCT |
| 92 | ACTCCGTCTCAAAAAAAAAAAAGAAAAGAAAAAAAAAACCCA |
| | AACTGCATCCAGAGAAAGCATGTAGACGTTCAGAGAGGAATG |
| | AAAACAAGAGGCTCCCTGTCATTCAGGCCTTGGTTCCCATGT |
| | GTCCCTGCCTGCTGTAAACAATATCCCACCAAGGGCCAG[C/ |
| | G]TTTTGCTAAAGATAGCTCTGGTGGGCTTCAATCACCATTA |
| | AACAGTTCCTAAACTAATTCTGCTAAGGCTAATGTAGAGCAG |
| | AAGACTCCCTTGGCCCATGTAACAGCTGTGTCATCATTCATT |
| | TACTCATCCCATCAGTCAATGAATATCATTGAATGCCAGCTG |
| | TTTACAAGCTTGTAAGAAGAGGAAGAAAAAACATTGGCTGCA |
| | GTCTTTA |
| 93 | CAGGTATTGTTTCGAAAGCTCATTCAAAAAGTGCTAGGTAAC |
| | TAGGCCAGGAAAGGTTTCCAGCCTAACATACTCTGCCCATTC |
| | CAGCCTTTCCAATATGATAACCTAAGTGCTGGTGTTCAGACA |
| | AATTGCCATTTCGGTCAACTCTTGTCATCAACTTAAGTTGCC |
| | CCTTGGG[G/T]AGGCTTAATGTCCCTTTCTGCCTTCTGGACT |
| | TGGTTGGGATCATCTTAGGCATTGGAAGTATCAAATAATGTT |
| | AAACCTCGAGAGATGTAGCTTGGGTATTAGGTCCCTTTCATT |
| | TAAGTAGGGTCCTTGGCCCCCAAAACACGTTTCTCCAACCTC |
| | TTTGGAAGTCAGCTACATACCAGGGATTAGACCAAACCTTA |
| 94 | CTTTCTACCCAAGAATCAGCAGTTTAATAGTTTCCTCATTCA |
| | TCATACCTGTAGAGCACTGCACTAGGAGAAAAAGAAATAAGA |
| | TAGAGGATTTCCCAAATGAAGAGGATAAAGGCCAAGGCAAGG |
| | CTGCAGCAGCAATCTGAGCTGTCCTGTGCGAAAGATGACAGG |
| | ACCACTGGGAGAGAAA[A/C]AGGGGAACAGGGACGGACAAGA |
| | GAGAAGAGGGAGTAATCAGTAGTCTGAAGACTGCTGGAGAAC |
| | AACTTGACATTGTGTATTTAAGGACAGCCTCAACAAAGAAAC |
| | CTTATTAGCCAGAGCTATCAAAATAGACCCACTTACTAAATA |
| | TGTTTCCAAACTCTTCACTCCCACTCAA |
| 95 | TGGAACCAATCGCCCATGACTACTGAGGGATGACTGTACTAA |
| | CCACTCCTCCAAGTAGCATGGAGCAGCTACTTGGGAGCAGTG |
| | AGCAGAGAAGCGTGTAGGAAGTGTGTGTGAAGTGTGAGCTCT |
| | GCTAGCATCGAAAGAAAGAGAGGAAGCAAAA[C/T]GACTGAC |
| | TTCCCGGCCCTGCCTCCCCTGCCATGTCAGATGGAAGCTTGG |
| | AGACTGACCCTTGAAAGCTCCAACTAAGGACGAAAAGAGAGT |
| | GAGGTGTGGAGAGCTTAGTGGGCCCTAACATGCCCACCAGAC |
| | ACAAGAGGCAATA |
| 96 | CTAACTTTCTTAAAAAAAAAAAAAAAAAGAAAGAAGAATATA |
| | TGTGAACCTAAAAGTAAACAGGAACATCTACGAAGAGGAGCG |
| | GGAATGAGTGTTGGTAGGTGGGTAGGAGACCACAGTTGCGCA |
| | CCTAGCC[G/T]GATCTCTGCCTAACTGCAGGTGTTCTACAGG |
| | CTCCTTCCATAGAGATGACAGGATGCATGGAAGGAAGAGCAC |
| | CCTGGAAGGAAAACACACAGTTCCAGAGGCCTCTTCAAACAG |
| | TACGAGTAATGCATACATAGAAGTGCAAAGGTGTGTACAGAT |
| | TATTTGTTCCCTATAGATACAGAGCCAGCAGTACTTAAGCAC |
| | ATAAATCCAGCTTTTCATCAATGAGAAGC |
| 97 | AGTTGCCCACCTAGCCTGATCTCTGCCTAACTGCAGGTGTTC |
| | TACAGGCTCCTTCCATAGAGATGACAGGATGCATGGAAGGAA |
| | GAGCACCCTGGAAGGAAAACACACAGTTGCAGAGGCCTCTTC |
| | AAACAGTACCAGTAATGCATACATAG[A/G]AGTGCAAAGGTG |
| | TGTACAGATTTATTTGTTCCCTATAGATACAGAGCCAGCAGT |
| | ACTTAAGCACATAAATCCAGCTTTTCATCAATGAGAAGCCAA |
| | ATGAGTCTCTCATTCATTTATTACTGGATCCACCTGTCTATA |
| | TAAAGCCCATCTTCATTTTGCCTGCTTGGTAAAAAAGGAAAC |
| | TCTCATTGGCTCACCTAGCTCTTAGAGTATAAG |
| 98 | GCAAAGGCAGAAAATAACTGACCTTTGGTCAGGACACAGTCC |
| | ATTTTCTGGGTTCTGGCCTAGACATGCAGCTCATGAAAGATT |
| | CAGACTTTATTTCTTATAGTCTAAGAGCTAGGTGAGCCAATG |
| | AGA[G/T]TTTCCTTTTTTACCAAGCAGGCAAAATGAAGATGG |
| | GCTTTATATAGACAGGTGGATCCAGTAATAAATGAATGAGAG |
| | ACTCATTTGGCTTCTCATTGATGAAAAGCTGGATTTATGTGC |
| | TTAAGTACTGCTGGCTCTGTATCTATAGGGAACAAATAAATC |
| | TGTACACACCTTTGCACTTCTATGTATGCATTACTGGTACTG |
| | TTTGAAGAGGCCTCTGGAACTGTGTGTTTTCCTTCCAGGG |
| 99 | TAAATGTTGAGATAGACTGGCTTCCTGGCTTACCCAAAGTGA |
| | GCCAGGGTAAGCTCACTTACCTTTAGCCAGAGGTGAGCTAAA |
| | ACCTGAATTGAGCACATTTCAGTTGCATTGTATAGGCAATGT |
| | TTTTGCTCATTTTTTTTCCATTGCATAACTTTATTCCACTC |
| | [C/T]CTGAGATGTTTCTCCATGCCCTTTTCCATCATTTTTTC |
| | ATATCTCCCTTGCTTCCATTTTTACTCTCTCCTTCTTTCCTC |
| | CAATAAAAAAGGGCTTCTTGGGTCACACCTCCATCAACAGAC |
| | GCTTGGAAATAACGCACCATGGCAGGTTTTCAAATATTTTTT |
| | CTTTCCTCCCTCCAGAGAGAAAAACAAAAGGGTAAAGATGGC |
| 100 | GGAAGAGGAGAAAATGGAAAGTTGTTTAATGGGTACAGAGTT |
| | TCAGTTCTGCAAGATGGAAAAGTTCTGGAAATCTATTGCAAA |
| | CAATGTGCAGACGCTTAACACTAATGAACTGTACCCTTAAAA |
| | ATTATAGACATGGTACATTTTATGTGCGCTTTTTTAACCACA |
| | GTTGAAAATTTTTTTTGAAGA[A/G]GCTGAAGTTTGCTGATT |
| | TTCTTTGAGACTTACTGCATCACTCAGTATCAGAAAATCTAA |
| | AAACAGGGGGACTGTGCAACTGATGAAACCAAATCCCTCATT |
| | ATGGTGTGTTTCTGTGTTAGACACCATAATCCCAAATTGCAC |
| | AGGTCTGCCTTTCCTTAAAGGATGTAACTGACTGGACTAAG |
| 101 | AGGAACCTGACCGTGCTGGGACCTTGATACTCATACTTCCAG |
| | CCTCCAGACATGTCAGAAAATAAATTTTTGTTGTTTAAGCCA |
| | CCCAATCCATAACATAGACTGGGTATLTATGTCACCCAGTGC |
| | CATAACATGTTATGCTATGGCAGCCTAAGCTGAGTTAGATAG |
| | TAACCATGATTATGTGACA[A/T]AAAAGGAGCATAGTAAAGA |
| | GAGCACATAATTCTAGAAGGGGTGGAGGTTGGAGAGAAATTA |
| | AGTTGAGAAGAGGAGAACAAGATGTCAGGAAAGTCTTCCCAT |
| | ATGAGATACTTCTAGGAATTGAGTCTAGTCTAGAGGAACAGG |
| | GAGTTATTCTCTAAATGGTCTGTAAGAAGAGCTGGGGGCCTT |
| | AAAAA |
| 102 | TCTGATTTCTAGCCTATTTTCCTTCCAATGGCACTGTCCTAA |
| | GGTTGTACAGCTTAGAAAATGTTAACCCAGTATTGTTTTCTT |
| | TACTTGCATGTGAGAGAAGAGAAGTCAAAGTGAAAAGCAGGC |
| | AGCTGGAAACCTCCAGGGGAAGGGATTCCCAGGG[G/T]TCCT |
| | TTATATTCCTTTCTCCCCAAGCAAAGCAGAGAGAGAAGCAGC |
| | ACACATACCCTGTGGATTCTGGGTTTGGAAAGCATAGTTGTG |
| | TTGTTCATCTGGGCTGCTATAACAAAATACTATACACTGAAT |
| | GGCTCATAAACCACAGAAATGTATTTCTCACAGTTCTGGTGG |
| | CTTGGAAGTCCAAAATCAAGGTGCCAGCAGATTTTGTGTCTG |
| | GTGAG |
| 103 | GAAAAGAGATCAGTGGAGAATGTGGCTGATCGTGCAGCCTTG |
| | GTGATAGATCTGACTATGGTAGTGCACATACTACATTTAAGC |
| | CCTGCTGTGTATCAAAGCTGGGTAGTCAACCCTAACACATAC |
| | CCTTCACTTATAATGAAACT[C/T]CTAATGGACTCTTGCTTA |
| | TAGGGAAAGCTTGTATAACCAACTCTCAATTTTTATGTGTAT |
| | GTAAGGTCAAAACTGCAGTGTTGTGTTAATGGTGATACCACT |
| | ACAATGTCTGTAGATATAGGCAATAGAAAACTGAAGCACTGT |
| | TATGACACAAATACAGTGTAAACATTCTTGAAAAACTGGGAA |
| | TTTGCTGCAGCATCACTCCTAGCCACTCCCCAGAAATTTGGA |
| | GG |
| 104 | TGTCTCTGAAGTTTGTCAGACTTAGCAAACTGCCAAAGCACA |
| | TGGCATGTGTTGATATAAAAACTTTCCAAATCAGGCTCTGAA |
| | CTCCAATTAGAATATAGTACAGAAGCAGAGGGAGGAAGGGCT |
| | GGAGAGACCAGGACAAAAGTTATCACACATTTACACTGGTTG |
| | TGATTGGG[C/T]AGTGGGCCTATTAACAAAGCTTTCTCTCTC |
| | TTTCCTATATTTTCTCAGATAAAAATACCATGAATGTGTTAC |
| | TTTTAGGATGAAAAACTAAATACAAAAGACCAATTGGATGTA |
| | AAATGGTGGAGAGGCTGGCCCAAGCAAACTCTCAATAAGTTG |
| | TCAGGTTTTCAAAATACAAGTCCAAAGGAAGAGTGGATAGAT |
| | A |
| 105 | CACTCATTTATTAATTTCTGCCTCTGTGCTTTTACTGACACC |
| | ATTTGCCTTTACTTAGAAAACATTACGCCTCCACCTGCCTGC |
| | TAAAATCCACTACGTCTACTCCAAACCCATTCTTCTATTTTT |
| | TGAACAGCCTAGAGCACTCACATGTTCCACTCATTTAGCGTT |
| | TAATTATGTGCTACTTCA[A/G]ATCCCTCCATCTAAACTGTA |
| | CATTCCTTGAGGGCAAGGCACTTCTCAGCATGTTCCACAGGG |
| | CCTAGCAGAGAAAGCTGTTAGCATCAATTAACACTCAGTTCA |
| | TATACATTGATTAAACAAAGCACAGAAACCAATGGCTTTCCA |
| | ATTAGAGATTTTAATCTGTTTTTTCTTTTTTTCTT |
| 106 | CAGGTTCTGGGATGAATCCCAGTCATTTTGGAGGGCAACTAG |
| | AGGTTTATATTAACACTCTGGGAGTTCAACATAAGGCAACCA |
| | TGCTCAGTGTGAAGTTTGGTCCTAGTTCTAGGTATTAGGCTC |
| | CTGCTTTGTTTGTAAGTCTCA[C/T]TCTGTCCCCCCAGATCC |
| | AGCCTTTGAATATTTGTTTTGTCCTGCTGGGTCCTCAAACTT |
| | GGGCCTCCAGTGAGTATCTCAGTAGCTCAAGGATCTGAGTTC |
| | TATTTGTTTTCATCAATTCGCTGTAGAAACGAAAGGGCAGAC |
| | CTAGAAAACCCCAGCACTGAAGCTGGGGCCTTGCTATAAGCA |
| | ACACACTTTCTCAAGGCTTGCCGT |
| 107 | CAAGAATGATTCCGTTTTTACAGCTATAAAAAGAGATCTACA |
| | ATGTCAATTTGTAGAAAAGTGTATTAATTTACAAAAAAAGCA |
| | GTTGATTTATAAAATGTTCTGTCTTTTGTGACGTAAAGGTGG |
| | AGTGTTGCACATTTAAAAA[C/T]ACAATCACCGCGTACAAAT |
| | GGTAAGCTGTCCACTCCATACTTGCATGTCCTGTGCCGCTTC |
| | TATTAACTTGTCTTTGGAAGTAGTAAATTCTCCCAGTCTGGA |
| | CGTGGCTATTTTTTTTTCTCAGACACGGGCCAACAGAGTTCT |
| | TGCTCAACTTGGGTGCGTTTATGACTTTTCAACAGTGTCATC |
| | AGCCGTACCTAACTCTTCCAGGCCTTCAGTATCTCA |
| 108 | TAAAGGAAAAGAAAATTAGACCTCCAAGAAAGCAGCCATATT |
| | TGTCAATTTGTAGAAAAGTGTATTAATTTACAAAAAAAGCAG |
| | TTGATTTATAAAATGTTCTGTCTTTTGTGACGTAAAGGTGGA |
| | GTGTTTTCTCCCACTTGAAAATTACTCCAAATCATAAAATCA |
| | TTTGGT[A/G]ATTGGAAAACTAGGAAAGTTTCTATTTTTTTT |
| | CTAGCCTTTGAATACACAGATTCAGTCAGGTAATTTGCTTTC |
| | TCTAGCTGACTTTACTGAGAGTTAAATCTTTCAACATTTTAT |
| | TAAAGAATTTTCAAACATATAGGAAAGTTGAGAAAATTGTAC |
| | CCACCACCTGGATTCTGTGTTTTTAAATATTGTGTCTCTATC |
| | TGTTTTATT |
| 109 | TGAGAATAAAGATAGGTAATCTTAACAAAAATATGCTTTGTT |
| | ACCAAAAATCTTACTTTACTCCCTTAATGTCACAGTATTGAA |
| | TATTGAAGAATACCAGGATGTGTTATTGAAAAATATACTTAA |
| | AATACTTTAAATATACTTGAAGTGATCTTTTTTCAATTTCTG |
| | [A/T]ATATGAAGATTTTCAAATGTATAAGAAAATTGAAAGAA |
| | TGCTTCATCTAGATTCCACTATTATGAACCTTTTTGAAAAAT |
| | CACATTTGTTTCAACTATATAAAGATGCATGGTATTTATCTG |
| | TACATACTCTGTCTCTCTTACACACATATGTGACTCAATTGA |
| | CAGTATGTTGCAAAGATTATGACACATCACCCCCTAAATGCT |
| | TCAGGCAATATTTTTA |

### REFERENCES

American Thoracic Society, Standardization of Spirometry, 1994. Update. Am. J. Respir. Crit. Care Med., 152: 1107-1136 (1995).
Bleecker E R, et al., Evidence for Multiple Genetic Susceptibility Loci for Asthma. Am J Respir. Crit. Care. Med., 156:S113-6 (1997).
Cockcroft D W, et al., Bronchial Reactivity to Inhaled Histamine: Asthma Method and Clinical Survey. Clin. Exp. Allergy, 7:235-43 (1977).
Collaborative Study on the Genetics of Asthma. A Genome-wide Search for Asthma Susceptibility Loci in Ethnically Diverse Populations. Nature Genet., 15:389-392 (1997).
Cookson W O, The Alliance of Genes and Environment in Asthma and Allergy. Nature, 25:B5-11 (1999).
Cookson W O, and Moffatt M F, Genetics of Asthma and Allergic Disease. Hum. Mol. Genet., 9:2359-64 (2000).
Dempster A P, et al., Maximum Likelihood from Incomplete Data via the EM Algorithm. Statist. Soc., B39:1-38 (1977).
European Community Respiratory Health Survey Group (ECRHSG), Genes for Asthma? An Analysis of the European Community Respiratory Health Survey. Am. J. Respir. Crit. Care Med., 156:1773-1780 (1997).
Fjalldal J B, et al., Automated Genotyping: Combining Neural Networks and Decision Trees to Perform Robust Allele Calling, Proceedings of the International Joint Conference on Neural Networks, A1-A6 (2001).
Gudbjartsson D F, et al., Allegro, a New Computer Program for Multipoint Linkage Analysis, Nat. Genet., 25:12-13 (2000).
Gulcher J R, and Stefansson, K. Population Genomics: Laying the Groundwork for Genetic Disease Modeling and Targeting. Clin. Chem. Lab. Med., 36:523-527 (1998).
Gulcher J R, et al., Protection of Privacy by Third-Party Encryption in Genetic Research in Iceland, Eur. J. Hum. Genet., 8:739-742 (2000).
Gulcher J R, et al., The Role of Linkage Studies for Common Disease. Curr. Opin. Genet. Develop., 11:264-267 (2001).
Helgason A, et al., mtDNA and the Origin of the Icelanders: Deciphering Signals of Recent Population. Am. J. Hum. Genet., 66:999-1016 (2000).
Holloway J W, et al., The Genetic Basis of Atopic Asthma. Clin. Exp. Allergy, 29:1023-1032 (1999).
Kauffmann F, et al., Epidemiologic Study of the Genetics and Environment of Asthma, Bronchial Hyperresponsiveness, and Atopy. Chest, 121(3 Suppl):27S (2002).
Kong A, and Cox N J, Allele-sharing Models: LOD Scores and Accurate Linkage Tests. Am. J. Hum. Genet., 61:1179-1188 (1997).
Koppelman G H, et al., Genetic and Environment in Asthma: the Answer of Twin Studies. Eur. Resp. J., 13: 2-4 (1999).
Kruglyak L, et al., Parametric and Nonparametric Linkage Analysis: a Unified Multipoint Approach. Am. J. Hum. Genet., 58:1347-1363 (1996).
Mansur A H, et al., Suggestive Evidence for Genetic Linkage Between IgE Phenotypes and Chromosome 14q Markers. Am. J. Respir. Crit. Care Med., 159:1796-802 (1999).
National Institutes of Health. 1997. Guidelines for the Diagnosis and Management of Asthma: Expert Panel Report 2, July 1997.U.S. Government Printing Office, Washington, DC. NIH Publication No. 97-4051.
Nicolae D L. Allele Sharing Models in Gene Mapping: A likelihood Approach. Ph.D. Thesis, Department of Statistics. University of Chicago, Chicago 126 (1999).
Palmquist M, et al., Hyperreactivitet hos 420 patienter med nydebuterad astma, Hygiea, 97:108 (1988).
Palsson B, et al., Using Quality Measures to Facilitate Allele Calling in High-throughput Genotyping. Genome Res., 9:1002-1012 (1999).
Weiss S T, Epidemiology and Heterogeneity of Asthma. Ann. Allergy Asthma Immunol., 87 (Suppl 1):5-8 (2001).

The teachings of all publications cited herein are incorporated herein by reference in their entirety. While this invention has been particularly shown and described with references to preferred aspects thereof, it will be understood by those skilled in the art that various changes in form and details may be made therein without departing from the scope of the invention encompassed by the appended claims.

1. Use of a MLK family kinase inhibitor for the manufacture of a medicament for treatment for asthma in an individual in need thereof, wherein the individual has at least one risk factor selected from the group consisting of: an at-risk haplotype for asthma; an at-risk haplotype in the map3k9 gene; a polymorphism in a map3k9 nucleic acid; dysregulation of map3k9 mRNA expression, dysregulation of a map3k9 mRNA isoform; increased MLK1 protein expression; increased MLK1 biochemical activity; and increased MKL1 protein isoform expression:

2. The use of claim 1, wherein the MLK family kinase inhibitor is a MLK1 inhibitor.

3. The use of claim 2, wherein the MLK1 inhibitor is CEP-1347 (Formula III) and its optically pure stereoisomers, mixtures of stereoisomers and salts.

4. The use of claim 2, wherein the MLK1 inhibitor is an indolocarbazole derivative and its optically pure stereoisomers, mixtures of stereoisomers and salts.

5. (canceled)

6. Use of a first nucleic acid molecule for diagnosing asthma or a susceptibility to asthma in a sample from an individual to be diagnosed, comprising detecting the presence or absence of a second nucleic acid molecule of at least one marker of an at-risk haplotype associated with the MAP3K9 gene selected from the group consisting of: haplotype 1, 2, 3, 4, 5, 6, 7, 8, 9 and 10 of Table 1 and combinations thereof in the sample by contact with the first nucleic acid, wherein the presence of one or more markers is indicative of asthma or a susceptibility to asthma.

7.-10. (canceled)

11. A method for the diagnosis and identification of susceptibility to asthma in an individual, comprising: screening in a sample from the individual to be diagnosed for at least one at-risk haplotype associated with MAP3K9 that is more frequently present in an individual susceptible to asthma compared to an individual who is not susceptible to asthma wherein the at-risk haplotype increases the risk significantly.

12. The method of claim 11, wherein the significant increase is at least about 20%.

13. The method of claim 11, wherein the significant increase is identified as an odds ratio of at least about 1.2.

14. (canceled)

15. (canceled)

16. A method of treatment for asthma in an individual, comprising administering a MLK family kinase inhibitor to the individual in need thereof, in a therapeutically effective amount, wherein the individual has at least one risk factor selected from the group consisting of: an at-risk haplotype for asthma; an at-risk haplotype in the MAP3K9 gene; a polymorphism in a MAP3K9 nucleic acid; dysregulation of MAP3K9 mRNA expression, dysregulation of a MAP3K9 mRNA isoform; increased MLK1 protein expression; increased MLK1 biochemical activity; and increased MKL1 protein isoform expression.

17. The method of claim 16, wherein the MLK family kinase inhibitor is selected from the group consisting of: compounds of Formula I, Table A and Table B, and their optically pure stereoisomers, mixtures of stereoisomers and salts.

18. The method of claim 16, wherein the MLK family kinase inhibitor is a MLK1 inhibitor.

19. The method of claim 18, wherein the MLK1 inhibitor is CEP-1347 (Formula III) and its optically pure stereoisomers, mixtures of stereoisomers and salts.

20. The method of claim 18, wherein the MLK1 inhibitor is an indolocarbazole derivative and its optically pure stereoisomers, mixtures of stereoisomers and salts.

21. The method of claim 16, wherein the MLK family kinase inhibitor is an inhibitor of a member of the JNK pathway.

22. The method of claim 21, wherein the MLK family kinase inhibitor is an inhibitor to a member of the MLK kinase pathway.

23. A method of assessing response to treatment with a MLK family kinase nucleic acid inhibitor by an individual in a target population, wherein the individual has at least one risk factor selected from the group consisting of: an at-risk haplotype for asthma; an at-risk halotype in the MAP3K9 gene; a polymorphism in a MAP3K9 nucleic acid; dysregulation of MAP3K9 mRNA expression, dysregulation of a MAP3K9 mRNA isoform; increased MLK1 protein expression; increased MLK1 biochemical activity; and increased MKL1 protein isoform expression, comprising:
a) assessing the level of a MLK1 protein in the individual before treatment with a MLK family kinase nucleic acid inhibitor;
b) assessing the level of the MLK1 protein in the individual during or after treatment with the nucleic acid inhibitor;
c) comparing the level of the MLK1 protein before treatment with the level of the MLK1 during or after treatment,
wherein a level of the MLK1 during or after treatment that is significantly lower than the level of the MLK1 before treatment, is indicative of efficacy of treatment with the nucleic acid inhibitor.

24. The method of claim 23, wherein the nucleic acid inhibitor is RNAi.

25. The method of claim 23, wherein the level of the MLK1 in steps a) and b) is assessed by measurement of ex vivo production of the MLK1 in a sample from the individual.

26. A method of treatment for asthma in an individual with an at-risk haplotype for asthma, comprising administering a MLK family kinase inhibitor to the individual in need thereof, in a therapeutically effective amount.

27. The method of claim 26, wherein the MLK family kinase inhibitor is selected from the group consisting of: compounds of Formula I, Table A and Table B, and their optically pure stereoisomers, mixtures of stereoisomers and salts.

28. The method of claim 26, wherein the MLK family kinase inhibitor is a MLK1 inhibitor.

29. The method of claim 28, wherein the MLK1 inhibitor is CEP-1347 (Formula III) and its optically pure stereoisomers, mixtures of stereoisomers and salts.

30. The method of claim 28, wherein the MLK1 inhibitor is an indolocarbazole derivative and its optically pure stereoisomers, mixtures of stereoisomers and salts.

31. The method of claim 26, wherein the MLK family kinase inhibitor is an inhibitor of a member of the JNK pathway.

32. The method of claim 31, wherein the MLK family kinase inhibitor is an inhibitor to a member of the MLK kinase pathway.

33.-115. (canceled)

116. The method of claim 16, wherein the MLK family kinase inhibitor is selected from the group consisting of: compounds of Formula IV, their optically pure stereoisomers, mixtures of stereoisomers and salts
wherein A represents O or S;
W represents O, NH, NR1;
R4 and R5 are independently selected from the group represented by hydrogen, halogen, cyano, nitro, C1-6-alk(en/yn)yl, C1-6-alk(en/yn)yloxy, C1-6-alk(en/yn)yloxy-C1-6-alk(en/yn)yl, C1-6-alk(en/yn)ylsulfanyl, hydroxy, hydroxy-C1-6-alk(en/yn)yl, halo-C1-6-alk(en/yn)yl, halo-C1-6-alk(en/yn)yloxy, C3-8-cycloalk(en)yl, C3-8-cycloalk(en)yl-C1-6-alk(en/yn)yl, acyl, C1-6-alk(en/yn)yloxycarbonyl, C1-6-alk(en/yn)ylsulfonyl, —NR7R8 and R7R8N—C1-6-alk(en/yn)yl-;
R3 represents hydrogen, halogen, C1-6-alk(en/yn)yl, C3-8-cycloalk(en/yn)yl, aryl, a heterocycle, hydroxy, hydroxy-C1-6-alk(en/yn)yl, C1-6-alk(en/yn)yloxy, C1-6-alk(en/yn)yloxy-C1-6-alk(en/yn)yl, C3-8-cycloalk(en/yn)oxy, C1-6-alk(en/yn)ylsulfanyl, acyl, R7R8N—C1-6-alk(en/yn)yl or —NR7R8;
or R3 represents a group of the formula —R9-Ar2
wherein R9 represents O, NH, NR1′, S, —CONR1′, —CO— or C1-6-alkyl, C2-6-alkenyl, which may optionally be substituted by OH, halogen, C1-6-alkoxy or C3-8-cycloalkyl;
R6 represents C1-6-alk(en/yn)yl, C3-8-cycloalk(en/yn)yl, C3-8-cycloalk(en)yl-C1-6-alk(en/yn)yl or Ar1;
Ar1 and Ar2 are independently selected from the group represented by aryl, a heterocycle or a carbocycle all of which may be substituted one or more times by halogen, cyano, nitro, C1-6-alk(en/yn)yl, C1-6-alk(en/yn)yloxy, C1-6-alk(en/yn)yloxy-C1-6-alk(en/yn)yl, C1-6-alk(en/yn)yloxy-C1-6-alk(en/yn)yloxy-C1-6-alk(en/yn)yl aryloxy-, aryl-C1-6-alk(en/yn) yloxy, halo-C1-6-alk(en/yn)yloxy, C1-6-alk(en/yn)yl-sulfanyl, hydroxy, hydroxy-C1-6-alk(en/yn)yl, halo-C1-6-alk(en/yn)yl, cyano-C1-6-alk(en/yn)yl, NR7R8, NR7R8-C1-6-alk(en/yn)yl, C3-8-cycloalk(en)yl, C3-8-cycloalk(en)yl-C1-6-alk(en/yn)yl, C1-6-alk(en/yn)ylsulfonyl, aryl, acyl, C1-6-alk(en/yn)yloxycarbonyl, C1-6-alk(en/yn)yl-CONR1′-C1-6-alk(en/yn)yl, C1-6-alk(en/yn)yl-CONR1′-, —CONR7R8 or R7R8NCO—C1-6-alk(en/yn)yl;
R7 and R8 are independently selected from the group represented by hydrogen and C1-6-alk(en/yn)yl which may be further substituted by hydroxy, halogen, C1-6-alkoxy, cyano, nitro, C3-8-cycloalk(en)yl, C3-8-cycloalk(en)yl-C1-6-alk(en/yn)yl, aryl or a heterocycle; or R7 and R8 together with the nitrogen to which they are attached form a 3-7-membered ring which optionally contains one or more further heteroatoms and may optionally be substituted by halogen, C1-6-alk(en/yn)yl, hydroxy, hydroxy-C1-6-alk(en/yn)yl or acyl;
the aryls may be further substituted by halogen, cyano, nitro, C1-6-alk(en/yn)yl, C1-6-alk(en/yn)yloxy, C1-6-alk(en/yn)ylsulfanyl, hydroxy, hydroxy-C1-6-alk(en/yn)yl, halo-C1-6-alk(en/yn)yl, halo-C1-6-alk(en/yn)yloxy, C3-8-cycloalk(en)yl, C3-8-cycloalk(en)yl-C1-6-alk(en/yn)yl, acyl, C1-6-alk(en/yn)yloxycarbonyl, C1-6-alk(en/yn)ylsulfonyl, or —NR7′R8′ wherein —NR7—R8′ is as defined for —NR7R8 above provided that any aryl substituent on —NR7′R8′ is not further substituted; and R1 and R140 are independently selected from the group represented by C1-6-alk(en/yn)yl, C3-8-cycloalk(en)yl, aryl, hydroxy-C1-6-alk(en/yn)yl, C3-8-cycloalk(en)yl-C1-6-alk(en/yn)yl and acyl;
or a pharmaceutically acceptable salt thereof.

117.-148. (canceled)
